(19) United States
(12) Patent Application Publication
(10) Pub. No.: US 2024/0415819 A1
(43) Pub. Date: Dec. 19, 2024

(54) MACROCYCLIC COMPOUNDS AS PROTEASOME SUBUNIT BETA TYPE-5 INHIBITORS

(71) Applicants: Lead Discovery Center GmbH, Dortmund (DE); QLI5 THERAPEUTICS AG, Dortmund (DE)

(72) Inventors: Uwe Koch, Dortmund (DE); Gunther Zischinsky, Dortmund (DE); Anke Unger, Dortmund (DE); Peter Nussbaumer, Dortmund (DE); Bert Klebl, Dortmund (DE); Axel Choidas, Herdecke (DE); Klaus Dinkel, Trippstadt (DE); Julian Engel, Dortmund (DE); Carsten Degenhart, Dortmund (DE); Robert Huber, München (DE); Kiyean Nam, Gyeonggi-do (KR); Jaesung Kim, Seoul (KR); Jeongjea Seo, Seoul (KR); Seung-Joo Lee, Gyeonggi-do (KR); Yeejin Jeon, Gyeonggi-do (KR); Wongyun Ahn, Gyeonggi-do (KR)

(21) Appl. No.: 18/694,432

(22) PCT Filed: Sep. 25, 2022
(86) PCT No.: PCT/EP2022/076603

§ 371 Date: Mar. 22, 2024

(30) Foreign Application Priority Data
Sep. 24, 2021 (EP) 21 199 013.0

(51) Int. Cl. A61K 31/439; A61K 31/4439; A61K 31/444; A61K 31/4709; A61K 31/496; A61K 31/497; A61K 31/506; A61K 31/5377; A61K 31/5386; A61P 35/00; C07D 498/08; C07D 519/00

(52) U.S. Cl. CPC A61K 31/439; A61K 31/4439; A61K 31/444; A61K 31/4709; A61K 31/496; A61K 31/497; A61K 31/506; A61K 31/5377; A61K 31/5386; A61P 35/00; C07D 498/08; C07D 519/00

(57) ABSTRACT

The present invention relates to certain macrocyclic compounds of the formula (I) and pharmaceutically acceptable salts thereof. These compounds are useful in the treatment or prevention of a disease associated with and/or caused by proteasome or immunoproteasome, selected from a cancer, an infectious disease, an inflammatory disease, and autoimmune disease.

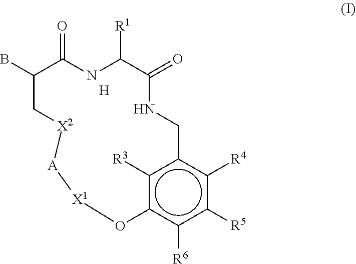

The present invention relates to certain macrocyclic compounds of the formula (I) and pharmaceutically acceptable salts thereof. These compounds are useful in the treatment or prevention of a disease treatable by proteasome inhibition, selected from cancers, infectious diseases, inflammatory diseases, and autoimmune diseases.

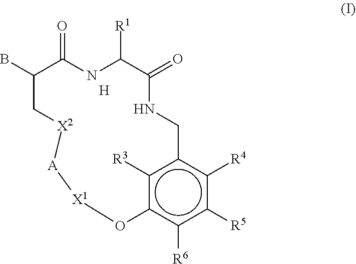

### BACKGROUND OF THE INVENTION

The proteasome is a multicatalytic proteinase complex with a highly ordered ring-shaped 20S core structure. The core structure is composed of 4 rings of 28 non-identical subunits; 2 rings are composed of 7 alpha subunits and 2 rings are composed of 7 beta subunits. Proteasomes are distributed throughout eukaryotic cells at a high concentration and cleave peptides in an ATP/ubiquitin-dependent process in a non-lysosomal pathway. Two isoforms of the proteasome are the constitutive proteasome and the immunoproteasome which is constitutively expressed in hematopoietic cells and can be induced in non-immune cells by inflammatory cytokines or oxidative stress. Both differ from each other in the subunit composition. The constitutive proteasome has three subunits with chymotrypsin-like (beta type-5), caspase-like (beta type-1) and trypsin-like (beta type-2) enzymatic activity. In the immunoproteasome these subunits are replaced by the beta type-5i, beta type-1i and beta type-2i catalytic subunits, which have different substrate preferences and an increased proteolytic activity compared to the corresponding subunits in the constitutive proteasome. An essential function of the immunoproteasome is the processing of class I MHC peptides for antigen presentation.

The proteasomes form a pivotal component for the ubiquitin-proteasome system (UPS) and corresponding cellular Protein Quality Control (PQC). Protein ubiquitination and subsequent proteolysis and degradation by the proteasome are important mechanisms in the regulation of the cell cycle, cell growth and differentiation, gene transcription, signal transduction and apoptosis.

The proteasome plays a central role in the cellular protein degradation pathway. Protein degradation is of particular importance for cancer cells with a high protein turnover, which is usually associated with the formation of misfolded proteins. Inhibition of the proteasome in such cells leads to an accumulation of misfolded proteins activating the caspase cascade and thus contributing to apoptosis. Alternative events induced by proteasome inhibition contributing to induce cell death are: Inhibition of the pro-survival NFkB pathway and induction of pro-apoptotic proteins. Thus proteasome inhibition leads to apoptosis of cells with a high protein turnover such as tumor and myeloma cells. Also plasma cells which have a high protein turnover due to the constant secretion of antibodies show sensitivity to proteasome inhibition. Accordingly proteasome inhibition has been shown to have a significant effect on autoantibody mediated autoimmune diseases such as systemic lupus erythematosus (SLE) or myasthenia gravis (MG).

Since proteasome inhibitors are effective at targeting antibody producing B-cells they are also evaluated as therapies in antibody-mediated allograft rejection. The inhibition of the protozoan proteasome by proteasome inhibitors has been shown to be effective in treating malaria, killing selectively Plasmodium falciparum while sparing human cells. In addition proteasome inhibitors have shown potential as antibiotics for example against Mycobacterium tuberculosis.

It is object to the present invention to provide novel compounds as proteasome subunit beta type-5 inhibitors and/or pharmaceutically acceptable salts thereof, which can be used as pharmaceutically active agents, and use thereof especially for prophylaxis and/or treatment of a disease associated with and/or caused by proteasome subunit beta type-5 selected from a cancer, a neurodegenerative disease, an infectious disease, and an inflammatory disease as well as compositions comprising at least one of those compounds and/or pharmaceutically acceptable salts thereof as pharmaceutically active ingredients. The objective of the present invention is solved by the teaching of the independent claims. Further advantageous features, aspects and details of the invention are evident from the dependent claims, the description, and the examples of the present application.

### DESCRIPTION OF THE INVENTION

The present invention relates to a compound of the general formula (I)

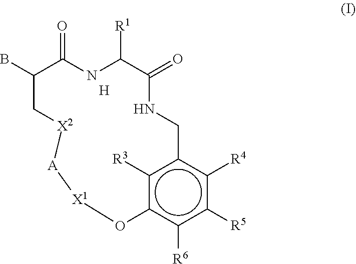

wherein
A represents —CO—N(R^{N6})—,

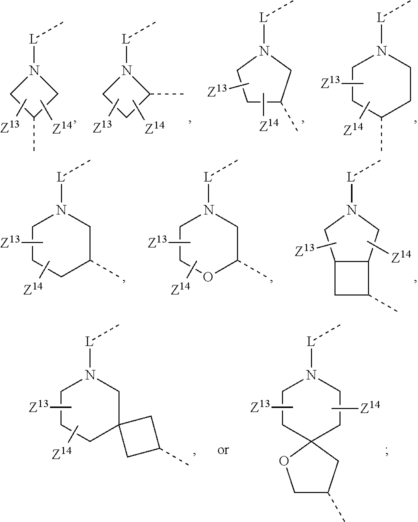

B represents —H, —NH(R²), —N(R²)(R^{N5}),

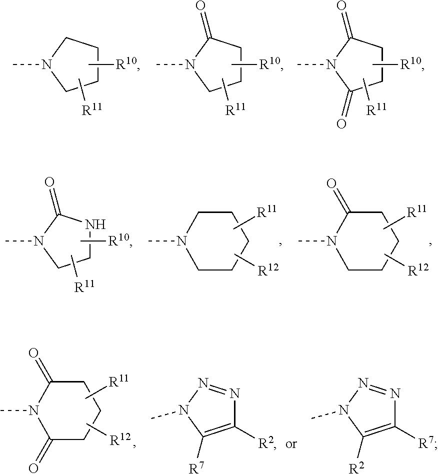

L represents —CO—, —CO—NH—, —CO—N(R^{N3})—, or —CO—O—;
R¹ represents —H, —(CH₂)ₚ—R⁷, —(CH₂)ₚ—NH—R⁷, —(CH₂)ₚ—R⁹, or —(CH₂)ₚ—NR^{N4}—R⁹;
R² represents —H, —R⁸, —R¹¹, -L¹-R¹¹, -L¹-(CH₂)ᵣ—R⁸, -L¹-R¹⁰, -L¹-(C₂H₄O)ₛ—R¹¹, -L¹-(CH₂)ₜ—O—R¹¹, -L¹-(CH₂)ₜ—NH—(CH₂)ᵣ—R⁸, -L¹-(CH₂)ₜ—O—(CH₂)ᵣ—R⁸, -L¹-(CH₂)ₜ—NHR⁸, -L¹-(CH₂)ₜ—NH—CO—R⁸, -L¹-(CH₂)ₜ—NH—SO₂—R⁸, -L¹-(CH₂)ₜ—NR^{N6}R¹⁰, -L¹-(CH₂)ₜ—O—(CH₂)ᵤ—NR^{N6}R¹⁰, -L¹-(CH₂)ᵣ—R¹⁴, —CO—C(R¹²)(R¹³)—R¹⁰, —CO—C(R¹²)(R¹³)—R⁸, or —CO—C(R¹²)(R¹³)—(CH₂)ᵤ—R⁸;
L¹ represents a bond, —CO—, —CO₂—, —CONH—, or —SO₂—;
R³-R⁶ represent independently of each other —H, —CH₃, —OCH₃, —F, or —Cl; or R⁵ and R⁶ may form together

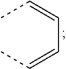

R⁸ and R⁹ represent independently of each other C₆-C₁₄ aryl, C₁-C₁₀ heteroaryl, C₃-C₈ carbocyclyl, C₁-C₉ heterocyclyl, C₄-C₁₁ bicyclic carbocyclyl, C₄-C₁₁ bridged carbocyclyl, C₁-C₁ bicyclic heterocyclyl, C₁-C₁₀ bridged heterocyclyl, C₇-C₁₆-spiroalkyl, C₅-C₁₄-spiroheterocyclyl,
wherein all afore-mentioned ring systems can be substituted with 1 to 5 substituents selected from Z¹, Z², Z³, Z⁴, Z⁵, Z⁶, Z⁷, Z⁸, Z⁹, Z¹⁰, Z¹¹, Z¹², R^{N1} and R^{N2}; and
C₁-C₁ heteroaryl, C₁-C₉ heterocyclyl, C₁-C₁ bicyclic heterocyclyl, C₁-C₁ bridged heterocyclyl, C₅-C₁₄-spiroheterocyclyl ring systems contain at least one of heteroatoms N, O, and S;
said C₃-C₈ carbocyclyl, C₁-C₉ heterocyclyl, C₁-C₁ bicyclic heterocyclyl, C₄-C₁₁ bridged carbocyclyl, C₁-C₁ bicyclic heterocyclyl, C₁-C₁ bridged heterocyclyl, C₇-C₁₆-spiroalkyl, C₅-C₁₄-spiroheterocyclyl ring systems can be partly saturated or unsaturated;
R⁷ and R¹⁰ represent independently of each other —H, —CH₃, —C₂H₅, —C₃H₇, —CH(CH₃)₂, —CH₂F, —CHF₂, —CF₃, —CH₂CN, —C(CH₃)₂—CN, —CH₂—C(CH₃)₂—CN, —CH₂—CF₃, —CH₂—C(CH₃)₂—NH₂,

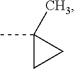

cyclo-C₃H₅, cyclo-C₄H₇, cyclo-C₅H₉, cyclo-C₆H₁₁, cyclo-C₇H₁₃, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, —C₇H₁₅, —C₈H₁₇, -Ph, —CH₂-Ph, —CH₂—CH₂-Ph, —C₂H₄—CH═CH₂, —CH₂—CH═CH—CH₃, —CH₂—C(CH₃)=CH₂, —CH(CH₃)—CH═CH₂, —C(CH₃)=CH—CH₃, —CH₂—CH═C(CH₃)₂, —CO—CH═C(CH₃)₂, —CH₂—C≡CH, —C₂H₄—C≡CH, —CH₂—C≡C—CH₃, —CH₂—OCF₃, —C₂H₄—OCF₃, —C₃H₆—OCF₃, —CH₂—OCHF₂, —C₂H₄—OCHF₂, —C₃H₆—OCHF₂, —CH₂—OCH₃, —C₂H₄—OCH₃, —C₃H₆—OCH₃, —CH₂—OC₂H₅, —C₂H₄—OC₂H₅, —C₃H₆—OC₂H₅, —CH₂—OH, —C₂H₄—OH, —C₃H₆—OH, —CH₂—COOH, —C₂H₄—COOH, —C₃H₆—COOH, —C(CH₃)₂—CN, —C(CH₃)₂—OH, —C(CH₃)₂—CH₂—OH, —C(C₂H₅)₂—CH₂—OH, —C(CH₂—OH)₂—CH₃, —C(CH₂—OH)₂—C₂H₅, —C(CH₃)₂—CH₂—SH, —C(C₂H₅)₂—CH₂—SH, —C(CH₂—SH)₂—CH₃, —CO—O—C(CH₃)₃, or —C(CH₂—SH)₂—C₂H₅;
R¹¹ represents —H, —CH₃, —C₂H₅, —C₃H₇, —CH(CH₃)₂, —CH₂F, —CHF₂, —CF₃, —CH₂CN, —C(CH₃)₂—CN, —CH₂—C(CH₃)₂—CN, —CH₂—CF₃, —CH₂—C(CH₃)₂—H₃

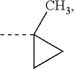

cyclo-C₃H₅, cyclo-C₄H₇, cyclo-C₅H₉, cyclo-C₆H₁₁, cyclo-C₇H₁₃, —C₄H₉, —CH₂—CH(CH₃)₂, —CH(CH₃)—C₂H₅, —C(CH₃)₃, —CH₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, —C₇H₁₅, —C₈H₁₇, -Ph, —CH₂-Ph, —CH₂—CH₂-Ph, —CH═CH₂, —CH₂—CH═CH₂, —C(CH₃)=CH₂, —CH═CH—CH₃, —C(CH₃)=CH—CH₃, —CH═C(CH₃)₂, —C(CH₃)═C(CH₃)₂, —C₂H₄—CH═CH₂, —CH₂—CH═CH—CH₃, —CH₂—C(CH₃)=CH₂, —CH(CH₃)—CH═CH₂, —C(CH₃)=CH—CH₃, —CH₂—CH═C(CH₃)₂, —C≡CH, —CH₂—C≡CH, —C₂H₄—C≡CH, —CH₂—C≡C—CH₃, —C≡C—CH₃, —C≡C—C₂H₅, —CH₂—OCF₃, —C₂H₄—OCF₃, —C₃H₆—OCF₃, —CH₂—OCHF₂, —C₂H₄—OCHF₂, —C₃H₆—OCHF₂, —CH₂—OCH₃, —C₂H₄—OCH₃, —C₃H₆—OCH₃, —CH₂—OC₂H₅, —C₂H₄—OC₂H₅, —C₃H₆—OC₂H₅, —CH₂—OH, —C₂H₄—OH, —C₃H₆—OH, —C(CH₃)₂—CH₂—OH, —C(C₂H₅)₂—CH₂—OH, —C(CH₂—OH)₂—CH₃, —C(CH₂—OH)₂—C₂H₅, —C(CH₃)₂—CH₂—SH, —C(C₂H₅)₂—CH₂—SH, —C(CH₂—SH)₂—CH₃, or —C(CH₂—SH)₂—C₂H₅;
R¹² and R¹³ represent independently of each other —H, —CH₃, —C₂H₅, —C₃H₇, —CH(CH₃)₂, -Ph, —CH₂-Ph, —COOH, —NH₂, —NHCO₂(CCH₃)₃, —CH₂—NH₂, —CHF₂, —F, —CF₃, —OCF₃, —OCHF₂, —OH, —OCH₃, —OC₂H₅, —OC₃H₇, and —OCH(CH₃)₂; or
R² and R¹³ may form together

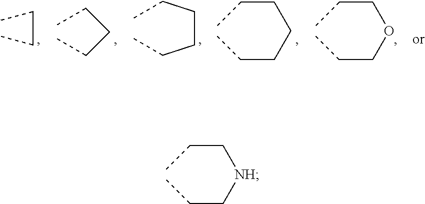

R¹⁴ represents

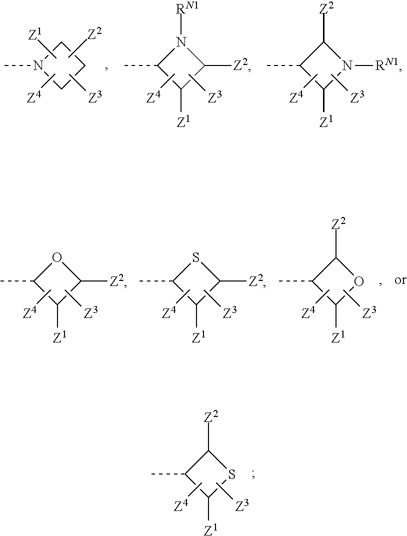

R¹⁵ and R¹⁶ represent independently of each other —X³-L²-R¹⁷, or —(OCH₂CH₂)_{w}—R¹⁷;
L² represents —(CH₂)ᵥ—, —(CH₂CH₂—O)_{w}—CH₂—, or —(CH₂CH₂—O)_{w}—CH₂CH₂—;
R¹⁷ represents —OH, —SH, —SO₃H, —NH₂, or —CO₂H;
R^{N1}, R^{N2}, R^{N3} and R^{N4} represent independently of each other —H, —CH₃, —C₂H₅, —C₃H₇, —CH(CH₃)₂, —CHF₂, —CF₃,

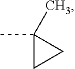

cyclo-C₃H₅, cyclo-C₄H₇, cyclo-C₅H₉, cyclo-C₆H₁₁, cyclo-C₇H₁₃, —C₄H₉, —CH₂—CH(CH₃)₂, —CH(CH₃)—C₂H₅, —C(CH₃)₃, —CH₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, —C₇H₁₅, —C₈H₁₇, -Ph, —CH₂-Ph, —CH₂—CH₂-Ph, —C₂H₄—CH═CH₂, —CH₂—CH═CH—CH₃, —CH₂—C(CH₃)=CH₂, —CH(CH₃)—CH═CH₂, —CH₂—CH═C(CH₃)₂, —CH₂—C≡CH, —C₂H₄—C≡CH, —CH₂—C≡C—CH₃, —COCH₃, —COC₂H₅, —COC₃H₇, —CO-cyclo-C₃H₅, —COCH(CH₃)₂, —COC(CH₃)₃, —COOH, —COOCH₃, —COOC₂H₅, —COOC₃H₇, —COO-cyclo-C₃H₅, —COOCH(CH₃)₂, —COOC(CH₃)₃, —COOCH₂Ph, —CONH₂, —CONHCH₃, —CONHC₂H₅, —CONHC₃H₇, —CONH-cyclo-C₃H₅, —CONH[CH(CH₃)₂], —CONH[C(CH₃)₃], —CON(CH₃)₂, —CON(C₂H₅)₂, —CON(C₃H₇)₂, —CON(cyclo-C₃H₅)₂, —CON[CH(CH₃)₂]₂, —CON[C(CH₃)₃]₂, —SO₃H, or —R¹⁵;
R^{N5} and R^{N6} represent independently of each other —H, —CH₃, —C₂H₅, —C₃H₇, —CH(CH₃)₂, cyclo-C₃H₅, —COOC(CH₃)₃, or —COOCH₂Ph;
X¹ represents —(CH₂)ₘ—;
X² represents —(CH₂)ₙ—;
X³ represents a bond, —O—, —NH—, or —S—;
Z¹-Z¹⁴ represent independently of each other

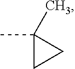

cyclo-C₃H₅, cyclo-C₄H₇, cyclo-C₅H₉, cyclo-C₆H₁₁, cyclo-C₇H₁₃, —R¹⁶, —H, —OH, —OCH₃, —OC₂H₅, —OC₃H₇, —O-cyclo-C₃H₅, —OCH(CH₃)₂, —OC(CH₃)₃, —OC₄H₉, —O-cyclo-C₄H₇, —O-cyclo-C₅H₉, —O-cyclo-C₆H₁₁, —OCH₂CH(CH₃)₂, —OCH₂-cyclo-C₃H₅, —OCH₂-cyclo-C₄H₇, —OCH₂—Cyclo-C₅H₉, —OCH₂-cyclo-C₆H₁₁, —OPh, —OCH₂-Ph, —OCPh₃, —CH₂—OCH₃, —C₂H₄—OCH₃, —C₃H₆—OCH₃, —CH₂—OC₂H₅, —C₂H₄—OC₂H₅, —C₃H₆—OC₂H₅, —CH₂—OC₃H₇, —C₂H₄—OC₃H₇, —C₃H₆—OC₃H₇, —CH₂—O-cyclo-C₃H₅, —C₂H₄—O-cyclo-C₃H₅, —C₃H₆—O-cyclo-C₃H₅, —CH₂—OCH(CH₃)₂, —C₂H₄—OCH(CH₃)₂, —C₃H₆—OCH(CH₃)₂, —CH₂—OC(CH₃)₃, —C₂H₄—OC(CH₃)₃, —C₃H₆—OC(CH₃)₃, —CH₂—OC₄H₉, —C₂H₄—OC₄H₉, —C₃H₆—OC₄H₉, —CH₂—OPh, —C₂H₄—OPh, —C₃H₆—OPh, —CH₂—OCH₂-Ph, —C₂H₄—OCH₂-Ph, —C₃H₆—OCH₂-Ph, —SH, —SCH₃, —SC₂H₅, —SC₃H₇, —S-cyclo-C₃H₅, —SCH(CH₃)₂, —SC(CH₃)₃, —F, —Cl, —Br, —I, —CN, —COCH₃, —COC₂H₅, —COC₃H₇, —CO-cyclo-C₃H₅, —COCH(CH₃)₂, —COC(CH₃)₃, —COOH, —COOCH₃, —COOC₂H₅, —COOC₃H₇, —COO-cyclo-C₃H₅, —COOCH(CH₃)₂, —COOC(CH₃)₃, —OOC—CH₃, —OOC—C₂H₅, —OOC—C₃H₇, —OOC-cyclo-C₃H₅, —OOC—CH(CH₃)₂, —OOC—C(CH₃)₃, —CONH₂, —CONHCH₃, —CONHC₂H₅, —CONHC₃H₇, —CONH-cyclo-C₃H₅, —CONH[CH(CH₃)₂], —CONH[C(CH₃)₃], —CON(CH₃)₂, —CON(C₂H₅)₂, —CON(C₃H₇)₂, —CON(cyclo-C₃H₅)₂, —CON[CH(CH₃)₂]₂, —CON[C(CH₃)₃]₂, —NHCOCH₃, —NHCOC₂H₅, —NHCOC₃H₇, —NHCO-cyclo-C₃H₅, —NHCO—CH(CH₃)₂, —NHCO—C(CH₃)₃, —NHCO—CH(NH₂)CH₂—COOH, —NHCO—CH(NH₂)CH₂CH₂—COOH, —NHCO—OCH₃, —NHCO—OC₂H₅, —NHCO—OC₃H₇, —NHCO—O-cyclo-C₃H₅, —NHCO—OCH(CH₃)₂, —NHCO—OC(CH₃)₃, —NH₂, —NHCH₃, —NHC₂H₅, —NHC₃H₇, —NH-cyclo-C₃H₅, —NHCH(CH₃)₂, —NHC(CH₃)₃, —N(CH₃)₂, —N(C₂H₅)₂, —N(C₃H₇)₂, —N(cyclo-C₃H₅)₂, —N[CH(CH₃)₂]₂, —N[C(CH₃)₃]₂, —SOCH₃, —SOC₂H₅, —SOC₃H₇, —SO-cyclo-C₃H₅, —SOCH(CH₃)₂, —SOC(CH₃)₃, —SO₂CH₃, —SO₂C₂H₅, —SO₂C₃H₇, —SO₂-cyclo-C₃H₅, —SO₂CH(CH₃)₂, —SO₂C(CH₃)₃, —SO₃H, —SO₃CH₃, —SO₃C₂H₅, —SO₃C₃H₇, —SO₃-cyclo-C₃H₅, —SO₃CH(CH₃)₂, —SO₃C(CH₃)₃, —SO₂NH₂, —SO₂NHCH₃, —SO₂NHC₂H₅, —SO₂NHC₃H₇, —SO₂NH-cycl-C₃H₅, —SO₂NHCH(CH₃)₂, —SO₂NHC(CH₃)₃, —SO₂N(CH₃)₂, —SO₂N(C₂H₅)₂, —SO₂N(C₃H₇)₂, —SO₂N(cyclo-C₃H₅)₂, —SO₂N[CH(CH₃)₂]₂, —SO₂N[C(CH₃)₃]₂, —O—S(═O)CH₃, —O—S(═O)C₂H₅, —O—S(═O)C₃H₇, —O—S(═O)-cyclo-C₃H₅, —O—S(═O)CH(CH₃)₂, —O—S(═O)C(CH₃)₃, —S(═O)(═NH)CH₃, —S(═O)(═NH)C₂H₅, —S(═O)(═NH)C₃H₇, —S(═O)(═NH)-cyclo-C₃H₅, —S(═O)(═NH)CH(CH₃)₂, —S(═O)(═NH)C(CH₃)₃, —NH—SO₂—CH₃, —NH—SO₂—C₂H₅, —NH—SO₂—C₃H₇, —NH—SO₂-cyclo-C₃H₅, —NH—SO₂—CH(CH₃)₂, —NH—SO₂—C(CH₃)₃, —O—SO₂—CH₃, —O—SO₂—C₂H₅, —O—SO₂—C₃H₇, —O—SO₂-cyclo-C₃H₅, —O—SO₂—CH(CH₃)₂, —O—SO₂—C(CH₃)₃, —OCH₂F, —OCHF₂, —OCF₃, —CH₂—OCF₃, —C₂H₄—OCF₃, —C₃H₆—OCF₃, —CH₂—OCHF₂, —C₂H₄—OCHF₂, —C₃H₆—OCHF₂, —OC₂F₅, —CH₂—OC₂F₅, —C₂H₄—OC₂F₅, —C₃H₆—OC₂F₅, —O—COOCH₃, —O—COOC₂H₅, —O—COOC₃H₇, —O—COO-cyclo-C₃H₅, —O—COOCH(CH₃)₂, —O—COOC(CH₃)₃, —NH—CO—NH₂, —NH—CO—NHCH₃, —NH—CO—NHC₂H₅, —NH—CO—NHC₃H₇, —NH—C(═NH)—NH₂, —NH—CO—N(C₃H₇)₂, —NH—CO—NH[CH(CH₃)₂], —NH—CO—NH[C(CH₃)₃], —NH—CO—N(CH₃)₂, —NH—CO—N(C₂H₅)₂, —NH—CO—NH-cyclo-C₃H₅, —NH—CO—N(cyclo-C₃H₅)₂, —NH—CO—N[CH(CH₃)₂]₂, —NH—C(═NH)—NHCH₃, —NH—C(═NH)—NHC₂H₅, —NH—C(═NH)—NHC₃H₇, —O—CO—NH-cyclo-C₃H₅, —NH—C(═NH)—NH-cyclo-C₃H₅, —NH—C(═NH)—NH[CH(CH₃)₂], —O—CO—NH[CH(CH₃)₂], —NH—C(═NH)—NH[C(CH₃)₃], —NH—C(═NH)—N(CH₃)₂, —NH—C(═NH)—N(C₂H₅)₂, —NH—C(═NH)—N(C₃H₇)₂, —NH—C(═NH)—N(cyclo-C₃H₅)₂, —O—CO—NHC₃H₇, —NH—C(═NH)—N[CH(CH₃)₂]₂, —NH—C(═NH)—N[C(CH₃)₃]₂, —O—CO—NH₂, —O—CO—NHCH₃, —O—CO—NHC₂H₅, —O—CO—NH[C(CH₃)₃], —O—CO—N(CH₃)₂, —O—CO—N(C₂H₅)₂, —O—CO—N(C₃H₇)₂, —O—CO—N(cyclo-C₃H₅)₂, —O—CO—N[CH(CH₃)₂]₂, —O—CO—N[C(CH₃)₃]₂, —O—CO—OCH₃, —O—CO—OC₂H₅, —O—CO—OC₃H₇, —O—CO—O-cyclo-C₃H₅, —O—CO—OCH(CH₃)₂, —O—CO—OC(CH₃)₃, —CH₂F, —CHF₂, —CF₃, —CH₂—CH₂F, —CH₂—CHF₂, —CH₂—CF₃, cyclo-C₈H₁₅, -Ph, —CH₂-Ph, —CH₂—CH₂-Ph, —CH═CH-Ph, —CPh₃, —CH₃, —C₂H₅, —C₃H₇, —CH(CH₃)₂, —C₄H₉, —CH₂—CH(CH₃)₂, —CH(CH₃)—C₂H₅, —C(CH₃)₃, —CH₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, —C₇H₁₅, —C₈H₁₇, —C₃H₆—CH(CH₃)₂, —C₂H₄—CH(CH₃)—C₂H₅, —CH(CH₃)—C₄H₉, —CH₂—CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, —CH(CH₃)—C(CH₃)₃, —CH═CH₂, —CH₂—CH═CH₂, —C(CH₃)=CH₂, —CH═CH—CH₃, —C₂H₄—CH═CH₂, —CH₂—CH═CH—CH₃, —CH═CH—C₂H₅, —CH₂—C(CH₃)=CH₂, —CH(CH₃)—CH═CH, —CH═C(CH₃)₂, —C(CH₃)=CH—CH₃, —CH═CH—CH═CH₂, —C₃H₆—CH═CH₂, —C₂H₄—CH═CH—CH₃, —CH₂—CH═CH—C₂H₅, —CH═CH—C₃H₇, —CH═CH—CH═CH—CH₃, —C₂H₄—C(CH₃)=CH₂, —CH₂—CH(CH₃)—CH═CH₂, —CH(CH₃)—CH₂—CH═CH₂, —CH₂—CH═C(CH₃)₂, —CH₂—C(CH₃)=CH—CH₃, —CH(CH₃)—CH═CH—CH₃, —CH═CH—CH(CH₃)₂, —CH═C(CH₃)—C₂H₅, —C(CH₃)=CH—C₂H₅, —C(CH₃)═C(CH₃)₂, —C(CH₃)₂—CH═CH₂, —CH(CH₃)—C(CH₃)=CH₂, —C₄H₈—CH═CH₂, —C₃H₆—CH═CH—CH₃, —C₂H₄—CH═CH—C₂H₅, —CH₂—CH═CH—C₃H₇, —CH═CH—C₄H₉, —C₃H₆—C(CH₃)=CH₂, —C₂H₄—CH(CH₃)—CH═CH₂, —CH₂—CH(CH₃)—CH₂—CH═CH₂, —C₂H₄—CH═C(CH₃)₂, —CH(CH₃)—C₂H₄—CH═CH₂, —C₂H₄—C(CH₃)=CH—CH₃, —CH₂—CH(CH₃)—CH═CH—CH₃, —CH(CH₃)—CH₂—CH═CH—CH₃, —CH₂—CH═CH—CH(CH₃)₂, —CH₂—CH═C(CH₃)—C₂H₅, —CH₂—C(CH₃)=CH—C₂H₅, —CH(CH₃)—CH═CH—C₂H₅, —CH═CH—CH₂—CH(CH₃)₂, —CH═CH—CH(CH₃)—C₂H₅, —CH═C(CH₃)—C₃H₇, —C(CH₃)=CH—C₃H₇, —CH₂—CH(CH₃)—C(CH₃)=CH₂, —C[C(CH₃)₃]=CH₂, —CH(CH₃)—CH₂—C(CH₃)=CH₂, —CH(CH₃)—CH(CH₃)—CH═CH₂, —CH═CH—C₂H₄—CH═CH₂, —C(CH₃)₂—CH₂—CH═CH₂, —CH₂—C(CH₃)═C(CH₃)₂, —CH(CH₃)—CH═C(CH₃)₂, —C(CH₃)₂—CH═CH—CH₃, —CH═CH—CH₂—CH═CH—CH₃, —CH(CH₃)—C(CH₃)=CH—CH₃, —CH═C(CH₃)—CH(CH₃)₂, —C(CH₃)=CH—CH(CH₃)₂, —C(CH₃)═C(CH₃)—C₂H₅, —CH═CH—C(CH₃)₃, —C(CH₃)₂—C(CH₃)=CH₂, —CH(C₂H₅)—C(CH₃)=CH₂, —C(CH₃)(C₂H₅)—CH═CH₂, —CH(CH₃)—C(C₂H₅)=CH₂, —CH₂—C(C₃H₇)=CH₂, —CH₂—C(C₂H₅)=CH—CH₃, —CH(C₂H₅)—CH═CH—CH₃, —C(C₄H₉)=CH₂, —C(C₃H₇)=CH—CH₃, —C(C₂H₅)=CH—C₂H₅, —C(C₂H₅)═C(CH₃)₂, —C[CH(CH₃)(C₂H₅)]=CH₂, —C[CH₂—CH(CH₃)₂]=CH₂, —C₂H₄—CH═CH—CH═CH₂, —CH₂—CH═CH—CH₂—CH═CH₂, —C₃H₆—C≡C—CH₃, —CH₂—CH═CH—CH═CH—CH₃, —CH═CH—CH═CH—C₂H₅, —CH(CH₃)—CH₂—C≡CH, —CH(CH₃)—C≡C—CH₃, —C₂H₄—CH(CH₃)—C≡CH, —CH═CH—CH═C(CH₃)₂, —CH₂—CH(CH₃)—CH₂—C≡CH, —CH═CH—C(CH₃)=CH—CH₃, —CH═C(CH₃)—CH═CH—CH₃, —CH₂—CH(CH₃)—C≡CH, —C(CH₃)=CH—CH═CH—CH₃, —C≡CH, —C≡C—CH₃, —CH₂—C≡CH, —C₂H₄—C≡CH, —CH₂—C≡C—CH₃, —C≡C—C₂H₅, —C₃H₆—C≡CH, —C₂H₄—C≡C—CH₃, —CH₂—C≡C—C₂H₅, —C≡C—C₃H₇, —CH(CH₃)—C≡CH, —C₄H₈—C≡CH, —C₂H₄—C≡C—C₂H₅, —CH₂—C≡C—C₃H₇, —C≡C—C₄H₉, —C≡C—CH₂—CH(CH₃)₂, —CH(CH₃)—C₂H₄—C≡CH, —CH₂—CH(CH₃)—C≡C—CH₃, —C(CH₃)(C₂H₅)—C≡CH, —CH(CH₃)—CH₂—C≡C—CH₃, —CH(CH₃)—C≡C—C₂H₅, —CH₂—C≡C—CH(CH₃)₂, —C≡C—CH(CH₃)—C₂H₅, —CH₂—C≡C—C≡C—CH₃, —CH(C₂H₅)—C≡C—CH₃, —C(CH₃)₂—C≡C—CH₃, —CH(C₂H₅)—CH₂—C≡CH, —CH₂—CH(C₂H₅)—C≡CH, —C(CH₃)₂—CH₂—C≡CH, —CH₂—C(CH₃)₂—C≡CH, —CH(CH₃)—CH(CH₃)—C≡CH, —CH(C₃H₇)—C≡CH, —CH₂—CH(C≡CH)₂, —C≡C—C≡CH, —CH₂—C≡C—C≡CH, —C≡C—C≡C—CH₃, —CH(C≡CH)₂, —C₂H₄—C≡C—C≡CH, —CH₂—C≡C—CH₂—C≡CH, —C≡C—C₂H₄—C≡CH, —C≡C—C(CH₃)₃, —C≡C—CH₂—C≡C—CH₃, —C≡C—C≡C—C₂H₅,

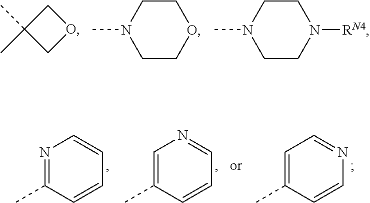

Z³ and Z⁴ may form together

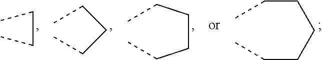

Z¹³ and Z¹⁴ may form together

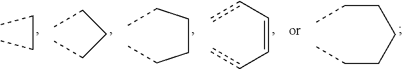

m is an integer selected from 0, 1, 2, 3, 4, 5, or 6;
n is an integer selected from 0, 1, 2, 3, 4, 5, or 6;
p is an integer selected from 0, 1, 2, 3, 4, 5, or 6;
r is an integer selected from 0, 1, 2, 3, or 4;
s is an integer selected from 1, 2, 3, 4, 5, 6, 7, 8, 9, or 10;
t is an integer selected from 1, 2, 3, or 4;
u is an integer selected from 1, 2, 3, or 4;
v is an integer selected from 0, 1, 2, 3, 4, 5, or 6;
w is an integer selected from 0, 1, 2, or 3;
   or an enantiomer, a stereoisomeric form, a mixture of enantiomers, a diastereomer, a mixture of diastereomers, a hydrate, a solvate, an acid salt form, a tautomer, a racemate of the above mentioned compounds, or a pharmaceutically acceptable salt thereof.

The term “pharmaceutically acceptable salt” refers to a salt of a compound that does not cause significant irritation to an organism to which it is administered and does not abrogate the biological activity and properties of the compound. The compounds of the present invention may form salts with organic or inorganic acids or bases. Examples of suitable acids for such acid addition salt formation are hydrochloric acid, hydrobromic acid, sulfuric acid, phosphoric acid, acetic acid, citric acid, oxalic acid, malonic acid, salicylic acid, p-aminosalicylic acid, malic acid, fumaric acid, succinic acid, ascorbic acid, maleic acid, sulfonic acid, phosphonic acid, perchloric acid, nitric acid, formic acid, propionic acid, gluconic acid, lactic acid, tartaric acid, hydroxymaleic acid, pyruvic acid, phenylacetic acid, benzoic acid, p-aminobenzoic acid, p-hydroxybenzoic acid, methanesulfonic acid, ethanesulfonic acid, nitrous acid, hydroxyethanesulfonic acid, ethylenesulfonic acid, p-toluenesulfonic acid, naphthylsulfonic acid, sulfanilic acid, camphorsulfonic acid, china acid, mandelic acid, o-methylmandelic acid, hydrogen-benzenesulfonic acid, picric acid, adipic acid, D-o-tolyltartaric acid, tartronic acid, (o, m, p)-toluic acid, naphthylamine sulfonic acid, trifluoroacetic acid, and other mineral or carboxylic acids well known to those skilled in the art. The salts are prepared by contacting the free base form of the compounds of formula (I) with a sufficient amount of the desired acid to produce a salt in the conventional manner well known to those skilled in the art.

In the case the inventive compounds bear acidic groups, salts could also be formed with inorganic or organic bases. Examples for suitable inorganic or organic bases are, for example, NaOH, KOH, NH₄OH, tetraalkylammonium hydroxide, lysine or arginine and the like. Salts may be prepared in a conventional manner using methods well known in the art, for example by treatment of a solution of the compound of the general formula (I) with a solution of an acid, selected out of the group mentioned above.

As used herein, the term “C₆-C₁₄-aryl” refers to aromatic residues or more specific to aromatic carbocyclic residues with one, two or three aromatic rings and refers preferably to phenyl and naphthyl, wherein these phenyl and naphthyl residues can be substituted with 1 to 5 substituents selected from Z¹ to Z¹². However it is clear to a skilled person that the term “can be substituted” refers to the replacement of a hydrogen atom by one of the substituents Z¹ to Z¹². The carbon atom number of C₆-C₁₄ refers only to the carbon atoms of the aromatic ring system (aryl) and does not include the carbon atoms of the substituents Z¹ to Z¹².

Examples of preferred C₆-C₁₄-aryl groups and substituted C₆-C₁₄-aryl residues are

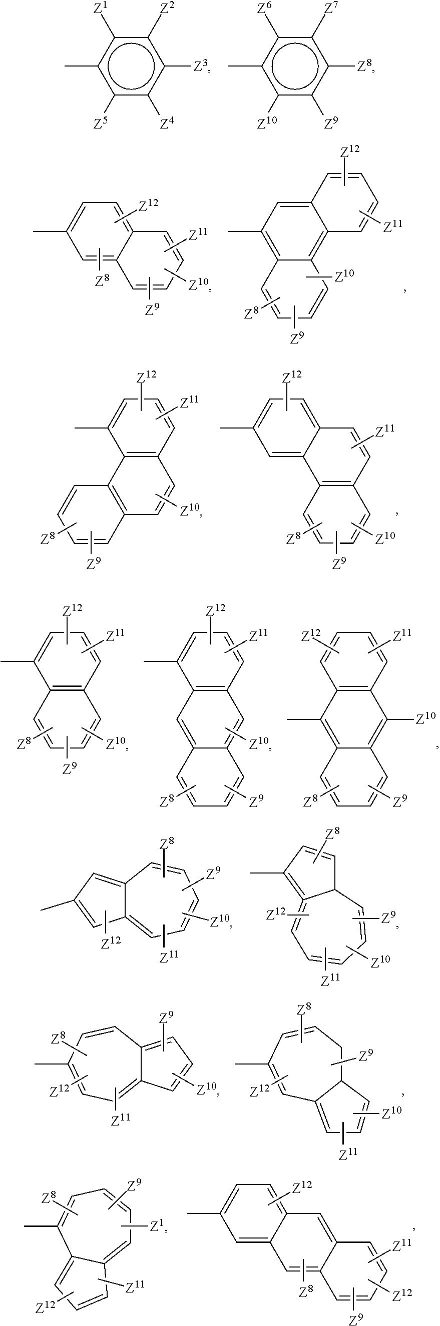

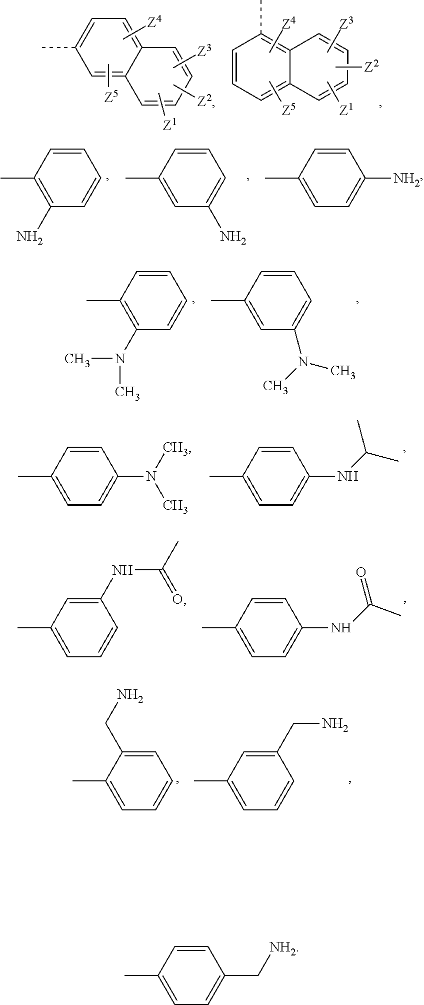

As used herein, the term “C₁-C₁₀-heteroaryl” refers to aromatic residues with one or more heteroatoms such as O, S, N and especially N and refers preferably to

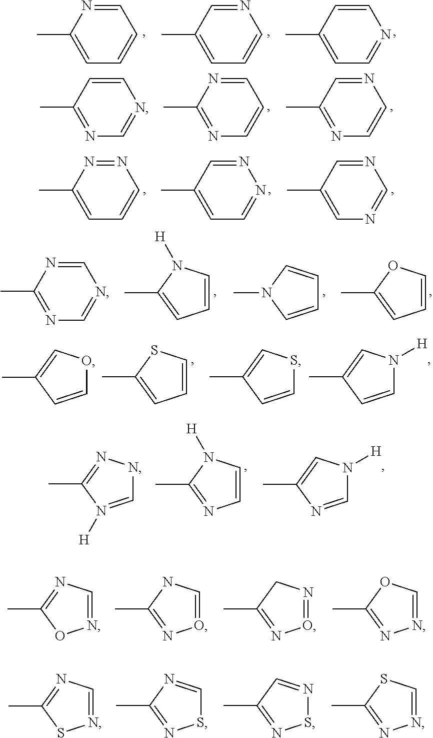

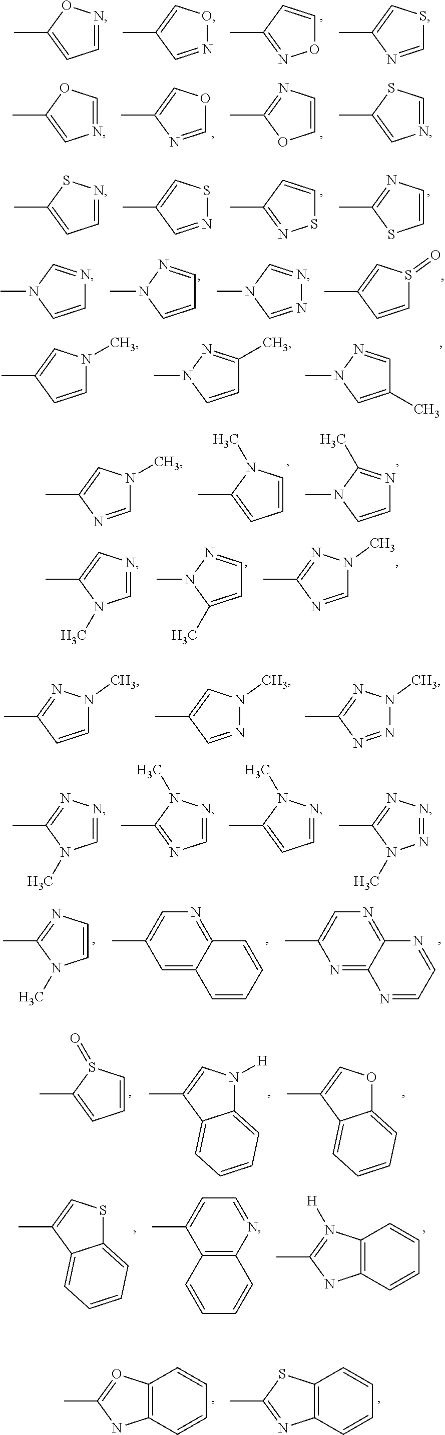

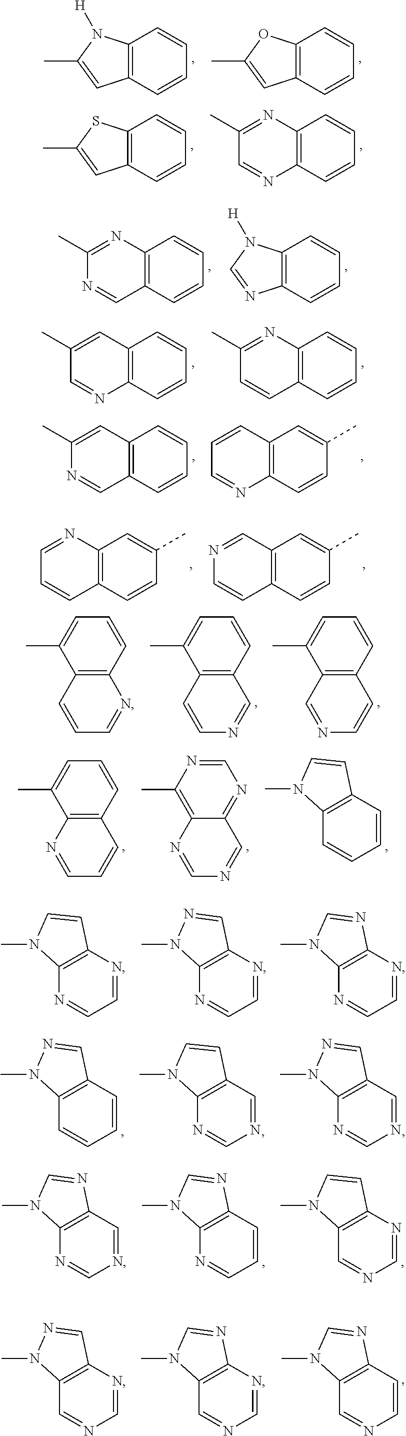

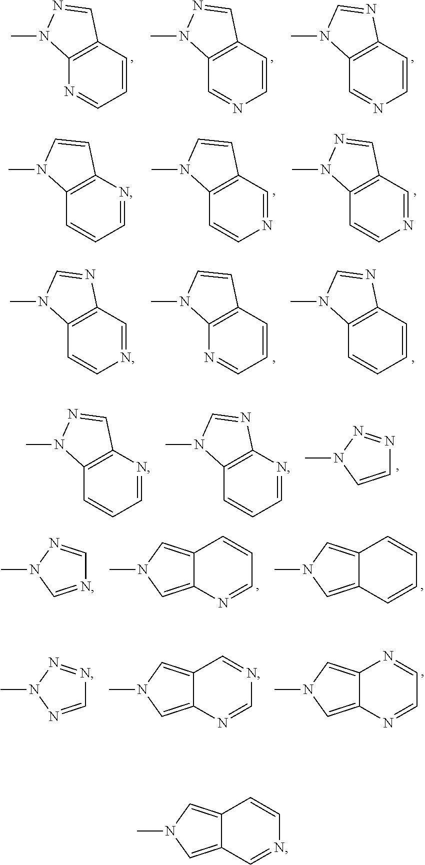

wherein these residues can be substituted with 1 to 5 substituents selected from R^{N1}, R^{N2}, Z¹ to Z¹². However it is clear to a skilled person that the term “can be substituted” refers to the replacement of a hydrogen atom by one of the substituents R^{N1}, R^{N2}, Z¹ to Z¹². Moreover it is clear to a skilled person that only these hydrogen atoms which are present in the residue can be replaced by the substituents R^{N1}, R^{N2}, Z¹ to Z¹². In case of secondary amine group in these heteroaryl residues, a hydrogen atom of secondary amine group is replaced by the substituent R^{N1} or R^{N2}. Thus, since the oxadiazole group has only one hydrogen atom, only one hydrogen atom can be replaced by one substituent selected from Z¹ to Z¹². The carbon atom number of C₁-C₁₀ refers only to the carbon atoms of the heteroaromatic ring system (heteroaryl) and does not include the carbon atoms of the substituents R^{N1}, R^{N2}, Z¹ to Z¹².

Examples of preferred substituted C₁-C₁₀-heteroaryl residues are

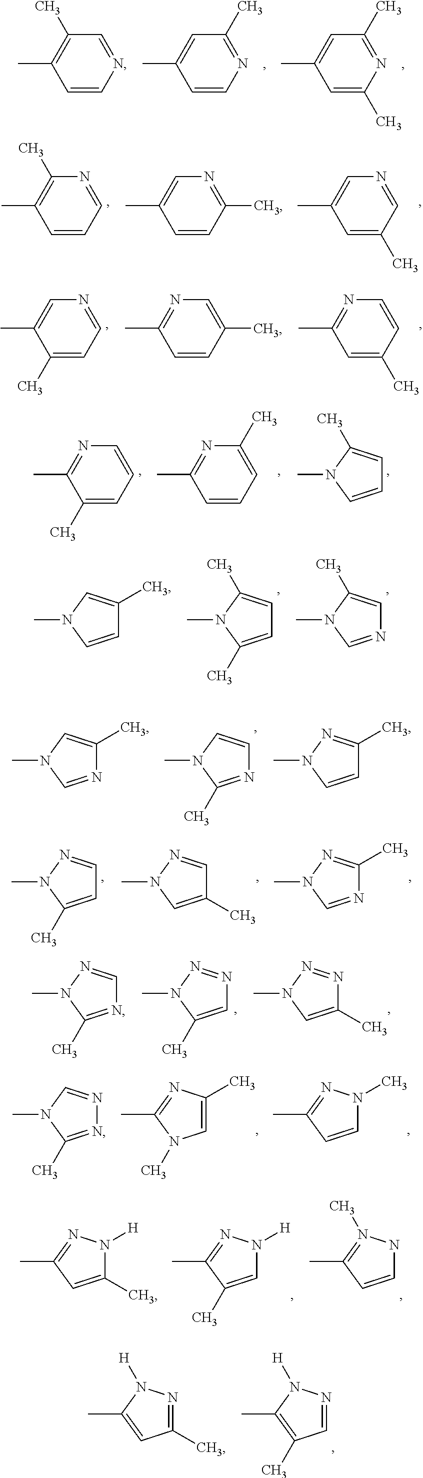

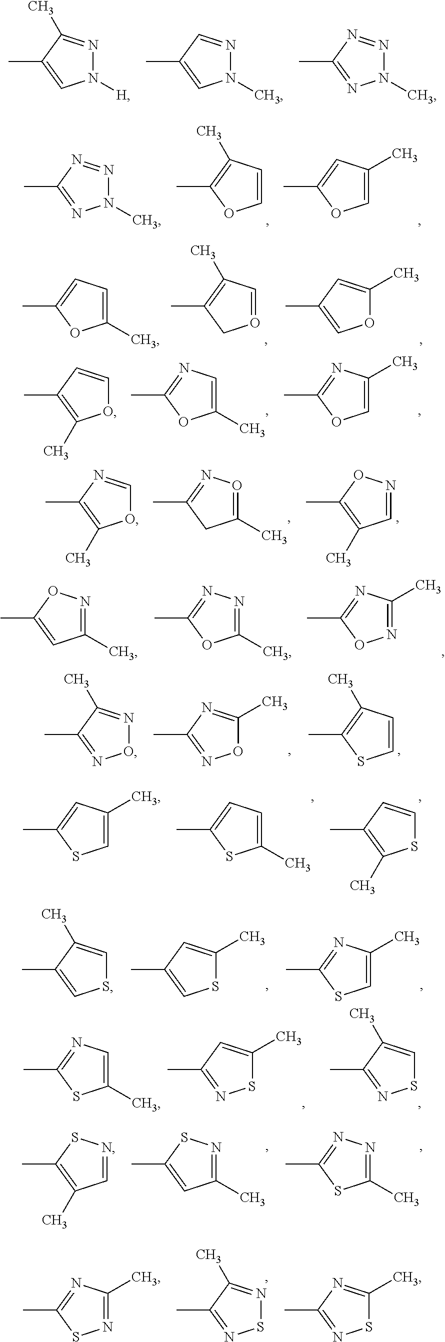

As used herein, C₃-C₈-carbocyclyl refers to cyclo-C₃H₅, cyclo-C₄H₇, cyclo-C₅H₉, cyclo-C₆H₁₁, cyclo-C₇H₁₃, and cyclo-C₈H₁₅, wherein these residues can be substituted with 1 to 5 substituents selected from Z¹ to Z¹², preferably, Z¹, Z², Z³, Z⁴ and Z⁵. However it is clear to a skilled person that the term “can be substituted” refers to the replacement of a hydrogen atom by one of the substituents Z¹ to Z¹², preferably, Z¹, Z², Z³, Z⁴ and Z⁵. The carbon atom number of C₃-C₈ refers only to the carbon atoms of the cycloalkyl residue and does not include the carbon atoms of the substituents Z¹ to Z¹².

Examples of preferred substituted C₃-C₈-cycloalkyl residues are

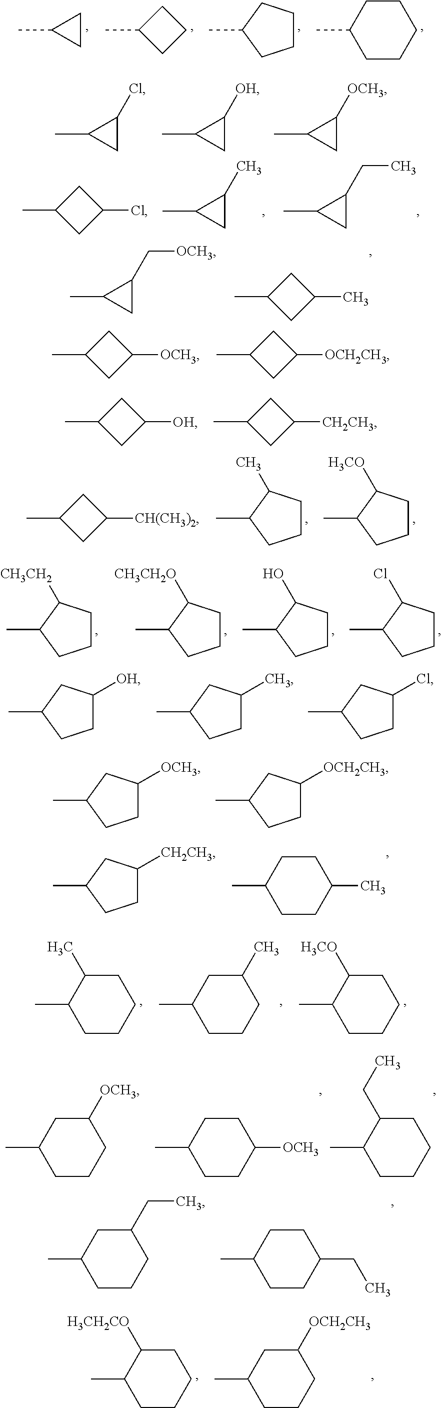

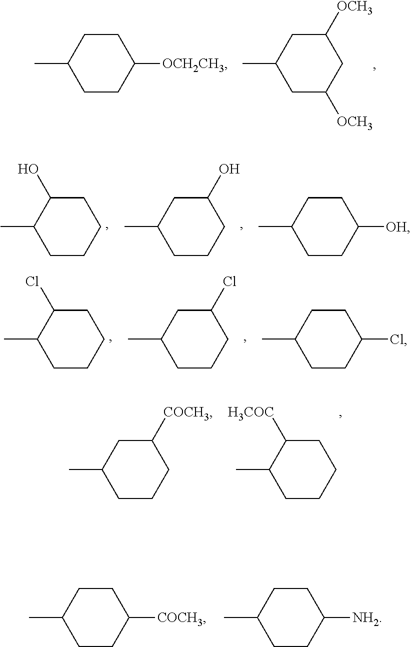

As used herein, the term “C₁-C₉-heterocyclyl” covers saturated or partly unsaturated heterocyclic residues with 1 to 9 ring carbon atoms, but not aromatic residues and covers also bicyclic saturated or partly unsaturated residues with 1 to 9 ring carbon atoms, but preferably not fully aromatic residues which are aromatic throughout the bicyclic system but may comprise partly aromatic ring systems, wherein one ring of the bicyclic ring system is aromatic.

Examples of preferred substituted C₁-C₉-heterocyclyl residues are

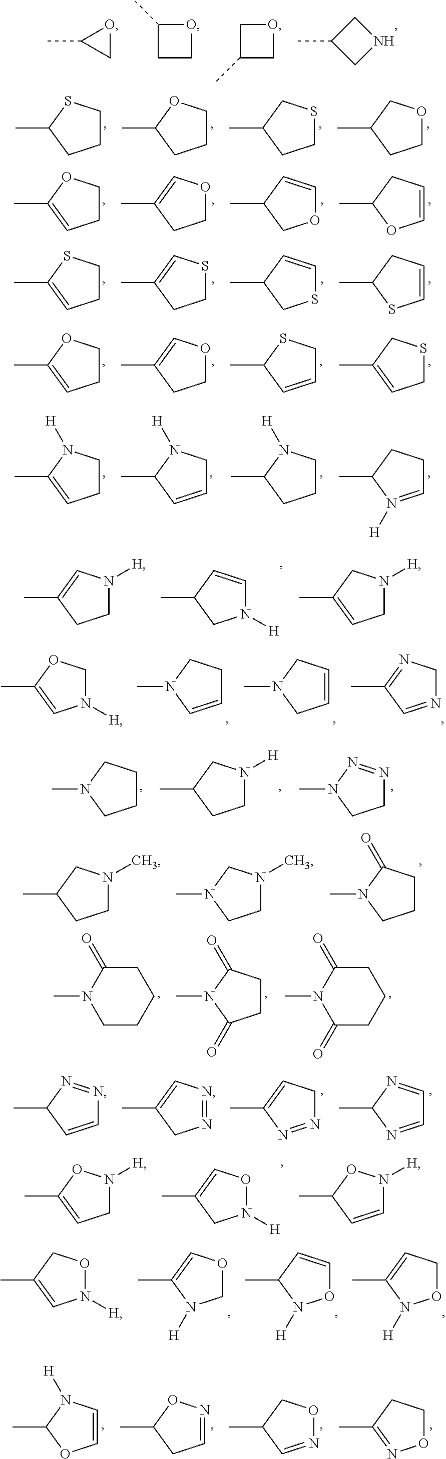

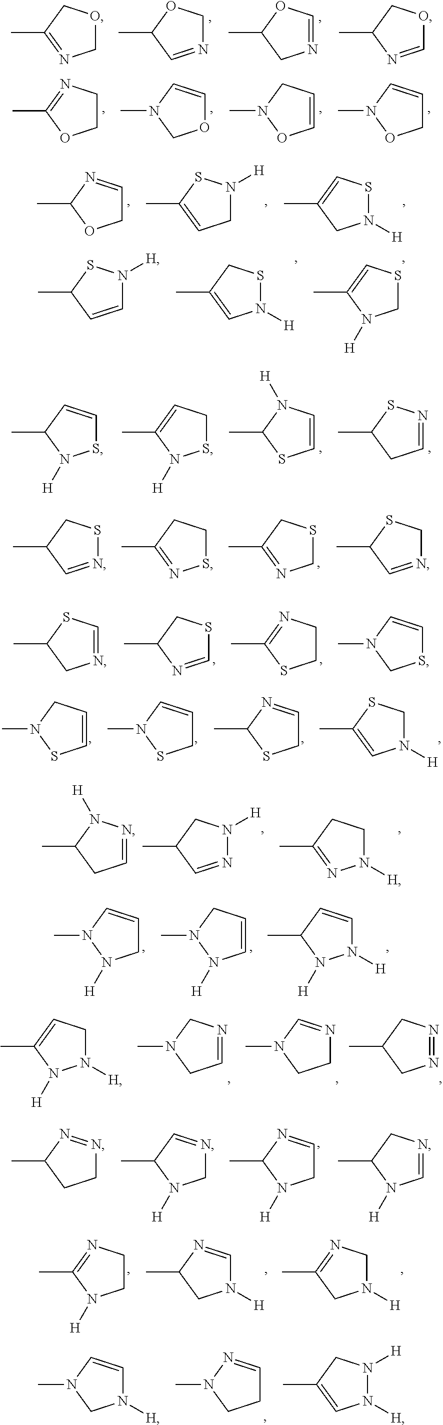

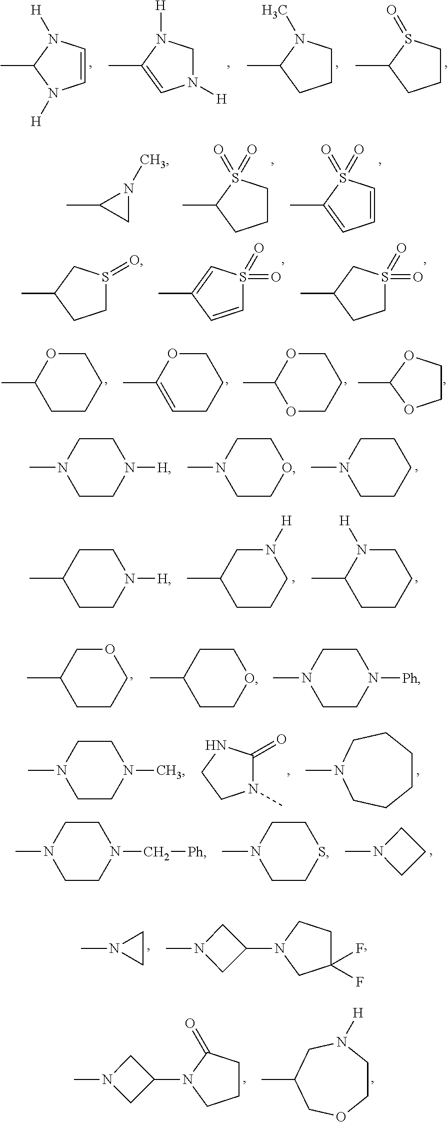

wherein these residues can be substituted with 1 to 5 substituents selected from R^{N1}, R^{N2}, Z¹ to Z¹². However it is clear to a skilled person that the term “can be substituted” refers to the replacement of a hydrogen atom by one of the substituents R^{N1}, R^{N2}, Z¹ to Z¹². Moreover it is clear to a skilled person that only these hydrogen atoms which are present in the residue can be replaced by the substituents R^{N1}, R^{N2}, Z¹ to Z¹². In case of secondary amine group in these heteroaryl residues, a hydrogen atom of secondary amine group is replaced by the substituent R^{N1} or R^{N2}. Thus, since the oxirane group (also named as ethylene oxide group) has only three hydrogen atoms, only three hydrogen atoms can be replaced by three substituents selected from Z¹ to Z¹². The carbon atom number of C₁-C₉ refers only to the carbon atoms of the heterocyclic ring system (heterocyclyl) and does not include the carbon atoms of the substituents R^{N1}, R^{N2}, Z¹ to Z¹².

As used herein, C₄-C₁₁ bicyclic carbocyclyl is represented by generalized formula (a1),

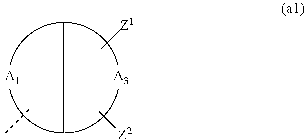

wherein, A₁, and A₃ represent independently C₁-C₇ alkylene; and C₅-C₁₁ bicyclic carbocyclic ring may be optionally substituted with 1 to 5 substituents selected from Z¹ to Z¹², preferably substituted by Z¹ and Z² and have 0-2 double bonds

Examples of preferred C₄-C₁₁ bicyclic carbocyclic ring are

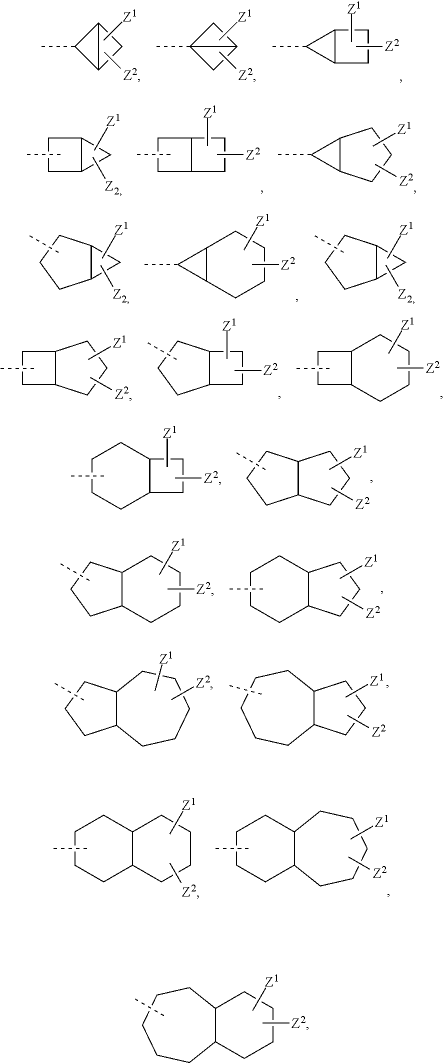

As used herein, C₁-C₁₀ bicyclic heterocyclyl may be represented by generalized formula (a2)

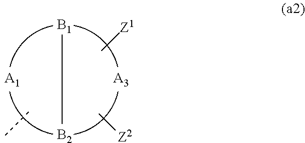

wherein, A₁, and A₃ represent independently C₁-C₅ alkylene and at least one carbon atom of said C₁-C₅ alkylene is replaced with heteroatoms selected from O, N, and S; and
B₁, and B₂ represent independently CH, or N;
C₅-C₁₁ bicyclic heterocyclic ring may be optionally substituted with 1 to 5 substituents selected from Z¹ to Z¹², R^{N1} and R^{N2}, preferably substituted by Z¹, Z² and R^{N1}, and have 0-3 double bonds.

Examples of preferred C₅-C₁₁ bicyclic heterocyclic ring are

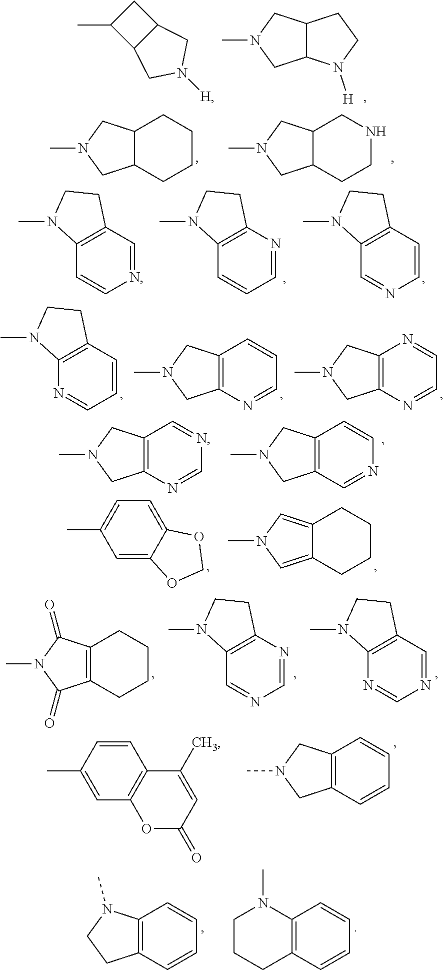

As used herein, C₄-C₁₁ bridged carbocyclyl may be represented by generalized formula (b1)

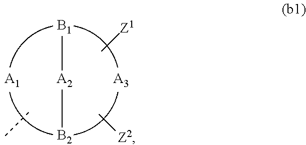

wherein A₁, A₂, and A₃ represent independently C₁-C₃ alkylene; and
B₁, B₂, represent independently CH, or B₁ and B₂ form a bond;
C₄-C₁₁ bridged carbocyclic ring may be optionally substituted with 1 to 5 substituents selected from Z¹ to Z¹², preferably substituted by Z¹ and Z² and have 0-2 double bonds.

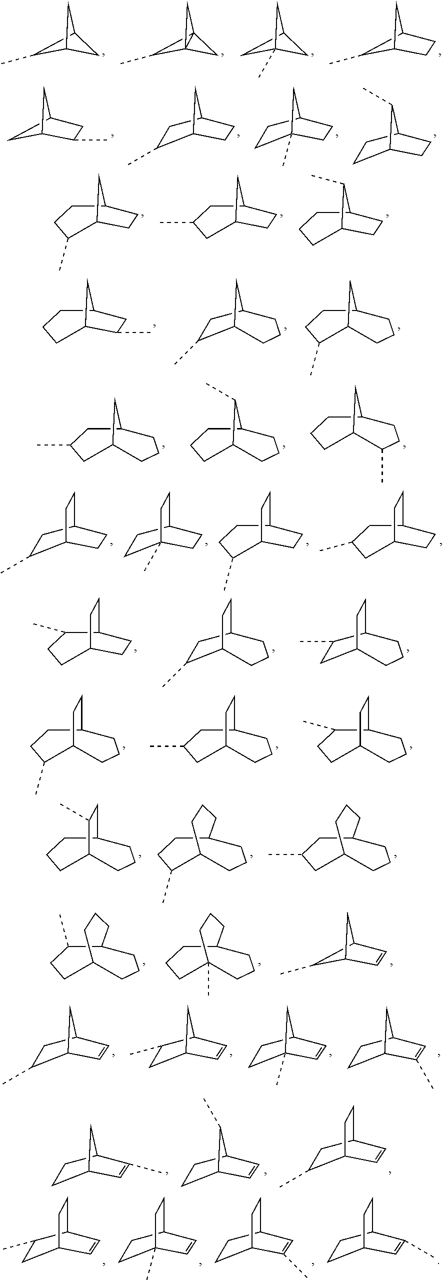

As used herein, C₁-C₁₀ bridged heterocyclyl may be represented by generalized formula (b2)

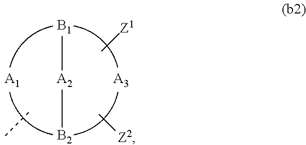

wherein, A₁, A₂, and A₃ represent independently C₁-C₃ alkylene and at least one carbon atom of said C₁-C₃ alkylene is replaced with heteroatoms selected from O, N, and S; and B₁, and B₂, represent independently CH, or N;
C₅-C₁₁ bridged heterocyclyl ring may be optionally substituted with 1 to 5 substituents selected from Z¹ to Z¹², R^{N1} and R^{N2}, preferably substituted by Z¹, Z² and R^{N1}, and have 0-2 double bonds.

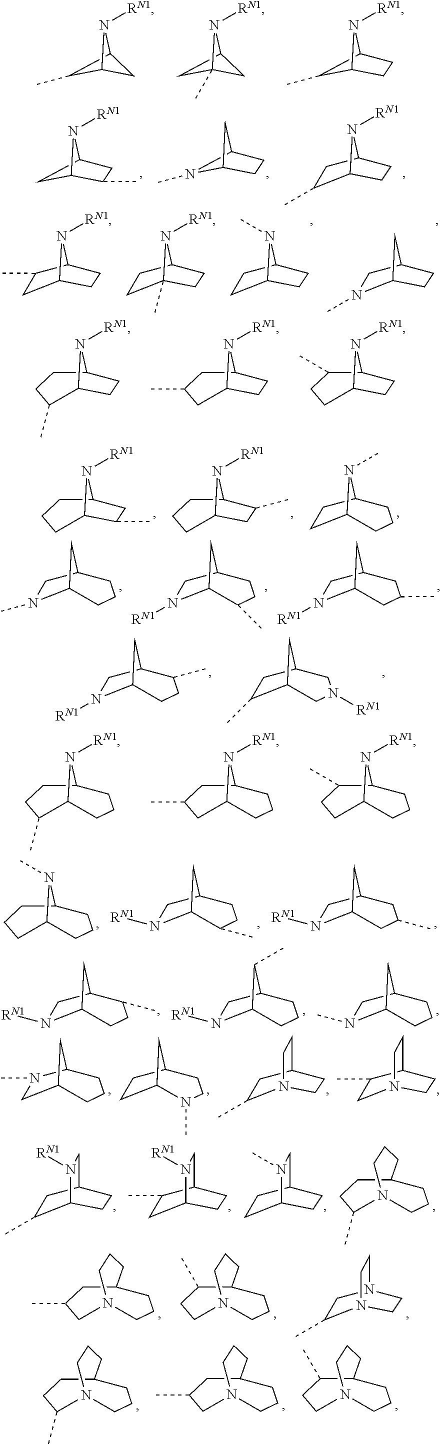

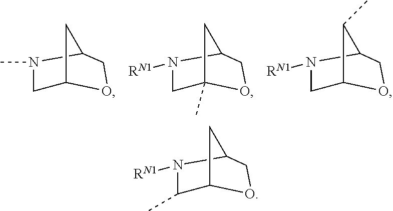

As used herein, C₇-C₁₆-spiroalkyl may be represented by generalized formula (W)

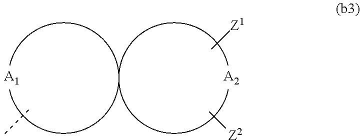

wherein A₁, and A₂, represent independently C₂-C₇ alkylene; and C₇-C₁₆ spiroaklyl may be optionally substituted with 1 to 5 substituents selected from Z¹ to Z¹², preferably substituted by Z¹-Z³, or by Z⁵-Z⁷ and have 0-2 double bonds.

As used herein, the term “C₇-C₁₆-spiroalkyl” refers to spirocarbocyclic residues, wherein these spirocarbocyclic residues can be substituted with 1 to 3 substituents selected from Z¹, Z², Z³, Z⁵, Z⁶ and Z⁷. However it is clear to a skilled person that the term “can be substituted” refers to the replacement of a hydrogen atom by one of the substituents Z¹, Z², Z³, Z⁵, Z⁶ and Z⁷. It is also possible that two of the substituents Z¹, Z², Z³, Z⁵, Z⁶ and Z⁷ represent together an oxygen atom and form together with the carbon atom of the spiroalkyl residue to which they are both attached a carbonyl moiety. The carbon atom number of C₇-C₁₆ refers only to the carbon atoms of the spiro ring system and does not include the carbon atoms of the substituents. Thus a spiro[4,5]decyl residue is counted as a C₁₀-spiroalkyl regardless if this spiro residue carries five pentyl substituents.

Examples of preferred C₇-C₁₆-spiroalkyl groups and substituted C₇-C₁₆-spiroalkyl groups are

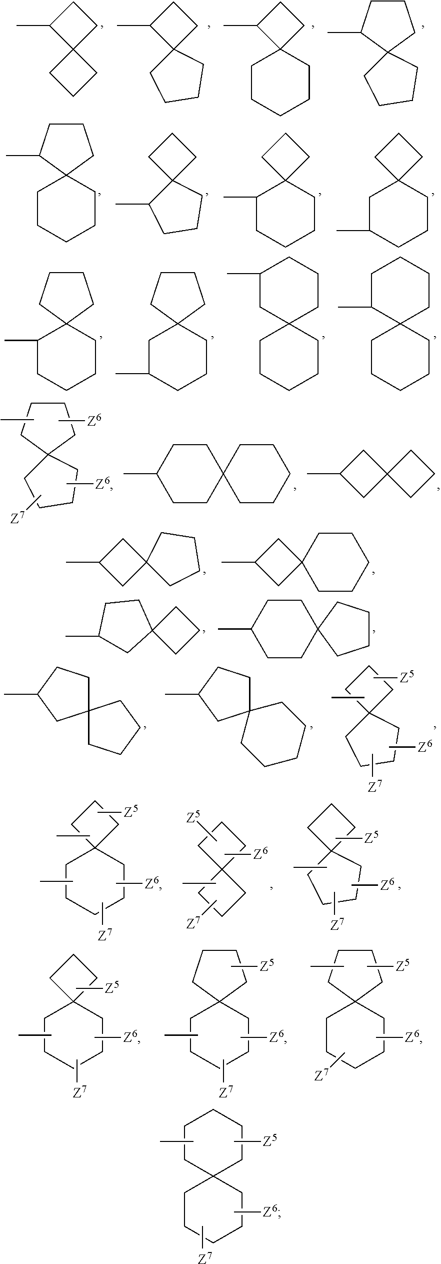

preferred substituents Z⁵, Z⁶ and Z⁷ are —H, —CH₃, —C₂H₅, —C₃H₇, —CH(CH₃)₂, —OH, —OCH₃, —OC₂H₅, —OC₃H₇, —NH₂, —N(CH₃)₂, —F, —Cl, —Br, —I, —ON, —CH₂F, —OHF₂, —OF₃, —OCHF₂, —OCF₃, more preferably, at least one of Z⁵, Z⁶ and Z⁷ is not —H.

of course, instead of Z⁵-Z⁷, Z¹-Z³ can be substituted and Z¹-Z³ have the same meanings of as defined in of Z⁵-Z⁷.

As used herein, the term “C₅-C₁₄-spiroheterocyclyl” may be represented by generalized formula (b4)

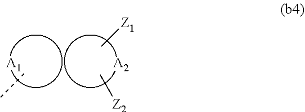

wherein A₁, and A₂, represent independently C₂-C₆ alkylene and at least one carbon atom of said C₂-C₆ alkylene is replaced with heteroatoms selected from O, N, and S; and C₅-C₁₄ spiroheterocyclyl ring may be optionally substituted with 1 to 5 substituents selected from Z¹ to Z¹², R^{N1} and R^{N2}, preferably substituted by R^{N1}, Z¹ and Z² and have 0-2 double bonds.

As used herein, the term “C₅-C₁₄-spiroheterocyclyl” refers to spiro residues with one, two or three heteroatoms such as O, S, N in the spiro ring system, wherein these spiroheterocyclic residues can be optionally substituted with 1 to 5 substituents selected from Z¹ to Z¹², R^{N1} and R^{N2}, preferably substituted with 1 to 3 substituents selected from R^{N1}, Z¹, Z², Z³, Z⁵, Z⁶ and Z⁷. However it is clear to a skilled person that the term “can be substituted” refers to the replacement of a hydrogen atom by one of the substituents R^{N1}, Z⁵, Z⁶ and Z⁷. The carbon atom number of C₅-C₁₄ refers only to the carbon atoms of the spiro ring system and does not include the carbon atoms of the substituents. Thus, a azaspiro[4,5]decyl residue is counted as a C₉-spiroalkyl regardless if this azaspiro[4,5]decyl residue carries five isopropyl substituents.

Examples of preferred C₅-C₁₄-spiroheterocyclyl groups and substituted C₅-C₁₄-spiroheterocyclyl groups are

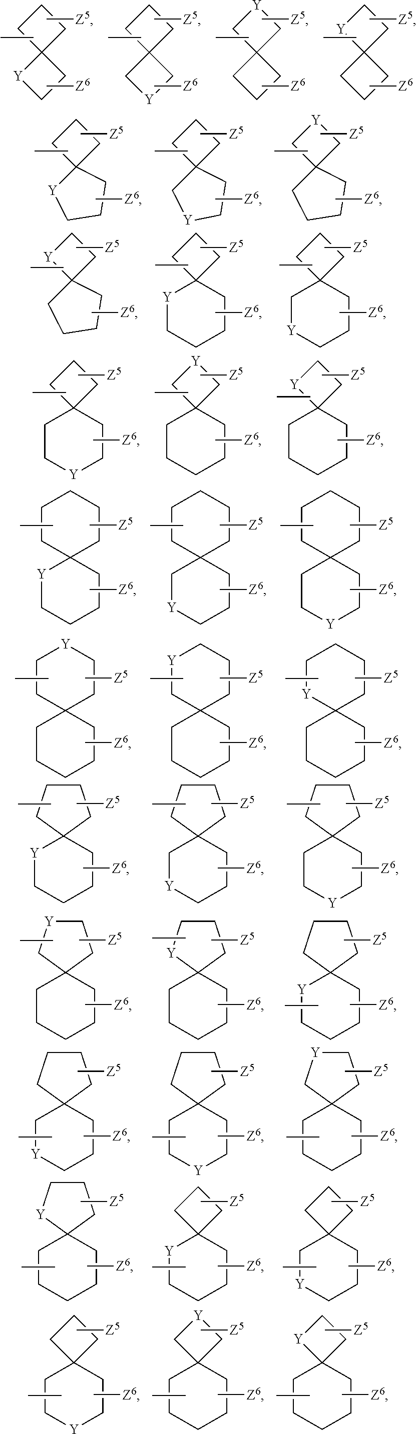

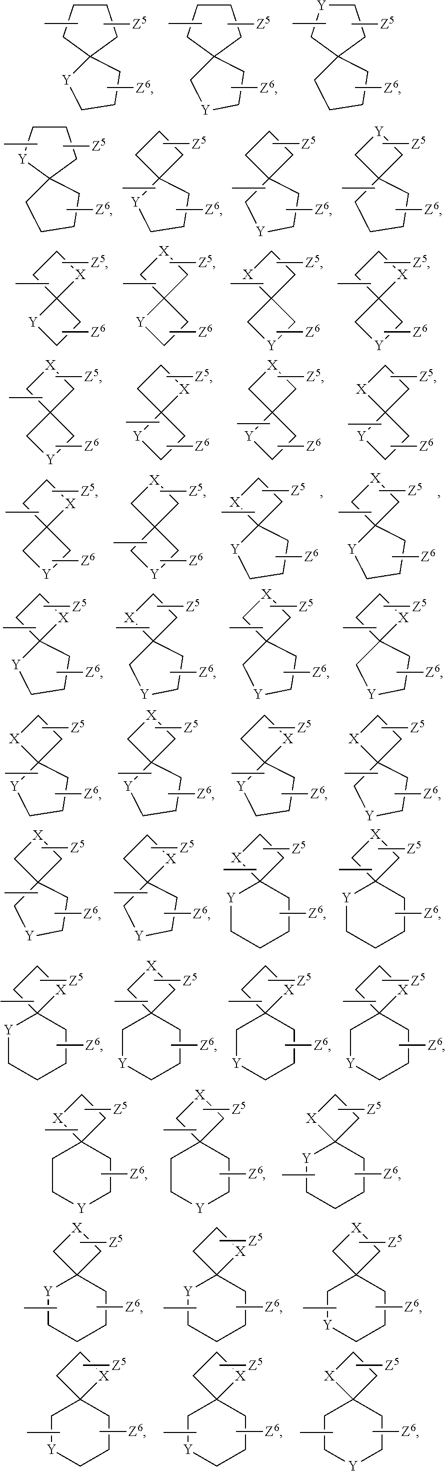

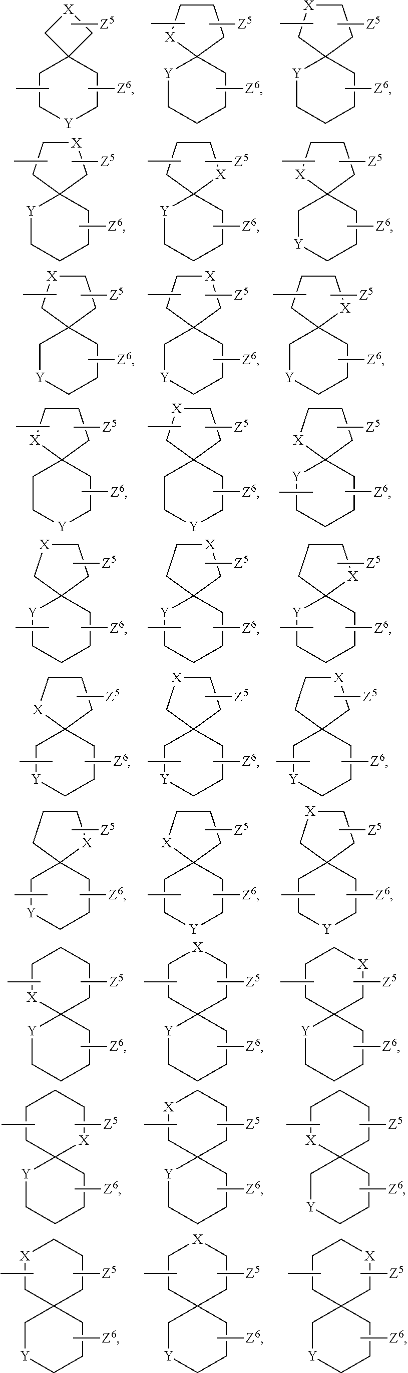

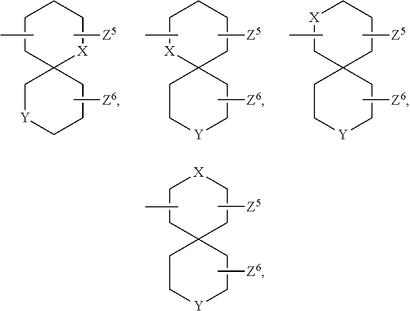

wherein Y and X represent independently of each other —O—, —NH—, —NR^{N1}—, —SO—, or —SO₂—, preferably —NH— or —NR^{N1}—, and Z⁵ and Z⁶ represent independently of each other —H, —CH₃, —C₂H₅, —C₃H₇, —CH(CH₃)₂, —OH, —OCH₃, —OC₂H₅, —OC₃H₇, —NH₂, —N(CH₃)₂, —F, —Cl, —Br, —I, —CN, —CH₂F, —CHF₂, —CF₃, —OCHF₂, or —OCF₃, more preferably, at least one of Z⁵, and Z⁶ is not —H.

Of course, instead of Z⁵ and Z⁶, Z¹ and Z² can be substituted and Z¹ and Z² have the same meanings of as defined in of Z⁵ and Z⁶.

Examples of preferred 4-membered heterocyclic groups and substituted 4-membered heterocyclic groups for R⁸, R⁹, and R¹⁴ are

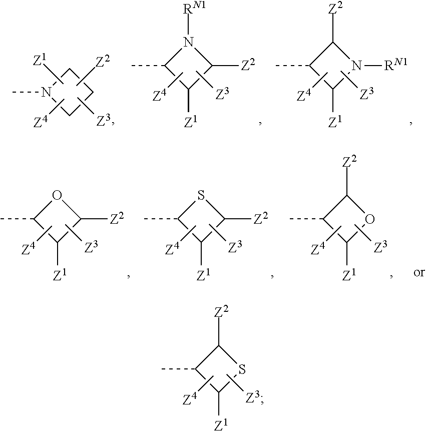

Preferably Z¹ to Z⁴ represent independently of each other —H, —CH₃, —C₂H₅, —C₃H₇, —CH(CH₃)₂, —OH, —OCH₃, —OC₂H₅, —OC₃H₇, —NH₂, —N(CH₃)₂, —F, —Cl, —Br, —I, —CN, —CH₂F, —CHF₂, —CF₃, —OCHF₂, or —OCF₃, more preferably, at least one of Z¹ to Z⁴ is not —H.

Preferred substituents for R^{N1} are —R¹⁵, —H, —CH₃, —C₂H₅, —C₃H₇, —CH(CH₃)₂, —CHF₂, —CF₃,

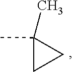

cyclo-C₃H₅, cyclo-C₄H₇, cyclo-C₅H₉, cyclo-C₆H₁₁, cyclo-C₇H₁₃, —C₄H₉, —CH₂—CH(CH₃)₂, —CH(CH₃)—C₂H₅, —C(CH₃)₃, —CH₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, —C₇H₁₅, —C₈H₁₇, -Ph, —CH₂-Ph, —CH₂—CH₂-Ph, —C₂H₄—CH═CH₂, —CH₂—CH═CH—CH₃, —CH₂—C(CH₃)=CH₂, —CH(CH₃)—CH═CH₂, —CH₂—CH═C(CH₃)₂, —CH₂—C≡CH, —C₂H₄—C≡CH, —CH₂—C≡C—CH₃, —COCH₃, —COC₂H₅, —COC₃H₇, —CO-cyclo-C₃H₅, —COCH(CH₃)₂, —COC(CH₃)₃, —COOH, —COOCH₃, —COOC₂H₅, —COOC₃H₇, —COO-cyclo-C₃H₅, —COOCH(CH₃)₂, —COOC(CH₃)₃, —COOCH₂Ph, —CONH₂, —CONHCH₃, —CONHC₂H₅, —CONHC₃H₇, —CONH-cyclo-C₃H₅, —CONH[CH(CH₃)₂], —CONH[C(CH₃)₃], —CON(CH₃)₂, —CON(C₂H₅)₂, —CON(C₃H₇)₂, —CON(cyclo-C₃H₅)₂, —CON[CH(CH₃)₂]₂, —CON[C(CH₃)₃]₂, or —SO₃H.

Preferred 4-membered heterocyclyl groups are

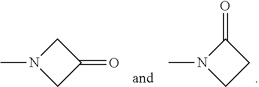

Examples of preferred 5-membered heterocyclyl groups and substituted 5-membered heterocyclyl groups for R⁸ or R⁹ are substituted or non substituted ring systems of five atoms including at least one heteroatom such as O, S, SO, SO₂, N, NO, wherein these 5-membered heterocyclic residues can be substituted with 1 to 4 substituents selected from R^{N1}, R^{N2}, Z¹, Z², Z³ and Z⁴. It is also possible that two of the substituents Z¹, Z², Z³ and Z⁴ represent together an oxygen atom and form together with the ring carbon atom of the heterocyclic ring to which they are both attached a carbonyl moiety or a sulfoxide moiety together with the ring sulphur atom to which they are attached or both Z substituents represent oxygen and form a sulfone moiety together with the ring sulphur atom to which they are attached. If the 5-membered heterocyclic residue contains a nitrogen atom which is substituted by R^{N1}. the first Z substituent represents R^{N1}. If the 5-membered heterocyclic residue contains two nitrogen atoms which are both substituted by one of the substituents R^{N1}, R^{N2}, the first Z substituent represents R^{N1}, and the second Z substituent represents R^{N2}. The same definition applies for the substituent R⁹ with the only difference that the optional substituents of the 5-membered heterocyclyl residue are Z⁵ to Z⁷ instead of Z¹ to Z⁴. Thus, for R⁹ the optional substituent Z¹ is replaced by Z⁵, Z² is replaced by Z⁶, Z³ is replaced by Z⁷, and Z⁴ is hydrogen.

Examples of preferred 5-membered heterocyclic groups and substituted 5-membered heterocyclic groups for R⁸ or R⁹ are

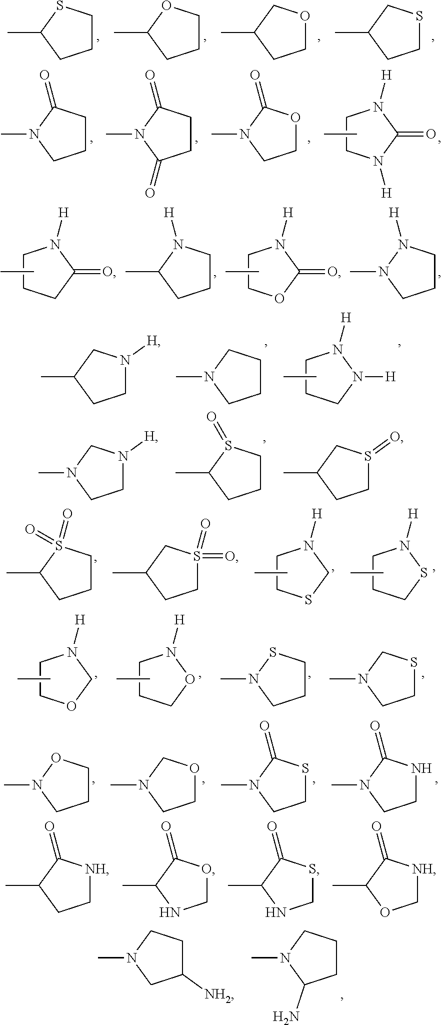

wherein the afore-mentioned 5-membered heterocyclic groups can be substituted with 1 to 4 substituents selected from R^{N1}, Z¹, Z², Z³ and Z⁴.

Examples of preferred 6-membered heterocyclic groups and substituted 6-membered heterocyclic groups for R⁸ or R⁹ are

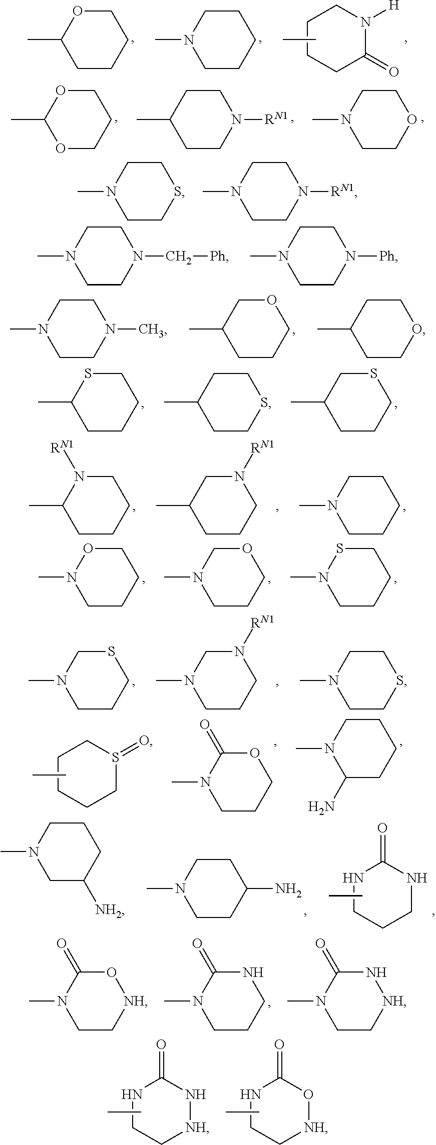

wherein the afore-mentioned 6-membered heterocyclic groups can be substituted with 1 to 4 substituents selected from Z¹, Z², Z³ and Z⁴. Preferred residues for the substituents Z¹ to Z⁴ are disclosed above.

Examples of preferred “monounsaturated 4-membered heterocyclyl” groups and substituted “monounsaturated 4-membered heterocyclyl” groups for R⁸ or R⁹ are substituted or non substituted ring systems of four atoms including at least one heteroatom such as O, S, SO, SO₂, N, NO, and one double bond, wherein these monounsaturated 4-membered heterocyclic residues can be substituted with 1 to 4 substituents selected from R^{N1}, R^{N2}, Z¹, Z², Z³ and Z⁴. However it is clear to a skilled person that the term “can be substituted” refers to the replacement of a hydrogen atom by one of the substituents R^{N1}, R^{N2}, Z¹, Z², Z³ or Z⁴. Moreover it is clear to a skilled person that only these hydrogen atoms which are present in the monounsaturated 4-membered heterocyclic residue can be replaced by the substituents R^{N1}, R^{N2}, Z¹, Z², Z³ and Z⁴. It is also possible that two of the substituents Z¹, Z², Z³ and Z⁴ represent together an oxygen atom and form together with the ring carbon atom of the heterocyclic ring to which they are both attached a carbonyl moiety or a sulfoxide moiety together with the ring sulphur atom to which they are attached or both Z substituents represent oxygen and form a sulfone moiety together with the ring sulphur atom to which they are attached. If the monounsaturated 4-membered heterocyclic residue contains a nitrogen atom, which is substituted by R^{N1}. the first substituent represents R^{N1}. If the monounsaturated 4-membered heterocyclic residue contains two nitrogen atoms which are both substituted by R^{N1}, R^{N2}, the first substituent represents R^{N1}, and the second substituent represents R^{N2}.

Examples of preferred monounsaturated 4-membered heterocyclic groups and substituted 4-membered heterocyclic groups for R⁸ or R⁹ are

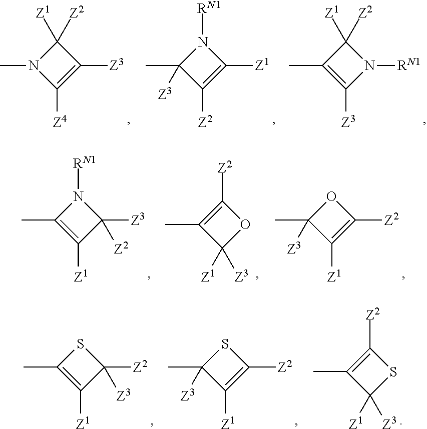

Preferably Z¹ to Z⁴ represent independently of each other —H, —CH₃, —C₂H₅, —C₃H₇, —CH(CH₃)₂, —OH, —OCH₃, —OC₂H₅, —OC₃H₇, —NH₂, —N(CH₃)₂, —F, —Cl, —Br, —I, —CN, —CH₂F, —CHF₂, —CF₃, —OCHF₂, or —OCF₃.

Preferred substituents for R^{N1} are —R¹⁵, —H, —CH₃, —C₂H₅, —C₃H₇, —CH(CH₃)₂, —CHF₂, —CF₃,

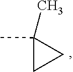

cyclo-C₃H₅, cyclo-C₄H₇, cyclo-C₅H₉, cyclo-C₆H₁₁, cyclo-C₇H₁₃, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, —C₇H₁₅, —C₈H₁₇, -Ph, —CH₂-Ph, —CH₂—CH₂-Ph, —C₂H₄—CH═CH₂, —CH₂—CH═CH—CH₃, —CH₂—C(CH₃)=CH₂, —CH(CH₃)—CH═CH₂, —CH₂—CH═C(CH₃)₂, —CH₂—C≡CH, —C₂H₄—C≡CH, —CH₂—C≡C—CH₃, —COCH₃, —COC₂H₅, —COC₃H₇, —CO-cyclo-C₃H₅, —COCH(CH₃)₂, —COC(CH₃)₃, —COOH, —COOCH₃, —COOC₂H₅, —COOC₃H₇, —COO-cyclo-C₃H₅, —COOCH(CH₃)₂, —COOC(CH₃)₃, —COOCH₂Ph, —CONH₂, —CONHCH₃, —CONHC₂H₅, —CONHC₃H₇, —CONH-cyclo-C₃H₅, —CONH[CH(CH₃)₂], —CONH[C(CH₃)₃], —CON(CH₃)₂, —CON(C₂H₅)₂, —CON(C₃H₇)₂, —CON(cyclo-C₃H₅)₂, —CON[CH(CH₃)₂]₂, —CON[C(CH₃)₃]₂, or —SO₃H.

Examples of preferred “monounsaturated 5-membered heterocyclyl” groups and substituted “monounsaturated 5-membered heterocyclyl” groups for R⁸ or R⁹ refers to substituted or non substituted ring systems of five atoms including at least one heteroatom such as O, S, SO, SO₂, N, NO, and one double bond, wherein these monounsaturated 5-membered heterocyclic residues can be substituted with 1 to 4 substituents selected from R^{N1}, R^{N2}, Z¹, Z², Z³ and Z⁴. It is also possible that two of the substituents R^{N1}, R^{N2}, Z¹, Z², Z³ and Z⁴ represent together an oxygen atom and form together with the ring carbon atom of the heterocyclic ring to which they are both attached a carbonyl moiety or a sulfoxide moiety together with the ring sulphur atom to which they are attached or both Z substituents represent oxygen and form a sulfone moiety together with the ring sulphur atom to which they are attached. If the monounsaturated 5-membered heterocyclic residue contains a nitrogen atom which is substituted by R^{N1}. the first substituent represents R^{N1}. If the monounsaturated 5-membered heterocyclic residue contains two nitrogen atoms which are both substituted by R^{N1}, R^{N2}, the first substituent represents R^{N1} and the second substituent represents R^{N2}.

Examples of preferred monounsaturated 5-membered heterocyclic groups and substituted 5-membered heterocyclic groups for R³ are

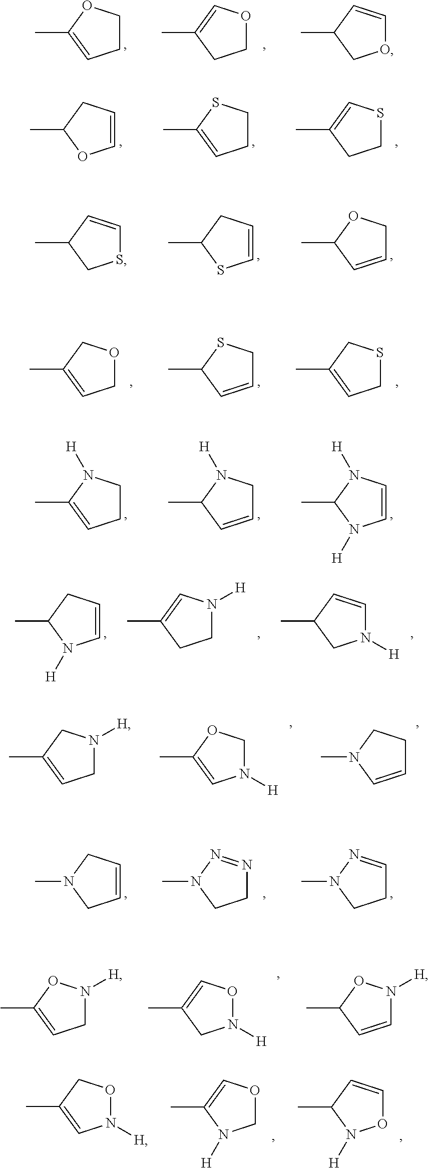

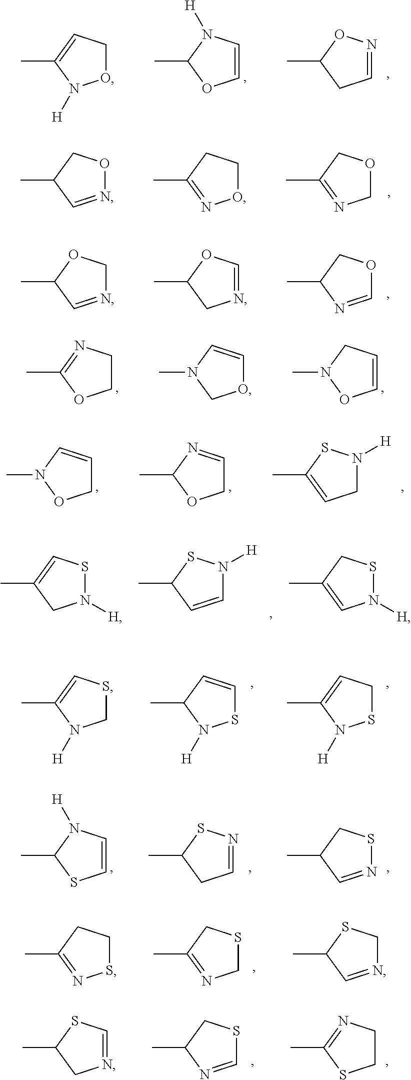

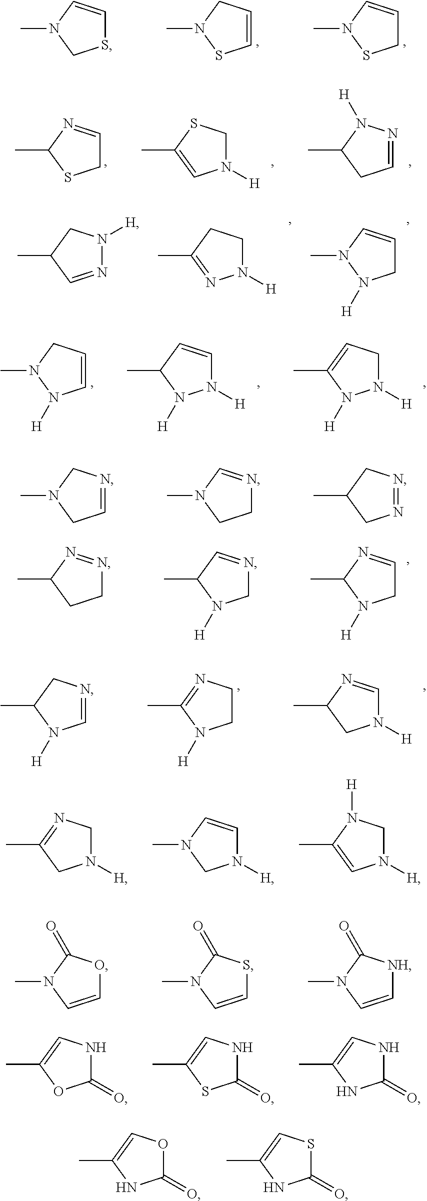

wherein the afore-mentioned monounsaturated 5-membered heterocyclic groups can be substituted with 1 to 4 substituents selected from R^{N1}, R^{N2}, Z¹, Z², Z³ and Z⁴.

R⁸, and R⁹ represents preferably independently of each other the following spiroheterocyclyl or C₅-C₁₄/N₀-N₂/O₀-O₂/S₀—S₁-spiroheterocyclyl residues: spiro[2,3]heterohexyl, spiro[2,4]heteroheptyl, spiro[2,5]heterooctyl, spiro[2,7]heterononyl, spiro[3,3]heteroheptyl, spiro[3,4]heterooctyl, spiro[3,5]heterononyl, spiro[3,6]heterodecyl, spiro[4,4]heterononyl, spiro[4,5]heterodecyl, spiro[4,6]heteroundecyl, spiro[5,5]heteroundecyl, spiro[5,6]heterododecyl, spiro[6,6]heterotridecyl, wherein the afore-mentioned spiroheterocyclyl or C₅-C₁₄/N₀-N₂/O₀-O₂/S₀—S₁-spiroheterocyclyl residues are linked through a ring carbon atom to the rest of the molecule and wherein the afore-mentioned spiroheterocyclyl or C₅-C₁₄/N₀-N₂/O₀-O₂/S₀—S₁-spiroheterocyclyl residues are optionally substituted with one to three substituents selected from R^{N1}, R^{N2}, Z¹, Z², Z³, Z⁵, Z⁶ and Z⁷. The heteroatom in the afore-mentioned spiroheterocyclyl or C₅-C₁₄/No-N₂/O₀-O₂/S₀—S₁-spiroheterocyclyl residues is preferably selected from —O—, —NH—, —NR^{N1}—, —NR^{N2}—, —SO—, and —SO₂—.

More preferably R⁸, and R⁹ represents independently of each other preferably the following spiroheterocyclyl or C₅-C₁₄/N₀-N₂/O₀-O₂/S₀—S₁-spiroheterocyclyl residues: azaspiro[3,3]heptyl, azaspiro[3,4]octyl, azaspiro[3,5]nonyl, azaspiro[3,6]decyl, azaspiro[4,4]nonyl, azaspiro[4,5]decyl, azaspiro[4,6]undecyl, azaspiro[5,5]undecyl, azaspiro[5,6]dodecyl, azaspiro[6,6]tridecyl, diazaspiro[3,3]heptyl, diazaspiro[3,4]octyl, diazaspiro[3,5]nonyl, diazaspiro[3,6]decyl, diazaspiro[4,4]nonyl, diazaspiro[4,5]decyl, diazaspiro[4,6]undecyl, diazaspiro[5,5]undecyl, diazaspiro[5,6]dodecyl, diazaspiro[6,6]tridecyl, triazaspiro[3,5]nonyl, triazaspiro[3,6]decyl, triazaspiro[4,5]decyl, triazaspiro[4,6]undecyl, triazaspiro[5,5]undecyl, triazaspiro[5,6]dodecyl, triazaspiro[6,6]tridecyl, oxazaspiro[3,3]heptyl, oxazaspiro[3,4]octyl, oxazaspiro[3,5]nonyl, oxazaspiro[3,6]decyl, oxazaspiro[4,4]nonyl, oxazaspiro[4,5]decyl, oxazaspiro[4,6]undecyl, oxazaspiro[5,5]undecyl, oxazaspiro[5,6]dodecyl, oxazaspiro[6,6]tridecyl, oxadiazaspiro[3,5]nonyl, oxadiazaspiro[3,6]decyl, oxadiazaspiro[4,5]decyl, oxadiazaspiro[4,6]undecyl, oxadiazaspiro[5,5]undecyl, oxadiazaspiro[5,6]dodecyl, oxadiazaspiro[6,6]tridecyl, wherein the afore-mentioned spiroheterocyclyl or C₅-C₁₄/N₀-N₂/O₀-O₂/S₀—S₁-spiroheterocyclyl residues are linked through a ring carbon atom to the rest of the molecule and wherein the afore-mentioned spiroheterocyclyl or C₅-C₁₄/N₀-N₂/O₀-O₂/S₀—S₁-spiroheterocyclyl residues are optionally substituted with one to three substituents selected from R^{N1}, R^{N2}, Z¹, Z², Z³, Z⁵, Z⁶ and Z⁷.

Also, R⁸ and R⁹ represents independently of each other more preferably the following residues: substituted or unsubstituted 4-membered carbocyclyl, substituted or unsubstituted 5-membered carbocyclyl, substituted or unsubstituted 6-membered carbocyclyl, 4-membered heterocyclyl, 5-membered heterocyclyl, 6-membered heterocyclyl, substituted 4-membered heterocyclyl, substituted 5-membered heterocyclyl, substituted 6-membered heterocyclyl, 4-membered nitrogenheterocyclyl, 5-membered nitrogenheterocyclyl, 6-membered nitrogenheterocyclyl, substituted 4-membered nitrogenheterocyclyl, substituted 5-membered nitrogenheterocyclyl, substituted 6-membered nitrogenheterocyclyl, spiro[2,3]heterohexyl, spiro[2,4]heteroheptyl, spiro[2,5]heterooctyl, spiro[2,7]heterononyl, spiro[3,3]heteroheptyl, spiro[3,4]heterooctyl, spiro[3,5]heterononyl, spiro[3,6]heterodecyl, spiro[4,4]heterononyl, spiro[4,5]heterodecyl, spiro[4,6]heteroundecyl, spiro[5,5]heteroundecyl, spiro[5,6]heterododecyl, spiro[6,6]heterotridecyl, substituted spiro[2,3]heterohexyl, substituted spiro[2,4]heteroheptyl, substituted spiro[2,5]heterooctyl, substituted spiro[2,7]heterononyl, substituted spiro[3,3]heteroheptyl, substituted spiro[3,4]heterooctyl, substituted spiro[3,5]heterononyl, substituted spiro[3,6]heterodecyl, substituted spiro[4,4]heterononyl, substituted spiro[4,5]heterodecyl, substituted spiro[4,6]heteroundecyl, substituted spiro[5,5]heteroundecyl, substituted spiro[5,6]heterododecyl, substituted spiro[6,6]heterotridecyl, azaspiro[3,3]heptyl, azaspiro[3,4]octyl, azaspiro[3,5]nonyl, azaspiro[3,6]decyl, azaspiro[4,4]nonyl, azaspiro[4,5]decyl, azaspiro[4,6]undecyl, azaspiro[5,5]undecyl, azaspiro[5,6]dodecyl, azaspiro[6,6]tridecyl, substituted azaspiro[3,3]heptyl, substituted azaspiro[3,4]octyl, substituted azaspiro[3,5]nonyl, substituted azaspiro[3,6]decyl, substituted azaspiro[4,4]nonyl, substituted azaspiro[4,5]decyl, substituted azaspiro[4,6]undecyl, substituted azaspiro[5,5]undecyl, substituted azaspiro[5,6]dodecyl, substituted azaspiro[6,6]tridecyl, diazaspiro[3,3]heptyl, diazaspiro[3,4]octyl, diazaspiro[3,5]nonyl, diazaspiro[3,6]decyl, diazaspiro[4,4]nonyl, diazaspiro[4,5]decyl, diazaspiro[4,6]undecyl, diazaspiro[5,5]undecyl, diazaspiro[5,6]dodecyl, diazaspiro[6,6]tridecyl, substituted diazaspiro[3,3]heptyl, substituted diazaspiro[3,4]octyl, substituted diazaspiro[3,5]nonyl, substituted diazaspiro[3,6]decyl, substituted diazaspiro[4,4]nonyl, substituted diazaspiro[4,5]decyl, substituted diazaspiro[4,6]undecyl, substituted diazaspiro[5,5]undecyl, substituted diazaspiro[5,6]dodecyl, substituted diazaspiro[6,6]tridecyl, triazaspiro[3,5]nonyl, triazaspiro[3,6]decyl, triazaspiro[4,5]decyl, triazaspiro[4,6]undecyl, triazaspiro[5,5]undecyl, triazaspiro[5,6]dodecyl, triazaspiro[6,6]tridecyl, substituted triazaspiro[3,5]nonyl, substituted triazaspiro[3,6]decyl, substituted triazaspiro[4,5]decyl, substituted triazaspiro[4,6]undecyl, substituted triazaspiro[5,5]undecyl, substituted triazaspiro[5,6]dodecyl, or substituted triazaspiro[6,6]tridecyl, oxazaspiro[3,3]heptyl, oxazaspiro[3,4]octyl, oxazaspiro[3,5]nonyl, oxazaspiro[3,6]decyl, oxazaspiro[4,4]nonyl, oxazaspiro[4,5]decyl, oxazaspiro[4,6]undecyl, oxazaspiro[5,5]undecyl, oxazaspiro[5,6]dodecyl, oxazaspiro[6,6]tridecyl, substituted oxazaspiro[3,3]heptyl, substituted oxazaspiro[3,4]octyl, substituted oxazaspiro[3,5]nonyl, substituted oxazaspiro[3,6]decyl, substituted oxazaspiro[4,4]nonyl, substituted oxazaspiro[4,5]decyl, substituted oxazaspiro[4,6]undecyl, substituted oxazaspiro[5,5]undecyl, substituted oxazaspiro[5,6]dodecyl, substituted oxazaspiro[6,6]tridecyl, oxadiazaspiro[3,5]nonyl, oxadiazaspiro[3,6]decyl, oxadiazaspiro[4,5]decyl, oxadiazaspiro[4,6]undecyl, oxadiazaspiro[5,5]undecyl, oxadiazaspiro[5,6]dodecyl, oxadiazaspiro[6,6]tridecyl, substituted oxadiazaspiro[3,5]nonyl, substituted oxadiazaspiro[3,6]decyl, substituted oxadiazaspiro[4,5]decyl, substituted oxadiazaspiro[4,6]undecyl, substituted oxadiazaspiro[5,5]undecyl, substituted oxadiazaspiro[5,6]dodecyl, or substituted oxadiazaspiro[6,6]tridecyl, wherein the afore-mentioned substituted or non-substituted spiroheterocyclyl or C₅-C₁₄/N₀-N₂/O₀-O₂/S₀—S₁-spiroheterocyclyl residues are linked through a ring carbon atom to the rest of the molecule and wherein the afore-mentioned substituted or non-substituted spiroheterocyclyl or C₅-C₁₄/N₀-N₂/O₀-O₂/S₀—S₁-spiroheterocyclyl residues are optionally substituted with one to three substituents selected from Z¹, Z², Z³, Z⁵, Z⁶ and Z⁷. The heteroatom in the afore-mentioned substituted or non-substituted spiroheterocyclyl or C₅-C₁₄/N₀-N₂/O₀-O₂/S₀—S₁-spiroheterocyclyl residues is preferably selected from —O—, —NH—, —NR^{N1}—, —SO—, and —SO₂—.

Preferably Z⁵, Z⁶ and Z⁷ represent independently of each other —H, —CH₃, —C₂H₅, —C₃H₇, —CH(CH₃)₂, —OH, —OCH₃, —OC₂H₅, —OC₃H₇, —NH₂, —N(CH₃)₂, —F, —Cl, —Br, —I, —CN, —CH₂F, —CHF₂, —CF₃, —OCHF₂, or —OCF₃.

If present, R^{N1} is preferably selected from: —R¹⁵, —H, —CH₃, —C₂H₅, —C₃H₇, —CH(CH₃)₂, —CHF₂, —CF₃,

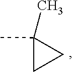

cyclo-C₃H₅, cyclo-C₄H₇, cyclo-C₅H, cyclo-C₆H₁₁, cyclo-C₇H₁₃, —C₄H, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, —C₇H₁₅, —C₈H₁₇, -Ph, —CH₂-Ph, —CH₂—CH₂-Ph, —C₂H₄—CH═CH₂, —CH₂—CH═CH—CH₃, —CH₂—C(CH₃)=CH₂, —CH(CH₃)—CH═CH₂, —CH₂—CH═C(CH₃)₂, —CH₂—C≡CH, —C₂H₄—C≡CH, —CH₂—C≡C—CH₃, —COCH₃, —COC₂H₅, —COC₃H₇, —CO-cyclo-C₃H₅, —COCH(CH₃)₂, —COC(CH₃)₃, —COOH, —COOCH₃, —COOC₂H₅, —COOC₃H₇, —COO-cyclo-C₃H₅, —COOCH(CH₃)₂, —COOC(CH₃)₃, —COOCH₂Ph, —CONH₂, —CONHCH₃, —CONHC₂H₅, —CONHC₃H₇, —CONH-cyclo-C₃H₅, —CONH[CH(CH₃)₂], —CONH[C(CH₃)₃], —CON(CH₃)₂, —CON(C₂H₅)₂, —CON(C₃H₇)₂, —CON(cyclo-C₃H₅)₂, —CON[CH(CH₃)₂]₂, —CON[C(CH₃)₃]₂, or —SO₃H.

The term “4-membered nitrogenheterocyclyl” refers to the residue “4-membered heterocyclyl” as defined above, wherein at least one heteroatom is a nitrogen atom and the residue is linked through the at least one nitrogen ring atom to the rest of the molecule and wherein Z¹ is replaced by Z⁵, Z² is replaced by Z⁶, Z³ is replaced by Z⁷, and Z⁴ is hydrogen.

The term “5-membered nitrogenheterocyclyl” refers to the residue “5-membered heterocyclyl” as defined above, wherein at least one heteroatom is a nitrogen atom and the residue is linked through the at least one nitrogen ring atom to the rest of the molecule and wherein Z¹ is replaced by Z⁵, Z² is replaced by Z⁶, Z³ is replaced by Z⁷, and Z⁴ is hydrogen.

The term “6-membered nitrogenheterocyclyl” refers to the residue “6-membered heterocyclyl” as defined above, wherein at least one heteroatom is a nitrogen atom and the residue is linked through the at least one nitrogen ring atom to the rest of the molecule and wherein Z¹ is replaced by Z⁵, Z² is replaced by Z⁶, Z³ is replaced by Z⁷, and Z⁴ is hydrogen.

Still more preferably R⁸ or R⁹ is selected from the following residues:

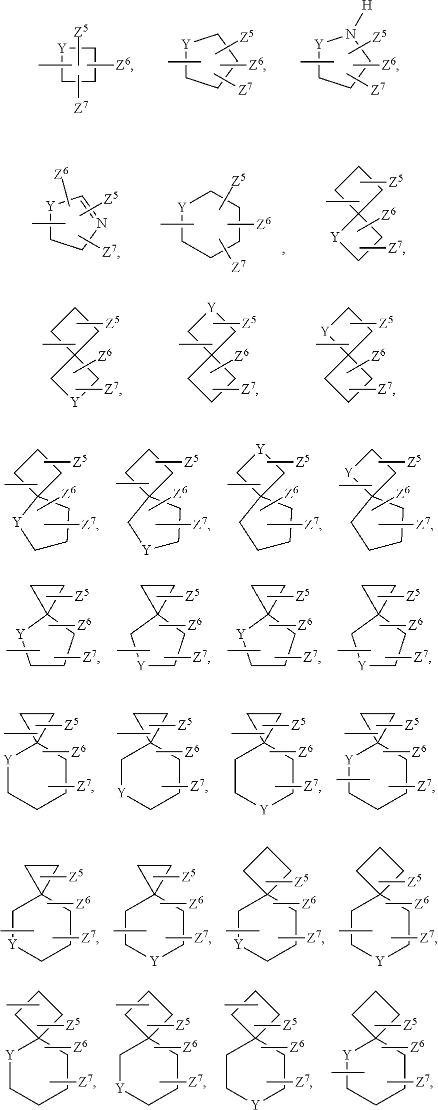

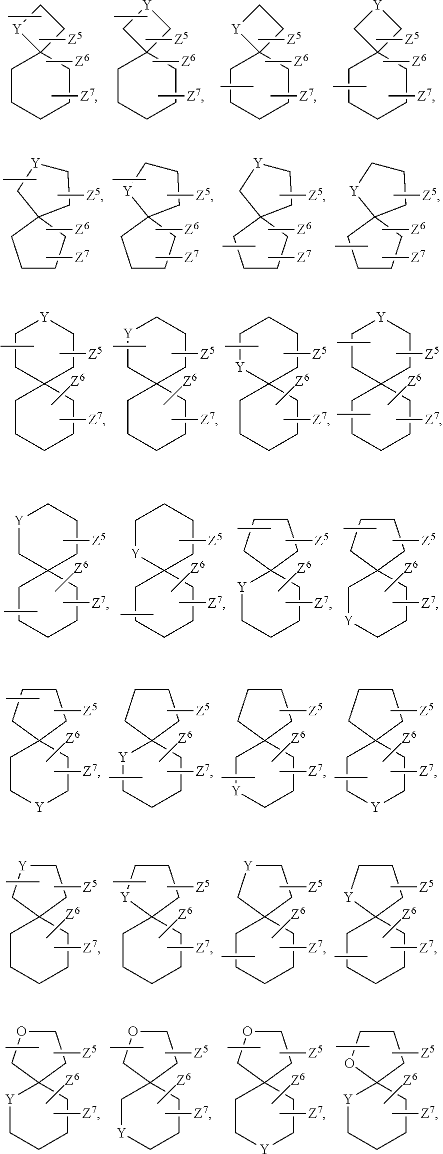

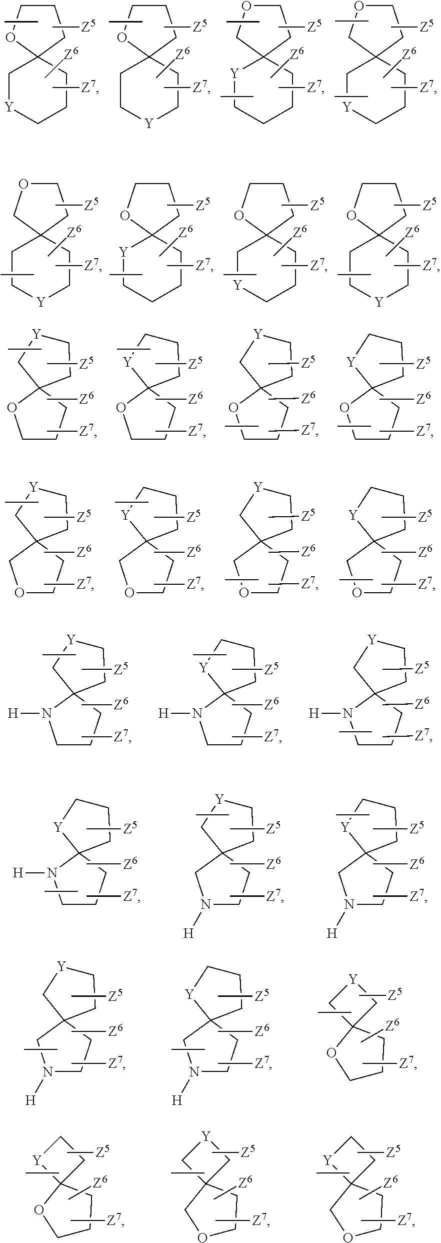

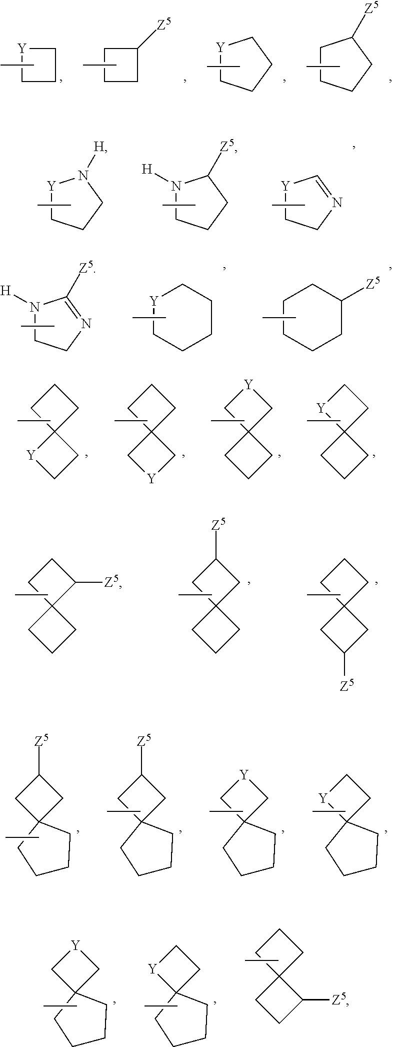

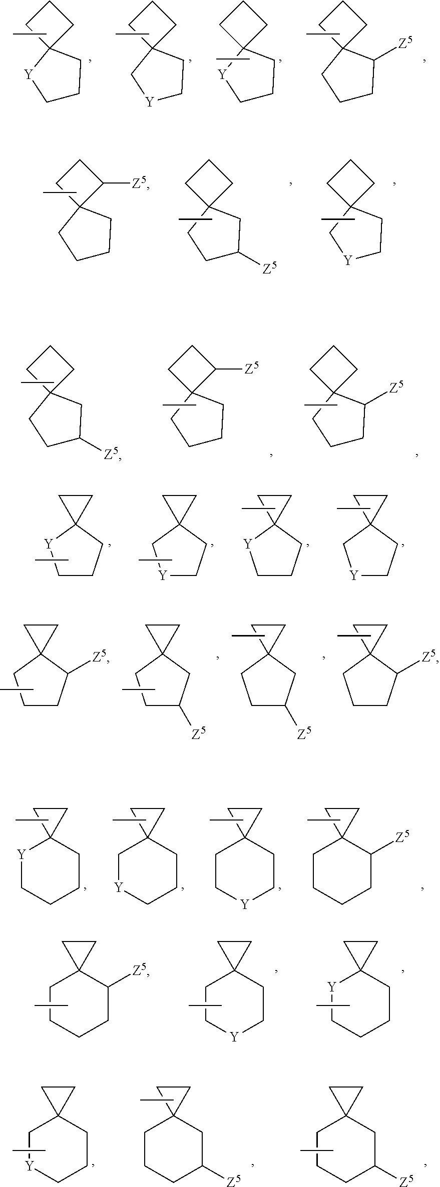

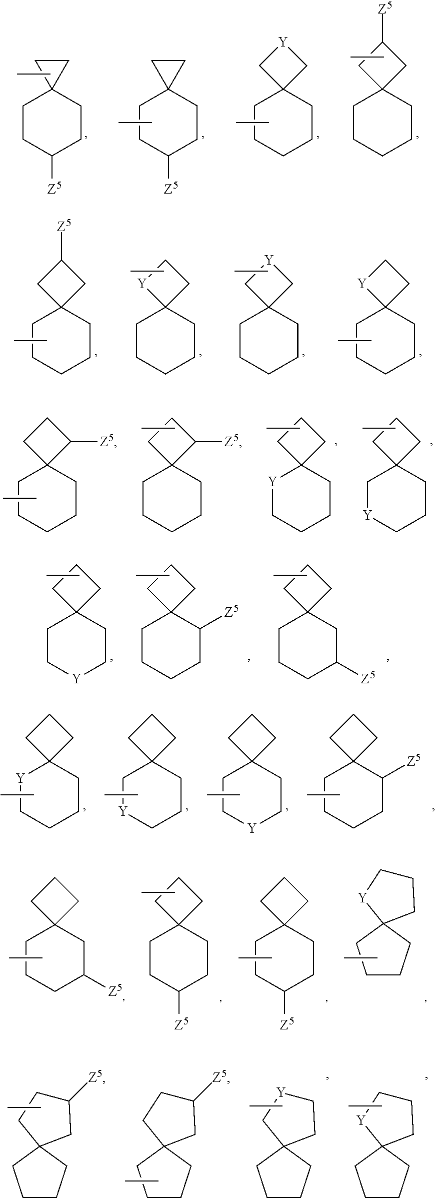

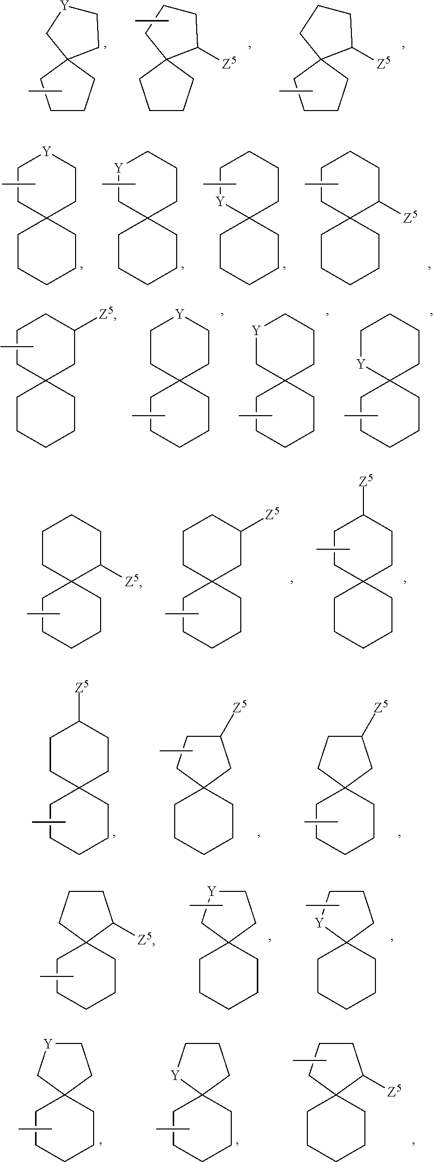

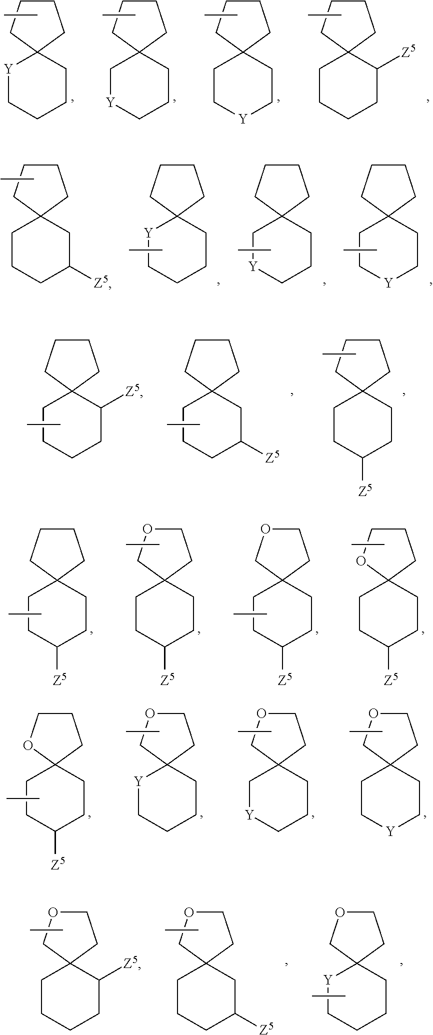

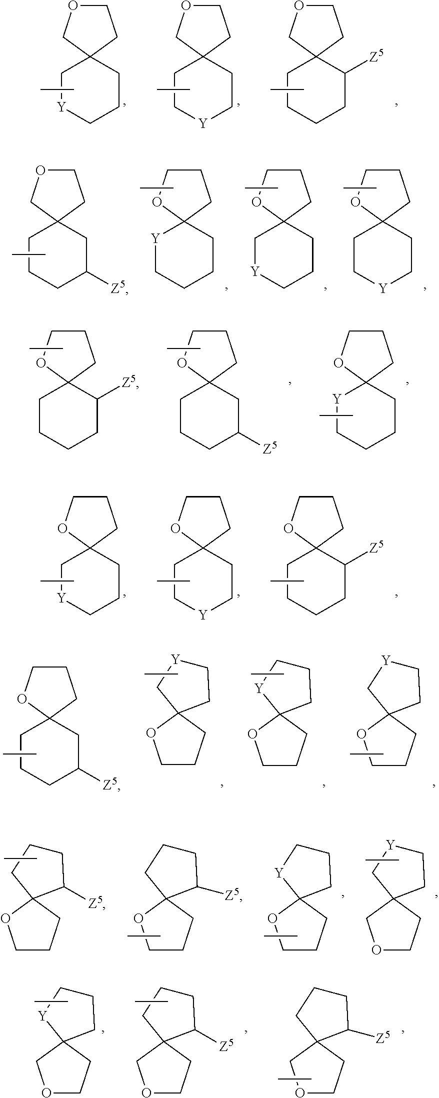

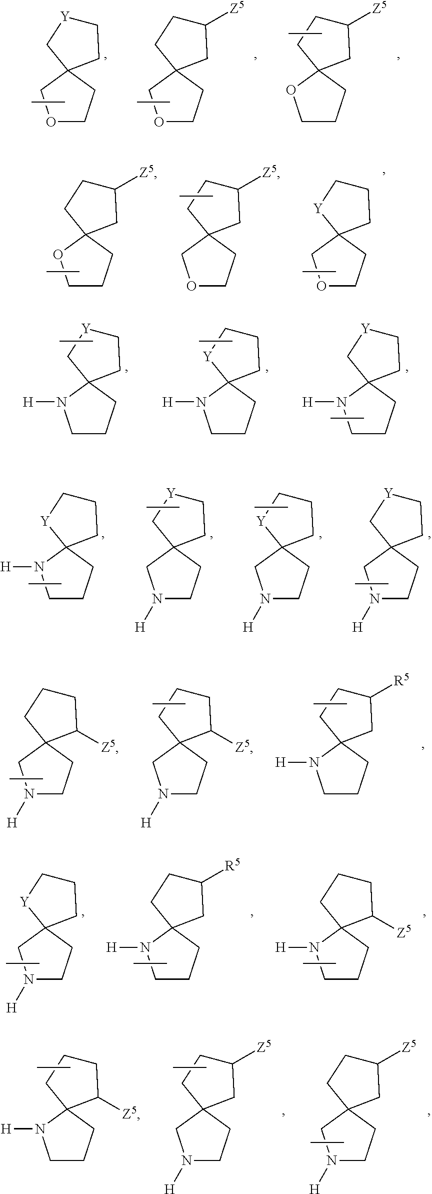

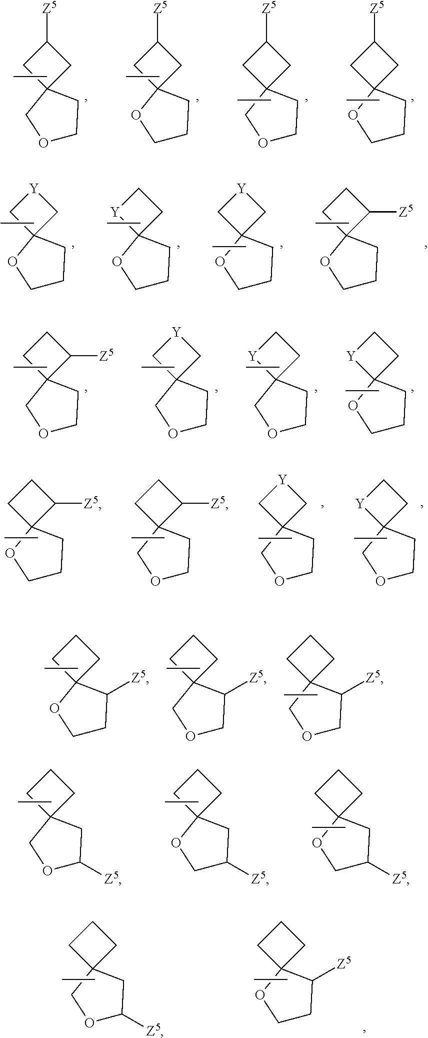

wherein Y represents —O—, —NH—, —NR^{N1}—, —NR^{N2}—, —SO—, or —SO₂—, preferably —NH— or —NR^{N1}— and wherein the substituents Z⁵, Z⁶ and Z⁷ have the meanings as defined herein. Of course, substituents Z⁵, Z⁶ and Z⁷ can be replaced with the substitutents Z¹, Z², Z³, and Z¹, Z², Z³, have the meanings as defined herein.

As used herein, the term “spironitrogencyclyl” refers to the C₅-C₁₄/N₁—N₃-spironitrogencyclyl residues comprising or including the C₅-C₁₄-spiroheterocyclyl residues as disclosed above, wherein the heteroatom is nitrogen, i.e. Y is NH, NR^{N1} or NR^{N2}. The term “C₅-C₁₄/N₁—N₃” means that the spiro ring system consists of 5 to 14 carbon atoms and 1 to 3 nitrogen atoms. Moreover the spironitrogencyclyl residues can be substituted with 1 to 3 substituents selected from R^{N1}, R^{N2}, Z¹, Z², Z³, Z⁵, Z⁶ and Z⁷. However it is clear to a skilled person that the term “can be substituted” refers to the replacement of a hydrogen atom by one of the substituents R^{N1}, R^{N2}, Z¹, Z², Z³, Z⁵, Z⁶ or Z⁷. It is also possible that two of the substituents Z⁵, Z⁶ and Z⁷ represent together an oxygen atom and form together with the carbon atom of the spironitrogencyclyl residue to which they are both attached a carbonyl moiety. Moreover the C₅-C₁₄/N₁—N₃-spironitrogencyclyl residues are characterized in that the spironitrogencyclyl residue is linked through a nitrogen atom of the spiro ring system and not through a carbon atom of the spiro ring system. This means in regard to the above-mentioned C₅-C₁₄-spiroheterocyclyl residue that the heteroatom Y is nitrogen and that this C₅-C₁₄-spiroheterocycly residue is linked to the rest of the molecule through this nitrogen atom (which is Y). If the C₅-C₁₄/N₁—N₃-spironitrogencyclyl residue contains a second nitrogen atom, it is substituted by one of the substituents R^{N1}, R^{N2}. Thus the indication “N₂” refers to a first nitro gen atom through which the spironitrogencyclyl residue is linked and to the group

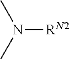

of the spiro ring system. If the spironitrogencyclyl residue contains a third nitrogen atom and both nitrogen atoms are substituted by one of the substituents Z¹, Z², Z³, Z⁵, Z⁶ and Z⁷, the first Z substituent on the second nitrogen atom represents R^{N2} and the second Z substituent on the third nitrogen atom represents R^{N1}. Thus, the indication “N₃” refers to a first nitrogen atom through which the spironitrogencyclyl residue is linked and to the groups

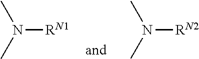

of the spiro ring system. Thus the C₅-C₁₄/N₁—N₃-spironitrogencyclyl residue can contain one, two or three nitrogen atoms in the spiro ring system. The numbers of atoms “C₅-C₁₄/N₁—N₂” do not include C and N atoms from the substituents Z¹, Z², Z³, Z⁵ to Z⁷.

As used herein, the term “nitrogenheterocyclyl” refers to C₅-C₁₄/N₁-N₃/O₀-O₂/S₀—S₁-nitrogenheterocyclyl residues comprising or including the C₅-C₁₄-spiroheterocyclyl residues as disclosed above, wherein the heteroatom is nitrogen, i.e. Y is NH, NR^{N1} or NR^{N2}. The term “C₅-C₁₄/N₁-N₃/O₀-O₂/S₀—S₁” means that the spiro ring system consists of 5 to 14 carbon atoms and 1 to 3 nitrogen atoms, 0 to 2 oxygen atoms and 0 or 1 sulfur atom. Moreover the nitrogenheterocyclyl residues can be substituted with 1 to 3 substituents selected from R^{N1}, R^{N2}, Z¹ to Z³, Z⁵, Z⁶ and Z⁷. However it is clear to a skilled person that the term “can be substituted” refers to the replacement of a hydrogen atom by one of the substituents Z¹, Z², Z³, Z⁵, Z⁶ or Z⁷. It is also possible that two of the substituents Z⁵, Z⁶ and Z⁷ represent together an oxygen atom and form together with the carbon atom of the nitrogenheterocyclyl residue to which they are both attached a carbonyl moiety. Moreover the C₅-C₁₄/N₁-N₃/O₀-O₂/S₀—S₁-nitrogenheterocyclyl residues are characterized by that the nitrogenheterocyclyl residue is linked through a nitrogen atom of the spiro ring system and not through a carbon atom of the spiro ring system. This means in regard to the above-mentioned C₅-C₁₄-spiroheterocycly residue that the heteroatom Y is nitrogen and that this C₅-C₁₄-spiroheterocycly residue is linked to the rest of the molecule through this nitrogen atom (which is Y). If the C₅-C₁₄/N₁-N₃/O₀-O₂/S₀—S₁-nitrogenheterocyclyl residue contains a second nitrogen atom which is substituted by one of the substituents Z¹, Z², Z³, Z⁵, Z⁶ and Z⁷, said Z substituent represents R^{N2}. Thus, the indication “N₂” refers to a first nitrogen atom through which the nitrogenheterocyclyl residue is linked and to the group

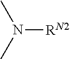

of the spiro ring system. If the nitrogenheterocyclyl residue contains a third nitrogen atom and both nitrogen atoms are substituted by one of the substituents Z⁵, Z⁶ and Z⁷, the first Z substituent on the second nitrogen atom represents R^{N2} and the second Z substituent on the third nitrogen atom represents R^{N1}. Thus, the indication “N₃” refers to a first nitrogen atom through which the nitrogenheterocyclyl residue is linked and to the groups

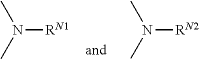

of the spiro ring system. The indication “S₁” refers to the group —S— or —SO— or —SO₂— of the spiro ring system. The indication “S₀” means that no sulfur is present in the nitrogenheterocyclyl residue. The indication “O₁” refers to the group —O— and the indication “O₂” to two groups —O— which are not directly linked to each other, while “O₀” indicates that no oxygen is present in the spiro ring system. Thus, the C₅-C₁₄/N₁-N₃/O₀-O₂/S₀—S₁-nitrogenheterocyclyl residue can contain in total 6 hetero atoms while in total not more than 3 hetero atoms should be present in the spiro ring system. Moreover it is preferred that the heteroatoms in the spiro ring system are not directly bound to each other. The numbers of atoms “C₅-C₁₄/N₁-N₃/O₀-O₂/S₀—S₁” do not include C, O, S and N atoms from the substituents R^{N1}, R^{N2}, Z¹ to Z³, Z⁵ to Z⁷.

Preferred is the presence of one nitrogen atom or two nitrogen atoms or one nitrogen atom and one sulfur atom or one nitrogen atom and one sulfoxide moiety or one nitrogen atom and one sulphone moiety or one nitrogen atom and one oxygen atom or one nitrogen atom and two oxygen atoms or one oxygen atom and two nitrogen atoms in the spiro ring system.

R⁸ and R⁹ represents independently of each other preferably the following spironitrogencyclyl, nitrogenheterocyclyl, C₅-C₁₄/N₁—N₃-spironitrogencyclyl or C₅-C₁₄/N₁-N₃/O₀-O₂/S₀—S₁-nitrogenheterocyclyl residues: 4-membered nitrogenheterocyclyl, 5-membered nitrogenheterocyclyl, 6-membered nitrogenheterocyclyl, 5-membered dinitrogenheterocyclyl, 6-membered dinitrogenheterocyclyl, spiro[2,3]heterohexyl, spiro[2,4]heteroheptyl, spiro[2,5]heterooctyl, spiro[2,7]heterononyl, spiro[3,3]heteroheptyl, spiro[3,4]heterooctyl, spiro[3,5]heterononyl, spiro[3,6]heterodecyl, spiro[4,4]heterononyl, spiro[4,5]heterodecyl, spiro[4,6]heteroundecyl, spiro[5,5]heteroundecyl, spiro[5,6]heterododecyl, spiro[6,6]heterotridecyl, wherein the afore-mentioned spironitrogencyclyl, nitrogenheterocyclyl, C₅-C₁₄/N₁—N₃-spironitrogencyclyl or C₅-C₁₄/N₁-N₃/O₀-O₂/S₀—S₁-nitrogenheterocyclyl residues are linked through a ring nitrogen atom to the rest of the molecule and wherein the afore-mentioned spironitrogencyclyl, nitrogenheterocyclyl, C₅-C₁₄/N₁—N₃-spironitrogencyclyl or C₅-C₁₄/N₁-N₃/O₀-O₂/S₀—S₁-nitrogenheterocyclyl residues are optionally substituted with one to three substituents selected from R^{N1}, R^{N2}, Z¹, Z², Z³, Z⁵, Z⁶ and Z⁷.

The term “5-membered dinitrogenheterocyclyl” refers to the residue “5-membered heterocyclyl” as defined above, wherein two heteroatoms are nitrogen atoms and the residue is linked through a nitrogen ring atom to the rest of the molecule and wherein Z¹ is replaced by Z⁵, Z² is replaced by Z⁶, Z³ is replaced by Z⁷, and Z⁴ is hydrogen.

The term “6-membered dinitrogenheterocyclyl” refers to the residue “6-membered heterocyclyl” as defined above, wherein two heteroatoms are nitrogen atoms and the residue is linked through a nitrogen ring atom to the rest of the molecule and wherein Z¹ is replaced by Z⁵, Z² is replaced by Z⁶, Z³ is replaced by Z⁷, and Z⁴ is hydrogen.

Moreover the afore-mentioned spironitrogencyclyl, nitrogenheterocyclyl, C₅-C₁₄/N₁—N₃-spironitrogencyclyl or C₅-C₁₄/N₁-N₃/O₀-O₂/S₀—S₁-nitrogenheterocyclyl residues contain at least one nitrogen atom through which these residues are linked to the rest of the molecule and may contain one or two further moieties selected from oxygen (—O—), sulfoxide (—SO—), sulfone (—SO₂—), carbonyl (—CO—) and nitrogen (—NR^{N1}—).

Preferable substituents Z⁵, Z⁶ and Z⁷ represent independently of each other —H, —CH₃, —C₂H₅, —C₃H₇, —CH(CH₃)₂, —OH, —OCH₃, —OC₂H₅, —OC₃H₇, —NH₂, —NHCH₃, —N(CH₃)₂, —F, —Cl, —Br, —I, —CN, —CH₂F, —CHF₂, —CF₃, —OCHF₂, or —OCF₃.

R^{N1}, and R^{N2} are preferably selected independently of each other from: —R¹⁵, —H, —CH₃, —C₂H₅, —C₃H₇, —CH(CH₃)₂, —CHF₂, —CF₃,

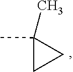

cyclo-C₃H₅, cyclo-C₄H₇, cyclo-C₅H₉, cyclo-C₆H₁₁, cyclo-C₇H₁₃, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, —C₇H₁₅, —C₈H₁₇, -Ph, —CH₂-Ph, —CH₂—CH₂-Ph, —C₂H₄—CH═CH₂, —CH₂—CH═CH—CH₃, —CH₂—C(CH₃)=CH₂, —CH(CH₃)—CH═CH₂, —CH₂—CH═C(CH₃)₂, —CH₂—C≡CH, —C₂H₄—C≡CH, —CH₂—C≡C—CH₃, —COCH₃, —COC₂H₅, —COC₃H₇, —CO-cyclo-C₃H₅, —COCH(CH₃)₂, —COC(CH₃)₃, —COOH, —COOCH₃, —COOC₂H₅, —COOC₃H₇, —COO-cyclo-C₃H₅, —COOCH(CH₃)₂, —COOC(CH₃)₃, —COOCH₂Ph, —CONH₂, —CONHCH₃, —CONHC₂H₅, —CONHC₃H₇, —CONH-cyclo-C₃H₅, —CONH[CH(CH₃)₂], —CONH[C(CH₃)₃], —CON(CH₃)₂, —CON(C₂H₅)₂, —CON(C₃H₇)₂, —CON(cyclo-C₃H₅)₂, —CON[CH(CH₃)₂]₂, —CON[C(CH₃)₃]₂, and —SO₃H.

More preferably R⁸ and R⁹ represents independently of each other the following spironitrogencyclyl, nitrogenheterocyclyl, C₅-C₁₄/N₁—N₃-spironitrogencyclyl or C₅-C₁₄/N₁-N₃/O₀-O₂/S₀—S₁-nitrogenheterocyclyl residues: 4-membered nitrogenheterocyclyl linked through the nitrogen atom to the rest of the molecule, 5-membered nitrogenheterocyclyl linked through the nitrogen atom to the rest of the molecule, 6-membered nitrogenheterocyclyl linked through the nitrogen atom, substituted 4-membered nitrogenheterocyclyl linked through the nitrogen atom, substituted 5-membered nitrogenheterocyclyl linked through the nitrogen atom, substituted 6-membered nitrogenheterocyclyl linked through the nitrogen atom, 5-membered dinitrogenheterocyclyl linked through a nitrogen atom, 6-membered dinitrogenheterocyclyl linked through a nitrogen atom, substituted 5-membered dinitrogenheterocyclyl linked through a nitrogen atom, substituted 6-membered dinitrogenheterocyclyl linked through a nitrogen atom to the rest of the molecule, wherein the afore-mentioned spironitrogencyclyl, nitrogenheterocyclyl, C₅-C₁₄/N₁—N₃-spironitrogencyclyl or C₅-C₁₄/N₁-N₃/O₀-O₂/S₀—S₁-nitrogenheterocyclyl residues are optionally substituted with one to three substituents selected from R^{N1}, R^{N2}, Z¹, Z², Z³, Z⁵, Z⁶ and Z⁷.

Still more preferably R⁸ and R⁹ represents independently of each other the following spironitrogencyclyl, nitrogenheterocyclyl, C₅-C₁₄/N₁—N₃-spironitrogencyclyl or C₅-C₁₄/N₁-N₃/O₀-O₂/S₀—S₁-nitrogenheterocyclyl residues:
azaspiro[3,3]heptyl linked through the nitrogen atom, azaspiro[3,4]octyl linked through the nitrogen atom, azaspiro[3,5]nonyl linked through the nitrogen atom, azaspiro[3,6]decyl linked through the nitrogen atom, azaspiro[4,4]nonyl linked through the nitrogen atom, azaspiro[4,5]decyl linked through the nitrogen atom, azaspiro[4,6]undecyl linked through the nitrogen atom, azaspiro[5,5]undecyl linked through the nitrogen atom, azaspiro[5,6]dodecyl linked through the nitrogen atom, azaspiro[6,6]tridecyl linked through the nitrogen atom,
substituted azaspiro[3,3]heptyl linked through the nitrogen atom, substituted azaspiro[3,4]octyl linked through the nitrogen atom, substituted azaspiro[3,5]nonyl linked through the nitrogen atom, substituted azaspiro[3,6]decyl linked through the nitrogen atom, substituted azaspiro[4,4]nonyl linked through the nitrogen atom, substituted azaspiro[4,5]decyl linked through the nitrogen atom, substituted azaspiro[4,6]undecyl linked through the nitrogen atom, substituted azaspiro[5,5]undecyl linked through the nitrogen atom, substituted azaspiro[5,6]dodecyl linked through the nitrogen atom, substituted azaspiro[6,6]tridecyl linked through the nitrogen atom,
diazaspiro[3,3]heptyl linked through a nitrogen atom, diazaspiro[3,4]octyl linked through a nitrogen atom, diazaspiro[3,5]nonyl linked through a nitrogen atom, diazaspiro[3,6]decyl linked through a nitrogen atom, diazaspiro[4,4]nonyl linked through a nitrogen atom, diazaspiro[4,5]decyl linked through a nitrogen atom, diazaspiro[4,6]undecyl linked through a nitrogen atom, diazaspiro[5,5]undecyl linked through a nitrogen atom, diazaspiro[5,6]dodecyl linked through a nitrogen atom,
diazaspiro[6,6]tridecyl linked through a nitrogen atom, substituted diazaspiro[3,3]heptyl linked through a nitrogen atom, substituted diazaspiro[3,4]octyl linked through a nitrogen atom, substituted diazaspiro[3,5]nonyl linked through a nitrogen atom, substituted diazaspiro[3,6]decyl linked through a nitrogen atom, substituted diazaspiro[4,4]nonyl linked through a nitrogen atom, substituted diazaspiro[4,5]decyl linked through a nitrogen atom, substituted diazaspiro[4,6]undecyl linked through a nitrogen atom, substituted diazaspiro[5,5]undecyl linked through a nitrogen atom, substituted diazaspiro[5,6]dodecyl linked through a nitrogen atom, substituted diazaspiro[6,6]tridecyl linked through a nitrogen atom,
triazaspiro[3,5]nonyl linked through a nitrogen atom, triazaspiro[3,6]decyl linked through a nitrogen atom, triazaspiro[4,5]decyl linked through a nitrogen atom, triazaspiro[4,6]undecyl linked through a nitrogen atom, triazaspiro[5,5]undecyl linked through a nitrogen atom, triazaspiro[5,6]dodecyl linked through a nitrogen atom, triazaspiro[6,6]tridecyl linked through a nitrogen atom,
substituted triazaspiro[3,5]nonyl linked through a nitrogen atom, substituted triazaspiro[3,6]decyl linked through a nitrogen atom, substituted triazaspiro[4,5]decyl linked through a nitrogen atom, substituted triazaspiro[4,6]undecyl linked through a nitrogen atom, substituted triazaspiro[5,5]undecyl linked through a nitrogen atom, substituted triazaspiro[5,6]dodecyl linked through a nitrogen atom, substituted triazaspiro[6,6]tridecyl linked through a nitrogen atom,
oxazaspiro[3,3]heptyl linked through a nitrogen atom, oxazaspiro[3,4]octyl linked through a nitrogen atom, oxazaspiro[3,5]nonyl linked through a nitrogen atom, oxazaspiro[3,6]decyl linked through a nitrogen atom, oxazaspiro[4,4]nonyl linked through a nitrogen atom, oxazaspiro[4,5]decyl linked through a nitrogen atom, oxazaspiro[4,6]undecyl linked through a nitrogen atom, oxazaspiro[5,5]undecyl linked through a nitrogen atom, oxazaspiro[5,6]dodecyl linked through a nitrogen atom, oxazaspiro[6,6]tridecyl linked through a nitrogen atom,
substituted oxazaspiro[3,3]heptyl linked through a nitrogen atom, substituted oxazaspiro[3,4]octyl linked through a nitrogen atom, substituted oxazaspiro[3,5]nonyl linked through a nitrogen atom, substituted oxazaspiro[3,6]decyl linked through a nitrogen atom, substituted oxazaspiro[4,4]nonyl linked through a nitrogen atom, substituted oxazaspiro[4,5]decyl linked through a nitrogen atom, substituted oxazaspiro[4,6]undecyl linked through a nitrogen atom, substituted oxazaspiro[5,5]undecyl linked through a nitrogen atom, substituted oxazaspiro[5,6]dodecyl linked through a nitrogen atom, substituted oxazaspiro[6,6]tridecyl linked through a nitrogen atom,
oxadiazaspiro[3,5]nonyl linked through a nitrogen atom, oxadiazaspiro[3,6]decyl linked through a nitrogen atom, oxadiazaspiro[4,5]decyl linked through a nitrogen atom, oxadiazaspiro[4,6]undecyl linked through a nitrogen atom, oxadiazaspiro[5,5]undecyl linked through a nitrogen atom, oxadiazaspiro[5,6]dodecyl linked through a nitrogen atom, oxadiazaspiro[6,6]tridecyl linked through a nitrogen atom,
substituted oxadiazaspiro[3,5]nonyl linked through a nitrogen atom, substituted oxadiazaspiro[3,6]decyl linked through a nitrogen atom, substituted oxadiazaspiro[4,5]decyl linked through a nitrogen atom, substituted oxadiazaspiro[4,6]undecyl linked through a nitrogen atom, substituted oxadiazaspiro[5,5]undecyl linked through a nitrogen atom, substituted oxadiazaspiro[5,6]dodecyl linked through a nitrogen atom, substituted oxadiazaspiro[6,6]tridecyl linked through a nitrogen atom, wherein the afore-mentioned substituted spironitrogencyclyl, substituted nitrogenheterocyclyl, substituted C₅-C₁₄/N₁—N₃-spironitrogencyclyl or substituted C₅-C₁₄/N₁-N₃/O₀-O₂/S₀—S₁-nitrogenheterocyclyl residues are optionally substituted with one to three substituents selected from R^{N1}, R^{N2}Z⁵, Z⁶ and Z⁷. Moreover the afore-mentioned substituted or non-substituted spironitrogencyclyl, substituted or non-substituted nitrogenheterocyclyl, substituted or non-substituted C₅-C₁₄/N₁—N₃-spironitrogencyclyl or substituted or non-substituted C₅-C₁₄/N₁-N₃/O₀-O₂/S₀—S₁-nitrogenheterocyclyl residues contain at least one nitrogen atom through which these residues are linked through the rest of the molecule and may contain one or two further moieties selected from oxygen (—O—), sulfoxide (—SO—), sulfone (—SO₂—), carbonyl (—CO—) and nitrogen (—NR^{N2}—).

Still more preferably Ra and R⁹ represents independently of each other the following spironitrogencyclyl, nitrogenheterocyclyl, C₅-C₁₄/N₁—N₃-spironitrogencyclyl or C₅-C₁₄/N₁-N₃/O₀-O₂/S₀—S₁-nitrogen heterocyclyl residues:

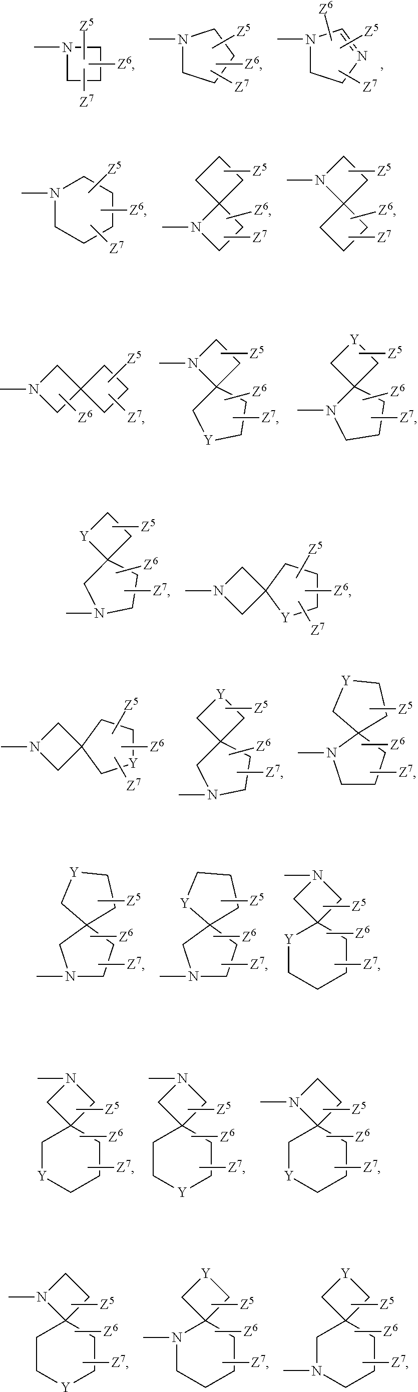

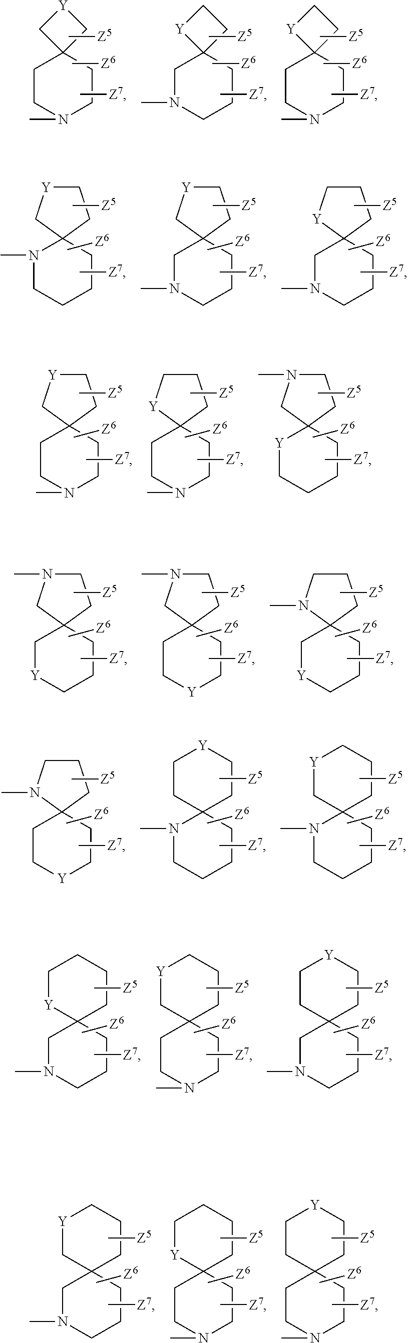

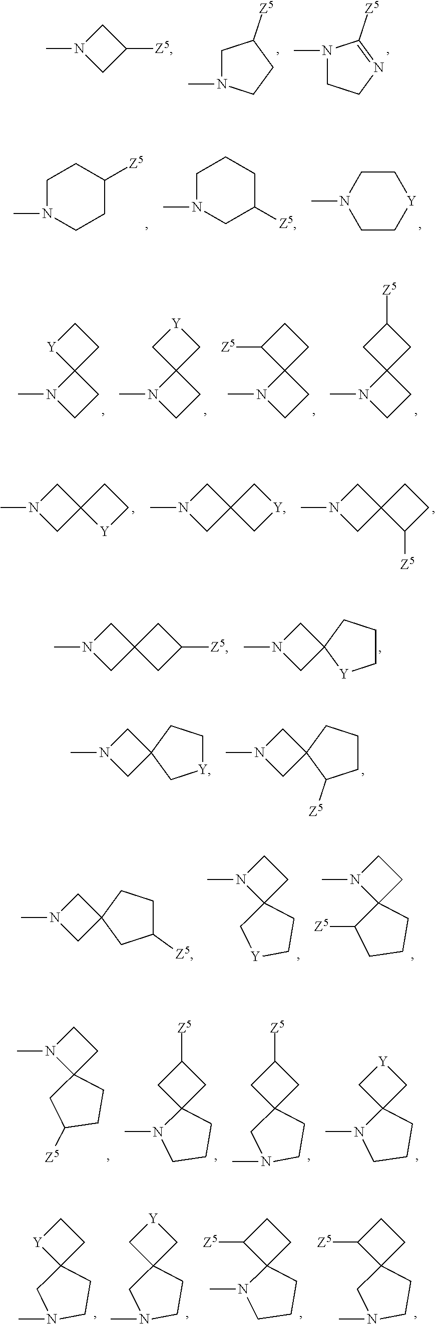

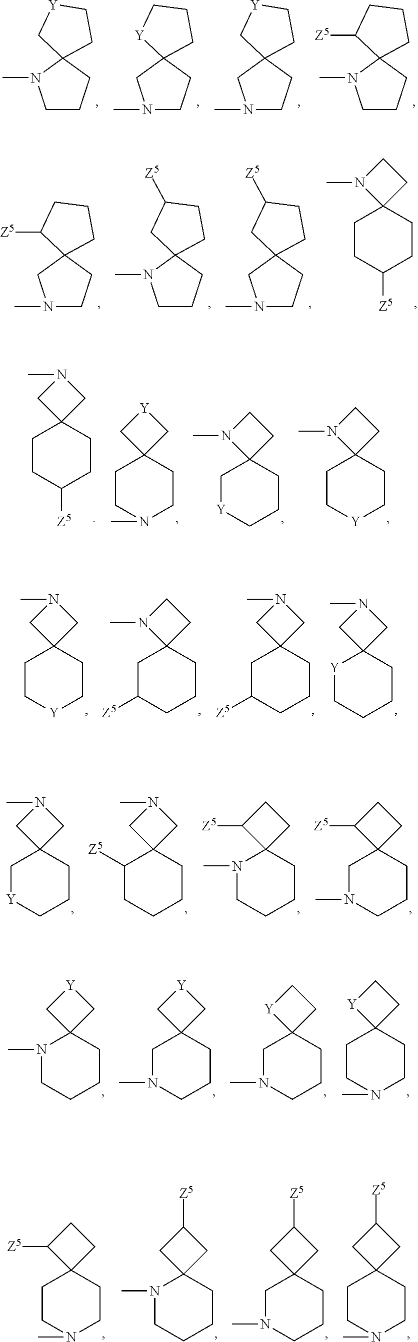

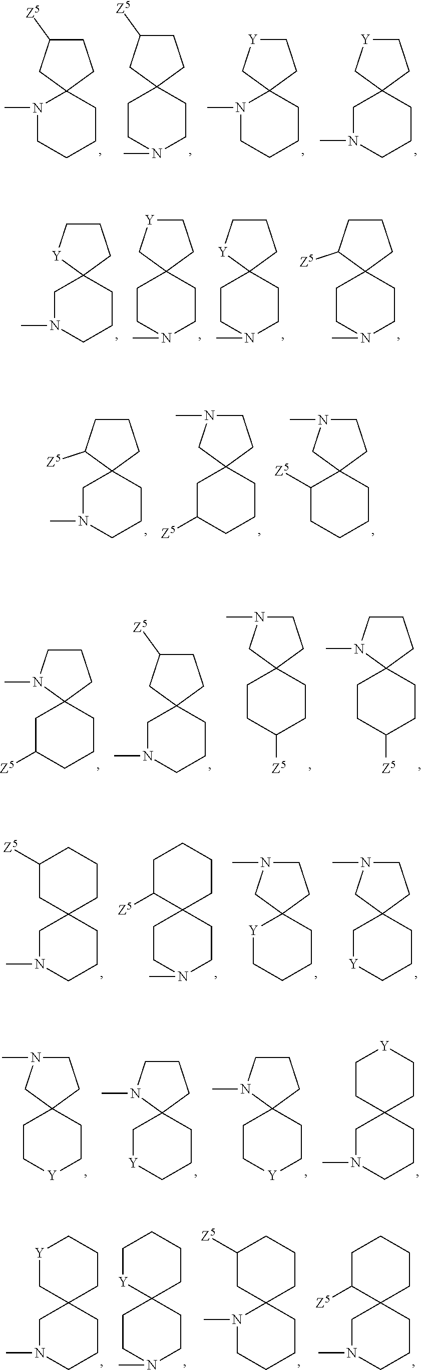

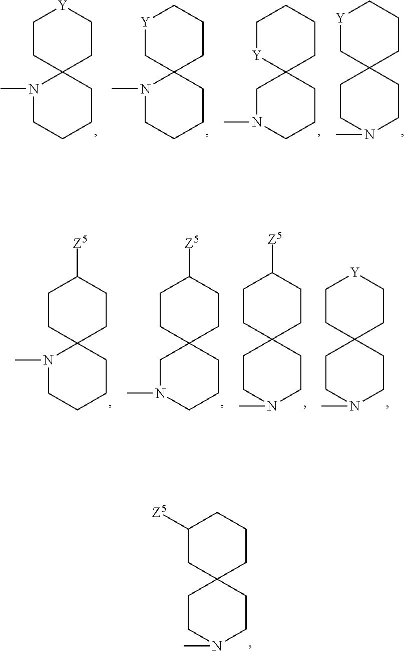

wherein Y represents —O—, —NH—, —NR^{N1}—, —NR^{N2}—, —SO—, or —O₂—, preferably —NH— or —NR^{N1}— and wherein the substituents Z⁵, Z⁶ and Z⁷ have the meanings as defined herein. the substituents Z⁵, Z⁶ and Z⁷ may be replaced by the substituents Z¹, Z² and Z³.

Preferably Z⁵, Z⁶ and Z⁷ represent independently of each other —H, —CH₃, —C₂H₅, —C₃H₇, —CH(CH₃)₂, —OH, —OCH₃, —OC₂H₅, —OC₃H₇, —NH₂, —NH(CH₃), —N(CH₃)₂, —F, —Cl, —Br, —I, —CN, —CH₂F, —CHF₂, —CF₃, —OCHF₂, or —OCF₃, more preferably —NH₂, —NH(CH₃), or —N(CH₃)₂.

Preferred is the compound of formula (I), wherein R⁸ and R⁹ represent independently of each other:

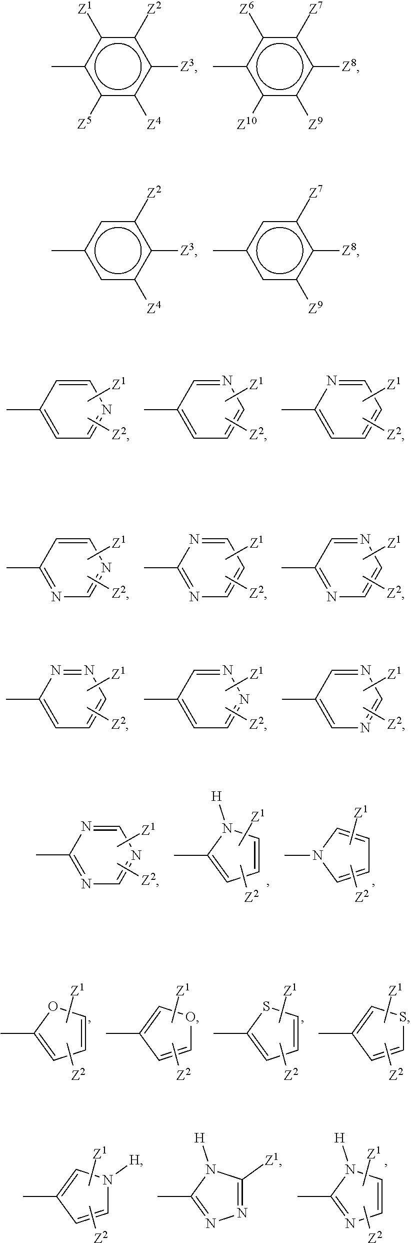

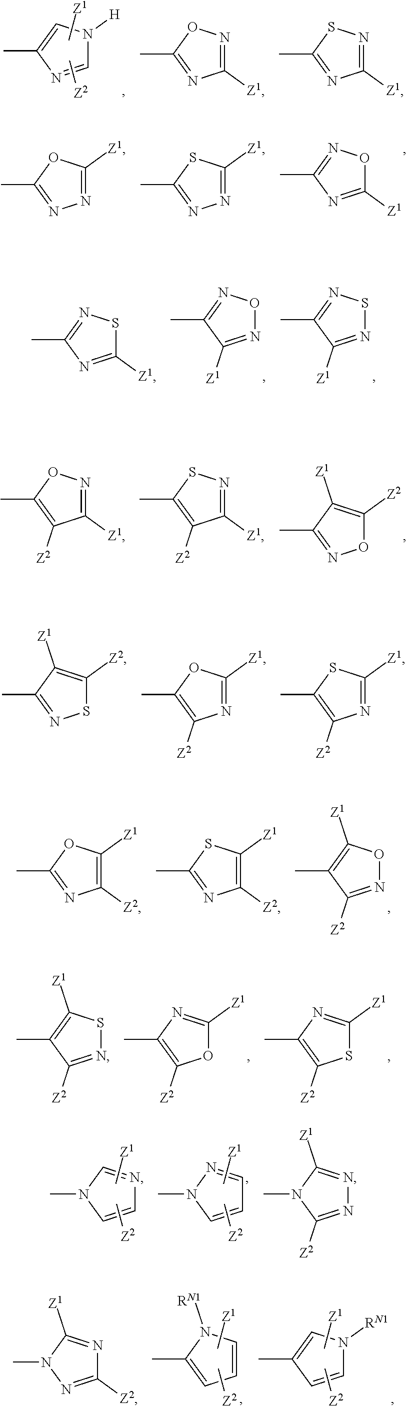

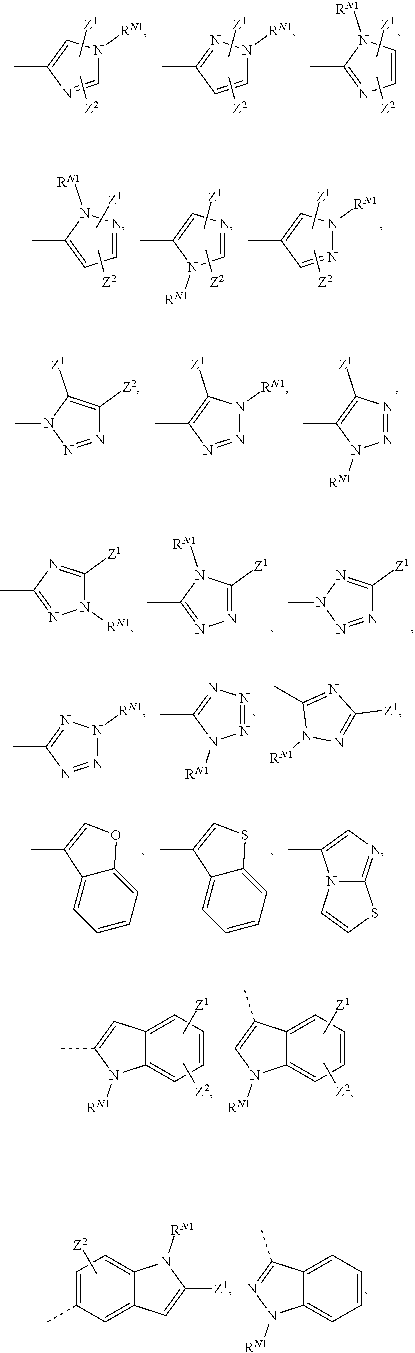

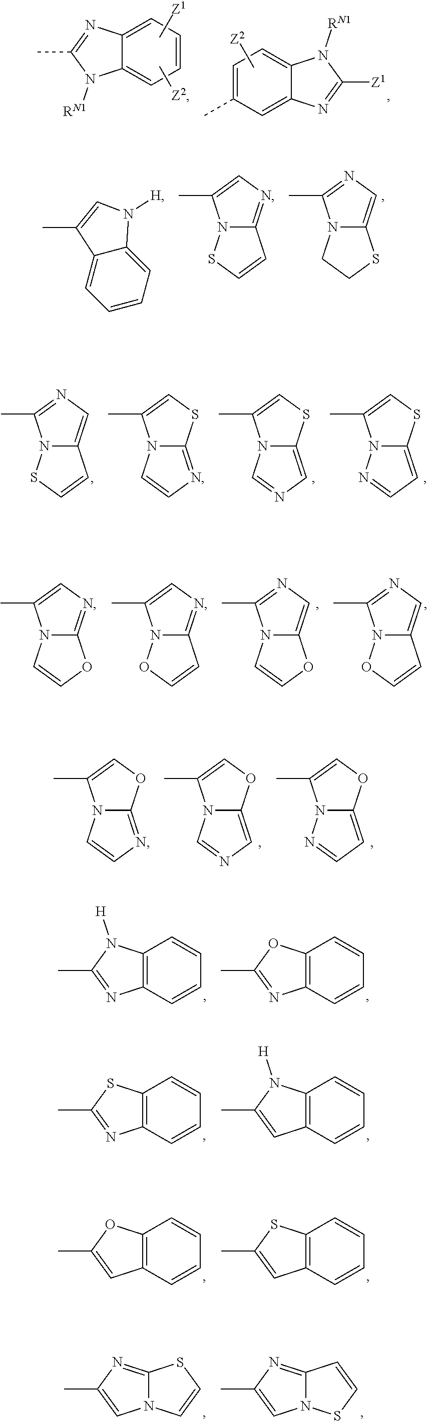

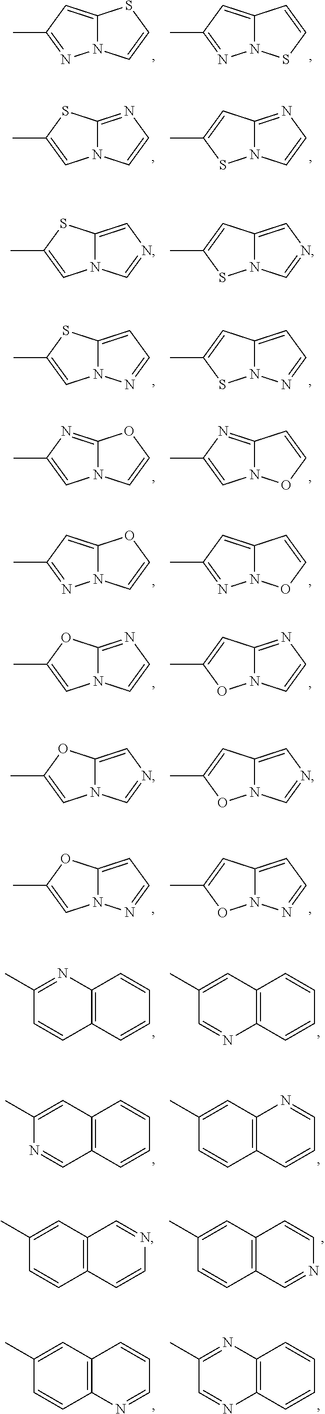

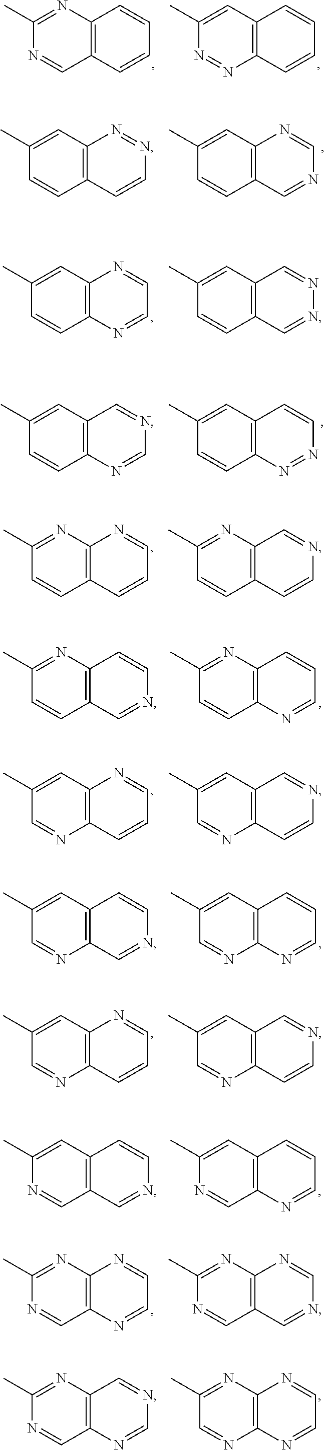

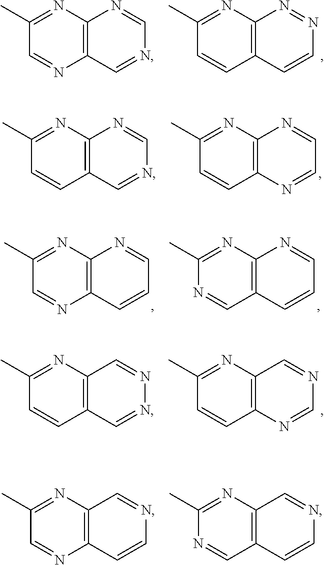

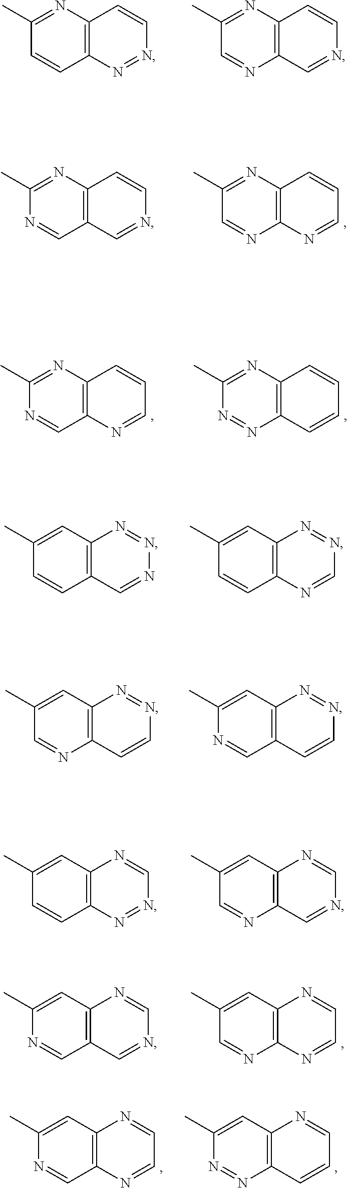

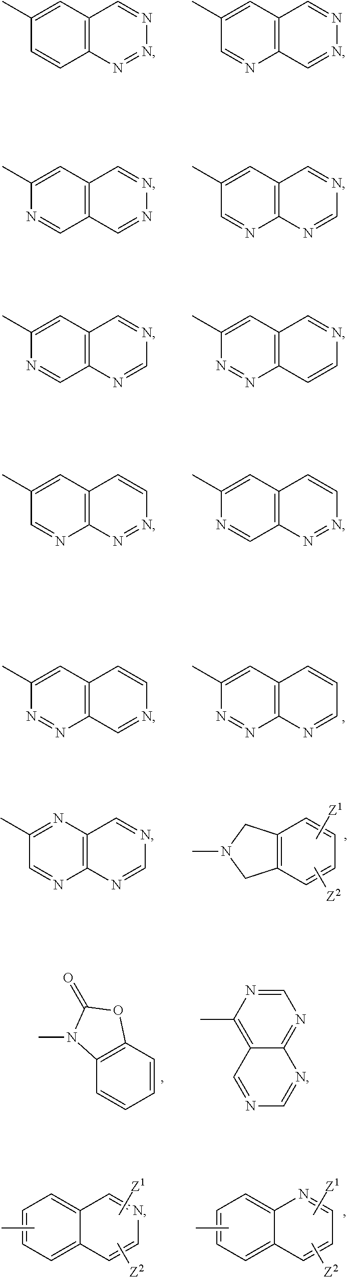

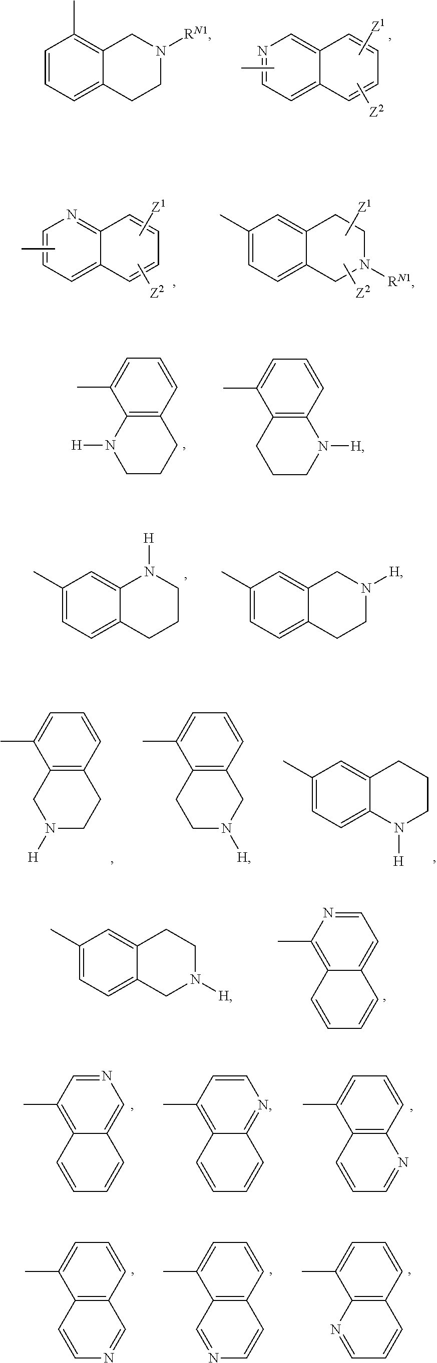

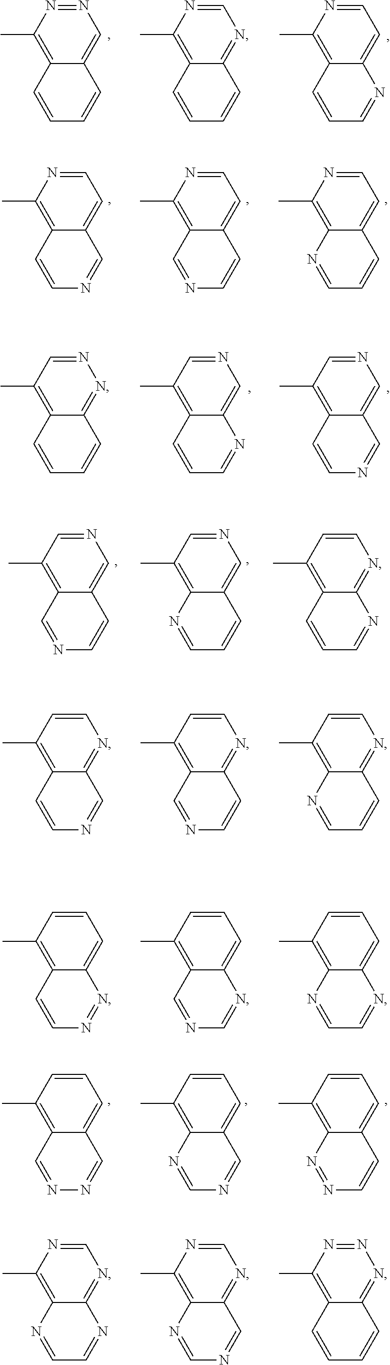

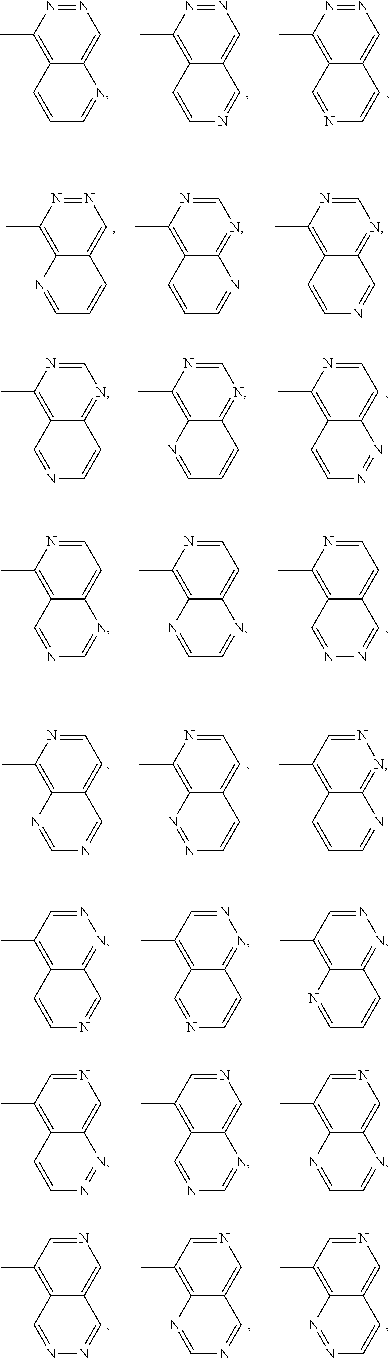

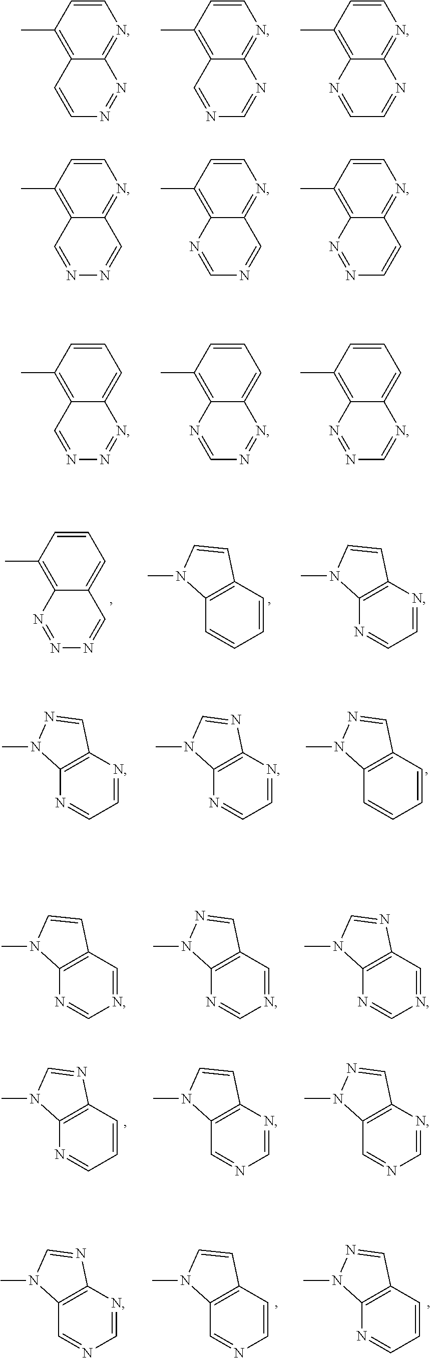

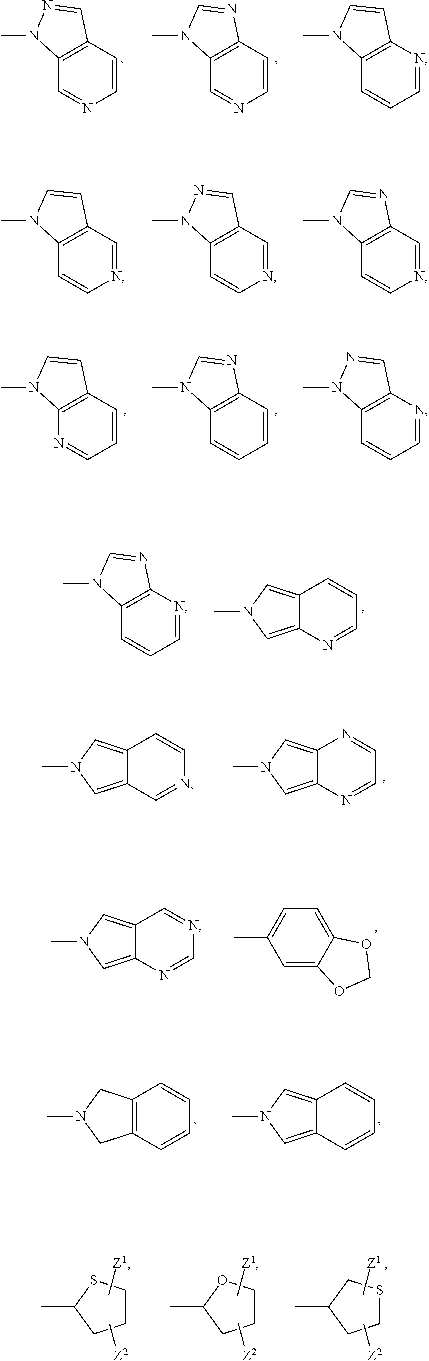

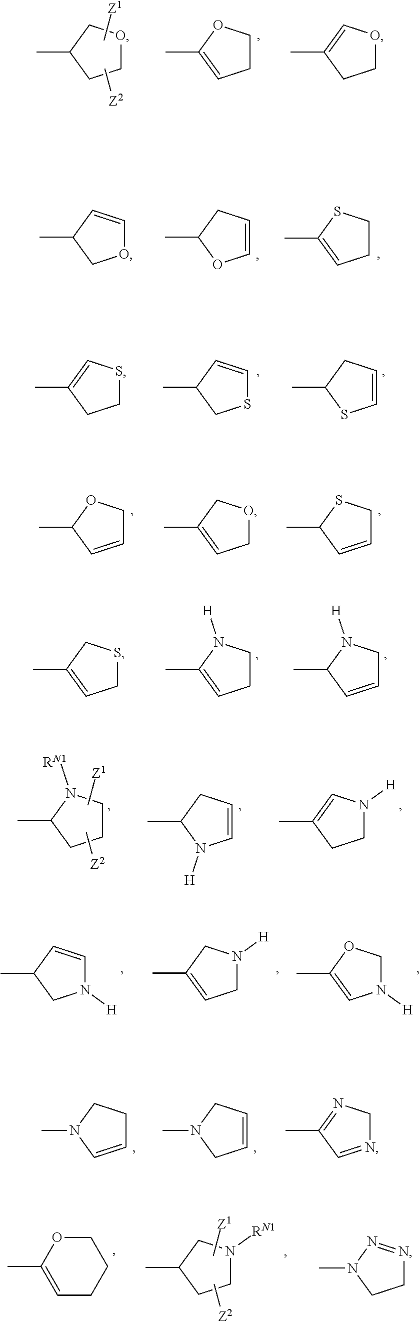

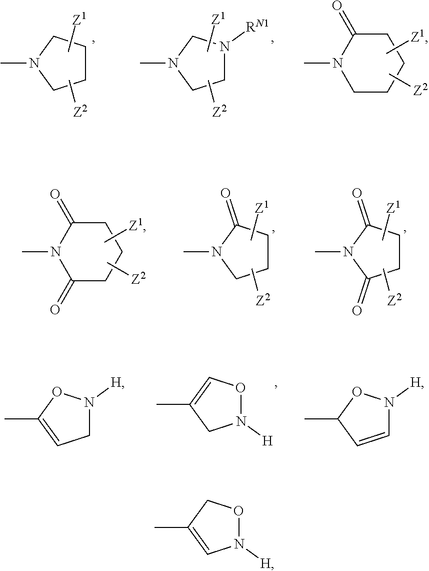

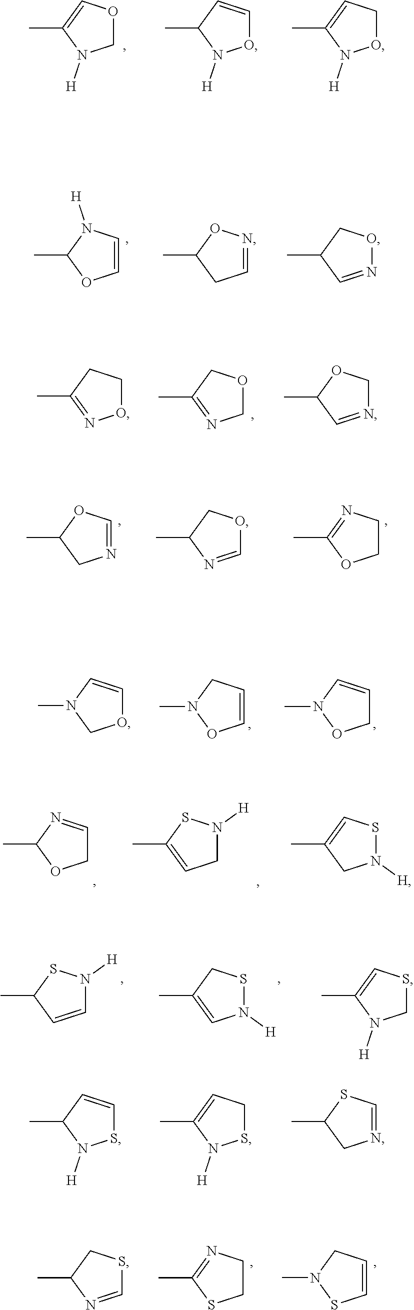

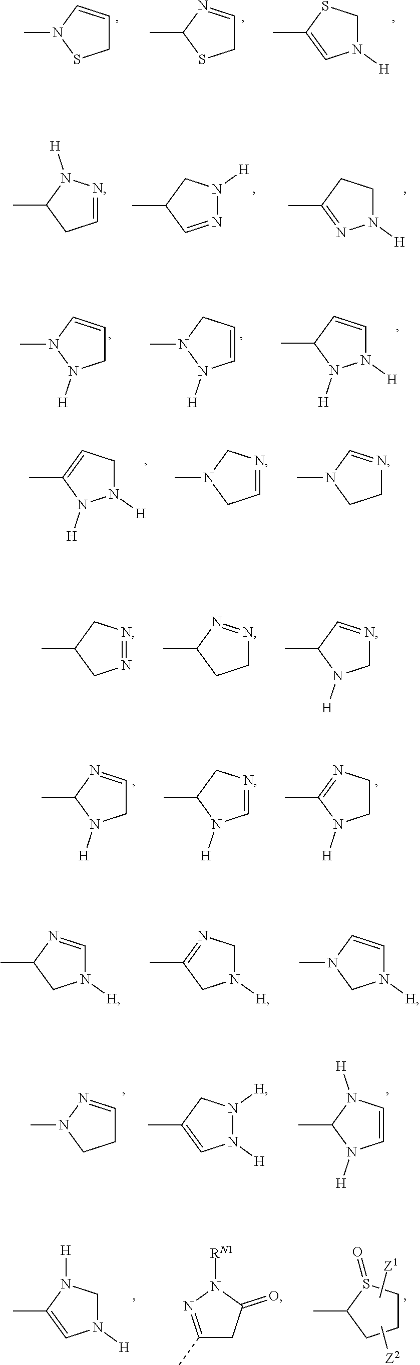

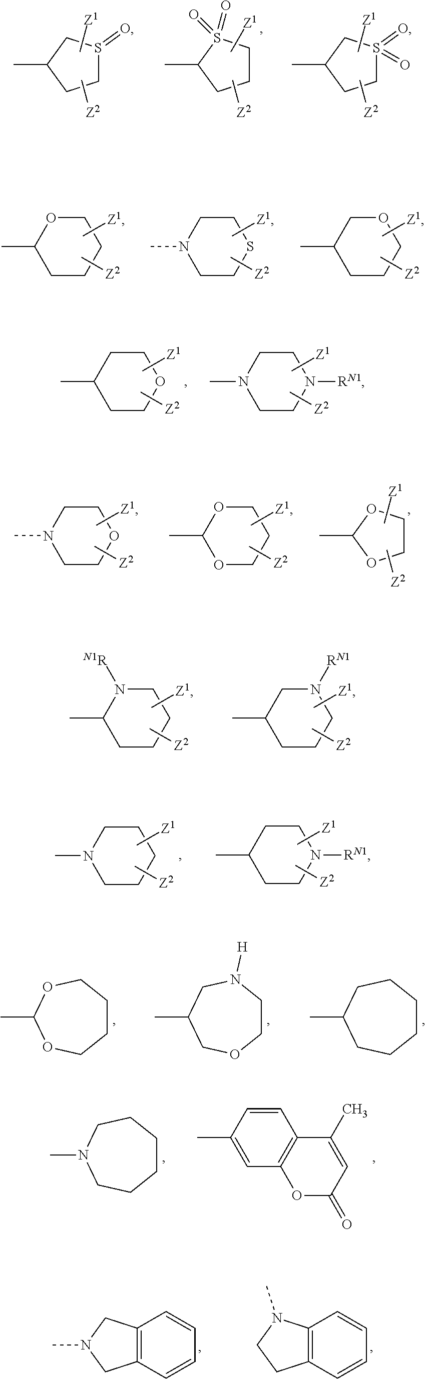

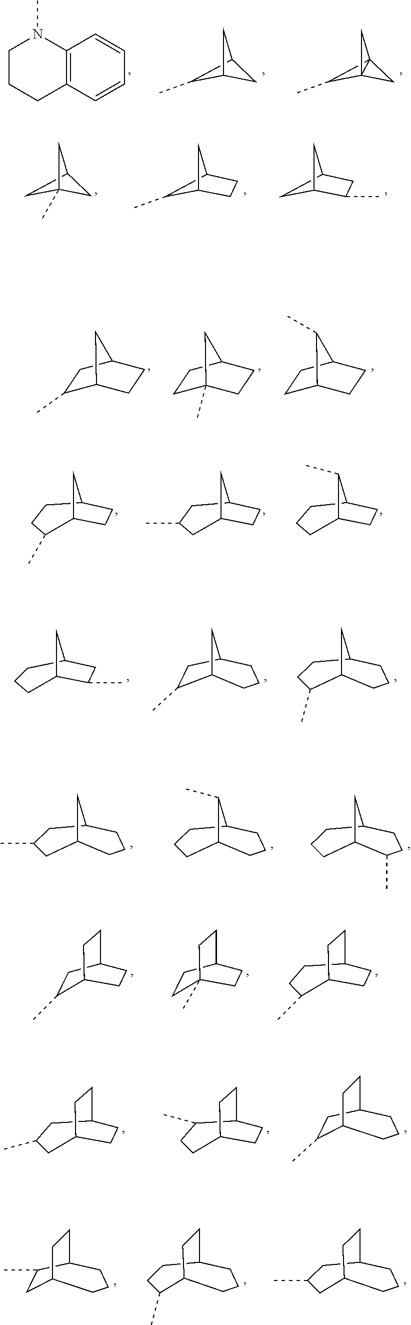

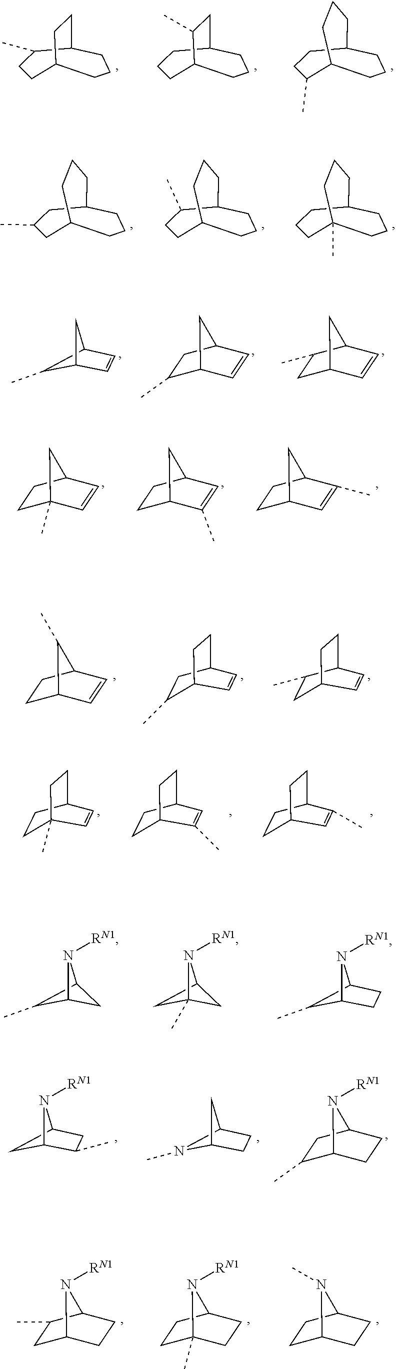

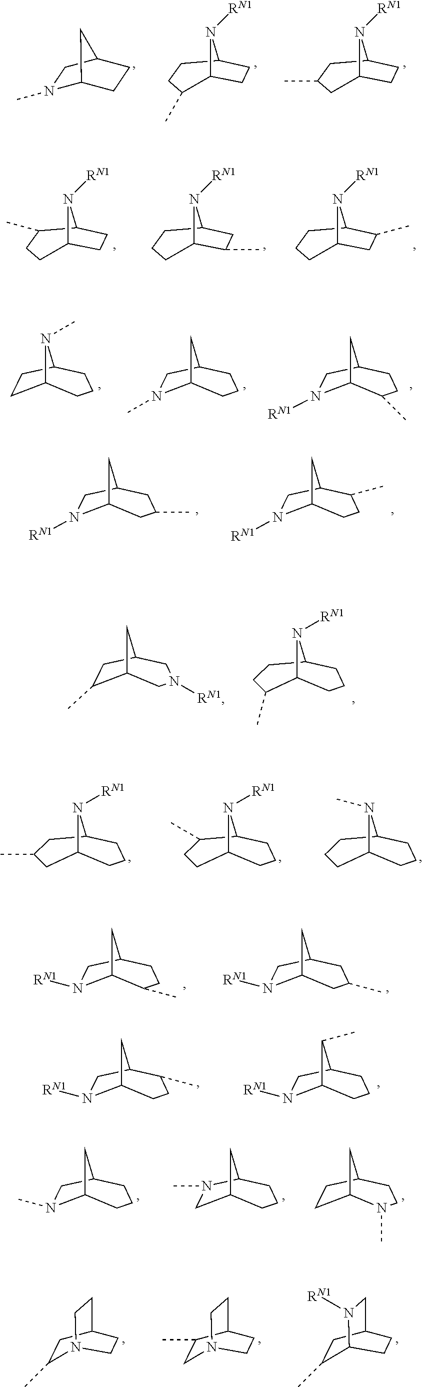

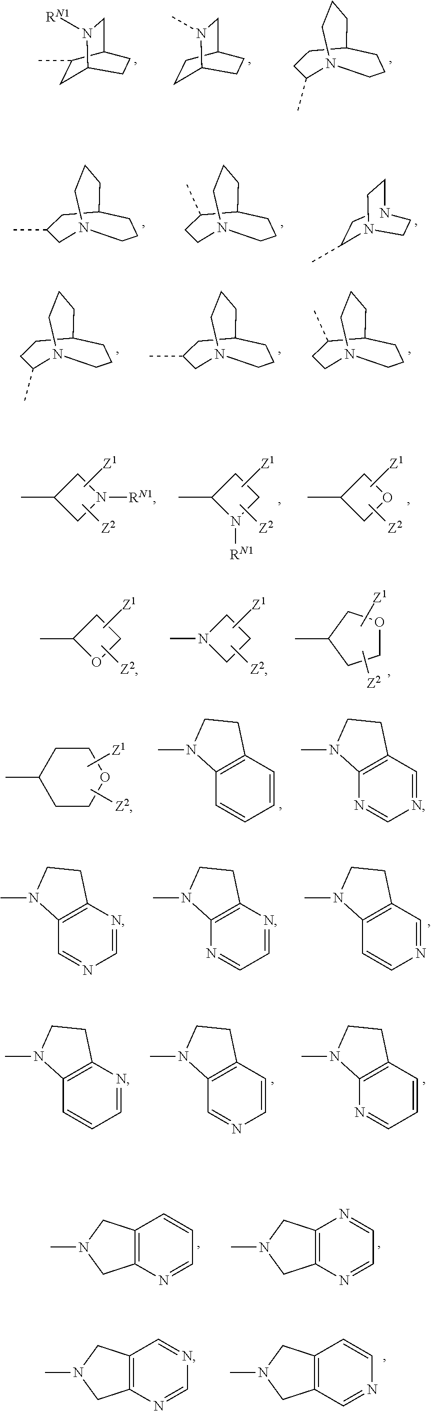

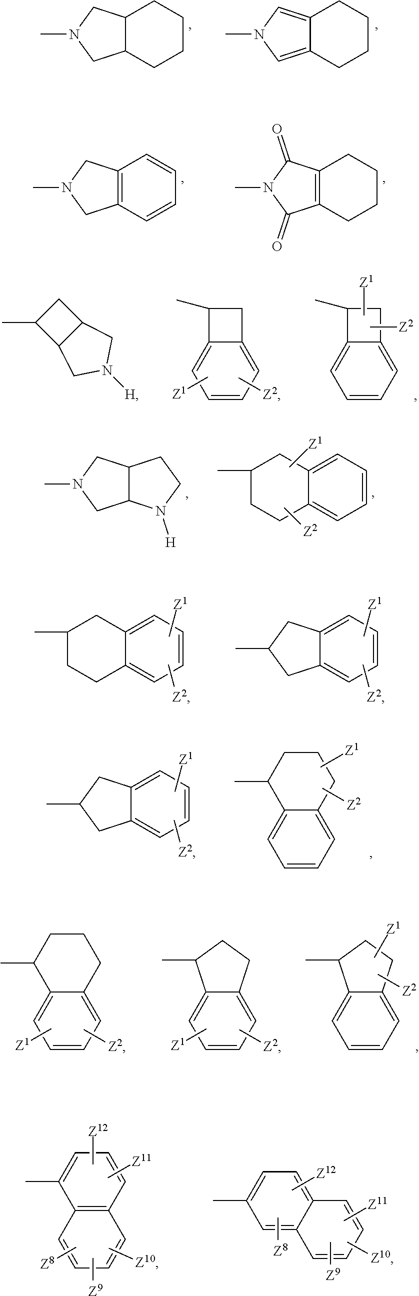

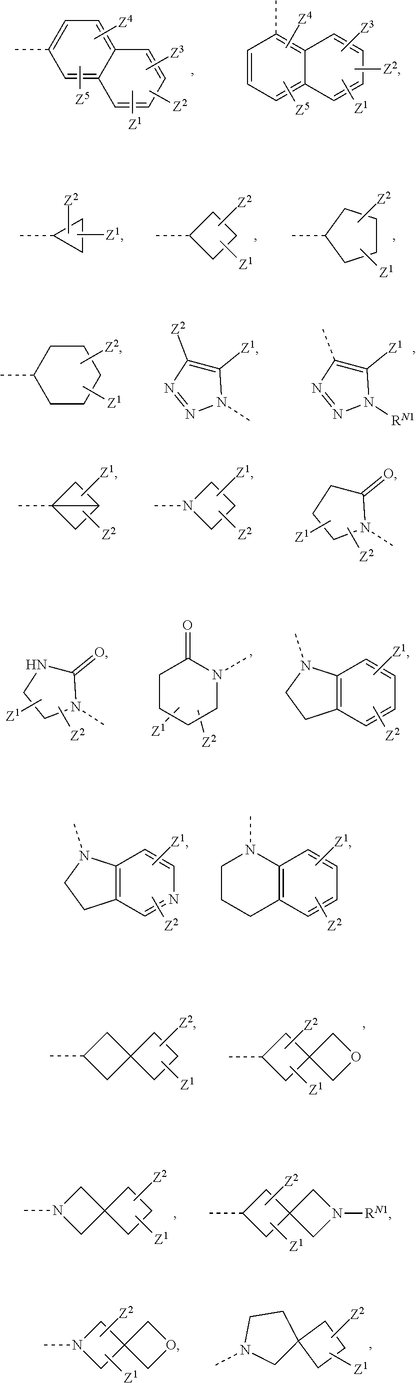

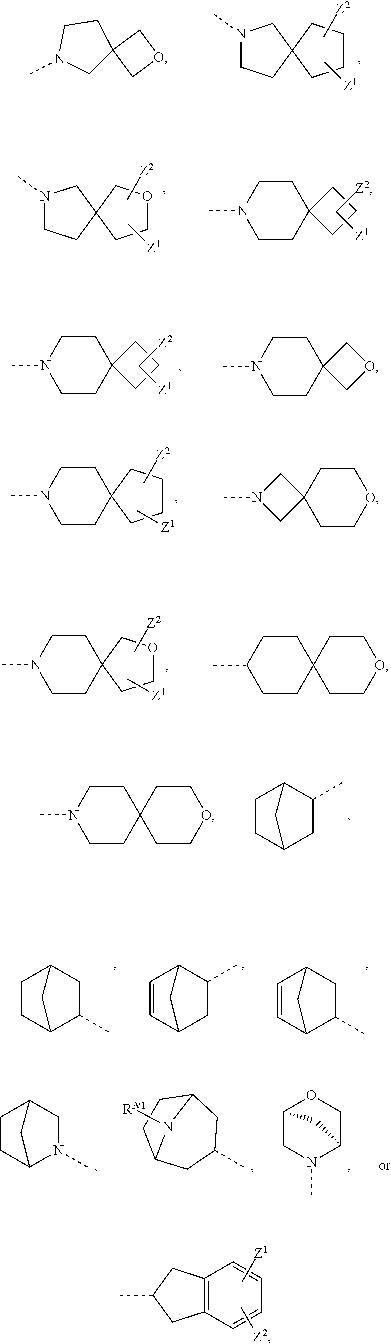

wherein R^{N1}, Z¹, Z², Z³, Z⁴, Z⁵, Z⁶, Z⁷, Z⁸, Z⁹, Z¹⁰, Z¹¹, and Z¹² have the same meanings as defined in formula (I).

More preferred is the compound of formula (I), wherein R⁸ and R⁹ represent independently of each other:

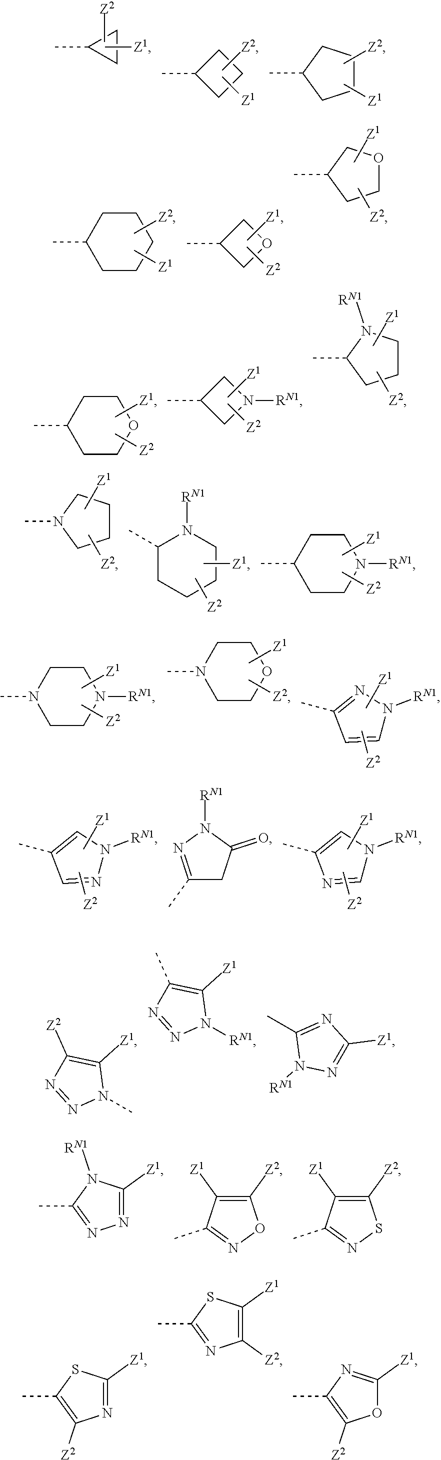

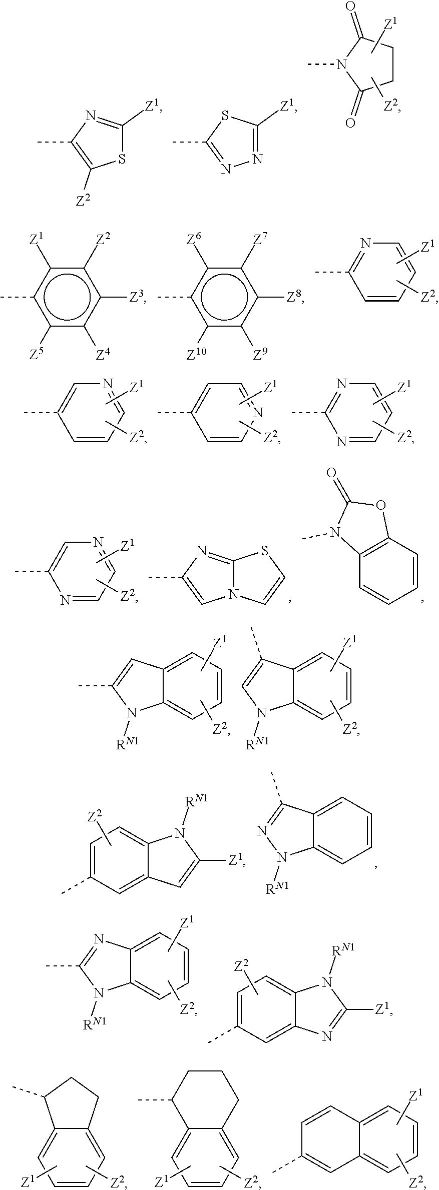

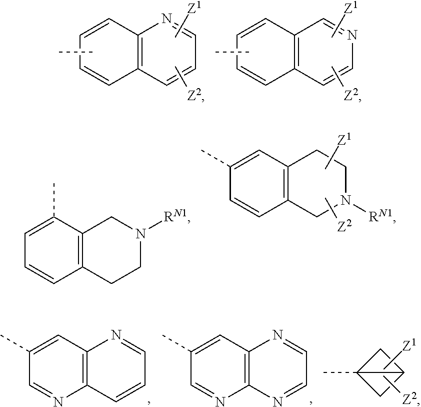

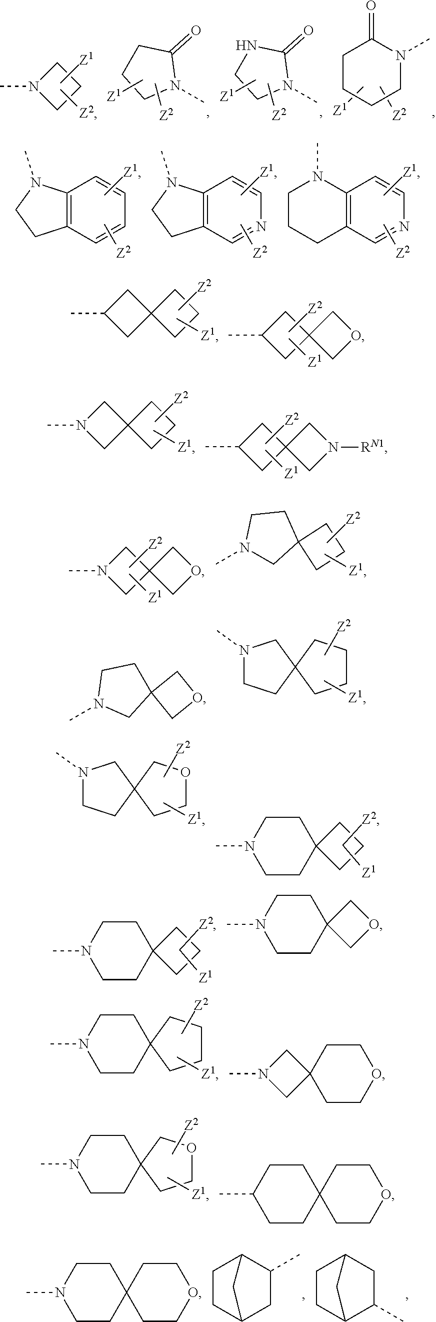

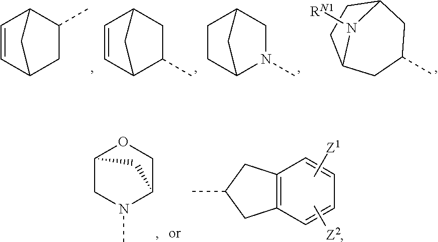

wherein R^{N1}, Z¹, Z², Z³, Z⁴, Z⁵, Z⁶, Z⁷, Z⁸, Z⁹, and Z¹⁰ have the same meanings as defined in formula (I).

Preferred are the compounds of the general formula (I)

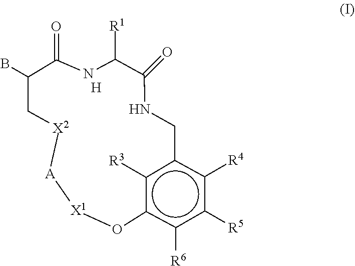

wherein
A represents —CO—N(R^{N6})—,

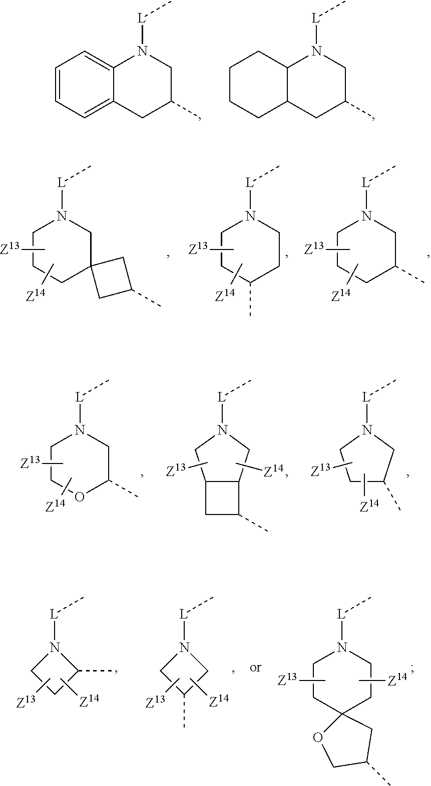

preferably —CO—NH—, A represents —CO—N(CH₃)—,

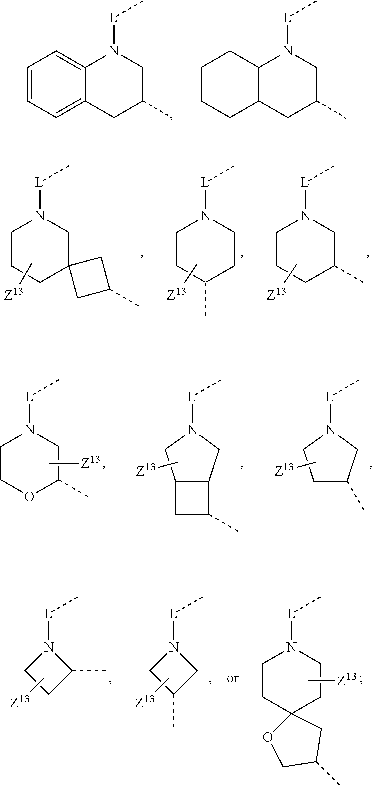

B represents —H, —NH(R²),

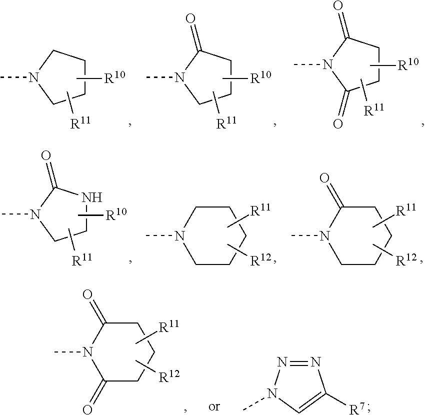

preferably B represents —H, —NH(R²),

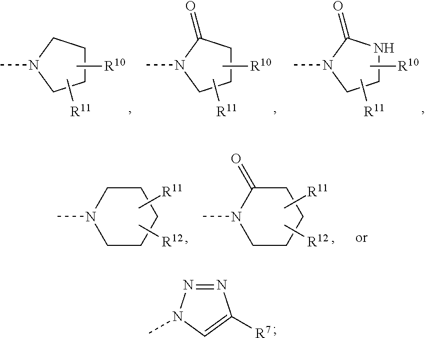

L represents —CO—, —CO—NH—, —CO—N(CH₃)—, or —CO—O—;
R¹ represents —H, —(CH₂)ₚ—R⁷, —(CH₂)ₚ—NH—R⁷, —(CH₂)ₚ—R⁹, or —(CH₂)ₚ—N(Ph)-R⁹; and preferably —(CH₂)ₚ—R⁷, —(CH₂)ₚ—R⁹, or —(CH₂)ₚ—N(Ph)-R⁹;
R² represents —H, —R⁸, —R¹¹, -L¹-R¹¹, -L¹-(CH₂)ᵣ—R⁸, -L¹-R¹⁰, -L¹-(C₂H₄O)ₛ—R¹¹, -L¹-(CH₂)ₜ—O—R¹¹, -L¹-(CH₂)ₜ—NH—(CH₂)ᵣ—R⁸, -L¹-(CH₂)ₜ—O—(CH₂)ᵣ—R⁸, -L¹-(CH₂)ₜ—NHR⁸, -L¹-(CH₂)ₜ—NH—CO—R⁸, -L¹-(CH₂)ₜ—NH—SO₂—R⁸, —CO—(CH₂)ₜ—N(CH₃)R¹⁰, —CO—(CH₂)ₜ—O—(CH₂)ᵤ—N(CH₃)R¹⁰, -L¹-(CH₂)ᵣ—R¹⁴, —CO—C(R¹²)(R¹³)—R¹⁰, —CO—C(R¹²)(R¹³)—R⁸, or —CO—C(R¹²)(R¹³)—(CH₂)ᵤ—R⁸;
L¹ represents a bond, —CO—, —CO₂—, —CONH—, or —SO₂—;
R³ represents —H;
R⁴-R⁶ represent independently of each other —H, —CH₃, or —OCH₃; and more preferably —H or —CH₃;
R⁵ and R⁶ may form together

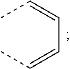

R⁸ and R⁹ represent independently of each other
C₆-C₁₄ aryl, C₁-C₁ heteroaryl, C₃-C₈ carbocyclyl, C₁-C₉ heterocyclyl, C₄-C₁₁ bicyclic carbocyclyl, C₄-C₁₁ bridged carbocyclyl, C₁-C₁ bicyclic heterocyclyl, C₁-C₁₀ bridged heterocyclyl, C₇-C₁₆-spiroalkyl, C₅-C₁₄-spiroheterocyclyl,
wherein all afore-mentioned ring systems can be substituted with 1 to 5 substituents selected from Z¹, Z², Z³, Z⁴, Z⁵, Z⁶, Z⁷, Z⁸, Z⁹, Z¹⁰, Z¹¹, Z¹², R^{N1} and R^{N2}; and C₁-C₁₀ heteroaryl, C₁-C₉ heterocyclyl, C₁-C₁ bicyclic heterocyclyl, C₁-C₁ bridged heterocyclyl, C₅-C₁₄-spiroheterocyclyl ring systems contain at least one of heteroatoms N, O, and S;
said C₃-C₈ carbocyclyl, C₁-C₉ heterocyclyl, C₁-C₁ bicyclic heterocyclyl, C₄-C₁₁ bridged carbocyclyl, C₁-C₁ bicyclic heterocyclyl, C₁-C₁ bridged heterocyclyl, C₇-C₁₆-spiroalkyl, C₅-C₁₄-spiroheterocyclyl ring systems can be partly saturated or unsaturated;
R⁷ and R¹⁰ represent independently of each other —H, —CH₃, —C₂H₅, —C₃H₇, —CH(CH₃)₂, —CH₂F, —CHF₂, —CF₃, —CH₂CN, —C(CH₃)₂—CN, —CH₂—C(CH₃)₂—CN, —CH₂—CF₃, —CH₂—C(CH₃)₂—NH₂,

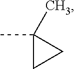

cyclo-C₃H₅, cyclo-C₄H₇, cyclo-C₅H₉, cyclo-C₆H₁₁, —C₄H₉, —CH₂—CH(CH₃)₂, —CH(CH₃)—C₂H₅, —C(CH₃)₃, —C₅H₁₁, —CH₂—C(CH₃)₃, —CH(C₂H₅)₂, —C₂H₄—CH(CH₃)₂, -Ph, —CH₂-Ph, —CH₂—CH₂-Ph, —C₂H₄—CH═CH₂, —CH₂—CH═CH—CH₃, —CH₂—C(CH₃)=CH₂, —CH(CH₃)—CH═CH₂, —C(CH₃)=CH—CH₃, —CH₂—CH═C(CH₃)₂, —CO—CH═C(CH₃)₂, —CH₂—C≡CH, —C₂H₄—C≡CH, —CH₂—C≡C—CH₃, —CH₂—OCF₃, —C₂H₄—OCF₃, —C₃H₆—OCF₃, —CH₂—OCHF₂, —C₂H₄—OCHF₂, —C₃H₆—OCHF₂, —CH₂—OCH₃, —C₂H₄—OCH₃, —C₃H₆—OCH₃, —CH₂—OC₂H₅, —C₂H₄—OC₂H₅, —C₃H₆—OC₂H₅, —CH₂—OH, —C₂H₄—OH, —C₃H₆—OH, —CH₂—COOH, —C₂H₄—COOH, —C₃H₆—COOH, —C(CH₃)₂—CN, —C(CH₃)₂—OH, —C(CH₃)₂—CH₂—OH, —C(C₂H₅)₂—CH₂—OH, —C(CH₂—OH)₂—CH₃, —C(CH₂—OH)₂—C₂H₅, —C(CH₃)₂—CH₂—SH, —C(C₂H₅)₂—CH₂—SH, —C(CH₂—SH)₂—CH₃, —CO—O—C(CH₃)₃, or —C(CH₂—SH)₂—C₂H₅;
R¹¹ represents —H, —CH₃, —C₂H₅, —C₃H₇, —CH(CH₃)₂, —CH₂F, —CHF₂, —CF₃, —CH₂CN, —C(CH₃)₂—CN, —CH₂—C(CH₃)₂—CN, —CH₂—CF₃, —CH₂—C(CH₃)₂—NH₂,

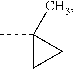

cyclo-C₃H₅, cyclo-C₄H₇, cyclo-C₅H₉, cyclo-C₆H₁₁, —C₄H₉, —CH₂—CH(CH₃)₂, —CH(CH₃)—C₂H₅, —C(CH₃)₃, —C₅H₁₁, —CH₂—C(CH₃)₃, —CH(C₂H₅)₂, -Ph, —CH₂-Ph, —CH₂—CH₂-Ph, —CH═CH₂, —CH₂—CH═CH₂, —C(CH₃)=CH₂, —CH═CH—CH₃, —C(CH₃)=CH—CH₃, —CH═C(CH₃)₂, —C(CH₃)═C(CH₃)₂, —C₂H₄—CH═CH₂, —CH₂—CH═CH—CH₃, —CH₂—CH═C(CH₃)₂, —C≡CH, —CH₂—C≡CH, —C₂H₄—C≡CH, —CH₂—C≡C—CH₃, —C≡C—CH₃, —C≡C—C₂H₅, —CH₂—OCF₃, —C₂H₄—OCF₃, —C₃H₆—OCF₃, —CH₂—OCHF₂, —C₂H₄—OCHF₂, —C₃H₆—OCHF₂, —CH₂—OCH₃, —C₂H₄—OCH₃, —C₃H₆—OCH₃, —CH₂—OC₂H₅, —C₂H₄—OC₂H₅, —C₃H₆—OC₂H₅, —CH₂—OH, —C₂H₄—OH, —C₃H₆—OH, —C(CH₃)₂—CH₂—OH, —C(C₂H₅)₂—CH₂—OH, —C(CH₂—OH)₂—CH₃, —C(CH₂—OH)₂—C₂H₅, —C(CH₃)₂—CH₂—SH, —C(C₂H₅)₂—CH₂—SH, —C(CH₂—SH)₂—CH₃, or —C(CH₂—SH)₂—C₂H₅;
R¹² and R¹³ represent independently of each other —H, —CH₃, —C₂H₅, —C₃H₇, -Ph, —CH₂-Ph, —NH₂, —NHCO₂(CCH₃)₃, —CH₂—NH₂, —CHF₂, —F, —CF₃, —OCF₃, —OCHF₂, —OH, —OCH₃, —OC₂H₅, and —OC₃H₇; or
R¹² and R¹³ may form together

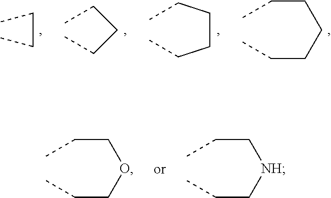

R¹⁴ represents

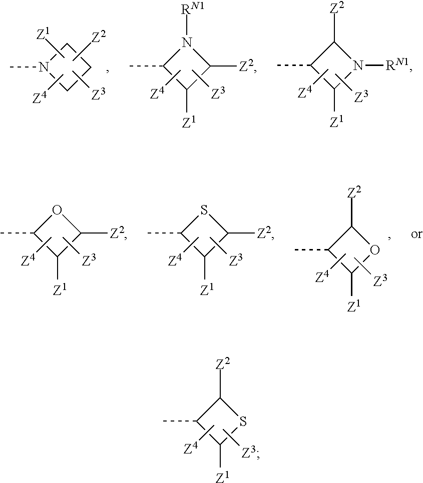

R^{N1}, R^{N2}, and R^{N4} represent independently of each other —H, —CH₃, —C₂H₅, —C₃H₇, —CH(CH₃)₂, —CHF₂, —CF₃,

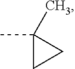

cyclo-C₃H₅, cyclo-C₄H₇, cyclo-C₅H₉, cyclo-C₆H₁₁, —C₄H₉, —CH₂—CH(CH₃)₂, —CH(CH₃)—C₂H₅, —C(CH₃)₃, —CH₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, —C₇H₁₅, —C₈H₁₇, -Ph, —CH₂-Ph, —CH₂—CH₂-Ph, —C₂H₄—CH═CH₂, —CH₂—CH═CH—CH₃, —CH₂—C(CH₃)=CH₂, —CH(CH₃)—CH═CH₂, —CH₂—CH═C(CH₃)₂, —CH₂—C≡CH, —C₂H₄—C≡CH, —CH₂—C≡C—CH₃, —COCH₃, —COC₂H₅, —COC₃H₇, —CO-cyclo-C₃H₅, —COCH(CH₃)₂, —COC(CH₃)₃, —COOH, —COOCH₃, —COOC₂H₅, —COOC₃H₇, —COO-cyclo-C₃H₅, —COOCH(CH₃)₂, —COOC(CH₃)₃, —COOCH₂Ph, —CONH₂, —CONHCH₃, —CONHC₂H₅, —CONHC₃H₇, —CONH-cyclo-C₃H₅, —CONH[CH(CH₃)₂], —CONH[C(CH₃)₃], —CON(CH₃)₂, —CON(C₂H₅)₂, —CON(C₃H₇)₂, —CON(cyclo-C₃H₅)₂, —CON[CH(CH₃)₂]₂, —CON[C(CH₃)₃]₂, —SO₃H, —(CH₂)ᵥ—COOH, —(CH₂)ᵥ—OH, —(CH₂)ᵥ—SH, —(CH₂)ᵥ—SO₃H, or —(CH₂CH₂—O)_{w}—CH₂CH₂—NH₂;
X¹ represents —(CH₂)ₘ—;
X² represents —(CH₂)ₙ—;
X³ represents a bond, —O—, —NH—, or —S—;
Z¹-Z¹⁴ represent independently of each other

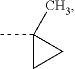

cyclo-C₃H₅, cyclo-C₄H₇, cyclo-C₅H₉, cyclo-C₆H₁₁, —H, —OH, —OCH₃, —OC₂H₅, —OC₃H₇, —O-cyclo-C₃H₅, —OCH(CH₃)₂, —OC(CH₃)₃, —OC₄H₉, —O-cyclo-C₄H₇, —O-cyclo-C₅H₉, —O-cyclo-C₆H₁₁, —OCH₂CH(CH₃)₂, —OCH₂-cyclo-C₃H₅, —OCH₂-cyclo-C₄H₇, —OCH₂-cyclo-C₅H₉, —OCH₂-cyclo-C₆H₁₁, —OPh, —OCH₂-Ph, —OCPh₃, —CH₂—OCH₃, —C₂H₄—OCH₃, —C₃H₆—OCH₃, —CH₂—OC₂H₅, —C₂H₄—OC₂H₅, —C₃H₆—OC₂H₅, —CH₂—OC₃H₇, —C₂H₄—OC₃H₇, —C₃H₆—OC₃H₇, —CH₂—O-cyclo-C₃H₅, —C₂H₄—O-cyclo-C₃H₅, —C₃H₆—O-cyclo-C₃H₅, —CH₂—OCH(CH₃)₂, —C₂H₄—OCH(CH₃)₂, —C₃H₆—OCH(CH₃)₂, —CH₂—OC(CH₃)₃, —C₂H₄—OC(CH₃)₃, —C₃H₆—OC(CH₃)₃, —CH₂—OC₄H₉, —C₂H₄—OC₄H₉, —C₃H₆—OC₄H₉, —CH₂—OPh, —C₂H₄—OPh, —C₃H₆—OPh, —CH₂—OCH₂-Ph, —C₂H₄—OCH₂-Ph, —C₃H₆—OCH₂-Ph, —SH, —SCH₃, —SC₂H₅, —SC₃H₇, —S-cyclo-C₃H₅, —SCH(CH₃)₂, —SC(CH₃)₃, —F, —Cl, —Br, —I, —CN, —COCH₃, —COC₂H₅, —COC₃H₇, —CO-cyclo-C₃H₅, —COCH(CH₃)₂, —COC(CH₃)₃, —COOH, —COOCH₃, —COOC₂H₅, —COOC₃H₇, —COO-cyclo-C₃H₅, —COOCH(CH₃)₂, —COOC(CH₃)₃, —OOC—CH₃, —OOC—C₂H₅, —OOC—C₃H₇, —OOC-cyclo-C₃H₅, —OOC—CH(CH₃)₂, —OOC—C(CH₃)₃, —CONH₂, —CONHCH₃, —CONHC₂H₅, —CONHC₃H₇, —CONH-cyclo-C₃H₅, —CONH[CH(CH₃)₂], —CONH[C(CH₃)₃], —CON(CH₃)₂, —CON(C₂H₅)₂, —CON(C₃H₇)₂, —CON(cyclo-C₃H₅)₂, —CON[CH(CH₃)₂]₂, —CON[C(CH₃)₃]₂, —NHCOCH₃, —NHCOC₂H₅, —NHCOC₃H₇, —NHCO-cyclo-C₃H₅, —NHCO—CH(CH₃)₂, —NHCO—C(CH₃)₃, —NHCO—CH(NH₂)CH₂—COOH, —NHCO—CH(NH₂)CH₂CH₂—COOH, —NHCO—OCH₃, —NHCO—OC₂H₅, —NHCO—OC₃H₇, —NHCO—O-cyclo-C₃H₅, —NHCO—OCH(CH₃)₂, —NHCO—OC(CH₃)₃, —NH₂, —NHCH₃, —NHC₂H₅, —NHC₃H₇, —NH-cyclo-C₃H₅, —NHCH(CH₃)₂, —NHC(CH₃)₃, —N(CH₃)₂, —N(C₂H₅)₂, —N(C₃H₇)₂, —N(cyclo-C₃H₅)₂, —N[CH(CH₃)₂]₂, —N[C(CH₃)₃]₂, —SOCH₃, —SOC₂H₅, —SOC₃H₇, —SO-cyclo-C₃H₅, —SOCH(CH₃)₂, —SOC(CH₃)₃, —SO₂CH₃, —SO₂C₂H₅, —SO₂C₃H₇, —SO₂-cyclo-C₃H₅, —SO₂CH(CH₃)₂, —SO₂C(CH₃)₃, —SO₃H, —SO₃CH₃, —SO₃C₂H₅, —SO₃C₃H₇, —SO₃-cyclo-C₃H₅, —SO₃CH(CH₃)₂, —SO₃C(CH₃)₃, —SO₂NH₂, —SO₂NHCH₃, —SO₂NHC₂H₅, —SO₂NHC₃H₇, —SO₂NH-cyclo-C₃H₅, —SO₂NHCH(CH₃)₂, —SO₂NHC(CH₃)₃, —SO₂N(CH₃)₂, —SO₂N(C₂H₅)₂, —SO₂N(C₃H₇)₂, —SO₂N(cyclo-C₃H₅)₂, —SO₂N[CH(CH₃)₂]₂, —SO₂N[C(CH₃)₃]₂, —O—S(═O)CH₃, —O—S(═O)C₂H₅, —O—S(═O)C₃H₇, —O—S(═O)-cyclo-C₃H₅, —O—S(═O)CH(CH₃)₂, —O—S(═O)C(CH₃)₃, —NH—SO₂—CH₃, —NH—SO₂—C₂H₅, —NH—SO₂—C₃H₇, —NH—SO₂-cyclo-C₃H₅, —NH—SO₂—CH(CH₃)₂, —NH—SO₂—C(CH₃)₃, —O—SO₂—CH₃, —O—SO₂—C₂H₅, —O—SO₂—C₃H₇, —O—SO₂-cyclo-C₃H₅, —O—SO₂—CH(CH₃)₂, —O—SO₂—C(CH₃)₃, —OCH₂F, —OCHF₂, —OCF₃, —CH₂—OCF₃, —C₂H₄—OCF₃, —C₃H₆—OCF₃, —CH₂—OCHF₂, —C₂H₄—OCHF₂, —C₃H₆—OCHF₂, —OC₂F₅, —CH₂—OC₂F₅, —C₂H₄—OC₂F₅, —C₃H₆—OC₂F₅, —NH—CO—NH₂, —NH—CO—NHCH₃, —NH—CO—NHC₂H₅, —NH—CO—NHC₃H₇, —NH—C(═NH)—NH₂, —NH—CO—N(C₃H₇)₂, —NH—CO—NH[CH(CH₃)₂], —NH—CO—NH[C(CH₃)₃], —NH—CO—N(CH₃)₂, —NH—CO—N(C₂H₅)₂, —NH—CO—NH-cyclo-C₃H₅, —NH—CO—N(cyclo-C₃H₅)₂, —NH—CO—N[CH(CH₃)₂]₂, —O—CO—NH-cyclo-C₃H₅, —O—CO—NH[CH(CH₃)₂], —NH—C(═NH)—NH[C(CH₃)₃], —O—CO—NHC₃H₇, —O—CO—NH₂, —O—CO—NHCH₃, —O—CO—NHC₂H₅, —O—CO—NH[C(CH₃)₃], —O—CO—N(CH₃)₂, —O—CO—N(C₂H₅)₂, —O—CO—N(C₃H₇)₂, —O—CO—N(cyclo-C₃H₅)₂, —O—CO—N[CH(CH₃)₂]₂, —O—CO—N[C(CH₃)₃]₂, —CH₂F, —CHF₂, —CF₃, —CH₂—CH₂F, —CH₂—CHF₂, —CH₂—CF₃, cyclo-C₈H₁₅, -Ph, —CH₂-Ph, —CH₂—CH₂-Ph, —CH═CH-Ph, —CH₃, —C₂H₅, —C₃H₇, —CH(CH₃)₂, —C₄H, —CH₂—CH(CH₃)₂, —CH₂—CH₂—CH(CH₃)₂, —CH(CH₃)—C₂H₅, —C(CH₃)₃, —CH₁₁, —C(CH₃)₂—C₂H₅, —CH₂—C(CH₃)₃, —CH(C₂H₅)₂, —C₆H₁₃, —CH═CH₂, —CH₂—CH═CH₂, —C(CH₃)=CH₂, —CH═CH—CH₃, —C₂H₄—CH═CH₂, —CH₂—CH═CH—CH₃, —CH═CH—C₂H₅, —CH₂—C(CH₃)=CH₂, —CH(CH₃)—CH═CH, —CH═C(CH₃)₂, —C(CH₃)=CH—CH₃, —C₃H₆—CH═CH₂, —C₂H₄—CH═CH—CH₃, —C≡CH, —C≡C—CH₃, —CH₂—C≡CH, —C₂H₄—C≡CH, —CH₂—C≡C—CH₃, —C≡C—C₂H₅,

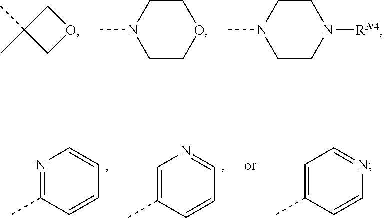

Z³ and Z⁴ may form together or

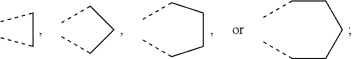

m is an integer selected from 0, 1, 2, or 3;
n is an integer selected from 0, 1, 2, 3, or 4;
p is an integer selected from 0, 1, or 2;
r is an integer selected from 0, 1, 2, or 3;
s is an integer selected from 1, 2, 3, 4, 5, 6, or 7;
t is an integer selected from 1 or 2;
u is an integer selected from 1 or 2;
v is an integer selected from 1, 2, or 3;
w is an integer selected from 1, 2, or 3;
   or an enantiomer, a stereoisomeric form, a mixture of enantiomers, a diastereomer, a mixture of diastereomers, a hydrate, a solvate, an acid salt form, a tautomer, a racemate of the above mentioned compounds, or a pharmaceutically acceptable salt thereof.

Thus, preferred are the compounds of formula (I),

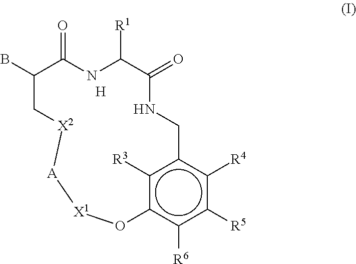

wherein
A represents —CO—N(R^{N6})—,

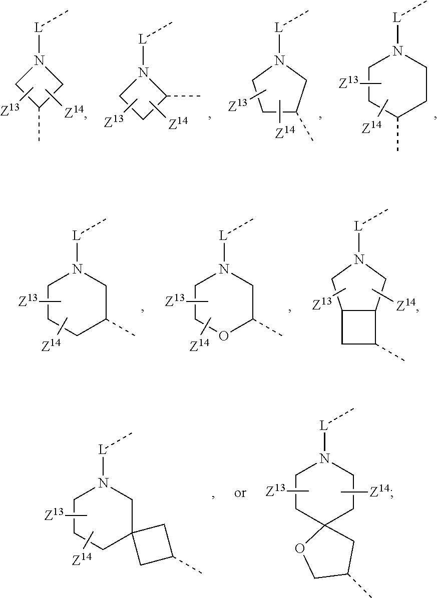

B represents —H, —NH(R²), —N(R²)(R^{N5}),

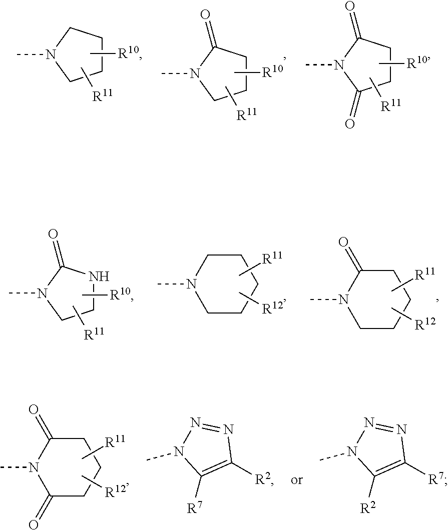

L represents —CO—, —CO—NH—, —CO—N(R^{N3})—, or —CO—O—;
R¹ represents —H, —(CH₂)ₚ—R⁷, —(CH₂)ₚ—NH—R⁷, —(CH₂)ₚ—R⁹, or —(CH₂)ₚ—NR^{N4}—R⁹;
R² represents —H, —R⁸, —R¹¹, -L¹-R¹¹, -L¹-(CH₂)ᵣ—R⁸, -L¹-R¹⁰, -L¹-(C₂H₄O)ₛ—R¹¹, -L¹-(CH₂)ₜ—O—R¹¹, -L¹-(CH₂)ₜ—NH—(CH₂)ᵣ—R⁸, -L¹-(CH₂)ₜ—O—(CH₂)ᵣ—R⁸, -L¹-(CH₂)ₜ—NHR⁸, -L¹-(CH₂)ₜ—NH—CO—R⁸, -L¹-(CH₂)ₜ—NH—SO₂—R⁸, -L¹-(CH₂)ₜ—NR^{N6}R¹⁰, -L¹-(CH₂)ₜ—O—(CH₂)ᵤ—NR^{N6}R¹⁰, -L¹-(CH₂)ᵣ—R¹⁴, —CO—C(R¹²)(R¹³)—R¹⁰, —CO—C(R¹²)(R¹³)—R⁸, or —CO—C(R¹²)(R¹³)—(CH₂)ᵤ—R⁸;
L¹ represents a bond, —CO—, —CO₂—, —CONH—, or —SO₂—;
R³-R⁶ represent independently of each other —H, —CH₃, —OCH₃, —F, or —Cl;
or R⁵ and R⁶ may form together

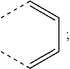

R⁸ and R⁹ represent independently of each other

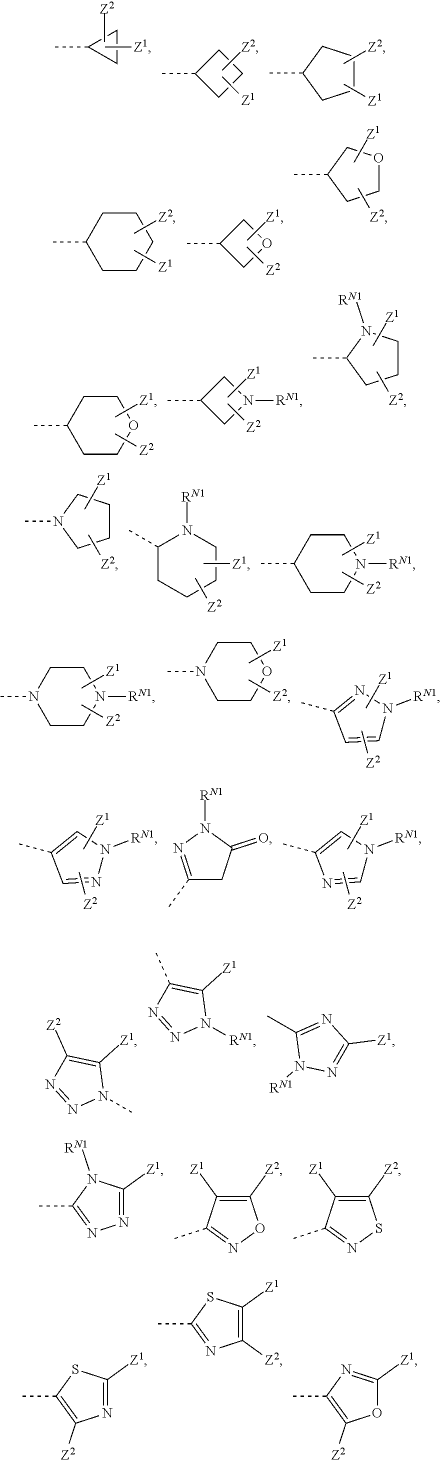

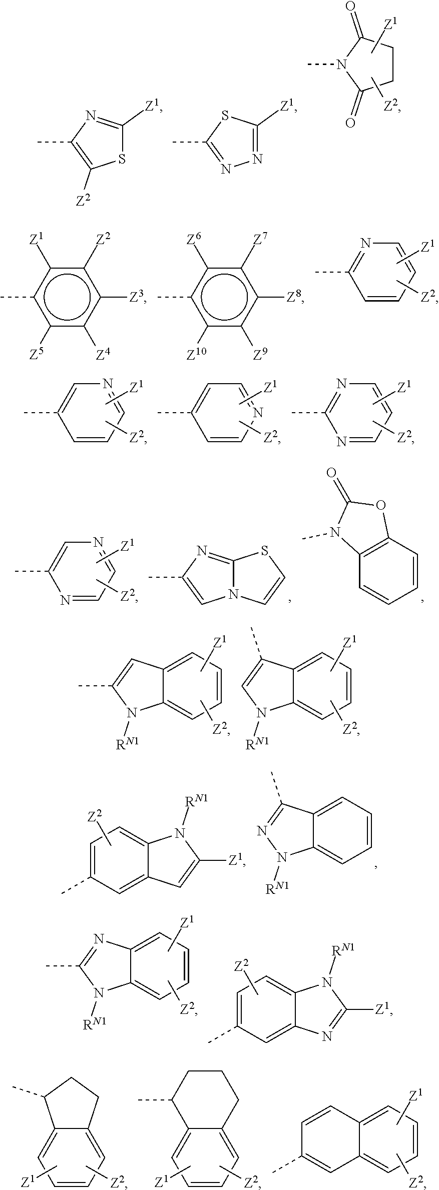

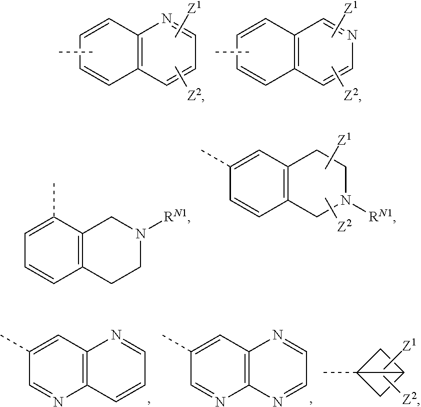

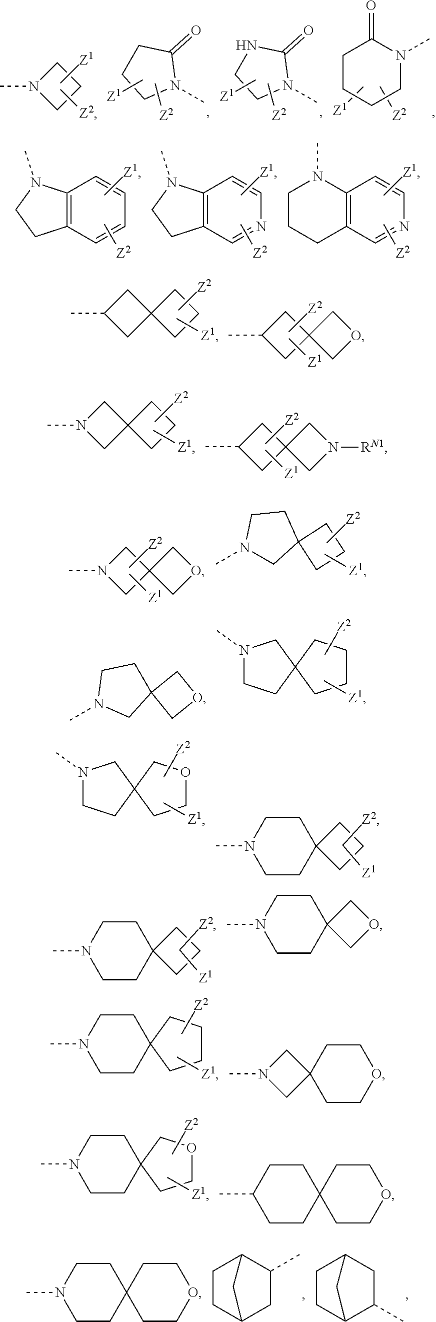

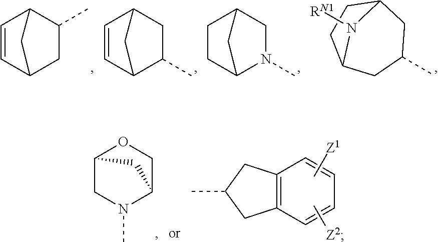

R⁷ and R¹⁰ represent independently of each other —H, —CH₃, —C₂H₅, —C₃H₇, —CH(CH₃)₂, —CH₂F, —CHF₂, —CF₃, —CH₂CN, —C(CH₃)₂—CN, —CH₂—C(CH₃)₂—CN, —CH₂—CF₃, —CH₂—C(CH₃)₂—NH₂,

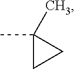

cyclo-C₃H₅, cyclo-C₄H₇, cyclo-C₅H₉, cyclo-C₆H₁₁, cyclo-C₇H₁₃, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, —C₇H₁₅, —C₈H₁₇, -Ph, —CH₂-Ph, —CH₂—CH₂-Ph, —C₂H₄—CH═CH₂, —CH₂—CH═CH—CH₃, —CH₂—C(CH₃)=CH₂, —CH(CH₃)—CH═CH₂, —C(CH₃)=CH—CH₃, —CH₂—CH═C(CH₃)₂, —CO—CH═C(CH₃)₂, —CH₂—C≡CH, —C₂H₄—C≡CH, —CH₂—C≡C—CH₃, —CH₂—OCF₃, —C₂H₄—OCF₃, —C₃H₆—OCF₃, —CH₂—OCHF₂, —C₂H₄—OCHF₂, —C₃H₆—OCHF₂, —CH₂—OCH₃, —C₂H₄—OCH₃, —C₃H₆—OCH₃, —CH₂—OC₂H₅, —C₂H₄—OC₂H₅, —C₃H₆—OC₂H₅, —CH₂—OH, —C₂H₄—OH, —C₃H₆—OH, —CH₂—COOH, —C₂H₄—COOH, —C₃H₆—OOH, —C(CH₃)₂—CN, —C(CH₃)₂—OH, —C(CH₃)₂—CH₂—OH, —C(C₂H₅)₂—CH₂—OH, —C(CH₂—OH)₂—CH₃, —C(CH₂—OH)₂—C₂H₅, —C(CH₃)₂—CH₂—SH, —C(C₂H₅)₂—CH₂—SH, —C(CH₂—SH)₂—CH₃, —CO—O—C(CH₃)₃, or —C(CH₂—SH)₂—C₂H₅;
   R¹¹ represents —H, —CH₃, —C₂H₅, —C₃H₇, —CH(CH₃)₂, —CH₂F, —CHF₂, —CF₃, —CH₂CN, —C(CH₃)₂—CN, —CH₂—C(CH₃)₂—CN, —CH₂—CF₃, —CH₂—C(CH₃)₂—NH₂,

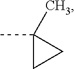

cyclo-C₃H₅, cyclo-C₄H₇, cyclo-C₅H₉, cyclo-C₆H₁₁, cyclo-C₇H₁₃, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, —C₇H₁₅, —C₈H₁₇, -Ph, —CH₂-Ph, —CH₂—CH₂-Ph, —CH═CH₂, —CH₂—CH═CH₂, —C(CH₃)=CH₂, —CH═CH—CH₃, —C(CH₃)=CH—CH₃, —CH═C(CH₃)₂, —C(CH₃)═C(CH₃)₂, —C₂H₄—CH═CH₂, —CH₂—CH═CH—CH₃, —CH₂—C(CH₃)=CH₂, —CH(CH₃)—CH═CH₂, —C(CH₃)=CH—CH₃, —CH₂—CH═C(CH₃)₂, —C≡CH, —CH₂—C≡CH, —C₂H₄—C≡CH, —CH₂—C≡C—CH₃, —C≡C—CH₃, —C≡C—C₂H₅, —CH₂—OCF₃, —C₂H₄—OCF₃, —C₃H₆—OCF₃, —CH₂—OCHF₂, —C₂H₄—OCHF₂, —C₃H₆—OCHF₂, —CH₂—OCH₃, —C₂H₄—OCH₃, —C₃H₆—OCH₃, —CH₂—OC₂H₅, —C₂H₄—OC₂H₅, —C₃H₆—OC₂H₅, —CH₂—OH, —C₂H₄—OH, —C₃H₆—OH, —C(CH₃)₂—CH₂—OH, —C(C₂H₅)₂—CH₂—OH, —C(CH₂—OH)₂—CH₃, —C(CH₂—OH)₂—C₂H₅, —C(CH₃)₂—CH₂—SH, —C(C₂H₅)₂—CH₂—SH, —C(CH₂—SH)₂—CH₃, or —C(CH₂—SH)₂—C₂H₅;
R¹² and R¹³ represent independently of each other —H, —CH₃, —C₂H₅, —C₃H₇, —CH(CH₃)₂, -Ph, —CH₂-Ph, —COOH, —NH₂, —NHCO₂(CCH₃)₃, —CH₂—NH₂, —CHF₂, —CF₃, —OCF₃, —OCHF₂, —OH, —F, —OCH₃, —OC₂H₅, —OC₃H₇, or —OCH(CH₃)₂; or
R¹² and R¹³ may form together

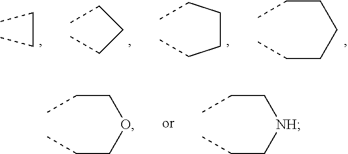

R¹⁴ represents

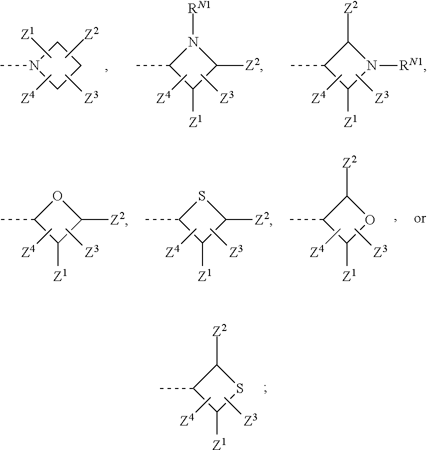

R¹⁵ and R¹⁶ represent independently of each other —X³-L²-R¹⁷, or —(OCH₂CH₂)_{w}—R¹⁷;
L² represents —(CH₂)ᵥ—, —(CH₂CH₂—O)_{w}—CH₂—, or —(CH₂CH₂—O)_{w}—CH₂CH₂—;
R¹⁷ represents —OH, —SH, —SO₃H, —NH₂, or —CO₂H;
R^{N1}, R^{N2}, R^{N3}, and R^{N4} represent independently of each other —R¹⁵, —H, —CH₃, —C₂H₅, —C₃H₇, —CH(CH₃)₂, —CHF₂, —CF₃,

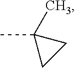

cyclo-C₃H₅, cyclo-C₄H₇, cyclo-C₅H₉, cyclo-C₆H₁₁, cyclo-C₇H₁₃, —C₄H₉, —CH₂—CH(CH₃)₂, —CH(CH₃)—C₂H₅, —C(CH₃)₃, —CH₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, —C₇H₁₅, —C₈H₁₇, -Ph, —CH₂-Ph, —CH₂—CH₂-Ph, —C₂H₄—CH═CH₂, —CH₂—CH═CH—CH₃, —CH₂—C(CH₃)=CH₂, —CH(CH₃)—CH═CH₂, —CH₂—CH═C(CH₃)₂, —CH₂—C≡CH, —C₂H₄—C≡CH, —CH₂—C≡C—CH₃, —COCH₃, —COC₂H₅, —COC₃H₇, —CO-cyclo-C₃H₅, —COCH(CH₃)₂, —COC(CH₃)₃, —COOH, —COOCH₃, —COOC₂H₅, —COOC₃H₇, —COO-cyclo-C₃H₅, —COOCH(CH₃)₂, —COOC(CH₃)₃, —COOCH₂Ph, —CONH₂, —CONHCH₃, —CONHC₂H₅, —CONHC₃H₇, —CONH-cyclo-C₃H₅, —CONH[CH(CH₃)₂], —CONH[C(CH₃)₃], —CON(CH₃)₂, —CON(C₂H₅)₂, —CON(C₃H₇)₂, —CON(cyclo-C₃H₅)₂, —CON[CH(CH₃)₂]₂, —CON[C(CH₃)₃]₂, or —SO₃H;
R^{N5} and R^{N6} represent independently of each other —H, —CH₃, —C₂H₅, —C₃H₇, —CH(CH₃)₂, cyclo-C₃H₅, —COOC(CH₃)₃, or —COOCH₂Ph;
X¹ represents —(CH₂)ₘ—;
X² represents —(CH₂)ₙ—;
X³ represents a bond, —O—, —NH—, or —S—;
Z¹-Z¹⁴ represent independently of each other

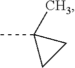

cyclo-C₃H₅, cyclo-C₄H₇, cyclo-C₅H₉, cyclo-C₆H₁₁, cyclo-C₇H₁₃, —R¹⁶, —H, —OH, —OCH₃, —OC₂H₅, —OC₃H₇, —O-cyclo-C₃H₅, —OCH(CH₃)₂, —OC(CH₃)₃, —OC₄H₉, —O-cyclo-C₄H₇, —O-cyclo-C₅H₉, —O-cyclo-C₆H₁₁, —OCH₂CH(CH₃)₂, —OCH₂-cyclo-C₃H₅, —OCH₂-cyclo-C₄H₇, —OCH₂-cyclo-C₅H₉, —OCH₂-cyclo-C₆H₁₁, —OPh, —OCH₂-Ph, —OCPh₃, —CH₂—OCH₃, —C₂H₄—OCH₃, —C₃H₆—OCH₃, —CH₂—OC₂H₅, —C₂H₄—OC₂H₅, —C₃H₆—OC₂H₅, —CH₂—OC₃H₇, —C₂H₄—OC₃H₇, —C₃H₆—OC₃H₇, —CH₂—O-cyclo-C₃H₅, —C₂H₄—O-cyclo-C₃H₅, —C₃H—O-cyclo-C₃H₅, —CH₂—OCH(CH₃)₂, —C₂H₄—OCH(CH₃)₂, —C₃H₆—OCH(CH₃)₂, —CH₂—OC(CH₃)₃, —C₂H₄—OC(CH₃)₃, —C₃H₆—OC(CH₃)₃, —CH₂—OC₄Hg, —C₂H₄—OC₄H₉, —C₃H₆—OC₄H₉, —CH₂—OPh, —C₂H₄—OPh, —C₃H₆—OPh, —CH₂—OCH₂-Ph, —C₂H₄—OCH₂-Ph, —C₃H₆—OCH₂-Ph, —SH, —SCH₃, —SC₂H₅, —SC₃H₇, —S-cyclo-C₃H₅, —SCH(CH₃)₂, —SC(CH₃)₃, —F, —Cl, —Br, —I, —CN, —COCH₃, —COC₂H₅, —COC₃H₇, —CO-cyclo-C₃H₅, —COCH(CH₃)₂, —COC(CH₃)₃, —COOH, —COOCH₃, —COOC₂H₅, —COOC₃H₇, —COO-cyclo-C₃H₅, —COOCH(CH₃)₂, —COOC(CH₃)₃, —OOC—CH₃, —OOC—C₂H₅, —OOC—C₃H₇, —OOC-cyclo-C₃H₅, —OOC—CH(CH₃)₂, —OOC—C(CH₃)₃, —CONH₂, —CONHCH₃, —CONHC₂H₅, —CONHC₃H₇, —CONH-cyclo-C₃H₅, —CONH[CH(CH₃)₂], —CONH[C(CH₃)₃], —CON(CH₃)₂, —CON(C₂H₅)₂, —CON(C₃H₇)₂, —CON(cyclo-C₃H₅)₂, —CON[CH(CH₃)₂]₂, —CON[C(CH₃)₃]₂, —NHCOCH₃, —NHCOC₂H₅, —NHCOC₃H₇, —NHCO-cyclo-C₃H₅, —NHCO—CH(CH₃)₂, —NHCO—C(CH₃)₃, —NHCO—CH(NH₂)CH₂—COOH, —NHCO—CH(NH₂)CH₂CH₂—COOH, —NHCO—OCH₃, —NHCO—OC₂H₅, —NHCO—OC₃H₇, —NHCO—O-cyclo-C₃H₅, —NHCO—OCH(CH₃)₂, —NHCO—OC(CH₃)₃, —NH₂, —NHCH₃, —NHC₂H₅, —NHC₃H₇, —NH-cyclo-C₃H₅, —NHCH(CH₃)₂, —NHC(CH₃)₃, —N(CH₃)₂, —N(C₂H₅)₂, —N(C₃H₇)₂, —N(cyclo-C₃H₅)₂, —N[CH(CH₃)₂]₂, —N[C(CH₃)₃]₂, —SOCH₃, —SOC₂H₅, —SOC₃H₇, —SO-cyclo-C₃H₅, —SOCH(CH₃)₂, —SOC(CH₃)₃, —SO₂CH₃, —SO₂C₂H₅, —SO₂C₃H₇, —SO₂-cyclo-C₃H₅, —SO₂CH(CH₃)₂, —SO₂C(CH₃)₃, —SO₃H, —SO₃CH₃, —SO₃C₂H₅, —SO₃C₃H₇, —SO₃-cyclo-C₃H₅, —SO₃CH(CH₃)₂, —SO₃C(CH₃)₃, —SO₂NH₂, —SO₂NHCH₃, —SO₂NHC₂H₅, —SO₂NHC₃H₇, —SO₂NH-cyclo-C₃H₅, —SO₂NHCH(CH₃)₂, —SO₂NHC(CH₃)₃, —SO₂N(CH₃)₂, —SO₂N(C₂H₅)₂, —SO₂N(C₃H₇)₂, —SO₂N(cyclo-C₃H₅)₂, —SO₂N[CH(CH₃)₂]₂, —SO₂N[C(CH₃)₃]₂, —O—S(═O)CH₃, —O—S(═O)C₂H₅, —O—S(═O)C₃H₇, —O—S(═O)-cyclo-C₃H₅, —O—S(═O)CH(CH₃)₂, —O—S(═O)C(CH₃)₃, —S(═O)(═NH)CH₃, —S(═O)(═NH)C₂H₅, —S(═O)(═NH)C₃H₇, —S(═O)(═NH)-cyclo-C₃H₅, —S(═O)(═NH)CH(CH₃)₂, —S(═O)(═NH)C(CH₃)₃, —NH—SO₂—CH₃, —NH—SO₂—C₂H₅, —NH—SO₂—C₃H₇, —NH—SO₂-cyclo-C₃H₅, —NH—SO₂—CH(CH₃)₂, —NH—SO₂—C(CH₃)₃, —O—SO₂—CH₃, —O—SO₂—C₂H₅, —O—SO₂—C₃H₇, —O—SO₂-cyclo-C₃H₅, —O—SO₂—CH(CH₃)₂, —O—SO₂—C(CH₃)₃, —OCH₂F, —OCHF₂, —OCF₃, —CH₂—OCF₃, —C₂H₄—OCF₃, —C₃H₆—OCF₃, —CH₂—OCHF₂, —C₂H₄—OCHF₂, —C₃H₆—OCHF₂, —OC₂F₅, —CH₂—OC₂F₅, —C₂H₄—OC₂F₅, —C₃H₆—OC₂F₅, —O—COOCH₃, —O—COOC₂H₅, —O—COOC₃H₇, —O—COO-cyclo-C₃H₅, —O—COOCH(CH₃)₂, —O—COOC(CH₃)₃, —NH—CO—NH₂, —NH—CO—NHCH₃, —NH—CO—NHC₂H₅, —NH—CO—NHC₃H₇, —NH—C(═NH)—NH₂, —NH—CO—N(C₃H₇)₂, —NH—CO—NH[CH(CH₃)₂], —NH—CO—NH[C(CH₃)₃], —NH—CO—N(CH₃)₂, —NH—CO—N(C₂H₅)₂, —NH—CO—NH-cyclo-C₃H₅, —NH—CO—N(cyclo-C₃H₅)₂, —NH—CO—N[CH(CH₃)₂]₂, —NH—C(═NH)—NHCH₃, —NH—C(═NH)—NHC₂H₅, —NH—C(═NH)—NHC₃H₇, —O—CO—NH-cyclo-C₃H₅, —NH—C(═NH)—NH-cyclo-C₃H₅, —NH—C(═NH)—NH[CH(CH₃)₂], —O—CO—NH[CH(CH₃)₂], —NH—C(═NH)—NH[C(CH₃)₃], —NH—C(═NH)—N(CH₃)₂, —NH—C(═NH)—N(C₂H₅)₂, —NH—C(═NH)—N(C₃H₇)₂, —NH—C(═NH)—N(cyclo-C₃H₅)₂, —O—CO—NHC₃H₇, —NH—C(═NH)—N[CH(CH₃)₂]₂, —NH—C(═NH)—N[C(CH₃)₃]₂, —O—CO—NH₂, —O—CO—NHCH₃, —O—CO—NHC₂H₅, —O—CO—NH[C(CH₃)₃], —O—CO—N(CH₃)₂, —O—CO—N(C₂H₅)₂, —O—CO—N(C₃H₇)₂, —O—CO—N(cyclo-C₃H₅)₂, —O—CO—N[CH(CH₃)₂]₂, —O—CO—N[C(CH₃)₃]₂, —O—CO—OCH₃, —O—CO—OC₂H₅, —O—CO—OC₃H₇, —CO—O—O-cyclo-C₃H₅, —O—CO—OCH(CH₃)₂, —O—CO—OC(CH₃)₃, —CH₂F, —CHF₂, —CF₃, —CH₂—CH₂F, —CH₂—CHF₂, —CH₂—CF₃, cyclo-C₈H₁₅, -Ph, —CH₂-Ph, —CH₂—CH₂-Ph, —CH═CH-Ph, —CPh₃, —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, —C₇H₁₅, —C₈H₁₇, —C₃H₆—CH(CH₃)₂, —C₂H₄—CH(CH₃)—C₂H₅, —CH(CH₃)—C₄H₉, —CH₂—CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, —CH(CH₃)—C(CH₃)₃, —CH═CH₂, —CH₂—CH═CH₂, —C(CH₃)=CH₂, —CH═CH—CH₃, —C₂H₄—CH═CH₂, —CH₂—CH═CH—CH₃, —CH═CH—C₂H₅, —CH₂—C(CH₃)=CH₂, —CH(CH₃)—CH═CH, —CH═C(CH₃)₂, —C(CH₃)=CH—CH₃, —CH═CH—CH═CH₂, —C₃H₆—CH═CH₂, —C₂H₄—CH═CH—CH₃, —CH₂—CH═CH—C₂H₅, —CH═CH—C₃H₇, —CH═CH—CH═CH—CH₃, —C₂H₄—C(CH₃)=CH₂, —CH₂—CH(CH₃)—CH═CH₂, —CH(CH₃)—CH₂—CH═CH₂, —CH₂—CH═C(CH₃)₂, —CH₂—C(CH₃)=CH—CH₃, —CH(CH₃)—CH═CH—CH₃, —CH═CH—CH(CH₃)₂, —CH═C(CH₃)—C₂H₅, —C(CH₃)=CH—C₂H₅, —C(CH₃)═C(CH₃)₂, —C(CH₃)₂—CH═CH₂, —CH(CH₃)—C(CH₃)=CH₂, —C₄H₈—CH═CH₂, —C₃H₆—CH═CH—CH₃, —C₂H₄—CH═CH—C₂H₅, —CH₂—CH═CH—C₃H₇, —CH═CH—C₄H₉, —C₃H₆—C(CH₃)=CH₂, —C₂H₄—CH(CH₃)—CH═CH₂, —CH₂—CH(CH₃)—CH₂—CH═CH₂, —C₂H₄—CH═C(CH₃)₂, —CH(CH₃)—C₂H₄—CH═CH₂, —C₂H₄—C(CH₃)=CH—CH₃, —CH₂—CH(CH₃)—CH═CH—CH₃, —CH(CH₃)—CH₂—CH═CH—CH₃, —CH₂—CH═CH—CH(CH₃)₂, —CH₂—CH═C(CH₃)—C₂H₅, —CH₂—C(CH₃)=CH—C₂H₅, —CH(CH₃)—CH═CH—C₂H₅, —CH═CH—CH₂—CH(CH₃)₂, —CH═CH—CH(CH₃)—C₂H₅, —CH═C(CH₃)—C₃H₇, —C(CH₃)=CH—C₃H₇, —CH₂—CH(CH₃)—C(CH₃)=CH₂, —C[C(CH₃)₃]=CH₂, —CH(CH₃)—CH₂—C(CH₃)=CH₂, —CH(CH₃)—CH(CH₃)—CH═CH₂, —CH═CH—C₂H₄—CH═CH₂, —C(CH₃)₂—CH₂—CH═CH₂, —CH₂—C(CH₃)═C(CH₃)₂, —CH(CH₃)—CH═C(CH₃)₂, —C(CH₃)₂—CH═CH—CH₃, —CH═CH—CH₂—CH═CH—CH₃, —CH(CH₃)—C(CH₃)=CH—CH₃, —CH═C(CH₃)—CH(CH₃)₂, —C(CH₃)=CH—CH(CH₃)₂, —C(CH₃)═C(CH₃)—C₂H₅, —CH═CH—C(CH₃)₃, —C(CH₃)₂—C(CH₃)=CH₂, —CH(C₂H₅)—C(CH₃)=CH₂, —C(CH₃)(C₂H₅)—CH═CH₂, —CH(CH₃)—C(C₂H₅)=CH₂, —CH₂—C(C₃H₇)=CH₂, —CH₂—C(C₂H₅)=CH—CH₃, —CH(C₂H₅)—CH═CH—CH₃, —C(C₄H₉)=CH₂, —C(C₃H₇)=CH—CH₃, —C(C₂H₅)=CH—C₂H₅, —C(C₂H₅)═C(CH₃)₂, —C[CH(CH₃)(C₂H₅)]=CH₂, —C[CH₂—CH(CH₃)₂]=CH₂, —C₂H₄—CH═CH—CH═CH₂, —CH₂—CH═CH—CH₂—CH═CH₂, —C₃H₆—C≡C—CH₃, —CH₂—CH═CH—CH═CH—CH₃, —CH═CH—CH═CH—C₂H₅, —CH(CH₃)—CH₂—C≡CH, —CH(CH₃)—C≡C—CH₃, —C₂H₄—CH(CH₃)—C≡CH, —CH═CH—CH═C(CH₃)₂, —CH₂—CH(CH₃)—CH₂—C≡CH, —CH═CH—C(CH₃)=CH—CH₃, —CH═C(CH₃)—CH═CH—CH₃, —CH₂—CH(CH₃)—C≡CH, —C(CH₃)=CH—CH═CH—CH₃, —CCH, —C≡C—CH₃, —CH₂—C≡CH, —C₂H₄—C≡CH, —CH₂—C≡C—CH₃, —C≡C—C₂H₅, —C₃H₆—C≡CH, —C₂H₄—C≡C—CH₃, —CH₂—C≡C—C₂H₅, —C≡C—C₃H₇, —CH(CH₃)—C≡CH, —C₄H₈—C≡CH, —C₂H₄—C≡C—C₂H₅, —CH₂—C≡C—C₃H₇, —C≡C—C₄H₉, —C≡C—CH₂—CH(CH₃)₂, —CH(CH₃)—C₂H₄—C≡CH, —CH₂—CH(CH₃)—C≡C—CH₃, —C(CH₃)(C₂H₅)—C≡CH, —CH(CH₃)—CH₂—C≡C—CH₃, —CH(CH₃)—C≡C—C₂H₅, —CH₂—C≡C—CH(CH₃)₂, —C≡C—CH(CH₃)—C₂H₅, —CH₂—C≡C—C≡C—CH₃, —CH(C₂H₅)—C≡C—CH₃, —C(CH₃)₂—C≡C—CH₃, —CH(C₂H₅)—CH₂—C≡CH, —CH₂—CH(C₂H₅)—C≡CH, —C(CH₃)₂—CH₂—C≡CH, —CH₂—C(CH₃)₂—C≡CH, —CH(CH₃)—CH(CH₃)—C≡CH, —CH(C₃H₇)—C≡CH, —CH₂—CH(C≡CH)₂, —C≡C—C≡CH, —CH₂—C≡C—C≡CH, —C≡C—C≡C—CH₃, —CH(C≡CH)₂, —C₂H₄—C≡C—C≡CH, —CH₂—C≡C—CH₂—C≡CH, —C≡C—C₂H₄—C≡CH, —C≡C—C(CH₃)₃, —C≡C—CH₂—C≡C—CH₃, —C≡C—C≡C—C₂H₅,

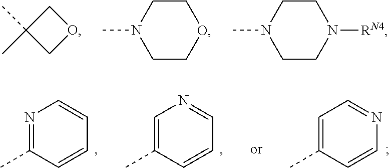

Z³ and Z⁴ may form together

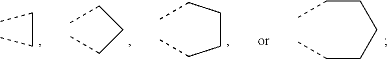

Z¹³ and Z¹⁴ may or form together

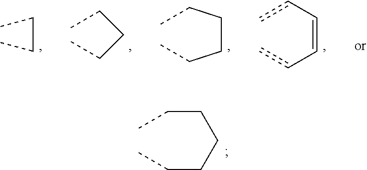

m is an integer selected from 0, 1, 2, 3, 4, 5, or 6;
n is an integer selected from 0, 1, 2, 3, 4, 5, or 6;
p is an integer selected from 0, 1, 2, 3, 4, 5, or 6;
r is an integer selected from 0, 1, 2, 3, or 4;
s is an integer selected from 1, 2, 3, 4, 5, 6, 7, 8, 9, or 10;
t is an integer selected from 1, 2, 3, or 4;
u is an integer selected from 1, 2, 3, or 4;
v is an integer selected from 0, 1, 2, 3, 4, 5, or 6;
w is an integer selected from 0, 1, 2, 3, 4, 5 or 6;
or an enantiomer, a stereoisomeric form, a mixture of enantiomers, a diastereomer, a mixture of diastereomers, a hydrate, a solvate, an acid salt form, a tautomer, a racemate of the above mentioned compounds, or a pharmaceutically acceptable salt thereof.

Still more preferred is the compound of formula (I), wherein R^{a} represents

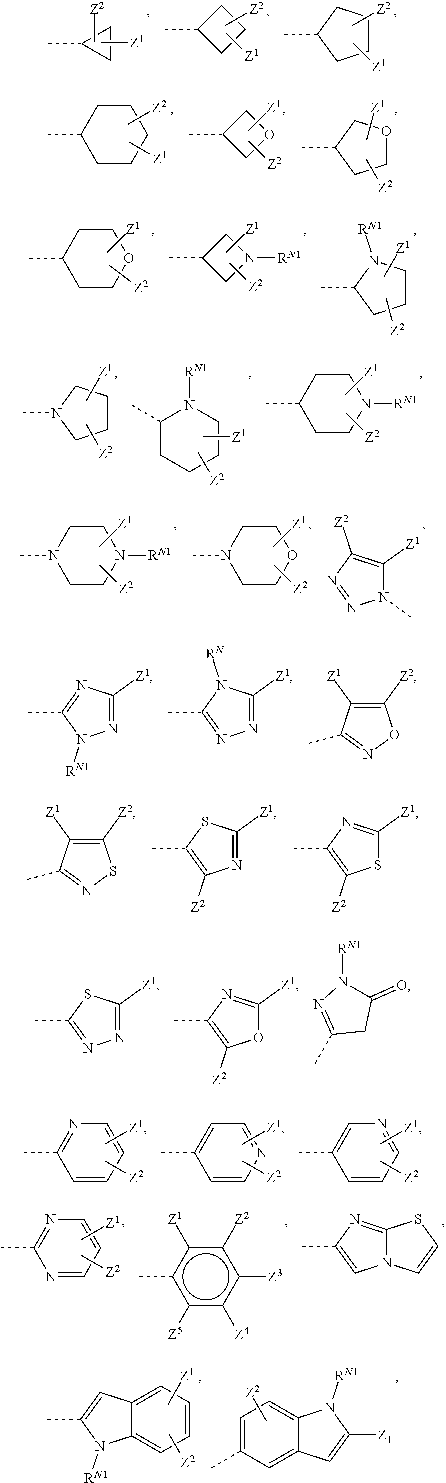

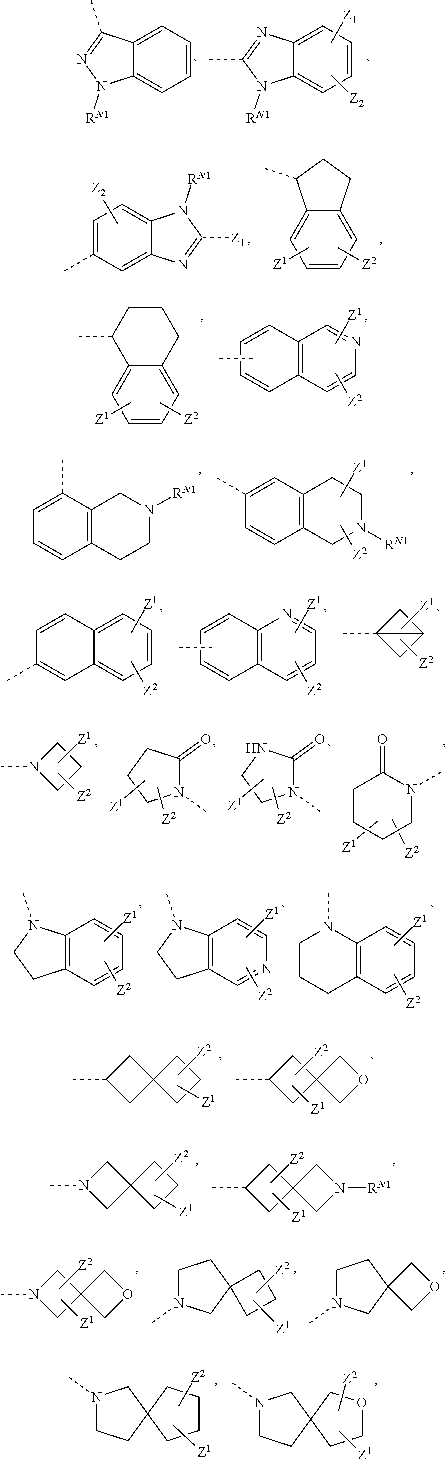

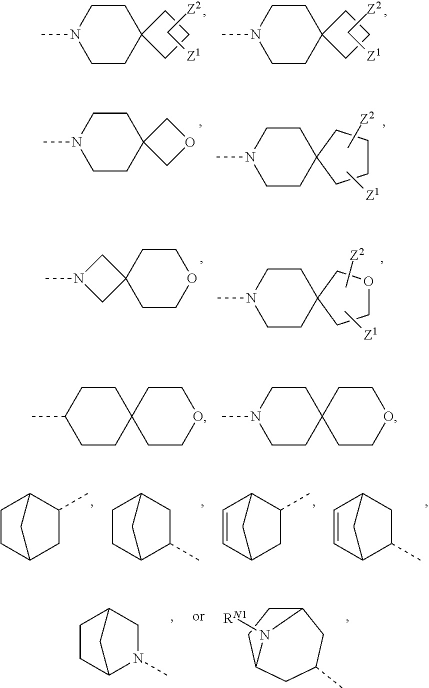

wherein R^{N1}, Z¹, Z², Z³, Z⁴, and Z⁵ have the same meanings as defined in formula (I).

Still preferred is the compound of the formula (I), wherein R⁹ represents

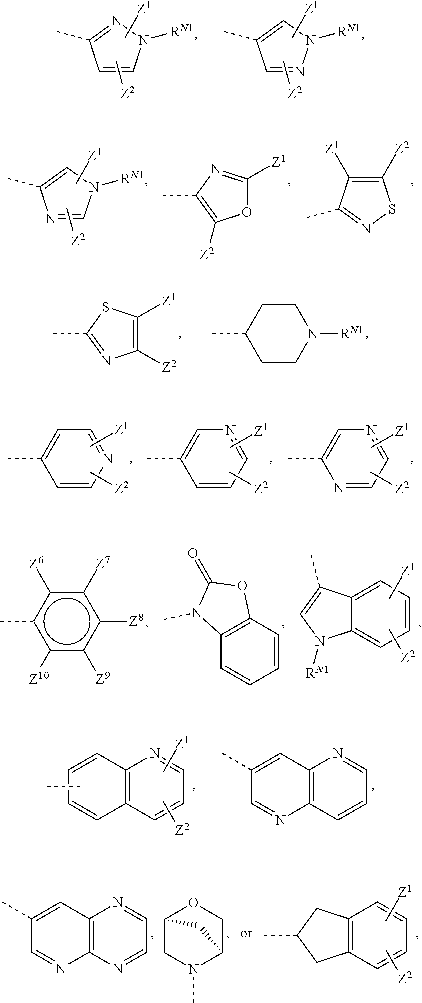

wherein R^{N1}, Z¹, Z², Z⁶, Z⁷, Z⁸, Z⁹, and Z¹⁰ have the same meanings as defined in formula (I).

Thus, preferred are the compounds of formula (I),

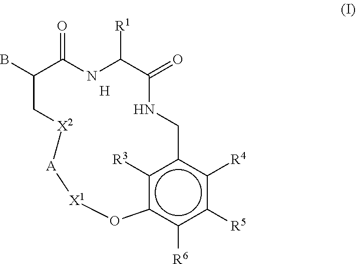

wherein
A represents —CO—N(R^{N6})—,

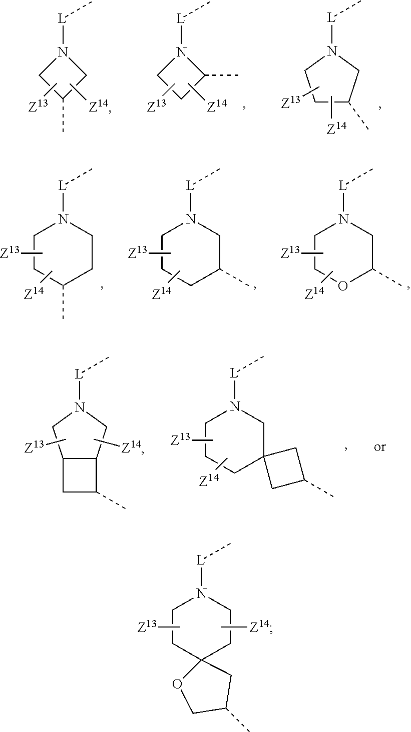

B represents —H, —NH(R²), —N(R²)(R^{N5}),

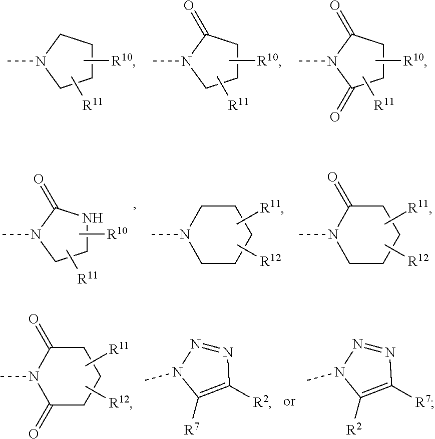

preferably,
B represents —H, —NH(R²), —N(R²)(R^{N5}),

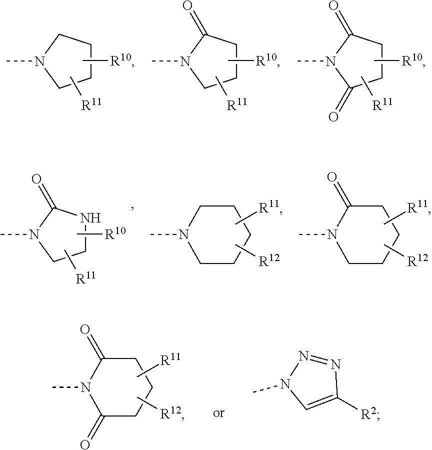

L represents —CO—, —CO—NH—, —CO—N(R^{N3})—, or —CO—O—;
R¹ represents —H, —(CH₂)ₚ—R⁷, —(CH₂)ₚ—NH—R⁷, —(CH₂)ₚ—R⁹, or —(CH₂)ₚ—NR^{N4}—R⁹;
R² represents —H, —R⁸, —R¹¹, -L¹-R¹¹, -L¹-(CH₂)ᵣ—R⁸, -L¹-R¹⁰, -L¹-(C₂H₄O)ₛ—R¹¹, -L¹-(CH₂)ₜ—O—R¹¹, -L¹-(CH₂)ₜ—NH—(CH₂)ᵣ—R⁸, -L¹-(CH₂)ₜ—O—(CH₂)ᵣ—R⁸, -L¹-(CH₂)ₜ—NHR⁸, -L¹-(CH₂)ₜ—NH—CO—R⁸, -L¹-(CH₂)ₜ—NH—SO₂—R⁸, -L¹-(CH₂)ₜ—NR^{N6}R¹⁰, -L¹-(CH₂)ₜ—O—(CH₂)ᵤ—NR^{N6}R¹⁰, -L¹-(CH₂)ᵣ—R¹⁴, —CO—C(R¹²)(R¹³)—R¹⁰, —CO—C(R¹²)(R¹³)—R⁸, or —CO—C(R¹²)(R¹³)—(CH₂)ᵤ—R⁸;
L¹ represents a bond, —CO—, —CO₂—, —CONH—, or —SO₂—;
R³-R⁶ represent independently of each other —H, —CH₃, —OCH₃, —F, or —Cl;
or R 5 and R⁶ may form together

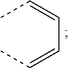

R⁸ represents

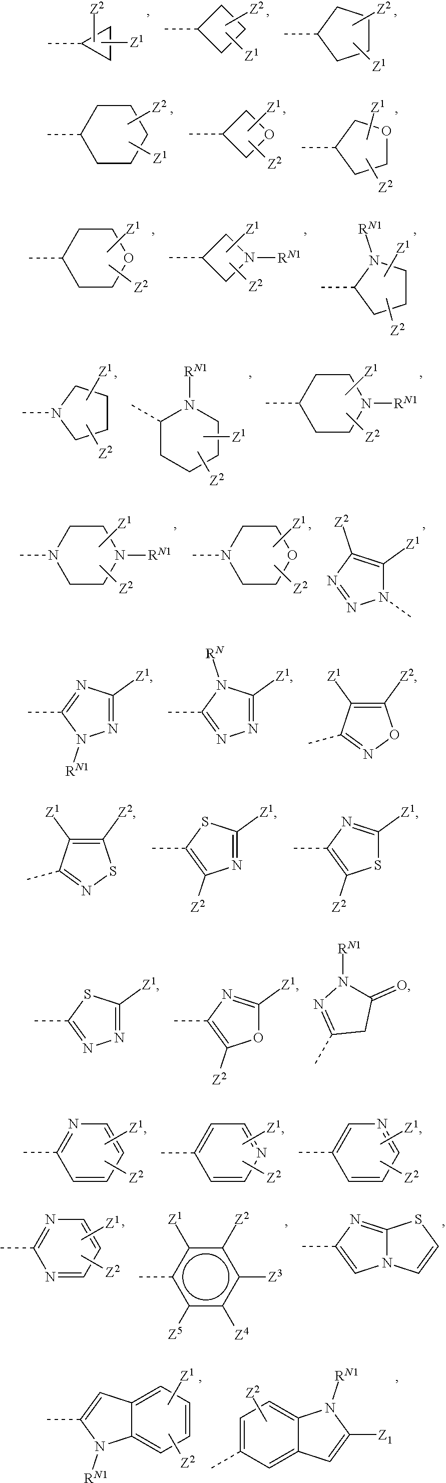

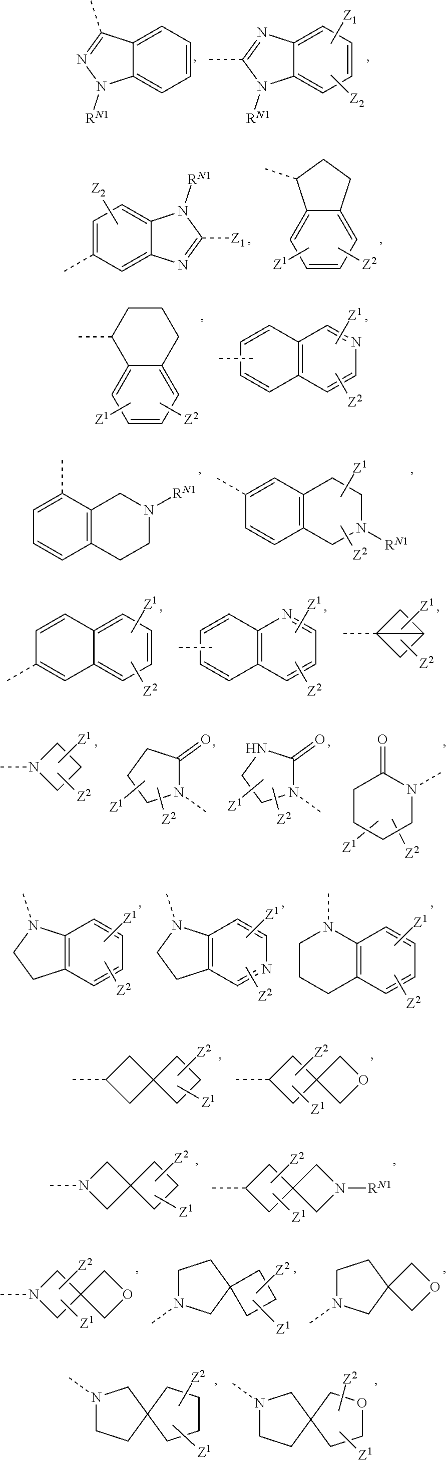

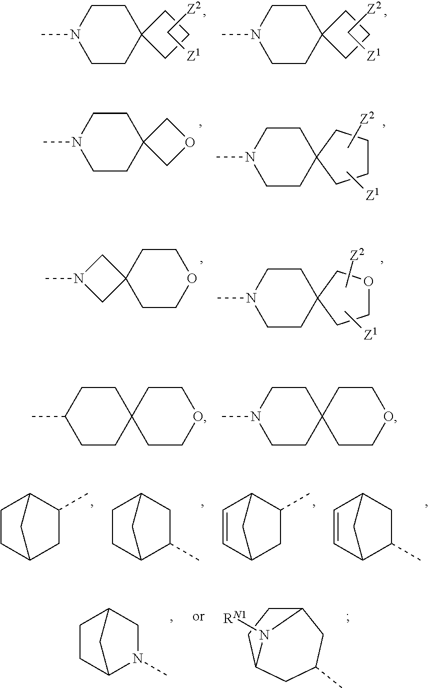

R⁹ represents

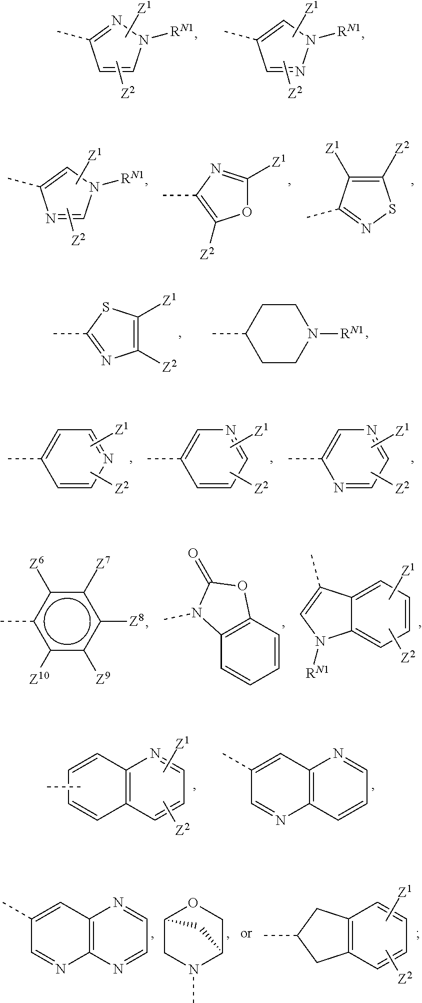

X¹ represents —(CH₂)ₘ—;
X² represents —(CH₂)ₙ—;
X³ represents a bond, —O—, —NH—, or —S—;
and R^{N1}—R^{N6}, R⁷, R¹⁰-R¹⁷, Z¹-Z¹⁴, m, n, o, p, r, s, t, u, v, w have the same meanings as defined above,
or an enantiomer, a stereoisomeric form, a mixture of enantiomers, a diastereomer, a mixture of diastereomers, a hydrate, a solvate, an acid salt form, a tautomer, a racemate of the above mentioned compounds, or a pharmaceutically acceptable salt thereof.

Preferably, R¹⁵ represents
—CH₂—OH, —CH₂CH₂—OH, —CH₂CH₂CH₂—OH, —CH₂CH₂CH₂CH₂—OH, —CH₂CH₂CH₂CH₂CH₂—OH, —CH₂CH₂CH₂CH₂CH₂—OH, —CH₂—CO₂H, —CH₂CH₂—CO₂H, —CH₂CH₂CH₂—CO₂H, —CH₂CH₂CH₂CH₂—CO₂H, —CH₂CH₂CH₂CH₂CH₂—CO₂H, —CH₂CH₂CH₂CH₂CH₂—CO₂H, —CH₂—SH, —CH₂CH₂—SH, —CH₂CH₂CH₂—SH, —CH₂CH₂CH₂CH₂—SH, —CH₂CH₂CH₂CH₂CH₂—SH, —CH₂CH₂CH₂CH₂CH₂—SH, —CH₂—SO₃H, —CH₂CH₂—SO₃H, —CH₂CH₂CH₂—SO₃H, —CH₂CH₂CH₂CH₂—SO₃H, —CH₂CH₂CH₂CH₂CH₂—SO₃H, —CH₂CH₂CH₂CH₂CH₂—SO₃H, —CH₂—NH₂, —CH₂CH₂—NH₂, —CH₂CH₂CH₂—NH₂, —CH₂CH₂CH₂CH₂—NH₂, —CH₂CH₂CH₂CH₂CH₂—NH₂, —CH₂CH₂CH₂CH₂CH₂CH₂—NH₂, —CH₂CH₂—O—CH₂—CH₂—OH, —(CH₂CH₂—O)₂—CH₂—CH₂—OH, —(CH₂CH₂—O)₃—CH₂—CH₂—OH, —CH₂CH₂—O—CH₂—CH₂—CO₂H, —(CH₂CH₂—O)₂—CH₂—CH₂—CO₂H, —(CH₂CH₂—O)₃—CH₂—CH₂—CO₂H, —CH₂CH₂—O—CH₂—CH₂—SH, —(CH₂CH₂—O)₂—CH₂—CH₂—SH, —(CH₂CH₂—O)₃—CH₂—CH₂—SH, —CH₂CH₂—O—CH₂—CH₂—SO₃H, —(CH₂CH₂—O)₂—CH₂—CH₂—SO₃H, —(CH₂CH₂—O)₃—CH₂—CH₂—SO₃H, —CH₂CH₂—O—CH₂—CH₂—NH₂, —(CH₂CH₂—O)₂—CH₂—CH₂—NH₂, or —(CH₂CH₂—O)₃—CH₂—CH₂—NH₂.

More preferably, R¹⁵ represents —CH₂—NH₂, —CH₂CH₂—NH₂, —CH₂CH₂CH₂—NH₂, —CH₂—SO₃H, —CH₂CH₂—SO₃H, —CH₂CH₂CH₂—SO₃H, —CH₂CH₂—O—CH₂—CH₂—NH₂, —(CH₂CH₂—O)₂—CH₂—CH₂—NH₂, or —(CH₂CH₂—O)₃—CH₂—CH₂—NH₂.

Preferably, R¹⁶ represents
—CH₂—OH, —CH₂CH₂—OH, —CH₂CH₂CH₂—OH, —CH₂CH₂CH₂CH₂—OH, —CH₂CH₂CH₂CH₂CH₂—OH, —CH₂CH₂CH₂CH₂CH₂—OH, —CH₂—CO₂H, —CH₂CH₂—CO₂H, —CH₂CH₂CH₂—CO₂H, —CH₂CH₂CH₂CH₂—CO₂H, —CH₂CH₂CH₂CH₂CH₂—CO₂H, —CH₂CH₂CH₂CH₂CH₂—CO₂H, —CH₂—SH, —CH₂CH₂—SH, —CH₂CH₂CH₂—SH, —CH₂CH₂CH₂CH₂—SH, —CH₂CH₂CH₂CH₂CH₂—SH, —CH₂CH₂CH₂CH₂CH₂—SH, —CH₂—SO₃H, —CH₂CH₂—SO₃H, —CH₂CH₂CH₂—SO₃H, —CH₂CH₂CH₂CH₂—SO₃H, —CH₂CH₂CH₂CH₂CH₂—SO₃H, —CH₂CH₂CH₂CH₂CH₂—SO₃H, —CH₂—NH₂, —CH₂CH₂—NH₂, —CH₂CH₂CH₂—NH₂, —CH₂CH₂CH₂CH₂—NH₂, —CH₂CH₂CH₂CH₂CH₂—NH₂, —CH₂CH₂CH₂CH₂CH₂CH₂—NH₂, —CH₂CH₂—O—CH₂—CH₂—OH, —(CH₂CH₂—O)₂—CH₂—CH₂—OH, —(CH₂CH₂—O)₃—CH₂—CH₂—OH, —CH₂CH₂—O—CH₂—CH₂—CO₂H, —(CH₂CH₂—O)₂—CH₂—CH₂—CO₂H, —(CH₂CH₂—O)₃—CH₂—CH₂—CO₂H, —CH₂CH₂—O—CH₂—CH₂—SH, —(CH₂CH₂—O)₂—CH₂—CH₂—SH, —(CH₂CH₂—O)₃—CH₂—CH₂—SH, —CH₂CH₂—O—CH₂—CH₂—SO₃H, —(CH₂CH₂—O)₂—CH₂—CH₂—SO₃H, —(CH₂CH₂—O)₃—CH₂—CH₂—SO₃H, —CH₂CH₂—O—CH₂—CH₂—NH₂, —(CH₂CH₂—O)₂—CH₂—CH₂—NH₂, —(CH₂CH₂—O)₃—CH₂—CH₂—NH₂, —OCH₂—OH, —OCH₂CH₂—OH, —OCH₂CH₂CH₂—OH, —OCH₂CH₂CH₂CH₂—OH, —OCH₂CH₂CH₂CH₂CH₂—OH, —OCH₂CH₂CH₂CH₂CH₂—OH, —OCH₂—CO₂H, —OCH₂CH₂—CO₂H, —OCH₂CH₂CH₂—CO₂H, —OCH₂CH₂CH₂CH₂—CO₂H, —OCH₂CH₂CH₂CH₂CH₂—CO₂H, —OCH₂CH₂CH₂CH₂CH₂—CO₂H, —OCH₂—SH, —OCH₂CH₂—SH, —OCH₂CH₂CH₂—SH, —OCH₂CH₂CH₂CH₂—SH, —OCH₂CH₂CH₂CH₂CH₂—SH, —OCH₂CH₂CH₂CH₂CH₂—SH, —OCH₂—SO₃H, —OCH₂CH₂—SO₃H, —OCH₂CH₂CH₂—SO₃H, —OCH₂CH₂CH₂CH₂—SO₃H, —OCH₂CH₂CH₂CH₂CH₂—SO₃H, —OCH₂CH₂CH₂CH₂CH₂—SO₃H, —OCH₂—NH₂, —OCH₂CH₂—NH₂, —OCH₂CH₂CH₂—NH₂, —OCH₂CH₂CH₂CH₂—NH₂, —OCH₂CH₂CH₂CH₂CH₂—NH₂, —OCH₂CH₂CH₂CH₂CH₂CH₂—NH₂, —OCH₂CH₂—O—CH₂—CH₂—OH, —O(CH₂CH₂—O)₂—CH₂—CH₂—OH, —O(CH₂CH₂—O)₃—CH₂—CH₂—OH, —OCH₂CH₂—O—CH₂—CH₂—CO₂H, —O(CH₂CH₂—O)₂—CH₂—CH₂—CO₂H, —O(CH₂CH₂—O)₃—CH₂—CH₂—CO₂H, —OCH₂CH₂—O—CH₂—CH₂—SH, —O(CH₂CH₂—O)₂—CH₂—CH₂—SH, —O(CH₂CH₂—O)₃—CH₂—CH₂—SH, —OCH₂CH₂—O—CH₂—CH₂—SO₃H, —O(CH₂CH₂—O)₂—CH₂—CH₂—SO₃H, —O(CH₂CH₂—O)₃—CH₂—CH₂—SO₃H, —OCH₂CH₂—O—CH₂—CH₂—NH₂, —O(CH₂CH₂—O)₂—CH₂—CH₂—NH₂, —O(CH₂CH₂—O)₃—CH₂—CH₂—NH₂, —SCH₂—OH, —SCH₂CH₂—OH, —SCH₂CH₂CH₂—OH, —SCH₂CH₂CH₂CH₂—OH, —SCH₂CH₂CH₂CH₂CH₂—OH, —SCH₂CH₂CH₂CH₂CH₂—OH, —SCH₂—CO₂H, —SCH₂CH₂—CO₂H, —SCH₂CH₂CH₂—CO₂H, —SCH₂CH₂CH₂CH₂—CO₂H, —SCH₂CH₂CH₂CH₂CH₂—CO₂H, —SCH₂CH₂CH₂CH₂CH₂—CO₂H, —SCH₂—SH, —SCH₂CH₂—SH, —SCH₂CH₂CH₂—SH, —SCH₂CH₂CH₂CH₂—SH, —SCH₂CH₂CH₂CH₂CH₂—SH, —SCH₂CH₂CH₂CH₂CH₂—SH, —SCH₂—SO₃H, —SCH₂CH₂—SO₃H, —SCH₂CH₂CH₂—SO₃H, —SCH₂CH₂CH₂CH₂—SO₃H, —SCH₂CH₂CH₂CH₂CH₂—SO₃H, —SCH₂CH₂CH₂CH₂CH₂—SO₃H, —SCH₂—NH₂, —SCH₂CH₂—NH₂, —SCH₂CH₂CH₂—NH₂, —SCH₂CH₂CH₂CH₂—NH₂, —SCH₂CH₂CH₂CH₂CH₂—NH₂, —SCH₂CH₂CH₂CH₂CH₂CH₂—NH₂, —SCH₂CH₂—O—CH₂—CH₂—OH, —S(CH₂CH₂—O)₂—CH₂—CH₂—OH, —S(CH₂CH₂—O)₃—CH₂—CH₂—OH, —SCH₂CH₂—O—CH₂—CH₂—CO₂H, —S(CH₂CH₂—O)₂—CH₂—CH₂—CO₂H, —S(CH₂CH₂—O)₃—CH₂—CH₂—CO₂H, —SCH₂CH₂—O—CH₂—CH₂—SH, —S(CH₂CH₂—O)₂—CH₂—CH₂—SH, —S(CH₂CH₂—O)₃—CH₂—CH₂—SH, —SCH₂CH₂—O—CH₂—CH₂—SO₃H, —S(CH₂CH₂—O)₂—CH₂—CH₂—SO₃H, —S(CH₂CH₂—O)₃—CH₂—CH₂—SO₃H, —SCH₂CH₂—O—CH₂—CH₂—NH₂, —S(CH₂CH₂—O)₂—CH₂—CH₂—NH₂, —S(CH₂CH₂—O)₃—CH₂—CH₂—NH₂, —NHCH₂—OH, —NHCH₂CH₂—OH, —NHCH₂CH₂CH₂—OH, —NHCH₂CH₂CH₂CH₂—OH, —NHCH₂CH₂CH₂CH₂CH₂—OH, —NHCH₂CH₂CH₂CH₂CH₂—OH, —NHCH₂—CO₂H, —NHCH₂CH₂—CO₂H, —NHCH₂CH₂CH₂—CO₂H, —NHCH₂CH₂CH₂CH₂—CO₂H, —NHCH₂CH₂CH₂CH₂CH₂—CO₂H, —NHCH₂CH₂CH₂CH₂CH₂—CO₂H, —NHCH₂—SH, —NHCH₂CH₂—SH, —NHCH₂CH₂CH₂—SH, —NHCH₂CH₂CH₂CH₂—SH, —NHCH₂CH₂CH₂CH₂CH₂—SH, —NHCH₂CH₂CH₂CH₂CH₂—SH, —NHCH₂—SO₃H, —NHCH₂CH₂—SO₃H, —NHCH₂CH₂CH₂—SO₃H, —NHCH₂CH₂CH₂CH₂—SO₃H, —NHCH₂CH₂CH₂CH₂CH₂—SO₃H, —NHCH₂CH₂CH₂CH₂CH₂—SO₃H, —NHCH₂—NH₂, —NHCH₂CH₂—NH₂, —NHCH₂CH₂CH₂—NH₂, —NHCH₂CH₂CH₂CH₂—NH₂, —NHCH₂CH₂CH₂CH₂CH₂—NH₂, —NHCH₂CH₂CH₂CH₂CH₂CH₂—NH₂, —NHCH₂CH₂—O—CH₂—CH₂—OH, —NH(CH₂CH₂—O)₂—CH₂—CH₂—OH, —NH(CH₂CH₂—O)₃—CH₂—CH₂—OH, —NHCH₂CH₂—O—CH₂—CH₂—CO₂H, —NH(CH₂CH₂—O)₂—CH₂—CH₂—CO₂H, —NH(CH₂CH₂—O)₃—CH₂—CH₂—CO₂H, —NHCH₂CH₂—O—CH₂—CH₂—SH, —NH(CH₂CH₂—O)₂—CH₂—CH₂—SH, —NH(CH₂CH₂—O)₃—CH₂—CH₂—SH, —NHCH₂CH₂—O—CH₂—CH₂—SO₃H, —NH(CH₂CH₂—O)₂—CH₂—CH₂—SO₃H, —NH(CH₂CH₂—O)₃—CH₂—CH₂—SO₃H, —NHCH₂CH₂—O—CH₂—CH₂—NH₂, —NH(CH₂CH₂—O)₂—CH₂—CH₂—NH₂, or —NH(CH₂CH₂—O)₃—CH₂—CH₂—NH₂.

More preferably, R¹⁶ represents —CH₂—NH₂, —CH₂CH₂—NH₂, —CH₂CH₂CH₂—NH₂, —CH₂—SO₃H, —CH₂CH₂—SO₃H, —CH₂CH₂CH₂—SO₃H, —CH₂CH₂—O—CH₂—CH₂—NH₂, —(CH₂CH₂—O)₂—CH₂—CH₂—NH₂, or —(CH₂CH₂—O)₃—CH₂—CH₂—NH₂.

Also preferred is the compound of formula (I), wherein A is —CO—NH—, —CO—NCH₃—,

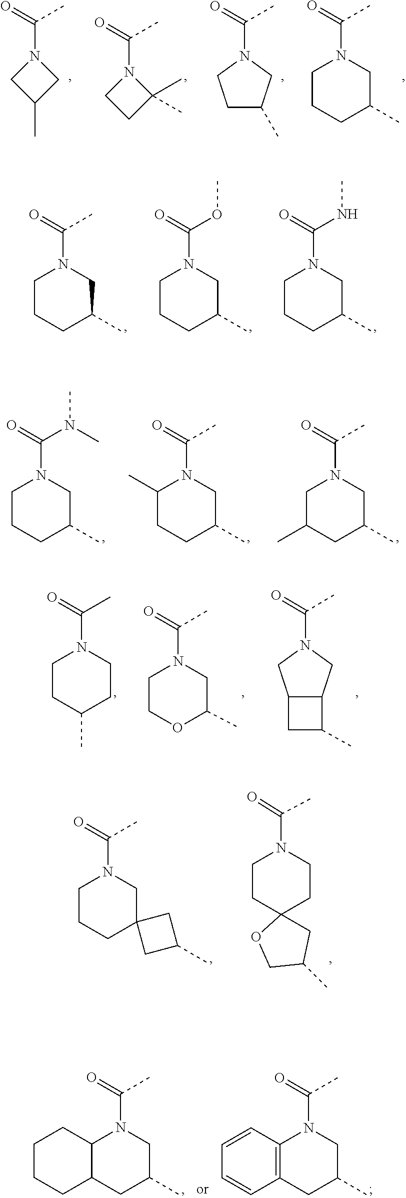

and/or
X₂ represents a bond, —CH₂—, —CH₂CH₂—, or —CH₂CH₂CH₂—; and
X₁ represents a bond, —CH₂—, —CH₂CH₂—, —CH₂CH₂CH₂—, or —CH₂CH₂CH₂CH₂—;
more preferred are compounds wherein —X₂-A-X₁— represents

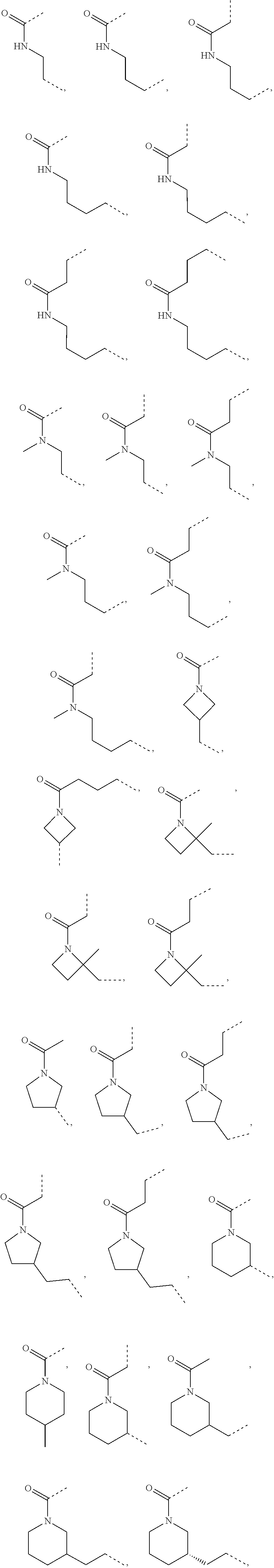

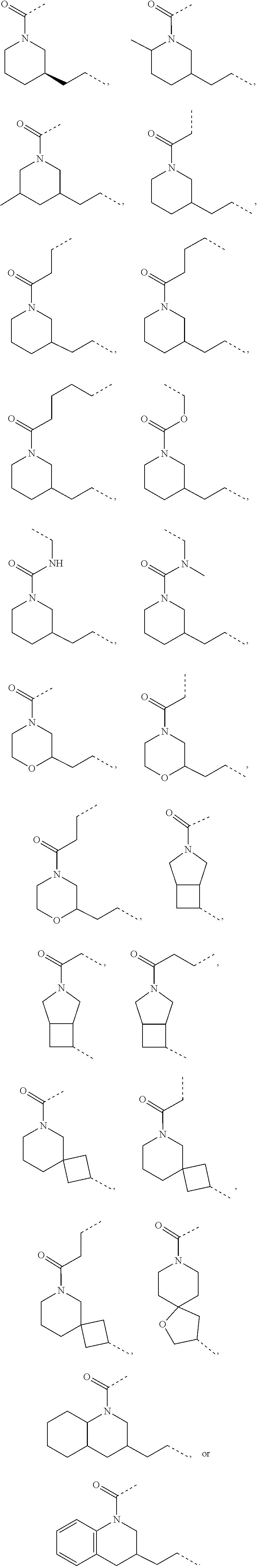

Also preferred is the compound of general formula (I), wherein
B represents —H, —NH₂, —NHCOCH₃, —NHCOC(CH₃)₃, —NHCOC(CN)(CH₃)₂, —NHCOCH═CH₂, —NHCOCH₂C(CH₃)₃, —NHCOPh, —NHCOCH₂Ph, —NHCOCH₂—NH(CH₃), —NHCOCH₂—N(CH₃)CO₂tBu, —NHCOC(CH₃)₂CH₂OH, —NHCOC(CH₃)₂CH₂SH, —NHCOC(CH₃)₂CH₂NH₂, —NHCOC(CH₃)₂CH₂F, —NHCOC(CH₃)₂CF₃, —NHCOCF₂CH₂OH, —NHCOCF₂CH₂NH₂, —NHCOC(CH₂CH₃)₂CH₂OH, —NHCOC(CH₃)(CH₂OH)₂, —NHCOCH₂OCH₂CH₂—NH(CH₃), —NHCOCH₂OCH₂CH₂—N(CH₃)CO₂tBu, —NHCO(CH₂CH₂O)₂CH₃, —NHCO₂CH₂CH₃, —NHCO₂(CH₂CH₂O)₃CH₃, —NHCO₂(CH₂CH₂O)₅CH₃, —NHCO₂(CH₂CH₂O)₇CH₃, —NHCO₂CH₂Ph —NHCONHCH₂CH₃, —NHSO₂CH₃, —NHSO₂CH═CH₂, —NHSO₂CH₂Ph, —NHSO₂CH₂CH₂Ph, —NHSO₂CH₂CH₂CH₂Ph, —NHSO₂CH₂CF₃, —NHCH₂CF₃, —NHCH₂(CH₃)₂NH₂, —NHCH₂(CH₃)₂CH₂OH,

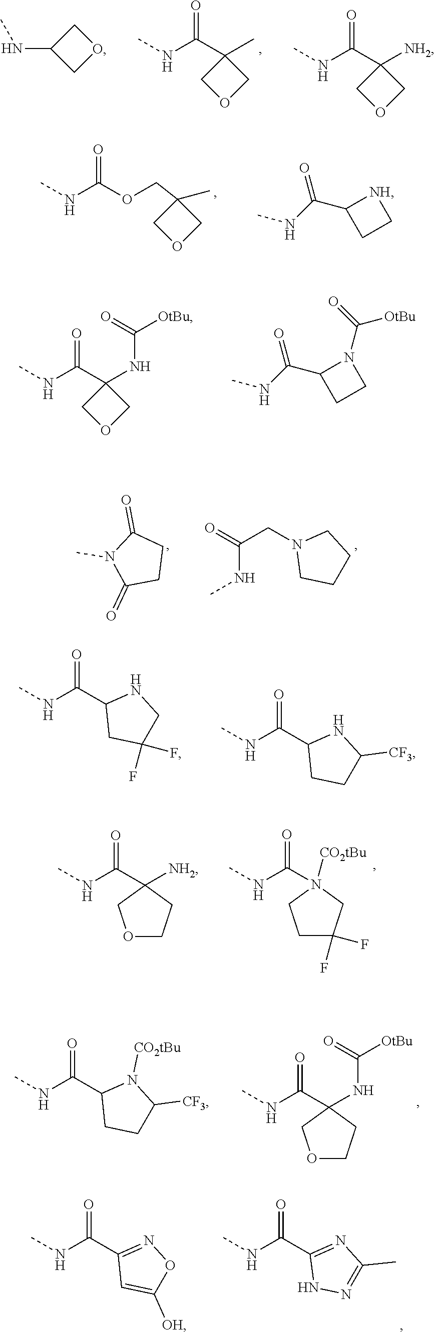

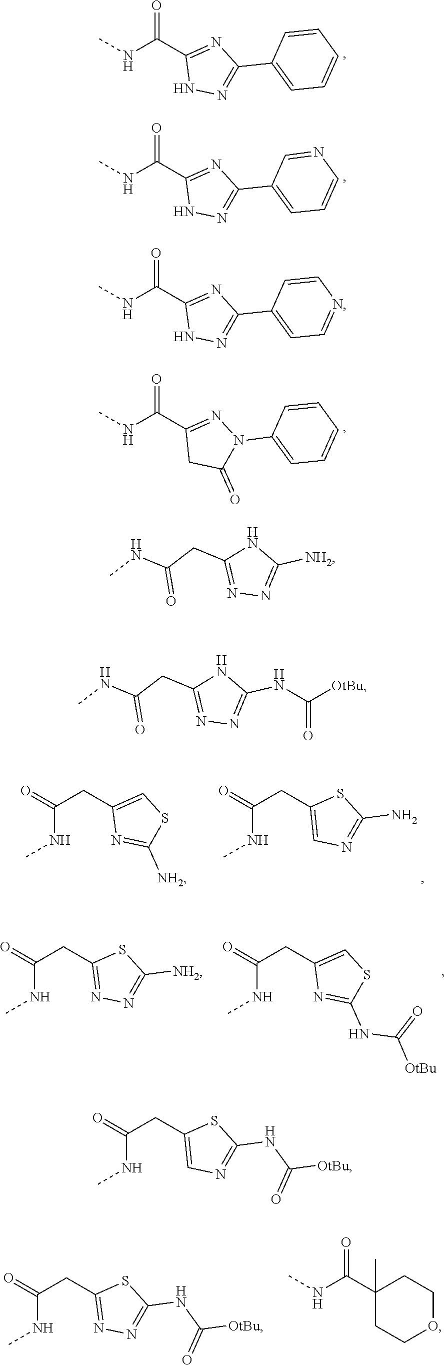

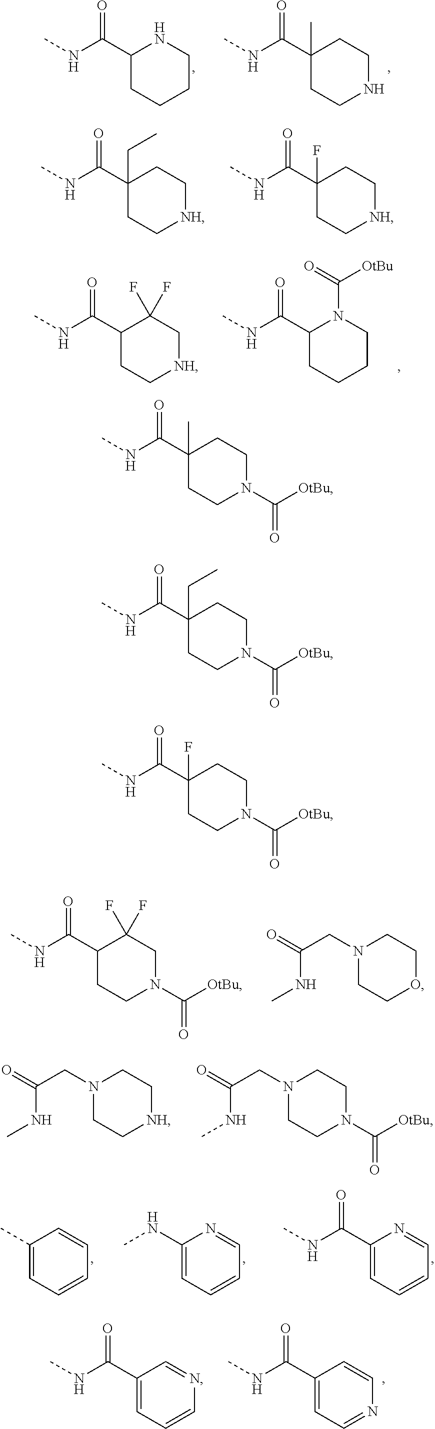

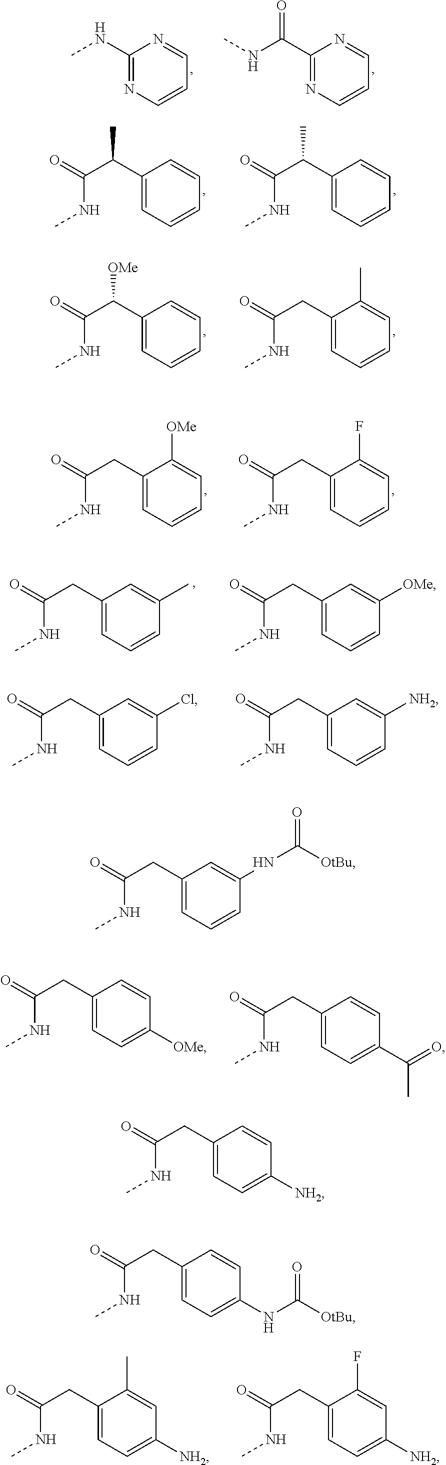

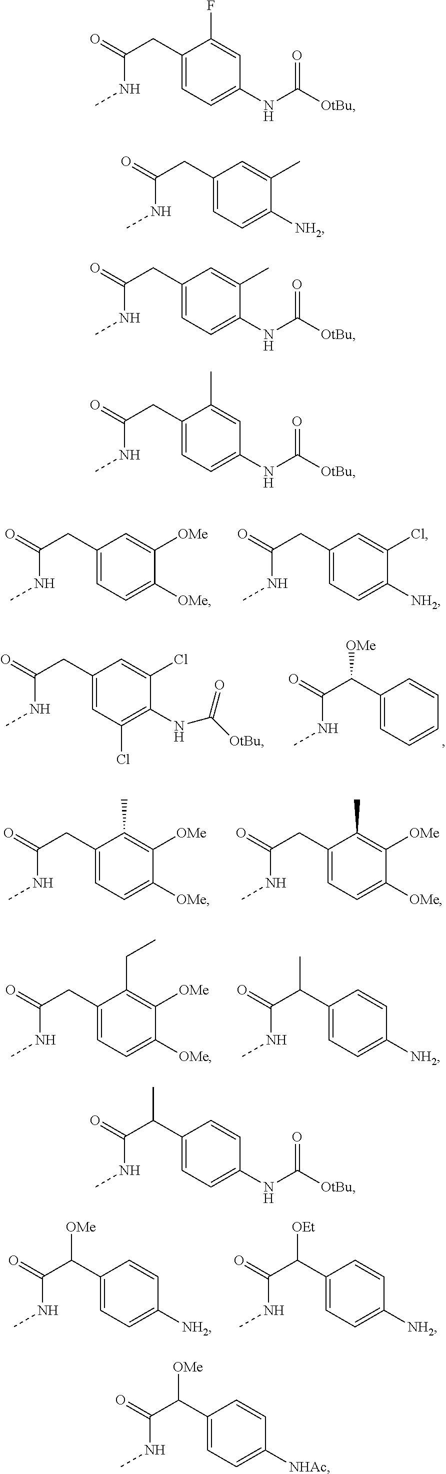

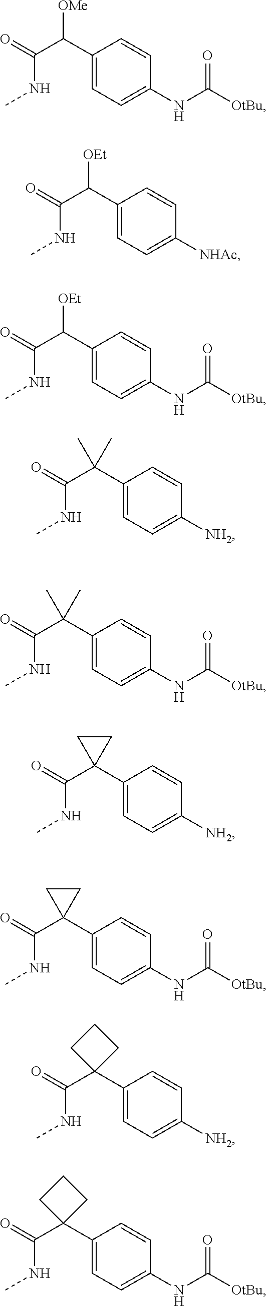

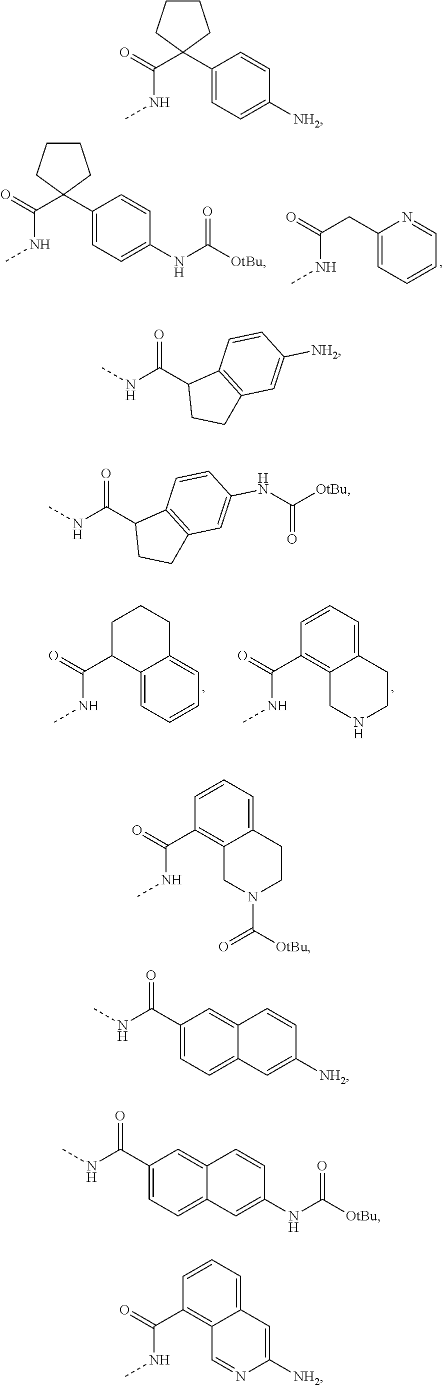

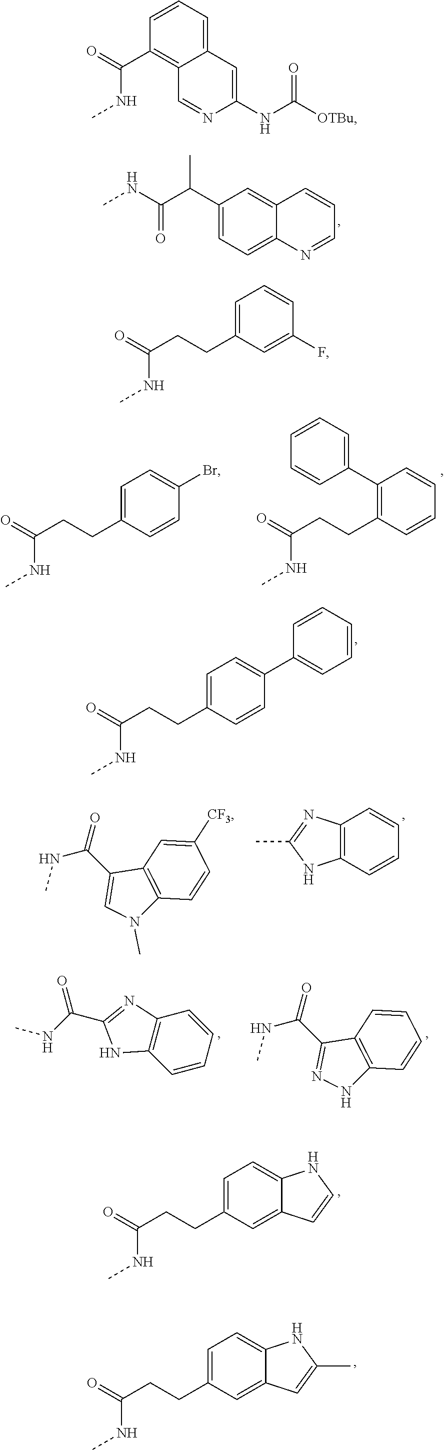

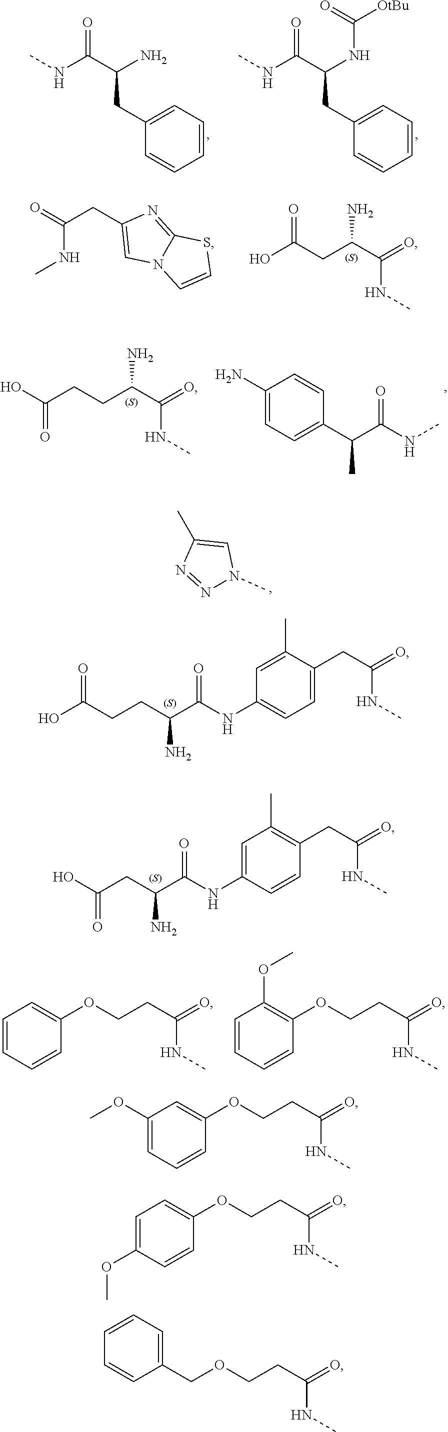

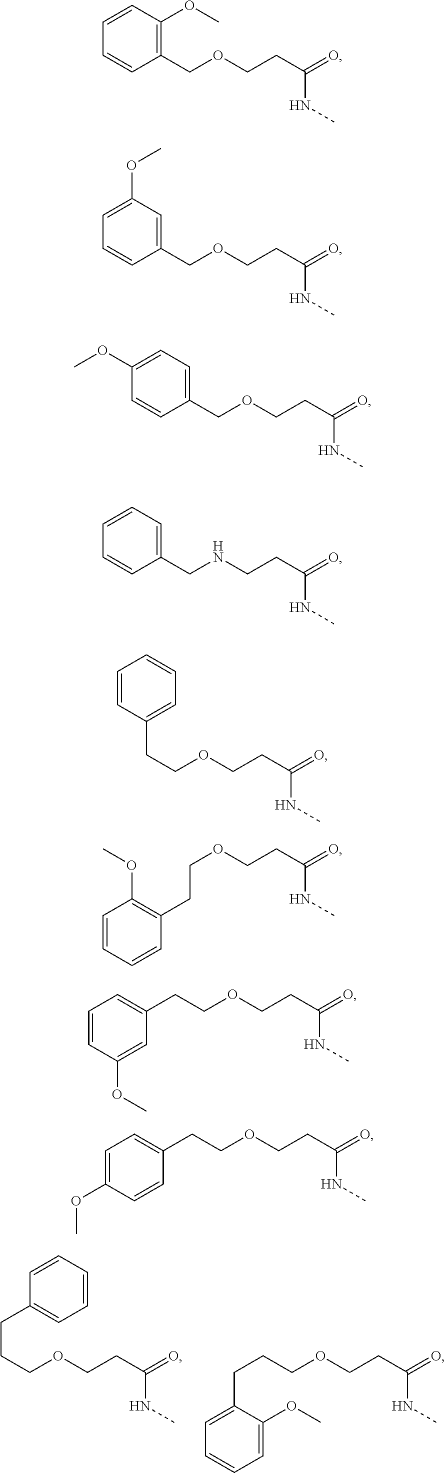

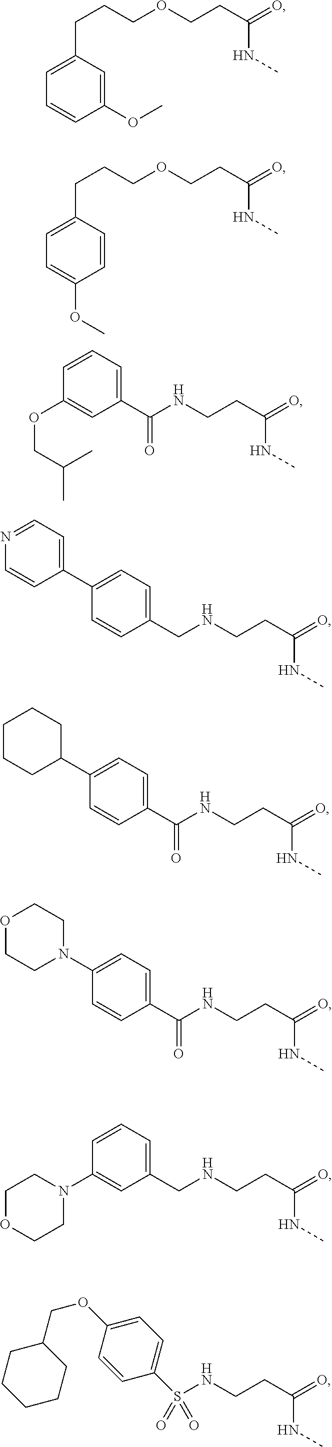

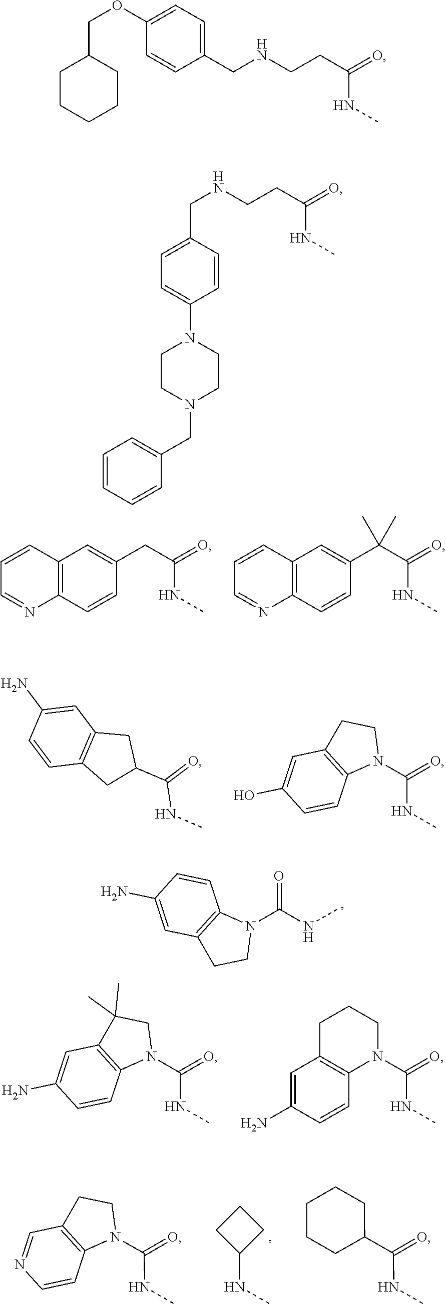

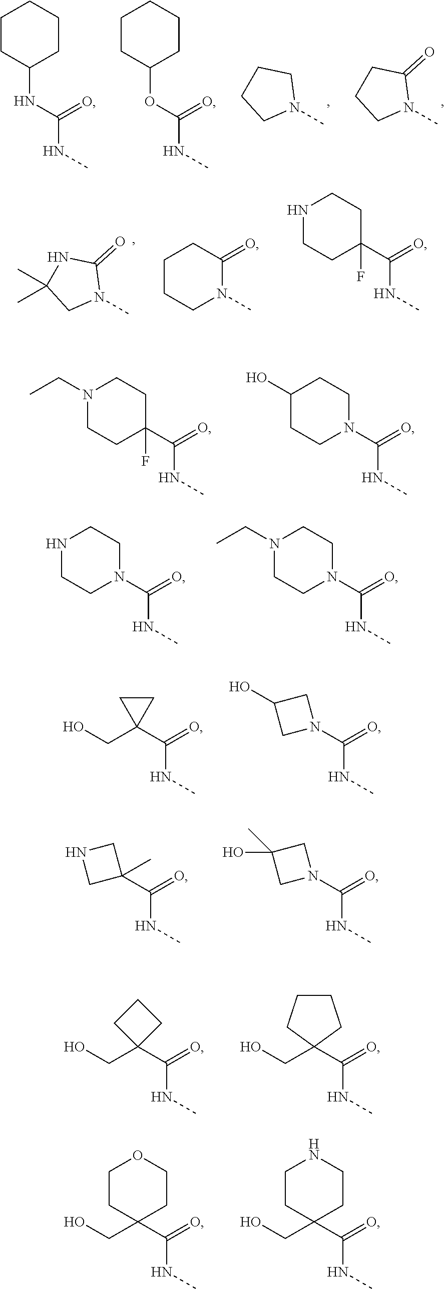

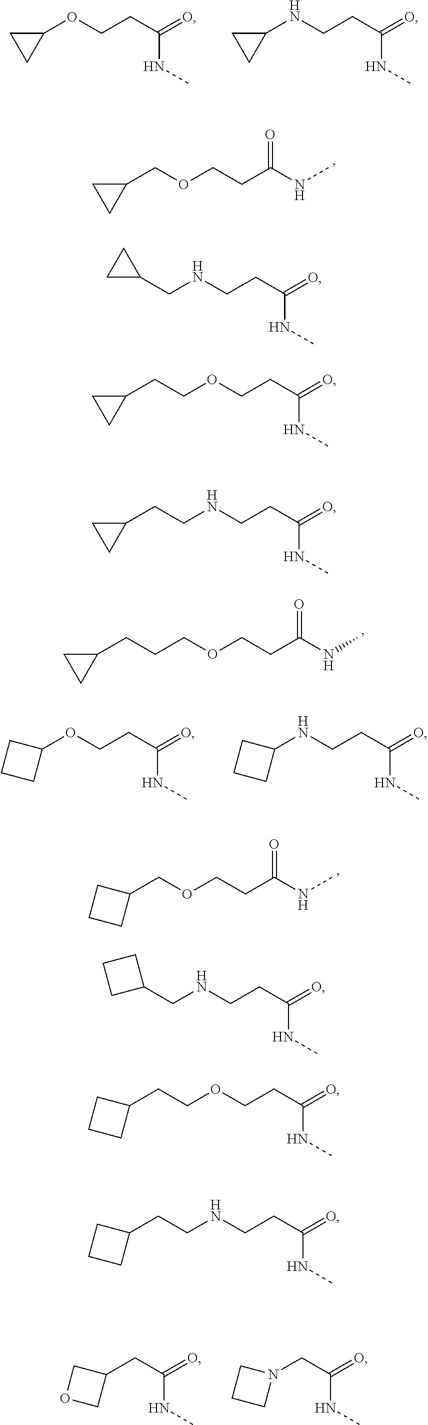

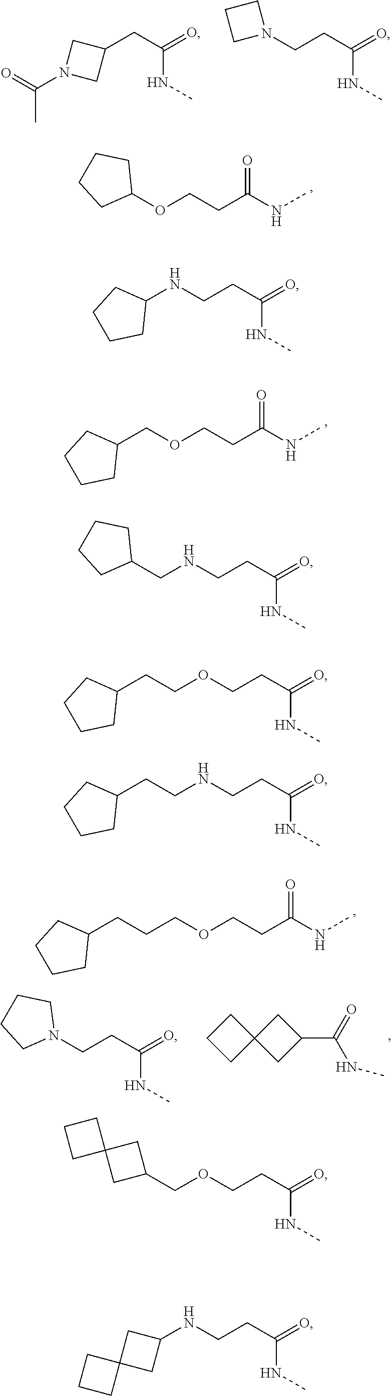

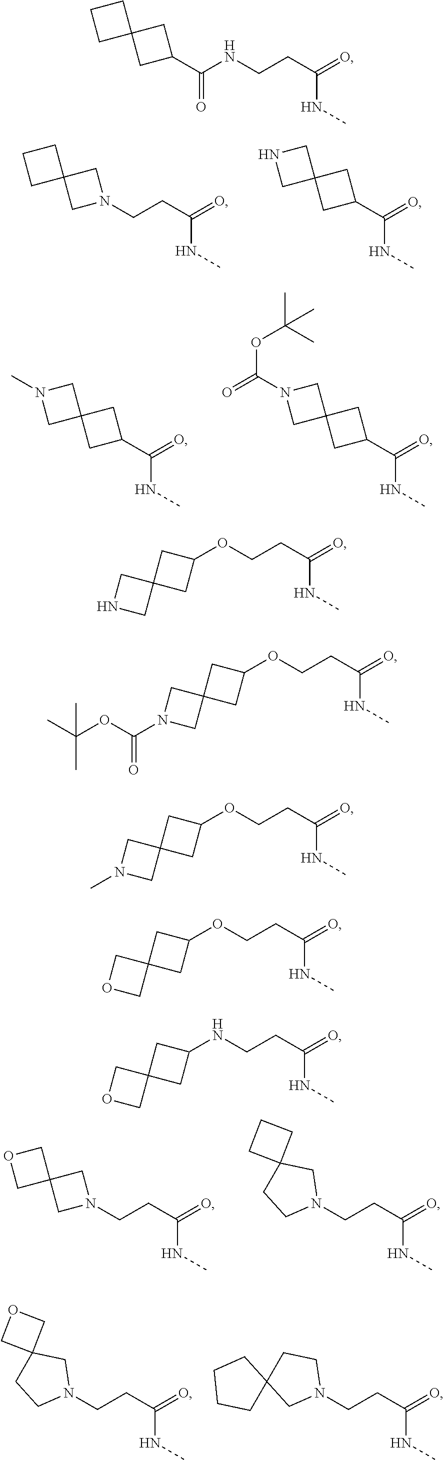

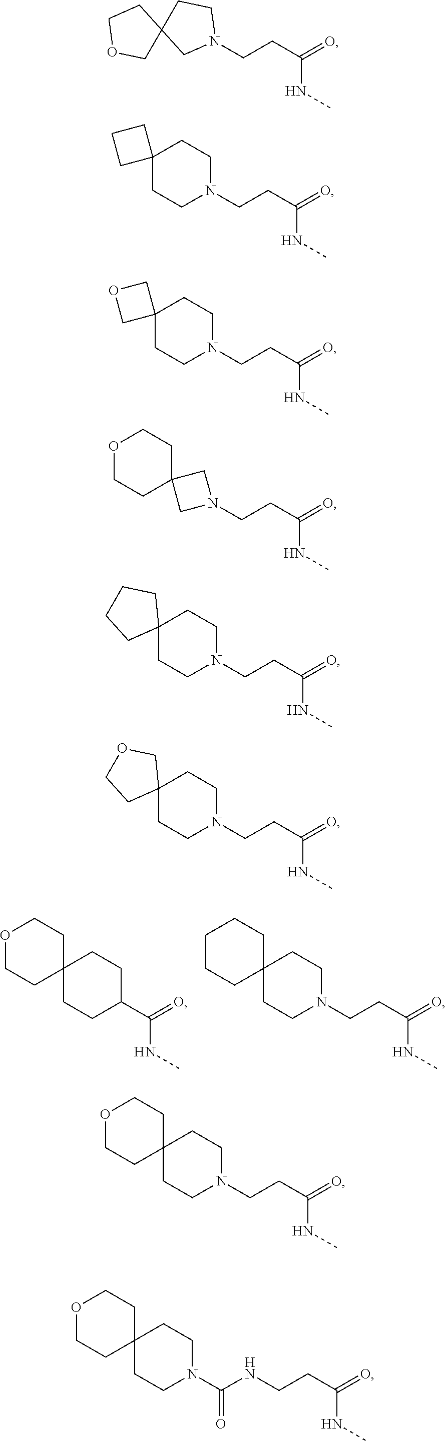

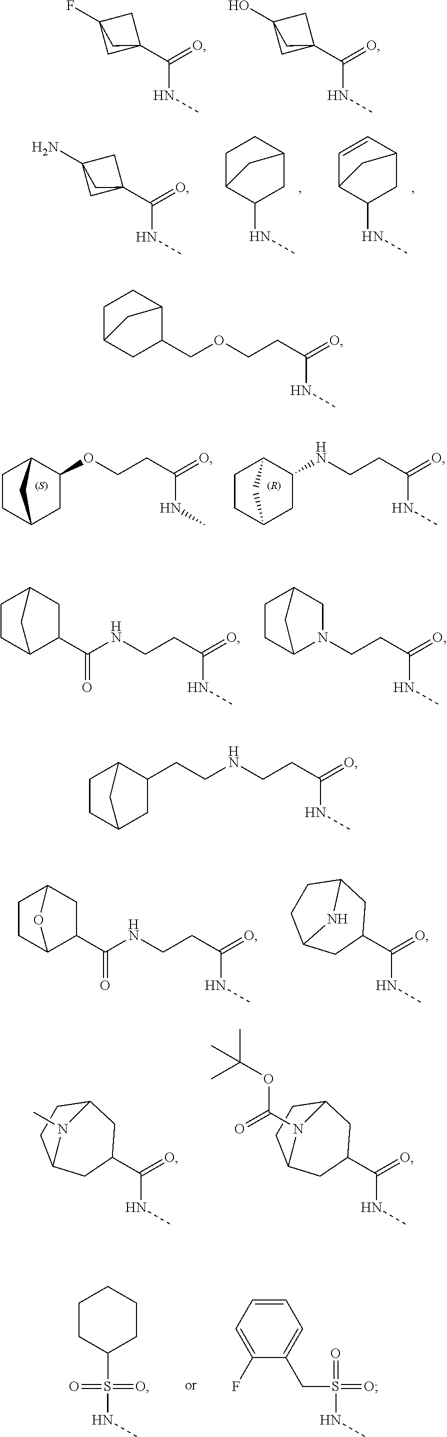

and/or
R¹ represents —CH₃, —CH₂CH₂—CH(CH₃)₂, —CH₂CH₂—C≡CH,

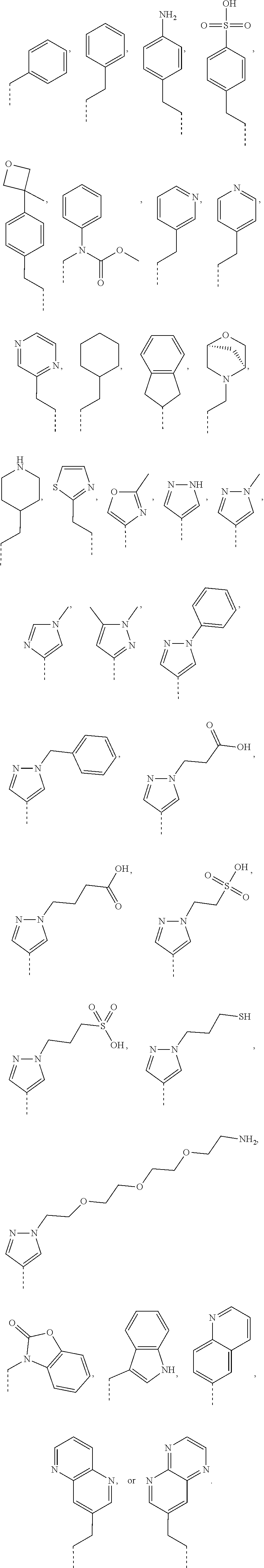

More preferred, the present invention relates to the compound of the formula (I),

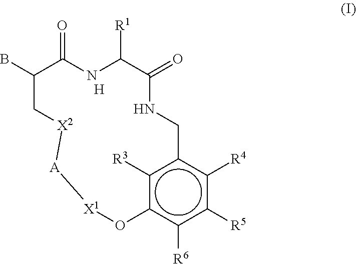

wherein
A represents —CO—NH—, —CO—NCH₃—,

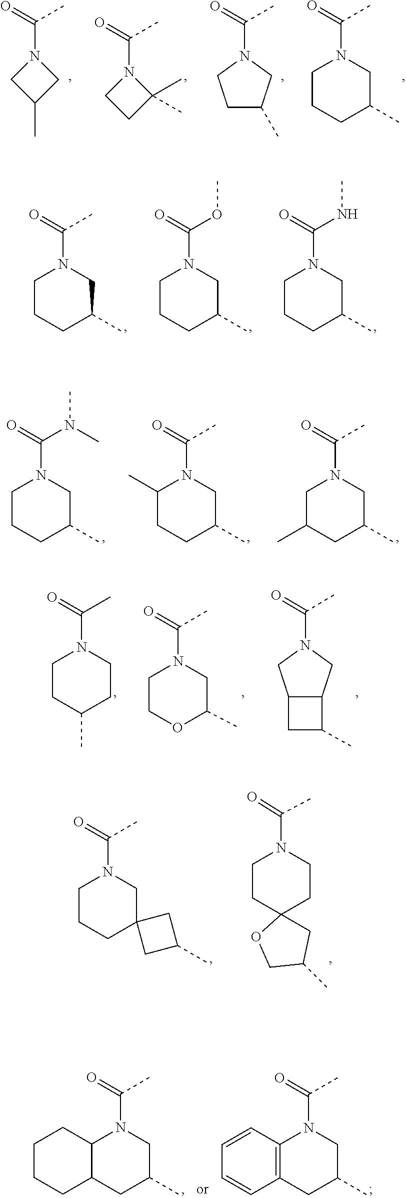

X₂ represents a bond, —CH₂—, —CH₂CH₂—, or —CH₂CH₂CH₂—;
X₁ represents a bond, —CH₂—, —CH₂CH₂—, —CH₂CH₂CH₂—, or —CH₂CH₂CH₂CH₂—;
B represents —H, —NH₂, —NHCOCH₃, —NHCOC(CH₃)₃, —NHCOC(CN)(CH₃)₂, —NHCOCH═CH₂, —NHCOCH₂C(CH₃)₃, —NHCOPh, —NHCOCH₂Ph, —NHCOCH₂—NH(CH₃), —NHCOCH₂—N(CH₃)CO₂tBu, —NHCOC(CH₃)₂CH₂OH, —NHCOC(CH₃)₂CH₂SH, —NHCOC(CH₃)₂CH₂NH₂, —NHCOC(CH₃)₂CH₂F, —NHCOC(CH₃)₂CF₃, —NHCOCF₂CH₂OH, —NHCOCF₂CH₂NH₂, —NHCOC(CH₂CH₃)₂CH₂OH, —NHCO₂CH₂CH₃, —NHCOC(CH₃)(CH₂OH)₂, —NHCOCH₂OCH₂CH₂—NH(CH₃), —NHCOCH₂OCH₂CH₂—N(CH₃)CO₂tBu, —NHCO(CH₂CH₂O)₂CH₃, —NHCO₂(CH₂CH₂O)₃CH₃, —NHCO₂(CH₂CH₂O)₅CH₃, —NHCO₂(CH₂CH₂O)₇CH₃, —NHCO₂CH₂Ph —NHCONHCH₂CH₃, —NHSO₂CH₃, —NHSO₂CH═CH₂, —NHSO₂CH₂Ph, —NHSO₂CH₂CH₂Ph, —NHSO₂CH₂CH₂CH₂Ph, —NHSO₂CH₂CF₃, —NHCH₂CF₃, —NHCH₂(CH₃)₂NH₂, —NHCH₂(CH₃)₂CH₂OH,

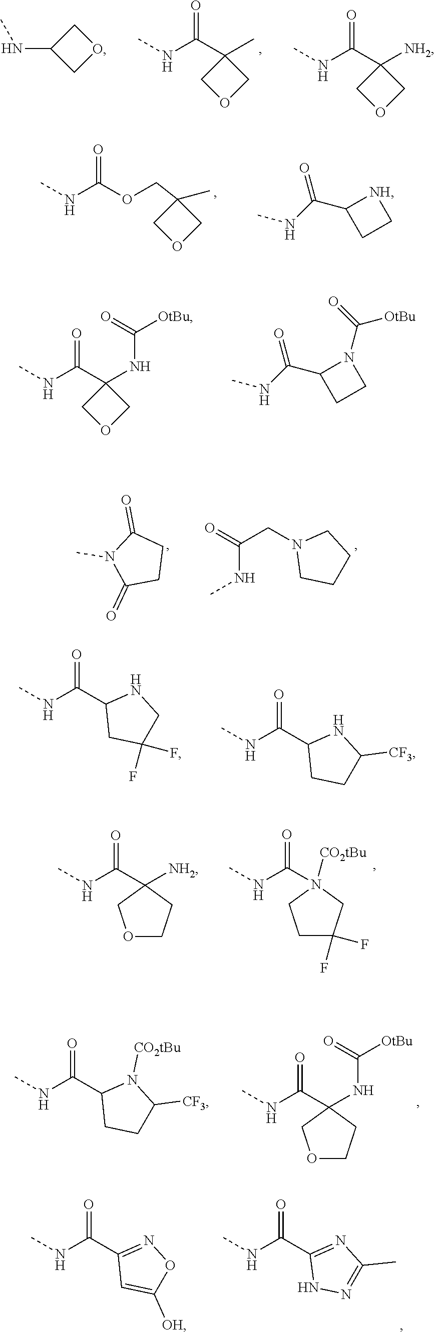

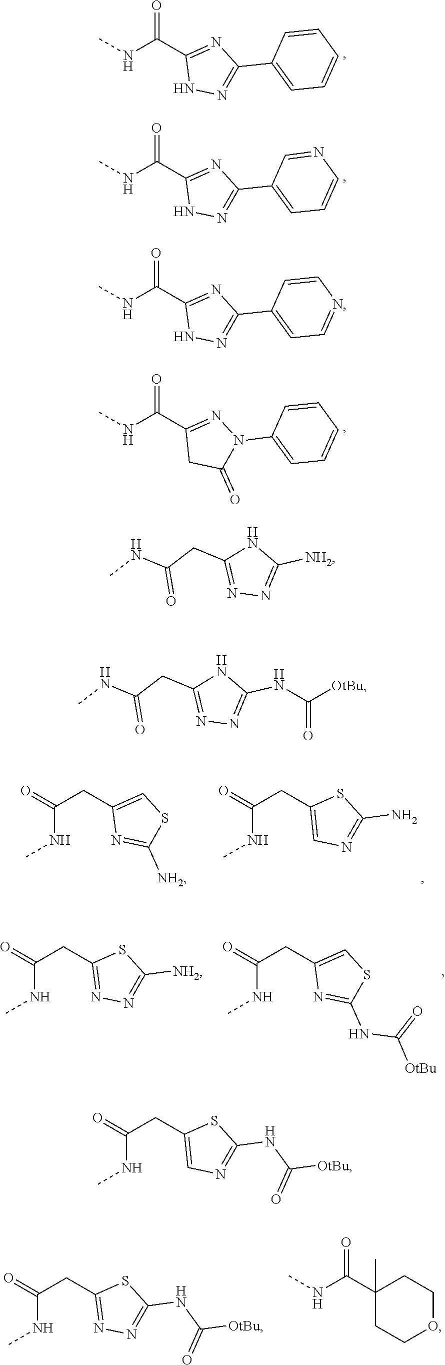

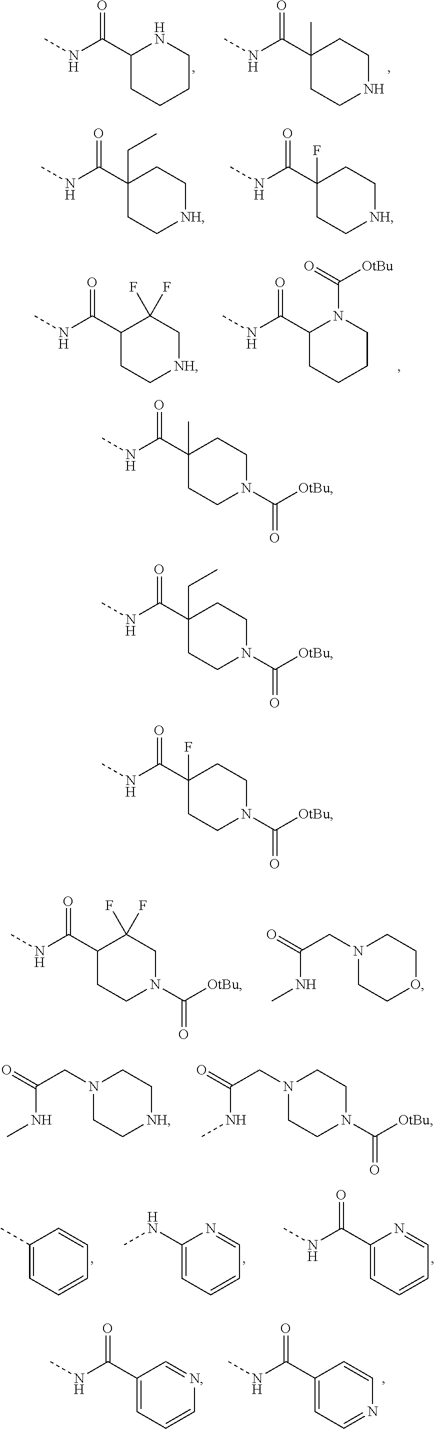

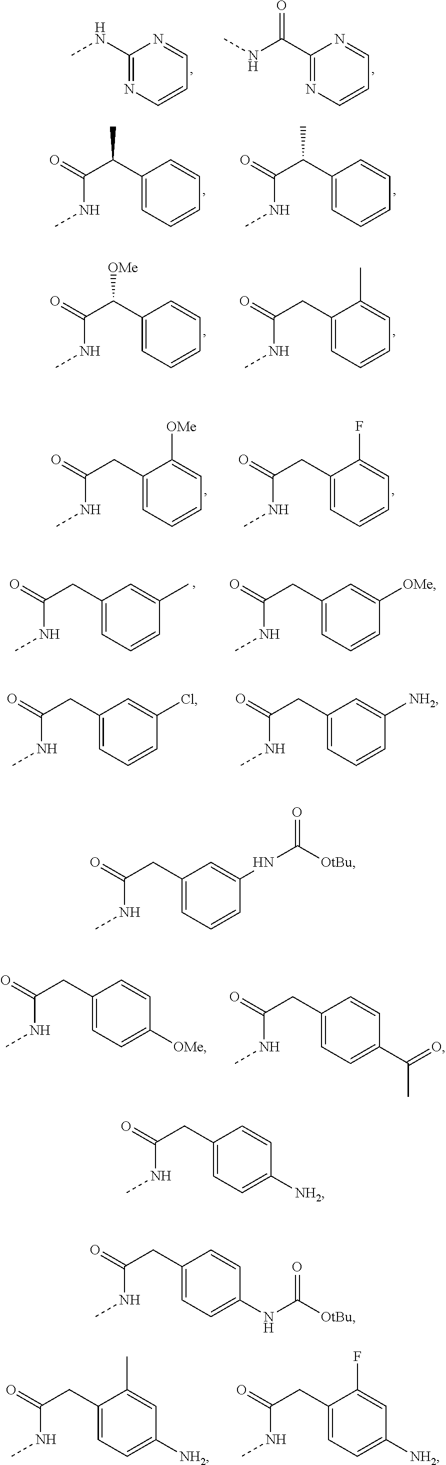

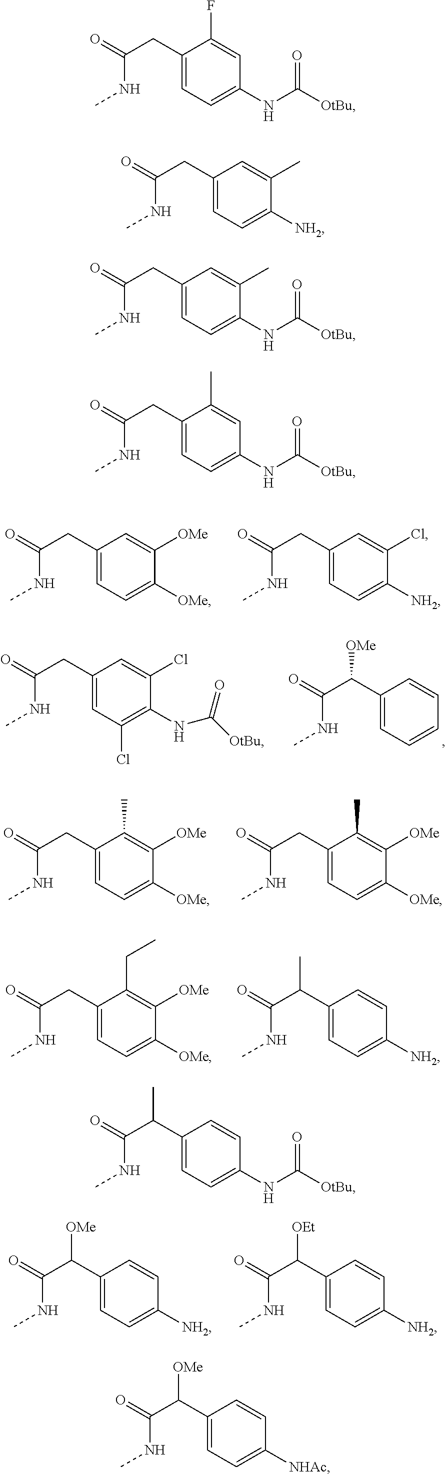

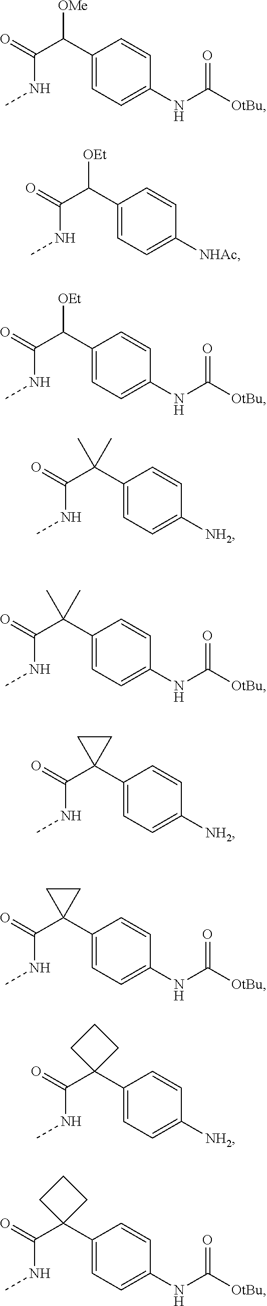

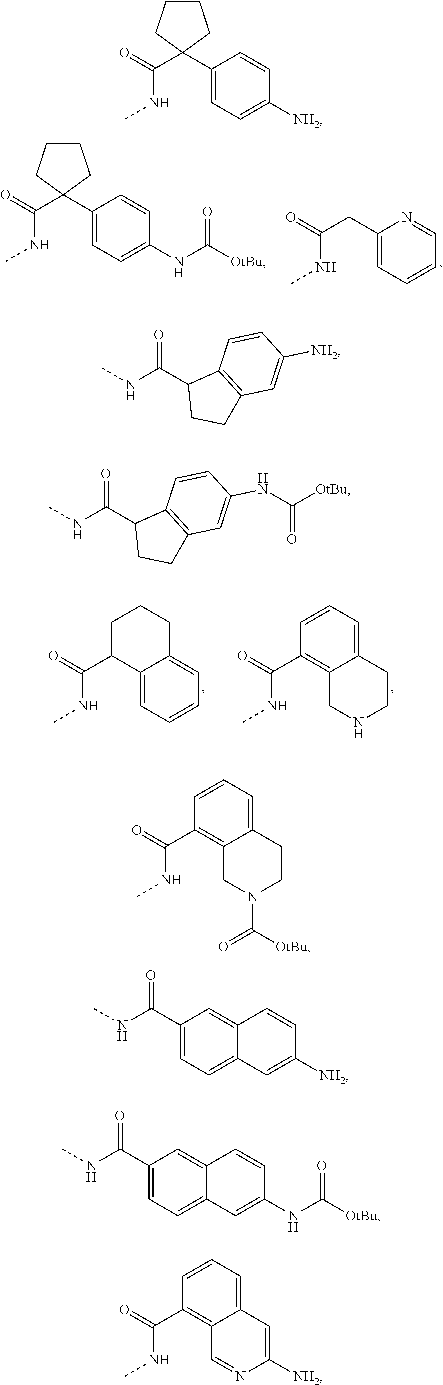

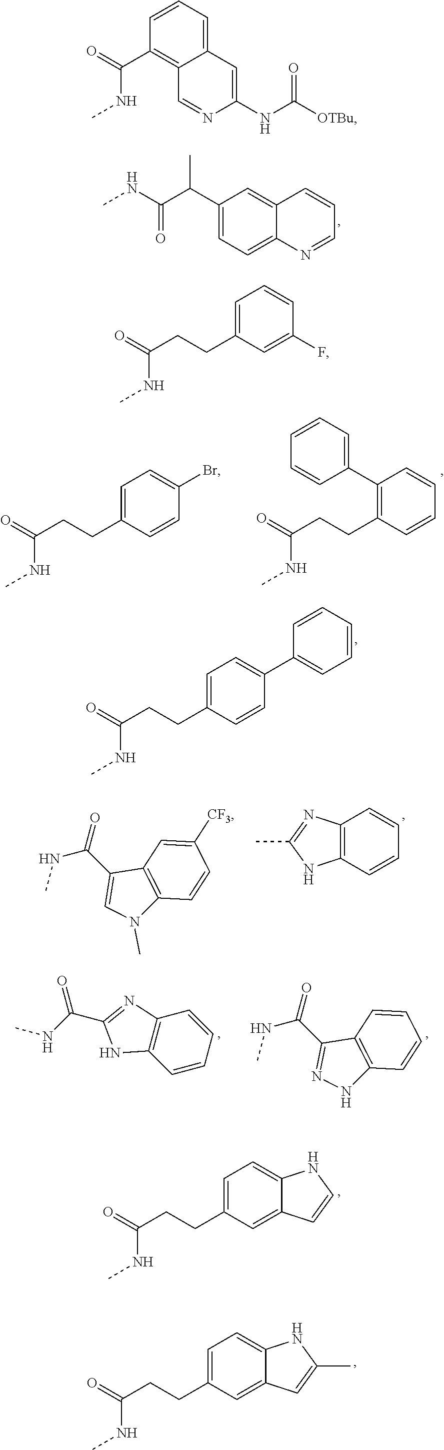

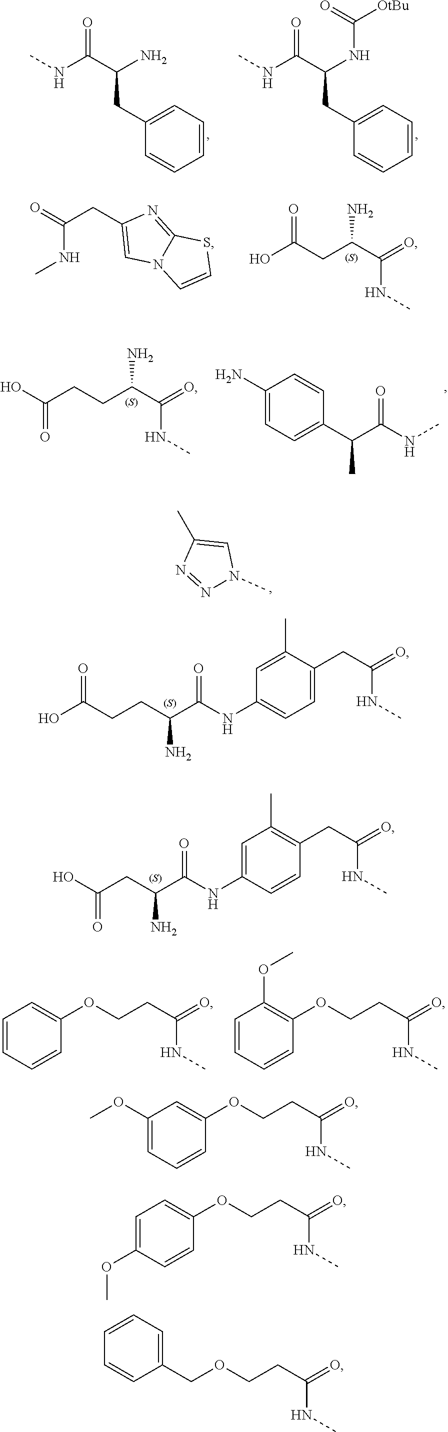

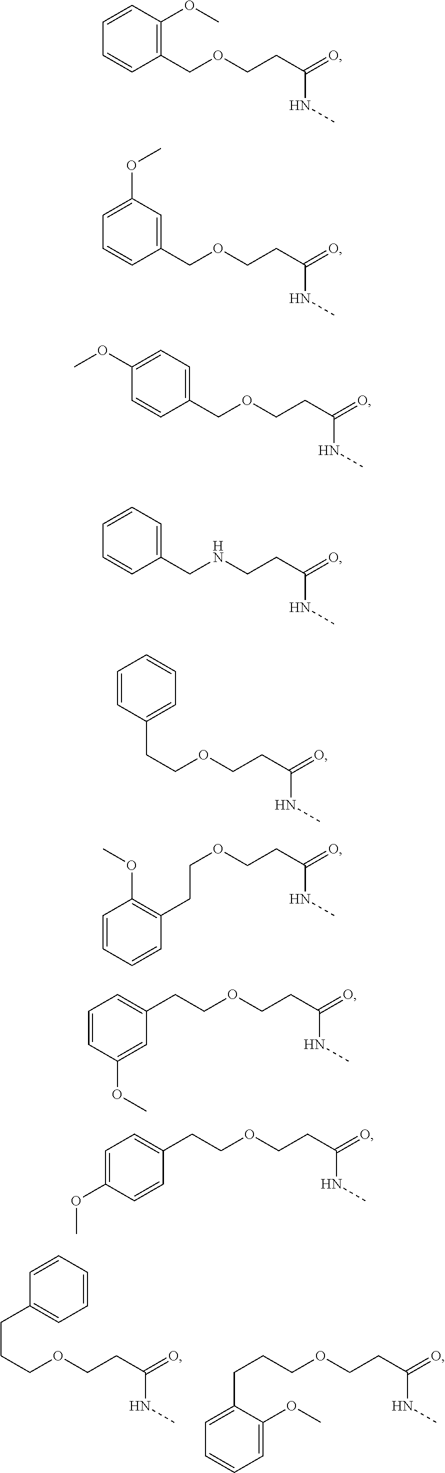

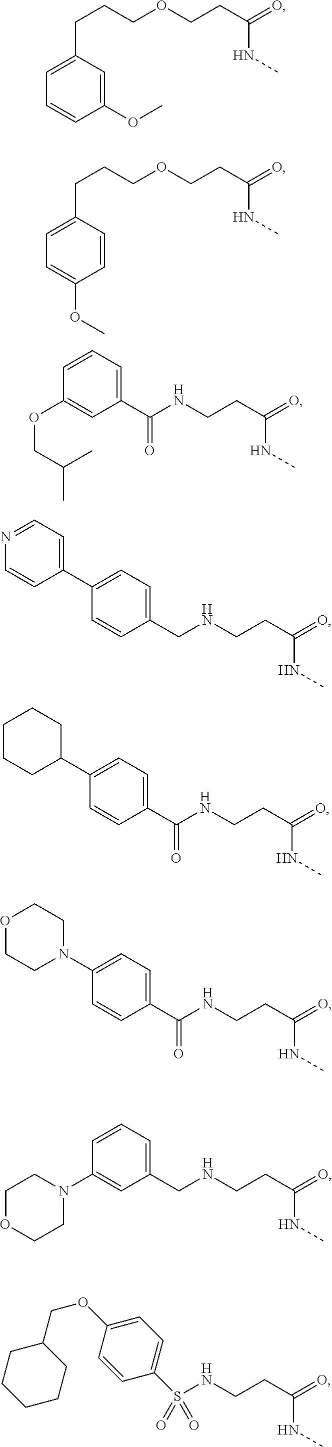

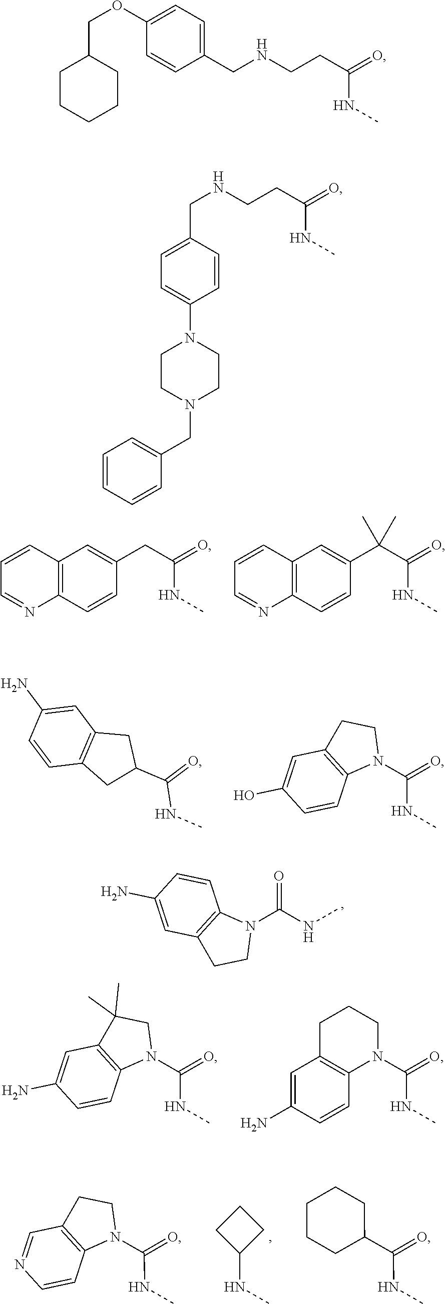

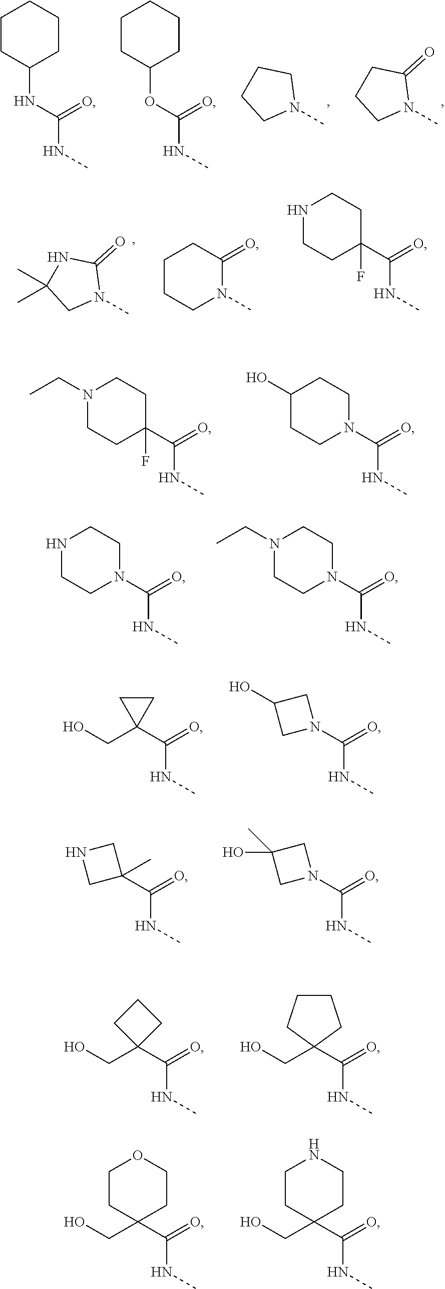

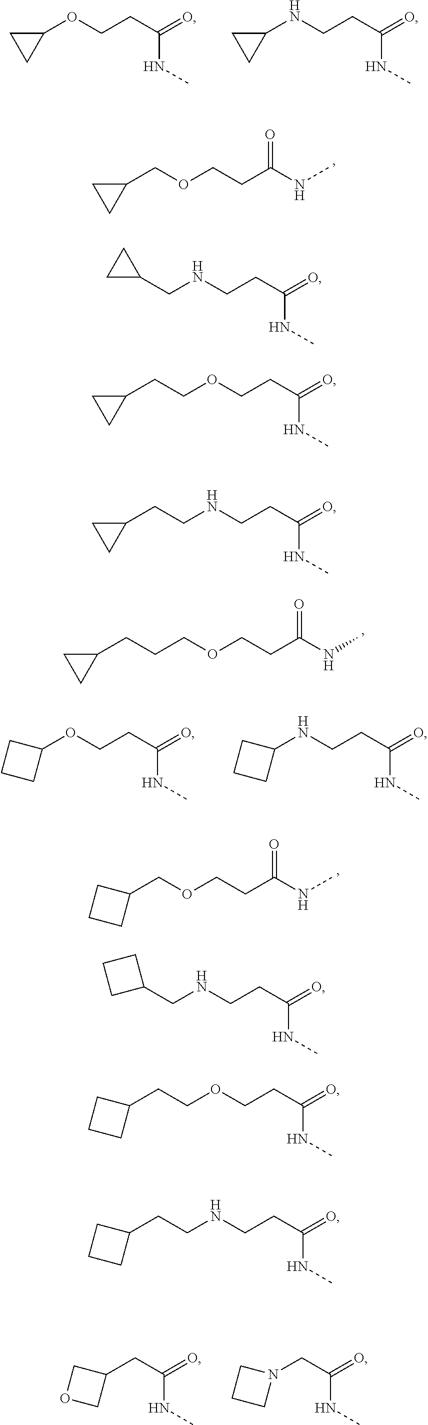

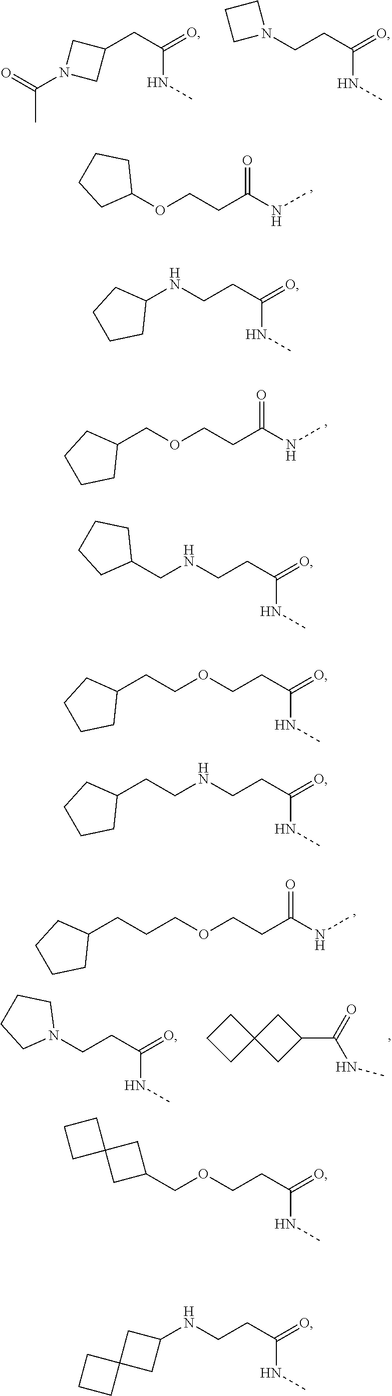

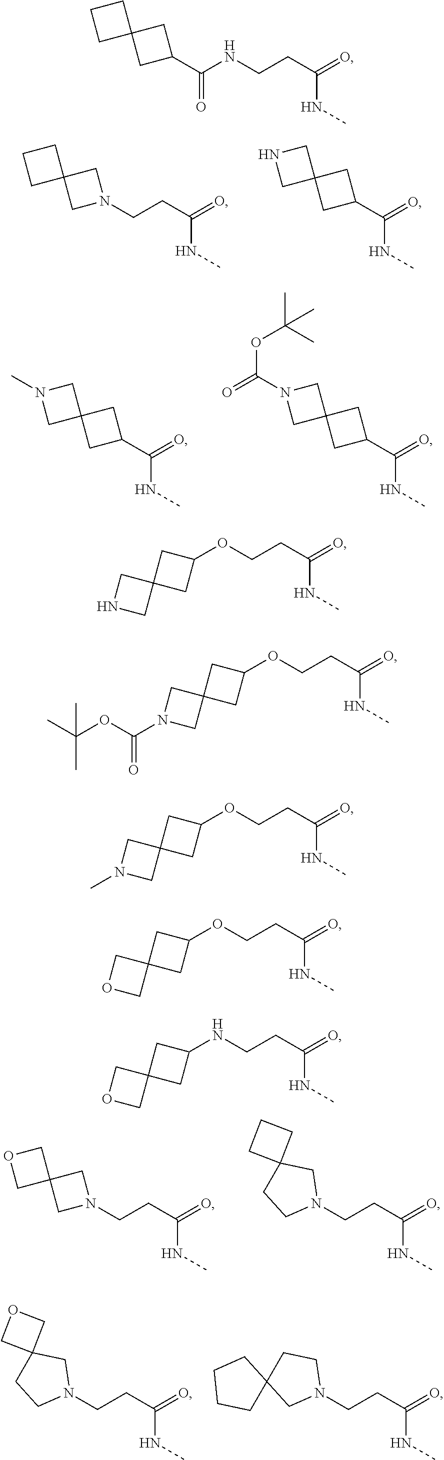

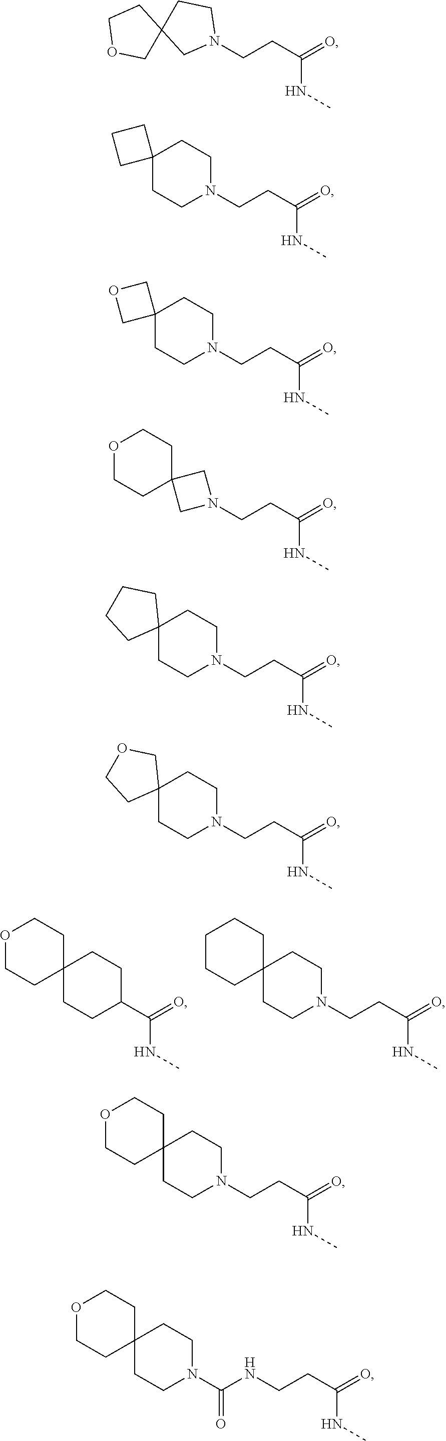

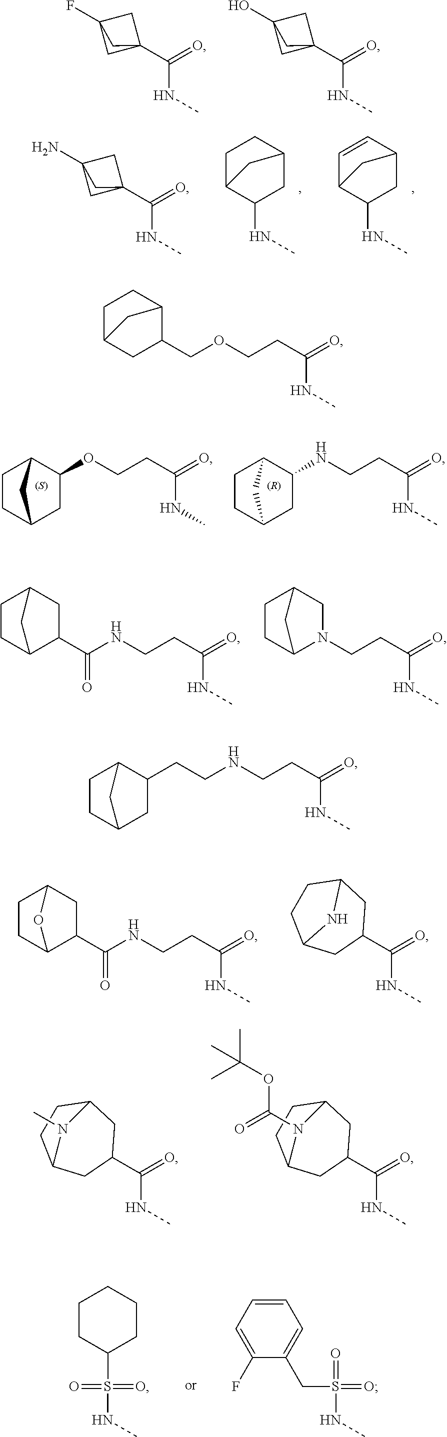

and
R¹ represents —CH₃, —CH₂CH₂—CH(CH₃)₂, —CH₂CH₂—C≡CH,

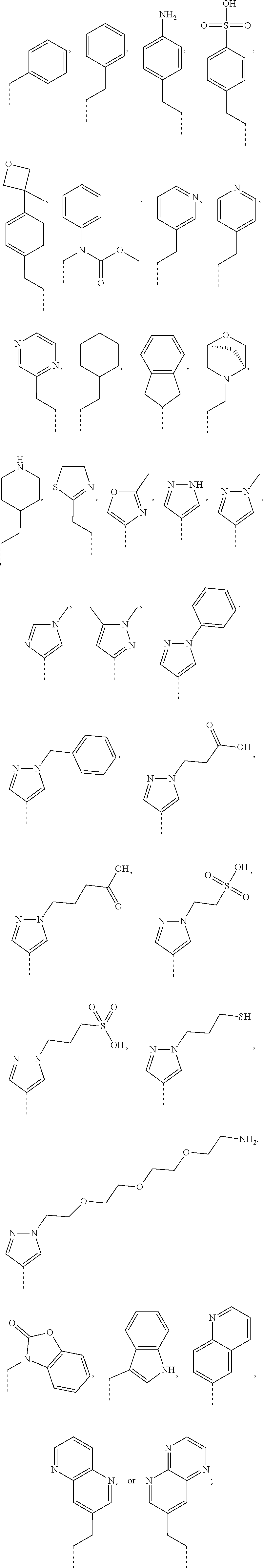

R³-R⁶ represent independently of each other —H, —CH₃, —OCH₃, —F, or —Cl;
or R⁵ and R⁶ may form together

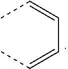

More preferred is the compound of the formula (I)

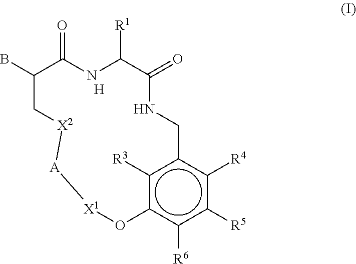

wherein
—X₂-A-X₁— represents

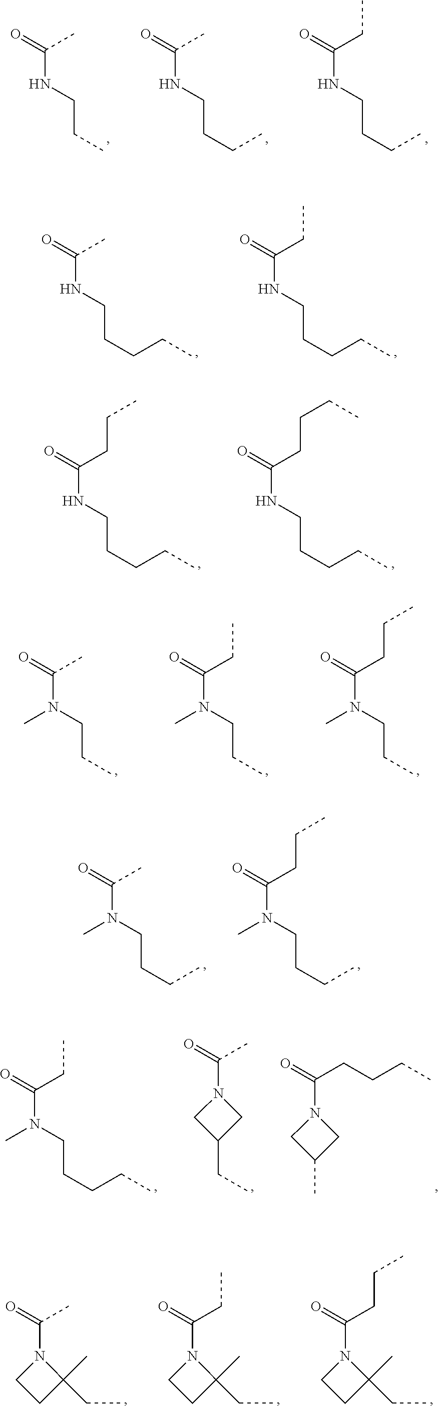

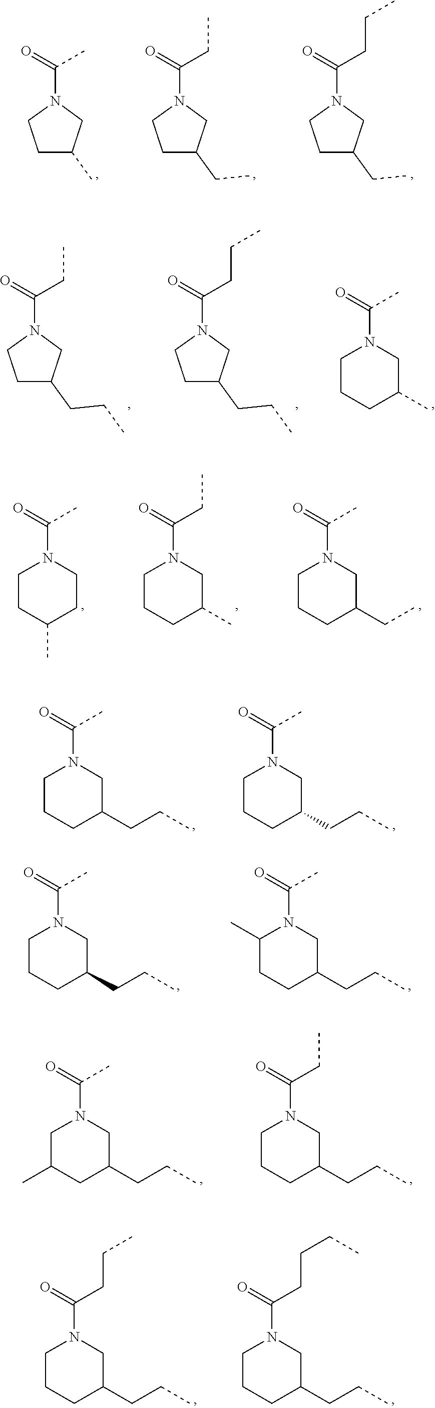

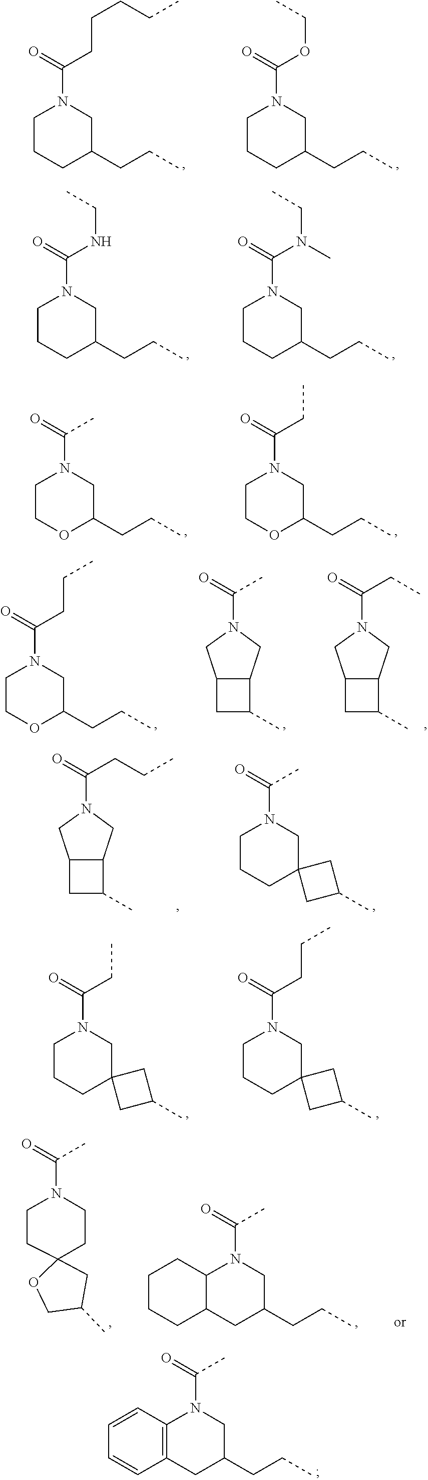

B represents —H, —NH₂, —NHCOCH₃, —NHCOC(CH₃)₃, —NHCOC(CN)(CH₃)₂, —NHCOCH═CH₂, —NHCOCH₂C(CH₃)₃, —NHCOPh, —NHCOCH₂Ph, —NHCOCH₂—NH(CH₃), —NHCOCH₂—N(CH₃)CO₂tBu, —NHCOC(CH₃)₂CH₂OH, —NHCOC(CH₃)₂CH₂SH, —NHCOC(CH₃)₂CH₂NH₂, —NHCOC(CH₃)₂CH₂F, —NHCOC(CH₃)₂CF₃, —NHCOCF₂CH₂OH, —NHCOCF₂CH₂NH₂, —NHCOC(CH₂CH₃)₂CH₂OH, —NHCOC(CH₃)(CH₂OH)₂, —NHCOCH₂OCH₂CH₂—NH(CH₃), —NHCOCH₂OCH₂CH₂—N(CH₃)CO₂tBu, —NHCO(CH₂CH₂O)₂CH₃, —NHCO₂CH₂CH₃, —NHCO₂(CH₂CH₂O)₃CH₃, —NHCO₂(CH₂CH₂O)₅CH₃, —NHCO₂(CH₂CH₂O)₇CH₃, —NHCO₂CH₂Ph —NHCONHCH₂CH₃, —NHSO₂CH₃, —NHSO₂CH═CH₂, —NHSO₂CH₂Ph, —NHSO₂CH₂CH₂Ph, —NHSO₂CH₂CH₂CH₂Ph, —NHSO₂CH₂CF₃, —NHCH₂CF₃, —NHCH₂(CH₃)₂NH₂, —NHCH₂(CH₃)₂CH₂OH,

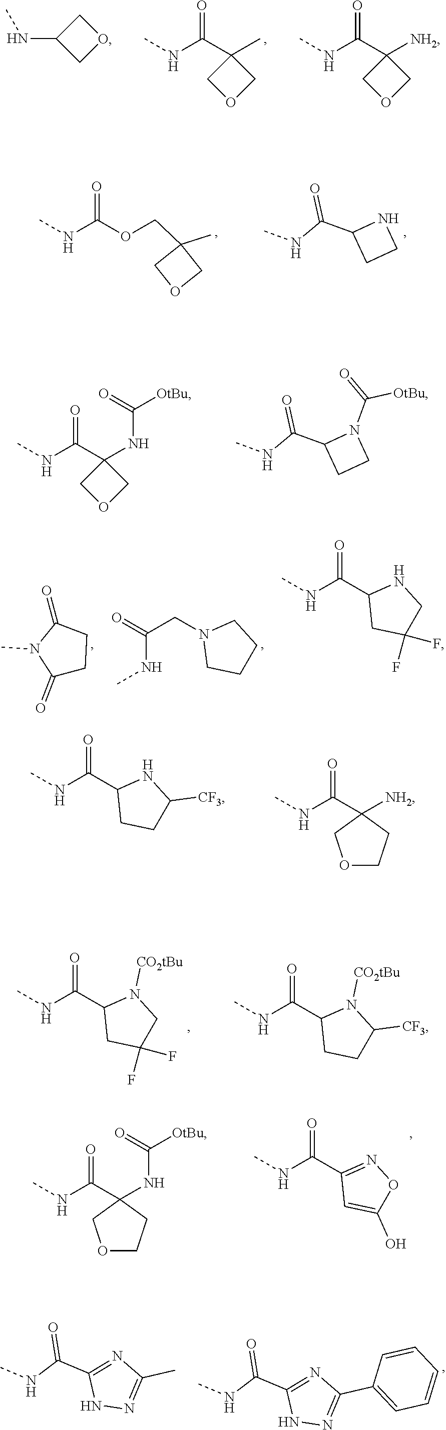

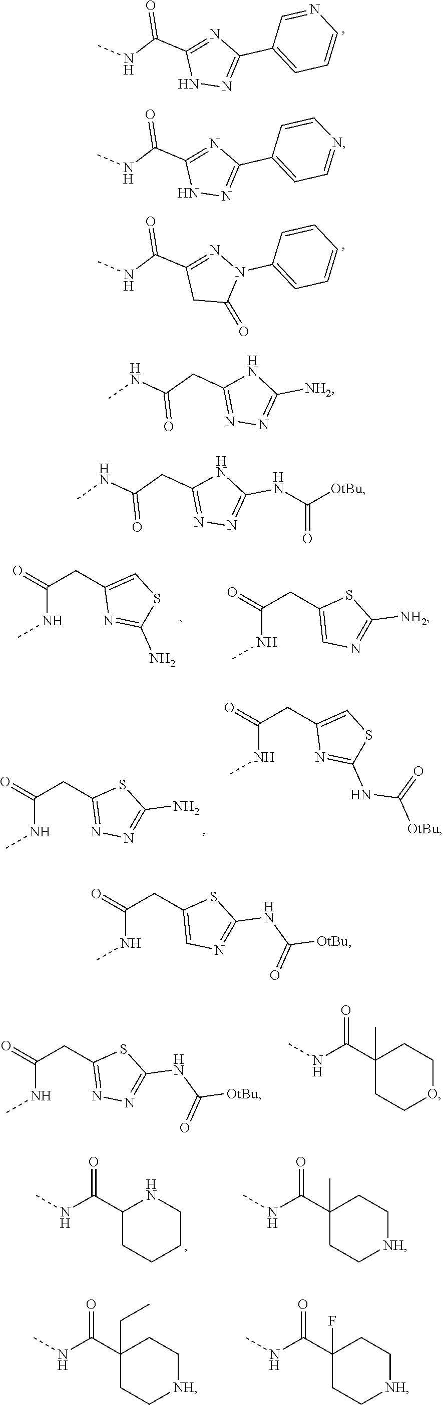

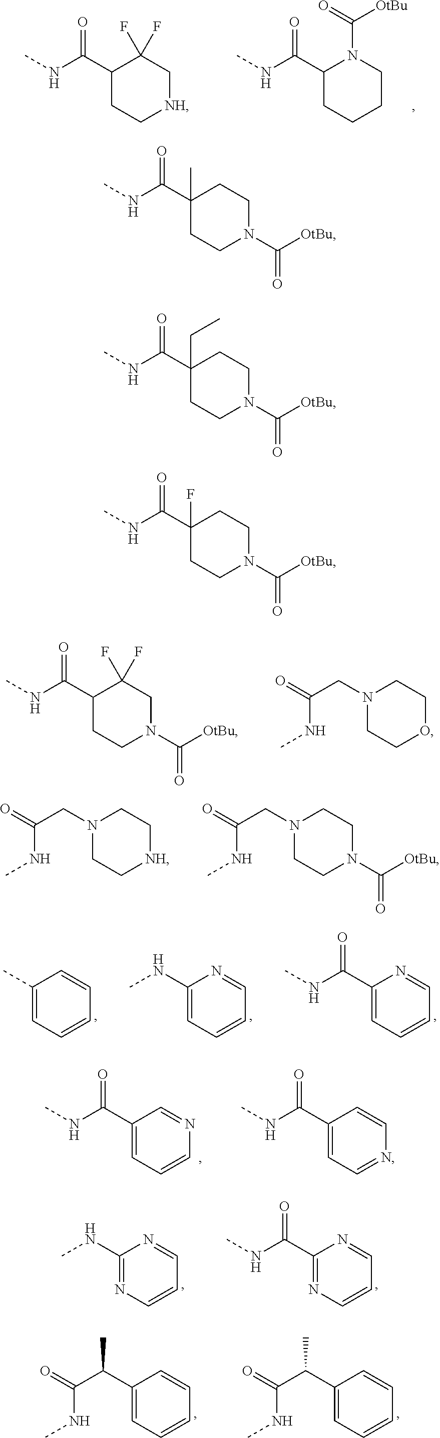

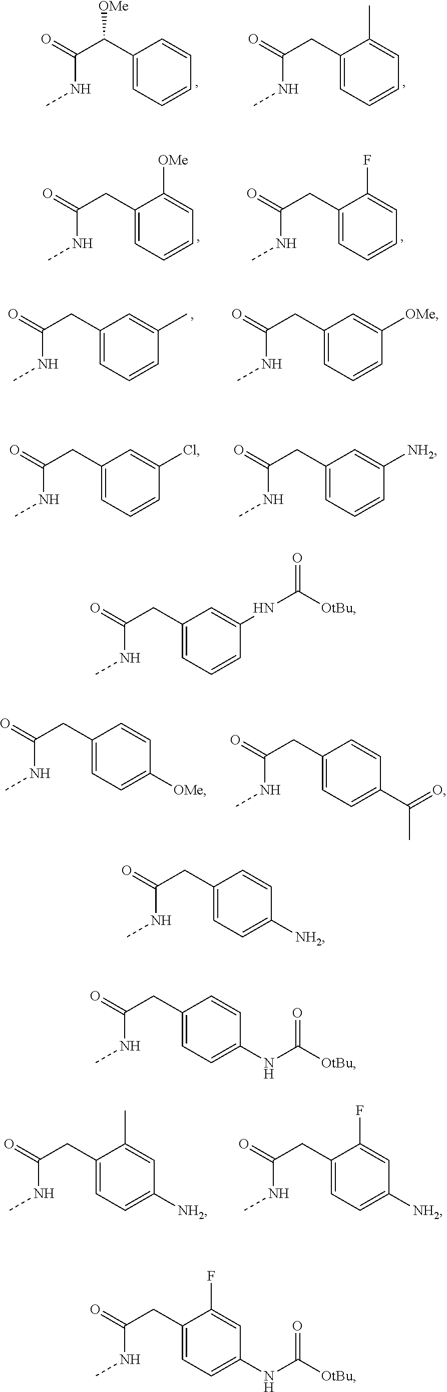

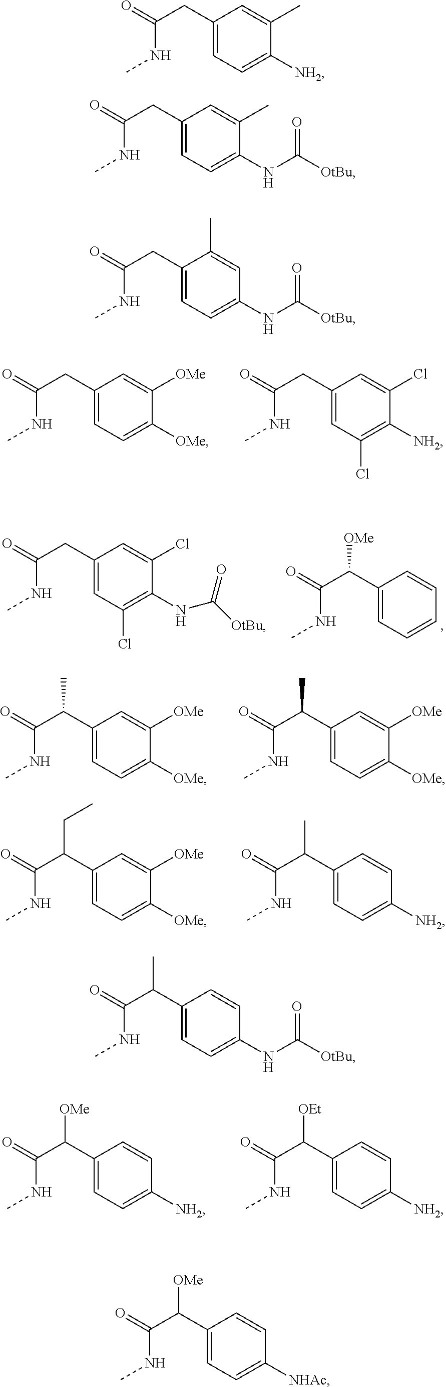

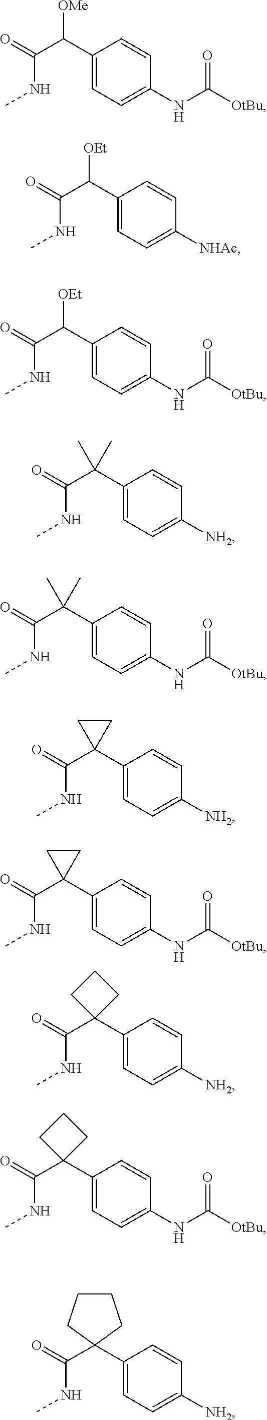

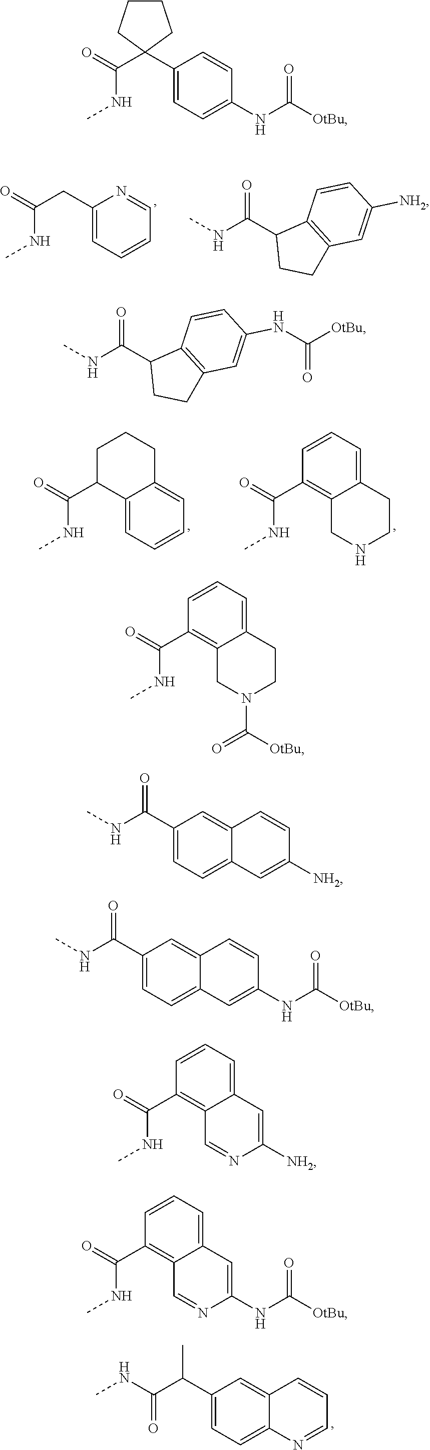

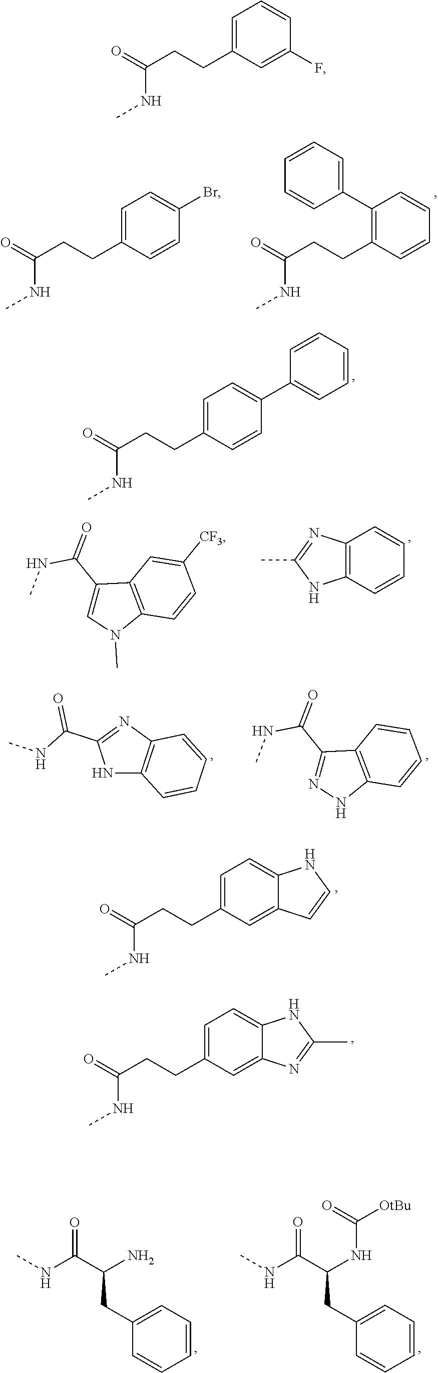

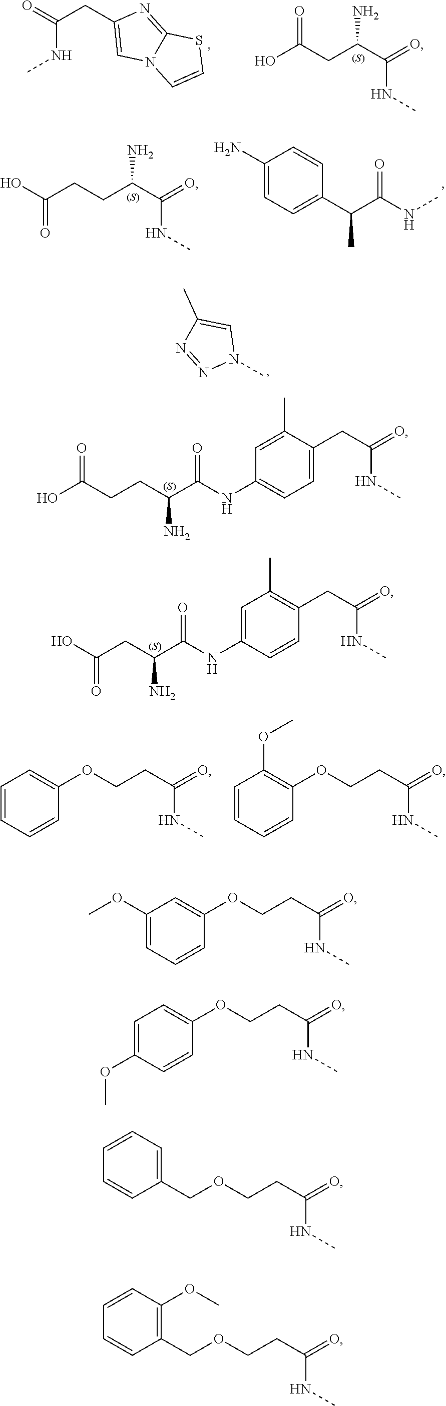

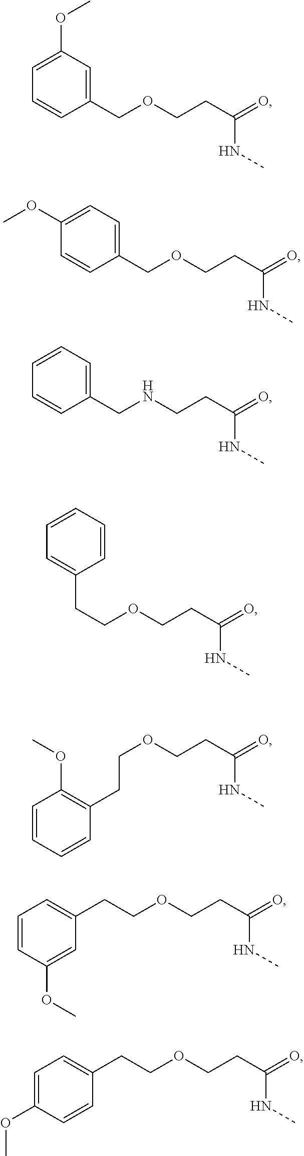

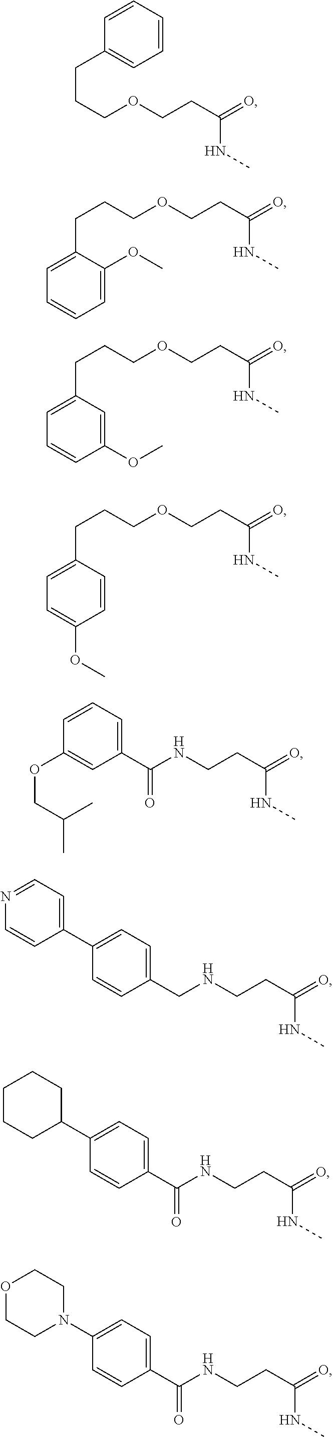

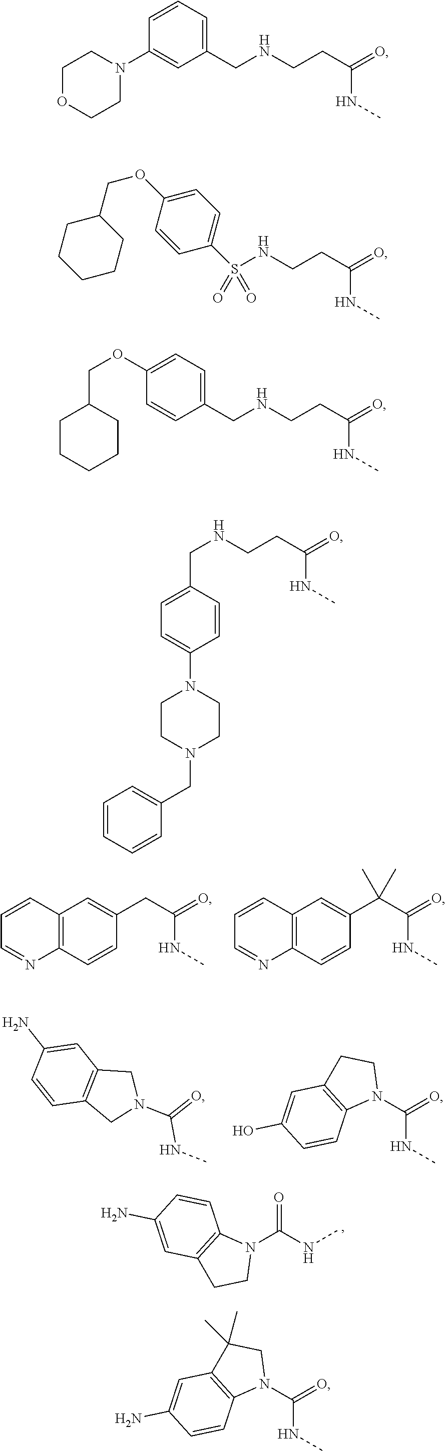

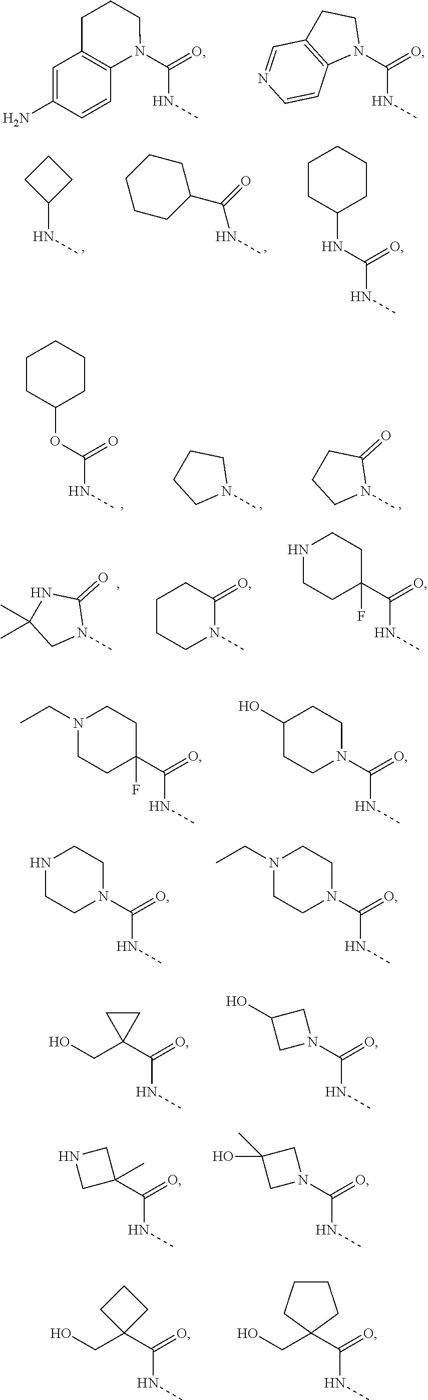

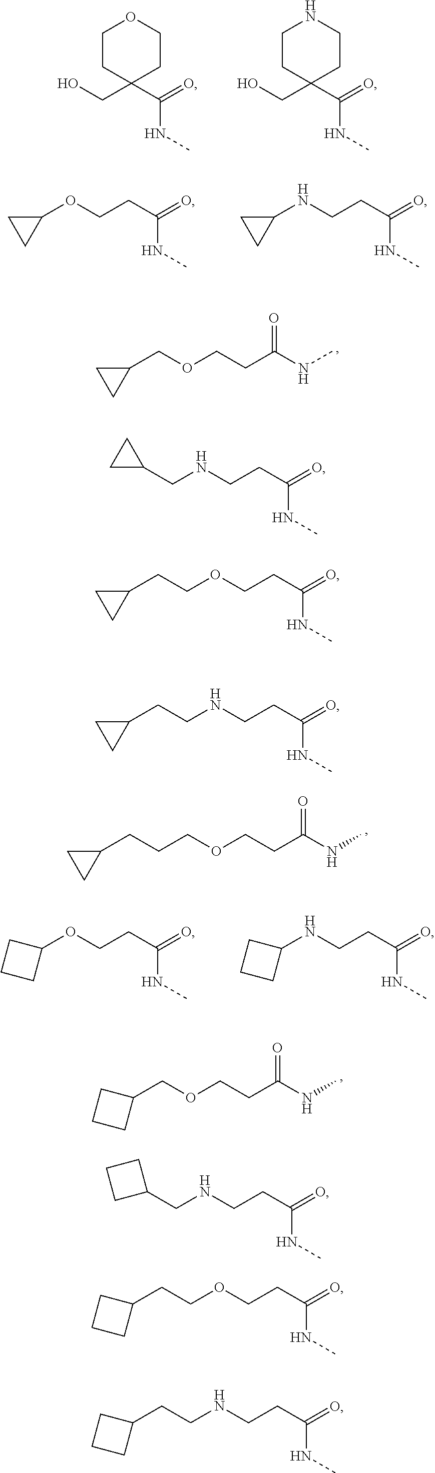

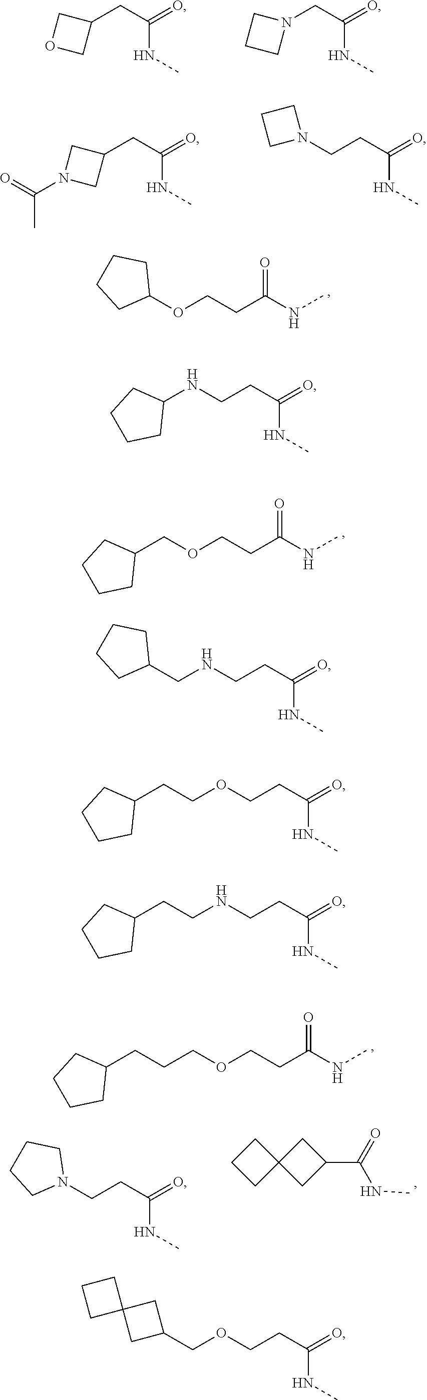

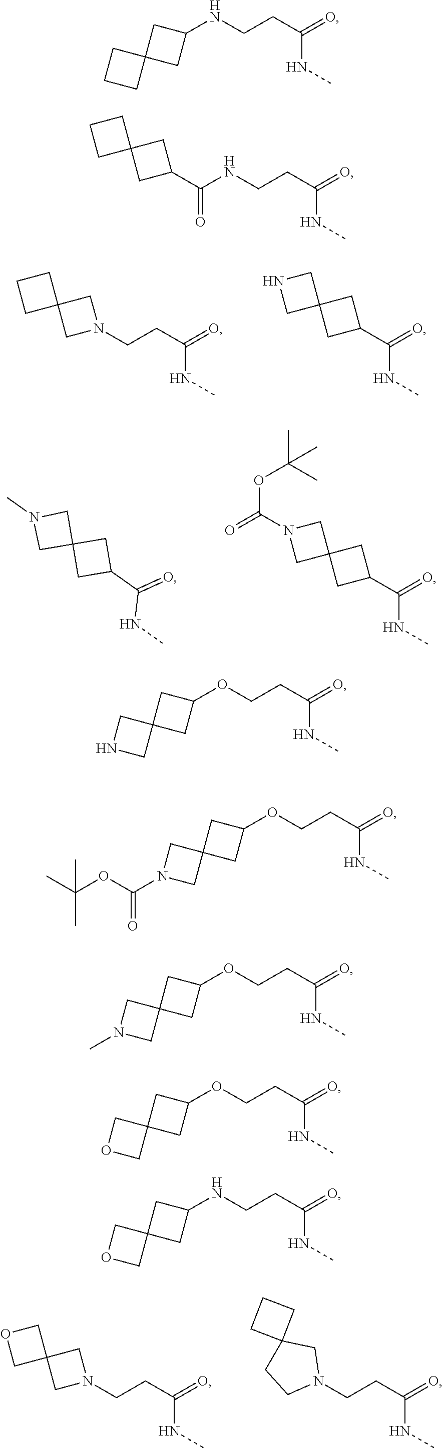

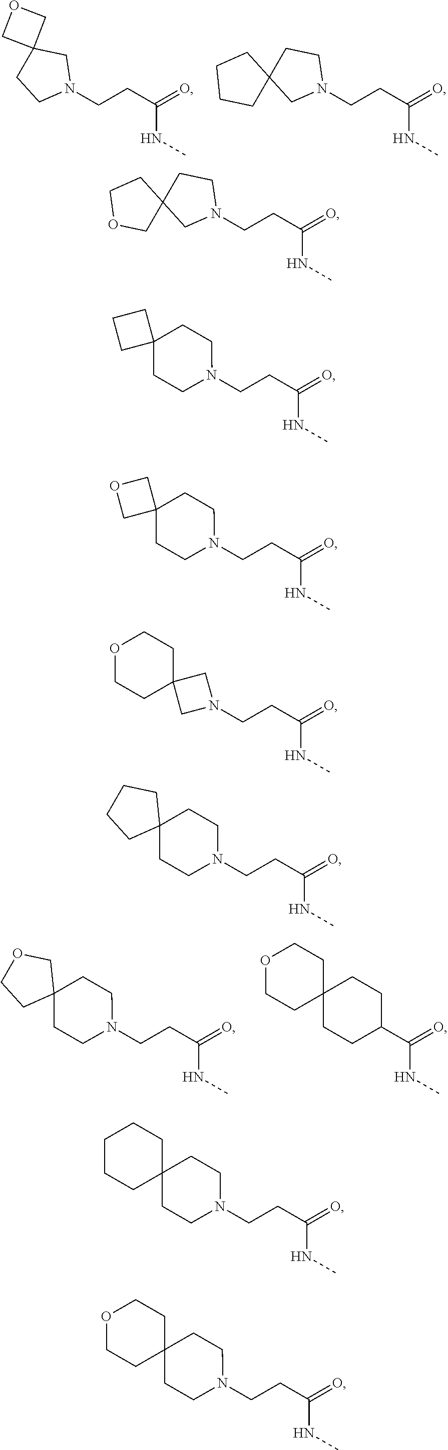

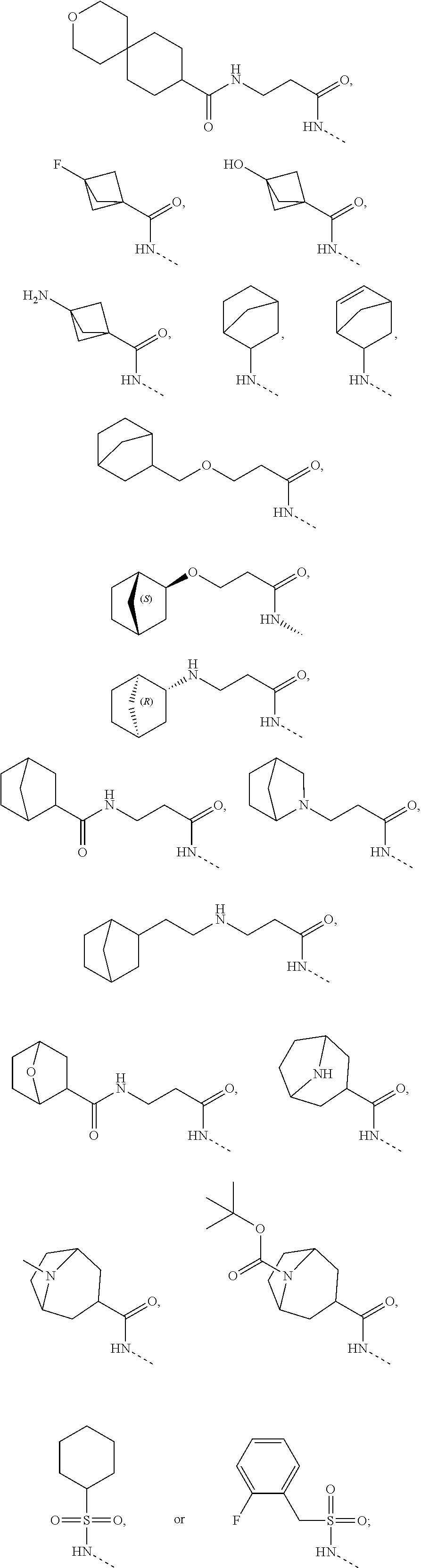

and
R¹ represents —CH₃, —CH₂CH₂—CH(CH₃)₂, —CH₂CH₂—C≡CH,

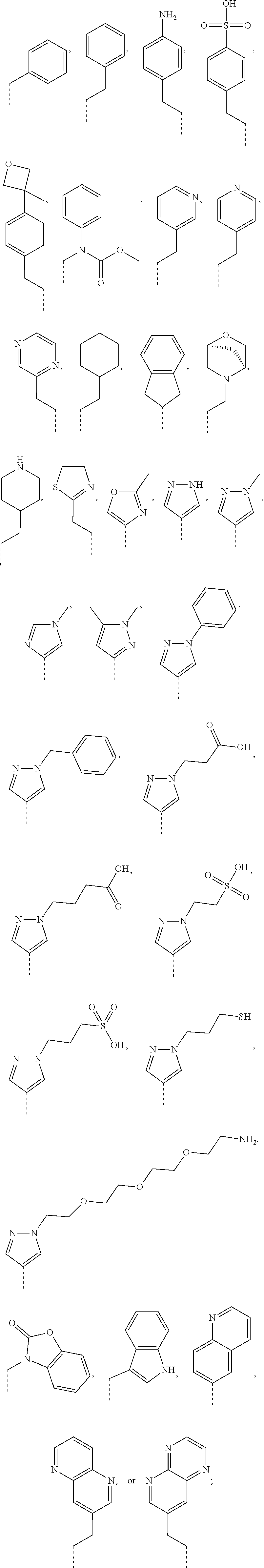

R³-R⁶ represent independently of each other —H, —CH₃, —OCH₃, —F, or —Cl;
or R⁵ and R⁶ may form together

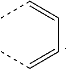

In some embodiments, the present invention relates to the compound having any one of the formulae (II-1)-(II-16):

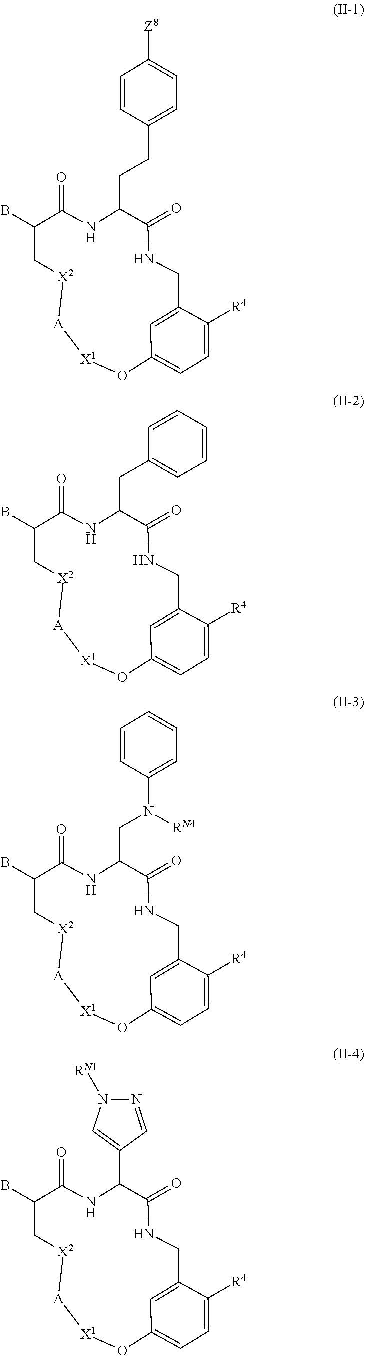

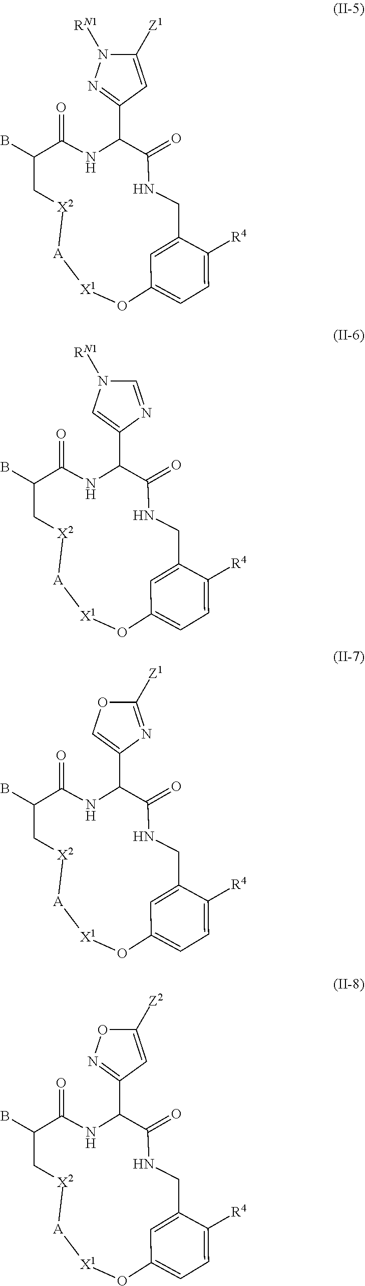

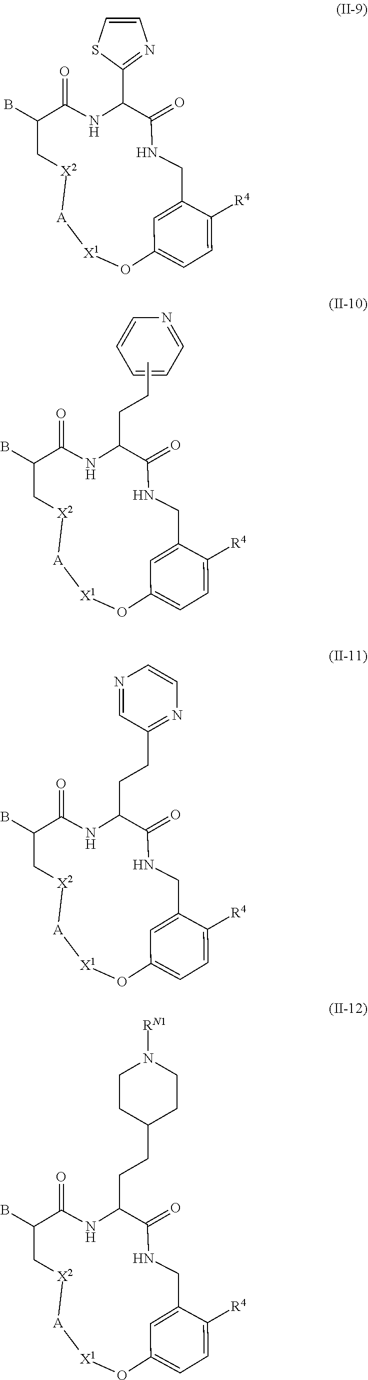

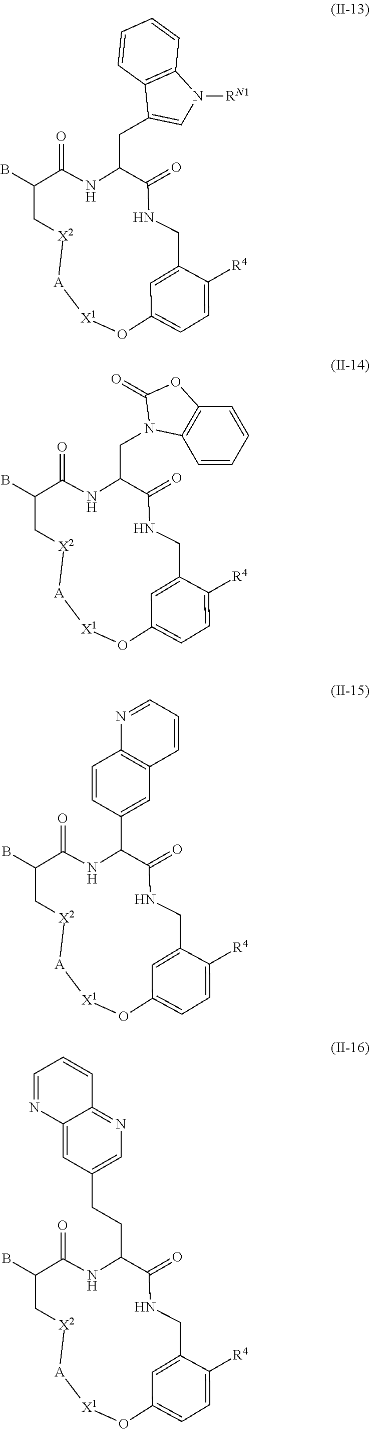

wherein A, B, R⁴, R^{N1}, R^{N4}, X¹, X², Z¹, Z², and Z^{a} have the same meanings as defined above.

In some embodiments, the present invention relates to the compound having any one of the formulae (III-1)-(III-10):

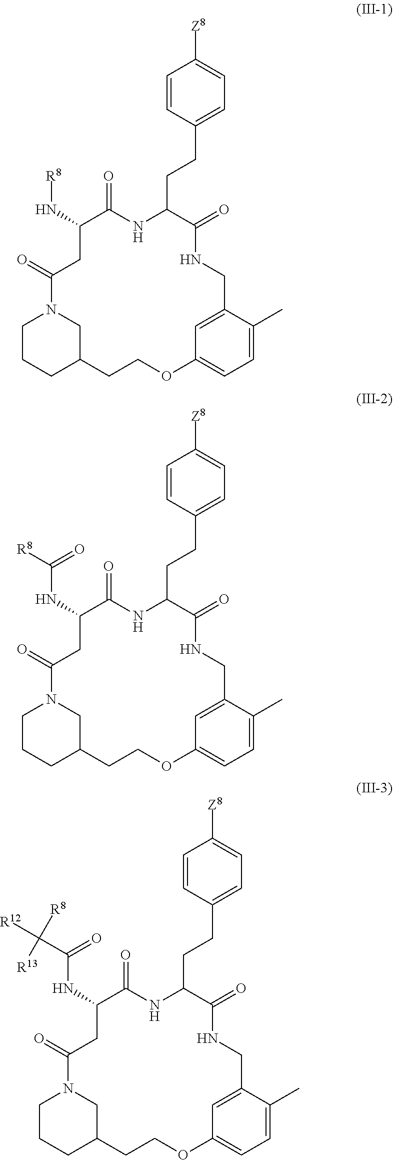

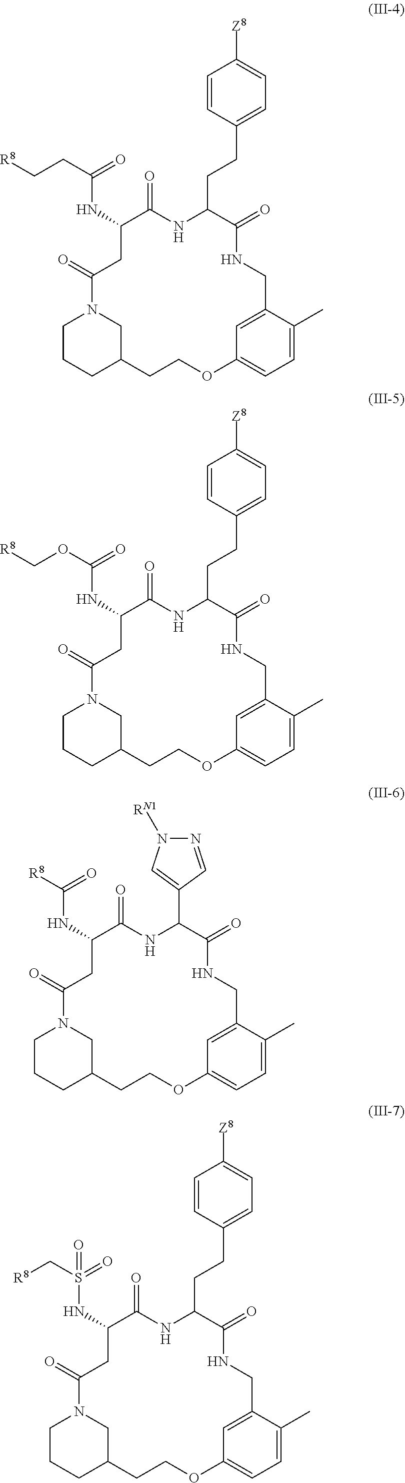

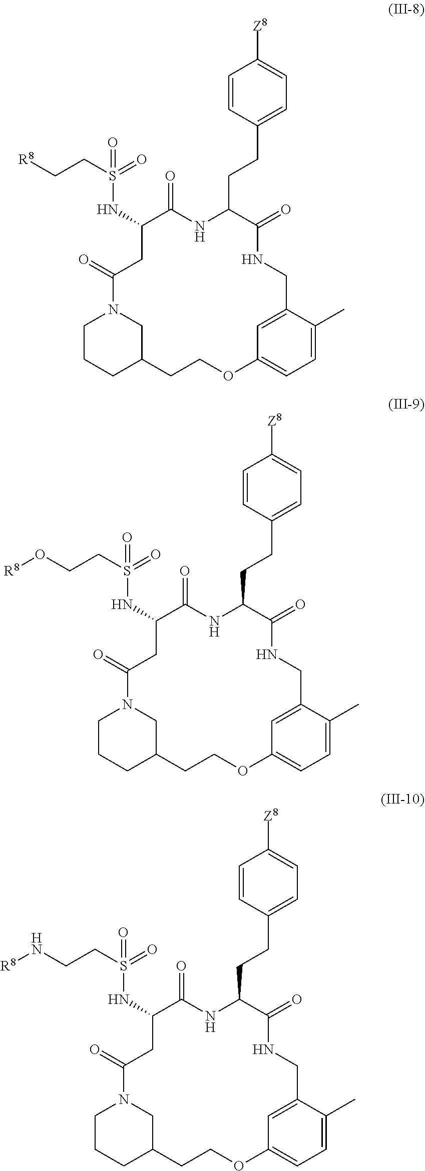

wherein R⁸, R¹², R¹³, R^{N1}, and Z⁸ have the same meanings as defined above.

Preferred are the compounds of any one of the formulae (III-1) to (III-10), wherein R⁸ is

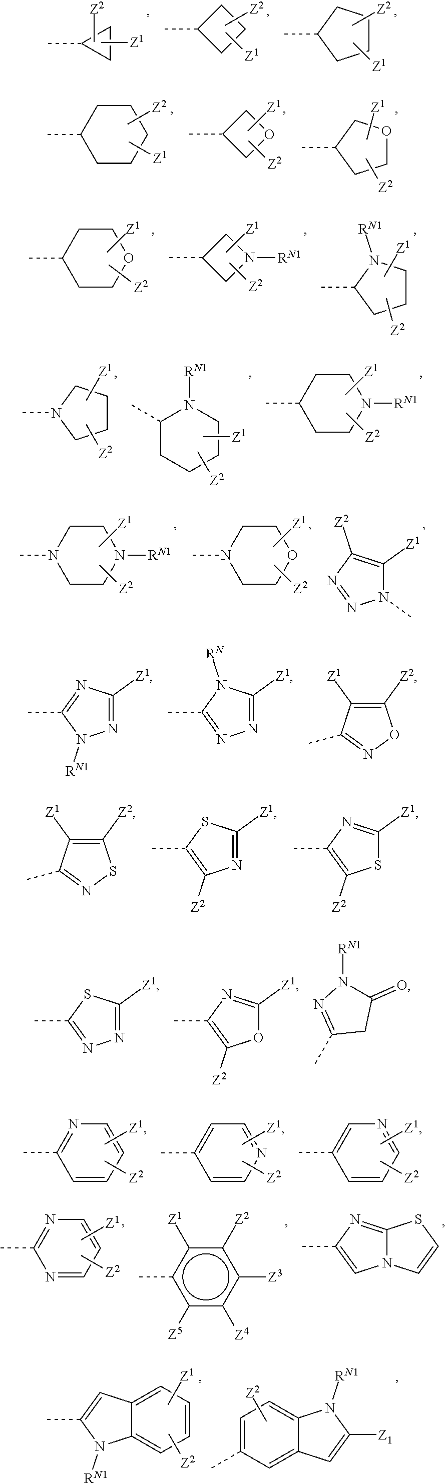

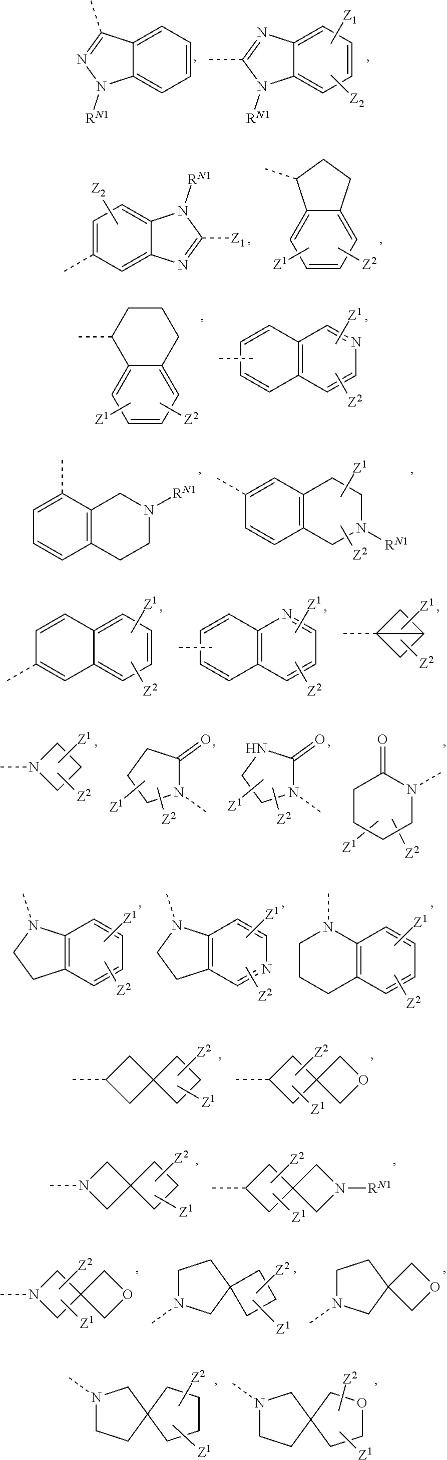

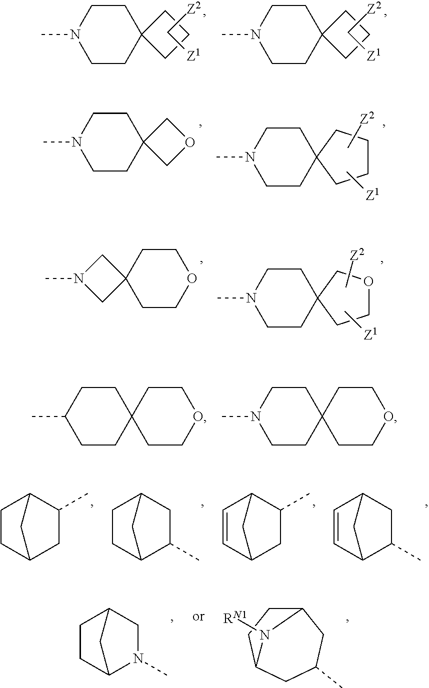

wherein R^{N1}, Z¹, Z², Z³, Z⁴, and Z⁵, have the same meanings as defined in formula (I).

In some embodiments, the present invention relates to the compound having any one of the formulae (IV-1)-(IV-10):

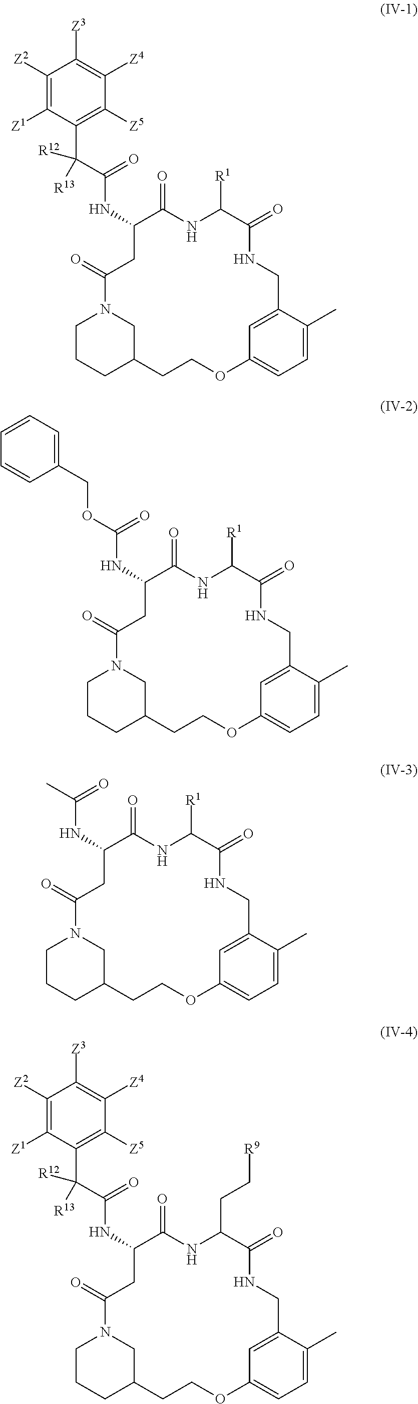

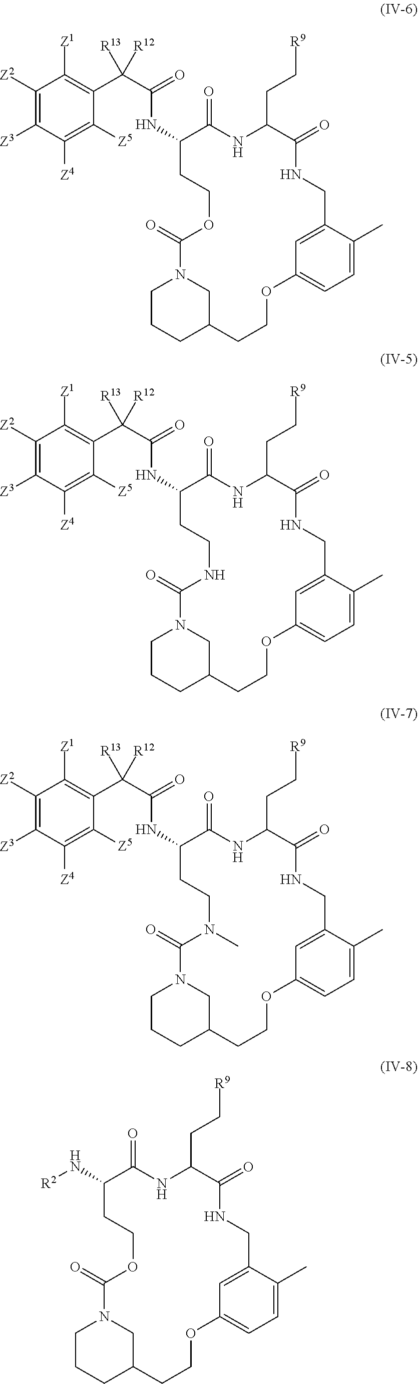

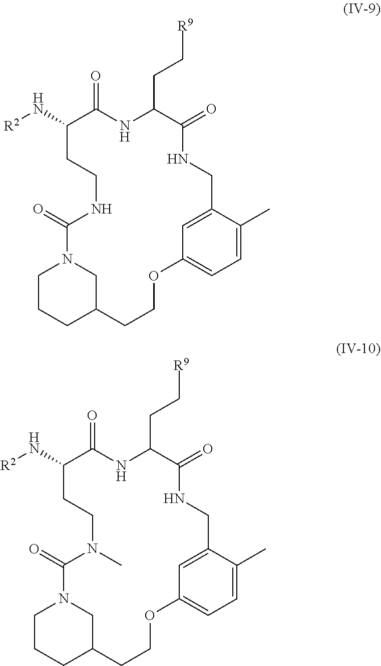

wherein R¹, R², R⁹, R¹², R¹³, Z¹, Z², Z³, Z⁴, and Z⁵ have the same meanings as defined above.

Preferred are the compounds of any one of the formulae (IV-1) to (IV-3), wherein
R¹ represents —(CH₂)ₚ—R⁹, or —(CH₂)ₚ—NR^{N4}—R⁹;
   p is an integer selected from 0, 1, 2, or 3;
   and R⁹ and R^{N4} have the same meaning as defined in the formula (I).

More preferred are the compounds of any one of the formulae (IV-1) to (IV-10), wherein R⁹

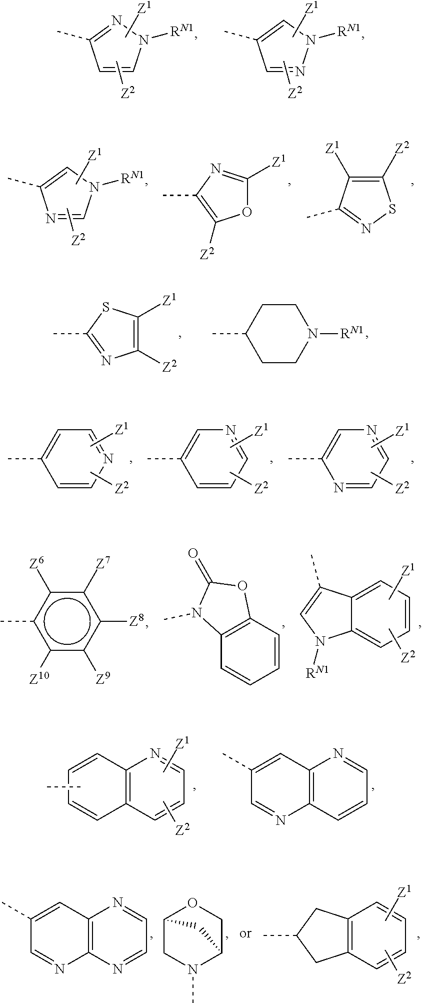

and Z¹, Z², Z⁶, Z⁷, Z⁸, Z⁹, Z¹⁰ and R^{N1} have the same meaning as defined in the formula (I).

In some embodiments, the present invention relates to the compound having any one of the formulae (V-1)-(V-9):

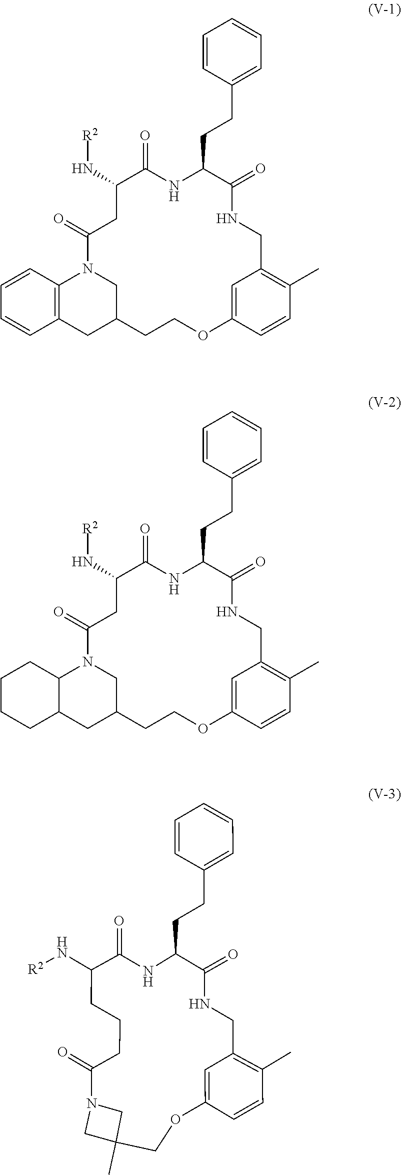

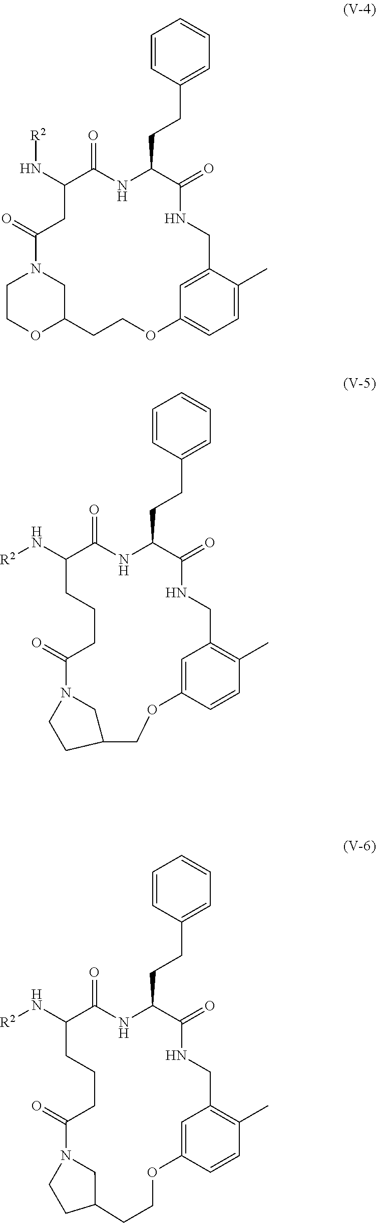

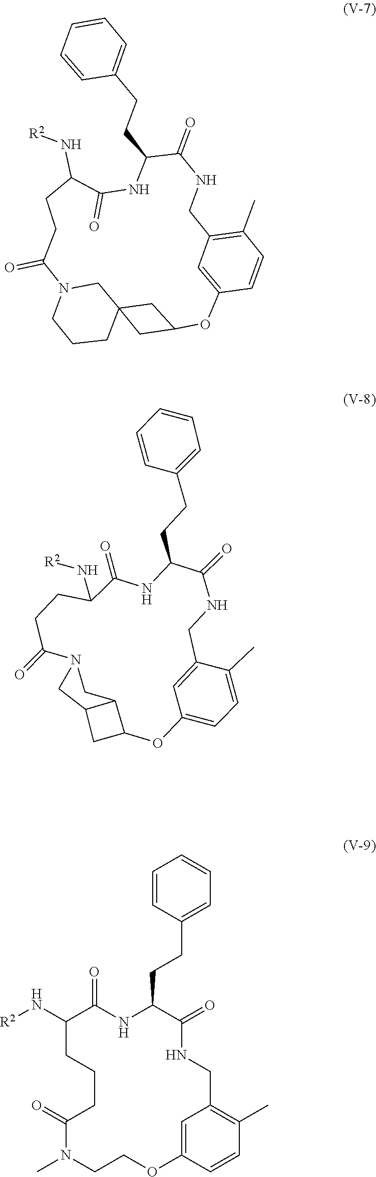

wherein R² has the same meaning as defined above.

Preferred are the compounds of any one of the formulae (V-1) to (V-9), wherein
R² represents —H, -1R⁸, —CO—C(R¹²)(R¹³)—R⁸, -L¹-(CH₂)ₜ—NHR⁸, -L¹-(CH₂)ₜ—NH—CO—R⁸, -L¹-(CH₂)ₜ—NH—SO₂—R⁸, or —CO—C(R¹²)(R¹³)—(CH₂)—R⁸;
R⁸ represents

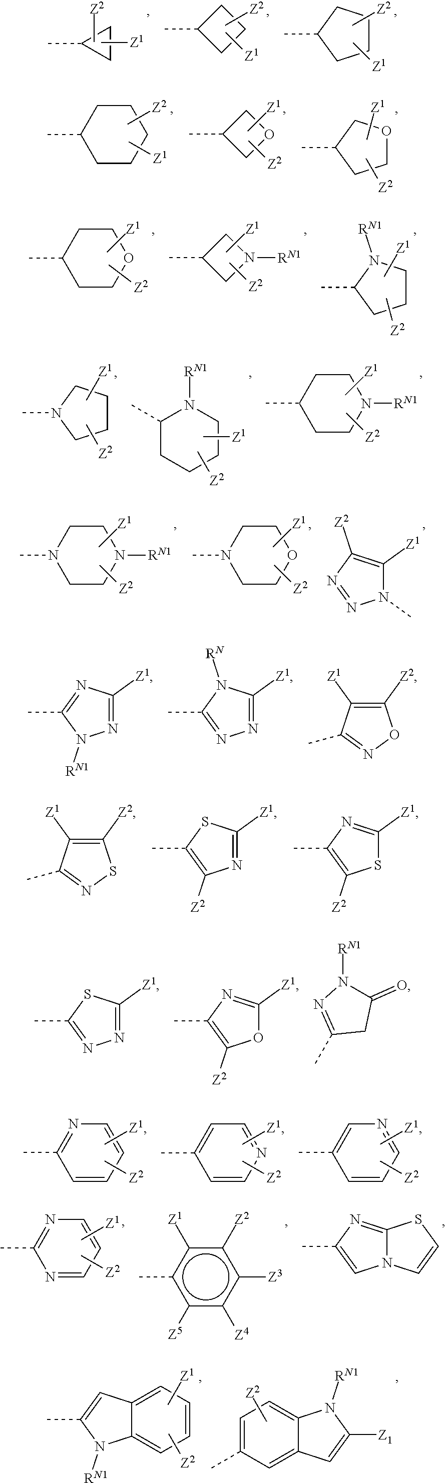

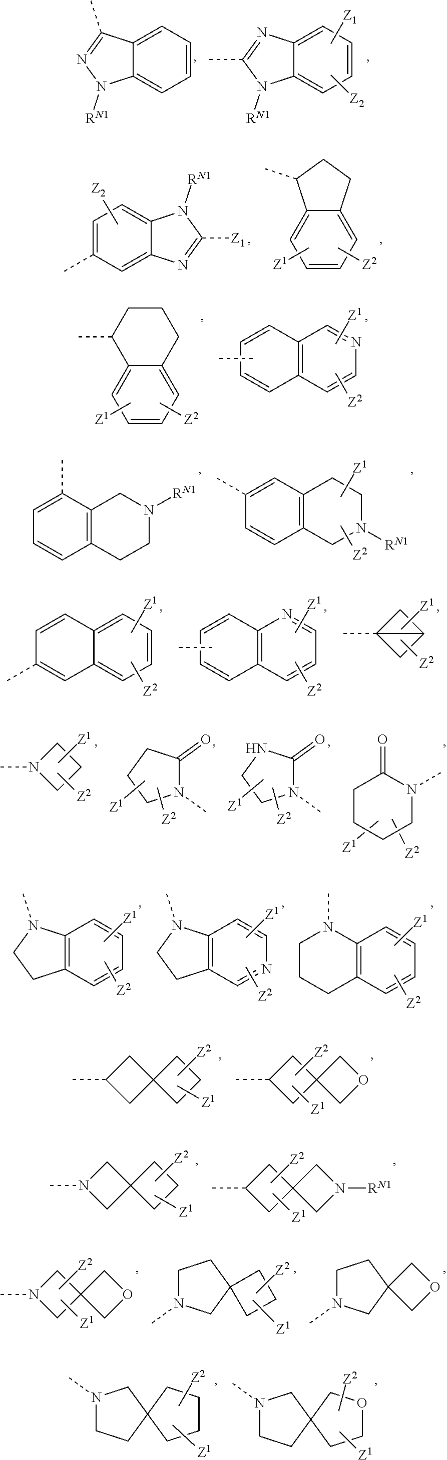

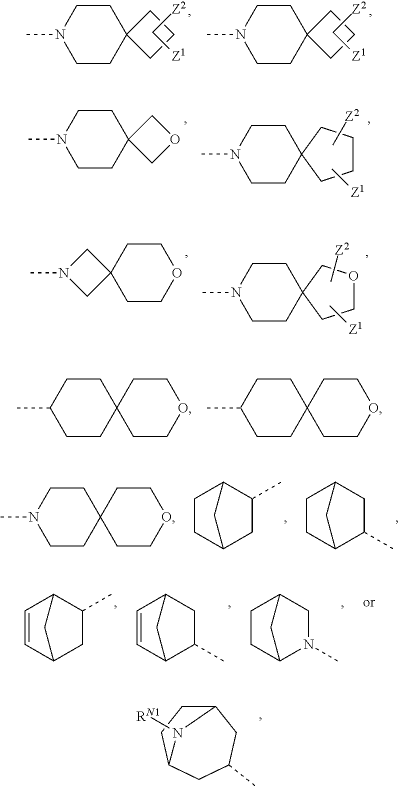

wherein R¹², R¹³, R^{N1}, Z¹, Z², Z⁶, Z⁷, Z⁸, Z⁹, and Z¹⁰, have the same meanings as defined as defined above.

Preferred are the compound of formula (I), (II-1)-(II-16), wherein A represents —CO—NH—, —CO—N(CH₃)—,

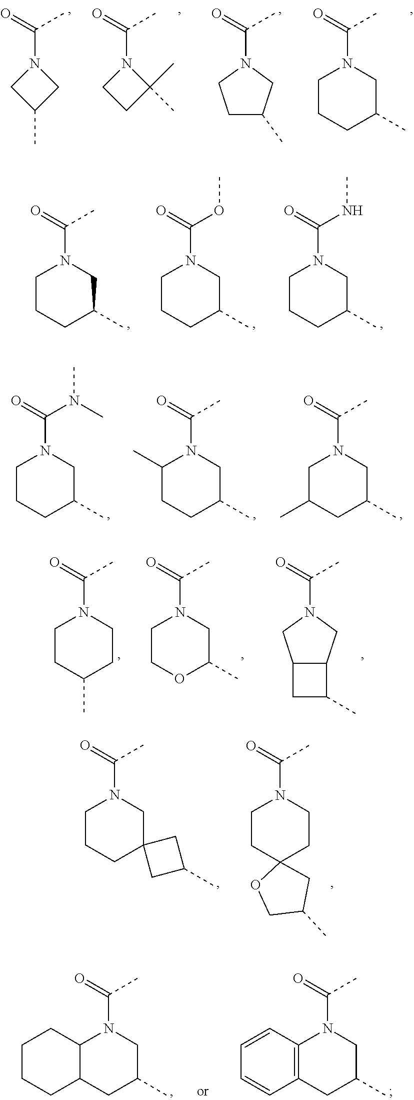

The compounds of any one of the formulae (III-1)-(III-10), (IV-1)-(IV-10), and (V-1)-(V-9) contain also these moieties as A.

Especially preferred compounds according to the present invention include compounds presented by Table 1.

TABLE 1

| Cpd | structure | IUPAC Name |
|---|---|---|
| 7 | 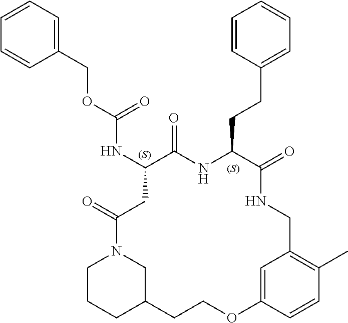 | benzyl ((9S,12S)-5⁴-methyl-8,11,14- trioxo-9-phenethyl-4-oxa-7,10-diaza- 1(3,1)-piperidina-5(1,3)- benzenacyclotetradecaphane-12- yl)carbamate |
| 8 | 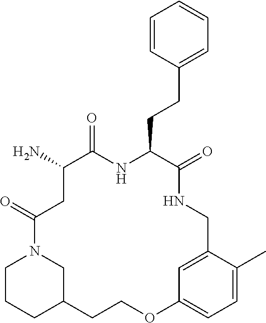 [SMILES: Cc1ccc2cc1CNC(=O)[C@H](CCc1ccccc1)NC(=O)[C@@H](N)CC(=O)N1CCCC(CCO2)C1] | (9S,12S)-12-amino-5⁴-methyl-9- phenethyl-4-oxa-7,10-diaza-1(3,1)- piperidina-5(1,3)- benzenacyclotetradecaphane-8,11,14- trione |
| 13 | 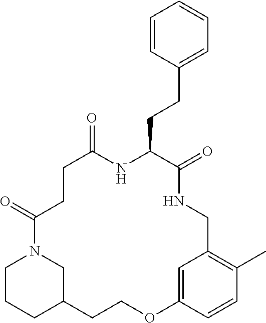 [SMILES: Cc1ccc2cc1CNC(=O)[C@H](CCc1ccccc1)NC(=O)CCC(=O)N1CCCC(CCO2)C1] | (9S)-5⁴-methyl-9-phenethyl-4-oxa-7,10- diaza-1(3,1)-piperidina-5(1,3)- benzenacyclotetradecaphane-8,11,14- trione |
| 16 | 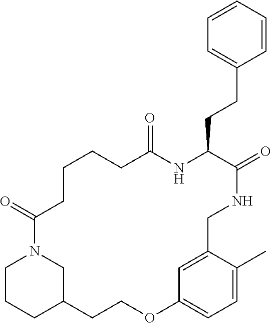 [SMILES: Cc1ccc2cc1CNC(=O)[C@H](CCc1ccccc1)NC(=O)CCCCC(=O)N1CCCC(CCO2)C1] | (9S)-5⁴-methyl-9-phenethyl-4-oxa-7,10- diaza-1(3,1)-piperidina-5(1,3)- benzenacyclohexadecaphane-8,11,16- trione |
| 19 | 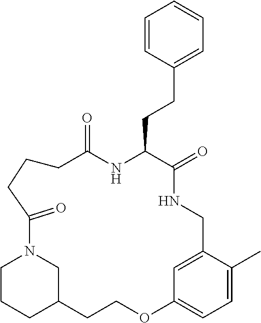 [SMILES: Cc1ccc2cc1CNC(=O)[C@H](CCc1ccccc1)NC(=O)CCCC(=O)N1CCCC(CCO2)C1] | (9S)-5⁴-methyl-9-phenethyl-4-oxa-7,10- diaza-1(3,1)-piperidina-5(1,3)- benzenacyclopentadecaphane-8,11,15- trione |
| 22 | 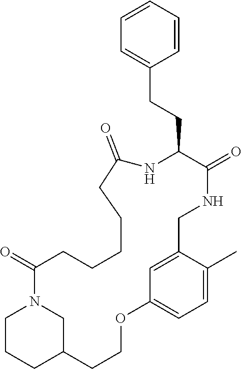 [SMILES: Cc1ccc2cc1CNC(=O)[C@H](CCc1ccccc1)NC(=O)CCCCCC(=O)N1CCCC(CCO2)C1] | (9S)-5⁴-methyl-9-phenethyl-4-oxa-7,10- diaza-1(3,1)-piperidina-5(1,3)- benzenacycloheptadecaphane-8,11,17- trione |
| 25 | 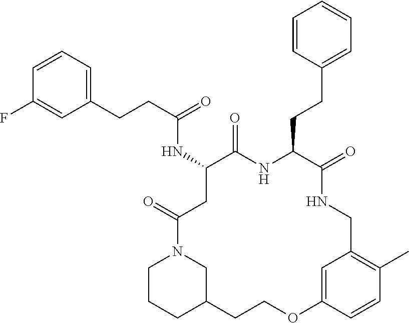 [SMILES: Cc1ccc2cc1CNC(=O)[C@H](CCc1ccccc1)NC(=O)[C@@H](NC(=O)CCc1cccc(F)c1)CC(=O)N1CCCC(CCO2)C1] | 3-(3-fluorophenyl)-N-((9S,12S)-5⁴-methyl- 8,11,14-trioxo-9-phenethyl-4-oxa-7,10- diaza-1(3,1)-piperidina-5(1,3)- benzenacyclotetradecaphane-12- yl)propanamide |
| 26 | 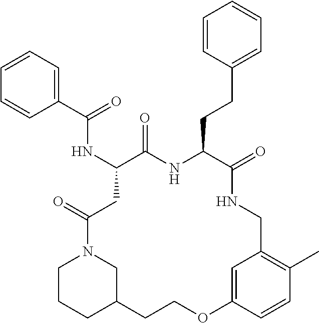 [SMILES: Cc1ccc2cc1CNC(=O)[C@H](CCc1ccccc1)NC(=O)[C@@H](NC(=O)c1ccccc1)CC(=O)N1CCCC(CCO2)C1] | N-((9S,12S)-5⁴-methyl-8,11,14-trioxo-9- phenethyl-4-oxa-7,10-diaza-1(3,1)- piperidina-5(1,3)- benzenacyclotetradecaphane-12- yl)benzamide |
| 27 | 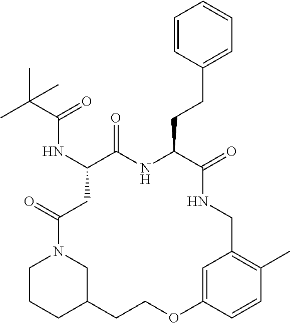 [SMILES: Cc1ccc2cc1CNC(=O)[C@H](CCc1ccccc1)NC(=O)[C@@H](NC(=O)C(C)(C)C)CC(=O)N1CCCC(CCO2)C1] | N-((9S,12S)-5⁴-methyl-8,11,14-trioxo-9- phenethyl-4-oxa-7,10-diaza-1(3,1)- piperidina-5(1,3)- benzenacyclotetradecaphane-12- yl)pivalamide |
| 28 | 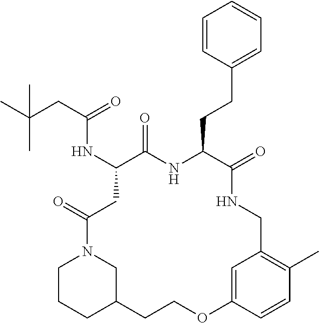 [SMILES: Cc1ccc2cc1CNC(=O)[C@H](CCc1ccccc1)NC(=O)[C@@H](NC(=O)CC(C)(C)C)CC(=O)N1CCCC(CCO2)C1] | 3,3-dimethyl-N-((9S,12S)-5⁴-methyl- 8,11,14-trioxo-9-phenethyl-4-oxa-7,10- diaza-1(3,1)-piperidina-5(1,3)- benzenacyclotetradecaphane-12- yl)butanamide |
| 29 | 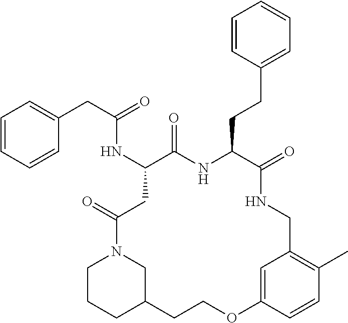 [SMILES: Cc1ccc2cc1CNC(=O)[C@H](CCc1ccccc1)NC(=O)[C@@H](NC(=O)Cc1ccccc1)CC(=O)N1CCCC(CCO2)C1] | N-((9S,12S)-5⁴-methyl-8,11,14-trioxo-9- phenethyl-4-oxa-7,10-diaza-1(3,1)- piperidina-5(1,3)- benzenacyclotetradecaphane-12-yl)-2- phenylacetamide |
| 30 | 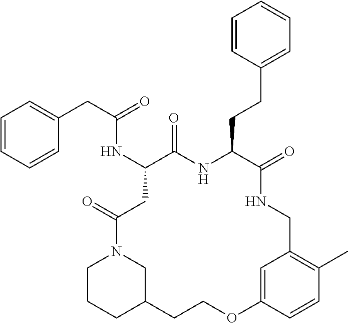 [SMILES: Cc1ccc2cc1CNC(=O)[C@H](CCc1ccccc1)NC(=O)[C@@H](NC(=O)Cc1ccccc1)CC(=O)N1CCCC(CCO2)C1] | N-((9S,12S)-5⁴-methyl-8,11,14-trioxo-9- phenethyl-4-oxa-7,10-diaza-1(3,1)- piperidina-5(1,3)- benzenacyclotetradecaphane-12-yl)-2- phenylacetamide |
| 31 | 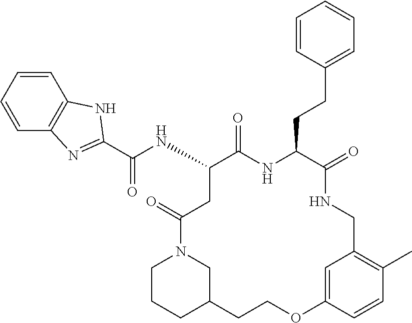 [SMILES: Cc1ccc2cc1CNC(=O)[C@H](CCc1ccccc1)NC(=O)[C@@H](NC(=O)c1nc3ccccc3[nH]1)CC(=O)N1CCCC(CCO2)C1] | N-((9S,12S)-5⁴-methyl-8,11,14-trioxo-9- phenethyl-4-oxa-7,10-diaza-1(3,1)- piperidina-5(1,3)- benzenacyclotetradecaphane-12-yl)-1H- benzo[d]imidazole-2-carboxamide |
| 32 | 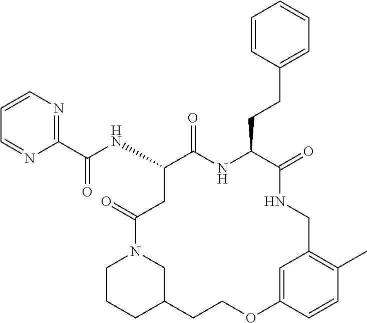 [SMILES: Cc1ccc2cc1CNC(=O)[C@H](CCc1ccccc1)NC(=O)[C@@H](NC(=O)c1ncccn1)CC(=O)N1CCCC(CCO2)C1] | N-((9S,12S)-5⁴-methyl-8,11,14-trioxo-9- phenethyl-4-oxa-7,10-diaza-1(3,1)- piperidina-5(1,3)- benzenacyclotetradecaphane-12- yl)pyrimidine-2-carboxamide |
| 33 | 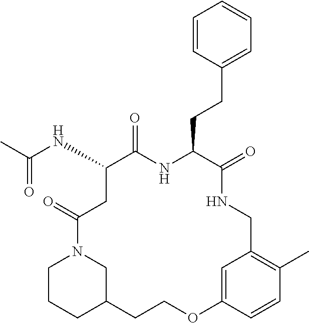 [SMILES: CC(=O)N[C@H]1CC(=O)N2CCCC(CCOc3ccc(C)c(c3)CNC(=O)[C@H](CCc3ccccc3)NC1=O)C2] | N-((9S,12S)-5⁴-methyl-8,11,14-trioxo-9- phenethyl-4-oxa-7,10-diaza-1(3,1)- piperidina-5(1,3)- benzenacyclotetradecaphane-12- yl)acetamide |
| 34 | 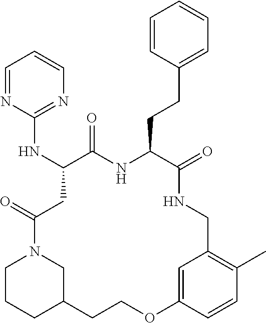 [SMILES: Cc1ccc2cc1CNC(=O)[C@H](CCc1ccccc1)NC(=O)[C@@H](Nc1ncccn1)CC(=O)N1CCCC(CCO2)C1] | (9S,12S)-5⁴-methyl-9-phenethyl-12- (pyrimidin-2-ylamino)-4-oxa-7,10-diaza- 1(3,1)-piperidina-5(1,3)- benzenacyclotetradecaphane-8,11,14- trione |
| 35 | 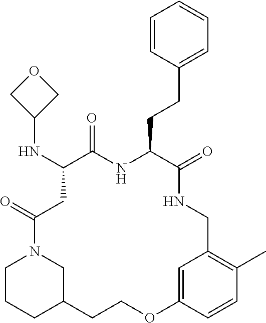 [SMILES: Cc1ccc2cc1CNC(=O)[C@H](CCc1ccccc1)NC(=O)[C@@H](NC1COC1)CC(=O)N1CCCC(CCO2)C1] | (9S,12S)-5⁴-methyl-12-(oxetan-3- ylamino)-9-phenethyl-4-oxa-7,10-diaza- 1(3,1)-piperidina-5(1,3)- benzenacyclotetradecaphane-8,11,14- trione |
| 36 | 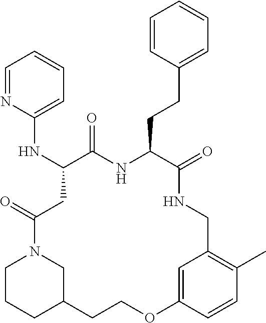 [SMILES: Cc1ccc2cc1CNC(=O)[C@H](CCc1ccccc1)NC(=O)[C@@H](Nc1ccccn1)CC(=O)N1CCCC(CCO2)C1] | (9S,12S)-5⁴-methyl-9-phenethyl-12- (pyridin-2-ylamino)-4-oxa-7,10-diaza- 1(3,1)-piperidina-5(1,3)- benzenacyclotetradecaphane-8,11,14- trione |
| 37 | 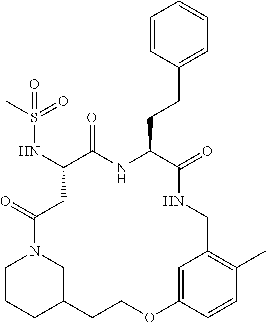 [SMILES: Cc1ccc2cc1CNC(=O)[C@H](CCc1ccccc1)NC(=O)[C@@H](NS(C)(=O)=O)CC(=O)N1CCCC(CCO2)C1] | N-((9S,12S)-5⁴-methyl-8,11,14-trioxo-9- phenethyl-4-oxa-7,10-diaza-1(3,1)- piperidina-5(1,3)- benzenacyclotetradecaphane-12- yl)methanesulfonamide |
| 38 | 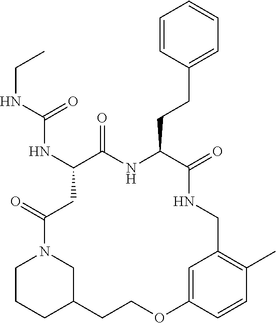 [SMILES: CCNC(=O)N[C@H]1CC(=O)N2CCCC(CCOc3ccc(C)c(c3)CNC(=O)[C@H](CCc3ccccc3)NC1=O)C2] | 1-ethyl-3-((9S,12S)-5⁴-methyl-8,11,14- trioxo-9-phenethyl-4-oxa-7,10-diaza- 1(3,1)-piperidina-5(1,3)- benzenacyclotetradecaphane-12-yl)urea |
| 39 | 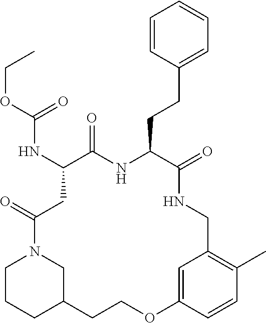 [SMILES: CCOC(=O)N[C@H]1CC(=O)N2CCCC(CCOc3ccc(C)c(c3)CNC(=O)[C@H](CCc3ccccc3)NC1=O)C2] | ethyl ((9S,12S)-5⁴-methyl-8,11,14-trioxo- 9-phenethyl-4-oxa-7,10-diaza-1(3,1)- piperidina-5(1,3)- benzenacyclotetradecaphane-12- yl)carbamate |
| 40 | 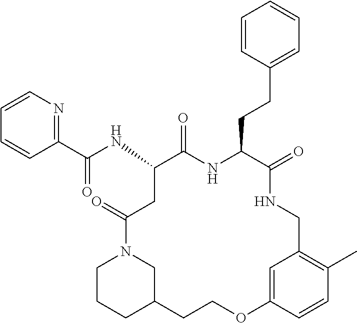 [SMILES: Cc1ccc2cc1CNC(=O)[C@H](CCc1ccccc1)NC(=O)[C@@H](NC(=O)c1ccccn1)CC(=O)N1CCCC(CCO2)C1] | N-((9S,12S)-5⁴-methyl-8,11,14-trioxo-9- phenethyl-4-oxa-7,10-diaza-1(3,1)- piperidina-5(1,3)- benzenacyclotetradecaphane-12- yl)picolinamide |
| 41 | 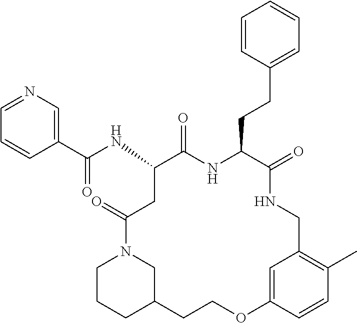 [SMILES: Cc1ccc2cc1CNC(=O)[C@H](CCc1ccccc1)NC(=O)[C@@H](NC(=O)c1cccnc1)CC(=O)N1CCCC(CCO2)C1] | N-((9S,12S)-5⁴-methyl-8,11,14-trioxo-9- phenethyl-4-oxa-7,10-diaza-1(3,1)- piperidina-5(1,3)- benzenacyclotetradecaphane-12- yl)nicotinamide |
| 42 | 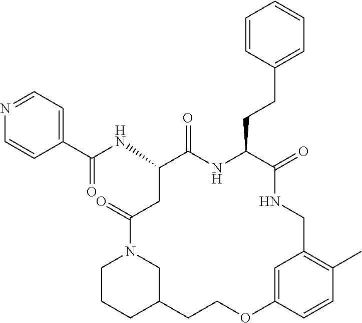 [SMILES: Cc1ccc2cc1CNC(=O)[C@H](CCc1ccccc1)NC(=O)[C@@H](NC(=O)c1ccncc1)CC(=O)N1CCCC(CCO2)C1] | N-((9S,12S)-5⁴-methyl-8,11,14-trioxo-9- phenethyl-4-oxa-7,10-diaza-1(3,1)- piperidina-5(1,3)- benzenacyclotetradecaphane-12- yl)isonicotinamide |
| 43 | 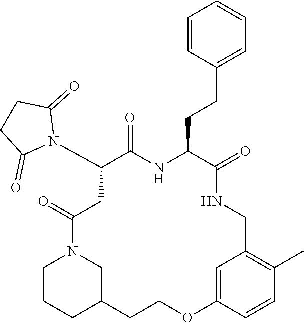 [SMILES: Cc1ccc2cc1CNC(=O)[C@H](CCc1ccccc1)NC(=O)[C@@H](N1C(=O)CCC1=O)CC(=O)N1CCCC(CCO2)C1] | (9S,12S)-12-(2,5-dioxopyrrolidin-1-yl)-5⁴- methyl-9-phenethyl-4-oxa-7,10-diaza- 1(3,1)-piperidina-5(1,3)- benzenacyclotetradecaphane-8,11,14- trione |
| 44 | 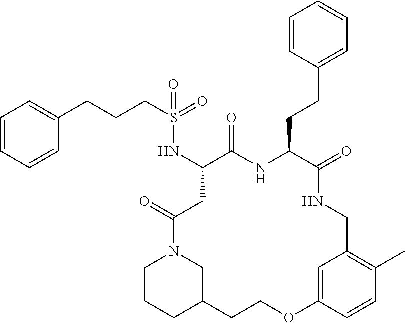 [SMILES: Cc1ccc2cc1CNC(=O)[C@H](CCc1ccccc1)NC(=O)[C@@H](NS(=O)(=O)CCCc1ccccc1)CC(=O)N1CCCC(CCO2)C1] | N-((9S,12S)-5⁴-methyl-8,11,14-trioxo-9- phenethyl-4-oxa-7,10-diaza-1(3,1)- piperidina-5(1,3)- benzenacyclotetradecaphane-12-yl)-3- phenylpropane-1-sulfonamide |
| 45 | 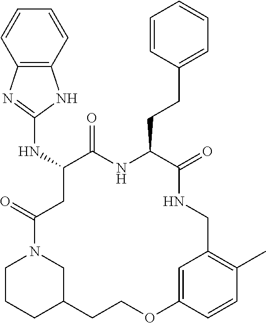 [SMILES: Cc1ccc2cc1CNC(=O)[C@H](CCc1ccccc1)NC(=O)[C@@H](Nc1nc3ccccc3[nH]1)CC(=O)N1CCCC(CCO2)C1] | (9S,12S)-12-((1H-benzo[d]imidazol-2- yl)amino)-5⁴-methyl-9-phenethyl-4-oxa- 7,10-diaza-1(3,1)-piperidina-5(1,3)- benzenacyclotetradecaphane-8,11,14- trione |
| 51 | 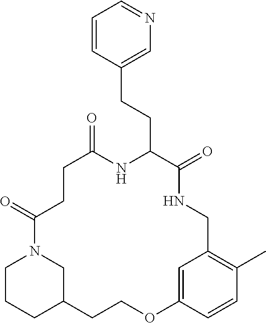 [SMILES: Cc1ccc2cc1CNC(=O)C(CCc1cccnc1)NC(=O)CCC(=O)N1CCCC(CCO2)C1] | 5⁴-methyl-9-(2-(pyridin-3-yl)ethyl)-4-oxa- 7,10-diaza-1(3,1)-piperidina-5(1,3)- benzenacyclotetradecaphane-8,11,14- trione |
| 59 | 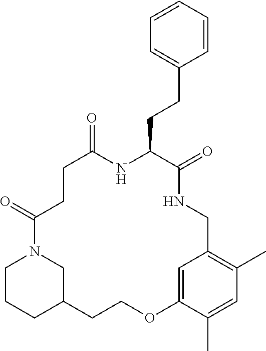 [SMILES: Cc1cc(C)c2cc1CNC(=O)[C@H](CCc1ccccc1)NC(=O)CCC(=O)N1CCCC(CCO2)C1] | (9R)-5⁴,5⁶-dimethyl-9-phenethyl-4-oxa- 7,10-diaza-1(3,1)-piperidina-5(1,3)- benzenacyclotetradecaphane-8,11,14- trione |
| 60 | 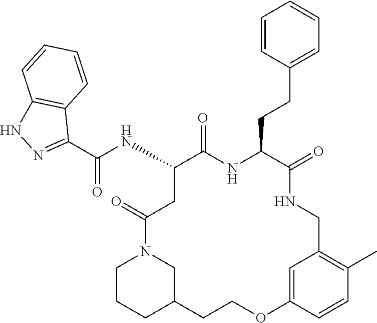 [SMILES: Cc1ccc2cc1CNC(=O)[C@H](CCc1ccccc1)NC(=O)[C@@H](NC(=O)c1n[nH]c3ccccc13)CC(=O)N1CCCC(CCO2)C1] | N-((9S,12S)-5⁴-methyl-8,11,14-trioxo-9- phenethyl-4-oxa-7,10-diaza-1(3,1)- piperidina-5(1,3)- benzenacyclotetradecaphane-12-yl)-1H- indazole-3-carboxamide |
| 61 | 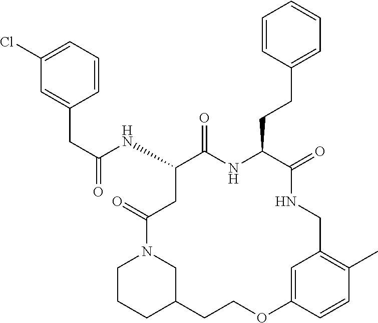 [SMILES: Cc1ccc2cc1CNC(=O)[C@H](CCc1ccccc1)NC(=O)[C@@H](NC(=O)Cc1cccc(Cl)c1)CC(=O)N1CCCC(CCO2)C1] | 2-(3-chlorophenyl)-N-((9S,12S)-5⁴- methyl-8,11,14-trioxo-9-phenethyl-4-oxa- 7,10-diaza-1(3,1)-piperidina-5(1,3)- benzenacyclotetradecaphane-12- yl)acetamide |
| 62 | 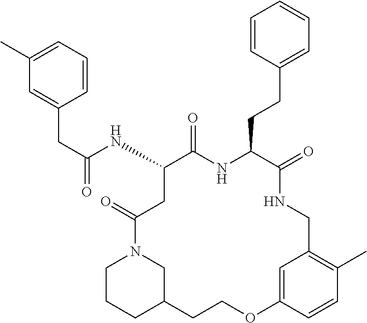 | N-((9S,12S)-5⁴-methyl-8,11,14-trioxo-9- phenethyl-4-oxa-7,10-diaza-1(3,1)- piperidina-5(1,3)- benzenacyclotetradecaphane-12-yl)-2- (m-tolyl)acetamide |
| 63 | 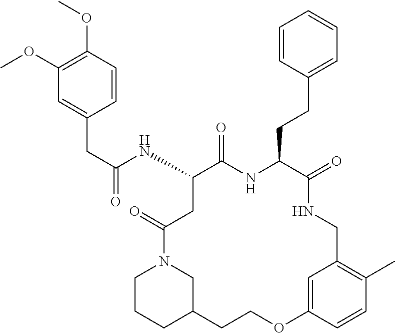 | 2-(3,4-dimethoxyphenyl)-N-((9S,12S)-5⁴- methyl-8,11,14-trioxo-9-phenethyl-4-oxa- 7,10-diaza-1(3,1)-piperidina-5(1,3)- benzenacyclotetradecaphane-12- yl)acetamide |
| 64 | 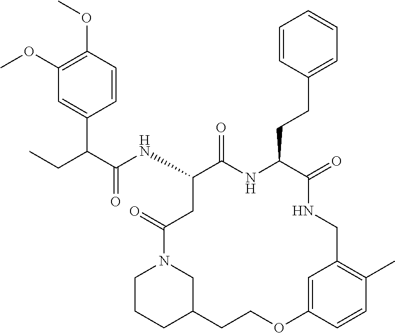 | 2-(3,4-dimethoxyphenyl)-N-((9S,12S)-5⁴- methyl-8,11,14-trioxo-9-phenethyl-4-oxa- 7,10-diaza-1(3,1)-piperidina-5(1,3)- benzenacyclotetradecaphane-12- yl)butanamide |
| 77 | 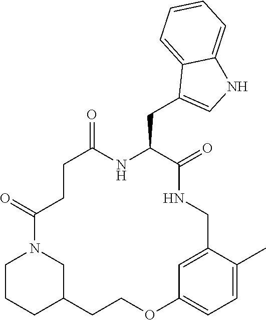 [SMILES: Cc1ccc2cc1CNC(=O)[C@H](Cc1c[nH]c3ccccc13)NC(=O)CCC(=O)N1CCCC(CCO2)C1] | (9S)-9-((1H-indol-3-yl)methyl)-5⁴-methyl- 4-oxa-7,10-diaza-1(3,1)-piperidina-5(1,3)- benzenacyclotetradecaphane-8,11,14- trione |
| 78 | 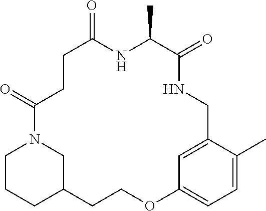 [SMILES: Cc1ccc2cc1CNC(=O)[C@H](C)NC(=O)CCC(=O)N1CCCC(CCO2)C1] | (9S)-5⁴,9-dimethyl-4-oxa-7,10-diaza- 1(3,1)-piperidina-5(1,3)- benzenacyclotetradecaphane-8,11,14- trione |
| 79 | 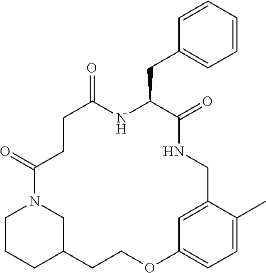 [SMILES: Cc1ccc2cc1CNC(=O)[C@H](Cc1ccccc1)NC(=O)CCC(=O)N1CCCC(CCO2)C1] | (9S)-9-benzyl-5⁴-methyl-4-oxa-7,10- diaza-1(3,1)-piperidina-5(1,3)- benzenacyclotetradecaphane-8,11,14- trione |
| 80 | 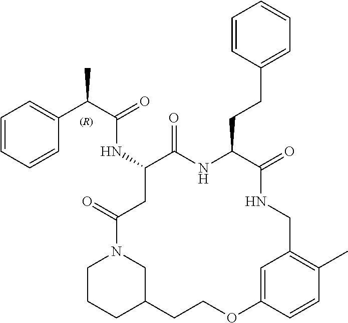 | (2R)-N-((9S,12S)-5⁴-methyl-8,11,14- trioxo-9-phenethyl-4-oxa-7,10-diaza- 1(3,1)-piperidina-5(1,3)- benzenacyclotetradecaphane-12-yl)-2- phenylpropanamide |
| 81 | 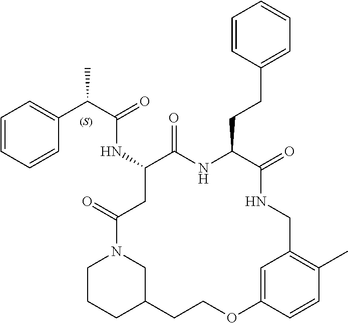 | (2S)-N-((9S,12S)-5⁴-methyl-8,11,14- trioxo-9-phenethyl-4-oxa-7,10-diaza- 1(3,1)-piperidina-5(1,3)- benzenacyclotetradecaphane-12-yl)-2- phenylpropanamide |
| 82 | 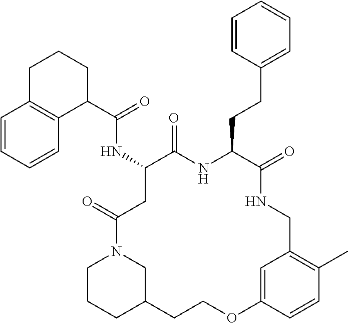 [SMILES: Cc1ccc2cc1CNC(=O)[C@H](CCc1ccccc1)NC(=O)[C@@H](NC(=O)C1CCCc3ccccc31)CC(=O)N1CCCC(CCO2)C1] | N-((9S,12S)-5⁴-methyl-8,11,14-trioxo-9- phenethyl-4-oxa-7,10-diaza-1(3,1)- piperidina-5(1,3)- benzenacyclotetradecaphane-12-yl)- 1,2,3,4-tetrahydronaphthalene-1- carboxamide |
| 83 | 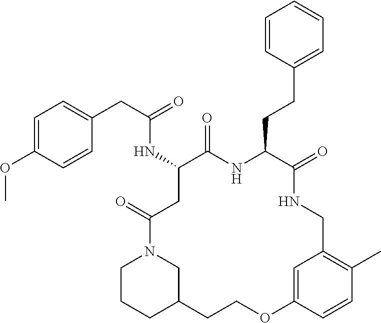 [SMILES: COc1ccc(CC(=O)N[C@H]2CC(=O)N3CCCC(CCOc4ccc(C)c(c4)CNC(=O)[C@H](CCc4ccccc4)NC2=O)C3)cc1] | 2-(4-methoxyphenyl)-N-((9S,12S)-5⁴- methyl-8,11,14-trioxo-9-phenethyl-4-oxa- 7,10-diaza-1(3,1)-piperidina-5(1,3)- benzenacyclotetradecaphane-12- yl)acetamide |
| 84 | 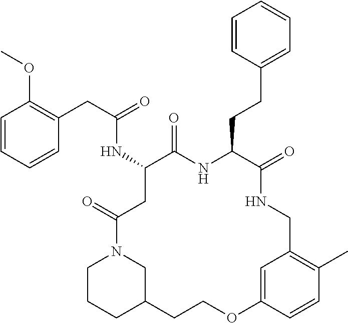 [SMILES: COc1ccccc1CC(=O)N[C@H]1CC(=O)N2CCCC(CCOc3ccc(C)c(c3)CNC(=O)[C@H](CCc3ccccc3)NC1=O)C2] | 2-(2-methoxyphenyl)-N-((9S,12S)-5⁴- methyl-8,11,14-trioxo-9-phenethyl-4-oxa- 7,10-diaza-1(3,1)-piperidina-5(1,3)- benzenacyclotetradecaphane-12- yl)acetamide |
| 85 | 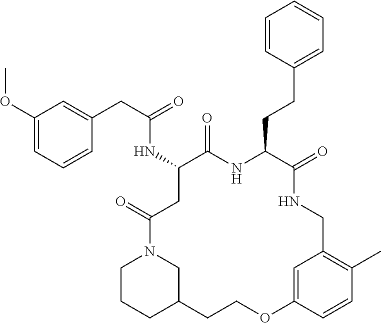 [SMILES: COc1cccc(CC(=O)N[C@H]2CC(=O)N3CCCC(CCOc4ccc(C)c(c4)CNC(=O)[C@H](CCc4ccccc4)NC2=O)C3)c1] | 2-(3-methoxyphenyl)-N-((9S,12S)-5⁴- methyl-8,11,14-trioxo-9-phenethyl-4-oxa- 7,10-diaza-1(3,1)-piperidina-5(1,3)- benzenacyclotetradecaphane-12- yl)acetamide |
| 86 | 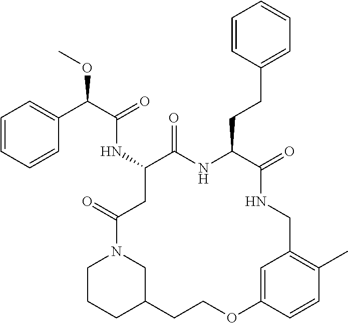 [SMILES: CO[C@@H](C(=O)N[C@H]1CC(=O)N2CCCC(CCOc3ccc(C)c(c3)CNC(=O)[C@H](CCc3ccccc3)NC1=O)C2)c1ccccc1] | (2R)-2-methoxy-N-((9S,12S)-5⁴-methyl- 8,11,14-trioxo-9-phenethyl-4-oxa-7,10- diaza-1(3,1)-piperidina-5(1,3)- benzenacyclotetradecaphane-12-yl)-2- phenylacetamide |
| 87 | 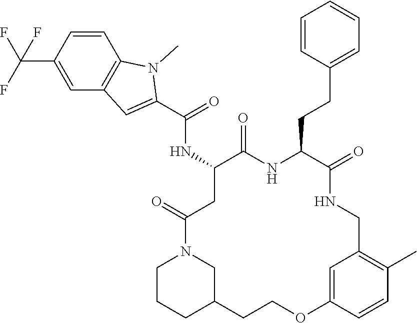 [SMILES: Cc1ccc2cc1CNC(=O)[C@H](CCc1ccccc1)NC(=O)[C@@H](NC(=O)c1cc3cc(C(F)(F)F)ccc3n1C)CC(=O)N1CCCC(CCO2)C1] | 1-methyl-N-((9S,12S)-5⁴-methyl-8,11,14- trioxo-9-phenethyl-4-oxa-7,10-diaza- 1(3,1)-piperidina-5(1,3)- benzenacyclotetradecaphane-12-yl)-5- (trifluoromethyl)-1H-indole-2-carboxamide |
| 88 | 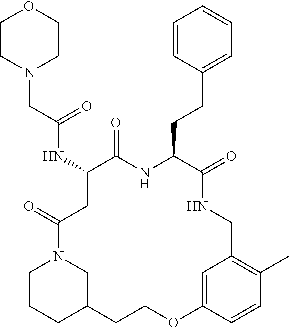 [SMILES: Cc1ccc2cc1CNC(=O)[C@H](CCc1ccccc1)NC(=O)[C@@H](NC(=O)CN1CCOCC1)CC(=O)N1CCCC(CCO2)C1] | N-((9S,12S)-5⁴-methyl-8,11,14-trioxo-9- phenethyl-4-oxa-7,10-diaza-1(3,1)- piperidina-5(1,3)- benzenacyclotetradecaphane-12-yl)-2- morpholinoacetamide |
| 89 | 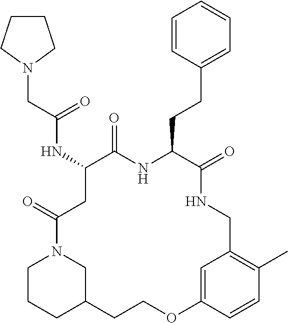 [SMILES: Cc1ccc2cc1CNC(=O)[C@H](CCc1ccccc1)NC(=O)[C@@H](NC(=O)CN1CCCC1)CC(=O)N1CCCC(CCO2)C1] | N-((9S,12S)-5⁴-methyl-8,11,14-trioxo-9- phenethyl-4-oxa-7,10-diaza-1(3,1)- piperidina-5(1,3)- benzenacyclotetradecaphane-12-yl)-2- (pyrrolidin-1-yl)acetamide |
| 95 | 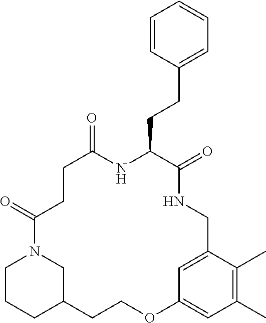 [SMILES: Cc1cc2cc(c1C)CNC(=O)[C@H](CCc1ccccc1)NC(=O)CCC(=O)N1CCCC(CCO2)C1] | (9S)-5⁴,5⁵-dimethyl-9-phenethyl-4-oxa- 7,10-diaza-1(3,1)-piperidina-5(1,3)- benzenacyclotetradecaphane-8,11,14- trione (95) |
| 105 | 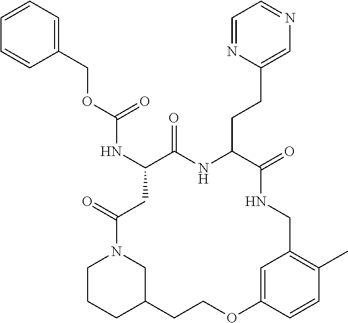 [SMILES: Cc1ccc2cc1CNC(=O)C(CCc1cnccn1)NC(=O)[C@@H](NC(=O)OCc1ccccc1)CC(=O)N1CCCC(CCO2)C1] | benzyl ((12S)-5⁴-methyl-8,11,14-trioxo-9- (2-(pyrazin-2-yl)ethyl)-4-oxa-7,10-diaza- 1(3,1)-piperidina-5(1,3)- benzenacyclotetradecaphane-12- yl)carbamate |
| 106 | 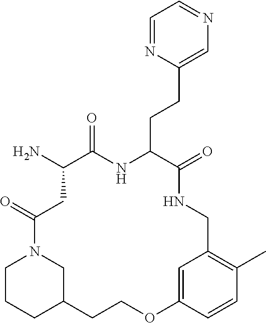 [SMILES: Cc1ccc2cc1CNC(=O)C(CCc1cnccn1)NC(=O)[C@@H](N)CC(=O)N1CCCC(CCO2)C1] | (12S)-amino-5⁴-methyl-8,11,14-trioxo-9- (2-(pyrazin-2-yl)ethyl)-4-oxa-7,10-diaza- 1(3,1)-piperidina-5(1,3)- benzenacyclotetradecaphane |
| 107 | 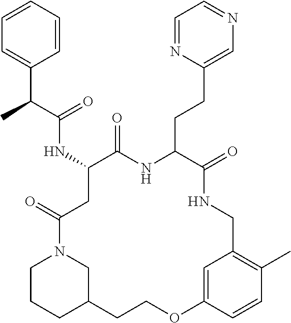 [SMILES: Cc1ccc2cc1CNC(=O)C(CCc1cnccn1)NC(=O)[C@@H](NC(=O)[C@@H](C)c1ccccc1)CC(=O)N1CCCC(CCO2)C1] | (2S)-N-((12S)-5⁴-methyl-8,11,14-trioxo-9- (2-(pyrazin-2-yl)ethyl)-4-oxa-7,10-diaza- 1(3,1)-piperidina-5(1,3)- benzenacyclotetradecaphane-12-yl)-2- phenylpropanamide |
| 116 | 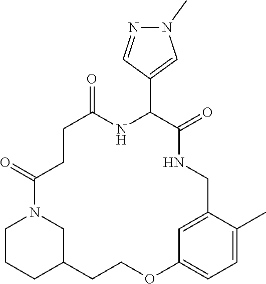 [SMILES: Cc1ccc2cc1CNC(=O)C(c1cnn(C)c1)NC(=O)CCC(=O)N1CCCC(CCO2)C1] | 5⁴-methyl-9-(1-methyl-1H-pyrazol-4-yl)-4- oxa-7,10-diaza-1(3,1)-piperidina-5(1,3)- benzenacyclotetradecaphane-8,11,14- trione |
| 120 | 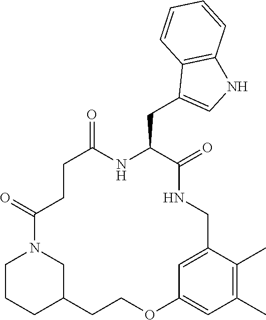 [SMILES: Cc1cc2cc(c1C)CNC(=O)[C@H](Cc1c[nH]c3ccccc13)NC(=O)CCC(=O)N1CCCC(CCO2)C1] | (9S)-9-((1H-indol-3-yl)methyl)-5⁴,5⁵- dimethyl-4-oxa-7,10-diaza-1(3,1)- piperidina-5(1,3)- benzenacyclotetradecaphane-8,11,14- trione |
| 121 | 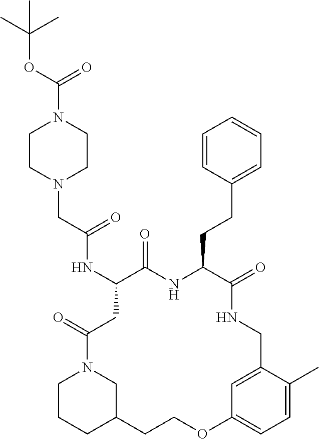 [SMILES: Cc1ccc2cc1CNC(=O)[C@H](CCc1ccccc1)NC(=O)[C@@H](NC(=O)CN1CCN(C(=O)OC(C)(C)C)CC1)CC(=O)N1CCCC(CCO2)C1] | tert-butyl 4-(2-(((9S,12S)-5⁴-methyl- 8,11,14-trioxo-9-phenethyl-4-oxa-7,10- diaza-1(3,1)-piperidina-5(1,3)- benzenacyclotetradecaphane-12- yl)amino)-2-oxoethyl)piperazine-1- carboxylate |
| 122 | 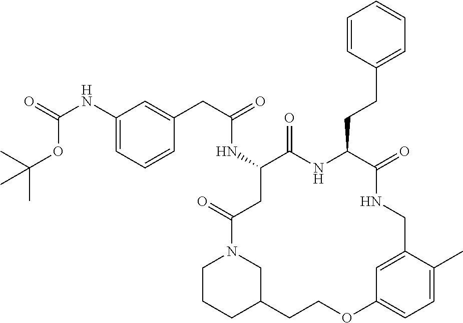 [SMILES: Cc1ccc2cc1CNC(=O)[C@H](CCc1ccccc1)NC(=O)[C@@H](NC(=O)Cc1cccc(NC(=O)OC(C)(C)C)c1)CC(=O)N1CCCC(CCO2)C1] | tert-butyl (3-(2-(((9S,12S)-5⁴-methyl- 8,11,14-trioxo-9-phenethyl-4-oxa-7,10- diaza-1(3,1)-piperidina-5(1,3)- benzenacyclotetradecaphane-12- yl)amino)-2-oxoethyl)phenyl)carbamate |
| 123 | 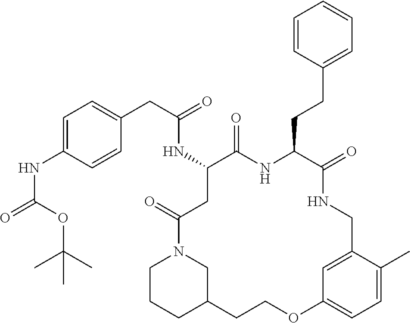 [SMILES: Cc1ccc2cc1CNC(=O)[C@H](CCc1ccccc1)NC(=O)[C@@H](NC(=O)Cc1ccc(NC(=O)OC(C)(C)C)cc1)CC(=O)N1CCCC(CCO2)C1] | tert-butyl (4-(2-(((9S,12S)-5⁴-methyl- 8,11,14-trioxo-9-phenethyl-4-oxa-7,10- diaza-1(3,1)-piperidina-5(1,3)- benzenacyclotetradecaphane-12- yl)amino)-2-oxoethyl)phenyl)carbamate |
| 124 | 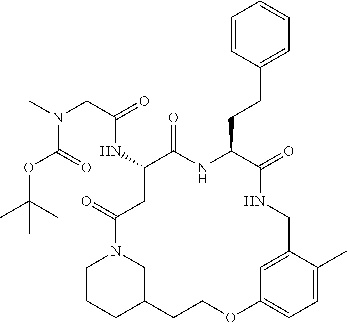 [SMILES: Cc1ccc2cc1CNC(=O)[C@H](CCc1ccccc1)NC(=O)[C@@H](NC(=O)CN(C)C(=O)OC(C)(C)C)CC(=O)N1CCCC(CCO2)C1] | tert-butyl methyl(2-(((9S,12S)-5⁴-methyl- 8,11,14-trioxo-9-phenethyl-4-oxa-7,10- diaza-1(3,1)-piperidina-5(1,3)- benzenacyclotetradecaphane-12- yl)amino)-2-oxoethyl)carbamate |
| 125 | 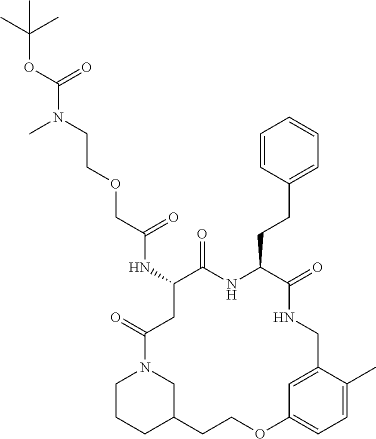 [SMILES: Cc1ccc2cc1CNC(=O)[C@H](CCc1ccccc1)NC(=O)[C@@H](NC(=O)COCCN(C)C(=O)OC(C)(C)C)CC(=O)N1CCCC(CCO2)C1] | tert-butyl methyl(2-(2-(((9S,12S)-5⁴- methyl-8,11,14-trioxo-9-phenethyl-4-oxa- 7,10-diaza-1(3,1)-piperidina-5(1,3)- benzenacyclotetradecaphane-12- yl)amino)-2-oxoethoxy)ethyl)carbamate |
| 126 | 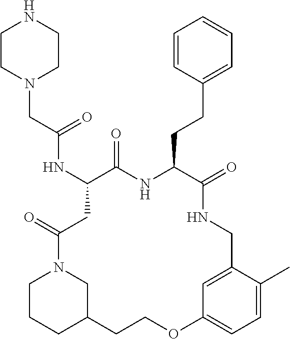 [SMILES: Cc1ccc2cc1CNC(=O)[C@H](CCc1ccccc1)NC(=O)[C@@H](NC(=O)CN1CCNCC1)CC(=O)N1CCCC(CCO2)C1] | N-((9S,12S)-5⁴-methyl-8,11,14-trioxo-9- phenethyl-4-oxa-7,10-diaza-1(3,1)- piperidina-5(1,3)- benzenacyclotetradecaphane-12-yl)-2- (piperazin-1-yl)acetamide |
| 127 | 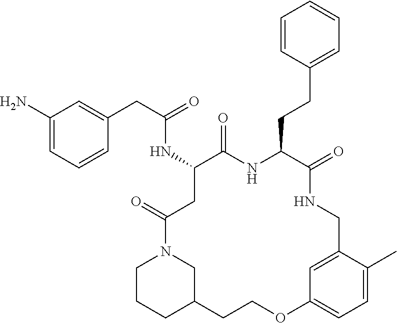 [SMILES: Cc1ccc2cc1CNC(=O)[C@H](CCc1ccccc1)NC(=O)[C@@H](NC(=O)Cc1cccc(N)c1)CC(=O)N1CCCC(CCO2)C1] | 2-(3-aminophenyl)-N-((9S,12S)-5⁴- methyl-8,11,14-trioxo-9-phenethyl-4-oxa- 7,10-diaza-1(3,1)-piperidina-5(1,3)- benzenacyclotetradecaphane-12- yl)acetamide |
| 128 | 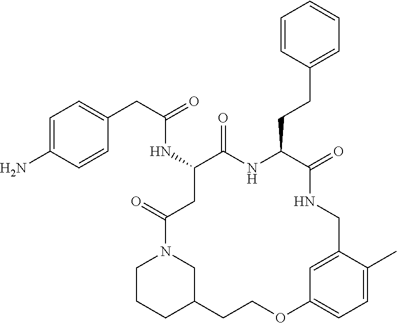 [SMILES: Cc1ccc2cc1CNC(=O)[C@H](CCc1ccccc1)NC(=O)[C@@H](NC(=O)Cc1ccc(N)cc1)CC(=O)N1CCCC(CCO2)C1] | 2-(4-aminophenyl)-N-((9S,12S)-5⁴- methyl-8,11,14-trioxo-9-phenethyl-4-oxa- 7,10-diaza-1(3,1)-piperidina-5(1,3)- benzenacyclotetradecaphane-12- yl)acetamide |
| 129 | 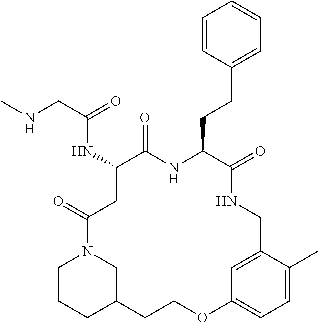 | N-((9S,12S)-5⁴-methyl-8,11,14-trioxo-9- phenethyl-4-oxa-7,10-diaza-1(3,1)- piperidina-5(1,3)- benzenacyclotetradecaphane-12-yl)-2- (methylamino)acetamide |
| 130 | 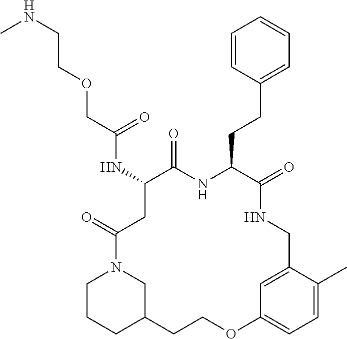 | N-((9S,12S)-5⁴-methyl-8,11,14-trioxo-9- phenethyl-4-oxa-7,10-diaza-1(3,1)- piperidina-5(1,3)- benzenacyclotetradecaphane-12-yl)-2-(2- (methylamino)ethoxy)acetamide |
| 131 | 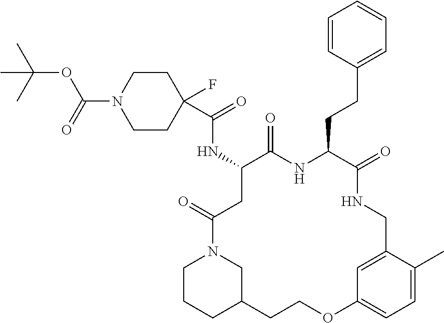 [SMILES: Cc1ccc2cc1CNC(=O)[C@H](CCc1ccccc1)NC(=O)[C@@H](NC(=O)C1(F)CCN(C(=O)OC(C)(C)C)CC1)CC(=O)N1CCCC(CCO2)C1] | tert-butyl 4-fluoro-4-(((9S,12S)-5⁴-methyl- 8,11,14-trioxo-9-phenethyl-4-oxa-7,10- diaza-1(3,1)-piperidina-5(1,3)- benzenacyclotetradecaphane-12- yl)carbamoyl)piperidine-1-carboxylate |
| 132 | 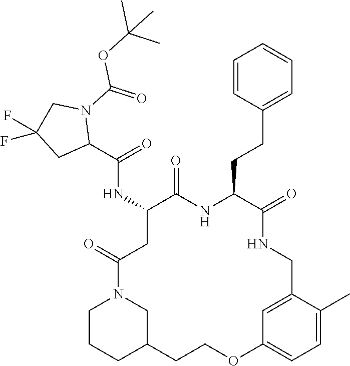 [SMILES: Cc1ccc2cc1CNC(=O)[C@H](CCc1ccccc1)NC(=O)[C@@H](NC(=O)C1CC(F)(F)CN1C(=O)OC(C)(C)C)CC(=O)N1CCCC(CCO2)C1] | tert-butyl 4,4-difluoro-2-(((9S,12S)-5⁴- methyl-8,11,14-trioxo-9-phenethyl-4-oxa- 7,10-diaza-1(3,1)-piperidina-5(1,3)- benzenacyclotetradecaphane-12- yl)carbamoyl)pyrrolidine-1-carboxylate |
| 133 | 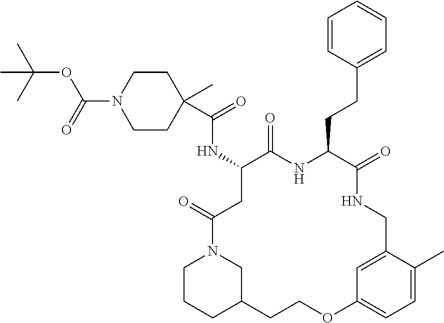 [SMILES: Cc1ccc2cc1CNC(=O)[C@H](CCc1ccccc1)NC(=O)[C@@H](NC(=O)C1(C)CCN(C(=O)OC(C)(C)C)CC1)CC(=O)N1CCCC(CCO2)C1] | tert-butyl 4-methyl-4-(((9S,12S)-5⁴- methyl-8,11,14-trioxo-9-phenethyl-4-oxa- 7,10-diaza-1(3,1)-piperidina-5(1,3)- benzenacyclotetradecaphane-12- yl)carbamoyl)piperidine-1-carboxylate |
| 134 | 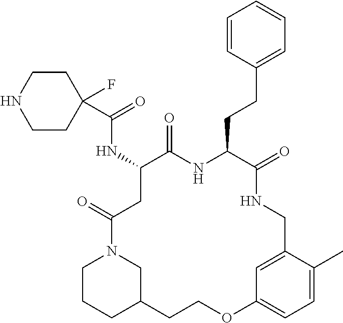 [SMILES: Cc1ccc2cc1CNC(=O)[C@H](CCc1ccccc1)NC(=O)[C@@H](NC(=O)C1(F)CCNCC1)CC(=O)N1CCCC(CCO2)C1] | 4-fluoro-N-((9S,12S)-5⁴-methyl-8,11,14- trioxo-9-phenethyl-4-oxa-7,10-diaza- 1(3,1)-piperidina-5(1,3)- benzenacyclotetradecaphane-12- yl)piperidine-4-carboxamide |
| 135 | 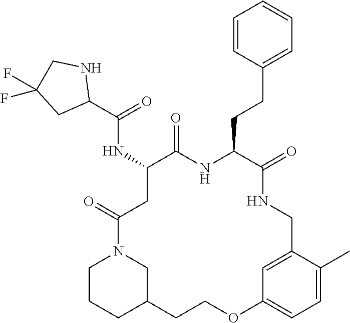 [SMILES: Cc1ccc2cc1CNC(=O)[C@H](CCc1ccccc1)NC(=O)[C@@H](NC(=O)C1CC(F)(F)CN1)CC(=O)N1CCCC(CCO2)C1] | (2S)-4,4-difluoro-N-((9S,12S)-5⁴-methyl- 8,11,14-trioxo-9-phenethyl-4-oxa-7,10- diaza-1(3,1)-piperidina-5(1,3)- benzenacyclotetradecaphane-12- yl)pyrrolidine-2-carboxamide |
| 136 | 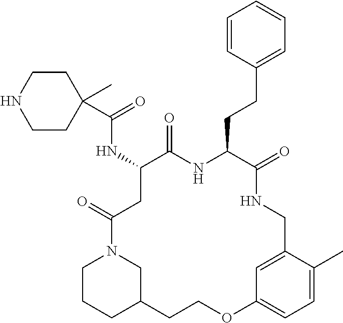 [SMILES: Cc1ccc2cc1CNC(=O)[C@H](CCc1ccccc1)NC(=O)[C@@H](NC(=O)C1(C)CCNCC1)CC(=O)N1CCCC(CCO2)C1] | 4-methyl-N-((9S,12S)-5⁴-methyl-8,11,14- trioxo-9-phenethyl-4-oxa-7,10-diaza- 1(3,1)-piperidina-5(1,3)- benzenacyclotetradecaphane-12- yl)piperidine-4-carboxamide |
| 143 | 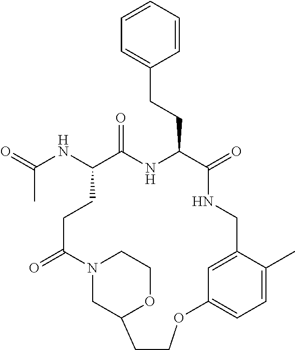 [SMILES: CC(=O)N[C@H]1CCC(=O)N2CCOC(CCOc3ccc(C)c(c3)CNC(=O)[C@H](CCc3ccccc3)NC1=O)C2] | N-((9S,12S)-5⁴-methyl-8,11,15-trioxo-9- phenethyl-4-oxa-7,10-diaza-1(2,4)- morpholina-5(1,3)- benzenacyclopentadecaphane-12- yl)acetamide |
| 148 | 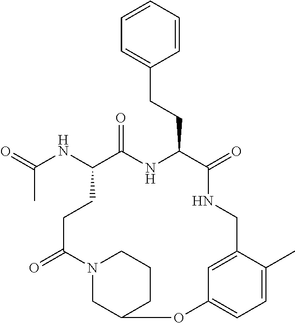 [SMILES: CC(=O)N[C@H]1CCC(=O)N2CCCC(C2)Oc2ccc(C)c(c2)CNC(=O)[C@H](CCc2ccccc2)NC1=O] | N-((7S,10S)-3⁴-methyl-6,9,13-trioxo-7- phenethyl-2-oxa-5,8-diaza-1(3,1)- piperidina-3(1,3)- benzenacyclotridecaphane-10- yl)acetamide |
| 149 | 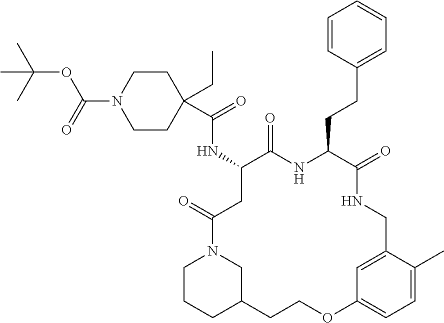 [SMILES: CCC1(C(=O)N[C@H]2CC(=O)N3CCCC(CCOc4ccc(C)c(c4)CNC(=O)[C@H](CCc4ccccc4)NC2=O)C3)CCN(C(=O)OC(C)(C)C)CC1] | tert-butyl 4-ethyl-4-(((9S,12S)-5⁴-methyl- 8,11,14-trioxo-9-phenethyl-4-oxa-7,10- diaza-1(3,1)-piperidina-5(1,3)- benzenacyclotetradecaphane-12- yl)carbamoyl)piperidine-1-carboxylate |
| 150 | 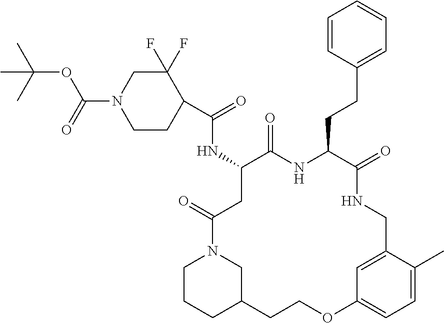 [SMILES: Cc1ccc2cc1CNC(=O)[C@H](CCc1ccccc1)NC(=O)[C@@H](NC(=O)C1CCN(C(=O)OC(C)(C)C)CC1(F)F)CC(=O)N1CCCC(CCO2)C1] | tert-butyl 3,3-difluoro-4-(((9S,12S)-5⁴- methyl-8,11,14-trioxo-9-phenethyl-4-oxa- 7,10-diaza-1(3,1)-piperidina-5(1,3)- benzenacyclotetradecaphane-12- yl)carbamoyl)piperidine-1-carboxylate |
| 151 | 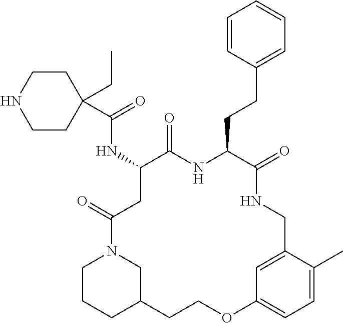 [SMILES: CCC1(C(=O)N[C@H]2CC(=O)N3CCCC(CCOc4ccc(C)c(c4)CNC(=O)[C@H](CCc4ccccc4)NC2=O)C3)CCNCC1] | 4-ethyl-N-((9S,12S)-5⁴-methyl-8,11,14- trioxo-9-phenethyl-4-oxa-7,10-diaza- 1(3,1)-piperidina-5(1,3)- benzenacyclotetradecaphane-12- yl)piperidine-4-carboxamide |
| 152 | 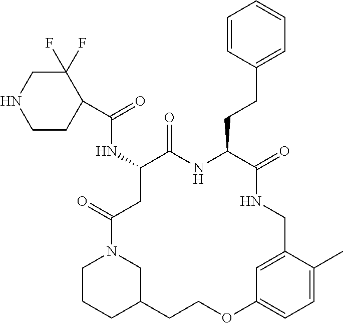 [SMILES: Cc1ccc2cc1CNC(=O)[C@H](CCc1ccccc1)NC(=O)[C@@H](NC(=O)C1CCNCC1(F)F)CC(=O)N1CCCC(CCO2)C1] | 3,3-difluoro-N-((9S,12S)-5⁴-methyl- 8,11,14-trioxo-9-phenethyl-4-oxa-7,10- diaza-1(3,1)-piperidina-5(1,3)- benzenacyclotetradecaphane-12- yl)piperidine-4-carboxamide |
| 153 | 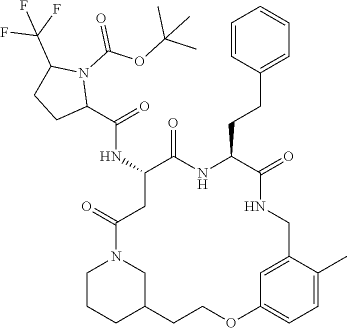 [SMILES: Cc1ccc2cc1CNC(=O)[C@H](CCc1ccccc1)NC(=O)[C@@H](NC(=O)C1CCC(C(F)(F)F)N1C(=O)OC(C)(C)C)CC(=O)N1CCCC(CCO2)C1] | tert-butyl 2-(((9S,12S)-5⁴-methyl-8,11,14- trioxo-9-phenethyl-4-oxa-7,10-diaza- 1(3,1)-piperidina-5(1,3)- benzenacyclotetradecaphane-12- yl)carbamoyl)-5- (trifluoromethyl)pyrrolidine-1-carboxylate |
| 154 | 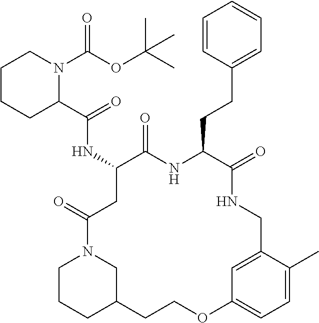 [SMILES: Cc1ccc2cc1CNC(=O)[C@H](CCc1ccccc1)NC(=O)[C@@H](NC(=O)C1CCCCN1C(=O)OC(C)(C)C)CC(=O)N1CCCC(CCO2)C1] | tert-butyl 2-(((9S,12S)-5⁴-methyl-8,11,14- trioxo-9-phenethyl-4-oxa-7,10-diaza- 1(3,1)-piperidina-5(1,3)- benzenacyclotetradecaphane-12- yl)carbamoyl)piperidine-1-carboxylate |
| 155 | 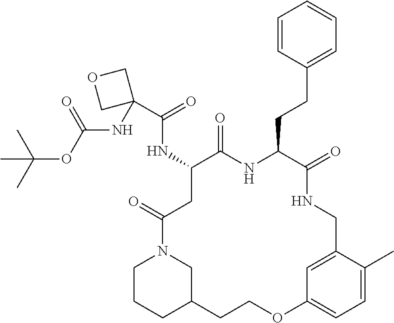 [SMILES: Cc1ccc2cc1CNC(=O)[C@H](CCc1ccccc1)NC(=O)[C@@H](NC(=O)C1(NC(=O)OC(C)(C)C)COC1)CC(=O)N1CCCC(CCO2)C1] | tert-butyl (3-(((9S,12S)-5⁴-methyl-8,11,14- trioxo-9-phenethyl-4-oxa-7,10-diaza- 1(3,1)-piperidina-5(1,3)- benzenacyclotetradecaphane-12- yl)carbamoyl)oxetan-3-yl)carbamate |
| 156 | 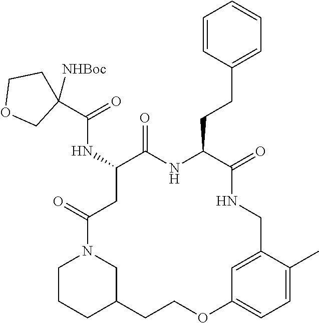 [SMILES: Cc1ccc2cc1CNC(=O)[C@H](CCc1ccccc1)NC(=O)[C@@H](NC(=O)C1(NC(=O)OC(C)(C)C)CCOC1)CC(=O)N1CCCC(CCO2)C1] | tert-butyl (3-(((9S,12S)-5⁴-methyl-8,11,14- trioxo-9-phenethyl-4-oxa-7,10-diaza- 1(3,1)-piperidina-5(1,3)- benzenacyclotetradecaphane-12- yl)carbamoyl)tetrahydrofuran-3- yl)carbamate |
| 157 | 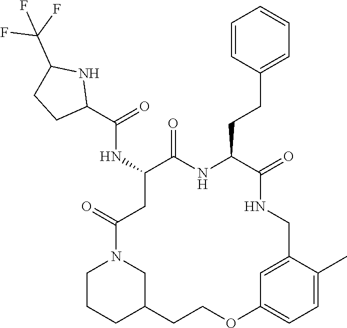 [SMILES: Cc1ccc2cc1CNC(=O)[C@H](CCc1ccccc1)NC(=O)[C@@H](NC(=O)C1CCC(C(F)(F)F)N1)CC(=O)N1CCCC(CCO2)C1] | N-((9S,12S)-5⁴-methyl-8,11,14-trioxo-9- phenethyl-4-oxa-7,10-diaza-1(3,1)- piperidina-5(1,3)- benzenacyclotetradecaphane-12-yl)-5- (trifluoromethyl)pyrrolidine-2-carboxamide |
| 158 | 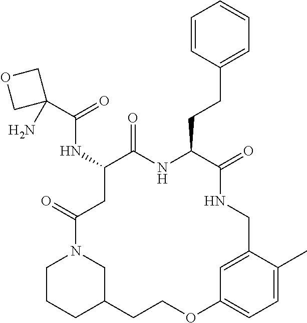 [SMILES: Cc1ccc2cc1CNC(=O)[C@H](CCc1ccccc1)NC(=O)[C@@H](NC(=O)C1(N)COC1)CC(=O)N1CCCC(CCO2)C1] | 3-amino-N-((9S,12S)-5⁴-methyl-8,11,14- trioxo-9-phenethyl-4-oxa-7,10-diaza- 1(3,1)-piperidina-5(1,3)- benzenacyclotetradecaphane-12- yl)oxetane-3-carboxamide |
| 159 | 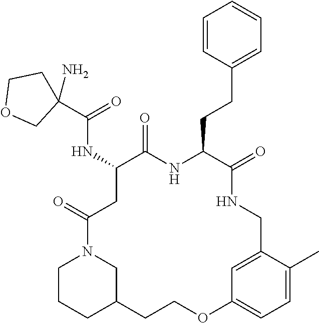 [SMILES: Cc1ccc2cc1CNC(=O)[C@H](CCc1ccccc1)NC(=O)[C@@H](NC(=O)C1(N)CCOC1)CC(=O)N1CCCC(CCO2)C1] | 3-amino-N-((9S,12S)-5⁴-methyl-8,11,14- trioxo-9-phenethyl-4-oxa-7,10-diaza- 1(3,1)-piperidina-5(1,3)- benzenacyclotetradecaphane-12- yl)tetrahydrofuran-3-carboxamide |
| 160 | 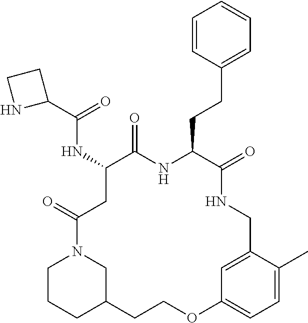 [SMILES: Cc1ccc2cc1CNC(=O)[C@H](CCc1ccccc1)NC(=O)[C@@H](NC(=O)C1CCN1)CC(=O)N1CCCC(CCO2)C1] | (2R)-N-((9S,12S)-5⁴-methyl-8,11,14- trioxo-9-phenethyl-4-oxa-7,10-diaza- 1(3,1)-piperidina-5(1,3)- benzenacyclotetradecaphane-12- yl)azetidine-2-carboxamide |
| 161 | 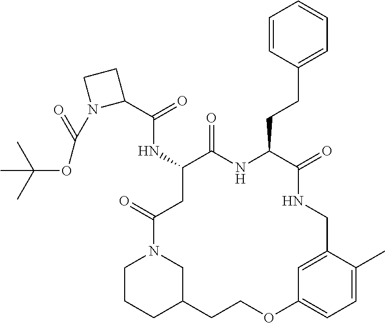 [SMILES: Cc1ccc2cc1CNC(=O)[C@H](CCc1ccccc1)NC(=O)[C@@H](NC(=O)C1CCN1C(=O)OC(C)(C)C)CC(=O)N1CCCC(CCO2)C1] | tert-butyl 2-(((9S,12S)-5⁴-methyl-8,11,14- trioxo-9-phenethyl-4-oxa-7,10-diaza- 1(3,1)-piperidina-5(1,3)- benzenacyclotetradecaphane-12- yl)carbamoyl)azetidine-1-carboxylate |
| 168 | 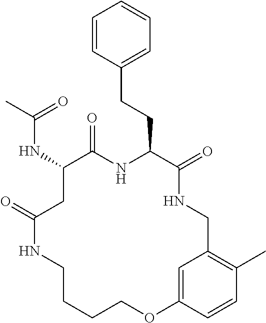 [SMILES: CC(=O)N[C@H]1CC(=O)NCCCCOc2ccc(C)c(c2)CNC(=O)[C@H](CCc2ccccc2)NC1=O] | N-((10S,13S)-1⁴-methyl-8,11,14-trioxo- 13-phenethyl-2-oxa-7,12,15-triaza-1(1,3)- benzenacyclohexadecaphane-10- yl)acetamide |
| 171 | 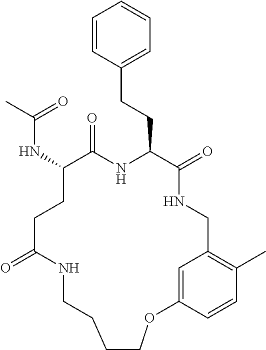 [SMILES: CC(=O)N[C@H]1CCC(=O)NCCCCOc2ccc(C)c(c2)CNC(=O)[C@H](CCc2ccccc2)NC1=O] | N-((11S,14S)-1⁴-methyl-8,12,15-trioxo- 14-phenethyl-2-oxa-7,13,16-triaza-1(1,3)- benzenacycloheptadecaphane-11- yl)acetamide |
| 172 | 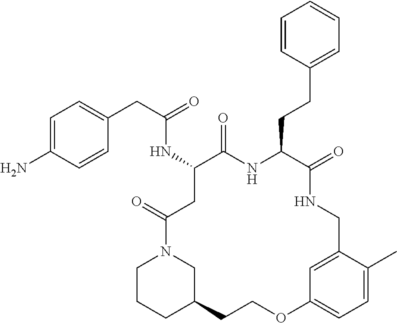 [SMILES: Cc1ccc2cc1CNC(=O)[C@H](CCc1ccccc1)NC(=O)[C@@H](NC(=O)Cc1ccc(N)cc1)CC(=O)N1CCC[C@H](CCO2)C1] | 2-(4-aminophenyl)-N-((13R,9S,12S)-5⁴- methyl-8,11,14-trioxo-9-phenethyl-4-oxa- 7,10-diaza-1(3,1)-piperidina-5(1,3)- benzenacyclotetradecaphane-12- yl)acetamide |
| 173 | 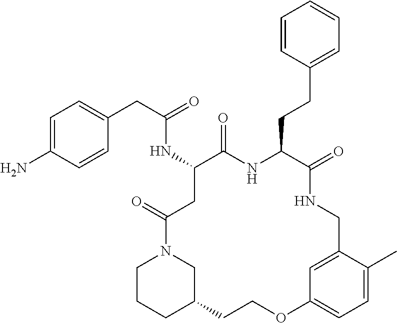 [SMILES: Cc1ccc2cc1CNC(=O)[C@H](CCc1ccccc1)NC(=O)[C@@H](NC(=O)Cc1ccc(N)cc1)CC(=O)N1CCC[C@@H](CCO2)C1] | 2-(4-aminophenyl)-N-((13S,9S,12S)-5⁴- methyl-8,11,14-trioxo-9-phenethyl-4-oxa- 7,10-diaza-1(3,1)-piperidina-5(1,3)- benzenacyclotetradecaphane-12- yl)acetamide |
| 174 | 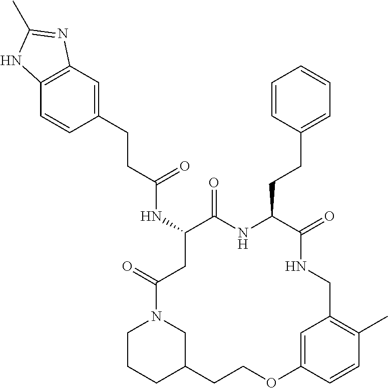 | 3-(2-methyl-1H-benzo[d]imidazol-6-yl)-N- ((9S,12S)-5⁴-methyl-8,11,14-trioxo-9- phenethyl-4-oxa-7,10-diaza-1(3,1)- piperidina-5(1,3)- benzenacyclotetradecaphane-12- yl)propanamide |
| 175 | 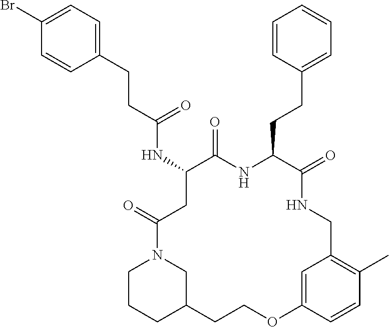 [SMILES: Cc1ccc2cc1CNC(=O)[C@H](CCc1ccccc1)NC(=O)[C@@H](NC(=O)CCc1ccc(Br)cc1)CC(=O)N1CCCC(CCO2)C1] | 3-(4-bromophenyl)-N-((9S,12S)-5⁴. methyl-8,11,14-trioxo-9-phenethyl-4-oxa- 7,10-diaza-1(3,1)-piperidina-5(1,3)- benzenacyclotetradecaphane-12- yl)propanamide |
| 176 | 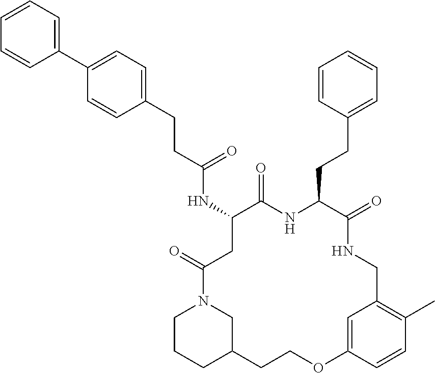 [SMILES: Cc1ccc2cc1CNC(=O)[C@H](CCc1ccccc1)NC(=O)[C@@H](NC(=O)CCc1ccc(-c3ccccc3)cc1)CC(=O)N1CCCC(CCO2)C1] | 3-([1,1′-biphenyl]-4-yl)-N-((9S,12S)-5⁴- methyl-8,11,14-trioxo-9-phenethyl-4-oxa- 7,10-diaza-1(3,1)-piperidina-5(1,3)- benzenacyclotetradecaphane-12- yl)propanamide |
| 177 | 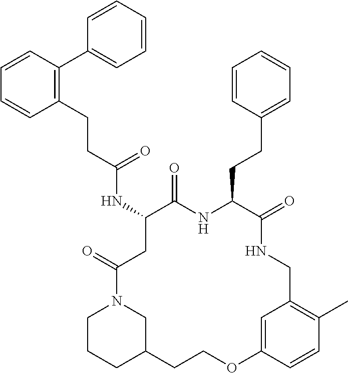 [SMILES: Cc1ccc2cc1CNC(=O)[C@H](CCc1ccccc1)NC(=O)[C@@H](NC(=O)CCc1ccccc1-c1ccccc1)CC(=O)N1CCCC(CCO2)C1] | N-((9S,12S)-5⁴-methyl-8,11,14-trioxo-9- phenethyl-4-oxa-7,10-diaza-1(3,1)- piperidina-5(1,3)- benzenacyclotetradecaphane-12-yl)-3-(2- phenoxyphenyl)propanamide |
| 178 | 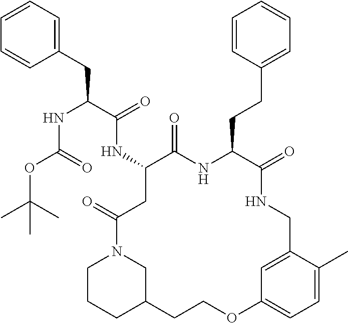 [SMILES: Cc1ccc2cc1CNC(=O)[C@H](CCc1ccccc1)NC(=O)[C@@H](NC(=O)[C@H](Cc1ccccc1)NC(=O)OC(C)(C)C)CC(=O)N1CCCC(CCO2)C1] | tert-butyl ((2S)-1-(((9S,12S)-5⁴-methyl- 8,11,14-trioxo-9-phenethyl-4-oxa-7,10- diaza-1(3,1)-piperidina-5(1,3)- benzenacyclotetradecaphane-12- yl)amino)-1-oxo-3-phenylpropan-2- yl)carbamate |
| 179 | 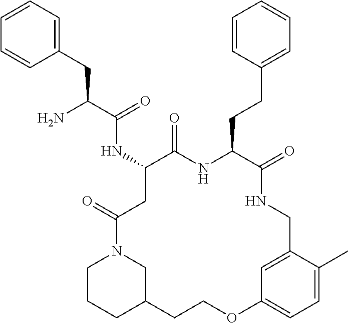 [SMILES: Cc1ccc2cc1CNC(=O)[C@H](CCc1ccccc1)NC(=O)[C@@H](NC(=O)[C@@H](N)Cc1ccccc1)CC(=O)N1CCCC(CCO2)C1] | (2R)-2-amino-N-((9S,12S)-5⁴-methyl- 8,11,14-trioxo-9-phenethyl-4-oxa-7,10- diaza-1(3,1)-piperidina-5(1,3)- benzenacyclotetradecaphane-12-yl)-3- phenylpropanamide |
| 180 | 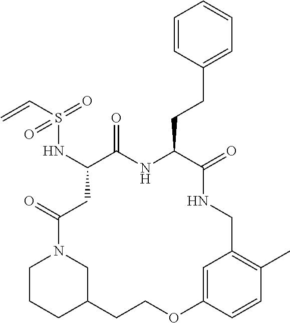 [SMILES: C=CS(=O)(=O)N[C@H]1CC(=O)N2CCCC(CCOc3ccc(C)c(c3)CNC(=O)[C@H](CCc3ccccc3)NC1=O)C2] | N-((9S,12S)-5⁴-methyl-8,11,14-trioxo-9- phenethyl-4-oxa-7,10-diaza-1(3,1)- piperidina-5(1,3)- benzenacyclotetradecaphane-12- yl)ethenesulfonamide |
| 181 | 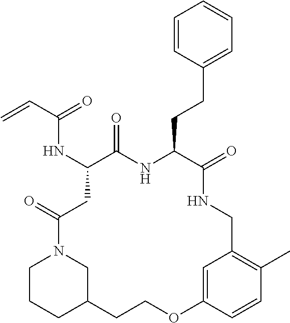 [SMILES: C=CC(=O)N[C@H]1CC(=O)N2CCCC(CCOc3ccc(C)c(c3)CNC(=O)[C@H](CCc3ccccc3)NC1=O)C2] | N-((9S,12S)-5⁴-methyl-8,11,14-trioxo-9- phenethyl-4-oxa-7,10-diaza-1(3,1)- piperidina-5(1,3)- benzenacyclotetradecaphane-12- yl)acrylamide |
| 182 | 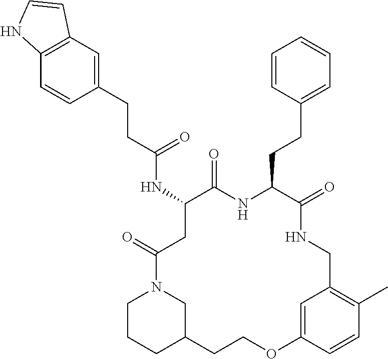 [SMILES: Cc1ccc2cc1CNC(=O)[C@H](CCc1ccccc1)NC(=O)[C@@H](NC(=O)CCc1ccc3[nH]ccc3c1)CC(=O)N1CCCC(CCO2)C1] | 3-(1H-indol-5-yl)-N-((9S,12S)-5⁴-methyl- 8,11,14-trioxo-9-phenethyl-4-oxa-7,10- diaza-1(3,1)-piperidina-5(1,3)- benzenacyclotetradecaphane-12- yl)propanamide |
| 186 | 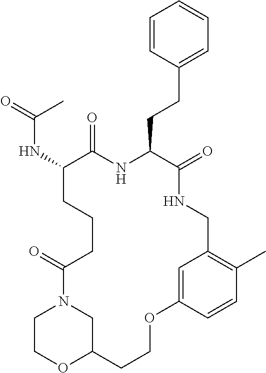 [SMILES: CC(=O)N[C@H]1CCCC(=O)N2CCOC(CCOc3ccc(C)c(c3)CNC(=O)[C@H](CCc3ccccc3)NC1=O)C2] | N-((9S,12S)-5⁴-methyl-8,11,16-trioxo-9- phenethyl-4-oxa-7,10-diaza-1(2,4)- morpholina-5(1,3)- benzenacyclohexadecaphane-12- yl)acetamide |
| 191 | 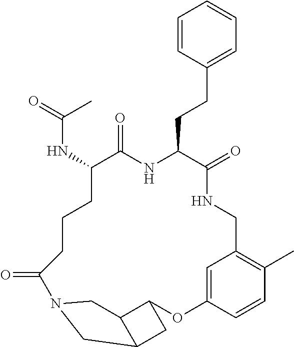 [SMILES: CC(=O)N[C@H]1CCCC(=O)N2CC3CC(Oc4ccc(C)c(c4)CNC(=O)[C@H](CCc4ccccc4)NC1=O)C3C2] | N-((11R,15S,7S,10S)-3⁴-methyl-6,9,14- trioxo-7-phenethyl-2-oxa-13,5,8-triaza- 1(7,3)-bicyclo[3.2.0]heptana-3(1,3)- benzenacyclotetradecaphane-10- yl)acetamide |
| 195 | 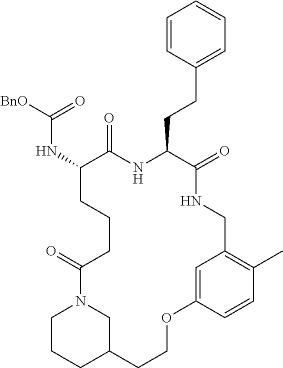 [SMILES: Cc1ccc2cc1CNC(=O)[C@H](CCc1ccccc1)NC(=O)[C@@H](NC(=O)OCc1ccccc1)CCCC(=O)N1CCCC(CCO2)C1] | benzyl ((9S,12S)-5⁴-methyl-8,11,16- trioxo-9-phenethyl-4-oxa-7,10-diaza- 1(3,1)-piperidina-5(1,3)- benzenacyclohexadecaphane-12- yl)carbamate |
| 199 | 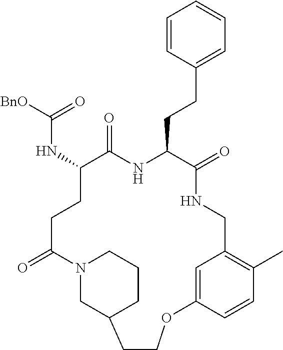 [SMILES: Cc1ccc2cc1CNC(=O)[C@H](CCc1ccccc1)NC(=O)[C@@H](NC(=O)OCc1ccccc1)CCC(=O)N1CCCC(CCO2)C1] | benzyl ((9S,12S)-5⁴-methyl-8,11,15- trioxo-9-phenethyl-4-oxa-7,10-diaza- 1(3,1)-piperidina-5(1,3)- benzenacyclopentadecaphane-12- yl)carbamate |
| 205 | 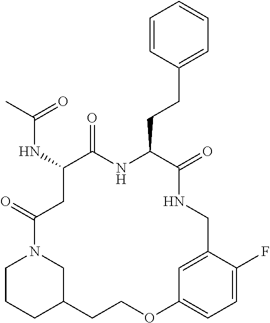 [SMILES: CC(=O)N[C@H]1CC(=O)N2CCCC(CCOc3ccc(F)c(c3)CNC(=O)[C@H](CCc3ccccc3)NC1=O)C2] | N-((9S,12S)-5⁴-fluoro-8,11,14-trioxo-9- phenethyl-4-oxa-7,10-diaza-1(3,1)- piperidina-5(1,3)- benzenacyclotetradecaphane-12- yl)acetamide |
| 211 | 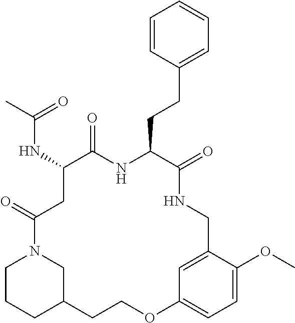 [SMILES: COc1ccc2cc1CNC(=O)[C@H](CCc1ccccc1)NC(=O)[C@@H](NC(C)=O)CC(=O)N1CCCC(CCO2)C1] | N-((9S,12S)-5⁴-methoxy-8,11,14-trioxo-9- phenethyl-4-oxa-7,10-diaza-1(3,1)- piperidina-5(1,3)- benzenacyclotetradecaphane-12- yl)acetamide |
| 212 | 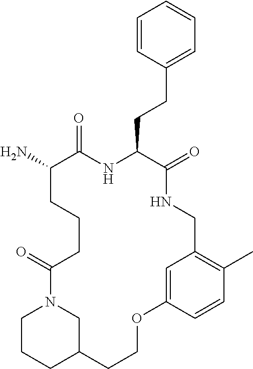 [SMILES: Cc1ccc2cc1CNC(=O)[C@H](CCc1ccccc1)NC(=O)[C@@H](N)CCCC(=O)N1CCCC(CCO2)C1] | (9S,12S)-5⁴-methyl-12-amino-8,11,16- trioxo-9-phenethyl-4-oxa-7,10-diaza- 1(3,1)-piperidina-5(1,3)- benzenacyclohexadecaphane |
| 213 | 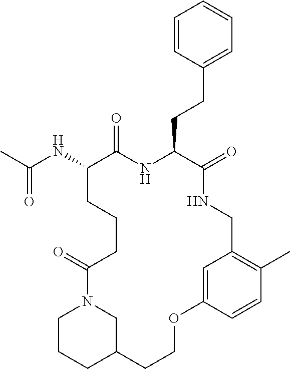 [SMILES: CC(=O)N[C@H]1CCCC(=O)N2CCCC(CCOc3ccc(C)c(c3)CNC(=O)[C@H](CCc3ccccc3)NC1=O)C2] | N-((9S,12S)-5⁴-methyl-8,11,16-trioxo-9- phenethyl-4-oxa-7,10-diaza-1(3,1)- piperidina-5(1,3)- benzenacyclohexadecaphane-12- yl)acetamide |
| 214 | 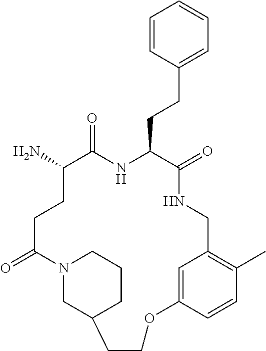 [SMILES: Cc1ccc2cc1CNC(=O)[C@H](CCc1ccccc1)NC(=O)[C@@H](N)CCC(=O)N1CCCC(CCO2)C1] | (9S,12S)-12-amino-5⁴-methyl-8,11,15- trioxo-9-phenethyl-4-oxa-7,10-diaza- 1(3,1)-piperidina-5(1,3)- benzenacyclopentadecaphane) |
| 215 | 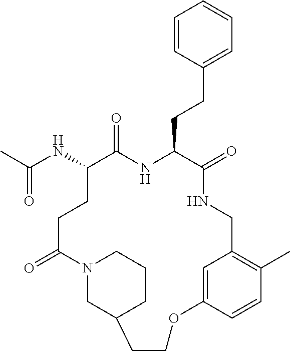 [SMILES: CC(=O)N[C@H]1CCC(=O)N2CCCC(CCOc3ccc(C)c(c3)CNC(=O)[C@H](CCc3ccccc3)NC1=O)C2] | N-((9S,12S)-5⁴-methyl-8,11,15-trioxo-9- phenethyl-4-oxa-7,10-diaza-1(3,1)- piperidina-5(1,3)- benzenacyclopentadecaphane-12- yl)acetamide |
| 216 | 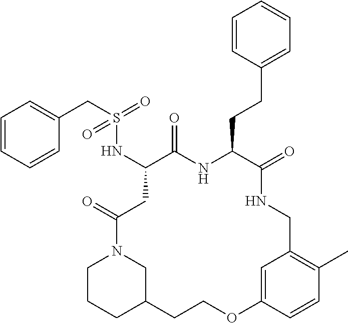 [SMILES: Cc1ccc2cc1CNC(=O)[C@H](CCc1ccccc1)NC(=O)[C@@H](NS(=O)(=O)Cc1ccccc1)CC(=O)N1CCCC(CCO2)C1] | N-((9S,12S)-5⁴-methyl-8,11,14-trioxo-9- phenethyl-4-oxa-7,10-diaza-1(3,1)- piperidina-5(1,3)- benzenacyclotetradecaphane-12-yl)-1- phenylmethanesulfonamide |
| 217 | 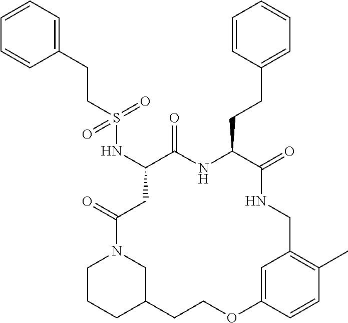 [SMILES: Cc1ccc2cc1CNC(=O)[C@H](CCc1ccccc1)NC(=O)[C@@H](NS(=O)(=O)CCc1ccccc1)CC(=O)N1CCCC(CCO2)C1] | N-((9S,12S)-5⁴-methyl-8,11,14-trioxo-9- phenethyl-4-oxa-7,10-diaza-1(3,1)- piperidina-5(1,3)- benzenacyclotetradecaphane-12-yl)-2- phenylethane-1-sulfonamide |
| 218 | 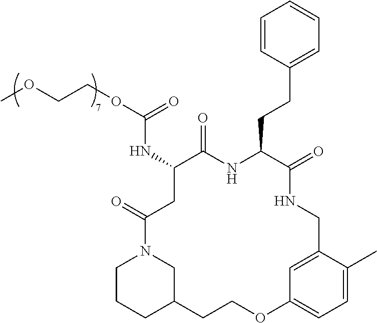 | 2,5,8,11,14,17,20-heptaoxadocosan-22-yl ((9R,12R)-5⁴-methyl-8,11,14-trioxo-9- phenethyl-4-oxa-7,10-diaza-1(3,1)- piperidina-5(1,3)- benzenacyclotetradecaphane-12- yl)carbamate |
| 219 | 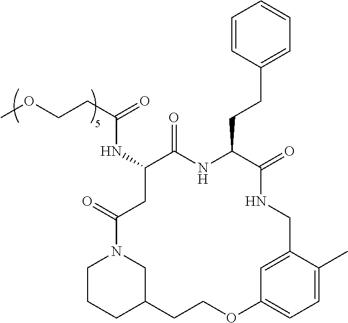 | N-((9S,12S)-5⁴-methyl-8,11,14-trioxo-9- phenethyl-4-oxa-7,10-diaza-1(3,1)- piperidina-5(1,3)- benzenacyclotetradecaphane-12-yl)- 2,5,8,11,14-pentaoxaheptadecan-17- amide |
| 220 | 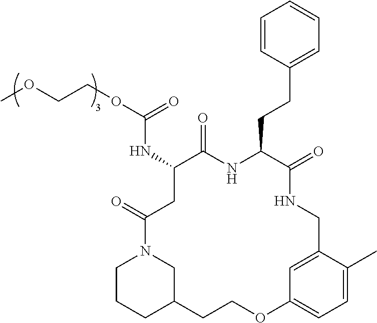 | ((9R,12R)-5⁴-methyl-8,11,14-trioxo-9- phenethyl-4-oxa-7,10-diaza-1(3,1)- piperidina-5(1,3)- benzenacyclotetradecaphane-12- yl)carbamate |
| 252 | 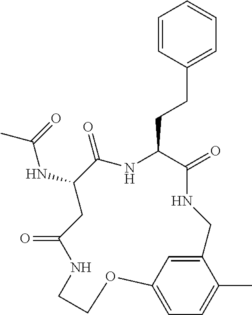 [SMILES: CC(=O)N[C@H]1CC(=O)NCCOc2ccc(C)c(c2)CNC(=O)[C@H](CCc2ccccc2)NC1=O] | N-((8S,11S)-1⁴-methyl-6,9,12-trioxo-11- phenethyl-2-oxa-5,10,13-triaza-1(1,3)- benzenacyclotetradecaphane-8- yl)acetamide |
| 253 | 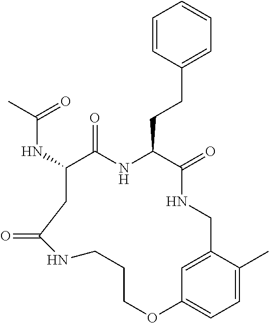 [SMILES: CC(=O)N[C@H]1CC(=O)NCCCOc2ccc(C)c(c2)CNC(=O)[C@H](CCc2ccccc2)NC1=O] | N-((9S,12S)-1⁴-methyl-7,10,13-trioxo-12- phenethyl-2-oxa-6,11,14-triaza-1(1,3)- benzenacyclopentadecaphane-9- yl)acetamide |
| 254 | 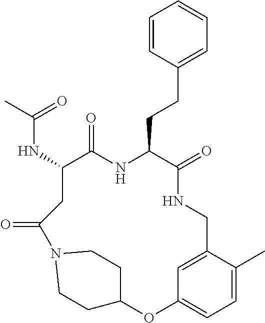 [SMILES: CC(=O)N[C@H]1CC(=O)N2CCC(CC2)Oc2ccc(C)c(c2)CNC(=O)[C@H](CCc2ccccc2)NC1=O] | N-((7S,10S)-3⁴-methyl-6,9,12-trioxo-7- phenethyl-2-oxa-5,8-diaza-1(4,1)- piperidina-3(1,3)- benzenacyclododecaphane-10- yl)acetamide |
| 255 | 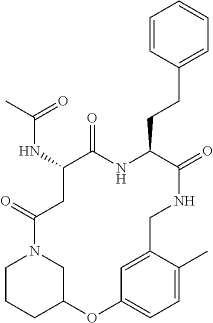 [SMILES: CC(=O)N[C@H]1CC(=O)N2CCCC(C2)Oc2ccc(C)c(c2)CNC(=O)[C@H](CCc2ccccc2)NC1=O] | N-((7S,10S)-3⁴-methyl-6,9,12-trioxo-7- phenethyl-2-oxa-5,8-diaza-1(3,1)- piperidina-3(1,3)- benzenacyclododecaphane-10- yl)acetamide |
| 256 | 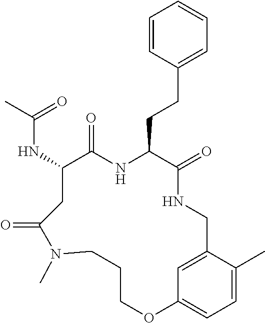 [SMILES: CC(=O)N[C@H]1CC(=O)N(C)CCCOc2ccc(C)c(c2)CNC(=O)[C@H](CCc2ccccc2)NC1=O] | N-((9S,12S)-1⁴,6-dimethyl-7,10,13-trioxo- 12-phenethyl-2-oxa-6,11,14-triaza-1(1,3)- benzenacyclopentadecaphane-9- yl)acetamide |
| 257 | 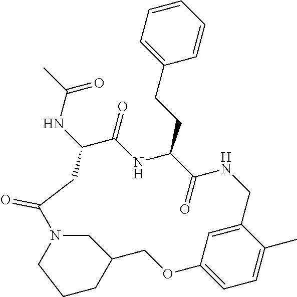 [SMILES: CC(=O)N[C@H]1CC(=O)N2CCCC(COc3ccc(C)c(c3)CNC(=O)[C@H](CCc3ccccc3)NC1=O)C2] | N-((8S,11S)-4⁴-methyl-7,10,13-trioxo-8- phenethyl-3-oxa-6,9-diaza-1(3,1)- piperidina-4(1,3)- benzenacyclotridecaphane-11- yl)acetamide |
| 258 | 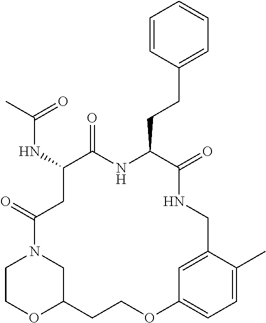 [SMILES: CC(=O)N[C@H]1CC(=O)N2CCOC(CCOc3ccc(C)c(c3)CNC(=O)[C@H](CCc3ccccc3)NC1=O)C2] | N-((9S,12S)-5⁴-methyl-8,11,14-trioxo-9- phenethyl-4-oxa-7,10-diaza-1(2,4)- morpholina-5(1,3)- benzenacyclotetradecaphane-12- yl)acetamide |
| 259 | 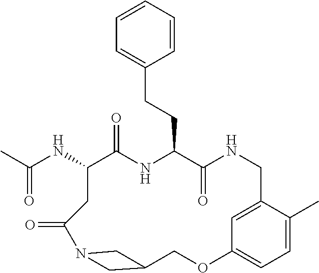 [SMILES: CC(=O)N[C@H]1CC(=O)N2CC(COc3ccc(C)c(c3)CNC(=O)[C@H](CCc3ccccc3)NC1=O)C2] | N-((8S,11S)-4⁴-methyl-7,10,13-trioxo-8- phenethyl-3-oxa-6,9-diaza-1(3,1)- azetidina-4(1,3)- benzenacyclotridecaphane-11- yl)acetamide |
| 260 | 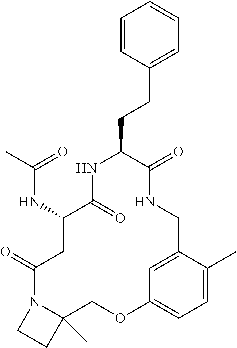 [SMILES: CC(=O)N[C@H]1CC(=O)N2CCC2(C)COc2ccc(C)c(c2)CNC(=O)[C@H](CCc2ccccc2)NC1=O] | N-((8S,11S)-1²,4⁴-dimethyl-7,10,13- trioxo-8-phenethyl-3-oxa-6,9-diaza-1(2,1)- azetidina-4(1,3)- benzenacyclotridecaphane-11- yl)acetamide |
| 261 | 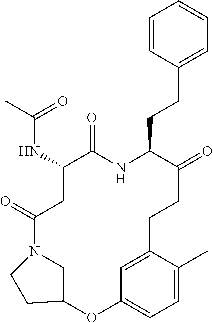 [SMILES: CC(=O)N[C@H]1CC(=O)N2CCC(C2)Oc2ccc(C)c(c2)CCC(=O)[C@H](CCc2ccccc2)NC1=O] | N-((7S,10S)-3⁴-methyl-6,9,12-trioxo-7- phenethyl-2-oxa-5,8-diaza-1(3,1)- pyrrolidina-3(1,3)- benzenacyclododecaphane-10- yl)acetamide |
| 265 | 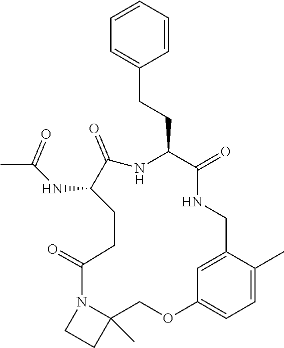 [SMILES: CC(=O)N[C@H]1CCC(=O)N2CCC2(C)COc2ccc(C)c(c2)CNC(=O)[C@H](CCc2ccccc2)NC1=O] | N-((8S,11S)-1²,4⁴-dimethyl-7,10,14- trioxo-8-phenethyl-3-oxa-6,9-diaza-1(2,1)- azetidina-4(1,3)- benzenacyclotetradecaphane-11- yl)acetamide |
| 269 | 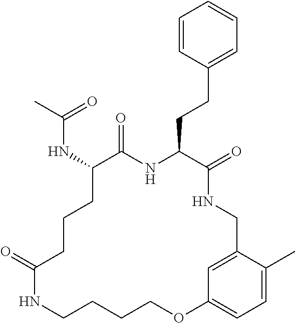 [SMILES: CC(=O)N[C@H]1CCCC(=O)NCCCCOc2ccc(C)c(c2)CNC(=O)[C@H](CCc2ccccc2)NC1=O] | N-((12S,15S)-1⁴-methyl-8,13,16-trioxo- 15-phenethyl-2-oxa-7,14,17-triaza-1(1,3)- benzenacyclooctadecaphane-12- yl)acetamide |
| 280 | 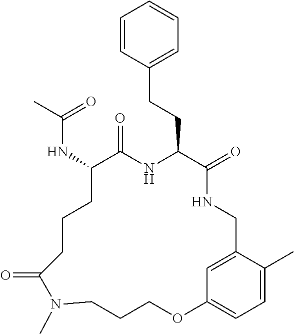 [SMILES: CC(=O)N[C@H]1CCCC(=O)N(C)CCCOc2ccc(C)c(c2)CNC(=O)[C@H](CCc2ccccc2)NC1=O] | N-((11S,14S)-1⁴,6-dimethyl-7,12,15- trioxo-1⁴-phenethyl-2-oxa-6,13,16-triaza- 1(1,3)-benzenacycloheptadecaphane-11- yl)acetamide |
| 281 | 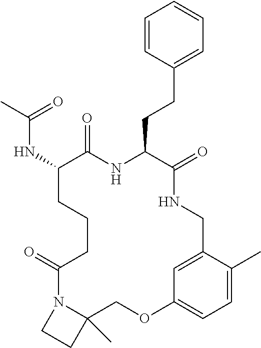 [SMILES: CC(=O)N[C@H]1CCCC(=O)N2CCC2(C)COc2ccc(C)c(c2)CNC(=O)[C@H](CCc2ccccc2)NC1=O] | N-((8S,11S)-1²,4⁴-dimethyl-7,10,15- trioxo-8-phenethyl-3-oxa-6,9-diaza-1(2,1)- azetidina-4(1,3)- benzenacyclopentadecaphane-11- yl)acetamide |
| 282 | 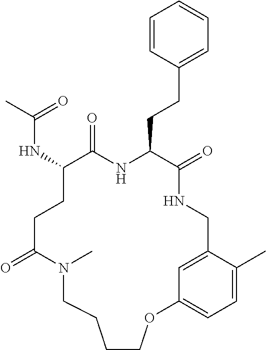 [SMILES: CC(=O)N[C@H]1CCC(=O)N(C)CCCCOc2ccc(C)c(c2)CNC(=O)[C@H](CCc2ccccc2)NC1=O] | N-((11S,14S)-1⁴,7-dimethyl-8,12,15- trioxo-1⁴-phenethyl-2-oxa-7,13,16-triaza- 1(1,3)-benzenacycloheptadecaphane-11- yl)acetamide |
| 283 | 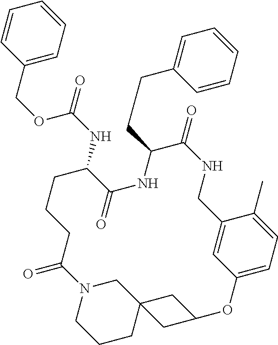 [SMILES: Cc1ccc2cc1CNC(=O)[C@H](CCc1ccccc1)NC(=O)[C@@H](NC(=O)OCc1ccccc1)CCCC(=O)N1CCCC3(CC(C3)O2)C1] | benzyl ((7S,10S)-3⁴-methyl-6,9,14-trioxo- 7-phenethyl-2-oxa-5,8-diaza-15(3,1)- piperidina-3(1,3)-benzena-1(1,3)- cyclobutanapentadecaphane-10- yl)carbamate |
| 284 | 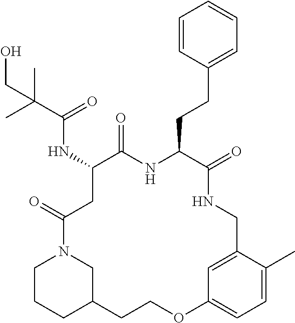 [SMILES: Cc1ccc2cc1CNC(=O)[C@H](CCc1ccccc1)NC(=O)[C@@H](NC(=O)C(C)(C)CO)CC(=O)N1CCCC(CCO2)C1] | 3-hydroxy-2,2-dimethyl-N-((9S,12S)-5⁴ methyl-8,11,14-trioxo-9-phenethyl-4-oxa- 7,10-diaza-1(3,1)-piperidina-5(1,3)- benzenacyclotetradecaphane-12- yl)propanamide |
| 285 | 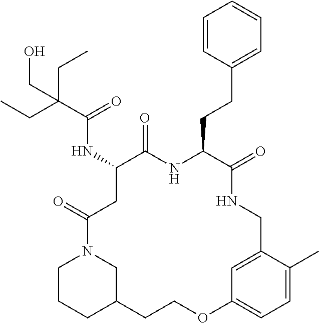 [SMILES: CCC(CC)(CO)C(=O)N[C@H]1CC(=O)N2CCCC(CCOc3ccc(C)c(c3)CNC(=O)[C@H](CCc3ccccc3)NC1=O)C2] | 2-ethyl-2-(hydroxymethyl)-N-((9S,12S)- 5⁴-methyl-8,11,14-trioxo-9-phenethyl-4- oxa-7,10-diaza-1(3,1)-piperidina-5(1,3)- benzenacyclotetradecaphane-12- yl)butanamide |
| 286 | 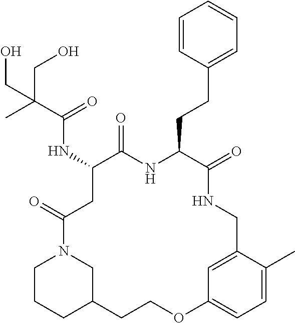 [SMILES: Cc1ccc2cc1CNC(=O)[C@H](CCc1ccccc1)NC(=O)[C@@H](NC(=O)C(C)(CO)CO)CC(=O)N1CCCC(CCO2)C1] | 3-hydroxy-2-(hydroxymethyl)-2-methyl-N- ((9S,12S)-5⁴-methyl-8,11,14-trioxo-9- phenethyl-4-oxa-7,10-diaza-1(3,1)- piperidina-5(1,3)- benzenacyclotetradecaphane-12- yl)propanamide |
| 287 | 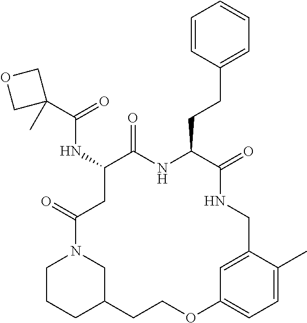 [SMILES: Cc1ccc2cc1CNC(=O)[C@H](CCc1ccccc1)NC(=O)[C@@H](NC(=O)C1(C)COC1)CC(=O)N1CCCC(CCO2)C1] | 3-methyl-N-((9S,12S)-5⁴-methyl-8,11,14- trioxo-9-phenethyl-4-oxa-7,10-diaza- 1(3,1)-piperidina-5(1,3)- benzenacyclotetradecaphane-12- yl)oxetane-3-carboxamide |
| 288 | 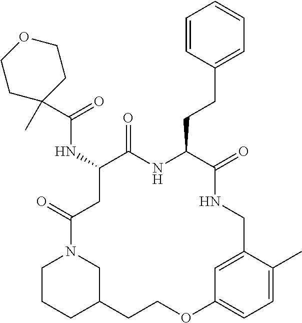 [SMILES: Cc1ccc2cc1CNC(=O)[C@H](CCc1ccccc1)NC(=O)[C@@H](NC(=O)C1(C)CCOCC1)CC(=O)N1CCCC(CCO2)C1] | 4-methyl-N-((9S,12S)-5⁴-methyl-8,11,14- trioxo-9-phenethyl-4-oxa-7,10-diaza- 1(3,1)-piperidina-5(1,3)- benzenacyclotetradecaphane-12- yl)tetrahydro-2H-pyran-4-carboxamide |
| 289 | 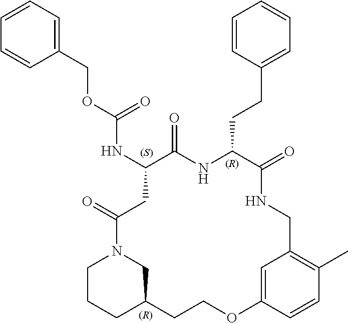 | benzyl ((13R,9R,12S)-5⁴-methyl-8,11,14- trioxo-9-phenethyl-4-oxa-7,10-diaza- 1(3,1)-piperidina-5(1,3)- benzenacyclotetradecaphane-12- yl)carbamate |
| 290 | 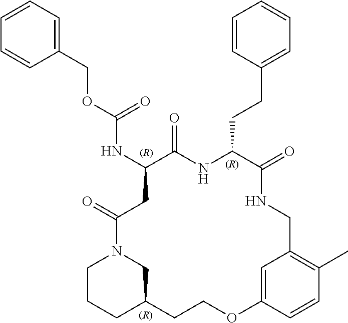 | benzyl ((13R,9R,12R)-5⁴-methyl-8,11,14- trioxo-9-phenethyl-4-oxa-7,10-diaza- 1(3,1)-piperidina-5(1,3)- benzenacyclotetradecaphane-12- yl)carbamate |
| 291 | 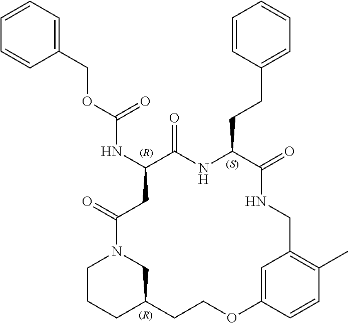 | benzyl ((13R,9S,12R)-5⁴-methyl-8,11,14- trioxo-9-phenethyl-4-oxa-7,10-diaza- 1(3,1)-piperidina-5(1,3)- benzenacyclotetradecaphane-12- yl)carbamate |
| 292 | 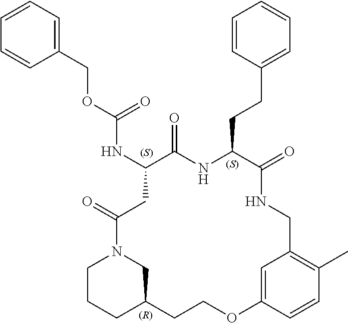 | benzyl ((13R,9S,12S)-5⁴-methyl-8,11,14- trioxo-9-phenethyl-4-oxa-7,10-diaza- 1(3,1)-piperidina-5(1,3)- benzenacyclotetradecaphane-12- yl)carbamate |
| 293 | 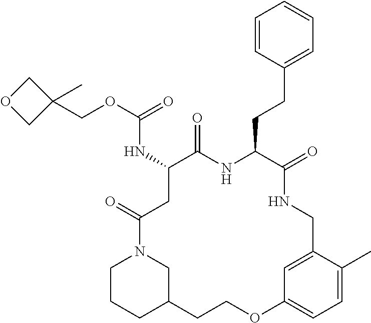 [SMILES: Cc1ccc2cc1CNC(=O)[C@H](CCc1ccccc1)NC(=O)[C@@H](NC(=O)OCC1(C)COC1)CC(=O)N1CCCC(CCO2)C1] | (3-methyloxetan-3-yl)methyl ((9S,12S)-5⁴- methyl-8,11,14-trioxo-9-phenethyl-4-oxa- 7,10-diaza-1(3,1)-piperidina-5(1,3)- benzenacyclotetradecaphane-12- yl)carbamate |
| 294 | 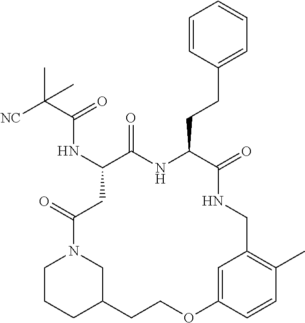 [SMILES: Cc1ccc2cc1CNC(=O)[C@H](CCc1ccccc1)NC(=O)[C@@H](NC(=O)C(C)(C)C#N)CC(=O)N1CCCC(CCO2)C1] | (3-methyloxetan-3-yl)methyl ((9S,12S)-5⁴- methyl-8,11,14-trioxo-9-phenethyl-4-oxa- 7,10-diaza-1(3,1)-piperidina-5(1,3)- benzenacyclotetradecaphane-12- yl)carbamate |
| 295 | 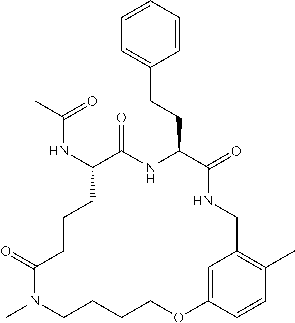 | N-(1⁴,7-dimethyl-8,13,16-trioxo-15- phenethyl-2-oxa-7,14,17-triaza-1(1,3)- benzenacyclooctadecaphane-12- yl)acetamide |
| 296 | 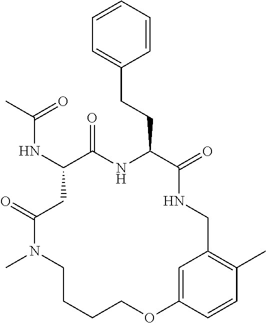 [SMILES: CC(=O)N[C@H]1CC(=O)N(C)CCCCOc2ccc(C)c(c2)CNC(=O)[C@H](CCc2ccccc2)NC1=O] | N-(1⁴,7-dimethyl-8,11,14-trioxo-13- phenethyl-2-oxa-7,12,15-triaza-1(1,3)- benzenacyclohexadecaphane-10- yl)acetamide |
| 298 | 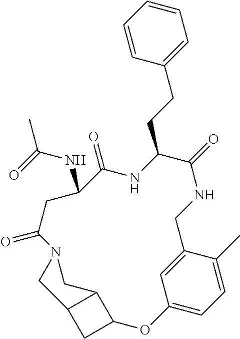 [SMILES: CC(=O)N[C@H]1CC(=O)N2CC3CC(Oc4ccc(C)c(c4)CNC(=O)[C@H](CCc4ccccc4)NC1=O)C3C2] | N-(3⁴-methyl-6,9,12-trioxo-7-phenethyl-2- oxa-13,5,8-triaza-1(6,3)- bicyclo[3.2.0]heptana-3(1,3)- benzenacyclododecaphane-10- yl)acetamide |
| 299 | 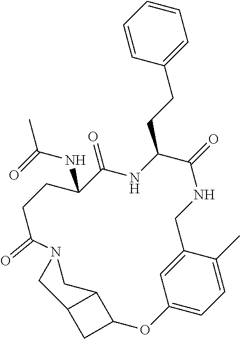 [SMILES: CC(=O)N[C@H]1CCC(=O)N2CC3CC(Oc4ccc(C)c(c4)CNC(=O)[C@H](CCc4ccccc4)NC1=O)C3C2] | N-(3⁴-methyl-6,9,13-trioxo-7-phenethyl-2- oxa-13,5,8-triaza-1(6,3)- bicyclo[3.2.0]heptana-3(1,3)- benzenacyclotridecaphane-10- yl)acetamide |
| 300 | 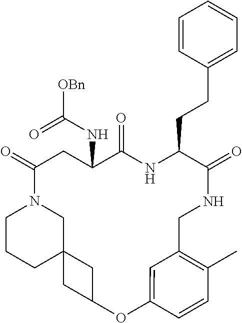 [SMILES: Cc1ccc2cc1CNC(=O)[C@H](CCc1ccccc1)NC(=O)[C@@H](NC(=O)OCc1ccccc1)CC(=O)N1CCCC3(CC(C3)O2)C1] | benzyl ((7S,10S)-3⁴-methyl-6,9,12-trioxo- 7-phenethyl-2-oxa-5,8-diaza-13(3,1)- piperidina-3(1,3)-benzena-1(1,3)- cyclobutanatridecaphane-10- yl)carbamate |
| 301 | 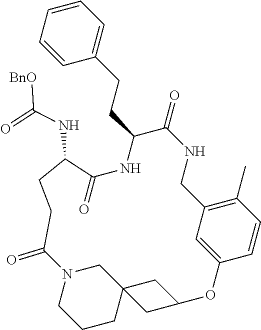 [SMILES: Cc1ccc2cc1CNC(=O)[C@H](CCc1ccccc1)NC(=O)[C@@H](NC(=O)OCc1ccccc1)CCC(=O)N1CCCC3(CC(C3)O2)C1] | benzyl ((7S,10S)-3⁴-methyl-6,9,13-trioxo- 7-phenethyl-2-oxa-5,8-diaza-14(3,1)- piperidina-3(1,3)-benzena-1(1,3)- cyclobutanatetradecaphane-10- yl)carbamate |
| 302 | 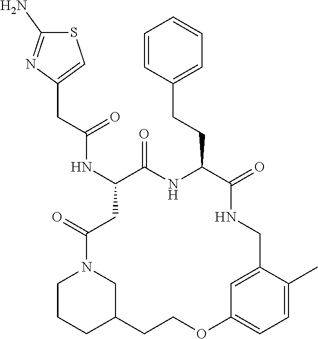 [SMILES: Cc1ccc2cc1CNC(=O)[C@H](CCc1ccccc1)NC(=O)[C@@H](NC(=O)Cc1csc(N)n1)CC(=O)N1CCCC(CCO2)C1] | 2-(2-aminothiazol-4-yl)-N-((9S,12S)-5⁴- methyl-8,11,14-trioxo-9-phenethyl-4-oxa- 7,10-diaza-1(3,1)-piperidina-5(1,3)- benzenacyclotetradecaphane-12- yl)acetamide |
| 303 | 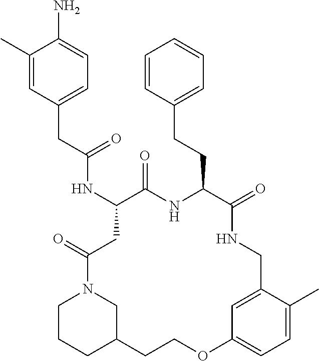 | 2-(4-amino-3-methylphenyl)-N-((9S,12S)- 5⁴-methyl-8,11,14-trioxo-9-phenethyl-4- oxa-7,10-diaza-1(3,1)-piperidina-5(1,3)- benzenacyclotetradecaphane-12- yl)acetamide |
| 304 | 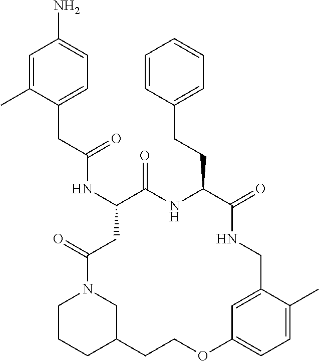 | 2-(4-amino-2-methylphenyl)-N-((9S,12S)- 5⁴-methyl-8,11,14-trioxo-9-phenethyl-4- oxa-7,10-diaza-1(3,1)-piperidina-5(1,3)- benzenacyclotetradecaphane-12- yl)acetamide |
| 305 | 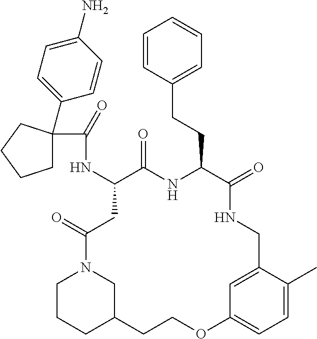 [SMILES: Cc1ccc2cc1CNC(=O)[C@H](CCc1ccccc1)NC(=O)[C@@H](NC(=O)C1(c3ccc(N)cc3)CCCC1)CC(=O)N1CCCC(CCO2)C1] | 1-(4-aminophenyl)-N-((9S,12S)-5⁴- methyl-8,11,14-trioxo-9-phenethyl-4-oxa- 7,10-diaza-1(3,1)-piperidina-5(1,3)- benzenacyclotetradecaphane-12- yl)cyclopentane-1-carboxamide |
| 306 | 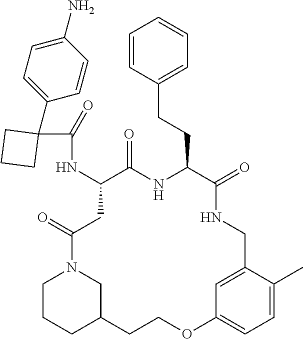 [SMILES: Cc1ccc2cc1CNC(=O)[C@H](CCc1ccccc1)NC(=O)[C@@H](NC(=O)C1(c3ccc(N)cc3)CCC1)CC(=O)N1CCCC(CCO2)C1] | 1-(4-aminophenyl)-N-((9S,12S)-5⁴- methyl-8,11,14-trioxo-9-phenethyl-4-oxa- 7,10-diaza-1(3,1)-piperidina-5(1,3)- benzenacyclotetradecaphane-12- yl)cyclobutane-1-carboxamide |
| 307 | 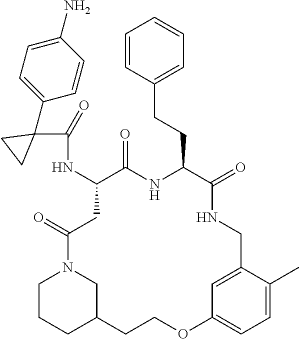 [SMILES: Cc1ccc2cc1CNC(=O)[C@H](CCc1ccccc1)NC(=O)[C@@H](NC(=O)C1(c3ccc(N)cc3)CC1)CC(=O)N1CCCC(CCO2)C1] | 1-(4-aminophenyl)-N-((9S,12S)-5⁴- methyl-8,11,14-trioxo-9-phenethyl-4-oxa- 7,10-diaza-1(3,1)-piperidina-5(1,3)- benzenacyclotetradecaphane-12- yl)cyclopropane-1-carboxamide |
| 308 | 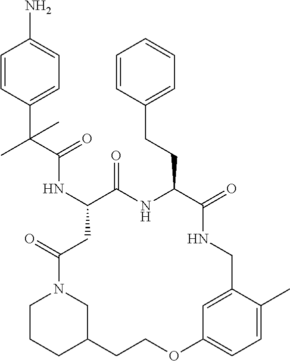 [SMILES: Cc1ccc2cc1CNC(=O)[C@H](CCc1ccccc1)NC(=O)[C@@H](NC(=O)C(C)(C)c1ccc(N)cc1)CC(=O)N1CCCC(CCO2)C1] | 2-(4-aminophenyl)-2-methyl-N-((9S,12S)- 5⁴-methyl-8,11,14-trioxo-9-phenethyl-4- oxa-7,10-diaza-1(3,1)-piperidina-5(1,3)- benzenacyclotetradecaphane-12- yl)propanamide |
| 309 | 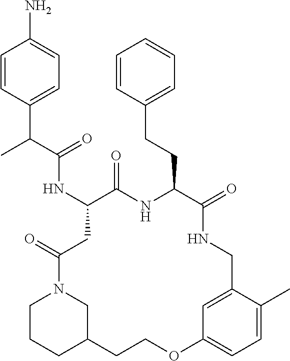 [SMILES: Cc1ccc2cc1CNC(=O)[C@H](CCc1ccccc1)NC(=O)[C@@H](NC(=O)C(C)c1ccc(N)cc1)CC(=O)N1CCCC(CCO2)C1] | 2-(4-aminophenyl)-N-((9S,12S)-5⁴- methyl-8,11,14-trioxo-9-phenethyl-4-oxa- 7,10-diaza-1(3,1)-piperidina-5(1,3)- benzenacyclotetradecaphane-12- yl)propanamide |
| 310 | 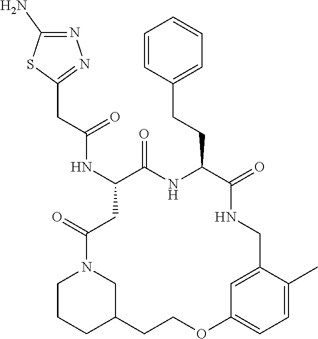 [SMILES: Cc1ccc2cc1CNC(=O)[C@H](CCc1ccccc1)NC(=O)[C@@H](NC(=O)Cc1nnc(N)s1)CC(=O)N1CCCC(CCO2)C1] | 2-(5-amino-1,3,4-thiadiazol-2-yl)-N- ((9S,12S)-5⁴-methyl-8,11,14-trioxo-9- phenethyl-4-oxa-7,10-diaza-1(3,1)- piperidina-5(1,3)- benzenacyclotetradecaphane-12- yl)acetamide |
| 311 | 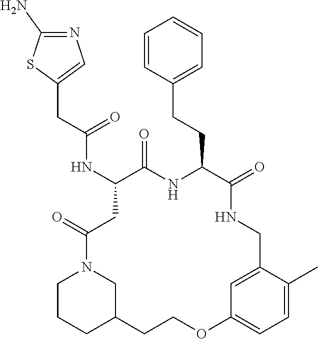 [SMILES: Cc1ccc2cc1CNC(=O)[C@H](CCc1ccccc1)NC(=O)[C@@H](NC(=O)Cc1cnc(N)s1)CC(=O)N1CCCC(CCO2)C1] | 2-(2-aminothiazol-5-yl)-N-((9S,12S)-5⁴- methyl-8,11,14-trioxo-9-phenethyl-4-oxa- 7,10-diaza-1(3,1)-piperidina-5(1,3)- benzenacyclotetradecaphane-12- yl)acetamide |
| 312 | 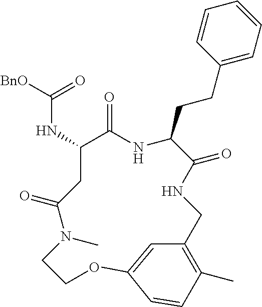 [SMILES: Cc1ccc2cc1CNC(=O)[C@H](CCc1ccccc1)NC(=O)[C@@H](NC(=O)OCc1ccccc1)CC(=O)N(C)CCO2] | benzyl ((8S,11S)-1⁴,5-dimethyl-6,9,12- trioxo-11-phenethyl-2-oxa-5,10,13-triaza- 1(1,3)-benzenacyclotetradecaphane-8- yl)carbamate |
| 313 | 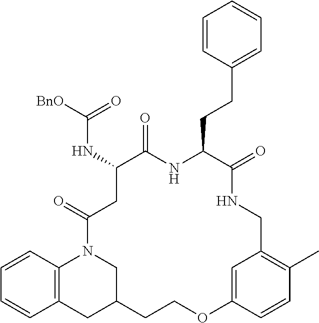 [SMILES: Cc1ccc2cc1CNC(=O)[C@H](CCc1ccccc1)NC(=O)[C@@H](NC(=O)OCc1ccccc1)CC(=O)N1CC(CCO2)Cc2ccccc21] | benzyl ((9S,12S)-5⁴-methyl-8,11,14- trioxo-9-phenethyl-11,12,13,14- tetrahydro-4-oxa-7,10-diaza-1(3,1)- quinolina-5(1,3)- benzenacyclotetradecaphane-12- yl)carbamate |
| 314 | 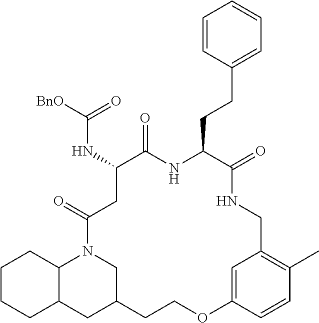 [SMILES: Cc1ccc2cc1CNC(=O)[C@H](CCc1ccccc1)NC(=O)[C@@H](NC(=O)OCc1ccccc1)CC(=O)N1CC(CCO2)CC2CCCCC21] | benzyl ((9S,12S)-5⁴-methyl-8,11,14- trioxo-9-phenethyl- 11,12,13,14,14a,15,16,17,18,18a- decahydro-4-oxa-7,10-diaza-1(3,1)- quinolina-5(1,3)- benzenacyclotetradecaphane-12- yl)carbamate |
| 315 | 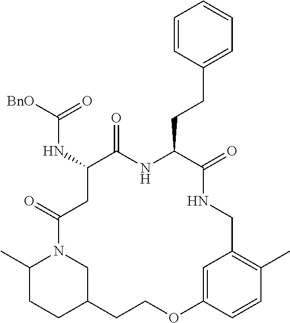 | benzyl ((9S,12S)-16,5⁴-dimethyl-8,11,14- trioxo-9-phenethyl-4-oxa-7,10-diaza- 1(3,1)-piperidina-5(1,3)- benzenacyclotetradecaphane-12- yl)carbamate |
| 316 | 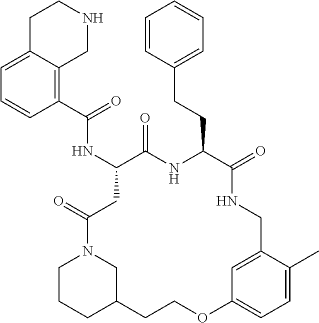 [SMILES: Cc1ccc2cc1CNC(=O)[C@H](CCc1ccccc1)NC(=O)[C@@H](NC(=O)c1cccc3c1CNCC3)CC(=O)N1CCCC(CCO2)C1] | N-((9S,12S)-5⁴-methyl-8,11,14-trioxo-9- phenethyl-4-oxa-7,10-diaza-1(3,1)- piperidina-5(1,3)- benzenacyclotetradecaphane-12-yl)- 1,2,3,4-tetrahydroisoquinoline-5- carboxamide |
| 317 | 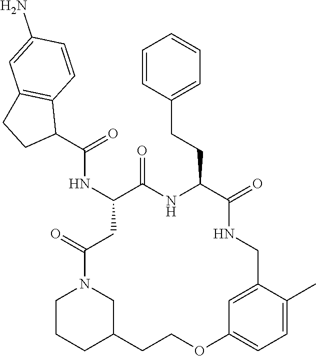 [SMILES: Cc1ccc2cc1CNC(=O)[C@H](CCc1ccccc1)NC(=O)[C@@H](NC(=O)C1CCc3cc(N)ccc31)CC(=O)N1CCCC(CCO2)C1] | 5-amino-N-((9S,12S)-5⁴-methyl-8,11,14- trioxo-9-phenethyl-4-oxa-7,10-diaza- 1(3,1)-piperidina-5(1,3)- benzenacyclotetradecaphane-12-yl)-2,3- dihydro-1H-indene-1-carboxamide |
| 318 | 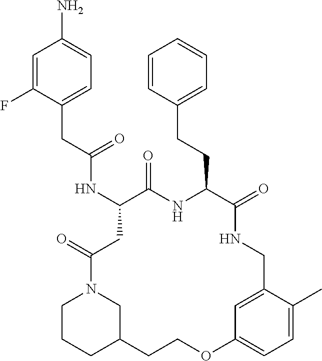 [SMILES: Cc1ccc2cc1CNC(=O)[C@H](CCc1ccccc1)NC(=O)[C@@H](NC(=O)Cc1ccc(N)cc1F)CC(=O)N1CCCC(CCO2)C1] | 2-(4-amino-2-fluorophenyl)-N-((9S,12S)- 5⁴-methyl-8,11,14-trioxo-9-phenethyl-4- oxa-7,10-diaza-1(3,1)-piperidina-5(1,3)- benzenacyclotetradecaphane-12- yl)acetamide |
| 319 | 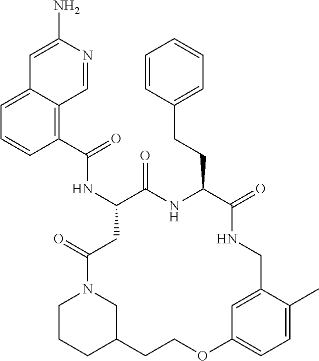 [SMILES: Cc1ccc2cc1CNC(=O)[C@H](CCc1ccccc1)NC(=O)[C@@H](NC(=O)c1cccc3cc(N)ncc13)CC(=O)N1CCCC(CCO2)C1] | 3-amino-N-((9S,12S)-5⁴-methyl-8,11,14- trioxo-9-phenethyl-4-oxa-7,10-diaza- 1(3,1)-piperidina-5(1,3)- benzenacyclotetradecaphane-12- yl)isoquinoline-8-carboxamide |
| 320 | 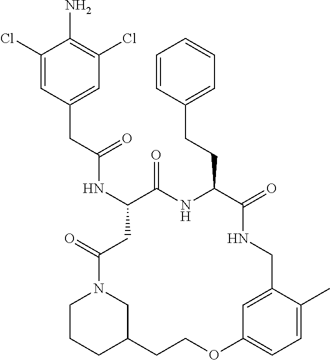 [SMILES: Cc1ccc2cc1CNC(=O)[C@H](CCc1ccccc1)NC(=O)[C@@H](NC(=O)Cc1cc(Cl)c(N)c(Cl)c1)CC(=O)N1CCCC(CCO2)C1] | 2-(4-amino-3,5-dichlorophenyl)-N- ((9S,12S)-5⁴-methyl-8,11,14-trioxo-9- phenethyl-4-oxa-7,10-diaza-1(3,1)- piperidina-5(1,3)- benzenacyclotetradecaphane-12- yl)acetamide |
| 321 | 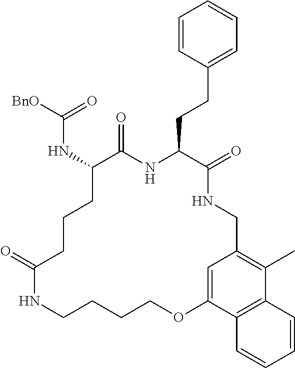 [SMILES: Cc1c2cc(c3ccccc13)OCCCCNC(=O)CCC[C@H](NC(=O)OCc1ccccc1)C(=O)N[C@@H](CCc1ccccc1)C(=O)NC2] | benzyl ((9S,12S)-5⁴-methyl-8,11,14- trioxo-9-phenethyl-4-oxa-7,10-diaza- 1(3,1)-piperidina-5(1,3)- naphthalenacyclotetradecaphane-12- yl)carbamate |
| 322 | 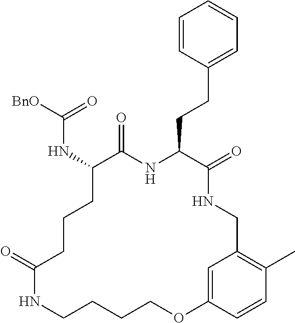 [SMILES: Cc1ccc2cc1CNC(=O)[C@H](CCc1ccccc1)NC(=O)[C@@H](NC(=O)OCc1ccccc1)CCCC(=O)NCCCCO2] | benzyl ((12S,15S)-1⁴-methyl-8,13,16- trioxo-15-phenethyl-2-oxa-7,14,17-triaza- 1(1,3)-benzenacyclooctadecaphane-12- yl)carbamate |
| 323 | 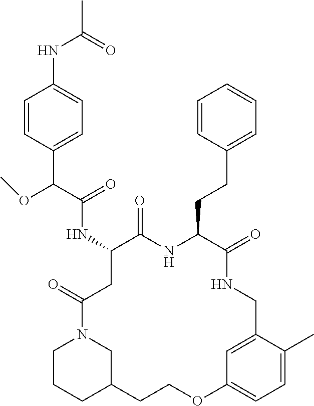 | 2-(4-acetamidophenyl)-2-methoxy-N- ((9S,12S)-5⁴-methyl-8,11,14-trioxo-9- phenethyl-4-oxa-7,10-diaza-1(3,1)- piperidina-5(1,3)- benzenacyclotetradecaphane-12- yl)acetamide |
| 324 | 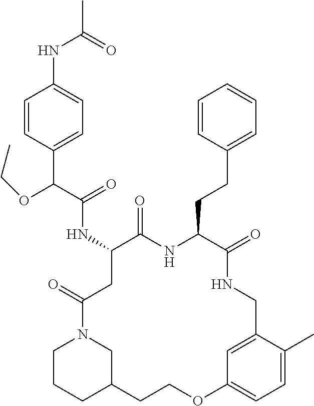 [SMILES: CCOC(C(=O)N[C@H]1CC(=O)N2CCCC(CCOc3ccc(C)c(c3)CNC(=O)[C@H](CCc3ccccc3)NC1=O)C2)c1ccc(NC(C)=O)cc1] | 2-(4-acetamidophenyl)-2-ethoxy-N- ((9S,12S)-5⁴-methyl-8,11,14-trioxo-9- phenethyl-4-oxa-7,10-diaza-1(3,1)- piperidina-5(1,3)- benzenacyclotetradecaphane-12- yl)acetamide |
| 325 | 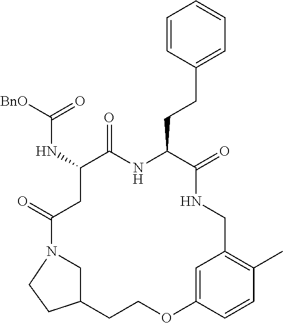 [SMILES: Cc1ccc2cc1CNC(=O)[C@H](CCc1ccccc1)NC(=O)[C@@H](NC(=O)OCc1ccccc1)CC(=O)N1CCC(CCO2)C1] | benzyl ((9S,12S)-5⁴-methyl-8,11,14- trioxo-9-phenethyl-4-oxa-7,10-diaza- 1(3,1)-pyrrolidina-5(1,3)- benzenacyclotetradecaphane-12- yl)carbamate |
| 326 | 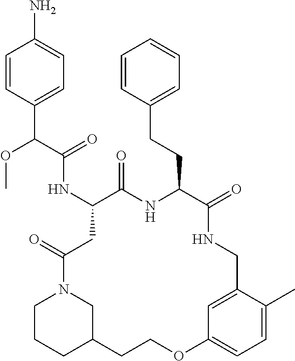 [SMILES: COC(C(=O)N[C@H]1CC(=O)N2CCCC(CCOc3ccc(C)c(c3)CNC(=O)[C@H](CCc3ccccc3)NC1=O)C2)c1ccc(N)cc1] | 2-(4-aminophenyl)-2-methoxy-N- ((9S,12S)-5⁴-methyl-8,11,14-trioxo-9- phenethyl-4-oxa-7,10-diaza-1(3,1)- piperidina-5(1,3)- benzenacyclotetradecaphane-12- yl)acetamide |
| 327 | 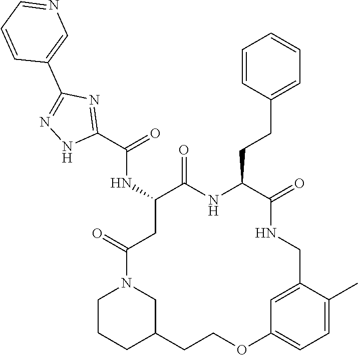 [SMILES: Cc1ccc2cc1CNC(=O)[C@H](CCc1ccccc1)NC(=O)[C@@H](NC(=O)c1nc(-c3cccnc3)n[nH]1)CC(=O)N1CCCC(CCO2)C1] | N-((9S,12R)-5⁴-methyl-8,11,14-trioxo-9- phenethyl-4-oxa-7,10-diaza-1(3,1)- piperidina-5(1,3)- benzenacyclotetradecaphane-12-yl)-2-(3- (pyridin-3-yl)-1H-1,2,4-triazol-5- yl)acetamide |
| 328 | 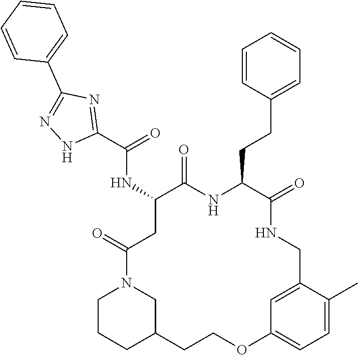 [SMILES: Cc1ccc2cc1CNC(=O)[C@H](CCc1ccccc1)NC(=O)[C@@H](NC(=O)c1nc(-c3ccccc3)n[nH]1)CC(=O)N1CCCC(CCO2)C1] | N-((9S,12S)-5⁴-methyl-8,11,14-trioxo-9- phenethyl-4-oxa-7,10-diaza-1(3,1)- piperidina-5(1,3)- benzenacyclotetradecaphane-12-yl)-2-(3- phenyl-1H-1,2,4-triazol-5-yl)acetamide |
| 329 | 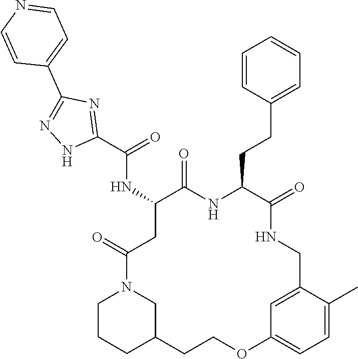 [SMILES: Cc1ccc2cc1CNC(=O)[C@H](CCc1ccccc1)NC(=O)[C@@H](NC(=O)c1nc(-c3ccncc3)n[nH]1)CC(=O)N1CCCC(CCO2)C1] | N-((9S,12S)-5⁴-methyl-8,11,14-trioxo-9- phenethyl-4-oxa-7,10-diaza-1(3,1)- piperidina-5(1,3)- benzenacyclotetradecaphane-12-yl)-2-(3- (pyridin-4-yl)-1H-1,2,4-triazol-5- yl)acetamide |
| 330 | 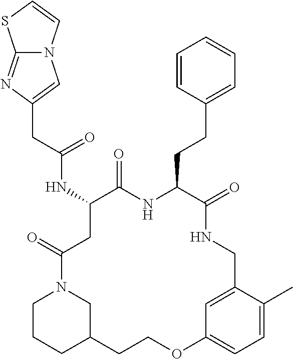 [SMILES: Cc1ccc2cc1CNC(=O)[C@H](CCc1ccccc1)NC(=O)[C@@H](NC(=O)Cc1cn3ccsc3n1)CC(=O)N1CCCC(CCO2)C1] | 2-(imidazo[2,1-b]thiazol-6-yl)-N- ((9S,12S)-5⁴-methyl-8,11,14-trioxo-9- phenethyl-4-oxa-7,10-diaza-1(3,1)- piperidina-5(1,3)- benzenacyclotetradecaphane-12- yl)acetamide |
| 331 | 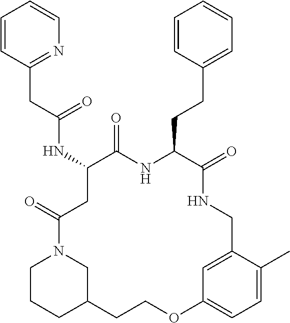 [SMILES: Cc1ccc2cc1CNC(=O)[C@H](CCc1ccccc1)NC(=O)[C@@H](NC(=O)Cc1ccccn1)CC(=O)N1CCCC(CCO2)C1] | N-((9S,12S)-5⁴-methyl-8,11,14-trioxo-9- phenethyl-4-oxa-7,10-diaza-1(3,1)- piperidina-5(1,3)- benzenacyclotetradecaphane-12-yl)-2- (pyridin-2-yl)acetamide |
| 332 | 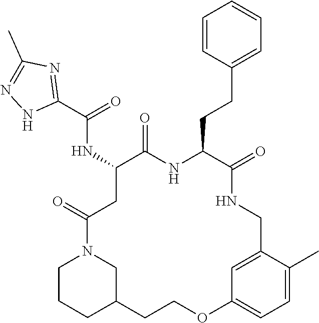 [SMILES: Cc1n[nH]c(C(=O)N[C@H]2CC(=O)N3CCCC(CCOc4ccc(C)c(c4)CNC(=O)[C@H](CCc4ccccc4)NC2=O)C3)n1] | 2-(3-methyl-1H-1,2,4-triazol-5-yl)-N- ((9S,12S)-5⁴-methyl-8,11,14-trioxo-9- phenethyl-4-oxa-7,10-diaza-1(3,1)- piperidina-5(1,3)- benzenacyclotetradecaphane-12- yl)acetamide |
| 333 | 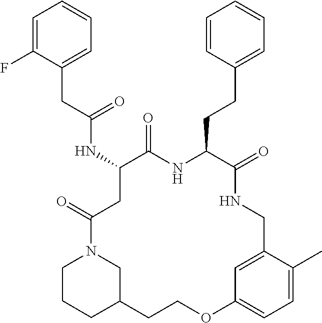 [SMILES: Cc1ccc2cc1CNC(=O)[C@H](CCc1ccccc1)NC(=O)[C@@H](NC(=O)Cc1ccccc1F)CC(=O)N1CCCC(CCO2)C1] | 2-(2-fluorophenyl)-N-((9S,12S)-5⁴-methyl- 8,11,14-trioxo-9-phenethyl-4-oxa-7,10- diaza-1(3,1)-piperidina-5(1,3)- benzenacyclotetradecaphane-12- yl)acetamide |
| 334 | 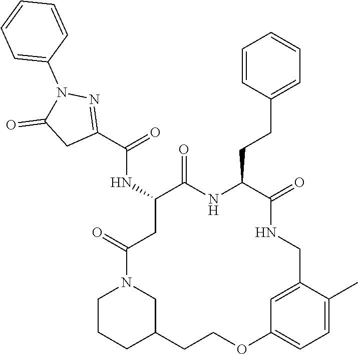 [SMILES: Cc1ccc2cc1CNC(=O)[C@H](CCc1ccccc1)NC(=O)[C@@H](NC(=O)C1=NN(c3ccccc3)C(=O)C1)CC(=O)N1CCCC(CCO2)C1] | N-((9S,12S)-5⁴-methyl-8,11,14-trioxo-9- phenethyl-4-oxa-7,10-diaza-1(3,1)- piperidina-5(1,3)- benzenacyclotetradecaphane-12-yl)-2-(5- oxo-1-phenyl-4,5-dihydro-1H-pyrazol-3- yl)acetamide |
| 335 | 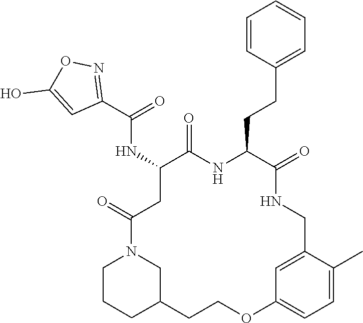 [SMILES: Cc1ccc2cc1CNC(=O)[C@H](CCc1ccccc1)NC(=O)[C@@H](NC(=O)c1cc(O)on1)CC(=O)N1CCCC(CCO2)C1] | 2-(5-hydroxyisoxazol-3-yl)-N-((9S,12S)- 5⁴-methyl-8,11,14-trioxo-9-phenethyl-4- oxa-7,10-diaza-1(3,1)-piperidina-5(1,3)- benzenacyclotetradecaphane-12- yl)acetamide |
| 336 | 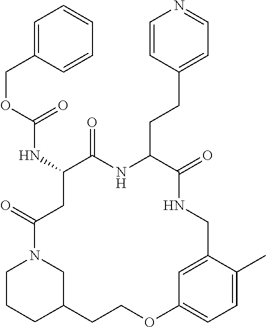 [SMILES: Cc1ccc2cc1CNC(=O)C(CCc1ccncc1)NC(=O)[C@@H](NC(=O)OCc1ccccc1)CC(=O)N1CCCC(CCO2)C1] | benzyl ((12S)-5⁴-methyl-8,11,14-trioxo-9- (2-(pyridin-4-yl)ethyl)-4-oxa-7,10-diaza- 1(3,1)-piperidina-5(1,3)- benzenacyclotetradecaphane-12- yl)carbamate |
| 337 | 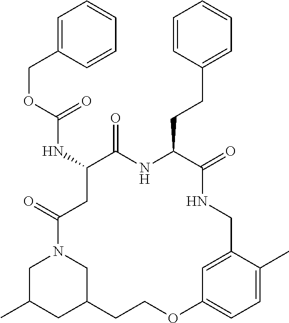 | benzyl ((9S,12S)-15,5⁴-dimethyl-8,11,14- trioxo-9-phenethyl-4-oxa-7,10-diaza- 1(3,1)-piperidina-5(1,3)- benzenacyclotetradecaphane-12- yl)carbamate |
| 338 | 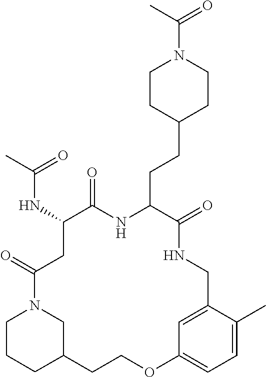 [SMILES: CC(=O)N[C@H]1CC(=O)N2CCCC(CCOc3ccc(C)c(c3)CNC(=O)C(CCC3CCN(C(C)=O)CC3)NC1=O)C2] | N-((12S)-9-(2-(1-acetylpiperidin-4- yl)ethyl)-5⁴-methyl-8,11,14-trioxo-4-oxa- 7,10-diaza-1(3,1)-piperidina-5(1,3)- benzenacyclotetradecaphane-12- yl)acetamide |
| 339 | 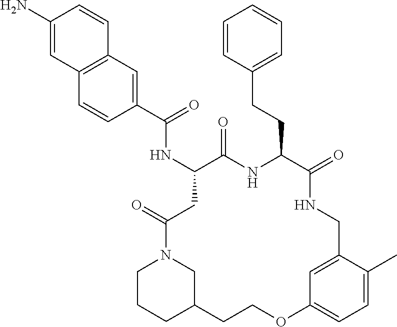 [SMILES: Cc1ccc2cc1CNC(=O)[C@H](CCc1ccccc1)NC(=O)[C@@H](NC(=O)c1ccc3cc(N)ccc3c1)CC(=O)N1CCCC(CCO2)C1] | 6-amino-N-((9S,12S)-5⁴-methyl-8,11,14- trioxo-9-phenethyl-4-oxa-7,10-diaza- 1(3,1)-piperidina-5(1,3)- benzenacyclotetradecaphane-12-yl)-2- naphthamide |
| 340 | 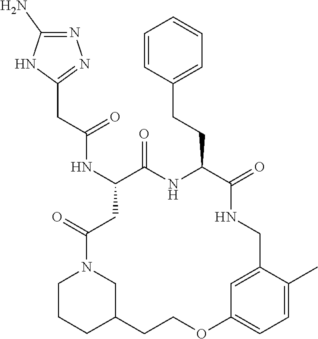 [SMILES: Cc1ccc2cc1CNC(=O)[C@H](CCc1ccccc1)NC(=O)[C@@H](NC(=O)Cc1nnc(N)[nH]1)CC(=O)N1CCCC(CCO2)C1] | 2-(5-amino-1H-1,2,4-triazol-3-yl)-N- ((9S,12S)-5⁴-methyl-8,11,14-trioxo-9- phenethyl-4-oxa-7,10-diaza-1(3,1)- piperidina-5(1,3)- benzenacyclotetradecaphane-12- yl)acetamide |
| 341 | 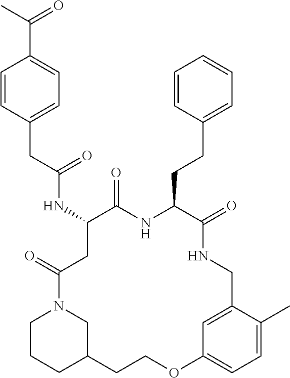 [SMILES: CC(=O)c1ccc(CC(=O)N[C@H]2CC(=O)N3CCCC(CCOc4ccc(C)c(c4)CNC(=O)[C@H](CCc4ccccc4)NC2=O)C3)cc1] | 2-(4-acetylphenyl)-N-((9S,12S)-5⁴-methyl- 8,11,14-trioxo-9-phenethyl-4-oxa-7,10- diaza-1(3,1)-piperidina-5(1,3)- benzenacyclotetradecaphane-12- yl)acetamide |
| 342 | 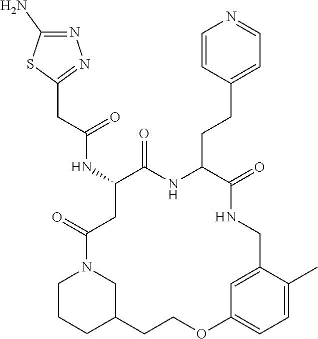 [SMILES: Cc1ccc2cc1CNC(=O)C(CCc1ccncc1)NC(=O)[C@@H](NC(=O)Cc1nnc(N)s1)CC(=O)N1CCCC(CCO2)C1] | 2-(5-amino-1,3,4-thiadiazol-2-yl)-N- ((12S)-5⁴-methyl-8,11,14-trioxo-9-(2- (pyridin-4-yl)ethyl)-4-oxa-7,10-diaza- 1(3,1)-piperidina-5(1,3)- benzenacyclotetradecaphane-12- yl)acetamide |
| 343 | 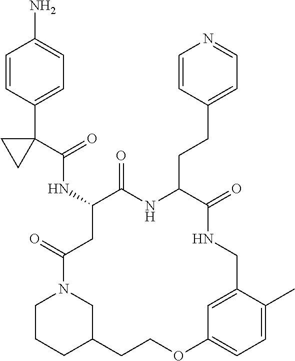 [SMILES: Cc1ccc2cc1CNC(=O)C(CCc1ccncc1)NC(=O)[C@@H](NC(=O)C1(c3ccc(N)cc3)CC1)CC(=O)N1CCCC(CCO2)C1] | 1-(4-aminophenyl)-N-((12S)-5⁴-methyl- 8,11,14-trioxo-9-(2-(pyridin-4-yl)ethyl)-4- oxa-7,10-diaza-1(3,1)-piperidina-5(1,3)- benzenacyclotetradecaphane-12- yl)cyclopropane-1-carboxamide |
| 344 | 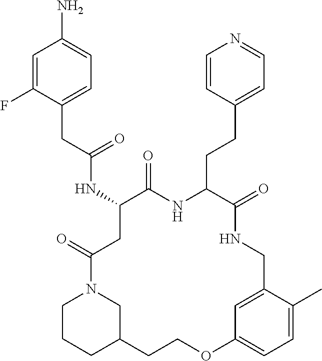 [SMILES: Cc1ccc2cc1CNC(=O)C(CCc1ccncc1)NC(=O)[C@@H](NC(=O)Cc1ccc(N)cc1F)CC(=O)N1CCCC(CCO2)C1] | 2-(4-amino-2-fluorophenyl)-N-((12S)-5⁴- methyl-8,11,14-trioxo-9-(2-(pyridin-4- yl)ethyl)-4-oxa-7,10-diaza-1(3,1)- piperidina-5(1,3)- benzenacyclotetradecaphane-12- yl)acetamide |
| 345 | 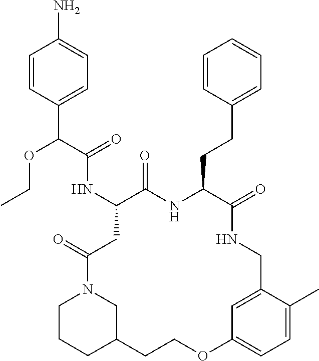 [SMILES: CCOC(C(=O)N[C@H]1CC(=O)N2CCCC(CCOc3ccc(C)c(c3)CNC(=O)[C@H](CCc3ccccc3)NC1=O)C2)c1ccc(N)cc1] | 2-(4-aminophenyl)-2-ethoxy-N-((9S,12S)- 5⁴-methyl-8,11,14-trioxo-9-phenethyl-4- oxa-7,10-diaza-1(3,1)-piperidina-5(1,3)- benzenacyclotetradecaphane-12- yl)acetamide |
| 346 | 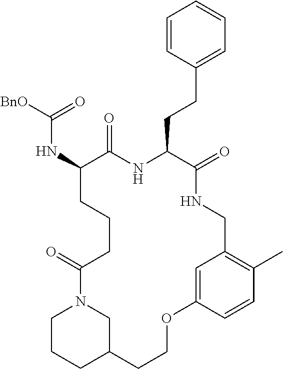 [SMILES: Cc1ccc2cc1CNC(=O)[C@H](CCc1ccccc1)NC(=O)[C@H](NC(=O)OCc1ccccc1)CCCC(=O)N1CCCC(CCO2)C1] | benzyl ((9S,12R)-5⁴-methyl-8,11,16- trioxo-9-phenethyl-4-oxa-7,10-diaza- 1(3,1)-piperidina-5(1,3)- benzenacyclohexadecaphane-12- yl)carbamate |
| 347 | 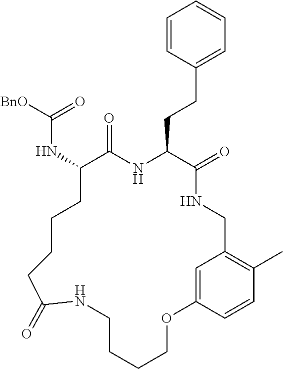 [SMILES: Cc1ccc2cc1CNC(=O)[C@H](CCc1ccccc1)NC(=O)[C@@H](NC(=O)OCc1ccccc1)CCCCC(=O)NCCCCO2] | benzyl ((13S,16S)-1⁴-methyl-8,14,17- trioxo-16-phenethyl-2-oxa-7,15,18-triaza- 1(1,3)-benzenacyclononadecaphane-13- yl)carbamate |
| 348 | 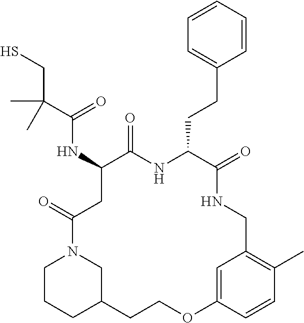 [SMILES: Cc1ccc2cc1CNC(=O)[C@@H](CCc1ccccc1)NC(=O)[C@H](NC(=O)C(C)(C)CS)CC(=O)N1CCCC(CCO2)C1] | 3-mercapto-2,2-dimethyl-N-((9S,12S)-5⁴- methyl-8,11,14-trioxo-9-phenethyl-4-oxa- 7,10-diaza-1(3,1)-piperidina-5(1,3)- benzenacyclotetradecaphane-12- yl)propanamide |
| 349 | 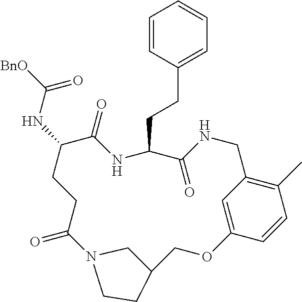 [SMILES: Cc1ccc2cc1CNC(=O)[C@H](CCc1ccccc1)NC(=O)[C@@H](NC(=O)OCc1ccccc1)CCC(=O)N1CCC(CO2)C1] | benzyl ((8S,11S)-4⁴-methyl-7,10,14- trioxo-8-phenethyl-3-oxa-6,9-diaza-1(3,1)- pyrrolidina-4(1,3)- benzenacyclotetradecaphane-11- yl)carbamate |
| 350 | 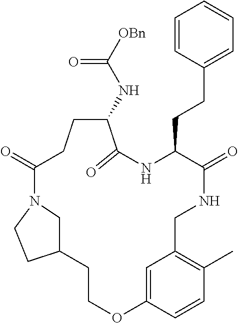 [SMILES: Cc1ccc2cc1CNC(=O)[C@H](CCc1ccccc1)NC(=O)[C@@H](NC(=O)OCc1ccccc1)CCC(=O)N1CCC(CCO2)C1] | benzyl ((9S,12S)-5⁴-methyl-8,11,15- trioxo-9-phenethyl-4-oxa-7,10-diaza- 1(3,1)-pyrrolidina-5(1,3)- benzenacyclopentadecaphane-12- yl)carbamate |
| 351 | 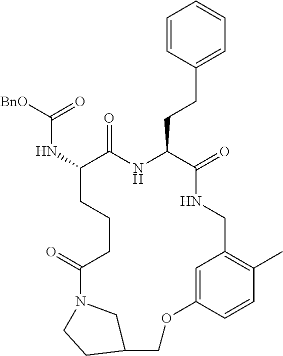 [SMILES: Cc1ccc2cc1CNC(=O)[C@H](CCc1ccccc1)NC(=O)[C@@H](NC(=O)OCc1ccccc1)CCCC(=O)N1CCC(CO2)C1] | benzyl ((8S,11S)-4⁴-methyl-7,10,15- trioxo-8-phenethyl-3-oxa-6,9-diaza-1(3,1)- pyrrolidina-4(1,3)- benzenacyclopentadecaphane-11- yl)carbamate |
| 352 | 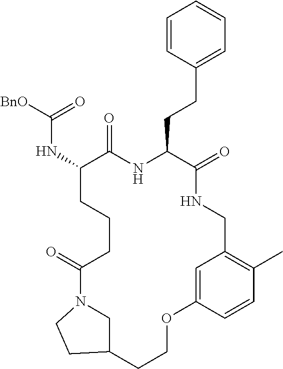 [SMILES: Cc1ccc2cc1CNC(=O)[C@H](CCc1ccccc1)NC(=O)[C@@H](NC(=O)OCc1ccccc1)CCCC(=O)N1CCC(CCO2)C1] | benzyl ((9S,12S)-5⁴-methyl-8,11,16- trioxo-9-phenethyl-4-oxa-7,10-diaza- 1(3,1)-pyrrolidina-5(1,3)- benzenacyclohexadecaphane-12- yl)carbamate |
| 353 | 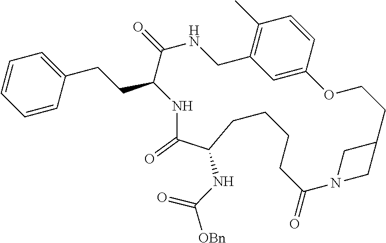 [SMILES: Cc1ccc2cc1CNC(=O)[C@H](CCc1ccccc1)NC(=O)[C@@H](NC(=O)OCc1ccccc1)CCCCC(=O)N1CC(CCO2)C1] | benzyl ((9S,12S)-5⁴-methyl-8,11,17- trioxo-9-phenethyl-4-oxa-7,10-diaza- 1(3,1)-azetidina-5(1,3)- benzenacycloheptadecaphane-12- yl)carbamate |
| 354 | 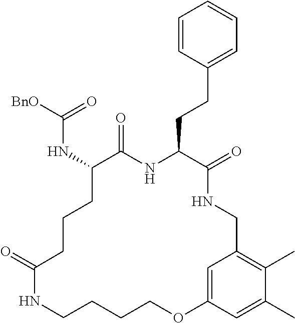 [SMILES: Cc1cc2cc(c1C)CNC(=O)[C@H](CCc1ccccc1)NC(=O)[C@@H](NC(=O)OCc1ccccc1)CCCC(=O)NCCCCO2] | benzyl ((12S,15S)-1⁴,1⁵-dimethyl-8,13,16- trioxo-15-phenethyl-2-oxa-7,14,17-triaza- 1(1,3)-benzenacyclooctadecaphane-12- yl)carbamate |
| 355 | 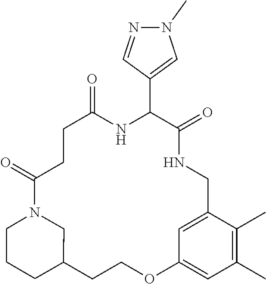 [SMILES: Cc1cc2cc(c1C)CNC(=O)C(c1cnn(C)c1)NC(=O)CCC(=O)N1CCCC(CCO2)C1] | 5⁴,5⁵-dimethyl-9-(1-methyl-1H-pyrazol-4- yl)-4-oxa-7,10-diaza-1(3,1)-piperidina- 5(1,3)-benzenacyclotetradecaphane- 8,11,14-trione |
| 356 | 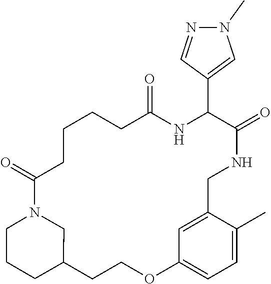 [SMILES: Cc1ccc2cc1CNC(=O)C(c1cnn(C)c1)NC(=O)CCCCC(=O)N1CCCC(CCO2)C1] | 5⁴-methyl-9-(1-methyl-1H-pyrazol-4-yl)-4- oxa-7,10-diaza-1(3,1)-piperidina-5(1,3)- benzenacyclohexadecaphane-8,11,16- trione |
| 358 | 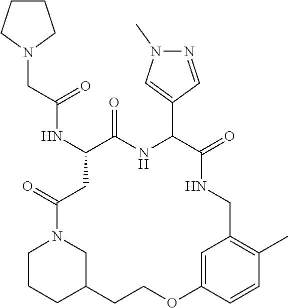 [SMILES: Cc1ccc2cc1CNC(=O)C(c1cnn(C)c1)NC(=O)[C@@H](NC(=O)CN1CCCC1)CC(=O)N1CCCC(CCO2)C1] | N-((12S)-5⁴-methyl-9-(1-methyl-1H- pyrazol-4-yl)-8,11,14-trioxo-4-oxa-7,10- diaza-1(3,1)-piperidina-5(1,3)- benzenacyclotetradecaphane-12-yl)-2- (pyrrolidin-1-yl)acetamide |
| 359 | 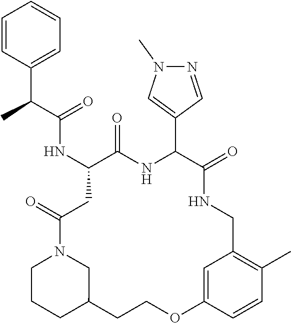 [SMILES: Cc1ccc2cc1CNC(=O)C(c1cnn(C)c1)NC(=O)[C@@H](NC(=O)[C@@H](C)c1ccccc1)CC(=O)N1CCCC(CCO2)C1] | (2S)-N-((12S)-5⁴-methyl-9-(1-methyl-1H- pyrazol-4-yl)-8,11,14-trioxo-4-oxa-7,10- diaza-1(3,1)-piperidina-5(1,3)- benzenacyclotetradecaphane-12-yl)-2- phenylpropanamide |
| 360 | 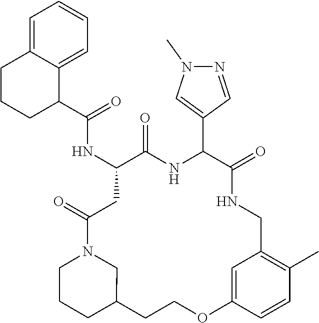 [SMILES: Cc1ccc2cc1CNC(=O)C(c1cnn(C)c1)NC(=O)[C@@H](NC(=O)C1CCCc3ccccc31)CC(=O)N1CCCC(CCO2)C1] | N-((12S)-5⁴-methyl-9-(1-methyl-1H- pyrazol-4-yl)-8,11,14-trioxo-4-oxa-7,10- diaza-1(3,1)-piperidina-5(1,3)- benzenacyclotetradecaphane-12-yl)- 1,2,3,4-tetrahydronaphthalene-1- carboxamide |
| 361 | 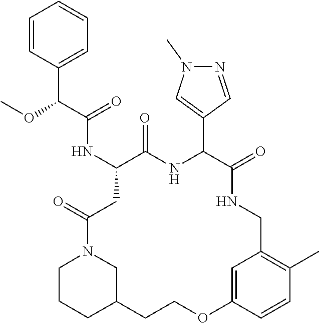 | (2R)-2-methoxy-N-((12S)-5⁴-methyl-9-(1- methyl-1H-pyrazol-4-yl)-8,11,14-trioxo-4- oxa-7,10-diaza-1(3,1)-piperidina-5(1,3)- benzenacyclotetradecaphane-12-yl)-2- phenylacetamide |
| 362 | 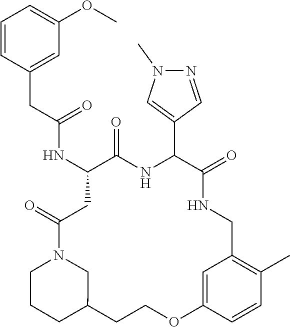 [SMILES: COc1cccc(CC(=O)N[C@H]2CC(=O)N3CCCC(CCOc4ccc(C)c(c4)CNC(=O)C(c4cnn(C)c4)NC2=O)C3)c1] | 2-(3-methoxyphenyl)-N-((12S)-5⁴-methyl- 9-(1-methyl-1H-pyrazol-4-yl)-8,11,14- trioxo-4-oxa-7,10-diaza-1(3,1)-piperidina- 5(1,3)-benzenacyclotetradecaphane-12- yl)acetamide |
| 363 | 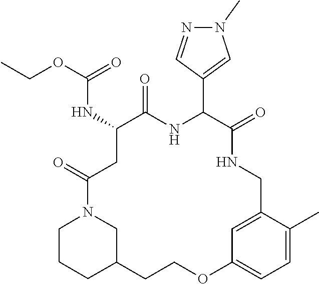 [SMILES: CCOC(=O)N[C@H]1CC(=O)N2CCCC(CCOc3ccc(C)c(c3)CNC(=O)C(c3cnn(C)c3)NC1=O)C2] | Ethyl ((12S)-5⁴-methyl-9-(1-methyl-1H- pyrazol-4-yl)-8,11,14-trioxo-4-oxa-7,10- diaza-1(3,1)-piperidina-5(1,3)- benzenacyclotetradecaphane-12- yl)carbamate |
| 364 | 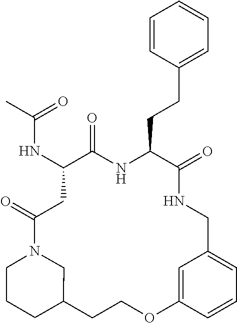 [SMILES: CC(=O)N[C@H]1CC(=O)N2CCCC(CCOc3cccc(c3)CNC(=O)[C@H](CCc3ccccc3)NC1=O)C2] | N-((9S,12S)-8,11,14-trioxo-9-phenethyl-4- oxa-7,10-diaza-1(3,1)-piperidina-5(1,3)- benzenacyclotetradecaphane-12- yl)acetamide |
| 369 | 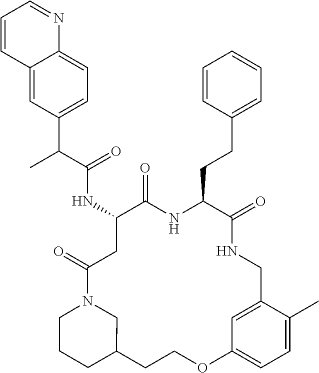 [SMILES: Cc1ccc2cc1CNC(=O)[C@H](CCc1ccccc1)NC(=O)[C@@H](NC(=O)C(C)c1ccc3ncccc3c1)CC(=O)N1CCCC(CCO2)C1] | N-((9S,12S)-5⁴-methyl-8,11,14-trioxo-9- phenethyl-4-oxa-7,10-diaza-1(3,1)- piperidina-5(1,3)- benzenacyclotetradecaphane-12-yl)-2- (quinolin-6-yl)propanamide |
| 371 | 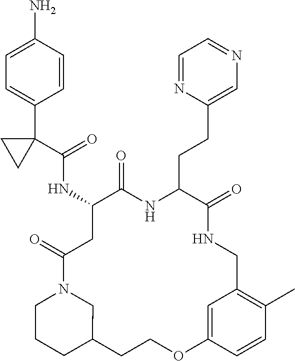 [SMILES: Cc1ccc2cc1CNC(=O)C(CCc1cnccn1)NC(=O)[C@@H](NC(=O)C1(c3ccc(N)cc3)CC1)CC(=O)N1CCCC(CCO2)C1] | 1-(4-aminophenyl)-N-((9S,12S)-5⁴- methyl-8,11,14-trioxo-9-(2-(pyrazin-2- yl)ethyl)-4-oxa-7,10-diaza-1(3,1)- piperidina-5(1,3)- benzenacyclotetradecaphane-12- yl)cyclopropane-1-carboxamide |
| 372 | 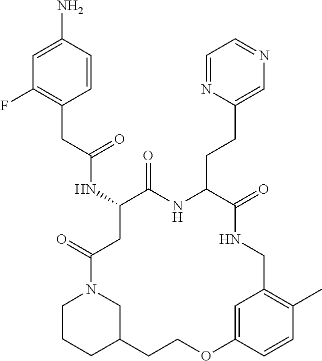 [SMILES: Cc1ccc2cc1CNC(=O)C(CCc1cnccn1)NC(=O)[C@@H](NC(=O)Cc1ccc(N)cc1F)CC(=O)N1CCCC(CCO2)C1] | 2-(4-amino-2-fluorophenyl)-N-((9S,12S)- 5⁴-methyl-8,11,14-trioxo-9-(2-(pyrazin-2- yl)ethyl)-4-oxa-7,10-diaza-1(3,1)- piperidina-5(1,3)- benzenacyclotetradecaphane-12- yl)acetamide |
| 373 | 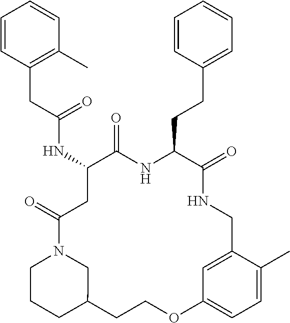 [SMILES: Cc1ccc2cc1CNC(=O)[C@H](CCc1ccccc1)NC(=O)[C@@H](NC(=O)Cc1ccccc1C)CC(=O)N1CCCC(CCO2)C1] | N-((9S,12S)-5⁴-methyl-8,11,14-trioxo-9- phenethyl-4-oxa-7,10-diaza-1(3,1)- piperidina-5(1,3)- benzenacyclotetradecaphane-12-yl)-2-(o- tolyl)acetamide |
| 387 | 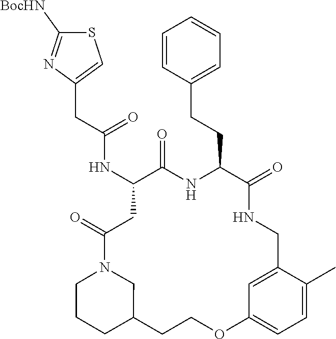 [SMILES: Cc1ccc2cc1CNC(=O)[C@H](CCc1ccccc1)NC(=O)[C@@H](NC(=O)Cc1csc(NC(=O)OC(C)(C)C)n1)CC(=O)N1CCCC(CCO2)C1] | tert-butyl (4-(2-(((9S,12S)-5⁴-methyl- 8,11,14-trioxo-9-phenethyl-4-oxa-7,10- diaza-1(3,1)-piperidina-5(1,3)- benzenacyclotetradecaphane-12- yl)amino)-2-oxoethyl)thiazol-2- yl)carbamate |
| 388 | 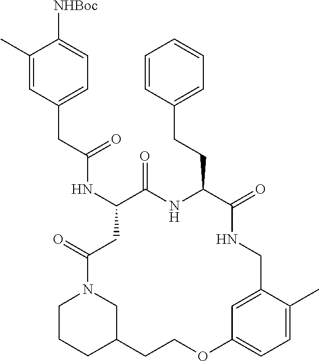 | tert-butyl (2-methyl-4-(2-(((9S,12S)-5⁴- methyl-8,11,14-trioxo-9-phenethyl-4-oxa- 7,10-diaza-1(3,1)-piperidina-5(1,3)- benzenacyclotetradecaphane-12- yl)amino)-2-oxoethyl)phenyl)carbamate |
| 389 | 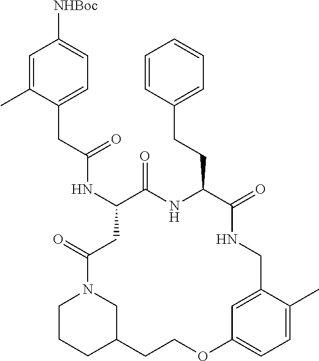 | tert-butyl (3-methyl-4-(2-(((9S,12S)-5⁴- methyl-8,11,14-trioxo-9-phenethyl-4-oxa- 7,10-diaza-1(3,1)-piperidina-5(1,3)- benzenacyclotetradecaphane-12- yl)amino)-2-oxoethyl)phenyl)carbamate |
| 390 | 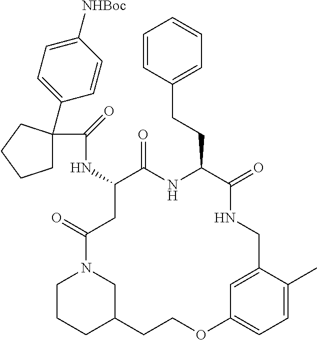 [SMILES: Cc1ccc2cc1CNC(=O)[C@H](CCc1ccccc1)NC(=O)[C@@H](NC(=O)C1(c3ccc(NC(=O)OC(C)(C)C)cc3)CCCC1)CC(=O)N1CCCC(CCO2)C1] | tert-butyl (4-(1-(((9S,12S)-5⁴-methyl- 8,11,14-trioxo-9-phenethyl-4-oxa-7,10- diaza-1(3,1)-piperidina-5(1,3)- benzenacyclotetradecaphane-12- yl)carbamoyl)cyclopentyl)phenyl) carbamate |
| 391 | 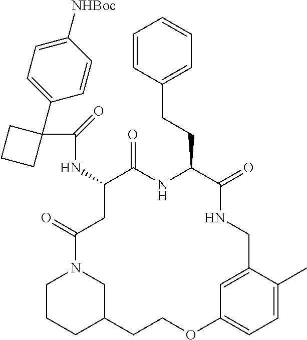 [SMILES: Cc1ccc2cc1CNC(=O)[C@H](CCc1ccccc1)NC(=O)[C@@H](NC(=O)C1(c3ccc(NC(=O)OC(C)(C)C)cc3)CCC1)CC(=O)N1CCCC(CCO2)C1] | tert-butyl (4-(1-(((9S,12S)-5⁴-methyl- 8,11,14-trioxo-9-phenethyl-4-oxa-7,10- diaza-1(3,1)-piperidina-5(1,3)- benzenacyclotetradecaphane-12- yl)carbamoyl)cyclobutyl)phenyl)carbamate |
| 392 | 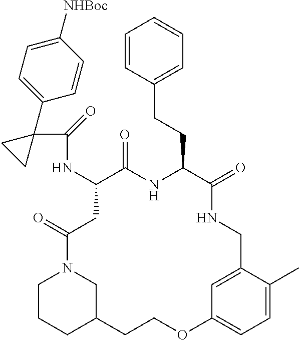 [SMILES: Cc1ccc2cc1CNC(=O)[C@H](CCc1ccccc1)NC(=O)[C@@H](NC(=O)C1(c3ccc(NC(=O)OC(C)(C)C)cc3)CC1)CC(=O)N1CCCC(CCO2)C1] | tert-butyl (4-(1-(((9S,12S)-5⁴-methyl- 8,11,14-trioxo-9-phenethyl-4-oxa-7,10- diaza-1(3,1)-piperidina-5(1,3)- benzenacyclotetradecaphane-12- yl)carbamoyl)cyclopropyl)phenyl)carbamate |
| 393 | 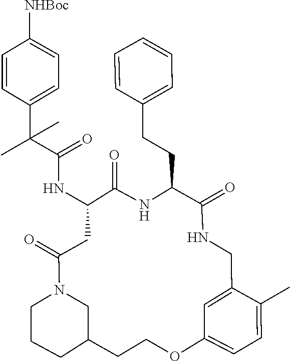 [SMILES: Cc1ccc2cc1CNC(=O)[C@H](CCc1ccccc1)NC(=O)[C@@H](NC(=O)C(C)(C)c1ccc(NC(=O)OC(C)(C)C)cc1)CC(=O)N1CCCC(CCO2)C1] | tert-butyl (4-(2-methyl-1-(((9S,12S)-5⁴- methyl-8,11,14-trioxo-9-phenethyl-4-oxa- 7,10-diaza-1(3,1)-piperidina-5(1,3)- benzenacyclotetradecaphane-12- yl)amino)-1-oxopropan-2- yl)phenyl)carbamate |
| 394 | 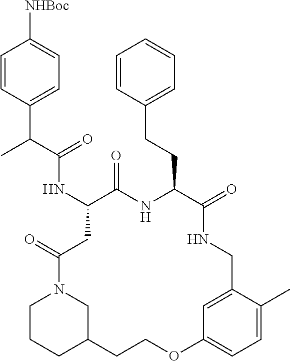 [SMILES: Cc1ccc2cc1CNC(=O)[C@H](CCc1ccccc1)NC(=O)[C@@H](NC(=O)C(C)c1ccc(NC(=O)OC(C)(C)C)cc1)CC(=O)N1CCCC(CCO2)C1] | 2-(4-aminophenyl)-N-((9S,12S)-5⁴- methyl-8,11,14-trioxo-9-phenethyl-4-oxa- 7,10-diaza-1(3,1)-piperidina-5(1,3)- benzenacyclotetradecaphane-12- yl)propanamide |
| 395 | 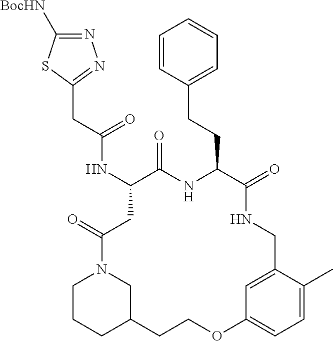 [SMILES: Cc1ccc2cc1CNC(=O)[C@H](CCc1ccccc1)NC(=O)[C@@H](NC(=O)Cc1nnc(NC(=O)OC(C)(C)C)s1)CC(=O)N1CCCC(CCO2)C1] | tert-butyl (5-(2-(((9S,12S)-5⁴-methyl- 8,11,14-trioxo-9-phenethyl-4-oxa-7,10- diaza-1(3,1)-piperidina-5(1,3)- benzenacyclotetradecaphane-12- yl)amino)-2-oxoethyl)-1,3,4-thiadiazol-2- yl)carbamate |
| 396 | 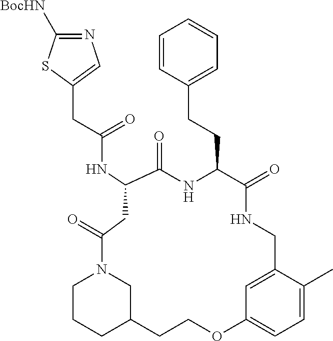 [SMILES: Cc1ccc2cc1CNC(=O)[C@H](CCc1ccccc1)NC(=O)[C@@H](NC(=O)Cc1cnc(NC(=O)OC(C)(C)C)s1)CC(=O)N1CCCC(CCO2)C1] | tert-butyl (5-(2-(((9S,12S)-5⁴-methyl- 8,11,14-trioxo-9-phenethyl-4-oxa-7,10- diaza-1(3,1)-piperidina-5(1,3)- benzenacyclotetradecaphane-12- yl)amino)-2-oxoethyl)thiazol-2- yl)carbamate |
| 402 | 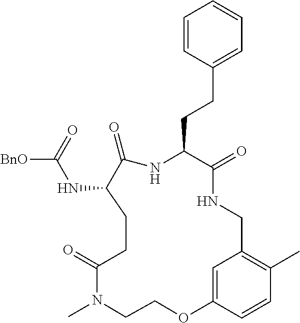 [SMILES: Cc1ccc2cc1CNC(=O)[C@H](CCc1ccccc1)NC(=O)[C@@H](NC(=O)OCc1ccccc1)CCC(=O)N(C)CCO2] | benzyl ((9S,12S)-1⁴,5-dimethyl-6,10,13- trioxo-12-phenethyl-2-oxa-5,11,14-triaza- 1(1,3)-benzenacyclopentadecaphane-9- yl)carbamate |
| 405 | 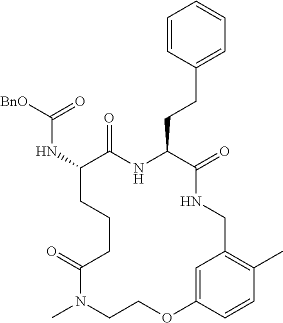 [SMILES: Cc1ccc2cc1CNC(=O)[C@H](CCc1ccccc1)NC(=O)[C@@H](NC(=O)OCc1ccccc1)CCCC(=O)N(C)CCO2] | benzyl ((10S,13S)-1⁴,5-dimethyl-6,11,14- trioxo-13-phenethyl-2-oxa-5,12,15-triaza- 1(1,3)-benzenacyclohexadecaphane-10- yl)carbamate |
| 419 | 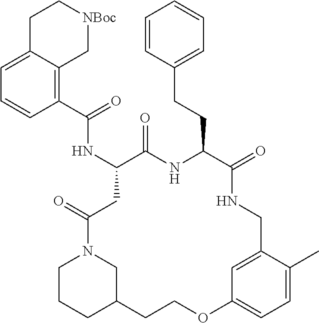 [SMILES: Cc1ccc2cc1CNC(=O)[C@H](CCc1ccccc1)NC(=O)[C@@H](NC(=O)c1cccc3c1CN(C(=O)OC(C)(C)C)CC3)CC(=O)N1CCCC(CCO2)C1] | tert-butyl 8-(((9S,12S)-5⁴-methyl-8,11,14- trioxo-9-phenethyl-4-oxa-7,10-diaza- 1(3,1)-piperidina-5(1,3)- benzenacyclotetradecaphane-12- yl)carbamoyl)-3,4-dihydroisoquinoline- 2(1H)-carboxylate |
| 420 | 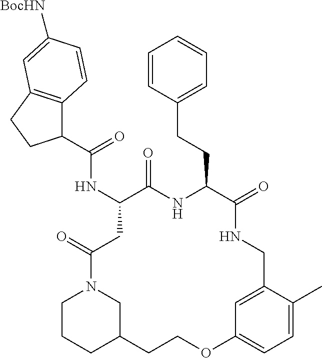 [SMILES: Cc1ccc2cc1CNC(=O)[C@H](CCc1ccccc1)NC(=O)[C@@H](NC(=O)C1CCc3cc(NC(=O)OC(C)(C)C)ccc31)CC(=O)N1CCCC(CCO2)C1] | tert-butyl (1-(((9S,12S)-5⁴-methyl-8,11,14- trioxo-9-phenethyl-4-oxa-7,10-diaza- 1(3,1)-piperidina-5(1,3)- benzenacyclotetradecaphane-12- yl)carbamoyl)-2,3-dihydro-1H-inden-5- yl)carbamate |
| 421 | 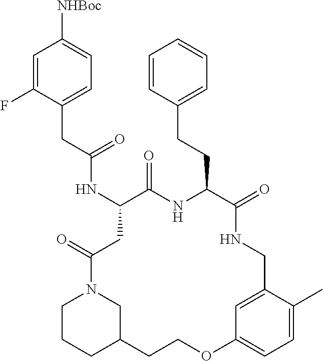 [SMILES: Cc1ccc2cc1CNC(=O)[C@H](CCc1ccccc1)NC(=O)[C@@H](NC(=O)Cc1ccc(NC(=O)OC(C)(C)C)cc1F)CC(=O)N1CCCC(CCO2)C1] | tert-butyl (3-fluoro-4-(2-(((9S,12S)-5⁴- methyl-8,11,14-trioxo-9-phenethyl-4-oxa- 7,10-diaza-1(3,1)-piperidina-5(1,3)- benzenacyclotetradecaphane-12- yl)amino)-2-oxoethyl)phenyl)carbamate |
| 422 | 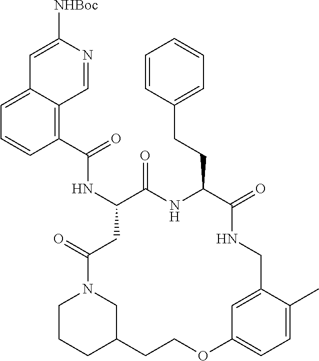 [SMILES: Cc1ccc2cc1CNC(=O)[C@H](CCc1ccccc1)NC(=O)[C@@H](NC(=O)c1cccc3cc(NC(=O)OC(C)(C)C)ncc13)CC(=O)N1CCCC(CCO2)C1] | tert-butyl (8-(((9S,12S)-5⁴-methyl-8,11,14- trioxo-9-phenethyl-4-oxa-7,10-diaza- 1(3,1)-piperidina-5(1,3)- benzenacyclotetradecaphane-12- yl)carbamoyl)isoquinolin-3-yl)carbamate |
| 423 | 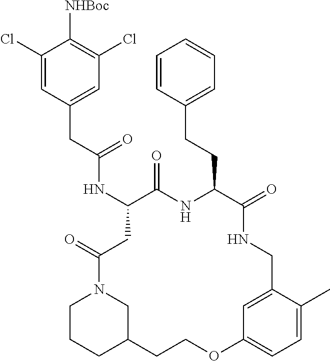 [SMILES: Cc1ccc2cc1CNC(=O)[C@H](CCc1ccccc1)NC(=O)[C@@H](NC(=O)Cc1cc(Cl)c(NC(=O)OC(C)(C)C)c(Cl)c1)CC(=O)N1CCCC(CCO2)C1] | tert-butyl (2,6-dichloro-4-(2-(((9S,12S)-5⁴- methyl-8,11,14-trioxo-9-phenethyl-4-oxa- 7,10-diaza-1(3,1)-piperidina-5(1,3)- benzenacyclotetradecaphane-12- yl)amino)-2-oxoethyl)phenyl)carbamate |
| 429 | 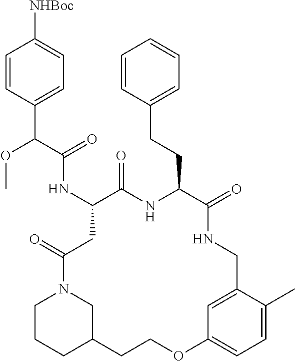 [SMILES: COC(C(=O)N[C@H]1CC(=O)N2CCCC(CCOc3ccc(C)c(c3)CNC(=O)[C@H](CCc3ccccc3)NC1=O)C2)c1ccc(NC(=O)OC(C)(C)C)cc1] | tert-butyl (4-(1-methoxy-2-(((9S,12S)-5⁴- methyl-8,11,14-trioxo-9-phenethyl-4-oxa- 7,10-diaza-1(3,1)-piperidina-5(1,3)- benzenacyclotetradecaphane-12- yl)amino)-2-oxoethyl)phenyl)carbamate |
| 437 | 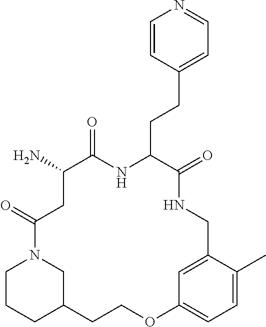 [SMILES: Cc1ccc2cc1CNC(=O)C(CCc1ccncc1)NC(=O)[C@@H](N)CC(=O)N1CCCC(CCO2)C1] | ((12S)-12-amino-5⁴-methyl-8,11,14- trioxo-9-(2-(pyridin-4-yl)ethyl)-4-oxa-7,10- diaza-1(3,1)-piperidina-5(1,3)- benzenacyclotetradecaphane- |
| 438 | 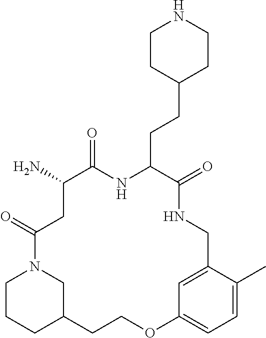 [SMILES: Cc1ccc2cc1CNC(=O)C(CCC1CCNCC1)NC(=O)[C@@H](N)CC(=O)N1CCCC(CCO2)C1] | N-((12S)-12-amino-9-(2-(1- acetylpiperidin-4-yl)ethyl)-5⁴-methyl- 8,11,14-trioxo-4-oxa-7,10-diaza-1(3,1)- piperidina-5(1,3)- benzenacyclotetradecaphane |
| 439 | 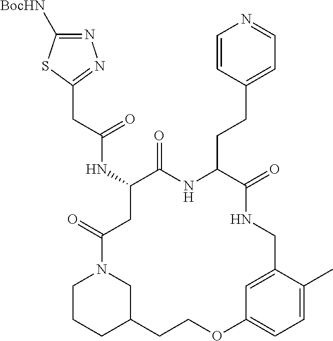 [SMILES: Cc1ccc2cc1CNC(=O)C(CCc1ccncc1)NC(=O)[C@@H](NC(=O)Cc1nnc(NC(=O)OC(C)(C)C)s1)CC(=O)N1CCCC(CCO2)C1] | tert-butyl (5-(2-(((12S)-5⁴-methyl-8,11,14- trioxo-9-(2-(pyridin-4-yl)ethyl)-4-oxa-7,10- diaza-1(3,1)-piperidina-5(1,3)- benzenacyclotetradecaphane-12- yl)amino)-2-oxoethyl)-1,3,4-thiadiazol-2- yl)carbamate |
| 440 | 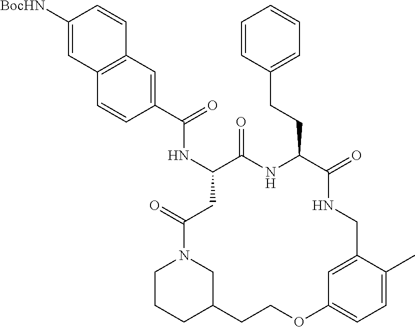 [SMILES: Cc1ccc2cc1CNC(=O)[C@H](CCc1ccccc1)NC(=O)[C@@H](NC(=O)c1ccc3cc(NC(=O)OC(C)(C)C)ccc3c1)CC(=O)N1CCCC(CCO2)C1] | tert-butyl (6-(((9S,12S)-5⁴-methyl-8,11,14- trioxo-9-phenethyl-4-oxa-7,10-diaza- 1(3,1)-piperidina-5(1,3)- benzenacyclotetradecaphane-12- yl)carbamoyl)naphthalen-2-yl)carbamate |
| 441 | 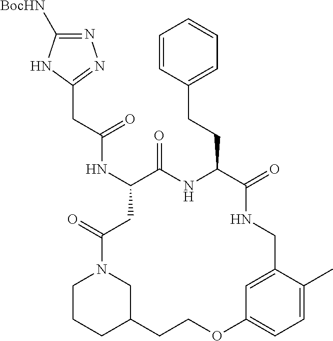 [SMILES: Cc1ccc2cc1CNC(=O)[C@H](CCc1ccccc1)NC(=O)[C@@H](NC(=O)Cc1nnc(NC(=O)OC(C)(C)C)[nH]1)CC(=O)N1CCCC(CCO2)C1] | tert-butyl (5-(2-(((9S,12S)-5⁴-methyl- 8,11,14-trioxo-9-phenethyl-4-oxa-7,10- diaza-1(3,1)-piperidina-5(1,3)- benzenacyclotetradecaphane-12- yl)amino)-2-oxoethyl)-4H-1,2,4-triazol-3- yl)carbamate |
| 442 | 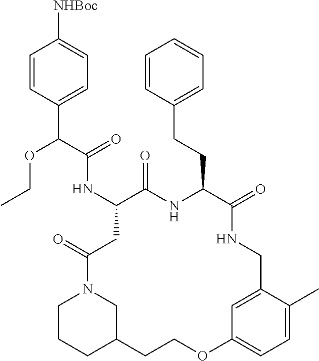 [SMILES: CCOC(C(=O)N[C@H]1CC(=O)N2CCCC(CCOc3ccc(C)c(c3)CNC(=O)[C@H](CCc3ccccc3)NC1=O)C2)c1ccc(NC(=O)OC(C)(C)C)cc1] | tert-butyl (4-(1-ethoxy-2-(((9S,12S)-5⁴- methyl-8,11,14-trioxo-9-phenethyl-4-oxa- 7,10-diaza-1(3,1)-piperidina-5(1,3)- benzenacyclotetradecaphane-12- yl)amino)-2-oxoethyl)phenyl)carbamate |
| 443 | 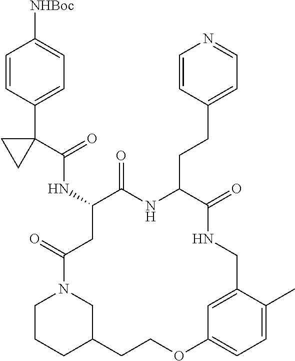 [SMILES: Cc1ccc2cc1CNC(=O)C(CCc1ccncc1)NC(=O)[C@@H](NC(=O)C1(c3ccc(NC(=O)OC(C)(C)C)cc3)CC1)CC(=O)N1CCCC(CCO2)C1] | tert-butyl (4-(1-(((12S)-5⁴-methyl-8,11,14- trioxo-9-(2-(pyridin-4-yl)ethyl)-4-oxa-7,10- diaza-1(3,1)-piperidina-5(1,3)- benzenacyclotetradecaphane-12- yl)carbamoyl)cyclopropyl)phenyl) carbamate |
| 444 | 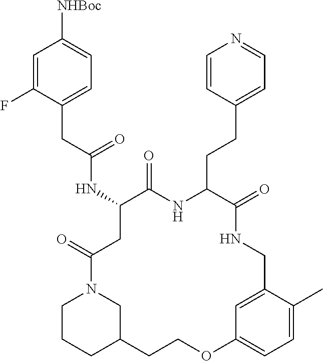 [SMILES: Cc1ccc2cc1CNC(=O)C(CCc1ccncc1)NC(=O)[C@@H](NC(=O)Cc1ccc(NC(=O)OC(C)(C)C)cc1F)CC(=O)N1CCCC(CCO2)C1] | tert-butyl (3-fluoro-4-(2-(((12S)-5⁴-methyl- 8,11,14-trioxo-9-(2-(pyridin-4-yl)ethyl)-4- oxa-7,10-diaza-1(3,1)-piperidina-5(1,3)- benzenacyclotetradecaphane-12- yl)amino)-2-oxoethyl)phenyl)carbamate |
| 479 | 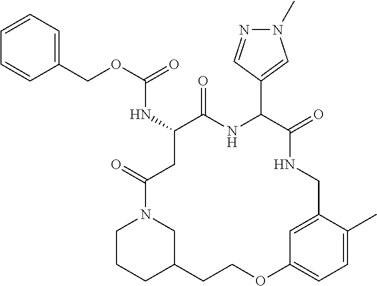 [SMILES: Cc1ccc2cc1CNC(=O)C(c1cnn(C)c1)NC(=O)[C@@H](NC(=O)OCc1ccccc1)CC(=O)N1CCCC(CCO2)C1] | Benzyl ((12S)-5⁴-methyl-9-(1-methyl-1H- pyrazol-4-yl)-8,11,14-trioxo-4-oxa-7,10- diaza-1(3,1)-piperidina-5(1,3)- benzenacyclotetradecaphane-12- yl)carbamate |
| 480 | 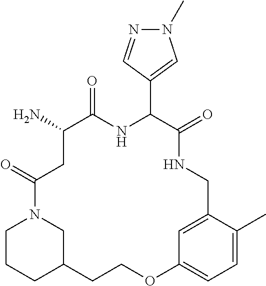 [SMILES: Cc1ccc2cc1CNC(=O)C(c1cnn(C)c1)NC(=O)[C@@H](N)CC(=O)N1CCCC(CCO2)C1] | (12S)-12-amino-5⁴-methyl-9-(1-methyl- 1H-pyrazol-4-yl)-4-oxa-7,10-diaza-1(3,1)- piperidina-5(1,3)- benzenacyclotetradecaphane-8,11,14- trione |
| 495 | 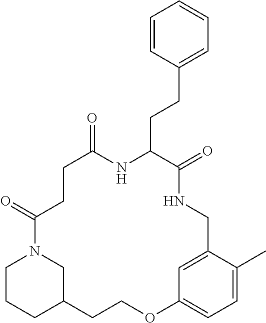 [SMILES: Cc1ccc2cc1CNC(=O)C(CCc1ccccc1)NC(=O)CCC(=O)N1CCCC(CCO2)C1] | 5⁴-methyl-9-phenethyl-4-oxa-7,10-diaza- 1(3,1)-piperidina-5(1,3)- benzenacyclotetradecaphane-8,11,14- trione |
| 503 | 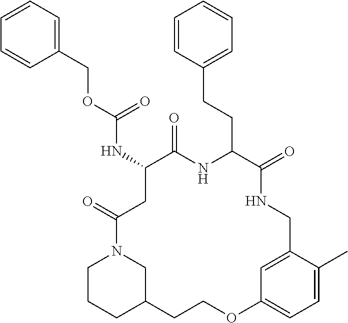 [SMILES: Cc1ccc2cc1CNC(=O)C(CCc1ccccc1)NC(=O)[C@@H](NC(=O)OCc1ccccc1)CC(=O)N1CCCC(CCO2)C1] | benzyl ((12S)-9-phenethyl-5⁴-methyl- 8,11,14-trioxo-4-oxa-7,10-diaza-1(3,1)- piperidina-5(1,3)- benzenacyclotetradecaphane-12- yl)carbamate |
| 505 | 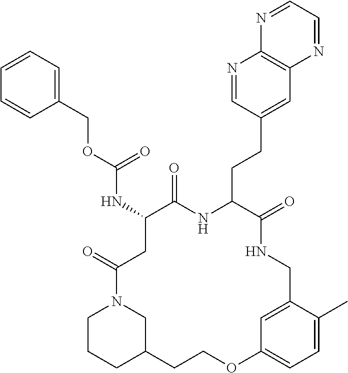 [SMILES: Cc1ccc2cc1CNC(=O)C(CCc1cnc3nccnc3c1)NC(=O)[C@@H](NC(=O)OCc1ccccc1)CC(=O)N1CCCC(CCO2)C1] | benzyl ((12S)-5⁴-methyl-8,11,14-trioxo-9- (2-(pyrido[2,3-b]pyrazin-7-yl)ethyl)-4-oxa- 7,10-diaza-1(3,1)-piperidina-5(1,3)- benzenacyclotetradecaphane-12- yl)carbamate |
| 509 | 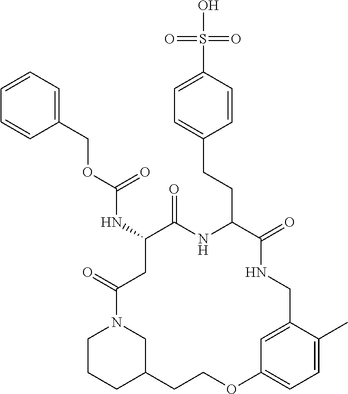 [SMILES: Cc1ccc2cc1CNC(=O)C(CCc1ccc(S(=O)(=O)O)cc1)NC(=O)[C@@H](NC(=O)OCc1ccccc1)CC(=O)N1CCCC(CCO2)C1] | 4-(2-((12S)-12- (((benzyloxy)carbonyl)amino)-5⁴-methyl- 8,11,14-trioxo-4-oxa-7,10-diaza-1(3,1)- piperidina-5(1,3)- benzenacyclotetradecaphane-9- yl)ethyl)benzenesulfonic acid |
| 510 | 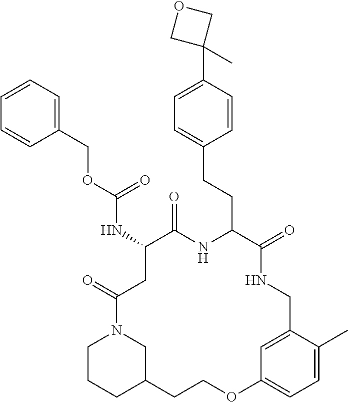 [SMILES: Cc1ccc2cc1CNC(=O)C(CCc1ccc(C3(C)COC3)cc1)NC(=O)[C@@H](NC(=O)OCc1ccccc1)CC(=O)N1CCCC(CCO2)C1] | benzyl ((12S)-5⁴-methyl-9-(4-(3- methyloxetan-3-yl)phenethyl)-8,11,14- trioxo-4-oxa-7,10-diaza-1(3,1)-piperidina- 5(1,3)-benzenacyclotetradecaphane-12- yl)carbamate |
| 511 | 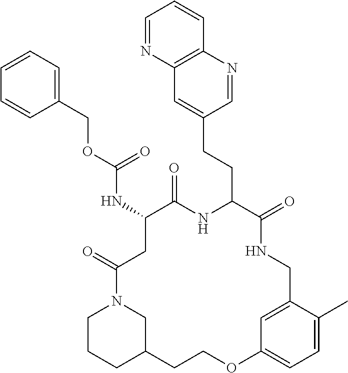 [SMILES: Cc1ccc2cc1CNC(=O)C(CCc1cnc3cccnc3c1)NC(=O)[C@@H](NC(=O)OCc1ccccc1)CC(=O)N1CCCC(CCO2)C1] | benzyl ((12S)-9-(2-(1,5-naphthyridin-3- yl)ethyl)-5⁴-methyl-8,11,14-trioxo-4-oxa- 7,10-diaza-1(3,1)-piperidina-5(1,3)- benzenacyclotetradecaphane-12- yl)carbamate |
| 512 | 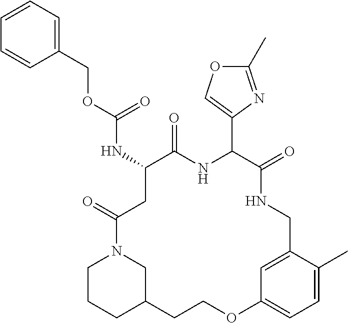 [SMILES: Cc1nc(C2NC(=O)[C@@H](NC(=O)OCc3ccccc3)CC(=O)N3CCCC(CCOc4ccc(C)c(c4)CNC2=O)C3)co1] | benzyl ((12S)-5⁴-methyl-9-(2- methyloxazol-4-yl)-8,11,14-trioxo-4-oxa- 7,10-diaza-1(3,1)-piperidina-5(1,3)- benzenacyclotetradecaphane-12- yl)carbamate |
| 513 | 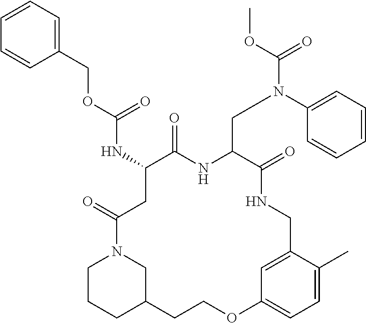 [SMILES: COC(=O)N(CC1NC(=O)[C@@H](NC(=O)OCc2ccccc2)CC(=O)N2CCCC(CCOc3ccc(C)c(c3)CNC1=O)C2)c1ccccc1] | methyl (((12R)-12- (((benzyloxy)carbonyl)amino)-5⁴-methyl- 8,11,14-trioxo-4-oxa-7,10-diaza-1(3,1)- piperidina-5(1,3)- benzenacyclotetradecaphane-9- yl)methyl)(phenyl)carbamate |
| 549 | 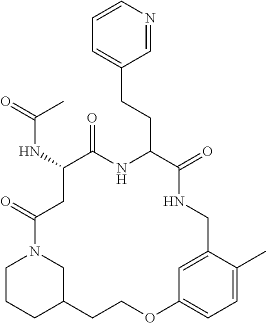 [SMILES: CC(=O)N[C@H]1CC(=O)N2CCCC(CCOc3ccc(C)c(c3)CNC(=O)C(CCc3cccnc3)NC1=O)C2] | N-((12S)-5⁴-methyl-8,11,14-trioxo-9-(2- (pyridin-3-yl)ethyl)-4-oxa-7,10-diaza- 1(3,1)-piperidina-5(1,3)- benzenacyclotetradecaphane-12- yl)acetamide |
| 550 | 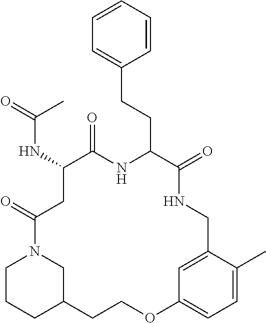 [SMILES: CC(=O)N[C@H]1CC(=O)N2CCCC(CCOc3ccc(C)c(c3)CNC(=O)C(CCc3ccccc3)NC1=O)C2] | N-((12S)-5⁴-methyl-8,11,14-trioxo-9- phenethyl-4-oxa-7,10-diaza-1(3,1)- piperidina-5(1,3)- benzenacyclotetradecaphane-12- yl)acetamide |
| 551 | 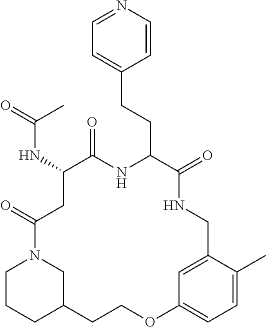 [SMILES: CC(=O)N[C@H]1CC(=O)N2CCCC(CCOc3ccc(C)c(c3)CNC(=O)C(CCc3ccncc3)NC1=O)C2] | N-((12S)-5⁴-methyl-8,11,14-trioxo-9-(2- (pyridin-4-yl)ethyl)-4-oxa-7,10-diaza- 1(3,1)-piperidina-5(1,3)- benzenacyclotetradecaphane-12- yl)acetamide |
| 552 | 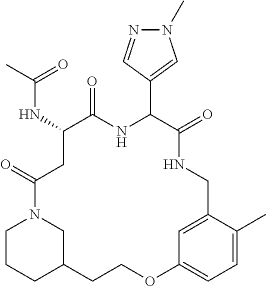 [SMILES: CC(=O)N[C@H]1CC(=O)N2CCCC(CCOc3ccc(C)c(c3)CNC(=O)C(c3cnn(C)c3)NC1=O)C2] | N-((12S)-5⁴-methyl-9-(1-methyl-1H- pyrazol-4-yl)-8,11,14-trioxo-4-oxa-7,10- diaza-1(3,1)-piperidina-5(1,3)- benzenacyclotetradecaphane-12- yl)acetamide |
| 553 | 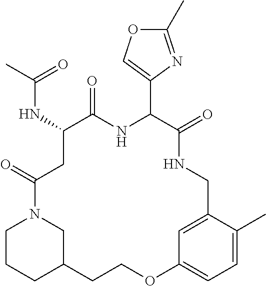 [SMILES: CC(=O)N[C@H]1CC(=O)N2CCCC(CCOc3ccc(C)c(c3)CNC(=O)C(c3coc(C)n3)NC1=O)C2] | N-((12S)-5⁴-methyl-9-(2-methyloxazol-4- yl)-8,11,14-trioxo-4-oxa-7,10-diaza- 1(3,1)-piperidina-5(1,3)- benzenacyclotetradecaphane-12- yl)acetamide |
| 554 | 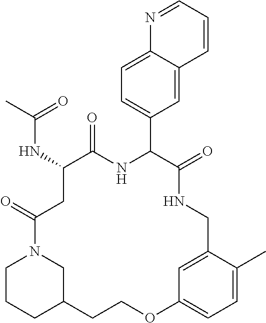 [SMILES: CC(=O)N[C@H]1CC(=O)N2CCCC(CCOc3ccc(C)c(c3)CNC(=O)C(c3ccc4ncccc4c3)NC1=O)C2] | N-((12S)-5⁴-methyl-8,11,14-trioxo-9- (quinolin-6-yl)-4-oxa-7,10-diaza-1(3,1)- piperidina-5(1,3)- benzenacyclotetradecaphane-12- yl)acetamide |
| 555 | 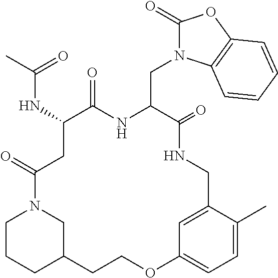 [SMILES: CC(=O)N[C@H]1CC(=O)N2CCCC(CCOc3ccc(C)c(c3)CNC(=O)C(Cn3c(=O)oc4ccccc43)NC1=O)C2] | N-((12S)-5⁴-methyl-8,11,14-trioxo-9-((2- oxobenzo[d]oxazol-3(2H)-yl)methyl)-4- oxa-7,10-diaza-1(3,1)-piperidina-5(1,3)- benzenacyclotetradecaphane-12- yl)acetamide |
| 556 | 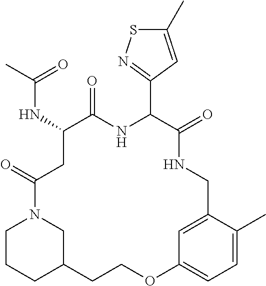 [SMILES: CC(=O)N[C@H]1CC(=O)N2CCCC(CCOc3ccc(C)c(c3)CNC(=O)C(c3cc(C)sn3)NC1=O)C2] | N-((12S)-5⁴-methyl-9-(5-methylisothiazol- 3-yl)-8,11,14-trioxo-4-oxa-7,10-diaza- 1(3,1)-piperidina-5(1,3)- benzenacyclotetradecaphane-12- yl)acetamide |
| 557 | 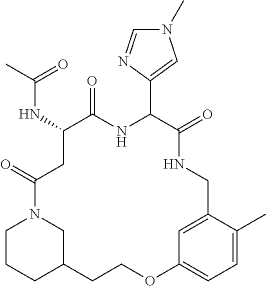 [SMILES: CC(=O)N[C@H]1CC(=O)N2CCCC(CCOc3ccc(C)c(c3)CNC(=O)C(c3cn(C)cn3)NC1=O)C2] | N-((12S)-5⁴-methyl-9-(1-methyl-1H- imidazol-4-yl)-8,11,14-trioxo-4-oxa-7,10- diaza-1(3,1)-piperidina-5(1,3)- benzenacyclotetradecaphane-12- yl)acetamide |
| 558 | 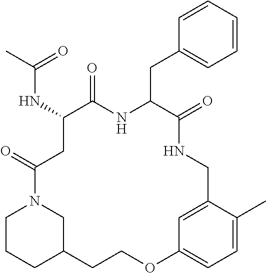 [SMILES: CC(=O)N[C@H]1CC(=O)N2CCCC(CCOc3ccc(C)c(c3)CNC(=O)C(Cc3ccccc3)NC1=O)C2] | N-((12S)-9-benzyl-5⁴-methyl-8,11,14- trioxo-4-oxa-7,10-diaza-1(3,1)-piperidina- 5(1,3)-benzenacyclotetradecaphane-12- yl)acetamide |
| 559 | 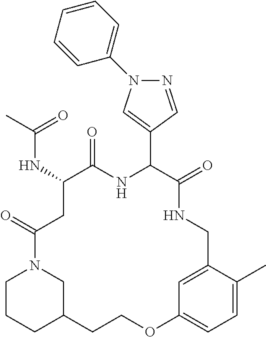 [SMILES: CC(=O)N[C@H]1CC(=O)N2CCCC(CCOc3ccc(C)c(c3)CNC(=O)C(c3cnn(-c4ccccc4)c3)NC1=O)C2] | N-((12S)-5⁴-methyl-8,11,14-trioxo-9-(1- phenyl-1H-pyrazol-4-yl)-4-oxa-7,10-diaza- 1(3,1)-piperidina-5(1,3)- benzenacyclotetradecaphane-12- yl)acetamide |
| 560 | 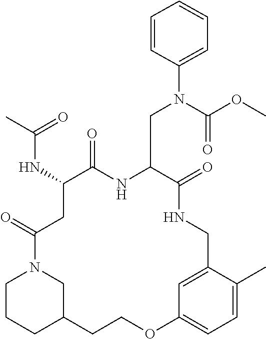 [SMILES: COC(=O)N(CC1NC(=O)[C@@H](NC(C)=O)CC(=O)N2CCCC(CCOc3ccc(C)c(c3)CNC1=O)C2)c1ccccc1] | methyl (((12S)-12-acetamido-5⁴-methyl- 8,11,14-trioxo-4-oxa-7,10-diaza-1(3,1)- piperidina-5(1,3)- benzenacyclotetradecaphane-9- yl)methyl)(phenyl)carbamate |
| 561 | 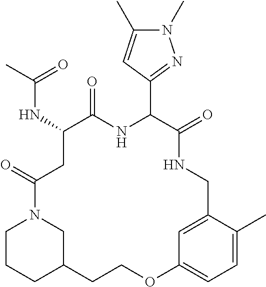 [SMILES: CC(=O)N[C@H]1CC(=O)N2CCCC(CCOc3ccc(C)c(c3)CNC(=O)C(c3cc(C)n(C)n3)NC1=O)C2] | N-((12S)-9-(1,5-dimethyl-1H-pyrazol-3- yl)-5⁴-methyl-8,11,14-trioxo-4-oxa-7,10- diaza-1(3,1)-piperidina-5(1,3)- benzenacyclotetradecaphane-12- yl)acetamide |
| 562 | 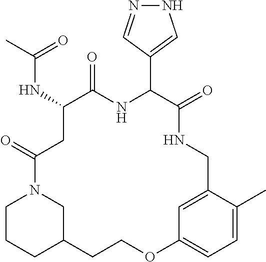 [SMILES: CC(=O)N[C@H]1CC(=O)N2CCCC(CCOc3ccc(C)c(c3)CNC(=O)C(c3cn[nH]c3)NC1=O)C2] | N-((12S)-5⁴-methyl-8,11,14-trioxo-9-(1H- pyrazol-4-yl)-4-oxa-7,10-diaza-1(3,1)- piperidina-5(1,3)- benzenacyclotetradecaphane-12- yl)acetamide |
| 563 | 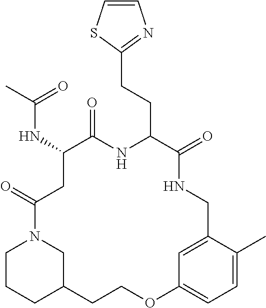 [SMILES: CC(=O)N[C@H]1CC(=O)N2CCCC(CCOc3ccc(C)c(c3)CNC(=O)C(CCc3nccs3)NC1=O)C2] | N-((12S)-5⁴-methyl-8,11,14-trioxo-9-(2- (thiazol-2-yl)ethyl)-4-oxa-7,10-diaza- 1(3,1)-piperidina-5(1,3)- benzenacyclotetradecaphane-12- yl)acetamide |
| 564 | 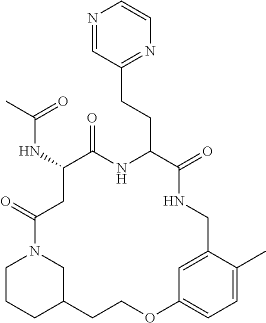 [SMILES: CC(=O)N[C@H]1CC(=O)N2CCCC(CCOc3ccc(C)c(c3)CNC(=O)C(CCc3cnccn3)NC1=O)C2] | N-((12S)-5⁴-methyl-8,11,14-trioxo-9-(2- (pyrazin-2-yl)ethyl)-4-oxa-7,10-diaza- 1(3,1)-piperidina-5(1,3)- benzenacyclotetradecaphane-12- yl)acetamide |
| 565 | 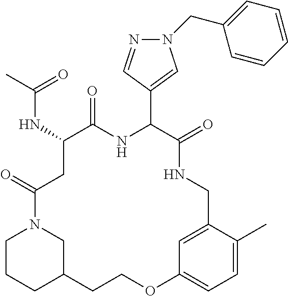 [SMILES: CC(=O)N[C@H]1CC(=O)N2CCCC(CCOc3ccc(C)c(c3)CNC(=O)C(c3cnn(Cc4ccccc4)c3)NC1=O)C2] | N-((12S)-9-(1-benzyl-1H-pyrazol-4-yl)-5⁴- methyl-8,11,14-trioxo-4-oxa-7,10-diaza- 1(3,1)-piperidina-5(1,3)- benzenacyclotetradecaphane-12- yl)acetamide |
| 566 | 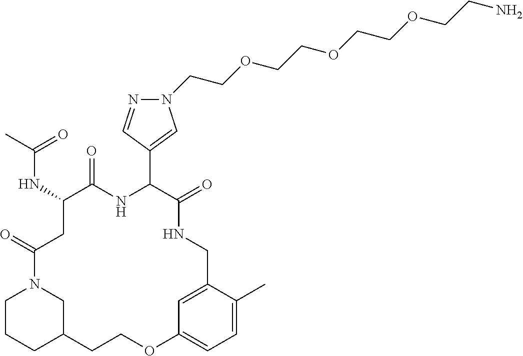 [SMILES: CC(=O)N[C@H]1CC(=O)N2CCCC(CCOc3ccc(C)c(c3)CNC(=O)C(c3cnn(CCOCCOCCOCCN)c3)NC1=O)C2] | N-((12S)-9-(1-(2-(2-(2-(2- aminoethoxy)ethoxy)ethoxy)ethyl)-1H- pyrazol-4-yl)-5⁴-methyl-8,11,14-trioxo-4- oxa-7,10-diaza-1(3,1)-piperidina-5(1,3)- benzenacyclotetradecaphane-12- yl)acetamide |
| 567 | 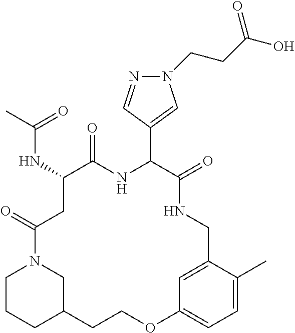 [SMILES: CC(=O)N[C@H]1CC(=O)N2CCCC(CCOc3ccc(C)c(c3)CNC(=O)C(c3cnn(CCC(=O)O)c3)NC1=O)C2] | 3-(4-((12S)-12-acetamido-5⁴-methyl- 8,11,14-trioxo-4-oxa-7,10-diaza-1(3,1)- piperidina-5(1,3)- benzenacyclotetradecaphane-9-yl)-1H- pyrazol-1-yl)propanoic acid |
| 568 | 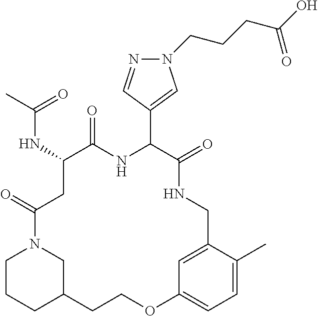 [SMILES: CC(=O)N[C@H]1CC(=O)N2CCCC(CCOc3ccc(C)c(c3)CNC(=O)C(c3cnn(CCCC(=O)O)c3)NC1=O)C2] | 4-(4-((12S)-12-acetamido-5⁴-methyl- 8,11,14-trioxo-4-oxa-7,10-diaza-1(3,1)- piperidina-5(1,3)- benzenacyclotetradecaphane-9-yl)-1H- pyrazol-1-yl)butanoic acid |
| 570 | 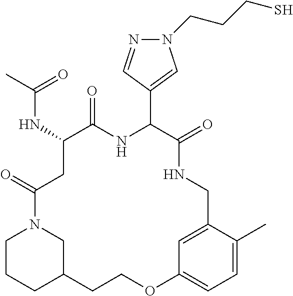 [SMILES: CC(=O)N[C@H]1CC(=O)N2CCCC(CCOc3ccc(C)c(c3)CNC(=O)C(c3cnn(CCCS)c3)NC1=O)C2] | N-((12S)-9-(1-(3-mercaptopropyl)-1H- pyrazol-4-yl)-5⁴-methyl-8,11,14-trioxo-4- oxa-7,10-diaza-1(3,1)-piperidina-5(1,3)- benzenacyclotetradecaphane-12- yl)acetamide |
| 571 | 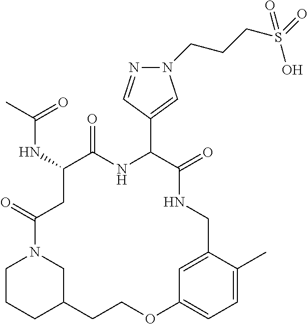 [SMILES: CC(=O)N[C@H]1CC(=O)N2CCCC(CCOc3ccc(C)c(c3)CNC(=O)C(c3cnn(CCCS(=O)(=O)O)c3)NC1=O)C2] | 3-(4-((12S)-12-acetamido-5⁴-methyl- 8,11,14-trioxo-4-oxa-7,10-diaza-1(3,1)- piperidina-5(1,3)- benzenacyclotetradecaphane-9-yl)-1H- pyrazol-1-yl)propane-1-sulfonic acid |
| 572 | 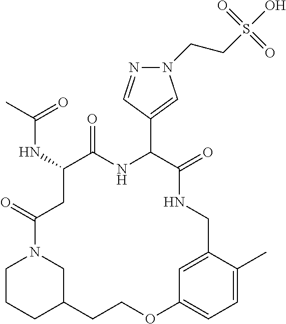 [SMILES: CC(=O)N[C@H]1CC(=O)N2CCCC(CCOc3ccc(C)c(c3)CNC(=O)C(c3cnn(CCS(=O)(=O)O)c3)NC1=O)C2] | N-(4-((12S)-12-acetamido-5⁴-methyl- 8,11,14-trioxo-4-oxa-7,10-diaza-1(3,1)- piperidina-5(1,3)- benzenacyclotetradecaphane-9-yl)-1H- pyrazol-1-yl)ethane-1-sulfonic acid |
| 573 | 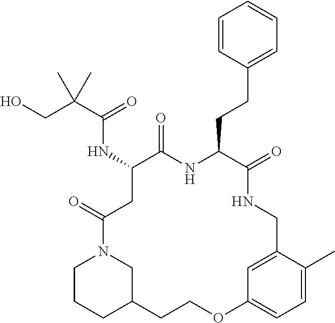 [SMILES: Cc1ccc2cc1CNC(=O)[C@H](CCc1ccccc1)NC(=O)[C@@H](NC(=O)C(C)(C)CO)CC(=O)N1CCCC(CCO2)C1] | 3-hydroxy-2,2-dimethyl-N-((13R,9S,12S)-5⁴- methyl-8,11,14-trioxo-9-phenethyl-4-oxa- 7,10-diaza-1(3,1)-piperidina-5(1,3)- benzenacyclotetradecaphane-12- yl)propanamide |
| 574 | 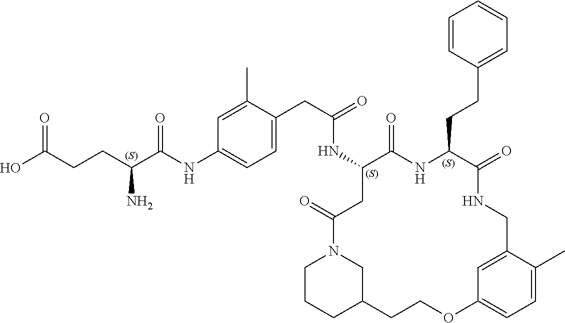 | (4S)-4-amino-5-((3-methyl-4-(2-(((9S,12S)-5⁴- methyl-8,11,14-trioxo-9-phenethyl-4-oxa- 7,10-diaza-1(3,1)-piperidina-5(1,3)- benzenacyclotetradecaphane-12-yl)amino)-2- oxoethyl)phenyl)amino)-5-oxopentanoic acid |
| 575 | 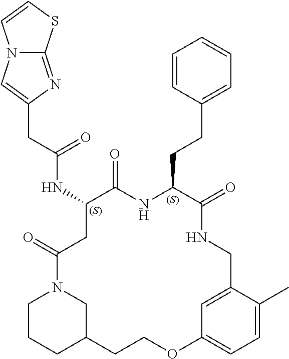 | N-((9S,12S)-9-(2-cyclohexylethyl)-5⁴-methyl- 8,11,14-trioxo-4-oxa-7,10-diaza-1(3,1)- piperidina-5(1,3)- benzenacyclotetradecaphane-12-yl)-2- (imidazo[2,1-b]thiazol-6-yl)acetamide |
| 576 | 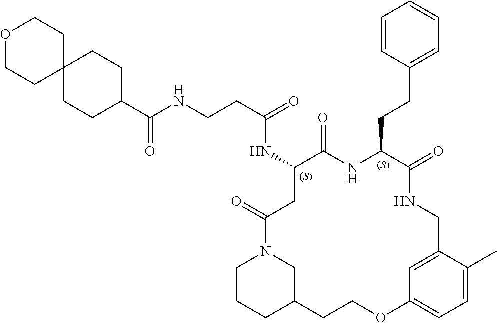 | N-(3-(((9S,12S)-5⁴-methyl-8,11,14-trioxo-9- phenethyl-4-oxa-7,10-diaza-1(3,1)-piperidina- 5(1,3)-benzenacyclotetradecaphane-12- yl)amino)-3-oxopropyl)-3- oxaspiro[5.5]undecane-9-carboxamide |
| 577 | 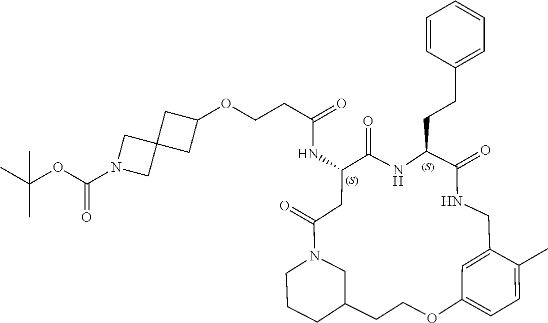 | tert-butyl 6-(3-(((9S,12S)-5⁴-methyl-8,11,14- trioxo-9-phenethyl-4-oxa-7,10-diaza-1(3,1)- piperidina-5(1,3)- benzenacyclotetradecaphane-12-yl)amino)-3- oxopropoxy)-2-azaspiro[3.3]heptane-2- carboxylate |
| 578 | 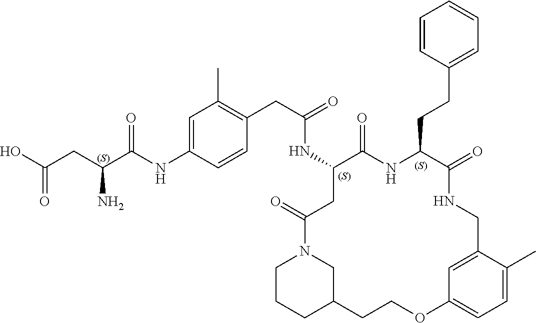 | (3S)-3-amino-4-((3-methyl-4-(2-(((9S,12S)-5⁴- methyl-8,11,14-trioxo-9-phenethyl-4-oxa- 7,10-diaza-1(3,1)-piperidina-5(1,3)- benzenacyclotetradecaphane-12-yl)amino)-2- oxoethyl)phenyl)amino)-4-oxobutanoic acid |
| 579 | 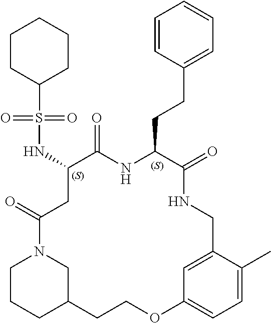 | N-((9S,12S)-5⁴-methyl-8,11,14-trioxo-9- phenethyl-4-oxa-7,10-diaza-1(3,1)-piperidina- 5(1,3)-benzenacyclotetradecaphane-12- yl)cyclohexanesulfonamide |
| 580 | 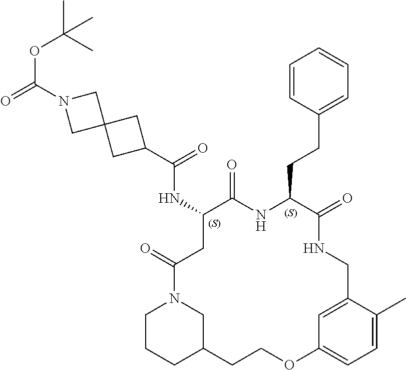 | tert-butyl 6-(((9S,12S)-5⁴-methyl-8,11,14- trioxo-9-phenethyl-4-oxa-7,10-diaza-1(3,1)- piperidina-5(1,3)- benzenacyclotetradecaphane-12- yl)carbamoyl)-2-azaspiro[3.3]heptane-2- carboxylate |
| 581 | 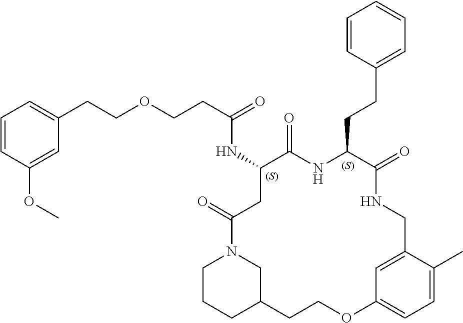 | 3-(3-methoxyphenethoxy)-N-((9S,12S)-5⁴- methyl-8,11,14-trioxo-9-phenethyl-4-oxa- 7,10-diaza-1(3,1)-piperidina-5(1,3)- benzenacyclotetradecaphane-12- yl)propanamide |
| 582 | 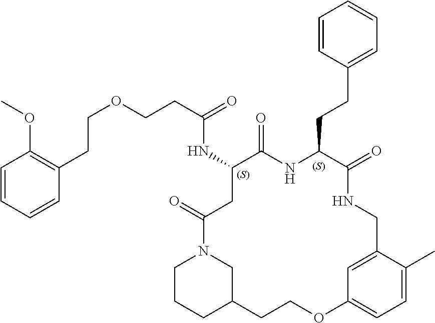 | 3-(2-methoxyphenethoxy)-N-((9S,12S)-5⁴- methyl-8,11,14-trioxo-9-phenethyl-4-oxa- 7,10-diaza-1(3,1)-piperidina-5(1,3)- benzenacyclotetradecaphane-12- yl)propanamide |
| 583 | 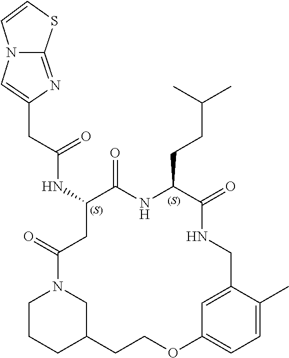 | 2-(imidazo[2,1-b]thiazol-6-yl)-N-((9S,12S)-9- isopentyl-5⁴-methyl-8,11,14-trioxo-4-oxa- 7,10-diaza-1(3,1)-piperidina-5(1,3)- benzenacyclotetradecaphane-12- yl)acetamide |
| 584 | 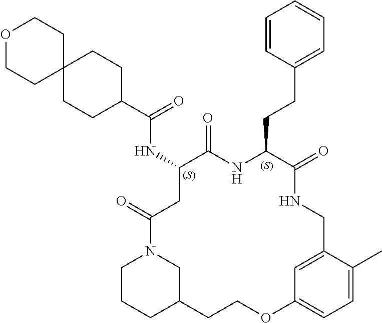 | N-((9S,12S)-5⁴-methyl-8,11,14-trioxo-9- phenethyl-4-oxa-7,10-diaza-1(3,1)-piperidina- 5(1,3)-benzenacyclotetradecaphane-12-yl)-3- oxaspiro[5.5]undecane-9-carboxamide |
| 585 | 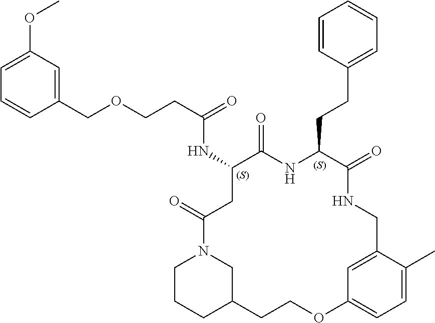 | 3-((3-methoxybenzyl)oxy)-N-((9S,12S)-5⁴- methyl-8,11,14-trioxo-9-phenethyl-4-oxa- 7,10-diaza-1(3,1)-piperidina-5(1,3)- benzenacyclotetradecaphane-12- yl)propanamide |
| 586 | 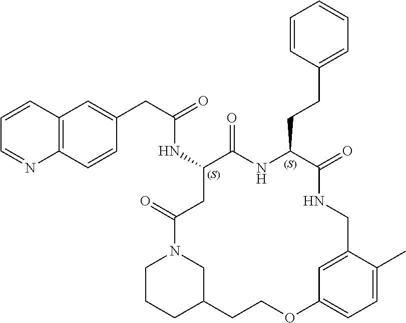 | N-((9S,12S)-5⁴-methyl-8,11,14-trioxo-9- phenethyl-4-oxa-7,10-diaza-1(3,1)-piperidina- 5(1,3)-benzenacyclotetradecaphane-12-yl)-2- (quinolin-6-yl)acetamide |
| 587 | 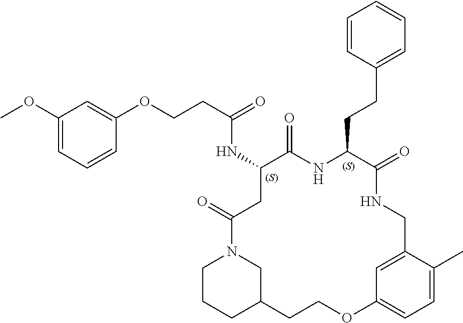 | 3-(3-methoxyphenoxy)-N-((9S,12S)-5⁴- methyl-8,11,14-trioxo-9-phenethyl-4-oxa- 7,10-diaza-1(3,1)-piperidina-5(1,3)- benzenacyclotetradecaphane-12- yl)propanamid |
| 588 | 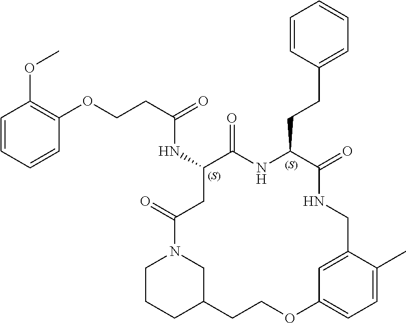 | 3-(2-methoxyphenoxy)-N-((9S,12S)-5⁴- methyl-8,11,14-trioxo-9-phenethyl-4-oxa- 7,10-diaza-1(3,1)-piperidina-5(1,3)- benzenacyclotetradecaphane-12- yl)propanamide |
| 589 | 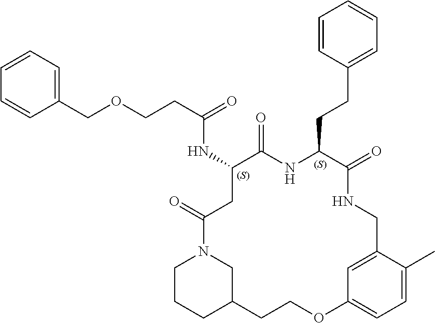 | 3-(benzyloxy)-N-((9S,12S)-5⁴-methyl-8,11,14- trioxo-9-phenethyl-4-oxa-7,10-diaza-1(3,1)- piperidina-5(1,3)- benzenacyclotetradecaphane-12- yl)propanamide |
| 590 | 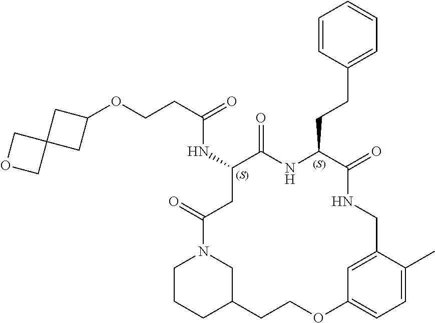 | 3-((2-oxaspiro[3.3]heptan-6-yl)oxy)-N- ((9S,12S)-5⁴-methyl-8,11,14-trioxo-9- phenethyl-4-oxa-7,10-diaza-1(3,1)-piperidina- 5(1,3)-benzenacyclotetradecaphane-12- yl)propanamide |
| 591 | 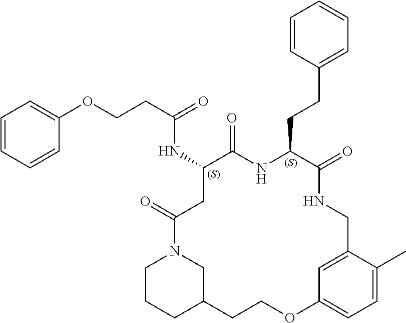 | N-((9S,12S)-5⁴-methyl-8,11,14-trioxo-9- phenethyl-4-oxa-7,10-diaza-1(3,1)-piperidina- 5(1,3)-benzenacyclotetradecaphane-12-yl)-3- phenoxypropanamide |
| 592 | 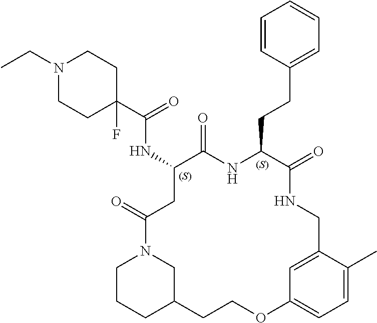 | 1-ethyl-4-fluoro-N-((9S,12S)-5⁴-methyl- 8,11,14-trioxo-9-phenethyl-4-oxa-7,10-diaza- 1(3,1)-piperidina-5(1,3)- benzenacyclotetradecaphane-12- yl)piperidine-4-carboxamide |
| 593 | 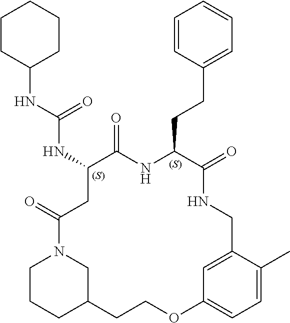 | 1-cyclohexyl-3-((9S,12S)-5⁴-methyl-8,11,14- trioxo-9-phenethyl-4-oxa-7,10-diaza-1(3,1)- piperidina-5(1,3)- benzenacyclotetradecaphane-12-yl)urea |
| 594 | 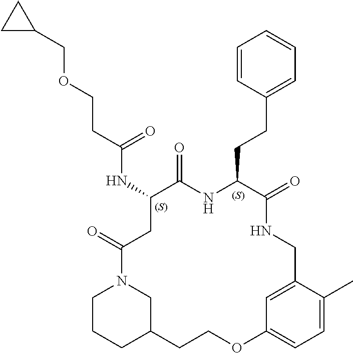 | 3-(cyclopropylmethoxy)-N-((9S,12S)-5⁴- methyl-8,11,14-trioxo-9-phenethyl-4-oxa- 7,10-diaza-1(3,1)-piperidina-5(1,3)- benzenacyclotetradecaphane-12- yl)propanamide |
| 595 | 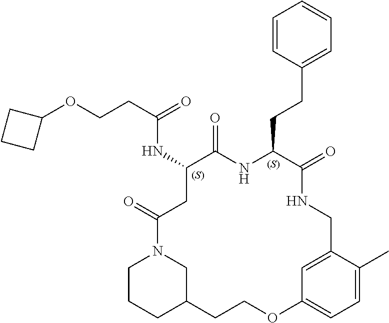 | 3-cyclobutoxy-N-((9S,12S)-5⁴-methyl- 8,11,14-trioxo-9-phenethyl-4-oxa-7,10-diaza- 1(3,1)-piperidina-5(1,3)- benzenacyclotetradecaphane-12- yl)propanamide |
| 596 | 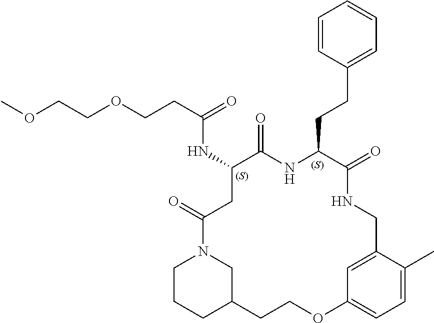 | 3-(2-methoxyethoxy)-N-((9S,12S)-5⁴-methyl- 8,11,14-trioxo-9-phenethyl-4-oxa-7,10-diaza- 1(3,1)-piperidina-5(1,3)- benzenacyclotetradecaphane-12- yl)propanamide |
| 597 | 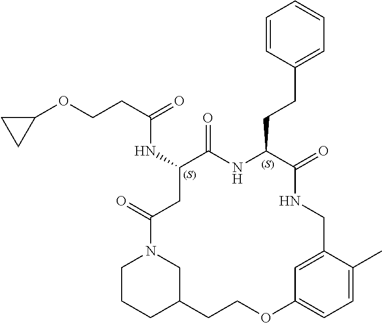 | 3-cyclopropoxy-N-((9S,12S)-5⁴-methyl- 8,11,14-trioxo-9-phenethyl-4-oxa-7,10-diaza- 1(3,1)-piperidina-5(1,3)- benzenacyclotetradecaphane-12- yl)propanamide |
| 598 | 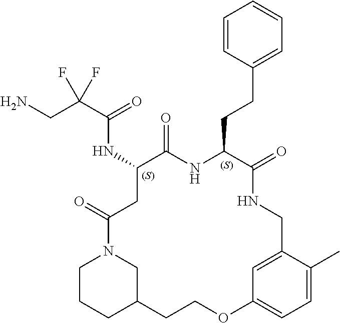 | 3-amino-2,2-difluoro-N-((9S,12S)-5⁴-methyl- 8,11,14-trioxo-9-phenethyl-4-oxa-7,10-diaza- 1(3,1)-piperidina-5(1,3)- benzenacyclotetradecaphane-12- yl)propanamide |
| 599 | 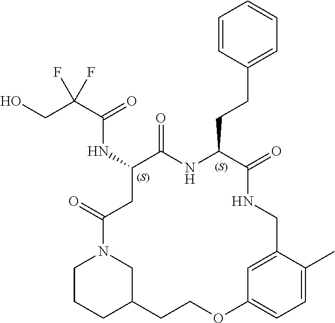 | 2,2-difluoro-3-hydroxy-N-((9S,12S)-5⁴-methyl- 8,11,14-trioxo-9-phenethyl-4-oxa-7,10-diaza- 1(3,1)-piperidina-5(1,3)- benzenacyclotetradecaphane-12- yl)propanamide |
| 600 | 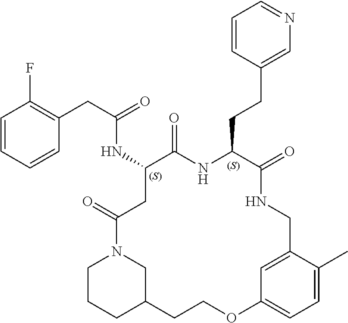 | 2-(2-fluorophenyl)-N-((9S,12S)-5⁴-methyl- 8,11,14-trioxo-9-(2-(pyridin-3-yl)ethyl)-4-oxa- 7,10-diaza-1(3,1)-piperidina-5(1,3)- benzenacyclotetradecaphane-12- yl)acetamide |
| 601 | 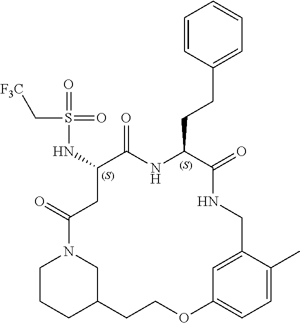 | 2,2,2-trifluoro-N-((9S,12S)-5⁴-methyl-8,11,14- trioxo-9-phenethyl-4-oxa-7,10-diaza-1(3,1)- piperidina-5(1,3)- benzenacyclotetradecaphane-12-yl)ethane-1- sulfonamide |
| 602 | 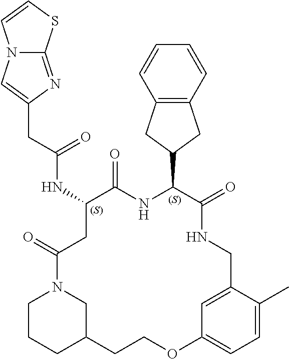 | N-((9S,12S)-9-(2,3-dihydro-1H-inden-2-yl)-5⁴- methyl-8,11,14-trioxo-4-oxa-7,10-diaza- 1(3,1)-piperidina-5(1,3)- benzenacyclotetradecaphane-12-yl)-2- (imidazo[2,1-b]thiazol-6-yl)acetamide |
| 603 | 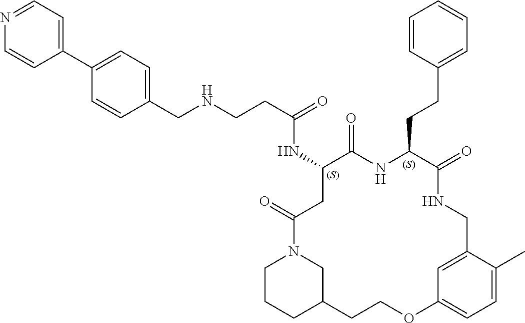 | N-((9S,12S)-5⁴-methyl-8,11,14-trioxo-9- phenethyl-4-oxa-7,10-diaza-1(3,1)-piperidina- 5(1,3)-benzenacyclotetradecaphane-12-yl)-3- ((4-(pyridin-4-yl)benzyl)amino)propanamide |
| 604 | 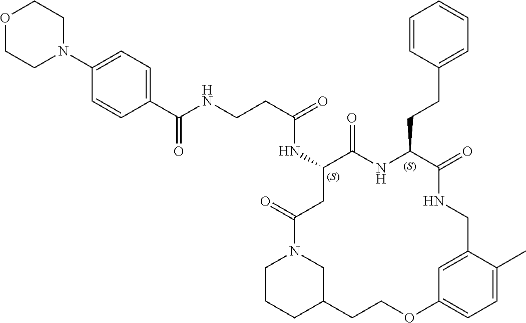 | N-(3-(((9S,12S)-5⁴-methyl-8,11,14-trioxo-9- phenethyl-4-oxa-7,10-diaza-1(3,1)-piperidina- 5(1,3)-benzenacyclotetradecaphane-12- yl)amino)-3-oxopropyl)-4- morpholinobenzamide |
| 605 | 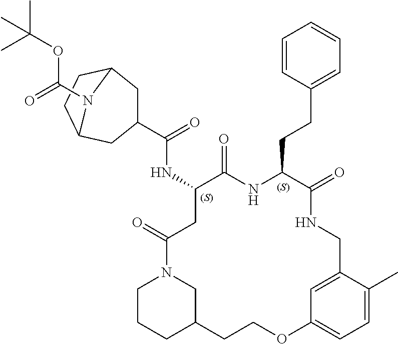 | tert-butyl 3-(((9S,12S)-5⁴-methyl-8,11,14- trioxo-9-phenethyl-4-oxa-7,10-diaza-1(3,1)- piperidina-5(1,3)- benzenacyclotetradecaphane-12- yl)carbamoyl)-8-azabicyclo[3.2.1]octane-8- carboxylate |
| 606 | 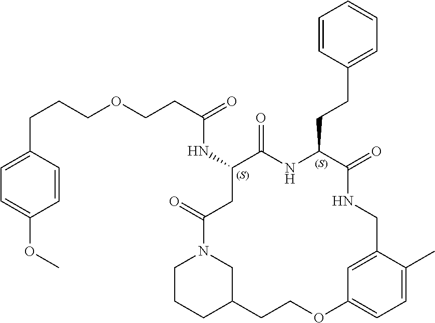 | 3-(3-(4-methoxyphenyl)propoxy)-N- ((9S,12S)-5⁴-methyl-8,11,14-trioxo-9- phenethyl-4-oxa-7,10-diaza-1(3,1)-piperidina- 5(1,3)-benzenacyclotetradecaphane-12- yl)propanamide |
| 607 | 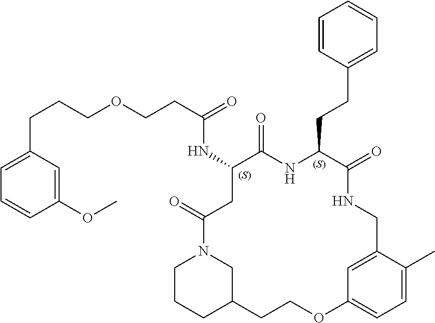 | 3-(3-(3-methoxyphenyl)propoxy)-N- ((9S,12S)-5⁴-methyl-8,11,14-trioxo-9- phenethyl-4-oxa-7,10-diaza-1(3,1)-piperidina- 5(1,3)-benzenacyclotetradecaphane-12- yl)propanamide |
| 608 | 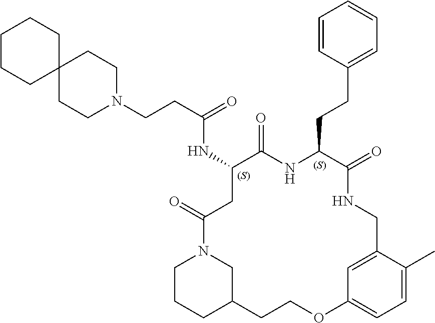 | N-((9S,12S)-5⁴-methyl-8,11,14-trioxo-9- phenethyl-4-oxa-7,10-diaza-1(3,1)-piperidina- 5(1,3)-benzenacyclotetradecaphane-12-yl)-3- (3-azaspiro[5.5]undecan-3-yl)propanamide |
| 609 | 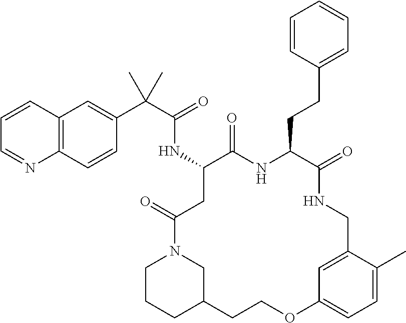 [SMILES: Cc1ccc2cc1CNC(=O)[C@H](CCc1ccccc1)NC(=O)[C@@H](NC(=O)C(C)(C)c1ccc3ncccc3c1)CC(=O)N1CCCC(CCO2)C1] | 2-methyl-N-((9S,12S)-5⁴-methyl-8,11,14- trioxo-9-phenethyl-4-oxa-7,10-diaza-1(3,1)- piperidina-5(1,3)- benzenacyclotetradecaphane-12-yl)-2- (quinolin-6-yl)propanamide |
| 610 | 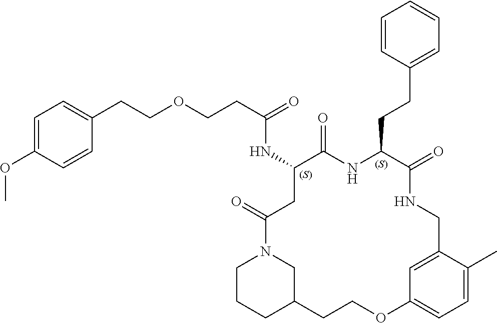 | 3-(4-methoxyphenethoxy)-N-((9S,12S)-5⁴- methyl-8,11,14-trioxo-9-phenethyl-4-oxa- 7,10-diaza-1(3,1)-piperidina-5(1,3)- benzenacyclotetradecaphane-12- yl)propanamide |
| 611 | 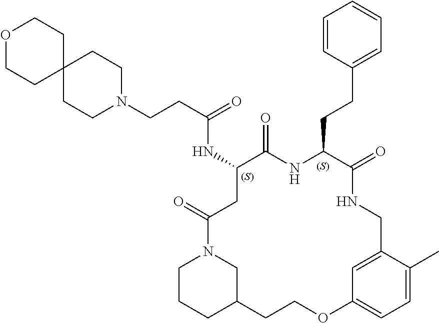 | N-((9S,12S)-5⁴-methyl-8,11,14-trioxo-9- phenethyl-4-oxa-7,10-diaza-1(3,1)-piperidina- 5(1,3)-benzenacyclotetradecaphane-12-yl)-3- (3-oxa-9-azaspiro[5.5]undecan-9- yl)propanamide |
| 612 | 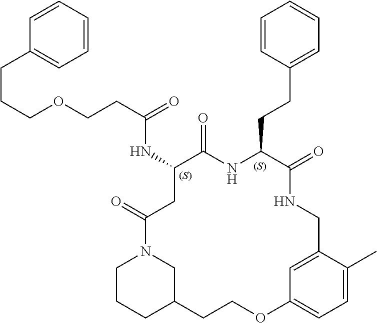 | N-((9S,12S)-5⁴-methyl-8,11,14-trioxo-9- phenethyl-4-oxa-7,10-diaza-1(3,1)-piperidina- 5(1,3)-benzenacyclotetradecaphane-12-yl)-3- (3-phenylpropoxy)propanamide |
| 613 | 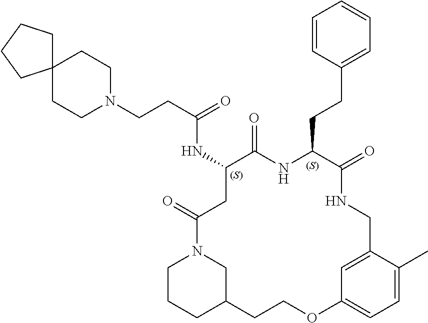 | N-((9S,12S)-5⁴-methyl-8,11,14-trioxo-9- phenethyl-4-oxa-7,10-diaza-1(3,1)-piperidina- 5(1,3)-benzenacyclotetradecaphane-12-yl)-3- (8-azaspiro[4.5]decan-8-yl)propanamide |
| 614 | 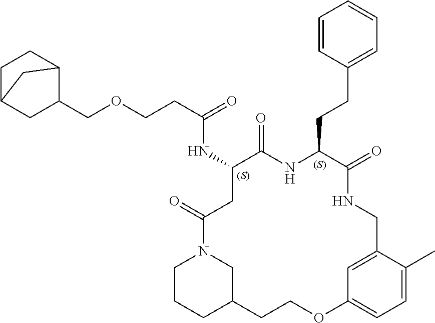 | 3-(bicyclo[2.2.1]heptan-2-ylmethoxy)-N- ((9S,12S)-5⁴-methyl-8,11,14-trioxo-9- phenethyl-4-oxa-7,10-diaza-1(3,1)-piperidina- 5(1,3)-benzenacyclotetradecaphane-12- yl)propanamide |
| 615 | 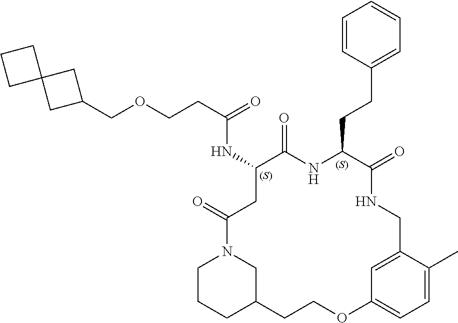 | N-((9S,12S)-5⁴-methyl-8,11,14-trioxo-9- phenethyl-4-oxa-7,10-diaza-1(3,1)-piperidina- 5(1,3)-benzenacyclotetradecaphane-12-yl)-3- (spiro[3.3]heptan-2-ylmethoxy)propanamide |
| 616 | 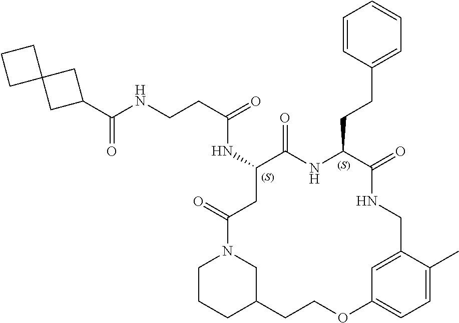 | N-(3-(((9S,12S)-5⁴-methyl-8,11,14-trioxo-9- phenethyl-4-oxa-7,10-diaza-1(3,1)-piperidina- 5(1,3)-benzenacyclotetradecaphane-12- yl)amino)-3-oxopropyl)spiro[3.3]heptane-2- carboxamide |
| 617 | 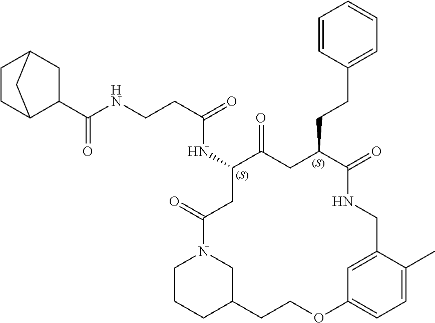 | N-(3-(((9S,12S)-5⁴-methyl-8,11,14-trioxo-9- phenethyl-4-oxa-7,10-diaza-1(3,1)-piperidina- 5(1,3)-benzenacyclotetradecaphane-12- yl)amino)-3-oxopropyl)bicyclo[2.2.1]heptane- 2-carboxamide |
| 618 | 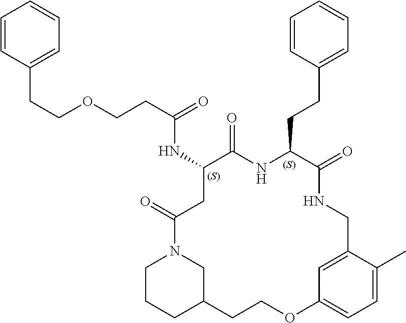 | N-((9S,12S)-5⁴-methyl-8,11,14-trioxo-9- phenethyl-4-oxa-7,10-diaza-1(3,1)-piperidina- 5(1,3)-benzenacyclotetradecaphane-12-yl)-3- phenethoxypropanamide |
| 619 | 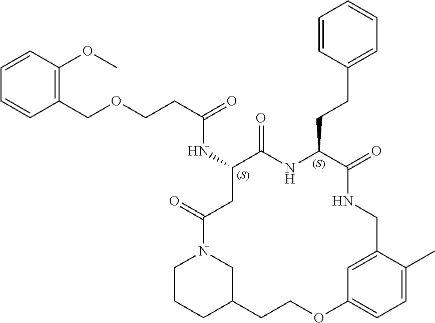 | 3-((2-methoxybenzyl)oxy)-N-((9S,12S)-5⁴- methyl-8,11,14-trioxo-9-phenethyl-4-oxa- 7,10-diaza-1(3,1)-piperidina-5(1,3)- benzenacyclotetradecaphane-12- yl)propanamide |
| 620 | 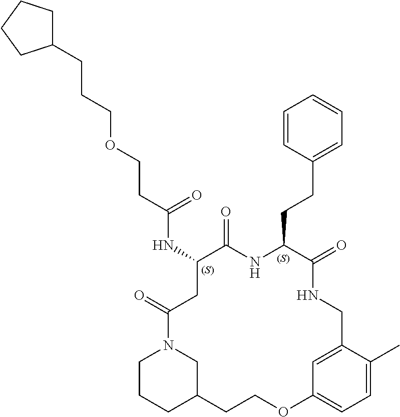 | 3-(3-cyclopentylpropoxy)-N-((9S,12S)-5⁴- methyl-8,11,14-trioxo-9-phenethyl-4-oxa- 7,10-diaza-1(3,1)-piperidina-5(1,3)- benzenacyclotetradecaphane-12- yl)propanamide |
| 621 | 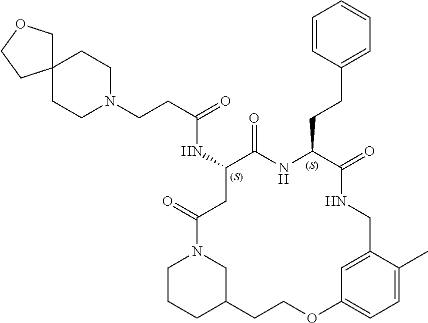 | N-((9S,12S)-5⁴-methyl-8,11,14-trioxo-9- phenethyl-4-oxa-7,10-diaza-1(3,1)-piperidina- 5(1,3)-benzenacyclotetradecaphane-12-yl)-3- (2-oxa-8-azaspiro[4.5]decan-8- yl)propanamide |
| 622 | 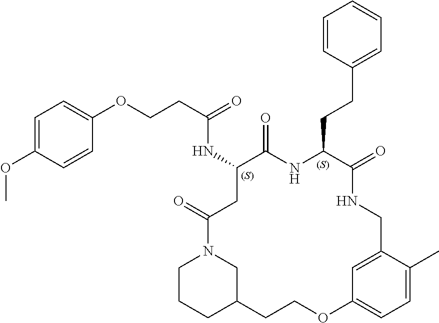 | 3-(4-methoxyphenoxy)-N-((9S,12S)-5⁴- methyl-8,11,14-trioxo-9-phenethyl-4-oxa- 7,10-diaza-1(3,1)-piperidina-5(1,3)- benzenacyclotetradecaphane-12- yl)propanamide |
| 623 | 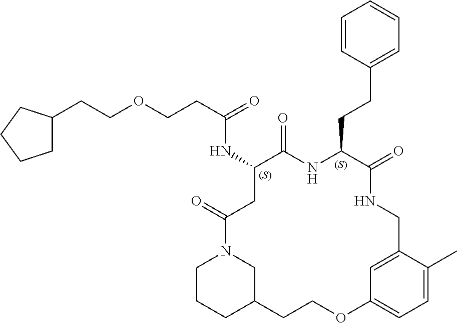 | 3-(2-cyclopentylethoxy)-N-((9S,12S)-5⁴- methyl-8,11,14-trioxo-9-phenethyl-4-oxa- 7,10-diaza-1(3,1)-piperidina-5(1,3)- benzenacyclotetradecaphane-12- yl)propanamide |
| 624 | 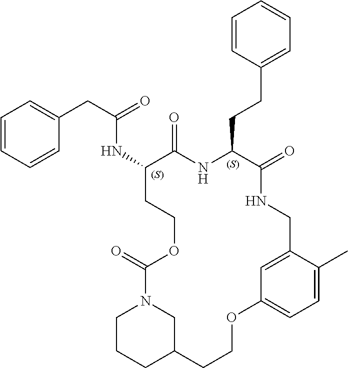 | N-((9S,12S)-5⁴-methyl-8,11,16-trioxo-9- phenethyl-4,15-dioxa-7,10-diaza-1(3,1)- piperidina-5(1,3)- benzenacyclohexadecaphane-12-yl)-2- phenylacetamide |
| 625 | 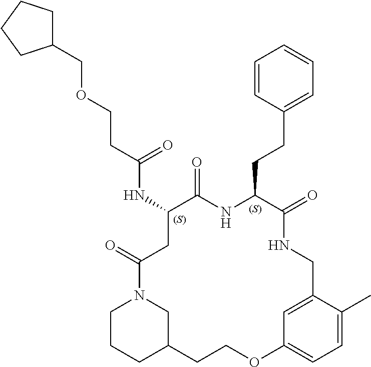 | 3-(cyclopentylmethoxy)-N-((9S,12S)-5⁴- methyl-8,11,14-trioxo-9-phenethyl-4-oxa- 7,10-diaza-1(3,1)-piperidina-5(1,3)- benzenacyclotetradecaphane-12- yl)propanamide |
| 626 | 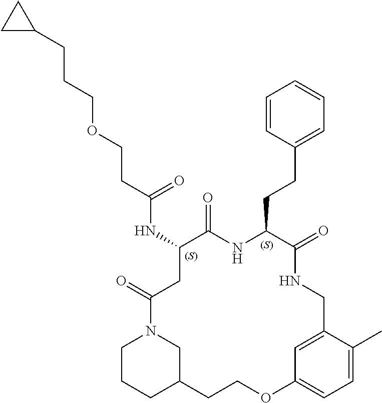 | 3-(3-cyclopropylpropoxy)-N-((9S,12S)-5⁴- methyl-8,11,14-trioxo-9-phenethyl-4-oxa- 7,10-diaza-1(3,1)-piperidina-5(1,3)- benzenacyclotetradecaphane-12- yl)propanamide |
| 627 | 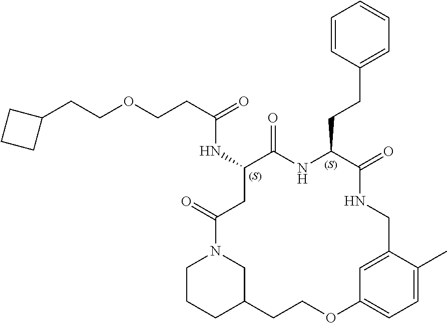 | 3-(2-cyclobutylethoxy)-N-((9S,12S)-5⁴- methyl-8,11,14-trioxo-9-phenethyl-4-oxa- 7,10-diaza-1(3,1)-piperidina-5(1,3)- benzenacyclotetradecaphane-12- yl)propanamide |
| 628 | 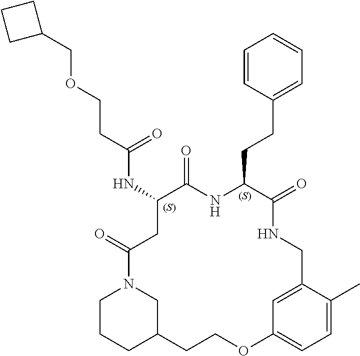 | 3-(cyclobutylmethoxy)-N-((9S,12S)-5⁴-methyl- 8,11,14-trioxo-9-phenethyl-4-oxa-7,10-diaza- 1(3,1)-piperidina-5(1,3)- benzenacyclotetradecaphane-12- yl)propanamide |
| 629 | 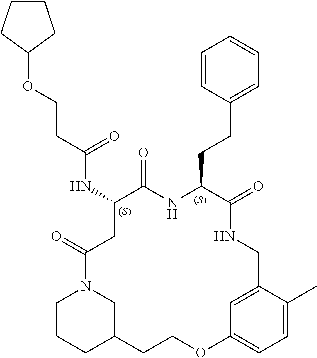 | 3-(cyclopentyloxy)-N-((9S,12S)-5⁴-methyl- 8,11,14-trioxo-9-phenethyl-4-oxa-7,10-diaza- 1(3,1)-piperidina-5(1,3)- benzenacyclotetradecaphane-12- yl)propanamide |
| 630 | 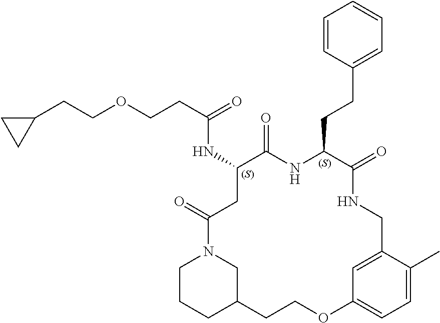 | 3-(2-cyclopropylethoxy)-N-((9S,12S)-5⁴- methyl-8,11,14-trioxo-9-phenethyl-4-oxa- 7,10-diaza-1(3,1)-piperidina-5(1,3)- benzenacyclotetradecaphane-12- yl)propanamide |
| 631 | 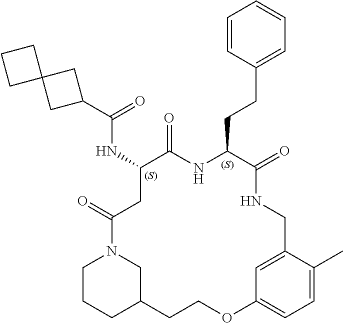 | N-((9S,12S)-5⁴-methyl-8,11,14-trioxo-9- phenethyl-4-oxa-7,10-diaza-1(3,1)-piperidina- 5(1,3)-benzenacyclotetradecaphane-12- yl)spiro[3.3]heptane-2-carboxamide |
| 632 | 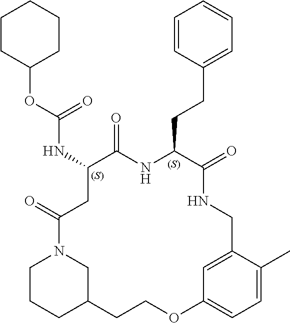 | cyclohexyl ((9S,12S)-5⁴-methyl-8,11,14- trioxo-9-phenethyl-4-oxa-7,10-diaza-1(3,1)- piperidina-5(1,3)- benzenacyclotetradecaphane-12- yl)carbamate |
| 633 | 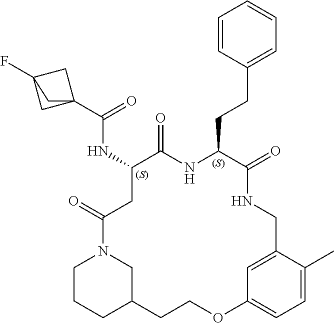 | 3-fluoro-N-((9S,12S)-5⁴-methyl-8,11,14- trioxo-9-phenethyl-4-oxa-7,10-diaza-1(3,1)- piperidina-5(1,3)- benzenacyclotetradecaphane-12- yl)bicyclo[1.1.1]pentane-1-carboxamide |
| 634 | 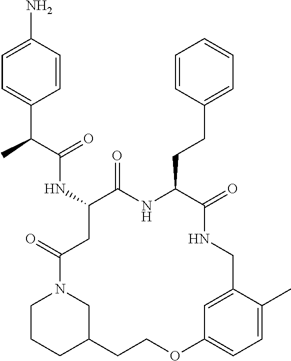 [SMILES: Cc1ccc2cc1CNC(=O)[C@H](CCc1ccccc1)NC(=O)[C@@H](NC(=O)[C@@H](C)c1ccc(N)cc1)CC(=O)N1CCCC(CCO2)C1] | (2S)-2-(4-aminophenyl)-N-((9S,12S)-5⁴- methyl-8,11,14-trioxo-9-phenethyl-4-oxa- 7,10-diaza-1(3,1)-piperidina-5(1,3)- benzenacyclotetradecaphane-12- yl)propanamide |
| 635 | 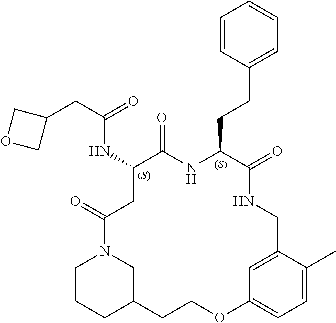 | N-((9S,12S)-5⁴-methyl-8,11,14-trioxo-9- phenethyl-4-oxa-7,10-diaza-1(3,1)-piperidina- 5(1,3)-benzenacyclotetradecaphane-12-yl)-2- (oxetan-3-yl)acetamide |
| 636 | 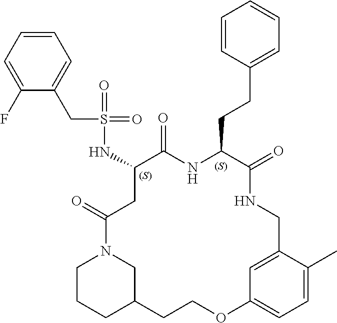 | 1-(2-fluorophenyl)-N-((9S,12S)-5⁴-methyl- 8,11,14-trioxo-9-phenethyl-4-oxa-7,10-diaza- 1(3,1)-piperidina-5(1,3)- benzenacyclotetradecaphane-12- yl)methanesulfonamide |
| 637 | 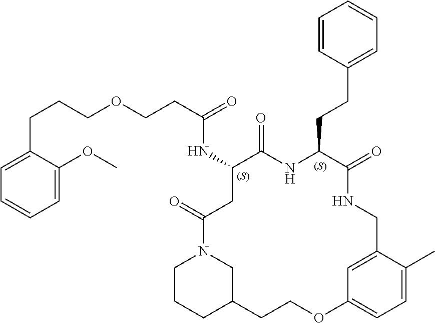 | 3-(3-(2-methoxyphenyl)propoxy)-N- ((9S,12S)-5⁴-methyl-8,11,14-trioxo-9- phenethyl-4-oxa-7,10-diaza-1(3,1)-piperidina- 5(1,3)-benzenacyclotetradecaphane-12- yl)propanamide |
| 638 | 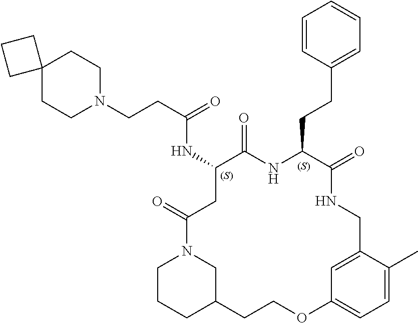 | N-((9S,12S)-5⁴-methyl-8,11,14-trioxo-9- phenethyl-4-oxa-7,10-diaza-1(3,1)-piperidina- 5(1,3)-benzenacyclotetradecaphane-12-yl)-3- (7-azaspiro[3.5]nonan-7-yl)propanamide |
| 639 | 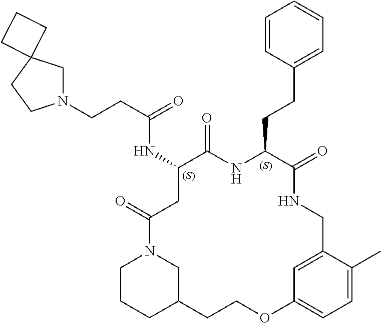 | N-((9S,12S)-5⁴-methyl-8,11,14-trioxo-9- phenethyl-4-oxa-7,10-diaza-1(3,1)-piperidina- 5(1,3)-benzenacyclotetradecaphane-12-yl)-3- (6-azaspiro[3.4]octan-6-yl)propanamide |
| 640 | 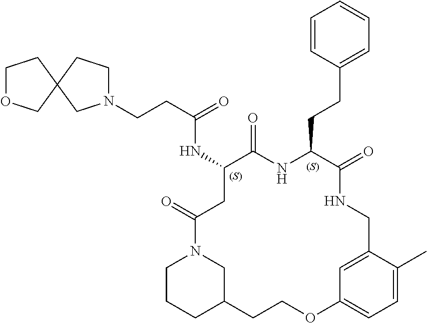 | N-((9S,12S)-5⁴-methyl-8,11,14-trioxo-9- phenethyl-4-oxa-7,10-diaza-1(3,1)-piperidina- 5(1,3)-benzenacyclotetradecaphane-12-yl)-3- (2-oxa-7-azaspiro[4.4]nonan-7- yl)propanamide |
| 641 | 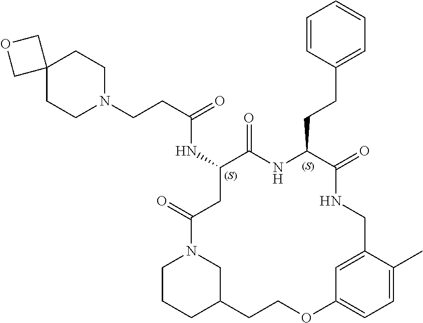 | N-((9S,12S)-5⁴-methyl-8,11,14-trioxo-9- phenethyl-4-oxa-7,10-diaza-1(3,1)-piperidina- 5(1,3)-benzenacyclotetradecaphane-12-yl)-3- (2-oxa-7-azaspiro[3.5]nonan-7- yl)propanamide |
| 642 | 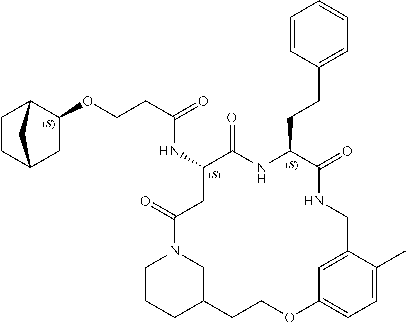 | 3-(((1S,2S,4R)-bicyclo[2.2.1]heptan-2-yl)oxy)- N-((9S,12S)-5⁴-methyl-8,11,14-trioxo-9- phenethyl-4-oxa-7,10-diaza-1(3,1)-piperidina- 5(1,3)-benzenacyclotetradecaphane-12- yl)propanamide |
| 643 | 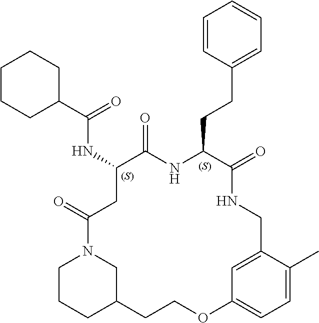 | N-((9S,12S)-5⁴-methyl-8,11,14-trioxo-9- phenethyl-4-oxa-7,10-diaza-1(3,1)-piperidina- 5(1,3)-benzenacyclotetradecaphane-12- yl)cyclohexanecarboxamide |
| 644 | 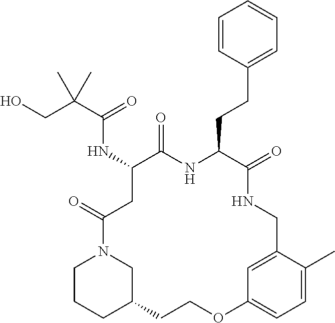 [SMILES: Cc1ccc2cc1CNC(=O)[C@H](CCc1ccccc1)NC(=O)[C@@H](NC(=O)C(C)(C)CO)CC(=O)N1CCC[C@@H](CCO2)C1] | 3-hydroxy-2,2-dimethyl-N-((13S,9S,12S)-5⁴- methyl-8,11,14-trioxo-9-phenethyl-4-oxa- 7,10-diaza-1(3,1)-piperidina-5(1,3)- benzenacyclotetradecaphane-12- yl)propanamide |
| 645 | 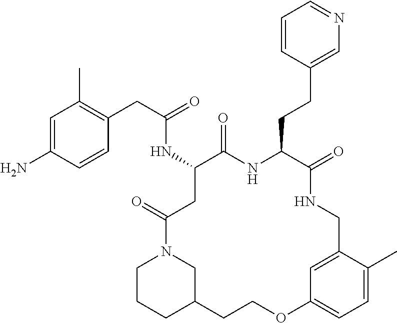 [SMILES: Cc1ccc2cc1CNC(=O)[C@H](CCc1cccnc1)NC(=O)[C@@H](NC(=O)Cc1ccc(N)cc1C)CC(=O)N1CCCC(CCO2)C1] | 2-(4-amino-2-methylphenyl)-N-((9S,12S)-5⁴- methyl-8,11,14-trioxo-9-(2-(pyridin-3-yl)ethyl)- 4-oxa-7,10-diaza-1(3,1)-piperidina-5(1,3)- benzenacyclotetradecaphane-12- yl)acetamide |
| 646 | 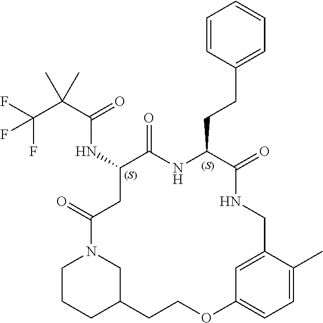 | 3,3,3-trifluoro-2,2-dimethyl-N-((9S,12S)-5⁴- methyl-8,11,14-trioxo-9-phenethyl-4-oxa- 7,10-diaza-1(3,1)-piperidina-5(1,3)- benzenacyclotetradecaphane-12- yl)propanamide |
| 647 | 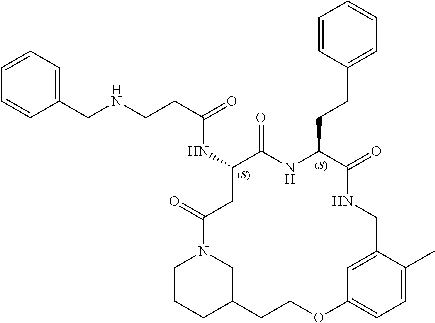 | 3-(benzylamino)-N-((9S,12S)-5⁴-methyl- 8,11,14-trioxo-9-phenethyl-4-oxa-7,10-diaza- 1(3,1)-piperidina-5(1,3)- benzenacyclotetradecaphane-12- yl)propanamide |
| 648 | 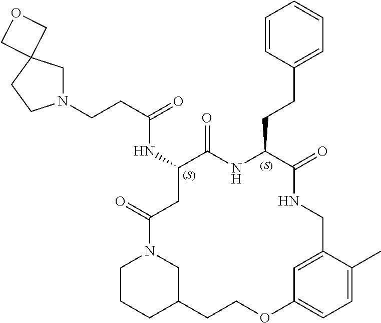 | N-((9S,12S)-5⁴-methyl-8,11,14-trioxo-9- phenethyl-4-oxa-7,10-diaza-1(3,1)-piperidina- 5(1,3)-benzenacyclotetradecaphane-12-yl)-3- (2-oxa-6-azaspiro[3.4]octan-6- yl)propanamide |
| 649 | 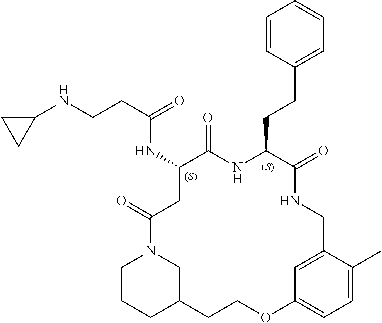 | 3-(cyclopropylamino)-N-((9S,12S)-5⁴-methyl- 8,11,14-trioxo-9-phenethyl-4-oxa-7,10-diaza- 1(3,1)-piperidina-5(1,3)- benzenacyclotetradecaphane-12- yl)propanamide |
| 650 | 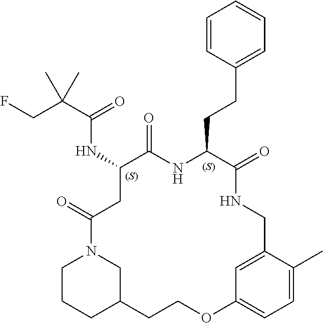 | 3-fluoro-2,2-dimethyl-N-((9S,12S)-5⁴-methyl- 8,11,14-trioxo-9-phenethyl-4-oxa-7,10-diaza- 1(3,1)-piperidina-5(1,3)- benzenacyclotetradecaphane-12- yl)propanamide |
| 651 | 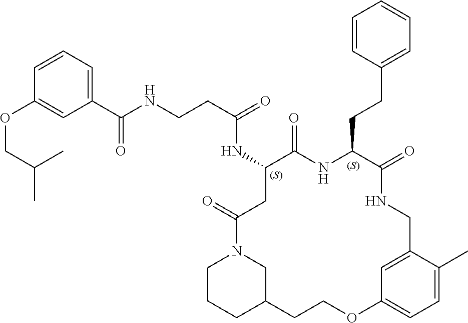 | 3-isobutoxy-N-(3-(((9S,12S)-5⁴-methyl- 8,11,14-trioxo-9-phenethyl-4-oxa-7,10-diaza- 1(3,1)-piperidina-5(1,3)- benzenacyclotetradecaphane-12-yl)amino)-3- oxopropyl)benzamide |
| 652 | 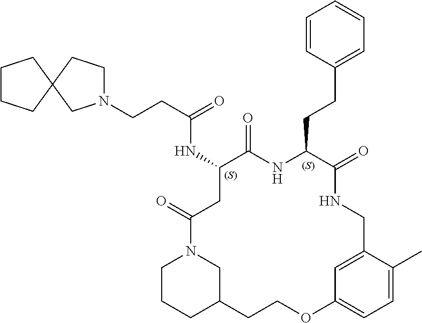 | N-((9S,12S)-5⁴-methyl-8,11,14-trioxo-9- phenethyl-4-oxa-7,10-diaza-1(3,1)-piperidina- 5(1,3)-benzenacyclotetradecaphane-12-yl)-3- (2-azaspiro[4.4]nonan-2-yl)propanamide |
| 653 | 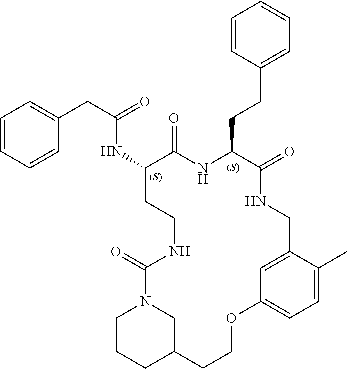 | N-((9S,12S)-5⁴-methyl-8,11,16-trioxo-9- phenethyl-4-oxa-7,10,15-triaza-1(3,1)- piperidina-5(1,3)- benzenacyclohexadecaphane-12-yl)-2- phenylacetamide |
| 654 | 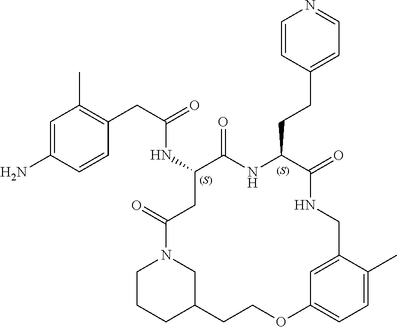 | 2-(4-amino-2-methylphenyl)-N-((9S,12S)-5⁴- methyl-8,11,14-trioxo-9-(2-(pyridin-4-yl)ethyl)- 4-oxa-7,10-diaza-1(3,1)-piperidina-5(1,3)- benzenacyclotetradecaphane-12- yl)acetamide |
| 655 | 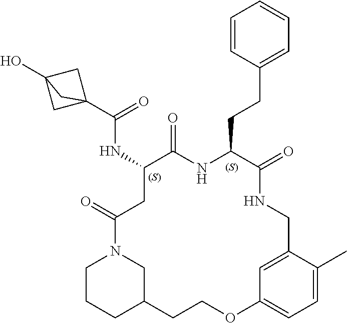 | 3-hydroxy-N-((9S,12S)-5⁴-methyl-8,11,14- trioxo-9-phenethyl-4-oxa-7,10-diaza-1(3,1)- piperidina-5(1,3)- benzenacyclotetradecaphane-12- yl)bicyclo[1.1.1]pentane-1-carboxamide |
| 656 | 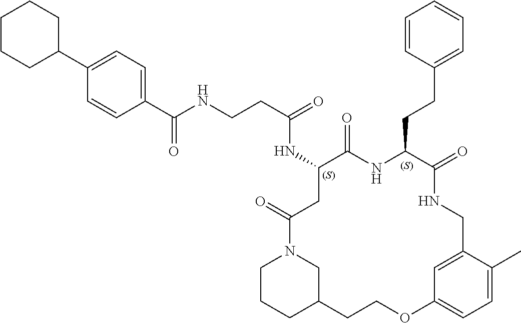 | 4-cyclohexyl-N-(3-(((9S,12S)-5⁴-methyl- 8,11,14-trioxo-9-phenethyl-4-oxa-7,10-diaza- 1(3,1)-piperidina-5(1,3)- benzenacyclotetradecaphane-12-yl)amino)-3- oxopropyl)benzamide |
| 657 | 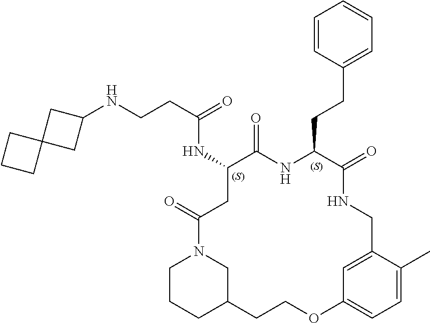 | N-((9S,12S)-5⁴-methyl-8,11,14-trioxo-9- phenethyl-4-oxa-7,10-diaza-1(3,1)-piperidina- 5(1,3)-benzenacyclotetradecaphane-12-yl)-3- (spiro[3.3]heptan-2-ylamino)propanamide |
| 658 | 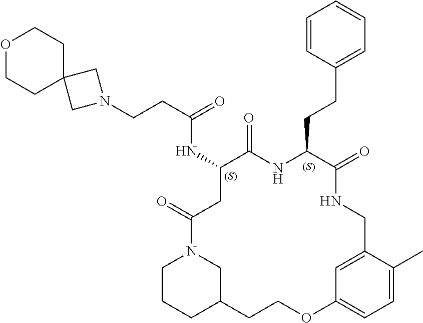 | N-((9S,12S)-5⁴-methyl-8,11,14-trioxo-9- phenethyl-4-oxa-7,10-diaza-1(3,1)-piperidina- 5(1,3)-benzenacyclotetradecaphane-12-yl)-3- (7-oxa-2-azaspiro[3.5]nonan-2- yl)propanamide |
| 659 | 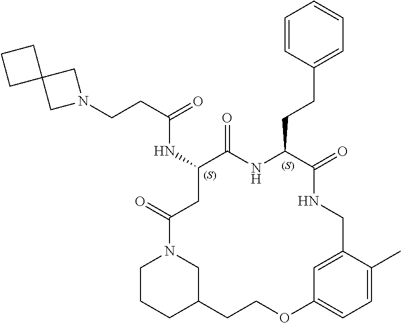 | N-((9S,12S)-5⁴-methyl-8,11,14-trioxo-9- phenethyl-4-oxa-7,10-diaza-1(3,1)-piperidina- 5(1,3)-benzenacyclotetradecaphane-12-yl)-3- (2-azaspiro[3.3]heptan-2-yl)propanamide |
| 660 | 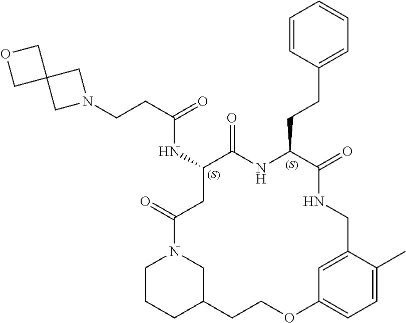 | N-((9S,12S)-5⁴-methyl-8,11,14-trioxo-9- phenethyl-4-oxa-7,10-diaza-1(3,1)-piperidina- 5(1,3)-benzenacyclotetradecaphane-12-yl)-3- (2-oxa-6-azaspiro[3.3]heptan-6- yl)propanamide |
| 661 | 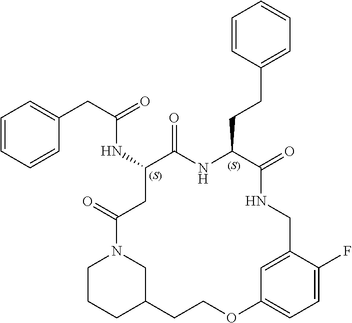 | N-((9S,12S)-5⁴-fluoro-8,11,14-trioxo-9- phenethyl-4-oxa-7,10-diaza-1(3,1)-piperidina- 5(1,3)-benzenacyclotetradecaphane-12-yl)-2- phenylacetamide |
| 662 | 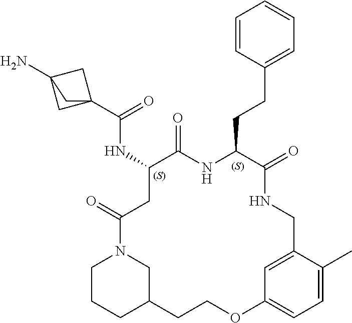 | 3-amino-N-((9S,12S)-5⁴-methyl-8,11,14- trioxo-9-phenethyl-4-oxa-7,10-diaza-1(3,1)- piperidina-5(1,3)- benzenacyclotetradecaphane-12- yl)bicyclo[1.1.1]pentane-1-carboxamide |
| 663 | 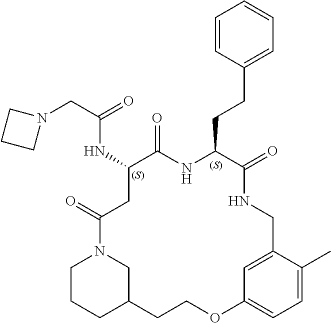 | 2-(azetidin-1-yl)-N-((9S,12S)-5⁴-methyl- 8,11,14-trioxo-9-phenethyl-4-oxa-7,10-diaza- 1(3,1)-piperidina-5(1,3)- benzenacyclotetradecaphane-12- yl)acetamide |
| 664 | 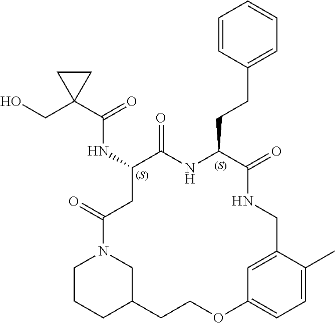 | 1-(hydroxymethyl)-N-((9S,12S)-5⁴-methyl- 8,11,14-trioxo-9-phenethyl-4-oxa-7,10-diaza- 1(3,1)-piperidina-5(1,3)- benzenacyclotetradecaphane-12- yl)cyclopropane-1-carboxamide |
| 665 | 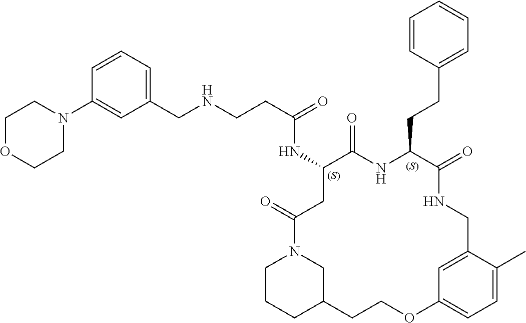 | N-((9S,12S)-5⁴-methyl-8,11,14-trioxo-9- phenethyl-4-oxa-7,10-diaza-1(3,1)-piperidina- 5(1,3)-benzenacyclotetradecaphane-12-yl)-3- ((3-morpholinobenzyl)amino)propanamide |
| 666 | 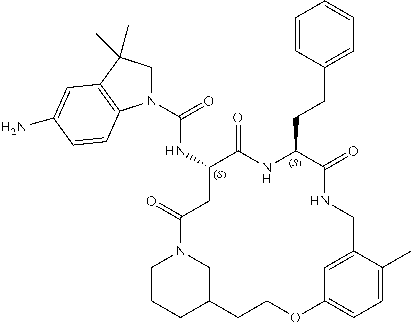 | 5-amino-3,3-dimethyl-N-((9S,12S)-5⁴-methyl- 8,11,14-trioxo-9-phenethyl-4-oxa-7,10-diaza- 1(3,1)-piperidina-5(1,3)- benzenacyclotetradecaphane-12-yl)indoline- 1-carboxamide |
| 667 | 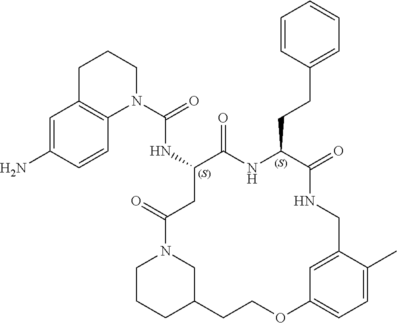 | 6-amino-N-((9S,12S)-5⁴-methyl-8,11,14- trioxo-9-phenethyl-4-oxa-7,10-diaza-1(3,1)- piperidina-5(1,3)- benzenacyclotetradecaphane-12-yl)-3,4- dihydroquinoline-1(2H)-carboxamide |
| 668 | 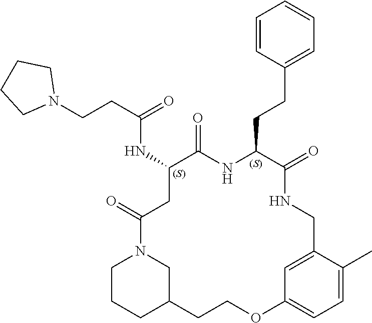 | N-((9S,12S)-5⁴-methyl-8,11,14-trioxo- 9-phenethyl-4-oxa-7,10-diaza-1(3,1)- piperidina-5(1,3)- benzenacyclotetradecaphane-12-yl)-3- (pyrrolidin-1-yl)propanamide |
| 669 | 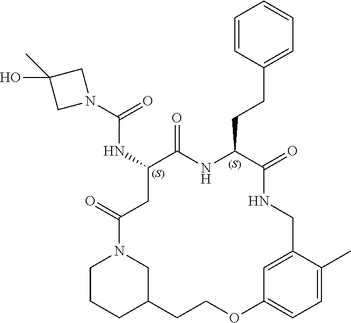 | 3-hydroxy-3-methyl-N-((9S,12S)-5⁴-methyl- 8,11,14-trioxo-9-phenethyl-4-oxa-7,10-diaza- 1(3,1)-piperidina-5(1,3)- benzenacyclotetradecaphane-12-yl)azetidine- 1-carboxamide |
| 670 | 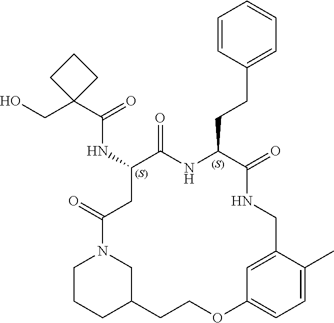 | 1-(hydroxymethyl)-N-((9S,12S)-5⁴-methyl- 8,11,14-trioxo-9-phenethyl-4-oxa-7,10-diaza- 1(3,1)-piperidina-5(1,3)- benzenacyclotetradecaphane-12- yl)cyclobutane-1-carboxamide |
| 671 | 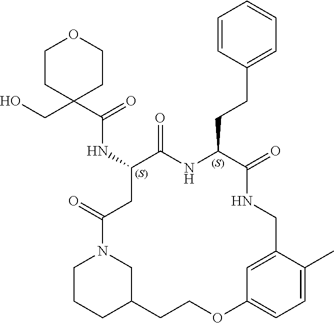 | 4-(hydroxymethyl)-N-((9S,12S)-5⁴-methyl- 8,11,14-trioxo-9-phenethyl-4-oxa-7,10-diaza- 1(3,1)-piperidina-5(1,3)- benzenacyclotetradecaphane-12- yl)tetrahydro-2H-pyran-4-carboxamide |
| 672 | 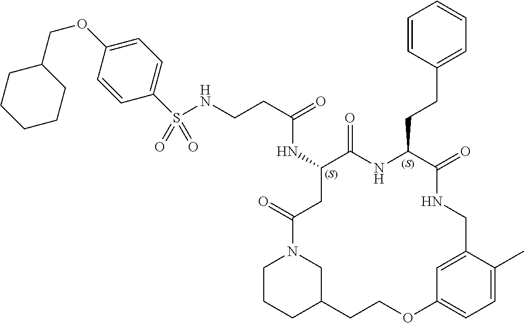 | 3-((4- (cyclohexylmethoxy)phenyl)sulfonamido)-N- ((9S,12S)-5⁴-methyl-8,11,14-trioxo-9- phenethyl-4-oxa-7,10-diaza-1(3,1)-piperidina- 5(1,3)-benzenacyclotetradecaphane-12- yl)propanamide |
| 673 | 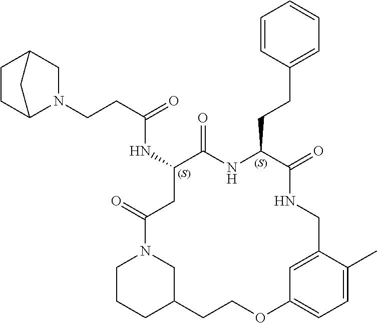 | 3-(2-azabicyclo[2.2.1]heptan-2-yl)-N- ((9S,12S)-5⁴-methyl-8,11,14-trioxo-9- phenethyl-4-oxa-7,10-diaza-1(3,1)-piperidina- 5(1,3)-benzenacyclotetradecaphane-12- yl)propanamide |
| 674 | 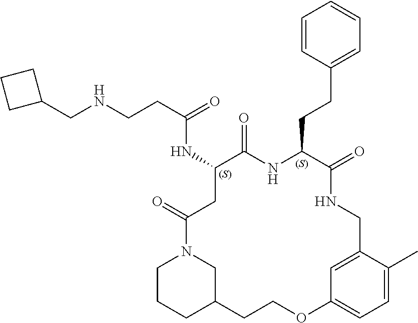 | 3-((cyclobutylmethyl)amino)-N-((9S,12S)-5⁴- methyl-8,11,14-trioxo-9-phenethyl-4-oxa- 7,10-diaza-1(3,1)-piperidina-5(1,3)- benzenacyclotetradecaphane-12- yl)propanamide |
| 675 | 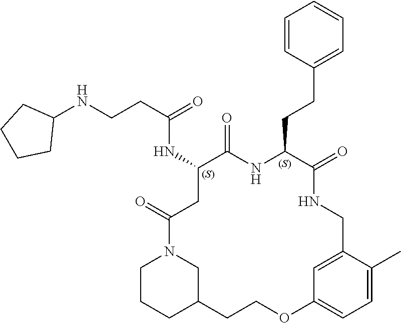 | 3-(cyclopentylamino)-N-((9S,12S)-5⁴-methyl- 8,11,14-trioxo-9-phenethyl-4-oxa-7,10-diaza- 1(3,1)-piperidina-5(1,3)- benzenacyclotetradecaphane-12- yl)propanamide |
| 676 | 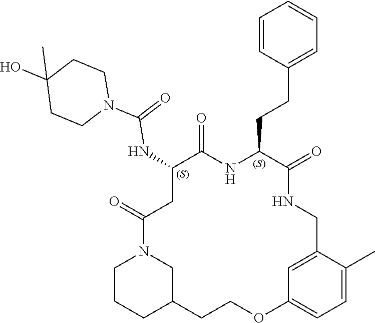 | 4-hydroxy-4-methyl-N-((9S,12S)-5⁴-methyl- 8,11,14-trioxo-9-phenethyl-4-oxa-7,10-diaza- 1(3,1)-piperidina-5(1,3)- benzenacyclotetradecaphane-12- yl)piperidine-1-carboxamide |
| 677 | 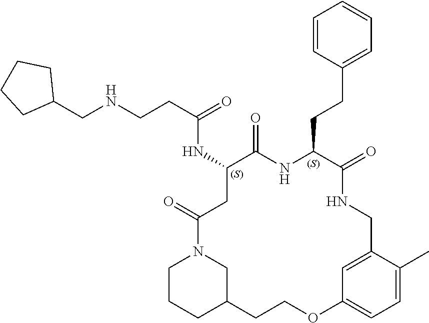 | 3-((cyclopentylmethyl)amino)-N-((9S,12S)-5⁴- methyl-8,11,14-trioxo-9-phenethyl-4-oxa- 7,10-diaza-1(3,1)-piperidina-5(1,3)- benzenacyclotetradecaphane-12- yl)propanamide |
| 678 | 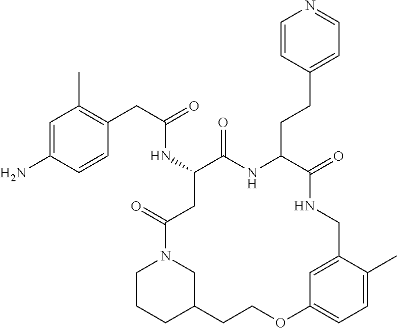 [SMILES: Cc1ccc2cc1CNC(=O)C(CCc1ccncc1)NC(=O)[C@@H](NC(=O)Cc1ccc(N)cc1C)CC(=O)N1CCCC(CCO2)C1] | 2-(4-amino-2-methylphenyl)-N-((12S)-5⁴- methyl-8,11,14-trioxo-9-(2-(pyridin-4-yl)ethyl)- 4-oxa-7,10-diaza-1(3,1)-piperidina-5(1,3)- benzenacyclotetradecaphane-12- yl)acetamide |
| 679 | 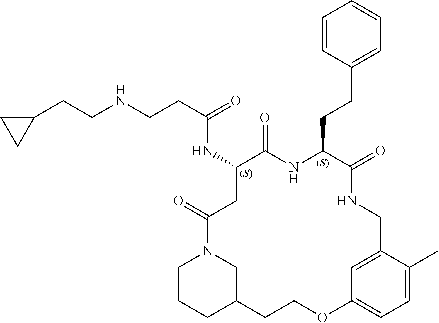 | 3-((2-cyclopropylethyl)amino)-N-((9S,12S)-5⁴- methyl-8,11,14-trioxo-9-phenethyl-4-oxa- 7,10-diaza-1(3,1)-piperidina-5(1,3)- benzenacyclotetradecaphane-12- yl)propanamide |
| 680 | 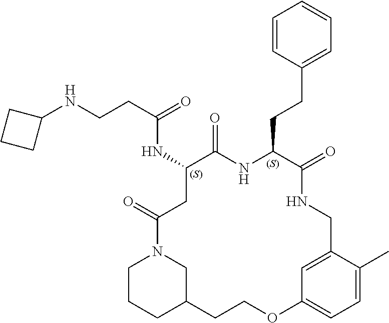 | 3-(cyclobutylamino)-N-((9S,12S)-5⁴-methyl- 8,11,14-trioxo-9-phenethyl-4-oxa-7,10-diaza- 1(3,1)-piperidina-5(1,3)- benzenacyclotetradecaphane-12- yl)propanamide |
| 681 | 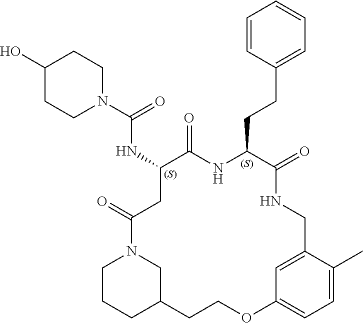 | 4-hydroxy-N-((9S,12S)-5⁴-methyl-8,11,14- trioxo-9-phenethyl-4-oxa-7,10-diaza-1(3,1)- piperidina-5(1,3)- benzenacyclotetradecaphane-12- yl)piperidine-1-carboxamide |
| 682 | 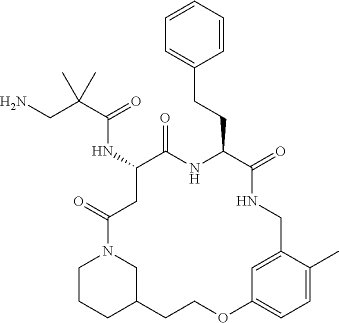 [SMILES: Cc1ccc2cc1CNC(=O)[C@H](CCc1ccccc1)NC(=O)[C@@H](NC(=O)C(C)(C)CN)CC(=O)N1CCCC(CCO2)C1] | 3-amino-2,2-dimethyl-N-((9S,12S)-5⁴-methyl- 8,11,14-trioxo-9-phenethyl-4-oxa-7,10-diaza- 1(3,1)-piperidina-5(1,3)- benzenacyclotetradecaphane-12- yl)propanamide |
| 683 | 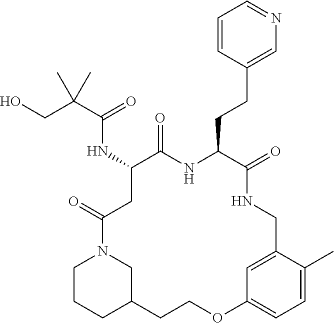 [SMILES: Cc1ccc2cc1CNC(=O)[C@H](CCc1cccnc1)NC(=O)[C@@H](NC(=O)C(C)(C)CO)CC(=O)N1CCCC(CCO2)C1] | 3-hydroxy-2,2-dimethyl-N-((9S,12S)-5⁴- methyl-8,11,14-trioxo-9-(2-(pyridin-3-yl)ethyl)- 4-oxa-7,10-diaza-1(3,1)-piperidina-5(1,3)- benzenacyclotetradecaphane-12- yl)propanamide |
| 684 | 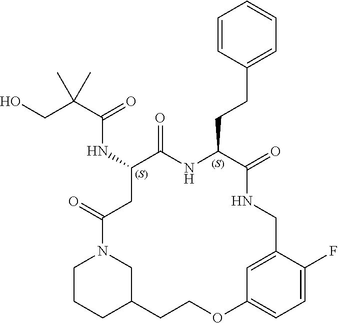 | N-((9S,12S)-5⁴-fluoro-8,11,14-trioxo-9- phenethyl-4-oxa-7,10-diaza-1(3,1)-piperidina- 5(1,3)-benzenacyclotetradecaphane-12-yl)-3- hydroxy-2,2-dimethylpropanamide |
| 685 | 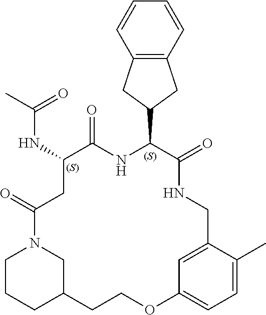 | N-((9S,12S)-9-(2,3-dihydro-1H-inden-2-yl)-5⁴- methyl-8,11,14-trioxo-4-oxa-7,10-diaza- 1(3,1)-piperidina-5(1,3)- benzenacyclotetradecaphane-12- yl)acetamide |
| 686 | 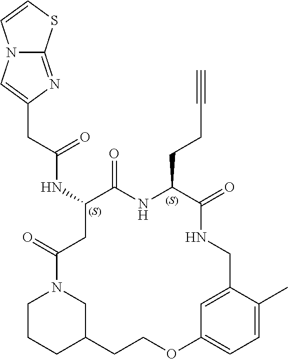 | N-((9S,12S)-9-(but-3-yn-1-yl)-5⁴-methyl- 8,11,14-trioxo-4-oxa-7,10-diaza-1(3,1)- piperidina-5(1,3)- benzenacyclotetradecaphane-12-yl)-2- (imidazo[2,1-b]thiazol-6-yl)acetamide |
| 687 | 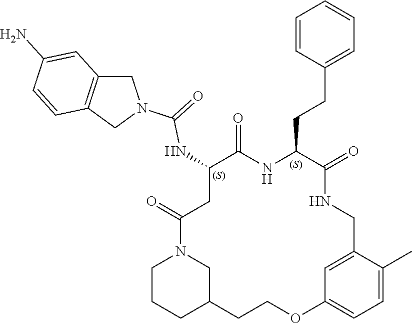 | 5-amino-N-((9S,12S)-5⁴-methyl-8,11,14- trioxo-9-phenethyl-4-oxa-7,10-diaza-1(3,1)- piperidina-5(1,3)- benzenacyclotetradecaphane-12- yl)isoindoline-2-carboxamide |
| 688 | 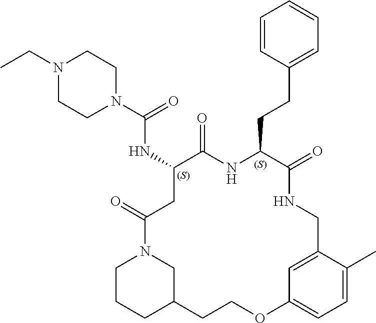 | 4-ethyl-N-((9S,12S)-5⁴-methyl-8,11,14-trioxo- 9-phenethyl-4-oxa-7,10-diaza-1(3,1)- piperidina-5(1,3)- benzenacyclotetradecaphane-12- yl)piperazine-1-carboxamide |
| 689 | 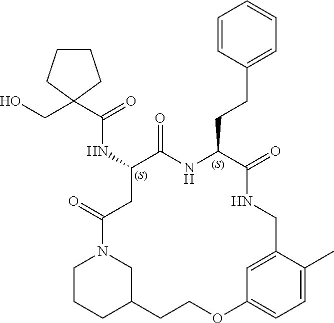 | 1-(hydroxymethyl)-N-((9S,12S)-5⁴-methyl- 8,11,14-trioxo-9-phenethyl-4-oxa-7,10-diaza- 1(3,1)-piperidina-5(1,3)- benzenacyclotetradecaphane-12- yl)cyclopentane-1-carboxamide |
| 690 | 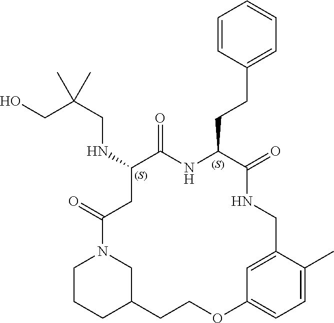 | (9S,12S)-12-((3-hydroxy-2,2- dimethylpropyl)amino)-5⁴-methyl-9-phenethyl- 4-oxa-7,10-diaza-1(3,1)-piperidina-5(1,3)- benzenacyclotetradecaphane-8,11,14-trione |
| 691 | 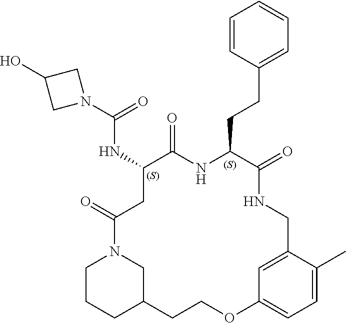 | 3-hydroxy-N-((9S,12S)-5⁴-methyl-8,11,14- trioxo-9-phenethyl-4-oxa-7,10-diaza-1(3,1)- piperidina-5(1,3)- benzenacyclotetradecaphane-12-yl)azetidine- 1-carboxamide |
| 692 | 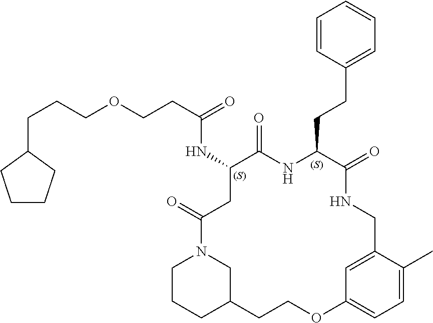 | 3-(3-cyclopentylpropoxy)-N-((9S,12S)-5⁴- methyl-8,11,14-trioxo-9-phenethyl-4-oxa- 7,10-diaza-1(3,1)-piperidina-5(1,3)- benzenacyclotetradecaphane-12- yl)propanamide |
| 693 | 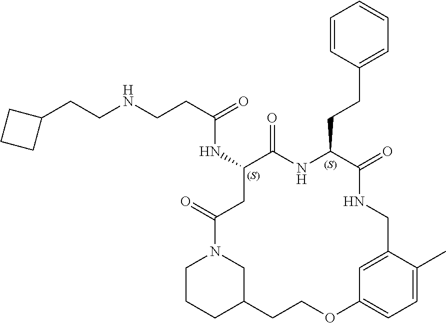 | 3-((2-cyclobutylethyl)amino)-N-((9S,12S)-5⁴- methyl-8,11,14-trioxo-9-phenethyl-4-oxa- 7,10-diaza-1(3,1)-piperidina-5(1,3)- benzenacyclotetradecaphane-12- yl)propanamide |
| 694 | 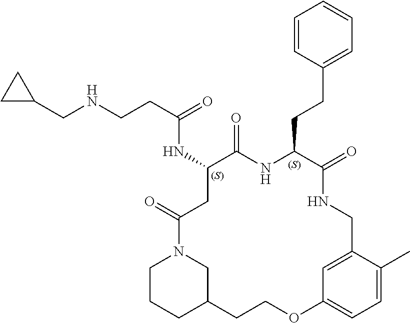 | 3-((cyclopropylmethyl)amino)-N-((9S,12S)-5⁴- methyl-8,11,14-trioxo-9-phenethyl-4-oxa- 7,10-diaza-1(3,1)-piperidina-5(1,3)- benzenacyclotetradecaphane-12- yl)propanamide |
| 695 | 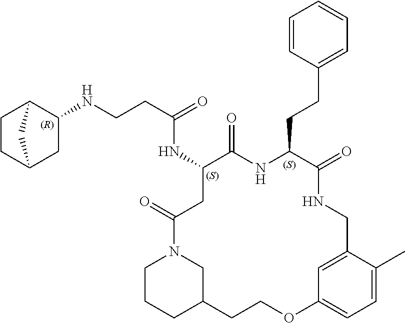 | 3-(((1R,2R,4S)-bicyclo[2.2.1]heptan-2- yl)amino)-N-((9S,12S)-5⁴-methyl-8,11,14- trioxo-9-phenethyl-4-oxa-7,10-diaza-1(3,1)- piperidina-5(1,3)- benzenacyclotetradecaphane-12- yl)propanamide |
| 696 | 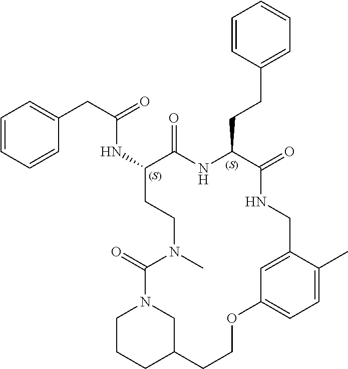 | N-((9S,12S)-5⁴,15-dimethyl-8,11,16-trioxo-9- phenethyl-4-oxa-7,10,15-triaza-1(3,1)- piperidina-5(1,3)- benzenacyclohexadecaphane-12-yl)-2- phenylacetamide |
| 697 | 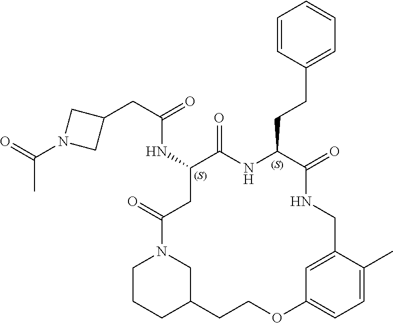 | 2-(1-acetylazetidin-3-yl)-N-((9S,12S)-5⁴- methyl-8,11,14-trioxo-9-phenethyl-4-oxa- 7,10-diaza-1(3,1)-piperidina-5(1,3)- benzenacyclotetradecaphane-12- yl)acetamide |
| 698 | 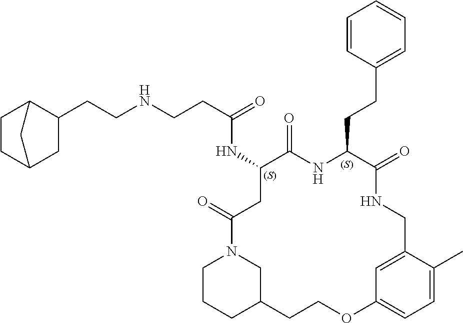 | 3-((2-(bicyclo[2.2.1]heptan-2-yl)ethyl)amino)- N-((9S,12S)-5⁴-methyl-8,11,14-trioxo-9- phenethyl-4-oxa-7,10-diaza-1(3,1)-piperidina- 5(1,3)-benzenacyclotetradecaphane-12- yl)propanamide |
| 699 | 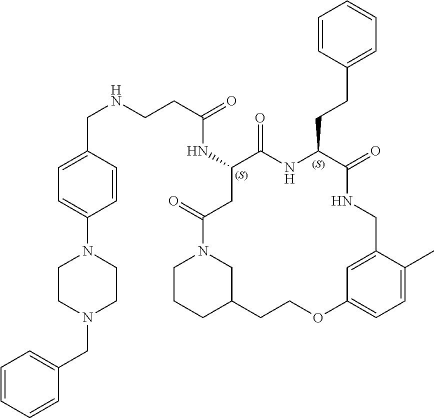 | 3-((4-(4-benzylpiperazin-1-yl)benzyl)amino)- N-((9S,12S)-5⁴-methyl-8,11,14-trioxo-9- phenethyl-4-oxa-7,10-diaza-1(3,1)-piperidina- 5(1,3)-benzenacyclotetradecaphane-12- yl)propanamide |
| 700 | 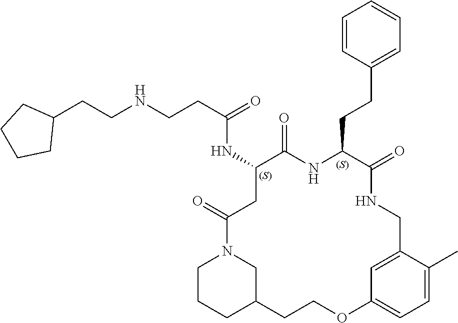 | 3-((2-cyclopentylethyl)amino)-N-((9S,12S)-5⁴- methyl-8,11,14-trioxo-9-phenethyl-4-oxa- 7,10-diaza-1(3,1)-piperidina-5(1,3)- benzenacyclotetradecaphane-12- yl)propanamide |
| 701 | 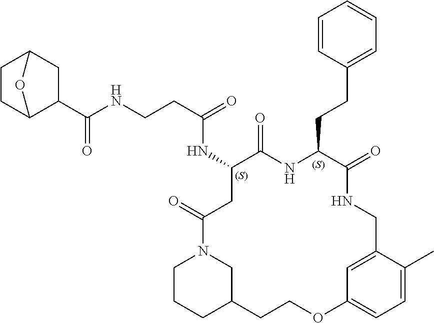 | N-(3-(((9S,12S)-5⁴-methyl-8,11,14-trioxo-9- phenethyl-4-oxa-7,10-diaza-1(3,1)-piperidina- 5(1,3)-benzenacyclotetradecaphane-12- yl)amino)-3-oxopropyl)-7- oxabicyclo[2.2.1]heptane-2-carboxamide |
| 702 | 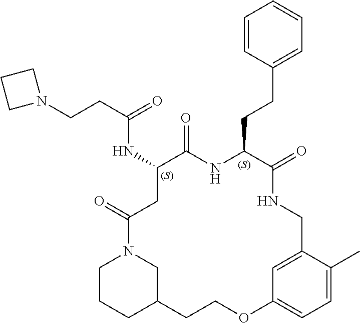 | 3-(azetidin-1-yl)-N-((9S,12S)-5⁴-methyl- 8,11,14-trioxo-9-phenethyl-4-oxa-7,10-diaza- 1(3,1)-piperidina-5(1,3)- benzenacyclotetradecaphane-12- yl)propanamide |
| 703 | 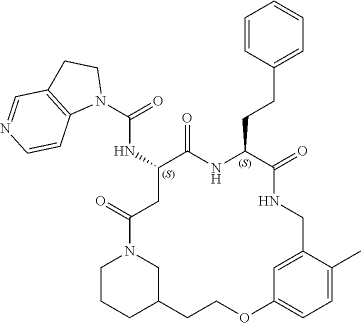 | N-((9S,12S)-5⁴-methyl-8,11,14-trioxo-9- phenethyl-4-oxa-7,10-diaza-1(3,1)-piperidina- 5(1,3)-benzenacyclotetradecaphane-12-yl)- 2,3-dihydro-1H-pyrrolo[3,2-c]pyridine-1- carboxamide |
| 704 | 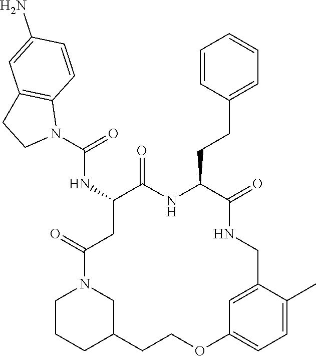 [SMILES: Cc1ccc2cc1CNC(=O)[C@H](CCc1ccccc1)NC(=O)[C@@H](NC(=O)N1CCc3cc(N)ccc31)CC(=O)N1CCCC(CCO2)C1] | 5-amino-N-((9S,12S)-5⁴-methyl-8,11,14- trioxo-9-phenethyl-4-oxa-7,10-diaza-1(3,1)- piperidina-5(1,3)- benzenacyclotetradecaphane-12-yl)indoline- 1-carboxamide |
| 705 | 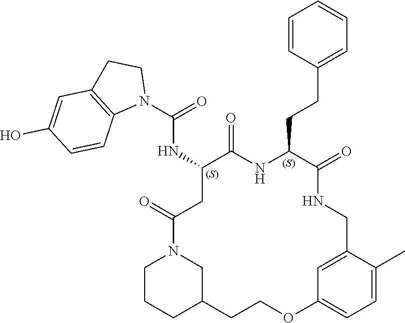 | 5-hydroxy-N-((9S,12S)-5⁴-methyl-8,11,14- trioxo-9-phenethyl-4-oxa-7,10-diaza-1(3,1)- piperidina-5(1,3)- benzenacyclotetradecaphane-12-yl)indoline- 1-carboxamide |
| 706 | 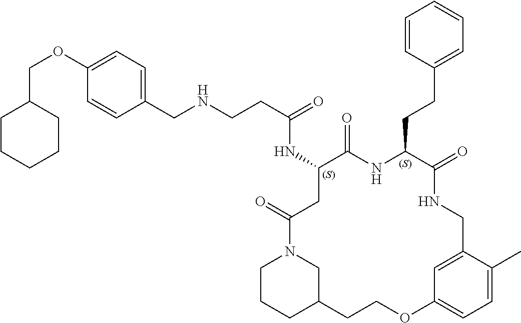 | 3-((4-(cyclohexylmethoxy)benzyl)amino)-N- ((9S,12S)-5⁴-methyl-8,11,14-trioxo-9- phenethyl-4-oxa-7,10-diaza-1(3,1)-piperidina- 5(1,3)-benzenacyclotetradecaphane-12- yl)propanamide |
| 707 | 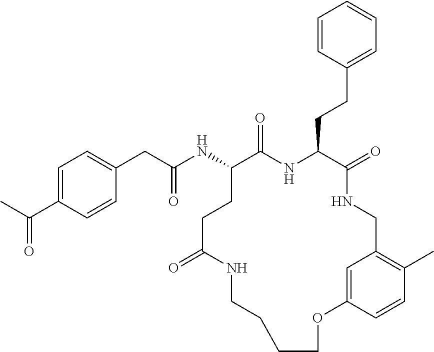 [SMILES: CC(=O)c1ccc(CC(=O)N[C@H]2CCC(=O)NCCCCOc3ccc(C)c(c3)CNC(=O)[C@H](CCc3ccccc3)NC2=O)cc1] | 2-(4-acetylphenyl)-N-((11S,14S)-1⁴-methyl- 8,12,15-trioxo-1⁴-phenethyl-2-oxa-7,13,16- triaza-1(1,3)-benzenacycloheptadecaphane- 11-yl)acetamide |
| 708 | 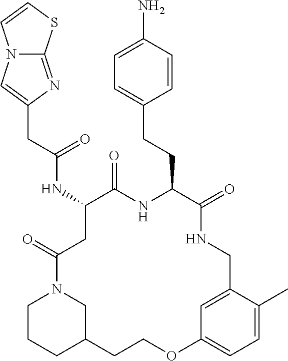 [SMILES: Cc1ccc2cc1CNC(=O)[C@H](CCc1ccc(N)cc1)NC(=O)[C@@H](NC(=O)Cc1cn3ccsc3n1)CC(=O)N1CCCC(CCO2)C1] | N-((9S,12S)-9-(4-aminophenethyl)-5⁴-methyl- 8,11,14-trioxo-4-oxa-7,10-diaza-1(3,1)- piperidina-5(1,3)- benzenacyclotetradecaphane-12-yl)-2- (imidazo[2,1-b]thiazol-6-yl)acetamide |
| 709 | 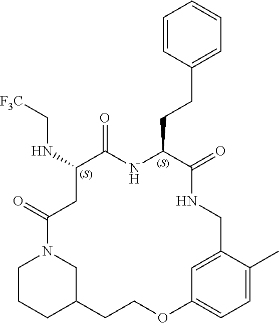 | (9S,12S)-5⁴-methyl-9-phenethyl-12-((2,2,2- trifluoroethyl)amino)-4-oxa-7,10-diaza-1(3,1)- piperidina-5(1,3)- benzenacyclotetradecaphane-8,11,14-trione |
| 710 | 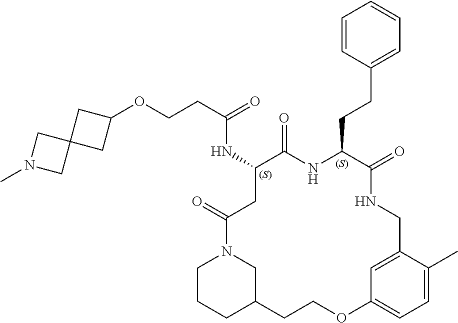 | 3-((2-methyl-2-azaspiro[3.3]heptan-6-yl)oxy)- N-((9S,12S)-5⁴-methyl-8,11,14-trioxo-9- phenethyl-4-oxa-7,10-diaza-1(3,1)-piperidina- 5(1,3)-benzenacyclotetradecaphane-12- yl)propanamide |
| 711 | 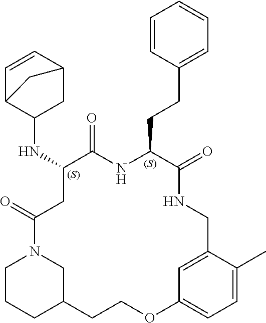 | (9S,12S)-12-(bicyclo[2.2.1]hept-5-en-2- ylamino)-5⁴-methyl-9-phenethyl-4-oxa-7,10- diaza-1(3,1)-piperidina-5(1,3)- benzenacyclotetradecaphane-8,11,14-trione |
| 712 | 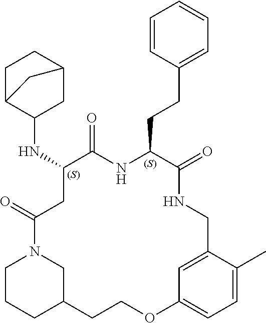 | (9S,12S)-12-(bicyclo[2.2.1]heptan-2- ylamino)-5⁴-methyl-9-phenethyl-4-oxa-7,10- diaza-1(3,1)-piperidina-5(1,3)- benzenacyclotetradecaphane-8,11,14-trione |
| 713 | 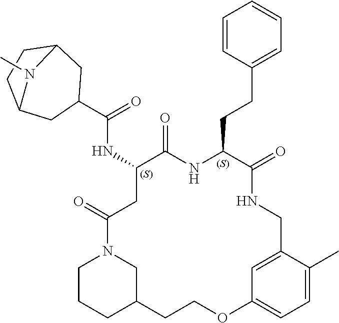 | 8-methyl-N-((9S,12S)-5⁴-methyl-8,11,14- trioxo-9-phenethyl-4-oxa-7,10-diaza-1(3,1)- piperidina-5(1,3)- benzenacyclotetradecaphane-12-yl)-8- azabicyclo[3.2.1]octane-3-carboxamide |
| 714 | 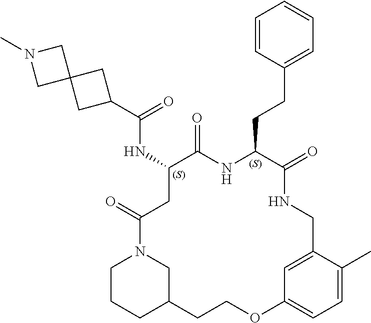 | 2-methyl-N-((9S,12S)-5⁴-methyl-8,11,14- trioxo-9-phenethyl-4-oxa-7,10-diaza-1(3,1)- piperidina-5(1,3)- benzenacyclotetradecaphane-12-yl)-2- azaspiro[3.3]heptane-6-carboxamide |
| 715 | 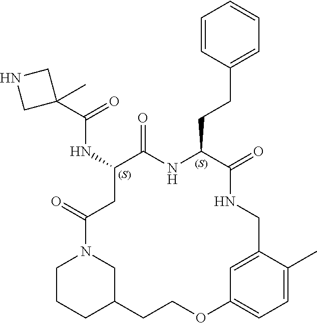 | 3-methyl-N-((9S,12S)-5⁴-methyl-8,11,14- trioxo-9-phenethyl-4-oxa-7,10-diaza-1(3,1)- piperidina-5(1,3)- benzenacyclotetradecaphane-12-yl)azetidine- 3-carboxamide |
| 716 | 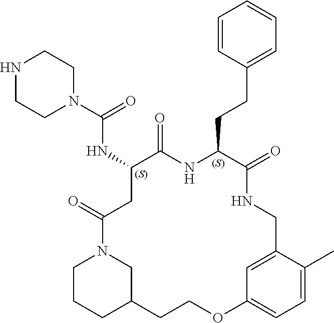 | N-((9S,12S)-5⁴-methyl-8,11,14-trioxo-9- phenethyl-4-oxa-7,10-diaza-1(3,1)-piperidina- 5(1,3)-benzenacyclotetradecaphane-12- yl)piperazine-1-carboxamide |
| 717 | 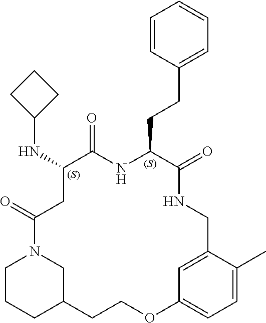 | (9S,12S)-12-(cyclobutylamino)-5⁴-methyl-9- phenethyl-4-oxa-7,10-diaza-1(3,1)-piperidina- 5(1,3)-benzenacyclotetradecaphane-8,11,14- trione |
| 718 | 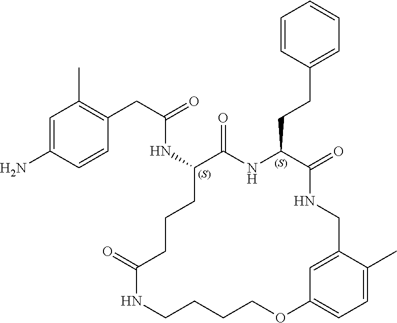 | 2-(4-amino-2-methylphenyl)-N-((12S,15S)-1⁴- methyl-8,13,16-trioxo-15-phenethyl-2-oxa- 7,14,17-triaza-1(1,3)- benzenacyclooctadecaphane-12- yl)acetamide |
| 719 | 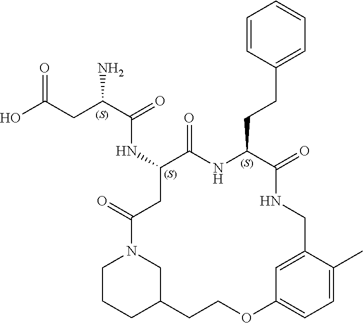 | (3S)-3-amino-4-(((9S,12S)-5⁴-methyl-8,11,14- trioxo-9-phenethyl-4-oxa-7,10-diaza-1(3,1)- piperidina-5(1,3)- benzenacyclotetradecaphane-12-yl)amino)-4- oxobutanoic acid |
| 720 | 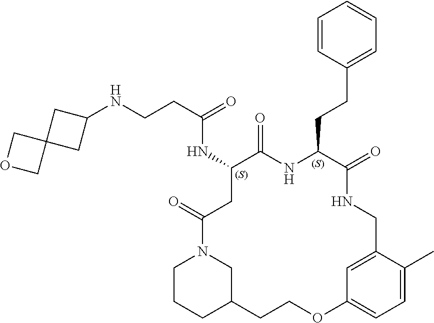 | 3-((2-oxaspiro[3.3]heptan-6-yl)amino)-N- ((9S,12S)-5⁴-methyl-8,11,14-trioxo-9- phenethyl-4-oxa-7,10-diaza-1(3,1)-piperidina- 5(1,3)-benzenacyclotetradecaphane-12- yl)propanamide |
| 721 | 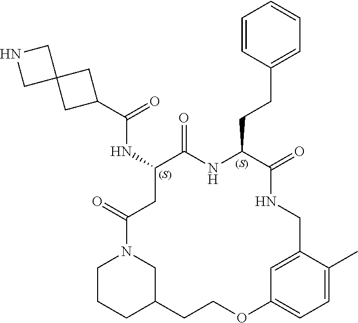 | N-((9S,12S)-5⁴-methyl-8,11,14-trioxo-9- phenethyl-4-oxa-7,10-diaza-1(3,1)-piperidina- 5(1,3)-benzenacyclotetradecaphane-12-yl)-2- azaspiro[3.3]heptane-6-carboxamide |
| 722 | 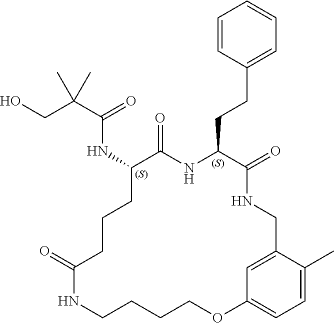 | 3-hydroxy-2,2-dimethyl-N-((12S,15S)-1⁴- methyl-8,13,16-trioxo-15-phenethyl-2-oxa- 7,14,17-triaza-1(1,3)- benzenacyclooctadecaphane-12- yl)propanamide |
| 723 | 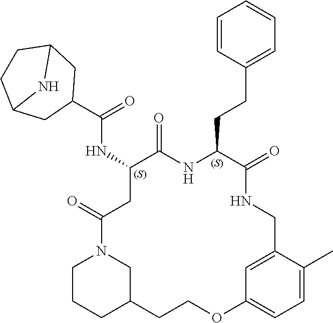 | N-((9S,12S)-5⁴-methyl-8,11,14-trioxo-9- phenethyl-4-oxa-7,10-diaza-1(3,1)-piperidina- 5(1,3)-benzenacyclotetradecaphane-12-yl)-8- azabicyclo[3.2]octane-3-carboxamide |
| 724 | 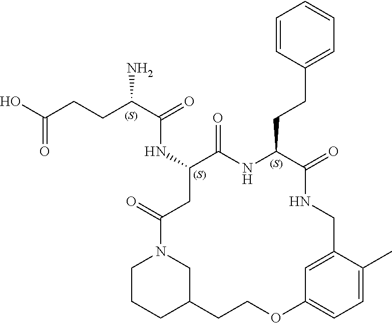 | (4S)-4-amino-5-(((9S,12S)-5⁴-methyl-8,11,14- trioxo-9-phenethyl-4-oxa-7,10-diaza-1(3,1)- piperidina-5(1,3)- benzenacyclotetradecaphane-12-yl)amino)-5- oxopentanoic acid |
| 725 | 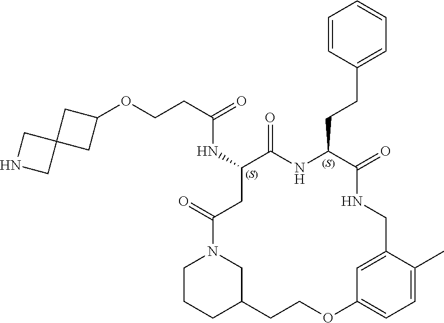 | 3-((2-azaspiro[3.3]heptan-6-yl)oxy)-N- ((9S,12S)-5⁴-methyl-8,11,14-trioxo-9- phenethyl-4-oxa-7,10-diaza-1(3,1)-piperidina- 5(1,3)-benzenacyclotetradecaphane-12- yl)propanamide |
| 726 | 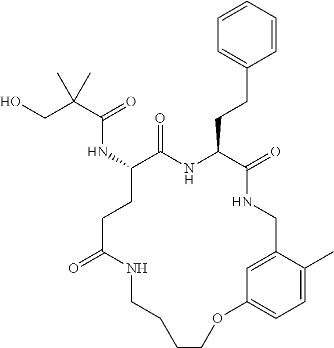 [SMILES: Cc1ccc2cc1CNC(=O)[C@H](CCc1ccccc1)NC(=O)[C@@H](NC(=O)C(C)(C)CO)CCC(=O)NCCCCO2] | 3-hydroxy-2,2-dimethyl-N-((11S,14S)-1⁴_ methyl-8,12,15-trioxo-1⁴-phenethyl-2-oxa- 7,13,16-triaza-1(1,3)- benzenacycloheptadecaphane-11- yl)propanamide |
| 727 | 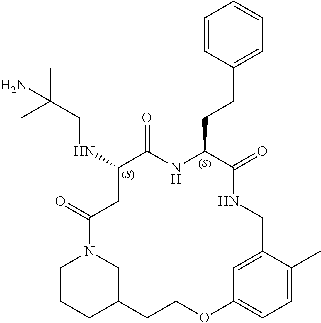 | (9S,12S)-12-((2-amino-2- methylpropyl)amino)-5⁴-methyl-9-phenethyl- 4-oxa-7,10-diaza-1(3,1)-piperidina-5(1,3)- benzenacyclotetradecaphane-8,11,14-trione |
| 728 | 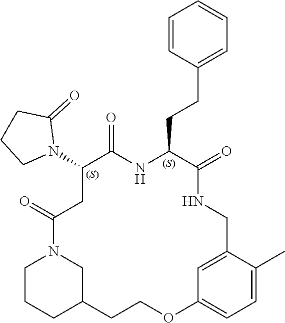 | (9S,12S)-5⁴-methyl-12-(2-oxopyrrolidin-1-yl)- 9-phenethyl-4-oxa-7,10-diaza-1(3,1)- piperidina-5(1,3)- benzenacyclotetradecaphane-8,11,14-trione |
| 729 | 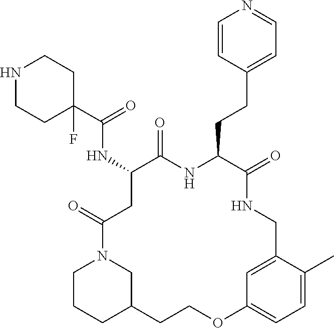 [SMILES: Cc1ccc2cc1CNC(=O)[C@H](CCc1ccncc1)NC(=O)[C@@H](NC(=O)C1(F)CCNCC1)CC(=O)N1CCCC(CCO2)C1] | 4-fluoro-N-((12S)-5⁴-methyl-8,11,14-trioxo-9- (2-(pyridin-4-yl)ethyl)-4-oxa-7,10-diaza- 1(3,1)-piperidina-5(1,3)- benzenacyclotetradecaphane-12- yl)piperidine-4-carboxamide |
| 730 | 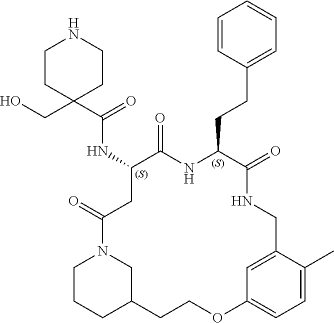 | 4-(hydroxymethyl)-N-((9S,12S)-5⁴-methyl- 8,11,14-trioxo-9-phenethyl-4-oxa-7,10-diaza- 1(3,1)-piperidina-5(1,3)- benzenacyclotetradecaphane-12- yl)piperidine-4-carboxamide |
| 731 | 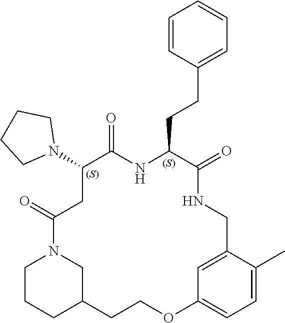 | (9S,12S)-5⁴-methyl-9-phenethyl-12- (pyrrolidin-1-yl)-4-oxa-7,10-diaza-1(3,1)- piperidina-5(1,3)- benzenacyclotetradecaphane-8,11,14-trione |
| 732 | 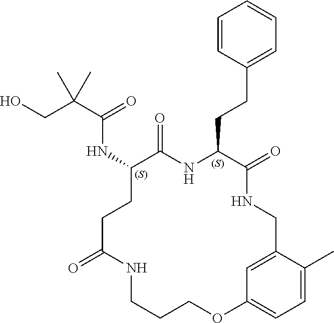 | 3-hydroxy-2,2-dimethyl-N-((10S,13S)-1⁴- methyl-7,11,14-trioxo-13-phenethyl-2-oxa- 6,12,15-triaza-1(1,3)- benzenacyclohexadecaphane-10- yl)propanamide |
| 733 | 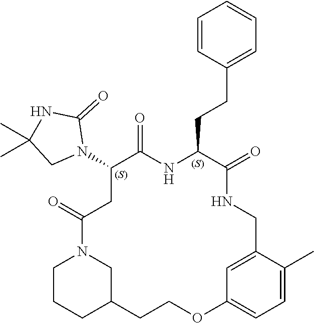 | (9S,12S)-12-(4,4-dimethyl-2-oxoimidazolidin- 1-yl)-5⁴-methyl-9-phenethyl-4-oxa-7,10-diaza- 1(3,1)-piperidina-5(1,3)- benzenacyclotetradecaphane-8,11,14-trione |
| 734 | 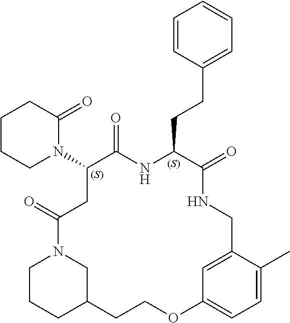 | (9S,12S)-5⁴-methyl-12-(2-oxopiperidin-1-yl)- 9-phenethyl-4-oxa-7,10-diaza-1(3,1)- piperidina-5(1,3)- benzenacyclotetradecaphane-8,11,14-trione |
| 735 | 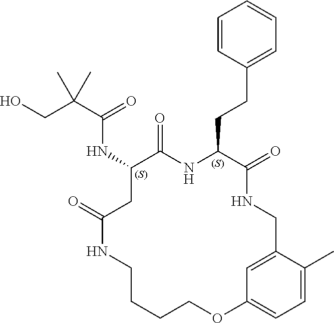 | 3-hydroxy-2,2-dimethyl-N-((10S,13S)-1⁴- methyl-8,11,14-trioxo-13-phenethyl-2-oxa- 7,12,15-triaza-1(1,3)- benzenacyclohexadecaphane-10- yl)propanamide |
| 736 | 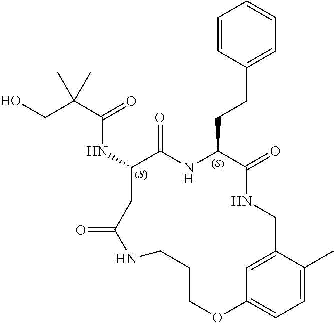 | 3-hydroxy-2,2-dimethyl-N-((9S,12S)-1⁴_ methyl-7,10,13-trioxo-12-phenethyl-2-oxa- 6,11,14-triaza-1(1,3)- benzenacyclopentadecaphane-9- yl)propanamide |
| 737 | 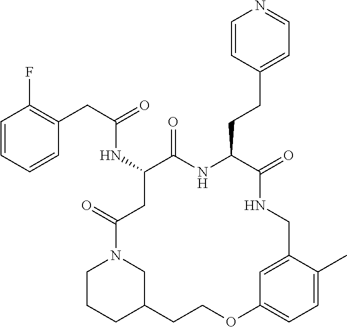 [SMILES: Cc1ccc2cc1CNC(=O)[C@H](CCc1ccncc1)NC(=O)[C@@H](NC(=O)Cc1ccccc1F)CC(=O)N1CCCC(CCO2)C1] | 2-(2-fluorophenyl)-N-((9S,12S)-5⁴-methyl- 8,11,14-trioxo-9-(2-(pyridin-4-yl)ethyl)-4-oxa- 7,10-diaza-1(3,1)-piperidina-5(1,3)- benzenacyclotetradecaphane-12- yl)acetamide |
| 738 | 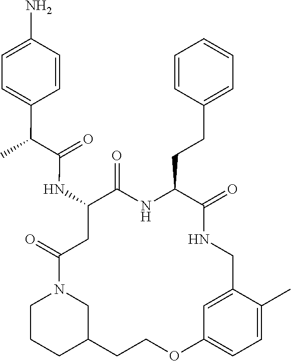 [SMILES: Cc1ccc2cc1CNC(=O)[C@H](CCc1ccccc1)NC(=O)[C@@H](NC(=O)[C@H](C)c1ccc(N)cc1)CC(=O)N1CCCC(CCO2)C1] | (2R)-2-(4-aminophenyl)-N-((9S,12S)-5⁴- methyl-8,11,14-trioxo-9-phenethyl-4-oxa- 7,10-diaza-1(3,1)-piperidina-5(1,3)- benzenacyclotetradecaphane-12- yl)propanamide |
| 739 | 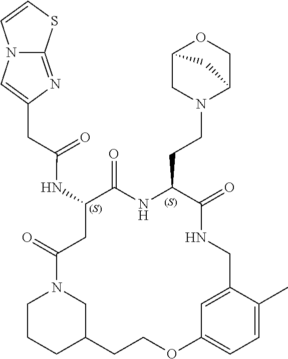 | N-((9S,12S)-9-(2-((1S,4S)-2-oxa-5- azabicyclo[2.2.1]heptan-5-yl)ethyl)-5⁴-methyl- 8,11,14-trioxo-4-oxa-7,10-diaza-1(3,1)- piperidina-5(1,3)- benzenacyclotetradecaphane-12-yl)-2- (imidazo[2,1-b]thiazol-6-yl)acetamide |
| 740 | 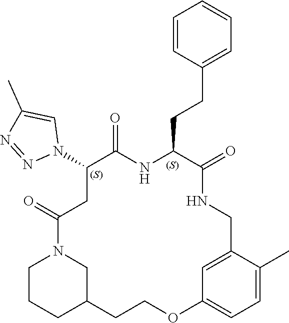 | (9S,12S)-5⁴-methyl-12-(4-methyl-1H-1,2,3- triazol-1-yl)-9-phenethyl-4-oxa-7,10-diaza- 1(3,1)-piperidina-5(1,3)- benzenacyclotetradecaphane-8,11,14-trione |
| 741 | 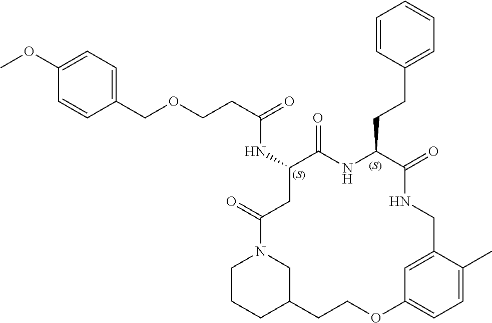 | 3-((4-methoxybenzyl)oxy)-N-((9S,12S)-5⁴- methyl-8,11,14-trioxo-9-phenethyl-4-oxa- 7,10-diaza-1(3,1)-piperidina-5(1,3)- benzenacyclotetradecaphane-12- yl)propanamide |

or an enantiomer, a stereoisomeric form, a mixture of enantiomers, a diastereomer, a mixture of diastereomers, a hydrate, a solvate, an acid salt form, a tautomer, a racemate of the above mentioned compounds, or a pharmaceutically acceptable salts thereof.

### Syntheses of Compounds

The compound of formula (I) is prepared by reference to the methods illustrated in the following schemes 1-3 by suitable selection of reagents with appropriate substitutions. Solvents, temperatures, pressures, and other reaction conditions may readily be selected by one of ordinary skill in the art. Starting materials are commercially available or readily prepared by one of ordinary skill in the art.

Another aspect of the present invention is directed to a method for producing the compound of the formula (I) comprising:
Step 1A: providing an intermediate compound (I-1*):

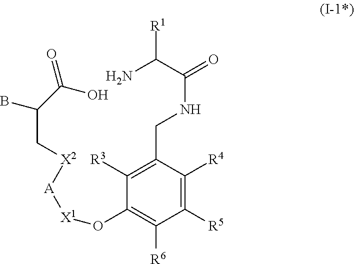

wherein
A, B, R¹, R³, R⁴, R⁵, R⁶, X¹, and X² have the same meanings as defined in the formula (I);
Step 2A: performing an intramolecular amide coupling reaction between a carboxylic acid group and an amine group of the intermediate compound (I-1*) to obtain the compound of the formula (I)

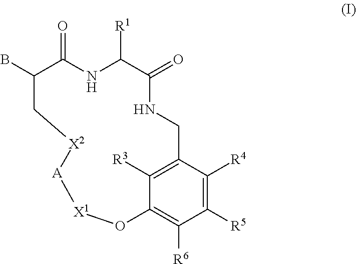

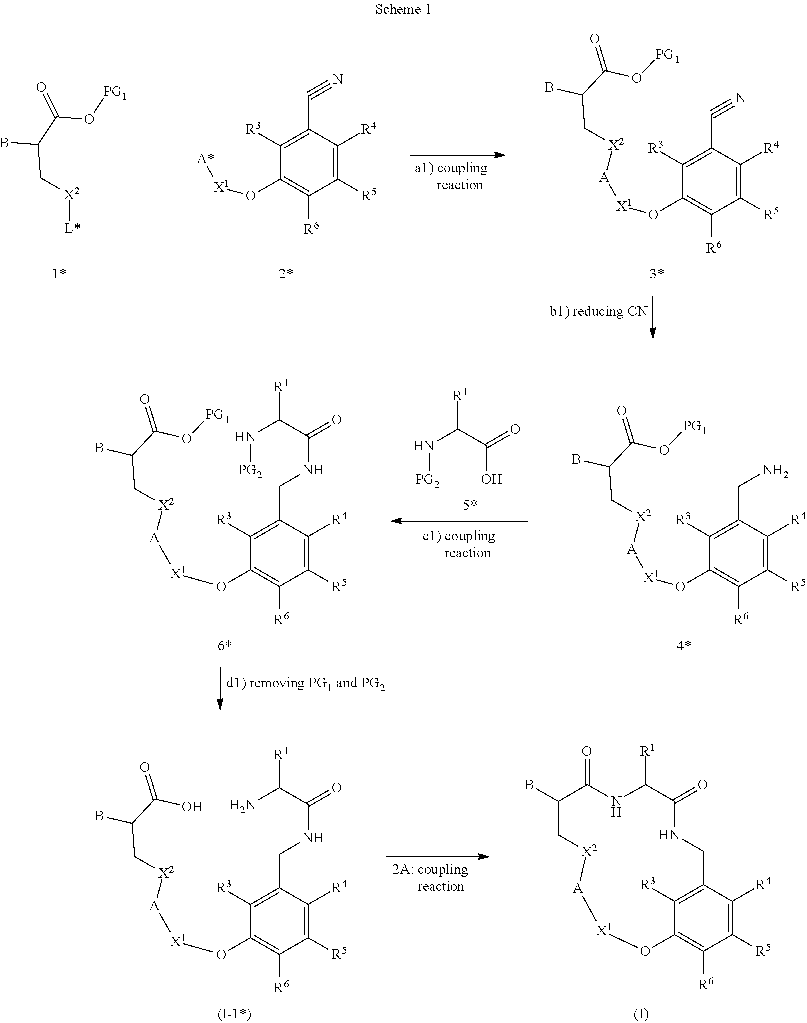

Optionally, the intermediate compound (I-1*) of Step 1A is prepared by Step 1A′.

Step1A′ comprises the following steps a1) to d1):
a1) performing a coupling reaction between a compound 1* and a compound 2*

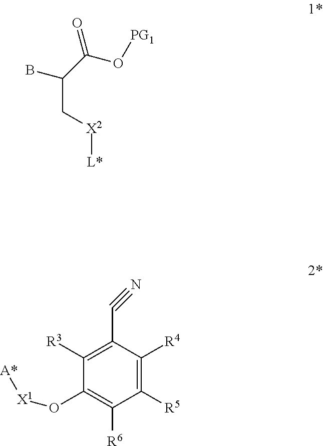

to obtain a compound 3*

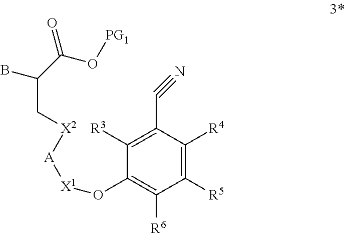

b1) reducing a nitril (—CN) group of the compound 3* to an aminomethyl (—CH₂NH₂) group to obtain a compound 4*

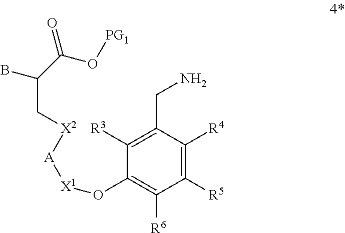

c1) performing a coupling reaction between the compound 4* and a compound 5*

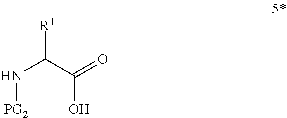

to obtain a compound 6*

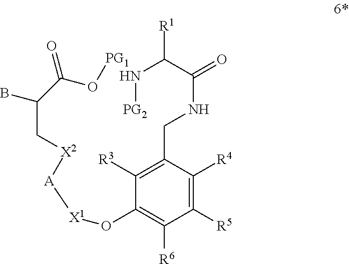

d1) removing a carboxyl protecting group PG₁ and an amine protecting group PG₂ to obtain a compound (I-1*)

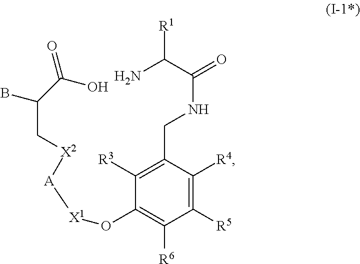

wherein
A* represents —NH(R^{N6})—,

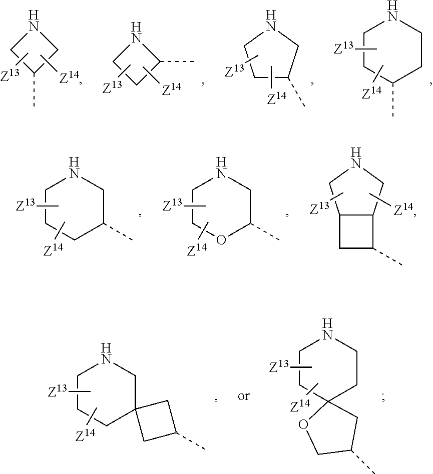

L* represents —CO₂H;
PG₁ represents a carboxyl protecting group;
PG₂ represents an amine protecting group;
and A, B, R¹, R³, R⁴, R⁵, R⁶, R^{N6}, X¹, X², Z¹³, and Z¹⁴ have the same meanings as defined in the formula (I).

Thus, the method for producing the compound of the formula (I) may comprise Step 1A′, Step 1A, and Step 2A.

Alternatively, the compound of the formula (I) is produced by the following method and thus the present invention refers to a method for producing the compound of the formula (I) comprising:
Step 1B: providing an intermediate compound (I-2*):

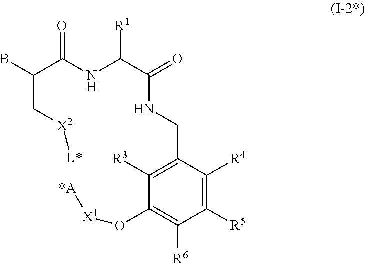

wherein
A* represents —NH(R^{N6})—,

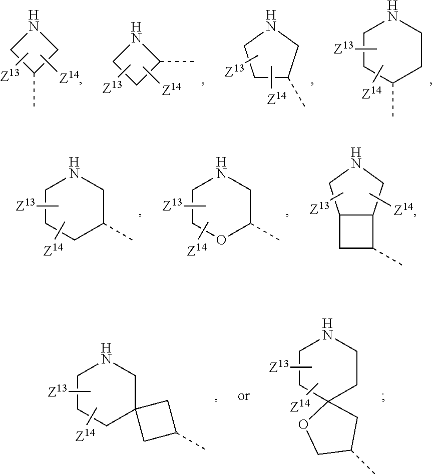

L* represents —CO₂H,
and B, R¹, R³, R⁴, R⁵, R⁶, R^{N6}, X¹, X², Z¹³, and Z¹⁴ have the same meanings as defined in the formula (I);
Step 2B: perform an intramolecular amide coupling reaction between the L* and an amino group of A* moiety of the intermediated compound (I-2*) to obtain the compound of the formula (I).

Optionally, the intermediate compound (I-2*) of Step 1B is prepared by Step 1B′.
Step1B′ comprises the following steps a2) to c2):
a2) performing a coupling reaction between a compound 5* and a compound 7*

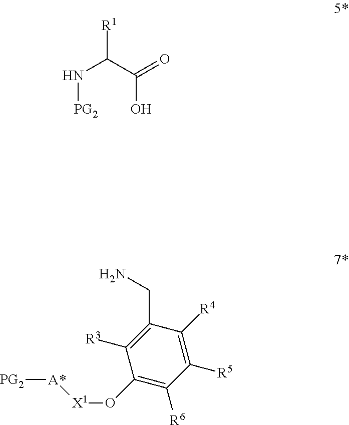

to obtain a compound 8*

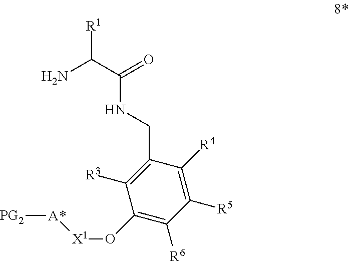

b2) preforming a coupling reaction between the compound 8* and a compound 1a*

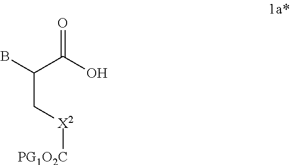

to obtain a compound 12*

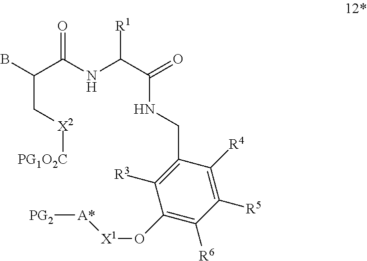

c2) removing a carboxyl protecting group PG₁ and an amine protecting group PG₂ of the compound 12* to obtain a compound (I-2*)

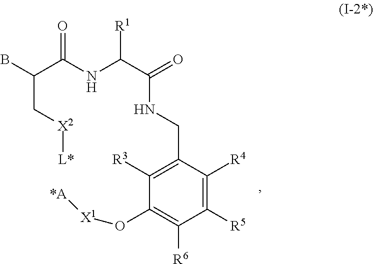

wherein
A* represents —NH(R^{N6})—,

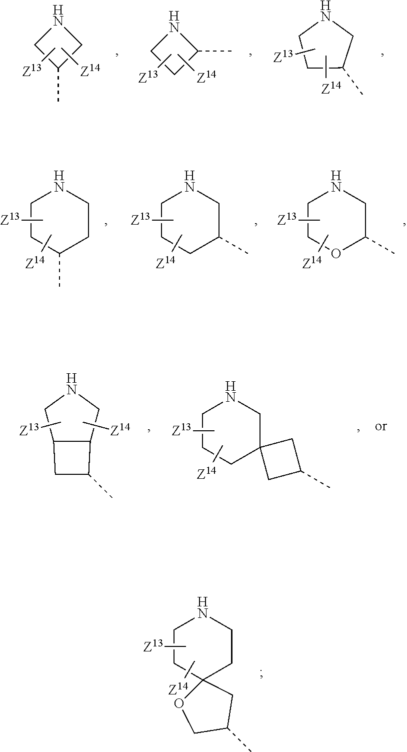

L* represents —CO₂H,
PG₁ represents a carboxyl protecting group;
PG₂ represents an amine protecting group;
and B, R¹, R², R³, R⁴, R⁵, R⁶, R^{N6}, X¹, X², Z¹³, and Z¹⁴ have the same meanings as defined in the formula (I).

Thus, the method for producing the compound of the formula (I) may comprise Step 1B′, Step 1B, and Step 2B.

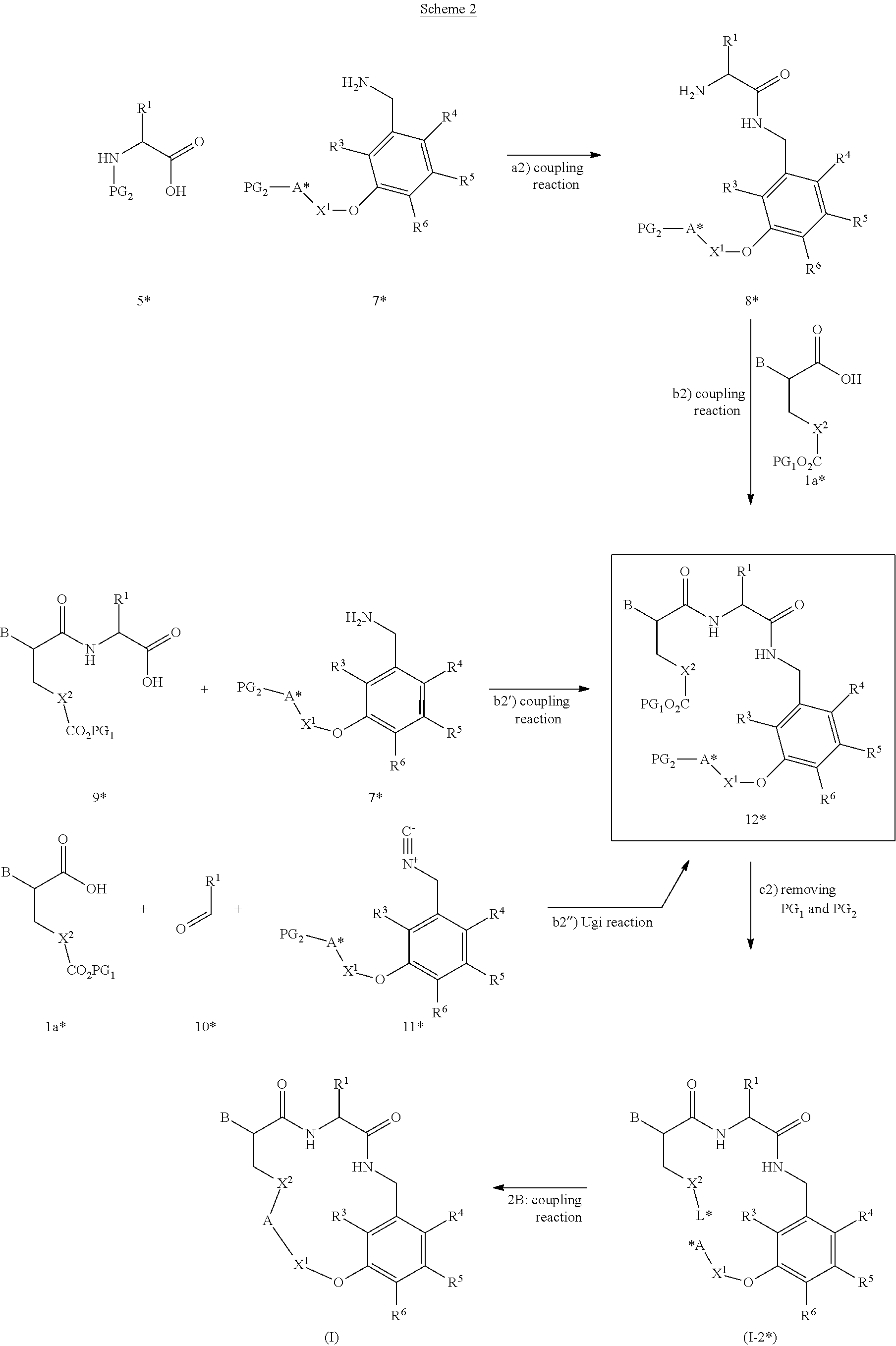

Alternatively, the intermediate compound (I-2*) of Step 1B is prepared by Step 1B″.

Step1B″ comprises the following steps b2′) and c2):
b2′) performing a coupling reaction between the compound 7* and a compound 9*

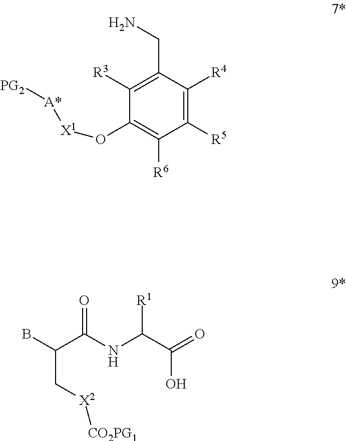

to obtain a compound 12*

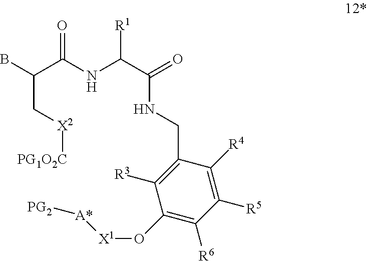

c2) removing a carboxyl protecting group PG₁ and an amine protecting group PG₂ of the compound 12* to obtain a compound (I-2*)

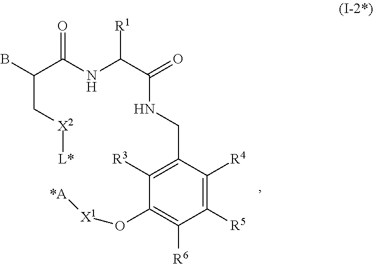

wherein
A* represents —NH(R^{N6})—,

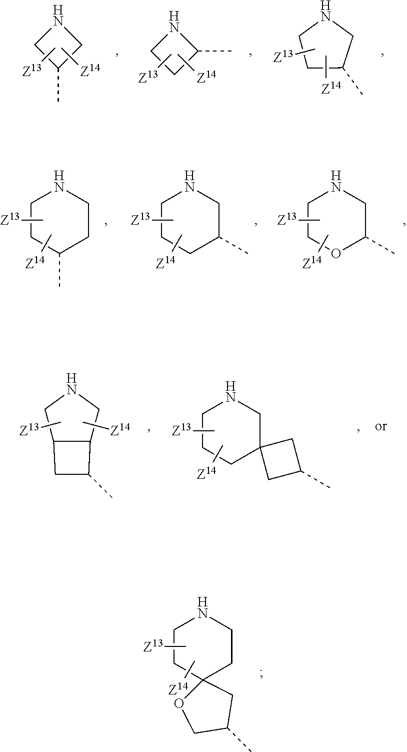

L* represents —CO₂H,
PG₁ represents a carboxyl protecting group;
PG₂ represents an amine protecting group;
and B, R¹, R², R³, R⁴, R⁵, R⁶, R^{N6}, X¹, X², Z¹³, and Z¹⁴ have the same meanings as defined in the formula (I).

Thus, the method for producing the compound of the formula (I) may comprise Step 1B′″, Step 1B, and Step 2B.

Alternatively, the intermediate compound (I-2*) of Step 1B is prepared by Step 1B′″. Step1B′″ comprises the following steps b2″) and c2):
b2″) performing a Ugi reaction of a compound 1a*, a compound 10a* (R¹—CHO), a compound 11* and aqueous ammonia (NH₃)

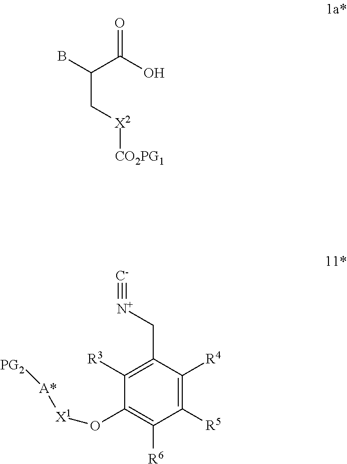

to obtain a compound 12*

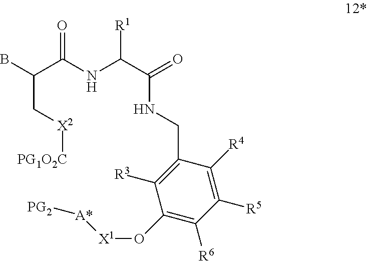

c2) removing a carboxyl protecting group PG₁ and an amine protecting group PG₂ of the compound 12* to obtain a compound (I-2*)

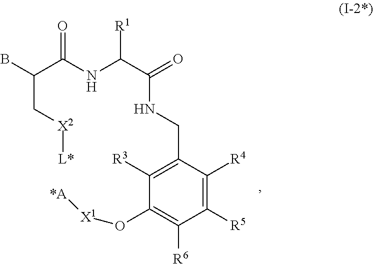

wherein
A* represents —NH(R^{N6})—,

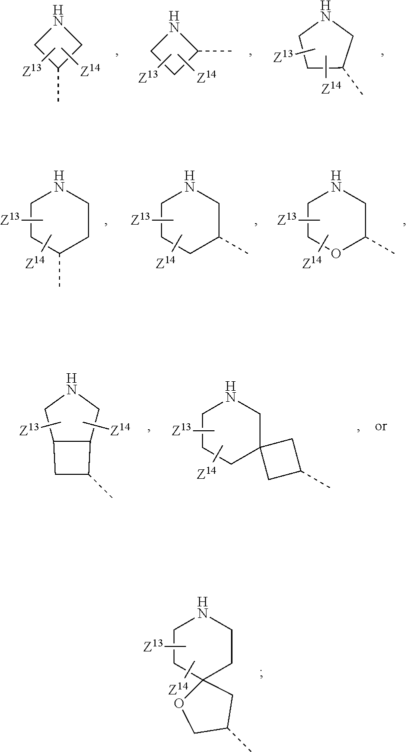

L* represents —CO₂H,
PG₁ represents a carboxyl protecting group;
PG₂ represents an amine protecting group;
and B, R¹, R², R³, R⁴, R⁵, R⁶, R^{N6}, X¹, X², Z¹³, and Z¹⁴ have the same meanings as defined in the formula (I).

Thus, the method for producing the compound of the formula (I) may comprise Step 1B′″, Step 1B, and Step 2B.

Preferably, the method for producing the compound of the formula (I) may comprise any one of Steps 1B′, 1B″, and 1B′″:
Step 1B′, comprising steps a2), b2), and c2);
Step 1B″, comprising steps b2′) and c2);
Step 1B′″, comprising steps b2″) and c2);

### Step 1B, and

### Step 2B.

Alternatively, the compound of the formula (I) is produced by the following method and thus the present invention refers to a method for producing the compound of the formula (I) comprising:
Step 1C: providing an intermediate compound (I-3*):

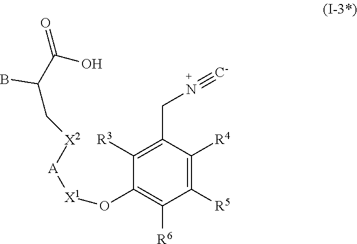

wherein
A, B, R¹, R³, R⁴, R⁵, R⁶, X¹, and X² have the same meanings as defined in the formula (I);
Step 2C: perform an intramolecular Ugi reaction of the intermediate compound (I-3*) with R¹—CHO 10* and aqueous ammonia (NH₃) to obtain the compound of the formula (I).

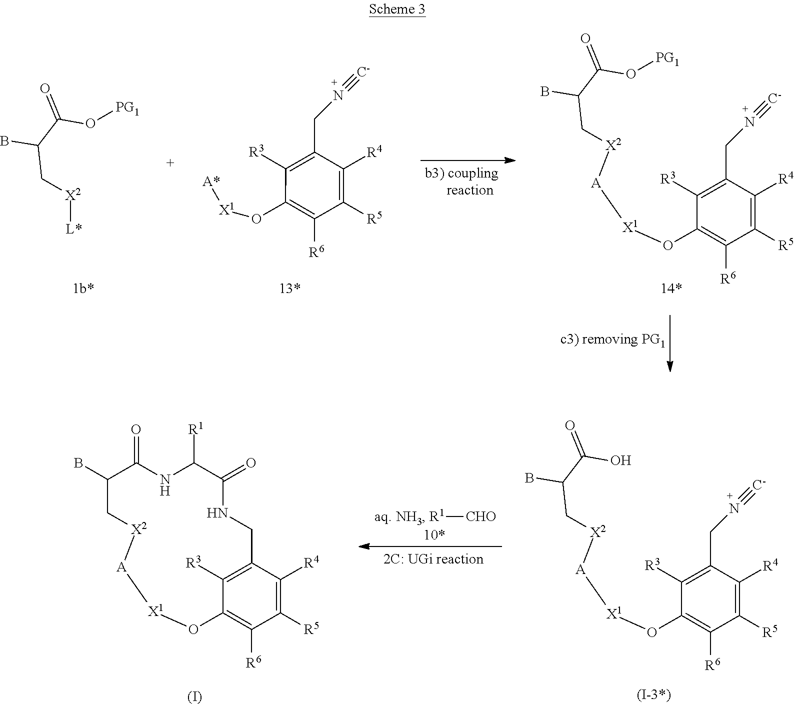

Optionally, the intermediate compound (I-3*) of Step 1C is prepared by Step 1C′.

Step1C′ comprises the following steps b3) and c3):
b3) performing a coupling reaction between the compound 1b* and a compound 13*

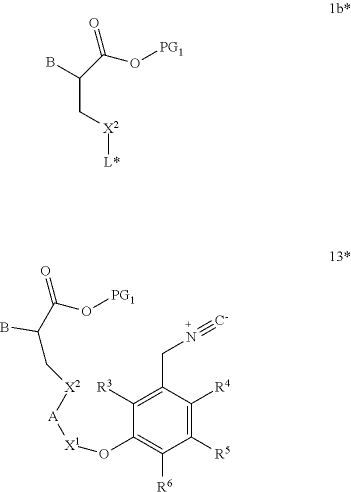

to obtain a compound 14*

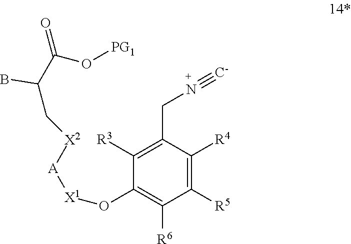

c3) removing a carboxyl protecting group PG₁ of the compound 14* to obtain a compound (I-3*)

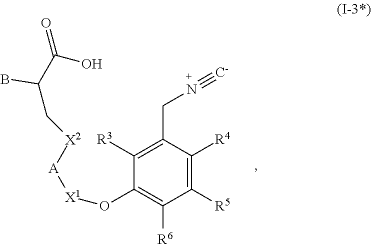

wherein
A* represents —NH(R^{N6})—,

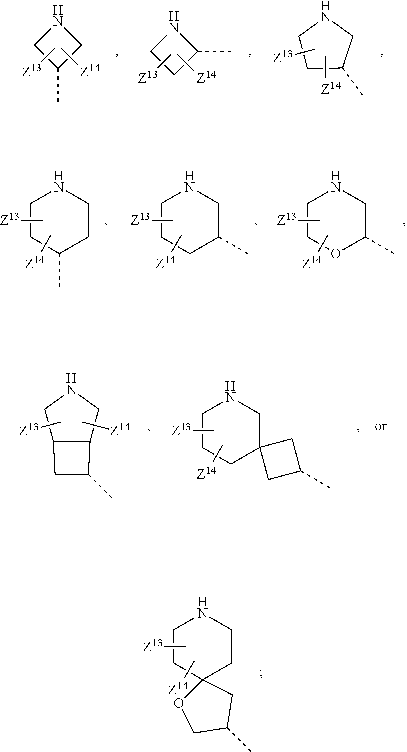

L* represents —CO₂H,
PG₁ represents a carboxyl protecting group;
and B, R¹, R³, R⁴, R⁵, R⁶, R^{N6}, X¹, X², Z¹³, and Z¹⁴ have the same meanings as defined in the formula (I).
Thus, the method for producing the compound of the formula (I) may comprise Step 1C′, Step 1C, and Step 2C.

In Steps 2A, and 2B to promote the intramolecular amide coupling reaction between a carboxylic acid group and an amino group of intermediate compound (I-1*) or (I-2*), activating reagents are commonly used for activating carboxylic acid. The activation may be introduced separate reaction or in situ reaction. Preferably, any of the following coupling reagent can be used to activate carobxylic acid group: BOP (Benzotriazole-1-yl-oxy-tris-(dimethylamino)-phosphonium hexafluorophosphate), PyBOP (Benzotriazole-1-yl-oxy-tris-pyrrolidino-phosphonium hexafluorophosphate), AOP (7-(Azabenzotriazol-1-yl)oxy tris(dimethylamino)phosphonium hexafluorophosphate), PyAOP ((7-Azabenzotriazol-1-yloxy)trispyrrolidinophosphonium hexafluorophosphate), TBTU (2-(1H-Benzotriazole-1-yl)-1,1,3,3-tetramethylaminium tetrafluoroborate), EEDQ (N-Ethoxycarbonyl-2-ethoxy-1,2-dihydroquinoline), Polyphosphoric Acid (PPA), DPPA (Diphenyl phosphoryl azide), HATU (N-[(Dimethylamino)-1H-1,2,3-triazolo-[4,5-b]pyridin-1-ylmethylene]-N-methyl methanaminium hexafluorophosphate N-oxide), HBTU (N,N,N′,N′-Tetramethyl-O-(1H-benzotriazol-1-yl)uronium hexafluorophosphate), HOBt (1-Hydroxybenzotriazole), HOAt (1-Hydroxy-7-azabenzotriazole), DCC (N,N-Dicyclohexylcarbodiimide), EDCl (N-Ethyl-N-(3-dimethylaminopropyl)carbodiimide), BOP—Cl (Bis(2-oxo-3-oxazolidinyl)phosphinic chloride), TFFH (Tetramethylfluoroformamidinium hexafluorophosphate), Brop (Bromo tris(dimethylamino) phosphonium hexafluorophosphate), PyBrop (Bromo-tris-pyrrolidino-phosphonium hexafluorophosphate) and CIP (2-Chloro-1,3-dimethylimidazolidinium hexafluorophosphate) or a mixture thereof.

Preferably, the intramolecular amide coupling reaction between a carboxylic acid group and an amino group of intermediate compound (I-1*) or (I-2*) is performed in the presence of HATU as a coupling reagent and DIPEA as a base.

Another aspect of the present invention is directed to the intermediate compounds 7*, 8*, 11*, 12*, 13*, 14*, I-1*, I-2*, and I-3*:

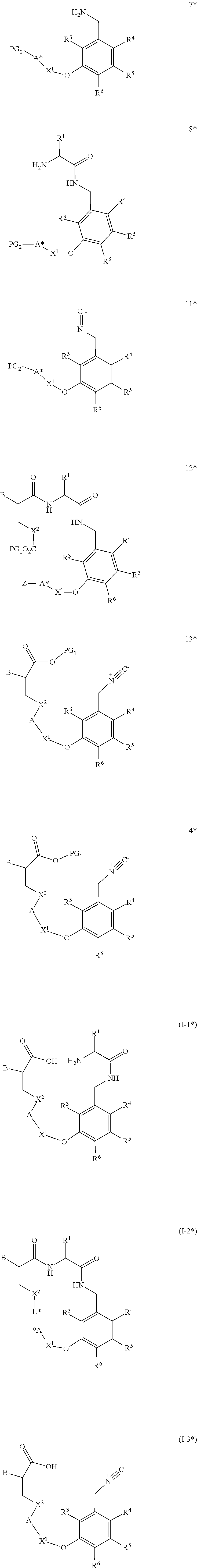

wherein
A* represents —NH(R^{N6})—,

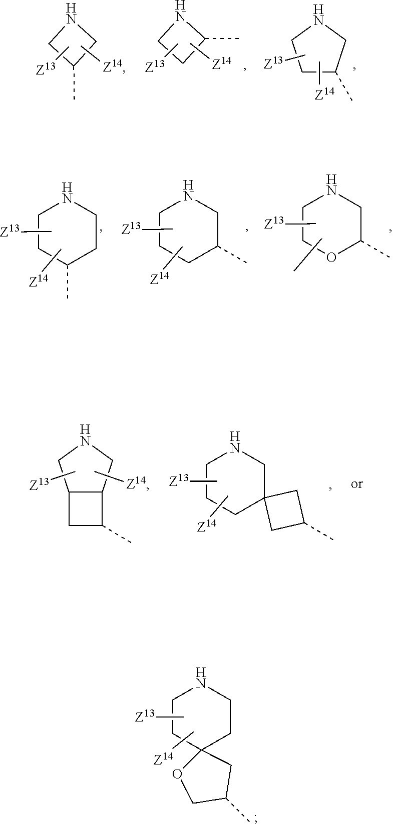

L* represents —CO₂H,
PG₁ represents a carboxyl protecting group;
PG₂ represents an amine protecting group;
and B, R¹, R², R³, R⁴, R⁵, R⁶, R^{N6}, X¹, X², Z¹³, and Z¹⁴ have the same meanings as defined in the formula (I).

### Indication

In a further aspect of the present invention, the novel compounds according to the general formula (I) are used as pharmaceutically active agent.

Surprisingly it was found that the above-mentioned compounds of general formula (I), as well as the pharmaceutical compositions thereof are acting as proteasome inhibitors, and more specifically as inhibitors of proteasome subunit beta type-5.

In the present application, the inhibitory activity assays were performed to determine IC₅₀ values of compounds of general formula (I) against proteasome subunit beta type-5.

Table 2 shows activity data (indicated as IC₅₀ values) in the biochemical assays. It is proven that the inventive compounds inhibit proteasome subunit beta type-5 of both the constitutive and immunoproteasome very effectively. In addition it is shown that the inventive compounds also inhibit the subunit beta type-5 of both the constitutive and immunoproteasome in the cell based proteasome glo assays very effectively. Finally it is shown that proteasome inhibition of these compounds translates into inhibition of proliferation of tumor cells (HT29) very effectively.

Tables 3-5 show that the inventive compounds effectively inhibit proliferation of other cancer cells such as A549 (human lung carcinoma cell line), A2780 (human ovarian cancer cell line), MDA-MB-468 (breast carcinoma), Hs 746T (human gastric carcinoma cell line), MM1S (B lymphoblast cell line), and RPMI 8226 (B lymphocyte cell line, multiple myeloma).

The pharmaceutical compositions according to the present invention comprise at least one compound according to the present invention as an active ingredient together with at least one pharmaceutically acceptable (i.e. non-toxic) carrier, excipient and/or diluent.

As used herein, proteasome “inhibitor”, especially proteasome subunit beta type-5 “inhibitor” refers to any compound capable of downregulating, decreasing, suppressing or otherwise regulating the amount and/or activity of a proteasome can be achieved by any of a variety of mechanisms known in the art, including, but not limited to binding directly to said proteasome subunit beta type-5.

As used herein the term “inhibiting” or “inhibition” refers to the ability of a compound to downregulate, decrease, reduce, suppress, inactivate, or inhibit at least partially the activity of an enzyme, or the expression of an enzyme or protein.

Proteasome subunit beta type-5 is a protein that in humans is encoded by the PSMB5 gene in case of the constitutive proteasome and by LMP7 in case of the immunoproteasome. Proteasome subunit beta type-5, along with other beta subunits, assembles into two heptameric rings and subsequently a proteolytic chamber for substrate degradation. This protein contains “chymotrypsin-like” activity and is capable of cleaving after large hydrophobic residues of peptide. The eukaryotic proteasome recognized degradable proteins, including damaged proteins for protein quality control purpose or key regulatory protein components for dynamic biological processes. An essential function of a modified proteasome, the immunoproteasome, is the processing of class I MHC peptides.

Further aspects of the present invention relate to the compound of the general formula (I), or the above-mentioned pharmaceutical composition, for use in prophylaxis and/or treatment of diseases caused by or associated with proteasome, or immunoproteasome, in particular, proteasome subunit beta type-5, selected from a cancer, an infectious disease, an inflammatory disease, autoimmune disease and transplant rejection.

Further aspect of the present invention relates to the use of the compound of general formula (I) for the preparation of a pharmaceutical composition useful for prophylaxis and/or treatment of diseases caused by or associated with proteasome, or immunoproteasome, in particular, proteasome subunit beta type-5, selected from a cancer, an infectious disease, an inflammatory disease, autoimmune disease and transplant rejection.

In a further aspect of the present invention, methods for preventing and/or treating diseases caused by or associated with proteasome, or immunoproteasome, in particular, proteasome subunit beta type-5 in a mammal, especially in a human, are provided, which methods comprise administering to the mammal an amount of at least one compound according to the general formula (I) and/or pharmaceutically acceptable salts thereof, effective to prevent and/or treat the diseases caused by or associated with proteasome, or immunoproteasome, in particular, proteasome subunit beta type-5 selected from a cancer, a neurodegenerative disease, an infectious disease, an inflammatory disease, autoimmune disease and to suppress allograft rejection during transplantation.

The term “effective amount” means an amount of compound that, when administered to a patient in need of such treatment, is sufficient to
(i) treat or prevent a particular disease, condition, or disorder which can be treated with a proteasome inhibitor;
(ii) attenuate, ameliorate, or eliminate one or more symptoms of the particular disease, condition, or disorder; or
(iii) prevent or delay the onset of one or more symptoms of the particular disease, condition, or disorder described herein.

The amount of a compound of general formula (I) that will correspond to such an amount will vary depending upon factors such as the particular compound, disease condition and its severity, the identity (e.g., weight) of the patient in need of treatment, but can nevertheless be routinely determined by one skilled in the art.

### Cancer

The compounds of the present application or the pharmaceutical composition thereof are useful for the treatment and/or prophylaxis of cancer, wherein the cancer is selected from the group consisting of: adenocarcinoma, choroidal melanoma, acute leukemia, acoustic neurinoma, ampullary carcinoma, anal carcinoma, astrocytoma, basal cell carcinoma, pancreatic cancer, desmoid tumour, bladder cancer, bronchial carcinoma, non-small cell lung cancer (NSCLC), breast cancer, Burkitt's lymphoma, corpus cancer, CUP-syndrome (carcinoma of unknown primary), colorectal cancer, small intestine cancer, small intestinal tumours, ovarian cancer, endometrial carcinoma, ependymoma, epithelial cancer types, Ewing's tumours, gastrointestinal tumours, gastric cancer, gallbladder cancer, gall bladder carcinomas, uterine cancer, cervical cancer, cervix, glioblastomas, gynecologic tumours, ear, nose and throat tumours, hematologic neoplasias, hairy cell leukemia, urethral cancer, skin cancer, skin testis cancer, brain tumours (gliomas), brain metastases, testicle cancer, hypophysis tumour, carcinoids, Kaposi's sarcoma, laryngeal cancer, germ cell tumour, bone cancer, colorectal carcinoma, head and neck tumours (tumours of the ear, nose and throat area), colon carcinoma, craniopharyngiomas, oral cancer (cancer in the mouth area and on lips), cancer of the central nervous system, liver cancer, liver metastases, leukemia, eyelid tumor, lung cancer, lymph node cancer (Hodgkin's/Non-Hodgkin's), lymphomas, stomach cancer, malignant melanoma, malignant neoplasia, malignant tumours gastrointestinal tract, breast carcinoma, rectal cancer, medulloblastomas, melanoma, meningiomas, Hodgkin's disease, mycosis fungoides, nasal cancer, neurinoma, neuroblastoma, kidney cancer, renal cell carcinomas, non-Hodgkin's lymphomas, oligodendroglioma, esophageal carcinoma, osteolytic carcinomas and osteoplastic carcinomas, osteosarcomas, ovarial carcinoma, pancreatic carcinoma, penile cancer, plasmocytoma, squamous cell carcinoma of the head and neck (SCCHN), prostate cancer, pharyngeal cancer, rectal carcinoma, retinoblastoma, vaginal cancer, thyroid carcinoma, Schneeberger disease, esophageal cancer, spinalioms, T-cell lymphoma (mycosis fungoides), thymoma, tube carcinoma, eye tumours, urethral cancer, urologic tumours, urothelial carcinoma, vulva cancer, wart appearance, soft tissue tumours, soft tissue sarcoma, Wilm's tumour, cervical carcinoma, tongue cancer, astrocytomas, bronchial cancer, laryngeal cancer, malignant melanoma, oesophageal cancer, cholangiocarcinoma, and renal cell cancer.

Preferrably, the present application or the pharmaceutical composition thereof are useful for the treatment and/or prophylaxis of cancer, wherein the cancer is leukemia, multiple myeloma, mantle-cell lymphoma (MCL), breast cancer, colorectal cancer, non-small cell lung cancer or ovarian cancer, more preferably, the cancer is multiple myeloma.

Optionally, the compound of the present invention, or the pharmaceutical composition thereof is used for treatment/prophylaxis of said cancer, in particular, multiple myeloma in combination with a thalidomide or a derivative thereof.

The derivative of thalidomide is selected from lenalidomide, pomalidomide, avadomide, and iberdomide and CC-885. CC-885 is a modulator of the E3 ligase protein cereblon with antiproliferative effects on human myeloid leukemia cell lines. It forms a complex with cereblon and the cell cycle regulator and translation termination factor GSPT1. The antitumour activity of CC-885 relies on cereblon-dependent ubiquitination and degradation of the GSPT1. Formal Name: N-(3-chloro-4-methylphenyl)-N′—[[2-(2,6-dioxo-3-piperidinyl)-2,3-dihydro-1-oxo-1H-isoindol-5-yl]methyl]-urea (CAS Number: 1010100-07-8).

### Infectious Disease

The compounds of the present application or the pharmaceutical composition thereof are useful for the treatment and/or prophylaxis of the infectious disease, wherein the infectious disease is selected from the group consisting of: HIV, Echinococcosis, Amebiasis (Entamoeba histolytica Infection), Angiostrongylus Infection, Anisakiasis, Anthrax, Babesiosis (Babesia Infection), Balantidium Infection (Balantidiasis), Baylisascaris Infection (Raccoon Roundworm), Bilharzia (Schistosomiasis), Blastocystis hominis Infection (Blastomycosis), Borreliosis, Botulism, Brainerd Diarrhea, Brucellosis, BSE (Bovine Spongiform Encephalopathy), Candidiasis, Capillariasis (Capillaria Infection), CFS (Chronic Fatigue Syndrome), Chagas Disease (American Trypanosomiasis), Chickenpox (Varicella-Zoster virus), Chlamydia pneumoniae Infection, Cholera, CJD (Creutzfeldt-Jakob Disease), Clonorchiasis (Clonorchis Infection), CLM (Cutaneous Larva Migrans, Hookworm Infection), Coccidioidomycosis, Conjunctivitis, Coxsackievirus A16 (Hand, Foot and Mouth Disease), Cryptococcosis, Cryptosporidium Infection (Cryptosporidiosis), Culex mosquito (Vector of West Nile Virus), Cyclosporiasis (Cyclospora Infection), Cysticercosis (Neurocysticercosis), Cytomegalovirus Infection, Dengue/Dengue Fever, Dipylidium Infection (Dog and Cat Flea Tapeworm), Ebola Virus Hemorrhagic Fever, Echinococcosis (Alveolar Hydatid Disease), Encephalitis, Entamoeba coli Infection, Entamoeba dispar Infection, Entamoeba hartmanni Infection, Entamoeba histolytica Infection (Amebiasis), Entamoeba polecki Infection, Enterobiasis (Pinworm Infection), Enterovirus Infection (non-polio), Epstein-Barr Virus Infection, Escherichia coli Infection, Foodborne Infection, Foot and mouth Disease, Fungal Dermatitis, Gastroenteritis, Group A streptococcal Disease, Group B streptococcal Disease, Hansen's Disease (Leprosy), Hantavirus Pulmonary Syndrome, Head Lice Infestation (Pediculosis), Helicobacter pylori Infection, Hematologic Disease, Hendra Virus Infection, Hepatitis (HCV, HBV), Herpes Zoster (Shingles), Human Ehrlichiosis, Human Parainfluenza Virus Infection, Influenza, Isosporiasis (Isospora Infection), Lassa Fever, Leishmaniasis, Kala-azar (Kala-azar, Leishmania Infection), Leprosy, Lice (Body lice, Head lice, Pubic lice), Lyme Disease, Malaria, Marburg Hemorrhagic Fever, Measles, Meningitis, Mosquito-borne Diseases, Mycobacterium avium Complex (MAC) Infection, Naegleria Infection, Nosocomial Infections, Nonpathogenic Intestinal Amebae Infection, Onchocerciasis (River Blindness), Opisthorciasis (Opisthorcis Infection), Parvovirus Infection, Plague, PCP (Pneumocystis carinii Pneumonia), Polio, Q Fever, Rabies, Respiratory Syncytial Virus (RSV) Infection, Rheumatic Fever, Rift Valley Fever, Rotavirus Infection, Roundworms Infection, Salmonellosis, Salmonella Enteritidis, Scabies, Shigellosis, Shingles, Sleeping Sickness, Smallpox, Streptococcal Infection, Tapeworm Infection (Taenia Infection), Tetanus, Toxic Shock Syndrome, Tuberculosis, Ulcers (Peptic Ulcer Disease), Valley Fever, Vibrio parahaemolyticus Infection, Vibrio vulnificus Infection, Viral Hemorrhagic Fever, Warts, Waterborne infectious Diseases, West Nile Virus Infection (West Nile Encephalitis), Whooping Cough, Yellow Fever.

### Inflammatory Disease

Inflammatory disease is caused by cytokines, TNF-α, IL-1β, GM-CSF, IL-6/IL-8, adhesion molecules (ICAM-1, VCAM-1, P-selectin) and prostaglandins and/or nitric oxide (NO).

### Autoimmune Disease

Furthermore, the compounds of the present application or the pharmaceutical composition thereof are useful for the treatment and/or prophylaxis of autoimmune disease. The autoimmune disease is selected from the group consisting of: Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Bald disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS), Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myelin Oligodendrocyte Glycoprotein Antibody Disorder, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatic, Polymyositis, Postmyocardial infarction syndrome, Postpericardiotomy syndrome, Primary Biliary Cholangitis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Thyroid eye disease (TED), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, Vogt-Koyanagi-Harada Disease.

Preferred, the autoimmune disease is selected from Lupus nephritis, lupus, systemic lupus erythematosus, myasthenia gravis, multiple sclerosis, polyarthritis, rheumatoid arthritis, irritant sensitivity, psoriasis, asthma, and colitis, more preferred, the autoimmune disease is myasthenia gravis.

Transplant rejection occurs when transplanted tissue is rejected by the recipient's immune system, which destroys the transplanted tissue. Transplant rejection can be lessened by determining the molecular similitude between donor and recipient and by use of immunosuppressant drugs after transplant. The compound of the present invention may be used as immunosuppressant drugs.

The compounds enlisted explicitly in Table 1 are preferred to be used within the methods or indications disclosed herein.

The pharmaceutical compositions of the present invention can be prepared in a conventional solid or liquid carrier or diluent and a conventional pharmaceutically-made adjuvant at suitable dosage level in a known way. The preferred preparations are adapted for oral application. These administration forms include, for example, pills, tablets, film tablets, coated tablets, capsules, powders and deposits.

Furthermore, the present invention also includes pharmaceutical preparations for parenteral application, including dermal, intradermal, intragastral, intracutan, intravasal, intravenous, intramuscular, intraperitoneal, intranasal, intravaginal, intrabuccal, percutan, rectal, subcutaneous, sublingual, topical, or transdermal application, which preparations in addition to typical vehicles and/or diluents contain at least one compound according to the present invention and/or a pharmaceutical acceptable salt thereof as active ingredient.

The pharmaceutical compositions according to the present invention containing at least one compound according to the present invention and/or a pharmaceutical acceptable salt thereof as active ingredient will typically be administered together with suitable carrier materials selected with respect to the intended form of administration, i.e. for oral administration in the form of tablets, capsules (either solid filled, semi-solid filled or liquid filled), powders for constitution, gels, elixirs, dispersable granules, syrups, suspensions, and the like, and consistent with conventional pharmaceutical practices. For example, for oral administration in the form of tablets or capsules, the active drug component may be combined with any oral non-toxic pharmaceutically acceptable carrier, preferably with an inert carrier like lactose, starch, sucrose, cellulose, magnesium stearate, dicalcium phosphate, calcium sulfate, talc, mannitol, ethyl alcohol (liquid filled capsules) and the like. Moreover, suitable binders, lubricants, disintegrating agents and coloring agents may also be incorporated into the tablet or capsule. Powders and tablets may contain about 5 to about 95-weight % of the compounds of the formula (I) or the respective pharmaceutically active salt as active ingredient.

Suitable binders include starch, gelatin, natural sugars, corn sweeteners, natural and synthetic gums such as acacia, sodium alginate, carboxymethylcellulose, polyethylene glycol and waxes. Among suitable lubricants there may be mentioned boric acid, sodium benzoate, sodium acetate, sodium chloride, and the like. Suitable disintegrants include starch, methylcellulose, guar gum, and the like. Sweetening and flavoring agents as well as preservatives may also be included, where appropriate. The disintegrants, diluents, lubricants, binders etc. are discussed in more detail below.

Moreover, the pharmaceutical compositions of the present invention may be formulated in sustained release form to provide the rate controlled release of any one or more of the components or active ingredients to optimise the therapeutic effect(s), e.g. antihistaminic activity and the like. Suitable dosage forms for sustained release include tablets having layers of varying disintegration rates or controlled release polymeric matrices impregnated with the active components and shaped in tablet form or capsules containing such impregnated or encapsulated porous polymeric matrices.

Liquid form preparations include solutions, suspensions, and emulsions. As an example, there may be mentioned water or water/propylene glycol solutions for parenteral injections or addition of sweeteners and opacifiers for oral solutions, suspensions, and emulsions. Liquid form preparations may also include solutions for intranasal administration.

Aerosol preparations suitable for inhalation may include solutions and solids in powder form, which may be present in combination with a pharmaceutically acceptable carrier such as an inert, compressed gas, e.g. nitrogen.

For preparing suppositories, a low melting wax, such as a mixture of fatty acid glycerides like cocoa butter is melted first, and the active ingredient is then dispersed homogeneously therein e.g. by stirring. The molten, homogeneous mixture is then poured into conveniently sized moulds, allowed to cool, and thereby solidified.

Also included are solid form preparations, which are intended to be converted, shortly before use, to liquid form preparations for either oral or parenteral administration. Such liquid forms include solutions, suspensions, and emulsions.

The compounds according to the present invention may also be delivered transdermally. The transdermal compositions may have the form of a cream, a lotion, an aerosol and/or an emulsion and may be included in a transdermal patch of the matrix or reservoir type as is known in the art for this purpose.

The term capsule as recited herein refers to a specific container or enclosure made e.g. of methylcellulose, polyvinyl alcohols, or denatured gelatins or starch for holding or containing compositions comprising the active ingredient(s). Capsules with hard shells are typically made of blended of relatively high gel strength gelatins from bones or pork skin. The capsule itself may contain small amounts of dyes, opaquing agents, plasticisers and/or preservatives.

Under tablet a compressed or moulded solid dosage form is understood which comprises the active ingredients with suitable diluents. The tablet may be prepared by compression of mixtures or granulations obtained by wet granulation, dry granulation, or by compaction well known to a person of ordinary skill in the art.

Oral gels refer to the active ingredients dispersed or solubilised in a hydrophilic semi-solid matrix.

Powders for constitution refers to powder blends containing the active ingredients and suitable diluents which can be suspended e.g. in water or in juice.

Suitable diluents are substances that usually make up the major portion of the composition or dosage form. Suitable diluents include sugars such as lactose, sucrose, mannitol, and sorbitol, starches derived from wheat, corn rice, and potato, and celluloses such as microcrystalline cellulose. The amount of diluent in the composition can range from about 5 to about 95% by weight of the total composition, preferably from about 25 to about 75 weight %, and more preferably from about 30 to about 60 weight %.

The term disintegrants refers to materials added to the composition to support break apart (disintegrate) and release the pharmaceutically active ingredients of a medicament. Suitable disintegrants include starches, “cold water soluble” modified starches such as sodium carboxymethyl starch, natural and synthetic gums such as locust bean, karaya, guar, tragacanth and agar, cellulose derivatives such as methylcellulose and sodium carboxymethylcellulose, microcrystalline celluloses, and cross-linked microcrystalline celluloses such as sodium croscarmellose, alginates such as alginic acid and sodium alginate, clays such as bentonites, and effervescent mixtures. The amount of disintegrant in the composition may range from about 2 to about 20 weight % of the composition, more preferably from about 5 to ca. 10 weight %.

Binders are substances which bind or “glue” together powder particles and make them cohesive by forming granules, thus serving as the “adhesive” in the formulation. Binders add cohesive strength already available in the diluent or bulking agent. Suitable binders include sugars such as sucrose, starches derived from wheat corn rice and potato, natural gums such as acacia, gelatin and tragacanth, derivatives of seaweed such as alginic acid, sodium alginate and ammonium calcium alginate, cellulose materials such as methylcellulose, sodium carboxymethylcellulose and hydroxypropylmethylcellulose, polyvinylpyrrolidone, and inorganic compounds such as magnesium aluminum silicate.

The amount of binder in the composition may range from about 2 to about 20 weight % of the composition, preferably from about 3 to about 10 weight %, and more preferably from about 3 to about 6 weight %.

Lubricants refer to a class of substances which are added to the dosage form to enable the tablet granules etc. after being compressed to release from the mould or die by reducing friction or wear. Suitable lubricants include metallic stearates such as magnesium stearate, calcium stearate, or potassium stearate, stearic acid, high melting point waxes, and other water soluble lubricants such as sodium chloride, sodium benzoate, sodium acetate, sodium oleate, polyethylene glycols and D,L-leucine. Lubricants are usually added at the very last step before compression, since they must be present at the surface of the granules. The amount of lubricant in the composition may range from about 0.2 to about 5 weight % of the composition, preferably from about 0.5 to about 2 weight %, and more preferably from about 0.3 to about 1.5 weight % of the composition.

Glidants are materials that prevent caking of the components of the pharmaceutical composition and improve the flow characteristics of granulate so that flow is smooth and uniform. Suitable glidants include silicon dioxide and talc. The amount of glidant in the composition may range from about 0.1 to about 5 weight % of the final composition, preferably from about 0.5 to about 2 weight %.

Coloring agents are excipients that provide coloration to the composition or the dosage form. Such excipients can include food grade dyes adsorbed onto a suitable adsorbent such as clay or aluminum oxide. The amount of the coloring agent may vary from about 0.1 to about 5 weight % of the composition, preferably from about 0.1 to about 1 weight %.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

### EXAMPLES

### Preparation of Compounds:

### General Information:

All reactions involving air- or moisture-sensitive reagents or intermediates were carried out in flame-dried glassware under an argon atmosphere. Dry solvents (THF, toluene, MeOH, DMF, DCM) were used as commercially available. ¹H-NMR and ¹³C-NMR were recorded on a Bruker DRX400 (400 MHz). Multiplicities are indicated as: br s (broadened singlet), s (singlet), d (doublet), t (triplet), q (quartet), quin (quintet), m (multiplet); and coupling constants (J) are given in Hertz (Hz). HPLC—electrospray mass spectra (HPLC ES-MS) were obtained using Waters Acquity Performance Liquid Chromatography (UPLC) equipped SQ 3100 Mass detector spectrometer. Column: Acquity UPLC BEH C18 1.7 um, 2.1×50 mm. Flow: 0.5 ml/min. Eluents: A: H₂O with 0.05% formic acid and B: ACN with 0.05% TFA. All chemicals and solvents were purchased from commercial sources like Sigma-Aldrich, Fluka, TCI, Acros Organics, ABCR, Alfa Aesar, Enamine, VWR, Combi-Blocks, Apollo Scientific, Aquilla Pharmatech, Ark Pharm, D-L Chiral Chemicals, ChemBridge, Renno Tech, Accela, KeyOrganics, Pharmablock and Chem Impex. Unless otherwise noted, all commercially available compounds were used as received without further purifications.

Abbreviations used in the description of the chemistry and in the Examples that follow are: ACN or MeCN (acetonitrile); Asp (aspartic acid), br (broad); BOC (tet-butyloxycarbonyl), Cbz (benzyloxycarbonyl), CDCl₃ (deuterated chloroform); cHex (cyclohexane); CDI (1,1′-Carbonyldiimidazole), DPCP (diphenyl chlorophosphate), DBU (1,8-diazabicyclo[5.4.0]undec-7-ene), DCE (1,2-dichloroethane), DCM (dichloromethane); DIAD (diisopropyl azodicarboxylate); DIEA (N,N-Diisopropylethylamine), DIPEA (di-iso-propylethylamine); DMF (dimethylformamide); DMSO (dimethyl sulfoxide); DPPA (diphenylphosphoryl azide), EA (ethyl acetate), eq. (equivalent); EDCl (1-ethyl-3-(3-dimethylaminopropyl)carbodiimide), ES (electrospray); EtOAc (ethyl acetate); EtOH (ethanol); Glu (glutamic acid), HATU (0-(7-azabenzotriazol-1-yl)-N,N,N′,N′-tetramethyluronium hexafluorophosphate); HCl (hydrochloric acid); HOBt (Hydroxybenzotriazole), hPhe (homophenylalanine), MeOH (methanol); MS (mass spectrometry); MTBE (methyl tert-butyl ether), Mwt (molecular weight); NMM (4-methylmorpholine), NMP (N-Methyl-2-pyrrolidone), NMR (nuclear magnetic resonance); PE (petroleum ether), RP (reversed-phase); RT/r.t. (room temperature); sat. aq. (saturated aqueous); SiO₂ (silica gel); tBu (tert-butyl), T₃P (propanephosphonic acid anhydride), TBME (tert-butyl methyl ether), TFA (trifluoroacetic acid); THF (tetrahydrofurane); TIS (triisopropylsilane).

### PREPARATIVE EXAMPLES

### General Procedure A

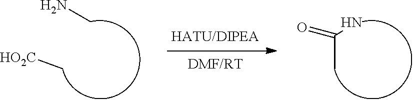

1.2 eq. HATU and 2 eq. DIPEA are dissolved in 1.5 ml/mmol DMF. 1 eq. amino acid in 2 ml/mmol DMF are added dropwise at room temperature or elevated temperature. 1 h after complete addition the reaction mixture was distributed between ethyl acetate and 2 N NaOH solution. The organic phase was separated, washed with brine, dried over sodium sulfate and solvents were evaporated. Depending on scale the raw product was purified by crystallization, flash chromatography or HPLC.

### General Procedure B

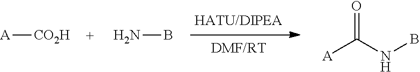

Carboxylic acid (1.5 eq.), HATU (1.1 eq.) and DIPEA (6 eq.) are dissolved in DMF and amine is added. After reaction was completed the reaction mixture can be diluted with ethyl acetate and then washed by sodium hydroxide solution and brine. After drying the organic phase over sodium sulfate and removing the solvents under reduced pressure the raw product was purified by crystallization, flash chromatography or HPLC.

### General Procedure C

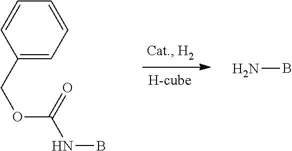

Cbz-protected amine is dissolved in a solvent (e.g. EtOH, ethyl acetate) or solvent mixtures at concentrations of e.g. 10 mg/ml depending on solubility. Hydrogenation by H-Cube® using a Pd/C or Raney-Ni catalyst cartridge at 50° C. Typically hydrogen pressure can be set from normal pressure to 50 bar and flow rates of 1 ml/min. If necessary hydrogenation is repeated until complete. Solvent is removed under reduced pressure to give a product usually pure enough to be used in the next reaction. If necessary the product can be purified by normal or revered phase flash chromatography.

### General Procedure D

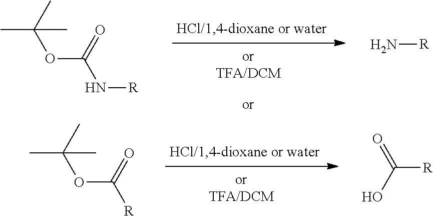

Boc-protected amine or tert-butyl carboxylate is dissolved in e.g. 4 N HCl in 1,4-dioxane, concentrated HCl in water or TFA in DCM (e.g. 20%) at room temperature or elevated temperature (e.g 60° C.) and stirred at room temperature. After reaction is found to be complete volatiles are removed under reduce pressure. The remaining crude is coevaporated with e.g. acetonitrile or THF to remove excess HCl to give the related ammonium slat or carboxylic acid pure enough to be used in the next reaction.

### General Procedure E

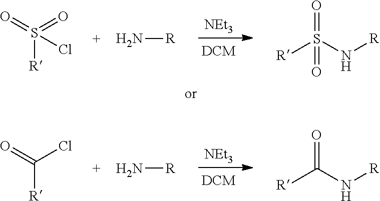

1 eq. amine and triethylamine (3 eq.) are dissolved in DCM (10-15 ml/mmol) and cooled to 0° C. Sulfonyl chloride or carboxylic acid chloride (1.3 eq.) is added and the mixture is stirred until the reaction was completed. For HPLC purification the reaction mixture was diluted with some methanol. Otherwise the reaction was diluted with ethyl acetate and washed with HCl aq. or Na₂CO₃ aq. and brine. After drying over Na₂SO₄ and removing the solvent under reduced pressure the residue was purified by normal or revered phase flash chromatography.

### General Procedure F

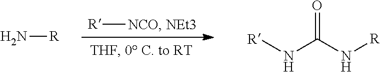

1 eq. amine and triethylamine (3 eq.) are dissolved in THF (10-15 ml/mmol) at RT. The related isocyanate (1.3 eq.) is added and the mixture is stirred until completion. The reaction mixture was diluted with some methanol and directly purified by HPLC.

### General Procedure G

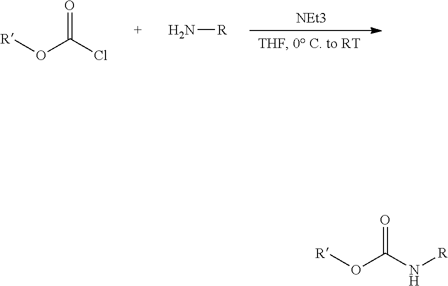

1 eq. amine and triethylamine (3 eq.) are dissolved in THF (10-15 ml/mmol) at 0° C. The related chloroformate (1.5 eq.) is added and the reaction was allowed to warm to room temperature. After complete reaction the reaction mixture was diluted with some methanol and directly purified by HPLC.

### General Procedure H

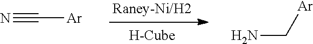

Aromatic nitrile in dissolved in ethanol, 2 M NH₃ in MeOH or other solvents or mixtures at concentrations of e.g. 10 mg/ml depending on solubility. Hydrogenation is achieved by H-Cube® using a Raney-Ni catalyst cartridge at elevated temperatures, e.g. 50-80° C. Typically hydrogen pressure can be set from 20-50 bar at flow rates of 1 ml/min. If necessary hydrogenation is repeated until complete. Solvent is removed under reduced pressure to give a product usually pure enough to be used in the next reaction. If necessary the product can be purified by normal or revered phase flash chromatography.

### Example A-1: Preparation of Compound 8

### (S)-tert-butyl 3-(((benzyloxy)carbonyl)amino)-4-(((S)-1-ethoxy-1-oxo-4-phenylbutan-2-yl)amino)-4-oxobutanoate (1)

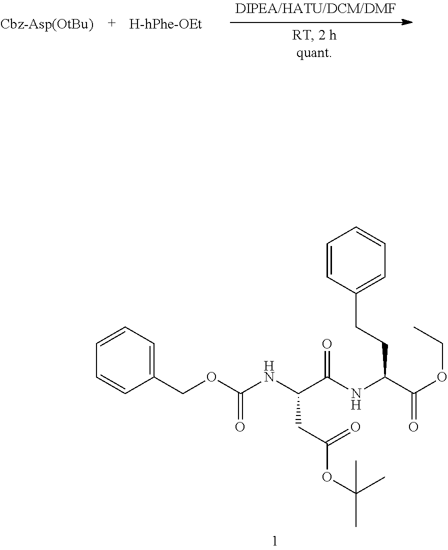 [SMILES: "Cbz-Asp(OtBu)" ; "+" ; "H-hPhe-OEt" ; CCOC(=O)[C@H](CCc1ccccc1)NC(=O)[C@H](CC(=O)OC(C)(C)C)NC(=O)OCc1ccccc1]

To a solution of Z-L-aspartic acid tert-butyl ester monohydrate (35 g, 102.5 mmol, 1.0 eq.) in DCM (1.2 L) and dry DMF (0.6 L) was added ethyl (S)-2-amino-4-phenylbutanoate hydrochloride (25 g, 102.5 mmol, 1.0 eq.) and HATU (57.3 g, 150.8 mmol, 1.5 eq.). Once purged under N₂, it was added dropwise and in 10 minutes DIPEA (44.4 g, 56.8 mL, 343.8 mmol, 3.4 equiv., previously filtered through a plug of Alox-basic). After two hours at room temperature, LC-MS monitoring showed none of the starting materials, so, after evaporation of the DCM under reduced pressure, the resulting residue was diluted with EtOAc/TBME 1:1 and then washed two times with saturated NaHCO₃ solution, five times with water and once with brine. The organic phase was dried over MgSO₄, filtered and the solvents evaporated under reduced pressure to obtain 60.7 g of crude 1. This crude was used in the next step without purification.

Formula: C₂₈H₃₆N₂O₇, exact mass: 512.3, found: 513.4 [M+H]⁺

### Preparation of (S)-2-((S)-2-(((benzyloxy)carbonyl)amino)-4-(tert-butoxy)-4-oxobutanamido)-4-phenylbutanoic acid (2)

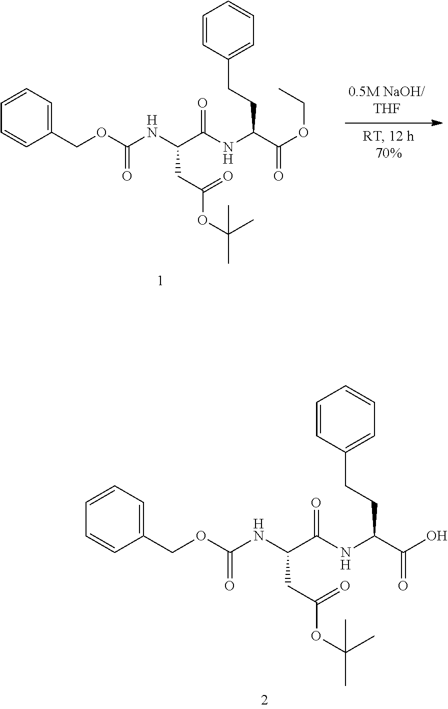 [SMILES: CCOC(=O)[C@H](CCc1ccccc1)NC(=O)[C@H](CC(=O)OC(C)(C)C)NC(=O)OCc1ccccc1 ; CC(C)(C)OC(=O)C[C@H](NC(=O)OCc1ccccc1)C(=O)N[C@@H](CCc1ccccc1)C(=O)O]

To a suspension of 1 (52 g, 101 mmol, 1.0 eq.) in THF (0.7 L) was added NaOH (0.5M, 202 mL, 101 mmol, 1.0 eq.) dropwise. The resulting mixture was stirred at room temperature for 12 hours before evaporation of the THF under reduced pressure. The resulting residue was diluted with water and TBME, and after decantation, the aqueous phase was further extracted two times with TBME. The resulting basic aqueous phase (pH ˜8) was acidified to pH 2 by addition of 10% HCl before extraction three times with DCM. The combined organic phase was dried over MgSO₄, filtered and evaporated to obtain 37 g of crude. This crude was purified in normal phase using Grace Reveleris and CHCl3/1% AcOH in MeOH as solvents to obtain 34.6 g of expected compound 2

Formula: C₂₆H₃₂N₂O₇, exact mass: 484.2, found: 485.3 [M+H]⁺

### tert-butyl 3-(2-(3-cyano-4-methylphenoxy)ethyl)piperidine-1-carboxylate (3)

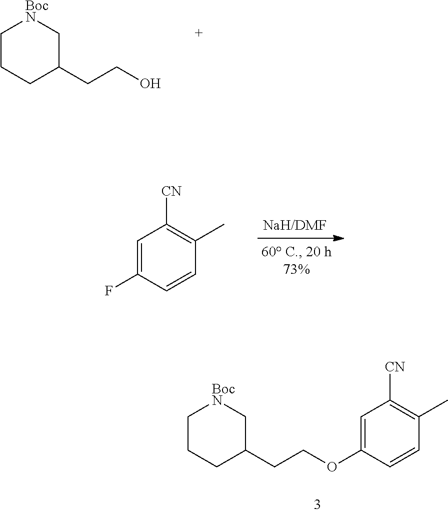

To a solution of N-Boc-3-(2-hydroxyethyl)piperidine (21 g, 91.7 mmol, 1.0 eq.) in dry DMF (180 mL) was added NaH (3.7 g, 60%, 91.7 mmol, 1.0 eq.). The resulting mixture was stirred for 30 minutes before the addition dropwise of a solution of 5-Fluoro-2-methylbenzonitrile (12.4 g, 91.7 mmol, 1.0 eq.) in dry DMF (40 mL). Once added, the mixture was heated at 60° C. After 20 hours of heating, LC-MS monitoring showed 78% conversion, so, after cooling to room temperature, additional NaH was added (3.7 g, 60%, 91.7 mmol, 1.0 eq.). The mixture was stirred again at room temperature for 30 minutes before heating at 60° C. for 7 hours. Once cooled, the reaction mixture was quenched carefully by addition of saturated NH₄Cl solution, and extracted three times with TBME. The combined organic phase was washed once with saturated NH₄Cl solution, once with saturated NaHCO₃ solution, three times with water and once with brine. Once dried over MgSO₄ and filtered, the solvent was evaporated under reduced pressure to obtain 33 g of crude. This crude was purified on normal phase using Grace Reveleris and DCM/CyH as solvents to obtain 26.9 g of expected compound 3.

Formula: C₂₀H₂₈N₂O₃, exact mass: 344.2, found: 345.1 [M+H]⁺

### tert-butyl 3-(2-(3-(aminomethyl)-4-methylphenoxy)ethyl)piperidine-1-carboxylate (4)

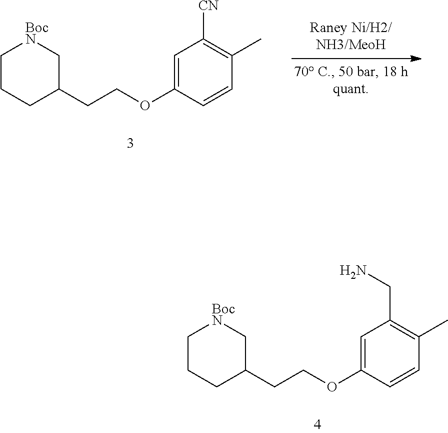 [SMILES: Cc1ccc(OCCC2CCCN(C(=O)OC(C)(C)C)C2)cc1C#N ; Cc1ccc(OCCC2CCCN(C(=O)OC(C)(C)C)C2)cc1CN]

To a solution of 3 (22 g, 63.9 mmol; 1.0 eq.) in 7N ammonia in MeOH (1.2 L) was added Raney-Nickel (22 g, which was washed previously two times with MeOH). The resulting mixture was stirred at 70° C. under 50 bar of H₂ for 18 hours. Once cooled, TLC monitoring showed no starting material, so, reaction mixture was filtered and the solid washed with MeOH. The combined filtrate was evaporated under reduced pressure to obtain 25 g of crude 4 as green oil that was used in the next step without purification.

### tert-butyl 3-(2-(3-((5S,8S)-5-(2-(tert-butoxy)-2-oxoethyl)-3,6,9-trioxo-8-phenethyl-1-phenyl-2-oxa-4,7,10-triazaundecan-11-yl)-4-methylphenoxy)ethyl)piperidine-1-carboxylate (5)

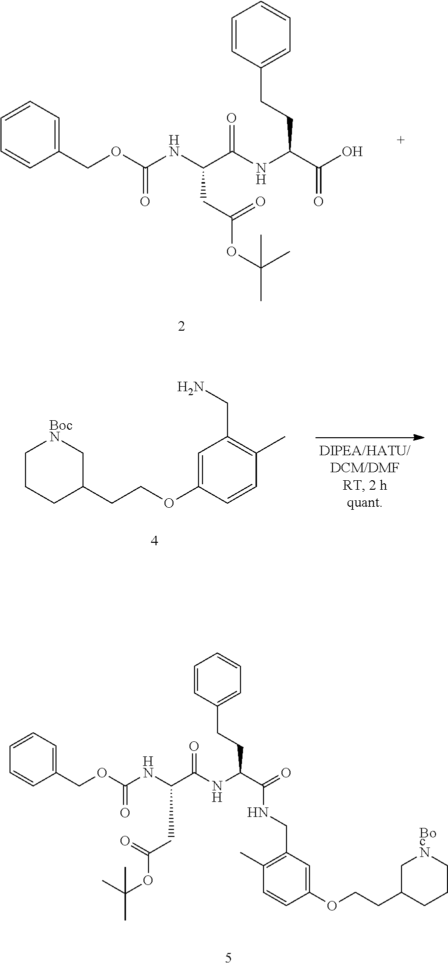 [SMILES: CC(C)(C)OC(=O)C[C@H](NC(=O)OCc1ccccc1)C(=O)N[C@@H](CCc1ccccc1)C(=O)O ; Cc1ccc(OCCC2CCCN(C(=O)OC(C)(C)C)C2)cc1CN ; Cc1ccc(OCCC2CCCN(C(=O)OC(C)(C)C)C2)cc1CNC(=O)[C@H](CCc1ccccc1)NC(=O)[C@H](CC(=O)OC(C)(C)C)NC(=O)OCc1ccccc1]

To a solution of 2 (34 g, 70.2 mmol, 1.0 eq.), 4 (24.5 g, 70.2 mmol, 1.0 eq.) and HATU (39.2 g, 103.2 mmol, 1.5 eq.) in DCM (0.86 L) and dry DMF (0.4 L) was added dropwise and in 20 minutes DIPEA (30.4 g, 38.9 mL, 242 mmol, 3.4 eq.). The reaction mixture was stirred at room temperature for 24 hours before evaporation of the DCM under reduced pressure. The resulting solution was added slowly over water (1.2 L) and then stirred at room temperature. The resulted upper layer was decanted and stirred again three times with more water. After final decantation, the slurry was dissolved with ACN and evaporated under reduced pressure. The resulted solid containing water was further co-evaporated three times once dissolved with ACN to obtain 62 g of crude. This crude was purified in normal phase using Grace Reveleris and DCM/CyH as solvents to obtain 43 g of expected compound 5.

Formula: C₄₆H₆₂N₄O₉, exact mass: 814.5, found: 815.6 [M+H]⁺

### (3S)-3-(((benzyloxy)carbonyl)amino)-4-(((2S)-1-((2-methyl-5-(2-(piperidin-3-yl)ethoxy)benzyl)amino)-1-oxo-4-phenylbutan-2-yl)amino)-4-oxobutanoic acid (6)

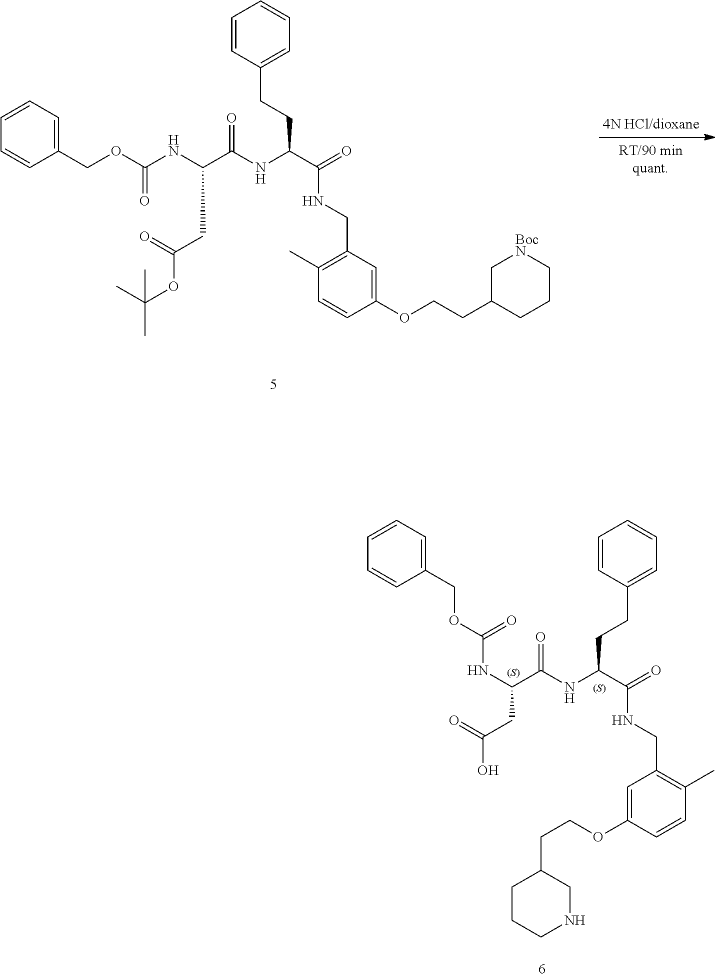

39.84 g (48.9 mmol) starting material 5 were dissolved in 40 ml 4 M HCl in dioxane and stirred at room temperature for 90 min. The solution was concentrated and stirred with additional 20 ml of 4 M HCl in dioxane at 60° C. for 1 h. Volatiles were removed under reduced pressure. Coevaporation with acetonitrile. Crude product 6 was used in the next step without further purification.

Formula: C₃₇H₄₆N₄O₇, exact mass: 658.3, found: 659.6 [M+H]⁺

### benzyl ((9S,12S)-5⁴-methyl-8,11,14-trioxo-9-phenethyl-4-oxa-7,10-diaza-1(3,1)-piperidina-5(1,3)-benzenacyclotetradecaphane-12-yl)carbamate (7)

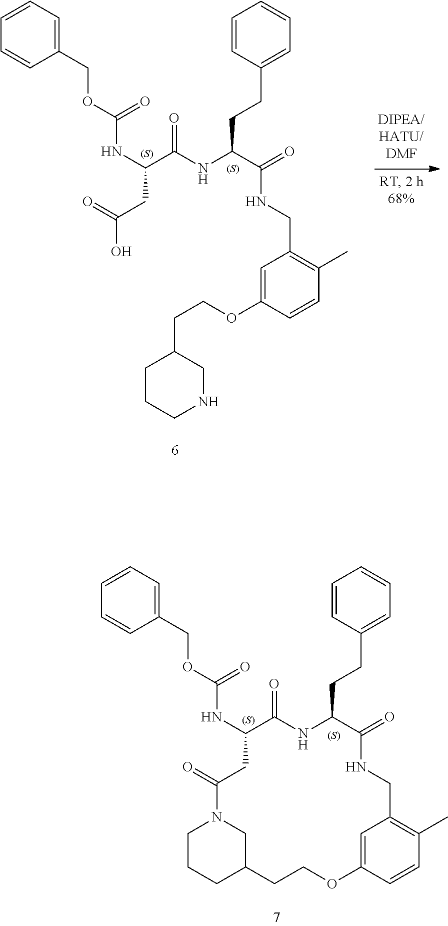

22.3 g (59 mmol) HATU were dissolved in 80 ml DMF. 48.9 mmol amino acid 6 in 114 ml DMF and 51.2 ml DIPEA were added dropwise at room temperature. The reaction mixture was distributed between ethyl acetate and 2 N NaOH solution. Some product precipitated and was collected and washed with MeOH. The organic phase was dried and evaporated. The remaining was triturated with methanol. Combined products summed up to 21.6 g of 7.

Formula: C₃₇H₄₄N₄O₆, exact mass: 640.3, found: 641.4 [M+H]⁺

In analogy to compound 7 the following diastereomers 289, 290, 291 and 292 were synthesized utilizing enantiomerically pure and commercially available building blocks as depicted in the related structures:

### benzyl ((13R,9R,12S)-5⁴-methyl-8,11,14-trioxo-9-phenethyl-4-oxa-7,10-diaza-1(3,1)-piperidina-5(1,3)-benzenacyclotetradecaphane-12-yl)carbamate (289)

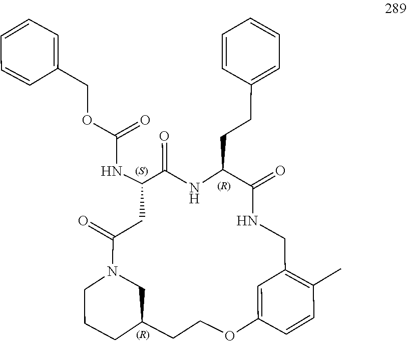

Formula: C₃₇H₄₄N₄O₆, exact mass: 640.3, found: 641.5 [M+H]⁺

### benzyl ((13R,9R,12R)-5⁴-methyl-8,11,14-trioxo-9-phenethyl-4-oxa-7,10-diaza-1(3,1)-piperidina-5(1,3)-benzenacyclotetradecaphane-12-yl)carbamate (290)

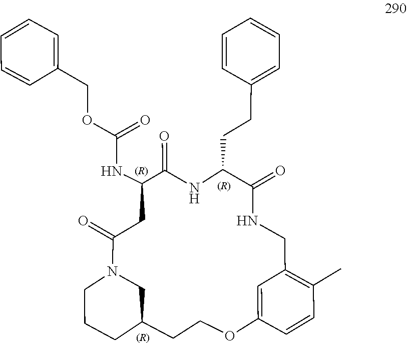

Formula: C₃₇H₄₄N₄O₆, exact mass: 640.3, found: 641.5 [M+H]⁺

### benzyl ((13R,9S,12R)-5⁴-methyl-8,11,14-trioxo-9-phenethyl-4-oxa-7,10-diaza-1(3,1)-piperidina-5(1,3)-benzenacyclotetradecaphane-12-yl)carbamate (291)

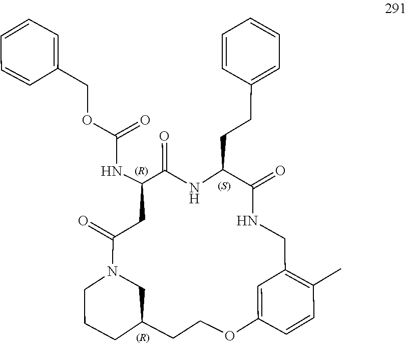

Formula: C₃₇H₄₄N₄O₆, exact mass: 640.3, found: 641.5 [M+H]⁺

### benzyl ((13R,9S,12S)-5⁴-methyl-8,11,14-trioxo-9-phenethyl-4-oxa-7,10-diaza-1(3,1)-piperidina-5(1,3)-benzenacyclotetradecaphane-12-yl)carbamate (292)

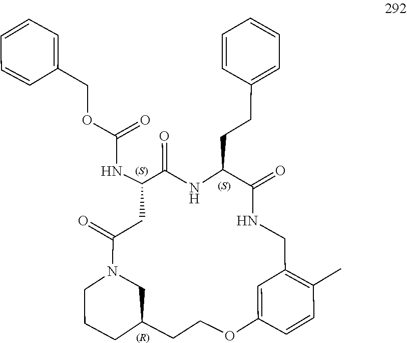

Formula: C₃₇H₄₄N₄O₆, exact mass: 640.3, found: 641.4 [M+H]⁺

### (9S,12S)-12-amino-5⁴-methyl-9-phenethyl-4-oxa-7,10-diaza-1(3,1)-piperidina-5(1,3)-benzenacyclotetradecaphane-8,11,14-trione (8)

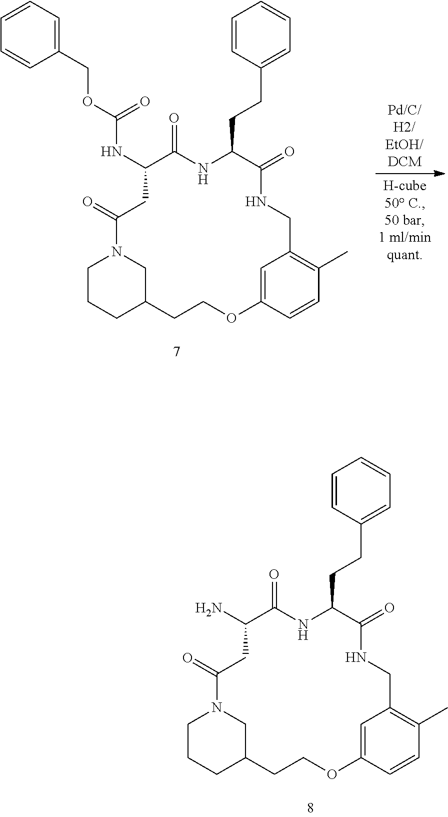 [SMILES: Cc1ccc2cc1CNC(=O)[C@H](CCc1ccccc1)NC(=O)[C@@H](NC(=O)OCc1ccccc1)CC(=O)N1CCCC(CCO2)C1 ; Cc1ccc2cc1CNC(=O)[C@H](CCc1ccccc1)NC(=O)[C@@H](N)CC(=O)N1CCCC(CCO2)C1]

6.8 g benzyl carbamate 7 were dissolved EtOH/DCM (5:1) and hydrogenated with an H-cube (ThalesNano), 10% Pd/C at 50° C. under 50 bar H₂ and a flow rate of 1 ml/min. 4 cycles were necessary to completely remove the protecting group. Quantitative yield after evaporation. Formula: C₂₉H₃₈N₄O₄, exact mass: 506.3, found: 507.3 [M+H]⁺

### Example A-2: Preparation of Macrocyclic Compounds 13, 16, 19 and 22

### tert-butyl 3-(2-(3-(((S)-2-(((benzyloxy)carbonyl)amino)-4-phenylbutanamido)methyl)-4-methylphenoxy)ethyl)piperidine-1-carboxylate (9)

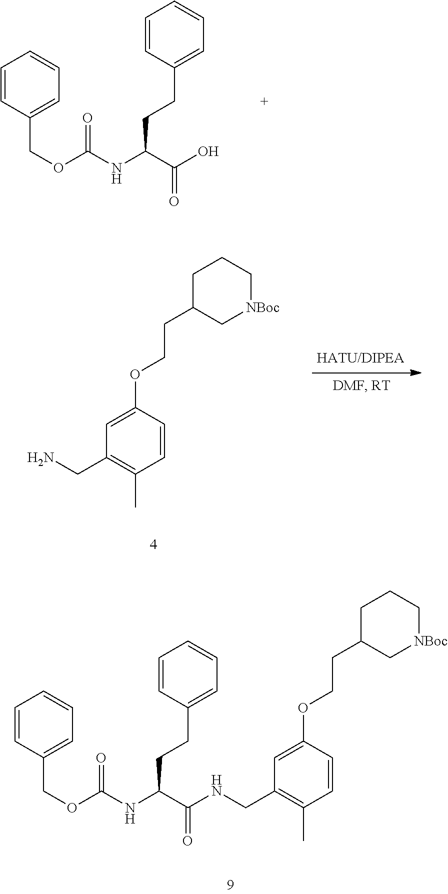 [SMILES: O=C(N[C@@H](CCc1ccccc1)C(=O)O)OCc1ccccc1 ; Cc1ccc(OCCC2CCCN(C(=O)OC(C)(C)C)C2)cc1CN ; Cc1ccc(OCCC2CCCN(C(=O)OC(C)(C)C)C2)cc1CNC(=O)[C@H](CCc1ccccc1)NC(=O)OCc1ccccc1]

Synthesis according to general procedure B with 473 mg amine 4 and 350 mg Cbz-hPhe. Purification was achieved by normal phase flash chromatography.

Formula: C₃₈H₄₉N₃O₆, exact mass: 643.4, found: 644.4 [M+H]⁺

### tert-butyl 3-(2-(3-(((S)-2-amino-4-phenylbutanamido)methyl)-4-methylphenoxy)ethyl) piperidine-1-carboxylate (10)

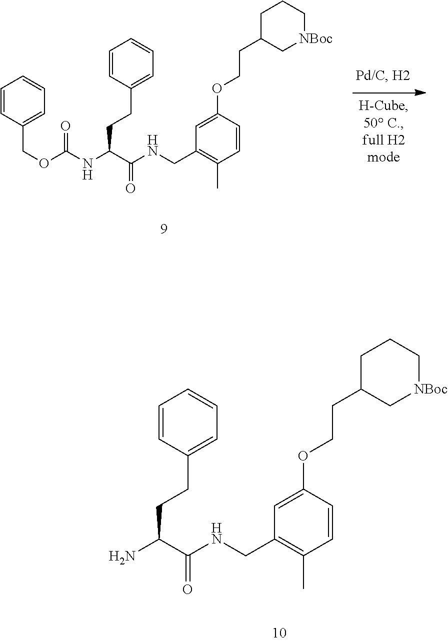 [SMILES: Cc1ccc(OCCC2CCCN(C(=O)OC(C)(C)C)C2)cc1CNC(=O)[C@H](CCc1ccccc1)NC(=O)OCc1ccccc1 ; Cc1ccc(OCCC2CCCN(C(=O)OC(C)(C)C)C2)cc1CNC(=O)[C@@H](N)CCc1ccccc1]

Following general procedure C using 260 mg Cbz-protected amine dissolved in 100 ml ethyl acetate/ethanol (1:1). H-Cube conditions: 1 ml/min, 50° C., full H2 mode. After removing of volatiles under reduced pressure the product was used without further purification.

### Amide Derivatives of Amine 10

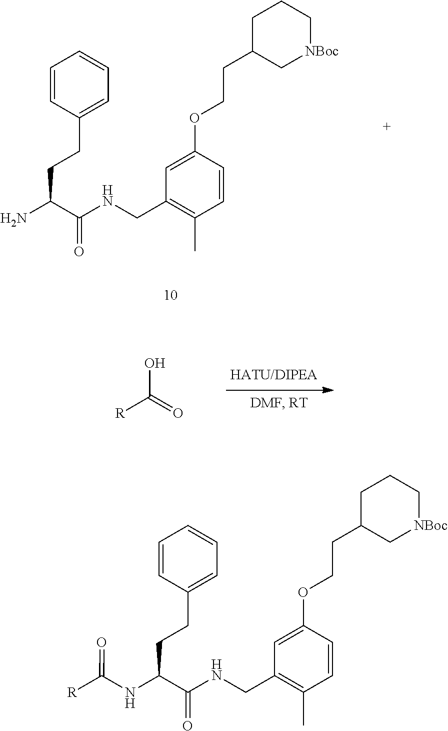

According to general procedure B with 75 mg amine 10. Purification was achieved by normal phase flash chromatography

TABLE A-1

| Cpd | Carboxylic | | | exact | [M + H]⁺ |
|---|---|---|---|---|---|
| No | acid | structure | name | mass | found |
| 11 | 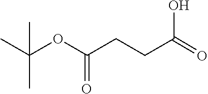 | 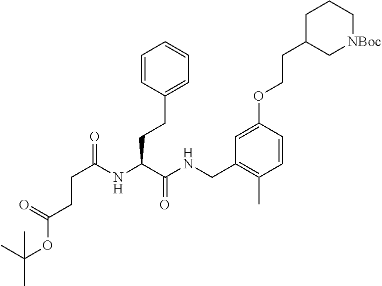 [SMILES: Cc1ccc(OCCC2CCCN(C(=O)OC(C)(C)C)C2)cc1CNC(=O)[C@H](CCc1ccccc1)NC(=O)CCC(=O)OC(C)(C)C] | tert-butyl 3-(2- (3-(((S)-2- (4- (tert- butoxy)-4- oxobutan- amido)- 4- phenyl- butanamido) methyl)-4- methyl- phenoxy) ethyl) piperidine- 1-carboxylate | 665.4 C₃₈H₅₅ N₃O₇ | 666.6 |
| 17 | 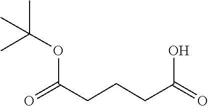 | 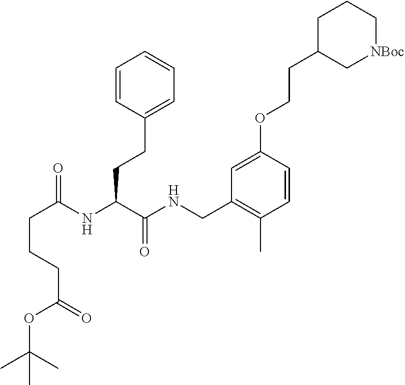 [SMILES: Cc1ccc(OCCC2CCCN(C(=O)OC(C)(C)C)C2)cc1CNC(=O)[C@H](CCc1ccccc1)NC(=O)CCCC(=O)OC(C)(C)C] | tert-butyl 3-(2- (3-(((S)-2- (5-(tert- butoxy)- 5-oxopentan- amido)- 4- phenyl- butanamido) methyl)-4- methyl- phenoxy) ethyl) piperidine- 1-carboxylate | 679.4 C₃₉H₅₇ N₃O₇ | 680.6 |
| 20 | 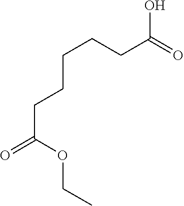 | 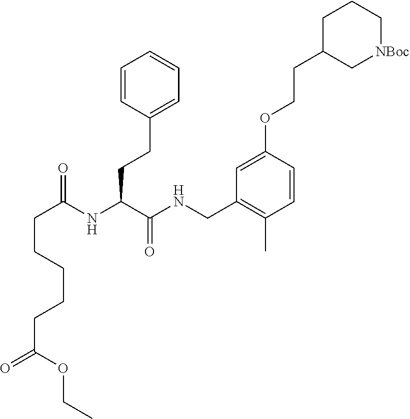 [SMILES: CCOC(=O)CCCCCC(=O)N[C@@H](CCc1ccccc1)C(=O)NCc1cc(OCCC2CCCN(C(=O)OC(C)(C)C)C2)ccc1C] | tert-butyl 3-(2- (3-(((S)-2- (7-ethoxy-7- oxoheptan- amido)- 4- phenylbutan- amido) methyl)-4- methyl- phenoxy) ethyl) piperidine- 1-carboxylate | 679.4 C₃₉H₅₇ N₃O₇ | 680.7 |
| 14 | 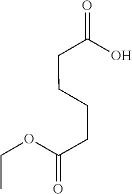 | 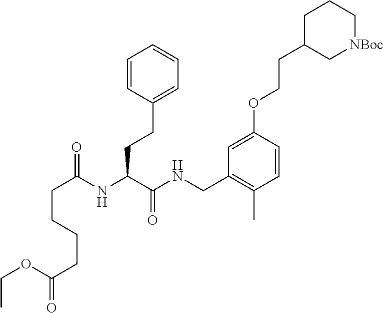 [SMILES: CCOC(=O)CCCCC(=O)N[C@@H](CCc1ccccc1)C(=O)NCc1cc(OCCC2CCCN(C(=O)OC(C)(C)C)C2)ccc1C] | tert-butyl 3-(2- (3-(((S)-2- (6-ethoxy-6- oxohexan- amido)-4- phenylbutan- amido) methyl)-4- methyl- phenoxy) ethyl) piperidine- 1-carboxylate | 665.4 C₃₈H₅₅ N₃O₇ | 666.8 |

Removal of Protecting Groups from Compounds 11, 14, 17 and 20

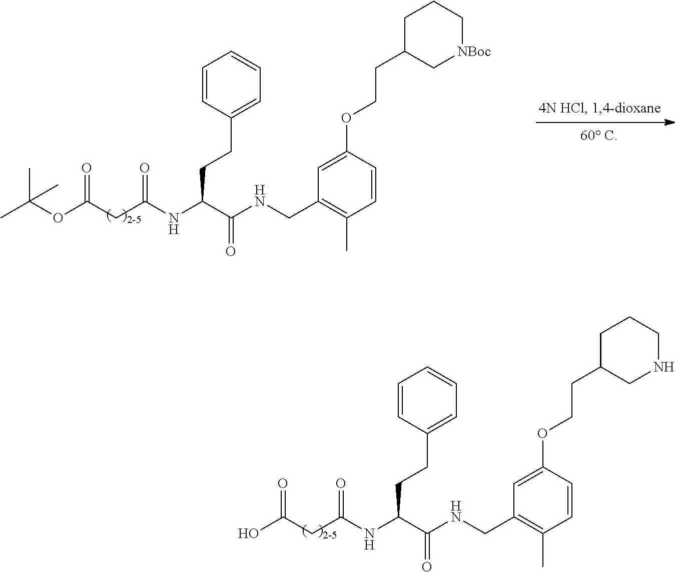

Protecting groups were cleaved according to general procedure D stirring compounds in 4 N HCl/1,4-dioxane at 60° C. for about 30 min

TABLE A-2

| Cpd | starting | | | exact | [M + H]⁺ |
|---|---|---|---|---|---|
| no | material | product structure | name | mass | found |
| 12 | 11 | 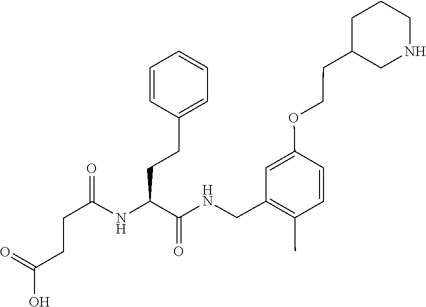 [SMILES: Cc1ccc(OCCC2CCCNC2)cc1CNC(=O)[C@H](CCc1ccccc1)NC(=O)CCC(=O)O] | 4-(((2S)-1-((2-methyl-5-(2- (piperidin-3- yl)ethoxy)benzyl)amino)-1- oxo-4-phenylbutan-2- yl)amino)-4-oxobutanoic acid | 509.3 C₂₉H₃₉ N₃O₅ | 510.2 |
| 18 | 17 | 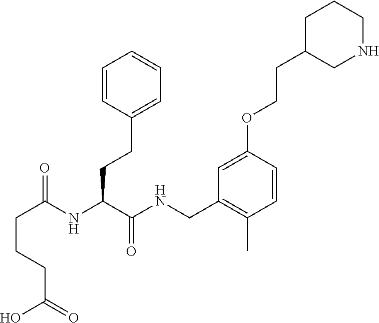 [SMILES: Cc1ccc(OCCC2CCCNC2)cc1CNC(=O)[C@H](CCc1ccccc1)NC(=O)CCCC(=O)O] | 5-(((2S)-1-((2-methyl-5-(2- (piperidin-3- yl)ethoxy)benzyl)amino)-1- oxo-4-phenylbutan-2- yl)amino)-5-oxopentanoic acid | 523.3 C₃₀H₄₁ N₃O₅ | 524.3 |
| 21 | 20 | 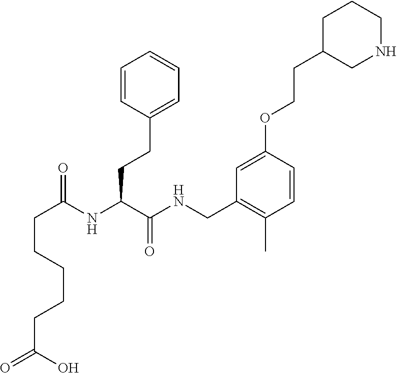 [SMILES: Cc1ccc(OCCC2CCCNC2)cc1CNC(=O)[C@H](CCc1ccccc1)NC(=O)CCCCCC(=O)O] | 7-(((2S)-1-((2-methyl-5-(2- (piperidin-3- yl)ethoxy)benzyl)amino)-1- oxo-4-phenylbutan-2- yl)amino)-7-oxoheptanoic acid | 551.3 C₃₂H₄₅ N₃O₅ | 552.3 |
| 15 | 14 | 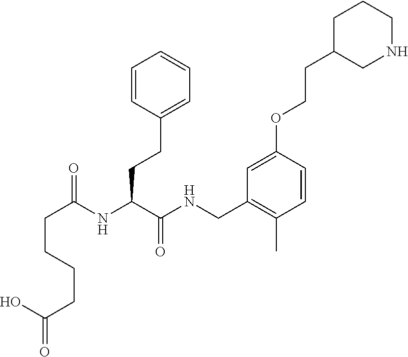 [SMILES: Cc1ccc(OCCC2CCCNC2)cc1CNC(=O)[C@H](CCc1ccccc1)NC(=O)CCCCC(=O)O] | 6-(((2S)-1-((2-methyl-5-(2- (piperidin-3- yl)ethoxy)benzyl)amino)-1- oxo-4-phenylbutan-2- yl)amino)-6-oxohexanoic acid | 537.3 C₃₁H₄₃ N₃O₅ | 538.7 |

### Macrocyclization of Amino Acids 12, 15, 18 and 21

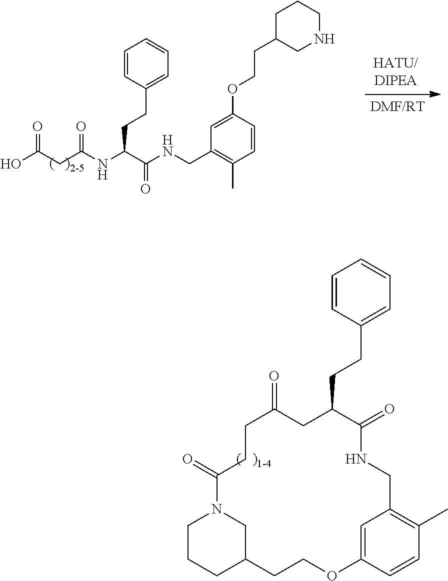

Macrocyclizations according to general procedure A. reaction mixtures were diluted with some methanol and purified via HPLC.

TABLE A-3

| Startingmaterial | | Product | | Cpd | exact | [M + H]⁺ |
|---|---|---|---|---|---|---|
| Cpd | Amount [mmol] | structure | name | no | mass | found |
| 12 | 0.09 | 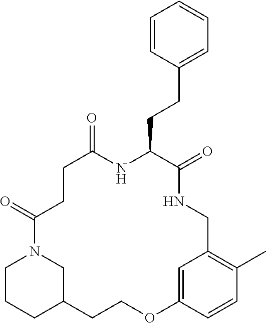 [SMILES: Cc1ccc2cc1CNC(=O)[C@H](CCc1ccccc1)NC(=O)CCC(=O)N1CCCC(CCO2)C1] | (9S)-5⁴-methyl-9- phenethyl-4-oxa-7,10- diaza-1(3,1)-piperidina- 5(1,3)- benzenacyclotetradecaphane- 8,11,14-trione | 13 | 491.3 C₂₉H₃₇ N₃O₄ | 492.5 |
| 18 | 0.171 | 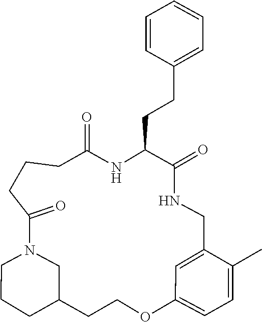 [SMILES: Cc1ccc2cc1CNC(=O)[C@H](CCc1ccccc1)NC(=O)CCCC(=O)N1CCCC(CCO2)C1] | (9S)-5⁴-methyl-9- phenethyl-4-oxa-7,10- diaza-1(3,1)-piperidina- 5(1,3)- benzenacyclopentadecaphane- 8,11,15-trione | 19 | 505.3 C₃₀H₃₉ N₃O₄ | 506.5 |
| 21 | 0.196 | 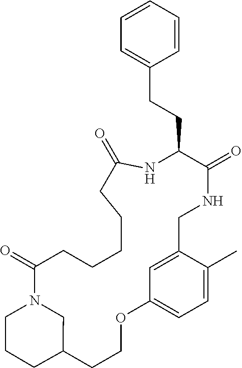 [SMILES: Cc1ccc2cc1CNC(=O)[C@H](CCc1ccccc1)NC(=O)CCCCCC(=O)N1CCCC(CCO2)C1] | (9S)-5⁴-methyl-9- phenethyl-4-oxa-7,10- diaza-1(3,1)-piperidina- 5(1,3)- benzenacycloheptadecaphane- 8,11,17-trione | 22 | 533.3 C₃₂H₄₃ N₃O₄ | 534.5 |
| 15 | 0.138 | 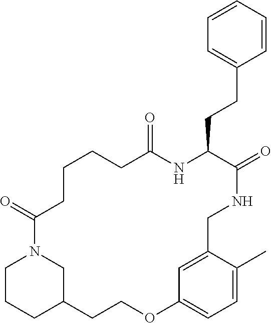 [SMILES: Cc1ccc2cc1CNC(=O)[C@H](CCc1ccccc1)NC(=O)CCCCC(=O)N1CCCC(CCO2)C1] | (9S)-5⁴-methyl-9- phenethyl-4-oxa-7,10- diaza-1(3,1)-piperidina- 5(1,3)- benzenacyclohexadecaphane- 8,11,16-trione | 16 | 519.3 C₃₁H₄₁ N₃O₄ | 520.5 |

### Example A-3: Preparation of Macrocyclic Compounds 25, 26, 27, 28, 29, 30, 31, 32, 33, 40, 41, 42, 60, 61, 62, 63, 64, 80, 81, 82, 83, 84, 85, 86, 87, 88, 89, 174, 175, 176, 177, 182, 219, 284, 285, 286, 287, 288, 323, 324, 327, 328, 329, 330, 331, 332, 333, 334, 335, 341, 369, 373

### tert-butyl 3-(2-(3-(((S)-2-((S)-4-ethoxy-2-(3-(3-fluorophenyl)propanamido)-4-oxobutanamido)-4-phenylbutanamido)methyl)-4-methylphenoxy)ethyl)piperidine-1-carboxylate (23)

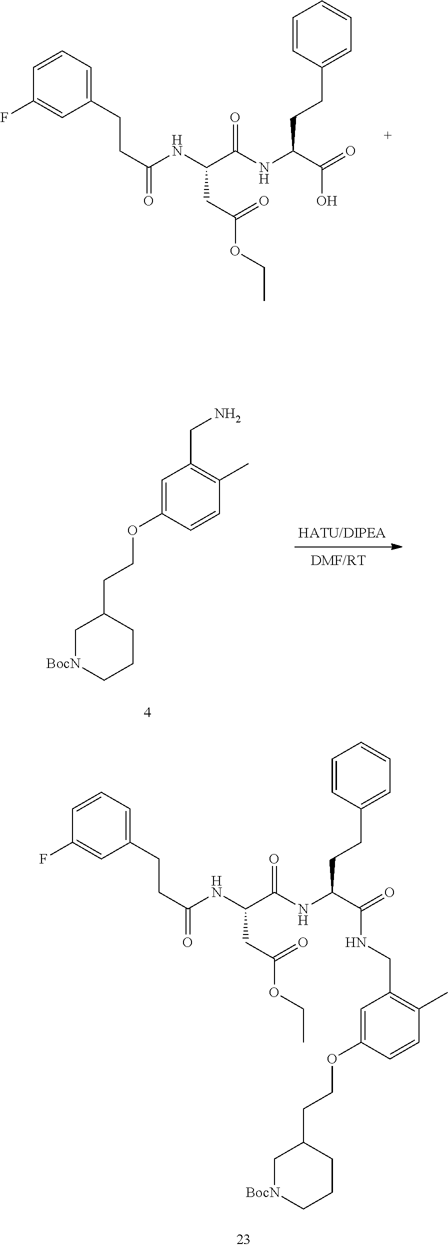 [SMILES: CCOC(=O)C[C@H](NC(=O)CCc1cccc(F)c1)C(=O)N[C@@H](CCc1ccccc1)C(=O)O ; Cc1ccc(OCCC2CCCN(C(=O)OC(C)(C)C)C2)cc1CN ; CCOC(=O)C[C@H](NC(=O)CCc1cccc(F)c1)C(=O)N[C@@H](CCc1ccccc1)C(=O)NCc1cc(OCCC2CCCN(C(=O)OC(C)(C)C)C2)ccc1C]

Amide coupling according to general procedure B with 113 mg carboxylic acid (prepared in analogy to compound 2) and 108 mg benzyl amine 4. Purification was achieved by normal phase flash chromatography applying a cyclohexane/ethyl acetate gradient.

Formula: C₄₅H₅₉FN₄O₈, exact mass: 802.4, found: 703.6 [M+H-Boc]+

### (3S)-3-(3-(3-fluorophenyl)propanamido)-4-(((2S)-1-((2-methyl-5-(2-(piperidin-3-yl)ethoxy)benzyl)amino)-1-oxo-4-phenylbutan-2-yl)amino)-4-oxobutanoic acid (24)

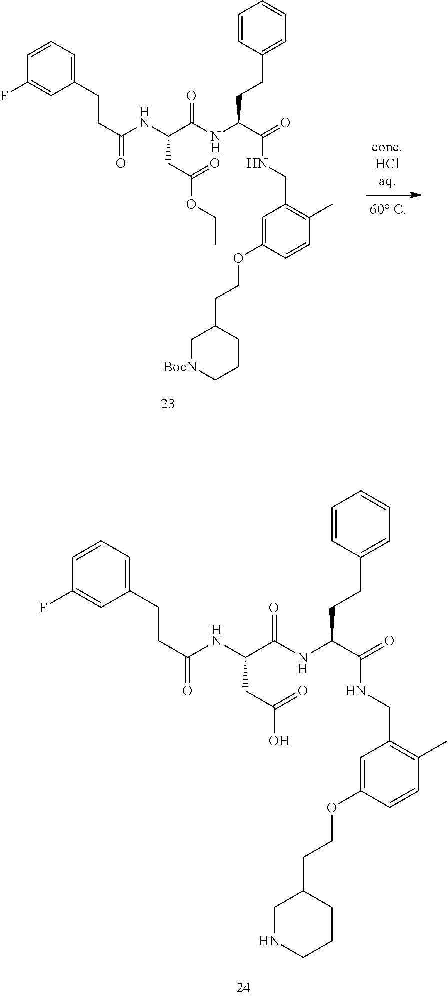 [SMILES: CCOC(=O)C[C@H](NC(=O)CCc1cccc(F)c1)C(=O)N[C@@H](CCc1ccccc1)C(=O)NCc1cc(OCCC2CCCN(C(=O)OC(C)(C)C)C2)ccc1C ; Cc1ccc(OCCC2CCCNC2)cc1CNC(=O)[C@H](CCc1ccccc1)NC(=O)[C@H](CC(=O)O)NC(=O)CCc1cccc(F)c1]

Both protecting groups were cleaved according to general procedure D in concentrated HCl in water at 60° C. for 90 min.

Formula: C₃₈H₄₇FN₄O₆, exact mass: 674.3, found: 675.2 [M+H]+

### 3-(3-fluorophenyl)-N-((9S,12S)-5⁴-methyl-8,11,14-trioxo-9-phenethyl-4-oxa-7,10-diaza-1(3,1)-piperidina-5(1,3)-benzenacyclotetradecaphane-12-yl)propanamide (25)

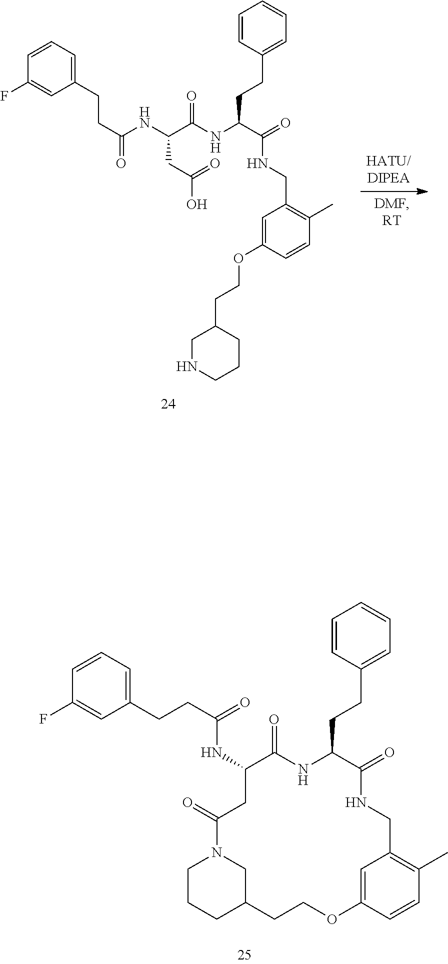 [SMILES: Cc1ccc(OCCC2CCCNC2)cc1CNC(=O)[C@H](CCc1ccccc1)NC(=O)[C@H](CC(=O)O)NC(=O)CCc1cccc(F)c1 ; Cc1ccc2cc1CNC(=O)[C@H](CCc1ccccc1)NC(=O)[C@@H](NC(=O)CCc1cccc(F)c1)CC(=O)N1CCCC(CCO2)C1]

0.146 mmol amino acid 24 were cyclized according to general procedure A. Purification via RP18 flash chromatography.

Formula: C₃₈H₄₅FN₄O₅, exact mass: 656.3, found: 657.3 [M+H]+

### Amide Derivatizations of Compound 8 According to General Procedure B

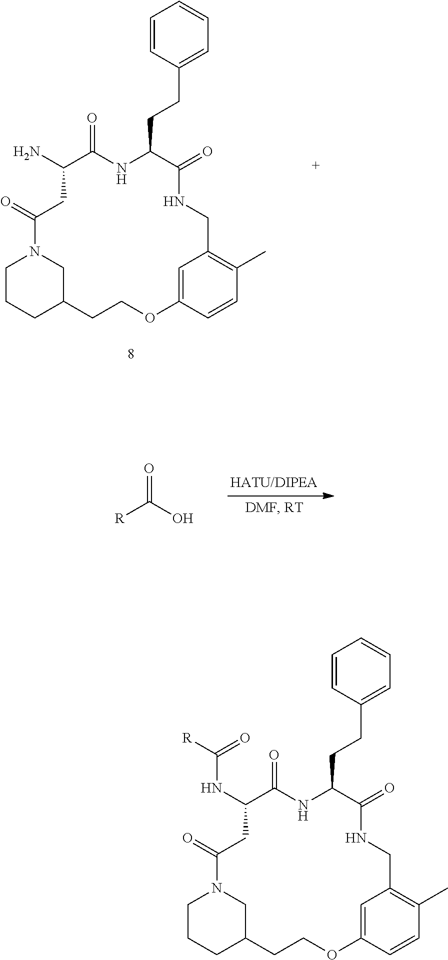

Amine 8 (e.g. 20 mg) was coupled with carboxylic acids according to general procedure B to give amides disclosed in table below. Purifications were achieved by HPLC or reversed phase flash chromatography.

TABLE A-4

| Cpd | | | Exact | [M + H]⁺ |
|---|---|---|---|---|
| No | structure | name | mass | found |
| 26 | 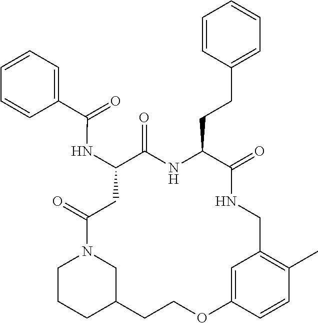 [SMILES: Cc1ccc2cc1CNC(=O)[C@H](CCc1ccccc1)NC(=O)[C@@H](NC(=O)c1ccccc1)CC(=O)N1CCCC(CCO2)C1] | N-((9S,12S)-5⁴-methyl- 8,11,14-trioxo-9-phenethyl- 4-oxa-7,10-diaza-1(3,1)- piperidina-5(1,3)- benzenacyclotetradecaphane- 12-yl)benzamide | 610.3 C₃₆H₄₂N₄O₅ | 611.4 |
| 27 | 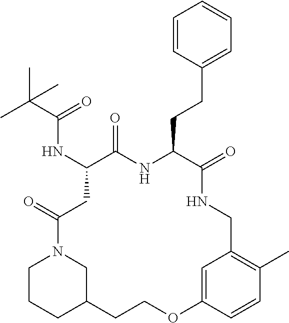 [SMILES: Cc1ccc2cc1CNC(=O)[C@H](CCc1ccccc1)NC(=O)[C@@H](NC(=O)C(C)(C)C)CC(=O)N1CCCC(CCO2)C1] | N-((9S,12S)-5⁴-methyl- 8,11,14-trioxo-9-phenethyl- 4-oxa-7,10-diaza-1(3,1)- piperidina-5(1,3)- benzenacyclotetradecaphane- 12-yl)pivalamide | 590.3 C₃₄H₄₆N₄O₅ | 591.4 |
| 28 | 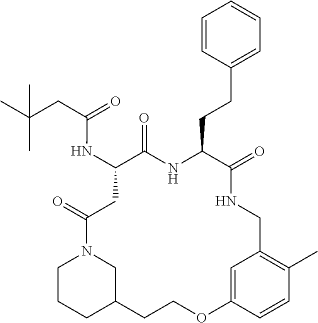 [SMILES: Cc1ccc2cc1CNC(=O)[C@H](CCc1ccccc1)NC(=O)[C@@H](NC(=O)CC(C)(C)C)CC(=O)N1CCCC(CCO2)C1] | 3,3-dimethyl-N-((9S,12S)- 5⁴-methyl-8,11,14-trioxo- 9-phenethyl-4-oxa-7,10- diaza-1(3,1)-piperidina-5(1,3)- benzenacyclotetradecaphane- 12-yl)butanamide | 604.4 C₃₅H₄₈N₄O₅ | 605.4 |
| 29 | 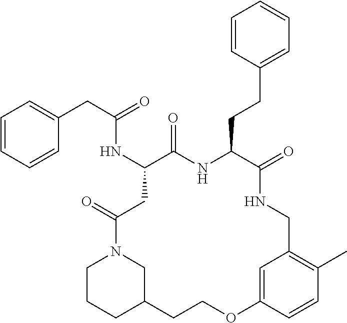 [SMILES: Cc1ccc2cc1CNC(=O)[C@H](CCc1ccccc1)NC(=O)[C@@H](NC(=O)Cc1ccccc1)CC(=O)N1CCCC(CCO2)C1] | N-((9S,12S)-5⁴-methyl- 8,11,14-trioxo-9-phenethyl- 4-oxa-7,10-diaza-1(3,1)- piperidina-5(1,3)- benzenacyclotetradecaphane- 12-yl)-2-phenylacetamide | 624.3 C₃₇H₄₄N₄O₅ | 625.5 |
| 30 | 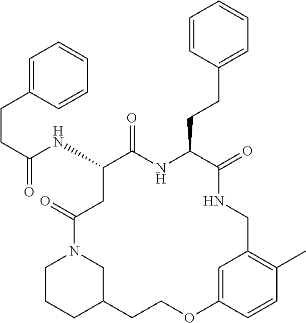 [SMILES: Cc1ccc2cc1CNC(=O)[C@H](CCc1ccccc1)NC(=O)[C@@H](NC(=O)CCc1ccccc1)CC(=O)N1CCCC(CCO2)C1] | N-((9S,12S)-5⁴-methyl- 8,11,14-trioxo-9-phenethyl- 4-oxa-7,10-diaza-1(3,1)- piperidina-5(1,3)- benzenacyclotetradecaphane- 12-yl)-3-phenylpropanamide | 638.3 C₃₈H₄₆N₄O₅ | 639.5 |
| 31 | 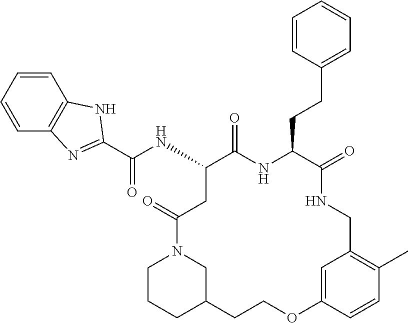 [SMILES: Cc1ccc2cc1CNC(=O)[C@H](CCc1ccccc1)NC(=O)[C@@H](NC(=O)c1nc3ccccc3[nH]1)CC(=O)N1CCCC(CCO2)C1] | N-((9S,12S)-5⁴-methyl- 8,11,14-trioxo-9-phenethyl- 4-oxa-7,10-diaza-1(3,1)- piperidina-5(1,3)- benzenacyclotetradecaphane- 12-yl)-1H-benzo[d] imidazole-2-carboxamide | 650.3 C₃₇H₄₂N₆O₅ | 651.1 |
| 32 | 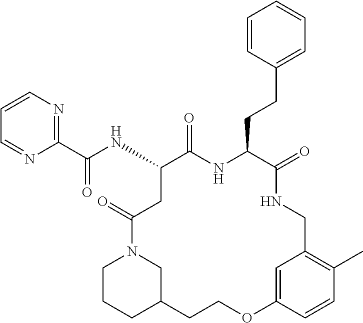 [SMILES: Cc1ccc2cc1CNC(=O)[C@H](CCc1ccccc1)NC(=O)[C@@H](NC(=O)c1ncccn1)CC(=O)N1CCCC(CCO2)C1] | N-((9S,12S)-5⁴-methyl- 8,11,14-trioxo-9-phenethyl- 4-oxa-7,10-diaza-1(3,1)- piperidina-5(1,3)- benzenacyclotetradecaphane- 12-yl)pyrimidine-2- carboxamide | 612.3 C₃₄H₄₀N₆O₅ | 613.4 |
| 33 | 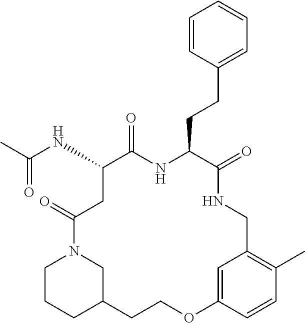 [SMILES: CC(=O)N[C@H]1CC(=O)N2CCCC(CCOc3ccc(C)c(c3)CNC(=O)[C@H](CCc3ccccc3)NC1=O)C2] | N-((9S,12S)-5⁴-methyl- 8,11,14-trioxo-9-phenethyl- 4-oxa-7,10-diaza-1(3,1)- piperidina-5(1,3)- benzenacyclotetradecaphane- 12-yl)acetamide | 548.3 C₃₁H₄₀N₄O₅ | 549.3 |
| 40 | 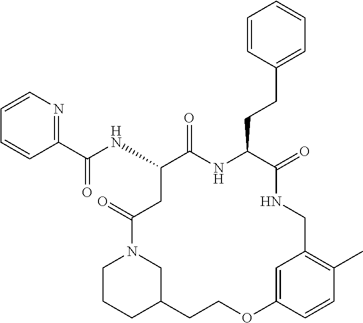 [SMILES: Cc1ccc2cc1CNC(=O)[C@H](CCc1ccccc1)NC(=O)[C@@H](NC(=O)c1ccccn1)CC(=O)N1CCCC(CCO2)C1] | N-((9S,12S)-5⁴-methyl- 8,11,14-trioxo-9-phenethyl- 4-oxa-7,10-diaza-1(3,1)- piperidina-5(1,3)- benzenacyclotetradecaphane- 12-yl)picolinamide | 611.3 C₃₅H₄₁N₅O₅ | 612.4 |
| 41 | 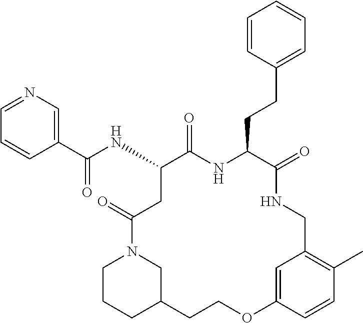 [SMILES: Cc1ccc2cc1CNC(=O)[C@H](CCc1ccccc1)NC(=O)[C@@H](NC(=O)c1cccnc1)CC(=O)N1CCCC(CCO2)C1] | N-((9S,12S)-5⁴-methyl- 8,11,14-trioxo-9-phenethyl- 4-oxa-7,10-diaza-1(3,1)- piperidina-5(1,3)- benzenacyclotetradecaphane- 12-yl)nicotinamide | 611.3 C₃₅H₄₁N₅O₅ | 612.4 |
| 42 | 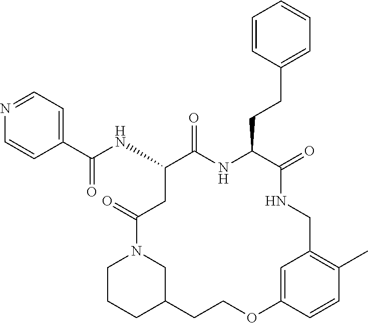 [SMILES: Cc1ccc2cc1CNC(=O)[C@H](CCc1ccccc1)NC(=O)[C@@H](NC(=O)c1ccncc1)CC(=O)N1CCCC(CCO2)C1] | N-((9S,12S)-5⁴-methyl- 8,11,14-trioxo-9-phenethyl- 4-oxa-7,10-diaza-1(3,1)- piperidina-5(1,3)- benzenacyclotetradecaphane- 12-yl)isonicotinamide | 611.3 C₃₅H₄₁N₅O₅ | 612.3 |
| 60 | 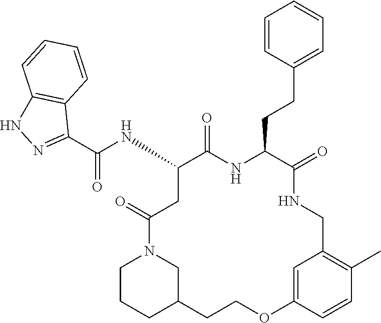 [SMILES: Cc1ccc2cc1CNC(=O)[C@H](CCc1ccccc1)NC(=O)[C@@H](NC(=O)c1n[nH]c3ccccc13)CC(=O)N1CCCC(CCO2)C1] | N-((9S,12S)-5⁴-methyl- 8,11,14-trioxo-9-phenethyl- 4-oxa-7,10-diaza-1(3,1)- piperidina-5(1,3)- benzenacyclotetradecaphane- 12-yl)-1H-indazole- 3-carboxamide | 650.3 C₃₇H₄₂N₆O₅ | 651.5 |
| 61 | 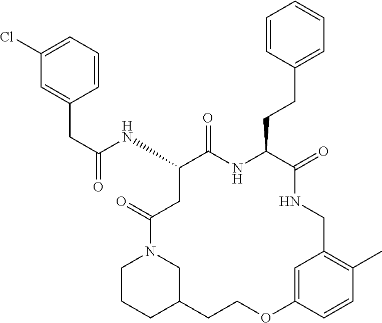 [SMILES: Cc1ccc2cc1CNC(=O)[C@H](CCc1ccccc1)NC(=O)[C@@H](NC(=O)Cc1cccc(Cl)c1)CC(=O)N1CCCC(CCO2)C1] | 2-(3-chlorophenyl)-N- ((9S,12S)-54- methyl-8,11,14-trioxo- 9-phenethyl-4-oxa-7,10- diaza-1(3,1)-piperidina-5(1,3)- benzenacyclotetradecaphane- 12-yl)acetamide | 658.3 C₃₇H₄₃ClN₄O₅ | 659.3 |
| 62 | 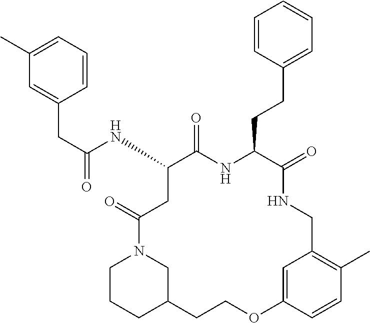 | N-((9S,12S)-5⁴-methyl- 8,11,14-trioxo-9-phenethyl- 4-oxa-7,10-diaza-1(3,1)- piperidina-5(1,3)- benzenacyclotetradecaphane- 12-yl)-2-(m-tolyl)acetamide | 638.3 C₃₈H₄₆N₄O₅ | 639.4 |
| 63 | 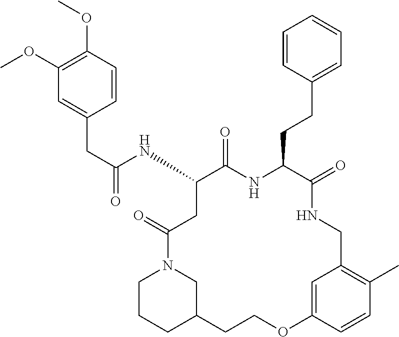 | 2-(3,4-dimethoxyphenyl)- N-((9S,12S)-5⁴- methyl-8,11,14-trioxo- 9-phenethyl-4-oxa-7,10- diaza-1(3,1)-piperidina-5(1,3)- benzenacyclotetradecaphane- 12-yl)acetamide | 684.4 C₃₉H₄₈N₄O₇ | 685.4 |
| 64 | 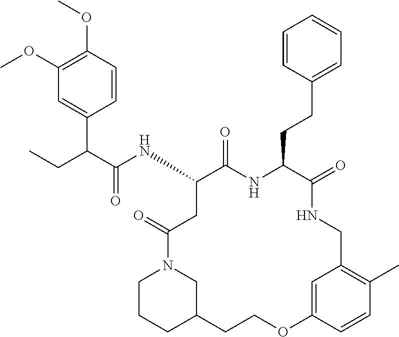 | 2-(3,4-dimethoxyphenyl)- N-((9S,12S)-5⁴- methyl-8,11,14-trioxo- 9-phenethyl-4-oxa-7,10- diaza-1(3,1)-piperidina-5(1,3)- benzenacyclotetradecaphane- 12-yl)butanamide | 712.4 C₄₁H₅₂N₄O₇ | 713.4 |
| 80 | 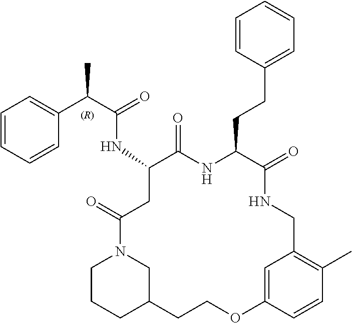 | (2R)-N-((9S,12S)-5⁴- methyl-8,11,14- trioxo-9-phenethyl-4- oxa-7,10-diaza- 1(3,1)-piperidina-5(1,3)- benzenacyclotetradecaphane- 12-yl)-2-phenylpropanamide | 638.3 C₃₈H₄₆N₄O₅ | 639.4 |
| 81 | 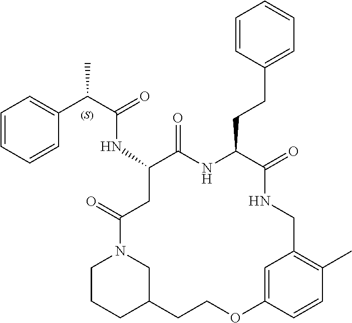 | (2S)-N-((9S,12S)-5⁴- methyl-8,11,14- trioxo-9-phenethyl-4- oxa-7,10-diaza- 1(3,1)-piperidina-5(1,3)- benzenacyclotetradecaphane- 12-yl)-2-phenylpropanamide | 638.3 C₃₈H₄₆N₄O₅ | 639.4 |
| 82 | 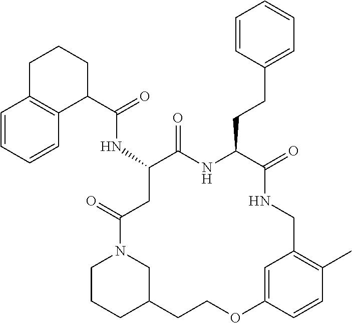 [SMILES: Cc1ccc2cc1CNC(=O)[C@H](CCc1ccccc1)NC(=O)[C@@H](NC(=O)C1CCCc3ccccc31)CC(=O)N1CCCC(CCO2)C1] | N-((9S,12S)-5⁴-methyl- 8,11,14-trioxo-9-phenethyl- 4-oxa-7,10-diaza-1(3,1)- piperidina-5(1,3)- benzenacyclotetradecaphane- 12-yl)-1,2,3,4- tetrahydronaphthalene-1- carboxamide | 664.4 C₄₀H₄₈N₄O₅ | 665.4 |
| 83 | 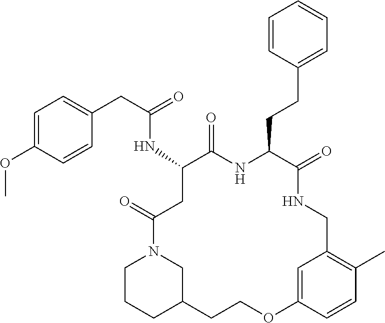 [SMILES: COc1ccc(CC(=O)N[C@H]2CC(=O)N3CCCC(CCOc4ccc(C)c(c4)CNC(=O)[C@H](CCc4ccccc4)NC2=O)C3)cc1] | 2-(4-methoxyphenyl)-N- ((9S,12S)-5⁴- methyl-8,11,14-trioxo- 9-phenethyl-4-oxa-7,10-diaza- 1(3,1)-piperidina-5(1,3)- benzenacyclotetradecaphane- 12-yl)acetamide | 654.3 C₃₈H₄₆N₄O₆ | 655.4 |
| 84 | 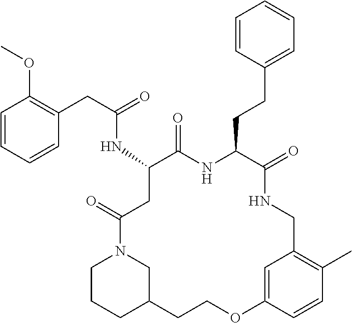 [SMILES: COc1ccccc1CC(=O)N[C@H]1CC(=O)N2CCCC(CCOc3ccc(C)c(c3)CNC(=O)[C@H](CCc3ccccc3)NC1=O)C2] | 2-(2-methoxyphenyl)-N- ((9S,12S)-5⁴- methyl-8,11,14-trioxo- 9-phenethyl-4-oxa-7,10-diaza- 1(3,1)-piperidina-5(1,3)- benzenacyclotetradecaphane- 12-yl)acetamide | 654.3 C₃₈H₄₆N₄O₆ | 655.3 |
| 85 | 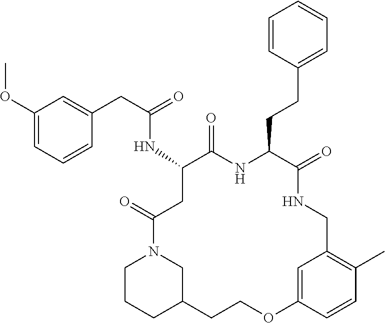 [SMILES: COc1cccc(CC(=O)N[C@H]2CC(=O)N3CCCC(CCOc4ccc(C)c(c4)CNC(=O)[C@H](CCc4ccccc4)NC2=O)C3)c1] | 2-(3-methoxyphenyl)-N- ((9S,12S)-5⁴- methyl-8,11,14-trioxo- 9-phenethyl-4-oxa-7,10-diaza- 1(3,1)-piperidina-5(1,3)- benzenacyclotetradecaphane- 12-yl)acetamide | 654.3 C₃₈H₄₆N₄O₆ | 655.4 |
| 86 | 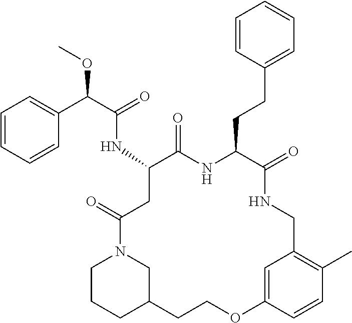 [SMILES: CO[C@@H](C(=O)N[C@H]1CC(=O)N2CCCC(CCOc3ccc(C)c(c3)CNC(=O)[C@H](CCc3ccccc3)NC1=O)C2)c1ccccc1] | (2R)-2-methoxy-N- ((9S,12S)-5⁴-methyl-8,11,14- trioxo-9-phenethyl-4-oxa-7,10- diaza-1(3,1)-piperidina-5(1,3)- benzenacyclotetradecaphane- 12-yl)-2-phenylacetamide | 654.3 C₃₈H₄₆N₄O₆ | 655.4 |
| 87 | 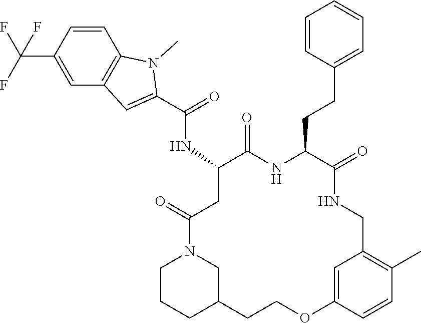 [SMILES: Cc1ccc2cc1CNC(=O)[C@H](CCc1ccccc1)NC(=O)[C@@H](NC(=O)c1cc3cc(C(F)(F)F)ccc3n1C)CC(=O)N1CCCC(CCO2)C1] | 1-methyl-N-((9S,12S)- 5⁴-methyl-8,11,14-trioxo- 9-phenethyl-4-oxa-7,10-diaza- 1(3,1)-piperidina-5(1,3)- benzenacyclotetradecaphane- 12-yl)-5-(trifluoromethyl)-1H- indole-2-carboxamide | 731.3 C₄₀H₄₄F₃N₅O₅ | 732.4 |
| 88 | 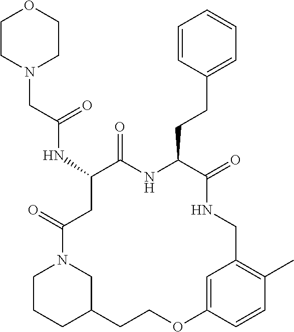 [SMILES: Cc1ccc2cc1CNC(=O)[C@H](CCc1ccccc1)NC(=O)[C@@H](NC(=O)CN1CCOCC1)CC(=O)N1CCCC(CCO2)C1] | N-((9S,12S)-5⁴-methyl- 8,11,14-trioxo-9-phenethyl- 4-oxa-7,10-diaza-1(3,1)- piperidina-5(1,3)- benzenacyclotetradecaphane- 12-yl)-2-morpholinoacetamide | 633.4 C₃₅H₄₇N₅O₆ | 634.4 |
| 89 | 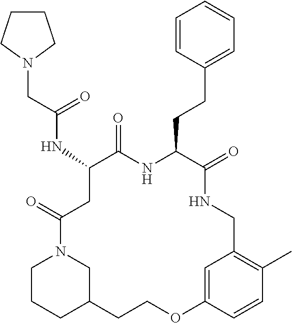 [SMILES: Cc1ccc2cc1CNC(=O)[C@H](CCc1ccccc1)NC(=O)[C@@H](NC(=O)CN1CCCC1)CC(=O)N1CCCC(CCO2)C1] | N-((9S,12S)-5⁴-methyl- 8,11,14-trioxo-9-phenethyl- 4-oxa-7,10-diaza-1(3,1)- piperidina-5(1,3)- benzenacyclotetradecaphane- 12-yl)-2-(pyrrolidin-1- yl)acetamide | 617.4 C₃₅H₄₇N₅O₅ | 618.4 |
| 174 | 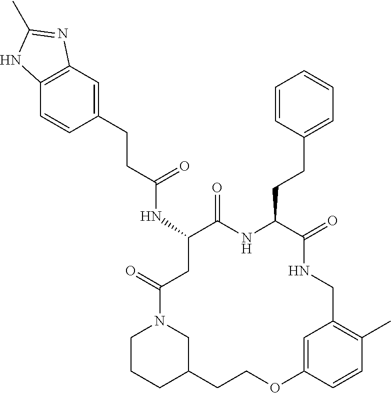 | 3-(2-methyl-1H-benzo[d] imidazol-6-yl)-N-((9S,12S)- 5⁴-methyl-8,11,14-trioxo-9- phenethyl-4-oxa-7,10-diaza- 1(3,1)-piperidina-5(1,3)- benzenacyclotetradecaphane- 12-yl)propanamide | 692.4 C₄₀H₄₈N₆O₅ | 693.4 |
| 175 | 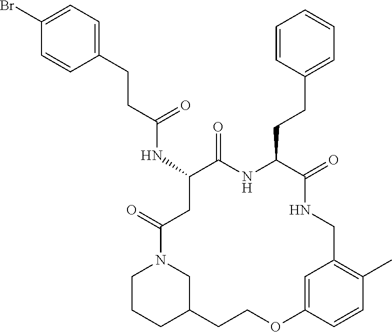 [SMILES: Cc1ccc2cc1CNC(=O)[C@H](CCc1ccccc1)NC(=O)[C@@H](NC(=O)CCc1ccc(Br)cc1)CC(=O)N1CCCC(CCO2)C1] | 3-(4-bromophenyl)-N- ((9S,12S)-5⁴- methyl-8,11,14-trioxo- 9-phenethyl-4-oxa-7,10- diaza-1(3,1)-piperidina-5(1,3)- benzenacyclotetradecaphane- 12-yl)propanamide | 716.3 C₃₈H₄₅BrN₄O₅ | 717.3 |
| 176 | 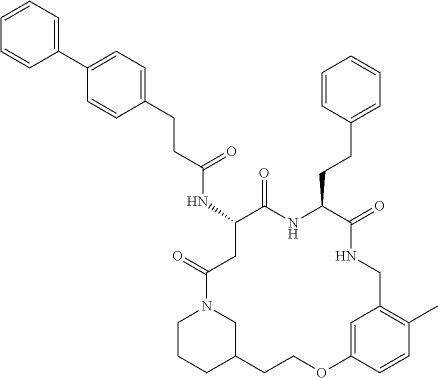 [SMILES: Cc1ccc2cc1CNC(=O)[C@H](CCc1ccccc1)NC(=O)[C@@H](NC(=O)CCc1ccc(-c3ccccc3)cc1)CC(=O)N1CCCC(CCO2)C1] | 3-([1,1′-biphenyl]-4- yl)-N-((9S,12S)-5⁴- methyl-8,11,14-trioxo- 9-phenethyl-4-oxa-7,10- diaza-1(3,1)-piperidina-5(1,3)- benzenacyclotetradecaphane- 12-yl)propanamide | 714.4 C₄₄H₅₀N₄O₅ | 715.4 |
| 177 | 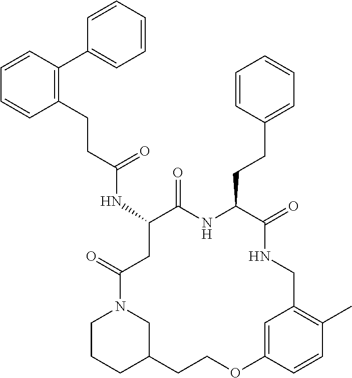 [SMILES: Cc1ccc2cc1CNC(=O)[C@H](CCc1ccccc1)NC(=O)[C@@H](NC(=O)CCc1ccccc1-c1ccccc1)CC(=O)N1CCCC(CCO2)C1] | N-((9S,12S)-5⁴-methyl- 8,11,14-trioxo-9-phenethyl- 4-oxa-7,10-diaza-1(3,1)- piperidina-5(1,3)- benzenacyclotetradecaphane- 12-yl)-3-(2-phenoxyphenyl) propanamide | 714.4 C₄₄H₅₀N₄O₅ | 715.4 |
| 182 | 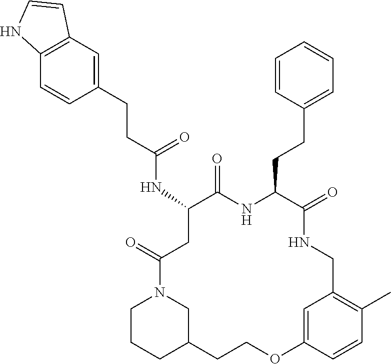 [SMILES: Cc1ccc2cc1CNC(=O)[C@H](CCc1ccccc1)NC(=O)[C@@H](NC(=O)CCc1ccc3[nH]ccc3c1)CC(=O)N1CCCC(CCO2)C1] | 3-(1H-indol-5-yl)-N- ((9S,12S)-5⁴-methyl-8,11,14- trioxo-9-phenethyl-4-oxa-7,10- diaza-1(3,1)-piperidina-5(1,3)- benzenacyclotetradecaphane- 12-yl)propanamide | 677.4 C₄₀H₄₇N₅O₅ | 678.5 |
| 219 | 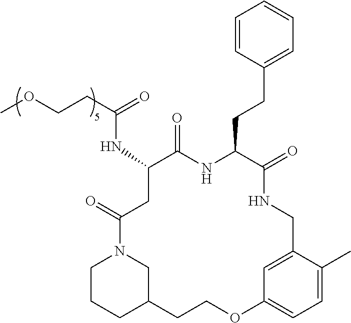 | N-((9S,12S)-5⁴-methyl- 8,11,14-trioxo-9-phenethyl- 4-oxa-7,10-diaza-1(3,1)- piperidina-5(1,3)- benzenacyclotetradecaphane- 12-yl)-2,5,8,11,14- pentaoxaheptadecan-17- amide | 768.4 C₄₁H₆₀N₄O₁₀ | 769.6 |
| 284 | 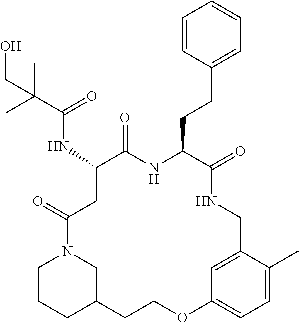 [SMILES: Cc1ccc2cc1CNC(=O)[C@H](CCc1ccccc1)NC(=O)[C@@H](NC(=O)C(C)(C)CO)CC(=O)N1CCCC(CCO2)C1] | 3-hydroxy-2,2-dimethyl- N-((9S,12S)-5⁴- methyl-8,11,14-trioxo- 9-phenethyl-4-oxa-7,10- diaza-1(3,1)-piperidina-5(1,3)- benzenacyclotetradecaphane- 12-yl)propanamide | 606.3 C₃₄H₄₆N₄O₆ | 607.5 |
| 285 | 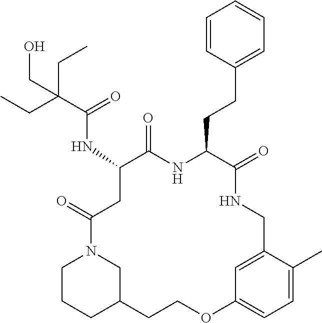 [SMILES: CCC(CC)(CO)C(=O)N[C@H]1CC(=O)N2CCCC(CCOc3ccc(C)c(c3)CNC(=O)[C@H](CCc3ccccc3)NC1=O)C2] | 2-ethyl-2-(hydroxymethyl)- N-((9S,12S)-5⁴-methyl- 8,11,14-trioxo-9-phenethyl- 4-oxa-7,10-diaza-1(3,1)- piperidina-5(1,3)- benzenacyclotetradecaphane- 12-yl)butanamide | 634.4 C₃₆H₅₀N₄O₆ | 635.5 |
| 286 | 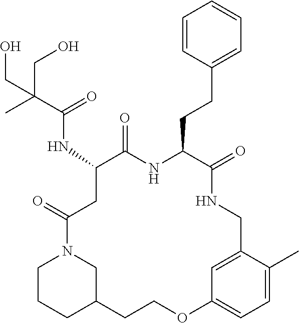 [SMILES: Cc1ccc2cc1CNC(=O)[C@H](CCc1ccccc1)NC(=O)[C@@H](NC(=O)C(C)(CO)CO)CC(=O)N1CCCC(CCO2)C1] | 3-hydroxy-2-(hydroxymethyl)- 2-methyl-N-((9S,12S)- 5⁴-methyl-8,11,14-trioxo-9- phenethyl-4-oxa-7,10-diaza- 1(3,1)-piperidina-5(1,3)- benzenacyclotetradecaphane- 12-yl)propanamide | 622.3 C₃₄H₄₆N₄O₇ | 623.5 |
| 287 | 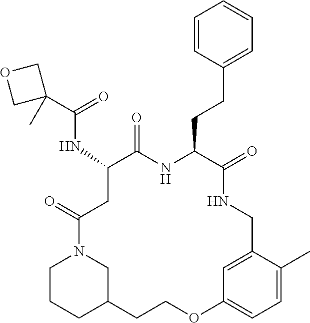 [SMILES: Cc1ccc2cc1CNC(=O)[C@H](CCc1ccccc1)NC(=O)[C@@H](NC(=O)C1(C)COC1)CC(=O)N1CCCC(CCO2)C1] | 3-methyl-N-((9S,12S)- 5⁴-methyl-8,11,14-trioxo- 9-phenethyl-4-oxa-7,10-diaza- 1(3,1)-piperidina-5(1,3)- benzenacyclotetradecaphane- 12-yl)oxetane-3-carboxamide | 604.3 C₃₄H₄₄N₄O₆ | 605.7 |
| 288 | 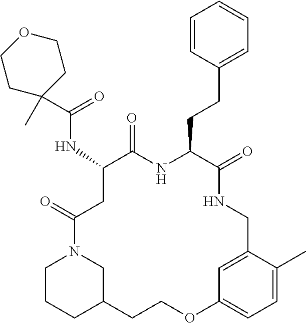 [SMILES: Cc1ccc2cc1CNC(=O)[C@H](CCc1ccccc1)NC(=O)[C@@H](NC(=O)C1(C)CCOCC1)CC(=O)N1CCCC(CCO2)C1] | 4-methyl-N-((9S,12S)- 5⁴-methyl-8,11,14-trioxo- 9-phenethyl-4-oxa-7,10-diaza- 1(3,1)-piperidina-5(1,3)- benzenacyclotetradecaphane- 12-yl)tetrahydro-2H-pyran- 4-carboxamide | 632.4 C₃₆H₄₈N₄O₆ | 633.5 |
| 323 | 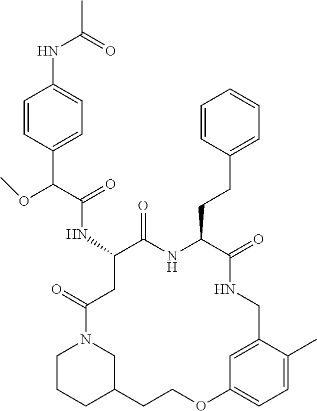 | 2-(4-acetamidophenyl)- 2-methoxy-N-((9S,12S)-5⁴- methyl-8,11,14-trioxo-9- phenethyl-4-oxa-7,10-diaza- 1(3,1)-piperidina-5(1,3)- benzenacyclotetradecaphane- 12-yl)acetamide | 711.4 C₄₀H₄₉N₅O₇ | 712.4 |
| 324 | 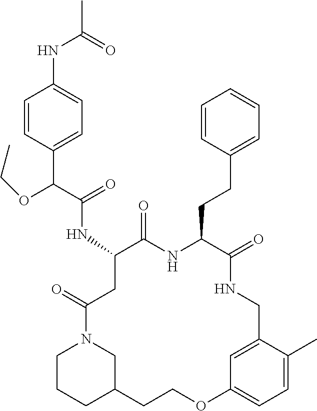 [SMILES: CCOC(C(=O)N[C@H]1CC(=O)N2CCCC(CCOc3ccc(C)c(c3)CNC(=O)[C@H](CCc3ccccc3)NC1=O)C2)c1ccc(NC(C)=O)cc1] | 2-(4-acetamidophenyl)-2- ethoxy-N-((9S,12S)-5⁴- methyl-8,11,14-trioxo-9- phenethyl-4-oxa-7,10-diaza- 1(3,1)-piperidina-5(1,3)- benzenacyclotetradecaphane- 12-yl)acetamide | 725.4 C₄₁H₅₁N₅O₇ | 726.4 |
| 327 | 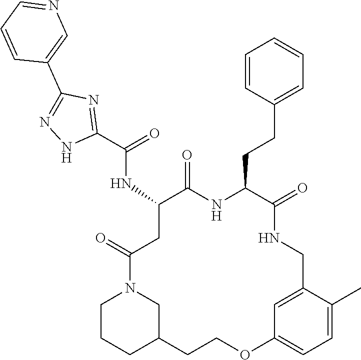 [SMILES: Cc1ccc2cc1CNC(=O)[C@H](CCc1ccccc1)NC(=O)[C@@H](NC(=O)c1nc(-c3cccnc3)n[nH]1)CC(=O)N1CCCC(CCO2)C1] | N-((9S,12R)-5⁴-methyl- 8,11,14-trioxo-9-phenethyl- 4-oxa-7,10-diaza-1(3,1)- piperidina-5(1,3)- benzenacyclotetradecaphane- 12-yl)-2-(3-(pyridin-3- yl)-1H-1,2,4-triazol-5- yl)acetamide | 692.3 C₃₈H₄₄N₈O₅ | 693.4 |
| 328 | 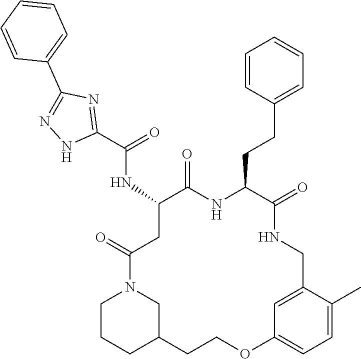 [SMILES: Cc1ccc2cc1CNC(=O)[C@H](CCc1ccccc1)NC(=O)[C@@H](NC(=O)c1nc(-c3ccccc3)n[nH]1)CC(=O)N1CCCC(CCO2)C1] | N-((9S,12S)-5⁴-methyl- 8,11,14-trioxo-9-phenethyl- 4-oxa-7,10-diaza-1(3,1)- piperidina-5(1,3)- benzenacyclotetradecaphane- 12-yl)-2-(3-phenyl-1H- 1,2,4-triazol-5-yl)acetamide | 691.3 C₃₉H₄₅N₇O₅ | 692.4 |
| 329 | 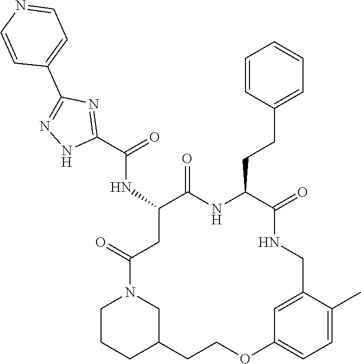 [SMILES: Cc1ccc2cc1CNC(=O)[C@H](CCc1ccccc1)NC(=O)[C@@H](NC(=O)c1nc(-c3ccncc3)n[nH]1)CC(=O)N1CCCC(CCO2)C1] | N-((9S,12S)-5⁴-methyl- 8,11,14-trioxo-9-phenethyl- 4-oxa-7,10-diaza-1(3,1)- piperidina-5(1,3)- benzenacyclotetradecaphane- 12-yl)-2-(3-(pyridin-4- yl)-1H-1,2,4-triazol-5- yl)acetamide | 692.3 C₃₈H₄₄N₈O₅ | 693.3 |
| 330 | 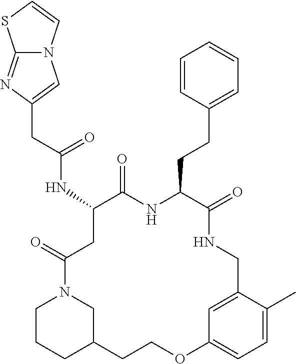 [SMILES: Cc1ccc2cc1CNC(=O)[C@H](CCc1ccccc1)NC(=O)[C@@H](NC(=O)Cc1cn3ccsc3n1)CC(=O)N1CCCC(CCO2)C1] | 2-(imidazo[2,1-b]thiazol-6- yl)-N-((9S,12S)-5⁴- methyl-8,11,14-trioxo-9- phenethyl-4-oxa-7,10-diaza- 1(3,1)-piperidina-5(1,3)- benzenacyclotetradecaphane- 12-yl)acetamide | 670.3 C₃₆H₄₂N₆O₅S | 671.3 |
| 331 | 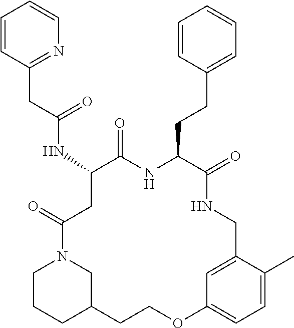 [SMILES: Cc1ccc2cc1CNC(=O)[C@H](CCc1ccccc1)NC(=O)[C@@H](NC(=O)Cc1ccccn1)CC(=O)N1CCCC(CCO2)C1] | N-((9S,12S)-5⁴-methyl- 8,11,14-trioxo-9-phenethyl- 4-oxa-7,10-diaza-1(3,1)- piperidina-5(1,3)- benzenacyclotetradecaphane- 12-yl)-2-(pyridin-2- yl)acetamide | 625.3 C₃₆H₄₃N₅O₅ | 507.4 |
| 332 | 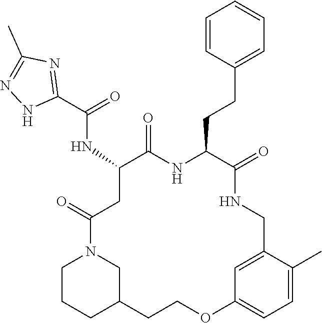 [SMILES: Cc1n[nH]c(C(=O)N[C@H]2CC(=O)N3CCCC(CCOc4ccc(C)c(c4)CNC(=O)[C@H](CCc4ccccc4)NC2=O)C3)n1] | 2-(3-methyl-1H-1,2,4- triazol-5-yl)-N-((9S,12S)-5⁴- methyl-8,11,14-trioxo-9- phenethyl-4-oxa-7,10-diaza- 1(3,1)-piperidina-5(1,3)- benzenacyclotetradecaphane- 12-yl)acetamide | 629.3 C₃₄H₄₃N₇O₅ | 630.4 |
| 333 | 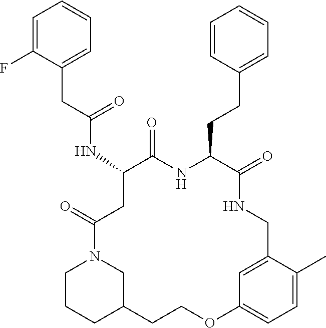 [SMILES: Cc1ccc2cc1CNC(=O)[C@H](CCc1ccccc1)NC(=O)[C@@H](NC(=O)Cc1ccccc1F)CC(=O)N1CCCC(CCO2)C1] | 2-(2-fluorophenyl)-N- ((9S,12S)-5⁴-methyl-8,11,14- trioxo-9-phenethyl-4-oxa-7,10- diaza-1(3,1)-piperidina-5(1,3)- benzenacyclotetradecaphane- 12-yl)acetamide | 642.3 C₃₇H₄₃FN₄O₅ | 643.4 |
| 334 | 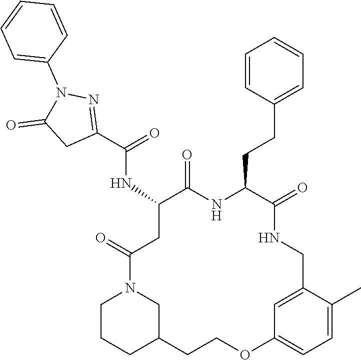 [SMILES: Cc1ccc2cc1CNC(=O)[C@H](CCc1ccccc1)NC(=O)[C@@H](NC(=O)C1=NN(c3ccccc3)C(=O)C1)CC(=O)N1CCCC(CCO2)C1] | N-((9S,12S)-5⁴-methyl- 8,11,14-trioxo-9-phenethyl- 4-oxa-7,10-diaza-1(3,1)- piperidina-5(1,3)- benzenacyclotetradecaphane- 12-yl)-2-(5-oxo-1-phenyl- 4,5-dihydro-1H-pyrazol-3- yl)acetamide | 706.3 C₄₀H₄₆N₆O₆ | 707.4 |
| 335 | 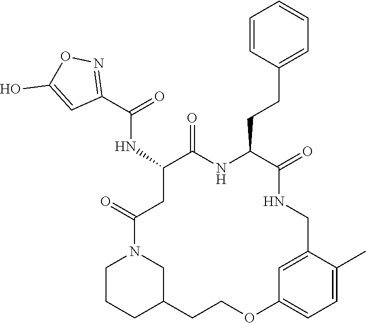 [SMILES: Cc1ccc2cc1CNC(=O)[C@H](CCc1ccccc1)NC(=O)[C@@H](NC(=O)c1cc(O)on1)CC(=O)N1CCCC(CCO2)C1] | 2-(5-hydroxyisoxazol- 3-yl)-N-((9S,12S)- 5⁴-methyl-8,11,14-trioxo- 9-phenethyl-4-oxa-7,10- diaza-1(3,1)-piperidina-5(1,3)- benzenacyclotetradecaphane- 12-yl)acetamide | 631.3 C₃₄H₄₁N₅O₇ | 632.4 |
| 341 | 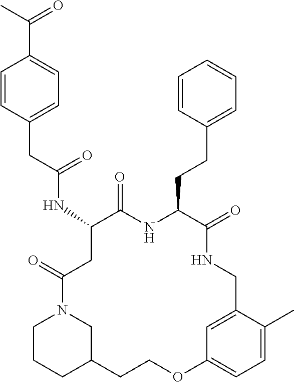 [SMILES: CC(=O)c1ccc(CC(=O)N[C@H]2CC(=O)N3CCCC(CCOc4ccc(C)c(c4)CNC(=O)[C@H](CCc4ccccc4)NC2=O)C3)cc1] | 2-(4-acetylphenyl)-N- ((9S,12S)-54- methyl-8,11,14-trioxo- 9-phenethyl-4-oxa-7,10- diaza-1(3,1)-piperidina-5(1,3)- benzenacyclotetradecaphane- 12-yl)acetamide | 666.3 C₃₉H₄₆N₄O₆ | 667.4 |
| 369 | 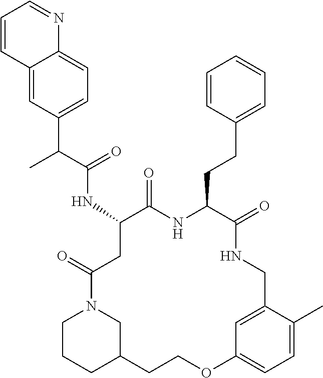 [SMILES: Cc1ccc2cc1CNC(=O)[C@H](CCc1ccccc1)NC(=O)[C@@H](NC(=O)C(C)c1ccc3ncccc3c1)CC(=O)N1CCCC(CCO2)C1] | N-((9S,12S)-5⁴-methyl- 8,11,14-trioxo-9-phenethyl- 4-oxa-7,10-diaza-1(3,1)- piperidina-5(1,3)- benzenacyclotetradecaphane- 12-yl)-2-(quinolin-6- yl)propanamide | 689.4 C₄₁H₄₇N₅O₅ | 690.4 |
| 373 | 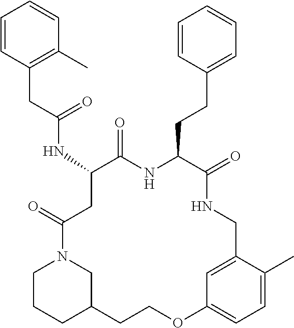 [SMILES: Cc1ccc2cc1CNC(=O)[C@H](CCc1ccccc1)NC(=O)[C@@H](NC(=O)Cc1ccccc1C)CC(=O)N1CCCC(CCO2)C1] | N-((9S,12S)-5⁴-methyl- 8,11,14-trioxo-9-phenethyl- 4-oxa-7,10-diaza-1(3,1)- piperidina-5(1,3)- benzenacyclotetradecaphane- 12-yl)-2-(o-tolyl)acetamide | 638.3 C₃₈H₄₆N₄O₅ | 639.4 |

### Example A-4: Preparation of Macrocyclic Compounds 34, 35, 36, 37, 38, 39, 43, 44, 45, and 364

### (9S,12S)-5⁴-methyl-9-phenethyl-12-(pyrimidin-2-ylamino)-4-oxa-7,10-diaza-1(3,1)

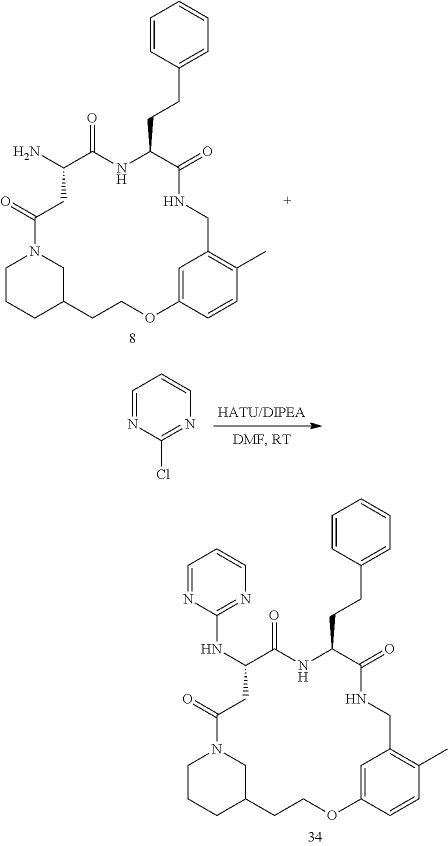 [SMILES: Cc1ccc2cc1CNC(=O)[C@H](CCc1ccccc1)NC(=O)[C@@H](N)CC(=O)N1CCCC(CCO2)C1 ; Clc1ncccn1 ; Cc1ccc2cc1CNC(=O)[C@H](CCc1ccccc1)NC(=O)[C@@H](Nc1ncccn1)CC(=O)N1CCCC(CCO2)C1]

20 mg amine 8, 9 mg 2-chloropyrimidine and 25 mg K₃PO₄ in 0.5 ml DMF were heated to 100° C. over night. Diluted with some methanol, filtered and purified via HPLC.

Formula: C₃₃H₄₀N₆O₄, exact mass: 584.3, found: 585.3 [M+H]⁺

### (9S,12S)-5⁴-methyl-12-(oxetan-3-ylamino)-9-phenethyl-4-oxa-7,10-diaza-1(3,1)-piperidina-5(1,3)-benzenacyclotetradecaphane-8,11,14-trione (35)

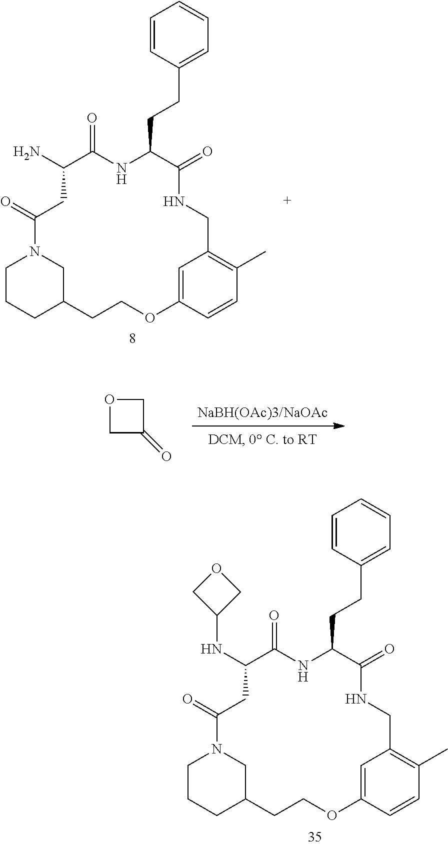 [SMILES: Cc1ccc2cc1CNC(=O)[C@H](CCc1ccccc1)NC(=O)[C@@H](N)CC(=O)N1CCCC(CCO2)C1 ; O=C1COC1 ; Cc1ccc2cc1CNC(=O)[C@H](CCc1ccccc1)NC(=O)[C@@H](NC1COC1)CC(=O)N1CCCC(CCO2)C1]

20 mg amine 8 and 4 mg 3-oxetanone in DCM were cooled in an ice bath. Sodium acetate (0.5 eq.) and 3 eq. sodium triacetoxyborohydride were added and the reaction mixture was allowed to warm to room temperature overnight. Additional 4.3 mg ketone and 17 mg borohydride were added and stirring was continued for another 3h when some methanol was added and the reaction mixture was purified via HPLC.

Formula: C₃₂H₄₂N₄O₅, exact mass: 562.3, found: 563.5 [M+H]⁺

### (9S,12S)-5⁴-methyl-9-phenethyl-12-(pyridin-2-ylamino)-4-oxa-7,10-diaza-1(3,1)-piperidina-5(1,3)-benzenacyclotetradecaphane-8,11,14-trione (36)

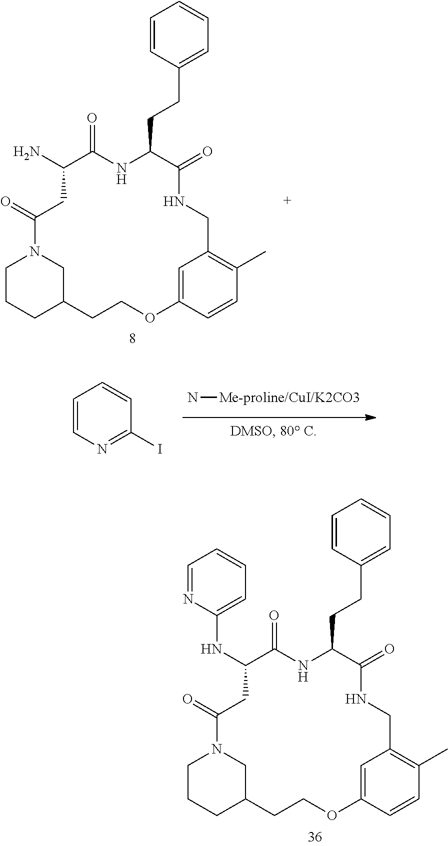

20 mg amine 8, 6.4 mg 2-iodopyridine, 5.2 mg N-Me-proline, 3.8 mg CuI and 11.1 mg K₂CO₃ in 500 μl DMSO were heated to 80° C. for 24 h. The mixture was diluted with some methanol, filtered and purified via HPLC.

Formula: C₃₄H₄₁N₅O₄, exact mass: 583.3, found: 584.3 [M+H]⁺

### N-((9S,12S)-5⁴-methyl-8,11,14-trioxo-9-phenethyl-4-oxa-7,10-diaza-1(3,1)-piperidina-5(1,3)-benzenacyclotetradecaphane-12-yl)methanesulfonamide (37)

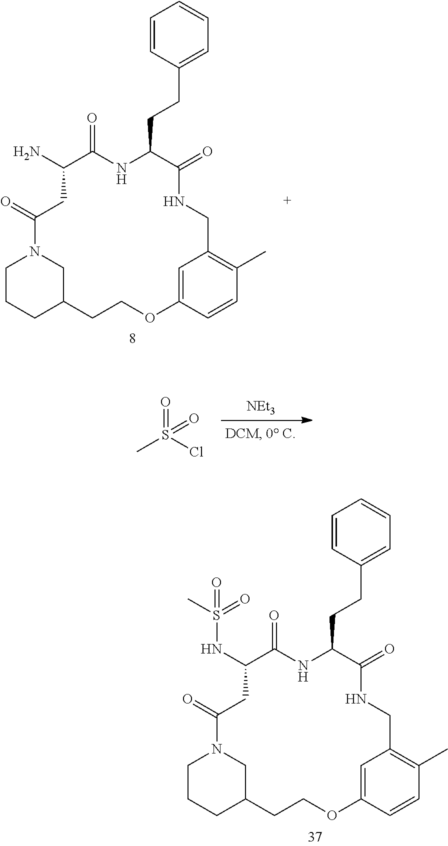

Compound 37 was synthesized according to general procedure E with 20 mg amine 8. Product was purified by HPLC.

Formula: C₃₀H₄₀N₄O₆S, exact mass: 584.3, found: 585.4 [M+H]⁺

### 1-ethyl-3-((9S,12S)-5⁴-methyl-8,11,14-trioxo-9-phenethyl-4-oxa-7,10-diaza-1(3,1)-piperidina-5(1,3)-benzenacyclotetradecaphane-12-yl)urea (38)

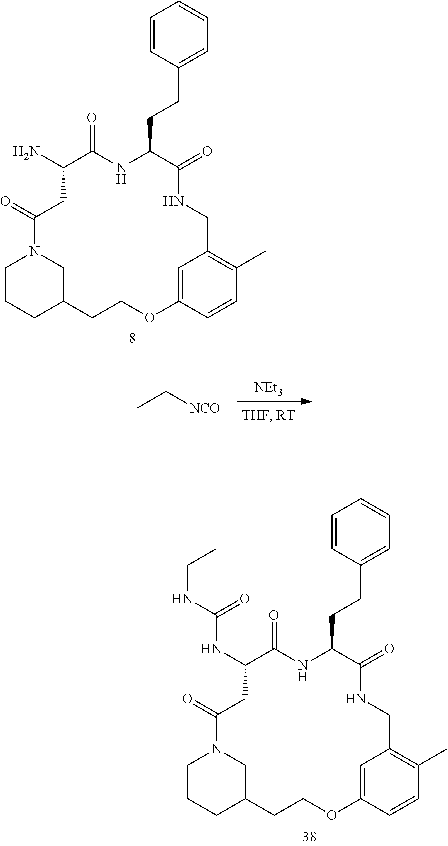

Compound 38 was synthesized according to general procedure F with 20 mg amine 8 in 0.5 ml THF.

Formula: C₃₂H₄₃N₅O₅, exact mass: 577.3, found: 578.4 [M+H]⁺

### ethyl ((9S,12S)-5⁴-methyl-8,11,14-trioxo-9-phenethyl-4-oxa-7,10-diaza-1(3,1)-piperidina-5(1,3)-benzenacyclotetradecaphane-12-yl)carbamate (39)

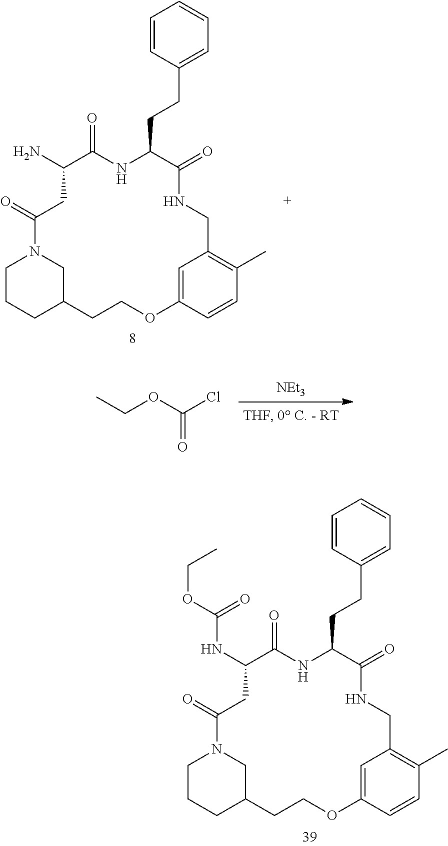

Compound 39 was synthesized according to general procedure G with 20 mg amine 8 in 0.5 ml THF.

Formula: C₃₂H₄₂N₄O₆, exact mass: 578.3, found: 579.4 [M+H]⁺

### (9S,12S)-12-(2,5-dioxopyrrolidin-1-yl)-5⁴-methyl-9-phenethyl-4-oxa-7,10-diaza-1(3,1)-piperidina-5(1,3)-benzenacyclotetradecaphane-8,11,14-trione (43)

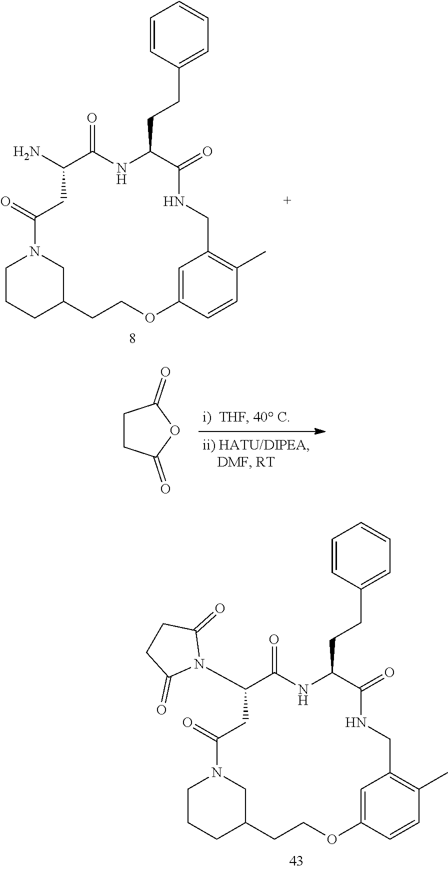 [SMILES: Cc1ccc2cc1CNC(=O)[C@H](CCc1ccccc1)NC(=O)[C@@H](N)CC(=O)N1CCCC(CCO2)C1 ; O=C1CCC(=O)O1 ; Cc1ccc2cc1CNC(=O)[C@H](CCc1ccccc1)NC(=O)[C@@H](N1C(=O)CCC1=O)CC(=O)N1CCCC(CCO2)C1]

10 mg amine 8 and 2.0 mg succinic anhydride in 0.3 ml THF were stirred at 40° C. overnight. Volatiles were removed under reduced pressure and the residue dissolved in 300 μl DMF. 8.4 mg HATU and 20.9 μl DIPEA in additional 300 μl DMF were added at RT. After complete reaction some methanol was added and purification was achieved by HPLC.

Formula: C₃₃H₄₀N₄O₆, exact mass: 588.3, found: 589.4 [M+H]⁺

### N-((9S,12S)-5⁴-methyl-8,11,14-trioxo-9-phenethyl-4-oxa-7,10-diaza-1(3,1)-piperidina-5(1,3)-benzenacyclotetradecaphane-12-yl)-3-phenyloropane-1-sulfonamide (44)

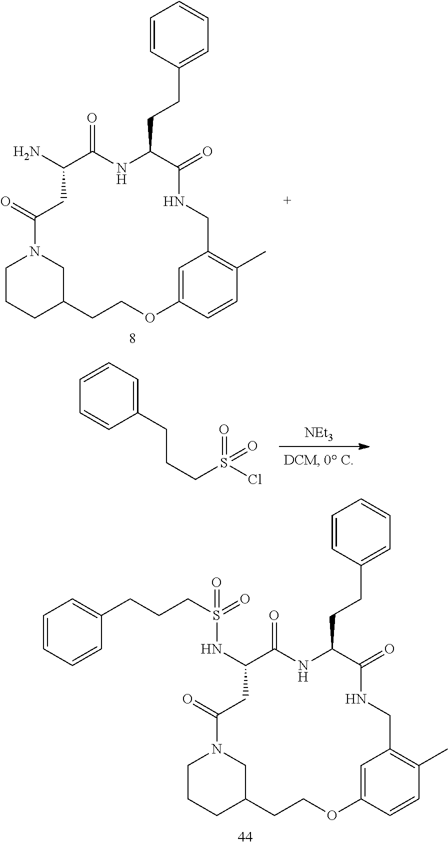 [SMILES: Cc1ccc2cc1CNC(=O)[C@H](CCc1ccccc1)NC(=O)[C@@H](N)CC(=O)N1CCCC(CCO2)C1 ; O=S(=O)(Cl)CCCc1ccccc1 ; Cc1ccc2cc1CNC(=O)[C@H](CCc1ccccc1)NC(=O)[C@@H](NS(=O)(=O)CCCc1ccccc1)CC(=O)N1CCCC(CCO2)C1 ; "NEt3"]

Compound 44 was synthesized according to general procedure E with 20 mg amine 8. Product was purified by HPLC.

Formula: C₃₈H₄₈N₄O₆S, exact mass: 688.3, found: 689.5 [M+H]⁺

### (9S,12S)-12-((1H-benzo[d]imidazol-2-yl)amino)-5⁴-methyl-9-phenethyl-4-oxa-7,10-diaza-1(3,1)-piperidina-5(1,3)-benzenacyclotetradecaphane-8,11,14-trione (45)

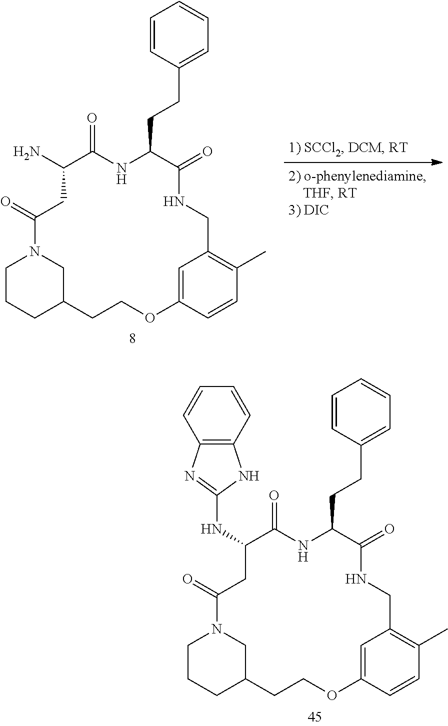 [SMILES: Cc1ccc2cc1CNC(=O)[C@H](CCc1ccccc1)NC(=O)[C@@H](N)CC(=O)N1CCCC(CCO2)C1 ; Cc1ccc2cc1CNC(=O)[C@H](CCc1ccccc1)NC(=O)[C@@H](Nc1nc3ccccc3[nH]1)CC(=O)N1CCCC(CCO2)C1]

To 20 mg amine 8 in 1 ml DCM 30.5 μl thiophosgene were added. 1 ml saturated NaHCO₃ solution was added and the mixture was stirred for until starting material was consumed. The organic phase was separated, dried and volatiles were removed under reduced pressure. The residue and 5.2 mg o-phenylenediamine were dissolved in 0.5 ml THF and stirred overnight to give the intermediate thiourea. 5.6 mg N,N′-diisopropylcarbodiimid were added and the mixture was kept at 55° C. until reaction was almost complete. The mixture was diluted with some methanol and purified by HPLC.

Formula: C₃₆H₄₂N₆O₄, exact mass: 622.3, found: 623.5 [M+H]⁺

### N-((9S,12S)-8,11,14-trioxo-9-phenethyl-4-oxa-7,10-diaza-1(3,1)-piperidina-5(1,3)-benzenacyclotetradecaphane-12-yl)acetamide (364)

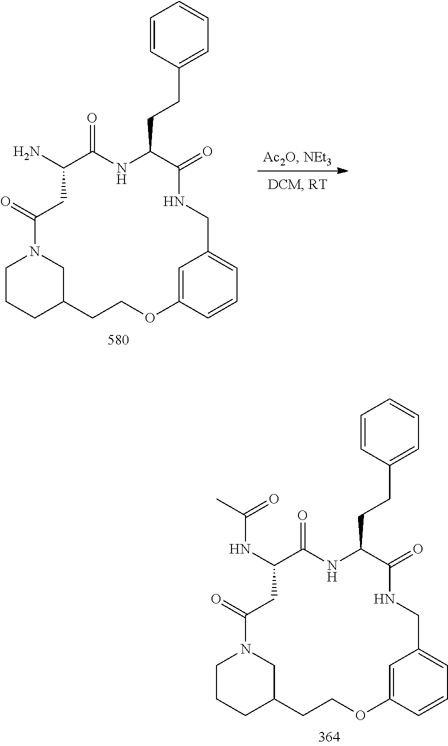

Compound 580 was made in analogy to compound 8 utilizing tert-butyl-3-(2-(3-aminomethyl(phenoxy)ethyl)piperidine-1-carboxylate 579 instead of amine 4.

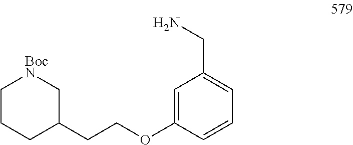 [SMILES: CC(C)(C)OC(=O)N1CCCC(CCOc2cccc(CN)c2)C1]

Formula: C₃₀H₃₈N₄O₅, exact mass: 534.3, found: 535.5 [M+H]⁺

### Example A-5: Preparation of Macrocyclic Compound 51

### 2-methyl-5-(2-(piperidin-3-yl)ethoxy)benzonitrile (46)

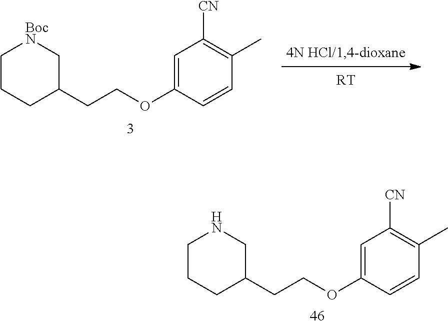 [SMILES: Cc1ccc(OCCC2CCCN(C(=O)OC(C)(C)C)C2)cc1C#N ; "RT" ; "4N HCl/1,4-dioxane" ; Cc1ccc(OCCC2CCCNC2)cc1C#N]

Compound 46 was prepared from 390 mg Boc-protected amine 3 according to general procedure D with 4 N HCl in 1,4-dioxane at RT. Raw product was coevaporated twice with methanol and use without further purification.

Formula: C₁₅H₂₀N₂O, exact mass: 244.2, found: 245.2 [M+H]⁺

### tert-butyl 4-(3-(2-(3-cyano-4-methylphenoxy)ethyl)piperidin-1-yl)-4-oxobutanoate (47)

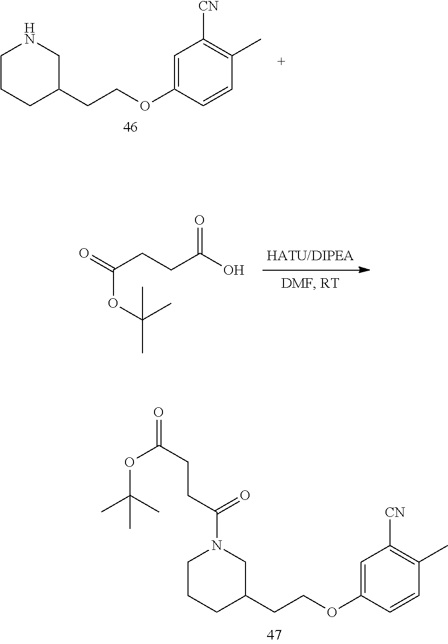

Synthesis according to general procedure B with 1.13 mmol amine 4 and 296 mg mono-tert.-butyl succinate. Purification was achieved by normal phase flash chromatography.

Formula: C₂₃H₃₂N₂O₄, exact mass: 400.2, found: 401.4 [M+H]⁺

### tert-butyl 4-(3-(2-(3-(aminomethyl)-4-methylphenoxy)ethyl)piperidin-1-yl)-4-oxobutanoate (48)

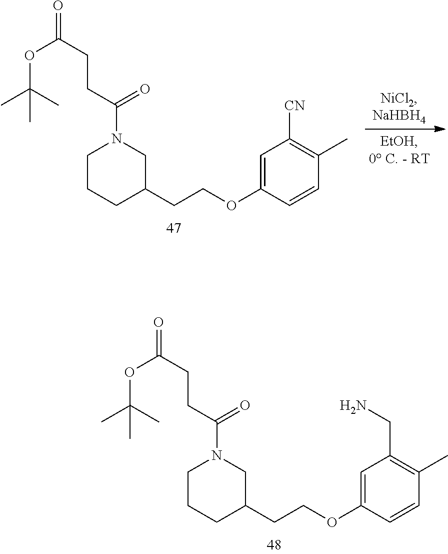

142 mg nitrile 47 and 91 mg NiCl₂ were stirred in 7 ml ethanol at 0° C. 54 mg NaBH₄ were added and the mixture was allowed to come to room temperature. After 1 h each same amounts as before of NaBH₄ and NiCl₂ were added. After 1 h the mixture was filtered over Celite. Filtrate was concentrated under reduced pressure, distributed between water and ethyl acetate. The organic phase was separated and the aqueous phase extracted several times with ethyl acetate. Combined organic phases were dried over MgSO₄ and solvent was removed under reduce pressure. The crude was used without further purification.

Formula: C₂₃H₃₆N₂O₄, exact mass: 404.3, found: 405.1 [M+H]⁺

### tert-butyl 4-(3-(2-(3-((2-((tert-butoxycarbonyl)amino)-4-(pyridin-3-yl)butanamido)methyl)-4-methylphenoxy)ethyl)piperidin-1-yl)-4-oxobutanoate (49)

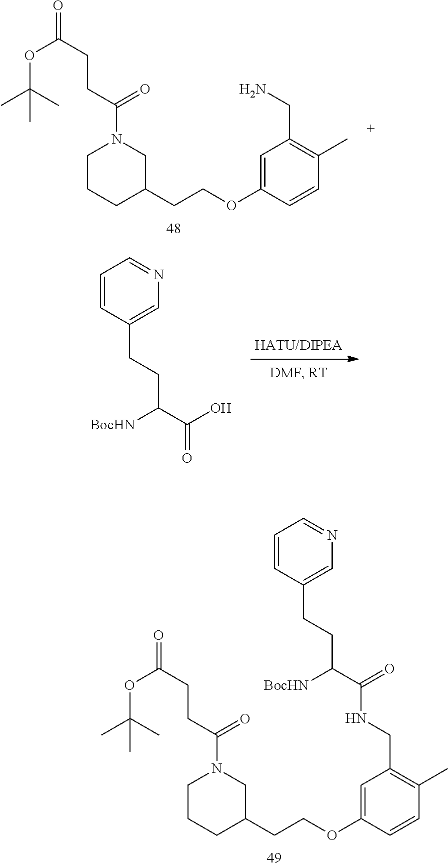

Synthesis according to general procedure B with 94 mg amine 48 and 50 mg 2-((tert-butoxycarbonyl)amino)-4-(pyridin-3-yl)butanoic acid. Purification was achieved by normal phase flash chromatography

Formula: C₃₇H₅₄N₄O₇, exact mass: 666.4, found: 667.4 [M+H]⁺

### 4-(3-(2-(3-((2-amino-4-(pyridin-3-yl)butanamido)methyl)-4-methylphenoxy)ethyl) piperidin-1-yl)-4-oxobutanoic acid (50)

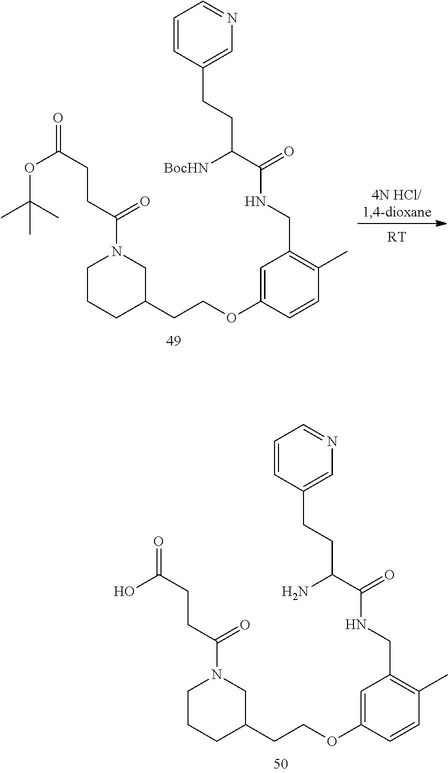

Protecting groups were cleaved according to general procedure D stirring compounds in 4 N HCl/1,4-dioxane at room temperature. After deprotection was complete solvent was removed and the crude dissolved in acetonitrile and concentrated twice. Product used without further purification.

Formula: C₂₈H₃₈N₄O₅, exact mass: 510.3, found: 511.3 [M+H]⁺

### 5⁴-methyl-9-(2-(pyridin-3-yl)ethyl)-4-oxa-7,10-diaza-1(3,1)-piperidina-5(1,3)-benzenacyclotetradecaphane-8,11,14-trione (51)

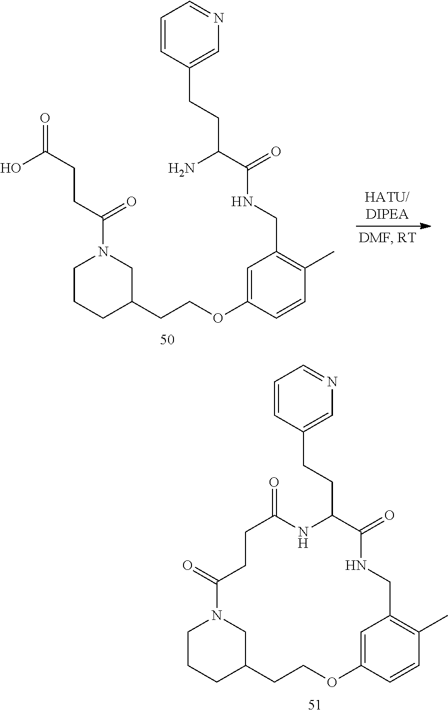 [SMILES: Cc1ccc(OCCC2CCCN(C(=O)CCC(=O)O)C2)cc1CNC(=O)C(N)CCc1cccnc1 ; Cc1ccc2cc1CNC(=O)C(CCc1cccnc1)NC(=O)CCC(=O)N1CCCC(CCO2)C1]

Macrocyclization was achieved according to general procedure A with 0.179 mmol amino acid 50. Reaction mixtures were diluted with some methanol and purified via HPLC.

Formula: C₂₈H₃₆N₄O₄, exact mass: 492.3, found: 493.3 [M+H]⁺

### Example A-6: Preparation of Macrocyclic Compound 59

### tert-butyl 3-(2-(5-bromo-2,4-dimethylphenoxy)ethyl)piperidine-1-carboxylate (52)

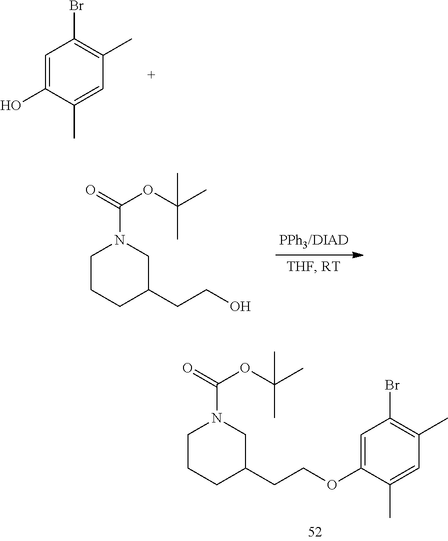

855 mg tert-butyl 3-(2-hydroxyethyl)piperidine-1-carboxylate, 500 mg 5-bromo-2,4-dimethylphenol and 978 mg PPh₃ were dissolved in 12 ml THF. 734 μl diisopropyl azodicarboxylate in 3 ml THF were added dropwise and the reaction was stirred for 2 h at room temperature. The reaction was diluted with ethyl acetate and washed with saturated NaHCO₃ solution and brine. After drying the organic phase over MgSO₄ and removing volatiles under reduced pressure the crude was purified by normal phase flash chromatography.

Formula: C₂₀H₃₀BrNO₃, exact mass: 411.1, found: 414.1 [M+H]⁺

### tert-butyl 3-(2-(5-cyano-2,4-dimethylphenoxy)ethyl)piperidine-1-carboxylate (53)

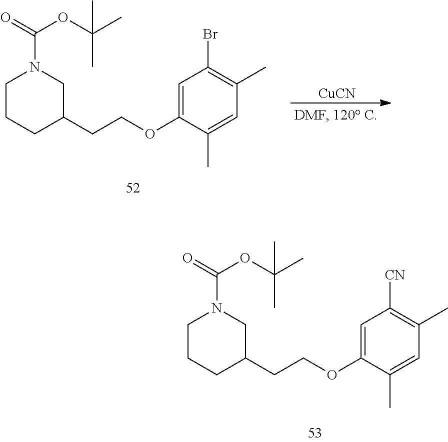

Bromide 52 (780 mg) and 340 mg CuCN in DMF were stirred at 120° C. for 4d. The mixture was diluted with ethyl acetate, washed with saturated NaHCO₃ solution and brine and dried over MgSO₄. Solvent was removed under reduced pressure and the residue was purified by normal phase column chromatography (silica, cyclohexane/ethyl acetate gradient).

Formula: C₂₁H₃₀N₂O₃, exact mass: 358.2, found: 358.3 [M+H]⁺

### tert-butyl 3-(2-(5-(aminomethyl)-2,4-dimethylphenoxy)ethyl)piperidine-1_-carboxylate (54)

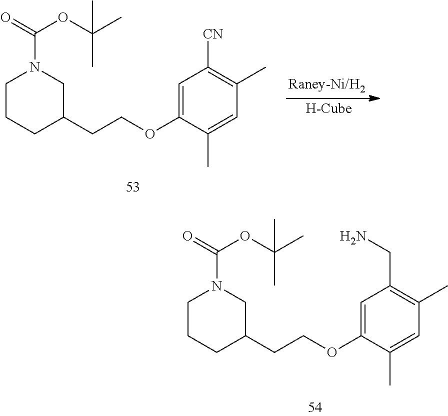 [SMILES: Cc1cc(C)c(OCCC2CCCN(C(=O)OC(C)(C)C)C2)cc1C#N ; Cc1cc(C)c(OCCC2CCCN(C(=O)OC(C)(C)C)C2)cc1CN]

Nitrile 53 (432 mg) in 40 ml EtOH were reduced according to general procedure H in 4 cycles at 50 bar, 70° C. and 0.5 ml/min. After removing volatiles the residue was used without further purification.

Formula: C₂₁H₃₄N₂O₃, exact mass: 362.3, found: 363.3 [M+H]⁺

### tert-butyl 3-(2-(5-(((S)-2-(((benzyloxy)carbonyl)amino)-4-phenylbutanamido)methyl)-2,4-dimethylphenoxy)ethyl)piperidine-1-carboxylate (55)

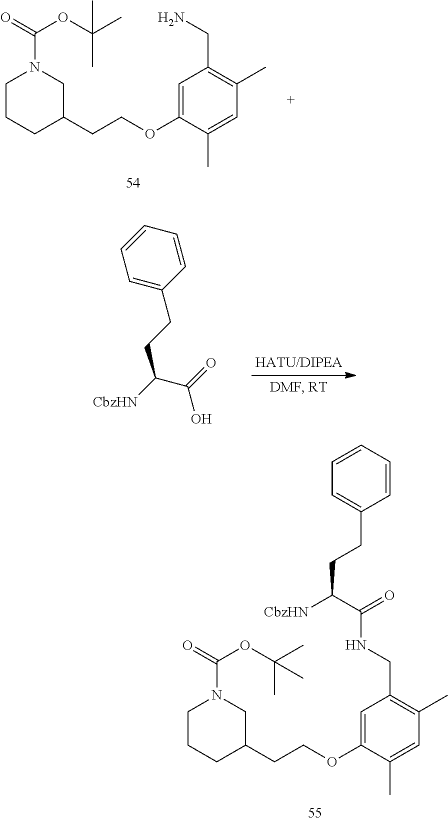 [SMILES: Cc1cc(C)c(OCCC2CCCN(C(=O)OC(C)(C)C)C2)cc1CN ; O=C(N[C@@H](CCc1ccccc1)C(=O)O)OCc1ccccc1 ; Cc1cc(C)c(OCCC2CCCN(C(=O)OC(C)(C)C)C2)cc1CNC(=O)[C@H](CCc1ccccc1)NC(=O)OCc1ccccc1]

Synthesis according to general procedure B with 1.21 mmol amine 54 and 493 mg (S)-2-(((benzyloxy)carbonyl)amino)-4-phenylbutanoic acid. Purification was achieved by normal phase flash chromatography.

Formula: C₃₉H₅₁N₃O₆, exact mass: 657.4, found: 658.3 [M+H]⁺

### tert-butyl 3-(2-(5-(((S)-2-amino-4-phenylbutanamido)methyl)-2,4-dimethylphenoxy) ethyl)piperidine-1-carboxylate (56)

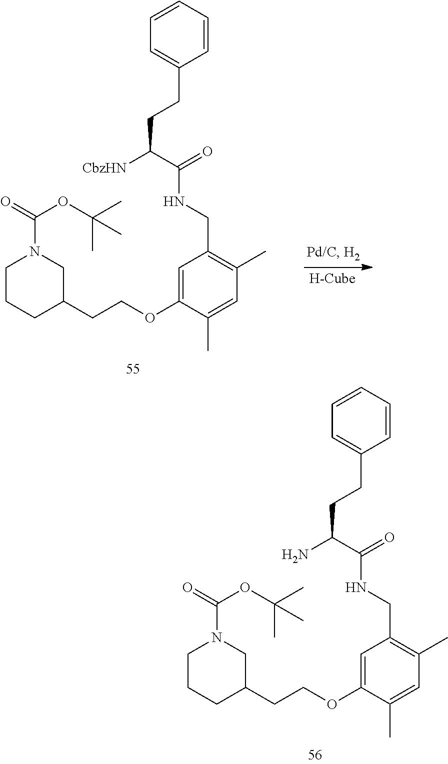 [SMILES: Cc1cc(C)c(OCCC2CCCN(C(=O)OC(C)(C)C)C2)cc1CNC(=O)[C@H](CCc1ccccc1)NC(=O)OCc1ccccc1 ; Cc1cc(C)c(OCCC2CCCN(C(=O)OC(C)(C)C)C2)cc1CNC(=O)[C@@H](N)CCc1ccccc1]

Synthesis according general procedure C with 695 mg protected amine in 80 ml ethanol/ethyl acetate (1:1) with 20 bar, 50° C. and 0.5 ml/min. After removing volatiles the crude was used for the next step.

Formula: C₃₁H₄₅N₃O₄, exact mass: 523.3, found: 524.4 [M+H]⁺

### tert-butyl 3-(2-(5-(((S)-2-(4-(tert-butoxy)-4-oxobutanamido)-4-phenylbutanamido)methyl)-2,4-dimethylphenoxy)ethyl)piperidine-1-carboxylate (57)

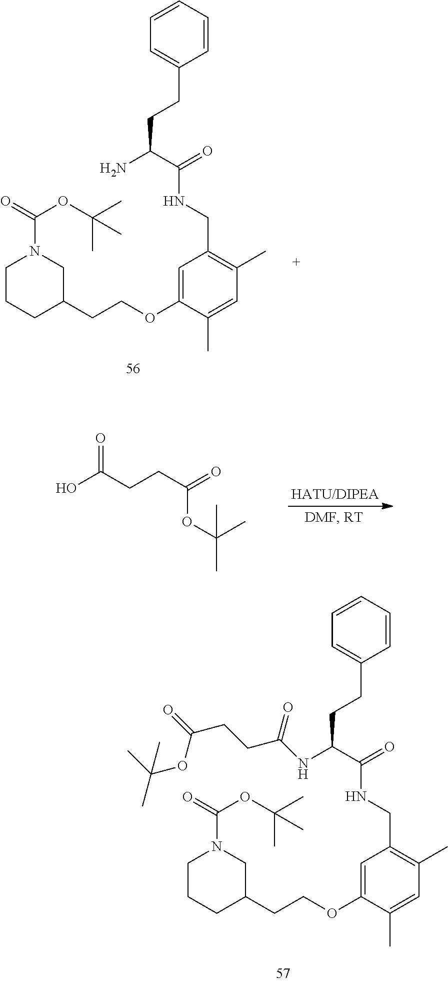 [SMILES: Cc1cc(C)c(OCCC2CCCN(C(=O)OC(C)(C)C)C2)cc1CNC(=O)[C@@H](N)CCc1ccccc1 ; CC(C)(C)OC(=O)CCC(=O)O ; Cc1cc(C)c(OCCC2CCCN(C(=O)OC(C)(C)C)C2)cc1CNC(=O)[C@H](CCc1ccccc1)NC(=O)CCC(=O)OC(C)(C)C]

Synthesis according to general procedure B with 200 mg amine 56 and 100 mg mono-tert.-butyl succinate. Purification was achieved by normal phase flash chromatography.

Formula: C₃₉H₅₇N₃O₇, exact mass: 679.4, found: 680.4 [M+H]⁺

### 4-(((2S)-1-((2,4-dimethyl-5-(2-(piperidin-3-yl)ethoxy)benzyl)amino)-1-oxo-4-phenylbutan-2-yl)amino)-4-oxobutanoic acid (58)

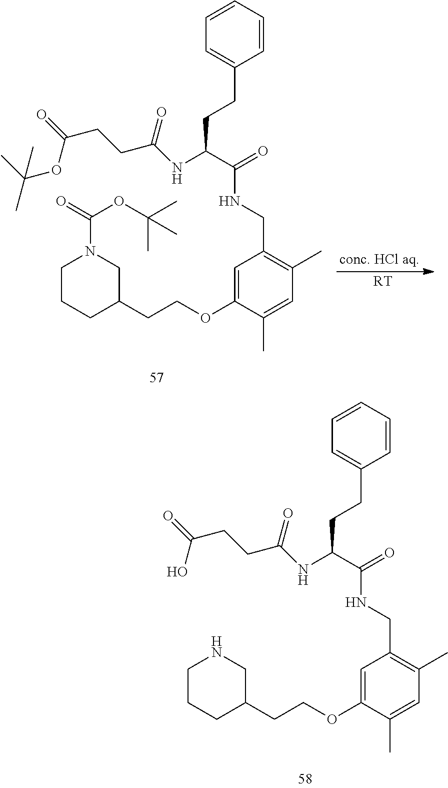 [SMILES: Cc1cc(C)c(OCCC2CCCN(C(=O)OC(C)(C)C)C2)cc1CNC(=O)[C@H](CCc1ccccc1)NC(=O)CCC(=O)OC(C)(C)C ; "RT" ; Cc1cc(C)c(OCCC2CCCNC2)cc1CNC(=O)[C@H](CCc1ccccc1)NC(=O)CCC(=O)O]

Both protecting groups were cleaved according to general procedure D in concentrated HCl in water at RT for 2h. Crude was coevaporated twice with acetonitrile and used without further purification.

Formula: C₃₀H₄₁N₃O₅, exact mass: 523.3, found: −[M+H]⁺

### (9R)-5⁴,5⁶-dimethyl-9-phenethyl-4-oxa-7,10-diaza-1(3,1)-piperidina-5(1,3)-benzenacyclotetradecaphane-8,11,14-trione (59)

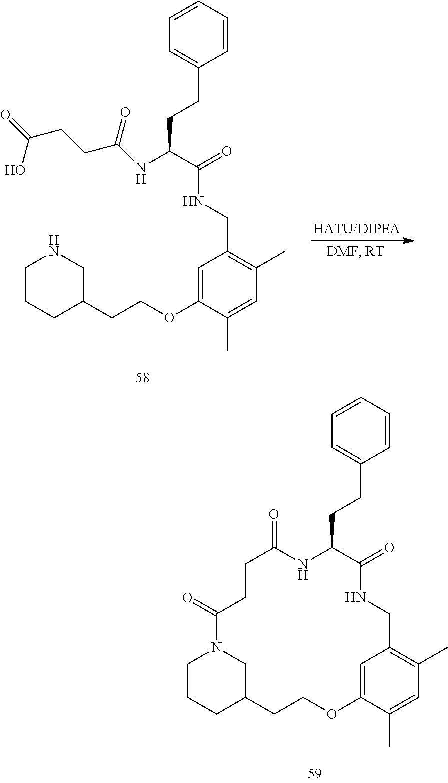 [SMILES: Cc1cc(C)c(OCCC2CCCNC2)cc1CNC(=O)[C@H](CCc1ccccc1)NC(=O)CCC(=O)O ; Cc1cc(C)c2cc1CNC(=O)[C@H](CCc1ccccc1)NC(=O)CCC(=O)N1CCCC(CCO2)C1]

Macrocyclization was achieved according to general procedure A with 0.447 mmol amino acid 58. Reaction mixtures were diluted with some methanol and purified via HPLC.

Formula: C₃₀H₃₉N₃O₄, exact mass: 505.3, found: 506.3 [M+H]⁺

### Example A-7: Preparation of Macrocyclic Compounds 77, 78, and 79

### (S)-tert-butyl 4-((1-(benzyloxy)-3-(1H-indol-3-yl)-1-oxopropan-2-yl)amino)-4-oxobutanoate (65)

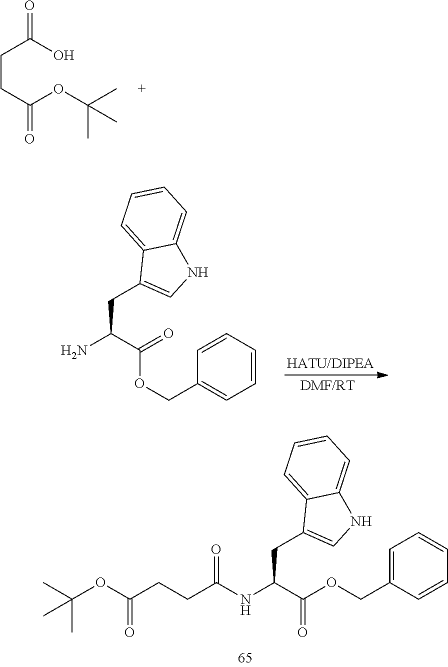 [SMILES: CC(C)(C)OC(=O)CCC(=O)O ; N[C@@H](Cc1c[nH]c2ccccc12)C(=O)OCc1ccccc1 ; CC(C)(C)OC(=O)CCC(=O)N[C@@H](Cc1c[nH]c2ccccc12)C(=O)OCc1ccccc1]

The amide bond was formed according to general procedure B with 1.02 g (S)-benzyl 2-amino-3-(1H-indol-3-yl)propanoate and 400 mg mono-tert.-butyl succinate. Purification was achieved by RP18 reversed phase flash chromatography.

Formula: C₂₆H₃₀N₂O₅, exact mass: 450.2, found: 451.3 [M+H]⁺

### (S)-tert-butyl 4-((1-(benzyloxy)-1-oxopropan-2-yl)amino)-4-oxobutanoate (66)

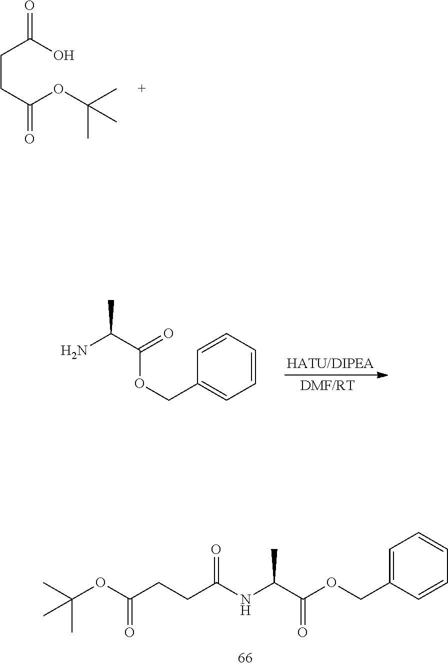 [SMILES: CC(C)(C)OC(=O)CCC(=O)O ; C[C@H](N)C(=O)OCc1ccccc1 ; C[C@H](NC(=O)CCC(=O)OC(C)(C)C)C(=O)OCc1ccccc1]

The amide bond was formed according to general procedure B with 744 g (S)-benzyl 2-aminopropanoate and 400 mg mono-tert.-butyl succinate. Purification was achieved by RP18 reversed phase flash chromatography.

Formula: C₁₈H₂₅NO₅, exact mass: 360.2, found: 361.2 [M+H]⁺

### (S)-tert-butyl 4-((1-(benzyloxy)-1-oxo-3-phenylpropan-2-yl)amino)-4-oxobutanoate (67)

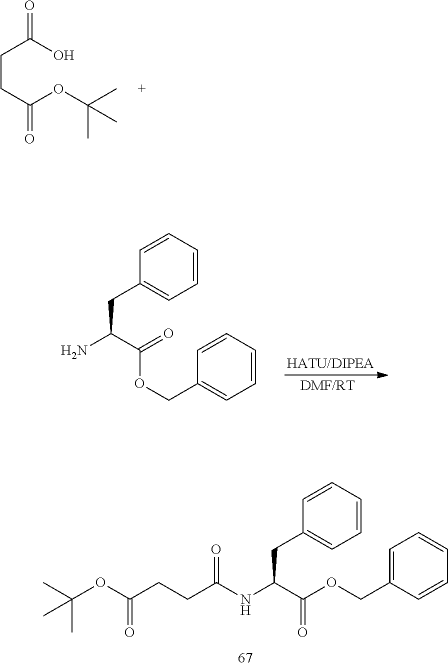 [SMILES: CC(C)(C)OC(=O)CCC(=O)O ; N[C@@H](Cc1ccccc1)C(=O)OCc1ccccc1 ; CC(C)(C)OC(=O)CCC(=O)N[C@@H](Cc1ccccc1)C(=O)OCc1ccccc1]

The amide bond was formed according to general procedure B with 1.01 g (S)-benzyl 2-amino-3-phenylpropanoate and 400 mg mono-tert.-butyl succinate. Purification was achieved by RP18 reversed phase flash chromatography.

Formula: C₂₄H₂₉NO₅, exact mass: 411.2, found: 412.3 [M+H]⁺

### (S)-2-(4-(tert-butoxy)-4-oxobutanamido)-3-(1H-indol-3-yl)propanoic acid (68)

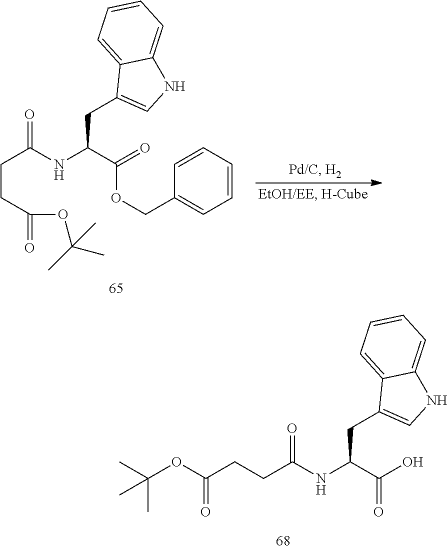 [SMILES: CC(C)(C)OC(=O)CCC(=O)N[C@@H](Cc1c[nH]c2ccccc12)C(=O)OCc1ccccc1 ; CC(C)(C)OC(=O)CCC(=O)N[C@@H](Cc1c[nH]c2ccccc12)C(=O)O]

Benzyl ester was cleaved according to general procedure C using Raney-Ni catalyst cartridge, 20 bar at 50° C. in a solvent mixture of ethanol/ethyl acetate (3:1). Product was purified by normal phase column chromatography (silica, cyclohexane/ethyl acetate gradient). Formula: C₁₉H₂₄N₂O₅, exact mass: 360.2, found: 361.2 [M+H]⁺

### (S)-2-(4-(tert-butoxy)-4-oxobutanamido)propanoic acid (69)

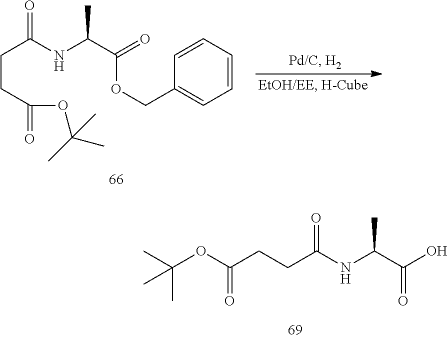 [SMILES: C[C@H](NC(=O)CCC(=O)OC(C)(C)C)C(=O)OCc1ccccc1 ; C[C@H](NC(=O)CCC(=O)OC(C)(C)C)C(=O)O]

Benzyl ester was cleaved according to general procedure C using Raney-Ni catalyst cartridge, 20 bar at 50° C. in a solvent mixture of ethanol/ethyl acetate (3:1). Product was purified by normal phase column chromatography (silica, cyclohexane/ethyl acetate gradient). Formula: C₁₁H₁₉NO₅, exact mass: 245.1, found: 246.2 [M+H]⁺

### (S)-2-(4-(tert-butoxy)-4-oxobutanamido)-3-Phenylpropanoic acid (70)

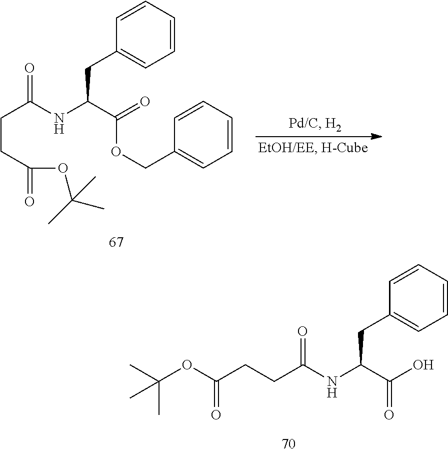 [SMILES: CC(C)(C)OC(=O)CCC(=O)N[C@@H](Cc1ccccc1)C(=O)OCc1ccccc1 ; CC(C)(C)OC(=O)CCC(=O)N[C@@H](Cc1ccccc1)C(=O)O]

Benzyl ester was cleaved according to general procedure C using Raney-Ni catalyst cartridge, 20 bar at 50° C. in a solvent mixture of ethanol/ethyl acetate (3:1). Product was purified by normal phase column chromatography (silica, cyclohexane/ethyl acetate gradient). Formula: C₁₇H₂₃NO₅, exact mass: 321.2, found: 322.2 [M+H]⁺

### tert-butyl 3-(2-(3-(((S)-2-(4-(tert-butoxy)-4-oxobutanamido)-3-(1H-indol-3-yl)propanamido)methyl)-4-methylphenoxy)ethyl)Piperidine-1-carboxylate (71)

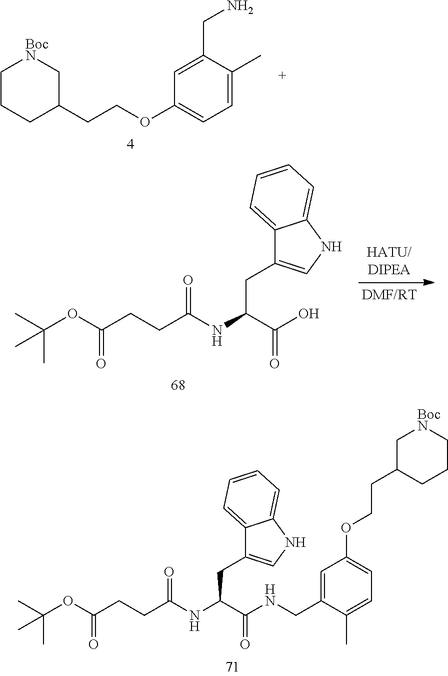 [SMILES: Cc1ccc(OCCC2CCCN(C(=O)OC(C)(C)C)C2)cc1CN ; CC(C)(C)OC(=O)CCC(=O)N[C@@H](Cc1c[nH]c2ccccc12)C(=O)O ; Cc1ccc(OCCC2CCCN(C(=O)OC(C)(C)C)C2)cc1CNC(=O)[C@H](Cc1c[nH]c2ccccc12)NC(=O)CCC(=O)OC(C)(C)C]

The amide bond was formed according to general procedure B with 100 mg amine 4 and 134 mg carboxylic acid 68. Purification was achieved by RP18 reversed phase flash chromatography. Formula: C₃₉H₅₄N₄O₇, exact mass: 690.4, found: 691.6 [M+H]⁺

### tert-butyl 3-(2-(3-(((S)-2-(4-(tert-butoxy)-4-oxobutanamido)propanamido)methyl)-4-methylphenoxy)ethyl)piperidine-1-carboxylate (72)

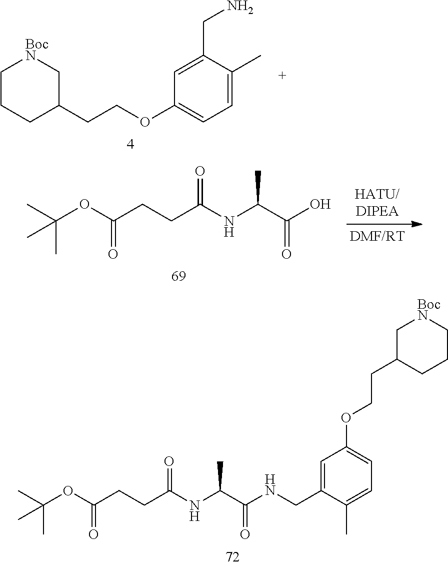 [SMILES: Cc1ccc(OCCC2CCCN(C(=O)OC(C)(C)C)C2)cc1CN ; C[C@H](NC(=O)CCC(=O)OC(C)(C)C)C(=O)O ; Cc1ccc(OCCC2CCCN(C(=O)OC(C)(C)C)C2)cc1CNC(=O)[C@H](C)NC(=O)CCC(=O)OC(C)(C)C]

The amide bond was formed according to general procedure B with 100 mg amine 4 and 91 mg carboxylic acid 69. Purification was achieved by RP18 reversed phase flash chromatography. Formula: C₃₁H₄₉N₃O₇, exact mass: 575.4, found: 576.4 [M+H]⁺

### tert-butyl 3-(2-(3-(((S)-2-(4-(tert-butoxy)-4-oxobutanamido)-3-phenylpropanamido) methyl)-4-methylphenoxy)ethyl)piperidine-1-carboxylate (73)

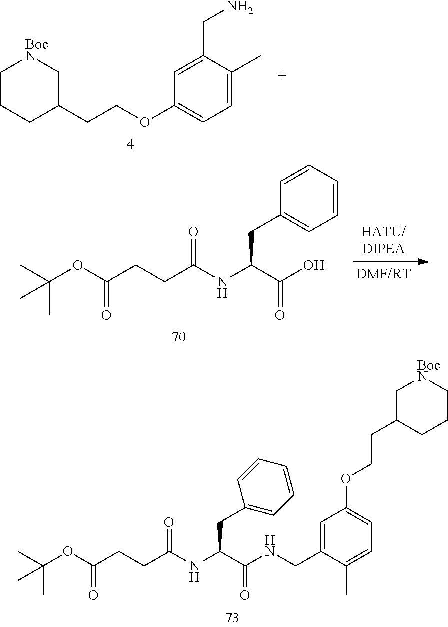 [SMILES: Cc1ccc(OCCC2CCCN(C(=O)OC(C)(C)C)C2)cc1CN ; CC(C)(C)OC(=O)CCC(=O)N[C@@H](Cc1ccccc1)C(=O)O ; Cc1ccc(OCCC2CCCN(C(=O)OC(C)(C)C)C2)cc1CNC(=O)[C@H](Cc1ccccc1)NC(=O)CCC(=O)OC(C)(C)C]

The amide bond was formed according to general procedure B with 100 mg amine 4 and 120 mg carboxylic acid 70. Purification was achieved by RP18 reversed phase flash chromatography. Formula: C₃₇H₅₃N₃O₇, exact mass: 651.4, found: 652.4 [M+H]⁺

### 4-(((2S)-3-(1H-indol-3-yl)-1-((2-methyl-5-(2-(piperidin-3-yl)ethoxy)benzyl)amino)-1-oxopropan-2-yl)amino)-4-oxobutanoic acid (74)

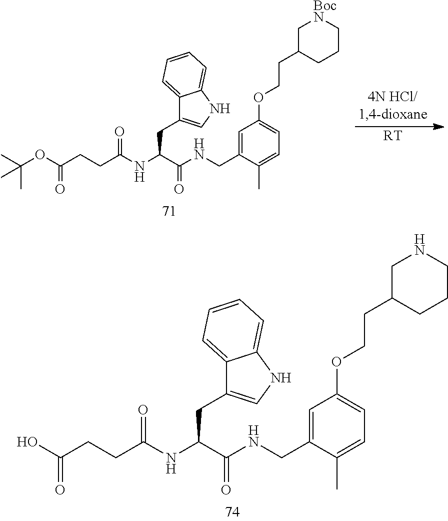 [SMILES: Cc1ccc(OCCC2CCCN(C(=O)OC(C)(C)C)C2)cc1CNC(=O)[C@H](Cc1c[nH]c2ccccc12)NC(=O)CCC(=O)OC(C)(C)C ; "RT" ; Cc1ccc(OCCC2CCCNC2)cc1CNC(=O)[C@H](Cc1c[nH]c2ccccc12)NC(=O)CCC(=O)O]

Protecting groups were cleaved according to general procedure D stirring compounds in 4 N HCl/1,4-dioxane at room temperature. After deprotection was complete solvent was removed and the crude dissolved in acetonitrile and concentrated twice. Product used without further purification.

Formula: C₃₀H₃₈N₄O₅, exact mass: 534.3, found: 536.3 [M+H]⁺

### 4-(((2S)-1-((2-methyl-5-(2-(piperidin-3-yl)ethoxy)benzyl)amino)-1-oxopropan-2-yl)amino-4-oxobutanoic acid-(75)

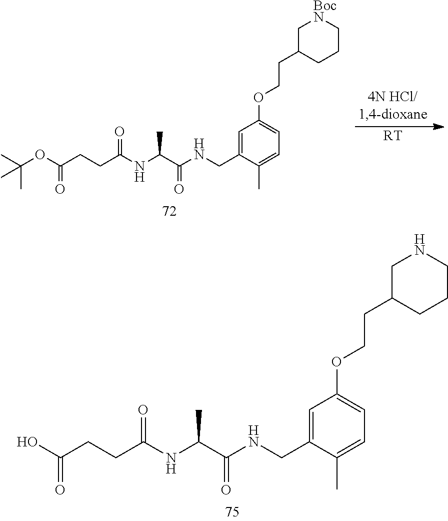 [SMILES: Cc1ccc(OCCC2CCCN(C(=O)OC(C)(C)C)C2)cc1CNC(=O)[C@H](C)NC(=O)CCC(=O)OC(C)(C)C ; "RT" ; Cc1ccc(OCCC2CCCNC2)cc1CNC(=O)[C@H](C)NC(=O)CCC(=O)O]

Protecting groups were cleaved according to general procedure D stirring compounds in 4 N HCl/1,4-dioxane at room temperature. After deprotection was complete solvent was removed and the crude dissolved in acetonitrile and concentrated twice. Product used without further purification.

Formula: C₂₂H₃₃N₃O₅, exact mass: 419.2, found: 420.1 [M+H]⁺

### 4-(((2S)-1-((2-methyl-5-(2-(piperidin-3-yl)ethoxy)benzyl)amino)-1-oxo-3-phenylpropan-2-yl)amino)-4-oxobutanoic acid (76)

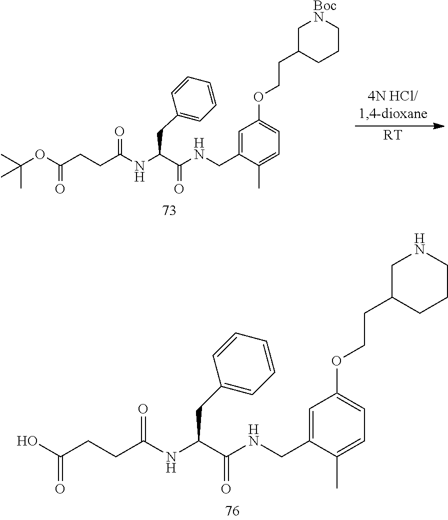 [SMILES: Cc1ccc(OCCC2CCCN(C(=O)OC(C)(C)C)C2)cc1CNC(=O)[C@H](Cc1ccccc1)NC(=O)CCC(=O)OC(C)(C)C ; "RT" ; Cc1ccc(OCCC2CCCNC2)cc1CNC(=O)[C@H](Cc1ccccc1)NC(=O)CCC(=O)O]

Protecting groups were cleaved according to general procedure D stirring compounds in 4 N HCl/1,4-dioxane at room temperature. After deprotection was complete solvent was removed and the crude dissolved in acetonitrile and concentrated twice. Product used without further purification.

Formula: C₂₈H₃₇N₃O₅, exact mass: 495.3, found: 496.3 [M+H]⁺

### (9S)-9-((1H-indol-3-yl)methyl)-5⁴-methyl-4-oxa-7,10-diaza-1(3,1)-piperidina-5(1,3)-benzenacyclotetradecaphane-8,11,14-trione (77)

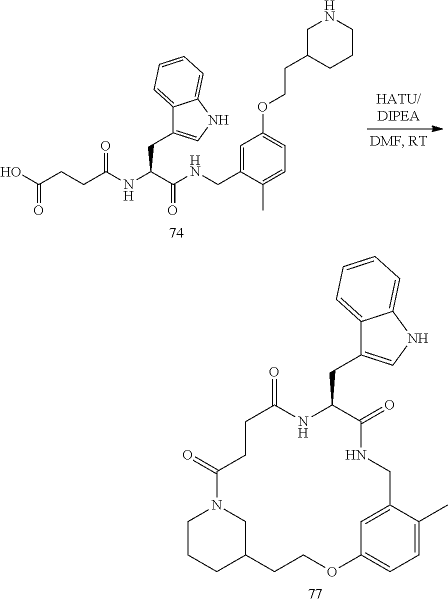 [SMILES: Cc1ccc(OCCC2CCCNC2)cc1CNC(=O)[C@H](Cc1c[nH]c2ccccc12)NC(=O)CCC(=O)O ; Cc1ccc2cc1CNC(=O)[C@H](Cc1c[nH]c3ccccc13)NC(=O)CCC(=O)N1CCCC(CCO2)C1]

Macrocyclization was achieved according to general procedure A with 0.287 mmol amino acid 74 Reaction mixtures were diluted with some methanol and purified via HPLC. Formula: C₃₀H₃₆N₄O₄, exact mass: 516.3, found: 517.3 [M+H]⁺

### (9S)-5⁴,9-dimethyl-4-oxa-7,10-diaza-1(3,1)-piperidina-5(1,3)-benzenacyclotetradecaphane-8,11,14-trione (78)

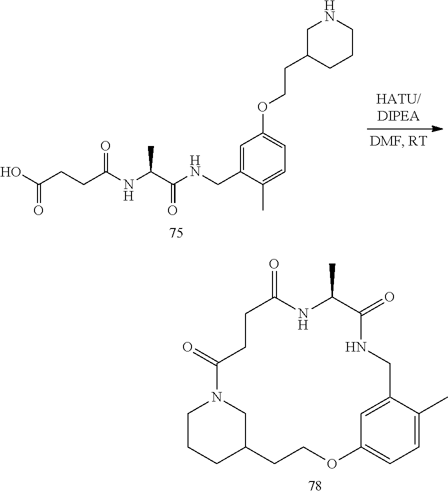 [SMILES: Cc1ccc(OCCC2CCCNC2)cc1CNC(=O)[C@H](C)NC(=O)CCC(=O)O ; Cc1ccc2cc1CNC(=O)[C@H](C)NC(=O)CCC(=O)N1CCCC(CCO2)C1]

Macrocyclization was achieved according to general procedure A with 0.224 mmol amino acid 75 Reaction mixtures were diluted with some methanol and purified via HPLC. Formula: C₂₂H₃₁N₃O₄, exact mass: 401.2, found: 402.3 [M+H]⁺

### (9S)-9-benzyl-5⁴-methyl-4-oxa-7,10-diaza-1(3,1)-piperidina-5(1,3)-benzenacyclotetradecaphane-8,11,14-trione (79)

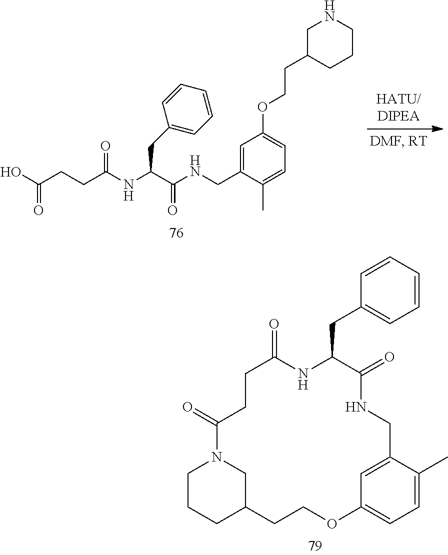 [SMILES: Cc1ccc(OCCC2CCCNC2)cc1CNC(=O)[C@H](Cc1ccccc1)NC(=O)CCC(=O)O ; Cc1ccc2cc1CNC(=O)[C@H](Cc1ccccc1)NC(=O)CCC(=O)N1CCCC(CCO2)C1]

Macrocyclization was achieved according to general procedure A with 0.215 mmol amino acid 76 Reaction mixtures were diluted with some methanol and purified via HPLC. Formula: C₂₈H₃₅N₃O₄, exact mass: 477.3, found: 478.3 [M+H]⁺

### Example A-8: Preparation of Macrocyclic Compound 95

### tert-butyl 3-(2-(3-(((S)-2-(((benzyloxy)carbonyl)amino)-4-phenylbutanamido)methyl)-4,5-dimethylphenoxy)ethyl)piperidine-1-carboxylate (91)

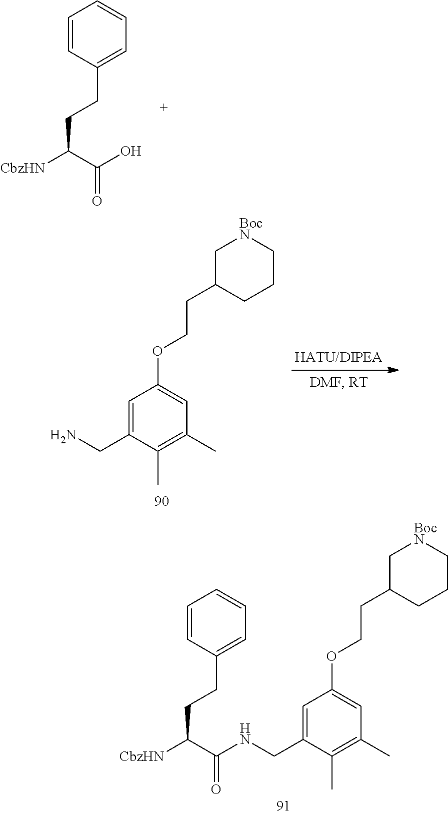 [SMILES: O=C(N[C@@H](CCc1ccccc1)C(=O)O)OCc1ccccc1 ; Cc1cc(OCCC2CCCN(C(=O)OC(C)(C)C)C2)cc(CN)c1C ; Cc1cc(OCCC2CCCN(C(=O)OC(C)(C)C)C2)cc(CNC(=O)[C@H](CCc2ccccc2)NC(=O)OCc2ccccc2)c1C]

The amide bond was formed according to general procedure B. Purification was achieved normal phase column chromatography (silica, cyclohexane/ethyl acetate gradient). Formula: C₃₉H₅₁N₃O₆, exact mass: 657.4, found: 658.6 [M+H]⁺

### tert-butyl 3-(2-(3-(((S)-2-amino-4-phenylbutanamido)methyl)-4,5-dimethylphenoxy)ethyl)-piperidine-1-carboxylate (92)

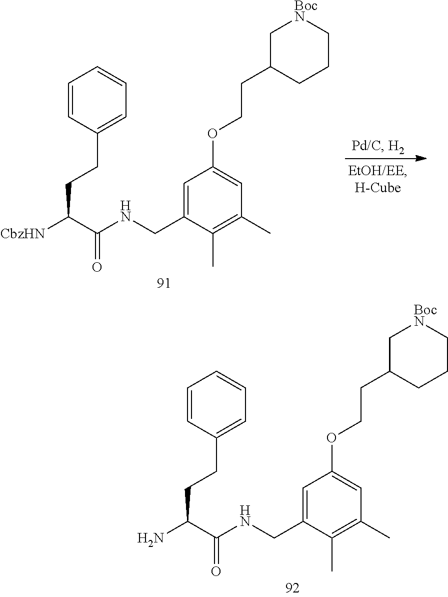 [SMILES: Cc1cc(OCCC2CCCN(C(=O)OC(C)(C)C)C2)cc(CNC(=O)[C@H](CCc2ccccc2)NC(=O)OCc2ccccc2)c1C ; Cc1cc(OCCC2CCCN(C(=O)OC(C)(C)C)C2)cc(CNC(=O)[C@@H](N)CCc2ccccc2)c1C]

Benzyl carbamate was cleaved according to general procedure C using 10% Pd/C catalyst cartridge, 20 bar at 50° C. in a solvent mixture of ethanol/ethyl acetate (1:1). Dried product was used without further purification.

Formula: C₃₁H₄₅N₃O₄, exact mass: 523.3, found: 524.6 [M+H]⁺

### tert-butyl 3-(2-(3-(((S)-2-(4-(tert-butoxy)-4-oxobutanamido)-4-phenylbutanamido)methyl)-4,5-dimethylphenoxy)ethyl)piperidine-1-carboxylate (93)

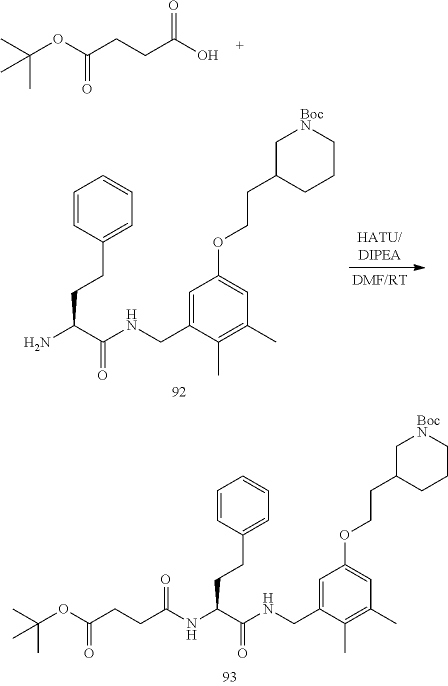 [SMILES: CC(C)(C)OC(=O)CCC(=O)O ; Cc1cc(OCCC2CCCN(C(=O)OC(C)(C)C)C2)cc(CNC(=O)[C@@H](N)CCc2ccccc2)c1C ; Cc1cc(OCCC2CCCN(C(=O)OC(C)(C)C)C2)cc(CNC(=O)[C@H](CCc2ccccc2)NC(=O)CCC(=O)OC(C)(C)C)c1C]

The amide bond was formed according to general procedure B with about 0.181 mmol amine 92 and 47 mg carboxylic acid. Purification was achieved by normal phase column chromatography (silica, cyclohexane/ethyl acetate gradient).

Formula: C₃₉H₅₇N₃O₇, exact mass: 679.4, found: 680.7 [M+H]⁺

### 4-(((2S)-1-((2,3-dimethyl-5-(2-(piperidin-3-yl)ethoxy)benzyl)amino)-1-oxo-4-phenylbutan-2-yl)amino)-4-oxobutanoic acid (94)

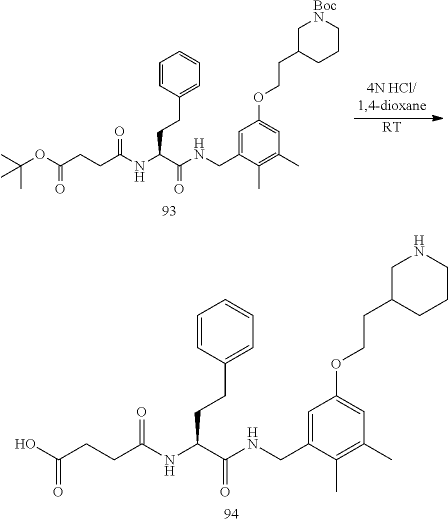 [SMILES: Cc1cc(OCCC2CCCN(C(=O)OC(C)(C)C)C2)cc(CNC(=O)[C@H](CCc2ccccc2)NC(=O)CCC(=O)OC(C)(C)C)c1C ; "RT" ; Cc1cc(OCCC2CCCNC2)cc(CNC(=O)[C@H](CCc2ccccc2)NC(=O)CCC(=O)O)c1C]

Protecting groups were cleaved according to general procedure D stirring compounds in 4 N HCl/1,4-dioxane at room temperature. After deprotection was complete solvent was removed and the crude dissolved in acetonitrile and concentrated twice. Product used without further purification.

Formula: C₃₀H₄₁N₃O₅, exact mass: 523.3, found: 524.5 [M+H]⁺

### (9S)-5⁴,5⁵-dimethyl-9-phenethyl-4-oxa-7,10-diaza-1(3,1)-piperidina-5(1,3)-benzenacyclotetradecaphane-8,11,14-trione (95)

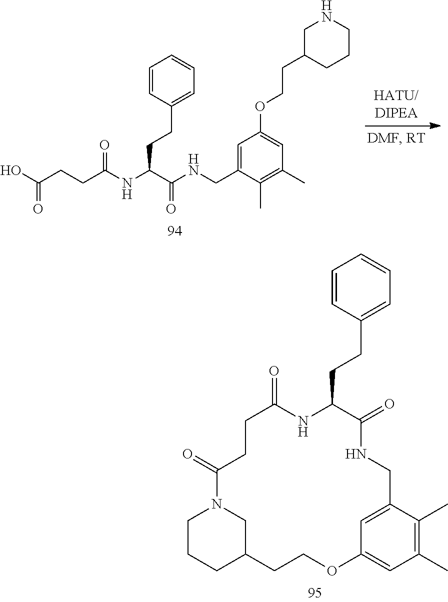 [SMILES: Cc1cc(OCCC2CCCNC2)cc(CNC(=O)[C@H](CCc2ccccc2)NC(=O)CCC(=O)O)c1C ; Cc1cc2cc(c1C)CNC(=O)[C@H](CCc1ccccc1)NC(=O)CCC(=O)N1CCCC(CCO2)C1]

Macrocyclization was achieved according to general procedure A with about 0.125 mmol amino acid 94. Reaction mixture was diluted with some methanol and purified via HPLC. Formula: C₃₀H₃₉N₃O₄, exact mass: 505.3, found: 506.4 [M+H]⁺

### Example A-9: Preparation of Macrocyclic Compounds 105, 106, 107, 371, and 372

### 2-vinylpyrazine (96)

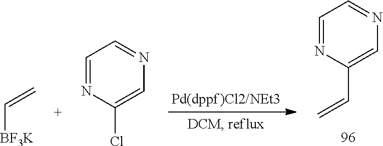

To a solution of 2-chloropyrazine (78 g, 681 mmol) in THF (800 mL) under argon atm. was added potassium vinyltrifluoroborate (137 g, 1022 mmol), triethylamine (247 mL, 2020 mmol), and Pd(dppf)Cl₂ in dichloromethane (5.6 g, 6.9 mmol). The reaction mass was refluxed for 24 h, cooled to r.t., diluted with MTBE (800 mL), and filtered through a pad of Na₂SO₄. The filtrate was evaporated under reduced pressure to obtain crude 2-vinylpyrazine (60 g, 565.5 mmol) which was used in the next step without further purification.

### diethyl 2-acetamido-2-(pyrazin-2-ylmethyl)malonate (97)

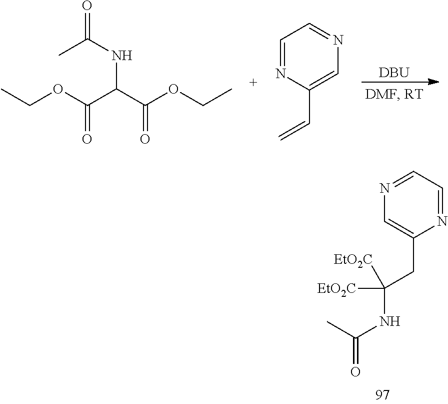

Diethyl acetamidomalonate (184.6 g, 850 mmol) and DBU (127 mL, 850 mmol) were dissolved in DMF (800 mL) and it was stirred 15 min at r.t. After that 2-vinylpyrazine (60 g, 565.5 mmol) was slowly added dropwise into reaction mixture and stirred for 24 h at r.t. Then it was concentrated under reduced pressure diluted with water (800 mL), and extracted with ethyl acetate (2×800 mL). Combined organic layers were washed with brine (3×800 mL), dried over Na₂SO₄, and concentrated to obtained 120 g of diethyl 2-acetamido-2-(pyrazin-2-ylmethyl)malonate (371 mmol, 65.6% yield) which was used in the next step without further purification.

### ethyl 2-amino-4-(pyrazin-2-yl)butanoate 98

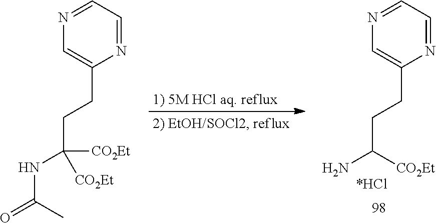

The diethyl 2-acetamido-2-(pyrazin-2-ylmethyl)malonate (120 g, 371 mmol) was dissolved in 5M hydrochloric acid (1000 mL) and refluxed for 14 h. Then the solvent was evaporated under reduced pressure to give 96 g of crude intermediate (441 mmol, 19% yield) which was used in the next step without further purification.

To cooled (5-10° C.) solution of crude from step before (441 mmol) in absolute ethanol (500 mL) was slowly added dropwise SOCl₂ (14.5 mL, 199 mmol) and stirred for 30 min. Then it was refluxed for 12 h without air access. Then the reaction mixture was concentrated under reduced pressure, and the obtained ethyl 2-amino-4-(pyrazin-2-yl)butanoate (100 g, 407 mmol) was used in the next step without further purification.

Formula: C₁₀H₁₅N₃O₂, exact mass: 209.1, found: 210.2 [M+H]⁺

### ethyl 2-(((benzyloxy)carbonyl)amino)-4-(pyrazin-2-yl)butanoate (99)

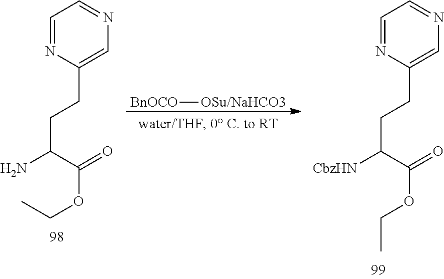

5.0 g amino ester 98 were dissolved in 25 ml water and 68 ml THF and cooled to 0° C. 5.07 g N-(benzyloxycarbonyloxy)succinimide and 8.24 g trimethylamine were added and the mixture was allow to warm to room temperature. After complete consumption of starting material the mixture was distributed between saturated NaHCO₃ solution and ethyl acetate. The organic phase was dried over MgSO₄ and volatiles were removed under reduced pressure. The crude was used without further purification.

### 2-(((benzyloxy)carbonyl)amino)-4-(pyrazin-2-yl)butanoic acid (100)

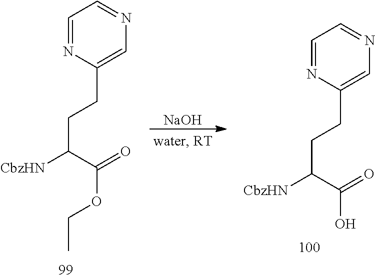 [SMILES: CCOC(=O)C(CCc1cnccn1)NC(=O)OCc1ccccc1 ; "NaOH" ; O=C(NC(CCc1cnccn1)C(=O)O)OCc1ccccc1]

6.45 g ester 99 were dissolved in 47 ml 1,4-dioxane and 20 mL 1 M NaOH solution in water at room temperature. After 3 h the mixture was brought to pH 3 with 1 M HCl solution in water. The mixture was extracted with ethyl acetate twice and combined organic phases were dried over MgSO₄. Volatiles were removed under reduced pressure. The crude was pure enough to be used in the following reactions.

Formula: C₁₆H₁₇N₃O₄, exact mass: 315.1, found: 316.1 [M+H]⁺

### tert-butyl 3-(2-(3-((2-(((benzyloxy)carbonyl)amino)-4-(pyrazin-2-yl)butanamido)methyl)-4-methylphenoxy)ethyl)piperidine-1-carboxylate (101)

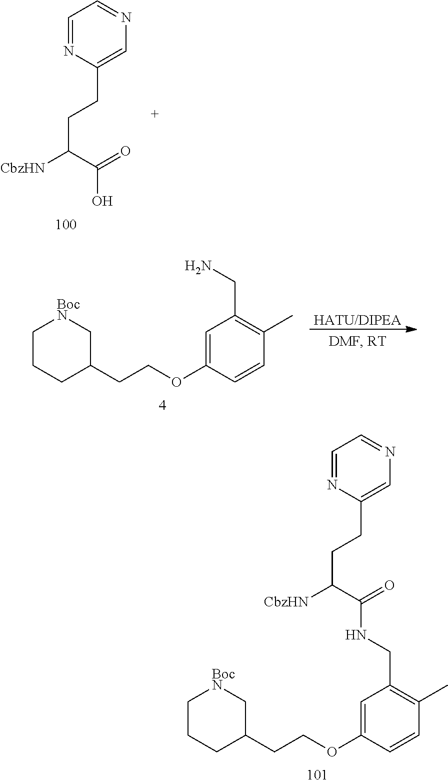 [SMILES: O=C(NC(CCc1cnccn1)C(=O)O)OCc1ccccc1 ; Cc1ccc(OCCC2CCCN(C(=O)OC(C)(C)C)C2)cc1CN ; "HATU/DIPEA" ; Cc1ccc(OCCC2CCCN(C(=O)OC(C)(C)C)C2)cc1CNC(=O)C(CCc1cnccn1)NC(=O)OCc1ccccc1]

600 mg carboxylic acid 100 was coupled with 663 mg amine 4 according to general procedure B. Purification was achieved by reversed phase column chromatography (RP18, water/acetonitrile gradient).

### tert-butyl 3-(2-(3-((2-amino-4-(pyrazin-2-yl)butanamido)methyl)-4-methylphenoxy)ethyl) piperidine-1-carboxylate (102)

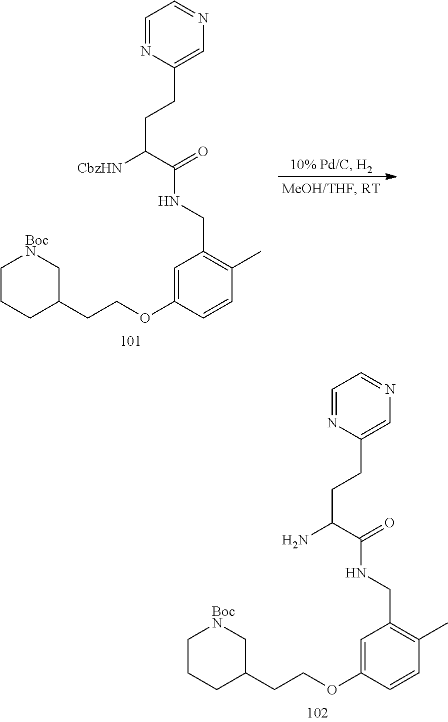 [SMILES: Cc1ccc(OCCC2CCCN(C(=O)OC(C)(C)C)C2)cc1CNC(=O)C(CCc1cnccn1)NC(=O)OCc1ccccc1 ; "10% Pd/C, H2" ; Cc1ccc(OCCC2CCCN(C(=O)OC(C)(C)C)C2)cc1CNC(=O)C(N)CCc1cnccn1]

660 mg compound 101 and 70 mg 10% Pd/C in 8 ml methanol and 1.5 ml THF were hydrogenated at room temperature under normal pressure. After complete reaction the mixture was filtrated and volatiles were removed. Purification was achieved by normal phase column chromatography (silica, cyclohexane/ethyl acetate gradient).

Formula: C₂₈H₄₁N₅O₄, exact mass: 511.3, found: 512.3 [M+H]⁺

### tert-butyl 3-(2-(3-((5S)-5-(2-(tert-butoxy)-2-oxoethyl)-3,6,9-trioxo-1-phenyl-8-(2-(pyrazin-2-yl)ethyl)-2-oxa-4,7,10-triazaundecan-11-yl)-4-methylphenoxy)ethyl)piperidine-1-carboxylate (103)

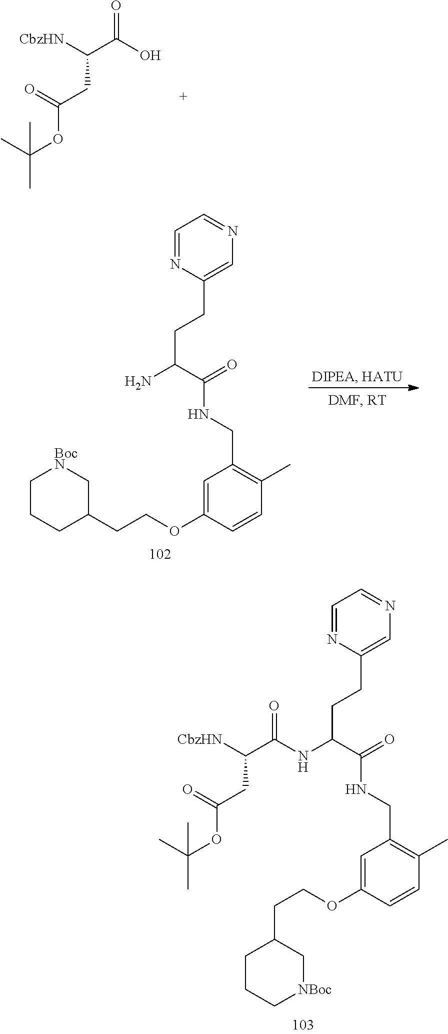 [SMILES: CC(C)(C)OC(=O)C[C@H](NC(=O)OCc1ccccc1)C(=O)O ; Cc1ccc(OCCC2CCCN(C(=O)OC(C)(C)C)C2)cc1CNC(=O)C(N)CCc1cnccn1 ; "DIPEA, HATU" ; Cc1ccc(OCCC2CCCN(C(=O)OC(C)(C)C)C2)cc1CNC(=O)C(CCc1cnccn1)NC(=O)[C@H](CC(=O)OC(C)(C)C)NC(=O)OCc1ccccc1]

The amide bond was formed according to general procedure B with 243 mg amine 102 and 230 mg Cbz-Asp(OtBu)-OH. Purification was achieved by normal phase column chromatography (silica, cyclohexane/ethyl acetate gradient).

Formula: C₄₄H₆₀N₆O₉, exact mass: 816.4, found: 717.6 [M+H-Boc]⁺

### (3S)-3-(((benzyloxy)carbonyl)amino)-4-((1-((2-methyl-5-(2-(piperidin-3-yl)ethoxy)benzyl)amino)-1-oxo-4-(pyrazin-2-yl)butan-2-yl)amino)-4-oxobutanoic acid (104)

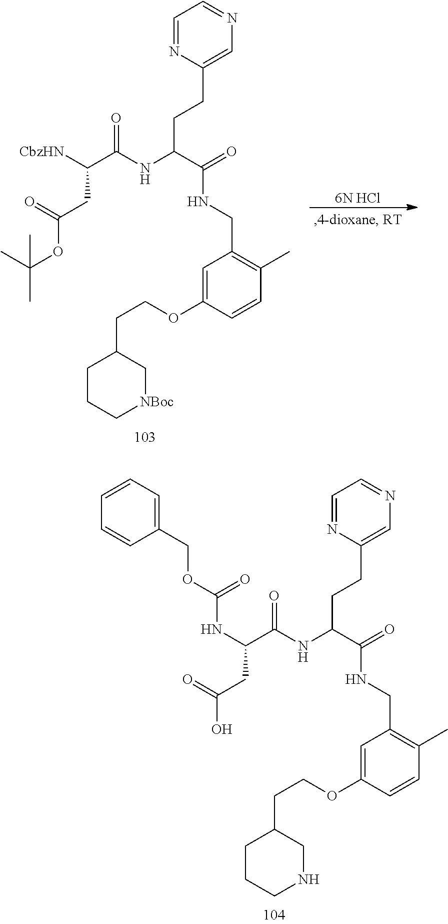 [SMILES: Cc1ccc(OCCC2CCCN(C(=O)OC(C)(C)C)C2)cc1CNC(=O)C(CCc1cnccn1)NC(=O)[C@H](CC(=O)OC(C)(C)C)NC(=O)OCc1ccccc1 ; "6N HCl" ; Cc1ccc(OCCC2CCCNC2)cc1CNC(=O)C(CCc1cnccn1)NC(=O)[C@H](CC(=O)O)NC(=O)OCc1ccccc1]

Protecting groups were cleaved from 280 mg compound 103 according to general procedure D stirring compounds in 10 ml 6 N HCl/1,4-dioxane at room temperature. After deprotection was complete solvent was removed and the crude dissolved in acetonitrile and concentrated twice. Product used without further purification.

Formula: C₃₅H₄₄N₆O₇, exact mass: 660.3, found: 662.3 [M+H]⁺

### benzyl ((12S)-5⁴-methyl-8,11,14-trioxo-9-(2-(pyrazin-2-yl)ethyl)-4-oxa-7,10-diaza-1(3,1)-piperidina-5(1,3)-benzenacyclotetradecaphane-12-yl)carbamate (105)

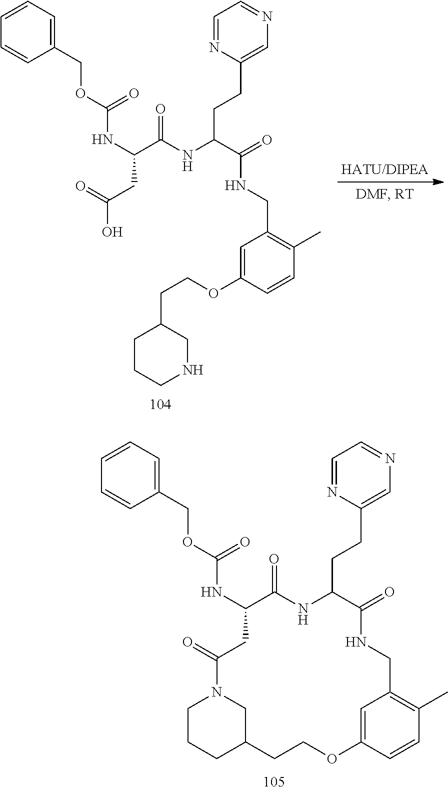 [SMILES: Cc1ccc(OCCC2CCCNC2)cc1CNC(=O)C(CCc1cnccn1)NC(=O)[C@H](CC(=O)O)NC(=O)OCc1ccccc1 ; Cc1ccc2cc1CNC(=O)C(CCc1cnccn1)NC(=O)[C@@H](NC(=O)OCc1ccccc1)CC(=O)N1CCCC(CCO2)C1]

Macrocyclization was achieved according to general procedure A with 0.343 mmol amino acid 58. Reaction mixtures were diluted with some methanol and purified by reversed phase column chromatography (RP18, water/methanol gradient).

Formula: C₃₅H₄₂N₆O₆, exact mass: 642.3, found: 643.5 [M+H]⁺

### (12S)-amino-5⁴-methyl-8,11,14-trioxo-9-(2-(pyrazin-2-yl)ethyl)-4-oxa-7,10-diaza-1(3,1)-piperidina-5(13)-benzenacyclotetradecaphane (106)

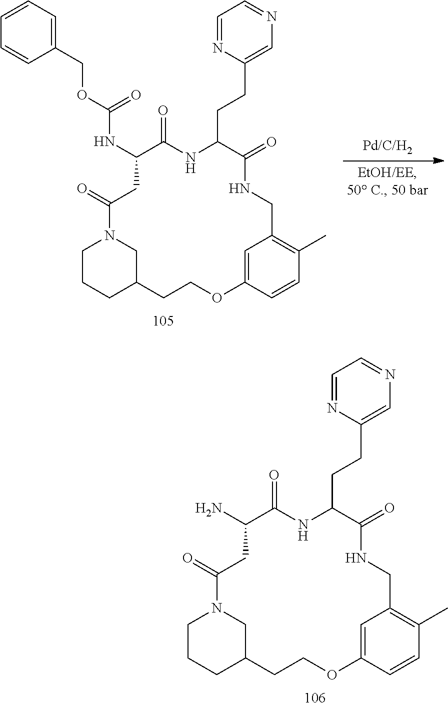 [SMILES: Cc1ccc2cc1CNC(=O)C(CCc1cnccn1)NC(=O)[C@@H](NC(=O)OCc1ccccc1)CC(=O)N1CCCC(CCO2)C1 ; Cc1ccc2cc1CNC(=O)C(CCc1cnccn1)NC(=O)[C@@H](N)CC(=O)N1CCCC(CCO2)C1]

Benzyl carbamate was cleaved according to general procedure C using 10% Pd/C catalyst cartridge, 20 bar at 50° C. in a solvent mixture of ethanol/ethyl acetate (3:1). After removing of volatiles the residue was directly used in the next step.

Formula: C₂₇H₃₆N₆O₄, exact mass: 508.3, found: −[M+H]⁺

### (2S)—N-((12S)-5⁴-methyl-8,11,14-trioxo-9-(2-(pyrazin-2-yl)ethyl)-4-oxa-7,10-diaza-1(3,1)-piperidina-5(1,3)-benzenacyclotetradecaphane-12-yl)-2-phenylpropanamide (107)

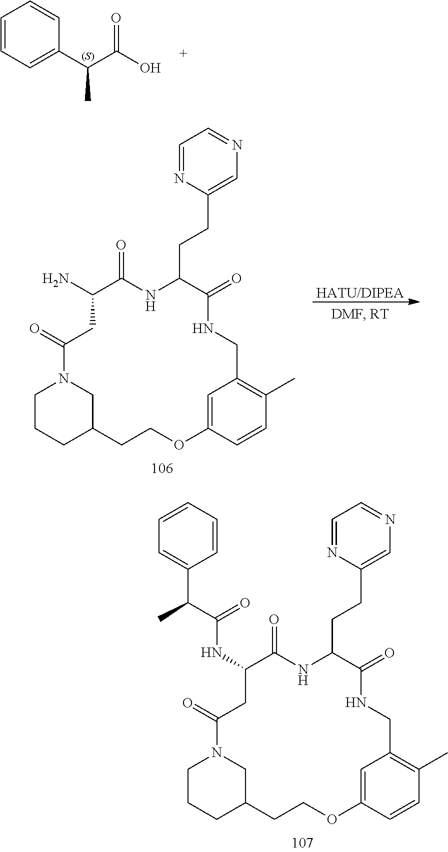

The amide bond was formed according to general procedure B with e.g. 25 mg amine 106 and 11 mg carboxylic acid. Purification was achieved by revered phase HPLC.

Formula: C₃₆H₄₄N₆O₅, exact mass: 640.3, found: 641.4 [M+H]⁺

Additional examples as exemplified by compound 107 are disclosed in the table below. In the cases of compounds 371 and 372 Boc groups were cleaved from the related Boc-protected intermediates with 40% TFA in DCM before final purification via HPLC.

TABLE A-5

| Cpd | | | Exact | [M + H]⁺ |
|---|---|---|---|---|
| no | structure | name | mass | found |
| 107 | 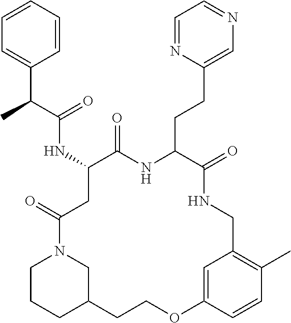 [SMILES: Cc1ccc2cc1CNC(=O)C(CCc1cnccn1)NC(=O)[C@@H](NC(=O)[C@@H](C)c1ccccc1)CC(=O)N1CCCC(CCO2)C1] | (2S)-N-((12S)-5⁴-methyl-8,11,14- trioxo-9-(2-(pyrazin-2-yl)ethyl)-4-oxa- 7,10-diaza-1(3,1)-piperidina-5(1,3)- benzenacyclotetradecaphane-12-yl)- 2-phenylpropanamide | 640.3 C₃₆H₄₄ N₆O₅ | 641.4 |
| 371 | 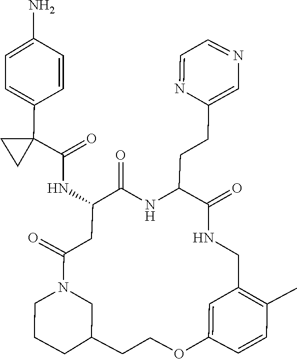 [SMILES: Cc1ccc2cc1CNC(=O)C(CCc1cnccn1)NC(=O)[C@@H](NC(=O)C1(c3ccc(N)cc3)CC1)CC(=O)N1CCCC(CCO2)C1] | 1-(4-aminophenyl)-N-((9S,12S)-5⁴- methyl-8,11,14-trioxo-9-(2-(pyrazin- 2-yl)ethyl)-4-oxa-7,10-diaza-1(3,1)- piperidina-5(1,3)- benzenacyclotetradecaphane-12- yl)cyclopropane-1-carboxamide | 667.3 C₃₇H₄₅ N₇O₅ | 668.5 |
| 372 | 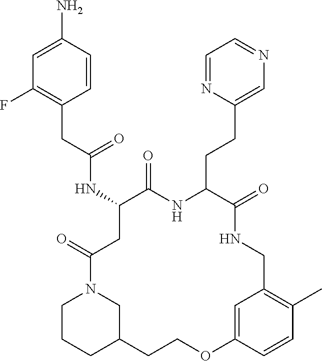 [SMILES: Cc1ccc2cc1CNC(=O)C(CCc1cnccn1)NC(=O)[C@@H](NC(=O)Cc1ccc(N)cc1F)CC(=O)N1CCCC(CCO2)C1] | 2-(4-amino-2-fluorophenyl)-N- ((9S,12S)-5⁴-methyl-8,11,14-trioxo- 9-(2-(pyrazin-2-yl)ethyl)-4-oxa-7,10- diaza-1(3,1)-piperidina-5(1,3)- benzenacyclotetradecaphane-12- yl) acetamide | 659.3 C₃₅H₄₂F N₇O₅ | 660.4 |

### Example A-10: Preparation of Compound (116)

### ethyl 4-(3-(2-(3-(aminomethyl)-4-methylphenoxy)ethyl)piperidin-1-yl)-4-oxobutanoate (112)

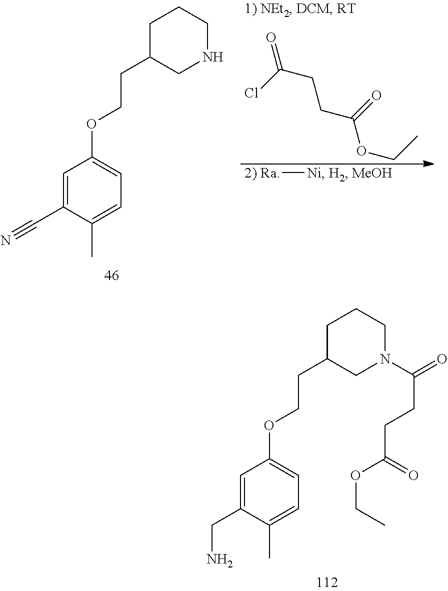

2.48 g amine 46 and 5.64 ml NEt₃ were dissolved in 60 ml DCM and cooled to 0° C. 1.86 g ethyl 4-chloro-4-oxobutanoate were added dropwise and the mixture was allowed to come to room temperature. After complete consumption of the starting amine saturated NaHCO₃ solution was added and the mixture was extracted with DCM. The organic phase was dried and concentrated under reduced pressure. Purification of the residue was achieved by normal phase column chromatography (silica, cyclohexane/ethyl acetate gradient).

2.0 g of the intermediate ester were dissolved in 135 ml methanol and hydrogenated with 7.0 Raney nickel under 50 bar at 70° C. The mixture was filtered and concentrated under reduced pressure. The residue was pure enough to be used in the following reactions (mixture of methyl and ethyl ester).

Formula: C₂₁H₃₂N₂O₄, exact mass: 376.2, found: 377.3 [M+H]⁺

### ethyl 2-amino-2-(1-methyl-1H-pyrazol-4-yl)acetate (109)

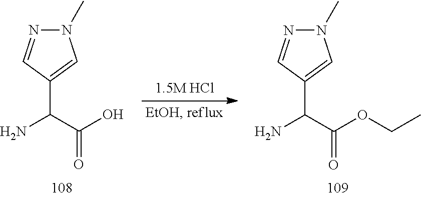

3.75 g 2-amino-2-(1-methyl-1H-pyrazol-4-yl)acetic acid are refluxed in 60 ml 1.25 M HCl in ethanol until esterification was found to be complete. Volatiles were removed under reduced pressure and the residue was coevaporated with acetonitrile twice. The crude was used without further purification.

Formula: C₈H₁₃N₃O₂, exact mass: 183.1, found: 184.3 [M+H]⁺

### ethyl 2-(((benzyloxy)carbonyl)amino)-2-(1-methyl-1H-pyrazol-4-yl)acetate (110)

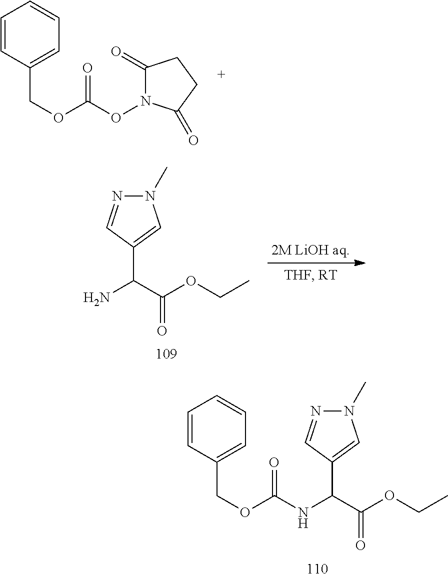

24 mmol amino ester 109 was dissolved in THF/water (3:1) at 0° C. 6.0 g CbzOSu and 13.1 ml NEt₃ were added and the mixtures was allowed to come to room temperature. The mixture was distributed between ethyl acetate and saturated NaHCO₃ solution. The organic phase was washed with 2 N HCl, dried over Na₂SO₄ and concentrated. The crude was purified by normal phase column chromatography (silica, cyclohexane/ethyl acetate gradient) to give 3.54 g ester 110.

Formula: C₁₆H₁₉N₃O₄, exact mass: 317.1, found: 318.3 [M+H]⁺

### 2-(((benzyloxy)carbonyl)amino)-2-(1-methyl-1H-pyrazol-4-yl)acetic acid (111)

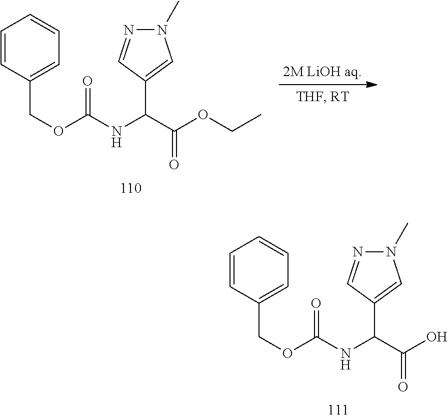 [SMILES: CCOC(=O)C(NC(=O)OCc1ccccc1)c1cnn(C)c1 ; Cn1cc(C(NC(=O)OCc2ccccc2)C(=O)O)cn1]

3.4 g ester 110 were dissolved in 12 ml THF and 4 ml 2 M LiOH aq. were added. Some additional water was added to obtain only one phase. After completion of reaction the mixture was washed with ethyl acetate and the aqueous phase was acified to pH 2. Extraction with ethyl acetate and removing the solvent under reduced pressure afforded 2.1 g Cbz protected amino acid 111.

Formula: C₁₄H₁₅N₃O₄, exact mass: 289.1, found: 290.3 [M+H]⁺

### ethyl 4-(3-(2-(3-((2-(((benzyloxy)carbonyl)amino)-2-(1-methyl-1H-pyrazol-4-yl)acetamido)methyl)-4-methylphenoxy)ethyl)piperidin-1-yl)-4-oxobutanoate (113)

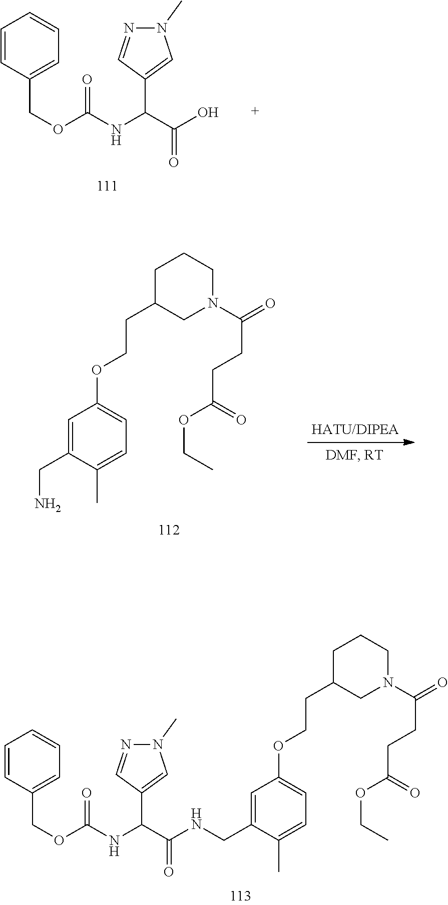 [SMILES: Cn1cc(C(NC(=O)OCc2ccccc2)C(=O)O)cn1 ; CCOC(=O)CCC(=O)N1CCCC(CCOc2ccc(C)c(CN)c2)C1 ; CCOC(=O)CCC(=O)N1CCCC(CCOc2ccc(C)c(CNC(=O)C(NC(=O)OCc3ccccc3)c3cnn(C)c3)c2)C1]

Peptide coupling was done according to general procedure B with 468 mg amine 112 and 300 mg amino acid 111. Purification was achieved by normal phase column chromatography (silica, cyclohexane/ethyl acetate gradient).

Formula: C₃₅H₄₅N₅O₇, exact mass: 647.3, found: 648.3 [M+H]⁺

### ethyl 4-(3-(2-(3-((2-amino-2-(1-methyl-1H-pyrazol-4-yl)acetamido)methyl)-4-methylphenoxy) ethyl)piperidin-1-yl)-4-oxobutanoate (114)

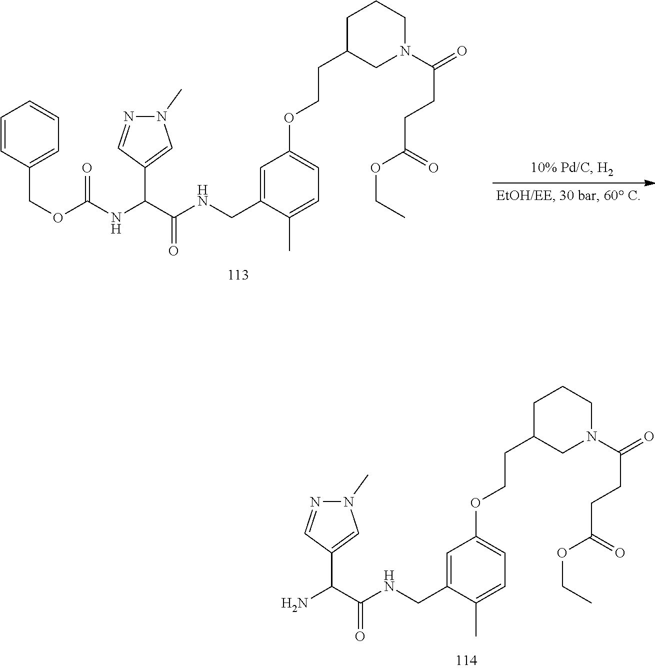 [SMILES: CCOC(=O)CCC(=O)N1CCCC(CCOc2ccc(C)c(CNC(=O)C(NC(=O)OCc3ccccc3)c3cnn(C)c3)c2)C1 ; CCOC(=O)CCC(=O)N1CCCC(CCOc2ccc(C)c(CNC(=O)C(N)c3cnn(C)c3)c2)C1]

From 647 mg compound 113 the benzyl carbamate was cleaved according to general procedure C using 10% Pd/C catalyst cartridge, 30 bar at 60° C. in a solvent mixture of ethanol/ethyl acetate (1:1). Product was used without further purification after removing the volatiles.

Formula: C₂₇H₃₉N₅O₅, exact mass: 513.3, found: 514.3 [M+H]⁺

### 4-(3-(2-(3-((2-amino-2-(1-methyl-1H-pyrazol-4-yl)acetamido)methyl)-4-methylphenoxy)ethyl)piperidin-1-yl)-4-oxobutanoic acid (115)

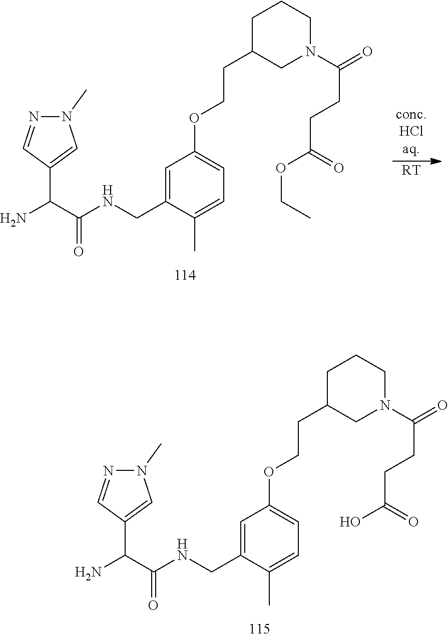 [SMILES: CCOC(=O)CCC(=O)N1CCCC(CCOc2ccc(C)c(CNC(=O)C(N)c3cnn(C)c3)c2)C1 ; "RT" ; Cc1ccc(OCCC2CCCN(C(=O)CCC(=O)O)C2)cc1CNC(=O)C(N)c1cnn(C)c1]

1.04 mmol ester 114 were dissolved in 20 ml concentrated aqueous HCl solution at room temperature. After complete reaction volatiles were removed under reduced pressure and the crude was directly used in the next step.

Formula: C₂₂H₃₅N₅O₅, exact mass: 485.3, found: 486.2 [M+H]⁺

### 5⁴-methyl-9-(1-methyl-1H-pyrazol-4-yl)-4-oxa-7,10-diaza-1(3,1)-piperidina-5(1,3)-benzenacyclotetradecaphane-8,11,14-trione (116)

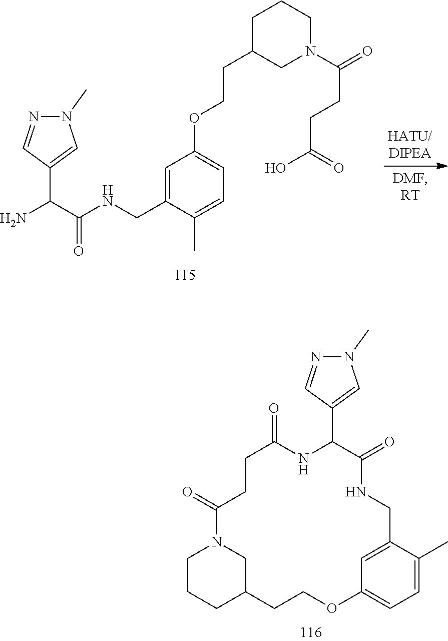 [SMILES: Cc1ccc(OCCC2CCCN(C(=O)CCC(=O)O)C2)cc1CNC(=O)C(N)c1cnn(C)c1 ; Cc1ccc2cc1CNC(=O)C(c1cnn(C)c1)NC(=O)CCC(=O)N1CCCC(CCO2)C1]

Macrocyclization was achieved according to general procedure A with 1.04 mmol amino acid 115. Reaction mixtures were diluted with some methanol and purified via HPLC.

Formula: C₂₅H₃₃N₅O₄, exact mass: 467.3, found: 468.3 [M+H]⁺

### Example A-11: Preparation of Compound (120)

### 2,3-dimethyl-5-(2-(piperidin-3-yl)ethoxy)benzonitrile (574)

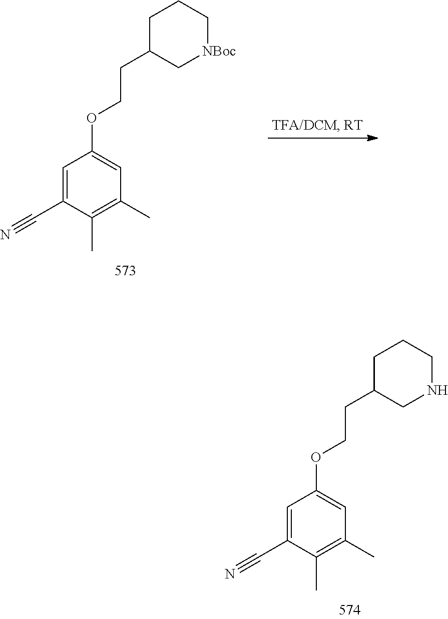 [SMILES: Cc1cc(OCCC2CCCN(C(=O)OC(C)(C)C)C2)cc(C#N)c1C ; Cc1cc(OCCC2CCCNC2)cc(C#N)c1C]

Boc group was removed from 1.77 g of compound 573 according general procedure D using 2.8 g TFA in 49 ml DCM. Volatiles were removed, the residue was dissolved in DCM and washed with 5% aqueous NaOH solution. The organic phase was dried over MgSO₄, filtered and the solvent was removed under reduced pressure. The crude was used without further purification.

### ethyl 4-(3-(2-(3-cyano-4,5-dimethylphenoxy)ethyl)piperidin-1-yl)-4-oxobutanoate (575)

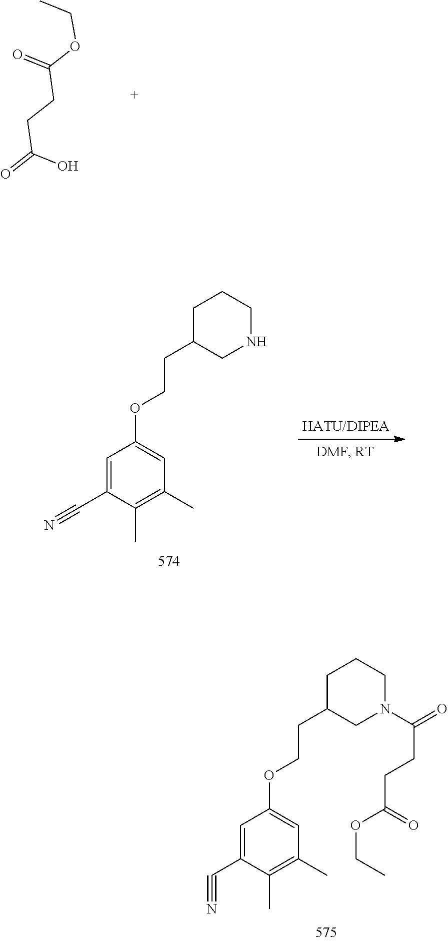

The amide bond was formed according to general procedure B with amine 574. Purification was achieved by normal phase flash chromatography.

### ethyl 4-(3-(2-(3-(aminomethyl)-4,5-dimethylphenoxy)ethyl)piperidin-1-yl)-4-oxobutanoate (117)

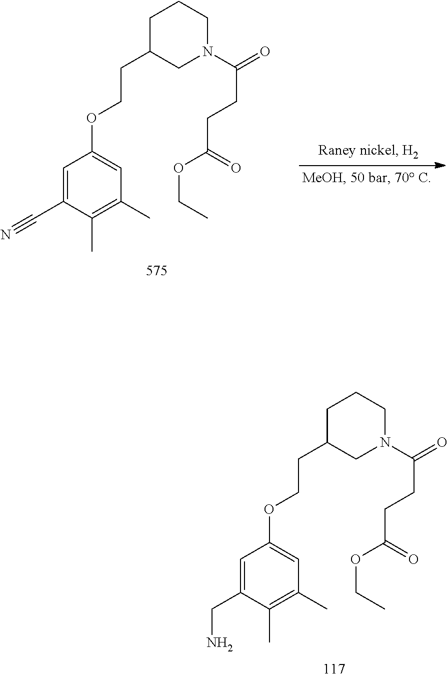 [SMILES: CCOC(=O)CCC(=O)N1CCCC(CCOc2cc(C)c(C)c(C#N)c2)C1 ; CCOC(=O)CCC(=O)N1CCCC(CCOc2cc(C)c(C)c(CN)c2)C1]

536 mg of the intermediate nitrile 575 were dissolved in 36 ml methanol and hydrogenated with 1.9 g Raney nickel at 70° C. and 50 bar. The mixture was filtered and concentrated under reduced pressure. The residue was pure enough to be used in the following reactions (mixture of methyl and ethyl ester).

Formula: C₂₂H₃₄N₂O₄, exact mass: 390.3, found: 391.3 [M+H]⁺

### tert-butyl 3-((2S)-2-((tert-butoxycarbonyl)amino)-3-((5-(2-(1-(4-ethoxy-4-oxobutanoyl)piperidin-3-yl)ethoxy)-2,3-dimethylbenzyl)amino)-3-oxopropyl)-1H-indole-1-carboxylate (118)

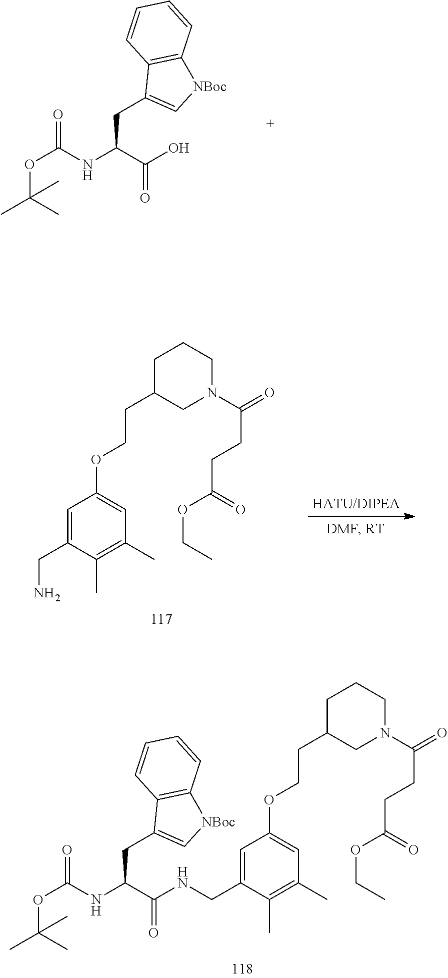 [SMILES: CC(C)(C)OC(=O)N[C@@H](Cc1cn(C(=O)OC(C)(C)C)c2ccccc12)C(=O)O ; CCOC(=O)CCC(=O)N1CCCC(CCOc2cc(C)c(C)c(CN)c2)C1 ; CCOC(=O)CCC(=O)N1CCCC(CCOc2cc(C)c(C)c(CNC(=O)[C@H](Cc3cn(C(=O)OC(C)(C)C)c4ccccc34)NC(=O)OC(C)(C)C)c2)C1]

Condensation was achieved according to general procedure B with 232 mg amine 117 and 312 mg Boc protected amino acid. Purification was achieved by normal phase column chromatography (silica, cyclohexane/ethyl acetate gradient).

Formula: C₄₃H₆₀N₄O₉, exact mass: 776.4, found: 777.5 [M+H]⁺

### 4-(3-(2-(3-(((S)-2-amino-3-(1H-indol-3-yl)propanamido)methyl)-4,5-dimethylphenoxy)ethyl)piperidin-1-yl)-4-oxobutanoic acid (119)

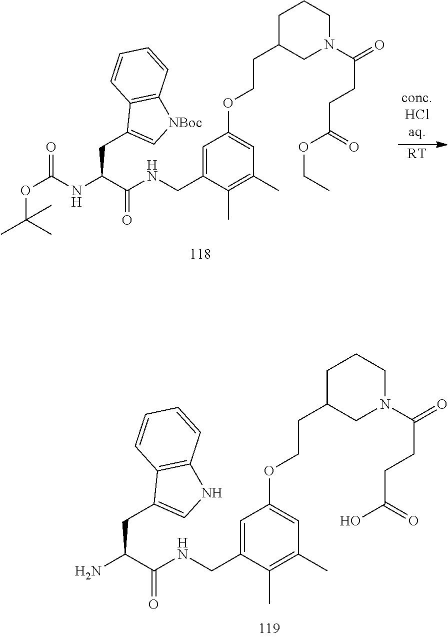 [SMILES: CCOC(=O)CCC(=O)N1CCCC(CCOc2cc(C)c(C)c(CNC(=O)[C@H](Cc3cn(C(=O)OC(C)(C)C)c4ccccc34)NC(=O)OC(C)(C)C)c2)C1 ; "RT" ; Cc1cc(OCCC2CCCN(C(=O)CCC(=O)O)C2)cc(CNC(=O)[C@@H](N)Cc2c[nH]c3ccccc23)c1C]

Protecting groups were cleaved from 240 mg compound 118 according to general procedure D stirring compounds in 10 ml concentrated aqueous HCl solution at room temperature. After deprotection was complete solvent was removed and the crude dissolved in acetonitrile and concentrated twice. Product used without further purification.

Formula: C₃₁H₄₀N₄O₅, exact mass: 548.3, found: 549.4 [M+H]⁺

### (9S)-9-((1H-indol-3-yl)methyl)-5⁴,5⁵-dimethyl-4-oxa-7,10-diaza-1(3,1)-piperidina-5(1,3)-benzenacyclotetradecaphane-8,11,14-trione (120)

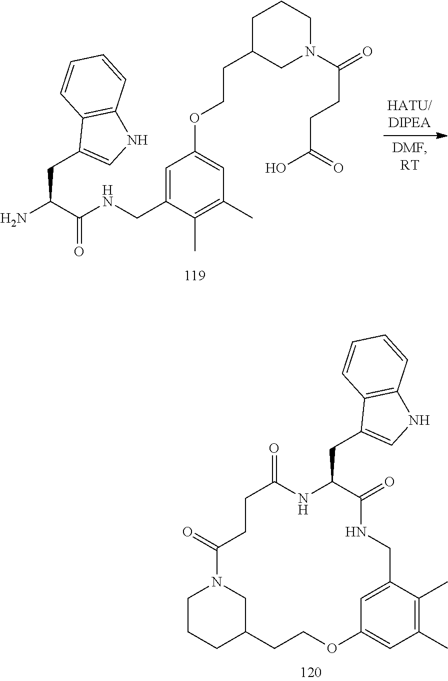 [SMILES: Cc1cc(OCCC2CCCN(C(=O)CCC(=O)O)C2)cc(CNC(=O)[C@@H](N)Cc2c[nH]c3ccccc23)c1C ; Cc1cc2cc(c1C)CNC(=O)[C@H](Cc1c[nH]c3ccccc13)NC(=O)CCC(=O)N1CCCC(CCO2)C1]

Macrocyclization was achieved according to general procedure A with 0.309 mmol amino acid 119. Reaction mixture was diluted with some methanol and purified via HPLC.

Formula: C₃₁H₃₈N₄O₄, exact mass: 530.3, found: 531.4 [M+H]⁺

### Example A-12: Derivatizations of Compound 8 with Boc-Protected Amino Acids

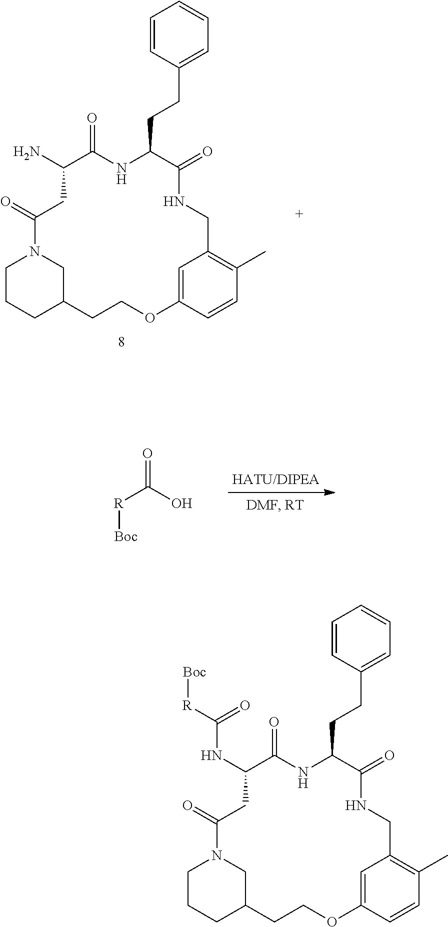

Amine 8 (e.g. 20 to 100 mg) was coupled with carboxylic acids according to general procedure B to give amides disclosed in table below. Purifications were achieved by normal phase column chromatography (silica, cyclohexane/ethyl acetate gradient).

TABLE A-6

| Cpd | | | Exact | [M + H]⁺ |
|---|---|---|---|---|
| no | structure | name | mass | found |
| 121 | 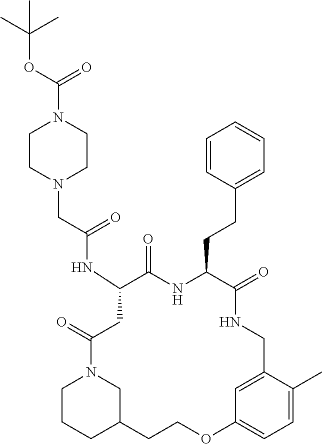 [SMILES: Cc1ccc2cc1CNC(=O)[C@H](CCc1ccccc1)NC(=O)[C@@H](NC(=O)CN1CCN(C(=O)OC(C)(C)C)CC1)CC(=O)N1CCCC(CCO2)C1] | tert-butyl 4-(2-(((9S,12S)-5⁴- methyl-8,11,14-trioxo-9- phenethyl-4-oxa-7,10-diaza- 1(3,1)-piperidina-5(1,3)- benzenacyclotetradecaphane- 12-yl)amino)-2-oxoethyl) piperazine-1-carboxylate | 732.4 C₄₀H₅₆N₆O₇ | 733.5 |
| 122 | 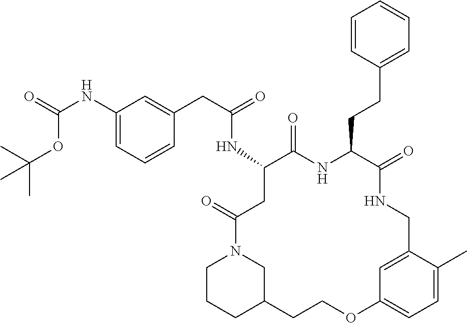 [SMILES: Cc1ccc2cc1CNC(=O)[C@H](CCc1ccccc1)NC(=O)[C@@H](NC(=O)Cc1cccc(NC(=O)OC(C)(C)C)c1)CC(=O)N1CCCC(CCO2)C1] | tert-butyl (3-(2-(((9S,12S)-5⁴- methyl-8,11,14-trioxo-9- phenethyl-4-oxa-7,10-diaza- 1(3,1)-piperidina-5(1,3)- benzenacyclotetradecaphane- 12-yl)amino)-2- oxoethyl)phenyl)carbamate | 739.4 C₄₂H₅₃N₅O₇ | 740.6 |
| 123 | 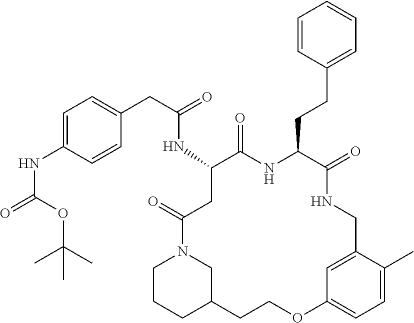 [SMILES: Cc1ccc2cc1CNC(=O)[C@H](CCc1ccccc1)NC(=O)[C@@H](NC(=O)Cc1ccc(NC(=O)OC(C)(C)C)cc1)CC(=O)N1CCCC(CCO2)C1] | tert-butyl (4-(2-(((9S,12S)-5⁴- methyl-8,11,14-trioxo-9- phenethyl-4-oxa-7,10-diaza- 1(3,1)-piperidina-5(1,3)- benzenacyclotetradecaphane- 12-yl)amino)-2- oxoethyl)phenyl)carbamate | 739.4 C₄₂H₅₃N₅O₇ | 740.7 |
| 124 | 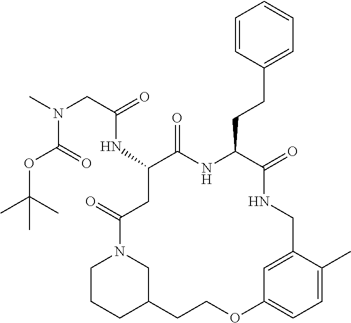 [SMILES: Cc1ccc2cc1CNC(=O)[C@H](CCc1ccccc1)NC(=O)[C@@H](NC(=O)CN(C)C(=O)OC(C)(C)C)CC(=O)N1CCCC(CCO2)C1] | tert-butyl methyl(2- (((9S,12S)-5⁴-methyl- 8,11,14-trioxo-9- phenethyl-4-oxa-7,10-diaza- 1(3,1)-piperidina-5(1,3)- benzenacyclotetradecaphane- 12-yl)amino)-2-oxoethyl) carbamate | 677.4 C₃₇H₅₁N₅O₇ | 678.5 |
| 125 | 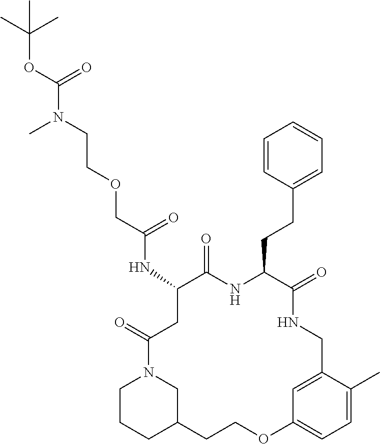 [SMILES: Cc1ccc2cc1CNC(=O)[C@H](CCc1ccccc1)NC(=O)[C@@H](NC(=O)COCCN(C)C(=O)OC(C)(C)C)CC(=O)N1CCCC(CCO2)C1] | tert-butyl methyl(2-(2- (((9S,12S)-5⁴-methyl- 8,11,14-trioxo-9- phenethyl-4-oxa-7,10-diaza- 1(3,1)-piperidina-5(1,3)- benzenacyclotetradecaphane- 12-yl)amino)-2- oxoethoxy)ethyl)carbamate | 721.4 C₃₉H₅₅N₅O₈ | 722.6 |
| 131 | 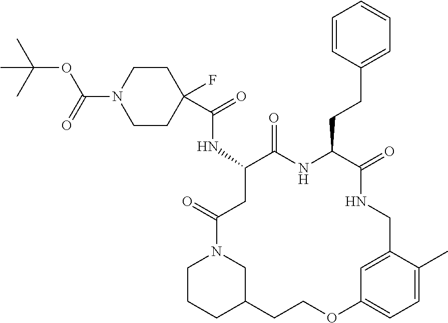 [SMILES: Cc1ccc2cc1CNC(=O)[C@H](CCc1ccccc1)NC(=O)[C@@H](NC(=O)C1(F)CCN(C(=O)OC(C)(C)C)CC1)CC(=O)N1CCCC(CCO2)C1] | tert-butyl 4-fluoro-4- (((9S,12S)-5⁴-methyl- 8,11,14-trioxo-9- phenethyl-4-oxa-7,10-diaza- 1(3,1)-piperidina-5(1,3)- benzenacyclotetradecaphane- 12-yl)carbamoyl)piperidine-1- carboxylate | 735.4 C₄₀H₅₄FN₅O₇ | 736.7 |
| 132 | 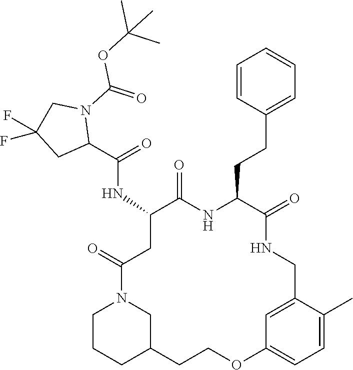 [SMILES: Cc1ccc2cc1CNC(=O)[C@H](CCc1ccccc1)NC(=O)[C@@H](NC(=O)C1CC(F)(F)CN1C(=O)OC(C)(C)C)CC(=O)N1CCCC(CCO2)C1] | tert-butyl 4,4-difluoro-2- (((9S,12S)-5⁴-methyl-8,11,14- trioxo-9-phenethyl-4-oxa-7,10- diaza-1(3,1)-piperidina-5(1,3)- benzenacyclotetradecaphane- 12-yl)carbamoyl)pyrrolidine-1- carboxylate | 739.4 C₃₉H₅₁F₂N₅O₇ | 740.6 |
| 133 | 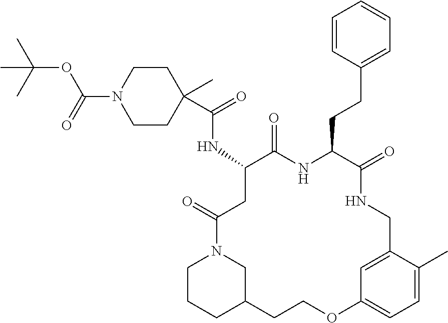 [SMILES: Cc1ccc2cc1CNC(=O)[C@H](CCc1ccccc1)NC(=O)[C@@H](NC(=O)C1(C)CCN(C(=O)OC(C)(C)C)CC1)CC(=O)N1CCCC(CCO2)C1] | tert-butyl 4-methyl-4- (((9S,12S)-5⁴-methyl- 8,11,14-trioxo-9- phenethyl-4-oxa-7,10-diaza- 1(3,1)-piperidina-5(1,3)- benzenacyclotetradecaphane- 12-yl)carbamoyl)piperidine-1- carboxylate | 731.4 C₄₁H₅₇N₅O₇ | 732.8 |
| 149 | 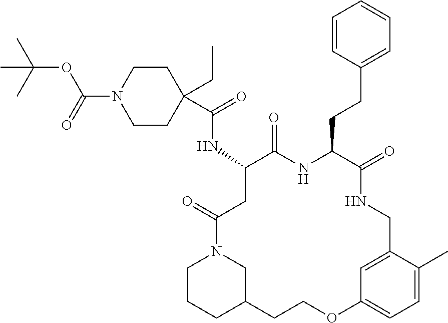 [SMILES: CCC1(C(=O)N[C@H]2CC(=O)N3CCCC(CCOc4ccc(C)c(c4)CNC(=O)[C@H](CCc4ccccc4)NC2=O)C3)CCN(C(=O)OC(C)(C)C)CC1] | tert-butyl 4-ethyl-4- (((9S,12S)-5⁴-methyl- 8,11,14-trioxo-9- phenethyl-4-oxa-7,10-diaza- 1(3,1)-piperidina-5(1,3)- benzenacyclotetradecaphane- 12-yl)carbamoyl)piperidine-1- carboxylate | 745.4 C₄₂H₅₉N₅O₇ | 746.7 |
| 150 | 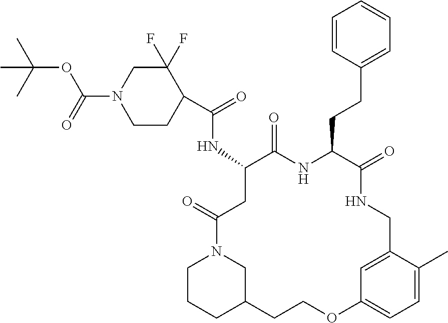 [SMILES: Cc1ccc2cc1CNC(=O)[C@H](CCc1ccccc1)NC(=O)[C@@H](NC(=O)C1CCN(C(=O)OC(C)(C)C)CC1(F)F)CC(=O)N1CCCC(CCO2)C1] | tert-butyl 3,3-difluoro-4- (((9S,12S)-5⁴-methyl-8,11,14- trioxo-9-phenethyl-4-oxa-7,10- diaza-1(3,1)-piperidina-5(1,3)- benzenacyclotetradecaphane- 12-yl)carbamoyl)piperidine-1- carboxylate | 753.4 C₄₀H₅₃F₂N₅O₇ | 754.6 |
| 153 | 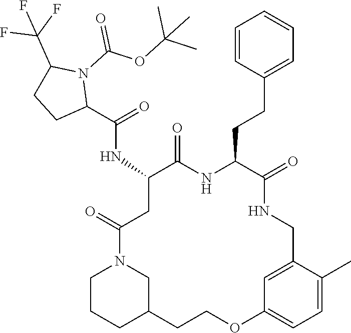 [SMILES: Cc1ccc2cc1CNC(=O)[C@H](CCc1ccccc1)NC(=O)[C@@H](NC(=O)C1CCC(C(F)(F)F)N1C(=O)OC(C)(C)C)CC(=O)N1CCCC(CCO2)C1] | tert-butyl 2-(((9S,12S)- 5⁴-methyl-8,11,14- trioxo-9-phenethyl-4-oxa- 7,10-diaza-1(3,1)-piperidina- 5(1,3)- benzenacyclotetradecaphane- 12-yl)carbamoyl)-5- (trifluoromethyl)pyrrolidine-1- carboxylate | 771.4 C₄₀H₅₂F₃N₅O₇ | 772.5 |
| 154 | 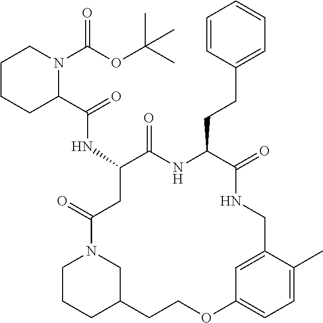 [SMILES: Cc1ccc2cc1CNC(=O)[C@H](CCc1ccccc1)NC(=O)[C@@H](NC(=O)C1CCCCN1C(=O)OC(C)(C)C)CC(=O)N1CCCC(CCO2)C1] | tert-butyl 2-(((9S,12S)- 5⁴-methyl-8,11,14- trioxo-9-phenethyl-4-oxa- 7,10-diaza-1(3,1)-piperidina- 5(1,3)- benzenacyclotetradecaphane- 12-yl)carbamoyl)piperidine-1- carboxylate | 717.4 C₄₀H₅₅N₅O₇ | 718.5 |
| 155 | 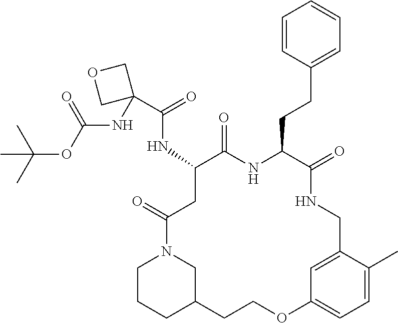 [SMILES: Cc1ccc2cc1CNC(=O)[C@H](CCc1ccccc1)NC(=O)[C@@H](NC(=O)C1(NC(=O)OC(C)(C)C)COC1)CC(=O)N1CCCC(CCO2)C1] | tert-butyl (3-(((9S,12S)- 5⁴-methyl-8,11,14- trioxo-9-phenethyl-4-oxa- 7,10-diaza-1(3,1)-piperidina- 5(1,3)- benzenacyclotetradecaphane- 12-yl)carbamoyl)oxetan-3- yl)carbamate | 705.4 C₃₈H₅₁N₅O₈ | 706.4 |
| 156 | 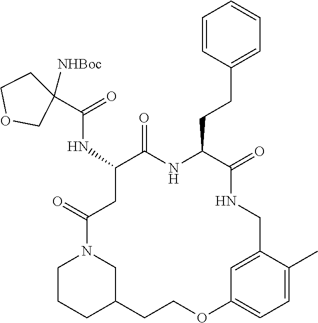 [SMILES: Cc1ccc2cc1CNC(=O)[C@H](CCc1ccccc1)NC(=O)[C@@H](NC(=O)C1(NC(=O)OC(C)(C)C)CCOC1)CC(=O)N1CCCC(CCO2)C1] | tert-butyl (3-(((9S,12S)- 5⁴-methyl-8,11,14- trioxo-9-phenethyl-4-oxa- 7,10-diaza-1(3,1)-piperidina- 5(1,3)- benzenacyclotetradecaphane- 12-yl)carbamoyl) tetrahydrofuran-3- yl)carbamate | 719.4 C₃₉H₅₃N₅O₈ | 720.5 |
| 161 | 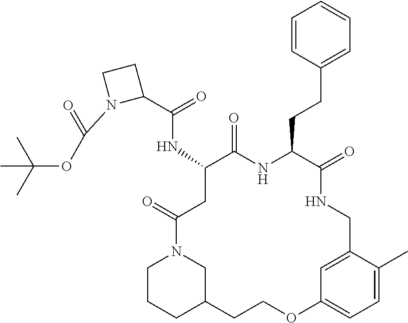 [SMILES: Cc1ccc2cc1CNC(=O)[C@H](CCc1ccccc1)NC(=O)[C@@H](NC(=O)C1CCN1C(=O)OC(C)(C)C)CC(=O)N1CCCC(CCO2)C1] | tert-butyl 2-(((9S,12S)- 5⁴-methyl-8,11,14- trioxo-9-phenethyl-4-oxa- 7,10-diaza-1(3,1)-piperidina- 5(1,3)- benzenacyclotetradecaphane- 12-yl)carbamoyl)azetidine-1- carboxylate | 689.4 C₃₈H₅₁N₅O₇ | 690.6 |
| 178 | 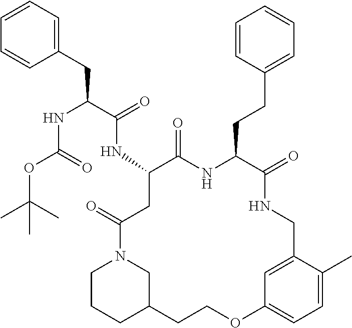 [SMILES: Cc1ccc2cc1CNC(=O)[C@H](CCc1ccccc1)NC(=O)[C@@H](NC(=O)[C@H](Cc1ccccc1)NC(=O)OC(C)(C)C)CC(=O)N1CCCC(CCO2)C1] | tert-butyl ((2S)-1- (((9S,12S)-5⁴-methyl- 8,11,14-trioxo-9- phenethyl-4-oxa-7,10-diaza- 1(3,1)-piperidina-5(1,3)- benzenacyclotetradecaphane- 12-yl)amino)-1-oxo-3- phenylpropan- 2-yl)carbamate | 753.4 C₄₃H₅₅N₅O₇ | 654.4 [M + H − Boc]+ |
| 387 | 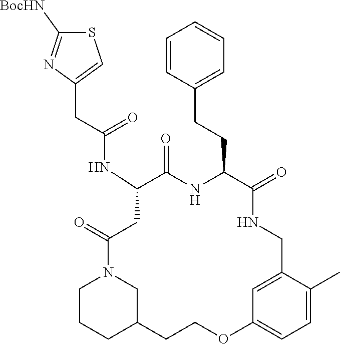 [SMILES: Cc1ccc2cc1CNC(=O)[C@H](CCc1ccccc1)NC(=O)[C@@H](NC(=O)Cc1csc(NC(=O)OC(C)(C)C)n1)CC(=O)N1CCCC(CCO2)C1] | tert-butyl (4-(2-(((9S,12S)-5⁴- methyl-8,11,14-trioxo-9- phenethyl-4-oxa-7,10-diaza- 1(3,1)-piperidina-5(1,3)- benzenacyclotetradecaphane- 12-yl)amino)-2-oxoethyl) thiazol-2- yl)carbamate | 746.3 C₃₉H₅₀N₆O₇S | 747.1 |
| 388 | 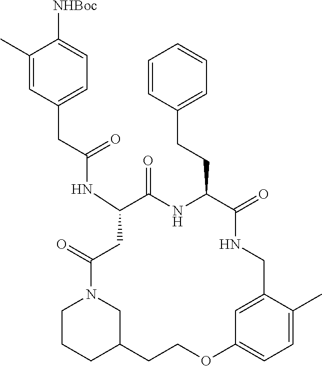 | tert-butyl (2-methyl-4-(2- (((9S,12S)-5⁴-methyl-8,11,14- trioxo-9-phenethyl-4-oxa-7,10- diaza-1(3,1)-piperidina-5(1,3)- benzenacyclotetradecaphane- 12-yl)amino)-2- oxoethyl)phenyl)carbamate | 753.4 C₄₃H₅₅N₅O₇ | 754.4 |
| 389 | 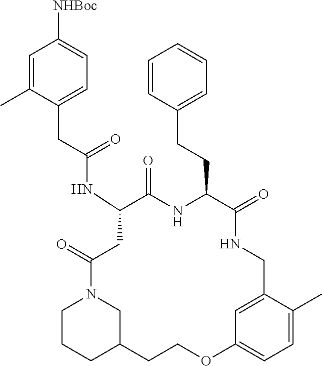 | tert-butyl (3-methyl-4-(2- (((9S,12S)-5⁴-methyl-8,11,14- trioxo-9-phenethyl-4-oxa-7,10- diaza-1(3,1)-piperidina-5(1,3)- benzenacyclotetradecaphane- 12-yl)amino)-2- oxoethyl)phenyl)carbamate | 753.4 C₄₃H₅₅N₅O₇ | 754.4 |
| 390 | 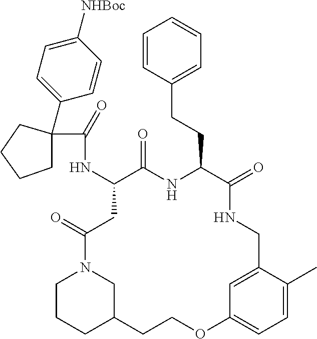 [SMILES: Cc1ccc2cc1CNC(=O)[C@H](CCc1ccccc1)NC(=O)[C@@H](NC(=O)C1(c3ccc(NC(=O)OC(C)(C)C)cc3)CCCC1)CC(=O)N1CCCC(CCO2)C1] | tert-butyl (4-(1-(((9S,12S)-5⁴- methyl-8,11,14-trioxo-9- phenethyl-4-oxa-7,10-diaza- 1(3,1)-piperidina-5(1,3)- benzenacyclotetradecaphane- 12-yl)carbamoyl) cyclopentyl)phenyl) carbamate | 793.4 C₄₆H₅₉N₅O₇ | 794.4 |
| 391 | 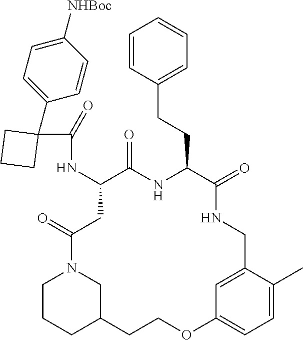 [SMILES: Cc1ccc2cc1CNC(=O)[C@H](CCc1ccccc1)NC(=O)[C@@H](NC(=O)C1(c3ccc(NC(=O)OC(C)(C)C)cc3)CCC1)CC(=O)N1CCCC(CCO2)C1] | tert-butyl (4-(1-(((9S,12S)-5⁴- methyl-8,11,14-trioxo-9- phenethyl-4-oxa-7,10-diaza- 1(3,1)-piperidina-5(1,3)- benzenacyclotetradecaphane- 12-yl)carbamoyl)cyclobutyl) phenyl)carbamate | 779.4 C₄₅H₅₇N₅O₇ | 780.4 |
| 392 | 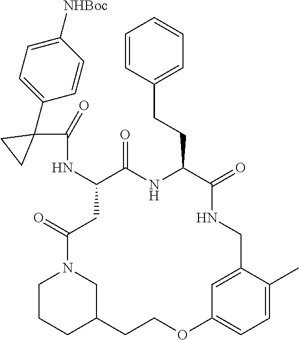 [SMILES: Cc1ccc2cc1CNC(=O)[C@H](CCc1ccccc1)NC(=O)[C@@H](NC(=O)C1(c3ccc(NC(=O)OC(C)(C)C)cc3)CC1)CC(=O)N1CCCC(CCO2)C1] | tert-butyl (4-(1-(((9S,12S)-5⁴- methyl-8,11,14-trioxo-9- phenethyl-4-oxa-7,10-diaza- 1(3,1)-piperidina-5(1,3)- benzenacyclotetradecaphane- 12-yl)carbamoyl) cyclopropyl)phenyl) carbamate | 765.4 C₄₄H₅₅N₅O₇ | 766.4 |
| 393 | 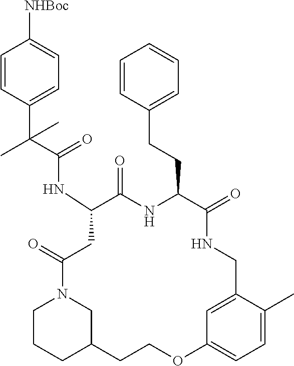 [SMILES: Cc1ccc2cc1CNC(=O)[C@H](CCc1ccccc1)NC(=O)[C@@H](NC(=O)C(C)(C)c1ccc(NC(=O)OC(C)(C)C)cc1)CC(=O)N1CCCC(CCO2)C1] | tert-butyl (4-(2-methyl-1- (((9S,12S)-5⁴-methyl-8,11,14- trioxo-9-phenethyl-4-oxa-7,10- diaza-1(3,1)-piperidina-5(1,3)- benzenacyclotetradecaphane- 12-yl)amino)-1-oxopropan-2- yl)phenyl)carbamate | 767.4 C₄₄H₅₇N₅O₇ | 768.4 |
| 394 | 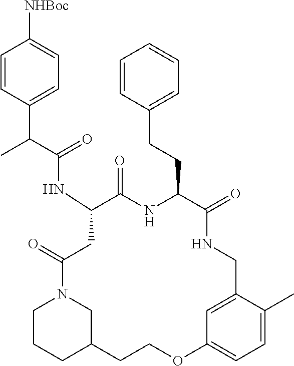 [SMILES: Cc1ccc2cc1CNC(=O)[C@H](CCc1ccccc1)NC(=O)[C@@H](NC(=O)C(C)c1ccc(NC(=O)OC(C)(C)C)cc1)CC(=O)N1CCCC(CCO2)C1] | 2-(4-aminophenyl)-N- ((9S,12S)-5⁴-methyl- 8,11,14-trioxo-9- phenethyl-4-oxa-7,10-diaza- 1(3,1)-piperidina-5(1,3)- benzenacyclotetradecaphane- 12-yl)propanamide | 653.4 C₃₈H₄₇N₅O₅ | 654.4 |
| 395 | 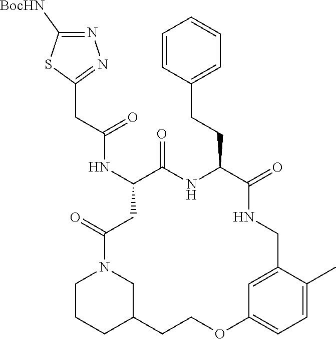 [SMILES: Cc1ccc2cc1CNC(=O)[C@H](CCc1ccccc1)NC(=O)[C@@H](NC(=O)Cc1nnc(NC(=O)OC(C)(C)C)s1)CC(=O)N1CCCC(CCO2)C1] | tert-butyl (5-(2-(((9S,12S)-5⁴- methyl-8,11,14-trioxo-9- phenethyl-4-oxa-7,10-diaza- 1(3,1)-piperidina-5(1,3)- benzenacyclotetradecaphane- 12-yl)amino)-2-oxoethyl)- 1,3,4-thiadiazol-2- yl)carbamate | 747.3 C₃₈H₄₉N₇O₇S | 748.4 |
| 396 | 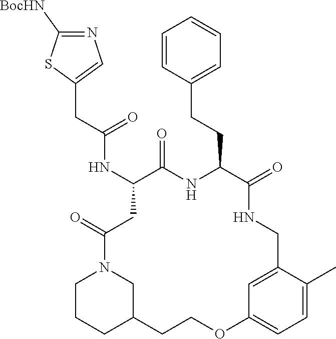 [SMILES: Cc1ccc2cc1CNC(=O)[C@H](CCc1ccccc1)NC(=O)[C@@H](NC(=O)Cc1cnc(NC(=O)OC(C)(C)C)s1)CC(=O)N1CCCC(CCO2)C1] | tert-butyl (5-(2-(((9S,12S)-5⁴- methyl-8,11,14-trioxo-9- phenethyl-4-oxa-7,10-diaza- 1(3,1)-piperidina-5(1,3)- benzenacyclotetradecaphane- 12-yl)amino)-2-oxoethyl) thiazol-2-yl)carbamate | 746.3 C₃₉H₅₀N₆O₇S | 747.4 |
| 419 | 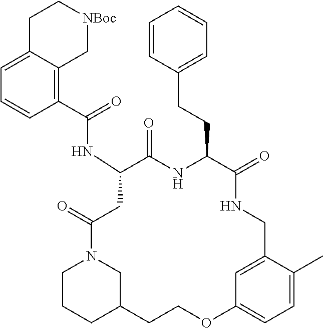 [SMILES: Cc1ccc2cc1CNC(=O)[C@H](CCc1ccccc1)NC(=O)[C@@H](NC(=O)c1cccc3c1CN(C(=O)OC(C)(C)C)CC3)CC(=O)N1CCCC(CCO2)C1] | tert-butyl 8-(((9S,12S)- 5⁴-methyl-8,11,14- trioxo-9-phenethyl-4-oxa- 7,10-diaza-1(3,1)-piperidina- 5(1,3)- benzenacyclotetradecaphane- 12-yl)carbamoyl)-3,4- dihydroisoquinoline-2(1H)- carboxylate | 765.4 C₄₄H₅₅N₅O₇ | 766.4 |
| 420 | 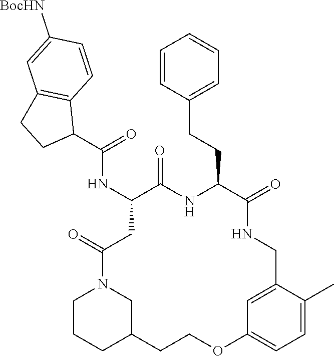 [SMILES: Cc1ccc2cc1CNC(=O)[C@H](CCc1ccccc1)NC(=O)[C@@H](NC(=O)C1CCc3cc(NC(=O)OC(C)(C)C)ccc31)CC(=O)N1CCCC(CCO2)C1] | tert-butyl (1-(((9S,12S)- 5⁴-methyl-8,11,14-trioxo- 9-phenethyl-4-oxa- 7,10-diaza-1(3,1)-piperidina- 5(1,3)- benzenacyclotetradecaphane- 12-yl)carbamoyl)-2,3- dihydro-1H- inden-5-yl)carbamate | 765.4 C₄₄H₅₅N₅O₇ | 766.3 |
| 421 | 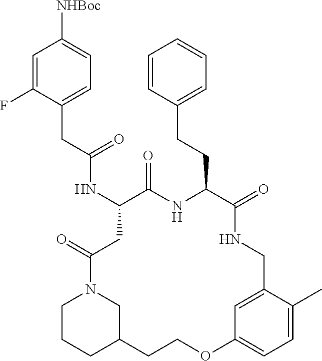 [SMILES: Cc1ccc2cc1CNC(=O)[C@H](CCc1ccccc1)NC(=O)[C@@H](NC(=O)Cc1ccc(NC(=O)OC(C)(C)C)cc1F)CC(=O)N1CCCC(CCO2)C1] | tert-butyl (3-fluoro-4-(2- (((9S,12S)-5⁴-methyl-8,11,14- trioxo-9-phenethyl-4-oxa-7,10- diaza-1(3,1)-piperidina-5(1,3)- benzenacyclotetradecaphane- 12-yl)amino)-2- oxoethyl)phenyl)carbamate | 757.4 C₄₂H₅₂FN₅O₇ | 758.3 |
| 422 | 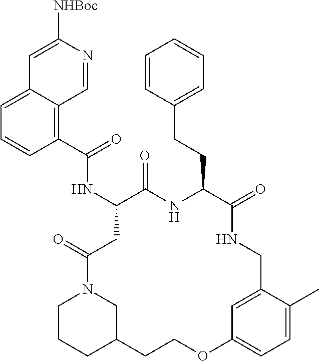 [SMILES: Cc1ccc2cc1CNC(=O)[C@H](CCc1ccccc1)NC(=O)[C@@H](NC(=O)c1cccc3cc(NC(=O)OC(C)(C)C)ncc13)CC(=O)N1CCCC(CCO2)C1] | tert-butyl (8-(((9S,12S)- 5⁴-methyl-8,11,14-trioxo- 9-phenethyl-4-oxa- 7,10-diaza-1(3,1)-piperidina- 5(1,3)- benzenacyclotetradecaphane- 12-yl)carbamoyl)isoquinolin- 3-yl)carbamate | 776.4 C₄₄H₅₂N₆O₇ | 777.3 |
| 423 | 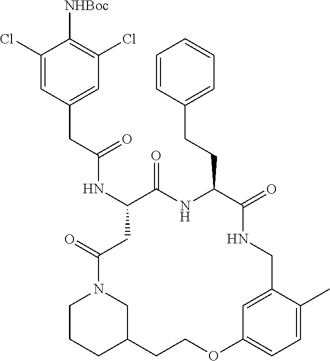 [SMILES: Cc1ccc2cc1CNC(=O)[C@H](CCc1ccccc1)NC(=O)[C@@H](NC(=O)Cc1cc(Cl)c(NC(=O)OC(C)(C)C)c(Cl)c1)CC(=O)N1CCCC(CCO2)C1] | tert-butyl (2,6-dichloro-4-(2- (((9S,12S)-5⁴-methyl-8,11,14- trioxo-9-phenethyl-4-oxa-7,10- diaza-1(3,1)-piperidina-5(1,3)- benzenacyclotetradecaphane- 12-yl)amino)-2- oxoethyl)phenyl)carbamate | 807.3 C₄₂H₅₁Cl₂N₅O₇ | 708.3 [M + H − Boc]+ |
| 429 | 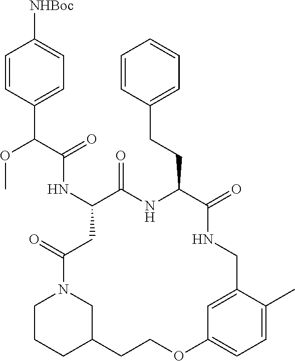 [SMILES: COC(C(=O)N[C@H]1CC(=O)N2CCCC(CCOc3ccc(C)c(c3)CNC(=O)[C@H](CCc3ccccc3)NC1=O)C2)c1ccc(NC(=O)OC(C)(C)C)cc1] | tert-butyl (4-(1-methoxy-2- (((9S,12S)-5⁴-methyl-8,11,14- trioxo-9-phenethyl-4-oxa-7,10- diaza-1(3,1)-piperidina-5(1,3)- benzenacyclotetradecaphane- 12-yl)amino)-2- oxoethyl)phenyl)carbamate | 769.4 C₄₃H₅₅N₅O₈ | 770.4 |
| 440 | 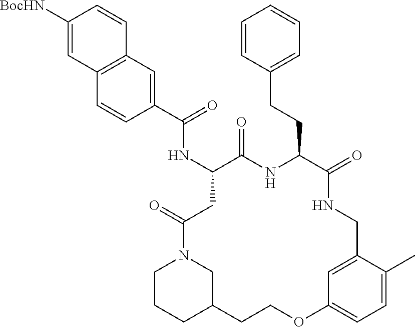 [SMILES: Cc1ccc2cc1CNC(=O)[C@H](CCc1ccccc1)NC(=O)[C@@H](NC(=O)c1ccc3cc(NC(=O)OC(C)(C)C)ccc3c1)CC(=O)N1CCCC(CCO2)C1] | tert-butyl (6-(((9S,12S)- 5⁴-methyl-8,11,14-trioxo- 9-phenethyl-4-oxa- 7,10-diaza-1(3,1)-piperidina- 5(1,3)- benzenacyclotetradecaphane- 12-yl)carbamoyl)naphthalen- 2-yl)carbamate | 775.4 C₄₅H₅₃N₅O₇ | 775.4 |
| 441 | 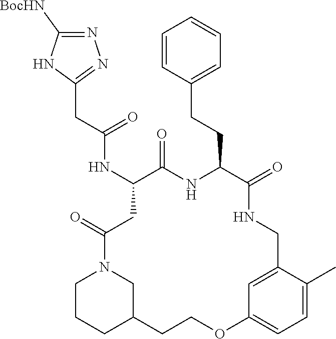 [SMILES: Cc1ccc2cc1CNC(=O)[C@H](CCc1ccccc1)NC(=O)[C@@H](NC(=O)Cc1nnc(NC(=O)OC(C)(C)C)[nH]1)CC(=O)N1CCCC(CCO2)C1] | tert-butyl (5-(2-(((9S,12S)-5⁴- methyl-8,11,14-trioxo-9- phenethyl-4-oxa-7,10-diaza- 1(3,1)-piperidina-5(1,3)- benzenacyclotetradecaphane- 12-yl)amino)-2-oxoethyl)- 4H-1,2,4- triazol-3-yl)carbamate | 730.4 C₃₈H₅₀N₈O₇ | 731.4 |
| 442 | 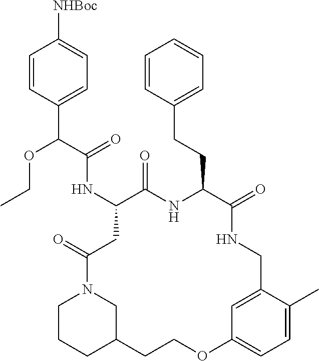 [SMILES: CCOC(C(=O)N[C@H]1CC(=O)N2CCCC(CCOc3ccc(C)c(c3)CNC(=O)[C@H](CCc3ccccc3)NC1=O)C2)c1ccc(NC(=O)OC(C)(C)C)cc1] | tert-butyl (4-(1-ethoxy-2- (((9S,12S)-5⁴-methyl-8,11,14- trioxo-9-phenethyl-4-oxa-7,10- diaza-1(3,1)-piperidina-5(1,3)- benzenacyclotetradecaphane- 12-yl)amino)-2- oxoethyl)phenyl)carbamate | 783.4 C₄₄H₅₇N₅O₈ | 784.5 |

### Example A-13:_Removal of Boc-Protecting Groups to Give Basic Macrocycles

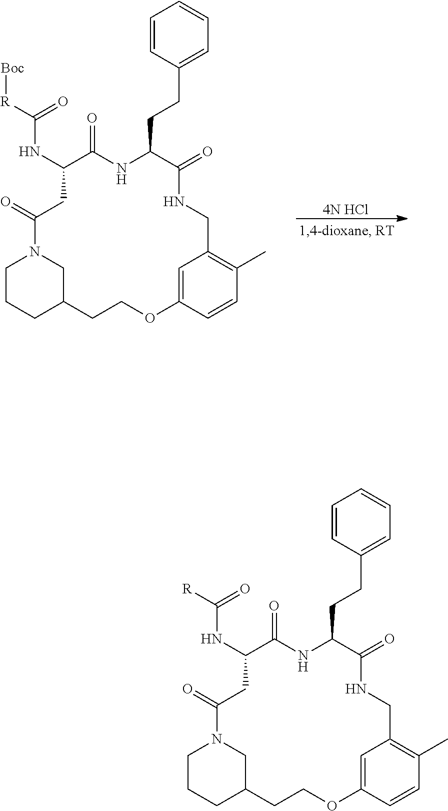

Boc protecting groups were cleaved from the compound according to general procedure D stirring compounds in 4-10 ml 4 N HCl/1,4-dioxane at room temperature. After deprotection was complete solvent was removed and the crude product was purified by reversed phase HPLC.

TABLE A-7

| Cpd | From | | | Exact | [M + H]⁺ |
|---|---|---|---|---|---|
| no | cpd | structure | name | mass | found |
| 126 | 121 | 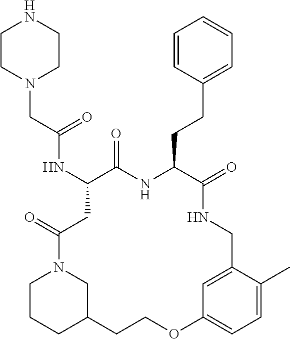 [SMILES: Cc1ccc2cc1CNC(=O)[C@H](CCc1ccccc1)NC(=O)[C@@H](NC(=O)CN1CCNCC1)CC(=O)N1CCCC(CCO2)C1] | N-((9S,12S)-5⁴-methyl-8,11,14- trioxo-9-phenethyl-4-oxa-7,10- diaza-1(3,1)-piperidina-5(1,3)- benzenacyclotetradecaphane- 12-yl)-2-(piperazin-1- yl)acetamide | 632.4 C₃₅H₄₈N₆O₅ | 633.5 |
| 127 | 122 | 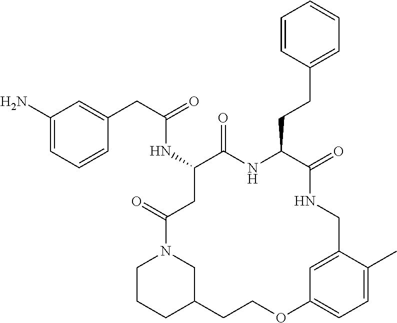 [SMILES: Cc1ccc2cc1CNC(=O)[C@H](CCc1ccccc1)NC(=O)[C@@H](NC(=O)Cc1cccc(N)c1)CC(=O)N1CCCC(CCO2)C1] | 2-(3-aminophenyl)-N- ((9S,12S)-5⁴-methyl-8,11,14- trioxo-9-phenethyl-4-oxa-7,10- diaza-1(3,1)-piperidina-5(1,3)- benzenacyclotetradecaphane- 12-yl)acetamide | 639.3 C₃₇H₄₅N₅O₅ | 640.5 |
| 128 | 123 | 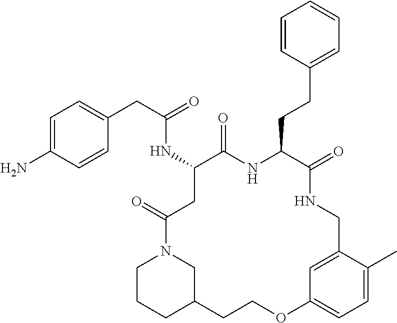 [SMILES: Cc1ccc2cc1CNC(=O)[C@H](CCc1ccccc1)NC(=O)[C@@H](NC(=O)Cc1ccc(N)cc1)CC(=O)N1CCCC(CCO2)C1] | 2-(4-aminophenyl)-N- ((9S,12S)-5⁴-methyl-8,11,14- trioxo-9-phenethyl-4-oxa-7,10- diaza-1(3,1)-piperidina-5(1,3)- benzenacyclotetradecaphane- 12-yl)acetamide | 639.3 C₃₇H₄₅N₅O₅ | 640.5 |
| 129 | 124 | 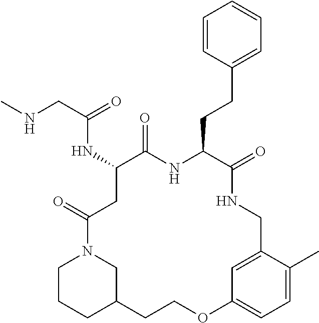 | N-((9S,12S)-5⁴-methyl-8,11,14- trioxo-9-phenethyl-4-oxa-7,10- diaza-1(3,1)-piperidina-5(1,3)- benzenacyclotetradecaphane- 12-yl)-2- (methylamino)acetamide | 577.3 C₃₂H₄₃N₅O₅ | 578.5 |
| 130 | 125 | 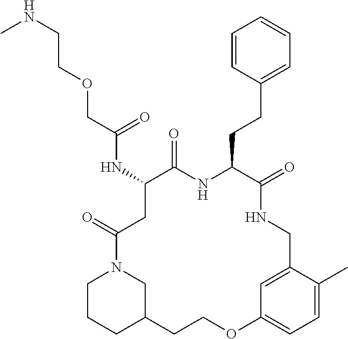 | N-((9S,12S)-5⁴-methyl-8,11,14- trioxo-9-phenethyl-4-oxa-7,10- diaza-1(3,1)-piperidina-5(1,3)- benzenacyclotetradecaphane- 12-yl)-2-(2- (methylamino)ethoxy)acetamide | 621.4 C₃₄H₄₇N₅O₆ | 622.6 |
| 134 | 131 | 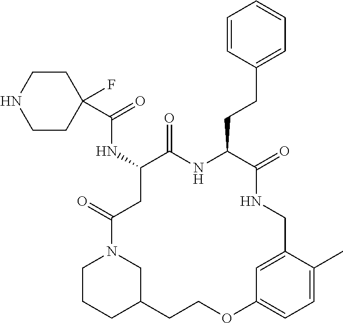 [SMILES: Cc1ccc2cc1CNC(=O)[C@H](CCc1ccccc1)NC(=O)[C@@H](NC(=O)C1(F)CCNCC1)CC(=O)N1CCCC(CCO2)C1] | 4-fluoro-N-((9S,12S)-5⁴- methyl-8,11,14-trioxo-9- phenethyl-4-oxa-7,10-diaza- 1(3,1)-piperidina-5(1,3)- benzenacyclotetradecaphane- 12-yl)piperidine-4-carboxamide | 635.3 C₃₅H₄₆FN₅O₅ | 636.5 |
| 135 | 132 | 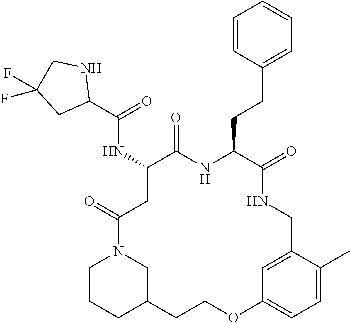 [SMILES: Cc1ccc2cc1CNC(=O)[C@H](CCc1ccccc1)NC(=O)[C@@H](NC(=O)C1CC(F)(F)CN1)CC(=O)N1CCCC(CCO2)C1] | (2S)-4,4-difluoro-N-((9S,12S)- 5⁴-methyl-8,11,14-trioxo-9- phenethyl-4-oxa-7,10-diaza- 1(3,1)-piperidina-5(1,3)- benzenacyclotetradecaphane- 12-yl)pyrrolidine-2- carboxamide | 639.3 C₃₄H₄₃F₂N₅O₅ | 640.4 |
| 136 | 133 | 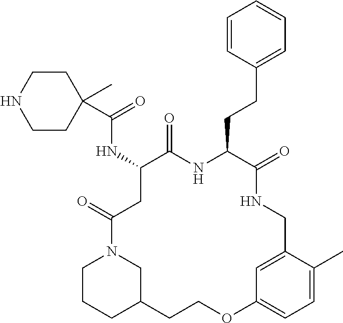 [SMILES: Cc1ccc2cc1CNC(=O)[C@H](CCc1ccccc1)NC(=O)[C@@H](NC(=O)C1(C)CCNCC1)CC(=O)N1CCCC(CCO2)C1] | 4-methyl-N-((9S,12S)-5⁴- methyl-8,11,14-trioxo-9- phenethyl-4-oxa-7,10-diaza- 1(3,1)-piperidina-5(1,3)- benzenacyclotetradecaphane- 12-yl)piperidine-4-carboxamide | 631.4 C₃₆H₄₉N₅O₅ | 632.5 |
| 151 | 149 | 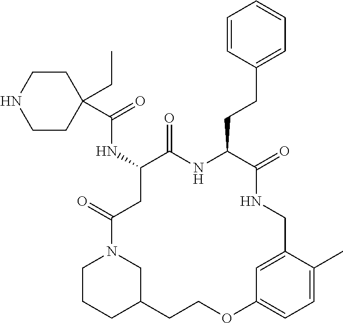 [SMILES: CCC1(C(=O)N[C@H]2CC(=O)N3CCCC(CCOc4ccc(C)c(c4)CNC(=O)[C@H](CCc4ccccc4)NC2=O)C3)CCNCC1] | 4-ethyl-N-((9S,12S)-5⁴-methyl- 8,11,14-trioxo-9-phenethyl-4- oxa-7,10-diaza-1(3,1)- piperidina-5(1,3)- benzenacyclotetradecaphane- 12-yl)piperidine-4-carboxamide | 645.4 C₃₇H₅₁N₅O₅ | 646.5 |
| 152 | 150 | 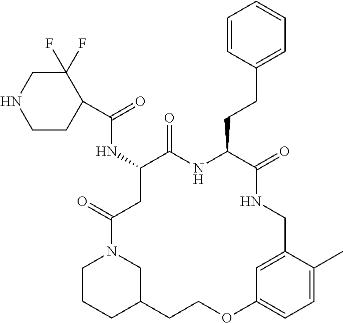 [SMILES: Cc1ccc2cc1CNC(=O)[C@H](CCc1ccccc1)NC(=O)[C@@H](NC(=O)C1CCNCC1(F)F)CC(=O)N1CCCC(CCO2)C1] | 3,3-difluoro-N-((9S,12S)-5⁴- methyl-8,11,14-trioxo-9- phenethyl-4-oxa-7,10-diaza- 1(3,1)-piperidina-5(1,3)- benzenacyclotetradecaphane- 12-yl)piperidine-4-carboxamide | 653.3 C₃₅H₄₅F₂N₅O₅ | 654.4 |
| 160 | 161 | 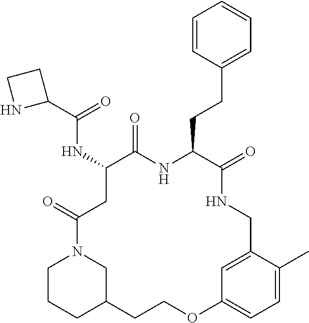 [SMILES: Cc1ccc2cc1CNC(=O)[C@H](CCc1ccccc1)NC(=O)[C@@H](NC(=O)C1CCN1)CC(=O)N1CCCC(CCO2)C1] | (2R)-N-((9S,12S)-5⁴-methyl- 8,11,14-trioxo-9-phenethyl-4- oxa-7,10-diaza-1(3,1)- piperidina-5(1,3)- benzenacyclotetradecaphane- 12-yl)azetidine-2-carboxamide | 589.74 C₃₃H₄₃N₅O₅ | — |
| 157 | 153 | 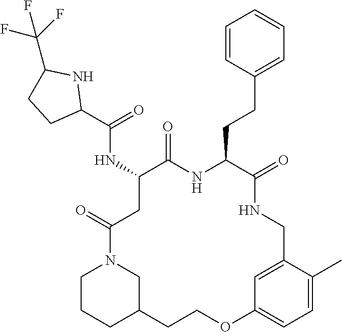 [SMILES: Cc1ccc2cc1CNC(=O)[C@H](CCc1ccccc1)NC(=O)[C@@H](NC(=O)C1CCC(C(F)(F)F)N1)CC(=O)N1CCCC(CCO2)C1] | N-((9S,12S)-5⁴-methyl-8,11,14- trioxo-9-phenethyl-4-oxa-7,10- diaza-1(3,1)-piperidina-5(1,3)- benzenacyclotetradecaphane- 12-yl)-5- (trifluoromethyl)pyrrolidine-2- carboxamide | 671.3 C₃₅H₄₄F₃N₅O₅ | 672.5 |
| 158 | 154 | 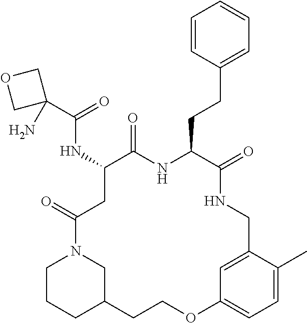 [SMILES: Cc1ccc2cc1CNC(=O)[C@H](CCc1ccccc1)NC(=O)[C@@H](NC(=O)C1(N)COC1)CC(=O)N1CCCC(CCO2)C1] | 3-amino-N-((9S,12S)-5⁴- methyl-8,11,14-trioxo-9- phenethyl-4-oxa-7,10-diaza- 1(3,1)-piperidina-5(1,3)- benzenacyclotetradecaphane- 12-yl)oxetane-3-carboxamide | 605.3 C₃₃H₄₃N₅O₆ | 606.5 |
| 159 | 155 | 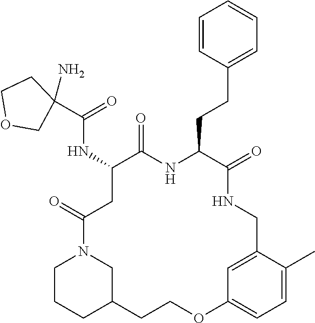 [SMILES: Cc1ccc2cc1CNC(=O)[C@H](CCc1ccccc1)NC(=O)[C@@H](NC(=O)C1(N)CCOC1)CC(=O)N1CCCC(CCO2)C1] | 3-amino-N-((9S,12S)-5⁴- methyl-8,11,14-trioxo-9- phenethyl-4-oxa-7,10-diaza- 1(3,1)-piperidina-5(1,3)- benzenacyclotetradecaphane- 12-yl)tetrahydrofuran-3- carboxamide | 619.3 C₃₄H₄₅N₅O₆ | 620.5 |
| 179 | 178 | 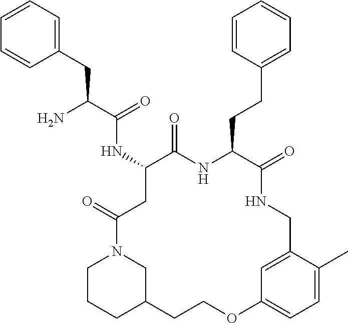 [SMILES: Cc1ccc2cc1CNC(=O)[C@H](CCc1ccccc1)NC(=O)[C@@H](NC(=O)[C@@H](N)Cc1ccccc1)CC(=O)N1CCCC(CCO2)C1] | (2R)-2-amino-N-((9S,12S)-5⁴- methyl-8,11,14-trioxo-9- phenethyl-4-oxa-7,10-diaza- 1(3,1)-piperidina-5(1,3)- benzenacyclotetradecaphane- 12-yl)-3-phenylpropanamide- | 653.4 C₃₈H₄₇N₅O₅ | 654.4 |
| 302 | 387 | 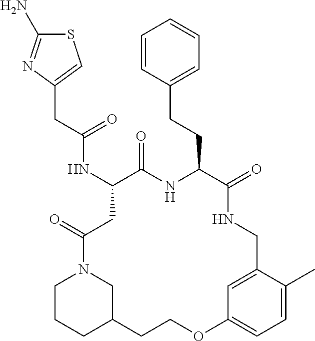 [SMILES: Cc1ccc2cc1CNC(=O)[C@H](CCc1ccccc1)NC(=O)[C@@H](NC(=O)Cc1csc(N)n1)CC(=O)N1CCCC(CCO2)C1] | 2-(2-aminothiazol-4-yl)-N- ((9S,12S)-5⁴-methyl-8,11,14- trioxo-9-phenethyl-4-oxa-7,10- diaza-1(3,1)-piperidina-5(1,3)- benzenacyclotetradecaphane- 12-yl)acetamide | 646.3 C₃₄H₄₂N₆O₅S | 647.7 |
| 303 | 388 | 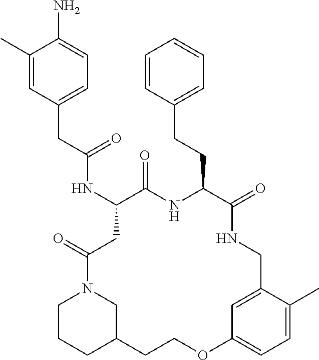 | 2-(4-amino-3-methylphenyl)-N- ((9S,12S)-5⁴-methyl-8,11,14- trioxo-9-phenethyl-4-oxa-7,10- diaza-1(3,1)-piperidina-5(1,3)- benzenacyclotetradecaphane- 12-yl)acetamide | 653.4 C₃₈H₄₇N₅O₅ | 654.6 |
| 304 | 389 | 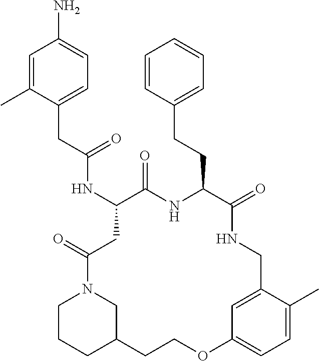 | 2-(4-amino-2-methylphenyl)-N- ((9S,12S)-5⁴-methyl-8,11,14- trioxo-9-phenethyl-4-oxa-7,10- diaza-1(3,1)-piperidina-5(1,3)- benzenacyclotetradecaphane- 12-yl)acetamide | 653.4 C₃₈H₄₇N₅O₅ | 654.4 |
| 305 | 390 | 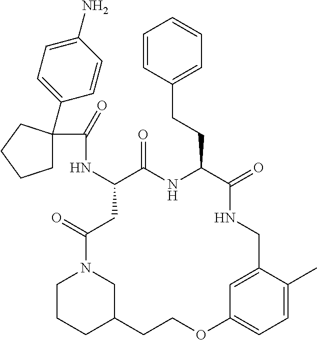 [SMILES: Cc1ccc2cc1CNC(=O)[C@H](CCc1ccccc1)NC(=O)[C@@H](NC(=O)C1(c3ccc(N)cc3)CCCC1)CC(=O)N1CCCC(CCO2)C1] | 1-(4-aminophenyl)-N- ((9S,12S)-5⁴-methyl-8,11,14- trioxo-9-phenethyl-4-oxa-7,10- diaza-1(3,1)-piperidina-5(1,3)- benzenacyclotetradecaphane- 12-yl)cyclopentane-1- carboxamide | 693.4 C₄₁H₅₁N₅O₅ | 694.5 |
| 306 | 391 | 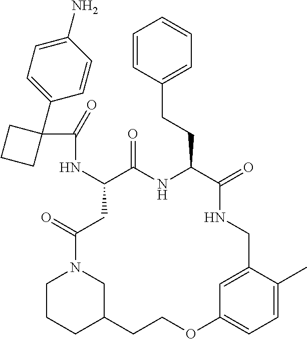 [SMILES: Cc1ccc2cc1CNC(=O)[C@H](CCc1ccccc1)NC(=O)[C@@H](NC(=O)C1(c3ccc(N)cc3)CCC1)CC(=O)N1CCCC(CCO2)C1] | 1-(4-aminophenyl)-N- ((9S,12S)-5⁴-methyl-8,11,14- trioxo-9-phenethyl-4-oxa-7,10- diaza-1(3,1)-piperidina-5(1,3)- benzenacyclotetradecaphane- 12-yl)cyclobutane-1- carboxamide | 679.4 C₄₀H₄₉N₅O₅ | 680.4 |
| 307 | 392 | 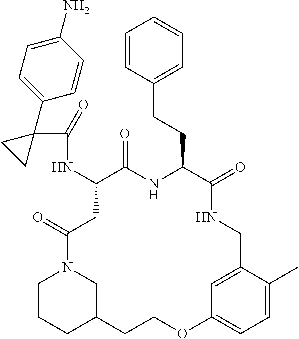 [SMILES: Cc1ccc2cc1CNC(=O)[C@H](CCc1ccccc1)NC(=O)[C@@H](NC(=O)C1(c3ccc(N)cc3)CC1)CC(=O)N1CCCC(CCO2)C1] | 1-(4-aminophenyl)-N- ((9S,12S)-5⁴-methyl-8,11,14- trioxo-9-phenethyl-4-oxa-7,10- diaza-1(3,1)-piperidina-5(1,3)- benzenacyclotetradecaphane- 12-yl)cyclopropane-1- carboxamide | 665.4 C₃₉H₄₇N₅O₅ | 666.4 |
| 308 | 393 | 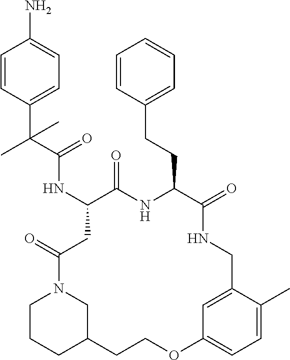 [SMILES: Cc1ccc2cc1CNC(=O)[C@H](CCc1ccccc1)NC(=O)[C@@H](NC(=O)C(C)(C)c1ccc(N)cc1)CC(=O)N1CCCC(CCO2)C1] | 2-(4-aminophenyl)-2-methyl-N- ((9S,12S)-5⁴-methyl-8,11,14- trioxo-9-phenethyl-4-oxa-7,10- diaza-1(3,1)-piperidina-5(1,3)- benzenacyclotetradecaphane- 12-yl)propanamide | 667.4 C₃₉H₄₉N₅O₅ | 668.5 |
| 309 | 394 | 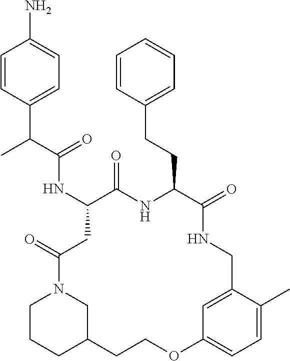 [SMILES: Cc1ccc2cc1CNC(=O)[C@H](CCc1ccccc1)NC(=O)[C@@H](NC(=O)C(C)c1ccc(N)cc1)CC(=O)N1CCCC(CCO2)C1] | 2-(4-aminophenyl)-N- ((9S,12S)-5⁴-methyl-8,11,14- trioxo-9-phenethyl-4-oxa-7,10- diaza-1(3,1)-piperidina-5(1,3)- benzenacyclotetradecaphane- 12-yl)propanamide | 653.4 C₃₈H₄₇N₅O₅ | 654.4 |
| 310 | 395 | 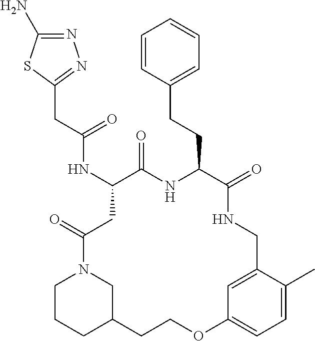 [SMILES: Cc1ccc2cc1CNC(=O)[C@H](CCc1ccccc1)NC(=O)[C@@H](NC(=O)Cc1nnc(N)s1)CC(=O)N1CCCC(CCO2)C1] | 2-(5-amino-1,3,4-thiadiazol-2- yl)-N-((9S,12S)-54-methyl- 8,11,14-trioxo-9-phenethyl-4- oxa-7,10-diaza-1(3,1)- piperidina-5(1,3)- benzenacyclotetradecaphane- 12-yl)acetamide | 647.3 C₃₃H₄₁N₇O₅S | 648.3 |
| 311 | 396 | 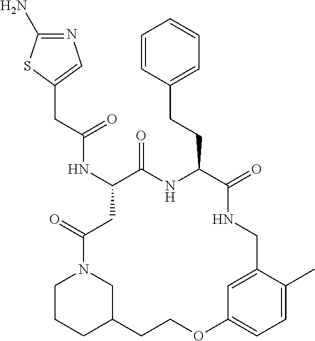 [SMILES: Cc1ccc2cc1CNC(=O)[C@H](CCc1ccccc1)NC(=O)[C@@H](NC(=O)Cc1cnc(N)s1)CC(=O)N1CCCC(CCO2)C1] | 2-(2-aminothiazol-5-yl)-N- ((9S,12S)-5⁴-methyl-8,11,14- trioxo-9-phenethyl-4-oxa-7,10- diaza-1(3,1)-piperidina-5(1,3)- benzenacyclotetradecaphane- 12-yl)acetamide | 646.3 C₃₄H₄₂N₆O₅S | 647.4 |
| 316 | 419 | 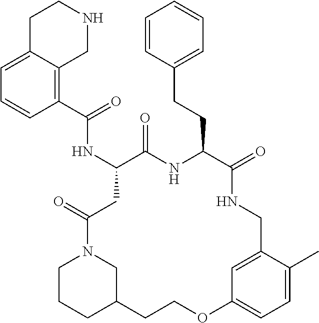 [SMILES: Cc1ccc2cc1CNC(=O)[C@H](CCc1ccccc1)NC(=O)[C@@H](NC(=O)c1cccc3c1CNCC3)CC(=O)N1CCCC(CCO2)C1] | N-((9S,12S)-5⁴-methyl-8,11,14- trioxo-9-phenethyl-4-oxa-7,10- diaza-1(3,1)-piperidina-5(1,3)- benzenacyclotetradecaphane- 12-yl)-1,2,3,4- tetrahydroisoquinoline-5- carboxamide | 665.4 C₃₉H₄₇N₅O₅ | 666.4 |
| 317 | 420 | 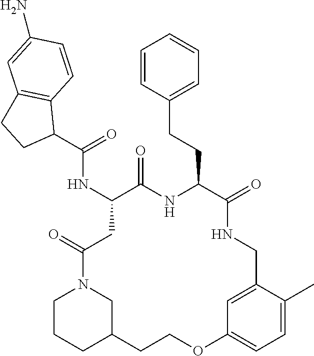 [SMILES: Cc1ccc2cc1CNC(=O)[C@H](CCc1ccccc1)NC(=O)[C@@H](NC(=O)C1CCc3cc(N)ccc31)CC(=O)N1CCCC(CCO2)C1] | 5-amino-N-((9S,12S)-5⁴- methyl-8,11,14-trioxo-9- phenethyl-4-oxa-7,10-diaza- 1(3,1)-piperidina-5(1,3)- benzenacyclotetradecaphane- 12-yl)-2,3-dihydro-1H-indene- 1-carboxamide | 665.4 C₃₉H₄₇N₅O₅ | 666.5 |
| 318 | 421 | 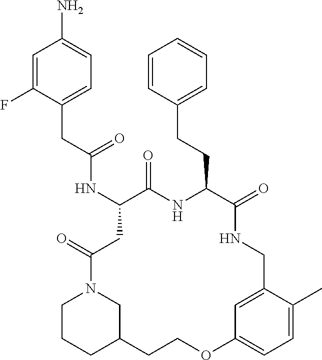 [SMILES: Cc1ccc2cc1CNC(=O)[C@H](CCc1ccccc1)NC(=O)[C@@H](NC(=O)Cc1ccc(N)cc1F)CC(=O)N1CCCC(CCO2)C1] | 2-(4-amino-2-fluorophenyl)-N- ((9S,12S)-5⁴-methyl-8,11,14- trioxo-9-phenethyl-4-oxa-7,10- diaza-1(3,1)-piperidina-5(1,3)- benzenacyclotetradecaphane- 12-yl)acetamide | 657.3 C₃₇H₄₄FN₅O₅ | 658.4 |
| 319 | 422 | 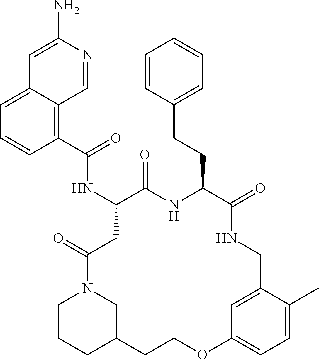 [SMILES: Cc1ccc2cc1CNC(=O)[C@H](CCc1ccccc1)NC(=O)[C@@H](NC(=O)c1cccc3cc(N)ncc13)CC(=O)N1CCCC(CCO2)C1] | 3-amino-N-((9S,12S)-5⁴- methyl-8,11,14-trioxo-9- phenethyl-4-oxa-7,10-diaza- 1(3,1)-piperidina-5(1,3)- benzenacyclotetradecaphane- 12-yl)isoquinoline-8- carboxamide | 676.3 C₃₉H₄₄N₆O₅ | 677.4 |
| 320 | 423 | 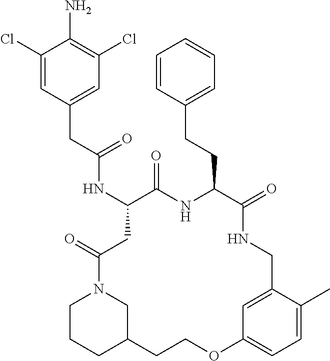 [SMILES: Cc1ccc2cc1CNC(=O)[C@H](CCc1ccccc1)NC(=O)[C@@H](NC(=O)Cc1cc(Cl)c(N)c(Cl)c1)CC(=O)N1CCCC(CCO2)C1] | 2-(4-amino-3,5- dichlorophenyl)-N-((9S,12S)- 5⁴-methyl-8,11,14-trioxo-9- phenethyl-4-oxa-7,10-diaza- 1(3,1)-piperidina-5(1,3)- benzenacyclotetradecaphane- 12-yl)acetamide | 707.3 C₃₇H₄₃Cl₂N₅O₅ | 708.3 |
| 326 | 429 | 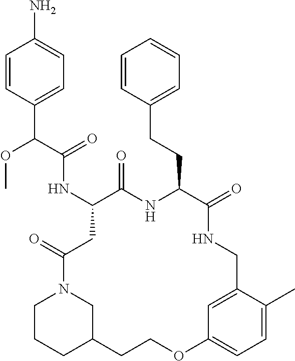 [SMILES: COC(C(=O)N[C@H]1CC(=O)N2CCCC(CCOc3ccc(C)c(c3)CNC(=O)[C@H](CCc3ccccc3)NC1=O)C2)c1ccc(N)cc1] | 2-(4-aminophenyl)-2-methoxy- N-((9S,12S)-5⁴-methyl-8,11,14- trioxo-9-phenethyl-4-oxa-7,10- diaza-1(3,1)-piperidina-5(1,3)- benzenacyclotetradecaphane- 12-yl)acetamide | 669.4 C₃₈H₄₇N₅O₆ | 670.4 |
| 339 | 440 | 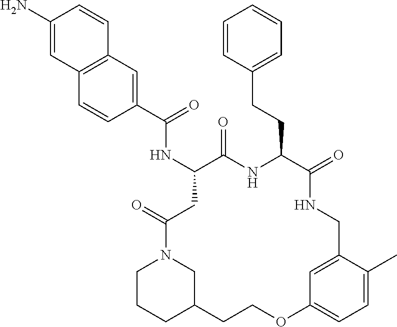 [SMILES: Cc1ccc2cc1CNC(=O)[C@H](CCc1ccccc1)NC(=O)[C@@H](NC(=O)c1ccc3cc(N)ccc3c1)CC(=O)N1CCCC(CCO2)C1] | 6-amino-N-((9S,12S)-5⁴- methyl-8,11,14-trioxo-9- phenethyl-4-oxa-7,10-diaza- 1(3,1)-piperidina-5(1,3)- benzenacyclotetradecaphane- 12-yl)-2-naphthamide | 675.3 C₄₀H₄₅N₅O₅ | 676.4 |
| 340 | 441 | 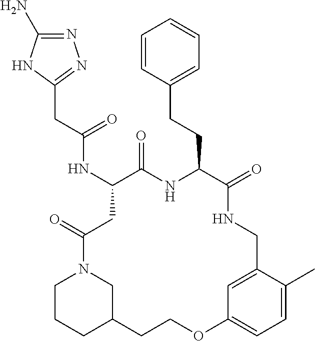 [SMILES: Cc1ccc2cc1CNC(=O)[C@H](CCc1ccccc1)NC(=O)[C@@H](NC(=O)Cc1nnc(N)[nH]1)CC(=O)N1CCCC(CCO2)C1] | 2-(5-amino-1H-1,2,4-triazol-3- yl)-N-((9S,12S)-5⁴-methyl- 8,11,14-trioxo-9-phenethyl-4- oxa-7,10-diaza-1(3,1)- piperidina-5(1,3)- benzenacyclotetradecaphane- 12-yl)acetamide | 630.3 C₃₃H₄₂N₈O₅ | 631.4 |
| 345 | 442 | 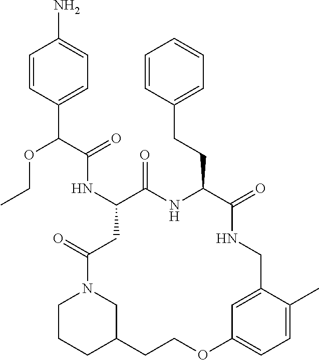 [SMILES: CCOC(C(=O)N[C@H]1CC(=O)N2CCCC(CCOc3ccc(C)c(c3)CNC(=O)[C@H](CCc3ccccc3)NC1=O)C2)c1ccc(N)cc1] | 2-(4-aminophenyl)-2-ethoxy-N- ((9S,12S)-5⁴-methyl-8,11,14- trioxo-9-phenethyl-4-oxa-7,10- diaza-1(3,1)-piperidina-5(1,3)- benzenacyclotetradecaphane- 12-yl)acetamide | 683.4 C₃₉H₄₉N₅O₆ | 684.4 |

### Example A-14: Preparation of Intermediate Compounds

### (S)-tert-butyl 4-acetamido-5-(((S)-1-ethoxy-1-oxo-4-phenylbutan-2-yl)amino)-5-oxopentanoate (137)

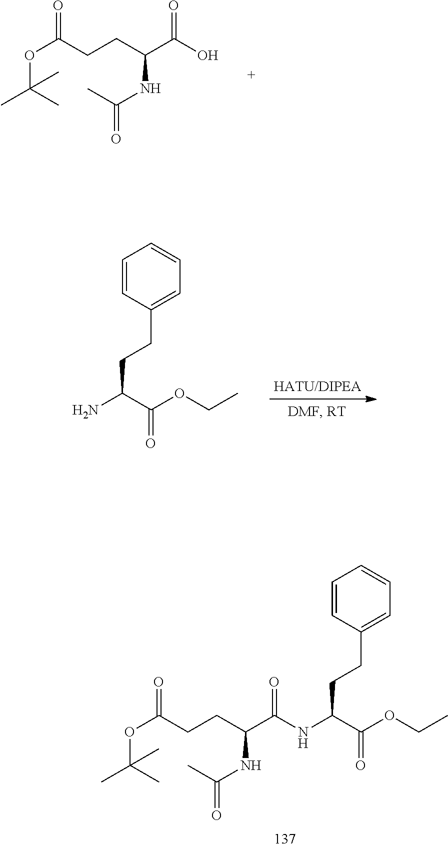 [SMILES: CC(=O)N[C@@H](CCC(=O)OC(C)(C)C)C(=O)O ; CCOC(=O)[C@@H](N)CCc1ccccc1 ; CCOC(=O)[C@H](CCc1ccccc1)NC(=O)[C@H](CCC(=O)OC(C)(C)C)NC(C)=O]

The amide bond can be formed according to general procedure B with Ac-Glu(OtBu)-OH and H-hPhe-OEt. Purification can be achieved by normal phase flash chromatography.

Formula: C₂₃H₃₄N₂O₆, exact mass: 434.2, found: 435.3 [M+H]⁺

### (S)-2-((S)-2-acetamido-5-(tert-butoxy)-5-oxopentanamido)-4-phenylbutanoic acid (138)

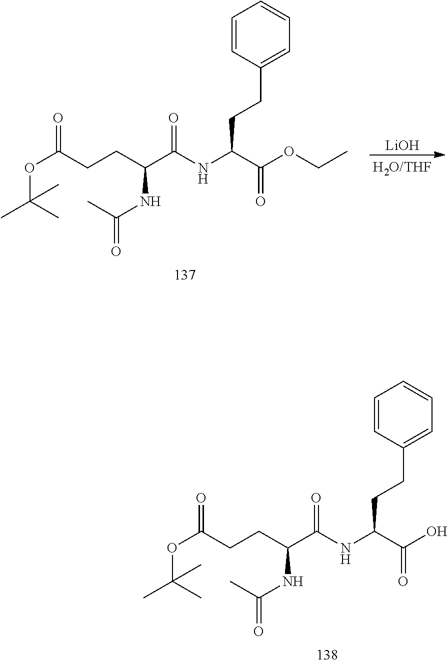

Ester 137 was dissolved in 3 ml THF. 0.5 ml water and 0.5 2 M LiOH aq. were added at room temperature. After complete saponification the mixture was acidified to pH 2 and extracted with ethyl acetate. The organic phase was dried over MgSO₄ and solvents were removed under reduced pressure. The residue was used in the next step without further purification.

Formula: C₂₁H₃₀N₂O₆, exact mass: 406.2, found: 407.5 [M+H]⁺

### tert-butyl 2-(2-(3-cyano-4-methylphenoxy)ethyl)morpholine-4-carboxylate (140)

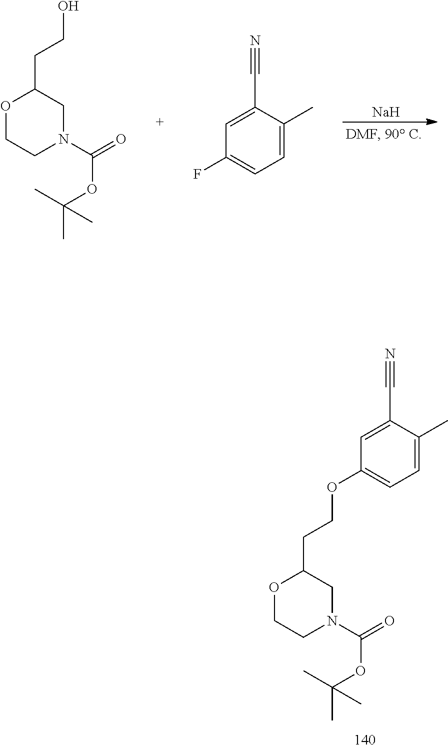

924 mg alcohol was deprotonated in DMF with 153 mg NaH at room temperature. 450 mg 5-fluoro-2-methylbenzonitrile were added and the mixture was heated to 90° C. until reaction was complete. Reaction mixture was diluted with ethyl acetate and washed with saturated NaHCO₃ solution and brine. After drying over MgSO₄ and removing the solvent under reduced pressure the residue was purified by normal phase column chromatography (silica, cyclohexane/ethyl acetate gradient).

Formula: C₁₉H₂₆N₂O₄, exact mass: 346.2, found: 247.3 [M+H]⁺

Building blocks in the following table were synthesized as exemplified by compound 140 utilizing appropriately substituted 3-fluorobenzonitriles and related boc-protected aminoalcohols.

TABLE A-8

| Cpd | | | Exact | [M + H]⁺ |
|---|---|---|---|---|
| no | structure | name | mass | found |
| 140 | 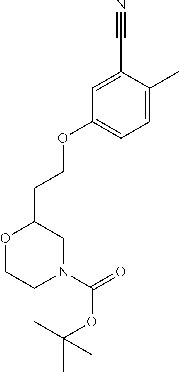 [SMILES: Cc1ccc(OCCC2CN(C(=O)OC(C)(C)C)CCO2)cc1C#N] | tert-butyl 2-(2-(3-cyano-4- methylphenoxy)ethyl)morpholine-4- carboxylate | 346.2 C₁₉H₂₆N₂O₄ | 247.3 [M + H − Boc]+ |
| 144 | 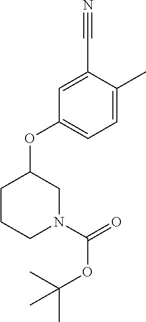 [SMILES: Cc1ccc(OC2CCCN(C(=O)OC(C)(C)C)C2)cc1C#N] | tert-butyl 3-(3-cyano-4- methylphenoxy)piperidine-1-carboxylate | 316.2 C₁₈H₂₄N₂O₃ | 217.1 [M + H − Boc]+ |
| 187 | 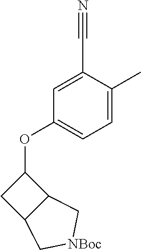 [SMILES: Cc1ccc(OC2CC3CN(C(=O)OC(C)(C)C)CC32)cc1C#N] | tert-butyl 6-(3-cyano-4-methylphenoxy)- 3-azabicyclo[3.2.0]heptane-3- carboxylate | 328.2 C₁₉H₂₄N₂O₃ | 229.3 [M + H − Boc]+ |
| 229 | 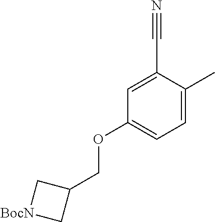 [SMILES: Cc1ccc(OCC2CN(C(=O)OC(C)(C)C)C2)cc1C#N] | tert-butyl 3-((3-cyano-4- methylphenoxy)methyl)azetidine-1- carboxylate | 302.2 C₁₇H₂₂N₂O₃ | |
| 413 | 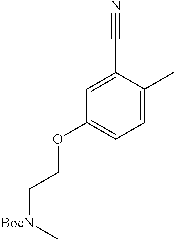 [SMILES: Cc1ccc(OCCN(C)C(=O)OC(C)(C)C)cc1C#N] | tert-butyl (2-(3-cyano-4- methylphenoxy)ethyl)(methyl)carbamate | 290.2 C₁₆H₂₂N₂O₃ | |
| 412 | 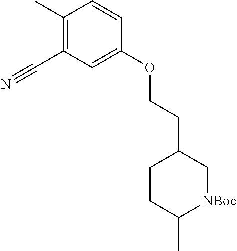 [SMILES: Cc1ccc(OCCC2CCC(C)N(C(=O)OC(C)(C)C)C2)cc1C#N] | tert-butyl 5-(2-(3-cyano-4- methylphenoxy)ethyl)-2- methylpiperidine-1-carboxylate | 358.2 C₂₁H₃₀N₂O₃ | |
| 414 | 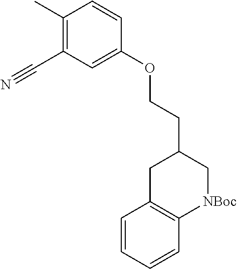 [SMILES: Cc1ccc(OCCC2Cc3ccccc3N(C(=O)OC(C)(C)C)C2)cc1C#N] | tert-butyl 3-(2-(3-cyano-4- methylphenoxy)ethyl)-3,4- dihydroquinoline-1(2H)-carboxylate | 392.2 C₂₄H₂₈N₂O₃ | |
| 415 | 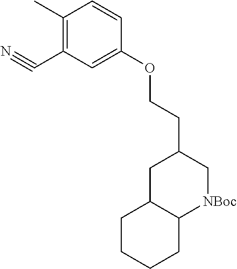 [SMILES: Cc1ccc(OCCC2CC3CCCCC3N(C(=O)OC(C)(C)C)C2)cc1C#N] | tert-butyl 3-(2-(3-cyano-4- methylphenoxy)ethyl) octahydroquinoline-1(2H)-carboxylate | 398.3 C₂₄H₃₄N₂O₃ | |
| 431 | 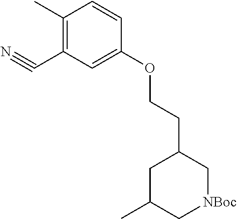 [SMILES: Cc1ccc(OCCC2CC(C)CN(C(=O)OC(C)(C)C)C2)cc1C#N] | tert-butyl 3-(2-(3-cyano-4- methylphenoxy)ethyl)-5- methylpiperidine-1-carboxylate | 358.2 C₂₁H₃₀N₂O₃ | 359.3 |
| 454 | 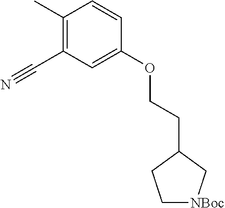 [SMILES: Cc1ccc(OCCC2CCN(C(=O)OC(C)(C)C)C2)cc1C#N] | tert-butyl 3-(2-(3-cyano-4- methylphenoxy)ethyl)pyrrolidine-1- carboxylate | 330.2 C₁₉H₂₆N₂O₃ | 331.4 |
| 453 | 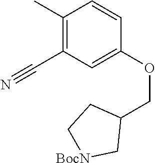 [SMILES: Cc1ccc(OCC2CCN(C(=O)OC(C)(C)C)C2)cc1C#N] | tert-butyl 3-((3-cyano-4- methylphenoxy)methyl)pyrrolidine-1- carboxylate | 316.2 C₁₈H₂₄N₂O₃ | 317.4 |
| 462 | 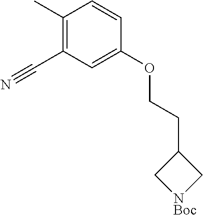 [SMILES: Cc1ccc(OCCC2CN(C(=O)OC(C)(C)C)C2)cc1C#N] | tert-butyl 3-(2-(3-cyano-4- methylphenoxy)ethyl)azetidine-1- carboxylate | 316.2 C₁₈H₂₄N₂O₃ | 317.3 |
| 466 | 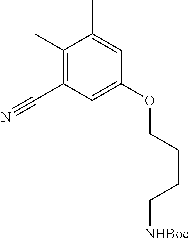 [SMILES: Cc1cc(OCCCCNC(=O)OC(C)(C)C)cc(C#N)c1C] | tert-butyl (4-(3-cyano-4,5- dimethylphenoxy)butyl)carbamate | 318.2 C₁₈H₂₆N₂O₃ | 319.4 |
| 573 | 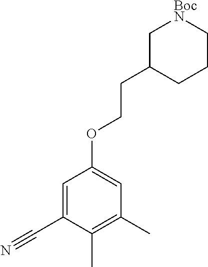 [SMILES: Cc1cc(OCCC2CCCN(C(=O)OC(C)(C)C)C2)cc(C#N)c1C] | tert-butyl 3-(2-(3-cyano-4,5- dimethylphenoxy)ethyl)piperidine-1- carboxylate | 358.2 C₂₁H₃₀N₂O₃ | |
| 577 | 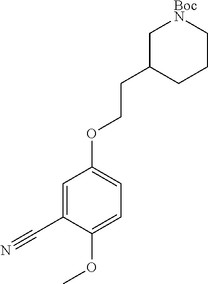 [SMILES: COc1ccc(OCCC2CCCN(C(=O)OC(C)(C)C)C2)cc1C#N] | tert-butyl 3-(2-(3-cyano-4- methoxyphenoxy)ethyl)piperidine-1- carboxylate | 360.2 C₂₀H₂₈N₂O₄ | |
| 578 | 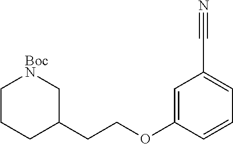 [SMILES: CC(C)(C)OC(=O)N1CCCC(CCOc2cccc(C#N)c2)C1] | tert-butyl 3-(2-(3- cyanophenoxy)ethyl)piperidine-1- carboxylate | 330.2 C₁₉H₂₆N₂O₃ | 331.3 |

### tert-butyl 2-(2-(3-(aminomethyl)-4-methylphenoxy)ethyl)morpholine-4-carboxylate (141)

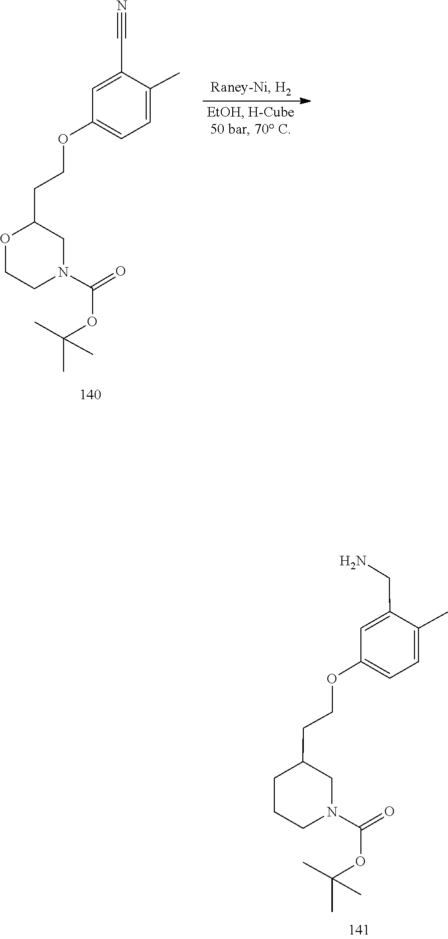 [SMILES: Cc1ccc(OCCC2CN(C(=O)OC(C)(C)C)CCO2)cc1C#N ; Cc1ccc(OCCC2CCCN(C(=O)OC(C)(C)C)C2)cc1CN]

1.15 g nitrile 140 were reduced was cleaved according to general procedure H using Raney-Ni catalyst cartridge, 50 bar at 70° C. in 70 ml ethanol. Volatiles were removed under reduced pressure and the residue was purified by normal phase column chromatography (silica, cyclohexane/ethyl acetate gradient). Building blocks in the following table were synthesized as exemplified by compound 141.

Formula: C₁₉H₃₀N₂O₄, exact mass: 350.2, found: 351.3 [M+H]⁺

TABLE A-9

| Cpd | From | | | Exact | [M + H]⁺ |
|---|---|---|---|---|---|
| no | cpd | structure | name | mass | found |
| 90 | 573 | 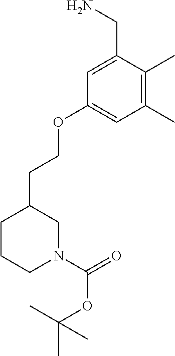 [SMILES: Cc1cc(OCCC2CCCN(C(=O)OC(C)(C)C)C2)cc(CN)c1C] | tert-butyl 3-(2-(3-(aminomethyl)-4,5- dimethylphenoxy)ethyl)piperidine-1- carboxylate | 362.3 C₂₁H₃₄ N₂O₃ | 363.3 |
| 141 | 140 | 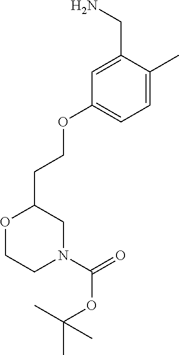 [SMILES: Cc1ccc(OCCC2CN(C(=O)OC(C)(C)C)CCO2)cc1CN] | tert-butyl 2-(2-(3-(aminomethyl)-4- methylphenoxy)ethyl) morpholine-4- carboxylate | 350.2 C₁₉H₃₀ N₂O₄ | 351.3 |
| 145 | 144 | 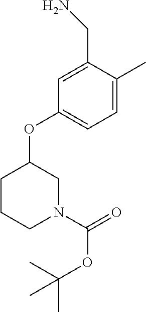 [SMILES: Cc1ccc(OC2CCCN(C(=O)OC(C)(C)C)C2)cc1CN] | tert-butyl 3-(3-(aminomethyl)-4- methylphenoxy)piperidine-1-carboxylate | 320.2 C₁₈H₂₈ N₂O₃ | 321.3 |
| 188 | 187 | 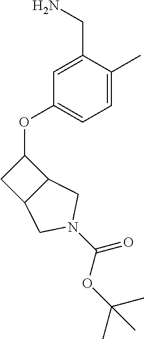 [SMILES: Cc1ccc(OC2CC3CN(C(=O)OC(C)(C)C)CC32)cc1CN] | tert-butyl 6-(3-(aminomethyl)-4- methylphenoxy)-3- azabicyclo[3.2.0]heptane-3-carboxylate | 332.2 C₁₉H₂₈ N₂O₃ | 333.3 |
| 222 | | 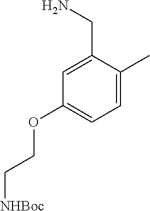 [SMILES: Cc1ccc(OCCNC(=O)OC(C)(C)C)cc1CN] | tert-butyl (2-(3-(aminomethyl)-4- methylphenoxy)ethyl)carbamate | 280.2 C₁₅H₂₄ N₂O₃ | 281.2 |
| 224 | | 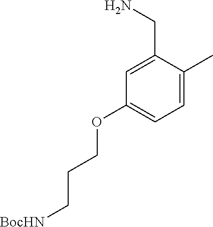 [SMILES: Cc1ccc(OCCCNC(=O)OC(C)(C)C)cc1CN] | tert-butyl (3-(3-(aminomethyl)-4- methylphenoxy)propyl)carbamate | 294.2 C₁₆H₂₆ N₂O₃ | 295.2 |
| 225 | | 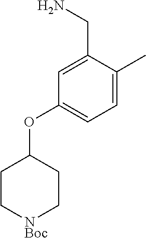 [SMILES: Cc1ccc(OC2CCN(C(=O)OC(C)(C)C)CC2)cc1CN] | tert-butyl 4-(3-(aminomethyl)-4- methylphenoxy)piperidine-1-carboxylate | 320.2 C₁₈H₂₈ N₂O₃ | 321.5 |
| 226 | | 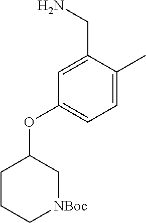 [SMILES: Cc1ccc(OC2CCCN(C(=O)OC(C)(C)C)C2)cc1CN] | tert-butyl 3-(3-(aminomethyl)-4- methylphenoxy)piperidine-1-carboxylate | 320.2 C₁₈H₂₈ N₂O₃ | 321.3 |
| 227 | | 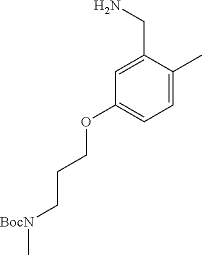 [SMILES: Cc1ccc(OCCCN(C)C(=O)OC(C)(C)C)cc1CN] | tert-butyl (3-(3-(aminomethyl)-4- methylphenoxy)propyl)(methyl)carbamate | 308.2 C₁₇H₂₈ N₂O₃ | 309.1 |
| 228 | | 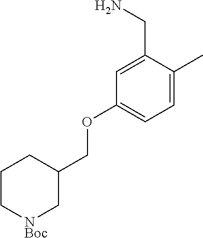 [SMILES: Cc1ccc(OCC2CCCN(C(=O)OC(C)(C)C)C2)cc1CN] | tert-butyl 3-((3-(aminomethyl)-4- methylphenoxy)methyl)piperidine-1- carboxylate | 334.2 C₁₉H₃₀ N₂O₃ | 335.3 |
| 230 | 229 | 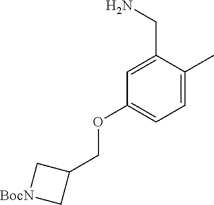 [SMILES: Cc1ccc(OCC2CN(C(=O)OC(C)(C)C)C2)cc1CN] | tert-butyl 3-((3-(aminomethyl)-4- methylphenoxy)methyl)azetidine-1- carboxylate | 306.2 C₁₇H₂₆ N₂O₃ | 307.3 |
| 231 | | 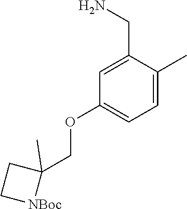 [SMILES: Cc1ccc(OCC2(C)CCN2C(=O)OC(C)(C)C)cc1CN] | tert-butyl 2-((3-(aminomethyl)-4- methylphenoxy)methyl)-2- methylazetidine-1-carboxylate | 320.2 C₁₈H₂₈ N₂O₃ | 321.2 |
| 232 | | 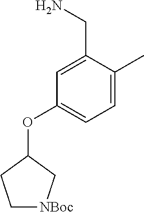 [SMILES: Cc1ccc(OC2CCN(C(=O)OC(C)(C)C)C2)cc1CN] | tert-butyl 3-(3-(aminomethyl)-4- methylphenoxy)pyrrolidine-1-carboxylate | 306.2 C₁₇H₂₆ N₂O₃ | 307.2 |
| 270 | | 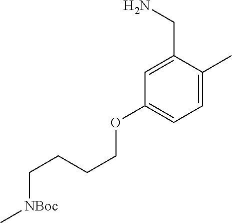 | tert-butyl (4-(3-(aminomethyl)-4- methylphenoxy)butyl)(methyl)carbamate | 322.2 C₁₈H₃₀ N₂O₃ | 323.2 |
| 271 | | 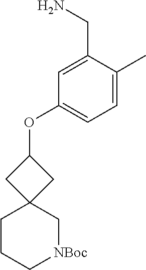 [SMILES: Cc1ccc(OC2CC3(CCCN(C(=O)OC(C)(C)C)C3)C2)cc1CN] | tert-butyl 2-(3-(aminomethyl)-4- methylphenoxy)-6-azaspiro[3.5]nonane-6- carboxylate | 360.2 C₂₁H₃₂ N₂O₃ | 361.1 |
| 399 | 413 | 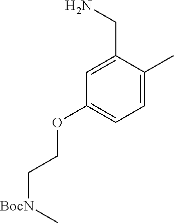 [SMILES: Cc1ccc(OCCN(C)C(=O)OC(C)(C)C)cc1CN] | tert-butyl (2-(3-(aminomethyl)-4- methylphenoxy)ethyl)(methyl)carbamate | 294.2 C₁₆H₂₆ N₂O₃ | — |
| 411 | 412 | 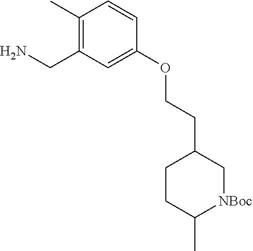 [SMILES: Cc1ccc(OCCC2CCC(C)N(C(=O)OC(C)(C)C)C2)cc1CN] | tert-butyl 5-(2-(3-(aminomethyl)-4- methylphenoxy)ethyl)-2- methylpiperidine-1-carboxylate | 362.3 C₂₁H₃₄ N₂O₃ | 363.3 |
| 416 | 414 | 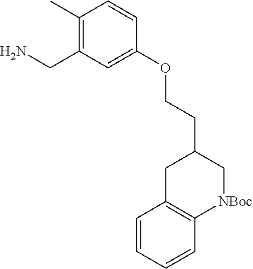 [SMILES: Cc1ccc(OCCC2Cc3ccccc3N(C(=O)OC(C)(C)C)C2)cc1CN] | tert-butyl 3-(2-(3-(aminomethyl)-4- methylphenoxy)ethyl)-3,4- dihydroquinoline-1(2H)-carboxylate | 396.2 C₂₄H₃₂ N₂O₃ | 397.3 |
| 417 | 415 | 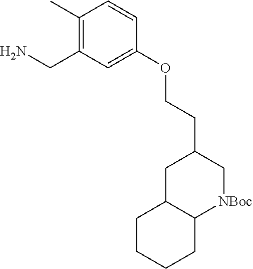 [SMILES: Cc1ccc(OCCC2CC3CCCCC3N(C(=O)OC(C)(C)C)C2)cc1CN] | tert-butyl 3-(2-(3-(aminomethyl)-4- methylphenoxy)ethyl)octahydroquinoline- 1(2H)-carboxylate | 402.3 C₂₄H₃₈ N₂O₃ | 403.4 |
| 426 | 454 | 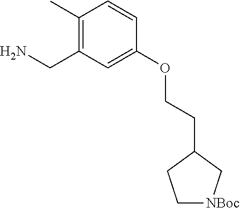 [SMILES: Cc1ccc(OCCC2CCN(C(=O)OC(C)(C)C)C2)cc1CN] | tert-butyl 3-(2-(3-(aminomethyl)-4- methylphenoxy)ethyl)pyrrolidine-1- carboxylate | 334.2 C₁₉H₃₀ N₂O₃ | 335.4 |
| 430 | 431 | 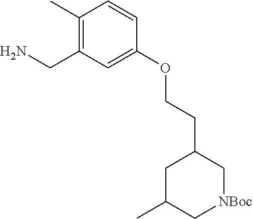 [SMILES: Cc1ccc(OCCC2CC(C)CN(C(=O)OC(C)(C)C)C2)cc1CN] | tert-butyl 3-(2-(3-(aminomethyl)-4- methylphenoxy)ethyl)-5- methylpiperidine-1-carboxylate | 362.3 C₂₁H₃₄ N₂O₃ | 363.4 |
| 455 | 453 | 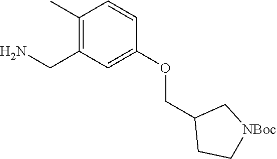 [SMILES: Cc1ccc(OCC2CCN(C(=O)OC(C)(C)C)C2)cc1CN] | tert-butyl 3-((3-(aminomethyl)-4- methylphenoxy)methyl)pyrrolidine-1- carboxylate | 320.2 C₁₈H₂₈ N₂O₃ | 321.3 |
| 463 | 462 | 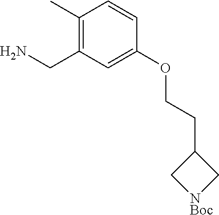 [SMILES: Cc1ccc(OCCC2CN(C(=O)OC(C)(C)C)C2)cc1CN] | tert-butyl 3-(2-(3-(aminomethyl)-4- methylphenoxy)ethyl)azetidine-1- carboxylate | 320.2 C₁₈H₂₈ N₂O₃ | 321.3 |
| 468 | 466 | 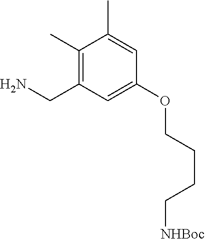 [SMILES: Cc1cc(OCCCCNC(=O)OC(C)(C)C)cc(CN)c1C] | tert-butyl (4-(3-(aminomethyl)-4,5- dimethylphenoxy)butyl)carbamate | 322.2 C₁₈H₃₀ N₂O₃ | 323.3 |
| 206 | 577 | 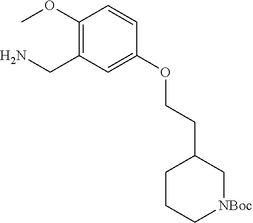 [SMILES: COc1ccc(OCCC2CCCN(C(=O)OC(C)(C)C)C2)cc1CN] | tert-butyl 3-(2-(3-(aminomethyl)-4- methoxyphenoxy)ethyl)piperidine-1- carboxylate | 364.2 C₂₀H₃₂ N₂O₄ | 365.0 |
| 579 | 578 | 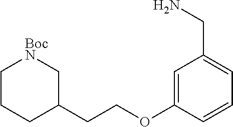 [SMILES: CC(C)(C)OC(=O)N1CCCC(CCOc2cccc(CN)c2)C1] | tert-butyl 3-(2-(3- (aminomethyl)phenoxy)ethyl)piperidine- 1-carboxylate | 334.2 C₁₉H₃₀ N₂O₃ | 335.3 |

### Example A-15: Preparation of Macrocyclic Compounds 143, and 148

### tert-butyl 2-(2-(3-(((S)-2-((S)-2-acetamido-5-(tert-butoxy)-5-oxopentanamido)-4-phenylbutanamido)methyl)-4-methylphenoxy)ethyl)morpholine-4-carboxylate (139)

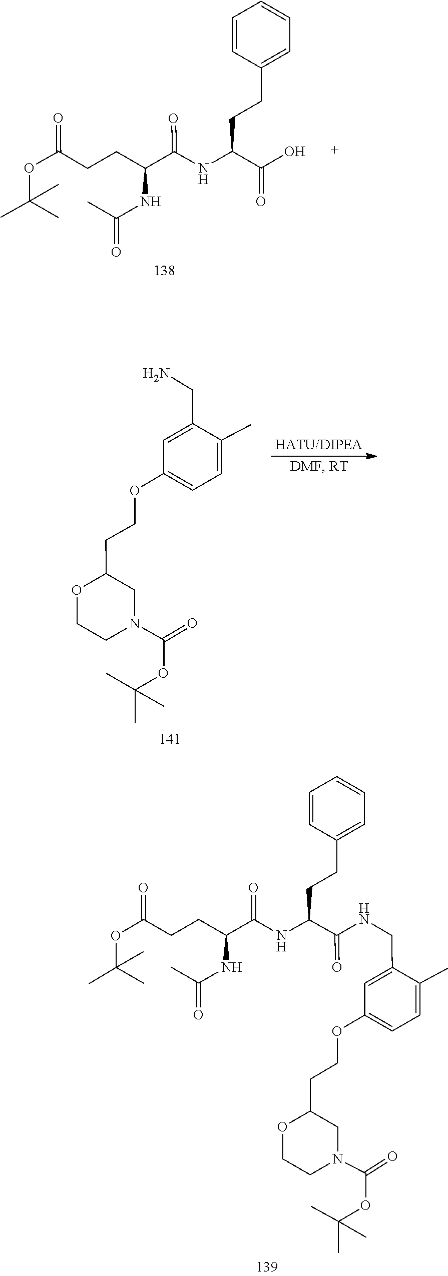 [SMILES: CC(=O)N[C@@H](CCC(=O)OC(C)(C)C)C(=O)N[C@@H](CCc1ccccc1)C(=O)O ; Cc1ccc(OCCC2CN(C(=O)OC(C)(C)C)CCO2)cc1CN ; CC(=O)N[C@@H](CCC(=O)OC(C)(C)C)C(=O)N[C@@H](CCc1ccccc1)C(=O)NCc1cc(OCCC2CN(C(=O)OC(C)(C)C)CCO2)ccc1C]

The amide bond was formed according to general procedure B with 96 mg amine 141 and 94 mg protected amino acid 138. Purification was achieved by normal phase flash chromatography. Building blocks in the following table were synthesized as exemplified by compound 139.

TABLE A-10

| Cpd | From | | | Exact | [M + H]⁺ |
|---|---|---|---|---|---|
| no | cpd | structure | name | mass | found |
| 139 | 141 | 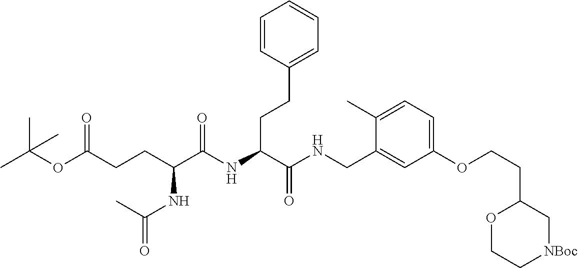 [SMILES: CC(=O)N[C@@H](CCC(=O)OC(C)(C)C)C(=O)N[C@@H](CCc1ccccc1)C(=O)NCc1cc(OCCC2CN(C(=O)OC(C)(C)C)CCO2)ccc1C] | tert-butyl 2- (2-(3-(((S)-2- ((S)-2-acetamido- 5-(tert- butoxy)-5- oxopentanamido)- 4- phenylbutanamido) methyl)- 4- methylphenoxy) ethyl)morpholine- 4-carboxylate | 738.4 C₄₀H₅₈ N₄O₉ | 739.5 |
| 146 | 145 | 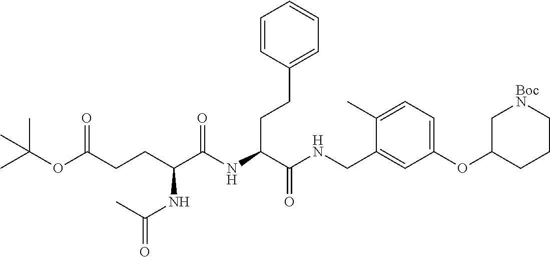 [SMILES: CC(=O)N[C@@H](CCC(=O)OC(C)(C)C)C(=O)N[C@@H](CCc1ccccc1)C(=O)NCc1cc(OC2CCCN(C(=O)OC(C)(C)C)C2)ccc1C] | tert-butyl 3- (3-(((S)-2-((S)-2- acetamido-5- (tert-butoxy)-5- oxopentanamido)- 4- phenylbutanamido) methyl)- 4- methylphenoxy) piperidine- 1-carboxylate | 708.4 C₃₉H₅₆ N₄O₈ | 709.6 |

### (4S)-4-acetamido-5-(((2S)-1-((2-methyl-5-(2-(morpholin-2-yl)ethoxy)benzyl)amino)-1-oxo-4-phenylbutan-2-yl)amino)-5-oxopentanoic acid (142)

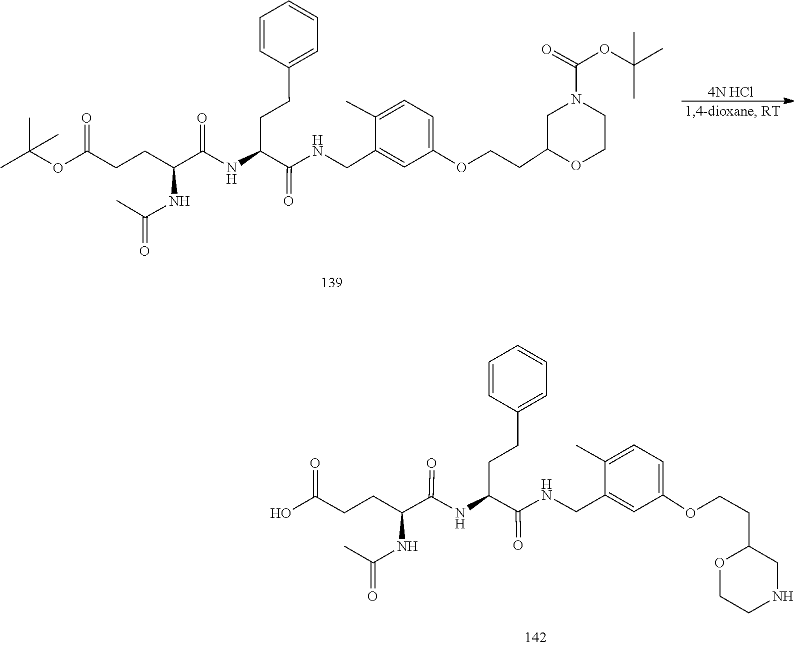

Protecting groups were cleaved from compound 139 according to general procedure D stirring compounds in 10 ml 4 N HCl/1,4-dioxane at room temperature. After deprotection was complete solvent was removed and the crude dissolved in acetonitrile and concentrated twice. Product used without further purification.

Building blocks in the following table were synthesized as exemplified by compound 142.

TABLE A-11

| Cpd | From | | | [M + H]⁺ | [M + H]⁺ |
|---|---|---|---|---|---|
| no | cpd | structure | name | calc. | found |
| 142 | 139 | 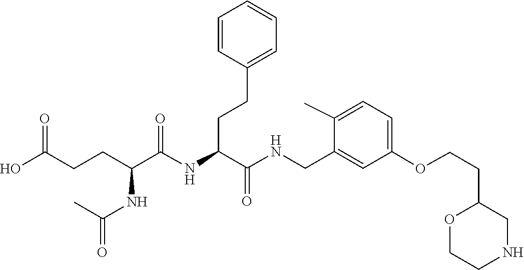 [SMILES: CC(=O)N[C@@H](CCC(=O)O)C(=O)N[C@@H](CCc1ccccc1)C(=O)NCc1cc(OCCC2CNCCO2)ccc1C] | (4S)-4- acetamido-5- (((2S)-1- ((2-methyl-5- (2-(morpholin-2- yl)ethoxy) benzyl)amino)- 1-oxo-4- phenylbutan-2- yl)amino)-5- oxopentanoic acid | 582.3 C₃₁H₄₂ N₄O₇ | 583.6 |
| 147 | 146 | 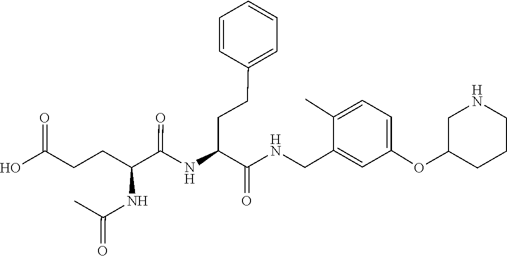 [SMILES: CC(=O)N[C@@H](CCC(=O)O)C(=O)N[C@@H](CCc1ccccc1)C(=O)NCc1cc(OC2CCCNC2)ccc1C] | (4S)-4- acetamido-5- (((2S)-1- ((2-methyl-5- (piperidin-3- yloxy)benzyl) amino)- 1-oxo-4- phenylbutan- 2-yl)amino)-5- oxopentanoic acid | 552.3 C₃₀H₄₀ N₄O₆ | 553.4 |

### N-((9S,12S)-5⁴-methyl-8,11,15-trioxo-9-phenethyl-4-oxa-7,10-diaza-1(2,4)-morpholina-5(1,3)-benzenacyclopentadecaphane-12-yl)acetamide (143)

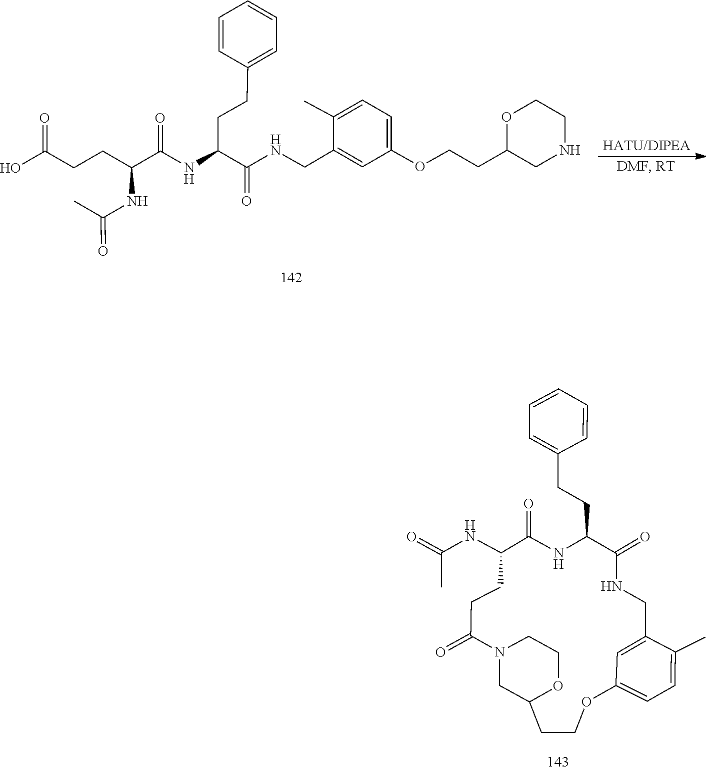 [SMILES: CC(=O)N[C@@H](CCC(=O)O)C(=O)N[C@@H](CCc1ccccc1)C(=O)NCc1cc(OCCC2CNCCO2)ccc1C ; CC(=O)N[C@H]1CCC(=O)N2CCOC(CCOc3ccc(C)c(c3)CNC(=O)[C@H](CCc3ccccc3)NC1=O)C2]

Macrocyclization was achieved according to general procedure A with 0.20 mmol amino acid 142. Reaction mixture was diluted with some methanol and purified via HPLC. Compounds in the following table were synthesized as exemplified by compound 143.

TABLE A-12

| Cpd | From | Scale | | | Exact | [M + H]⁺ |
|---|---|---|---|---|---|---|
| no | cpd | [nmol] | structure | name | mass | found |
| 143 | 142 | 200 | 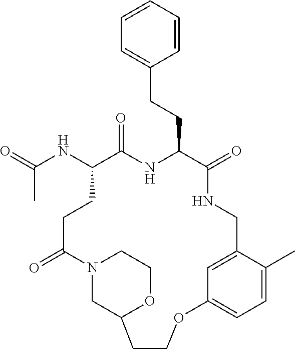 [SMILES: CC(=O)N[C@H]1CCC(=O)N2CCOC(CCOc3ccc(C)c(c3)CNC(=O)[C@H](CCc3ccccc3)NC1=O)C2] | N-((9S,12S)-5⁴-methyl- 8,11,15-trioxo-9-phenethyl- 4-oxa-7,10-diaza-1(2,4)- morpholina-5(1,3)- benzenacyclopentadecaphane- 12-yl)acetamide | 564.3 C₃₁H₄₀ N₄O₆ | 565.4 |
| 148 | 147 | 230 | 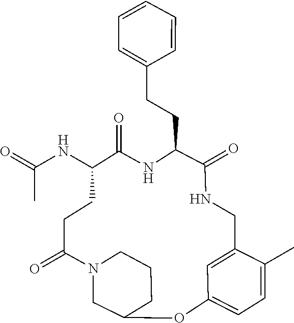 [SMILES: CC(=O)N[C@H]1CCC(=O)N2CCCC(C2)Oc2ccc(C)c(c2)CNC(=O)[C@H](CCc2ccccc2)NC1=O] | N-((7S,10S)-3⁴-methyl- 6,9,13-trioxo-7-phenethyl-2- oxa-5,8-diaza-1(3,1)- piperidina-3(1,3)- benzenacyclotridecaphane- 10-yl)acetamide | 534.3 C₃₀H₃₈ N₄O₅ | 535.4 |

### Example A-16: Preparation of Macrocyclic Compound 168

### tert-butyl (4-(3-cyano-4-methylphenoxy)butyl)carbamate (162)

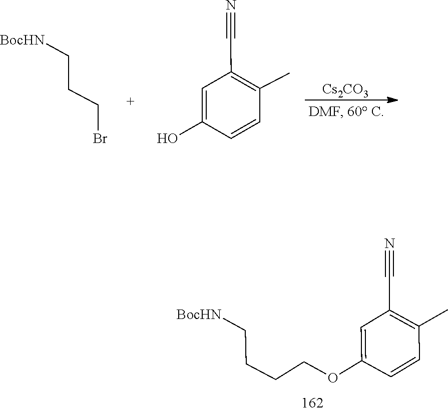

978 mg phenol, 2.5 g bromide and 6.46 g Cs₂CO₃ were stirred in 10 ml DMF at 60° C. until the phenol was consumed. The reaction mixture was diluted with DCM and washed with brine. Solvent was removed and the residue was purified by normal phase column chromatography (silica, cyclohexane/ethyl acetate gradient).

Formula: C₁₇H₂₄N₂O₃, exact mass: 304.2, found: 304 (GC), 205.2 [M+H-Boc]⁺

### tert-butyl (4-(3-(aminomethyl)-4-methylphenoxy)butyl)carbamate (163)

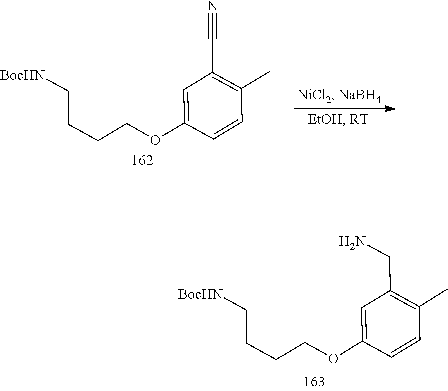 [SMILES: Cc1ccc(OCCCCNC(=O)OC(C)(C)C)cc1C#N ; Cc1ccc(OCCCCNC(=O)OC(C)(C)C)cc1CN]

To 1.55 g nitrile 162 an 660 mg NiCl₂ in ethanol 680 mg NaBH₄ were added in portions at room temperature. After complete reaction the mixture was filtered over a pad of Celite. Crude was purified by normal phase column chromatography (silica, DCM/methanol gradient).

Formula: C₁₇H₂₈N₂O₃, exact mass: 308.2, found: 309.2 [M+H]⁺

### (S)-tert-butyl-(4-(3-((2-(((benzyloxy)carbonyl)amino)-4-phenylbutanamido)methyl)-4-methylphenoxy)butyl)carbamate (164)

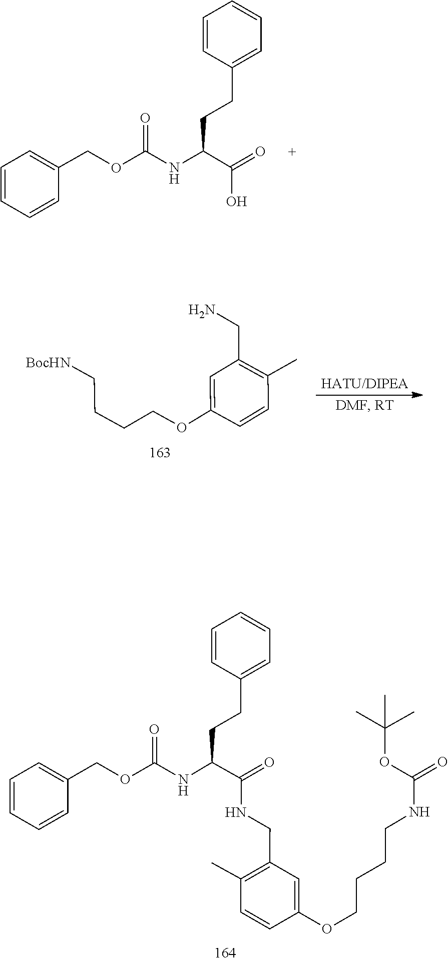 [SMILES: O=C(N[C@@H](CCc1ccccc1)C(=O)O)OCc1ccccc1 ; Cc1ccc(OCCCCNC(=O)OC(C)(C)C)cc1CN ; Cc1ccc(OCCCCNC(=O)OC(C)(C)C)cc1CNC(=O)[C@H](CCc1ccccc1)NC(=O)OCc1ccccc1]

The amide bond was formed according to general procedure B with 346 mg amine 163 and 421 mg Cbz-hPhe-OH. Purification was achieved by normal phase column chromatography (silica, cyclohexane/ethyl acetate gradient).

Formula: C₃₅H₄₅N₃O₆, exact mass: 603.3, found: 604.4 [M+H]⁺

### (S)-tert-butyl (4-(3-((2-amino-4-phenylbutanamido)methyl)-4-methylphenoxy)butyl) carbamate (165)

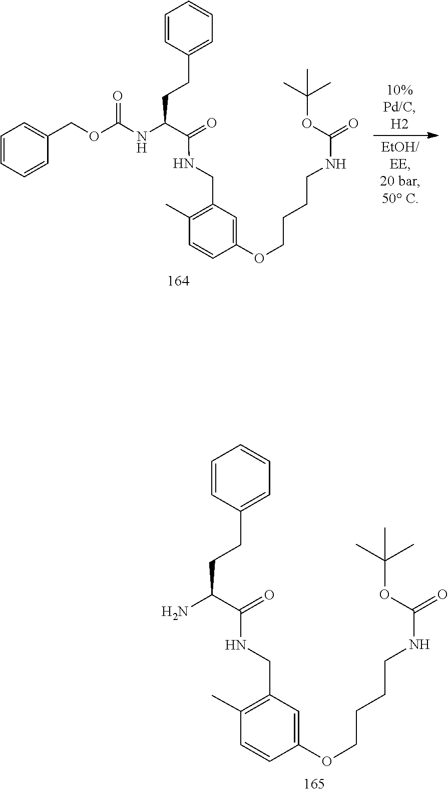 [SMILES: Cc1ccc(OCCCCNC(=O)OC(C)(C)C)cc1CNC(=O)[C@H](CCc1ccccc1)NC(=O)OCc1ccccc1 ; Cc1ccc(OCCCCNC(=O)OC(C)(C)C)cc1CNC(=O)[C@@H](N)CCc1ccccc1]

Benzyl ester was cleaved according to general procedure C using 10% Pd/C catalyst cartridge, 20 bar at 50° C. in a solvent mixture of ethanol/ethyl acetate. Product was directly used after removing of volatiles.

Formula: C₂₇H₃₉N₃O₄, exact mass: 469.3, found: 470.4 [M+H]⁺

### (S)-tert-butyl 3-acetamido-4-(((S)-1-((5-(4-((tert-butoxycarbonyl)amino)butoxy)-2-methylbenzyl)amino)-1-oxo-4-phenylbutan-2-yl)amino)-4-oxobutanoate (166)

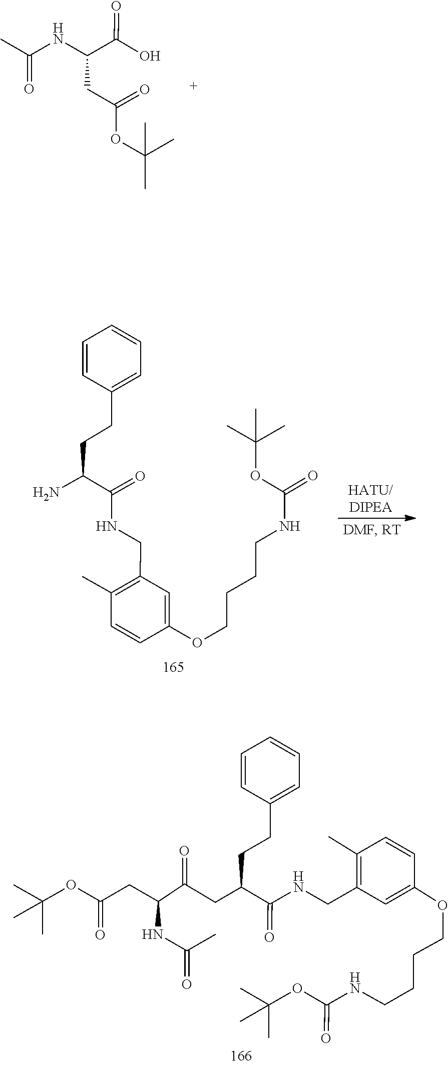 [SMILES: CC(=O)N[C@@H](CC(=O)OC(C)(C)C)C(=O)O ; Cc1ccc(OCCCCNC(=O)OC(C)(C)C)cc1CNC(=O)[C@@H](N)CCc1ccccc1 ; CC(=O)N[C@@H](CC(=O)OC(C)(C)C)C(=O)C[C@@H](CCc1ccccc1)C(=O)NCc1cc(OCCCCNC(=O)OC(C)(C)C)ccc1C]

The amide bond was formed according to general procedure B with 190 mg amine 165 and 112 mg Ac-Asp(OtBu)-OH. Purification was achieved by normal phase column chromatography (silica, cyclohexane/ethyl acetate gradient).

Formula: C₃₇H₅₄N₄O₈, exact mass: 682.4, found: 683.4 [M+H]⁺

### (S)-3-acetamido-4-(((S)-1-((5-(4-aminobutoxy)-2-methylbenzyl)amino)-1-oxo-4-phenylbutan-2-yl)amino)-4-oxobutanoic acid (167)

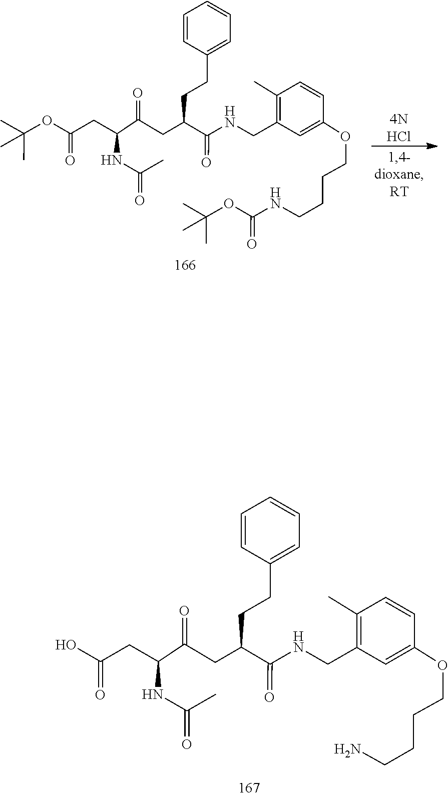 [SMILES: CC(=O)N[C@@H](CC(=O)OC(C)(C)C)C(=O)C[C@@H](CCc1ccccc1)C(=O)NCc1cc(OCCCCNC(=O)OC(C)(C)C)ccc1C ; CC(=O)N[C@@H](CC(=O)O)C(=O)C[C@@H](CCc1ccccc1)C(=O)NCc1cc(OCCCCN)ccc1C]

Protecting groups were cleaved from 276 mg compound 166 according to general procedure D stirring compounds in 6 ml 4 N HCl/1,4-dioxane at room temperature. After deprotection was complete solvent was removed and the crude dissolved in acetonitrile and concentrated twice. Product used without further purification.

Formula: C₂₈H₃₈N₄O₆, exact mass: 526.3, found: 527.3 [M+H]⁺

### N-((10S,13S)-1⁴-methyl-8,11,14-trioxo-13-phenethyl-2-oxa-7,12,15-triaza-1(1,3)-benzenacyclohexadecaphane-10-yl)acetamide (168)

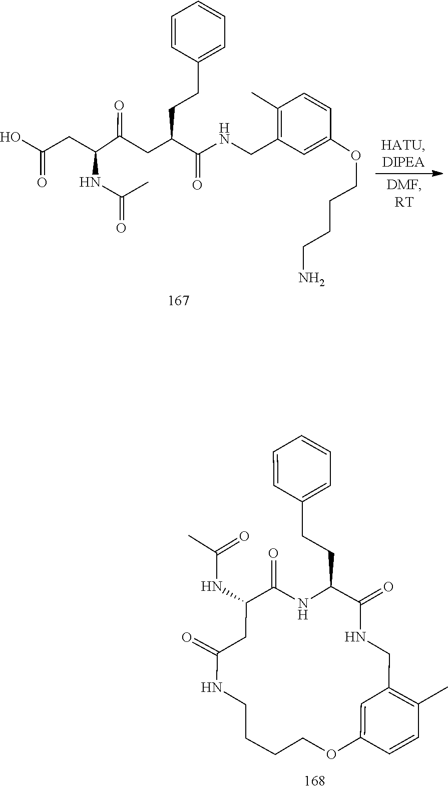 [SMILES: CC(=O)N[C@@H](CC(=O)O)C(=O)C[C@@H](CCc1ccccc1)C(=O)NCc1cc(OCCCCN)ccc1C ; CC(=O)N[C@H]1CC(=O)NCCCCOc2ccc(C)c(c2)CNC(=O)[C@H](CCc2ccccc2)NC1=O]

Macrocyclization was achieved according to general procedure A with 0.40 mmol amino acid 167. Reaction mixtures were diluted with some methanol and purified via HPLC.

Formula: C₂₈H₃₆N₄O₅, exact mass: 508.3, found: 509.3 [M+H]⁺

### Example A-17: Preparation of Macrocyclic Compounds 171-173

### (S)-tert-butyl 4-acetamido-5-(((S)-1-((5-(4-((tert-butoxycarbonyl)amino)butoxy)-2-methylbenzyl)amino)-1-oxo-4-phenylbutan-2-yl)amino)-5-oxopentanoate (169)

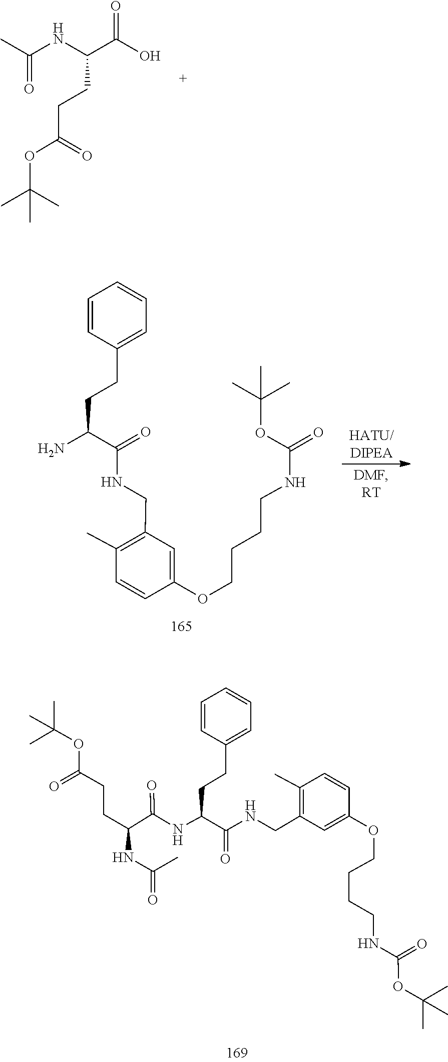 [SMILES: CC(=O)N[C@@H](CCC(=O)OC(C)(C)C)C(=O)O ; Cc1ccc(OCCCCNC(=O)OC(C)(C)C)cc1CNC(=O)[C@@H](N)CCc1ccccc1 ; CC(=O)N[C@@H](CCC(=O)OC(C)(C)C)C(=O)N[C@@H](CCc1ccccc1)C(=O)NCc1cc(OCCCCNC(=O)OC(C)(C)C)ccc1C]

The amide bond was formed according to general procedure B with 190 mg amine 165 and 119 mg Ac-Glut(OtBu)-OH. Purification was achieved by normal phase column chromatography (silica, cyclohexane/ethyl acetate gradient).

Formula: C₃₈H₅₆N₄O₈, exact mass: 696.4, found: 697.5 [M+H]⁺

### (S)-4-acetamido-5-(((S)-1-((5-(4-aminobutoxy)-2-methylbenzyl)amino)-1-oxo-4-phenylbutan-2-yl)amino-5-oxopentanoic acid (170)

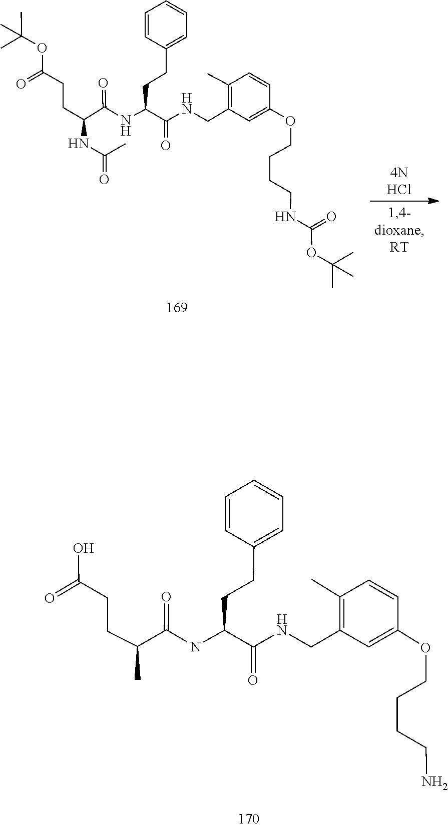 [SMILES: CC(=O)N[C@@H](CCC(=O)OC(C)(C)C)C(=O)N[C@@H](CCc1ccccc1)C(=O)NCc1cc(OCCCCNC(=O)OC(C)(C)C)ccc1C ; Cc1ccc(OCCCCN)cc1CNC(=O)[C@H](CCc1ccccc1)NC(=O)[C@@H](C)CCC(=O)O]

Protecting groups were cleaved from 282 mg compound 169 according to general procedure D stirring compounds in 6 ml 4 N HCl/1,4-dioxane at room temperature. After deprotection was complete solvent was removed and the crude dissolved in acetonitrile and concentrated twice. Product used without further purification.

Formula: C₂₉H₄ON₄O₆, exact mass: 540.3, found: 541.3 [M+H]⁺

### N-((11S,14S)-1⁴-methyl-8,12,15-trioxo-14-phenethyl-2-oxa-7,13,16-triaza-1(1,3)-benzenacycloheptadecaphane-11-yl)acetamide (171)

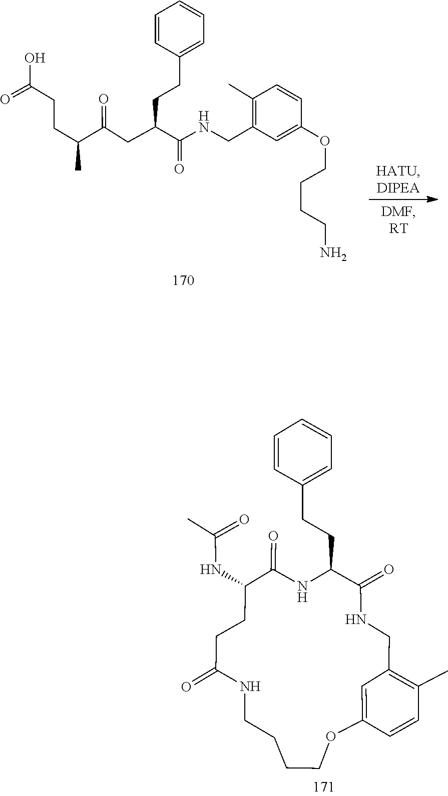 [SMILES: Cc1ccc(OCCCCN)cc1CNC(=O)[C@H](CCc1ccccc1)CC(=O)[C@@H](C)CCC(=O)O ; CC(=O)N[C@H]1CCC(=O)NCCCCOc2ccc(C)c(c2)CNC(=O)[C@H](CCc2ccccc2)NC1=O]

Macrocyclization was achieved according to general procedure A with 0.40 mmol amino acid 170. Reaction mixtures were diluted with some methanol and purified via HPLC.

Formula: C₂₉H₃₈N₄O₆, exact mass: 522.3, found: 523.3 [M+H]⁺

### 2-(4-aminophenyl)-N-((13R,9S,12S)-5⁴-methyl-8,11,14-trioxo-9-phenethyl-4-oxa-7,10-diaza-1(3,1)-piperidina-5(1,3)-benzenacyclotetradecaphane-12-yl)acetamide (172)

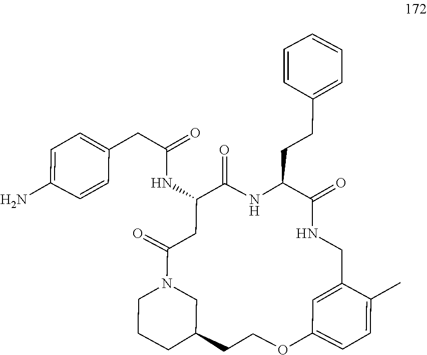 [SMILES: Cc1ccc2cc1CNC(=O)[C@H](CCc1ccccc1)NC(=O)[C@@H](NC(=O)Cc1ccc(N)cc1)CC(=O)N1CCC[C@H](CCO2)C1]

Compound 172 was prepared in analogy to compound 128 with the exception that (R)-tert-butyl 3-(2-hydroxyethyl)piperidine-1-carboxylate was used to synthesize a enantiomerically pure derivative of tert-butyl 3-(2-(3-cyano-4-methylphenoxy)ethyl)piperidine-1-carboxylate 3.

Formula: C₃₇H₄₅N₅O₅, exact mass: 639.3, found: 640.4 [M+H]⁺

### 2-(4-aminophenyl)-N-((13S,9S,12S)-5⁴-methyl-8,11,14-trioxo-9-phenethyl-4-oxa-7,10-diaza-1(3,1)-piperidina-5(1,3)-benzenacyclotetradecaphane-12-yl)acetamide (173)

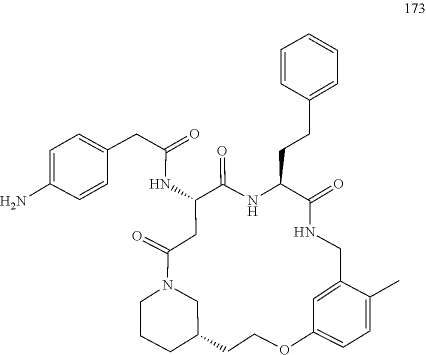 [SMILES: Cc1ccc2cc1CNC(=O)[C@H](CCc1ccccc1)NC(=O)[C@@H](NC(=O)Cc1ccc(N)cc1)CC(=O)N1CCC[C@@H](CCO2)C1]

Compound 172 was prepared in analogy to compound 128 with the exception that (S)-tert-butyl 3-(2-hydroxyethyl)piperidine-1-carboxylate was used to synthesize a enantiomerically pure derivative of tert-butyl 3-(2-(3-cyano-4-methylphenoxy)ethyl)piperidine-1-carboxylate 3.

Formula: C₃₇H₄₅N₅O₅, exact mass: 639.3, found: 640.4 [M+H]⁺

### Example A-18: Preparation of Macrocyclic Compounds 180, 181, 216, 217, and 294

### Amide Derivatizations of Compound 8 According to General Procedure E

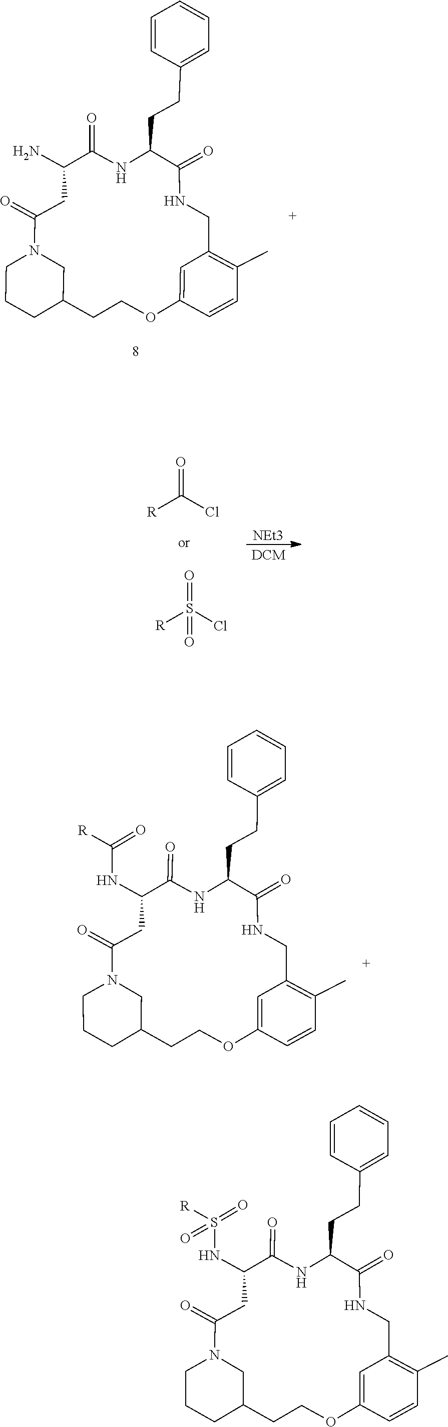

Compounds were synthesized according to general procedure E with e.g. 20 to 100 mg amine 8 and carboxylic acid chlorides or sulfonyl chlorides to give amides or sulfonamides as disclosed in table below. Purifications were achieved by HPLC or reversed phase flash chromatography.

TABLE A-13

| Cpd | | | Exact | [M + H]⁺ |
|---|---|---|---|---|
| no | structure | name | mass | found |
| 180 | 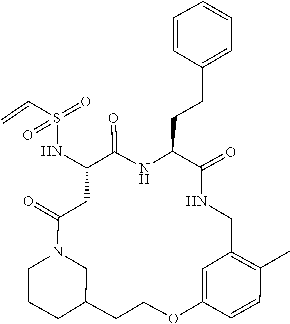 [SMILES: C=CS(=O)(=O)N[C@H]1CC(=O)N2CCCC(CCOc3ccc(C)c(c3)CNC(=O)[C@H](CCc3ccccc3)NC1=O)C2] | N-((9S,12S)-5⁴-methyl-8,11,14- trioxo-9-phenethyl-4-oxa-7,10-diaza- 1(3,1)-piperidina-5(1,3)- benzenacyclotetradecaphane-12- yl)ethenesulfonamide | 596.3 C₃₁H₄₀ N₄O₆S | 597.4 |
| 181 | 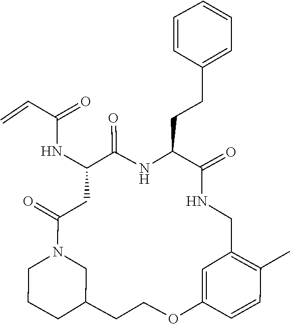 [SMILES: C=CC(=O)N[C@H]1CC(=O)N2CCCC(CCOc3ccc(C)c(c3)CNC(=O)[C@H](CCc3ccccc3)NC1=O)C2] | N-((9S,12S)-5⁴-methyl-8,11,14- trioxo-9-phenethyl-4-oxa-7,10-diaza- 1(3,1)-piperidina-5(1,3)- benzenacyclotetradecaphane-12- yl)acrylamide | 560.3 C₃₂H₄₀ N₄O₅ | 561.4 |
| 216 | 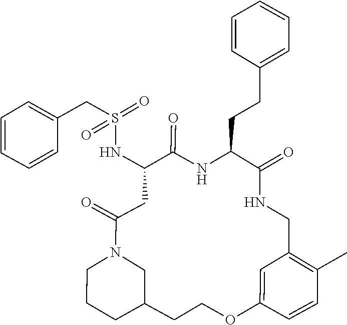 [SMILES: Cc1ccc2cc1CNC(=O)[C@H](CCc1ccccc1)NC(=O)[C@@H](NS(=O)(=O)Cc1ccccc1)CC(=O)N1CCCC(CCO2)C1] | N-((9S,12S)-5⁴-methyl-8,11,14- trioxo-9-phenethyl-4-oxa-7,10-diaza- 1(3,1)-piperidina-5(1,3)- benzenacyclotetradecaphane-12-yl)- 1-phenylmethanesulfonamide | 660.3 C₃₆H₄₄ N₄O₆S | 661.7 |
| 217 | 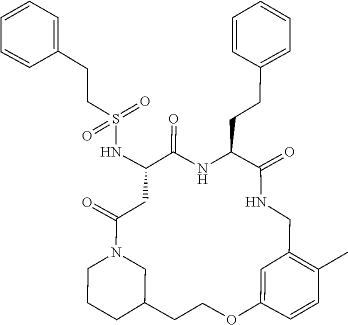 [SMILES: Cc1ccc2cc1CNC(=O)[C@H](CCc1ccccc1)NC(=O)[C@@H](NS(=O)(=O)CCc1ccccc1)CC(=O)N1CCCC(CCO2)C1] | N-((9S,12S)-5⁴-methyl-8,11,14- trioxo-9-phenethyl-4-oxa-7,10-diaza- 1(3,1)-piperidina-5(1,3)- benzenacyclotetradecaphane-12-yl)- 2-phenylethane-1-sulfonamide | 674.3 C₃₇H₄₆ N₄O₆S | 675.7 |
| 294 | 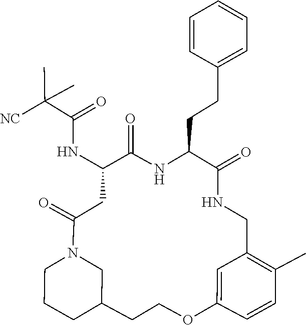 [SMILES: Cc1ccc2cc1CNC(=O)[C@H](CCc1ccccc1)NC(=O)[C@@H](NC(=O)C(C)(C)C#N)CC(=O)N1CCCC(CCO2)C1] | 2-cyano-2-methyl-N-((9S,12S)-54- methyl-8,11,14-trioxo-9-phenethyl-4- oxa-7,10-diaza-1(3,1)-piperidina- 5(1,3)-benzenacyclotetradecaphane- 12-yl)propanamide | 601.3 C₃₄H₄₃ N₅O₅ | 602.5 |

### Example A-19: Preparation of Macrocyclic Compound 186

### tert-butyl 2-(2-(3-(((S)-2-((S)-2-acetamido-6-(tert-butoxy)-6-oxohexanamido)-4-phenylbutanamido)methyl)-4-methylphenoxy)ethyl)morpholine-4-carboxylate (184)

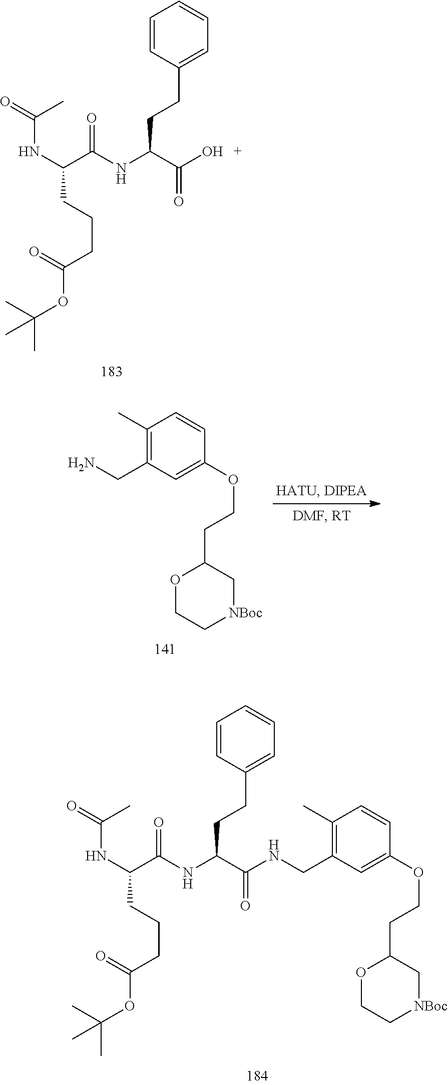

The amide bond was formed according to general procedure B with 140 mg amine 141 and 268 mg acid 183. Purification was achieved by normal phase column chromatography (silica, cyclohexane/ethyl acetate gradient).

Formula: C₄₁H₆₀N₄O₉, exact mass: 752.4, found: 753.7 [M+H]⁺

### (5S)-5-acetamido-6-(((2S)-1-((2-methyl-5-(2-(morpholin-2-yl)ethoxy)benzyl)amino)-1-oxo-4-phenylbutan-2-yl)amino)-6-oxohexanoic acid (185)

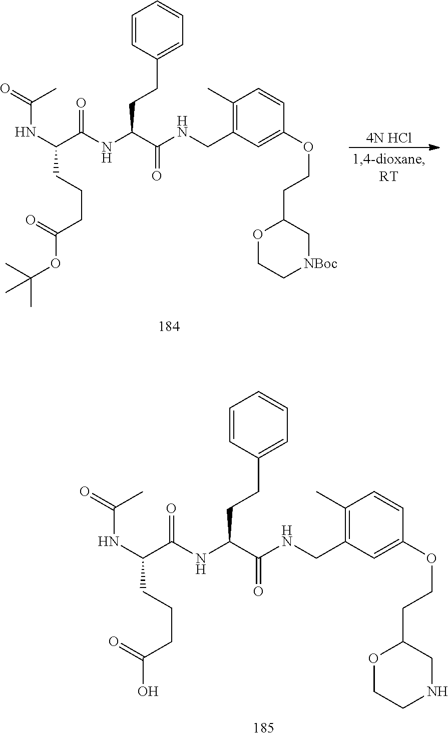

Protecting groups were cleaved from 99 mg compound 184 according to general procedure D stirring compounds in 10 ml 4 N HCl/1,4-dioxane at room temperature. After deprotection was complete solvent was removed and the crude dissolved in acetonitrile and concentrated twice. Product used without further purification

Formula: C₃₂H₄₄N₄O₇, exact mass: 596.3, found: 597.3 [M+H]⁺

### N-((9S,12S)-5⁴-methyl-8,11,16-trioxo-9-phenethyl-4-oxa-7,10-diaza-1(2,4)-morpholina-5(1,3)-benzenacyclohexadecaphane-12-yl)acetamide (186)

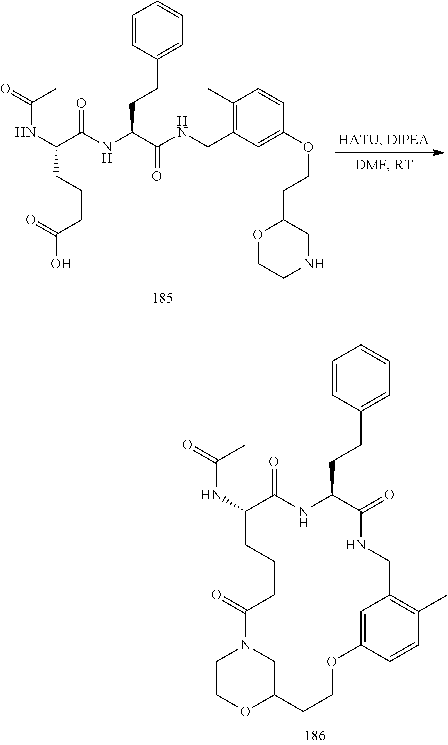 [SMILES: CC(=O)N[C@@H](CCCC(=O)O)C(=O)N[C@@H](CCc1ccccc1)C(=O)NCc1cc(OCCC2CNCCO2)ccc1C ; CC(=O)N[C@H]1CCCC(=O)N2CCOC(CCOc3ccc(C)c(c3)CNC(=O)[C@H](CCc3ccccc3)NC1=O)C2]

Macrocyclization was achieved according to general procedure A with 0.166 mmol amino acid 185. Reaction mixtures were diluted with some methanol and purified via HPLC. Formula: C₃₂H₄₂N₄O₆, exact mass: 578.3, found: 579.6 [M+H]⁺

### Example A-20: Preparation of Macrocyclic Compound 191

### tert-butyl 6-(3-(((S)-2-((S)-2-acetamido-6-(tert-butoxy)-6-oxohexanamido)-4-phenylbutanamido)methyl)-4-methylphenoxy)-3-azabicyclo[3.2.0]heptane-3-carboxylate (189)

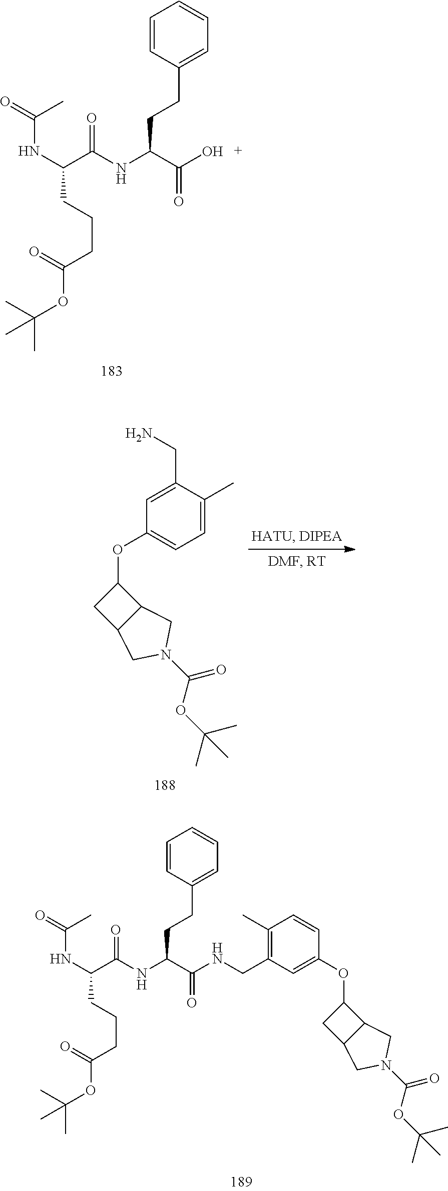

The amide bond was formed according to general procedure B with 134 mg amine 188 and 113 mg acid 183. Purification was achieved by normal phase column chromatography (silica, cyclohexane/ethyl acetate gradient).

Formula: C₄₁H₅₈N₄O₈, exact mass: 734.4, found: 735.7 [M+H]⁺

### (5S)-6-(((2S)-1-((5-(3-azabicyclo[3.2.0]heptan-6-yloxy)-2-methylbenzyl)amino)-1-oxo-4-phenylbutan-2-yl)amino)-5-acetamido-6-oxohexanoic acid (190)

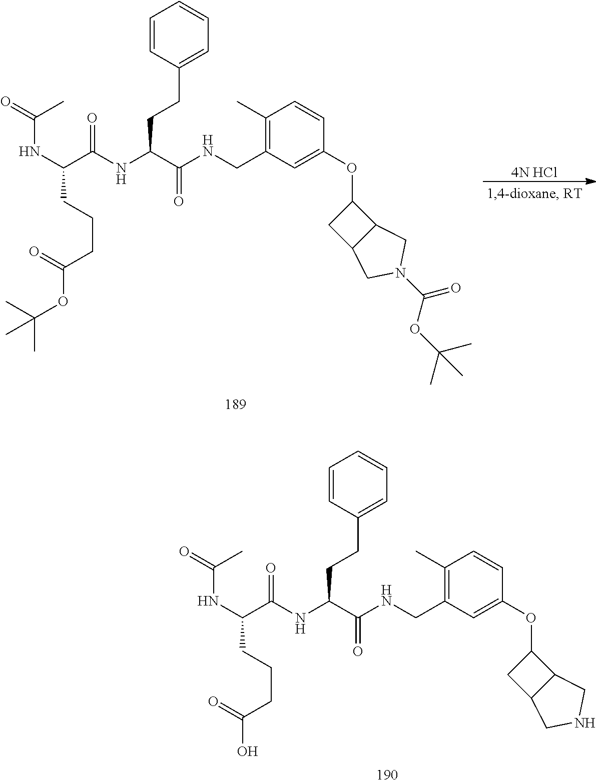 [SMILES: CC(=O)N[C@@H](CCCC(=O)OC(C)(C)C)C(=O)N[C@@H](CCc1ccccc1)C(=O)NCc1cc(OC2CC3CN(C(=O)OC(C)(C)C)CC32)ccc1C ; "4N HCl" ; CC(=O)N[C@@H](CCCC(=O)O)C(=O)N[C@@H](CCc1ccccc1)C(=O)NCc1cc(OC2CC3CNCC32)ccc1C]

Protecting groups were cleaved from 168 mg compound 189 according to general procedure D stirring compounds in 10 ml 4 N HCl/1,4-dioxane at room temperature. After deprotection was complete solvent was removed and the crude dissolved in acetonitrile and concentrated twice. Product used without further purification

Formula: C₃₂H₄₂N₄O₆, exact mass: 578.3, found: 579.3 [M+H]⁺

### N-((11R,15S,7S,10S)-3⁴-methyl-6,9,14-trioxo-7-phenethyl-2-oxa-13,5,8-triaza-1(7,3)-bicyclo[3.2.0]heptana-3(1,3)-benzenacyclotetradecaphane-10-yl)acetamide (191)

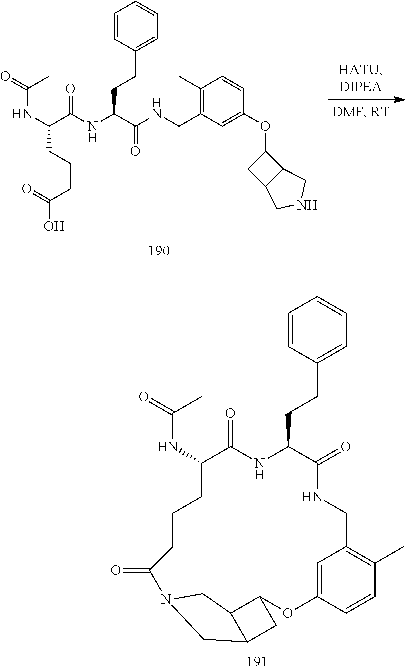

Macrocyclization was achieved according to general procedure A with 0.166 mmol amino acid 190. Reaction mixtures were diluted with some methanol and purified via HPLC.

Formula: C₃₂H₄ON₄O₅, exact mass: 560.3, found: 561.5 [M+H]⁺

### Example A-21: Preparation of Macrocyclic Compounds 347 and 353

### (S)-tert-butyl 6-(((benzyloxy)carbonyl)amino)-7-(((S)-1-((5-(3-((tert-butoxycarbonyl) amino)propoxy)-2-methylbenzyl)amino)-1-oxo-4-phenylbutan-2-yl)amino)-7-oxoheptanoate (446)

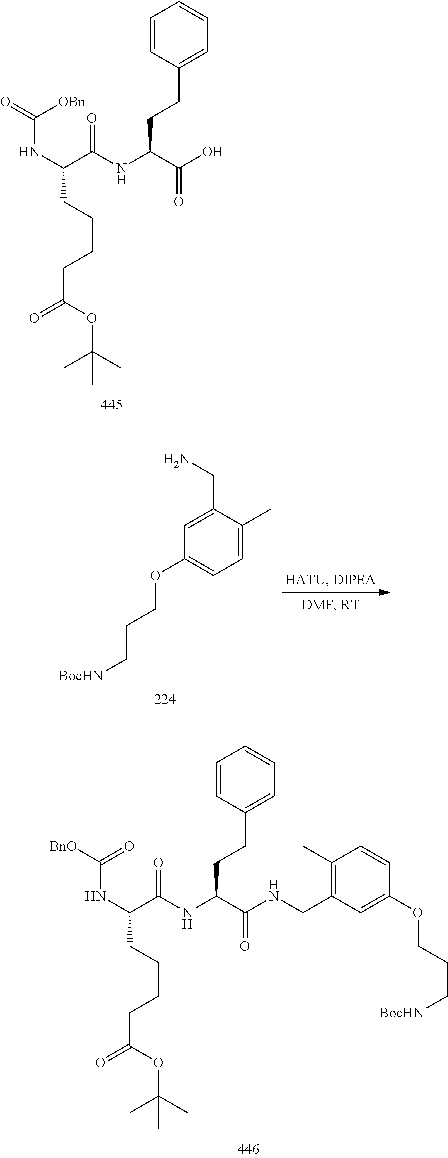

The amide bond was formed according to general procedure B with 187 mg amine 224 and 222 mg acid 445. Purification was achieved by normal phase column chromatography (silica, cyclohexane/ethyl acetate gradient). Additional examples utilizing other amines than amine 4 are disclosed in the following table.

TABLE A-14

| Cpd | From | | | Exact | [M + H]⁺ |
|---|---|---|---|---|---|
| no | amine | structure | name | mass | found |
| 446 | 224 | 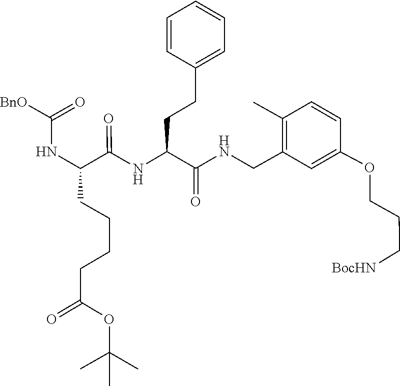 [SMILES: Cc1ccc(OCCCNC(=O)OC(C)(C)C)cc1CNC(=O)[C@H](CCc1ccccc1)NC(=O)[C@H](CCCCC(=O)OC(C)(C)C)NC(=O)OCc1ccccc1] | (S)-tert-butyl 6- (((benzyloxy)carbonyl)amino)- 7-(((S)-1-((5-(3-((tert- butoxycarbonyl)amino)propoxy)- 2-methylbenzyl)amino)-1-oxo- 4-phenylbutan-2-yl)amino)- 7-oxoheptanoate | 802.5 C₄₅H₆₂ N₄O₉ | 703.4 |
| 447 | 163 | 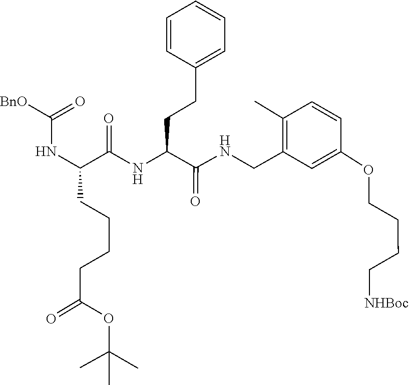 [SMILES: Cc1ccc(OCCCCNC(=O)OC(C)(C)C)cc1CNC(=O)[C@H](CCc1ccccc1)NC(=O)[C@H](CCCCC(=O)OC(C)(C)C)NC(=O)OCc1ccccc1] | (S)-tert-butyl 6- (((benzyloxy)carbonyl)amino)- 7-(((S)-1-((5-(4-((tert- butoxycarbonyl)amino)butoxy)- 2-methylbenzyl)amino)- 1-oxo-4-phenylbutan-2- yl)amino)-7-oxoheptanoate | 816.5 C₄₆H₆₄ N₄O₉ | 717.5 [M + H − Boc]⁺ |
| 464 | | 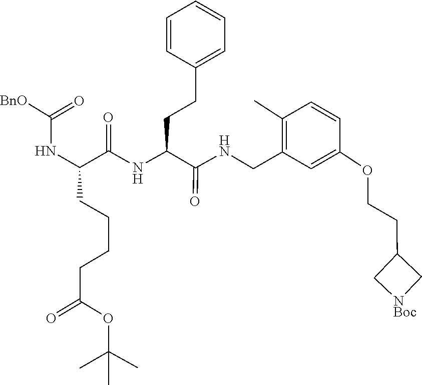 [SMILES: Cc1ccc(OCCC2CN(C(=O)OC(C)(C)C)C2)cc1CNC(=O)[C@H](CCc1ccccc1)NC(=O)[C@H](CCCCC(=O)OC(C)(C)C)NC(=O)OCc1ccccc1] | tert-butyl 3-(2-(3-((5S,8S)-5- (5-(tert-butoxy)-5- oxopentyl)-3,6,9-trioxo-8- phenethyl-1-phenyl-2-oxa- 4,7,10-triazaundecan-11- yl)-4-methylphenoxy)ethyl) azetidine-1-carboxylate | 828.5 C₄₇H₆₄ N₄O₉ | 829.7 |

### (S)-7-(((S)-1-((5-(3-aminopropoxy)-2-methylbenzyl)amino)-1-oxo-4-phenylbutan-2-yl)amino)-6-(((benzyloxy)carbonyl)amino)-7-oxoheptanoic acid (448)

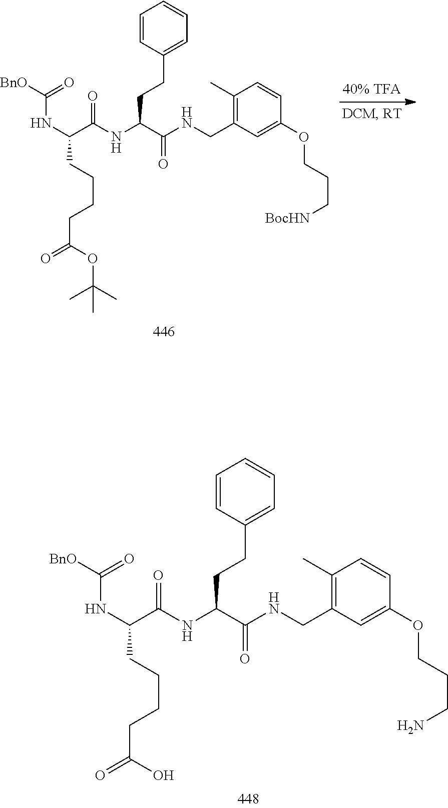 [SMILES: Cc1ccc(OCCCNC(=O)OC(C)(C)C)cc1CNC(=O)[C@H](CCc1ccccc1)NC(=O)[C@H](CCCCC(=O)OC(C)(C)C)NC(=O)OCc1ccccc1 ; Cc1ccc(OCCCN)cc1CNC(=O)[C@H](CCc1ccccc1)NC(=O)[C@H](CCCCC(=O)O)NC(=O)OCc1ccccc1]

Protecting groups were cleaved from 244 mg compound 446 according to general procedure D stirring compounds in 5 ml 40% TFA in DCM at room temperature. After deprotection was complete solvent was removed and the crude dissolved in acetonitrile and concentrated twice. Product used without further purification. Additional examples are disclosed in the following table.

TABLE A-15

| Cpd | From | | | Exact | [M + H]⁺ |
|---|---|---|---|---|---|
| no | cpd | structure | name | mass | found |
| 448 | 446 | 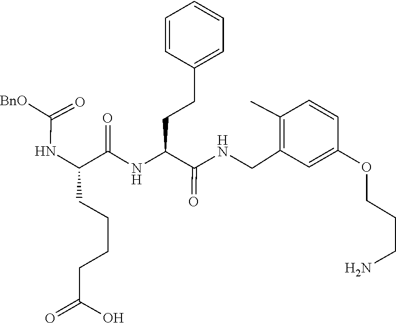 [SMILES: Cc1ccc(OCCCN)cc1CNC(=O)[C@H](CCc1ccccc1)NC(=O)[C@H](CCCCC(=O)O)NC(=O)OCc1ccccc1] | (S)-7-(((S)-1-((5-(3- aminopropoxy)-2- methylbenzyl)amino)-1- oxo-4-phenylbutan-2- yl)amino)-6- (((benzyloxy)carbonyl)amino)- 7-oxoheptanoic acid | 646.3 C₃₆H₄₆ N₄O₇ | 647.4 |
| 449 | 447 | 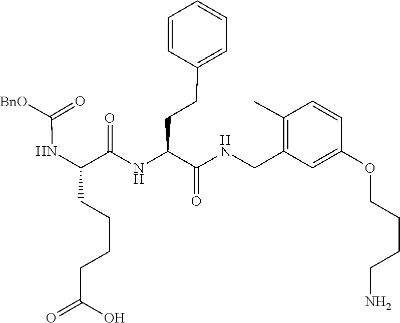 [SMILES: Cc1ccc(OCCCCN)cc1CNC(=O)[C@H](CCc1ccccc1)NC(=O)[C@H](CCCCC(=O)O)NC(=O)OCc1ccccc1] | (S)-7-(((S)-1-((5-(4- aminobutoxy)-2- methylbenzyl)amino)-1- oxo-4-phenylbutan-2- yl)amino)-6- (((benzyloxy)carbonyl)amino)- 7-oxoheptanoic acid | 660.4 C₃₇H₄₈ N₄O₇ | 661.4 |
| 465 | 464 | 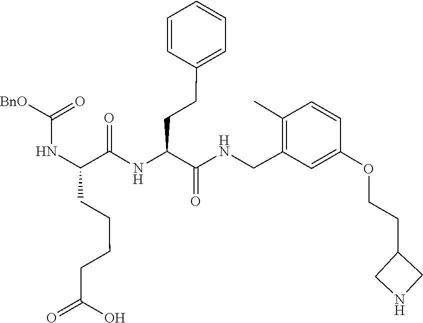 [SMILES: Cc1ccc(OCCC2CNC2)cc1CNC(=O)[C@H](CCc1ccccc1)NC(=O)[C@H](CCCCC(=O)O)NC(=O)OCc1ccccc1] | (S)-7-(((S)-1-((5-(2- (azetidin-3-yl)ethoxy)-2- methylbenzyl)amino)-1- oxo-4-phenylbutan-2- yl)amino)-6- (((benzyloxy)carbonyl)amino)- 7-oxoheptanoic acid | 672.4 C₃₈H₄₈ N₄O₇ | 673.4 |

### benzyl ((13S,16S)-1⁴-methyl-8,14,17-trioxo-16-phenethyl-2-oxa-7,15,18-triaza-1(1,3)-benzenacyclononadecaphane-13-yl)carbamate (347)

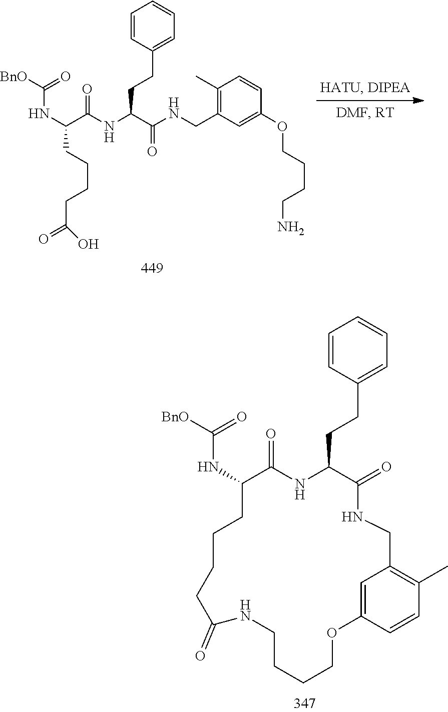 [SMILES: Cc1ccc(OCCCCN)cc1CNC(=O)[C@H](CCc1ccccc1)NC(=O)[C@H](CCCCC(=O)O)NC(=O)OCc1ccccc1 ; Cc1ccc2cc1CNC(=O)[C@H](CCc1ccccc1)NC(=O)[C@@H](NC(=O)OCc1ccccc1)CCCCC(=O)NCCCCO2]

Macrocyclization was achieved according to general procedure A with 0.3 mmol amino acid 449. Reaction mixtures were diluted with some methanol and purified via HPLC. Additional examples are disclosed in the following table.

TABLE A-16

| Cpd | From | | | Exact | [M + H]⁺ |
|---|---|---|---|---|---|
| no | cpd | structure | name | mass | found |
| 347 | 449 | 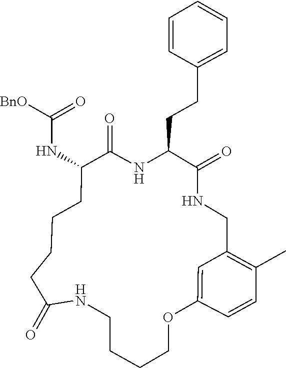 [SMILES: Cc1ccc2cc1CNC(=O)[C@H](CCc1ccccc1)NC(=O)[C@@H](NC(=O)OCc1ccccc1)CCCCC(=O)NCCCCO2] | benzyl ((13S,16S)-1⁴- methyl-8,14,17-trioxo-16- phenethyl-2-oxa-7,15,18- triaza-1(1,3)- benzenacyclononadecaphane- 13-yl)carbamate | 642.3 C₃₇H₄₆ N₄O₆ | 643.4 |
| 353 | 465 | 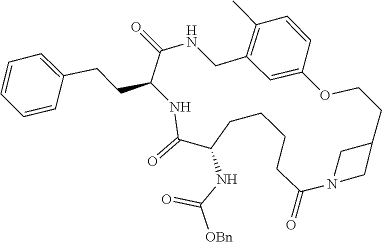 [SMILES: Cc1ccc2cc1CNC(=O)[C@H](CCc1ccccc1)NC(=O)[C@@H](NC(=O)OCc1ccccc1)CCCCC(=O)N1CC(CCO2)C1] | benzyl ((9S,12S)-5⁴-methyl- 8,11,17-trioxo-9-phenethyl- 4-oxa-7,10-diaza-1(3,1)- azetidina-5(1,3)- benzenacycloheptadecaphane- 12-yl)carbamate | 654.3 C₃₈H₄₆ N₄O₆ | 655.5 |

### Example A-22: Preparation of Macrocyclic Compounds 195, 283, 322, 351, 352, 354, and 405

### tert-butyl 3-(2-(3-((5S,8S)-5-(4-(tert-butoxy)-4-oxobutyl)-3,6,9-trioxo-8-phenethyl-1-phenyl-2-oxa-4,7,10-triazaundecan-11-yl)-4-methylphenoxy)ethyl)piperidine-1-carboxylate (193)

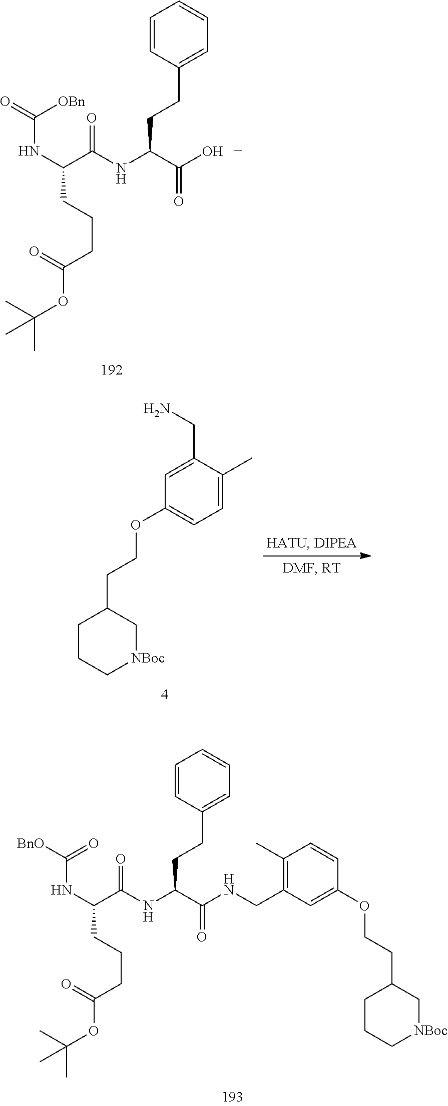

The amide bond was formed according to general procedure B with 615 mg amine 4 and 711 mg acid 192. Purification was achieved by normal phase column chromatography (silica, cyclohexane/ethyl acetate gradient). Additional examples utilizing other amines than amine 4 are disclosed in the following table.

TABLE A-17

| Cpd | From | | | Exact | [M + H]⁺ |
|---|---|---|---|---|---|
| no | amine | structure | name | mass | found |
| 193 | 4 | 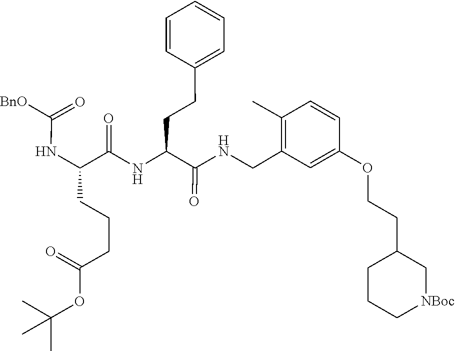 [SMILES: Cc1ccc(OCCC2CCCN(C(=O)OC(C)(C)C)C2)cc1CNC(=O)[C@H](CCc1ccccc1)NC(=O)[C@H](CCCC(=O)OC(C)(C)C)NC(=O)OCc1ccccc1] | tert-butyl 3-(2-(3- ((5S,8S)-5-(4-(tert- butoxy)-4-oxobutyl)- 3,6,9-trioxo-8-phenethyl- 1-phenyl-2-oxa-4,7,10- triazaundecan-11-yl)-4- methylphenoxy)ethyl) piperidine-1-carboxylate | 842.5 C₄₈H₆₆ N₄O₉ | 843.8 |
| 403 | 399 | 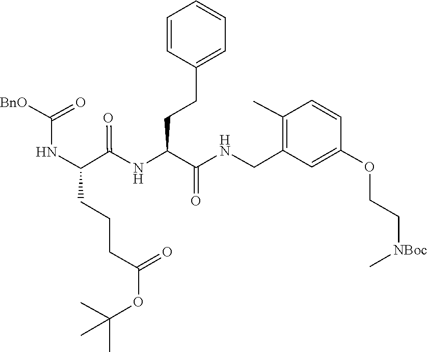 [SMILES: Cc1ccc(OCCN(C)C(=O)OC(C)(C)C)cc1CNC(=O)[C@H](CCc1ccccc1)NC(=O)[C@H](CCCC(=O)OC(C)(C)C)NC(=O)OCc1ccccc1] | (S)-tert-butyl 5- (((benzyloxy)carbonyl) amino)- 6-(((S)-1-((5-(2- ((tert- butoxycarbonyl)(methyl) amino)ethoxy)-2- methylbenzyl)amino)-1- oxo-4-phenylbutan-2- yl)amino)-6- oxohexanoate | 788.4 C₄₄H₆₀ N₄O₉ | 789.4 |
| 424 | 224 | 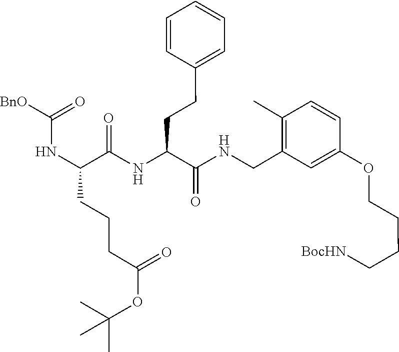 [SMILES: Cc1ccc(OCCCCNC(=O)OC(C)(C)C)cc1CNC(=O)[C@H](CCc1ccccc1)NC(=O)[C@H](CCCC(=O)OC(C)(C)C)NC(=O)OCc1ccccc1] | (S)-tert-butyl 5- (((benzyloxy)carbonyl) amino)- 6-(((S)-1-((5-(4- ((tert- butoxycarbonyl)amino) butoxy)- 2-methylbenzyl)amino)-1- oxo-4-phenylbutan-2- yl)amino)-6- oxohexanoate | 802.5 C₄₅H₆₂ N₄O₉ | 703.4 |
| 458 | 426 | 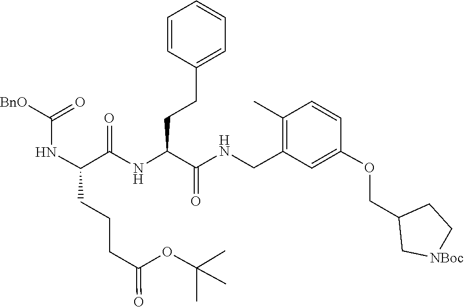 [SMILES: Cc1ccc(OCC2CCN(C(=O)OC(C)(C)C)C2)cc1CNC(=O)[C@H](CCc1ccccc1)NC(=O)[C@H](CCCC(=O)OC(C)(C)C)NC(=O)OCc1ccccc1] | tert-butyl 3-((3-((5S,8S)- 5-(4-(tert-butoxy)-4- oxobutyl)-3,6,9-trioxo-8- phenethyl-1-phenyl-2- oxa-4,7,10- triazaundecan-11-yl)-4- methylphenoxy)methyl) pyrrolidine-1-carboxylate | 814.5 C₄₆H₆₂ N₄O₉ | 815.7 |
| 459 | 455 | 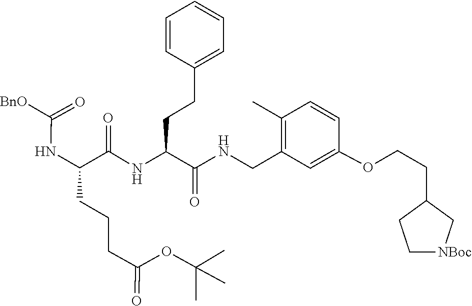 [SMILES: Cc1ccc(OCCC2CCN(C(=O)OC(C)(C)C)C2)cc1CNC(=O)[C@H](CCc1ccccc1)NC(=O)[C@H](CCCC(=O)OC(C)(C)C)NC(=O)OCc1ccccc1] | tert-butyl 3-(2-(3- ((5S,8S)-5-(4-(tert- butoxy)-4-oxobutyl)- 3,6,9-trioxo-8-phenethyl- 1-phenyl-2-oxa-4,7,10- triazaundecan-11-yl)-4- methylphenoxy)ethyl) pyrrolidine-1-carboxylate | 828.4 C₄₇H₆₄ N₄O₉ | 829.4 |
| 467 | 468 | 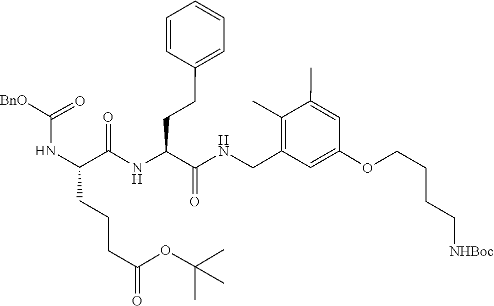 [SMILES: Cc1cc(OCCCCNC(=O)OC(C)(C)C)cc(CNC(=O)[C@H](CCc2ccccc2)NC(=O)[C@H](CCCC(=O)OC(C)(C)C)NC(=O)OCc2ccccc2)c1C] | (S)-tert-butyl 5- (((benzyloxy)carbonyl) amino)-6-(((S)-1-((5-(4- ((tert- butoxycarbonyl) amino)butoxy)- 2,3- dimethylbenzyl)amino)-1- oxo-4-phenylbutan-2- yl)amino)-6- oxohexanoate | 816.5 C₄₆H₆₄ N₄O₉ | 817.4 |
| 275 | 271 | 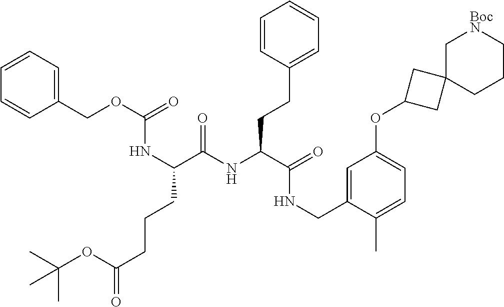 [SMILES: Cc1ccc(OC2CC3(CCCN(C(=O)OC(C)(C)C)C3)C2)cc1CNC(=O)[C@H](CCc1ccccc1)NC(=O)[C@H](CCCC(=O)OC(C)(C)C)NC(=O)OCc1ccccc1] | tert-butyl 2-(3-((5S,8S)-5- (4-(tert-butoxy)-4- oxobutyl)-3,6,9-trioxo-8- phenethyl-1-phenyl-2- oxa-4,7,10- triazaundecan-11-yl)-4- methylphenoxy)-6- azaspiro[3.5]nonane-6- carboxylate | 854.5 C₄₉H₆₆ N₄O₉ | 855.3 |

### (5S)-5-(((benzyloxy)carbonyl)amino)-6-(((2S)-1-((2-methyl-5-(2-(piperidin-3-yl)ethoxy)benzyl)amino)-1-oxo-4-phenylbutan-2-yl)amino)-6-oxohexanoic acid (194)

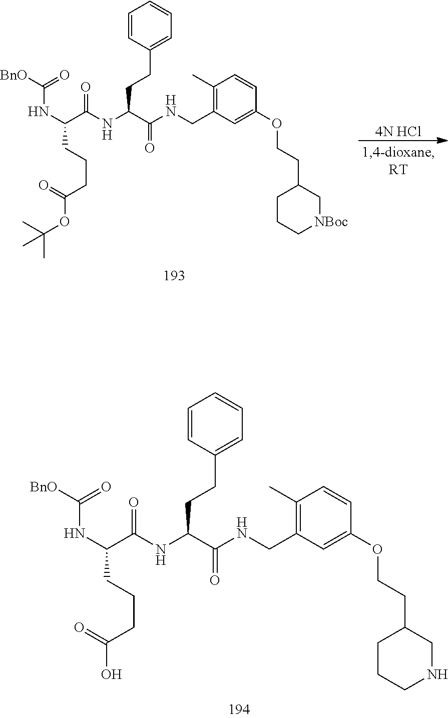 [SMILES: Cc1ccc(OCCC2CCCN(C(=O)OC(C)(C)C)C2)cc1CNC(=O)[C@H](CCc1ccccc1)NC(=O)[C@H](CCCC(=O)OC(C)(C)C)NC(=O)OCc1ccccc1 ; "4N HCl" ; Cc1ccc(OCCC2CCCNC2)cc1CNC(=O)[C@H](CCc1ccccc1)NC(=O)[C@H](CCCC(=O)O)NC(=O)OCc1ccccc1]

Protecting groups were cleaved from 900 mg compound 193 according to general procedure D stirring compounds in 10 ml 4 N HCl/1,4-dioxane at room temperature. After deprotection was complete solvent was removed and the crude dissolved in acetonitrile and concentrated twice. Product used without further purification. Additional examples are disclosed in the following table.

TABLE A-18

| Cpd | From | | | Exact | [M + H]⁺ |
|---|---|---|---|---|---|
| no | cpd | structure | name | mass | found |
| 194 | 193 | 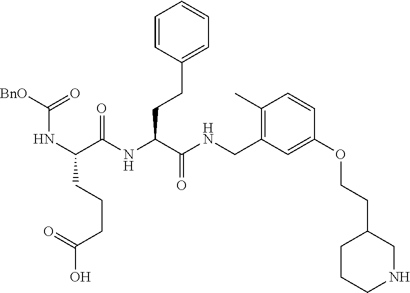 [SMILES: Cc1ccc(OCCC2CCCNC2)cc1CNC(=O)[C@H](CCc1ccccc1)NC(=O)[C@H](CCCC(=O)O)NC(=O)OCc1ccccc1] | (5S)-5- (((benzyloxy)carbonyl)amino)-6- (((2S)-1-((2-methyl-5-(2-(piperidin- 3-yl)ethoxy)benzyl)amino)-1-oxo-4- phenylbutan-2-yl)amino)-6- oxohexanoic acid | 686.4 C₃₉H₅₀ N₄O₇ | 687.4 |
| 404 | 403 | 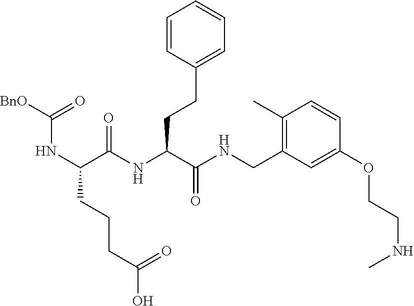 [SMILES: CNCCOc1ccc(C)c(CNC(=O)[C@H](CCc2ccccc2)NC(=O)[C@H](CCCC(=O)O)NC(=O)OCc2ccccc2)c1] | (S)-5-(((benzyloxy)carbonyl)amino)- 6-(((S)-1-((2-methyl-5-(2- (methylamino)ethoxy)benzyl)amino)- 1-oxo-4-phenylbutan-2-yl)amino)-6- oxohexanoic acid | 632.3 C₃₅H₄₄ N₄O₇ | — |
| 425 | 424 | 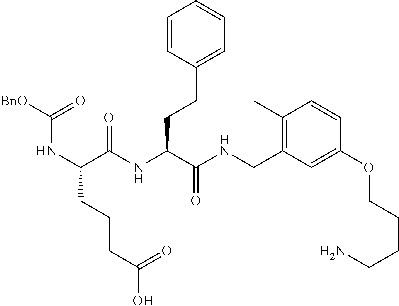 [SMILES: Cc1ccc(OCCCCN)cc1CNC(=O)[C@H](CCc1ccccc1)NC(=O)[C@H](CCCC(=O)O)NC(=O)OCc1ccccc1] | (S)-6-(((S)-1-((5-(4-aminobutoxy)-2- methylbenzyl)amino)-1-oxo-4- phenylbutan-2-yl)amino)-5- (((benzyloxy)carbonyl)amino)-6- oxohexanoic acid | 646.3 C₃₆H₄₆ N₄O₇ | 647.3 |
| 460 | 458 | 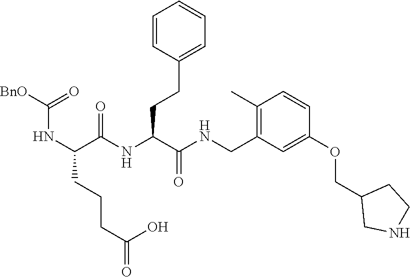 [SMILES: Cc1ccc(OCC2CCNC2)cc1CNC(=O)[C@H](CCc1ccccc1)NC(=O)[C@H](CCCC(=O)O)NC(=O)OCc1ccccc1] | (5S)-5- (((benzyloxy)carbonyl)amino)-6- (((2S)-1-((2-methyl-5-(pyrrolidin-3- ylmethoxy)benzyl)amino)-1-oxo-4- phenylbutan-2-yl)amino)-6- oxohexanoic acid | 658.3 C₃₇H₄₆ N₄O₇ | 659.6 |
| 461 | 459 | 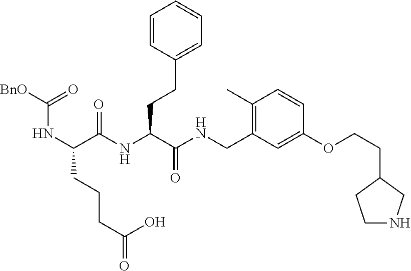 [SMILES: Cc1ccc(OCCC2CCNC2)cc1CNC(=O)[C@H](CCc1ccccc1)NC(=O)[C@H](CCCC(=O)O)NC(=O)OCc1ccccc1] | (5S)-5- (((benzyloxy)carbonyl)amino)-6- (((2S)-1-((2-methyl-5-(2-(pyrrolidin- 3-yl)ethoxy)benzyl)amino)-1-oxo-4- phenylbutan-2-yl)amino)-6- oxohexanoic acid | 672.4 C₃₈H₄₈ N₄O₇ | 673.4 |
| 469 | 467 | 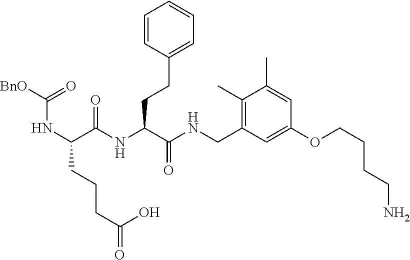 [SMILES: Cc1cc(OCCCCN)cc(CNC(=O)[C@H](CCc2ccccc2)NC(=O)[C@H](CCCC(=O)O)NC(=O)OCc2ccccc2)c1C] | (S)-6-(((S)-1-((5-(4-aminobutoxy)- 2,3-dimethylbenzyl)amino)-1-oxo-4- phenylbutan-2-yl)amino)-5- (((benzyloxy)carbonyl)amino)-6- oxohexanoic acid | 660.4 C₃₇H₄₈ N₄O₇ | 661.5 |
| 279 | 275 | 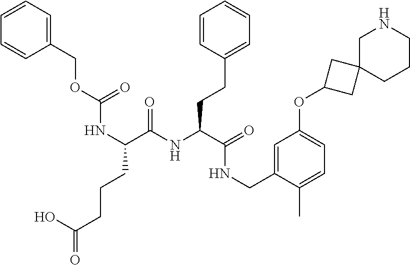 [SMILES: Cc1ccc(OC2CC3(CCCNC3)C2)cc1CNC(=O)[C@H](CCc1ccccc1)NC(=O)[C@H](CCCC(=O)O)NC(=O)OCc1ccccc1] | (S)-6-(((S)-1-((5-(6- azaspiro[3.5]nonan-2-yloxy)-2- methylbenzyl)amino)-1-oxo-4- phenylbutan-2-yl)amino)-5- (((benzyloxy)carbonyl)amino)-6- oxohexanoic acid | 698.4 C₄₀H₅₀ N₄O₇ | 699.4 |

### benzyl ((9S,12S)-5⁴-methyl-8,11,16-trioxo-9-phenethyl-4-oxa-7,10-diaza-1(3,1)-piperidina-5(1,3)-benzenacyclohexadecaphane-12-yl)carbamate (195)

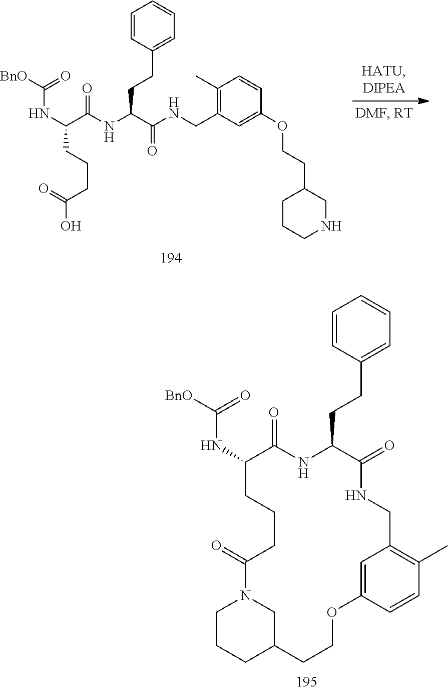 [SMILES: Cc1ccc(OCCC2CCCNC2)cc1CNC(=O)[C@H](CCc1ccccc1)NC(=O)[C@H](CCCC(=O)O)NC(=O)OCc1ccccc1 ; Cc1ccc2cc1CNC(=O)[C@H](CCc1ccccc1)NC(=O)[C@@H](NC(=O)OCc1ccccc1)CCCC(=O)N1CCCC(CCO2)C1]

Macrocyclization was achieved according to general procedure A with 1.07 mmol amino acid 194. Reaction mixtures were diluted with some methanol and purified via HPLC. Additional examples are disclosed in the following table.

TABLE A-19

| Cpd | From | | | Exact | [M + H]⁺ |
|---|---|---|---|---|---|
| no | cpd | structure | name | mass | found |
| 195 | 194 | 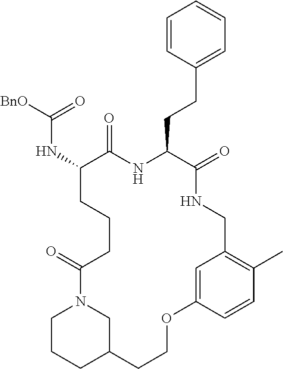 [SMILES: Cc1ccc2cc1CNC(=O)[C@H](CCc1ccccc1)NC(=O)[C@@H](NC(=O)OCc1ccccc1)CCCC(=O)N1CCCC(CCO2)C1] | benzyl ((9S,12S)-5⁴-methyl- 8,11,16-trioxo-9-phenethyl- 4-oxa-7, 10-diaza-1(3,1)- piperidina-5(1,3)- benzenacyclohexadecaphane- 12-yl)carbamate | 668.4 C₃₉H₄₈ N₄O₆ | 669.7 |
| 405 | 404 | 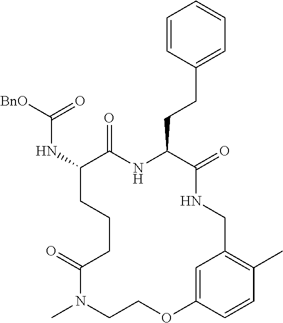 [SMILES: Cc1ccc2cc1CNC(=O)[C@H](CCc1ccccc1)NC(=O)[C@@H](NC(=O)OCc1ccccc1)CCCC(=O)N(C)CCO2] | benzyl ((10S,13S)-1⁴,5- dimethyl-6, 11,14-trioxo-13- phenethyl-2-oxa-5,12,15- triaza-1(1,3)- benzenacyclohexadecaphane- 10-yl)carbamate | 614.3 C₃₅H₄₂ N₄O₆ | 615.3 |
| 322 | 425 | 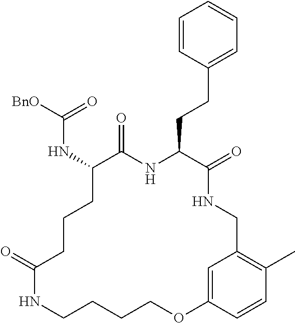 [SMILES: Cc1ccc2cc1CNC(=O)[C@H](CCc1ccccc1)NC(=O)[C@@H](NC(=O)OCc1ccccc1)CCCC(=O)NCCCCO2] | benzyl ((12S,15S)-1⁴- methyl-8,13, 16-trioxo-15- phenethyl-2-oxa-7,14,17- triaza-1(1,3)- benzenacyclooctadecaphane- 12-yl)carbamate | 628.3 C₃₆H₄₄ N₄O₆ | 629.4 |
| 351 | 460 | 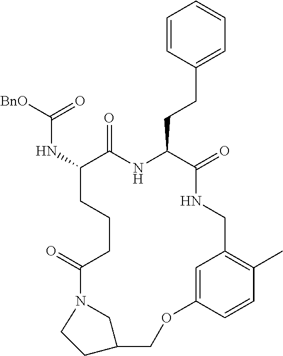 [SMILES: Cc1ccc2cc1CNC(=O)[C@H](CCc1ccccc1)NC(=O)[C@@H](NC(=O)OCc1ccccc1)CCCC(=O)N1CCC(CO2)C1] | benzyl ((8S, 11S)-4⁴-methyl- 7,10,15-trioxo-8-phenethyl- 3-oxa-6,9-diaza-1(3,1)- pyrrolidina-4(1,3)- benzenacyclopentadecaphane- 11-yl)carbamate | 640.3 C₃₇H₄₄ N₄O₆ | 641.5 |
| 352 | 461 | 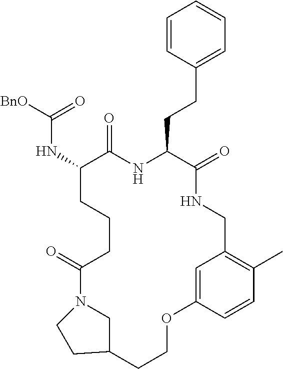 [SMILES: Cc1ccc2cc1CNC(=O)[C@H](CCc1ccccc1)NC(=O)[C@@H](NC(=O)OCc1ccccc1)CCCC(=O)N1CCC(CCO2)C1] | benzyl ((9S, 12S)-5⁴-methyl- 8,11,16-trioxo-9-phenethyl- 4-oxa-7,10-diaza-1(3,1)- pyrrolidina-5(1,3)- benzenacyclohexadecaphane- 12-yl)carbamate | 654.3 C₃₈H₄₆ N₄O₆ | 655.6 |
| 354 | 469 | 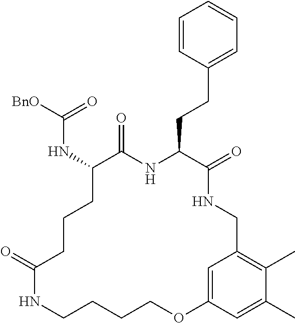 [SMILES: Cc1cc2cc(c1C)CNC(=O)[C@H](CCc1ccccc1)NC(=O)[C@@H](NC(=O)OCc1ccccc1)CCCC(=O)NCCCCO2] | benzyl ((12S,15S)-14,15- dimethyl-8,13,16-trioxo-15- phenethyl-2-oxa-7,14,17- triaza-1(1,3)- benzenacyclooctadecaphane- 12-yl)carbamate | 642.3 C₃₇H₄₆ N₄O₆ | 643.5 |
| 283 | 279 | 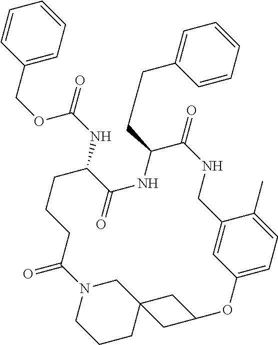 [SMILES: Cc1ccc2cc1CNC(=O)[C@H](CCc1ccccc1)NC(=O)[C@@H](NC(=O)OCc1ccccc1)CCCC(=O)N1CCCC3(CC(C3)O2)C1] | benzyl ((7S,10S)-34-methyl- 6,9,14-trioxo-7-phenethyl-2- oxa-5,8-diaza-15(3,1)- piperidina-3(1,3)-benzena- 1(1,3)- cyclobutanapentadecaphane- 10-yl)carbamate | 680.4 C₄₀H₄₈ N₄O₆ | 681.5 |

### Example A-23: Preparation of Macrocyclic Compounds 199, 301, 349, 350 and 402

### tert-butyl 3-(2-(3-((5S,8S)-5-(3-(tert-butoxy)-3-oxopropyl)-3,6,9-trioxo-8-phenethyl-1-phenyl-2-oxa-4,7,10-triazaundecan-11-yl)-4-methylphenoxy)ethyl)piperidine-1-carboxylate (197)

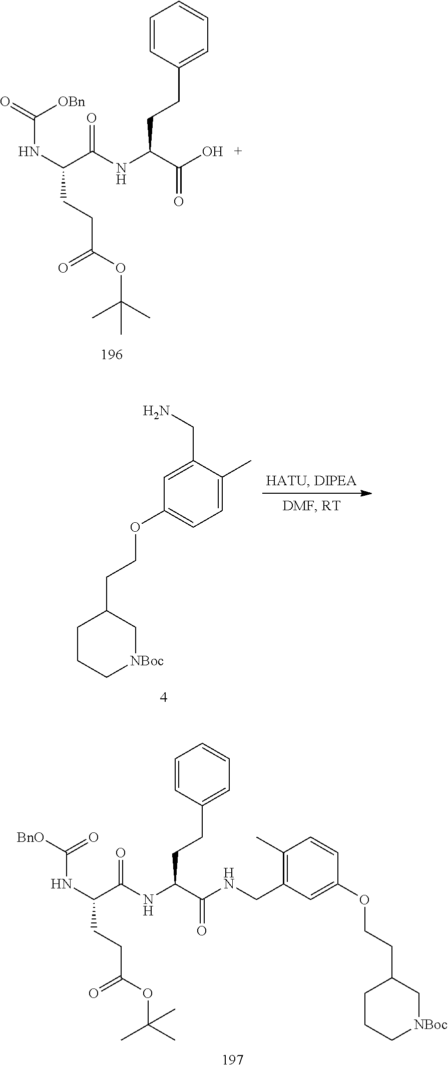

The amide bond was formed according to general procedure B with 615 mg amine 4 and 808 mg acid 196. Purification was achieved by normal phase column chromatography (silica, cyclohexane/ethyl acetate gradient). Additional examples utilizing other amines than amine 4 are disclosed in the following table.

TABLE A-20

| Cpd | From | | | Exact | [M + H]⁺ |
|---|---|---|---|---|---|
| no | amine | structure | name | mass | found |
| 197 | 4 | 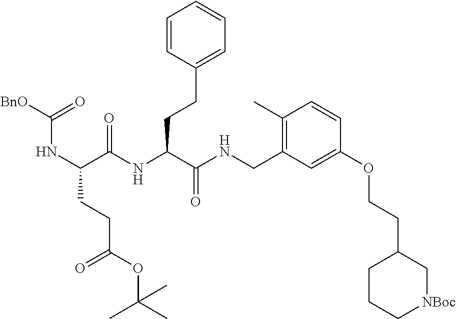 [SMILES: Cc1ccc(OCCC2CCCN(C(=O)OC(C)(C)C)C2)cc1CNC(=O)[C@H](CCc1ccccc1)NC(=O)[C@H](CCC(=O)OC(C)(C)C)NC(=O)OCc1ccccc1] | tert-butyl 3-(2-(3- ((5S,8S)-5-(3-(tert- butoxy)-3-oxopropyl)- 3,6,9-trioxo-8-phenethyl- 1-phenyl-2-oxa-4,7,10- triazaundecan-11-yl)-4- methylphenoxy)ethyl) piperidine-1-carboxylate | 828.5 C₄₇H₆₄ N₄O₉ | 830.6 |
| 385 | 271 | 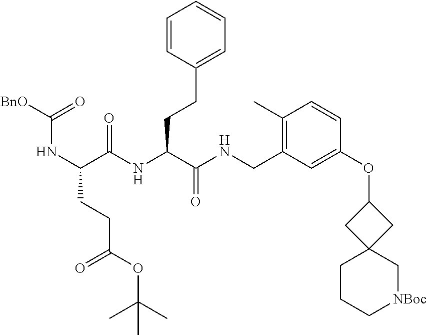 [SMILES: Cc1ccc(OC2CC3(CCCN(C(=O)OC(C)(C)C)C3)C2)cc1CNC(=O)[C@H](CCc1ccccc1)NC(=O)[C@H](CCC(=O)OC(C)(C)C)NC(=O)OCc1ccccc1] | tert-butyl 2-(3-((5S,8S)-5- (3-(tert-butoxy)-3- oxopropyl)-3,6,9-trioxo-8- phenethyl-1-phenyl-2- oxa-4,7,10- triazaundecan-11-yl)-4- methylphenoxy)-6- azaspiro[3.5]nonane-6- carboxylate | 840.5 C₄₈H₆₄ N₄O₉ | 841.6 |
| 400 | 399 | 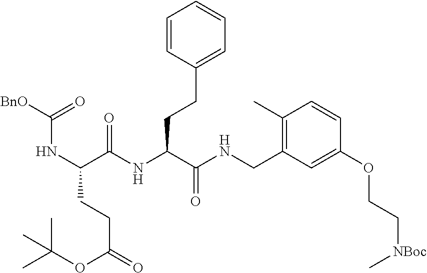 [SMILES: Cc1ccc(OCCN(C)C(=O)OC(C)(C)C)cc1CNC(=O)[C@H](CCc1ccccc1)NC(=O)[C@H](CCC(=O)OC(C)(C)C)NC(=O)OCc1ccccc1] | (S)-tert-butyl 4- (((benzyloxy)carbonyl)a mino)-5-(((S)-1-((5-(2- ((tert- butoxycarbonyl)(methyl) amino)ethoxy)-2- methylbenzyl)amino)-1- oxo-4-phenylbutan-2- yl)amino)-5- oxopentanoate | 774.4 C₄₃H₅₈ N₄O₉ | — |
| 451 | 426 | 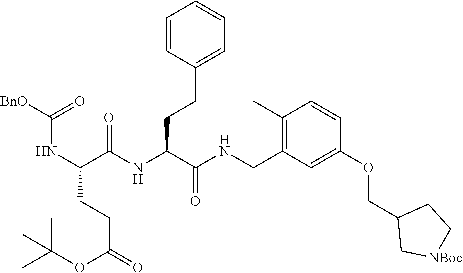 [SMILES: Cc1ccc(OCC2CCN(C(=O)OC(C)(C)C)C2)cc1CNC(=O)[C@H](CCc1ccccc1)NC(=O)[C@H](CCC(=O)OC(C)(C)C)NC(=O)OCc1ccccc1] | tert-butyl 3-((3-((5S,8S)- 5-(3-(tert-butoxy)-3- oxopropyl)-3,6,9-trioxo-8- phenethyl-1-phenyl-2- oxa-4,7,10- triazaundecan-11-yl)-4- methylphenoxy)methyl) pyrrolidine-1-carboxylate | 800.4 C₄₅H₆₀ N₄O₉ | 801.4 |
| 452 | 455 | 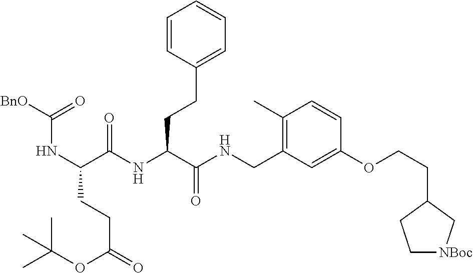 [SMILES: Cc1ccc(OCCC2CCN(C(=O)OC(C)(C)C)C2)cc1CNC(=O)[C@H](CCc1ccccc1)NC(=O)[C@H](CCC(=O)OC(C)(C)C)NC(=O)OCc1ccccc1] | tert-butyl 3-(2-(3- ((5S,8S)-5-(3-(tert- butoxy)-3-oxopropyl)- 3,6,9-trioxo-8-phenethyl- 1-phenyl-2-oxa-4,7,10- triazaundecan-11-yl)-4- methylphenoxy)ethyl) pyrrolidine-1-carboxylate | 814.5 C₄₆H₆₂ N₄O₉ | 815.4 |

### (4S)-4-(((benzyloxy)carbonyl)amino)-5-(((2S)-1-((2-methyl-5-(2-(piperidin-3-yl)ethoxy)benzyl)amino)-1-oxo-4-phenylbutan-2-yl)amino)-5-oxopentanoic acid (198)

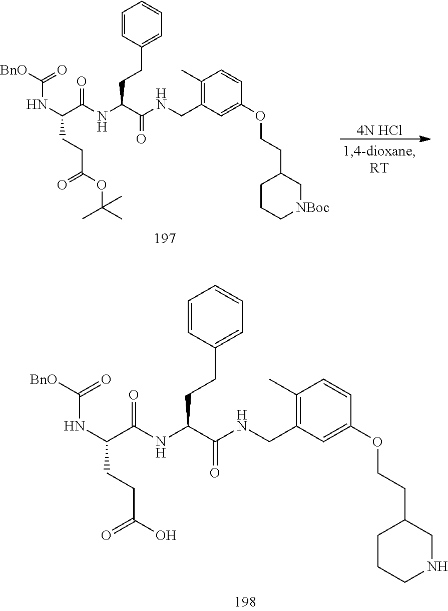 [SMILES: Cc1ccc(OCCC2CCCN(C(=O)OC(C)(C)C)C2)cc1CNC(=O)[C@H](CCc1ccccc1)NC(=O)[C@H](CCC(=O)OC(C)(C)C)NC(=O)OCc1ccccc1 ; "4N HCl" ; Cc1ccc(OCCC2CCCNC2)cc1CNC(=O)[C@H](CCc1ccccc1)NC(=O)[C@H](CCC(=O)O)NC(=O)OCc1ccccc1]

Protecting groups were cleaved from 850 mg compound 197 according to general procedure D stirring compounds in 10 ml 4 N HCl/1,4-dioxane at room temperature. After deprotection was complete solvent was removed and the crude dissolved in acetonitrile and concentrated twice. Product used without further purification. Additional examples are disclosed in the following table.

TABLE A-21

| Cpd | From | | | Exact | [M + H]⁺ |
|---|---|---|---|---|---|
| no | cpd | structure | name | mass | found |
| 198 | 197 | 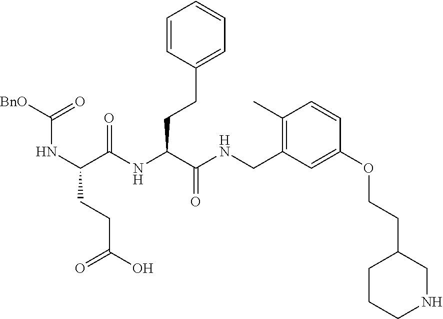 [SMILES: Cc1ccc(OCCC2CCCNC2)cc1CNC(=O)[C@H](CCc1ccccc1)NC(=O)[C@H](CCC(=O)O)NC(=O)OCc1ccccc1] | (4S)-4- (((benzyloxy)carbonyl)amino)- 5-(((2S)-1-((2-methyl-5- (2-(piperidin-3- yl)ethoxy)benzyl)amino)-1- oxo-4-phenylbutan-2- yl)amino)-5-oxopentanoic acid | 672.4 C₃₈H₄₈N₄O₇ | 673.7 |
| 386 | 385 | 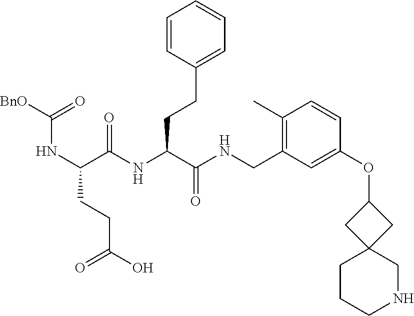 [SMILES: Cc1ccc(OC2CC3(CCCNC3)C2)cc1CNC(=O)[C@H](CCc1ccccc1)NC(=O)[C@H](CCC(=O)O)NC(=O)OCc1ccccc1] | (S)-5-(((S)-1-((5-(6- azaspiro[3.5]nonan-2- yloxy)-2- methylbenzyl)amino)-1-oxo- 4-phenylbutan-2-yl)amino)- 4- (((benzyloxy)carbonyl)amino)- 5-oxopentanoic acid | 684.4 C₃₉H₄₈N₄O₇ | 685.4 |
| 401 | 400 | 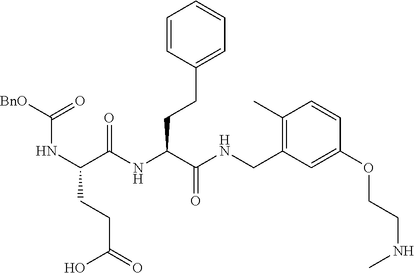 [SMILES: CNCCOc1ccc(C)c(CNC(=O)[C@H](CCc2ccccc2)NC(=O)[C@H](CCC(=O)O)NC(=O)OCc2ccccc2)c1] | (S)-4- (((benzyloxy)carbonyl)amino)- 5-(((S)-1-((2-methyl-5-(2- (methylamino)ethoxy)benzyl) amino)-1-oxo-4- phenylbutan-2-yl)amino)-5- oxopentanoic acid | 618.3 C₃₄H₄₂N₄O₇ | — |
| 456 | 451 | 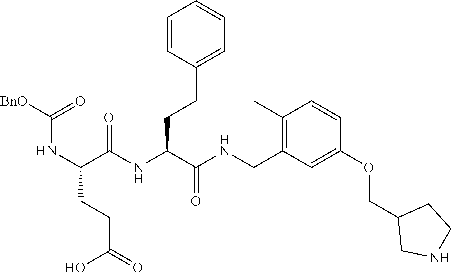 [SMILES: Cc1ccc(OCC2CCNC2)cc1CNC(=O)[C@H](CCc1ccccc1)NC(=O)[C@H](CCC(=O)O)NC(=O)OCc1ccccc1] | (4S)-4- (((benzyloxy)carbonyl)amino)- 5-(((2S)-1-((2-methyl-5- (pyrrolidin-3- ylmethoxy)benzyl)amino)-1- oxo-4-phenylbutan-2- yl)amino)-5-oxopentanoic acid | 644.3 C₃₆H₄₄N₄O₇ | 645.5 |
| 457 | 452 | 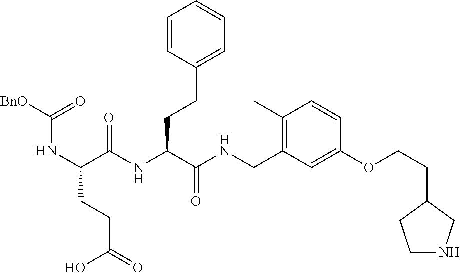 [SMILES: Cc1ccc(OCCC2CCNC2)cc1CNC(=O)[C@H](CCc1ccccc1)NC(=O)[C@H](CCC(=O)O)NC(=O)OCc1ccccc1] | (4S)-4- (((benzyloxy)carbonyl)amino)- 5-(((2S)-1-((2-methyl-5- (2-(pyrrolidin-3- yl)ethoxy)benzyl)amino)-1- oxo-4-phenylbutan-2- yl)amino)-5-oxopentanoic acid | 658.3 C₃₇H₄₆N₄O₇ | 629.5 |

### benzyl ((9S,12S)-5⁴-methyl-8,11,15-trioxo-9-phenethyl-4-oxa-7,10-diaza-1(3,1)-piperidina-5(1,3)-benzenacyclopentadecaphane-12-yl)carbamate (199)

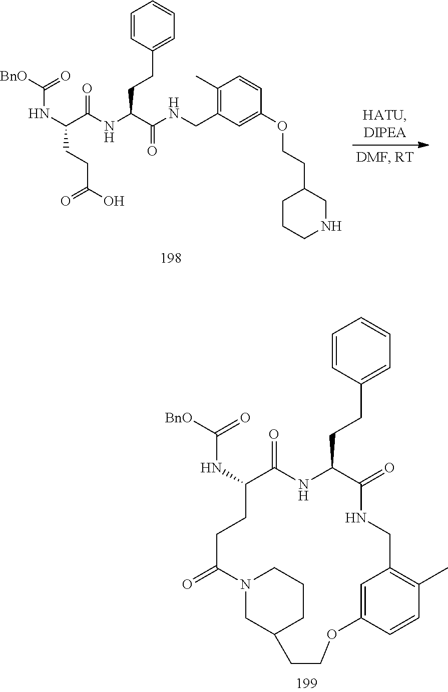 [SMILES: Cc1ccc(OCCC2CCCNC2)cc1CNC(=O)[C@H](CCc1ccccc1)NC(=O)[C@H](CCC(=O)O)NC(=O)OCc1ccccc1 ; Cc1ccc2cc1CNC(=O)[C@H](CCc1ccccc1)NC(=O)[C@@H](NC(=O)OCc1ccccc1)CCC(=O)N1CCCC(CCO2)C1]

Macrocyclization was achieved according to general procedure A with 1.03 mmol amino acid 198. Reaction mixtures were diluted with some methanol and purified via HPLC. Additional examples are disclosed in the following table.

TABLE A-22

| Cpd | From | | | Exact | [M + H]⁺ |
|---|---|---|---|---|---|
| no | cpd | structure | name | mass | found |
| 199 | 198 | 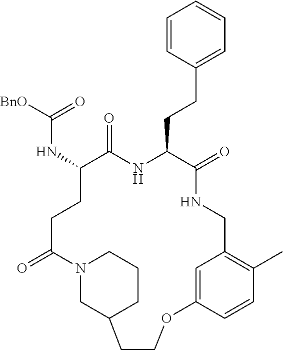 [SMILES: Cc1ccc2cc1CNC(=O)[C@H](CCc1ccccc1)NC(=O)[C@@H](NC(=O)OCc1ccccc1)CCC(=O)N1CCCC(CCO2)C1] | benzyl ((9S,12S)-5⁴-methyl- 8,11,15-trioxo-9-phenethyl-4- oxa-7,10-diaza-1(3,1)- piperidina-5(1,3)- benzenacyclopentadecaphane- 12-yl)carbamate | 654.3 C₃₈H₄₆N₄O₆ | 655.6 |
| 301 | 386 | 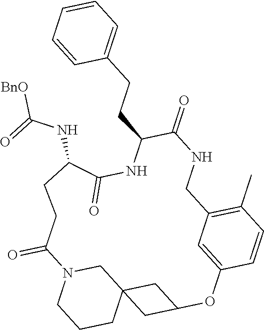 [SMILES: Cc1ccc2cc1CNC(=O)[C@H](CCc1ccccc1)NC(=O)[C@@H](NC(=O)OCc1ccccc1)CCC(=O)N1CCCC3(CC(C3)O2)C1] | benzyl ((7S,10S)-3⁴-methyl- 6,9,13-trioxo-7-phenethyl-2-oxa- 5,8-diaza-14(3,1)-piperidina- 3(1,3)-benzena-1(1,3)- cyclobutanatetradecaphane-10- yl)carbamate | 666.3 C₃₉H₄₆N₄O₆ | 667.4 |
| 402 | 401 | 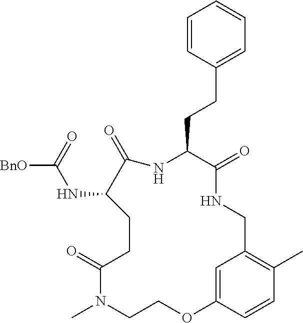 [SMILES: Cc1ccc2cc1CNC(=O)[C@H](CCc1ccccc1)NC(=O)[C@@H](NC(=O)OCc1ccccc1)CCC(=O)N(C)CCO2] | benzyl ((9S,12S)-1⁴,5-dimethyl- 6,10,13-trioxo-12-phenethyl-2- oxa-5,11,14-triaza-1(1,3)- benzenacyclopentadecaphane- 9-yl)carbamate | 600.3 C₃₄H₄₀N₄O₆ | 601.3 |
| 349 | 456 | 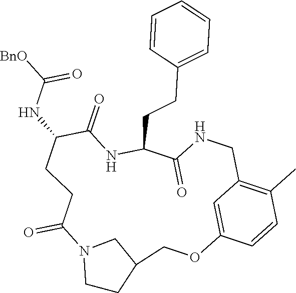 [SMILES: Cc1ccc2cc1CNC(=O)[C@H](CCc1ccccc1)NC(=O)[C@@H](NC(=O)OCc1ccccc1)CCC(=O)N1CCC(CO2)C1] | benzyl ((8S,11S)-4⁴-methyl- 7,10,14-trioxo-8-phenethyl-3- oxa-6,9-diaza-1(3,1)-pyrrolidina- 4(1,3)- benzenacyclotetradecaphane- 11-yl)carbamate | 626.3 C₃₆H₄₂N₄O₆ | 627.4 |
| 350 | 457 | 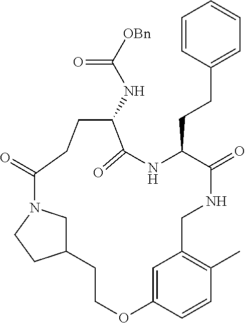 [SMILES: Cc1ccc2cc1CNC(=O)[C@H](CCc1ccccc1)NC(=O)[C@@H](NC(=O)OCc1ccccc1)CCC(=O)N1CCC(CCO2)C1] | benzyl ((9S,12S)-5⁴-methyl- 8,11,15-trioxo-9-phenethyl-4- oxa-7,10-diaza-1(3,1)- pyrrolidina-5(1,3)- benzenacyclopentadecaphane- 12-yl)carbamate | 640.3 C₃₇H₄₄N₄O₆ | 640.5 |

### Example A-24: Preparation of Macrocyclic Compounds 300, 312, 313, 314, 315, 325, and 337

### tert-butyl 2-(3-((5S,8S)-5-(2-(tert-butoxy)-2-oxoethyl)-3,6,9-trioxo-8-phenethyl-1-phenyl-2-oxa-4,7,10-triazaundecan-11-yl)-4-methylphenoxy)-6-azaspiro[3.5]nonane-6-carboxylate (383)

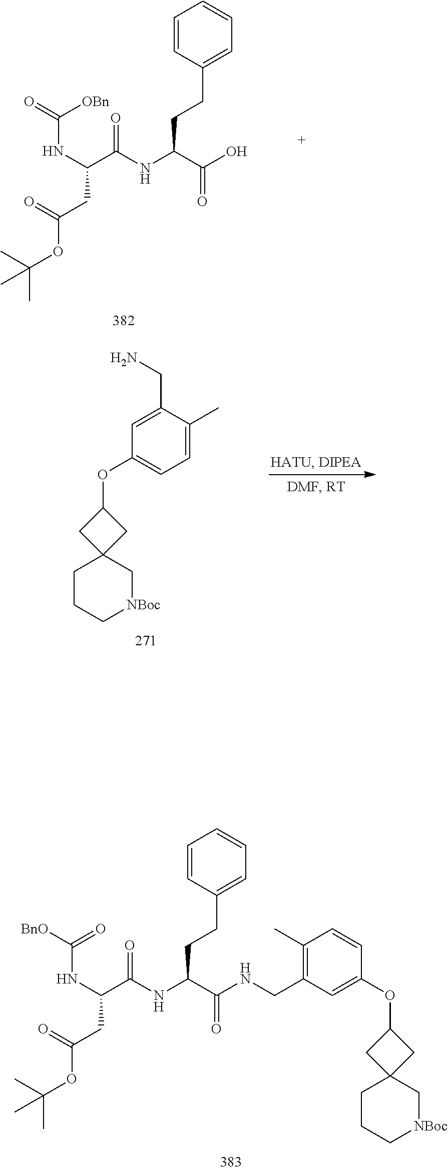 [SMILES: CC(C)(C)OC(=O)C[C@H](NC(=O)OCc1ccccc1)C(=O)N[C@@H](CCc1ccccc1)C(=O)O ; Cc1ccc(OC2CC3(CCCN(C(=O)OC(C)(C)C)C3)C2)cc1CN ; Cc1ccc(OC2CC3(CCCN(C(=O)OC(C)(C)C)C3)C2)cc1CNC(=O)[C@H](CCc1ccccc1)NC(=O)[C@H](CC(=O)OC(C)(C)C)NC(=O)OCc1ccccc1]

The amide bond was formed according to general procedure B with 100 mg amine 271 and 336 mg acid 382. Purification was achieved by normal phase column chromatography (silica, cyclohexane/ethyl acetate gradient). Additional examples utilizing other amines than amine 271 are disclosed in the following table.

TABLE A-23

| Cpd | From | | | Exact | [M + H]⁺ |
|---|---|---|---|---|---|
| no | amine | structure | name | mass | found |
| 383 | 271 | 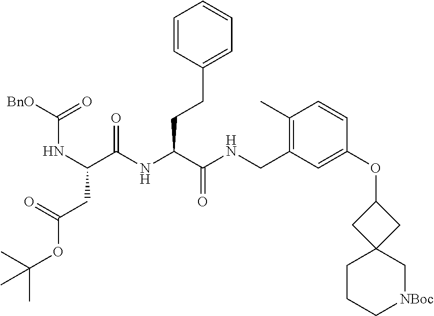 | tert-butyl 2-(3-((5S,8S)-5- (2-(tert-butoxy)-2- oxoethyl)-3,6,9-trioxo-8- phenethyl-1-phenyl-2-oxa- 4,7,10-triazaundecan-11- yl)-4-methylphenoxy)-6- azaspiro[3.5]nonane-6- carboxylate | 814.5 | 717.5 [M + H − Boc]+ |
| 397 | 399 | 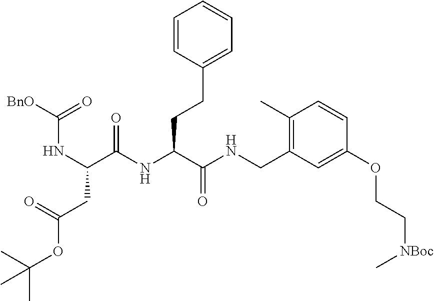 | (S)-tert-butyl 3- (((benzyloxy)carbonyl) amino)-4-(((S)- 1-((5-(2-((tert- butoxycarbonyl)(methyl) amino)ethoxy)-2- methylbenzyl)amino)-1- oxo-4-phenylbutan-2- yl)amino)-4-oxobutanoate | 826.5 | 727.5 [M + H − Boc]+ |
| 406 | 416 | 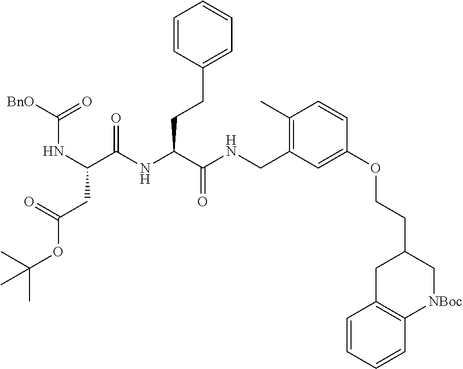 [SMILES: Cc1ccc(OCCC2Cc3ccccc3N(C(=O)OC(C)(C)C)C2)cc1CNC(=O)[C@H](CCc1ccccc1)NC(=O)[C@H](CC(=O)OC(C)(C)C)NC(=O)OCc1ccccc1] | tert-butyl 3-(2-(3-((5S,8S)- 5-(2-(tert-butoxy)-2- oxoethyl)-3,6,9-trioxo-8- phenethyl-1-phenyl-2-oxa- 4,7,10-triazaundecan-11- yl)-4- methylphenoxy)ethyl)-3,4- dihydroquinoline-1(2H)- carboxylate | 862.5 | 863.3 |
| 407 | 417 | 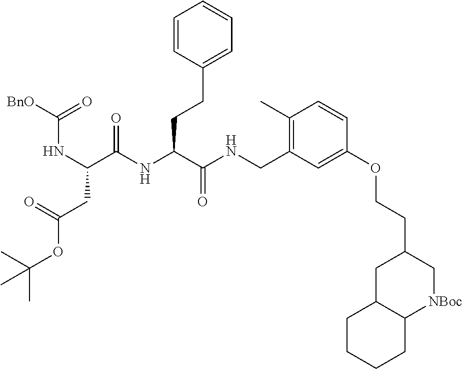 [SMILES: Cc1ccc(OCCC2CC3CCCCC3N(C(=O)OC(C)(C)C)C2)cc1CNC(=O)[C@H](CCc1ccccc1)NC(=O)[C@H](CC(=O)OC(C)(C)C)NC(=O)OCc1ccccc1] | tert-butyl 3-(2-(3-((5S,8S)- 5-(2-(tert-butoxy)-2- oxoethyl)-3,6,9-trioxo-8- phenethyl-1-phenyl-2-oxa- 4,7,10-triazaundecan-11- yl)-4- octamethylphenoxy)ethyl) hydroquinoline-1 (2H)- carboxylate | 868.5 | 869.3 |
| 410 | 411 | 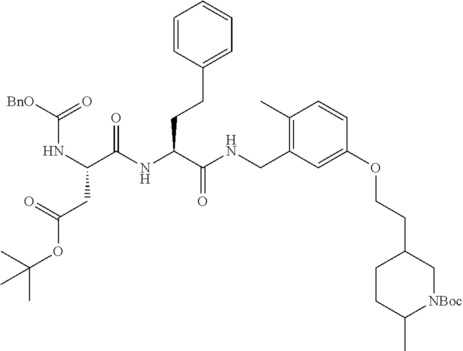 [SMILES: Cc1ccc(OCCC2CCC(C)N(C(=O)OC(C)(C)C)C2)cc1CNC(=O)[C@H](CCc1ccccc1)NC(=O)[C@H](CC(=O)OC(C)(C)C)NC(=O)OCc1ccccc1] | tert-butyl 5-(2-(3-((5S,8S)- 5-(2-(tert-butoxy)-2- oxoethyl)-3,6,9-trioxo-8- phenethyl-1-phenyl-2-oxa- 4,7,10-triazaundecan-11- yl)-4- methylphenoxy)ethyl)-2- methylpiperidine-1- carboxylate | 828.5 | 729.6 [M + H − Boc]+ |
| 427 | 426 | 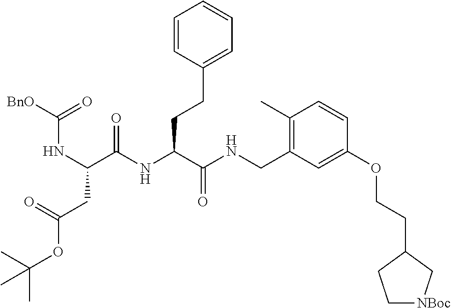 [SMILES: Cc1ccc(OCCC2CCN(C(=O)OC(C)(C)C)C2)cc1CNC(=O)[C@H](CCc1ccccc1)NC(=O)[C@H](CC(=O)OC(C)(C)C)NC(=O)OCc1ccccc1] | tert-butyl 3-(2-(3- ((5S,8S)- 5-(2-(tert-butoxy)-2- oxoethyl)-3,6,9-trioxo-8- phenethyl-1-phenyl-2-oxa- 4,7,10-triazaundecan-11- yl)-4- methylphenoxy)ethyl) pyrrolidine-1-carboxylate | 800.4 | 701.4 [M + H − Boc]+ |
| 432 | 430 | 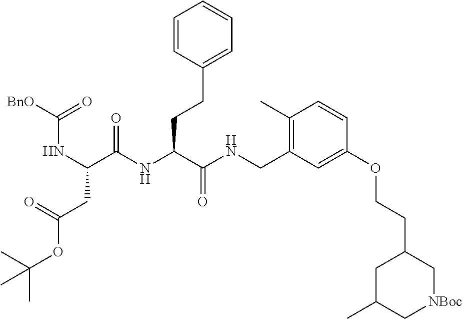 [SMILES: Cc1ccc(OCCC2CC(C)CN(C(=O)OC(C)(C)C)C2)cc1CNC(=O)[C@H](CCc1ccccc1)NC(=O)[C@H](CC(=O)OC(C)(C)C)NC(=O)OCc1ccccc1] | tert-butyl 3-(2-(3-((5S,8S)- 5-(2-(tert-butoxy)-2- oxoethyl)-3,6,9-trioxo-8- phenethyl-1-phenyl-2-oxa- 4,7,10-triazaundecan-11- yl)-4- methylphenoxy)ethyl)-5- methylpiperidine-1- carboxylate | 828.5 | 829.4 |

### (S)-4-(((S)-1-((5-(6-azaspiro[3.5]nonan-2-yloxy)-2-methylbenzyl)amino)-1-oxo-4-phenylbutan-2-yl)amino)-3-(((benzyloxy)carbonyl)amino)-4-oxobutanoic acid (384)

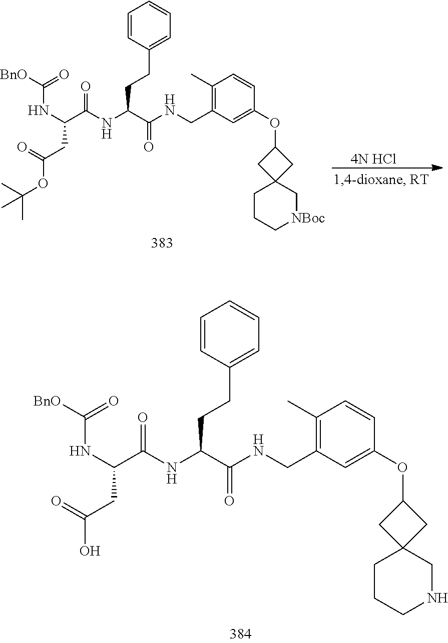

Protecting groups were cleaved from 900 mg compound 383 according to general procedure D stirring compounds in 10 ml 4 N HCl/1,4-dioxane at room temperature. After deprotection was complete solvent was removed and the crude dissolved in acetonitrile and concentrated twice. Product used without further purification. Additional examples are disclosed in the following table.

TABLE A-24

| Cpd | From | | | Exact | [M + H]⁺ |
|---|---|---|---|---|---|
| no | cpd | structure | name | mass | found |
| 384 | 383 | 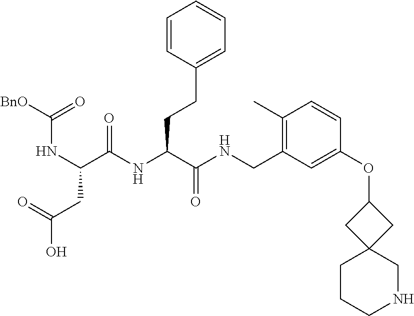 [SMILES: Cc1ccc(OC2CC3(CCCNC3)C2)cc1CNC(=O)[C@H](CCc1ccccc1)NC(=O)[C@H](CC(=O)O)NC(=O)OCc1ccccc1] | (S)-4-(((S)-1-((5-(6- azaspiro[3.5]nonan-2-yloxy)- 2-methylbenzyl)amino)-1- oxo-4-phenylbutan-2- yl)amino)-3- (((benzyloxy)carbonyl)amino)- 4-oxobutanoic acid | 670.3 | 671.4 |
| 398 | 397 | 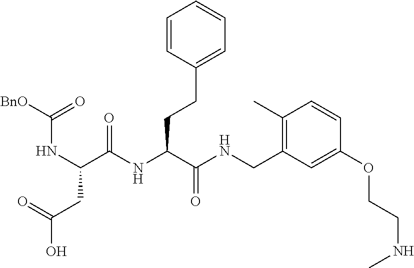 [SMILES: CNCCOc1ccc(C)c(CNC(=O)[C@H](CCc2ccccc2)NC(=O)[C@H](CC(=O)O)NC(=O)OCc2ccccc2)c1] | (S)-3- (((benzyloxy)carbonyl)amino)- 4-(((S)-1-((2-methyl-5-(2- (methylamino)ethoxy)benzyl) amino)-1-oxo-4-phenylbutan- 2-yl)amino)-4-oxobutanoic acid | 604.3 | |
| 408 | 406 | 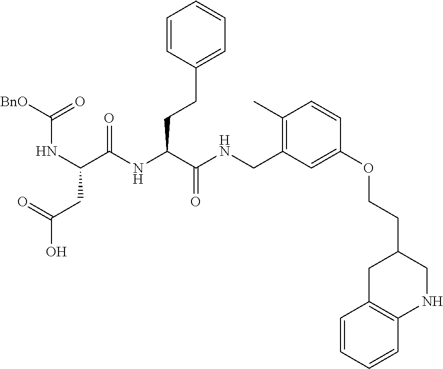 [SMILES: Cc1ccc(OCCC2CNc3ccccc3C2)cc1CNC(=O)[C@H](CCc1ccccc1)NC(=O)[C@H](CC(=O)O)NC(=O)OCc1ccccc1] | (3S)-3- (((benzyloxy)carbonyl)amino)- 4-(((2S)-1-((2-methyl-5-(2- (1,2,3,4-tetrahydroquinolin-3- yl)ethoxy)benzyl)amino)-1- oxo-4-phenylbutan-2- yl)amino)-4-oxobutanoic acid | 706.3 | 707.3 |
| 409 | 407 | 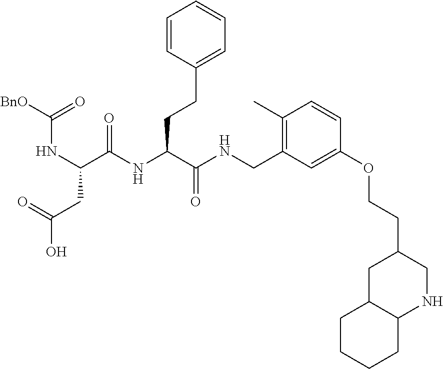 [SMILES: Cc1ccc(OCCC2CNC3CCCCC3C2)cc1CNC(=O)[C@H](CCc1ccccc1)NC(=O)[C@H](CC(=O)O)NC(=O)OCc1ccccc1] | (3S)-3- (((benzyloxy)carbonyl) amino)-4-(((2S)-1-((5-(2- (decahydroquinolin-3- yl)ethoxy)-2- methylbenzyl)amino)-1-oxo- 4-phenylbutan-2-yl)amino)-4- oxobutanoic acid | 712.4 | 713.3 |
| 418 | 410 | 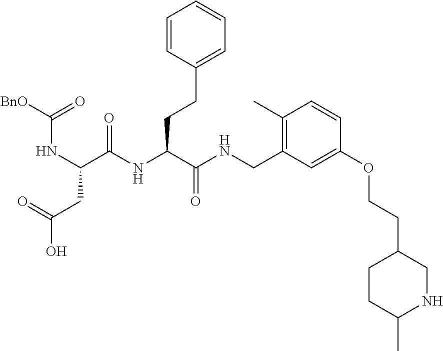 [SMILES: Cc1ccc(OCCC2CCC(C)NC2)cc1CNC(=O)[C@H](CCc1ccccc1)NC(=O)[C@H](CC(=O)O)NC(=O)OCc1ccccc1] | (3S)-3- (((benzyloxy)carbonyl)amino)- 4-(((2S)-1-((2-methyl-5-(2-(6- methylpiperidin-3- yl)ethoxy)benzyl)amino)-1- oxo-4-phenylbutan-2- yl)amino)-4-oxobutanoic acid | 672.4 | 673.3 |
| 428 | 427 | 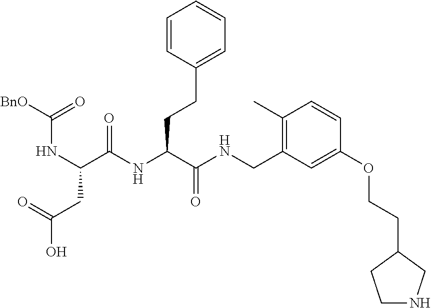 [SMILES: Cc1ccc(OCCC2CCNC2)cc1CNC(=O)[C@H](CCc1ccccc1)NC(=O)[C@H](CC(=O)O)NC(=O)OCc1ccccc1] | (3S)-3- (((benzyloxy)carbonyl)amino)- 4-(((2S)-1-((2-methyl-5-(2- (pyrrolidin-3- yl)ethoxy)benzyl)amino)-1- oxo-4-phenylbutan-2- yl)amino)-4-oxobutanoic acid | 644.3 | 645.2 |
| 433 | 432 | 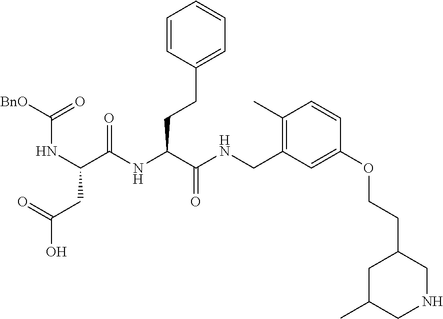 [SMILES: Cc1ccc(OCCC2CNCC(C)C2)cc1CNC(=O)[C@H](CCc1ccccc1)NC(=O)[C@H](CC(=O)O)NC(=O)OCc1ccccc1] | (3S)-3- (((benzyloxy)carbonyl)amino)- 4-(((2S)-1-((2-methyl-5-(2-(5- methylpiperidin-3- yl)ethoxy)benzyl)amino)-1- oxo-4-phenylbutan-2- yl)amino)-4-oxobutanoic acid | 672.4 | 673.4 |

### benzyl ((7S,10S)-3⁴-methyl-6,9,12-trioxo-7-phenethyl-2-oxa-5,8-diaza-13(3,1)-piperidina-3(1,3)-benzena-1(1,3)-cyclobutanatridecaphane-10-yl)carbamate (300)

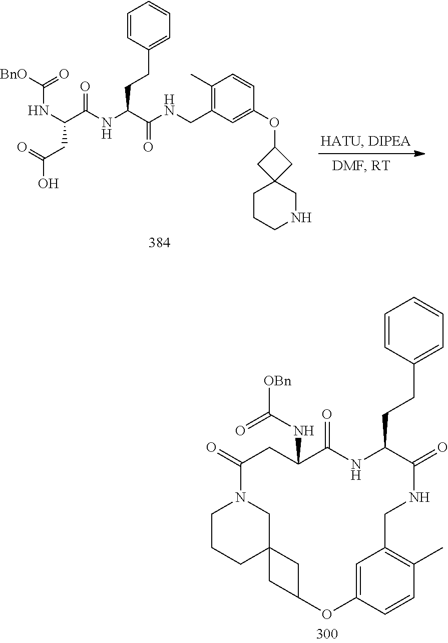 [SMILES: Cc1ccc(OC2CC3(CCCNC3)C2)cc1CNC(=O)[C@H](CCc1ccccc1)NC(=O)[C@H](CC(=O)O)NC(=O)OCc1ccccc1 ; Cc1ccc2cc1CNC(=O)[C@H](CCc1ccccc1)NC(=O)[C@@H](NC(=O)OCc1ccccc1)CC(=O)N1CCCC3(CC(C3)O2)C1]

Macrocyclization was achieved according to general procedure A with 93 mg amino acid 384. Reaction mixtures were diluted with some methanol and purified via HPLC. Additional examples are disclosed in the following table.

TABLE A-25

| Cpd | From | | | Exact | [M + H]⁺ |
|---|---|---|---|---|---|
| no | cpd | structure | name | mass | found |
| 300 | 384 | 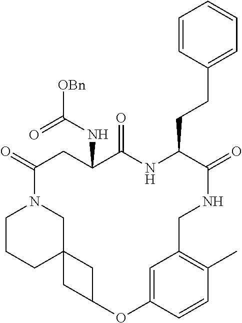 [SMILES: Cc1ccc2cc1CNC(=O)[C@H](CCc1ccccc1)NC(=O)[C@@H](NC(=O)OCc1ccccc1)CC(=O)N1CCCC3(CC(C3)O2)C1] | benzyl ((7S,10S)-3⁴-methyl- 6,9,12-trioxo-7-phenethyl-2- oxa-5,8-diaza-13(3,1)- piperidina-3(1,3)-benzena- 1(1,3)- cyclobutanatridecaphane- 10-yl)carbamate | 652.3 | 653.4 |
| 312 | 398 | 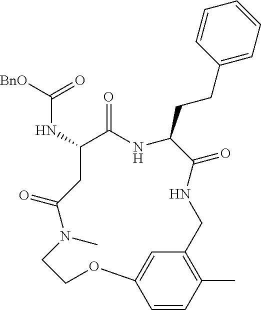 [SMILES: Cc1ccc2cc1CNC(=O)[C@H](CCc1ccccc1)NC(=O)[C@@H](NC(=O)OCc1ccccc1)CC(=O)N(C)CCO2] | benzyl ((8S,11S)-1⁴,5- dimethyl-6,9,12-trioxo-11- phenethyl-2-oxa-5,10,13- triaza-1(1,3)- benzenacyclotetradecaphane- 8-yl)carbamate | 586.3 | 587.3 |
| 313 | 408 | 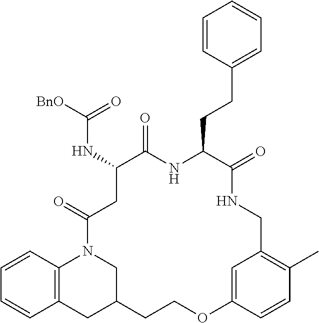 [SMILES: Cc1ccc2cc1CNC(=O)[C@H](CCc1ccccc1)NC(=O)[C@@H](NC(=O)OCc1ccccc1)CC(=O)N1CC(CCO2)Cc2ccccc21] | benzyl ((9S, 12S)-5⁴-methyl- 8,11,14-trioxo-9-phenethyl- 11,12,13,14-tetrahydro-4- oxa-7,10-diaza-1(3,1)- quinolina-5(1,3)- benzenacyclotetradecaphane- 12-yl)carbamate | 688.3 | 689.4 |
| 314 | 409 | 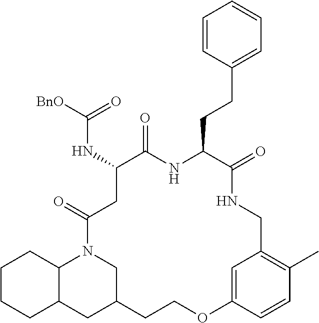 [SMILES: Cc1ccc2cc1CNC(=O)[C@H](CCc1ccccc1)NC(=O)[C@@H](NC(=O)OCc1ccccc1)CC(=O)N1CC(CCO2)CC2CCCCC21] | benzyl ((9S,12S)-5⁴-methyl- 8,11,14-trioxo-9-phenethyl- 11,12,13,14,14a,15,16,17,18,18a- decahydro-4-oxa- 7,10-diaza-1(3,1)-quinolina- 5(1,3)- benzenacyclotetradecaphane- 12-yl)carbamate | 694.4 | 965.4 |
| 315 | 418 | 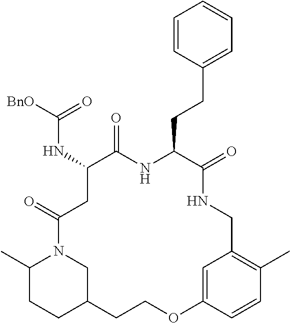 | benzyl ((9S,12S)-1⁶,5⁴- dimethyl-8,11,14-trioxo-9- phenethyl-4-oxa-7,10-diaza- 1(3,1)-piperidina-5(1,3)- benzenacyclotetradecaphane- 12-yl)carbamate | 654.3 | 655.4 |
| 325 | 428 | 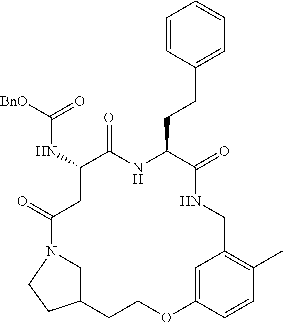 [SMILES: Cc1ccc2cc1CNC(=O)[C@H](CCc1ccccc1)NC(=O)[C@@H](NC(=O)OCc1ccccc1)CC(=O)N1CCC(CCO2)C1] | benzyl ((9S,12S)-5⁴-methyl- 8,11,14-trioxo-9-phenethyl- 4-oxa-7,10-diaza-1(3,1)- pyrrolidina-5(1,3)- benzenacyclotetradecaphane- 12-yl)carbamate | 626.3 | 627.3 |
| 337 | 433 | 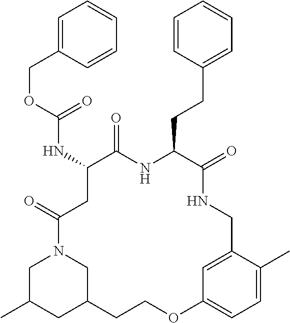 | benzyl ((9S,12S)-1⁵,5⁴- dimethyl-8,11,14-trioxo-9- phenethyl-4-oxa-7,10-diaza- 1(3,1)-piperidina-5(1,3)- benzenacyclotetradecaphane- 12-yl)carbamate | 654.3 | 655.4 |

### Example A-25: Preparation of Macrocyclic Compound 211

### tert-butyl 3-(2-(3-cyano-4-fluorophenoxy)ethyl)piperidine-1-carboxylate (576)

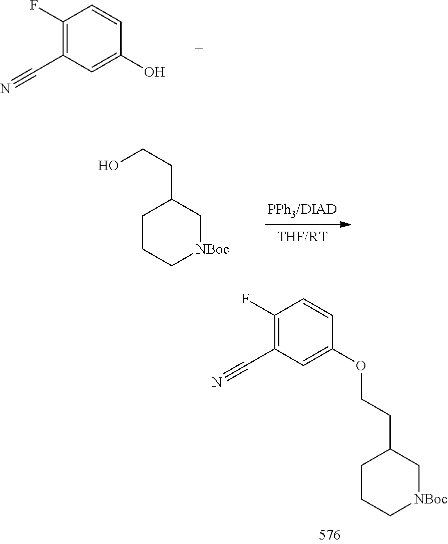 [SMILES: N#Cc1cc(O)ccc1F ; CC(C)(C)OC(=O)N1CCCC(CCO)C1 ; CC(C)(C)OC(=O)N1CCCC(CCOc2ccc(F)c(C#N)c2)C1]

1.03 g tert-butyl 3-(2-hydroxyethyl)piperidine-1-carboxylate, 500 mg 2-fluoro-5-hydroxybenzonitrile and 1.17 g PPh₃ were dissolved in THF. 879 μl DIAD in 5 ml THF were added and the mixture was stirred at room temperature for 2h. Saturated NaHCO₃ solution was added and the mixture was extracted with DCM. The organic phase was dried over MgSO₄ and volatiles were removed. Purification was achieved by normal phase column chromatography (silica, cyclohexane/ethyl acetate gradient).

### tert-butyl 3-(2-(3-(aminomethyl)-4-fluorophenoxy)ethyl)piperidine-1-carboxylate (200)

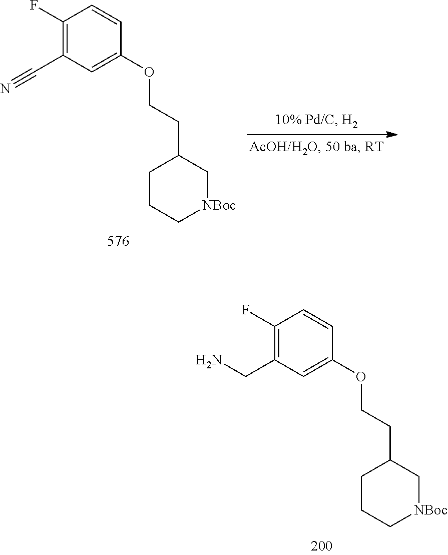 [SMILES: CC(C)(C)OC(=O)N1CCCC(CCOc2ccc(F)c(C#N)c2)C1 ; CC(C)(C)OC(=O)N1CCCC(CCOc2ccc(F)c(CN)c2)C1]

1.02 g nitrile were dissolved in 36 ml acetic acid and 5 ml water. Hydrogenation to amine 200 was achieved with 706 mg 10% Pd/C under 50 bar hydrogen at room temperature. The mixture was filtered over a pad of celite and washed with MeOH. The solvent was removed under reduced pressure. The residue was dissolved in ethyl acetate and washed with NaHCO₃-solution (3×). Combined organic layers were dried over MgSO₄, filtered off, and concentrated under reduced pressure. The residue was pure enough for the next reactions.

Formula: C₁₉H₂₉FN₂O₃, exact mass: 352.2.3, found: 353.0 [M+H]⁺

### tert-butyl 3-(2-(3-(((S)-2-(((benzyloxy)carbonyl)amino)-4-phenylbutanamido)methyl)-4-fluorophenoxy)ethyl)piperidine-1-carboxylate (201)

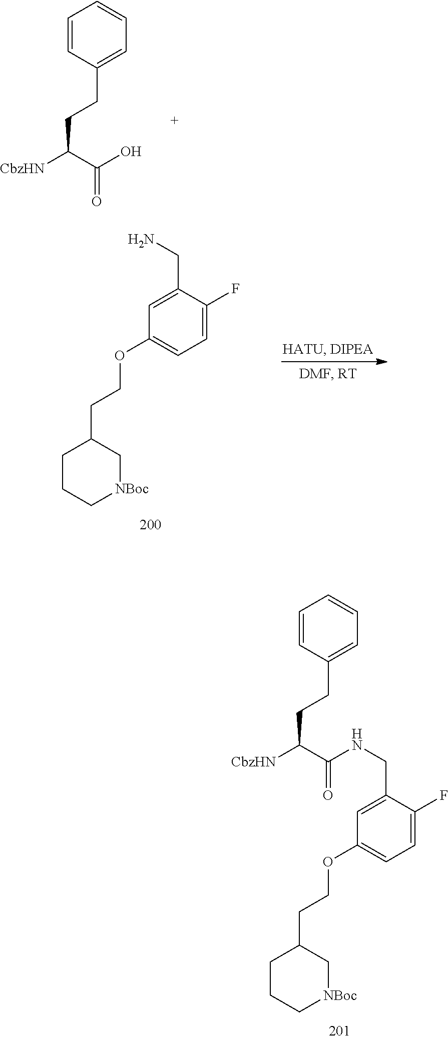 [SMILES: O=C(N[C@@H](CCc1ccccc1)C(=O)O)OCc1ccccc1 ; CC(C)(C)OC(=O)N1CCCC(CCOc2ccc(F)c(CN)c2)C1 ; CC(C)(C)OC(=O)N1CCCC(CCOc2ccc(F)c(CNC(=O)[C@H](CCc3ccccc3)NC(=O)OCc3ccccc3)c2)C1]

The amide bond was formed according to general procedure B with 615 mg amine 200 and 820 mg Cbz-hPhe-OH. Purification was achieved by normal phase column chromatography (silica, cyclohexane/ethyl acetate gradient).

Formula: C₃₇H₄₆FN₃O₆, exact mass: 647.3, found: 648.6 [M+H]⁺

### tert-butyl 3-(2-(3-(((S)-2-amino-4-phenylbutanamido)methyl)-4-fluorophenoxy)ethyl)piperidine-1-carboxylate (202)

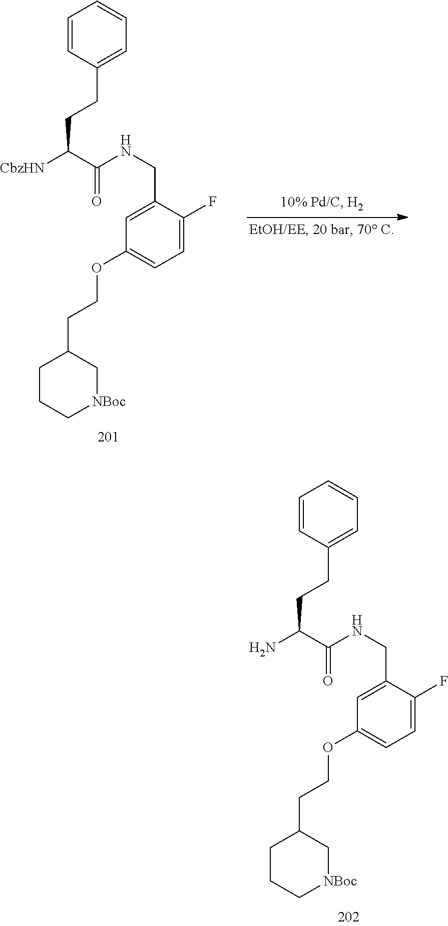 [SMILES: CC(C)(C)OC(=O)N1CCCC(CCOc2ccc(F)c(CNC(=O)[C@H](CCc3ccccc3)NC(=O)OCc3ccccc3)c2)C1 ; CC(C)(C)OC(=O)N1CCCC(CCOc2ccc(F)c(CNC(=O)[C@@H](N)CCc3ccccc3)c2)C1]

Benzyl carbamate was cleaved according to general procedure C using 10% Pd/C catalyst cartridge, 20 bar at 70° C. in a solvent mixture of ethanol/ethyl acetate. Product was purified by normal phase column chromatography (silica, cyclohexane/ethyl acetate gradient).

Formula: C₂₉H₄₀FN₃O₄, exact mass: 513.3, found: 514.3 [M+H]⁺

### tert-butyl 3-(2-(3-(((S)-2-((S)-2-acetamido-4-(tert-butoxy)-4-oxobutanamido)-4-phenylbutanamido)methyl)-4-fluorophenoxy)ethyl)piperidine-1-carboxylate (203)

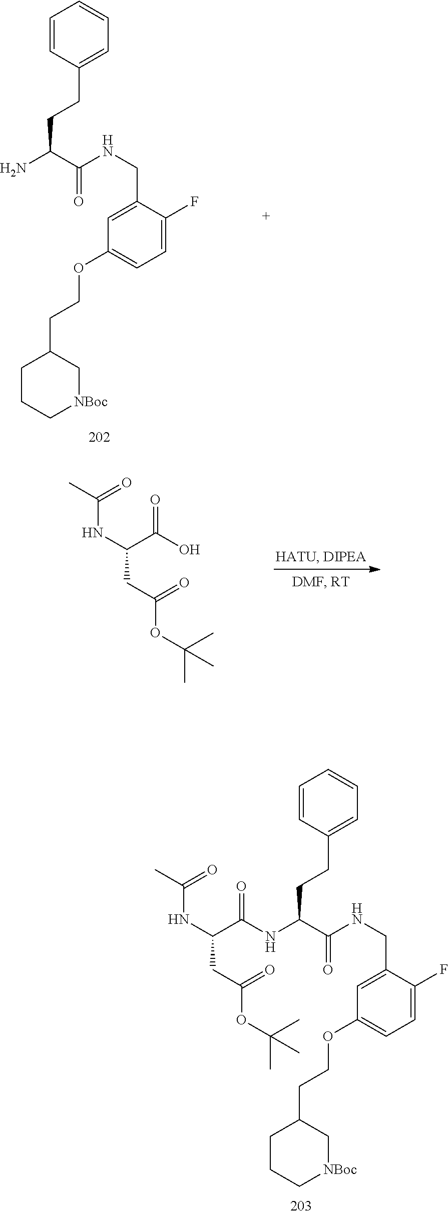 [SMILES: CC(C)(C)OC(=O)N1CCCC(CCOc2ccc(F)c(CNC(=O)[C@@H](N)CCc3ccccc3)c2)C1 ; CC(=O)N[C@@H](CC(=O)OC(C)(C)C)C(=O)O ; CC(=O)N[C@@H](CC(=O)OC(C)(C)C)C(=O)N[C@@H](CCc1ccccc1)C(=O)NCc1cc(OCCC2CCCN(C(=O)OC(C)(C)C)C2)ccc1F]

The amide bond was formed according to general procedure B with 150 mg amine 202 and 88 mg Ac-Asp(OtBu)-OH. Purification was achieved by normal phase flash chromatography.

Formula: C₃₉H₅₅FN₄O₈, exact mass: 726.4, found: 727.7 [M+H]⁺

### (3S)-3-acetamido-4-(((2S)-1-((2-fluoro-5-(2-(piperidin-3-yl)ethoxy)benzyl)amino)-1-oxo-4-phenylbutan-2-yl)amino)-4-oxobutanoic acid (204)

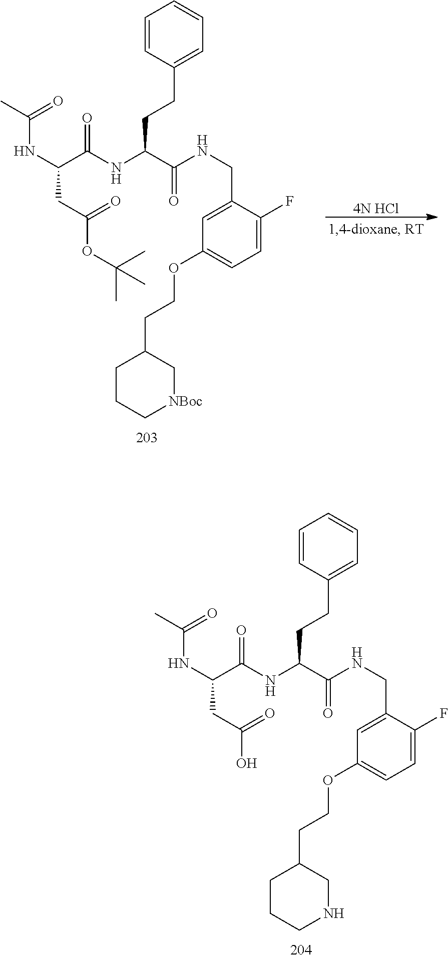

Protecting groups were cleaved from 101 mg compound 203 according to general procedure D stirring compounds in 10 ml 4 N HCl/1,4-dioxane at room temperature. After deprotection was complete solvent was removed and the crude dissolved in acetonitrile and concentrated twice. Product used without further purification

Formula: C₃₀H₃₉FN₄O₆, exact mass: 570.3, found: 571.4 [M+H]⁺

### N-((9S,12S)-5⁴-fluoro-8,11,14-trioxo-9-phenethyl-4-oxa-7,10-diaza-1(3,1)-piperidina-5(1,3)-benzenacyclotetradecaphane-12-yl)acetamide (205)

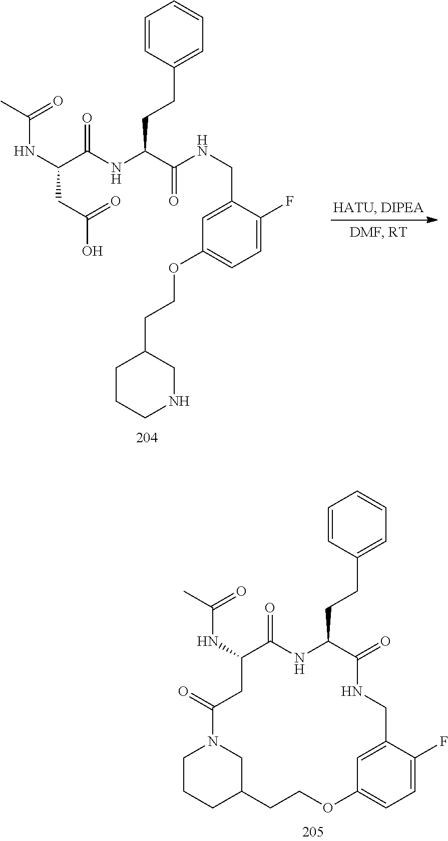 [SMILES: CC(=O)N[C@@H](CC(=O)O)C(=O)N[C@@H](CCc1ccccc1)C(=O)NCc1cc(OCCC2CCCNC2)ccc1F ; CC(=O)N[C@H]1CC(=O)N2CCCC(CCOc3ccc(F)c(c3)CNC(=O)[C@H](CCc3ccccc3)NC1=O)C2]

Macrocyclization was achieved according to general procedure A with 1.03 mmol amino acid 204. Reaction mixtures were diluted with some methanol and purified via HPLC.

Formula: C₃₀H₃₇FN₄O₅, exact mass: 552.3, found: 553.6 [M+H]⁺

### tert-butyl 3-(2-(3-(((S)-2-(((benzyloxy)carbonyl)amino)-4-phenylbutanamido)methyl)-4-methoxyphenoxy)ethyl)piperidine-1-carboxylate (207)

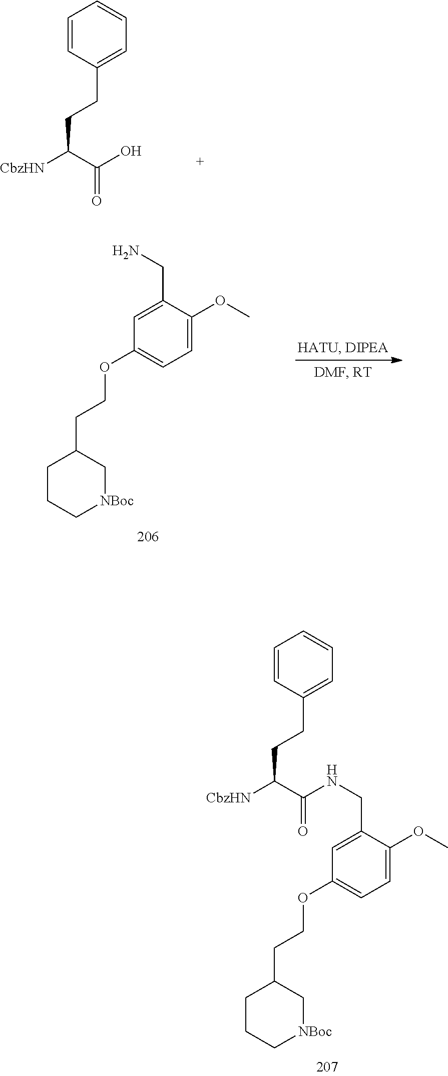 [SMILES: O=C(N[C@@H](CCc1ccccc1)C(=O)O)OCc1ccccc1 ; COc1ccc(OCCC2CCCN(C(=O)OC(C)(C)C)C2)cc1CN ; COc1ccc(OCCC2CCCN(C(=O)OC(C)(C)C)C2)cc1CNC(=O)[C@H](CCc1ccccc1)NC(=O)OCc1ccccc1]

The amide bond was formed according to general procedure B with 648 mg amine 206 and 836 mg Cbz-hPhe-OH. Purification was achieved by normal phase column chromatography (silica, cyclohexane/ethyl acetate gradient).

Formula: C₃₈H₄₉N₃O₇, exact mass: 659.4, found: 660.7 [M+H]⁺

### tert-butyl 3-(2-(3-(((S)-2-amino-4-phenylbutanamido)methyl)-4-methoxyphenoxy)ethyl) piperidine-1-carboxylate (208)

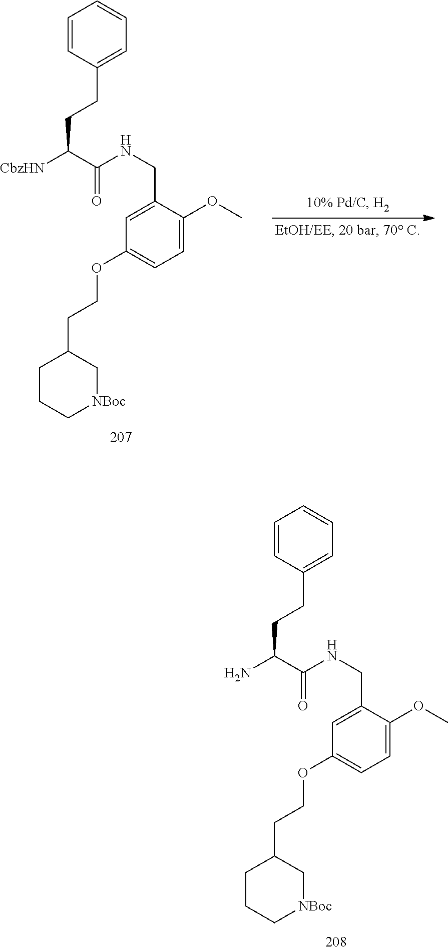 [SMILES: COc1ccc(OCCC2CCCN(C(=O)OC(C)(C)C)C2)cc1CNC(=O)[C@H](CCc1ccccc1)NC(=O)OCc1ccccc1 ; COc1ccc(OCCC2CCCN(C(=O)OC(C)(C)C)C2)cc1CNC(=O)[C@@H](N)CCc1ccccc1]

Benzyl carbamate was cleaved according to general procedure C using 10% Pd/C catalyst cartridge, 20 bar at 70° C. in a solvent mixture of ethanol/ethyl acetate. Product was purified by normal phase column chromatography (silica, cyclohexane/ethyl acetate gradient).

Formula: C₃₀H₄₃N₃O₅, exact mass: 525.3, found: 526.5 [M+H]⁺

### tert tert-butyl 3-(2-(3-(((S)-2-((S)-2-acetamido-4-(tert-butoxy)-4-oxobutanamido)-4-phenylbutanamido)methyl)-4-methoxyphenoxy)ethyl)piperidine-1-carboxylate (209)

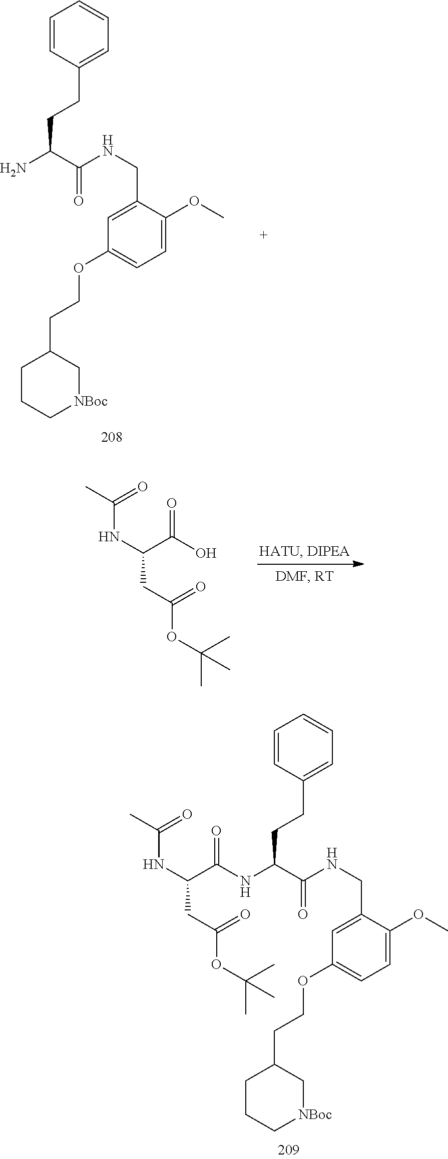 [SMILES: COc1ccc(OCCC2CCCN(C(=O)OC(C)(C)C)C2)cc1CNC(=O)[C@@H](N)CCc1ccccc1 ; CC(=O)N[C@@H](CC(=O)OC(C)(C)C)C(=O)O ; COc1ccc(OCCC2CCCN(C(=O)OC(C)(C)C)C2)cc1CNC(=O)[C@H](CCc1ccccc1)NC(=O)[C@H](CC(=O)OC(C)(C)C)NC(C)=O]

The amide bond was formed according to general procedure B with 150 mg amine 208 and 86 mg Ac-Asp(OtBu)-OH. Purification was achieved by normal phase flash chromatography.

Formula: C₄₀H₅₈N₄₀, exact mass: 738.4, found: 739.7 [M+H]⁺

### (3S)-3-acetamido-4-(((2S)-1-((2-methoxy-5-(2-(piperidin-3-yl)ethoxy)benzyl)amino)-1-oxo-4-phenylbutan-2-yl)amino)-4-oxobutanoic acid (210)

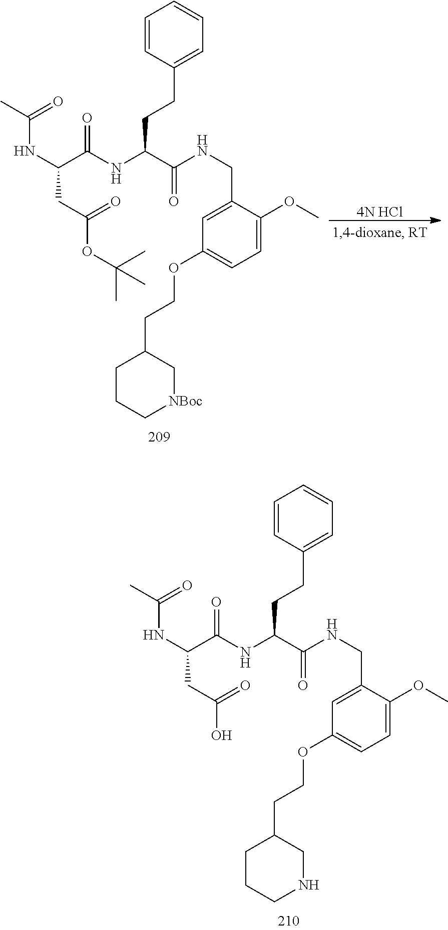

Protecting groups were cleaved from 188 mg compound 209 according to general procedure D stirring compounds in 10 ml 4 N HCl/1,4-dioxane at room temperature. After deprotection was complete solvent was removed and the crude dissolved in acetonitrile and concentrated twice. Product used without further purification

Formula: C₃₁H₄₂N₄O₇, exact mass: 582.3, found: 583.4 [M+H]⁺

### N-((9S,12S)-5⁴-methoxy-8,11,14-trioxo-9-phenethyl-4-oxa-7,10-diaza-1(3,1)-piperidina-5(1,3)-benzenacyclotetradecaphane-12-yl)acetamide (211)

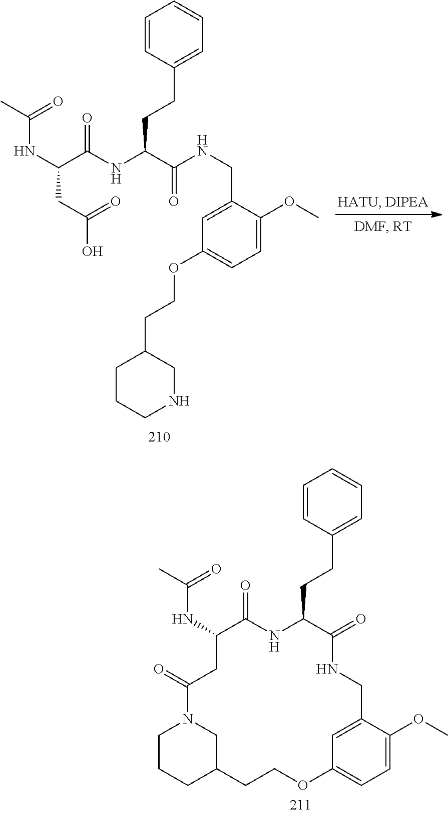

Macrocyclization was achieved according to general procedure A with 0.289 mmol amino acid 210. Reaction mixtures were diluted with some methanol and purified via HPLC.

Formula: C₃₁H₄ON₄O₆, exact mass: 564.3, found: 565.5 [M+H]⁺

### Example A-26: Preparation of Macrocyclic Compound 212, 213, 214, and 215

### (9S,12S)-5⁴-methyl-12-amino-8,11,16-trioxo-9-phenethyl-4-oxa-7,10-diaza-1(3,1)-piperidina-5(1,3)-benzenacyclohexadecaphane (212)

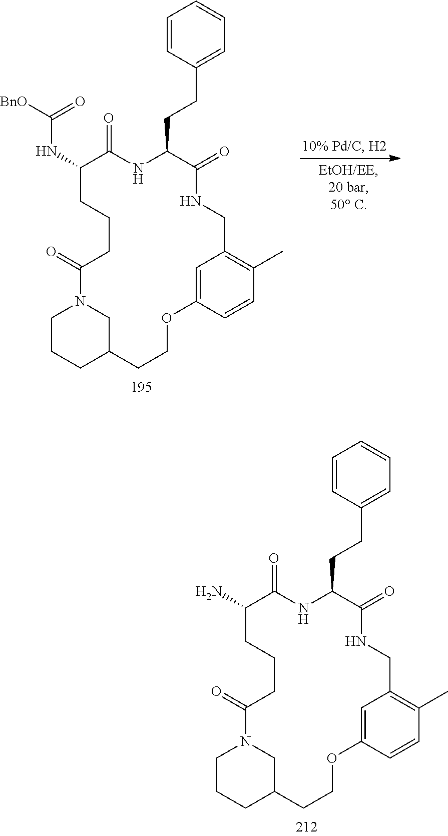 [SMILES: Cc1ccc2cc1CNC(=O)[C@H](CCc1ccccc1)NC(=O)[C@@H](NC(=O)OCc1ccccc1)CCCC(=O)N1CCCC(CCO2)C1 ; Cc1ccc2cc1CNC(=O)[C@H](CCc1ccccc1)NC(=O)[C@@H](N)CCCC(=O)N1CCCC(CCO2)C1]

Benzyl carbamate was cleaved according to general procedure C using 10% Pd/C catalyst cartridge, 20 bar at 70° C. in a solvent mixture of ethanol/ethyl acetate. Product was purified by normal phase column chromatography (silica, cyclohexane/ethyl acetate gradient).

Formula: C₃₁H₄₂N₄O₄, exact mass: 534.3, found: 535.6 [M+H]⁺

### N-((9S,12S)-5⁴-methyl-8,11,16-trioxo-9-phenethyl-4-oxa-7,10-diaza-1(3,1)-piperidina-5(1,3)-benzenacyclohexadecaphane-12-yl)acetamide (213)

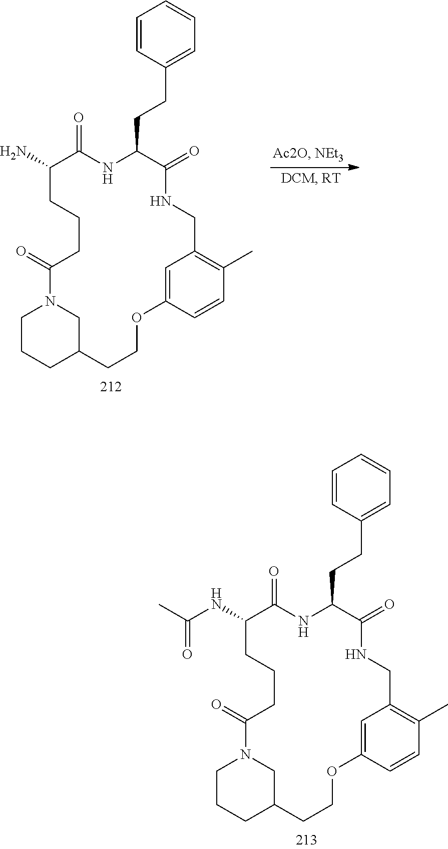 [SMILES: Cc1ccc2cc1CNC(=O)[C@H](CCc1ccccc1)NC(=O)[C@@H](N)CCCC(=O)N1CCCC(CCO2)C1 ; CC(=O)N[C@H]1CCCC(=O)N2CCCC(CCOc3ccc(C)c(c3)CNC(=O)[C@H](CCc3ccccc3)NC1=O)C2]

50 mg amine 212 and 54 μL NEt₃ were dissolved DCM and 14 μl acetic anhydride were added. After complete reaction solvent was removed and the residue dissolved in some methanol. Purification via HPLC.

Formula: C₃₃H₄₄N₄O₅, exact mass: 576.3, found: 577.6 [M+H]⁺

### (9S,12S)-12-amino-5⁴-methyl-8,11,15-trioxo-9-phenethyl-4-oxa-7,10-diaza-1(3,1)-piperidina-5(1,3)-benzenacyclopentadecaphane) (214)

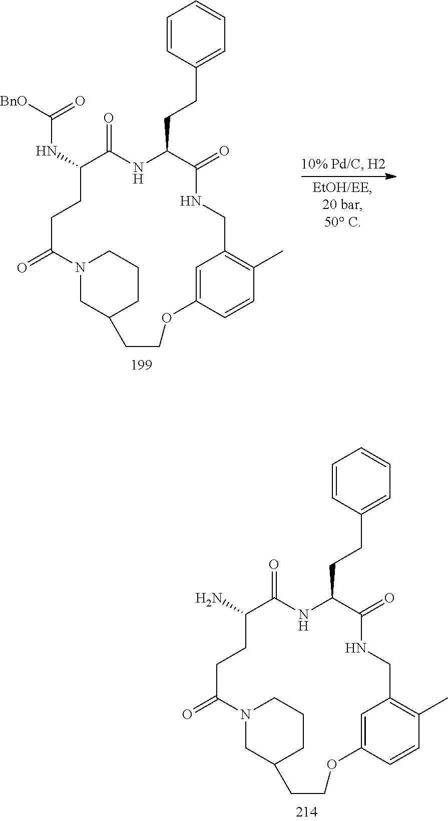 [SMILES: Cc1ccc2cc1CNC(=O)[C@H](CCc1ccccc1)NC(=O)[C@@H](NC(=O)OCc1ccccc1)CCC(=O)N1CCCC(CCO2)C1 ; Cc1ccc2cc1CNC(=O)[C@H](CCc1ccccc1)NC(=O)[C@@H](N)CCC(=O)N1CCCC(CCO2)C1]

Benzyl carbamate was cleaved from compound 199 according to general procedure C using 10% Pd/C catalyst cartridge, 20 bar at 70° C. in a solvent mixture of ethanol/ethyl acetate. Product was purified by normal phase column chromatography (silica, cyclohexane/ethyl acetate gradient).

Formula: C₃₀H₄₀N₄O₄, exact mass: 520.3, found: 521.5 [M+H]⁺

### N-((9S,12S)-5⁴-methyl-8,11,15-trioxo-9-phenethyl-4-oxa-7,10-diaza-1(3,1)-piperidina-5(1,3)-benzenacyclopentadecaphane-12-yl)acetamide (215)

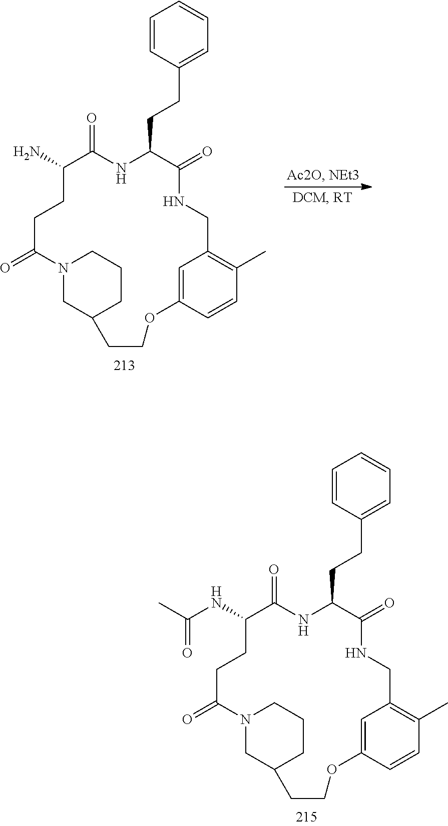 [SMILES: Cc1ccc2cc1CNC(=O)[C@H](CCc1ccccc1)NC(=O)[C@@H](N)CCC(=O)N1CCCC(CCO2)C1 ; CC(=O)N[C@H]1CCC(=O)N2CCCC(CCOc3ccc(C)c(c3)CNC(=O)[C@H](CCc3ccccc3)NC1=O)C2]

50 mg amine 213 and 54 μL NEt₃ were dissolved DCM and 14 μl acetic anhydride were added. After complete reaction solvent was removed and the residue dissolved in some methanol. Purification via HPLC.

Formula: C₃₂H₄₂N₄O₅, exact mass: 562.3, found: 563.5 [M+H]⁺

### Example A-27: Preparation of Macrocyclic Compounds 218, 220, and 293

### 2,5,8,11,14,17,20-heptaoxadocosan-22-yl ((9R,12R)-5⁴-methyl-8,11,14-trioxo-9-phenethyl-4-oxa-7,10-diaza-1(3,1)-piperidina-5(1,3)-benzenacyclotetradecaphane-12-yl)carbamate (218)

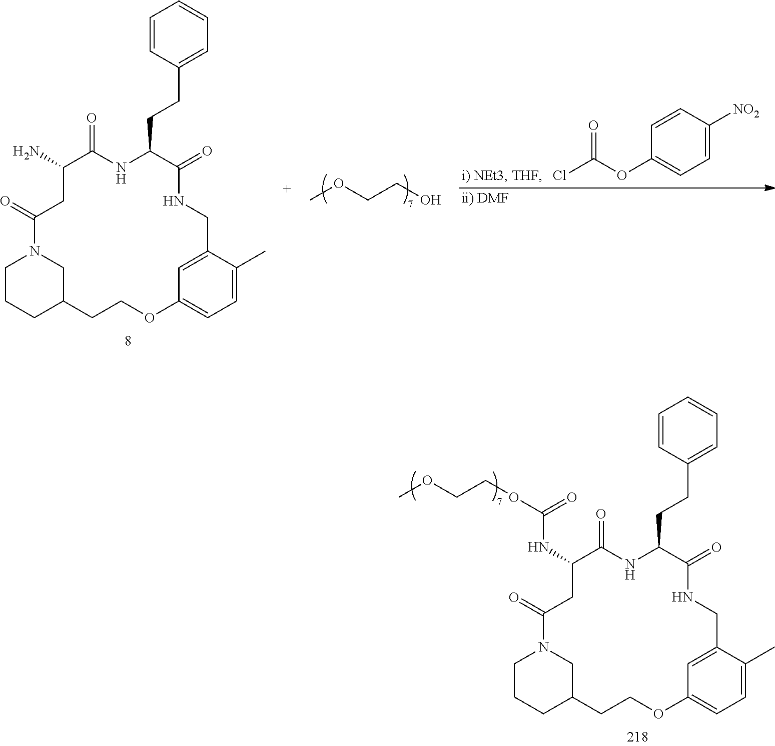

20 mg 2,5,8,11,14,17,20-heptaoxadocosan-22-ol and 24.7 μl trimethylamine in THF were cooled to 0° C. 4-Nitrophenyl chloroformate (14.2 mg) were added and the mixture was allowed to warm to room temperature. Volatiles were removed under reduced pressure and the residue dissolved in 1 ml DMF. 30 mg amine 8 were added together with 62 μl DIPEA at room temperature. After consumption of compound 8 the reaction mixture was purified by reversed phase HPLC.

Formula: C₄₅H₆₈N₄O₁₃, exact mass: 872.5, found: 873.9 [M+H]⁺

### 2-(2-(2-methoxyethoxy)ethoxy)ethyl ((9R,12R)-5⁴-methyl-8,11,14-trioxo-9-phenethyl-4-oxa-7,10-diaza-1(3,1)-piperidina-5(1,3)-benzenacyclotetradecaphane-12-yl)carbamate (220)

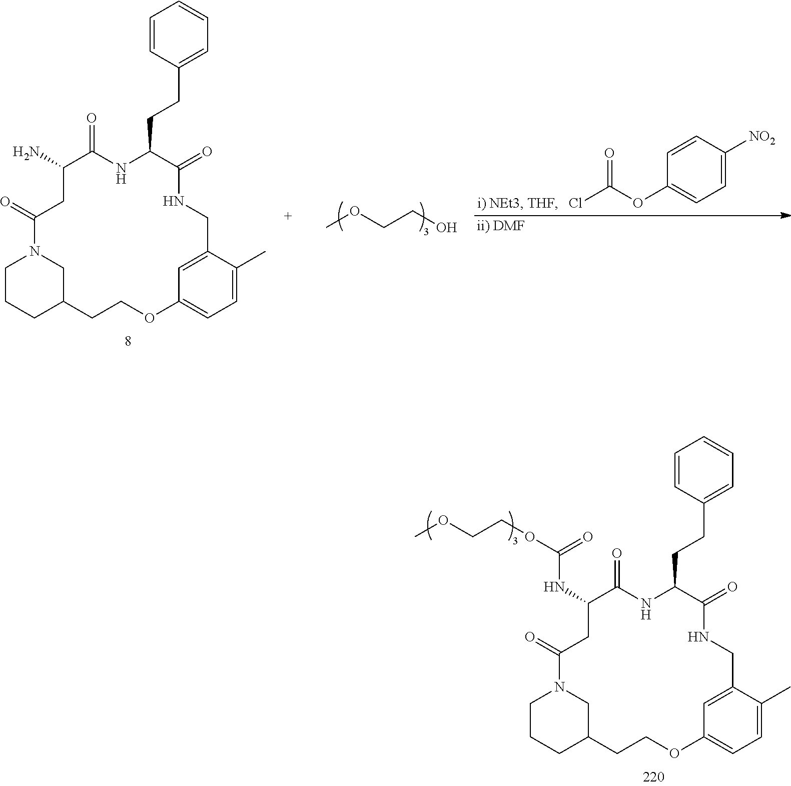

50 mg 2-(2-(2-methoxyethoxy)ethoxy)ethanol and 213 μl trimethylamine in THF were cooled to 0° C. 4-Nitrophenyl chloroformate (92 mg) were added and the mixture was allowed to warm to room temperature. Volatiles were removed under reduced pressure and the residue dissolved in 1 ml DMF. 103 mg amine 8 were added together with 212 μl DIPEA at room temperature. After consumption of compound 8 the reaction mixture was purified by reversed phase HPLC.

Formula: C₃₇H₅₂N₄O₉, exact mass: 696.4, found: 697.5 [M+H]⁺

### (3-methyloxetan-3-yl)methyl ((9S,12S)-5⁴-methyl-8,11,14-trioxo-9-phenethyl-4-oxa-7,10-diaza-1(3,1)-piperidina-5(1,3)-benzenacyclotetradecaphane-12-yl)carbamate (293)

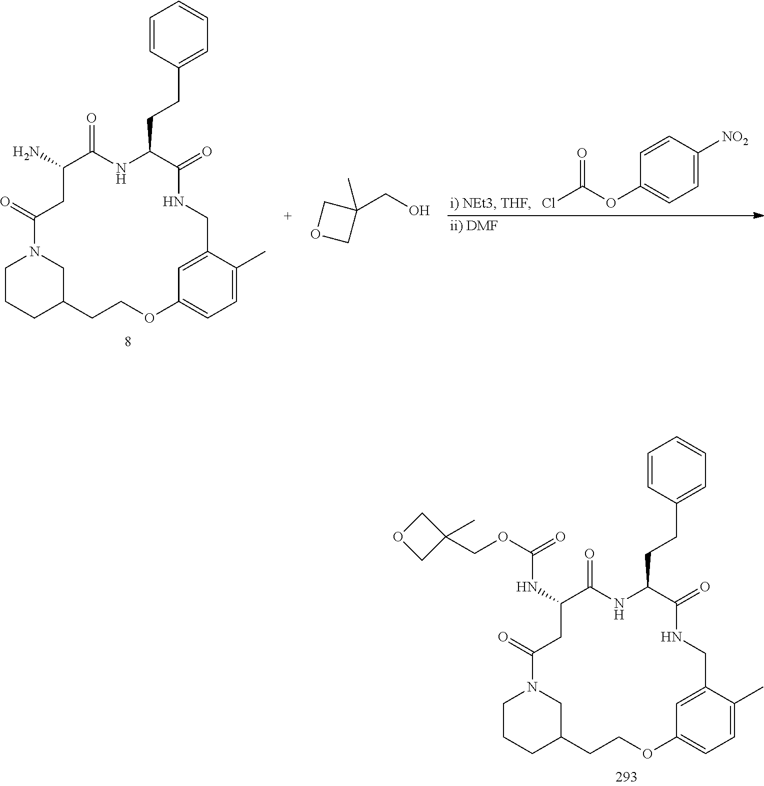

50 (3-methyloxetan-3-yl)methanol and 341 μl trimethylamine in THF were cooled to 0° C. 4-Nitrophenyl chloroformate (148 mg) were added and the mixture was allowed to warm to room temperature. Volatiles were removed under reduced pressure and the residue dissolved in 1 ml DMF. 124 mg amine 8 were added together with 742 μl DIPEA at room temperature. After consumption of compound 8 the reaction mixture was purified by reversed phase HPLC.

Formula: C₃₅H₄₆N₄O₇, exact mass: 634.3, found: 635.4 [M+H]⁺

### Example A-28: Preparation of Macrocyclic Compounds 252-261, 296 and 298

### (S)-tert-butyl 3-acetamido-4-(((S)-1-((5-(2-((tert-butoxycarbonyl)amino)ethoxy)-2-methylbenzyl)amino)-1-oxo-4-phenylbutan-2-yl)amino)-4-oxobutanoate (223)

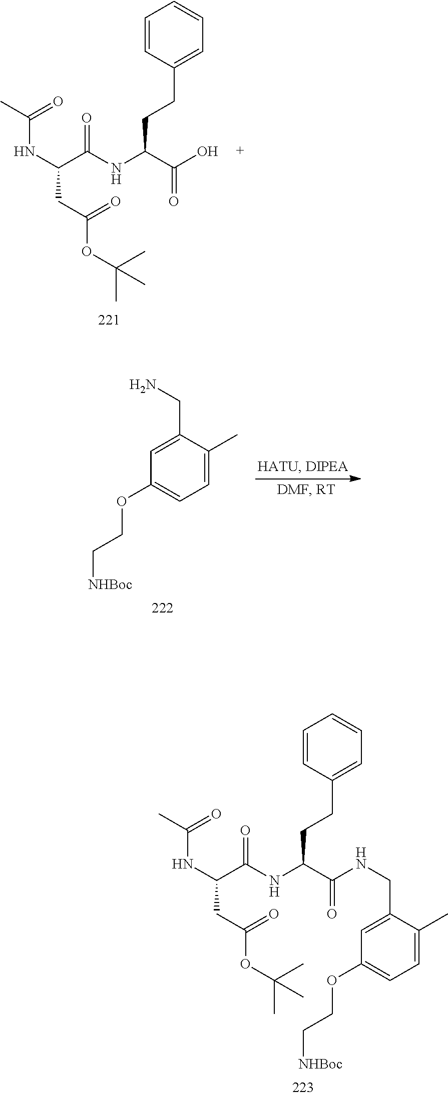 [SMILES: CC(=O)N[C@@H](CC(=O)OC(C)(C)C)C(=O)N[C@@H](CCc1ccccc1)C(=O)O ; Cc1ccc(OCCNC(=O)OC(C)(C)C)cc1CN ; CC(=O)N[C@@H](CC(=O)OC(C)(C)C)C(=O)N[C@@H](CCc1ccccc1)C(=O)NCc1cc(OCCNC(=O)OC(C)(C)C)ccc1C]

Amide couplings between benzylamine derivatives and dipeptide 221 were accomplished according to general procedure B, typically using 100 mg dipeptide 221. As an example compound 223 was synthesized from 100 mg dipeptide 221, 93 mg amine 222, 116 mg HATU and 266 μl DIPEA in DMF at room temperature. Purification was achieved by normal phase column chromatography (silica, cyclohexane/ethyl acetate gradient). Additional examples are disclosed in the following table.

TABLE A-26

| Cpd | From | | | Exact | [M + H]⁺ |
|---|---|---|---|---|---|
| no | amine | structure | name | mass | found |
| 223 | 222 | 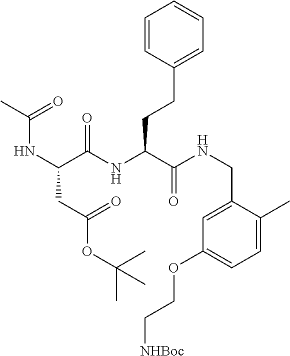 [SMILES: CC(=O)N[C@@H](CC(=O)OC(C)(C)C)C(=O)N[C@@H](CCc1ccccc1)C(=O)NCc1cc(OCCNC(=O)OC(C)(C)C)ccc1C] | (S)-tert-butyl 3-acetamido-4- (((S)-1-((5-(2-((tert- butoxycarbonyl)amino)ethoxy)- 2-methylbenzyl)amino)-1- oxo-4-phenylbutan-2- yl)amino)-4-oxobutanoate | 654.4 | 655.7 |
| 233 | 224 | 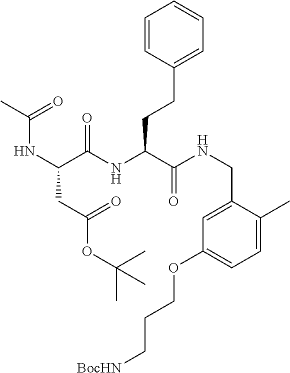 [SMILES: CC(=O)N[C@@H](CC(=O)OC(C)(C)C)C(=O)N[C@@H](CCc1ccccc1)C(=O)NCc1cc(OCCCNC(=O)OC(C)(C)C)ccc1C] | (S)-tert-butyl 3-acetamido-4- (((S)-1-((5-(3-((tert- butoxycarbonyl)amino)propoxy)- 2-methylbenzyl)amino)-1- oxo-4-phenylbutan-2- yl)amino)-4-oxobutanoate | 668.4 | 669.7 |
| 234 | 225 | 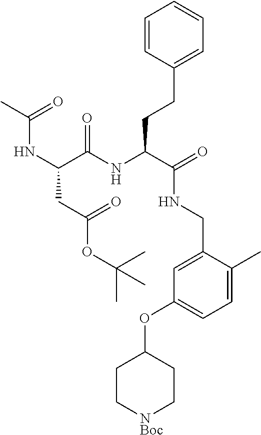 [SMILES: CC(=O)N[C@@H](CC(=O)OC(C)(C)C)C(=O)N[C@@H](CCc1ccccc1)C(=O)NCc1cc(OC2CCN(C(=O)OC(C)(C)C)CC2)ccc1C] | tert-butyl 4-(3-(((S)-2-((S)-2- acetamido-4-(tert-butoxy)-4- oxobutanamido)-4- phenylbutanamido)methyl)-4- methylphenoxy)piperidine-1- carboxylate | 694.4 | 695.7 |
| 235 | 226 | 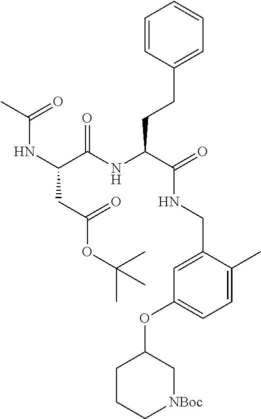 [SMILES: CC(=O)N[C@@H](CC(=O)OC(C)(C)C)C(=O)N[C@@H](CCc1ccccc1)C(=O)NCc1cc(OC2CCCN(C(=O)OC(C)(C)C)C2)ccc1C] | tert-butyl 3-(3-(((S)-2-((S)-2- acetamido-4-(tert-butoxy)-4- oxobutanamido)-4- phenylbutanamido)methyl)-4- methylphenoxy)piperidine-1- carboxylate | 694.4 | 695.7 |
| 236 | 227 | 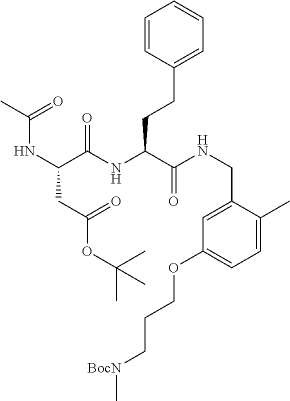 [SMILES: CC(=O)N[C@@H](CC(=O)OC(C)(C)C)C(=O)N[C@@H](CCc1ccccc1)C(=O)NCc1cc(OCCCN(C)C(=O)OC(C)(C)C)ccc1C] | (S)-tert-butyl 3-acetamido-4- (((S)-1-((5-(3-((tert- butoxycarbonyl)(methyl)amino) propoxy)-2- methylbenzyl)amino)-1-oxo- 4-phenylbutan-2-yl)amino)-4- oxobutanoate | 682.4 | 683.5 |
| 237 | 228 | 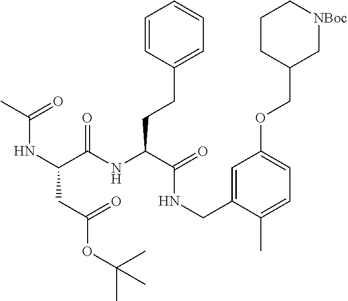 [SMILES: CC(=O)N[C@@H](CC(=O)OC(C)(C)C)C(=O)N[C@@H](CCc1ccccc1)C(=O)NCc1cc(OCC2CCCN(C(=O)OC(C)(C)C)C2)ccc1C] | tert-butyl 3-((3-(((S)-2-((S)-2- acetamido-4-(tert-butoxy)-4- oxobutanamido)-4- phenylbutanamido)methyl)-4- methylphenoxy)methyl) piperidine-1-carboxylate | 708.4 | 709.7 |
| 238 | 141 | 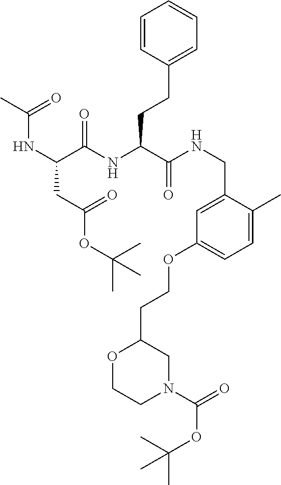 [SMILES: CC(=O)N[C@@H](CC(=O)OC(C)(C)C)C(=O)N[C@@H](CCc1ccccc1)C(=O)NCc1cc(OCCC2CN(C(=O)OC(C)(C)C)CCO2)ccc1C] | tert-butyl 2-(2-(3-(((S)-2-((S)- 2-acetamido-4-(tert-butoxy)- 4-oxobutanamido)-4- phenylbutanamido)methyl)-4- methylphenoxy)ethyl) morpholine-4-carboxylate | 724.4 | 725.7 |
| 239 | 230 | 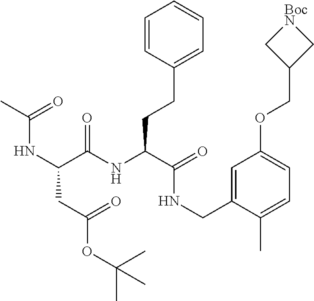 [SMILES: CC(=O)N[C@@H](CC(=O)OC(C)(C)C)C(=O)N[C@@H](CCc1ccccc1)C(=O)NCc1cc(OCC2CN(C(=O)OC(C)(C)C)C2)ccc1C] | tert-butyl 3-((3-(((S)-2-((S)-2- acetamido-4-(tert-butoxy)-4- oxobutanamido)-4- phenylbutanamido)methyl)-4- methylphenoxy)methyl) azetidine-1-carboxylate | 680.4 | 681.7 |
| 240 | 231 | 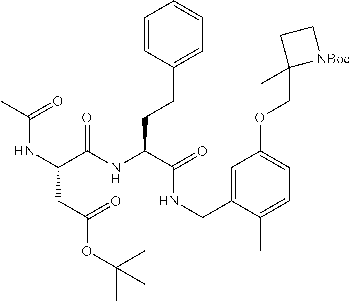 [SMILES: CC(=O)N[C@@H](CC(=O)OC(C)(C)C)C(=O)N[C@@H](CCc1ccccc1)C(=O)NCc1cc(OCC2(C)CCN2C(=O)OC(C)(C)C)ccc1C] | tert-butyl 2-((3-(((S)-2-((S)-2- acetamido-4-(tert-butoxy)-4- oxobutanamido)-4- phenylbutanamido)methyl)-4- methylphenoxy)methyl)-2- methylazetidine-1- carboxylate | 694.4 | 695.7 |
| 241 | 232 | 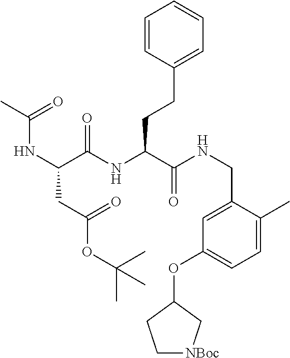 [SMILES: CC(=O)N[C@@H](CC(=O)OC(C)(C)C)C(=O)N[C@@H](CCc1ccccc1)C(=O)NCc1cc(OC2CCN(C(=O)OC(C)(C)C)C2)ccc1C] | tert-butyl 3-(3-(((S)-2-((S)-2- acetamido-4-(tert-butoxy)-4- oxobutanamido)-4- phenylbutanamido)methyl)-4- methylphenoxy)pyrrolidine-1- carboxylate | 680.4 | 681.7 |
| 376 | 270 | 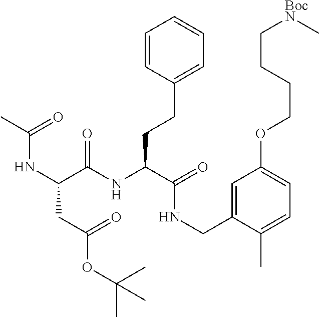 [SMILES: CC(=O)N[C@@H](CC(=O)OC(C)(C)C)C(=O)N[C@@H](CCc1ccccc1)C(=O)NCc1cc(OCCCCN(C)C(=O)OC(C)(C)C)ccc1C] | (S)-tert-butyl 3-acetamido-4- (((S)-1-((5-(4-((tert- butoxycarbonyl)(methyl) amino)butoxy)-2- methylbenzyl)amino)-1-oxo- 4-phenylbutan-2-yl)amino)-4- oxobutanoate | 568.3 | 569.4 |
| 378 | 188 | 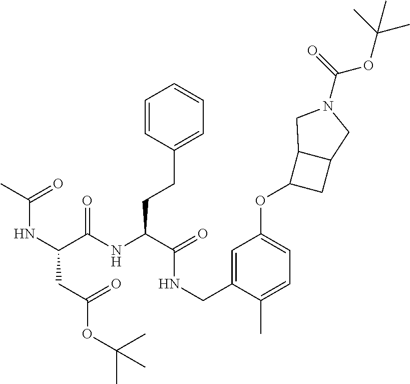 [SMILES: CC(=O)N[C@@H](CC(=O)OC(C)(C)C)C(=O)N[C@@H](CCc1ccccc1)C(=O)NCc1cc(OC2CC3CN(C(=O)OC(C)(C)C)CC32)ccc1C] | tert-butyl 6-(3-(((S)-2-((S)-2- acetamido-4-(tert-butoxy)-4- oxobutanamido)-4- phenylbutanamido)methyl)-4- methylphenoxy)-3- azabicyclo[3.2.0]heptane-3- carboxylate | 540.3 | 541.3 |

### (S)-3-acetamido-4-(((S)-1-((5-(2-aminoethoxy)-2-methylbenzyl)amino)-1-oxo-4-phenylbutan-2-yl)amino)-4-oxobutanoic acid (242)

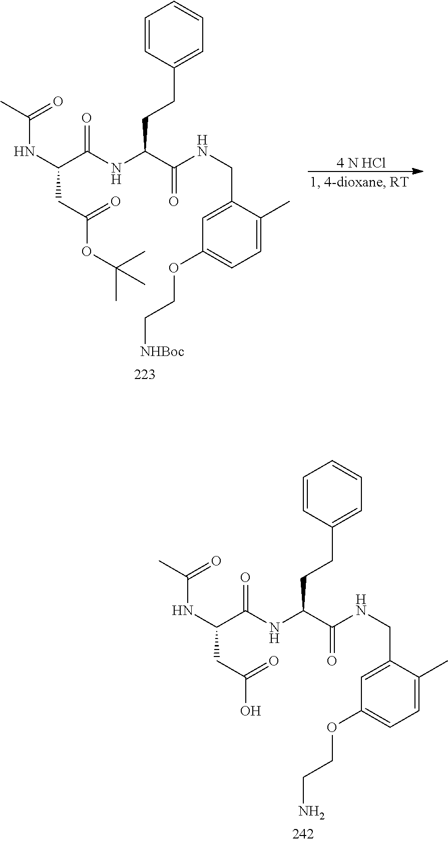

Protecting groups were cleaved from 65 mg compound 233 according to general procedure D stirring compounds in 10 ml 4 N HCl/1,4-dioxane at room temperature. After deprotection was complete solvent was removed and the crude dissolved in acetonitrile and concentrated twice. Product used without further purification.

As exemplified by compound 242 additional examples disclosed in the following table were prepared.

TABLE A-27

| Cpd | From | | | Exact | [M + H]⁺ |
|---|---|---|---|---|---|
| no | cpd | structure | name | mass | found |
| 242 | 223 | 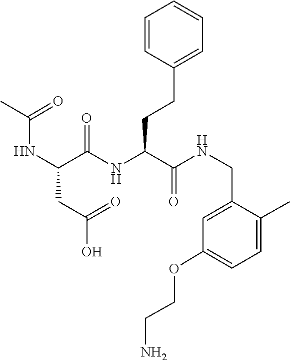 [SMILES: CC(=O)N[C@@H](CC(=O)O)C(=O)N[C@@H](CCc1ccccc1)C(=O)NCc1cc(OCCN)ccc1C] | (S)-3-acetamido-4-(((S)-1-((5- (2-aminoethoxy)-2- methylbenzyl)amino)-1-oxo- 4-phenylbutan-2-yl)amino)-4- oxobutanoic acid | 498.2 | 499.2 |
| 243 | 233 | 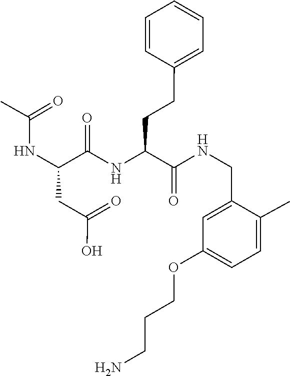 [SMILES: CC(=O)N[C@@H](CC(=O)O)C(=O)N[C@@H](CCc1ccccc1)C(=O)NCc1cc(OCCCN)ccc1C] | (S)-3-acetamido-4-(((S)-1-((5- (3-aminopropoxy)-2- methylbenzyl)amino)-1-oxo- 4-phenylbutan-2-yl)amino)-4- oxobutanoic acid | 512.3 | 513.4 |
| 244 | 234 | 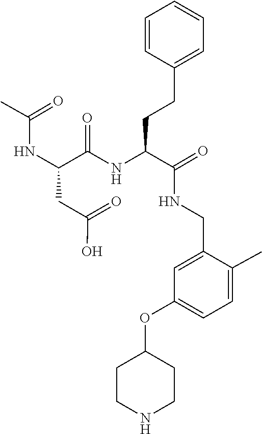 [SMILES: CC(=O)N[C@@H](CC(=O)O)C(=O)N[C@@H](CCc1ccccc1)C(=O)NCc1cc(OC2CCNCC2)ccc1C] | (S)-3-acetamido-4-(((S)-1-((2- methyl-5-(piperidin-4- yloxy)benzyl)amino)-1-oxo-4- phenylbutan-2-yl)amino)-4- oxobutanoic acid | 538.3 | 539.3 |
| 245 | 235 | 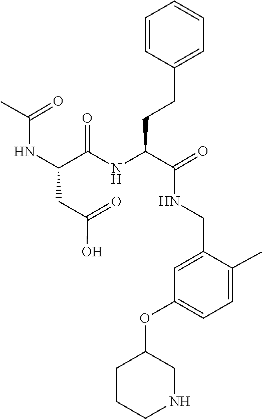 [SMILES: CC(=O)N[C@@H](CC(=O)O)C(=O)N[C@@H](CCc1ccccc1)C(=O)NCc1cc(OC2CCCNC2)ccc1C] | (3S)-3-acetamido-4-(((2S)-1- ((2-methyl-5-(piperidin-3- yloxy)benzyl)amino)-1-oxo-4- phenylbutan-2-yl)amino)-4- oxobutanoic acid | 538.3 | 539.4 |
| 246 | 236 | 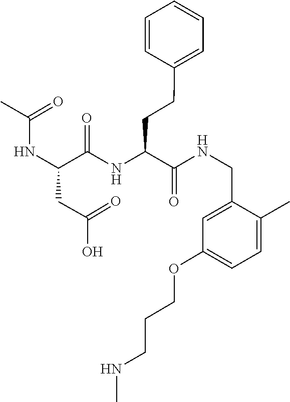 [SMILES: CNCCCOc1ccc(C)c(CNC(=O)[C@H](CCc2ccccc2)NC(=O)[C@H](CC(=O)O)NC(C)=O)c1] | (S)-3-acetamido-4-(((S)-1-((2- methyl-5-(3- (methylamino)propoxy)benzyl) amino)-1-oxo-4- phenylbutan-2-yl)amino)-4- oxobutanoic acid | 526.3 | 527.4 |
| 247 | 237 | 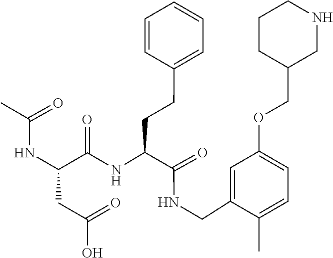 [SMILES: CC(=O)N[C@@H](CC(=O)O)C(=O)N[C@@H](CCc1ccccc1)C(=O)NCc1cc(OCC2CCCNC2)ccc1C] | (3S)-3-acetamido-4-(((2S)-1- ((2-methyl-5-(piperidin-3- ylmethoxy)benzyl)amino)-1- oxo-4-phenylbutan-2- yl)amino)-4-oxobutanoic acid | 552.3 | 553.5 |
| 248 | 238 | 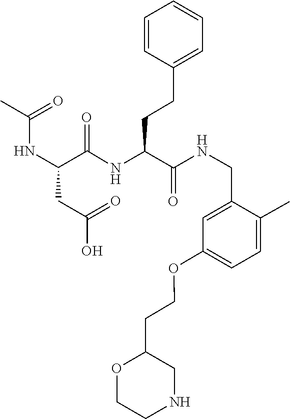 [SMILES: CC(=O)N[C@@H](CC(=O)O)C(=O)N[C@@H](CCc1ccccc1)C(=O)NCc1cc(OCCC2CNCCO2)ccc1C] | (3S)-3-acetamido-4-(((2S)-1- ((2-methyl-5-(2-(morpholin-2- yl)ethoxy)benzyl)amino)-1- oxo-4-phenylbutan-2- yl)amino)-4-oxobutanoic acid | 568.3 | 569.7 |
| 249 | 239 | 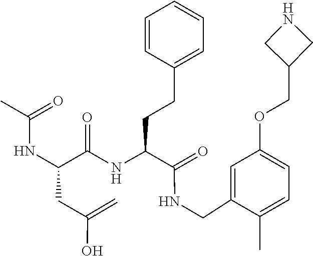 [SMILES: C=C(O)C[C@H](NC(C)=O)C(=O)N[C@@H](CCc1ccccc1)C(=O)NCc1cc(OCC2CNC2)ccc1C] | (S)-3-acetamido-4-(((S)-1-((5- (azetidin-3-ylmethoxy)-2- methylbenzyl)amino)-1-oxo- 4-phenylbutan-2-yl)amino)-4- oxobutanoic acid | 524.3 | 525.5 |
| 250 | 240 | 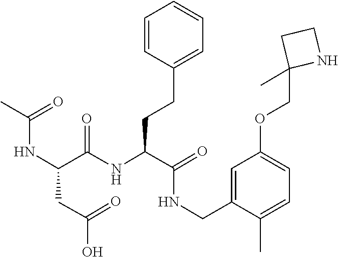 [SMILES: CC(=O)N[C@@H](CC(=O)O)C(=O)N[C@@H](CCc1ccccc1)C(=O)NCc1cc(OCC2(C)CCN2)ccc1C] | (3S)-3-acetamido-4-(((2S)-1- ((2-methyl-5-((2- methylazetidin-2- yl)methoxy)benzyl)amino)-1- oxo-4-phenylbutan-2- yl)amino)-4-oxobutanoic acid | 538.3 | 539.5 |
| 251 | 241 | 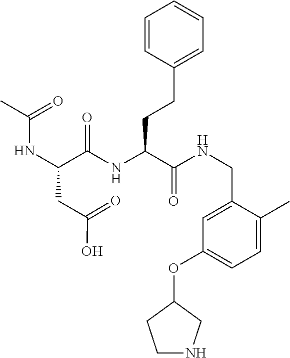 [SMILES: CC(=O)N[C@@H](CC(=O)O)C(=O)N[C@@H](CCc1ccccc1)C(=O)NCc1cc(OC2CCNC2)ccc1C] | (3S)-3-acetamido-4-(((2S)-1- ((2-methyl-5-(pyrrolidin-3- yloxy)benzyl)amino)-1-oxo-4- phenylbutan-2-yl)amino)-4- oxobutanoic acid | 524.3 | 525.3 |
| 377 | 376 | 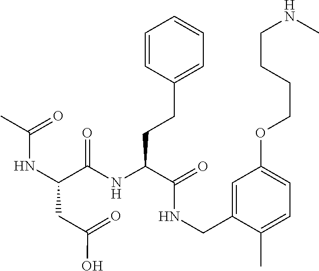 [SMILES: CNCCCCOc1ccc(C)c(CNC(=O)[C@H](CCc2ccccc2)NC(=O)[C@H](CC(=O)O)NC(C)=O)c1] | (S)-3-acetamido-4-(((S)-1-((2- methyl-5-(4- (methylamino)butoxy)benzyl) amino)-1-oxo-4-phenylbutan- 2-yl)amino)-4-oxobutanoic acid | 540.3 | 541.3 |
| 379 | 378 | 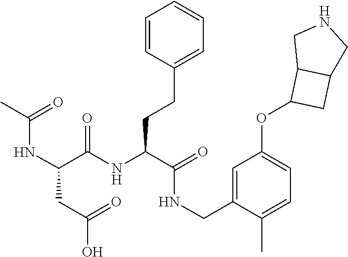 [SMILES: CC(=O)N[C@@H](CC(=O)O)C(=O)N[C@@H](CCc1ccccc1)C(=O)NCc1cc(OC2CC3CNCC32)ccc1C] | (3S)-4-(((2S)-1-((5-(3- azabicyclo[3.2.0]heptan-6- yloxy)-2- methylbenzyl)amino)-1-oxo- 4-phenylbutan-2-yl)amino)-3- acetamido-4-oxobutanoic acid | 550.3 | — |

### N-((8S,11S)-1⁴-methyl-6,9,12-trioxo-11-phenethyl-2-oxa-5,10,13-triaza-1(1,3)-benzenacyclotetradecaphane-8-yl)acetamide (252)

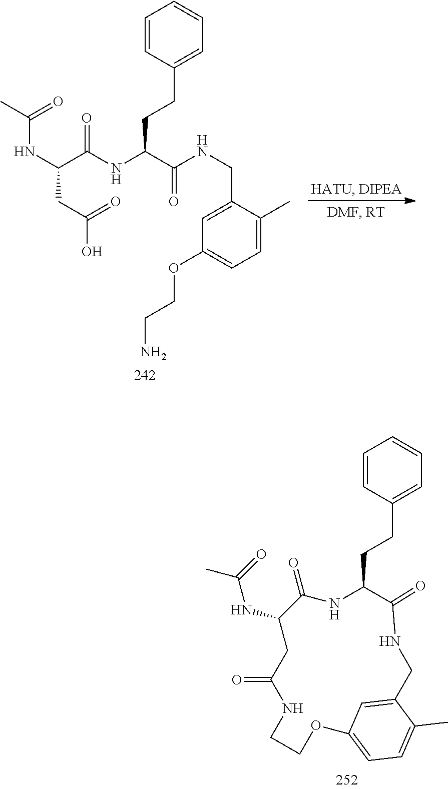

Macrocyclization was achieved according to general procedure A with 0.248 mmol amino acid 242. Reaction mixtures were diluted with some methanol and purified via HPLC. As exemplified by compound 252 additional examples disclosed in the following table were prepared.

TABLE A-28

| Cpd | From | | | Exact | [M + H]⁺ |
|---|---|---|---|---|---|
| no | cpc | structure | name | mass | found |
| 252 | 242 | 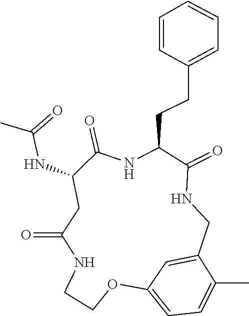 [SMILES: CC(=O)N[C@H]1CC(=O)NCCOc2ccc(C)c(c2)CNC(=O)[C@H](CCc2ccccc2)NC1=O] | N-((8S,11S)-1⁴-methyl-6,9,12- trioxo-11-phenethyl-2-oxa- 5,10,13-triaza-1(1,3)- benzenacyclotetradecaphane- 8-yl)acetamide | 480.2 C₂₆H₃₂N₄O₅ | 481.4 |
| 253 | 243 | 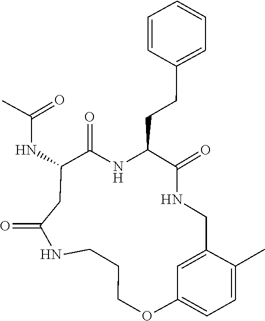 [SMILES: CC(=O)N[C@H]1CC(=O)NCCCOc2ccc(C)c(c2)CNC(=O)[C@H](CCc2ccccc2)NC1=O] | N-((9S,12S)-1⁴-methyl- 7,10,13-trioxo-12-phenethyl-2- oxa-6,11,14-triaza-1(1,3)- benzenacyclopentadecaphane- 9-yl)acetamide | 494.3 C₂₇H₃₄N₄O₅ | 495.4 |
| 254 | 244 | 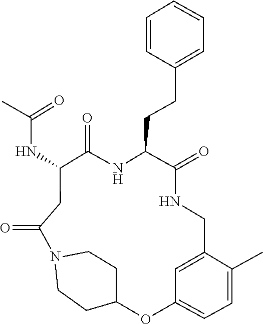 [SMILES: CC(=O)N[C@H]1CC(=O)N2CCC(CC2)Oc2ccc(C)c(c2)CNC(=O)[C@H](CCc2ccccc2)NC1=O] | N-((7S,10S)-3⁴-methyl-6,9,12- trioxo-7-phenethyl-2-oxa-5,8- diaza-1(4,1)-piperidina-3(1,3)- benzenacyclododecaphane- 10-yl)acetamide | 520.3 C₂₉H₃₆N₄O₅ | 521.4 |
| 255 | 245 | 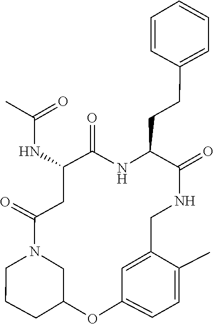 [SMILES: CC(=O)N[C@H]1CC(=O)N2CCCC(C2)Oc2ccc(C)c(c2)CNC(=O)[C@H](CCc2ccccc2)NC1=O] | N-((7S,10S)-3⁴-methyl-6,9,12- trioxo-7-phenethyl-2-oxa-5,8- diaza-1(3,1)-piperidina-3(1,3)- benzenacyclododecaphane- 10-yl)acetamide | 520.3 C₂₉H₃₆N₄O₅ | 521.4 |
| 256 | 246 | 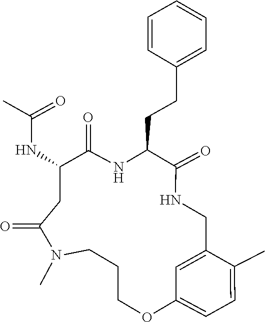 [SMILES: CC(=O)N[C@H]1CC(=O)N(C)CCCOc2ccc(C)c(c2)CNC(=O)[C@H](CCc2ccccc2)NC1=O] | N-((9S,12S)-14,6-dimethyl- 7,10,13-trioxo-12-phenethyl-2- oxa-6, 11,14-triaza-1(1,3)- benzenacyclopentadecaphane- 9-yl)acetamide | 508.3 C₂₈H₃₆N₄O₅ | 509.4 |
| 257 | 247 | 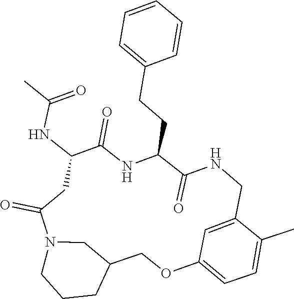 [SMILES: CC(=O)N[C@H]1CC(=O)N2CCCC(COc3ccc(C)c(c3)CNC(=O)[C@H](CCc3ccccc3)NC1=O)C2] | N-((8S,11S)-4⁴-methyl- 7,10,13-trioxo-8-phenethyl-3- oxa-6,9-diaza-1(3,1)- piperidina-4(1,3)- benzenacyclotridecaphane-11- yl)acetamide | 534.3 C₃₀H₃₈N₄O₅ | 535.4 |
| 258 | 248 | 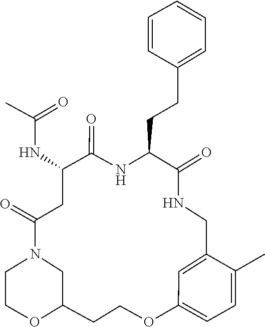 [SMILES: CC(=O)N[C@H]1CC(=O)N2CCOC(CCOc3ccc(C)c(c3)CNC(=O)[C@H](CCc3ccccc3)NC1=O)C2] | N-((9S,12S)-5⁴-methyl- 8,11,14-trioxo-9-phenethyl-4- oxa-7,10-diaza-1(2,4)- morpholina-5(1,3)- benzenacyclotetradecaphane- 12-yl)acetamide | 550.3 C₃₀H₃₈N₄O₆ | 551.4 |
| 259 | 249 | 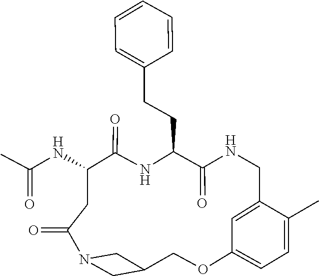 [SMILES: CC(=O)N[C@H]1CC(=O)N2CC(COc3ccc(C)c(c3)CNC(=O)[C@H](CCc3ccccc3)NC1=O)C2] | N-((8S,11S)-4⁴-methyl- 7,10,13-trioxo-8-phenethyl-3- oxa-6,9-diaza-1(3,1)-azetidina- 4(1,3)- benzenacyclotridecaphane-11- yl)acetamide | 506.3 C₂₈H₃₄N₄O₅ | 507.4 |
| 260 | 250 | 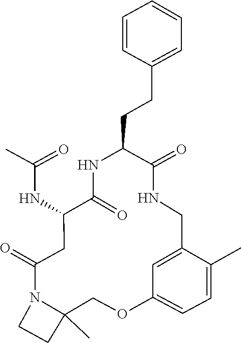 [SMILES: CC(=O)N[C@H]1CC(=O)N2CCC2(C)COc2ccc(C)c(c2)CNC(=O)[C@H](CCc2ccccc2)NC1=O] | N-((8S,11S)-1²,4⁴-dimethyl- 7,10,13-trioxo-8-phenethyl-3- oxa-6,9-diaza-1(2,1)-azetidina- 4(1,3)- benzenacyclotridecaphane-11- yl)acetamide | 520.3 C₂₉H₃₆N₄O₅ | 521.4 |
| 261 | 251 | 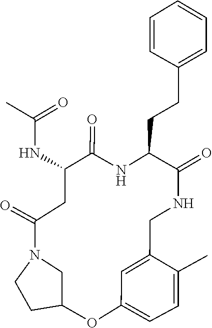 [SMILES: CC(=O)N[C@H]1CC(=O)N2CCC(C2)Oc2ccc(C)c(c2)CNC(=O)[C@H](CCc2ccccc2)NC1=O] | N-((7S,10S)-3⁴-methyl-6,9,12- trioxo-7-phenethyl-2-oxa-5,8- diaza-1(3,1)-pyrrolidina-3(1,3)- benzenacyclododecaphane- 10-yl)acetamide | 506.3 C₂₈H₃₄N₄O₅ | 507.4 |
| 296 | 377 | 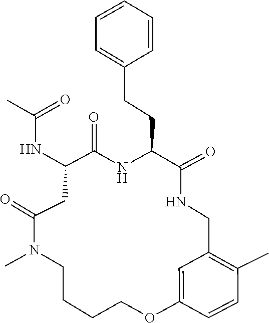 | N-(1⁴,7-dimethyl-8,11,14- trioxo-13-phenethyl-2-oxa- 7,12,15-triaza-1(1,3)- benzenacyclohexadecaphane- 10-yl)acetamide | 522.3 C₂₉H₃₈N₄O₅ | 523.4 |
| 298 | 379 | 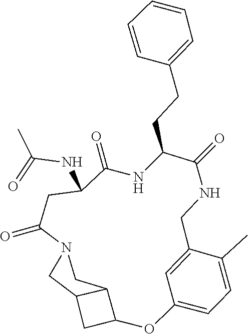 [SMILES: CC(=O)N[C@H]1CC(=O)N2CC3CC(Oc4ccc(C)c(c4)CNC(=O)[C@H](CCc4ccccc4)NC1=O)C3C2] | N-(34-methyl-6,9, 12-trioxo-7- phenethyl-2-oxa-13,5,8-triaza- 1(6,3)-bicyclo[3.2.0]heptana- 3(1,3)- benzenacyclododecaphane- 10-yl)acetamide | 532.3 C₃₀H₃₆N₄O₅ | 533.3 |

### Example A-29: Preparation of Macrocyclic Compounds 265, 282 and 299

### tert-butyl 2-((3-(((S)-2-((S)-2-acetamido-5-(tert-butoxy)-5-oxopentanamido)-4-phenylbutanamido)methyl)-4-methylphenoxy)methyl)-2-methylazetidine-1-carboxylate

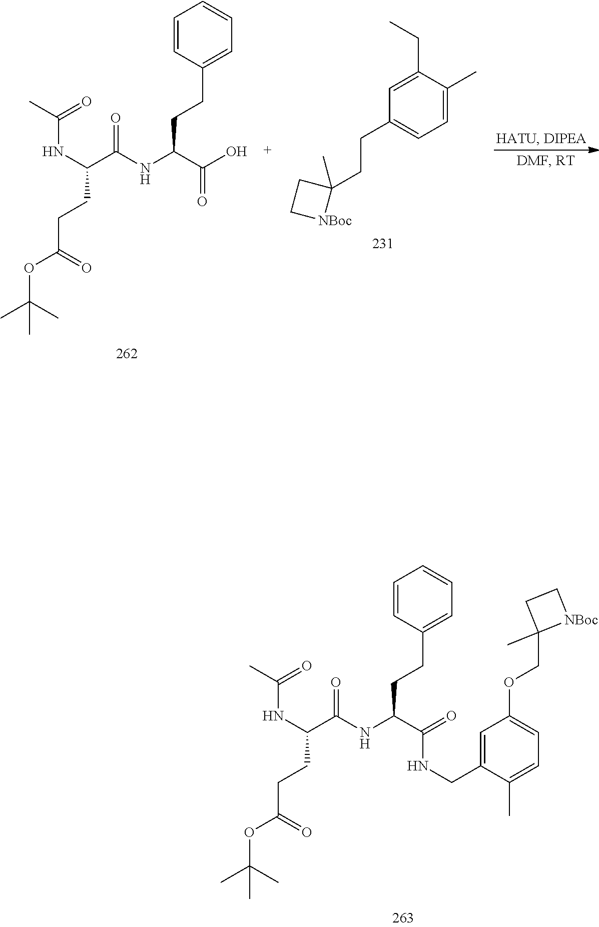 [SMILES: CC(=O)N[C@@H](CCC(=O)OC(C)(C)C)C(=O)N[C@@H](CCc1ccccc1)C(=O)O ; CCc1cc(CCC2(C)CCN2C(=O)OC(C)(C)C)ccc1C ; CC(=O)N[C@@H](CCC(=O)OC(C)(C)C)C(=O)N[C@@H](CCc1ccccc1)C(=O)NCc1cc(OCC2(C)CCN2C(=O)OC(C)(C)C)ccc1C]

Amide couplings between benzylamine derivatives and dipeptide 2 were accomplished according to general procedure B, typically using 100 mg dipeptide 262. As an example compound 263 was synthesized from 112 mg dipeptide 262, 115 mg amine 231, 125 mg HATU and 214 μl DIPEA in DMF at room temperature. Purification was achieved by normal phase column chromatography (silica, cyclohexane/ethyl acetate gradient). Additional examples are disclosed in the following table.

TABLE A-29

| Cpd | From | | | Exactm | [M + H]⁺ |
|---|---|---|---|---|---|
| no | amine | structure | name | mass. | found |
| 263 | 231 | 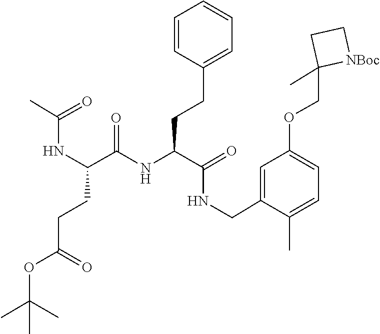 [SMILES: CC(=O)N[C@@H](CCC(=O)OC(C)(C)C)C(=O)N[C@@H](CCc1ccccc1)C(=O)NCc1cc(OCC2(C)CCN2C(=O)OC(C)(C)C)ccc1C] | tert-butyl 2-((3-(((S)-2-((S)- 2-acetamido-5-(tert- butoxy)-5- oxopentanamido)-4- phenylbutanamido)methyl)- 4-methylphenoxy)methyl)- 2-methylazetidine-1- carboxylate | 708.4 | 709.7 |
| 274 | 270 | 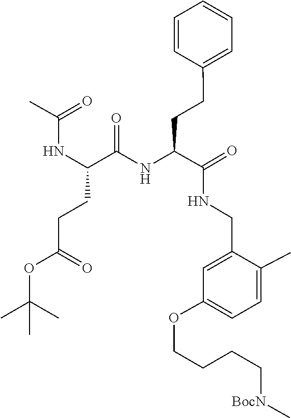 [SMILES: CC(=O)N[C@@H](CCC(=O)OC(C)(C)C)C(=O)N[C@@H](CCc1ccccc1)C(=O)NCc1cc(OCCCCN(C)C(=O)OC(C)(C)C)ccc1C] | (S)-tert-butyl 4-acetamido- 5-(((S)-1-((5-(4-((tert- butoxycarbonyl)(methyl) amino)butoxy)-2- methylbenzyl)amino)-1- oxo-4-phenylbutan-2- yl)amino)-5- oxopentanoate | 710.4 | 711.5 |
| 380 | 188 | 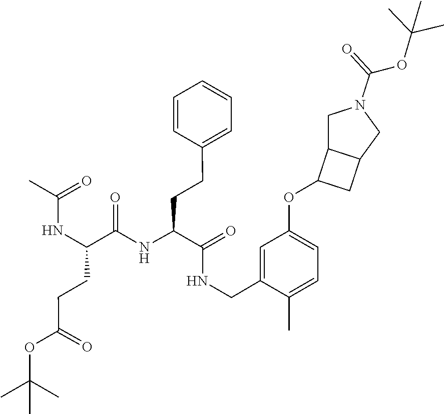 [SMILES: CC(=O)N[C@@H](CCC(=O)OC(C)(C)C)C(=O)N[C@@H](CCc1ccccc1)C(=O)NCc1cc(OC2CC3CN(C(=O)OC(C)(C)C)CC32)ccc1C] | tert-butyl 6-(3-(((S)-2-((S)- 2-acetamido-5-(tert- butoxy)-5- oxopentanamido)-4- phenylbutanamido)methyl)- 4-methylphenoxy)-3- azabicyclo[3.2.0]heptane- 3-carboxylate | 720.4 | 721.5 |

### (4S)-4-acetamido-5-(((2S)-1-((2-methyl-5-((2-methylazetidin-2-yl)methoxy)benzyl)amino)-1-oxo-4-phenylbutan-2-yl)amino)-5-oxopentanoic acid (264)

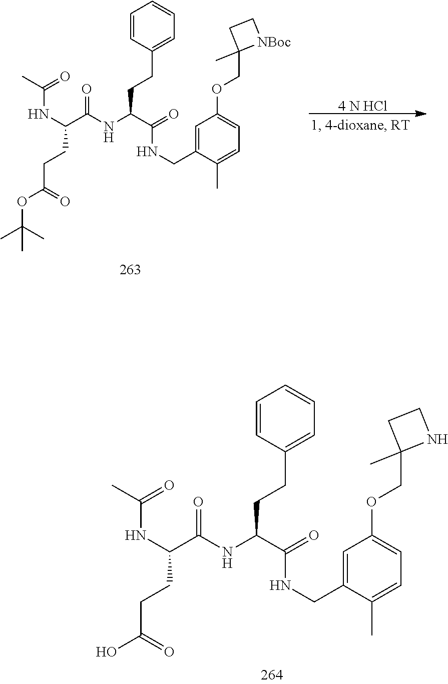 [SMILES: CC(=O)N[C@@H](CCC(=O)OC(C)(C)C)C(=O)N[C@@H](CCc1ccccc1)C(=O)NCc1cc(OCC2(C)CCN2C(=O)OC(C)(C)C)ccc1C ; "4 N HCl" ; CC(=O)N[C@@H](CCC(=O)O)C(=O)N[C@@H](CCc1ccccc1)C(=O)NCc1cc(OCC2(C)CCN2)ccc1C]

Protecting groups were cleaved from 117 mg compound 263 according to general procedure D stirring compounds in 10 ml 4 N HCl/1,4-dioxane at room temperature. After deprotection was complete solvent was removed and the crude dissolved in acetonitrile and concentrated twice. Product used without further purification.

As exemplified by compound 264 additional examples disclosed in the following table were prepared.

TABLE A-30

| Cpd | From | | | Exact | [M + H]⁺ |
|---|---|---|---|---|---|
| no | cpd | structure | name | mass | found |
| 264 | 263 | 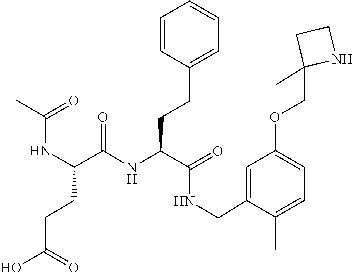 [SMILES: CC(=O)N[C@@H](CCC(=O)O)C(=O)N[C@@H](CCc1ccccc1)C(=O)NCc1cc(OCC2(C)CCN2)ccc1C] | (4S)-4-acetamido-5-(((2S)- 1-((2-methyl-5-((2- methylazetidin-2- yl)methoxy)benzyl)amino)- 1-oxo-4-phenylbutan-2- yl)amino)-5-oxopentanoic acid | 552.3 | 553.4 |
| 278 | 274 | 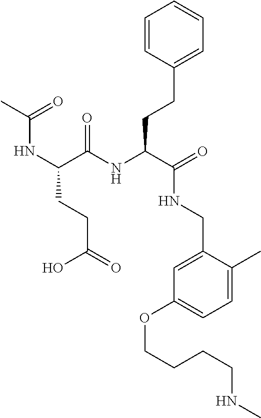 [SMILES: CNCCCCOc1ccc(C)c(CNC(=O)[C@H](CCc2ccccc2)NC(=O)[C@H](CCC(=O)O)NC(C)=O)c1] | (S)-4-acetamido-5-(((S)-1- ((2-methyl-5-(4- (methylamino)butoxy) benzyl)amino)-1-oxo-4- phenylbutan-2-yl)amino)- 5-oxopentanoic acid | 554.3 | 555.4 |
| 381 | 380 | 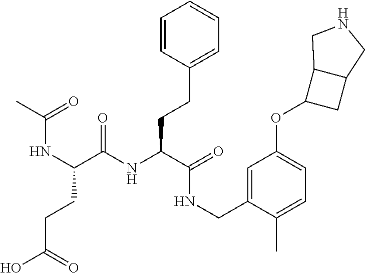 [SMILES: CC(=O)N[C@@H](CCC(=O)O)C(=O)N[C@@H](CCc1ccccc1)C(=O)NCc1cc(OC2CC3CNCC32)ccc1C] | (4S)-5-(((2S)-1-((5-(3- azabicyclo[3.2.0]heptan-6- yloxy)-2- methylbenzyl)amino)-1- oxo-4-phenylbutan-2- yl)amino)-4-acetamido-5- oxopentanoic acid | 564.3 | 565.4 |

### N-((8S,11S)-1²,4⁴-dimethyl-7,10,14-trioxo-8-phenethyl-3-oxa-6,9-diaza-1(2,1)-azetidina-4(1,3)-benzenacyclotetradecaphane-11-yl)acetamide (265)

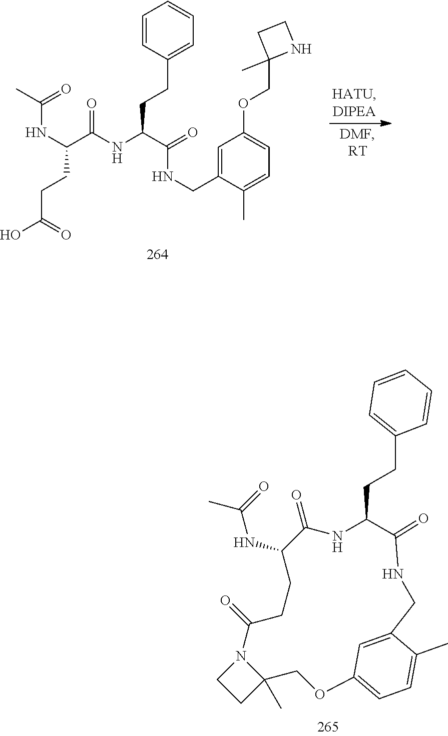 [SMILES: CC(=O)N[C@@H](CCC(=O)O)C(=O)N[C@@H](CCc1ccccc1)C(=O)NCc1cc(OCC2(C)CCN2)ccc1C ; CC(=O)N[C@H]1CCC(=O)N2CCC2(C)COc2ccc(C)c(c2)CNC(=O)[C@H](CCc2ccccc2)NC1=O]

Macrocyclization was achieved according to general procedure A with 0.165 mmol amino acid 264. Reaction mixtures were diluted with some methanol and purified via HPLC. As exemplified by compound 265 additional examples disclosed in the following table were prepared.

TABLE A-31

| Cpd | From | | | Exact | [M + H]⁺ |
|---|---|---|---|---|---|
| no | cpd | structure | name | mass | found |
| 265 | 264 | 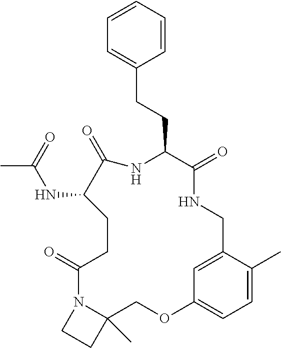 [SMILES: CC(=O)N[C@H]1CCC(=O)N2CCC2(C)COc2ccc(C)c(c2)CNC(=O)[C@H](CCc2ccccc2)NC1=O] | N-((8S,11S)-1²,4⁴-dimethyl- 7,10,14-trioxo-8-phenethyl-3- oxa-6,9-diaza-1(2,1)- azetidina-4(1,3)- benzenacyclotetradecaphane- 11-yl)acetamide | 534.3 C₃₀H₃₈ N₄O₅ | 535.4 |
| 282 | 278 | 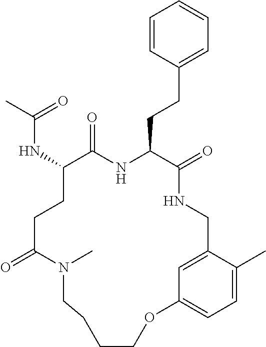 [SMILES: CC(=O)N[C@H]1CCC(=O)N(C)CCCCOc2ccc(C)c(c2)CNC(=O)[C@H](CCc2ccccc2)NC1=O] | N-((11S,14S)-1⁴,7-dimethyl- 8,12,15-trioxo-14-phenethyl- 2-oxa-7,13,16-triaza-1(1,3)- benzenacycloheptadecaphane- 11-yl)acetamide | 536.3 C₃₀H₄₀ N₄O₅ | 537.3 |
| 299 | 381 | 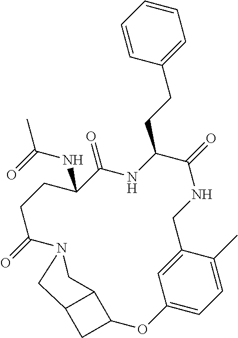 [SMILES: CC(=O)N[C@H]1CCC(=O)N2CC3CC(Oc4ccc(C)c(c4)CNC(=O)[C@H](CCc4ccccc4)NC1=O)C3C2] | N-(3⁴-methyl-6,9,13-trioxo-7- phenethyl-2-oxa-13,5,8- triaza-1(6,3)- bicyclo[3.2.0]heptana-3(1,3)- benzenacyclotridecaphane- 10-yl)acetamide | 546.3 C₃₁H₃₈ N₄O₅ | 547.3 |

### Example A-30: Preparation of Macrocyclic Compounds 269, 280, 281 and 29

### (S)-tert-butyl 5-acetamido-6-(((S)-1-((5-(4-((tert-butoxycarbonyl)amino)butoxy)-2-methylbenzyl)amino)-1-oxo-4-phenylbutan-2-yl)amino)-6-oxohexanoate (267)

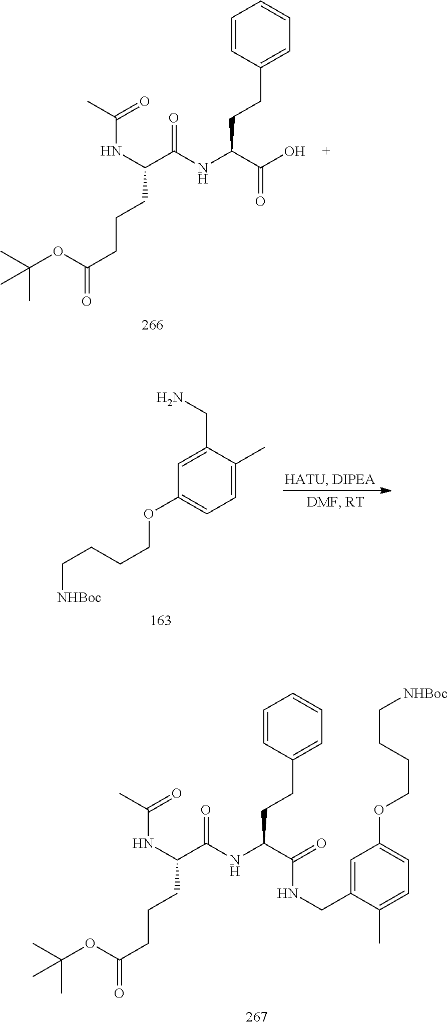 [SMILES: CC(=O)N[C@@H](CCCC(=O)OC(C)(C)C)C(=O)N[C@@H](CCc1ccccc1)C(=O)O ; Cc1ccc(OCCCCNC(=O)OC(C)(C)C)cc1CN ; CC(=O)N[C@@H](CCCC(=O)OC(C)(C)C)C(=O)N[C@@H](CCc1ccccc1)C(=O)NCc1cc(OCCCCNC(=O)OC(C)(C)C)ccc1C]

Amide couplings between benzylamine derivatives and dipeptide 266 were accomplished according to general procedure B, typically using 100 mg dipeptide 262. As an example compound 263 was synthesized from 113 mg dipeptide 266, 107 mg amine 163, 122 mg HATU and 280 μl DIPEA in DMF at room temperature. Purification was achieved by normal phase column chromatography (silica, cyclohexane/ethyl acetate gradient). Additional examples are disclosed in the following table.

TABLE A-32

| Cpd | From | | | Exact | [M + H]⁺ |
|---|---|---|---|---|---|
| no | amine | structure | name | mass | found |
| 267 | 163 | 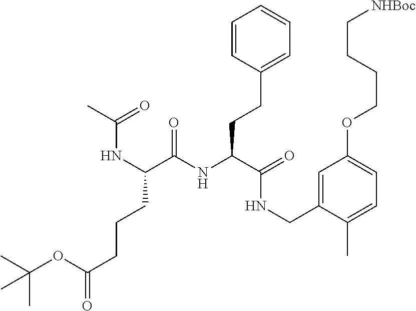 [SMILES: CC(=O)N[C@@H](CCCC(=O)OC(C)(C)C)C(=O)N[C@@H](CCc1ccccc1)C(=O)NCc1cc(OCCCCNC(=O)OC(C)(C)C)ccc1C] | (S)-tert-butyl 5-acetamido- 6-(((S)-1-((5-(4-((tert- butoxycarbonyl)amino)buto xy)-2-methylbenzyl)amino)- 1-oxo-4-phenylbutan-2- yl)amino)-6-oxohexanoate | 710.4 | 710.7 |
| 272 | 227 | 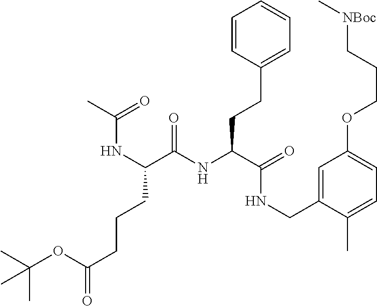 [SMILES: CC(=O)N[C@@H](CCCC(=O)OC(C)(C)C)C(=O)N[C@@H](CCc1ccccc1)C(=O)NCc1cc(OCCCN(C)C(=O)OC(C)(C)C)ccc1C] | (S)-tert-butyl 5-acetamido- 6-(((S)-1-((5-(3-((tert- butoxycarbonyl)(methyl)ami no)propoxy)-2- methylbenzyl)amino)-1-oxo- 4-phenylbutan-2-yl)amino)- 6-oxohexanoate | 722.4 | 723.8 |
| 273 | 232 | 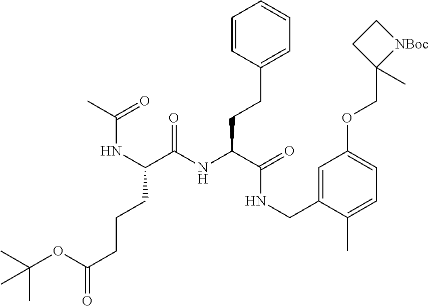 [SMILES: CC(=O)N[C@@H](CCCC(=O)OC(C)(C)C)C(=O)N[C@@H](CCc1ccccc1)C(=O)NCc1cc(OCC2(C)CCN2C(=O)OC(C)(C)C)ccc1C] | tert-butyl 2-((3-(((S)-2-((S)- 2-acetamido-6-(tert-butoxy)- 6-oxohexanamido)-4- phenylbutanamido)methyl)- 4-methylphenoxy)methyl)-2- methylazetidine-1- carboxylate | 710.4 | 711.5 |
| 374 | 270 | 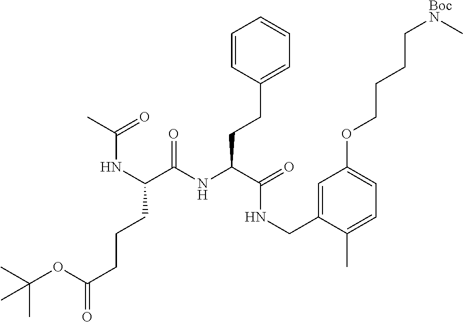 [SMILES: CC(=O)N[C@@H](CCCC(=O)OC(C)(C)C)C(=O)N[C@@H](CCc1ccccc1)C(=O)NCc1cc(OCCCCN(C)C(=O)OC(C)(C)C)ccc1C] | 6-(((S)-1 -((5-(4-((tert- butoxycarbonyl)(methyl) amino)butoxy)-2- methylbenzyl)amino)-1-oxo- 4-phenylbutan-2-yl)amino)- 6-oxohexanoate | 724.4 | 725.6 |

### (S)-5-acetamido-6-(((S)-1-((5-(4-aminobutoxy)-2-methylbenzyl)amino)-1-oxo-4-phenylbutan-2-yl)amino)-6-oxohexanoic acid (268)

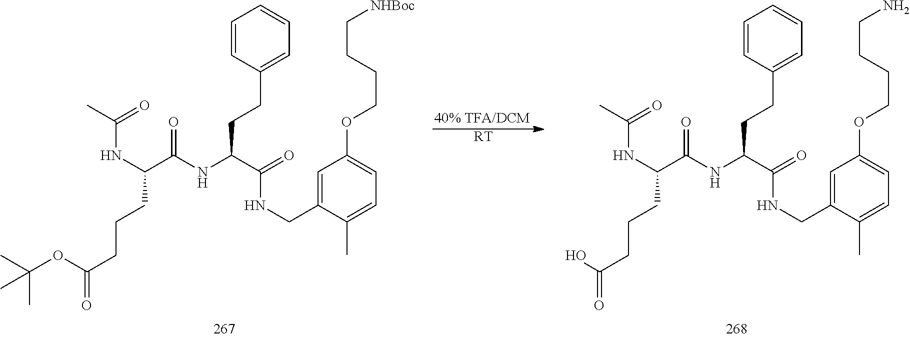 [SMILES: CC(=O)N[C@@H](CCCC(=O)OC(C)(C)C)C(=O)N[C@@H](CCc1ccccc1)C(=O)NCc1cc(OCCCCNC(=O)OC(C)(C)C)ccc1C ; "RT" ; CC(=O)N[C@@H](CCCC(=O)O)C(=O)N[C@@H](CCc1ccccc1)C(=O)NCc1cc(OCCCCN)ccc1C]

Protecting groups were cleaved from 78 mg compound 267 according to general procedure D stirring compounds in 5 ml 40% TFA/DCM at room temperature. After deprotection was complete solvent was removed and the crude dissolved in acetonitrile and concentrated twice. Product used without further purification.

As exemplified by compound 268 additional examples disclosed in the following table were prepared.

TABLE A-33

| Cpd | From | | | Exact | [M + H]⁺ |
|---|---|---|---|---|---|
| no | cpd | structure | name | mass | found |
| 268 | 267 | 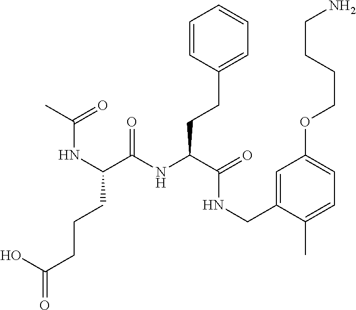 [SMILES: CC(=O)N[C@@H](CCCC(=O)O)C(=O)N[C@@H](CCc1ccccc1)C(=O)NCc1cc(OCCCCN)ccc1C] | (S)-5-acetamido-6-(((S)-1- ((5-(4-aminobutoxy)-2- methylbenzyl)amino)-1-oxo- 4-phenylbutan-2-yl)amino)- 6-oxohexanoic acid | 554.3 | 555.4 |
| 276 | 272 | 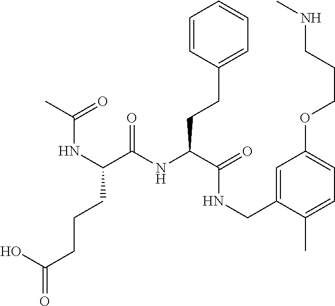 [SMILES: CNCCCOc1ccc(C)c(CNC(=O)[C@H](CCc2ccccc2)NC(=O)[C@H](CCCC(=O)O)NC(C)=O)c1] | (S)-5-acetamido-6-(((S)-1- ((2-methyl-5-(3- (methylamino)propoxy) benzyl)amino)-1-oxo-4- phenylbutan-2-yl)amino)-6- oxohexanoic acid | 554.3 | 555.4 |
| 277 | 273 | 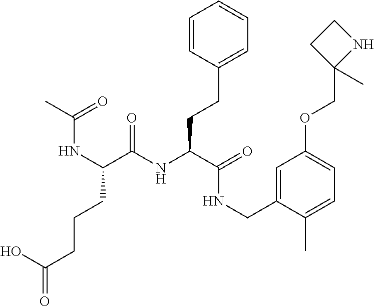 [SMILES: CC(=O)N[C@@H](CCCC(=O)O)C(=O)N[C@@H](CCc1ccccc1)C(=O)NCc1cc(OCC2(C)CCN2)ccc1C] | (5S)-5-acetamido-6-(((2S)- 1-((2-methyl-5-((2- methylazetidin-2- yl)methoxy)benzyl)amino)- 1-oxo-4-phenylbutan-2- yl)amino)-6-oxohexanoic acid | 566.3 | 567.4 |
| 375 | 374 | 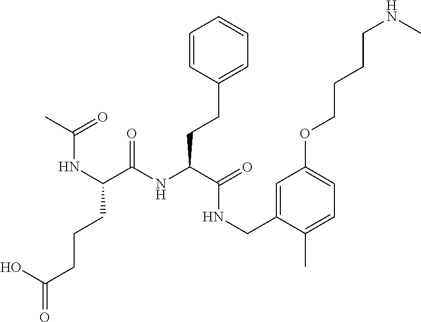 [SMILES: CNCCCCOc1ccc(C)c(CNC(=O)[C@H](CCc2ccccc2)NC(=O)[C@H](CCCC(=O)O)NC(C)=O)c1] | (S)-5-acetamido-6-(((S)-1- ((2-methyl-5-(4- (methylamino)butoxy)benzyl) amino)-1-oxo-4- phenylbutan-2-yl)amino)-6- oxohexanoic acid | 568.3 | 569.4 |

### N-((12S,15S)-1⁴-methyl-8,13,16-trioxo-15-phenethyl-2-oxa-7,14,17-triaza-1(1,3)-benzenacyclooctadecaphane-12-yl)acetamide (269)

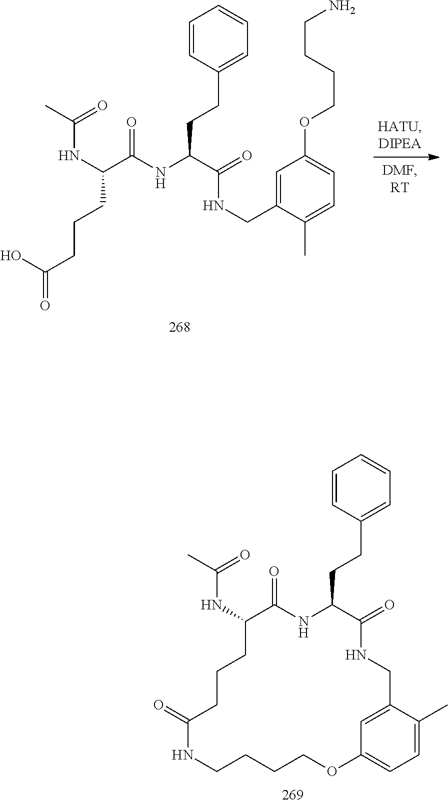 [SMILES: CC(=O)N[C@@H](CCCC(=O)O)C(=O)N[C@@H](CCc1ccccc1)C(=O)NCc1cc(OCCCCN)ccc1C ; CC(=O)N[C@H]1CCCC(=O)NCCCCOc2ccc(C)c(c2)CNC(=O)[C@H](CCc2ccccc2)NC1=O]

Macrocyclization was achieved according to general procedure A with 0.120 mmol amino acid 268. Reaction mixtures were diluted with some methanol and purified via HPLC. As exemplified by compound 269 additional examples disclosed in the following table were prepared.

TABLE A-34

| Cpd | From | | | [M + H]⁺ | [M + H]⁺ |
|---|---|---|---|---|---|
| no | cpd | structure | name | calc. | found |
| 269 | 268 | 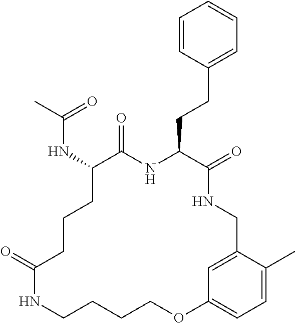 [SMILES: CC(=O)N[C@H]1CCCC(=O)NCCCCOc2ccc(C)c(c2)CNC(=O)[C@H](CCc2ccccc2)NC1=O] | N-((12S,15S)-1⁴-methyl- 8,13,16-trioxo-15- phenethyl-2-oxa-7,14,17- triaza-1(1,3)- benzenacyclooctadecaphane- 12-yl)acetamide | 536.3 C₃₀H₄₀ N₄O₅ | 537.4 |
| 280 | 276 | 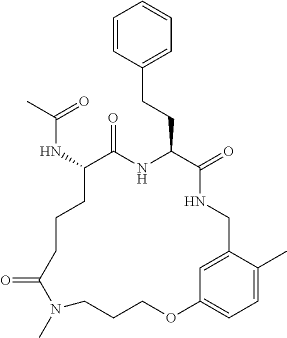 [SMILES: CC(=O)N[C@H]1CCCC(=O)N(C)CCCOc2ccc(C)c(c2)CNC(=O)[C@H](CCc2ccccc2)NC1=O] | N-((11S,14S)-1⁴,6-dimethyl- 7,12,15-trioxo-14- phenethyl-2-oxa-6,13,16- triaza-1(1,3)- benzenacycloheptadecaphane- 11-yl)acetamide | 536.3 C₃₀H₄₀ N₄O₅ | 537.4 |
| 281 | 277 | 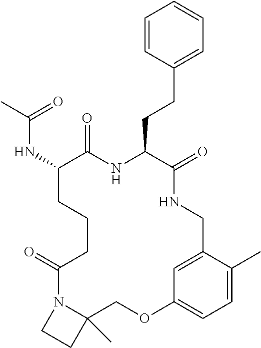 [SMILES: CC(=O)N[C@H]1CCCC(=O)N2CCC2(C)COc2ccc(C)c(c2)CNC(=O)[C@H](CCc2ccccc2)NC1=O] | N-((8S,11S)-1²,4⁴-dimethyl- 7,10,15-trioxo-8-phenethyl- 3-oxa-6,9-diaza-1(2,1)- azetidina-4(1,3)- benzenacyclopentadecaphane- 11-yl)acetamide | 548.3 C₃₁H₄₂ N₄O₆ | 549.5 |
| 295 | 375 | 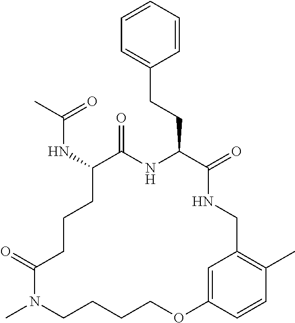 [SMILES: CC(=O)N[C@H]1CCCC(=O)N(C)CCCCOc2ccc(C)c(c2)CNC(=O)[C@H](CCc2ccccc2)NC1=O] | N-(1⁴,7-dimethyl-8,13,16- trioxo-15-phenethyl-2-oxa- 7,14,17-triaza-1(1,3)- benzenacyclooctadecaphane- 12-yl)acetamide | 550.3 C₃₁H₄₂ N₄O₅ | 551.4 |

### Example A-31: Preparation of Macrocyclic Compounds 336, and 549-564

### tert-butyl 3-(2-(3-((5S)-5-(2-(tert-butoxy)-2-oxoethyl)-3,6,9-trioxo-1-phenyl-8-(2-(pyridin-4-yl)ethyl)-2-oxa-4,7,10-triazaundecan-11-yl)-4-methylphenoxy)ethyl)piperidine-1-carboxylate (435)

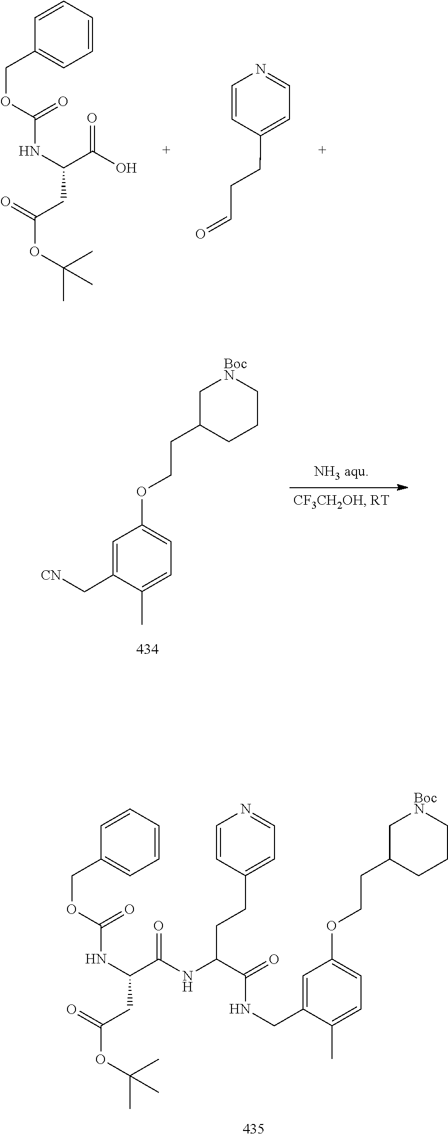

For this Ugi reaction with 400 mg 3-(pyridin-4-yl)propanal and 960 mg Cbz-Asp(OtBu)-OH were dissolved in 3.5 ml 2,2,2-trifluoroethanol. 700 μl ammonia solution (30%, aqueous) and 715 mg isonitrile 434 were added and the mixture was stirred at room temperature overnight. Additional ammonia solution (350 μl) and 250 mg aldehyde in 3.5 ml 2,2,2-trifluoroethanol were added and stirring at room temperature was continued for another day. Saturated NaHCO₃ solution was added and the mixture was extracted with ethyl acetate. After drying the combined organic phases over MgSO₄ and removing volatiles under reduced pressure the product was obtained after normal phase column chromatography (silica, cyclohexane/ethyl acetate gradient).

Formula: C₄₅H₆₁N₅O₉, exact mass: 815.4, found: 716.4 [M+H]⁺

The examples in the following table were prepared according to the procedure described for compound 435 but utilizing Ac-Asp(OtBu)-OH instead of Cbz-Asp(OtBu)-OH and related aldehydes:

TABLE A-35

| Cpd | | | Exact | [M + H]⁺ |
|---|---|---|---|---|
| no | structure | name | mass | found |
| 541 | 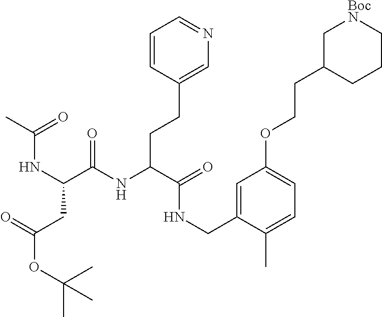 [SMILES: CC(=O)N[C@@H](CC(=O)OC(C)(C)C)C(=O)NC(CCc1cccnc1)C(=O)NCc1cc(OCCC2CCCN(C(=O)OC(C)(C)C)C2)ccc1C] | tert-butyl 3-(2-(3-((2-((S)-2-acetamido- 4-(tert-butoxy)-4-oxobutanamido)-4- (pyridin-3-yl)butanamido)methyl)-4- methylphenoxy)ethyl)piperidine-1- carboxylate | 723.4 | 724.8 |
| 542 | 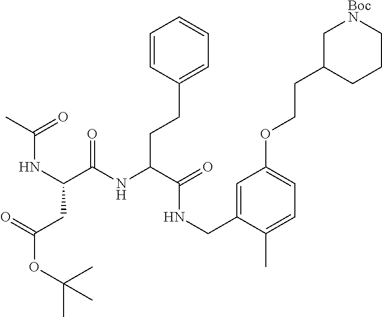 [SMILES: CC(=O)N[C@@H](CC(=O)OC(C)(C)C)C(=O)NC(CCc1ccccc1)C(=O)NCc1cc(OCCC2CCCN(C(=O)OC(C)(C)C)C2)ccc1C] | tert-butyl 3-(2-(3-((2-((S)-2-acetamido- 4-(tert-butoxy)-4-oxobutanamido)-4- phenylbutanamido)methyl)-4- methylphenoxy)ethyl)piperidine-1- carboxylate | 722.4 | 723.7 |
| 543 | 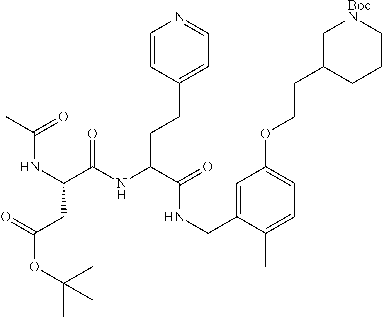 [SMILES: CC(=O)N[C@@H](CC(=O)OC(C)(C)C)C(=O)NC(CCc1ccncc1)C(=O)NCc1cc(OCCC2CCCN(C(=O)OC(C)(C)C)C2)ccc1C] | tert-butyl 3-(2-(3-((2-((S)-2-acetamido- 4-(tert-butoxy)-4-oxobutanamido)-4- (pyridin-4-yl)butanamido)methyl)-4- methylphenoxy)ethyl)piperidine-1- carboxylate | 723.4 | 724.5 |
| 544 | 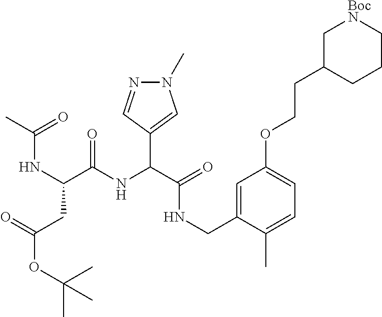 [SMILES: CC(=O)N[C@@H](CC(=O)OC(C)(C)C)C(=O)NC(C(=O)NCc1cc(OCCC2CCCN(C(=O)OC(C)(C)C)C2)ccc1C)c1cnn(C)c1] | tert-butyl 3-(2-(3-((2-((S)-2-acetamido- 4-(tert-butoxy)-4-oxobutanamido)-2- (1-methyl-1H-pyrazol-4- yl)acetamido)methyl)-4- methylphenoxy)ethyl)piperidine-1- carboxylate | 698.4 | 699.6 |
| 517 | 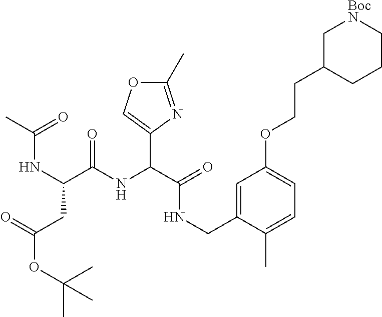 [SMILES: CC(=O)N[C@@H](CC(=O)OC(C)(C)C)C(=O)NC(C(=O)NCc1cc(OCCC2CCCN(C(=O)OC(C)(C)C)C2)ccc1C)c1coc(C)n1] | tert-butyl 3-(2-(3-((2-((S)-2-acetamido- 4-(tert-butoxy)-4-oxobutanamido)-2- (2-methyloxazol-4- yl)acetamido)methyl)-4- methylphenoxy)ethyl)piperidine-1- carboxylate | 699.4 | 700.6 |
| 518 | 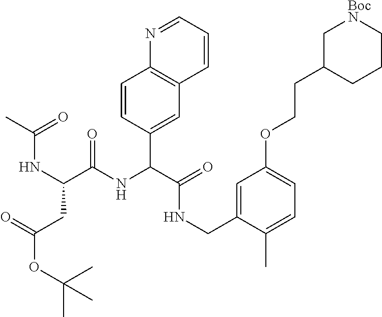 [SMILES: CC(=O)N[C@@H](CC(=O)OC(C)(C)C)C(=O)NC(C(=O)NCc1cc(OCCC2CCCN(C(=O)OC(C)(C)C)C2)ccc1C)c1ccc2ncccc2c1] | tert-butyl 3-(2-(3-((2-((S)-2-acetamido- 4-(tert-butoxy)-4-oxobutanamido)-2- (quinolin-6-yl)acetamido)methyl)-4- methylphenoxy)ethyl)piperidine-1- carboxylate | 745.4 | 746.7 |
| 519 | 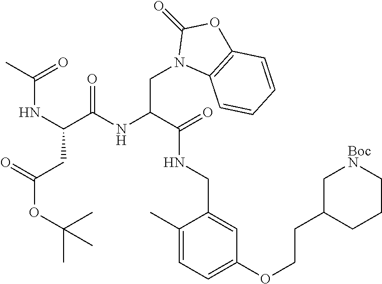 [SMILES: CC(=O)N[C@@H](CC(=O)OC(C)(C)C)C(=O)NC(Cn1c(=O)oc2ccccc21)C(=O)NCc1cc(OCCC2CCCN(C(=O)OC(C)(C)C)C2)ccc1C] | tert-butyl 3-(2-(3-((2-((S)-2-acetamido- 4-(tert-butoxy)-4-oxobutanamido)-3- (2-oxobenzo[d]oxazol-3(2H)- yl)propanamido)methyl)-4- methylphenoxy)ethyl)piperidine-1- carboxylate | 765.4 | 766.7 |
| 520 | 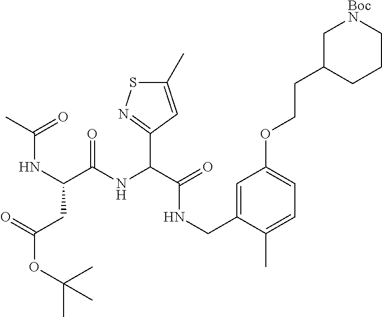 [SMILES: CC(=O)N[C@@H](CC(=O)OC(C)(C)C)C(=O)NC(C(=O)NCc1cc(OCCC2CCCN(C(=O)OC(C)(C)C)C2)ccc1C)c1cc(C)sn1] | tert-butyl 3-(2-(3-((2-((S)-2-acetamido- 4-(tert-butoxy)-4-oxobutanamido)-2- (5-methylisothiazol-3- yl)acetamido)methyl)-4- methylphenoxy)ethyl)piperidine-1- carboxylate | 715.4 | 716.8 |
| 521 | 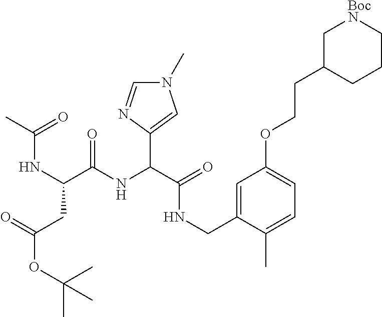 [SMILES: CC(=O)N[C@@H](CC(=O)OC(C)(C)C)C(=O)NC(C(=O)NCc1cc(OCCC2CCCN(C(=O)OC(C)(C)C)C2)ccc1C)c1cn(C)cn1] | tert-butyl 3-(2-(3-((2-((S)-2-acetamido- 4-(tert-butoxy)-4-oxobutanamido)-2- (1-methyl-1H-imidazol-4- yl)acetamido)methyl)-4- methylphenoxy)ethyl)piperidine-1- carboxylate | 698.4 | 699.7 |
| 522 | 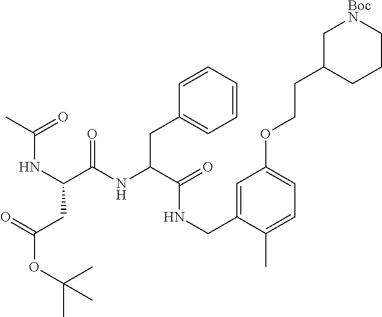 [SMILES: CC(=O)N[C@@H](CC(=O)OC(C)(C)C)C(=O)NC(Cc1ccccc1)C(=O)NCc1cc(OCCC2CCCN(C(=O)OC(C)(C)C)C2)ccc1C] | tert-butyl 3-(2-(3-((2-((S)-2-acetamido- 4-(tert-butoxy)-4-oxobutanamido)-3- phenylpropanamido)methyl)-4- methylphenoxy)ethyl)piperidine-1- carboxylate | 708.4 | 709.8 |
| 523 | 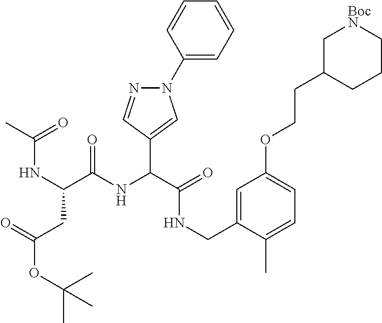 [SMILES: CC(=O)N[C@@H](CC(=O)OC(C)(C)C)C(=O)NC(C(=O)NCc1cc(OCCC2CCCN(C(=O)OC(C)(C)C)C2)ccc1C)c1cnn(-c2ccccc2)c1] | tert-butyl 3-(2-(3-((2-((S)-2-acetamido- 4-(tert-butoxy)-4-oxobutanamido)-2- (1-phenyl-1H-pyrazol-4- yl)acetamido)methyl)-4- methylphenoxy)ethyl)piperidine-1- carboxylate | 760.4 | 761.5 |
| 524 | 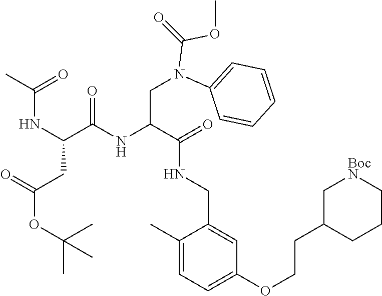 [SMILES: COC(=O)N(CC(NC(=O)[C@H](CC(=O)OC(C)(C)C)NC(C)=O)C(=O)NCc1cc(OCCC2CCCN(C(=O)OC(C)(C)C)C2)ccc1C)c1ccccc1] | tert-butyl 3-(2-(3-((2-((S)-2-acetamido- 4-(tert-butoxy)-4-oxobutanamido)-3- ((methoxycarbonyl)(phenyl)amino) propanamido)methyl)-4- methylphenoxy)ethyl)piperidine-1- carboxylate | 781.4 | 782.8 |
| 525 | 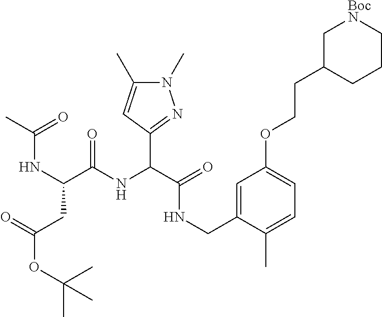 [SMILES: CC(=O)N[C@@H](CC(=O)OC(C)(C)C)C(=O)NC(C(=O)NCc1cc(OCCC2CCCN(C(=O)OC(C)(C)C)C2)ccc1C)c1cc(C)n(C)n1] | tert-butyl 3-(2-(3-((2-((S)-2-acetamido- 4-(tert-butoxy)-4-oxobutanamido)-2- (1,5-dimethyl-1H-pyrazol-3- yl)acetamido)methyl)-4- methylphenoxy)ethyl)piperidine-1- carboxylate | 712.4 | 713.8 |
| 526 | 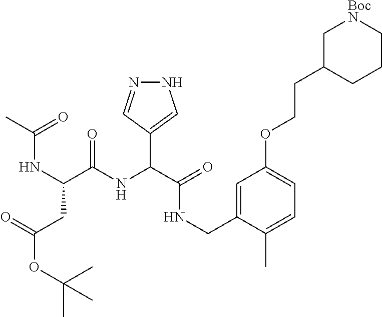 [SMILES: CC(=O)N[C@@H](CC(=O)OC(C)(C)C)C(=O)NC(C(=O)NCc1cc(OCCC2CCCN(C(=O)OC(C)(C)C)C2)ccc1C)c1cn[nH]c1] | tert-butyl 3-(2-(3-((2-((S)-2-acetamido- 4-(tert-butoxy)-4-oxobutanamido)-2- (1H-pyrazol-3-yl)acetamido)methyl)-4- methylphenoxy)ethyl)piperidine-1- carboxylate | 684.4 | 685.7 |
| 527 | 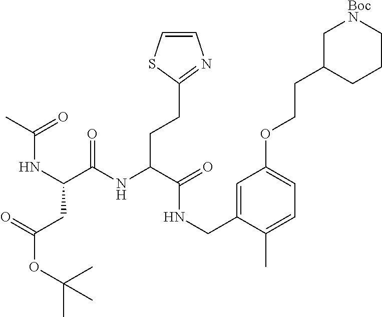 [SMILES: CC(=O)N[C@@H](CC(=O)OC(C)(C)C)C(=O)NC(CCc1nccs1)C(=O)NCc1cc(OCCC2CCCN(C(=O)OC(C)(C)C)C2)ccc1C] | tert-butyl 3-(2-(3-((2-((S)-2-acetamido- 4-(tert-butoxy)-4-oxobutanamido)-4- (thiazol-2-yl)butanamido)methyl)-4- methylphenoxy)ethyl)piperidine-1- carboxylate | 729.4 | 730.7 |
| 528 | 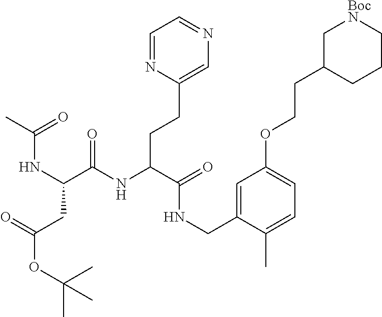 [SMILES: CC(=O)N[C@@H](CC(=O)OC(C)(C)C)C(=O)NC(CCc1cnccn1)C(=O)NCc1cc(OCCC2CCCN(C(=O)OC(C)(C)C)C2)ccc1C] | tert-butyl 3-(2-(3-((2-((S)-2-acetamido- 4-(tert-butoxy)-4-oxobutanamido)-4- (pyrazin-2-yl)butanamido)methyl)-4- methylphenoxy)ethyl)piperidine-1- carboxylate | 724.4 | 725.8 |

### (3S)-3-(((benzyloxy)carbonyl)amino)-4-((1-((2-methyl-5-(2-(piperidin-3-yl)ethoxy)benzyl) amino)-1-oxo-4-(pyridin-4-yl)butan-2-yl)amino)-4-oxobutanoic acid (436)

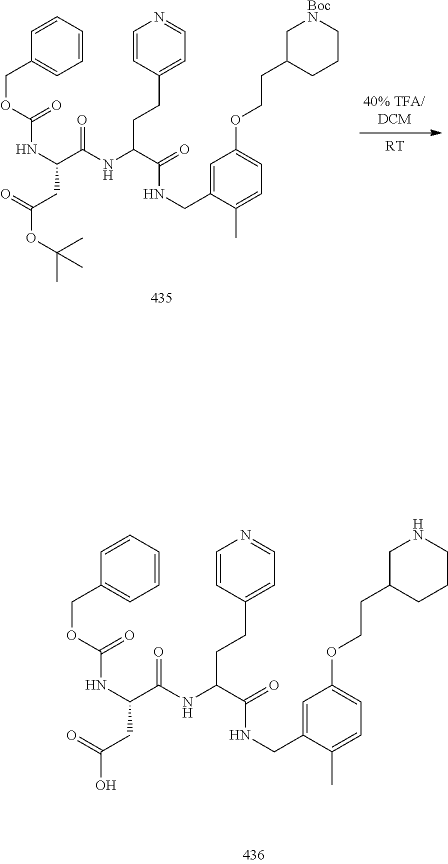 [SMILES: Cc1ccc(OCCC2CCCN(C(=O)OC(C)(C)C)C2)cc1CNC(=O)C(CCc1ccncc1)NC(=O)[C@H](CC(=O)OC(C)(C)C)NC(=O)OCc1ccccc1 ; "RT" ; Cc1ccc(OCCC2CCCNC2)cc1CNC(=O)C(CCc1ccncc1)NC(=O)[C@H](CC(=O)O)NC(=O)OCc1ccccc1]

Protecting groups were cleaved from 750 mg compound 435 according to general procedure D stirring compounds in 5 ml 40% TFA/DCM at room temperature. After deprotection was complete solvent was removed and the crude dissolved in acetonitrile and concentrated twice. Product used without further purification.

Formula: C₃₅H₄₅N₅O₇, exact mass: 659.3, found: 660.3 [M+H]⁺

The examples in the following table were prepared similar to the procedure described for compound 436:

TABLE A-36

| Cpd | From | | | Exact | [M + H]⁺ |
|---|---|---|---|---|---|
| no | Cpd | structure | name | mass | found |
| 545 | 541 | 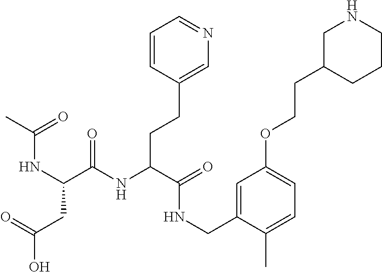 [SMILES: CC(=O)N[C@@H](CC(=O)O)C(=O)NC(CCc1cccnc1)C(=O)NCc1cc(OCCC2CCCNC2)ccc1C] | (3S)-3-acetamido-4-((1-((2- methyl-5-(2-(piperidin-3- yl)ethoxy)benzyl)amino)-1- oxo-4-(pyridin-3-yl)butan-2- yl)amino)-4-oxobutanoic acid | 567.3 | 568.4 |
| 546 | 542 | 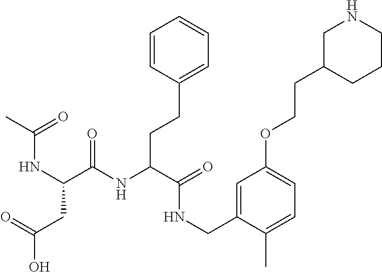 [SMILES: CC(=O)N[C@@H](CC(=O)O)C(=O)NC(CCc1ccccc1)C(=O)NCc1cc(OCCC2CCCNC2)ccc1C] | (3S)-3-acetamido-4-((1-((2- methyl-5-(2-(piperidin-3- yl)ethoxy)benzyl)amino)-1- oxo-4-phenylbutan-2- yl)amino)-4-oxobutanoic acid | 566.3 | 567.4 |
| 547 | 543 | 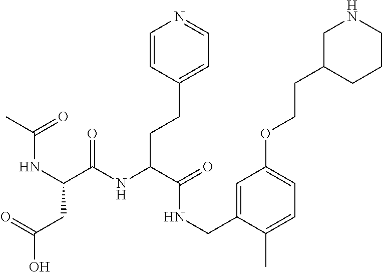 [SMILES: CC(=O)N[C@@H](CC(=O)O)C(=O)NC(CCc1ccncc1)C(=O)NCc1cc(OCCC2CCCNC2)ccc1C] | (3S)-3-acetamido-4-((1-((2- methyl-5-(2-(piperidin-3- yl)ethoxy)benzyl)amino)-1- oxo-4-(pyridin-4-yl)butan-2- yl)amino)-4-oxobutanoic acid | 567.3 | 568.4 |
| 548 | 544 | 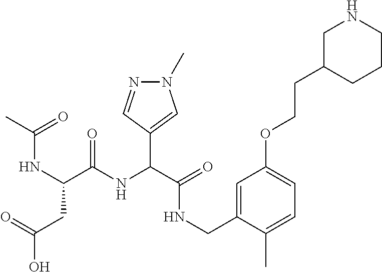 [SMILES: CC(=O)N[C@@H](CC(=O)O)C(=O)NC(C(=O)NCc1cc(OCCC2CCCNC2)ccc1C)c1cnn(C)c1] | (3S)-3-acetamido-4-((1-(1- methyl-1H-pyrazol-4-yl)-2-((2- methyl-5-(2-(piperidin-3- yl)ethoxy)benzyl)amino)-2- oxoethyl)amino)-4- oxobutanoic acid | 542.3 | 543.3 |
| 529 | 517 | 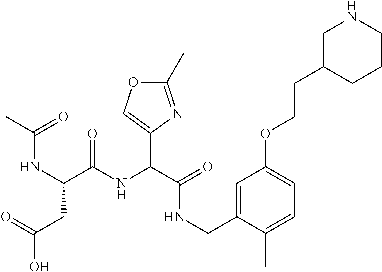 [SMILES: CC(=O)N[C@@H](CC(=O)O)C(=O)NC(C(=O)NCc1cc(OCCC2CCCNC2)ccc1C)c1coc(C)n1] | (3S)-3-acetamido-4-((2-((2- methyl-5-(2-(piperidin-3- yl)ethoxy)benzyl)amino)-1-(2- methyloxazol-4-yl)-2- oxoethyl)amino)-4- oxobutanoic acid | 543.3 | 544.3 |
| 530 | 518 | 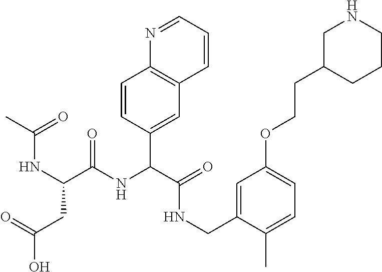 [SMILES: CC(=O)N[C@@H](CC(=O)O)C(=O)NC(C(=O)NCc1cc(OCCC2CCCNC2)ccc1C)c1ccc2ncccc2c1] | (3S)-3-acetamido-4-((2-((2- methyl-5-(2-(piperidin-3- yl)ethoxy)benzyl)amino)-2- oxo-1-(quinolin-6- yl)ethyl)amino)-4-oxobutanoic acid | 589.3 | 590.6 |
| 531 | 519 | 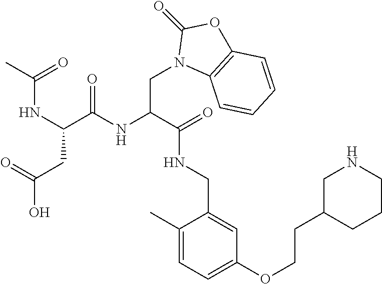 [SMILES: CC(=O)N[C@@H](CC(=O)O)C(=O)NC(Cn1c(=O)oc2ccccc21)C(=O)NCc1cc(OCCC2CCCNC2)ccc1C] | (3S)-3-acetamido-4-((1-((2- methyl-5-(2-(piperidin-3- yl)ethoxy)benzyl)amino)-1- oxo-3-(2-oxobenzo[d]oxazol- 3(2H)-yl)propan-2-yl)amino)- 4-oxobutanoic acid | 609.3 | 610.4 |
| 532 | 520 | 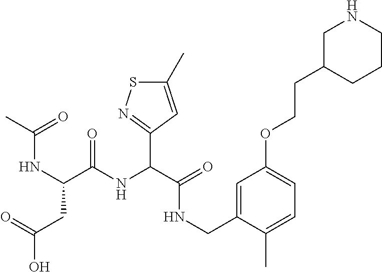 [SMILES: CC(=O)N[C@@H](CC(=O)O)C(=O)NC(C(=O)NCc1cc(OCCC2CCCNC2)ccc1C)c1cc(C)sn1] | (3S)-3-acetamido-4-((2-((2- methyl-5-(2-(piperidin-3- yl)ethoxy)benzyl)amino)-1-(5- methylisothiazol-3-yl)-2- oxoethyl)amino)-4- oxobutanoic acid | 559.2 | 560.4 |
| 533 | 521 | 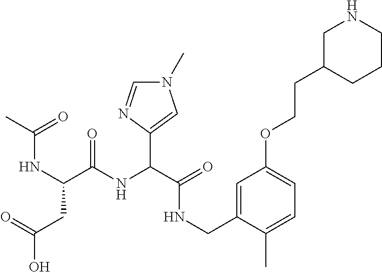 [SMILES: CC(=O)N[C@@H](CC(=O)O)C(=O)NC(C(=O)NCc1cc(OCCC2CCCNC2)ccc1C)c1cn(C)cn1] | (3S)-3-acetamido-4-((1-(1- methyl-1H-imidazol-4-yl)-2- ((2-methyl-5-(2-(piperidin-3- yl)ethoxy)benzyl)amino)-2- oxoethyl)amino)-4- oxobutanoic acid | 542.3 | 543.4 |
| 534 | 522 | 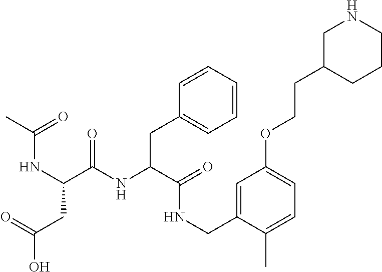 [SMILES: CC(=O)N[C@@H](CC(=O)O)C(=O)NC(Cc1ccccc1)C(=O)NCc1cc(OCCC2CCCNC2)ccc1C] | (3S)-3-acetamido-4-((1-((2- methyl-5-(2-(piperidin-3- yl)ethoxy)benzyl)amino)-1- oxo-3-phenylpropan-2- yl)amino)-4-oxobutanoic acid | 552.3 | 553.4 |
| 535 | 523 | 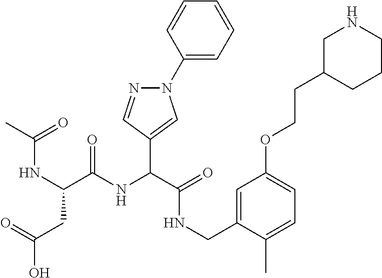 [SMILES: CC(=O)N[C@@H](CC(=O)O)C(=O)NC(C(=O)NCc1cc(OCCC2CCCNC2)ccc1C)c1cnn(-c2ccccc2)c1] | (3S)-3-acetamido-4-((2-((2- methyl-5-(2-(piperidin-3- yl)ethoxy)benzyl)amino)-2- oxo-1-(1-phenyl-1H-pyrazol- 4-yl)ethyl)amino)-4- oxobutanoic acid | 604.3 | 605.6 |
| 536 | 524 | 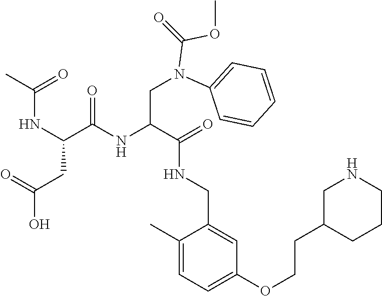 [SMILES: COC(=O)N(CC(NC(=O)[C@H](CC(=O)O)NC(C)=O)C(=O)NCc1cc(OCCC2CCCNC2)ccc1C)c1ccccc1] | (3S)-3-acetamido-4-((3- ((methoxycarbonyl)(phenyl) amino)-1-((2-methyl-5-(2- (piperidin-3- yl)ethoxy)benzyl)amino)-1- oxopropan-2-yl)amino)-4- oxobutanoic acid | 625.3 | 626.6 |
| 537 | 525 | 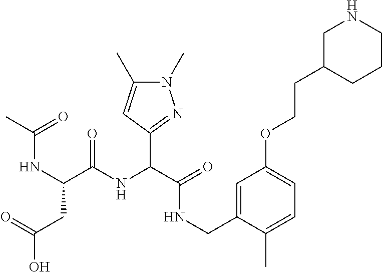 [SMILES: CC(=O)N[C@@H](CC(=O)O)C(=O)NC(C(=O)NCc1cc(OCCC2CCCNC2)ccc1C)c1cc(C)n(C)n1] | (3S)-3-acetamido-4-((1-(1,5- dimethyl-1H-pyrazol-3-yl)-2- ((2-methyl-5-(2-(piperidin-3- yl)ethoxy)benzyl)amino)-2- oxoethyl)amino)-4- oxobutanoic acid | 556.3 | 557.4 |
| 538 | 526 | 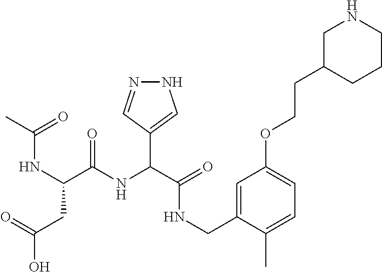 [SMILES: CC(=O)N[C@@H](CC(=O)O)C(=O)NC(C(=O)NCc1cc(OCCC2CCCNC2)ccc1C)c1cn[nH]c1] | (3S)-3-acetamido-4-((2-((2- methyl-5-(2-(piperidin-3- yl)ethoxy)benzyl)amino)-2- oxo-1-(1H-pyrazol-4- yl)ethyl)amino)-4-oxobutanoic acid | 528.3 | 529.4 |
| 539 | 527 | 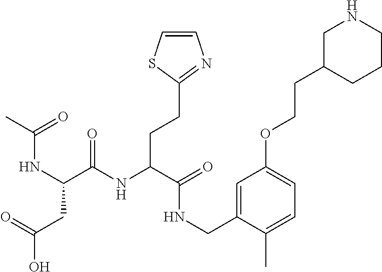 [SMILES: CC(=O)N[C@@H](CC(=O)O)C(=O)NC(CCc1nccs1)C(=O)NCc1cc(OCCC2CCCNC2)ccc1C] | (3S)-3-acetamido-4-((1-((2- methyl-5-(2-(piperidin-3- yl)ethoxy)benzyl)amino)-1- oxo-4-(thiazol-2-yl)butan-2- yl)amino)-4-oxobutanoic acid | 573.3 | 574.6 |
| 540 | 528 | 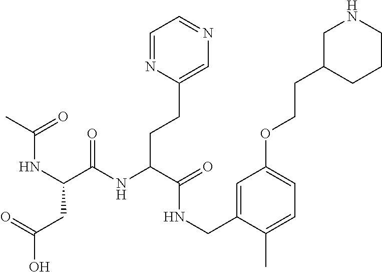 [SMILES: CC(=O)N[C@@H](CC(=O)O)C(=O)NC(CCc1cnccn1)C(=O)NCc1cc(OCCC2CCCNC2)ccc1C] | (3S)-3-acetamido-4-((1-((2- methyl-5-(2-(piperidin-3- yl)ethoxy)benzyl)amino)-1- oxo-4-(pyrazin-2-yl)butan-2- yl)amino)-4-oxobutanoic acid | 568.3 | 569.3 |

### benzyl ((12S)-5⁴-methyl-8,11,14-trioxo-9-(2-(pyridin-4-yl)ethyl)-4-oxa-7,10-diaza-1(3,1)-piperidina-5(1,3)-benzenacyclotetradecaphane-12-yl)carbamate (336)

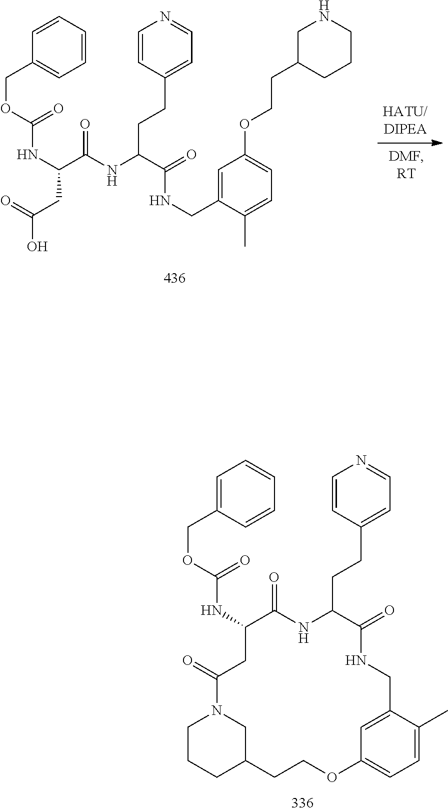

Macrocyclization was achieved according to general procedure A with 606 mg amino acid 436. Reaction mixtures were diluted with some methanol and purified via reversed phase column chromatography (RP18, water/acetonitrile gradient) and HPLC.

Formula: C₃₆₁H₄₃₁N₅O₆, exact mass: 641.3, found: 642.3 [M+H]⁺

The examples in the following table were prepared similar to the procedure described for compound 336:

TABLE A-37

| Cpd | From | | | Exact | [M + H]⁺ |
|---|---|---|---|---|---|
| no | Cpd | structure | name | mass | found |
| 549 | 545 | 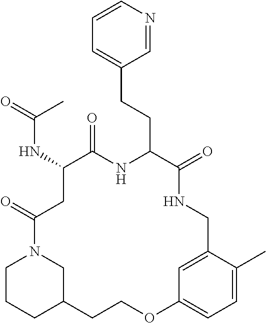 [SMILES: CC(=O)N[C@H]1CC(=O)N2CCCC(CCOc3ccc(C)c(c3)CNC(=O)C(CCc3cccnc3)NC1=O)C2] | N-((12S)-5⁴-methyl-8,11,14- trioxo-9-(2-(pyridin-3-yl)ethyl)- 4-oxa-7,10-diaza-1(3,1)- piperidina-5(1,3)- benzenacyclotetradecaphane- 12-yl)acetamide | 549 | 550 |
| 550 | 546 | 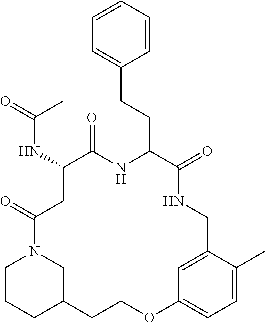 [SMILES: CC(=O)N[C@H]1CC(=O)N2CCCC(CCOc3ccc(C)c(c3)CNC(=O)C(CCc3ccccc3)NC1=O)C2] | N-((12S)-5⁴-methyl-8,11,14- trioxo-9-phenethyl-4-oxa-7,10- diaza-1(3,1)-piperidina-5(1,3)- benzenacyclotetradecaphane- 12-yl)acetamide | 548 | 549 |
| 551 | 547 | 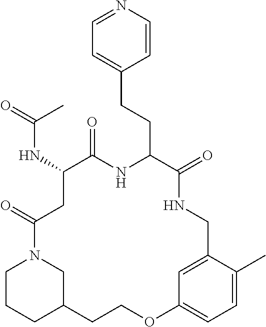 [SMILES: CC(=O)N[C@H]1CC(=O)N2CCCC(CCOc3ccc(C)c(c3)CNC(=O)C(CCc3ccncc3)NC1=O)C2] | N-((12S)-5⁴-methyl-8,11,14- trioxo-9-(2-(pyridin-4-yl)ethyl)- 4-oxa-7,10-diaza-1(3,1)- piperidina-5(1,3)- benzenacyclotetradecaphane- 12-yl)acetamide | 549 | 550 |
| 552 | 548 | 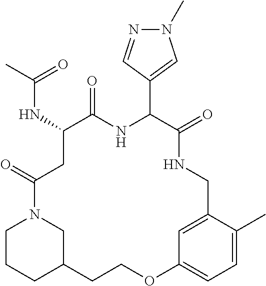 [SMILES: CC(=O)N[C@H]1CC(=O)N2CCCC(CCOc3ccc(C)c(c3)CNC(=O)C(c3cnn(C)c3)NC1=O)C2] | N-((12S)-5⁴-methyl-9-(1- methyl-1H-pyrazol-4-yl)- 8,11,14-trioxo-4-oxa-7,10- diaza-1(3,1)-piperidina-5(1,3)- benzenacyclotetradecaphane- 12-yl)acetamide | 524 | 525 |
| 553 | 529 | 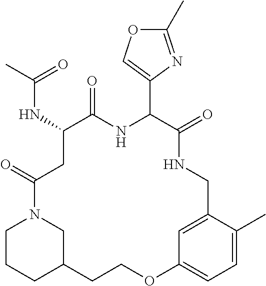 [SMILES: CC(=O)N[C@H]1CC(=O)N2CCCC(CCOc3ccc(C)c(c3)CNC(=O)C(c3coc(C)n3)NC1=O)C2] | N-((12S)-5⁴-methyl-9-(2- methyloxazol-4-yl)-8,11,14- trioxo-4-oxa-7, 10-diaza-1(3,1)- piperidina-5(1,3)- benzenacyclotetradecaphane- 12-yl)acetamide | 525 | 526 |
| 554 | 530 | 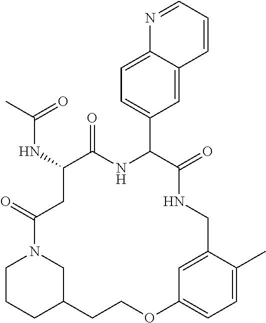 [SMILES: CC(=O)N[C@H]1CC(=O)N2CCCC(CCOc3ccc(C)c(c3)CNC(=O)C(c3ccc4ncccc4c3)NC1=O)C2] | N-((12S)-5⁴-methyl-8,11,14- trioxo-9-(quinolin-6-yl)-4-oxa- 7,10-diaza-1(3,1)-piperidina- 5(1,3)- benzenacyclotetradecaphane- 12-yl)acetamide | 571 | 572 |
| 555 | 531 | 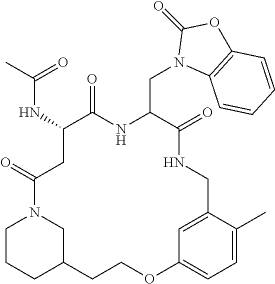 [SMILES: CC(=O)N[C@H]1CC(=O)N2CCCC(CCOc3ccc(C)c(c3)CNC(=O)C(Cn3c(=O)oc4ccccc43)NC1=O)C2] | N-((12S)-5⁴-methyl-8,11,14- trioxo-9-((2- oxobenzo[d]oxazol-3(2H)- yl)methyl)-4-oxa-7,10-diaza- 1(3,1)-piperidina-5(1,3)- benzenacyclotetradecaphane- 12-yl)acetamide | 591 | 592 |
| 556 | 532 | 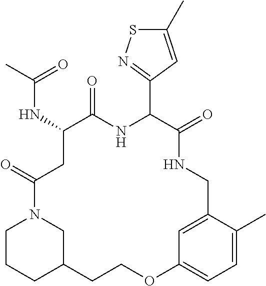 [SMILES: CC(=O)N[C@H]1CC(=O)N2CCCC(CCOc3ccc(C)c(c3)CNC(=O)C(c3cc(C)sn3)NC1=O)C2] | N-((12S)-5⁴-methyl-9-(5- methylisothiazol-3-yl)-8,11,14- trioxo-4-oxa-7,10-diaza-1(3,1)- piperidina-5(1,3)- benzenacyclotetradecaphane- 12-yl)acetamide | 541 | 542 |
| 557 | 533 | 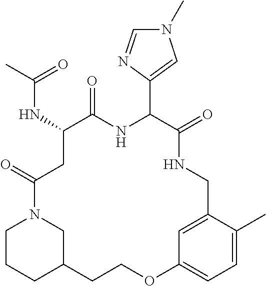 [SMILES: CC(=O)N[C@H]1CC(=O)N2CCCC(CCOc3ccc(C)c(c3)CNC(=O)C(c3cn(C)cn3)NC1=O)C2] | N-((12S)-5⁴-methyl-9-(1- methyl-1H-imidazol-4-yl)- 8,11,14-trioxo-4-oxa-7,10- diaza-1(3,1)-piperidina-5(1,3)- benzenacyclotetradecaphane- 12-yl)acetamide | 524 | 525 |
| 558 | 534 | 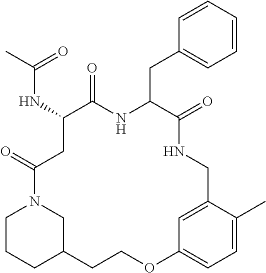 [SMILES: CC(=O)N[C@H]1CC(=O)N2CCCC(CCOc3ccc(C)c(c3)CNC(=O)C(Cc3ccccc3)NC1=O)C2] | N-((12S)-9-benzyl-5⁴-methyl- 8,11,14-trioxo-4-oxa-7,10- diaza-1(3,1)-piperidina-5(1,3)- benzenacyclotetradecaphane- 12-yl)acetamide | 534 | 535 |
| 559 | 535 | 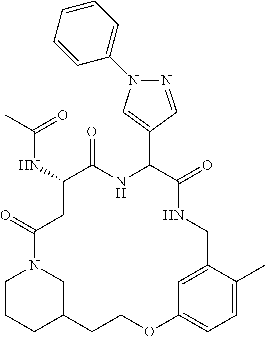 [SMILES: CC(=O)N[C@H]1CC(=O)N2CCCC(CCOc3ccc(C)c(c3)CNC(=O)C(c3cnn(-c4ccccc4)c3)NC1=O)C2] | N-((12S)-5⁴-methyl-8,11,14- trioxo-9-(1-phenyl-1H-pyrazol- 4-yl)-4-oxa-7,10-diaza-1(3,1)- piperidina-5(1,3)- benzenacyclotetradecaphane- 12-yl)acetamide | 586 | 587 |
| 560 | 536 | 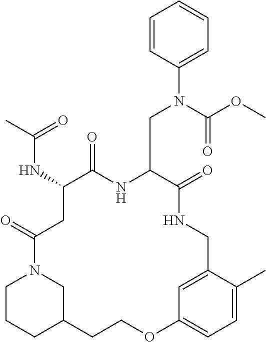 [SMILES: COC(=O)N(CC1NC(=O)[C@@H](NC(C)=O)CC(=O)N2CCCC(CCOc3ccc(C)c(c3)CNC1=O)C2)c1ccccc1] | methyl (((12S)-12-acetamido- 5⁴-methyl-8,11,14-trioxo-4-oxa- 7,10-diaza-1(3,1)-piperidina- 5(1,3)- benzenacyclotetradecaphane- 9-yl)methyl)(phenyl)carbamate | 607 | 608 |
| 561 | 537 | 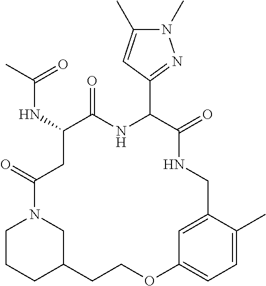 [SMILES: CC(=O)N[C@H]1CC(=O)N2CCCC(CCOc3ccc(C)c(c3)CNC(=O)C(c3cc(C)n(C)n3)NC1=O)C2] | N-((12S)-9-(1,5-dimethyl-1H- pyrazol-3-yl)-5⁴-methyl- 8,11,14-trioxo-4-oxa-7,10- diaza-1(3,1)-piperidina-5(1,3)- benzenacyclotetradecaphane- 12-yl)acetamide | 538 | 539 |
| 562 | 538 | 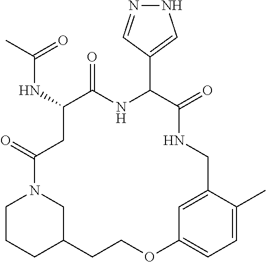 [SMILES: CC(=O)N[C@H]1CC(=O)N2CCCC(CCOc3ccc(C)c(c3)CNC(=O)C(c3cn[nH]c3)NC1=O)C2] | N-((12S)-5⁴-methyl-8,11,14- trioxo-9-(1H-pyrazol-4-yl)-4- oxa-7,10-diaza-1(3,1)- piperidina-5(1,3)- benzenacyclotetradecaphane- 12-yl)acetamide | 510 | 511 |
| 563 | 539 | 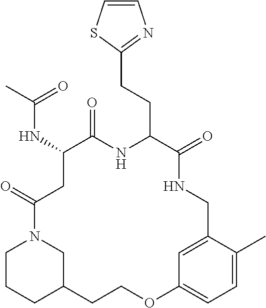 [SMILES: CC(=O)N[C@H]1CC(=O)N2CCCC(CCOc3ccc(C)c(c3)CNC(=O)C(CCc3nccs3)NC1=O)C2] | N-((12S)-5⁴-methyl-8,11,14- trioxo-9-(2-(thiazol-2-yl)ethyl)- 4-oxa-7,10-diaza-1(3,1)- piperidina-5(1,3)- benzenacyclotetradecaphane- 12-yl)acetamide | 555 | 556 |
| 564 | 540 | 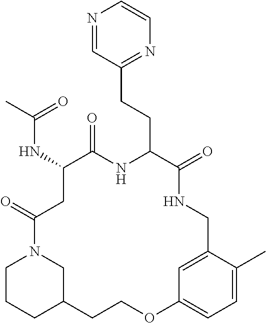 [SMILES: CC(=O)N[C@H]1CC(=O)N2CCCC(CCOc3ccc(C)c(c3)CNC(=O)C(CCc3cnccn3)NC1=O)C2] | N-((12S)-5⁴-methyl-8,11,14- trioxo-9-(2-(pyrazin-2-yl)ethyl)- 4-oxa-7,10-diaza-1(3,1)- piperidina-5(1,3)- benzenacyclotetradecaphane- 12-yl)acetamide | 550 | 551 |

### Example A-32: Preparation of Macrocyclic Compounds 338, 342-344, 437 439, 443 and 444

### ((12S)-12-amino-5⁴-methyl-8,11,14-trioxo-9-(2-(pyridin-4-yl)ethyl)-4-oxa-7,10-diaza-1(3,1)-piperidina-5(1,3)-benzenacyclotetradecaphane-12-yl) (437)

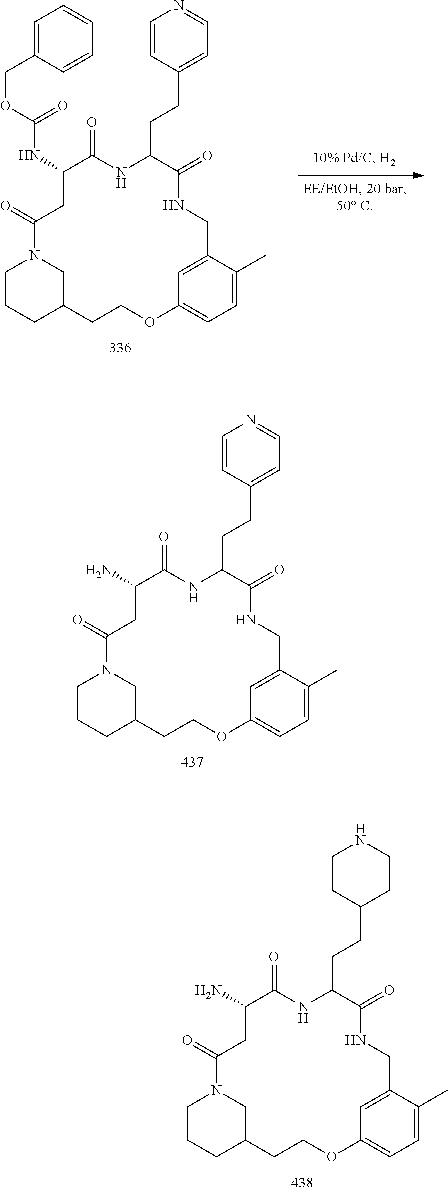 [SMILES: Cc1ccc2cc1CNC(=O)C(CCc1ccncc1)NC(=O)[C@@H](NC(=O)OCc1ccccc1)CC(=O)N1CCCC(CCO2)C1 ; "EE/EtOH, 20 bar,50° C." ; Cc1ccc2cc1CNC(=O)C(CCc1ccncc1)NC(=O)[C@@H](N)CC(=O)N1CCCC(CCO2)C1 ; Cc1ccc2cc1CNC(=O)C(CCC1CCNCC1)NC(=O)[C@@H](N)CC(=O)N1CCCC(CCO2)C1]

Following general procedure C using 190 mg Cbz-protected amine 336 were dissolved ethyl acetate/ethanol (1:1). H-Cube conditions: 1 ml/min, 50° C., 20 bar H₂. After removing of volatiles under reduced pressure the product was used without further purification. Some piperidyl side product 438 was removed after the following step.

437—Formula: C₂₈H₃₇N₅O₄, exact mass: 507.3, found: 508.3 [M+H]⁺

438—Formula: C₂₈H₄₃N₅O₄, exact mass: 513.3, found: 514.3 [M+H]⁺

### N-((12S)-9-(2-(1-acetylpiperidin-4-yl)ethyl)-5⁴-methyl-8,11,14-trioxo-4-oxa-7,10-diaza-1(3,1)-piperidina-5(1,3)-benzenacyclotetradecaphane-12-yl)acetamide (338)

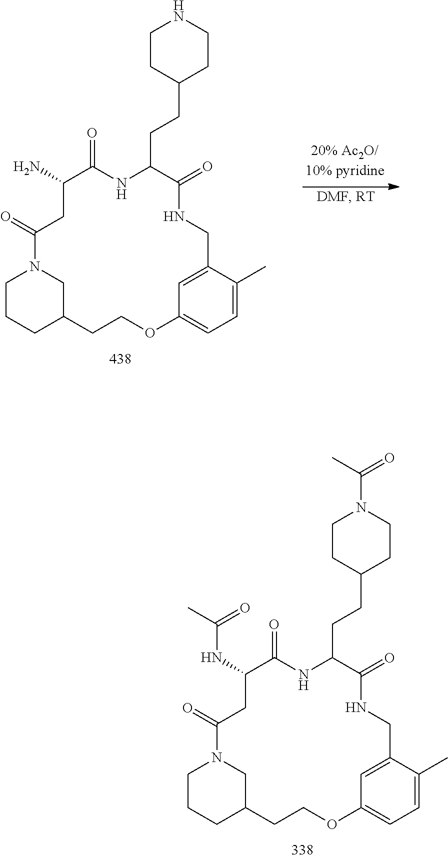

Both free amines in compound 438 were acetylated by dissolving amine 438 in Ac₂O/pyridine/DMF (20/10/70) at room temperature. Purification of the reaction mixture was achieved by HPLC to give compound 338.

Formula: C₃₂H₄₇N₅O₆, exact mass: 597.4, found: 598.4 [M+H]⁺

### Amide Derivatizations of Compound 437 According to General Procedure B

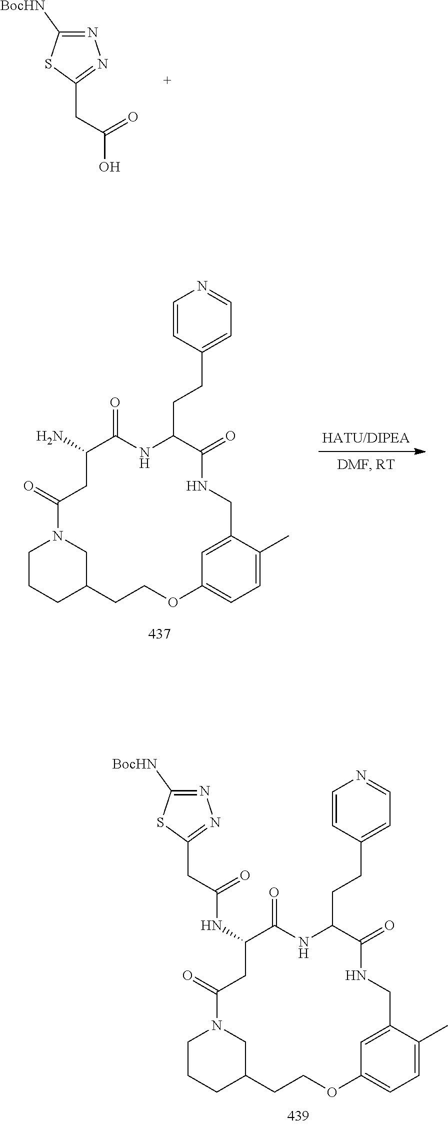 [SMILES: CC(C)(C)OC(=O)Nc1nnc(CC(=O)O)s1 ; Cc1ccc2cc1CNC(=O)C(CCc1ccncc1)NC(=O)[C@@H](N)CC(=O)N1CCCC(CCO2)C1 ; Cc1ccc2cc1CNC(=O)C(CCc1ccncc1)NC(=O)[C@@H](NC(=O)Cc1nnc(NC(=O)OC(C)(C)C)s1)CC(=O)N1CCCC(CCO2)C1]

Amine 437 (32 mg) was coupled with carboxylic acids according to general procedure B to give amides disclosed in table below as exemplified for derivative 439. Purifications were achieved by HPLC or reversed phase flash chromatography.

TABLE A-38

| Cpd | | | Exact | [M + H]⁺ |
|---|---|---|---|---|
| no | structure | name | mass | found |
| 439 | 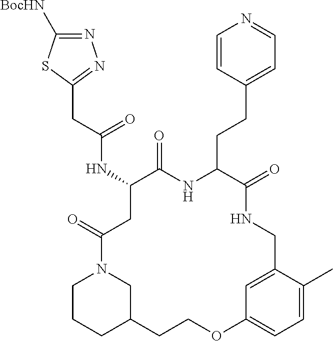 [SMILES: Cc1ccc2cc1CNC(=O)C(CCc1ccncc1)NC(=O)[C@@H](NC(=O)Cc1nnc(NC(=O)OC(C)(C)C)s1)CC(=O)N1CCCC(CCO2)C1] | tert-butyl (5-(2-(((12S)-5⁴-methyl- 8,11,14-trioxo-9-(2-(pyridin-4- yl)ethyl)-4-oxa-7,10-diaza-1(3,1)- piperidina-5(1,3)- benzenacyclotetradecaphane-12- yl)amino)-2-oxoethyl)-1,3,4- thiadiazol-2-yl)carbamate | 748.3 C₃₇H₄₈ N₈O₇S | 749.3 |
| 443 | 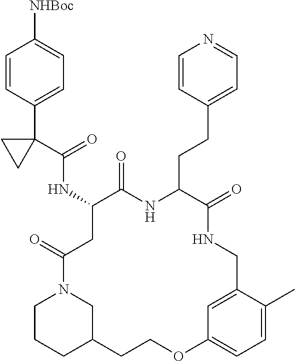 [SMILES: Cc1ccc2cc1CNC(=O)C(CCc1ccncc1)NC(=O)[C@@H](NC(=O)C1(c3ccc(NC(=O)OC(C)(C)C)cc3)CC1)CC(=O)N1CCCC(CCO2)C1] | tert-butyl (4-(1-(((12S)-5⁴-methyl- 8,11,14-trioxo-9-(2-(pyridin-4- yl)ethyl)-4-oxa-7,10-diaza-1(3,1)- piperidina-5(1,3)- benzenacyclotetradecaphane-12- yl)carbamoyl)cyclopropyl)phenyl) carbamate | 766.4 C₄₃H₅₄ N₆O₇ | 767.4 |
| 444 | 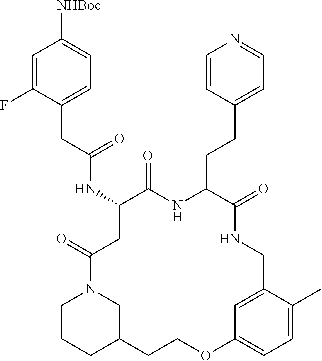 [SMILES: Cc1ccc2cc1CNC(=O)C(CCc1ccncc1)NC(=O)[C@@H](NC(=O)Cc1ccc(NC(=O)OC(C)(C)C)cc1F)CC(=O)N1CCCC(CCO2)C1] | tert-butyl (3-fluoro-4-(2-(((12S)-5⁴- methyl-8,11,14-trioxo-9-(2- (pyridin-4-yl)ethyl)-4-oxa-7,10- diaza-1(3,1)-piperidina-5(1,3)- benzenacyclotetradecaphane-12- yl)amino)-2- oxoethyl)phenyl)carbamate | 758.4 C₄₁H₅₁F N₆O₇ | 759.4 |

### Removal of Boc-Protecting Groups

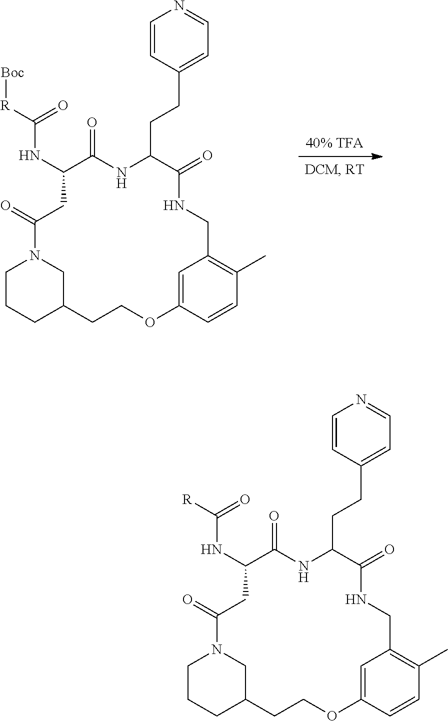

Boc protecting groups were cleaved from the compound according to general procedure D stirring compounds in 5 ml 40% TFA in DCM at room temperature. After deprotection was complete solvent was removed and the crude product was purified by reversed phase HPLC.

TABLE A-39

| Cpd | From | | | Exact | [M + H]⁺ |
|---|---|---|---|---|---|
| no | cpd | structure | name | mass | found |
| 342 | 439 | 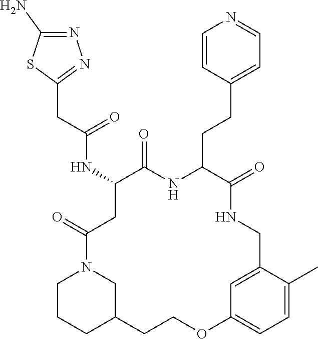 [SMILES: Cc1ccc2cc1CNC(=O)C(CCc1ccncc1)NC(=O)[C@@H](NC(=O)Cc1nnc(N)s1)CC(=O)N1CCCC(CCO2)C1] | 2-(5-amino-1,3,4-thiadiazol-2- yl)-N-((12S)-5⁴-methyl-8,11,14- trioxo-9-(2-(pyridin-4-yl)ethyl)-4- oxa-7,10-diaza-1(3,1)- piperidina-5(1,3)- benzenacyclotetradecaphane- 12-yl)acetamide | 648.3 C₃₂H₄₀ N₈O₅S | 649.3 |
| 343 | 443 | 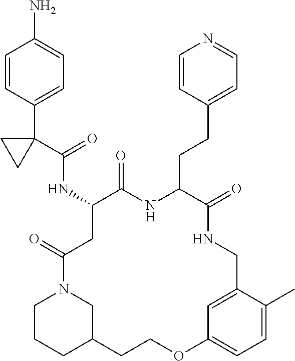 [SMILES: Cc1ccc2cc1CNC(=O)C(CCc1ccncc1)NC(=O)[C@@H](NC(=O)C1(c3ccc(N)cc3)CC1)CC(=O)N1CCCC(CCO2)C1] | 1-(4-aminophenyl)-N-((12S)-5⁴- methyl-8,11,14-trioxo-9-(2- (pyridin-4-yl)ethyl)-4-oxa-7,10- diaza-1(3,1)-piperidina-5(1,3)- benzenacyclotetradecaphane- 12-yl)cyclopropane-1- carboxamide | 666.4 C₃₈H₄₆ N₆O₅ | 667.4 |
| 344 | 444 | 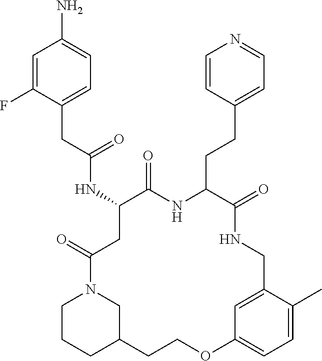 [SMILES: Cc1ccc2cc1CNC(=O)C(CCc1ccncc1)NC(=O)[C@@H](NC(=O)Cc1ccc(N)cc1F)CC(=O)N1CCCC(CCO2)C1] | 2-(4-amino-2-fluorophenyl)-N- ((12S)-5⁴-methyl-8,11,14-trioxo- 9-(2-(pyridin-4-yl)ethyl)-4-oxa- 7,10-diaza-1(3,1)-piperidina- 5(1,3)- benzenacyclotetradecaphane- 12-yl)acetamide | 658.3 C₃₆H₄₃ FN₆O₅ | 659.4 |

### Example A-33: Preparation of Macrocyclic Compound 348

### 2,2-dimethyl-N-((9R,12R)-5⁴-methyl-8,11,14-trioxo-9-phenethyl-4-oxa-7,10-diaza-1(3,1)-piperidina-5(1,3)-benzenacyclotetradecaphane-12-yl)-3-(tritylthio)propanamide (450)

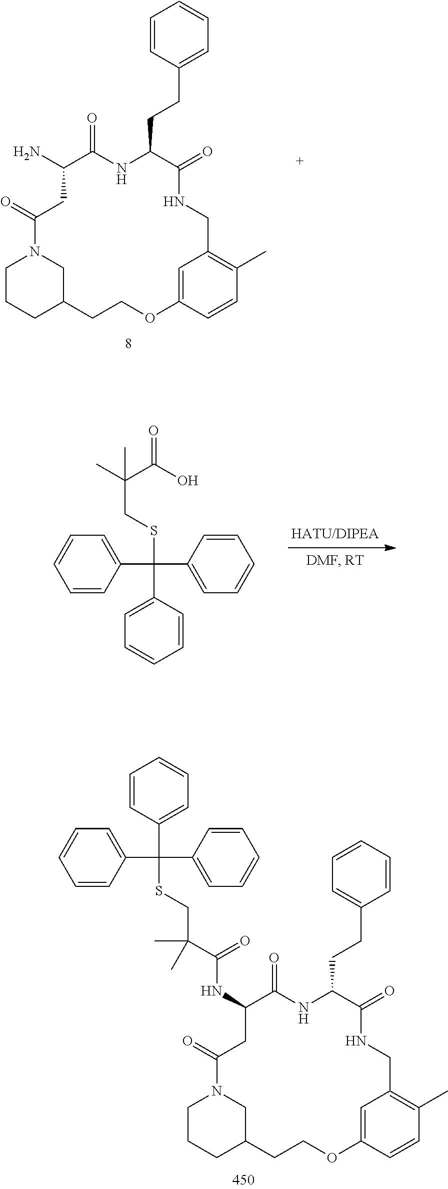 [SMILES: Cc1ccc2cc1CNC(=O)[C@H](CCc1ccccc1)NC(=O)[C@@H](N)CC(=O)N1CCCC(CCO2)C1 ; CC(C)(CSC(c1ccccc1)(c1ccccc1)c1ccccc1)C(=O)O ; Cc1ccc2cc1CNC(=O)[C@@H](CCc1ccccc1)NC(=O)[C@H](NC(=O)C(C)(C)CSC(c1ccccc1)(c1ccccc1)c1ccccc1)CC(=O)N1CCCC(CCO2)C1]

Amine 8 (100 mg) was coupled with the carboxylic acid according to general procedure B to give protected thiol 450. Purification was achieved by reversed phase HPLC.

Formula: C₃₂H₄₇N₅O₆, exact mass: 597.4, found: 598.4 [M+H]⁺

### 3-mercapto-2,2-dimethyl-N-((9S,12S)-5⁴-methyl-8,11,14-trioxo-9-phenethyl-4-oxa-7,10-diaza-1(3,1)-piperidina-5(1,3)-benzenacyclotetradecaphane-12-yl)propanamide (348)

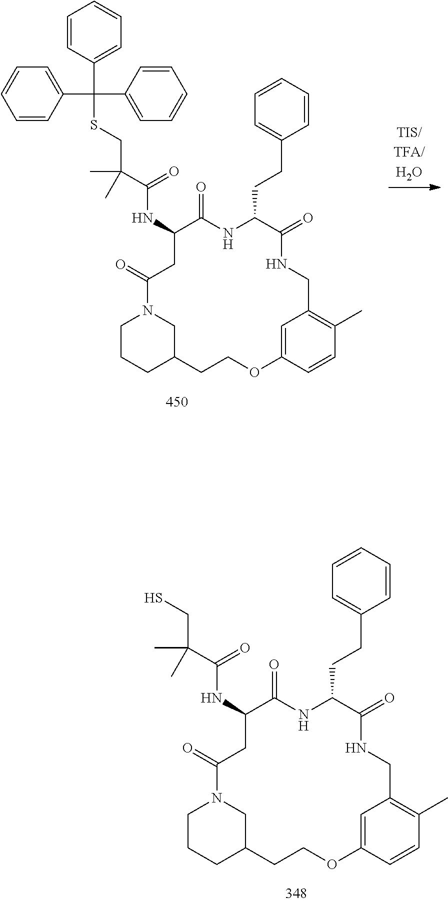

Compound 450 (140 mg) was dissolved in 1 ml TIS/TFA/H₂O (10/2/2) at room temperature and stirred for 1h. Purification by reversed phase column chromatography (RP18, water/acetonitrile gradient).

Formula: C₃₄H₄₆N₄O₆, exact mass: 622.3, found: 623.5 [M+H]⁺

### Example A-34: Preparation of Macrocyclic Compound 355

### ethyl 4-(3-(2-(3-((2-(((benzyloxy)carbonyl)amino)-2-(1-methyl-1H-pyrazol-4-yl)acetamido)methyl)-4,5-dimethylphenoxy)ethyl)piperidin-1-yl)-4-oxobutanoate (470)

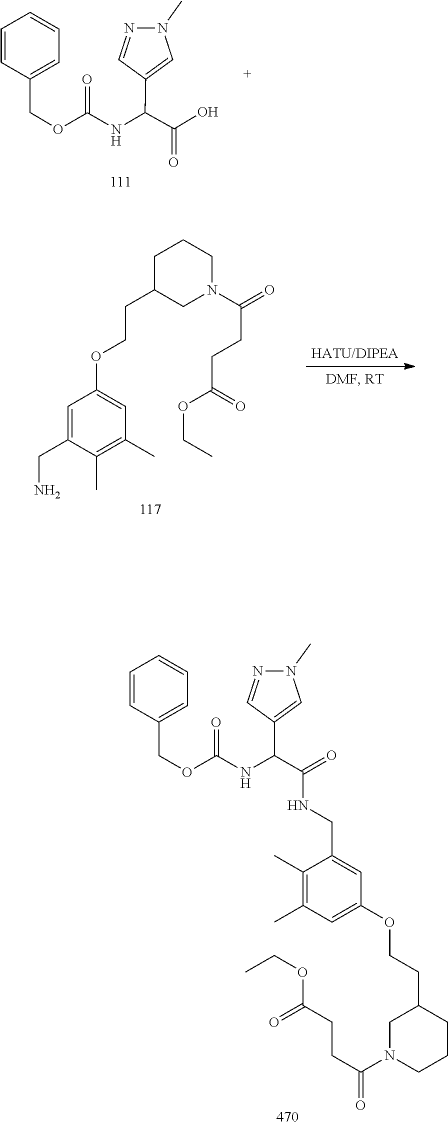 [SMILES: Cn1cc(C(NC(=O)OCc2ccccc2)C(=O)O)cn1 ; CCOC(=O)CCC(=O)N1CCCC(CCOc2cc(C)c(C)c(CN)c2)C1 ; CCOC(=O)CCC(=O)N1CCCC(CCOc2cc(C)c(C)c(CNC(=O)C(NC(=O)OCc3ccccc3)c3cnn(C)c3)c2)C1]

162 mg amine 117 and 100 mg carboxylic acid 111 were coupled according to general procedure B to give compound 470. Purification was achieved by reversed phase column chromatography (RP18, water/acetonitrile gradient).

Formula: C₃₆H₄₇N₅O₇, exact mass: 661.3, found: 662.5 [M+H]⁺

### ethyl 4-(3-(2-(3-((2-amino-2-(1-methyl-1H-pyrazol-4-yl)acetamido)methyl)-4,5-dimethylphenoxy)ethyl)piperidin-1-yl)-4-oxobutanoate (471)

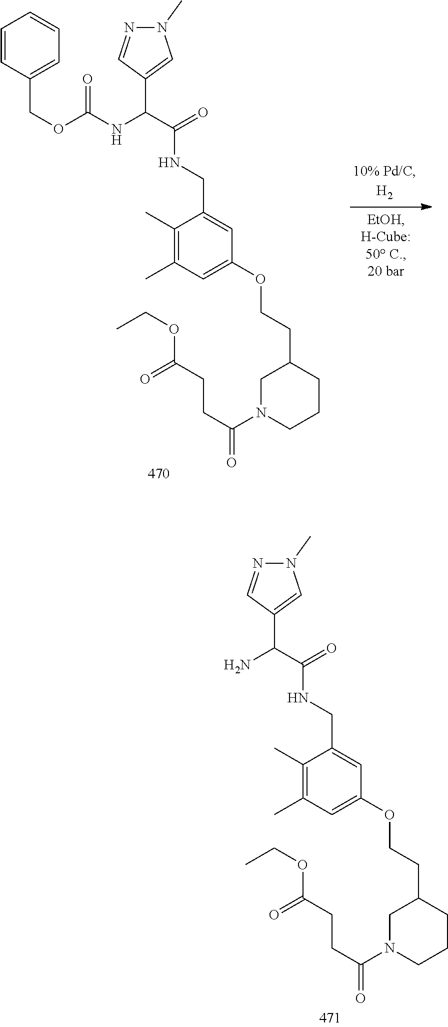 [SMILES: CCOC(=O)CCC(=O)N1CCCC(CCOc2cc(C)c(C)c(CNC(=O)C(NC(=O)OCc3ccccc3)c3cnn(C)c3)c2)C1 ; CCOC(=O)CCC(=O)N1CCCC(CCOc2cc(C)c(C)c(CNC(=O)C(N)c3cnn(C)c3)c2)C1]

Cbz-protection group was removed according to general procedure C in EtOH at 50° C. and 20 bar H₂ pressure. Solvent was removed under reduced pressure to give a product pure enough to be used in the next reaction.

Formula: C₂₈H₄₁N₅O₅, exact mass: 527.3, found: 528.4 [M+H]⁺

### 4-(3-(2-(3-((2-amino-2-(1-methyl-1H-pyrazol-4-yl)acetamido)methyl)-4,5-dimethylphenoxy)ethyl) piperidin-1-yl)-4-oxobutanoic acid (472)

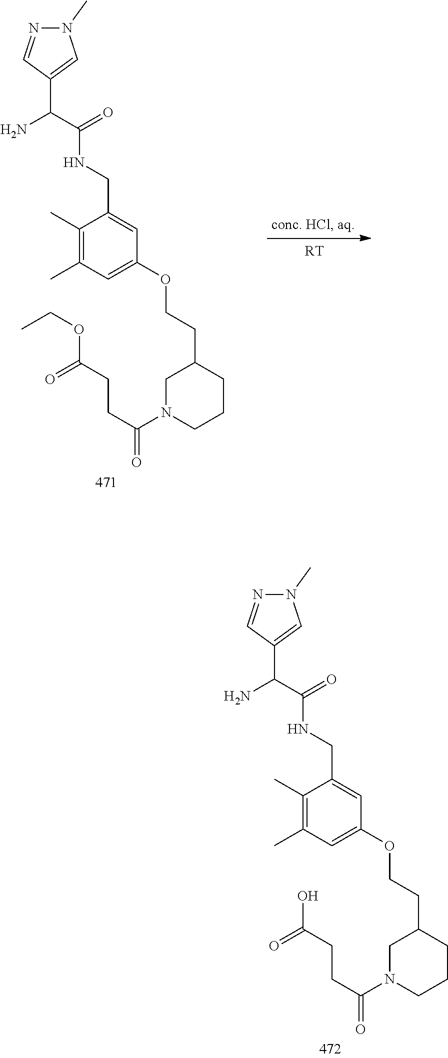 [SMILES: CCOC(=O)CCC(=O)N1CCCC(CCOc2cc(C)c(C)c(CNC(=O)C(N)c3cnn(C)c3)c2)C1 ; "RT" ; Cc1cc(OCCC2CCCN(C(=O)CCC(=O)O)C2)cc(CNC(=O)C(N)c2cnn(C)c2)c1C]

The crude ester 471 was dissolved in concentrated aqueous HCl solution at room temperature. The solution was stirred until the reaction was complete. Volatiles were removed and dissolved in acetonitrile. After drying the crude was used in the following cyclization.

Formula: C₂₆H₃₇N₅O₅, exact mass: 499.3, found: 500.2 [M+H]⁺

### 5⁴,5⁵-dimethyl-9-(1-methyl-1H-pyrazol-4-yl)-4-oxa-7,10-diaza-1(3,1)-piperidina-5(1,3)-benzenacyclotetradecaphane-8,11,14-trione (355)

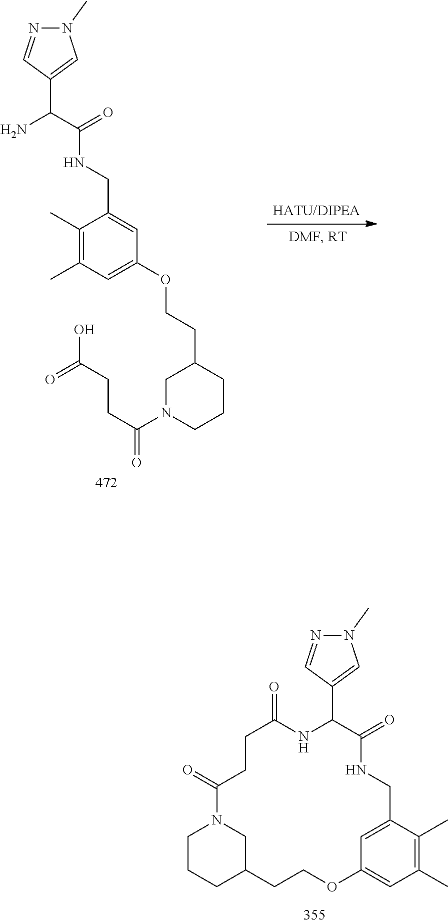 [SMILES: Cc1cc(OCCC2CCCN(C(=O)CCC(=O)O)C2)cc(CNC(=O)C(N)c2cnn(C)c2)c1C ; Cc1cc2cc(c1C)CNC(=O)C(c1cnn(C)c1)NC(=O)CCC(=O)N1CCCC(CCO2)C1]

Macrocyclization was achieved according to general procedure A with 75 mg amino acid 472. Reaction mixtures were diluted with some methanol and purified via reversed phase column chromatography (RP18, water/acetonitrile gradient) and HPLC.

Formula: C₂₆H₃₅N₅O₄, exact mass: 481.3, found: 482.3 [M+H]⁺

### Example A-35: Preparation of Macrocyclic Compound 356

### tert-butyl 3-(2-(3-((2-(((benzyloxy)carbonyl)amino)-2-(1-methyl-1H-pyrazol-4-yl)acetamido)methyl)-4-methylphenoxy)ethyl)piperidine-1-carboxylate (473)

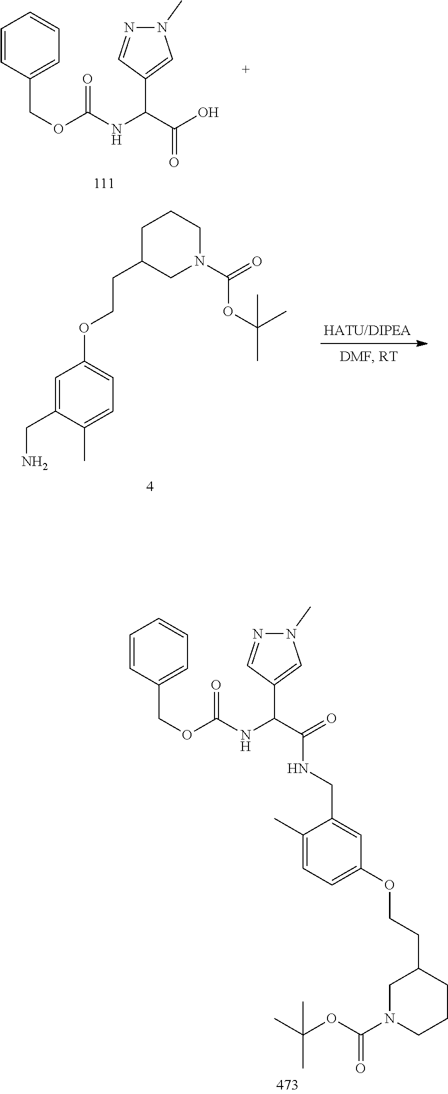 [SMILES: Cn1cc(C(NC(=O)OCc2ccccc2)C(=O)O)cn1 ; Cc1ccc(OCCC2CCCN(C(=O)OC(C)(C)C)C2)cc1CN ; Cc1ccc(OCCC2CCCN(C(=O)OC(C)(C)C)C2)cc1CNC(=O)C(NC(=O)OCc1ccccc1)c1cnn(C)c1]

452 mg amine 4 and 341 mg carboxylic acid 111 were coupled according to general procedure B to give compound 473. Purification was achieved by normal phase column chromatography (silica, cyclohexane/ethyl acetate gradient).

Formula: C₃₄H₄₅N₅O₆, exact mass: 619.3, found: 620.3 [M+H]⁺

### tert-butyl 3-(2-(3-((2-amino-2-(1-methyl-1H-pyrazol-4-yl)acetamido)methyl)-4-methylphenoxy)ethyl)piperidine-1-carboxylate (474)

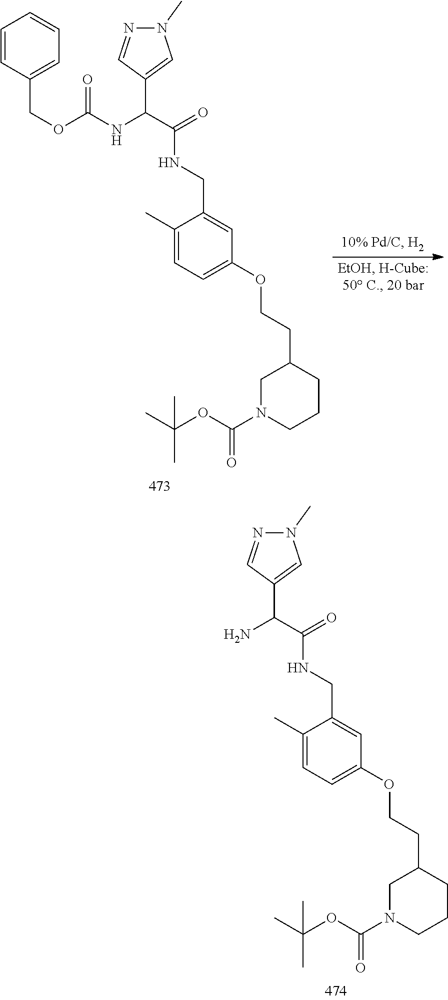 [SMILES: Cc1ccc(OCCC2CCCN(C(=O)OC(C)(C)C)C2)cc1CNC(=O)C(NC(=O)OCc1ccccc1)c1cnn(C)c1 ; Cc1ccc(OCCC2CCCN(C(=O)OC(C)(C)C)C2)cc1CNC(=O)C(N)c1cnn(C)c1]

Cbz-protection group was removed according to general procedure C in EtOH at 50° C. and 20 bar H₂ pressure. Solvent was removed under reduced pressure to give a product pure enough to be used in the next reaction.

Formula: C₂₆H₃₉N₅O₄, exact mass: 485.3, found: 486.3 [M+H]⁺

### tert-butyl 3-(2-(3-((2-(6-ethoxy-6-oxohexanamido)-2-(1-methyl-1H-pyrazol-4-yl)acetamido)methyl)-4-methylphenoxy)ethyl)piperidine-1-carboxylate (475)

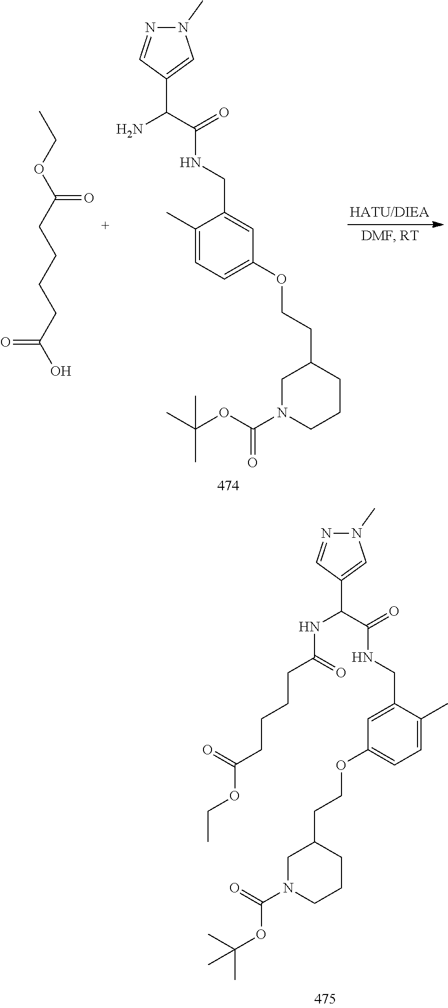 [SMILES: CCOC(=O)CCCCC(=O)O ; Cc1ccc(OCCC2CCCN(C(=O)OC(C)(C)C)C2)cc1CNC(=O)C(N)c1cnn(C)c1 ; CCOC(=O)CCCCC(=O)NC(C(=O)NCc1cc(OCCC2CCCN(C(=O)OC(C)(C)C)C2)ccc1C)c1cnn(C)c1]

100 mg amine 474 and 43 mg 6-ethoxy-6-oxohexanoic acid were coupled according to general procedure B to give compound 475. Purification was achieved by normal phase column chromatography (silica, cyclohexane/ethyl acetate gradient).

Formula: C₃₄H₅₁N₅O₇, exact mass: 641.4, found: 642.4 [M+H]⁺

### 6-((1-(1-methyl-1H-pyrazol-4-yl)-2-((2-methyl-5-(2-(piperidin-3-yl)ethoxy)benzyl)amino)-2-oxoethyl)amino)-6-oxohexanoic acid (476)

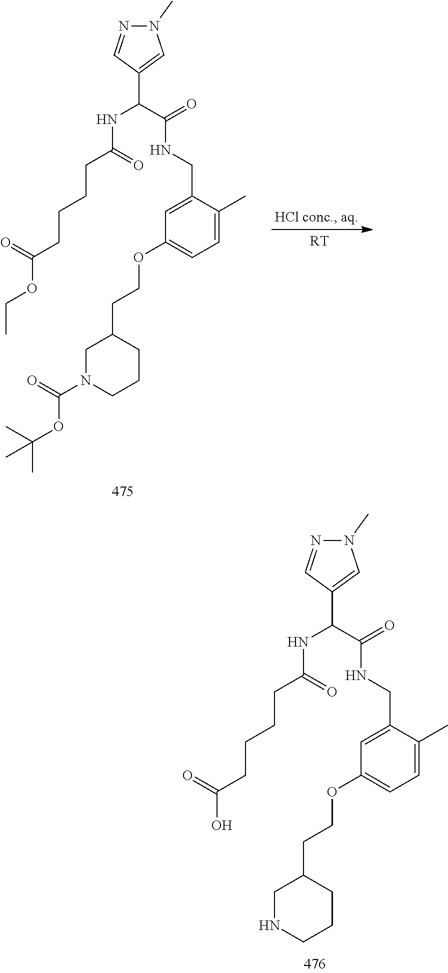 [SMILES: CCOC(=O)CCCCC(=O)NC(C(=O)NCc1cc(OCCC2CCCN(C(=O)OC(C)(C)C)C2)ccc1C)c1cnn(C)c1 ; "RT" ; Cc1ccc(OCCC2CCCNC2)cc1CNC(=O)C(NC(=O)CCCCC(=O)O)c1cnn(C)c1]

Ester 475 was dissolved in concentrated aqueous HCl solution at room temperature. The solution was stirred until the reaction was complete. Volatiles were removed and dissolved in acetonitrile. After drying the crude was used in the following cyclization.

Formula: C₂₇H₃₉N₅O₅, exact mass: 513.3, found: 514.3 [M+H]⁺

### 5⁴-methyl-9-(1-methyl-1H-pyrazol-4-yl)-4-oxa-7,10-diaza-1(3,1)-piperidina-5(1,3)-benzenacyclohexadecaphane-8,11,16-trione (356)

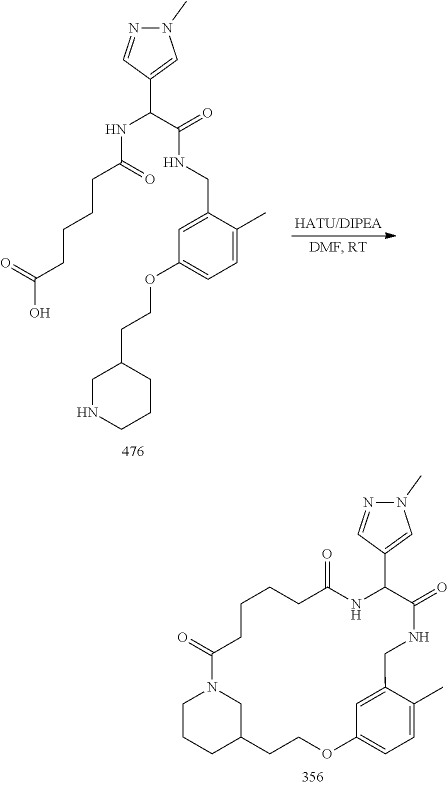 [SMILES: Cc1ccc(OCCC2CCCNC2)cc1CNC(=O)C(NC(=O)CCCCC(=O)O)c1cnn(C)c1 ; Cc1ccc2cc1CNC(=O)C(c1cnn(C)c1)NC(=O)CCCCC(=O)N1CCCC(CCO2)C1]

Macrocyclization was achieved according to general procedure A with 0.11 amino acid 476. Reaction mixtures were diluted with some methanol and purified via HPLC.

Formula: C₂₇H₃₇N₅O₄, exact mass: 495.3, found: 496.3 [M+H]⁺

### Example A-36: Preparation of Macrocyclic Compounds 358-363, 479, 480, and 552 tert-butyl 3-(2-(3-((5S)-5-(2-ethoxy-2-oxoethyl)-8-(1-methyl-1H-pyrazol-4-yl)-3,6,9-trioxo-1-phenyl-2-oxa-4,7,10-triazaundecan-11-yl)-4-methylphenoxy)ethyl)piperidine-1-carboxylate (477)

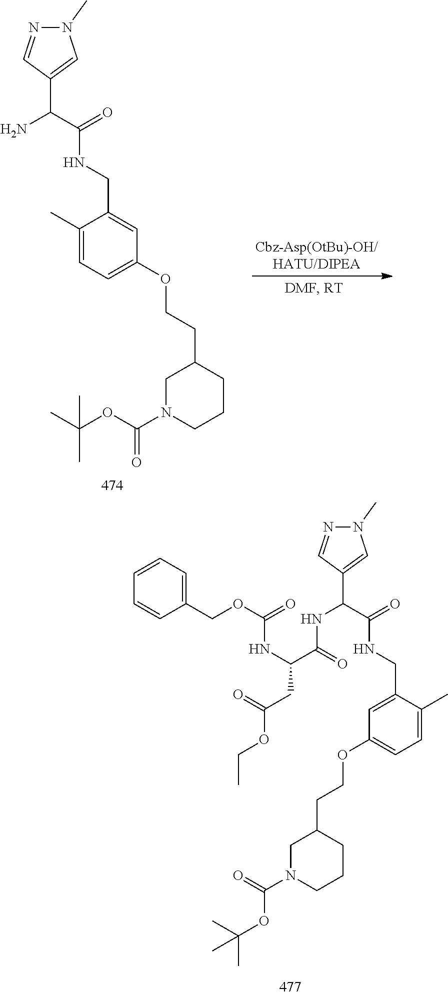 [SMILES: Cc1ccc(OCCC2CCCN(C(=O)OC(C)(C)C)C2)cc1CNC(=O)C(N)c1cnn(C)c1 ; CCOC(=O)C[C@H](NC(=O)OCc1ccccc1)C(=O)NC(C(=O)NCc1cc(OCCC2CCCN(C(=O)OC(C)(C)C)C2)ccc1C)c1cnn(C)c1]

3 g amine 474 and 3 g Cbz-Asp(OtBu)-OH were coupled according to general procedure B to give compound 477. Purification was achieved by normal phase column chromatography (silica, cyclohexane/ethyl acetate gradient).

Formula: C₄₀H₅₄NO₉, exact mass: 762.4, found: 763.5 [M+H]⁺

### (3S)-3-(((benzyloxy)carbonyl)amino)-4-((1-(1-methyl-1H-pyrazol-4-yl)-2-((2-methyl-5-(2-(piperidin-3-yl)ethoxy)benzyl)amino)-2-oxoethyl)amino)-4-oxobutanoic acid (478)

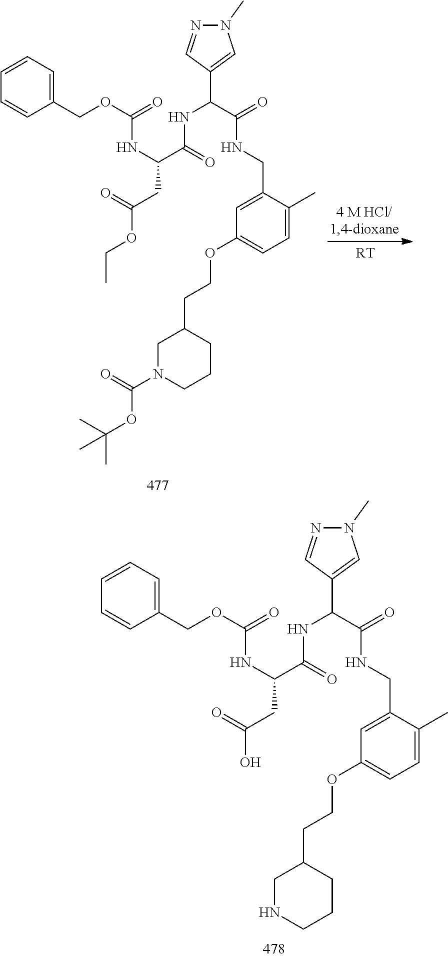 [SMILES: CCOC(=O)C[C@H](NC(=O)OCc1ccccc1)C(=O)NC(C(=O)NCc1cc(OCCC2CCCN(C(=O)OC(C)(C)C)C2)ccc1C)c1cnn(C)c1 ; "RT" ; Cc1ccc(OCCC2CCCNC2)cc1CNC(=O)C(NC(=O)[C@H](CC(=O)O)NC(=O)OCc1ccccc1)c1cnn(C)c1]

4.2 g ester 477 was dissolved in 40 ml 4 M HCl solution in 1,4-dioxane at room temperature. The solution was stirred until the reaction was complete. Volatiles were removed and dissolved in acetonitrile. After drying the crude was used in the following cyclization.

Formula: C₃₃H₄₂N₆O₇, exact mass: 634.3, found: 635.3 [M+H]⁺

### benzyl ((12S)-5⁴-methyl-9-(1-methyl-1H-pyrazol-4-yl)-8,11,14-trioxo-4-oxa-7,10-diaza-1(3,1)-piperidina-5(1,3)-benzenacyclotetradecaphane-12-yl)carbamate (479)

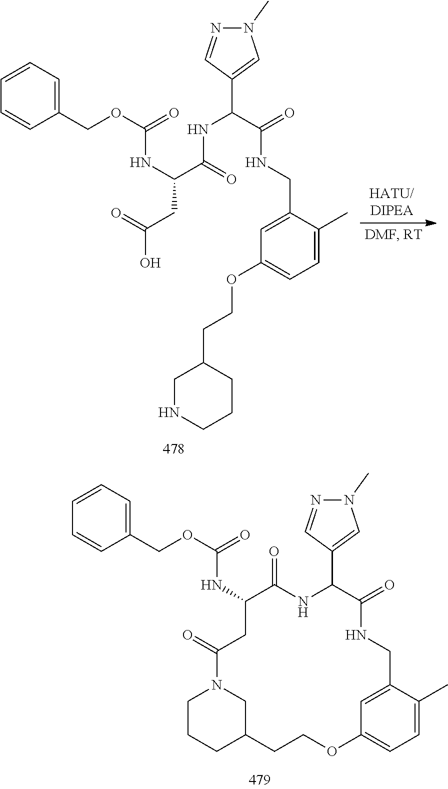 [SMILES: Cc1ccc(OCCC2CCCNC2)cc1CNC(=O)C(NC(=O)[C@H](CC(=O)O)NC(=O)OCc1ccccc1)c1cnn(C)c1 ; Cc1ccc2cc1CNC(=O)C(c1cnn(C)c1)NC(=O)[C@@H](NC(=O)OCc1ccccc1)CC(=O)N1CCCC(CCO2)C1]

Macrocyclization was achieved according to general procedure A with 2.42 g amino acid 478. Saturated NaHCO₃ solution was added to the reaction mixture and this was extracted with ethyl acetate. Organic phases were dried over MgSO₄ and concentrated under reduced pressure. Purification was achieved via normal phase column chromatography (silica, cyclohexane/ethyl acetate gradient).

Formula: C₃₃H₄ON₆O₆, exact mass: 616.3, found: 617.3 [M+H]⁺

### (12S)-12-amino-5⁴-methyl-9-(1-methyl-1H-pyrazol-4-yl)-4-oxa-7,10-diaza-1(3,1)-piperidina-5(1,3)-benzenacyclotetradecaphane-8,11,14-trione (480)

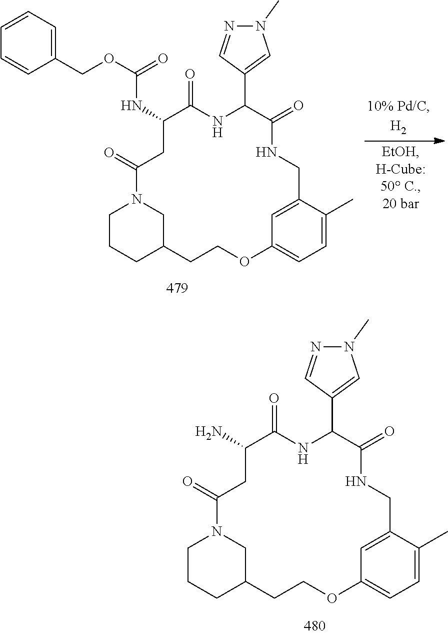 [SMILES: Cc1ccc2cc1CNC(=O)C(c1cnn(C)c1)NC(=O)[C@@H](NC(=O)OCc1ccccc1)CC(=O)N1CCCC(CCO2)C1 ; Cc1ccc2cc1CNC(=O)C(c1cnn(C)c1)NC(=O)[C@@H](N)CC(=O)N1CCCC(CCO2)C1]

Cbz-group was removed according to general procedure C in EtOH at 50° C. and 20 bar H₂ pressure. Solvent was removed under reduced pressure to give a product pure enough to be used in the next reaction.

Formula: C₂₅H₃₄N₆O₄, exact mass: 482.3

### ethyl ((12S)-5⁴-methyl-9-(1-methyl-1H-pyrazol-4-yl)-8,11,14-trioxo-4-oxa-7,10-diaza-1(3,1)-piperidina-5(1,3)-benzenacyclotetradecaphane-12-yl)carbamate (363)

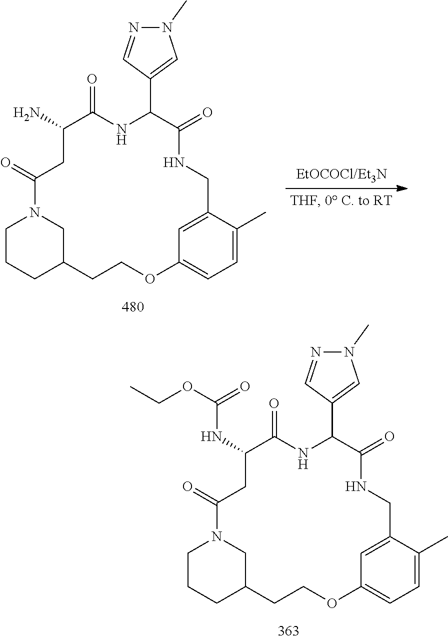 [SMILES: Cc1ccc2cc1CNC(=O)C(c1cnn(C)c1)NC(=O)[C@@H](N)CC(=O)N1CCCC(CCO2)C1 ; CCOC(=O)N[C@H]1CC(=O)N2CCCC(CCOc3ccc(C)c(c3)CNC(=O)C(c3cnn(C)c3)NC1=O)C2]

Compound 363 was synthesized according to general procedure G from 75 mg amine 480. Final purification was achieved by HPLC.

Formula: C₂₈H₃₈N₆O₆, exact mass: 554.3, found: 555.3 [M+H]⁺

### N-((12S)-5⁴-methyl-9-(1-methyl-1H-pyrazol-4-yl)-8,11,14-trioxo-4-oxa-7,10-diaza-1(3,1)-piperidina-5(1,3)-benzenacyclotetradecaphane-12-yl)acetamide (552)

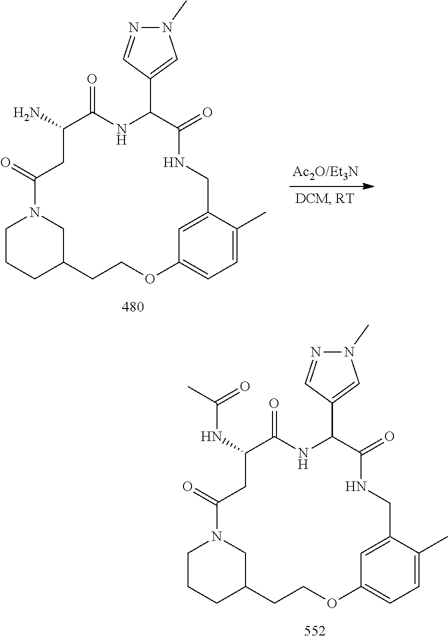 [SMILES: Cc1ccc2cc1CNC(=O)C(c1cnn(C)c1)NC(=O)[C@@H](N)CC(=O)N1CCCC(CCO2)C1 ; CC(=O)N[C@H]1CC(=O)N2CCCC(CCOc3ccc(C)c(c3)CNC(=O)C(c3cnn(C)c3)NC1=O)C2]

Compound 552 was synthesized from 75 mg amine 480. Final purification was achieved by HPLC.

Formula: C₂₇H₃₆N₆O₅, exact mass: 524.3, found: 525.4 [M+H]⁺

### Amide Derivatizations of Compound 480 According to General Procedure B

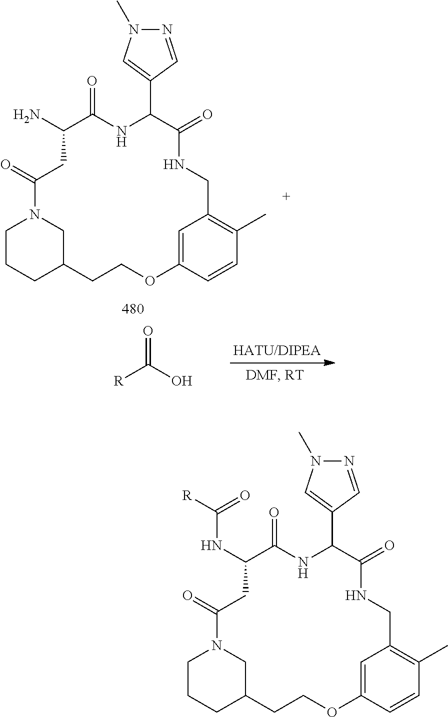

Amine 480 (e.g. 75 mg) was coupled with carboxylic acids according to general procedure B to give amides disclosed in table below. Purifications were achieved by HPLC.

TABLE A-40

| Cpd | | | Exact | [M + H]⁺ |
|---|---|---|---|---|
| no | structure | name | mass | found |
| 358 | 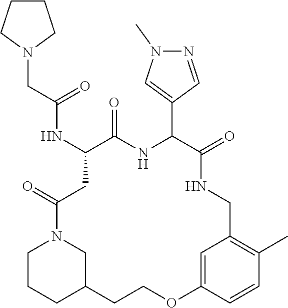 [SMILES: Cc1ccc2cc1CNC(=O)C(c1cnn(C)c1)NC(=O)[C@@H](NC(=O)CN1CCCC1)CC(=O)N1CCCC(CCO2)C1] | N-((12S)-5⁴-methyl-9-(1-methyl-1H- pyrazol-4-yl)-8,11,14-trioxo-4-oxa-7,10- diaza-1(3,1)-piperidina-5(1,3)- benzenacyclotetradecaphane-12-yl)-2- (pyrrolidin-1-yl)acetamide | 593.3 | 594.4 |
| 359 | 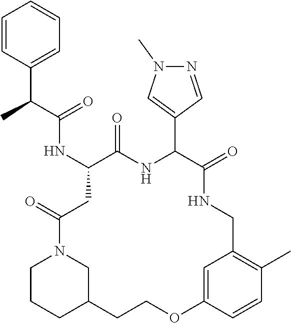 [SMILES: Cc1ccc2cc1CNC(=O)C(c1cnn(C)c1)NC(=O)[C@@H](NC(=O)[C@@H](C)c1ccccc1)CC(=O)N1CCCC(CCO2)C1] | (2S)-N-((12S)-5⁴-methyl-9-(1-methyl- 1H-pyrazol-4-yl)-8,11,14-trioxo-4-oxa- 7,10-diaza-1(3,1)-piperidina-5(1,3)- benzenacyclotetradecaphane-12-yl)-2- phenylpropanamide | 614.3 | 615.4 |
| 360 | 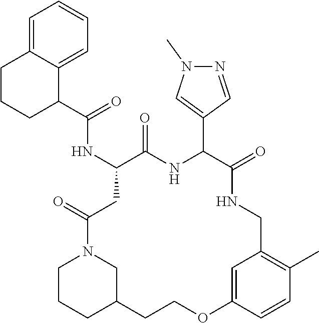 [SMILES: Cc1ccc2cc1CNC(=O)C(c1cnn(C)c1)NC(=O)[C@@H](NC(=O)C1CCCc3ccccc31)CC(=O)N1CCCC(CCO2)C1] | N-((12S)-5⁴-methyl-9-(1-methyl-1H- pyrazol-4-yl)-8,11,14-trioxo-4-oxa-7,10- diaza-1(3,1)-piperidina-5(1,3)- benzenacyclotetradecaphane-12-yl)- 1,2,3,4-tetrahydronaphthalene-1- carboxamide | 640.3 | 641.4 |
| 361 | 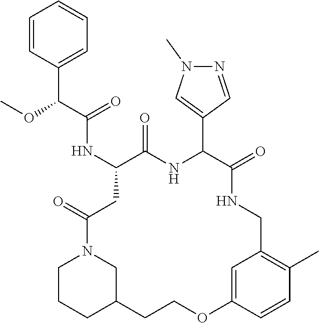 | (2R)-2-methoxy-N-((12S)-5⁴-methyl-9- (1-methyl-1H-pyrazol-4-yl)-8,11,14- trioxo-4-oxa-7,10-diaza-1(3,1)- piperidina-5(1,3)- benzenacyclotetradecaphane-12-yl)-2- phenylacetamide | 630.3 | 631.4 |
| 362 | 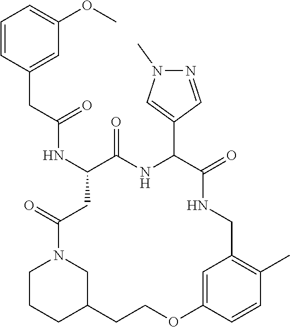 [SMILES: COc1cccc(CC(=O)N[C@H]2CC(=O)N3CCCC(CCOc4ccc(C)c(c4)CNC(=O)C(c4cnn(C)c4)NC2=O)C3)c1] | 2-(3-methoxyphenyl)-N-((12S)-5⁴- methyl-9-(1-methyl-1H-pyrazol-4-yl)- 8,11,14-trioxo-4-oxa-7,10-diaza-1(3,1)- piperidina-5(1,3)- benzenacyclotetradecaphane-12- yl)acetamide | 630.3 | 631.3 |

### Example A-37: Preparation of Macrocyclic Compound 495

### 4-(3-(2-(3-(isocyanomethyl)-4-methylphenoxy)ethyl)piperidin-1-yl)-4-oxobutanoic (481)

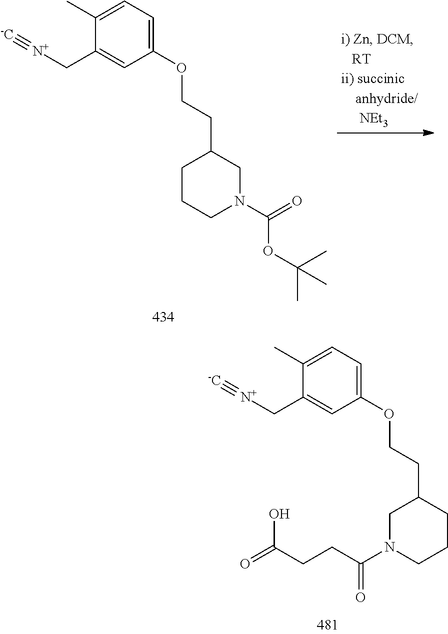

tert-butyl 3-(2-(3-(isocyanomethyl)-4-methylphenoxy)ethyl)piperidine-1-carboxylate (307 mg, 0.86 mmol, 1.0 eq.) was dissolved in DCM (10 mL) and zinc bromide (385 mg, 1.7 mmol, 2 eq.) was added to the solution. The reaction mixture was stirred for 72h to provide the free piperidine derivation in situ. MS (ES) C₁₆H₂₂N₂O requires: 258, found: 259 (M+H)+.

To the reaction mixture was added Et₃N (343 mg, 3.4 mmol, 4 eq.) and succinic anhydride (120 mg, 1.2 mmol, 1.4 eq.) and the reaction mixture was stirred for 2 h. The reaction mixture was diluted with DCM and H₂O. The aqueous layer was adjusted to pH 2-4 using a 0.1 M HCl solution before it was extracted 3× with DCM. The combined organic layers were dried over MgSO₄ and the solvents were removed under reduced pressure.

The crude product was purified by flash chromatography on silica gel (0-100% EtOAc/cHex, 0-100% MeOH/DCM) to yield the title compound 481 (50 mg, 0.14 mmol, 16%). Compound 481 is very unstable and hydrolyses easily to the corresponding formamide.

MS (ES) C₂₀H₂₆N₂O₄ requires: 358, found: 359 (M+H)⁺.

### 5⁴-methyl-9-phenethyl-4-oxa-7,10-diaza-1(3,1)-piperidina-5(1,3)-benzenacyclotetradecaphane-8,11,14-trione (495)

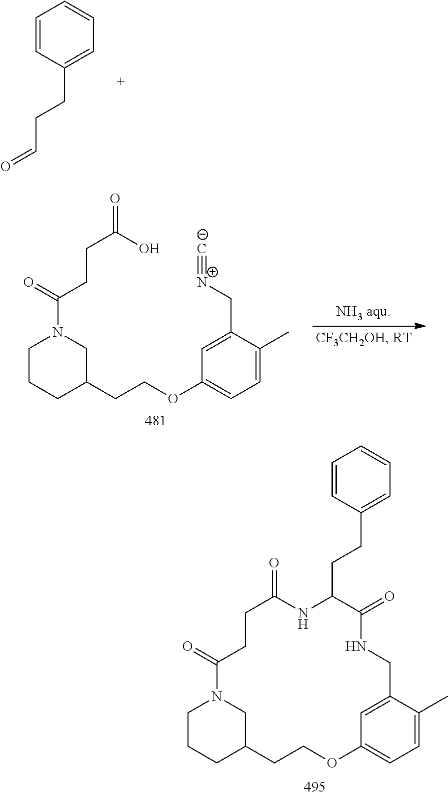 [SMILES: O=CCCc1ccccc1 ; [C-]#[N+]Cc1cc(OCCC2CCCN(C(=O)CCC(=O)O)C2)ccc1C ; Cc1ccc2cc1CNC(=O)C(CCc1ccccc1)NC(=O)CCC(=O)N1CCCC(CCO2)C1]

4-(3-(2-(3-(isocyanomethyl)-4-methylphenoxy)ethyl)piperidin-1-yl)-4-oxobutanoic acid (50 mg, 0.14 mmol, 1.0 eq.) was dissolved in 2,2,2-trifluoroethanol (1 mL), a solution of 3-phenylpropanal (52 mg, 0.40 mmol, 2.8 eq.) in 2,2,2-trifluoroethanol (1 mL), and aq. NH₃ (32%) (153 mg, 1.4 mmol, 10.0 eq.) were added to the solution. The reaction mixture was stirred for 10 min at RT.

The reaction mixture was diluted with DCM and NaHCO₃ and the aqueous layer was extracted 3× with DCM before the combined organic layers were dried over MgSO₄ and the solvents were removed in vacuo.

The crude product was purified by flash chromatography on silica gel (0-100% EtOAc/cHex, 0-100% MeOH/DCM) and by a subsequent reversed-phase RP-HPLC (column: C18), using H₂O (0.1% TFA) and ACN (0.1% TFA) as eluents. The desired fractions were lyophilized to yield the compound 495 (30 mg, 0.06 mmol, 44%) as a white solid. MS (ES) C₂₉H₃₇N₃O₄ requires: 491, found: 492 (M+H)⁺.

¹H NMR (400 MHz, d₆-DMSO, 300K) δ 8.42-7.70 (m, 2H), 7.33-7.24 (m, 2H), 7.24-7.13 (m, 3H), 7.06-7.00 (m, 1H), 6.99-6.66 (m, 2H), 4.77-3.72 (m, 6H), 3.10-2.54 (m, 4H), 2.54-2.26 (m, 3H), 2.20-2.12 (m, 3H), 2.10-0.97 (m, 11H).

### Example A-38: Preparation of Macrocyclic Compounds 503, 505, and 509-513

### tert-butyl (2S)-2-(((benzyloxy)carbonyl)amino)-4-(3-(2-(3-(isocyanomethyl)-4-methylphenoxy)ethyl)piperidin-1-yl)-4-oxobutanoate (515)

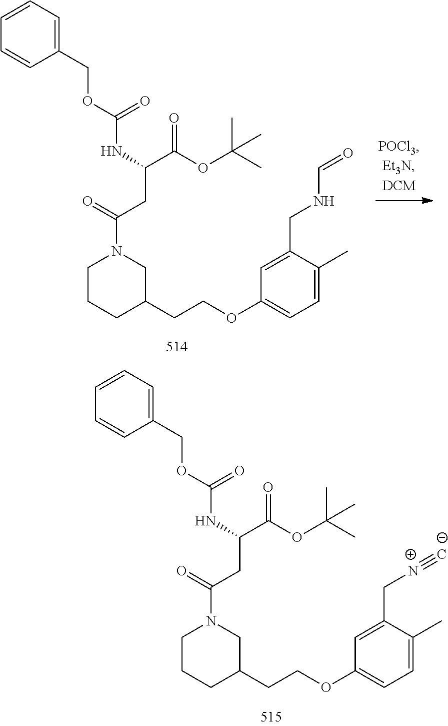 [SMILES: Cc1ccc(OCCC2CCCN(C(=O)C[C@H](NC(=O)OCc3ccccc3)C(=O)OC(C)(C)C)C2)cc1CNC=O ; [C-]#[N+]Cc1cc(OCCC2CCCN(C(=O)C[C@H](NC(=O)OCc3ccccc3)C(=O)OC(C)(C)C)C2)ccc1C]

tert-butyl (2S)-2-(((benzyloxy)carbonyl)amino)-4-(3-(2-(3-(formamidomethyl)-4-methylphenoxy)ethyl)piperidin-1-yl)-4-oxobutanoate (100 mg, 0.172 mmol, 1.0 eq.) was dissolved in DCM (10 mL) and Et₃N (104 mg, 1.0 mmol, 6 eq.) was added to the solution. A solution of POCl₃ (53 mg, 0.34 mmol, 2 eq.) in DCM (1 mL) was added dropwise to the reaction mixture at 0° C. After 1h still 20% of starting material was observed. An additional solution of POCl₃ (26 mg, 0.172 mmol, 1 eq.) in DCM (0.5 mL) was added dropwise to the reaction mixture at 0° C. The reaction was kept for further 30 min.

The reaction mixture was diluted with DCM and NaHCO₃ and the aqueous layer was extracted 3× with DCM. The combined organic layers were dried over MgSO₄ and the solvents were removed under reduced pressure.

The crude product was purified by flash chromatography on silica gel (0-100% EtOAc/cHex) to yield the title compound 515 (95 mg, 0.168 mmol, 98%). MS (ES) C₃₂H₄₁N₃O₆ requires: 563, found: 564 (M+H)⁺.

### (2S)-2-(((benzyloxy)carbonyl)amino)-4-(3-(2-(3-(isocyanomethyl)-4-methylphenoxy)ethyl)piperidin-1-yl)-4-oxobutanoic acid (516)

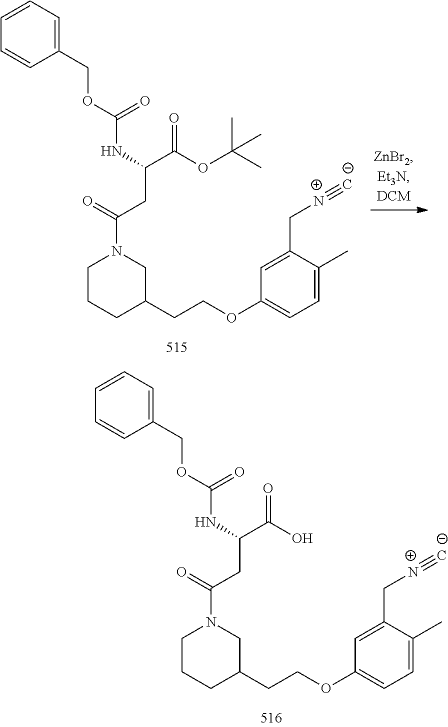 [SMILES: [C-]#[N+]Cc1cc(OCCC2CCCN(C(=O)C[C@H](NC(=O)OCc3ccccc3)C(=O)OC(C)(C)C)C2)ccc1C ; [C-]#[N+]Cc1cc(OCCC2CCCN(C(=O)C[C@H](NC(=O)OCc3ccccc3)C(=O)O)C2)ccc1C]

To a solution of tert-butyl (2S)-2-(((benzyloxy)carbonyl)amino)-4-(3-(2-(3-(isocyanomethyl)-4-methylphenoxy)ethyl)piperidin-1-yl)-4-oxobutanoate (1 g, 1.77 mmol, 1.0 eq.) in DCM (40 mL) was added Et₃N (358 mg, 3.54 mmol, 2 eq.) and zinc bromide (2 g, 8.9 mmol, 5 eq.). The reaction mixture was stirred for 16 h.

The reaction mixture was purified by reversed-phase chromatography (column: C18), using H₂O and ACN as eluents. The desired fractions were directly lyophilized to yield title compound 516 (672 mg, 1.32 mmol, 75%) as a white solid. MS (ES) C₂₈H₃₃N₃O₆ requires: 507, found: 508 (M+H)⁺.

### benzyl ((12S)-9-(2-(1,5-naphthyridin-3-yl)ethyl)-5⁴-methyl-8,11,14-trioxo-4-oxa-7,10-diaza-1(3,1)-piperidina-5(1,3)-benzenacyclotetradecaphane-12-yl)carbamate (511)

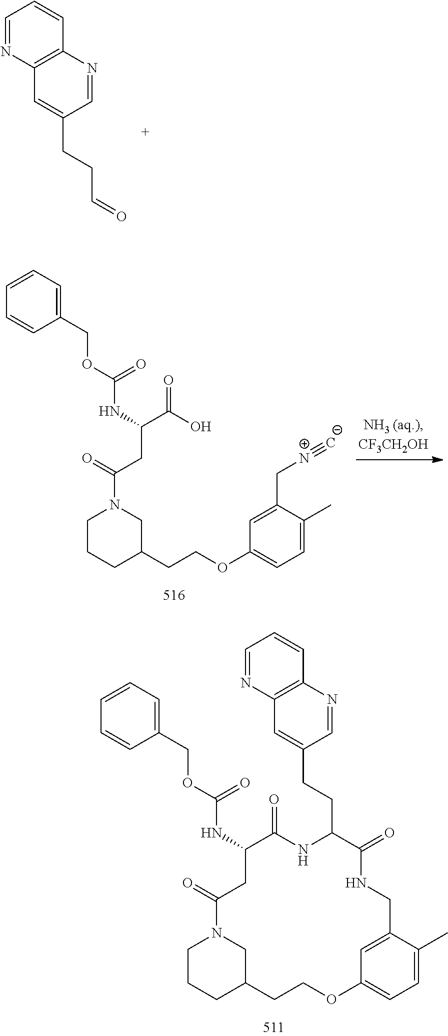 [SMILES: O=CCCc1cnc2cccnc2c1 ; [C-]#[N+]Cc1cc(OCCC2CCCN(C(=O)C[C@H](NC(=O)OCc3ccccc3)C(=O)O)C2)ccc1C ; Cc1ccc2cc1CNC(=O)C(CCc1cnc3cccnc3c1)NC(=O)[C@@H](NC(=O)OCc1ccccc1)CC(=O)N1CCCC(CCO2)C1]

(2S)-2-(((benzyloxy)carbonyl)amino)-4-(3-(2-(3-(isocyanomethyl)-4-methylphenoxy)ethyl)piperidin-1-yl)-4-oxobutanoic acid (77 mg, 0.15 mmol, 1.0 eq.) was dissolved in 2,2,2-trifluoroethanol (4 mL), 3-(1,5-naphthyridin-3-yl)propanal (39 mg, 0.21 mmol, 1.4 eq.) and aq. NH₃ (32%) (550 mg, 0.46 mmol, 3.0 eq.) were added to the solution. The reaction mixture was stirred for 12 h at RT.

The reaction mixture was diluted with DCM and NaHCO₃ and the aqueous layer was extracted 3× with DCM before the combined organic layers were dried over MgSO₄ and the solvents were removed in vacuo.

The crude product was purified by flash chromatography on silica gel (0-100% EtOAc/cHex, 0-100% MeOH/DCM) and by a subsequent reversed-phase RP-HPLC (column: C18), using H₂O (0.1% TFA) and ACN (0.1% TFA) as eluents to yield the title compound 511 (35 mg, 0.04 mmol, 29%) as a yellowish solid (TFA salt). MS (ES) C₃₉H₄₄N₆O₆ requires: 692, found: 693 (M+H)⁺.

¹H NMR (400 MHz, d₆-DMSO, 300K) δ 9.14-8.71 (m, 2H), 8.63-7.56 (m, 5H), 7.52-6.96 (m, 7H), 6.98-6.54 (m, 2H), 5.22-4.87 (m, 2H), 4.68-3.62 (8H), 3.13-2.58 (m, 6H), 2.22-1.88 (m, 4H), 1.81-1.02 (m, 8H).

The examples in the following table were prepared according to the procedure described for compound 511.

TABLE A-41

| Cpd | | | Exact | [M + H]⁺ |
|---|---|---|---|---|
| no | structure | name | mass | found |
| 511 | 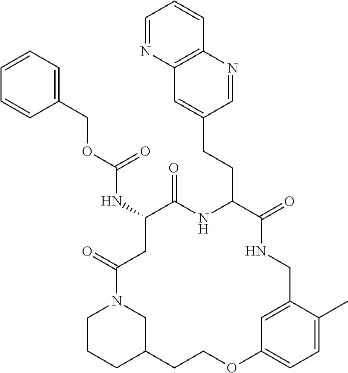 [SMILES: Cc1ccc2cc1CNC(=O)C(CCc1cnc3cccnc3c1)NC(=O)[C@@H](NC(=O)OCc1ccccc1)CC(=O)N1CCCC(CCO2)C1] | benzyl ((12S)-9-(2-(1,5- naphthyridin-3-yl)ethyl)-5⁴-methyl- 8,11,14-trioxo-4-oxa-7,10-diaza- 1(3,1)-piperidina-5(1,3)- benzenacyclotetradecaphane-12- yl)carbamate | 692 | 693 |
| 503 | 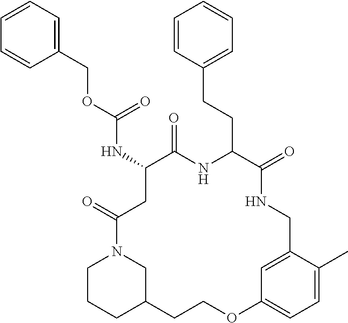 [SMILES: Cc1ccc2cc1CNC(=O)C(CCc1ccccc1)NC(=O)[C@@H](NC(=O)OCc1ccccc1)CC(=O)N1CCCC(CCO2)C1] | benzyl ((12S)-9-phenethyl-5⁴- methyl-8,11,14-trioxo-4-oxa-7,10- diaza-1(3,1)-piperidina-5(1,3)- benzenacyclotetradecaphane-12- yl)carbamate | 640 | 641 |
| 505 | 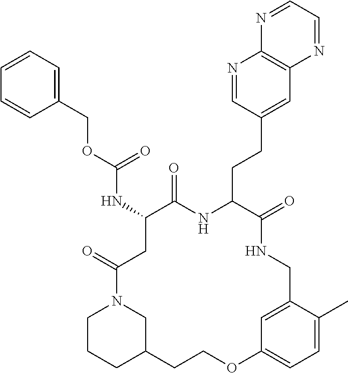 [SMILES: Cc1ccc2cc1CNC(=O)C(CCc1cnc3nccnc3c1)NC(=O)[C@@H](NC(=O)OCc1ccccc1)CC(=O)N1CCCC(CCO2)C1] | benzyl ((12S)-5⁴-methyl-8,11,14- trioxo-9-(2-(pyrido[2,3-b]pyrazin-7- yl)ethyl)-4-oxa-7,10-diaza-1(3,1)- piperidina-5(1,3)- benzenacyclotetradecaphane-12- yl)carbamate | 693 | 694 |
| 509 | 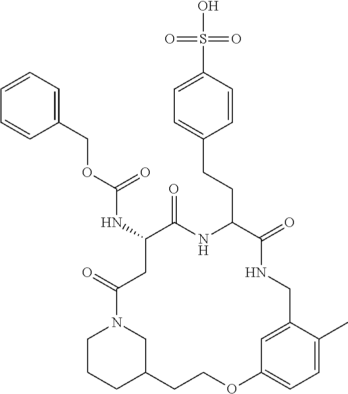 [SMILES: Cc1ccc2cc1CNC(=O)C(CCc1ccc(S(=O)(=O)O)cc1)NC(=O)[C@@H](NC(=O)OCc1ccccc1)CC(=O)N1CCCC(CCO2)C1] | 4-(2-((12S)-12- (((benzyloxy)carbonyl)amino)-5⁴- methyl-8,11,14-trioxo-4-oxa-7,10- diaza-1(3,1)-piperidina-5(1,3)- benzenacyclotetradecaphane-9- yl)ethyl)benzenesulfonic acid | 720 | 721 |
| 510 | 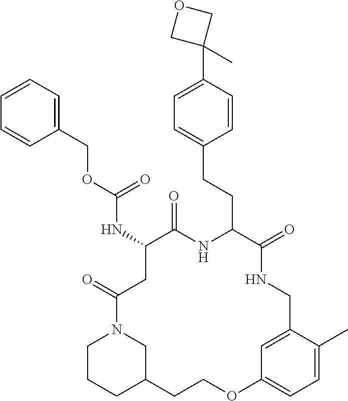 [SMILES: Cc1ccc2cc1CNC(=O)C(CCc1ccc(C3(C)COC3)cc1)NC(=O)[C@@H](NC(=O)OCc1ccccc1)CC(=O)N1CCCC(CCO2)C1] | benzyl ((12S)-5⁴-methyl-9-(4-(3- methyloxetan-3-yl)phenethyl)- 8,11,14-trioxo-4-oxa-7,10-diaza- 1(3,1)-piperidina-5(1,3)- benzenacyclotetradecaphane-12- yl)carbamate | 710 | 711 |
| 512 | 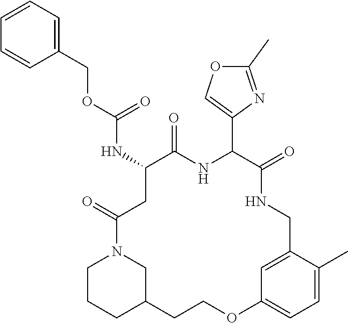 [SMILES: Cc1nc(C2NC(=O)[C@@H](NC(=O)OCc3ccccc3)CC(=O)N3CCCC(CCOc4ccc(C)c(c4)CNC2=O)C3)co1] | benzyl ((12S)-54-methyl-9-(2- methyloxazol-4-yl)-8, 11,14-trioxo- 4-oxa-7,10-diaza-1(3,1)-piperidina- 5(1,3)- benzenacyclotetradecaphane-12- yl)carbamate | 617 | 618 |
| 513 | 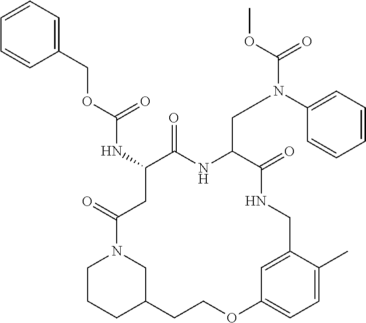 [SMILES: COC(=O)N(CC1NC(=O)[C@@H](NC(=O)OCc2ccccc2)CC(=O)N2CCCC(CCOc3ccc(C)c(c3)CNC1=O)C2)c1ccccc1] | methyl (((12R)-12- (((benzyloxy)carbonyl)amino)-54- methyl-8,11,14-trioxo-4-oxa-7,10- diaza-1(3,1)-piperidina-5(1,3)- benzenacyclotetradecaphane-9- yl)methyl)(phenyl)carbamate | 699 | 700 |

### Example A-39: Preparation of Macrocyclic Compounds 565-568

### N-((12S)-9-(1-benzyl-1H-pyrazol-4-yl)-5⁴-methyl-8,11,14-trioxo-4-oxa-7,10-diaza-1(3,1)-piperidina-5(1,3)-benzenacyclotetradecaphane-12-yl)acetamide (565)

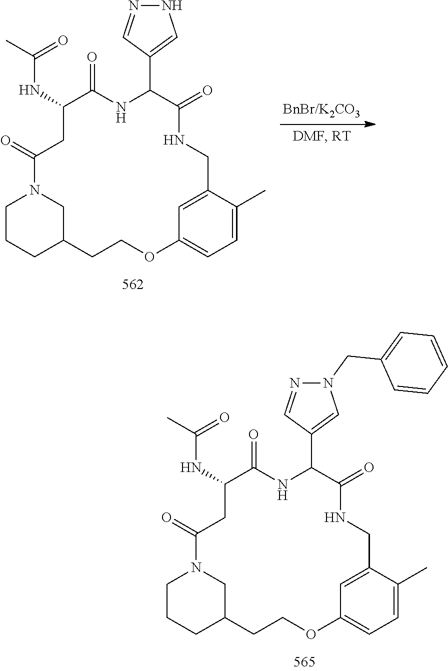 [SMILES: CC(=O)N[C@H]1CC(=O)N2CCCC(CCOc3ccc(C)c(c3)CNC(=O)C(c3cn[nH]c3)NC1=O)C2 ; CC(=O)N[C@H]1CC(=O)N2CCCC(CCOc3ccc(C)c(c3)CNC(=O)C(c3cnn(Cc4ccccc4)c3)NC1=O)C2]

N-((12S)-5⁴-methyl-8,11,14-trioxo-9-(1H-pyrazol-4-yl)-4-oxa-7,10-diaza-1(3,1)-piperidina-5(1,3)-benzenacyclotetradecaphane-12-yl)acetamide 562 (50 mg, 0.098 mmol, 1.0 eq.) was dissolved in DMF (2 ml), potassium carbonate (41 mg, 0.29 mmol, 3.0 eq.), and benzyl bromide (21 mg, 0.12 mmol, 1.2 eq.) were added to the solution at RT and the reaction mixture was stirred for 12h.

The reaction mixture was diluted with EtOAc and NaHCO₃ and the aqueous layer was extracted 3× with EtOAc. The combined organic layers were dried over MgSO₄ and the solvent was removed in vacuo.

The crude product was purified by flash chromatography on silica gel (0-100% EtOAc/cHex, 0-100% MeOH/DCM) and by a subsequent reversed-phase RP-HPLC (column: C18), using H₂O (0.1% TFA) and ACN (0.1% TFA) as eluents. The desired fractions were lyophilized to yield title compound 565 (23.4 mg, 0.033 mmol, 34%) as a white solid (TFA salt). MS (ES) C₃₃H₄ON₆O₅ requires: 600, found: 601 (M+H)⁺.

¹H NMR (400 MHz, d₆-DMSO, 300K) δ 8.69-8.34 (m, 1H), 8.27-7.65 (m, 3H), 7.54-7.20 (m, 6H), 7.09-7.02 (m, 1H), 6.97-6.66 (m, 2H), 5.57-5.21 (m, 3H), 4.77-4.04 (m, 6H), 3.21-2.53 (m, 3H), 2.22-2.02 (m, 4H), 1.95-1.16 (m, 11H).

Examples in the following table were prepared similar to the procedure described for 565.

TABLE A-42

| Cpd | | | | |
|---|---|---|---|---|
| No | Structure | name | M | [M + H]⁺ |
| 566 | 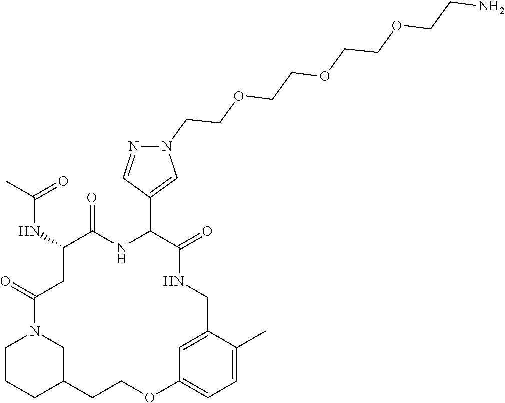 [SMILES: CC(=O)N[C@H]1CC(=O)N2CCCC(CCOc3ccc(C)c(c3)CNC(=O)C(c3cnn(CCOCCOCCOCCN)c3)NC1=O)C2] | N-((12S)-9-(1-(2-(2-(2-(2- aminoethoxy)ethoxy)ethoxy) ethyl)-1H-pyrazol-4-yl)-5⁴-methyl- 8,11,14-trioxo-4-oxa-7,10-diaza- 1(3,1)-piperidina-5(1,3)- benzenacyclotetradecaphane-12- yl)acetamide | 685 | 686 |
| 567 | 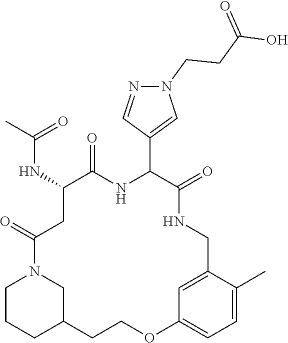 [SMILES: CC(=O)N[C@H]1CC(=O)N2CCCC(CCOc3ccc(C)c(c3)CNC(=O)C(c3cnn(CCC(=O)O)c3)NC1=O)C2] | 3-(4-((12S)-12-acetamido-5⁴- methyl-8,11,14-trioxo-4-oxa-7,10- diaza-1(3,1)-piperidina-5(1,3)- benzenacyclotetradecaphane-9- yl)-1H-pyrazol-1-yl)propanoic acid | 582 | 583 |
| 568 | 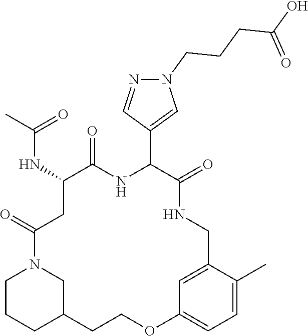 [SMILES: CC(=O)N[C@H]1CC(=O)N2CCCC(CCOc3ccc(C)c(c3)CNC(=O)C(c3cnn(CCCC(=O)O)c3)NC1=O)C2] | 4-(4-((12S)-12-acetamido-5⁴- methyl-8,11,14-trioxo-4-oxa-7,10- diaza-1(3,1)-piperidina-5(1,3)- benzenacyclotetradecaphane-9- yl)-1H-pyrazol-1-yl)butanoic acid | 596 | 597 |

In order to obtain the examples 566, 567 and 568 subsequent standard acidic BOC (or tert-butyl)-deprotection according to general procedure D was performed.

### Example A-40: Preparation of Macrocyclic Compounds 570-572

### N-((12S)-5⁴-methyl-8,11,14-trioxo-9-(1-(3-(tritylthio)propyl)-1H-pyrazol-4-yl)-4-oxa-7,10-diaza-1(3,1)-piperidina-5(1,3)-benzenacyclotetradecaphane-12-yl)acetamide (569)

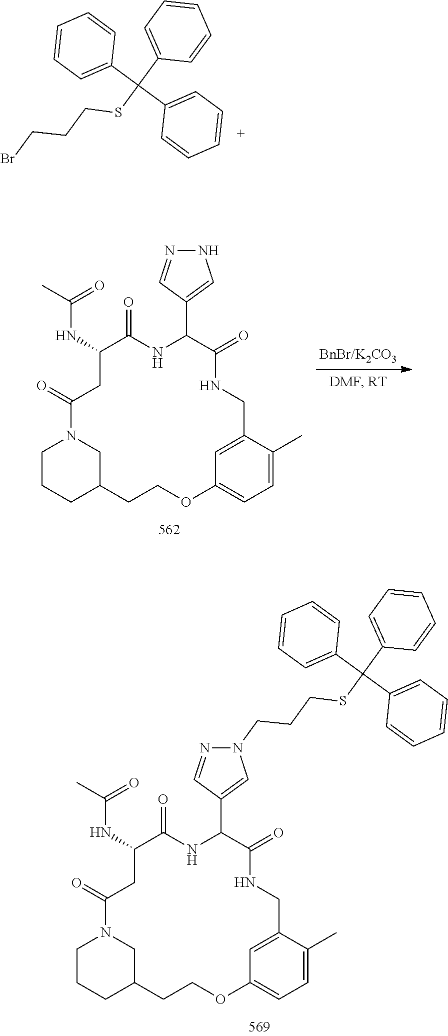 [SMILES: BrCCCSC(c1ccccc1)(c1ccccc1)c1ccccc1 ; CC(=O)N[C@H]1CC(=O)N2CCCC(CCOc3ccc(C)c(c3)CNC(=O)C(c3cn[nH]c3)NC1=O)C2 ; CC(=O)N[C@H]1CC(=O)N2CCCC(CCOc3ccc(C)c(c3)CNC(=O)C(c3cnn(CCCSC(c4ccccc4)(c4ccccc4)c4ccccc4)c3)NC1=O)C2]

N-((12S)-5⁴-methyl-8,11,14-trioxo-9-(1H-pyrazol-4-yl)-4-oxa-7,10-diaza-1(3,1)-piperidina-5(1,3)-benzenacyclotetradecaphane-12-yl)acetamide 562 (100 mg, 0.20 mmol, 1.0 eq.) was dissolved in DMF (5 mL), potassium carbonate (81 mg, 0.59 mmol, 3.0 eq.), and (3-bromopropyl)(trityl)sulfane (187 mg, 0.47 mmol, 2.4 eq.) were added to the solution at RT and the reaction mixture was heated to 80° C. and stirred for 18h. The reaction mixture was diluted with DCM and NaHCO₃ and the aqueous layer was extracted 3× with DCM. The combined organic layers were dried over MgSO₄ and the solvent was removed in vacuo. The crude product was purified by flash chromatography on silica gel (0-100% EtOAc/cHex, 0-100% MeOH/DCM) and led to the desired title compound 569 (35.2 mg, 0.043 mmol, 22%). MS (ES) C₄₈H₅₄N₆O₅S requires: 826, found: 827 (M+H)⁺.

### N-((12S)-9-(1-(3-mercaptopropyl)-1H-pyrazol-4-yl)-5⁴-methyl-8,11,14-trioxo-4-oxa-7,10-diaza-1(3,1)-piperidina-5(1,3)-benzenacyclotetradecaphane-12-yl)acetamide (570)

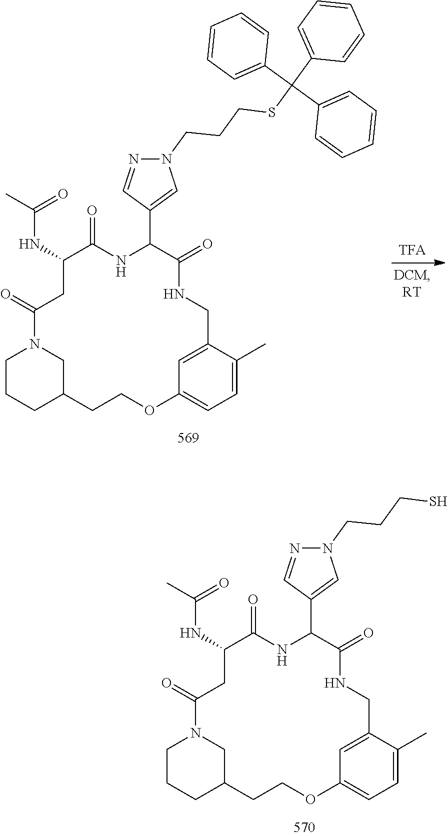 [SMILES: CC(=O)N[C@H]1CC(=O)N2CCCC(CCOc3ccc(C)c(c3)CNC(=O)C(c3cnn(CCCSC(c4ccccc4)(c4ccccc4)c4ccccc4)c3)NC1=O)C2 ; CC(=O)N[C@H]1CC(=O)N2CCCC(CCOc3ccc(C)c(c3)CNC(=O)C(c3cnn(CCCS)c3)NC1=O)C2]

N-((12S)-5⁴-methyl-8,11,14-trioxo-9-(1-(3-(tritylthio)propyl)-1H-pyrazol-4-yl)-4-oxa-7,10-diaza-1(3,1)-piperidina-5(1,3)-benzenacyclotetradecaphane-12-yl)acetamide (35.2 mg, 0.043 mmol, 1.0 eq.) was dissolved in 25% TFA/DCM (3 mL) and stirred for 30 min at RT. Removing the solvents under reduced pressure led to the title compound 570 (30 mg, 0.043 mmol, quant.) as TFA salt. MS (ES) C₂₉H₄₀N₆O₅S requires: 584, found: 585 (M+H)⁺.

### 3-(4-((12S)-12-acetamido-5⁴-methyl-8,11,14-trioxo-4-oxa-7,10-diaza-1(3,1)-piperidina-5(1,3)-benzenacyclotetradecaphane-9-yl)-1H-pyrazol-1-yl)propane-1-sulfonic acid (571)

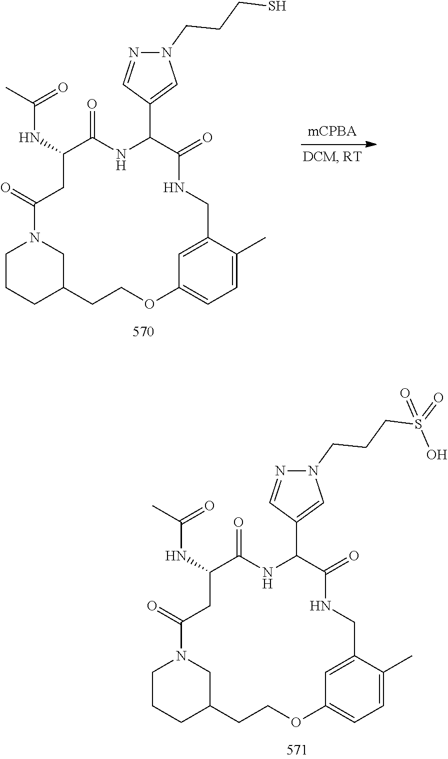 [SMILES: CC(=O)N[C@H]1CC(=O)N2CCCC(CCOc3ccc(C)c(c3)CNC(=O)C(c3cnn(CCCS)c3)NC1=O)C2 ; CC(=O)N[C@H]1CC(=O)N2CCCC(CCOc3ccc(C)c(c3)CNC(=O)C(c3cnn(CCCS(=O)(=O)O)c3)NC1=O)C2]

N-((12S)-9-(1-(3-mercaptopropyl)-1H-pyrazol-4-yl)-5⁴-methyl-8,11,14-trioxo-4-oxa-7,10-diaza-1(3,1)-piperidina-5(1,3)-benzenacyclotetradecaphane-12-yl)acetamide 570 (25 mg, 0.043 mmol, 1.0 eq.) was dissolved in DCM (5 mL) and 3-chlorobenzoperoxoic acid (45 mg, 0.26 mmol, 6.0 eq.) was added to the solution at 0° C. and the reaction mixture was stirred for 12 h at RT. Drops of H₂O were added to the reaction and the solvents were removed in vacuo.

The crude product was purified by reversed-phase RP-HPLC (column: C18), using H₂O (0.1% TFA) and ACN (0.1% TFA) as eluents. The desired fractions were lyophilized to yield the title compound 711 (7 mg, 0.009 mmol, 22%) as a yellowish solid (TFA salt). MS (ES) C₂₉H₄₀N₆O₈S requires: 632, found: 633 (M+H)⁺.

### N-(4-((12S)-12-acetamido-5⁴-methyl-8,11,14-trioxo-4-oxa-7,10-diaza-1(3,1)-piperidina-5(1,3)-benzenacyclotetradecaphane-9-yl)-1H-pyrazol-1-yl)ethane-1-sulfonic acid (572)

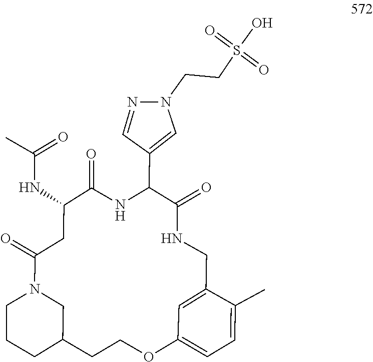 [SMILES: CC(=O)N[C@H]1CC(=O)N2CCCC(CCOc3ccc(C)c(c3)CNC(=O)C(c3cnn(CCS(=O)(=O)O)c3)NC1=O)C2]

Compound 2-(4-((12S)-12-acetamido-5⁴-methyl-8,11,14-trioxo-4-oxa-7,10-diaza-1(3,1)-piperidina-5(1,3)-benzenacyclotetradecaphane-9-yl)-1H-pyrazol-1-yl)ethane-1-sulfonic acid 571 was prepared in analogy to compound 570. MS (ES) C₂₉H₄₀N₆O₈S requires: 618, found: 619 (M+H)⁺.

### Preparation of Macrocyclic Compounds 573-741

### General Methods

¹H NMR spectra were recorded on Bruker 400 MHz and TMS was used as an internal standard.

LCMS was taken on a quadrupole Mass Spectrometer on Shimadzu LCMS 2010 (Shim-pack XR-ODS 3.0*30 mm 2.2 μm) operating in ES (+) ionization mode. Flow Rate: 0.8 mL/min, Acquire Time: 3 min, Wavelength: UV220, Oven Temp.: 50° C.

Prep-HPLC was performed at conditions: Column: YMC Triart (30*150 mm*7 um); Wavelength 220 nm; Mobile phase A: water (NH₃H₂O+NH₄HCO₃); B acetonitrile; Flow rate: 25 mL/min; Injection volume: 2 mL; Run time: 10 min; Equilibration: 9.0 min.

### General Methods

¹H NMR spectra were recorded on Bruker 400 MHz and TMS was used as an internal standard.

LCMS was taken on a quadrupole Mass Spectrometer on Shimadzu LCMS 2010 (Shim-pack XR-ODS 3.0*30 mm 2.2 μm) operating in ES (+) ionization mode. Flow Rate: 0.8 mL/min, Acquire Time: 3 min, Wavelength: UV220, Oven Temp.: 50° C.

Prep-HPLC was performed at conditions: Column: YMC Triart (30*150 mm*7 um); Wavelength 220 nm; Mobile phase A: water (NH₃H₂O+NH₄HCO₃); B acetonitrile; Flow rate: 25 mL/min; Injection volume: 2 mL; Run time: 10 min; Equilibration: 9.0 min.

### Example A-41: Preparation of Macrocyclic Compound 733

### 1. Preparation of Compound A41-2:

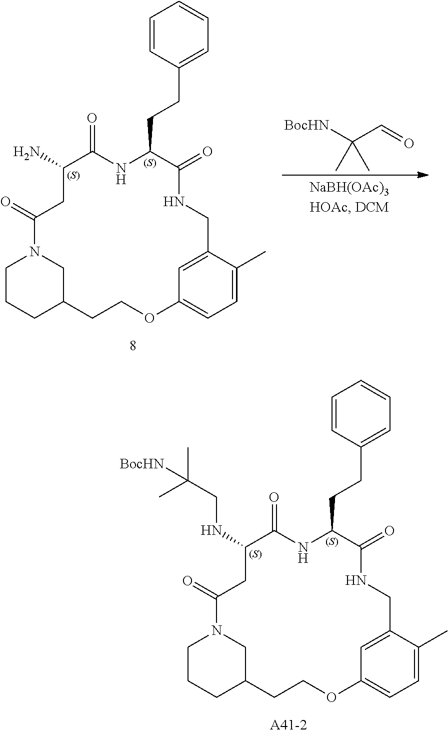

To a solution of 8 (300 mg, 0.552 mmol, 1 eq, HCl) in DCM (18 mL) was added DIEA (142.78 mg, 1.10 mmol, 0.192 mL, 2 eq), tert-butyl N-(1,1-dimethyl-2-oxo-ethyl)carbamate (310.28 mg, 1.66 mmol, 3 eq) and AcOH (66.34 mg, 1.10 mmol, 0.063 mL, 2 eq), the reaction was stirred at 20° C. for 2 hr to give a white mixture. Then added NaBH(OAc)₃ (234.15 mg, 1.10 mmol, 2 eq) to the mixture and it was stirred at 20° C. for 17 hr to give a white mixture. LCMS showed a desired mass. The reaction mixture was diluted with H₂O (50 mL) and extracted with EA (40 mL×2). The combined organic layers were washed with brine (80 mL), dried over Na₂SO₄, filtered and concentrated under reduced pressure to give a residue. The A41-2 (900 mg, crude) as yellow oil was used to the next step without purification.

### 2. Preparation of Compound A41-3:

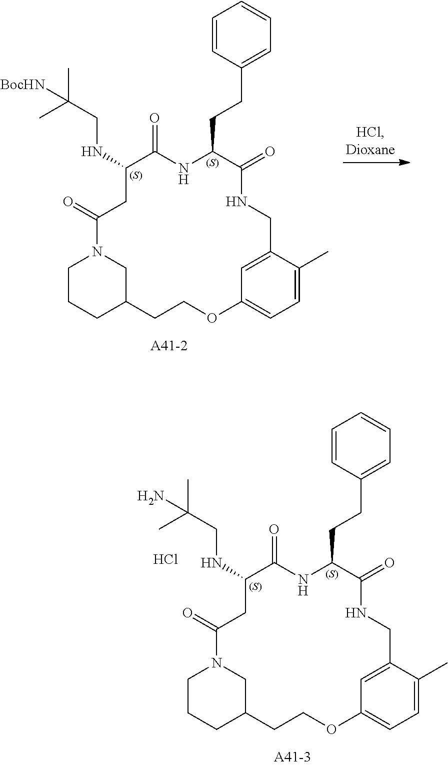

To a solution of A41-2 (900 mg, 1.33 mmol, 1 eq) in dioxane (5 mL) was added HCl/Dioxane (4 M, 3.32 mL, 10 eq). The mixture was stirred at 20° C. for 1 hr to give a light yellow solution. LCMS showed A41-2 was remained. Added 3 mL HCl/dioxane to the reaction and it was stirred for 1 hr to give a light yellow solution. LCMS showed A41-2 was consumed up. The reaction solution was concentrated under reduced pressure to give a residue. The A41-3 (800 mg, 1.30 mmol, 98.10% yield, HCl) as yellow gum was used to the next step without purification.

### 3. Preparation of Compound 733:

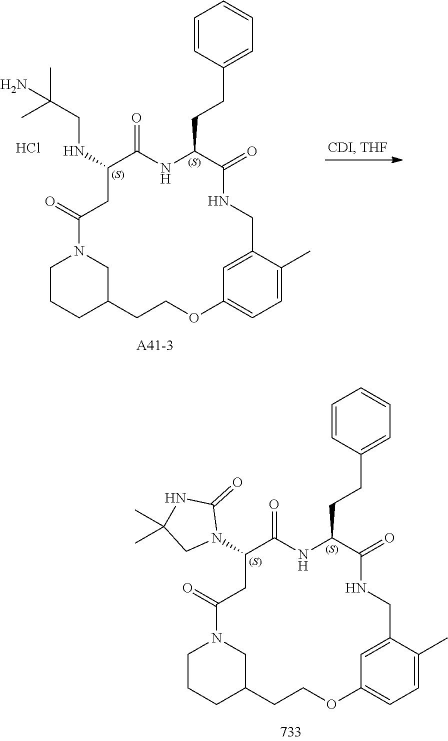

To a solution of A41-3 (700 mg, 1.14 mmol, 1 eq, HCl) in THF (15 mL) was added CDI (1.11 g, 6.84 mmol, 6 eq). The mixture was stirred at 70° C. for 6 hr to give a yellow solution. LCMS showed A41-3 was consumed up. The reaction mixture was diluted with H₂O (50 mL) and extracted with EA (40 mL×3). The combined organic layers were washed with brine (80 mL×2), dried over Na₂SO₄, filtered and concentrated under reduced pressure to give a residue. The residue was purified by prep-HPLC (basic condition, column: YMC Triart 30*150 mm*7 um; mobile phase: [water (NH₃H₂O+NH₄HCO₃)-ACN]; B %: 43%-63%, 9 min) to give 733 (154.1 mg, 0.255 mmol, 22.40% yield) as white solid.

¹H NMR (DMSO-d₆, 400 MHz): δ=7.59-8.55 (m, 2H), 7.08-7.34 (m, 5H), 6.83-7.06 (m, 2H), 6.55 (br s, 1H), 6.42-6.77 (m, 1H), 4.47-4.74 (m, 2H), 3.60-4.44 (m, 7H), 2.79-3.31 (m, 4H), 2.59-2.73 (m, 2H), 2.29-2.47 (m, 1H), 2.07-2.22 (m, 3H), 1.88-2.02 (m, 1H), 1.27-1.81 (m, 7H), 1.10-1.20 (m, 1H), 1.10-1.20 (m, 1H), 1.06-1.23 ppm (m, 6H).

LCMS: 100.00%, MS (ESI): m/z 604.3 [M+H]⁺.

### Example A-42: Preparation of Macrocyclic Compound 717

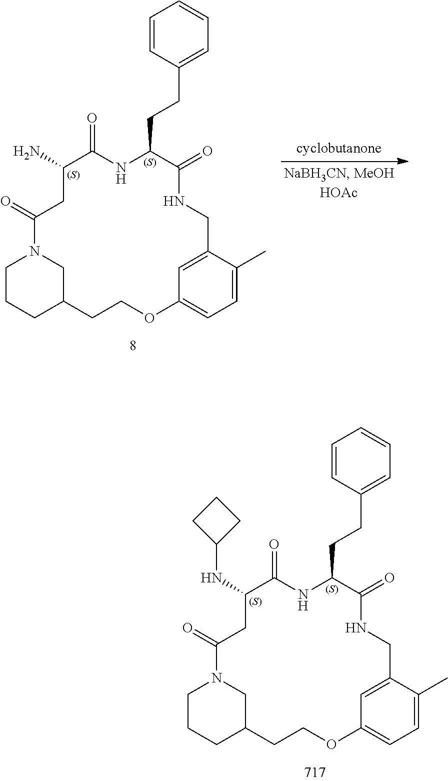

To a solution of 8 (0.15 g, 0.276 mmol, 1 eq, HCl), DIEA (71 mg, 0.552 mmol, 2 eq) in DCM (6 mL) was added cyclobutanone (39 mg, 0.552 mmol, 2 eq), AcOH (33 mg, 0.552 mmol, 2 eq). The reaction was stirred at 20° C. for 1.5 h to give a yellow mixture. NaBH(OAc)₃ (117 mg, 0.552 mmol, 2 eq) was added. The reaction was stirred at 20° C. for 17 h to give a yellow mixture. LCMS showed the reaction was completed. The mixture was partitioned between EA (60 mL) and H₂O (40 mL). The organic layer was washed with H₂O (40 mL), brine (40 mL), dried over anhydrous Na₂SO₄, filtered and concentrated under reduced pressure to give a white powder. The crude product was purified by prep-HPLC(column: 2_Phenomenex Gemini C18 75*40 mm*3 um; mobile phase: [water(NH₃H₂O+NH₄HCO₃)-ACN];B %: 46%-76%, 7.8 min). The eluent was lyophilized to give 717 (32.9 mg, 21% yield, 100% purity) as a white powder.

¹H NMR (400 MHz, DMSO-d₆) δ ppm 1.16-2.36 (m, 20H), 2.58-3.00 (m, 4H), 3.09-3.29 (m, 2H), 3.49-4.76 (m, 8H), 6.60-7.08 (m, 3H), 7.12-7.36 (m, 5H), 7.84-8.43 (m, 2H)

LCMS: 100%, MS (ESI): m/z 561.3 [M+H]⁺.

### Example A-43: Preparation of Macrocyclic Compound 711

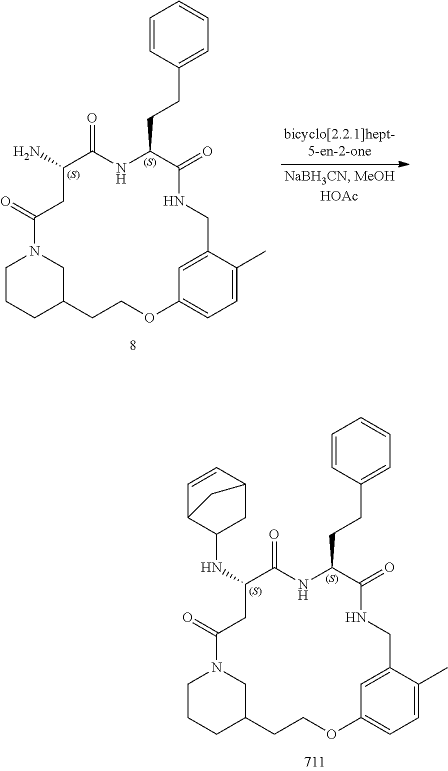

To a solution of 8 (0.15 g, 0.276 mmol, 1 eq, HCl), DIEA (71 mg, 0.552 mmol, 2 eq) in DCM (6 mL) was added bicyclo[2.2.1]hept-5-en-2-one (60 mg, 0.552 mmol, 2 eq), AcOH (33 mg, 0.552 mmol, 2 eq). The reaction was stirred at 20° C. for 1.5 h to give a yellow mixture. NaBH(OAc)₃ (117 mg, 0.552 mmol, 2 eq) was added. The reaction was stirred at 20° C. for 17 h to give a yellow mixture. LCMS showed the starting material was not consumed completely. NaBH(OAc)₃ (117 mg, 0.552 mmol, 2 eq) was added. The reaction was stirred at 20° C. for 8 h to give a yellow mixture. LCMS showed the starting material was consumed completely. The mixture was partitioned between EA (60 mL) and H₂O (40 mL). The organic layer was washed with H₂O (40 mL), brine (40 mL), dried over anhydrous Na₂SO₄, filtered and concentrated under reduced pressure to give a white powder. The crude product was triturated with EtOH (2 mL). After filtration, the filter cake was dried under reduced pressure to give a white solid. MeCN (2 mL) and H₂O (2 mL) were added. The mixture was lyophilized to give 711 (78.1 mg, 47% yield) as a white powder.

¹H NMR (400 MHz, DMSO-d₆) δ ppm 0.96-2.37 (m, 18H), 2.55-3.31 (m, 8H), 3.59-4.81 (m, 8H), 5.78-6.28 (m, 2H), 6.54-7.38 (m, 8H), 7.84-8.65 (m, 2H)

LCMS: 100%, MS (ESI): m/z 599.3 [M+H]⁺.

### Example A-44: Preparation of Macrocyclic Compound 653

### 1. Preparation of A42-2:

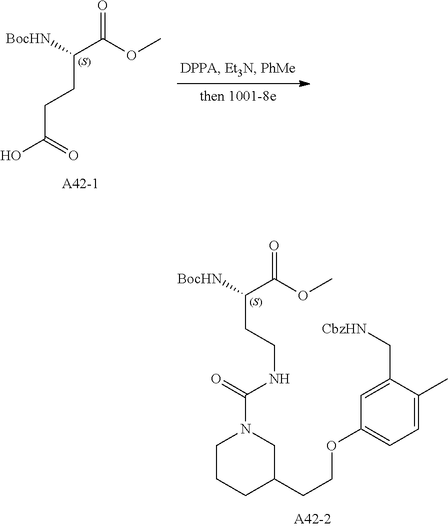

To a solution of A42-1(0.3 g, 1.15 mmol, 1 eq), DPPA (diphenylphosphoryl azide) (443 mg, 1.61 mmol, 1.4 eq) in toluene (11 mL) was Et₃N (233 mg, 2.30 mmol, 2 eq). The reaction was stirred at 80° C. for 1 h to give a colorless solution. 1001-8e (578 mg, 1.38 mmol, 1.2 eq, HCl), Et₃N (233 mg, 2.30 mmol, 2 eq) was added.

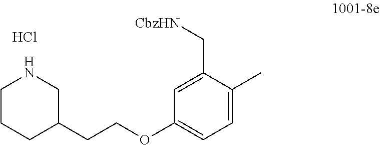 [SMILES: Cc1ccc(OCCC2CCCNC2)cc1CNC(=O)OCc1ccccc1 ; Cl]

The reaction was stirred at 20° C. for 17 h to give a yellow mixture. LCMS showed desired MS was detected. The solution was partitioned between EA (60 mL) and H₂O (20 mL). The organic layer was washed with sat. NaHCO₃ (20 mL×2), brine (20 mL), dried over anhydrous Na₂SO₄, concentrated under reduced pressure to give a yellow oil. The yellow oil was purified by combi flash (EA/PE=0/1 to 1/1) to give A42-2 (0.41 g, 55% yield) as yellow oil.

### 2. Preparation of A42-3:

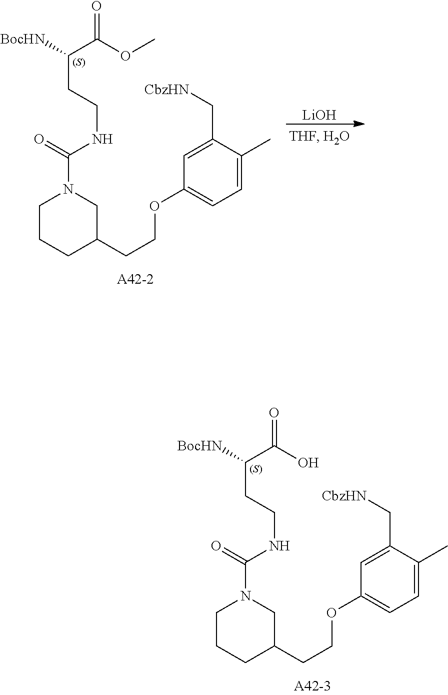

To a solution of A42-2 (0.41 g, 0.640 mmol, 1 eq) in THF (5 mL) was added LiOH·H₂O (31 mg, 0.736 mmol, 1.15 eq) in H₂O (1 mL). The reaction was stirred at 20° C. for 1 h to give a yellow mixture. LCMS showed the starting material was not consumed completely. A solution of LiOH H₂O (30 mg) in H₂O (1 mL) was added. The reaction was stirred at 20° C. for 1 h to give a yellow mixture. LCMS showed the reaction was completed. H₂O (15 mL) was added. Then the aqueous layer was acidified to pH=4 by addition of 1 M HCl. The aqueous layer was extracted with DCM (20 mL×2). The combined organic layer was washed with brine (10 mL), dried over anhydrous Na₂SO₄, filtered and concentrated in vacuum to give A42-3 (0.352 g, 88% yield) as yellow gum.

### 3. Preparation of A42-4:

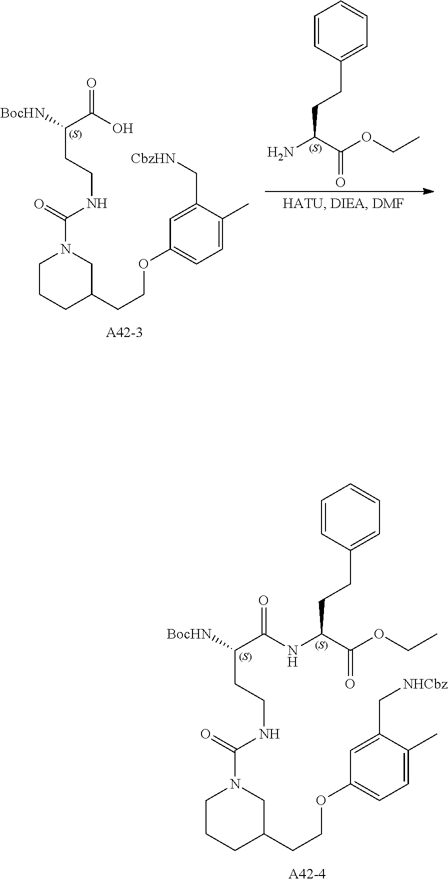

To a solution of ethyl (2S)-2-amino-4-phenyl-butanoate (0.13 g, 0.533 mmol, 1 eq, HCl), A42-3 (351 mg, 0.560 mmol, 1.05 eq), DIEA (276 mg, 2.13 mmol, 4 eq) in DMF (1 mL) and DCM (4 mL) was added HATU (223 mg, 0.587 mmol, 1.1 eq) at 0° C. The reaction was stirred at 0-10° C. for 1 h to give a yellow mixture. LCMS showed the reaction was completed. H₂O (10 mL) was added. The mixture was concentrated under reduced pressure to give a residue. The residue was partitioned between EA (60 mL) and H₂O (20 mL). The organic layer was washed with H₂O (20 mL×2), brine (20 mL), dried over anhydrous Na₂SO₄, filtered and concentrated under reduced pressure to give yellow oil. The crude product was purified by combi flash (EA/PE=0/1 to 1/1) to give A42-4 (0.5 g, 100% yield, 87% purity) as yellow oil.

### 4. Preparation of A42-5:

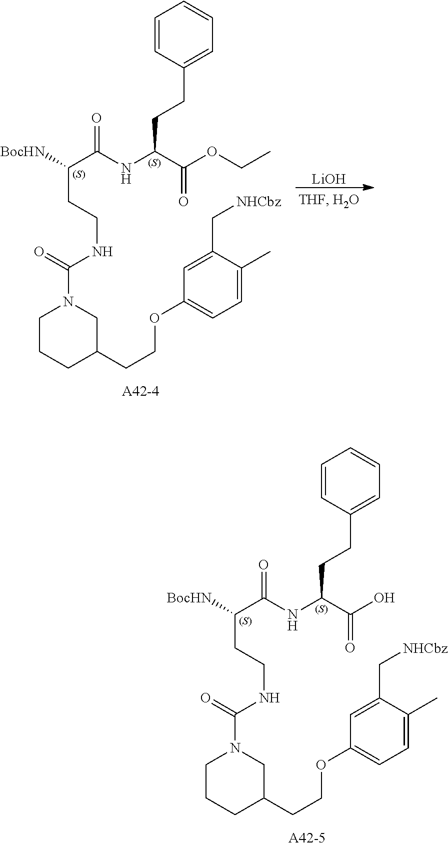

To a solution of A42-4 (0.5 g, 0.613 mmol, 1 eq) in THF (5 mL) was added LiOH·H₂O (30 mg, 0.705 mmol, 1.15 eq) in H₂O (1 mL). The reaction was stirred at 20° C. for 1 h to give a yellow mixture. LCMS showed the starting material was not consumed completely. A solution of LiOH H₂O (20 mg) in H₂O (1 mL) was added. The reaction was stirred at 20° C. for 0.5 h to give a yellow mixture. LCMS showed the reaction was completed. H₂O (15 mL) was added. Then the aqueous layer was acidified to pH=4 by addition of 1 M HCl. The aqueous layer was extracted with DCM (20 mL×2). The combined organic layer was washed with brine (10 mL), dried over anhydrous Na₂SO₄, filtered and concentrated in vacuum to give A42-5 (0.42 g, 87% yield) as yellow oil.

### 5. Preparation of A42-6:

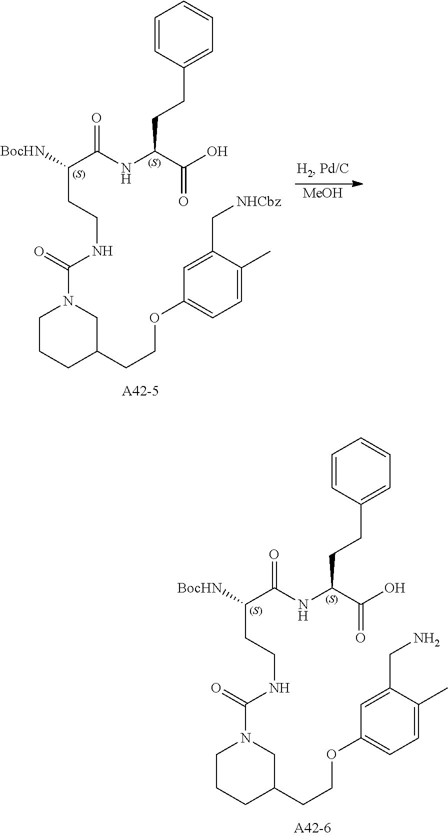

To a solution of A42-5 (0.42 g, 0.533 mmol, 1 eq) in EtOH (6 mL) and DCM (6 mL) was added Pd/C (0.15 g, 10% purity). The reaction suspension was stirred at 20° C. under 15 psi of H₂ for 17 h to give a black suspension. LCMS showed the starting material was consumed completely. The reaction mixture was filtered through a pad of celite cake. The filtrate was concentrated in vacuum to give A42-6 (0.28 g, 80% yield) as white gum.

### 6. Preparation of A42-7:

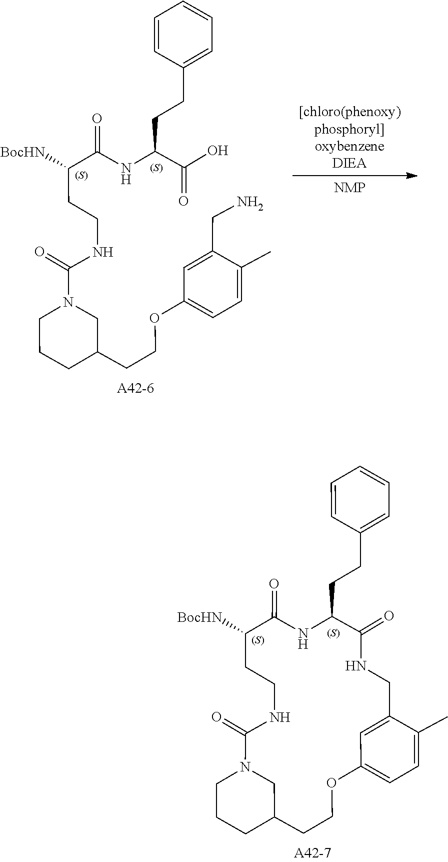

To a solution of ethyl (2E)-2-cyano-2-hydroxyimino-acetate (122 mg, 0.856 mmol, 2 eq), 4-methylmorpholine (260 mg, 2.57 mmol, 6 eq), [chloro(phenoxy)phosphoryl]oxybenzene (230 mg, 0.857 mmol, 2 eq) in NMP (2 mL) and DCM (5 mL) was added A42-6 (0.28 g, 0.428 mmol, 1 eq) and the reaction was stirred at 20° C. for 1.5 h to give a yellow solution. LCMS showed the starting material was consumed completely. H₂O (15 mL) was added. The resultant solution was concentrated to remove DCM. The residue was partitioned between EA (80 mL) and H₂O (80 mL). The organic layer was washed with water (80 mL*2), brine (80 mL), dried over anhydrous Na₂SO₄, concentrated under reduced pressure to give a yellow oil. The mother liquid was purified by combi flash (EA/PE=0/1 to 3/1) to give A42-7 (0.15 g, 44% yield, 80% purity) as white solid.

### 7. Preparation of 8:

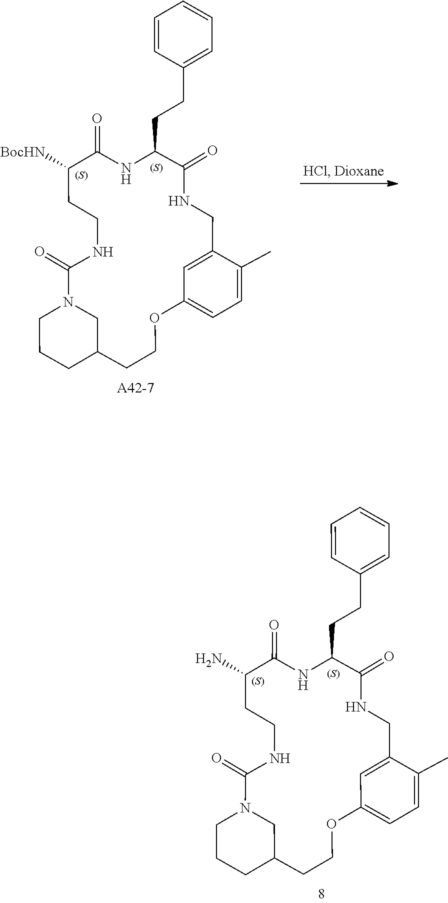

To a solution of A42-7 (150 mg, 0.236 mmol, 1 eq) in dioxane (1 mL) was added HCl/dioxane (4 M, 1.18 mL, 20 eq). The reaction was stirred at 20° C. for 30 min to give a yellow solution. LCMS showed the starting material was consumed completely. HCl/dioxane (4 M, 0.5 mL) was added. The reaction was stirred at 20° C. for 30 min to give a yellow solution. LCMS showed the starting material was consumed completely. The solution was concentrated under reduced pressure to give 8 (141 mg, crude, HCl) as a white solid.

### 8. Preparation of 653:

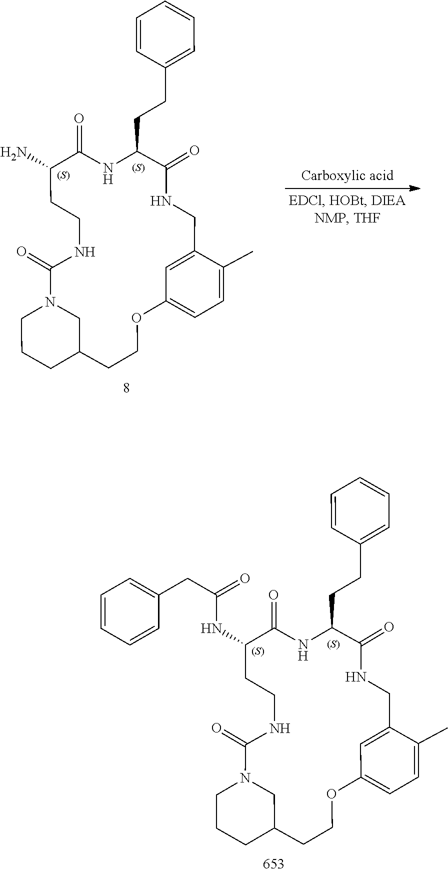

A solution of 8 (0.13 g, 0.227 mmol, 1 eq, HCl), 2-phenylacetic acid (40 mg, 0.295 mmol, 1.3 eq), 1-hydroxybenzotriazole (43 mg, 0.318 mmol, 1.4 eq), DIEA (88 mg, 0.682 mmol, 3 eq) in DCM (3 mL) and THF (3 mL) was stirred at 20° C. for 15 min to give a yellow mixture. 3-(ethyliminomethyleneamino)-N,N-dimethyl-propan-1-amine;hydrochloride (61 mg, 0.318 mmol, 1.4 eq) were added. The reaction was stirred at 20° C. for 1 h to give a yellow mixture. LCMS showed the starting material was consumed completely. The solution was partitioned between EA (100 mL) and H₂O (20 mL). The organic layer was washed with 0.2 M HCl (40 mL), sat. NaHCO₃ (30 mL, brine (30 mL), dried over anhydrous Na₂SO₄, concentrated under reduced pressure to give yellow oil. The yellow oil was purified by prep-HPLC(column: 2_Phenomenex Gemini C18 75*40 mm*3 um; mobile phase: [water(NH₃H₂O+NH₄HCO₃) -ACN];B %: 50%-80%, 7.8 min). The eluent was lyophilized to give 653 (99.4 mg, 67% yield, 100% purity) as a white powder.

¹H NMR (400 MHz, DMSO-d₆) δ ppm 1.12-2.04 (m, 11H), 2.15 (s, 3H), 2.60-3.24 (m, 5H), 3.52 (s, 2H), 3.57-4.39 (m, 9H), 6.30-7.04 (m, 3H), 7.00 (d, J=8 Hz, 1H), 7.09-7.35 (m, 10H), 8.06-8.50 (m, 3H)

LCMS: 100%, MS (ESI): m/z 654.3 [M+H]⁺.

### Example A-45: Preparation of Macrocyclic Compound 593

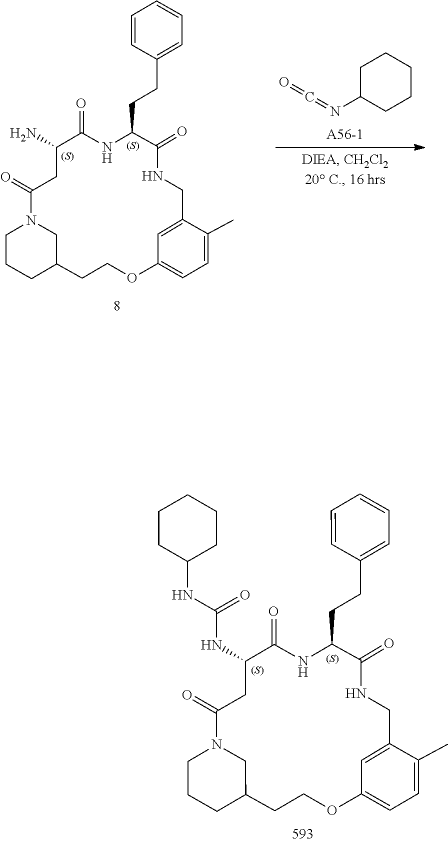

To a solution of 8 (100 mg, 184.13 umol, 1 eq, HCl) in DCM (5 mL) was added DIEA (71.39 mg, 552.39 umol, 96.22 uL, 3 eq) and A56-1(34.57 mg, 276.19 umol, 35.28 uL, 1.5 eq). The mixture was stirred at 20° C. for 16 hrs to give a yellow solution. The reaction mixture was filtered. The filter cake was washed with DCM (10 mL) dried in concentrated. The crude product was by trituration with DCM:EtOAc (5:1, 6 ml) to afford 593 (66.13 mg, 104.67 umol, 56.85% yield, 100% purity) as white solid.

¹H NMR (400 MHz, DMSO-d₆) δ=8.34-7.88 (m, 2H), 7.30-7.13 (m, 5H), 7.03 (dd, J=3.8, 8.3 Hz, 1H), 6.97-6.64 (m, 2H), 6.12-5.97 (m, 2H), 4.64-3.70 (m, 8H), 3.03-2.59 (m, 5H), 2.34-2.11 (m, 4H), 2.00-0.97 (m, 20H)

LCMS: 100%, MS (ESI): m/z 632.3 [M+H]⁺.

### Example A-46: Preparation of Macrocyclic Compound 624

### 1. Preparation of A44-1:

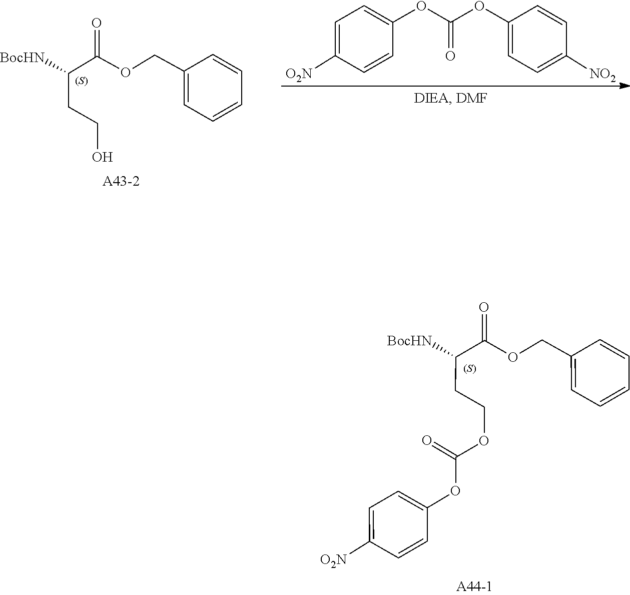

To a solution of A43-2 (508 mg, 1.64 mmol, 1 eq), bis(4-nitrophenyl) carbonate (0.5 g, 1.64 mmol, 1 eq) in DMF (8 mL) was added DIEA (637 mg, 4.93 mmol, 3 eq). The reaction was stirred at 20° C. for 17 h to give a yellow mixture. LCMS showed desired MS value was detected. The mixture was partitioned between EA (60 mL) and H₂O (40 mL). The organic layer was washed with H₂O (40 mL), brine (40 mL), dried over anhydrous Na₂SO₄, filtered and concentrated under reduced pressure to give yellow oil. The yellow oil was purified by combi flash (EA/PE=0/1 to 1/9) to give A44-1(0.72 g, 89% yield, 97% purity) as yellow oil.

### 2. Preparation of A44-2:

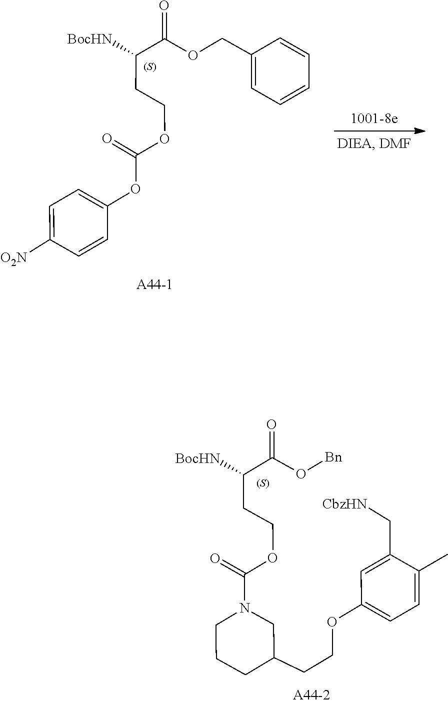

To a solution of A44-1(0.6 g, 1.26 mmol, 1 eq), 1001-8e (657 mg, 1.39 mmol, 1.1 eq, oxalic acid) in DMF (12 mL) was added DIEA (490 mg, 3.79 mmol, 3 eq). The reaction was stirred at 20° C. for 17 h to give a yellow mixture. LCMS showed desired MS value was detected. The mixture was partitioned between EA (100 mL) and H₂O (60 mL). The organic layer was washed with H₂O (60 mL), brine (60 mL), dried over anhydrous Na₂SO₄, filtered and concentrated under reduced pressure to give yellow oil. The yellow oil was purified by combi flash (EA/PE=0/1 to 1/3) to give A44-2 (0.88 g, 94% yield, 97% purity) as colorless oil.

### 3. Preparation of A44-3:

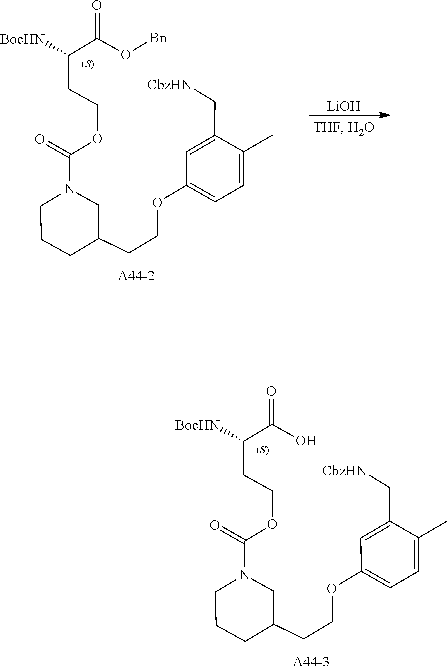

To a solution of A44-2 (0.88 g, 1.23 mmol, 1 eq) in THF (10 mL) was added LiOH·H₂O (59 mg, 1.41 mmol, 1.15 eq) in H₂O (2 mL). The reaction was stirred at 20° C. for 2.5 h to give a yellow mixture. LCMS showed the reaction was completed. H₂O (15 mL) was added. Then the aqueous layer was acidified to pH=4 by addition of 1 M HCl. The aqueous layer was extracted with EA (40 mL×2). The combined organic layer was washed with brine (30 mL), dried over anhydrous Na₂SO₄, filtered and concentrated in vacuum to give A44-3 (0.8 g, crude) as yellow oil.

### 4. Preparation of A44-4:

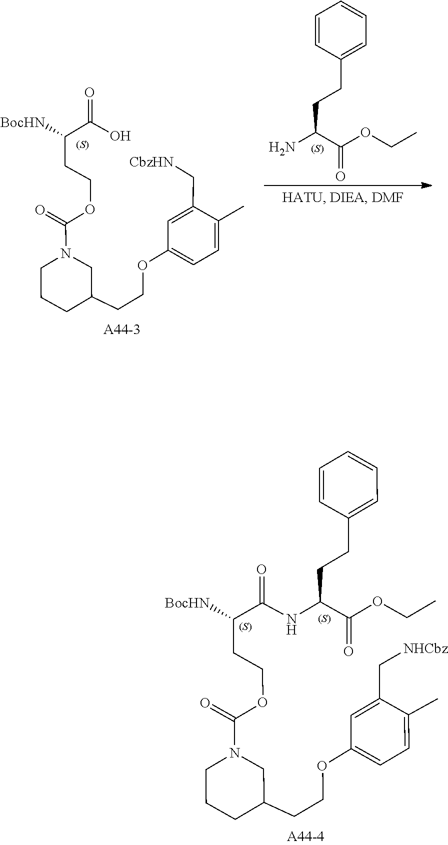

To a solution of ethyl (2S)-2-amino-4-phenyl-butanoate (0.38 g, 1.56 mmol, 1 eq, HCl), A44-3 (1.03 g, 1.64 mmol, 1.05 eq), DIEA (806 mg, 6.24 mmol, 4 eq) in DMF (4 mL) and DCM (16 mL) was added HATU (652 mg, 1.72 mmol, 1.1 eq) at 0° C. The reaction was stirred at 0-10° C. for 1 h to give a yellow mixture. LCMS showed the reaction was completed. H₂O (10 mL) was added. The mixture was concentrated under reduced pressure to give a residue. The residue was partitioned between EA (60 mL) and H₂O (20 mL). The organic layer was washed with H₂O (20 mL×2), brine (20 mL), dried over anhydrous Na₂SO₄, filtered and concentrated under reduced pressure to give yellow oil. The crude product was purified by combi flash (EA/PE=0/1 to 2/5) to give A44-4 (1.16 g, 91% yield) as colorless oil.

### 5. Preparation of A44-5:

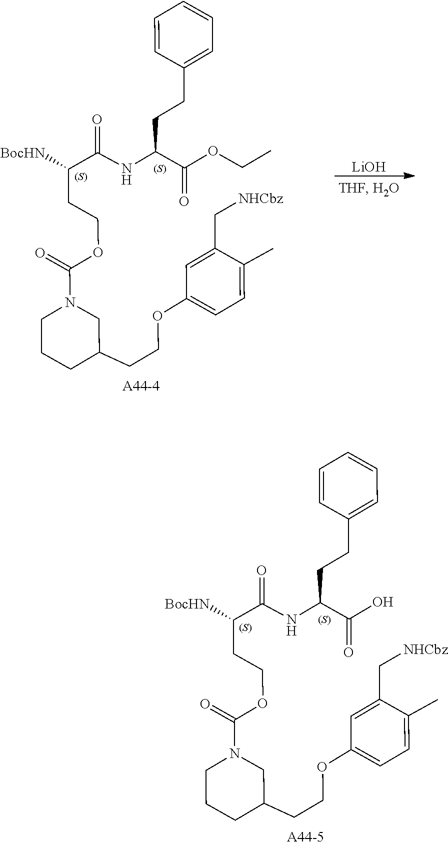

To a solution of A44-4 (1.16 g, 1.42 mmol, 1 eq) in THF (12 mL) was added LiOH·H₂O (68 mg, 1.63 mmol, 1.15 eq) in H₂O (3 mL). The reaction was stirred at 20° C. for 3 h to give a yellow mixture. LCMS showed the starting material was not consumed completely. A solution of LiOH H₂O (30 mg) in H₂O (1 mL) was added. The reaction was stirred at 20° C. for 1 h to give a yellow mixture. LCMS showed the reaction was completed. H₂O (15 mL) was added. Then the aqueous layer was acidified to pH=4 by addition of 1 M HCl. The aqueous layer was extracted with EA (30 mL×2). The combined organic layer was washed with brine (20 mL), dried over anhydrous Na₂SO₄, filtered and concentrated in vacuum to give A44-5 (1.14 g, crude) as yellow oil.

### 6. Preparation of A42-6:

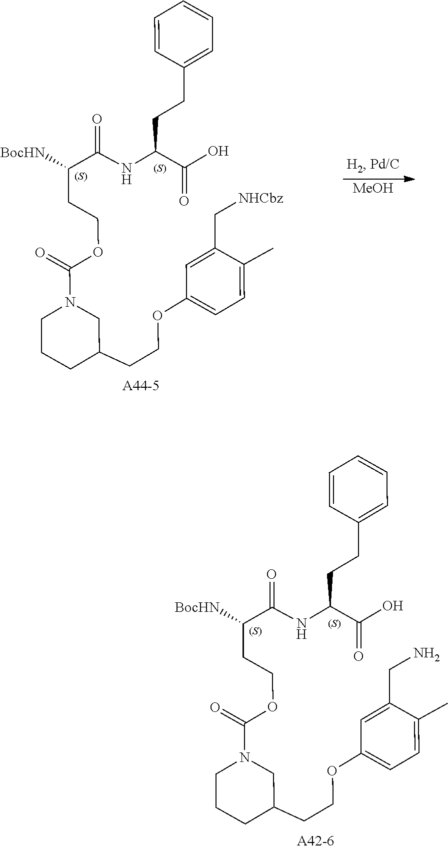

To a solution of A44-5 (1.12 g, 1.42 mmol, 1 eq) in EtOH (20 mL) and DCM (20 mL) was added Pd/C (0.4 g, 10% purity). The reaction suspension was stirred at 20° C. under 15 psi of H₂ for 17 h to give a black suspension. LCMS showed the starting material was consumed completely. The reaction mixture was filtered through a pad of celite cake. The filtrate was concentrated in vacuum to give A42-6 (0.91 g, 97% yield) as white gum.

### 7. Preparation of A42-7:

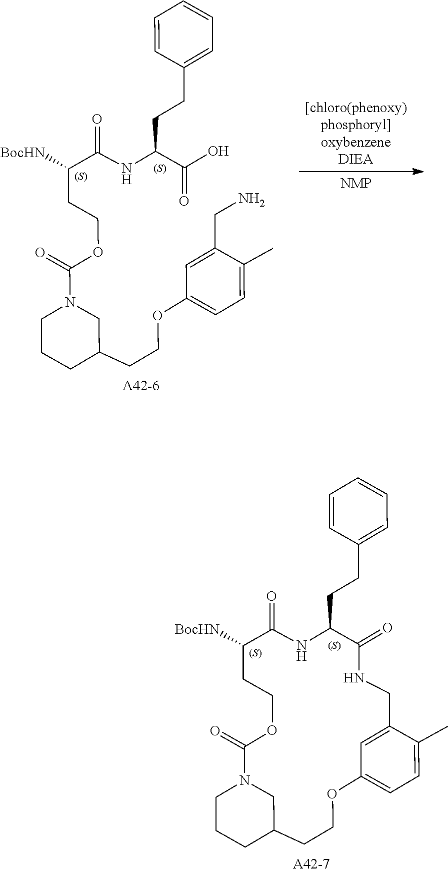

To a solution of ethyl (2E)-2-cyano-2-hydroxyimino-acetate (395 mg, 2.78 mmol, 2 eq), 4-methylmorpholine (843 mg, 8.34 mmol, 6 eq), [chloro(phenoxy)phosphoryl]oxybenzene (747 mg, 2.78 mmol, 2 eq) in NMP (4 mL) and DCM (12 mL) was added A42-6 (910 mg, 1.39 mmol, 1 eq) and the reaction was stirred at 20° C. for 1.5 h to give a yellow solution. LCMS showed the starting material was consumed completely. H₂O (15 mL) was added. The resultant solution was concentrated to remove DCM. The residue was partitioned between EA (80 mL) and H₂O (80 mL). The organic layer was washed with water (80 mL*2), brine (80 mL), dried over anhydrous Na₂SO₄, concentrated under reduced pressure to give a yellow oil. The mother liquid was purified by combi flash (EA/PE=0/1 to 2/3) to give A42-7 (230 mg, 23% yield, 89% purity) as white solid.

### 8. Preparation of Compound 8:

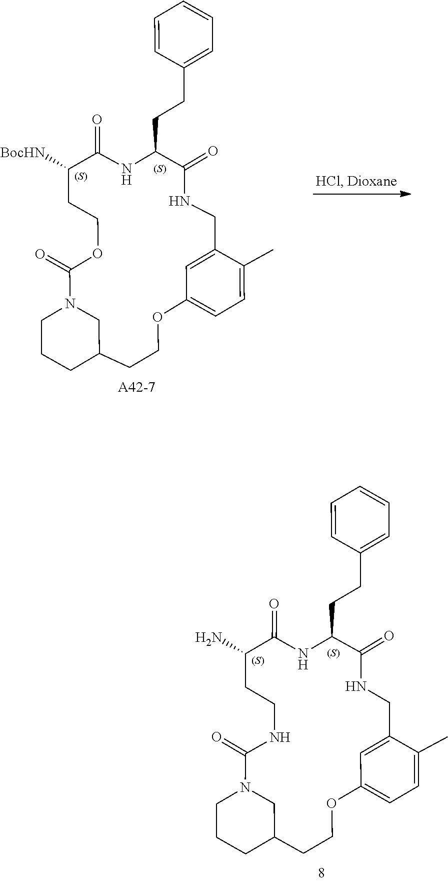

To a solution of A42-7 (230 mg, 0.361 mmol, 1 eq) in dioxane (1 mL) was added HCl/dioxane (4 M, 1.81 mL, 20 eq). The reaction was stirred at 20° C. for 1 h to give a yellow solution. LCMS showed the starting material was consumed completely. The solution was concentrated under reduced pressure to give 8 (0.2 g, 96% yield, HCl) as a white solid.

### 9. Preparation of Compound 624:

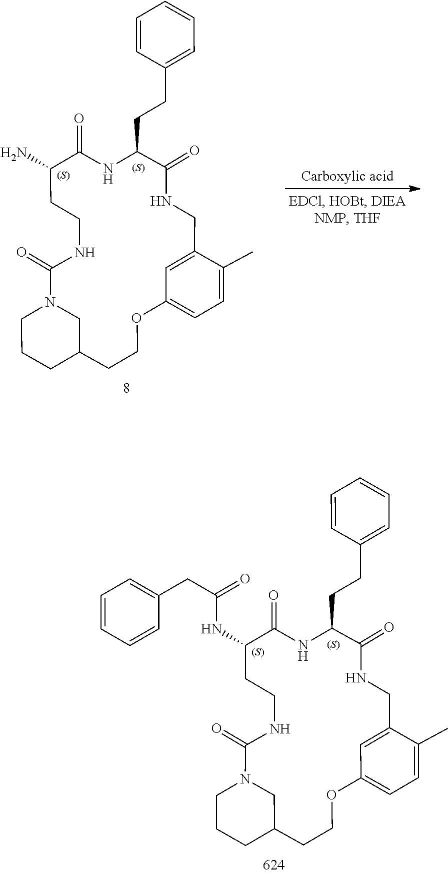

A solution of 8 (100 mg, 0.174 mmol, 1 eq, HCl), 2-phenylacetic acid (31 mg, 0.227 mmol, 1.3 eq), 1-hydroxybenzotriazole (33 mg, 0.244 mmol, 1.4 eq), DIEA (68 mg, 0.523 mmol, 3 eq) in DCM (3 mL) and THF (3 mL) was stirred at 20° C. for 15 min to give a yellow mixture. 3-(ethyliminomethyleneamino)-N,N-dimethyl-propan-1-amine;hydrochloride (47 mg, 0.244 mmol, 1.4 eq) were added. The reaction was stirred at 20° C. for 1 h to give a yellow mixture. LCMS showed the starting material was consumed completely. The solution was partitioned between EA (60 mL) and H₂O (20 mL). The organic layer was washed with 0.2 M HCl (40 mL), sat. NaHCO₃ (30 mL), brine (30 mL), dried over anhydrous Na₂SO₄, concentrated under reduced pressure to give yellow oil. The yellow oil was purified by prep-HPLC (column: 2_Phenomenex Gemini C18 75*40 mm*3 um; mobile phase: [water(NH₃H₂O+NH₄HCO₃)-ACN];B %: 52%-82%, 7.8 min). The eluent was lyophilized to give 624 (65.9 mg, 57% yield, 100% purity) as a white powder.

¹H NMR (400 MHz, DMSO-d₆) δ ppm 1.12-2.09 (m, 11H), 2.16 (s, 3H), 2.56-3.05 (m, 4H), 3.49-4.44 (m, 12H), 6.58-7.09 (m, 3H), 7.09-7.39 (m, 10H), 7.89-8.58 (m, 3H)

LCMS: 100%, MS (ESI): m/z 655.3 [M+H]⁺.

### Example A-47: Preparation of Macrocyclic Compound 740

### 1. Preparation of A149-2a:

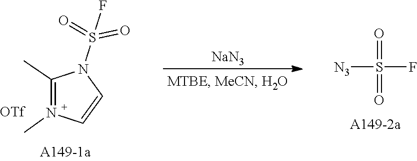

To a mixture of NaN₃ (103.97 mg, 1.60 mmol, 1.05 eq) in H₂O (4 mL) and MTBE (4 mL) was added A149-1a (500 mg, 1.52 mmol, 1 eq) in MeCN (0.5 mL) at 0° C., and then the resulting mixture was stirred at 0-5° C. for 30 min to give a colorless mixture. The mixture was partitioned between MTBE (4 mL) and water (4 mL). The organic layer was monitored by ¹⁹F-NMR. Obtained A149-2a (180 mg, crude) in MTBE (4 mL), which was used directly.

### 2. Preparation of A149-1:

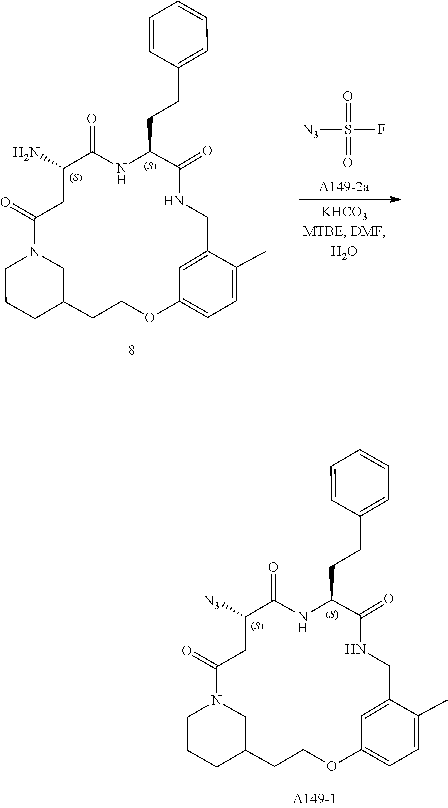

To a mixture of 8 (150 mg, 0.296 mmol, 1 eq) and A149-2a in MTBE (4 mL) and DMF (1 mL) was added KHCO₃ (59.28 mg, 0.592 mmol, 2 eq) in H₂O (1 mL). The resulting mixture was stirred at 20-25° C. for 20 h to give a colorless mixture. TLC showed the reaction was completed. The mixture was diluted with H₂O (20 mL), extracted with EA (20 mL×2), washed with water (20 mL×3). The combined organic layer was concentrated to dryness. The residue was purified by flash column (THF/PE=40-60%, SiO₂) to afford A149-1(50 mg, 0.093 mmol, 31.71% yield) as a white solid.

### 3. Preparation of Compound 740:

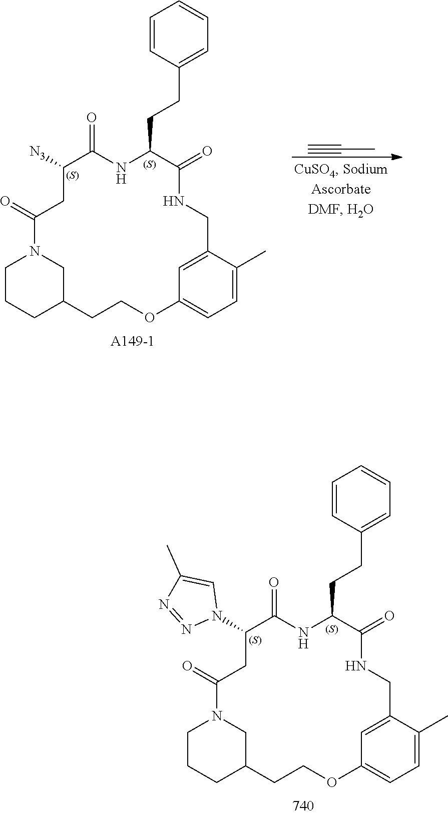

A mixture of A149-1(50 mg, 0.65 mmol, 1 eq), prop-1-yne (1 M, 0.394 mL, 6 eq), CuSO₄ (2.10 mg, 0.013 mmol, 0.002 mL, 0.2 eq) and Sodium Ascorbate (6.51 mg, 0.032 mmol, 0.5 eq) in DMF (0.5 mL) and H₂O (0.3 mL) was stirred at 45° C. for 2 h under N₂ to give a yellow mixture. TLC showed new spot. The mixture was added 10 mL EA, filtered by a pad of celite. Then the mixture was diluted with H₂O (10 mL), extracted with EA (10 mL×2), washed with water (10 mL×2). The combined organic layer was concentrated to dryness. The residue was purified by flash column (EA in PE=40-100%, SiO₂) to afford 740 (15.1 mg, 0.026 mmol, 40.12% yield) as a white powder.

¹H NMR (400 MHz, DMSO-d₆): δ=8.93-8.94 (m, 1H), 7.89-8.45 (m, 2H), 7.19-7.30 (m, 3H), 6.98-7.13 (m, 1H), 6.82-6.93 (m, 1H), 6.65-6.80 (m, 1H), 5.50-5.80 (m, 1H), 4.17-4.26 (m, 6H), 3.50-3.90 (m, 2H), 2.60-3.30 (m, 4H), 1.76-2.20 (m, 12H), 1.23-1.75 (m, 6H).

HPLC: 96.67%, MS (ESI): m/z 573.3 [M+H]⁺.

### Example A-48: Preparation of Macrocyclic Compound 632

### 1. Preparation of A57-2a:

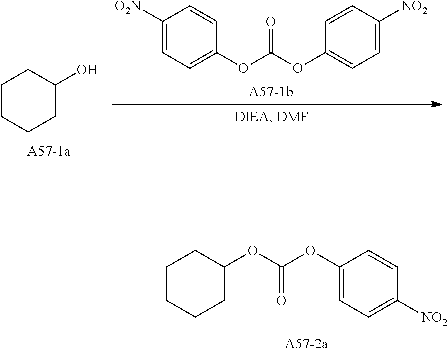 [SMILES: O=C(Oc1ccc([N+](=O)[O-])cc1)Oc1ccc([N+](=O)[O-])cc1 ; OC1CCCCC1 ; O=C(Oc1ccc([N+](=O)[O-])cc1)OC1CCCCC1]

To a solution of A57-1a (127 mg, 1.27 mmol, 132.29 uL, 1 eq) and A57-1b (1.16 g, 3.80 mmol, 3 eq.) in DMF (3 mL) was added DIEA (491.64 mg, 3.80 mmol, 662.58 uL, 3 eq). The mixture was stirred at 25° C. for 3 h. The solution was partitioned between EA (20 mL) and H₂O (10 mL). The organic layer was washed with H₂O (10 mL×3), brine (10 mL), dried over anhydrous Na₂SO₄, filtered and concentrated under reduced pressure. The crude was purified by column chromatography (SiO₂, PE/EA=80/20) to afford (306 mg, 1.15 mmol, 90.98% yield) as yellow solid.

### 2. Preparation of Compound 632:

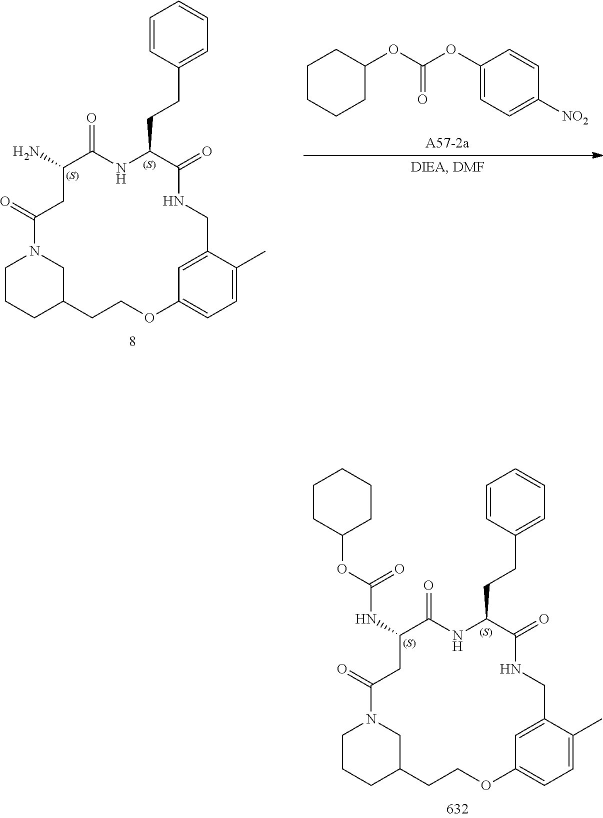

To a solution of A57-2a (306 mg, 1.15 mmol, 1 eq) and 8 (584.44 mg, 1.15 mmol, 1 eq) in DMF (5 mL) was added DIEA (447.28 mg, 3.46 mmol, 602.80 uL, 3 eq). The mixture was stirred at 25° C. for 3h as colorless solution. The solution was partitioned between EA (20 mL) and H₂O (10 mL). The organic layer was washed with H₂O (10 mL×3), brine (1 mL), dried over anhydrous Na₂SO₄, filtered and concentrated under reduced pressure. The crude product was purified by pre-HPLC (column: ACE 5 C18-AR 150*30 mm*5 μm; mobile phase: [water (HCl)-ACN]; B %: 55%-85%, 8 min) to afford 632 (318 mg, 502.54 umol, 43.56% yield) as white solid. 1H NMR (400 MHz, DMSO-d₆) δ=8.46-7.77 (m, 2H), 7.52-7.10 (m, 6H), 7.03-6.58 (m, 3H), 4.57-3.79 (m, 9H), 2.80-2.53 (m, 5H), 2.20-2.13 (m, 3H), 1.99-1.11 (m, 20H)

LCMS: 96.1%, MS (ESI): m/z 633.7 [M+H]⁺

### Example A-49: Preparation of Macrocyclic Compound 628

### 1. Preparation of A69-2a:

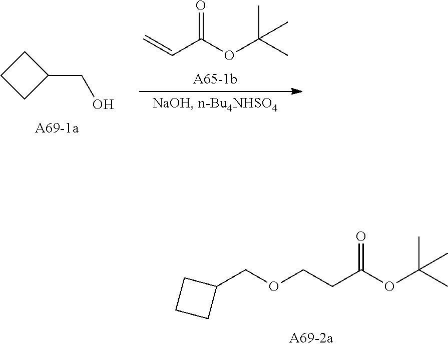

To a solution of A65-1b (1.34 g, 10.45 mmol, 1.52 mL, 3 eq), NaOH (5 M, 696.61 uL, 1 eq) n-BuNHSO₄ (295.65 mg, 870.76 umol, 0.25 eq) in DCM (3 mL) was added A69-1a (300 mg, 3.48 mmol, 0.328 mL, 1 eq) at 0° C., the resulting mixture was stirred at 20° C. for 14 hours to give white solution. The reaction mixture was poured into water (5 mL) and extracted with EtOAc (5 mL×4). The combined organic layers were washed with brine (10 mL), dried over Na₂SO₄, filtered and concentrated under reduced pressure. The crude product was purified by flash column (SiO₂, PE to 10% EtOAc in PE) to afford A69-2a (500 mg, 2.33 mmol, 66.99% yield) was obtained as white oil.

### 2. Preparation of A69-3a:

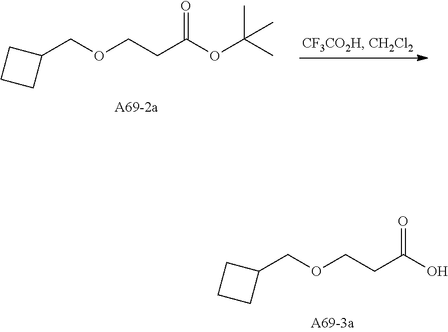

To a solution of A69-2a (500 mg, 2.33 mmol, 1 eq) in DCM (3 mL) was added CF₃COOH CF₃COOH (1.54 g, 13.51 mmol, 1 mL, 5.79 eq) at 0° C., the resulting mixture was stirred at 20° C. for 4 hours to give white solution. The reaction mixture was concentrated directly to afford A69-3a (360 mg, crude) was obtained as yellow oil.

### 3. Preparation of Compound 628:

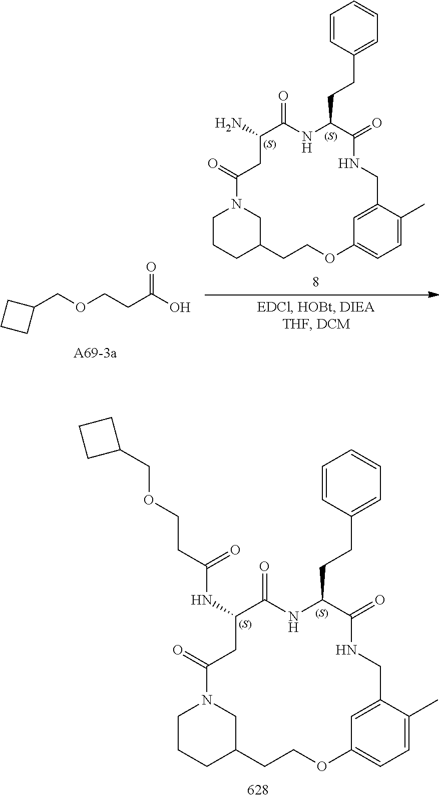

To a solution of 8 (130 mg, 0.239 mmol, 1 eq, HCl), A69-3a (49.23 mg, 0.311 mmol, 1.3 eq), HOBt (45.28 mg, 0.335 mmol, 1.4 eq), DIEA (92.81 mg, 0.718 mmol, 0.125 mL, 3 eq) in THF (2 mL) and DCM (2 mL) was stirred at 20° C. for 15 min to give a yellow mixture. Then EDCl (64.24 mg, 0.335 mmol, 1.4 eq) were added. The reaction was stirred at 20° C. for 1 h to give a yellow mixture. The reaction mixture was poured into water (10 mL) and extracted with EtOAc (10 mL×4). The organic layer was washed with 0.2 M HCl (20 mL), sat.NaHCO₃ (30 mL), brine (30 mL), dried over anhydrous Na₂SO₄, concentrated under reduced pressure to give yellow oil. The residue was purified by prep-HPLC (basic, column: 2_Phenomenex Gemini C18 75*40 mm*3 um; mobile phase: [water (NH₃H₂O+NH₄HCO₃)-ACN]; B %: 54%-84%, 7.8 min). The afforded flows were combined, concentrated to remove most of CH₃CN and lyophilized to afford 628 (53.09 mg, 0.082 mmol, 34.29% yield, 100% purity) was obtained as white solid.

¹H NMR (400 MHz, DMSO-d₆) δ=8.40-7.77 (m, 3H), 7.29-6.63 (m, 9H), 4.75-3.49 (m, 11H), 3.04-2.54 (m, 6H), 2.41-2.28 (m, 3H), 2.20-2.11 (m, 4H), 2.00-1.15 (m, 16H)

LCMS: 100%, Time t1=4.151 min, MS (ESI): m/z 647.3 [M+H]⁺.

### Example A-50: Preparation of Macrocyclic Compound 739

### 1. Preparation of A61-2a:

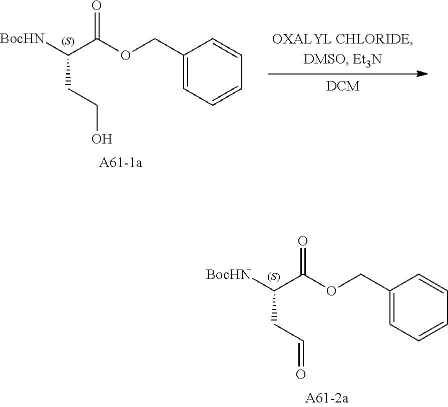

To a solution of oxalyl chloride (923.15 mg, 7.27 mmol, 636.66 uL, 1.5 eq) in DCM (15 mL) at −78° C. was added DMSO (947.13 mg, 12.12 mmol, 947.13 uL, 2.5 eq) in DCM (5 mL). After 20 min. A61-1a (1.5 g, 4.85 mmol, 1 eq) in DCM (5 mL) was added and the reaction stirred for 45 min at −78° C. Then Et₃N (2.45 g, 24.24 mmol, 3.37 mL, 5 eq) was added and the mixture was stirred at 25° C. for 1 h. LCMS showed a desired mass. The reaction mixture was diluted with NaHCO₃ (60 mL), and extracted with EA (70 mL×2). The organic layer was washed with H₂O (50 mL×2), brine (60 mL), dried over anhydrous Na₂SO₄, filtered and concentrated under reduced pressure to give A61-2a (1.39 g, crude) as yellow oil.

LCMS: 72.57%, MS (ESI): m/z 252.0 [M−56+H]⁺.

### 2. Preparation of A61-3a:

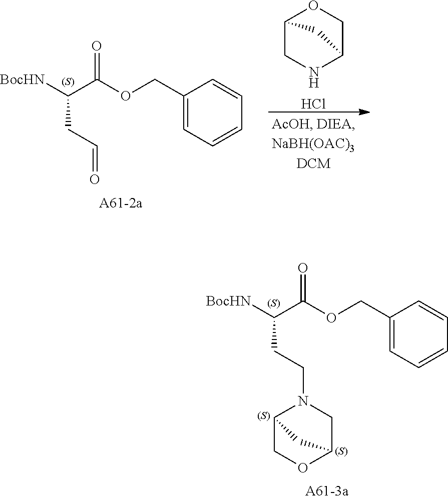

To a solution of A61-2a (500 mg, 1.63 mmol, 1 eq) and DIEA (420.52 mg, 3.25 mmol, 0.566 mL, 2 eq) in DCM (15 mL) was added (1S,4S)-2-oxa-5-azabicyclo[2.2.1]heptane;hydrochloride (441.18 mg, 3.25 mmol, 2 eq) and AcOH (195.39 mg, 3.25 mmol, 0.186 mL, 2 eq). The reaction stirred at 25° C. for 1.5 h. Then NaBH(OAc)₃ (689.60 mg, 3.25 mmol, 2 eq) was added and the mixture was stirred at 25° C. for 17 h. LCMS showed a desired mass. The reaction mixture was diluted with H₂O (10 mL) and extracted with EA (20 mL×2). The organic later was washed with H₂O (20 mL×2), brine (20 mL×2), dried over anhydrous Na₂SO₄, filtered and concentrated under reduced pressure to give A61-3a (690 mg, crude) as colorless oil.

LCMS: 80.19%, MS (ESI): m/z 391.1 [M+H]⁺.

### 3. Preparation of A61-4a:

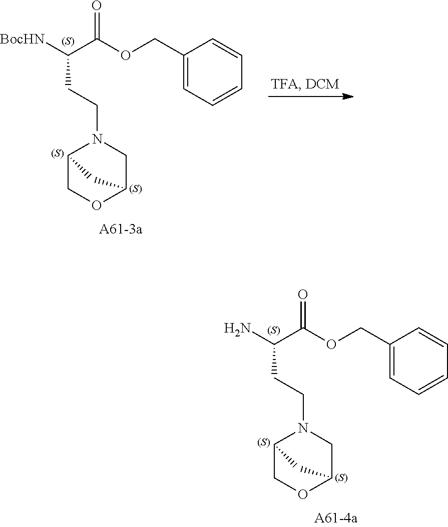

To a solution of A61-3a (640 mg, 1.64 mmol, 1 eq) in DCM (6 mL) was added TFA (2.46 g, 21.61 mmol, 1.6 mL, 13.18 eq). The reaction was stirred at 20° C. for 2 h to give a yellow solution. LCMS showed the reaction was completed. The solution was concentrated under reduced pressure to give A61-4a (830 mg, crude) as yellow oil.

LCMS: 73.53%, MS (ESI): m/z 291.1 [M+H]⁺.

### 4. Preparation of A61-1:

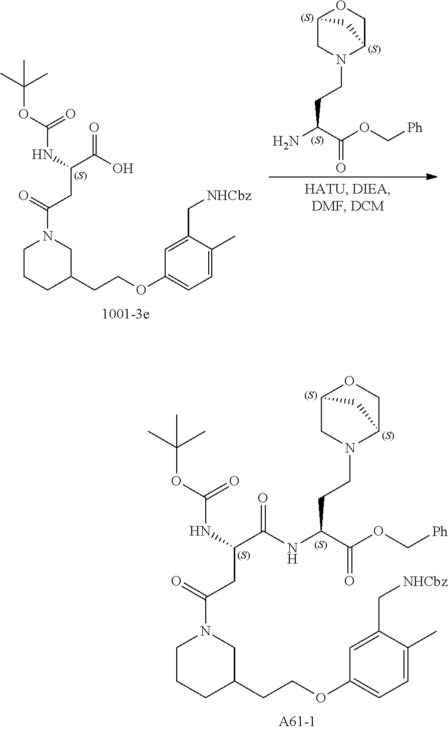

To a solution of 1001-3e (1 g, 1.67 mmol, 1 eq) and A61-4a (763.39 mg, 1.84 mmol, 70% purity, 1.1 eq) in DMF (6 mL) and DCM (8 mL) was added HATU (954.24 mg, 2.51 mmol, 1.5 eq) and DIEA (864.94 mg, 6.69 mmol, 1.17 mL, 4 eq). The mixture was stirred at 20° C. for 1 h to give a yellow solution. LCMS showed a desired mass. The reaction mixture was diluted with H₂O (60 mL) and extracted with EA (70 mL×2). The combined organic layers were washed with H₂O (60 mL×8) and with brine (60 mL×2), dried over Na₂SO₄, filtered and concentrated under reduced pressure to give a residue. The residue was purified by combi flash (SiO2, PE:EA=0% to 65%) to give A61-1 (670 mg, crude) as colorless oil.

LCMS: 71.88%, MS (ESI): m/z 870.3 [M+H]⁺.

### 5. Preparation of A61-2:

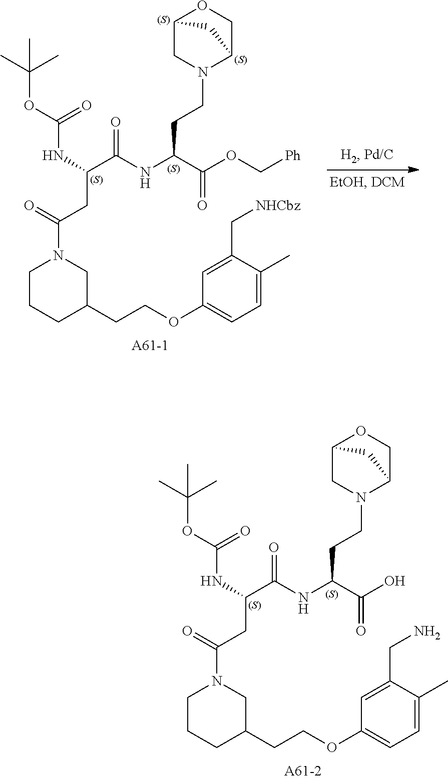

To a solution of A61-1(430 mg, 0.494 mmol, 1 eq) in EtOH (4 mL) and DCM (4 mL) was added Pd/C (0.4 g, 10% purity). The reaction suspension was stirred at 20° C. under 15 psi of H₂ for 16 h to give a black suspension. LCMS showed the starting material was consumed completely. The reaction mixture was filtered through a pad of celite cake. The filtrate was concentrated in vacuum to give A61-2 (320 mg, crude) as colorless oil.

LCMS: 69.13%, MS (ESI): m/z 646.4 [M+H]⁺.

### 6. Preparation of A61-3:

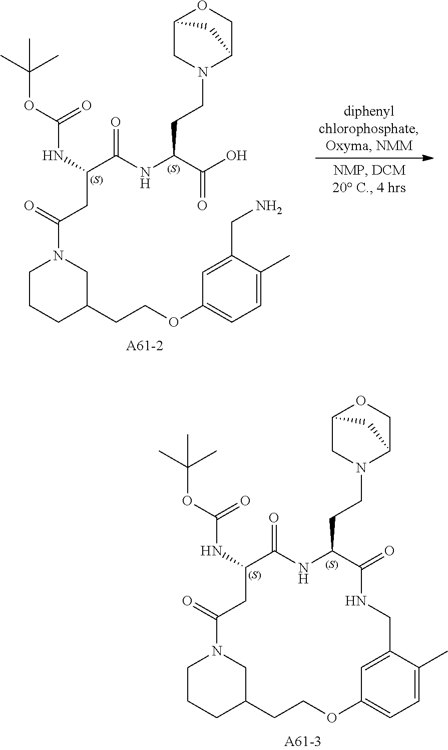

To a solution of ethyl (2E)-2-cyano-2-hydroxyimino-acetate (140.84 mg, 0.991 mmol, 2 eq), 4-methylmorpholine (300.73 mg, 2.97 mmol, 0.326 mL, 6 eq), [chloro(phenoxy)phosphoryl]oxybenzene (266.22 mg, 0.991 mmol, 0.204 mL, 2 eq) in NMP (2 mL) and DCM (5 mL) was added A61-2 (320 mg, 0.495 mmol, 1 eq) and the reaction was stirred at 20° C. for 1.5 h to give a yellow solution. LCMS showed the starting material was consumed completely. H₂O (20 mL) was added. The resultant solution was concentrated to remove DCM. The residue was partitioned between EA (80 mL) and H₂O (80 mL). The organic layer was washed with water (80 mL×2) and brine (80 mL), dried over anhydrous Na₂SO₄, concentrated under reduced pressure to give a yellow oil. The mother liquid was purified by combi flash (SiO2, MeOH/DCM=0:1 to 1:9) to give A61-3 (100 mg, crude) as yellow oil.

LCMS: 93.86%, MS (ESI): m/z 628.3 [M+H]⁺.

### 7. Preparation of A61-4:

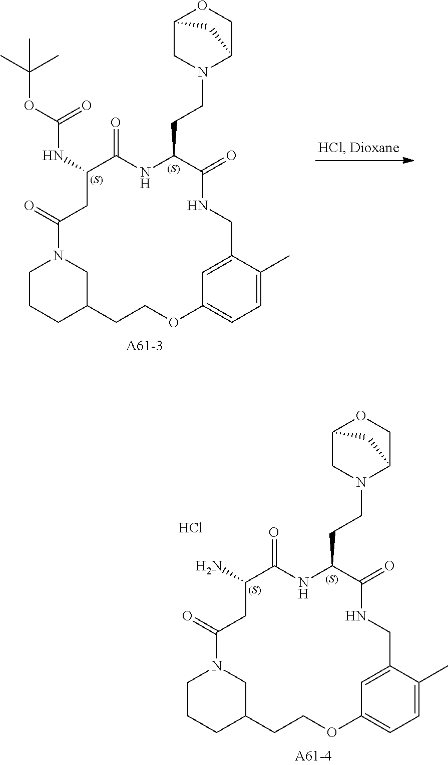

To a solution of A61-3 (100 mg, 0.159 mmol, 1 eq) in dioxane (2 mL) was added HCl/dioxane (4 M, 2 mL, 50.22 eq). The reaction was stirred at 20° C. for 2 h to give a yellow solution. LCMS showed the reaction was completed. The solution was concentrated under reduced pressure to give A61-4 (130 mg, crude, HCl) as a yellow solid.

LCMS: 84.18%, MS (ESI): m/z 528.2 [M+H]⁺.

### 8. Preparation of Compound 739:

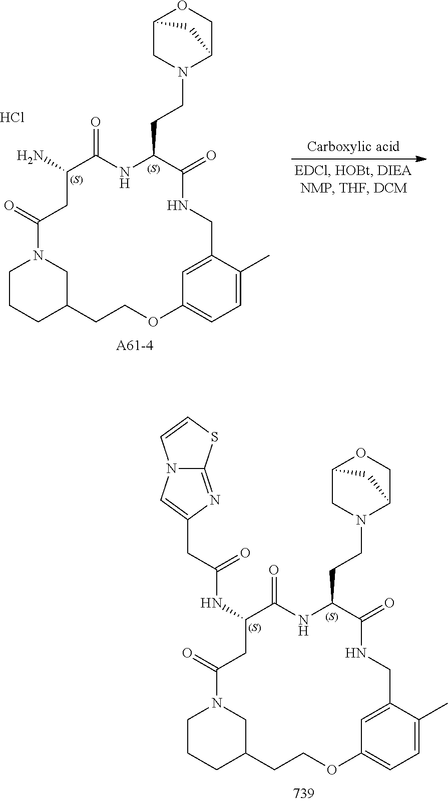

To a solution of A61-4 (130 mg, 0.230 mmol, 1 eq, HCl), 2-imidazo[2,1-b]thiazol-6-ylacetic acid (54.58 mg, 0.299 mmol, 1.3 eq), 1-hydroxybenzotriazole (43.59 mg, 0.322 mmol, 1.4 eq), DIEA (131.05 mg, 1.01 mmol, 0.176 mL, 4.4 eq) in DCM (3 mL) and THE (3 mL) was stirred at 20° C. for 15 min to give a yellow mixture. EDCl (61.85 mg, 0.322 mmol, 1.4 eq) were added. The reaction was stirred at 20° C. for 1.5 h to give a yellow mixture. LCMS showed the starting material was consumed completely. The solution was partitioned between EA (100 mL) and H₂O (20 mL). The organic layer was washed with 0.2 M HCl (20 mL), sat. NaHCO₃ (20 mL), brine (20 mL), dried over anhydrous Na₂SO₄, concentrated under reduced pressure to give yellow oil. The residue was purified by prep-HPLC (HCl).

Prep-HPLC was performed at conditions: Column: Phenomenex Gemini 21.2*100*5 um); Wavelength 220 nm; Mobile phase A: water (10 mM TFA); B acetonitrile; Flow rate: 25 mL/min; Injection volume: 2 mL; Run time: 10 min; Equilibration: 3 min.

The afforded flows were combined, concentrated to remove most of CH₃CN and lyophilized. Compound 739 (10 mg, 0.013 mmol, 6.03% yield, 96.21% purity) was obtained as yellow solid.

¹H NMR (400 MHz, DMSO-d₆) δ=8.81-7.85 (m, 5H), 7.70-7.55 (m, 1H), 7.12-6.60 (m, 3H), 4.71-3.86 (m, 14H), 3.14-2.64 (m, 7H), 2.38-1.90 (m, 7H), 1.86-1.08 (m, 8H)

LCMS: 96.21%, MS (ESI): m/z 692.6 [M+H]⁺.

### Example A-51: Preparation of Macrocyclic Compound 686

### 1. Preparation of A64-2:

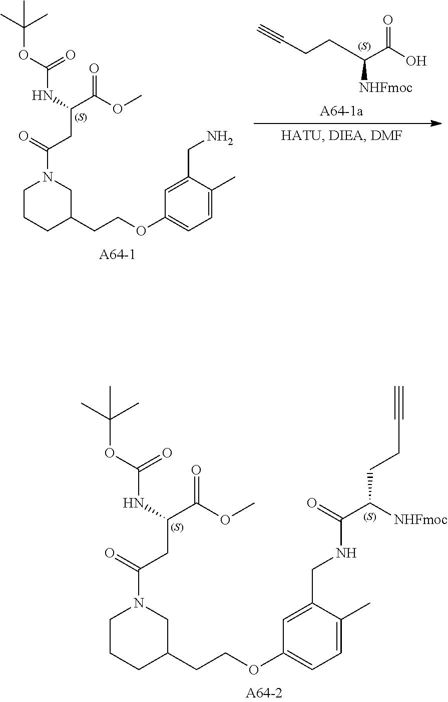

To a solution of A 64-1(300 mg, 628.15 umol, 1 eq) and A64-1a (219.46 mg, 628.15 umol, 1 eq) in DMF (6 mL) was added DIEA (324.74 mg, 2.51 mmol, 437.65 uL, 4 eq) and HATU (358.26 mg, 942.23 umol, 1.5 eq) at 0° C. The mixture was stirred at 25° C. for 2h to give a colorless solution. The mixture was diluted with EA (5 mL) and washed with H₂O (5 ML*3), brine (5 mL*2), then dried and concentrated under reduced pressure. The crude product was purified by flash column (SiO2, PE/EA=10/90) to afford A64-2 (400 mg, 494.46 umol, 78.72% yield) as yellow oil.

### 2. Preparation of A64-3:

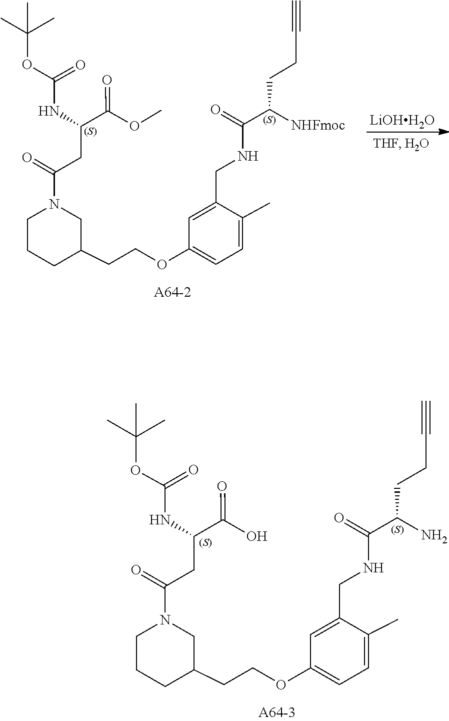

To a solution of A64-2 (400 mg, 494.46 umol, 1 eq) in THF (6 mL) and H₂O (1.5 mL) was added LiOH·H₂O (103.74 mg, 2.47 mmol, 5 eq). The mixture was stirred at 25° C. for 5h.

The mixture was acidized with HCl(1 M) to pH=6, and extracted with EA (300 mL*2). The organic layer was concentrated under reduced pressure. A64-3 (0.92 g, crude) was obtained as light yellow oil without purification.

### 3. Preparation of A64-4:

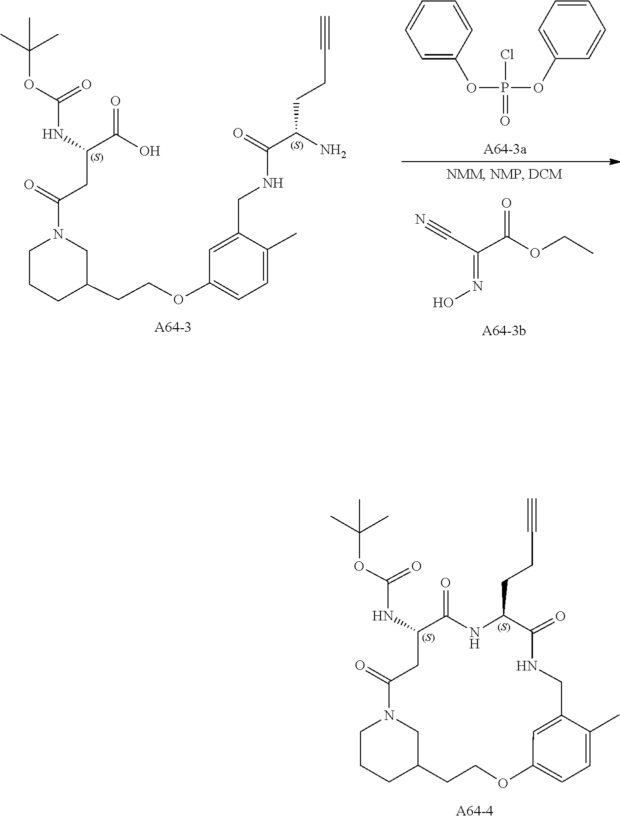

To a solution of A64-3a (70.97 mg, 499.40 umol, 2 eq), NMM (151.54 mg, 1.50 mmol, 164.71 uL, 6 eq), A64-3b (134.15 mg, 499.40 umol, 103.20 uL, 2 eq) in NMP (3 mL) and DCM (6 mL) was added A64-3 (143 mg, 249.70 umol, 1 eq). The mixture was stirred at 20° C. for 1.5 h to give a yellow solution. The mixture was partitioned between DCM (30 mL) and water (10 mL). The organic layer was washed with water (10 mL*5) and saturated brine (10 mL), dried over anhydrous Na₂SO₄, filtered, and concentrated under reduced pressure. The crude product was purified by pre-TLC (SiO2, PE/EA=1:1) to afford A64-4 (99 mg, crude) as yellow oil.

### 4. Preparation of A64-5:

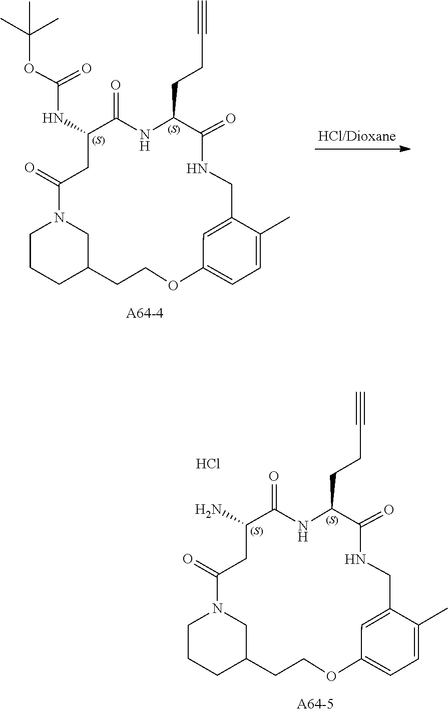

To a solution of A64-4 (99 mg, umol, 1eq.) in dioxane (20 mL) was added HCl/dioxane (4 M, 20 mL). The mixture was stirred at 20° C. for 2 hr under N₂ to give a colorless solution. The reaction was concentrated under reduced pressure. A64-5 (95 mg, 193.47 umol, 1 eq, HCl) was obtained as colorless oil.

### 5. Preparation of Compound 686:

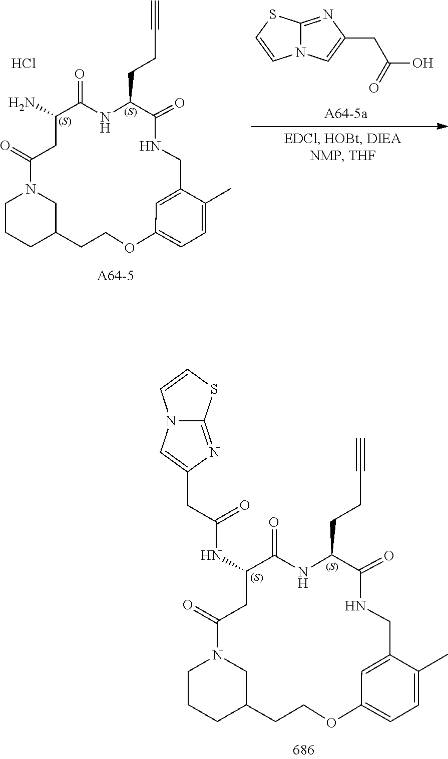

To a solution of A64-5 (95 mg, 193.47 umol, 1 eq, HCl), HOBt (36.60 mg, 270.86 umol, 1.4 eq), A64-5a (45.83 mg, 251.52 umol, 1.3 eq) and DIEA (75.02 mg, 580.42 umol, 101.10 uL, 3 eq) in DCM (2.5 mL) and THF (2.5 mL) was stirred 15 min and then EDCl (51.93 mg, 270.86 umol, 1.4 eq) was added. The mixture was stirred at 20° C. for 1 h. The mixture was partitioned between EA (100 mL) and H₂O (20 mL). The organic layer was washed with 0.2 MHCl (40 mL), sat. NaHCO₃ (30 mL), brine (30 mL), dried over anhydrous Na₂SO₄, concentrated under reduced pressure. The crude was purified by prep-HPLC (column: Welch Xtimate C18 100*40 mm*3 um; mobile phase: [water(HCl)-ACN];B %: 15%-45%, 10 min) to afford 686 (6.3 mg, 10.18 umol, 5.26% yield) as gray powder.

¹H NMR (400 MHz, DMSO-d₆) δ=8.53-7.71 (m, 1H), 8.55-7.70 (m, 4H), 7.48-7.38 (m, 1H), 7.07-6.61 (m, 3H), 4.74-4.49 (m, 1H), 4.36-4.16 (m, 3H), 4.13-3.84 (m, 4H), 3.64-3.57 (m, 3H), 2.82-2.72 (m, 2H), 2.19-2.13 (m, 5H), 1.81-1.50 (m, 7H), 1.30-1.14 (m, 4H)

LCMS: 100.0%, MS (ESI): m/z 619.2 [M+H]⁺

### Example A-52: Preparation of Macrocyclic Compound 579

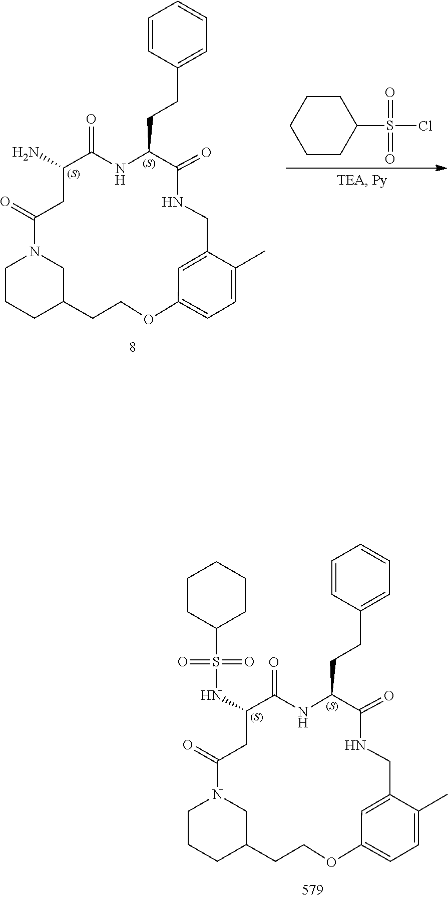

To a solution of 8 (400 mg, 789.52 mmol, 1 eq) in pyridine (Py, 10 mL) was added TEA (239.67 mg, 2.37 mmol, 329.67 mL, 3 eq) and cyclohexane sulfonyl chloride (288.44 mg, 1.58 mmol, 2 eq), the resulting mixture was stirred at 40° C. for 16 hr hours to give yellow solution. LCMS showed the reaction was completed. The resultant solution was partitioned between EA (100 mL) and H₂O (80 mL). The organic layer was washed with water (90 mL×2), brine (90 mL), dried over anhydrous Na₂SO₄, and concentrated under reduced pressure to give a yellow oil. The residue was purified by prep-HPLC (basic condition, column: 2_Phenomenex Gemini C18 75*40 mm*3 um; mobile phase: [water (NH₄HCO₃)-ACN]; B %: 55%-85%, 7.8 min). The afforded flows were combined, concentrated to remove most of CH₃CN, and lyophilized to give 579 (30.3 mg, 46.41 mmol, 50.50% yield) as white solid.

¹H NMR (DMSO-d₆, 400 MHz): δ=7.68-8.68 (m, 2H), 7.16-7.30 (m, 6H), 7.00-7.07 (m, 1H), 6.53-6.99 (m, 2H), 3.56-4.67 (m, 10H), 2.58-3.03 (m, 6H), 2.11-2.21 (m, 4H), 1.05-2.10 ppm (m, 25H).

LCMS: 96.71%, MS (ESI): m/z 651.2 [M+H]⁺.

### Example A-53: Preparation of Macrocyclic Compound 674

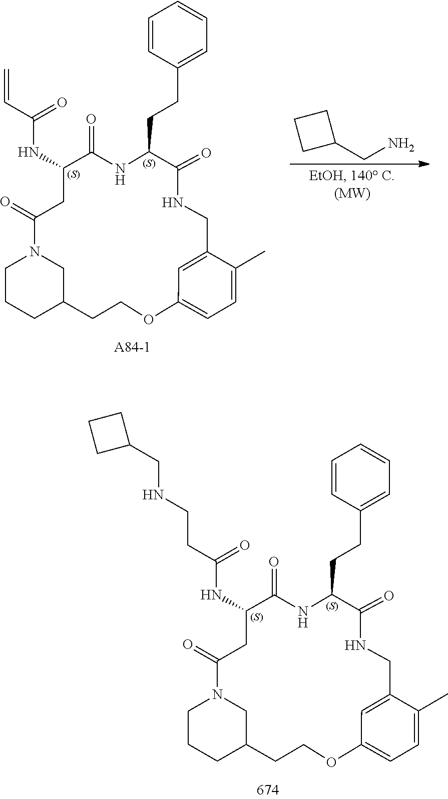

To a solution of A84-1(80 mg, 0.142 mmol, 1 eq) and cyclobutylmethanamine (121.49 mg, 1.43 mmol, 10 eq) in EtOH (3 mL) in a sealed tube and heated to 140° C. with microwave equipment for 30 min to give a yellow solution. LCMS showed a main peak. The reaction solution was purified directly. The residue was purified by prep-HPLC (formic acid (FA) condition, column: Welch Xtimate C18 100*25 mm*3 um; mobile phase: [water(FA)-ACN]; B %: 15%-45%, 8 min) to give 674 (65.1 mg, 0.094 mmol, 65.95% yield, FA) as white solid.

¹H NMR (DMSO-d₆, 400 MHz): δ=7.81-8.60 (m, 4H), 7.14-7.33 (m, 5H), 6.82-7.08 (m, 1H), 6.54-6.81 (m, 2H), 4.41-4.76 (m, 2H), 3.84-4.37 (m, 10H), 2.54-3.03 (m, 10H), 2.06-2.24 (m, 4H), 1.10-2.00 ppm (m, 16H).

LCMS: 95.75%, MS (ESI): m/z 646.4 [M+H]⁺.

### Example A-54: Preparation of Macrocyclic Compound 702

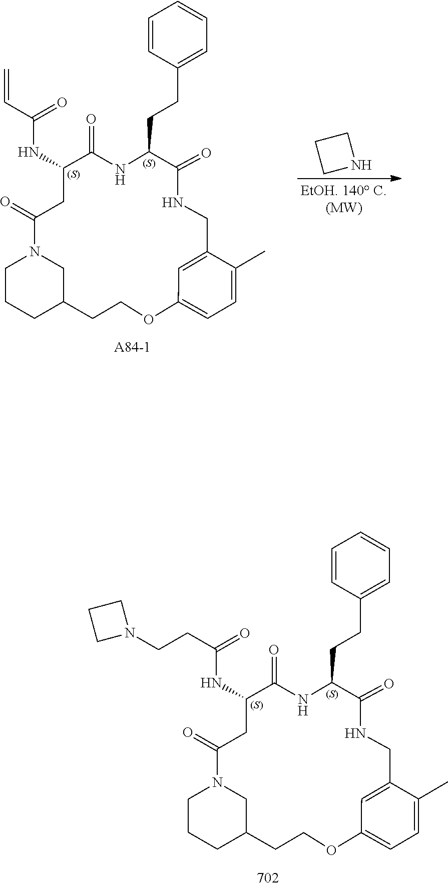

To a solution of A84-1(100 mg, 0.178 mmol, 1 eq) and azetidine (101.83 mg, 1.78 mmol, 0.120 mL, 10 eq) in EtOH (3 mL) in a sealed tube and heated to 140° C. with microwave equipment for 30 min to give a yellow solution. LCMS showed a desired mass. The reaction solution was purified directly. The residue was purified by prep-HPLC (formic acid (FA) condition, column: Welch Xtimate C18 100*25 mm*3 um; mobile phase: [water (FA)-ACN]; B %: 15%-45%, 8 min). The afforded flows were combined, concentrated to remove most of CH₃CN, and lyophilized to 702 (61.2 mg, 0.092 mmol, 51.69% yield, FA) as white solid.

¹H NMR (DMSO-d₆, 400 MHz): δ=8.15-8.45 (m, 3H), 7.11-7.32 (m, 5H), 6.99-7.07 (m, 1H), 6.52-6.96 (m, 2H), 3.80-4.68 (m, 13H), 3.07-3.31 (m, 3H), 2.57-2.87 (m, 6H), 2.04-2.24 (m, 6H), 1.13-2.00 ppm (m, 9H).

LCMS: 98.22%, MS (ESI): m/z 618.2 [M+H]⁺.

### Example A-55: Preparation of Macrocyclic Compound 728

### 1. Preparation of A152-1:

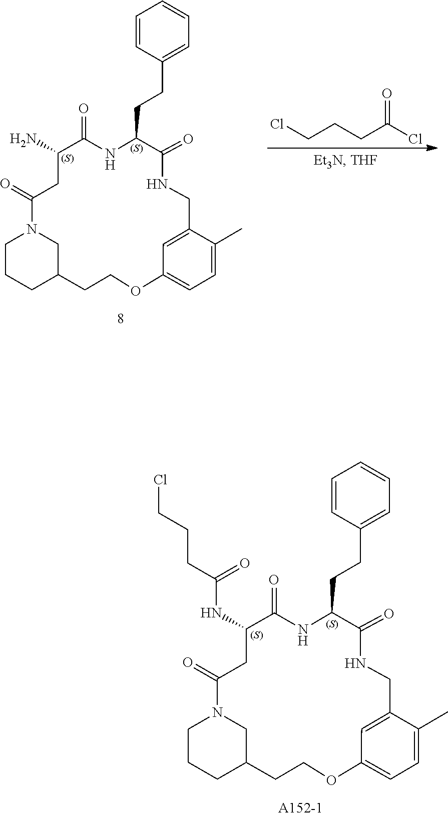

To a solution of 8 (100 mg, 0.184 mmol, 1 eq, HCl) and Et₃N (37.26 mg, 0.368 mmol, 0.051 mL, 2 eq) in THF (5 mL) was added 4-chlorobutanoyl chloride (31.15 mg, 0.220 mmol, 0.024 mL, 1.2 eq) at 0° C. The mixture was stirred at 25° C. for 2 h. The mixture was poured into H₂O (5 mL) and extracted with EA (5 mL×2). The organic was dried with Na₂SO₄ and concentrated under reduced pressure. A152-1(175.9 mg, crude) was obtained without purification as colorless oil.

### 2. Preparation of Compound 728:

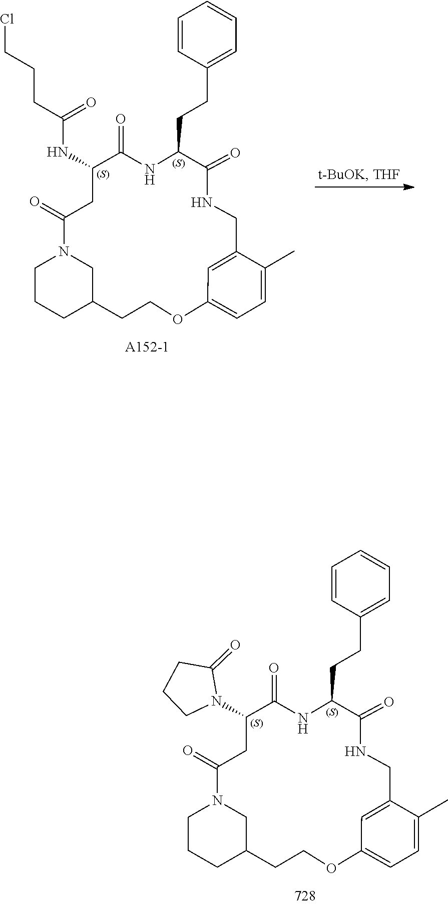

To a solution of A152-1(112.5 mg, 0.184 mmol, 1 eq) in THF (7 mL) was added t-BuOK (103.28 mg, 0.920 mmol, 5 eq). The mixture was stirred at 20° C. for 2 h. The mixture was concentrated under reduced pressure. The crude product was purified by prep-HPLC (column: 2_Phenomenex Gemini C18 75*40 mm*3 um; mobile phase: [water (NH₄HCO₃)-ACN]; B %: 45%-75%, 7.8 min) to afford 728 (27.52 mg, 0.047 mmol, 26.01% yield) as white solid.

¹H NMR (400 MHz, DMSO-d₆) δ=8.43-7.71 (m, 3H), 7.31-7.02 (m, 6H), 6.95-6.72 (m, 2H), 5.16-4.53 (m, 2H), 4.48-4.34 (m, 1H), 4.31-3.96 (m, 5H), 3.91-3.59 (m, 1H), 3.46-3.35 (m, 3H), 3.06-2.57 (m, 5H), 2.18-1.86 (m, 2H), 1.82-1.53 (m, 5H), 1.36-1.18 (m, 2H), 1.10-0.98 (m, 6H)

LCMS: 100.0%, MS (ESI): m/z 575.2 [M+H]⁺

### Example A-56: Preparation of Macrocyclic Compound 602

### 1. Preparation of Compound A63-2:

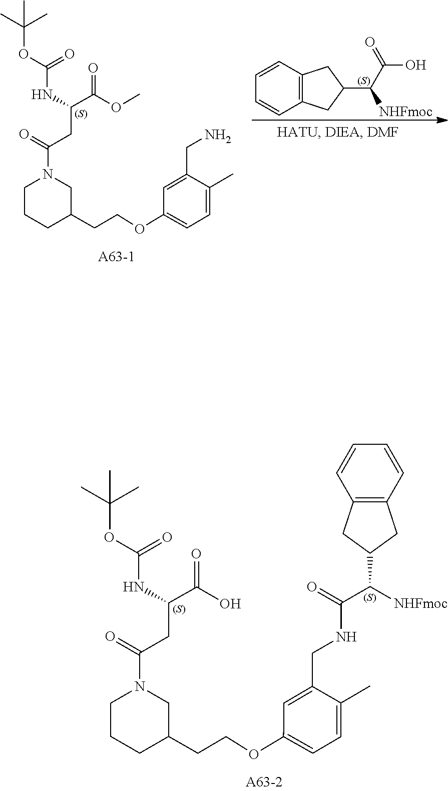

To a solution of A63-1(500 mg, 836.54 umol, 1 eq), (2S)-2-(9H-fluoren-9-ylmethoxycarbonylamino)-2-indan-2-yl-acetic acid (345.88 mg, 836.54 umol, 1 eq), HATU (381.69 mg, 1.00 mmol, 1.2 eq) in DCM (4 mL) and DMF (4 mL) was added DIEA (432.46 mg, 3.35 mmol, 582.83 uL, 4 eq) at 0° C. The reaction was stirred at 0-10° C. for 30 min to give a yellow mixture. LCMS showed that the starting material was consumed completely. H₂O (10 mL) was added. The mixture was concentrated under reduced pressure to give a residue. The solution was partitioned between EA (30 mL) and H₂O (10 mL). The organic layer was washed with 4% lithium chloride solution (30 mL×2), brine (30 mL), dried over anhydrous Na₂SO₄, filtered and concentrated under reduced pressure to give yellow oil. The crude product was purified by silica gel chromatography eluted (EA:PE=0:1 to 2:3) to give A63-2 (457.17 mg, 513.18 umol, 61.35% yield, 98% purity) as yellow oil.

### 2. Preparation of Compound A63-3:

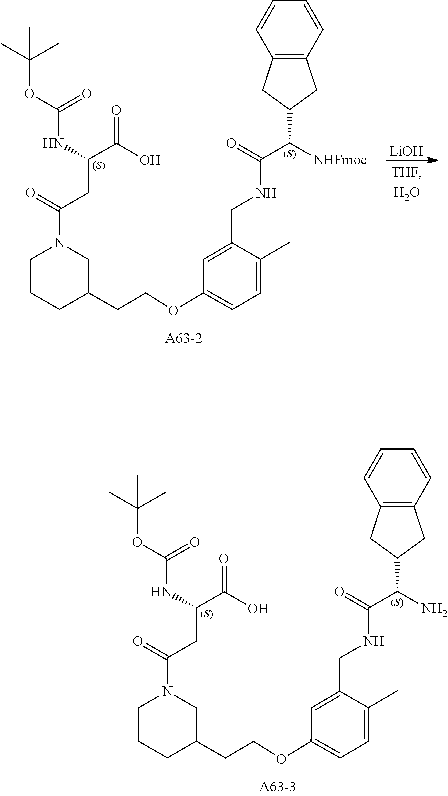

to a solution of A63-2 (417.7 mg, 478.44 umol, 1 eq) in LiOH·H₂O (60.23 mg, 1.44 mmol, 3 eq) was added H₂O (1 mL) in THF (4 mL). The reaction was stirred at 20° C. for 1 h to give a yellow mixture. LCMS showed the starting material was consumed completely. H₂O (60 mL) was added. Then the aqueous layer was acidified to pH=4 by addition of 1 M HCl. The aqueous layer was extracted with EA (60 mL×2). The combined organic layer was washed with brine (60 mL), dried over anhydrous Na₂SO₄, filtered and concentrated in vacuum to give A63-3 (290 mg, 455.42 umol, 95.19% yield) as yellow oil. The crude product was used for the next step without purification.

### 3. Preparation of Compound A63-4:

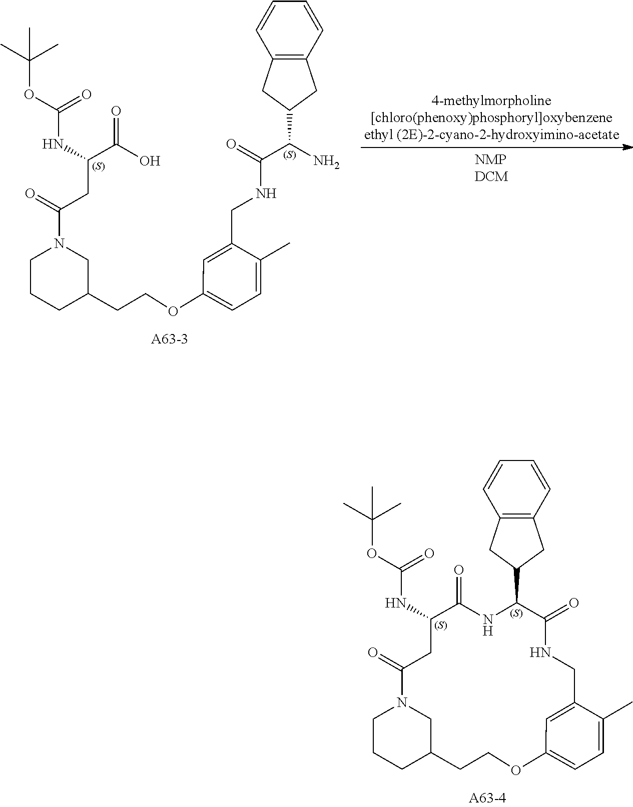

To a solution of ethyl (2E)-2-cyano-2-hydroxyimino-acetate (129.44 mg, 910.84 umol, 2 eq), [chloro(phenoxy)phosphoryl]oxybenzene (244.68 mg, 910.84 umol, 188.21 uL, 2 eq), NMM (276.39 mg, 2.73 mmol, 300.43 uL, 6 eq) in NMP (2 mL) and DCM (4 mL) was A63-3 (290 mg, 455.42 umol, 1 eq). The reaction was stirred at 20° C. for 1.5 h to give a yellow solution. LCMS showed that the reaction was completed. The resultant solution was partitioned between H₂O (100 mL) and EA (120 mL×2). The organic layer was washed with water (200 mL×3), brine (60 mL), dried over anhydrous Na₂SO₄, concentrated under reduced pressure to give a yellow oil. The crude product was purified by silica gel chromatography eluted (EA:PE=0:1 to 2:3) to give A63-4 (126.3 mg, 204.12 umol, 44.82% yield) as yellow oil.

### 4. Preparation of Compound A63-5:

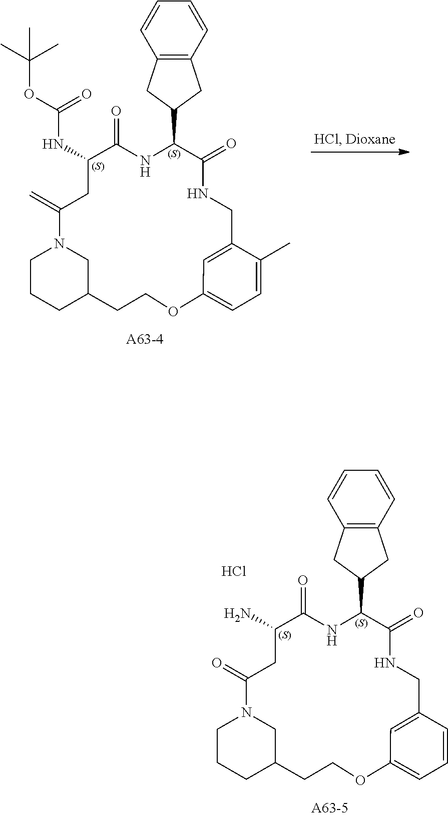

To a solution of A63-4 (126.3 mg, 204.12 umol, 1 eq) in dioxane (2 mL) was added HCl/dioxane (4 M, 153.09 uL, 3 eq) The reaction was stirred at 20° C. for 30 min to give a yellow solution. LCMS showed the starting material was consumed completely. The solution was concentrated under reduced pressure to give A63-5 (94 mg, crude, HCl) as yellow oil. The crude product was used for the next step without purification.

### 5. Preparation of Compound 602:

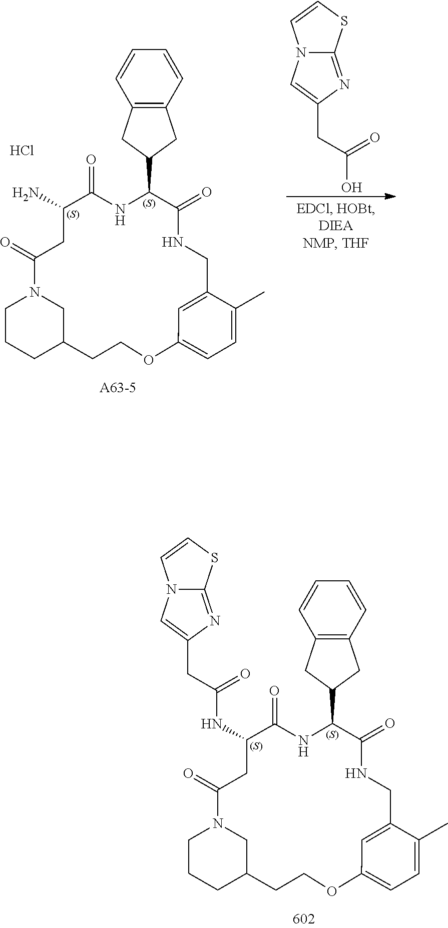

A solution of A63-5 (60 mg, 108.09 umol, 1 eq, HCl), 2-imidazo[2,1-b]thiazol-6-ylacetic acid (19.69 mg, 108.09 umol, 1 eq), DIEA (41.91 mg, 324.26 umol, 56.48 uL, 3 eq), HOBt (18.99 mg, 140.51 umol, 1.3 eq) in THF (2 mL) and DCM (2 mL) was stirred at 20° C. for 15 min to give a yellow mixture. EDCl (24.86 mg, 129.70 umol, 1.2 eq) were added. The reaction was stirred at 20° C. for 1 h to give a yellow mixture. LCMS showed the starting material was consumed not completely. 2-imidazo[2,1-b]thiazol-6-ylacetic acid (19.69 mg, 108.09 umol, 1 eq), DIEA (41.91 mg, 324.26 umol, 56.48 uL, 3 eq), EDCl (24.86 mg, 129.70 umol, 1.2 eq), HOBt (18.99 mg, 140.51 umol, 1.3 eq) was added. The reaction was stirred at 20° C. for 1 h to give a yellow mixture. LCMS showed the starting material was consumed completely. The solution was partitioned between EA (30 mL) and H₂O (15 mL). The organic layer was washed with 0.2 M HCl (30 mL), sat. NaHCO₃ (30 mL), brine (30 mL), dried over anhydrous Na₂SO₄, concentrated under reduced pressure to give yellow oil. The crude product was purified by prep-HPLC (column: 2_Phenomenex Gemini C18 75*40 mm*3 um; mobile phase: [water(NH₄HCO₃)-ACN];B %: 38%-68%, 9.5 min). The eluent was lyophilized to give 602 (2 mg, 2.78 umol, 2.57% yield, 95% purity) as off-white powder.

¹H NMR (400 MHz, DMSO-d₆) δ ppm 7.53-8.58 (m, 4H) 7.26-7.27 (m, 1H) 6.58-7.30 (m, 6H) 3.57-4.75 (m, 9H) 2.70-3.21 (m, 8H) 1.08-2.13 (m, 12H)

LCMS: 95.17%, MS (ESI): m/z 683.2 [M+H]⁺.

### Example A-57: Preparation of Macrocyclic Compound 731

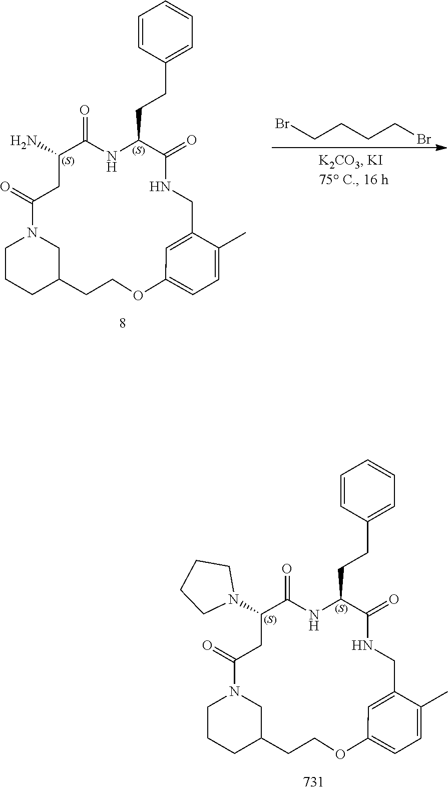

To a solution of 8 (80 mg, 0.157 mmol, 1 eq) in AcCN (5 mL) was added K₂CO₃ (21.82 mg, 0.157 mmol, 1 eq) and KI (1.31 mg, 0.0079 mmol, 0.05 eq), then the mixture was combined with 1, 4-dibromobutane (34.09 mg, 0.157 mmol, 0.019 mL, 1 eq) and stirred at 75° C. for 14 h to give a yellow solution. The reaction was diluted with water (10 ml) and extracted with EtOAc (20 ml×2). The combined organic layers were dried over Na₂SO₄ and concentrated under reduced pressure. The crude product was purified by Prep-HPLC (NH₄Cl) to obtained 731 (11.67 mg, 0.020 mmol, 13.18% yield) as white solid.

¹H NMR (400 MHz, DMSO-d₆) δ ppm 1.05-1.85 (m, 13H) 1.97-2.36 (m, 5H) 2.53-2.59 (m, 3H) 2.60-2.89 (m, 6H) 3.36-3.50 (m, 1H) 3.77-4.59 (m, 7H) 6.69-7.31 (m, 8H) 7.69-8.50 (m, 2H)

LCMS: 100.00%, ESI-MS (m/z): m/z 561.2 [M+H⁺]

### Example A-58: Preparation of Macrocyclic Compound 604

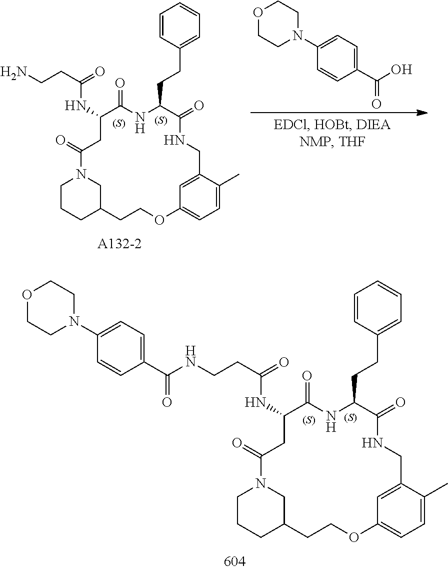

To a solution of A132-2 (120 mg, 0.195 mmol, 1 eq, HCl) and 4-morpholinobenzoic acid (60.73 mg, 0.293 mmol, 1.5 eq) in NMP (1 mL) and THF (4 mL) was added HOBt (39.60 mg, 0.293 mmol, 1.5 eq) and DIEA (101.01 mg, 0.781 mmol, 0.136 mL, 4 eq) stirred for 20 min. Added EDCl (56.18 mg, 0.293 mmol, 1.5 eq) to the reaction. The mixture was stirred at 20° C. for 1 hr to give a colorless solution. LCMS showed the starting material was consumed completely. The reaction mixture was diluted with H₂O (20 mL) and extracted with EA (15 mL×3). The combined organic layers were washed with brine (30 mL×2), dried over Na₂SO₄, filtered and concentrated under reduced pressure to give a residue. The residue was purified by prep-HPLC (column: 2_Phenomenex Gemini C18 75*40 mm*3 um; mobile phase: [water(NH₄HCO₃)-ACN];B %: 43%-73%, 7.8 min) afforded flows were combined, concentrated to remove most of CH₃CN and lyophilized to give 604 (25.9 mg, 0.033 mmol, 17.28% yield, 100% purity) as white powder.

¹H NMR (400 MHz, DMSO-d₆) δ ppm 8.11-8.53 (3H, m) 7.69-7.91 (2H, m) 7.10-7.29 (5H, m) 6.86-7.05 (3H, m) 6.64-6.77 (1H, m) 3.70-4.78 (13H, m) 3.17 (3H, br d, J=4.17 Hz) 2.54-2.99 (7H, m) 2.29-2.48 (3H, m) 2.09-2.19 (3H, m) 1.14-2.01 (9H, m)

LCMS: 100%, MS (ESI): m/z 767.6 [M+H]⁺

### Example A-59: Preparation of Macrocyclic Compound 696

### 1. Preparation pf A43-4:

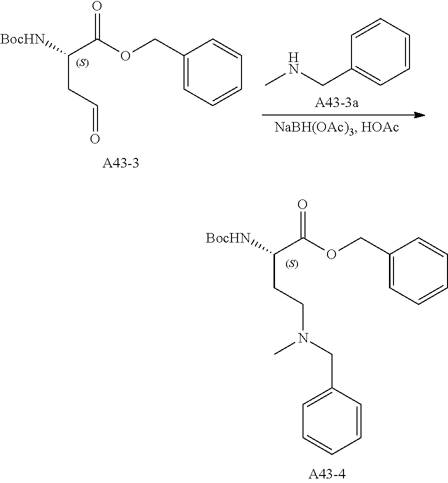

To a solution of A43-3 (1 g, 3.25 mmol, 1 eq) in DCM (10 mL) was added A43-3a (788.57 mg, 6.51 mmol, 839.79 uL, 2 eq), HOAc (acetic acid, 39.08 mg, 650.74 umol, 37.22 uL, 0.2 eq). Then 30 min after was added NaBH(OAc)₃ (1.38 g, 6.51 mmol, 2 eq). The mixture was stirred at 20° C. for 16 hrs to give a yellow solution. The reaction mixture was poured into 0.2M HCl (10 ml) and extracted with EtOAc (10 mL×4). The combined organic layers were washed with brine (20 mL), dried over Na₂SO₄, filtered and concentrated under reduced pressure. The crude product was purified by flash column (SiO₂, PE to 50% EtOAc in PE) to afford A43-4 (620 mg, 1.11 mmol, 34.09% yield, 73.8% purity) was obtained as yellow oil.

### 2. Preparation of A43-5:

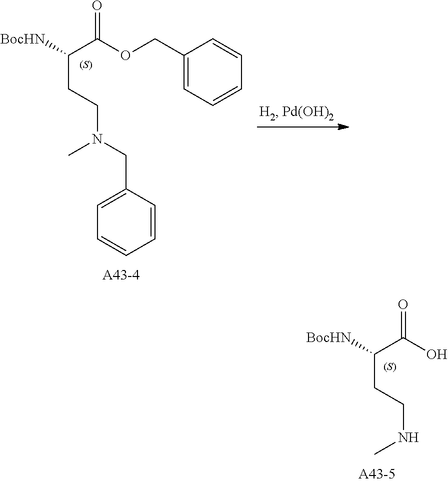

To a solution of A43-5 (620 mg, 1.50 mmol, 1 eq) in MeOH (10 mL) was added Pd(OH)₂ (1.06 g, 1.50 mmol, 20% purity, 1 eq) under H₂ (15 Psi), the resulting mixture was stirred at 20° C. for 14 hours to give yellow solution. The reaction mixture was removed by filtration and the filtrate was concentrated under vacuum to afford A43-5 (349 mg, crude) was obtained as white oil.

### 3. Preparation of A43-7:

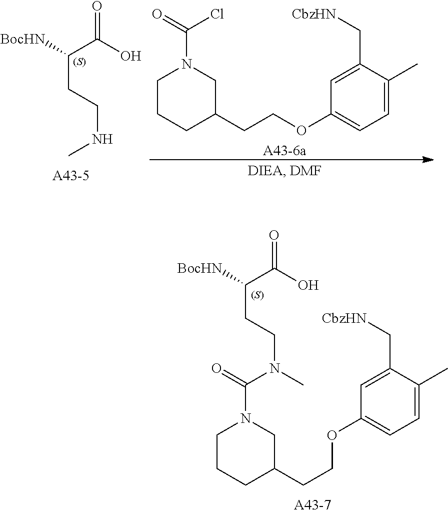

To a solution of A43-5 (340 mg, 1.46 mmol, 1 eq), A43-6a (651.31 mg, 1.46 mmol, 1 eq), DIEA (103.79 mg, 803.08 umol, 139.88 uL, 1.2 eq) in NMP (3 mL) was added DIEA (567.55 mg, 4.39 mmol, 764.89 mL, 3 eq). The mixture was stirred at 20° C. for 2 hr to give a yellow solution. The reaction mixture was poured into 1M HCl(10 mL) and water (50 mL) and extracted with EtOAc (30 mL×4). The combined organic layers were washed with brine (20 mL), dried over Na₂SO₄, filtered and concentrated under reduced pressure to afford A43-7 (1.1 g, crude) was obtained as white oil.

### 4. Preparation of A43-8:

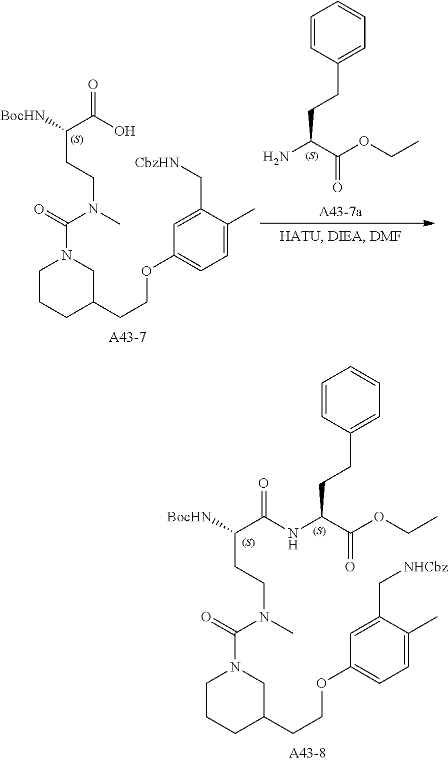

To a solution of A43-7 (1.1 g, 0.546 mmol, 31.5% purity, 1 eq), A43-7a (131.80 mg, 0.546 mmol, 1 eq, HCl), DIEA (209.67 mg, 1.62 mmol, 0.282 mL, 3 eq) in DMF (2 mL) and DCM (4 mL) was added HATU (411.23 mg, 1.08 mmol, 2 eq) at 0° C. The reaction was stirred at 0-10° C. for 2 h to give a yellow solution. The reaction mixture was concentrated directly. The reaction mixture was poured into water (10 mL) and extracted with EtOAc (10 mL×4). The combined organic layers were washed with brine (20 mL), dried over Na₂SO₄, filtered and concentrated under reduced pressure. The crude product was purified by flash column (SiO2, PE to 50% EtOAc in PE) to afford A43-8 (240 mg, 0.289 mmol, 53.47% yield, 100% purity) was obtained as white solid.

### 5. Preparation of A43-9:

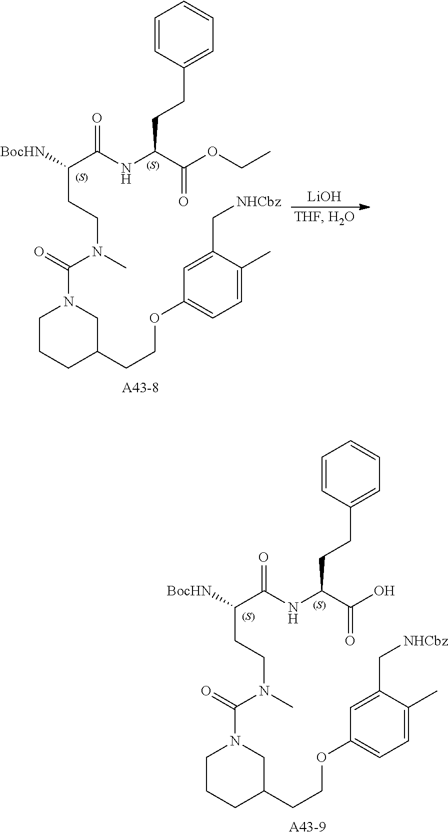

To a solution of A43-8 (240 mg, 0.289 mmol, 1 eq) in H₂O (1 mL) and THF (4 mL) was added LiOH·H₂O (24.27 mg, 0.578 mmol, 2 eq), the resulting mixture was stirred at 20° C. for 4 hours to give yellow solution. The reaction mixture was concentrated directly. The reaction was poured into H₂O (4 mL). The afforded water layer was acidified with 1 N HCl aq. to pH=3, extracted with DCM (100 mL×3). The combined organic layers were washed with brine (10 mL), dried over Na₂SO₄, filtered and concentrated under reduced pressure to afford A43-9 (180 mg, crude) was obtained as white solid.

### 6. Preparation of A43-10:

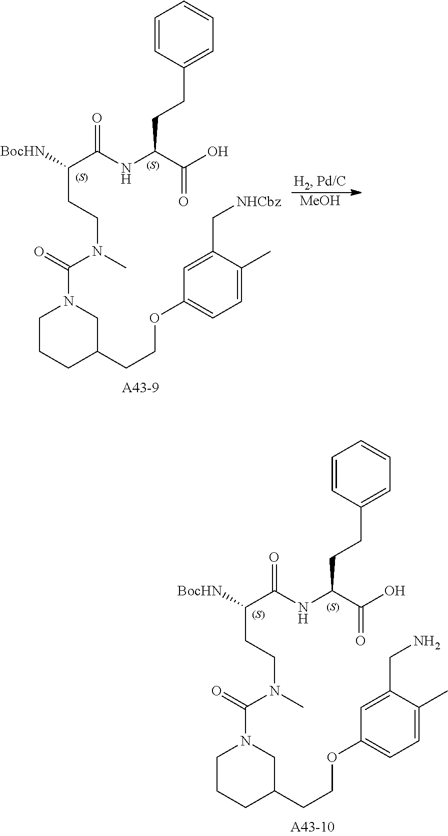

To a solution of A43-10 (180 mg, 0.224 mmol, 1 eq) in MeOH (5 mL) was added Pd/C (50 mg, 0.224 mmol, 10% purity, 1 eq) under H₂ (15 Psi), the resulting mixture was stirred at 20° C. for 14 hours to give yellow solution. The reaction mixture was removed by filtration and the filtrate was concentrated under vacuum to afford A43-10 (149 mg, crude) was obtained as yellow oil.

### 7. Preparation of A43-11:

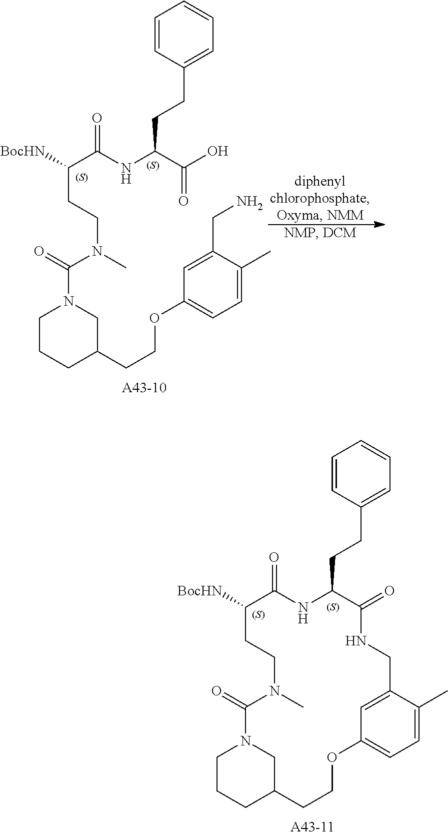

To a solution of Oxyma (63.41 mg, 0.446 mmol, 2 eq), DPCP (diphenyl chlorophosphate) (119.87 mg, 0.446 mmol, 92.21 uL, 2 eq), NMM (N-methylmorpholine) (135.40 mg, 1.34 mmol, 147.18 uL, 6 eq) in NMP (N-methylpyrrolidone) (2 mL) and DCM (5 mL) was added A43-10 (149 mg, 0.223 mmol, 1 eq) and the reaction was stirred at 20° C. for 2 h to give a yellow solution. The resultant solution was concentrated to remove DCM. The reaction mixture was poured into water (10 mL) and extracted with EtOAc (10 mL×4). The combined organic layers were washed with brine (20 mL), dried over Na₂SO₄, filtered and concentrated under reduced pressure. The crude product was purified by flash column (SiO2, PE to 90% EtOAc in PE) to afford A43-11 (70 mg, 0.107 mmol, 48.28% yield) was obtained as yellow oil.

### 8. Preparation of A43-12:

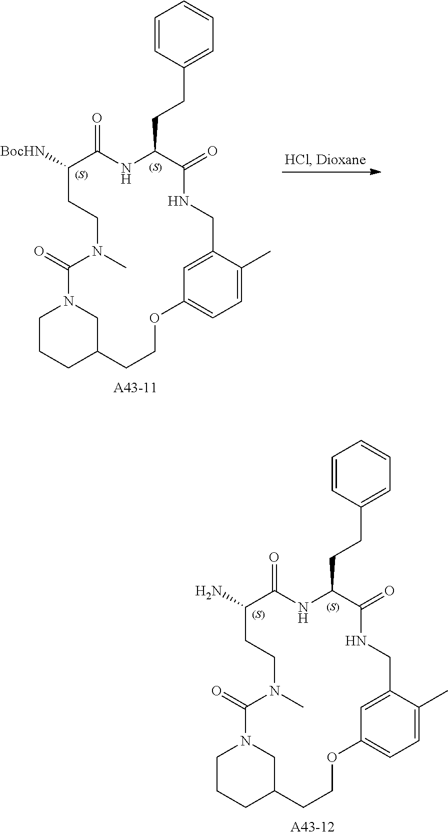

To a solution of A43-12 (70 mg, 0.107 mmol, 1 eq) in HCl/dioxane (1 mL), the resulting mixture was stirred at 0° C. for 4 hours to give yellow mixture. The reaction mixture was concentrated directly to afford A43-12 (63 mg, crude, HCl) was obtained as yellow oil.

### 9. Preparation of Compound 696:

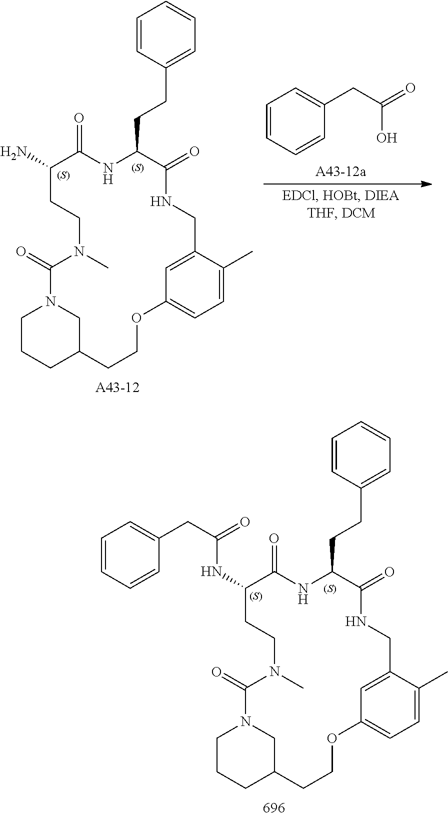

To a solution of A43-5 (50 mg, 0.091 mmol, 1 eq, HCl), A43-5a (21.57 mg, 0.118 mmol, 1.3 eq), HOBt (17.22 mg, 0.127 mmol, 1.4 eq), DIEA (35.30 mg, 0.273 mmol, 0.047 mL, 3eq) in THF (2 mL) and DCM (2 mL) was stirred at 20° C. for 15 min to give a yellow mixture. EDC HCl (24.44 mg, 0.127 mmol, 1.4 eq) were added. the reaction was stirred at 20° C. for 1 h to give a yellow mixture. The reaction mixture was poured into water (10 mL) and extracted with EtOAc (10 mL×4). The organic layer was washed with 0.2 M HCl (20 mL), sat. NaHCO₃ (30 mL), brine (30 mL), dried over anhydrous Na₂SO₄, concentrated under reduced pressure to give yellow oil. The residue was purified by prep-HPLC (neutral, column: 2_Phenomenex Gemini C18 75*40 mm*3 um; mobile phase: [water (NH₄HCO₃)-ACN]; B %: 52%-82%, 7.8 min). The afforded flows were combined, concentrated to remove most of CH₃CN and lyophilized to afford 696 (12.40 mg, 0.018 mmol, 18.14% yield, 100% purity) was obtained as white solid.

¹H NMR (400 MHz, DMSO-d₆) δ=8.46-8.00 (m, 3H), 7.34-6.59 (m, 14H), 4.33-3.43 (m, 4H), 3.28-2.55 (m, 1H), 2.16 (d, J=3.0 Hz, 3H), 2.07-1.07 (m, 13H)

LCMS: 100%, MS (ESI): m/z 668.3 [M+H]⁺.

### Example A-60: Preparation of Macrocyclic Compound 651

### 1. Preparation of A141-2a:

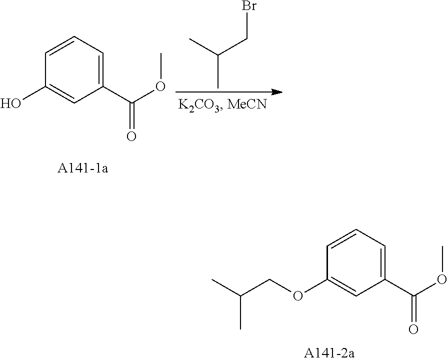

To a solution of A141-1a (300 mg, 1.97 mmol, 1 eq) in DMF (3.5 mL) was added 1-bromo-2-methyl-propane (405.25 mg, 2.96 mmol, 0.321 m, 1.5 eq) and K₂CO₃ (817.53 mg, 5.92 mmol, 3 eq), the mixture stirred at 80° C. for 3h to give a brown mixture. TLC showed the starting material was consumed completely. The mixture was partitioned between EA (10 mL×2) and H₂O (10 mL). The organic layer was washed with H₂O (10 mL×3), brine (20 mL), dried over anhydrous Na₂SO₄, filtered and concentrated under reduced pressure to give a residue. The crude product was purified by flash column (SiO₂, 100% PE) to give A141-2a (340 mg, crude) as brown oil.

### 2. Preparation of A141-3a:

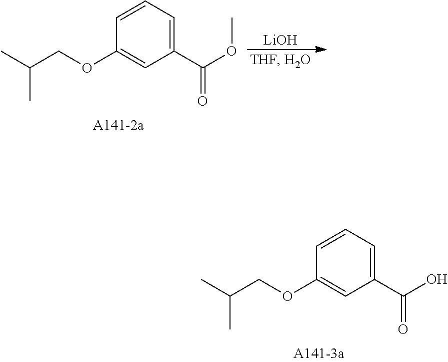

To a solution of A141-2a (260 mg, 1.25 mmol, 1 eq) in THF (3 mL) and H₂O (0.5 mL) was added LiOH·H₂O (157.17 mg, 3.75 mmol, 3 eq) at 0° C. The mixture was stirred at 20° C. for 3 h to give yellow mixture. TLC showed the starting material was consumed completely. The reaction was diluted with EA (10 mL) and neutralized with 1 N HCl. The combined organic layers were washed with brine (10 mL), dried over Na₂SO₄, filtered and concentrated under reduced pressure to give A141-3a (150 mg, crude) was obtained as colorless oil.

### 3. Preparation of Compound 651:

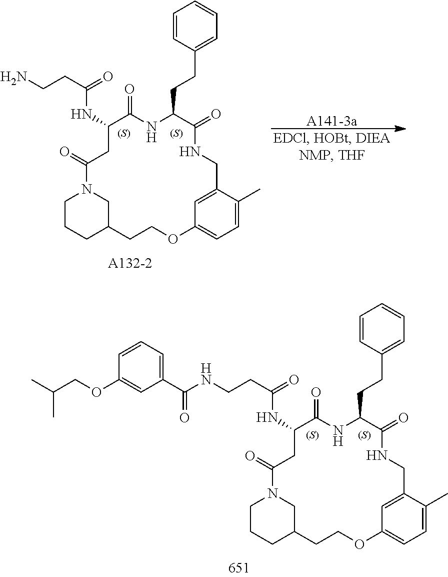

To a solution of A132-2 (100 mg, 0.162 mmol, 1 eq, HCl) and A141-3a (47.44 mg, 0.244 mmol, 1.5 eq) in NMP (0.8 mL) and THF (3 mL) was added HOBt (33.00 mg, 0.244 mmol, 1.5 eq) and DIEA (84.17 mg, 0.651 mmol, 0.113 mL, 4 eq) stirred for 20 min. Added EDCl (46.82 mg, 0.244 mmol, 1.5 eq) to the reaction. The mixture was stirred at 20° C. for 1 hr to give a colorless solution. LCMS showed the starting material was consumed completely. The reaction mixture was diluted with H₂O (20 mL) and extracted with EA (15 mL×3). The combined organic layers were washed with brine (30 mL×2), dried over Na₂SO₄, filtered and concentrated under reduced pressure to give a residue. The residue was purified by prep-HPLC (column: 2_Phenomenex Gemini C18 75*40 mm*3 um; mobile phase: [water(NH₄HCO₃)-ACN];B %: 55%-85%, 7.8 min). The afforded flows were combined, concentrated to remove most of CH₃CN and lyophilized to give 651 (28.08 mg, 0.037 mmol, 22.87% yield, 100% purity) as white powder

¹H NMR (400 MHz, DMSO-d₆) δ ppm 7.78-8.51 (4H, m) 7.10-7.43 (8H, m) 6.60-7.08 (2H, m) 3.41-4.81 (12H, m) 2.65-2.96 (3H, m) 2.28-2.40 (3H, m) 2.12-2.18 (3H, m) 1.08-2.08 (12H, m) 0.97 (6H, br d, J=6.27 Hz)

LCMS: 100%, MS (ESI): m/z 754.2 [M+H]⁺

### Example A-61: The Following Compounds have been Synthesized According to the Above-Described Methods

LCMS was taken on a quadrupole Mass Spectrometer on Shimadzu LCMS 2010 (Shim-pack XR-ODS 3.0*30 mm 2.2 μm) operating in ES (+) ionization mode. Flow Rate: 0.8 mL/min, Acquire Time: 3 min, Wavelength: UV220, Oven Temp.: 50° C.

TABLE A-43

| Cpd | Chemical | Exact | [M + H]⁺ | Purity |
|---|---|---|---|---|
| No. | Formula | mass | found | (%) |
| 573 | C34H46N4O6 | 606.3 | 607.6 | 100 |
| 574 | C43H54N6O8 | 782.4 | 783.8 | 96.9 |
| 575 | C36H48N6O5S | 676.3 | 677.2 | 100 |
| 576 | C43H59N5O7 | 757.4 | 758.3 | 100.0 |
| 577 | C43H59N5O8 | 773.4 | 774.9 | 99.8 |
| 578 | C42H52N6O8 | 768.4 | 769.4 | 98.1 |
| 580 | C41H55N5O7 | 729.4 | 730.2 | 100 |
| 581 | C41H52N4O7 | 712.4 | 713.3 | 97.7 |
| 582 | C41H52N4O7 | 712.4 | 713.3 | 99.2 |
| 583 | C33H44N6O5S | 636.3 | 637.3 | 99.8 |
| 584 | C40H54N4O6 | 686.4 | 687.3 | 100 |
| 585 | C40H50N4O7 | 698.4 | 699.3 | 100.0 |
| 586 | C40H45N5O5 | 675.3 | 676.4 | 100 |
| 587 | C39H48N4O7 | 684.4 | 685.3 | 100.0 |
| 588 | C39H48N4O7 | 684.4 | 685.2 | 98.4 |
| 589 | C39H48N4O6 | 668.4 | 669.3 | 97.8 |
| 590 | C38H50N4O7 | 674.4 | 675.2 | 100.0 |
| 591 | C38H46N4O6 | 654.3 | 655.2 | 100.0 |
| 592 | C37H50FN5O5 | 663.4 | 664.4 | 100 |
| 594 | C36H48N4O6 | 632.4 | 633.3 | 100.0 |
| 595 | C36H48N4O6 | 632.4 | 633.3 | 100.0 |
| 596 | C35H48N4O7 | 636.4 | 637.3 | 100.0 |
| 597 | C35H46N4O6 | 618.3 | 619.2 | 100.0 |
| 598 | C32H41F2N5O5 | 613.3 | 614.3 | 90 |
| 599 | C32H40F2N4O6 | 614.3 | 615.5 | 100 |
| 600 | C36H42FN5O5 | 643.3 | 644.4 | 100 |
| 601 | C31H39F3N4O6S | 652.3 | 653.2 | 100 |
| 603 | C44H52N6O5 | 744.4 | 745.3 | 98.5 |
| 605 | C42H57N5O7 | 743.4 | 744.3 | 100 |
| 606 | C42H54N4O7 | 726.4 | 727.3 | 100 |
| 607 | C42H54N4O7 | 726.4 | 727.3 | 98.7 |
| 608 | C42H59N5O5 | 713.5 | 714.3 | 100 |
| 609 | C42H49N5O5 | 703.4 | 704.5 | 100 |
| 610 | C41H52N4O7 | 712.4 | 713.3 | 99.8 |
| 611 | C41H57N5O6 | 715.4 | 716.3 | 100 |
| 612 | C41H52N4O6 | 696.4 | 697.3 | 100 |
| 613 | C41H57N5O5 | 699.4 | 700.4 | 99.6 |
| 614 | C40H54N4O6 | 686.4 | 687.3 | 98.4 |
| 615 | C40H54N4O6 | 686.4 | 687.3 | 100 |
| 616 | C40H53N5O6 | 699.4 | 700.3 | 100 |
| 617 | C40H53N5O6 | 699.4 | 700.3 | 99.2 |
| 618 | C40H50N4O6 | 682.4 | 683.3 | 100 |
| 619 | C40H50N4O7 | 698.4 | 699.2 | 100 |
| 620 | C40H56N4O6 | 688.4 | 689.8 | 99.4 |
| 621 | C40H55N5O6 | 701.4 | 702.3 | 97.3 |
| 622 | C39H48N4O7 | 684.4 | 685.2 | 100 |
| 623 | C39H54N4O6 | 674.4 | 675.3 | 100 |
| 625 | C38H52N4O6 | 660.4 | 661.3 | 100 |
| 626 | C38H52N4O6 | 660.4 | 661.5 | 99.1 |
| 627 | C38H52N4O6 | 660.4 | 661.3 | 100 |
| 629 | C37H50N4O6 | 646.4 | 647.3 | 94.8 |
| 630 | C37H50N4O6 | 646.4 | 647.3 | 97.0 |
| 631 | C37H48N4O5 | 628.4 | 629.3 | 100 |
| 633 | C35H43FN4O5 | 618.3 | 619.3 | 100 |
| 634 | C38H47N5O5 | 653.4 | 654.4 | 100 |
| 635 | C34H44N4O6 | 604.3 | 605.2 | 100 |
| 636 | C36H43FN4O6S | 678.3 | 679.3 | 96.5 |
| 637 | C42H54N4O7 | 726.4 | 727.2 | 100 |
| 638 | C40H55N5O5 | 685.4 | 686.4 | 99.7 |
| 639 | C39H53N5O5 | 671.4 | 672.3 | 100 |
| 640 | C39H53N5O6 | 687.4 | 688.3 | 97.8 |
| 641 | C39H53N5O6 | 687.4 | 688.3 | 100 |
| 642 | C39H52N4O6 | 672.4 | 673.3 | 98.6 |
| 643 | C36H48N4O5 | 616.4 | 617.3 | 100 |
| 644 | C34H46N4O6 | 606.3 | 607.5 | 100 |
| 645 | C37H46N6O5 | 654.4 | 655.6 | 100 |
| 646 | C34H43F3N4O5 | 644.3 | 645.3 | 98.1 |
| 647 | C39H49N5O5 | 667.4 | 668.3 | 99.6 |
| 648 | C38H51N5O6 | 673.4 | 674.3 | 100 |
| 649 | C35H47N5O5 | 617.4 | 618.5 | 98.0 |
| 650 | C34H45FN4O5 | 608.3 | 609.4 | 100 |
| 652 | C40H55N5O5 | 685.4 | 686.3 | 95.8 |
| 654 | C37H46N6O5 | 654.4 | 655.2 | 100 |
| 655 | C35H44N4O6 | 616.3 | 617.2 | 94.5 |
| 656 | C45H57N5O6 | 763.4 | 764.3 | 94.8 |
| 657 | C39H53N5O5 | 671.4 | 672.3 | 100 |
| 658 | C39H53N5O6 | 687.4 | 688.3 | 99.0 |
| 659 | C38H51N5O5 | 657.4 | 658.3 | 100 |
| 660 | C37H49N5O6 | 659.4 | 660.5 | 100 |
| 661 | C36H41FN4O5 | 628.3 | 629.2 | 98.0 |
| 662 | C35H45N5O5 | 615.3 | 616.5 | 97.4 |
| 663 | C34H45N5O5 | 603.3 | 604.3 | 100 |
| 664 | C34H44N4O6 | 604.3 | 605.4 | 95.6 |
| 665 | C43H56N6O6 | 752.4 | 753.3 | 100 |
| 666 | C40H50N6O5 | 694.4 | 695.2 | 98.6 |
| 667 | C39H48N6O5 | 680.4 | 681.6 | 96.1 |
| 668 | C36H49N5O5 | 631.4 | 632.5 | 100 |
| 669 | C34H45N5O6 | 619.3 | 620.3 | 100 |
| 670 | C35H46N4O6 | 618.3 | 619.5 | 100 |
| 671 | C36H48N4O7 | 648.4 | 649.5 | 100 |
| 672 | C45H59N5O8S | 829.4 | 830.3 | 100 |
| 673 | C38H51N5O5 | 657.4 | 658.3 | 96.1 |
| 675 | C37H51N5O5 | 645.4 | 646.4 | 99.8 |
| 676 | C36H49N5O6 | 647.4 | 648.3 | 100 |
| 677 | C38H53N5O5 | 659.4 | 660.3 | 99.9 |
| 678 | C37H46N6O5 | 654.4 | 655.4 | 100 |
| 679 | C37H51N5O5 | 645.4 | 646.5 | 93.9 |
| 680 | C36H49N5O5 | 631.4 | 632.3 | 100 |
| 681 | C35H47N5O6 | 633.4 | 634.3 | 95.2 |
| 682 | C34H47N5O5 | 605.4 | 606.4 | 97.4 |
| 683 | C33H45N5O6 | 607.3 | 608.5 | 100 |
| 684 | C33H43FN4O6 | 610.3 | 611.3 | 100 |
| 685 | C32H40N4O5 | 560.3 | 561.2 | 100 |
| 687 | C38H46N6O5 | 666.4 | 667.3 | 96.8 |
| 688 | C36H50N6O5 | 646.4 | 647.3 | 98.3 |
| 689 | C36H48N4O6 | 632.4 | 633.5 | 100 |
| 690 | C34H48N4O5 | 592.4 | 593.3 | 100 |
| 691 | C33H43N5O6 | 605.3 | 606.2 | 100 |
| 692 | C40H56N4O6 | 688.4 | 688.6 | 99.0 |
| 693 | C38H53N5O5 | 659.4 | 660.5 | 99.1 |
| 694 | C36H49N5O5 | 631.4 | 632.3 | 100 |
| 695 | C39H53N5O5 | 671.4 | 672.3 | 100 |
| 697 | C36H47N5O6 | 645.4 | 646.2 | 100 |
| 698 | C41H57N5O5 | 699.4 | 700.7 | 94.1 |
| 699 | C50H63N7O5 | 841.5 | 842.3 | 99.7 |
| 700 | C39H55N5O5 | 673.4 | 674.7 | 94.8 |
| 701 | C39H51N5O7 | 701.4 | 702.2 | 100 |
| 703 | C37H44N6O5 | 652.3 | 653.3 | 97.9 |
| 704 | C38H46N6O5 | 666.4 | 667.4 | 97.4 |
| 705 | C38H45N5O6 | 667.3 | 668.5 | 96.3 |
| 706 | C46H61N5O6 | 779.5 | 780.8 | 97.2 |
| 707 | C37H44N4O6 | 640.3 | 641.4 | 98.5 |
| 708 | C36H43N7O5S | 685.3 | 686.4 | 95.9 |
| 709 | C31H39F3N4O4 | 588.3 | 589.3 | 100 |
| 710 | C39H53N5O6 | 687.4 | 688.6 | 98.0 |
| 712 | C36H48N4O4 | 600.4 | 601.3 | 100 |
| 713 | C38H51N5O5 | 657.4 | 658.6 | 96.5 |
| 714 | C37H49N5O5 | 643.4 | 644.5 | 96.7 |
| 715 | C34H45N5O5 | 603.3 | 604.4 | 94.6 |
| 716 | C34H46N6O5 | 618.4 | 619.3 | 100 |
| 718 | C37H47N5O5 | 641.4 | 642.4 | 100 |
| 719 | C33H43N5O7 | 621.3 | 622.6 | 100 |
| 720 | C38H51N5O6 | 673.4 | 674.5 | 87.7 |
| 721 | C36H47N5O5 | 629.4 | 630.5 | 96.7 |
| 722 | C33H46N4O6 | 594.3 | 595.3 | 100 |
| 723 | C37H49N5O5 | 643.4 | 644.6 | 99 |
| 724 | C34H45N5O7 | 635.3 | 636.6 | 99 |
| 725 | C38H51N5O6 | 673.4 | 674.7 | 99.7 |
| 726 | C32H44N4O6 | 580.3 | 581.3 | 97.6 |
| 727 | C33H47N5O4 | 577.4 | 578.7 | 97.9 |
| 729 | C34H45FN6O5 | 636.3 | 637.4 | 100 |
| 730 | C36H49N5O6 | 647.4 | 648.5 | 100 |
| 732 | C31H42N4O6 | 566.3 | 567.5 | 100 |
| 734 | C34H44N4O5 | 588.3 | 589.2 | 100 |
| 735 | C31H42N4O6 | 566.3 | 567.5 | 100 |
| 736 | C30H40N4O6 | 552.3 | 553.5 | 100 |
| 737 | C36H42FN5O5 | 643.3 | 644.4 | 100 |
| 738 | C38H47N5O5 | 653.4 | 654.5 | 100 |
| 741 | C40H50N4O7 | 698.4 | 699.5 | 99.4 |

### Biological Assays

### Example B-1: β5 Inhibition Assay for the Human Constitutive Proteasome and the Human Immunoproteasome

### Assay Description Constitutive Proteasome and Immunoproteasome

The enzymatic activity of the β5 subunit of the human constitutive proteasome (CPS) is analyzed. In another assay, which differs from the assay of the human constitutive proteasome only in using the human immunoproteasome (IPS) the enzymatic activity of the β5 subunit of the human immunoproteasome (IPS) is analyzed.

Incubation of the enzyme with its fluorogenic substrate Suc-LLVY-AMC leads to liberation of the fluorescent dye AMC which can be measured with a suitable plate reader.

### Reagents/Materials

| | | |
|---|---|---|
| Human cPS | E-360 | Boston Biochem or |
| Human IPS | E-370 | Boston Biochem |
| Suc-LLVY-AMC | I-1955 | Bachem |
| Bortezomib | 349320:01 | Proteros |
| Assay plate | 4514 | Corning |
| Assay Buffer | | |

100 mM HEPES pH7.5
50 mM NaCl
0.02% SDS
1 mM DTT (add fresh)

### Microplate Reader Settings

Victor X5 (Perkin Elmer)
Label: Umbeliferone (0.1s)
Excitation wavelength: 355 nm
Emission wavelength: 460 nm

### Assay Procedure (384 Well Plate)

4 μl human cPS or IPS enzyme in assay buffer (final assay concentration 1 nM) or 4 μl assay buffer for negative controls
Compound addition via acoustic dispenser (Echo 520, Labcyte)
Mix and incubate @ RT for 10 min
4 μl Suc-LLVY-AMC substrate (final assay concentration 22.6 μM)
Mix and incubate @ RT for 4 h (dark)
Stop reaction with 4 μl Bortezomib (final assay concentration 1 μM)
15 min @ RT
Measure (Excitation 355 nm; Emission 460 nm)

Table 2 shows activity data in the Human cPS β5 Inhibition Assay. Inhibition is indicated as IC₅₀ [nM] (“-”=not measured). Compounds having an activity designated as “A” provided an IC₅₀≤100 nM; compounds having an activity designated as “B” provided an 100 nM<IC₅₀≤500 nM; compounds having an activity designated as “C” provided an 500 nM<IC₅₀≤1000 nM; compounds having an activity designated as “D” provided an 1000 nM<IC₅₀≤5000 nM; compounds having an activity designated as “E” provided an an IC₅₀>5000 nM.

TABLE 2

| Cpd | IPS [nM] | CPS [nM] |
|---|---|---|
| 7 | A | A |
| 13 | A | A |
| 19 | A | A |
| 16 | A | A |
| 25 | A | A |
| 26 | A | A |
| 27 | A | A |
| 28 | A | A |
| 29 | A | A |
| 30 | A | A |
| 31 | A | A |
| 32 | A | A |
| 33 | A | A |
| 34 | A | A |
| 35 | A | A |
| 36 | A | A |
| 37 | A | A |
| 39 | A | A |
| 40 | A | A |
| 42 | A | A |
| 43 | A | A |
| 45 | A | A |
| 51 | A | A |
| 8 | A | A |
| 60 | A | A |
| 63 | A | A |
| 64 | A | — |
| 77 | A | A |
| 78 | B | D |
| 79 | A | B |
| 80 | A | A |
| 81 | A | A |
| 82 | A | A |
| 83 | A | A |
| 84 | A | A |
| 85 | A | A |
| 86 | A | A |
| 87 | A | A |
| 88 | A | A |
| 89 | A | A |
| 95 | A | A |
| 116 | B | E |
| 120 | A | A |
| 126 | A | A |
| 127 | A | A |
| 128 | A | A |
| 129 | A | A |
| 130 | A | A |
| 134 | A | A |
| 135 | A | A |
| 136 | A | A |
| 143 | A | A |
| 148 | A | D |
| 152 | A | A |
| 160 | A | A |
| 157 | A | A |
| 158 | A | A |
| 159 | A | A |
| 168 | D | E |
| 171 | A | B |
| 174 | A | A |
| 175 | A | A |
| 176 | A | A |
| 177 | A | A |
| 179 | A | A |
| 180 | A | A |
| 181 | A | A |
| 182 | A | A |
| 186 | A | A |
| 191 | A | C |
| 195 | A | A |
| 199 | A | A |
| 205 | A | A |
| 211 | A | B |
| 213 | A | A |
| 215 | A | A |
| 216 | A | A |
| 217 | A | A |
| 219 | A | A |
| 220 | A | A |
| 258 | A | B |
| 265 | B | — |
| 269 | A | D |
| 281 | D | D |
| 284 | A | A |
| 285 | A | A |
| 286 | A | A |
| 287 | A | A |
| 288 | A | A |
| 289 | B | B |
| 290 | C | D |
| 291 | A | — |
| 293 | A | — |
| 294 | A | A |
| 292 | A | — |
| 282 | C | E |
| 295 | A | A |
| 296 | B | C |
| 299 | C | E |
| 300 | A | B |
| 301 | C | D |
| 283 | A | C |
| 302 | A | A |
| 303 | A | A |
| 304 | A | A |
| 305 | A | A |
| 306 | A | A |
| 307 | A | A |
| 308 | A | A |
| 309 | A | A |
| 310 | A | A |
| 311 | A | A |
| 312 | B | E |
| 313 | C | E |
| 314 | A | A |
| 315 | A | A |
| 316 | A | A |
| 317 | A | A |
| 318 | A | A |
| 319 | A | A |
| 320 | A | A |
| 322 | A | A |
| 323 | A | A |
| 324 | A | A |
| 325 | A | A |
| 326 | A | A |
| 327 | A | A |
| 328 | A | A |
| 329 | A | A |
| 330 | A | A |
| 331 | A | A |
| 332 | A | A |
| 333 | A | A |
| 334 | A | A |
| 335 | A | A |
| 336 | A | A |
| 337 | A | A |
| 338 | A | A |
| 339 | A | A |
| 340 | A | A |
| 341 | A | A |
| 342 | A | A |
| 343 | A | A |
| 344 | A | A |
| 345 | A | A |
| 347 | A | A |
| 348 | A | A |
| 349 | A | A |
| 350 | A | A |
| 352 | A | A |
| 353 | A | A |
| 355 | A | D |
| 356 | A | C |
| 358 | A | B |
| 359 | A | A |
| 360 | A | A |
| 361 | A | A |
| 362 | A | A |
| 363 | A | A |
| 364 | A | A |
| 369 | A | A |
| 371 | A | A |
| 372 | A | A |
| 373 | A | A |
| 503 | A | A |
| 505 | A | A |
| 509 | A | A |
| 510 | A | A |
| 511 | A | A |
| 512 | A | A |
| 513 | A | A |
| 549 | A | A |
| 551 | A | A |
| 552 | A | A |
| 553 | A | A |
| 554 | A | A |
| 555 | A | A |
| 556 | A | A |
| 557 | A | A |
| 558 | A | A |
| 559 | A | A |
| 560 | A | A |
| 561 | A | A |
| 562 | A | A |
| 563 | A | A |
| 564 | A | — |
| 565 | A | — |
| 566 | A | A |
| 567 | A | A |
| 568 | A | A |
| 571 | A | A |
| 573 | A | A |
| 574 | A | A |
| 575 | A | A |
| 576 | A | A |
| 577 | A | A |
| 578 | A | A |
| 579 | A | A |
| 580 | A | A |
| 581 | A | A |
| 582 | A | A |
| 583 | A | A |
| 584 | A | A |
| 585 | A | A |
| 586 | A | A |
| 587 | A | A |
| 588 | A | A |
| 589 | A | A |
| 590 | A | A |
| 591 | A | A |
| 592 | A | A |
| 593 | A | A |
| 594 | A | A |
| 595 | A | A |
| 596 | A | A |
| 597 | A | A |
| 598 | A | A |
| 599 | A | A |
| 600 | A | A |
| 601 | A | A |
| 602 | A | A |
| 603 | A | A |
| 604 | A | A |
| 605 | A | A |
| 606 | A | A |
| 607 | A | A |
| 608 | A | A |
| 609 | A | A |
| 610 | A | A |
| 611 | A | A |
| 612 | A | A |
| 613 | A | A |
| 614 | A | A |
| 615 | A | A |
| 616 | A | A |
| 617 | A | A |
| 618 | A | A |
| 619 | A | A |
| 620 | A | A |
| 621 | A | A |
| 622 | A | A |
| 623 | A | A |
| 624 | A | A |
| 625 | A | A |
| 626 | A | A |
| 627 | A | A |
| 628 | A | A |
| 629 | A | A |
| 630 | A | A |
| 631 | A | A |
| 632 | A | A |
| 633 | A | A |
| 634 | A | A |
| 635 | A | A |
| 636 | A | A |
| 637 | A | A |
| 638 | A | A |
| 639 | A | A |
| 640 | A | A |
| 641 | A | A |
| 642 | A | A |
| 643 | A | A |
| 644 | A | A |
| 645 | A | A |
| 646 | A | A |
| 647 | A | A |
| 648 | A | A |
| 649 | A | A |
| 650 | A | A |
| 651 | A | A |
| 652 | A | A |
| 653 | A | A |
| 654 | A | A |
| 655 | A | A |
| 656 | A | A |
| 657 | A | A |
| 658 | A | A |
| 659 | A | A |
| 660 | A | A |
| 661 | A | A |
| 662 | A | A |
| 663 | A | A |
| 664 | A | A |
| 665 | A | A |
| 666 | A | A |
| 667 | A | A |
| 668 | A | A |
| 669 | A | A |
| 670 | A | A |
| 671 | A | A |
| 672 | A | A |
| 673 | A | A |
| 674 | A | A |
| 675 | A | A |
| 676 | A | A |
| 677 | A | A |
| 678 | A | A |
| 679 | A | A |
| 680 | A | A |
| 681 | A | A |
| 682 | A | A |
| 683 | A | A |
| 684 | A | A |
| 685 | A | A |
| 686 | A | A |
| 687 | A | A |
| 688 | A | A |
| 689 | A | A |
| 690 | A | A |
| 691 | A | A |
| 692 | A | A |
| 693 | A | A |
| 694 | A | A |
| 695 | A | A |
| 696 | A | A |
| 697 | A | A |
| 698 | A | A |
| 699 | A | A |
| 700 | A | A |
| 701 | A | A |
| 702 | A | A |
| 703 | A | A |
| 704 | A | A |
| 705 | A | A |
| 706 | A | A |
| 707 | A | A |
| 708 | A | A |
| 709 | A | A |
| 710 | A | A |
| 711 | A | A |
| 712 | A | A |
| 713 | A | A |
| 714 | A | A |
| 715 | A | A |
| 716 | A | A |
| 717 | A | A |
| 718 | A | A |
| 719 | A | A |
| 720 | A | A |
| 721 | A | A |
| 722 | A | A |
| 723 | A | A |
| 724 | A | A |
| 725 | A | A |
| 726 | A | A |
| 727 | A | A |
| 728 | B | B |
| 729 | A | A |
| 730 | A | A |
| 731 | A | B |
| 732 | A | A |
| 733 | B | D |
| 734 | B | D |
| 735 | A | B |
| 736 | D | D |
| 737 | A | A |
| 738 | A | A |
| 739 | A | A |
| 740 | A | A |
| 741 | — | — |

### Example B-2: CellTiter-Glo Luminescent Cell Viability Assay

Cell Titer Glo Viability Assay with HT-29 Cells (72h Incubation)

The CellTiter-Glo Luminescent Cell Viability Assay (Promega) is a homogeneous method of determining the number of viable cells in culture. It is based on quantification of ATP, indicating the presence of metabolically active cells.

On day 1 400 HT-29 cells per well are seeded in white 384well plates (Greiner, #781080) in 25 μl cell culture medium (DMEM (PAN-Biotech #P04-03590)+10% FCS+glutamine). After incubation for 24 h at 37° C./5% CO₂ compounds or DMSO are added at different concentrations by Echo Liquid Handling Technology. Cells are further incubated in humidified chambers for 72 h at 37° C. and 5% CO₂. Cells treated with the compound vehicle DMSO are used as positive controls and cells treated with 10 μM Staurosporine serve as negative controls.

At day 5-72h after compound addition—the CellTiter Glo Reagent is prepared according to the instructions of the kit (Promega Inc.): Reagent is mixed 1:1 with cell culture medium. Thereon, mixture and assay plates are equilibrated at room temperature for 20 min. Equal volumes of the reagent-medium-mixture is added to the volume of culture medium present in each well. The plates are mixed at −300 rpm for 2 minutes on an orbital shaker. The microplates are then incubated at room temperature for 10 minutes for stabilization of the luminescent signal. Following incubation the luminescence is recorded on a Victor microplate reader (Perkin Elmer) using a 200 ms integration time. The data is then analyzed with Excel using the XLFIT Plugin (dose response Fit 205) for IC₅₀-determination.

As quality control the Z′-factor is calculated from 16 positive and negative control values. Only assay results showing a Z′-factor≥0.5 are used for further analysis.

Table 3 shows activity data in the biochemical Glo cell viability assays of A549 (human lung carcinoma cell line) and Karpas and CTG assay of HT29 (human colon cancer cell line) cell. Inhibition is indicated as IC₅₀ [nM] (“-”=not measured). Compounds having an activity designated as “A” provided an IC₅₀≤100 nM; compounds having an activity designated as “B” provided an 100 nM<IC₅₀≤500 nM; compounds having an activity designated as “C” provided an 500 nM<IC₅₀≤1000 nM; compounds having an activity designated as “D” provided an 1000 nM<IC₅₀≤5000 nM; compounds having an activity designated as “E” provided an IC₅₀>5000 nM.

TABLE 3

| Cpd | PGLo A549 | Pglo Karpas | CTG HT29 |
|---|---|---|---|
| No. | [nM] | [nM] | [nM] |
| 7 | A | | A |
| 8 | C | A | B |
| 13 | C | | C |
| 19 | D | C | D |
| 16 | C | | B |
| 22 | — | | D |
| 25 | A | | A |
| 26 | A | | A |
| 27 | A | | A |
| 28 | A | A | A |
| 29 | A | | A |
| 30 | A | | A |
| 31 | A | | A |
| 32 | A | | A |
| 33 | A | | A |
| 34 | A | | A |
| 35 | B | | B |
| 36 | B | | B |
| 37 | A | | A |
| 38 | | | A |
| 39 | A | | A |
| 40 | A | | A |
| 41 | | | A |
| 42 | A | | A |
| 43 | B | | A |
| 44 | | | A |
| 45 | B | | B |
| 51 | B | | D |
| 59 | ND | | D |
| 60 | B | B | A |
| 61 | | | A |
| 62 | | | A |
| 63 | A | A | A |
| 64 | A | A | A |
| 77 | C | B | D |
| 78 | E | B | |
| 79 | D | C | D |
| 80 | A | A | A |
| 81 | A | A | A |
| 82 | A | A | A |
| 83 | A | A | A |
| 84 | A | A | A |
| 85 | A | A | A |
| 86 | A | A | A |
| 87 | A | A | A |
| 88 | A | A | A |
| 89 | A | A | A |
| 95 | D | | C |
| 107 | — | | A |
| 116 | E | | B |
| 120 | D | | B |
| 126 | A | | B |
| 127 | A | | A |
| 128 | A | | A |
| 129 | A | | A |
| 130 | B | | B |
| 134 | A | | A |
| 135 | A | | A |
| 136 | A | | B |
| 143 | B | | B |
| 148 | E | | B |
| 151 | | | A |
| 152 | A | | A |
| 160 | B | | A |
| 157 | B | | A |
| 158 | A | | A |
| 159 | B | | A |
| 168 | — | | B |
| 171 | C | | B |
| 172 | — | | A |
| 173 | — | | B |
| 174 | A | | A |
| 175 | A | | A |
| 176 | A | | A |
| 177 | A | | A |
| 179 | A | | A |
| 180 | A | | A |
| 181 | A | | A |
| 182 | A | | A |
| 186 | B | | B |
| 191 | D | | D |
| 195 | B | | A |
| 199 | A | | A |
| 205 | A | | A |
| 211 | C | | B |
| 213 | B | | A |
| 215 | B | | A |
| 216 | B | | A |
| 217 | A | | A |
| 218 | | | A |
| 219 | A | | A |
| 220 | A | | A |
| 252 | | | B |
| 253 | | | B |
| 254 | | | B |
| 255 | E | | B |
| 256 | | | B |
| 257 | | | B |
| 258 | C | | B |
| 259 | | | B |
| 260 | | | B |
| 261 | | | B |
| 265 | E | | B |
| 269 | D | | D |
| 281 | D | | B |
| 284 | B | | A |
| 285 | B | | A |
| 286 | B | | A |
| 287 | A | | A |
| 288 | B | | A |
| 289 | C | | B |
| 290 | E | | B |
| 291 | D | | A |
| 293 | A | | A |
| 294 | A | | A |
| 292 | A | | A |
| 282 | E | | B |
| 295 | B | | A |
| 296 | C | | B |
| 298 | | | B |
| 299 | E | | B |
| 300 | D | | B |
| 301 | D | | B |
| 283 | D | | B |
| 302 | A | | A |
| 303 | | | A |
| 304 | | | A |
| 305 | | | A |
| 306 | | | A |
| 307 | | | A |
| 308 | | | A |
| 309 | A | | A |
| 310 | A | | A |
| 311 | A | | A |
| 312 | | | B |
| 313 | | | B |
| 314 | A | | A |
| 315 | | | A |
| 316 | | | A |
| 317 | | | A |
| 318 | | | A |
| 319 | | | A |
| 320 | A | | A |
| 322 | D | | B |
| 323 | A | | A |
| 324 | A | | A |
| 325 | A | | A |
| 326 | A | | A |
| 327 | B | | A |
| 328 | B | | A |
| 329 | B | | A |
| 330 | A | | A |
| 331 | A | | A |
| 332 | A | | A |
| 333 | A | | A |
| 334 | B | | A |
| 335 | A | | A |
| 336 | A | | A |
| 337 | B | | A |
| 338 | B | | A |
| 339 | B | | A |
| 340 | A | | B |
| 341 | A | | A |
| 342 | A | | A |
| 343 | A | | A |
| 344 | A | | A |
| 345 | B | | A |
| 347 | B | | A |
| 348 | A | | A |
| 349 | B | | B |
| 350 | A | | A |
| 351 | B | | B |
| 352 | A | | A |
| 353 | A | | A |
| 354 | | | B |
| 355 | E | | D |
| 356 | C | | C |
| 358 | A | | B |
| 359 | A | | A |
| 360 | A | | A |
| 361 | B | | A |
| 362 | A | | A |
| 363 | A | | A |
| 364 | A | | A |
| 369 | A | | A |
| 371 | A | | A |
| 372 | A | | A |
| 373 | A | A | |
| 495 | | | B |
| 503 | B | | A |
| 571 | A | | C |
| 505 | A | | A |
| 572 | | | B |
| 509 | A | | B |
| 510 | A | | A |
| 511 | A | | A |
| 512 | A | | A |
| 513 | A | | A |
| 549 | B | | A |
| 550 | | | A |
| 551 | A | | A |
| 552 | B | | A |
| 553 | B | | A |
| 554 | B | | A |
| 555 | A | | A |
| 556 | A | | A |
| 557 | A | | B |
| 558 | B | | A |
| 559 | B | | A |
| 560 | B | | A |
| 561 | A | | A |
| 562 | B | | B |
| 563 | A | | A |
| 564 | A | | A |
| 565 | A | | A |
| 566 | A | | B |
| 567 | A | | B |
| 568 | A | | B |
| 573 | A | | |
| 574 | A | | |
| 575 | A | | |
| 576 | A | | |
| 577 | A | | |
| 578 | A | | |
| 579 | A | | |
| 580 | A | | |
| 581 | A | | |
| 582 | A | | |
| 583 | A | | |
| 584 | A | | |
| 585 | A | | |
| 586 | A | | |
| 587 | A | | |
| 588 | A | | |
| 589 | A | | |
| 590 | A | | |
| 591 | A | | |
| 592 | A | | |
| 593 | A | | |
| 594 | A | | |
| 595 | A | | |
| 596 | A | | |
| 597 | A | | |
| 598 | A | | |
| 599 | A | | |
| 600 | — | | |
| 601 | A | | |
| 602 | A | | |
| 603 | A | | |
| 604 | A | | |
| 605 | A | | |
| 606 | A | | |
| 607 | A | | |
| 608 | A | | |
| 609 | A | | |
| 610 | A | | |
| 611 | A | | |
| 612 | A | | |
| 613 | A | | |
| 614 | A | | |
| 615 | A | | |
| 616 | A | | |
| 617 | A | | |
| 618 | A | | |
| 619 | A | | |
| 620 | A | | |
| 621 | A | | |
| 622 | A | | |
| 623 | A | | |
| 624 | A | | |
| 625 | A | | |
| 626 | A | | |
| 627 | A | | |
| 628 | A | | |
| 629 | A | | |
| 630 | A | | |
| 631 | A | | |
| 632 | A | | |
| 633 | A | | |
| 634 | A | | |
| 635 | A | | |
| 636 | A | | |
| 637 | A | | |
| 638 | A | | |
| 639 | A | | |
| 640 | A | | |
| 641 | A | | |
| 642 | A | | |
| 643 | A | | |
| 644 | A | | |
| 645 | A | | |
| 646 | A | | |
| 647 | A | | |
| 648 | A | | |
| 649 | A | | |
| 650 | A | | |
| 651 | A | | |
| 652 | A | | |
| 653 | A | | |
| 654 | A | | |
| 655 | A | | |
| 656 | A | | |
| 657 | A | | |
| 658 | A | | |
| 659 | A | | |
| 660 | A | | |
| 661 | A | | |
| 662 | A | | |
| 663 | A | | |
| 664 | A | | |
| 665 | A | | |
| 666 | A | | |
| 667 | A | | |
| 668 | A | | |
| 669 | A | | |
| 670 | A | | |
| 671 | A | | |
| 672 | A | | |
| 673 | A | | |
| 674 | A | | |
| 675 | A | | |
| 676 | A | | |
| 677 | B | | |
| 678 | A | | |
| 679 | A | | |
| 680 | A | | |
| 681 | A | | |
| 682 | A | | |
| 683 | A | | |
| 684 | A | | |
| 685 | A | | |
| 686 | A | | |
| 687 | A | | |
| 688 | A | | |
| 689 | A | | |
| 690 | A | | |
| 691 | A | | |
| 692 | A | | |
| 693 | A | | |
| 694 | A | | |
| 695 | A | | |
| 696 | A | | |
| 697 | A | | |
| 698 | A | | |
| 699 | A | | |
| 700 | A | | |
| 701 | A | | |
| 702 | A | | |
| 703 | A | | |
| 704 | A | | |
| 705 | A | | |
| 706 | A | | |
| 707 | A | | |
| 708 | A | | |
| 709 | A | | |
| 710 | A | | |
| 711 | B | | |
| 712 | A | | |
| 713 | A | | |
| 714 | A | | |
| 715 | A | | |
| 716 | A | | |
| 717 | A | | |
| 718 | A | | |
| 719 | A | | |
| 720 | A | | |
| 721 | B | | |
| 722 | B | | |
| 723 | A | | |
| 724 | A | | |
| 725 | A | | |
| 726 | B | | |
| 727 | B | | |
| 728 | C | | |
| 729 | A | | |
| 730 | B | | |
| 731 | D | | |
| 732 | D | | |
| 733 | D | | |
| 734 | D | | |
| 735 | E | | |
| 736 | E | | |
| 737 | A | | |
| 738 | A | | |
| 739 | A | | |
| 740 | B | | |
| 741 | — | | |

### Example B-3: Chymotrypsin-Like ProteasomeGlo Assay

Chymotrypsin-like ProteasomeGlo assay in the B-cells isolated from PBMC's following compound addition (IC₅₀).
1. PBMC's will be isolated from human blood followed by B-cell isolation from the PBMC's using the Miltenyi Isolation Kit.
2. This experiment will use the Chymotrypsin-like ProteasomeGlo read-out to generate IC₅₀'s following compound treatment.
3. Dilute all compounds 1:100 for the assay.
4. Do not make a staurosporine transfer for lane 12! Repeat the DMSO transfer with lane 12.
5. Lane 12 should not be seeded with any cells. Only medium!
6. For all steps, make sure the samples and reagents remain cool!
7. While dealing with the buffy coats, be careful to not let any exposed skin to be in direct contact with the blood.

### Reagents

### Separating Medium

Lymphocyte Separating Medium (Pancoll) (PAN Biotech; P04-60125)
RBC Lysis Buffer (10×) (BioLegend; 420301)
Prepare 1×RBC Lysis Buffer by diluting 10×RBC Lysis Buffer to 1× with ddH₂O.

### PBMC Cell Medium

| | | | | Final |
|---|---|---|---|---|
| | Manufacturer | Concen- | Volume | concen- |
| Component | information | tration | (ml) | tration |
| RPMI 1640 | PAN Biotech; P04-22100 | | 500 | |
| HyClone Heat | Thermo Scientific; | 100% | 50 | 10% |
| inactivated | SV30180.03 (Lot No.: | | | |
| FCS | TXK40002) | | | |
| L-Glutamine | PAN Biotech; P04-80100 | 100x | 5 | 1X |

### Cultivation Medium for B-Cells

| | | | | Final |
|---|---|---|---|---|
| | Manufacturer | Concen- | Volume | concen- |
| Component | information | tration | (ml) | tration |
| DMEM | Gibco; 41965039 | | 500 | |
| Heat inactivated | Capricon; FBS-11A | 100% | 50 | 10% |
| FCS | | | | |
| L-Glutamine | PAN Biotech; P04-80100 | 100x | 5 | 1X |

### Wash Buffer

| | | | | Final |
|---|---|---|---|---|
| | Manufacturer | Concen- | Volume | concen- |
| Component | information | tration | (ml) | tration |
| DPBS | Gibco; 14190136 | | 178 | 89% |
| DMEM | Gibco; 41965039 | | 20 | 10% |
| Heat inactivated | Capricon; FBS-11A | 100% | 2 | 1% |
| FCS | | | | |

### B Cell Isolation Buffer

| | | | Final | | |
|---|---|---|---|---|---|
| | Manufacturer | Concen- | concen- | | |
| Component | information | tration | tration | Amount | Unit |
| DPBS | Gibco; | | | 300 | ml |
| | 14190136 | | | | |
| Bovine Serum | | | 0.50% | 1.5 | g |
| albumin (BSA) | | | | | |
| EDTA Solution | Self-made | 250 mM | 2 mM | 2.4 | ml |

Chymotrypsin-Like Cell-Based Proteasome-Glo™ Assay (Promega; G8661)
Cell culture 384-well plates (Greiner BioOne; 781080)
Dispensing cassette, MultiDrop Standard tube dispensing cassette (Thermo Scientific; 24072670)
DPBS (Pan BioTech; P04-36500)
Cell culture 384-well plates (Greiner BioOne; 781080)
CPD source plate: diamond-shape plate (Labcyte; LP-0200)
Intermediate plate (Labcyte; P-05525)

| | |
|---|---|
| Day 1 | Seed A549 cells |
| Day 2 | Seed Karpas cells and ProteasomeGlo assay |

### B Cell Isolation Kit II, Human (Miltenyi Biotec; 130-091-151)

includes,

### 1. QuadroMACS™ Starting Kit (LS) (#130-091-051)

QuadroMACS™ Separator ((#1 30-090-976)
MACS MultiStand ((#130-042-303)
LS Columns (#130-042-401)
MACS® 15 mL Tube Rack (#130-091-052)
One unit of MACS Microbeads, or one MACS
Microbead Kit, or one MACS Cell Isolation Kit.
   2. B cell Isolation Kit II

| | |
|---|---|
| Components: | 1 mLB Cell Biotin-Antibody Cocktail, human: Cocktail of |
| | biotin-conjugated monoclonal antibodies against CD2, |
| | CD14, CD16, CD36, Cd43, and CD235a (Glycophorin A)- |
| | 2 mL Anti-Biotin Microbeads: Microbeas conjugated to |
| | monoclonla anti-biotin antibodies (isotype: mouse IgG1). |
| Capacity | For 10⁹ total cells |
| Product | All components are supplied in buffer containing stabilizer |
| format | and 0.05% sodium azide. |
| Storage | Store protected from light at 2-8° C. Do not freeze. The |
| | expiration date is indicated on the vial labels. |

### Components of the Miltenyi Biotec Starting and B Cell Isolation Kit

MACS® MultiStand
LS Columns
QuadroMACS™ Separator

### Method

Day 1: PBMC isolation followed by B-cell isolation using the Miltenyi Kit
Day 2: B-cell plating, ECHO transfer and ProteasomeGlo assay

### Day 1

### 1. Prepare the Wash Buffer and Cultivation Medium for the Assay.

a. Cultivation medium: Prepare and store at 4° C. The cultivation medium will be used at the end of B-cell isolation for overnight incubation of the B-cells and subsequent experiment.
b. Wash buffer: Prepare fresh before each assay taking into care to be exact with the volumes prepared.
   ˜200 ml of the wash buffer is required per B-cell isolation from PBMCs.
c. Buffer for B cell isolation: Prepare the buffer fresh before each assay as it contains BSA which is unstable in a solution for long term storage. Cool the buffer own on ice before use!
d. PBMC cell medium: Prepare and store at 4° C. The medium is to be pre-warmed before starting the PBMC isolation from the buffy coats.

### 2. Assemble the Components of the Miltenyi Separator+MACS MultiStand as Follows:

a. Attach the QuadroMACS Separator to the MACS MultiStand by placing the separator adjacent to the MultiStand and slowly sliding the separator onto the stand. not to trap your fingers while attaching the two components together.
b. Check that the ejection blocks in the gap pf the magnet (separator) are attached before placing the MACS LS columns into the magnetic field of the separator.
c. Place the LS Columns with the column wings to the front into the slots of the separator.
   3. PBMC Isolation from Buffy Coats

For the initial steps and to make processing the buffy coats easier, separately process the buffy coats.
a. Cut the tube attached to the buffy coats carefully using a clean, sterile and fresh scalpel.
b. Transfer the blood into 2 individual 50 ml falcons.
c. Add an equal volume of DPBS to the blood in the falcons.
d. Add ˜20 ml of lymphocyte separating medium to two 50 ml falcons.
e. Carefully pour 15-20 ml of the blood+DPBS mix from step c. onto the lymphocyte separating medium from step d.
f. Centrifuge the falcons with the lymphocyte separating medium+blood mix at 800×g for 15 minutes using a swing-out rotor.
g. Following centrifugation, carefully remove the falcons from the centrifuge.
h. The following layers will be observed:
i. Aspirate the plasma layer off carefully without disturbing the different layers.
j. Remove the cell pellet using a pipette into two fresh 50 ml falcons.
k. Fill the falcons with the cells up to 50 ml with DPBS.
l. Centrifuge the falcons at 250×g for 10 minutes using a swing-out rotor (Brake-off).
m. Aspirate the complete DPBS off the cells without disturbing the cell pellet at the bottom of the falcon.
n. Re-suspend the cell pellet in 10 ml of 1×RBC lysis buffer.
o. Incubate the falcons at room temperature in the dark for 10 minutes.
p. After RBC lysis, centrifuge the tubes at 250×g for 10 minutes using a swing-out rotor (brake-off).
(*Note: If the cell pellet is still red or light red, repeat steps n. to p.!)
q. Re-suspend the cell pellet in DPBS up to 50 ml in the falcon.
r. Centrifuge the falcons at 250×g for 10 minutes using a swing-out rotor (Brake-off).
s. Aspirate the DPBS off the cells without disturbing the cell pellet at the bottom of the falcon.
t. Repeat the wash step as described in steps q. to s. 2× times more (total number of washes is 3×).
u. Aspirate the DPBS completely off the cell pellet after the last wash without disturbing the cell pellet.
v. Re-suspend the cell pellet in 20 ml of PBMC cell medium.
w. Proceed to counting of cells and B-cell isolation from PBMCs.
(*Note: Keep the cells isolated from the two donors or more always separate and do not pool the cells from multiple donors at any step!(
   4. B Cell Isolation from PBMCs
a. Collect the PBMCs and re-suspend the entire amount of cells into 30 ml of wash buffer in a 50 ml falcon.
b. Count the PBMCs using the CEDEX.

| | Patient 1 (719) | Patient 2 (724) |
|---|---|---|
| Number of PBMC/ml | 17774000 | 13101000 |
| Total volume of suspension | 40 ml | 40 ml |
| Total number of PBMCs | 710960000 | 524040000 |

c. Leave the cell suspension at room temperature for 30 mins.
d. Centrifuge the cell suspension at the end of 30 minutes for 10 mins at 1500 rpm.
e. Aspirate the wash buffer carefully from the cells without disturbing the pellet at the bottom of the falcon.
f. Re-suspend the pellet in volume of the B-cell isolation buffer calculated as follows:

| | | | | |
|---|---|---|---|---|
| Volume of B-cell isolation buffer | 40 | μl | 40 | μl |
| per 10⁷ total cells | | | | |

| | | |
|---|---|---|
| Total number of cells | 710960000 | 524040000 |

| | | | | |
|---|---|---|---|---|
| Total volume of B-cell isolation | 2843.84 | μl | 2096.16 | μl |
| buffer needed for re-suspension | | | | |

g. Add the calculated volume of Biotin-Antibody Cocktail as follows:

| Volume of Biotin-Antibody cocktail | 10 | μl | 10 | μl |
|---|---|---|---|---|
| per 10⁷ total cells | | | | |
| Total number of cells | 710960000 | | 524040000 | |
| Total volume of Biotin-Antibody | 710.96 | μl | 524.04 | μl |
| cocktail to be added | | | | |

h. Mix well using a 1 ml pipette and incubate on ice for 10 mins.
i. Add more B-cell isolation buffer to the cell+antibody suspension as follows:

| | | | | |
|---|---|---|---|---|
| Volume of B-cell isolation buffer | 30 | μl | 30 | μl |
| per 10⁷ total cells | | | | |

| | | |
|---|---|---|
| Total number of cells | 710960000 | 524040000 |

| | | | | |
|---|---|---|---|---|
| Total volume of B-cell isolation | 2132.88 | μl | 1572.12 | μl |
| buffer to be added | | | | |

j. Add the calculated volume of anti-Biotin Microbeads to the cell+antibody mix as follows:

| | | | | |
|---|---|---|---|---|
| Volume of anti-Biotin microbeads | 20 | μl | 20 | μl |
| per 10⁷ total cells | | | | |

| | | |
|---|---|---|
| Total number of cells | 710960000 | 524040000 |

| | | | | |
|---|---|---|---|---|
| Total volume of Biotin-Antibody | 1421.92 | μl | 1048.08 | μl |
| cocktail to be added | | | | |

k. Mix well using a 1 ml pipette and incubate the mix on ice for 20 mins.
l. In the meantime, equilibrate one LS Column as follows:
   1. Insert one LS Column into one slot of the separator as depicted in the pictures above.
   2. Place a 50 ml falcon at the bottom of the LS column to collect the wash flow-through.
   3. Add 3 ml of the B-cell isolation buffer into the column.
   4. Allow the buffer to flow-through making sure that the entire volume passes the column and that the column reservoir is empty before the addition of the cells.
   5. Remove the falcon with the flow-through and discard.
m. Place a fresh 50 ml falcon under the LS Column to collect the untouched B-cells isolated from the PBMCs.
n. Apply the entire volume of the cell+antibody+microbeads mix to the LS Column.
o. Collect the flow-through from each application of the mix into the same 50 ml falcon under the LS Column.
p. Once the complete volume of the cell+antibody+microbead mix has been passed through, proceed to washing of the column.
q. Add 3 ml of the B-cell isolation buffer to the LS Column and allow the buffer to flow-through. Collect the flow-through and repeat the wash 2× times more (in total 3× washes).
r. Centrifuge the B-cells isolated in the collection falkan at 1500 rpm and 4° C. for 15 mins.
s. Aspirate the B-cell isolation buffer off the pellet keeping in mind the relatively low attachment of the B-cells to tubes and that the B-cells are not aspirated off.
t. Re-suspend the B-cell pellet in 1000 μl of fresh B-cell isolation buffer.
u. Collect 50 μl of the B-cell suspension into a fresh, labelled eppi and add 25 μl of 3× Laemmli buffer to the cell suspension (Sample)
v. Make up the volume of the B-cell suspension to 10 ml with B-cell cultivation medium and store the suspension overnight at 4° C.
w. Place a fresh 2 ml eppendorf under the LS Column. Remove the column from the separator and carefully depress the plunger through the column, collecting the unwanted cells removed from the B-cells (Pass).
x. Add 100 μl of 3× Laemmli buffer to the Pass from step u and 25 μl of 3× Laemmli buffer to the Input collected in step f.
y. Boil all three samples (Input, Sample and Pass) at 98° C. for 15 mins.
z. Proceed to western blotting from the samples prepared.
a1. For the B-cell suspension stored overnight, proceed to B-cell counting and plating for cell number titration on Day 2.

### Day 2

Proceed directly with the cell suspension from Day 1 for cell counting.
1. Count the cells using the CEDEX

| | | |
|---|---|---|
| Cell number required per well | 10.000 | /25 μl |
| Number of cells/ml | 400.000 | |
| Number of wells | 384 | |
| Total number of plates | 3 | |
| Volume of cell suspension to be seeded per well | 25 | μl |
| Total volume of cell suspension required | 28800 | μl |
| Total Volume of cells required | 20000000 | |

| | B-cells | B-cells | |
|---|---|---|---|
| Number of cell/ml | 21777000 | 14331000 | |
| Number of cells/ml | | | |
| Total volume of cells suspension | 0.91840015 ml | 1.39557602 | ml |
| Volume of medium required | 49.0815999 ml | 48.604424 | ml |

2. Using a multi-channel pipette, pipette 25 μl of the cell suspensions
3. Place the cells for 3 hrs in the high humidity incubator at 37° C. and 5% CO₂.

### ECHO Compound Transfer

Destination plate refers to the plates onto which the compounds will be transferred (meaning the plates with the cells already plated).

1. Compound Plate from Compound Management:
a. Pipette 2×8 μl of the compounds (10 mM or 1 mM stock solutions) into rows A1-P1, A₂-P2 and A3-L3 of a diamond shaped plate.
b. Once pipetted, seal the compound plate with an aluminum foil seal and spin down briefly. Place the plate in the dark till further use.

### 2. DMSO Intermediate Plate:

a. Pipette 25 μl of DMSO into columns 1-12 of an intermediate plate.
b. Seal the lanes 1-12 using an aluminum foil seal.
c. Spin the plate down briefly and store till further use.

### 4. ECHO Transfer

a. Remove the plates with cells from the incubator.
b. Take all the plates including the compound plate, intermediate plate and Staurosporine plate to the ECHO.
c. Click on “Open” in the ECHO software and select the protocol “QLI5_25ul_30 uM-Grenier”.
d. Follow the on-screen prompts and instructions once you click “Start” to insert the compound plate, intermediate plate and destination plates.
e. Change the final number of destination plates on the program.
f. Follow the on-screen prompts and instructions once you click “Start” to load the Staurosporine and destination plates.
g. Once all the compounds, DMSO and Staurosporine have been transferred, place the destination plates in a wet tissue box.
h. Place the destination plates back into the incubator and incubate at 37° C., 5% CO₂ for 105 minutes.
i. Re-seal all the compound+Stauro diamond and intermediate plates and store in the dark.
   Chymotrypsin like ProteasomeGlo Assay
1. Thaw the components of the ProteasomeGlo assay at room temperature including the ProteasomeGlo Buffer, the lyophilized Luciferin Detection Reagent and the Suc-LLVY-Glo Substrate.
2. Reconstitute the Luciferin Detection Reagent in the amber coloured bottle by adding 50 ml of the ProteasomeGlo Buffer.
3. Vortex the Suc-LLVY-Glo Substrate briefly to reconstitute the Substrate well.
4. Spin down the Substrate and add 250 μl of the Substrate to the reconstituted Luciferin Detection Reagent from step 2.
5. Briefly vortex the amber bottle with the mix to prepare a homogenous solution.
6. Place the mix prepared in step 5 to sit at room temperature for 60 minutes.
7. After 60 minutes, pipette 25 μl of the mix from step 5 into all wells with the cells.
8. Shake the plates for 2-5 minutes to ensure the complete mixing of the ProteasomeGlo reagent with the cells.
9. Leave the plates in the dark for 10-15 minutes.
10. Measure the luminescence read-out using the Viktor and the protocol “Luminescence_384Well_0.2 sec”.
   (Note: The Protoeasome-Glo™ Reagent (combined Proteasome-Glo™ Substrate, Proteasome-Glo™ Buffer and Luciferin Detection Reagent) can be stored at 4° C. or −20° C. for 1 month with minimal loss of activity.)

Table 4 shows activity data in the Chymotrypsin-like ProteasomeGlo assay of B-cell and hPMBC. Inhibition is indicated as IC₅₀ [nM] (“-”=not measured). Compounds having an activity designated as “A” provided an IC₅₀≤100 nM; compounds having an activity designated as “B” provided an 100 nM<IC₅₀≤500 nM; compounds having an activity designated as “C” provided an 500 nM<IC₅₀≤1000 nM; compounds having an activity designated as “D” provided an 1000 nM<IC₅₀≤5000 nM; compounds having an activity designated as “E” provided an IC₅₀>5000 nM.

TABLE 4

| Cpd | Pglo B-Cell | Pglo hPBMC | B cell diff. | PBMC IL 17 stim. |
|---|---|---|---|---|
| No. | [nM] | [nM] | [nM] | [nM] |
| 7 | | A | | |
| 8 | A | B | | |
| 13 | A | | B | |
| 16 | A | | A | A |
| 22 | | | | |
| 25 | A | | | |
| 26 | A | | | |
| 27 | A | | A | A |
| 28 | | B | | |
| 29 | A | | A | A |
| 30 | A | | A | |
| 31 | A | | | |
| 32 | A | | | |
| 33 | A | | A | |
| 34 | A | | A | |
| 35 | A | | A | |
| 36 | B | | A | |
| 37 | A | | A | |
| 39 | A | | | |
| 40 | A | | | |
| 42 | A | | A | |
| 43 | A | | A | |
| 44 | | | | |
| 45 | B | | A | |
| 51 | A | | | |
| 59 | B | | | |
| 60 | B | B | | |
| 63 | A | A | | A |
| 64 | A | A | | |
| 77 | B | B | | |
| 78 | B | C | | |
| 79 | C | | | |
| 80 | A | A | | |
| 81 | A | A | | |
| 82 | A | A | | |
| 83 | A | A | | |
| 84 | | A | | |
| 85 | | A | | |
| 86 | | A | | |
| 87 | | A | | |
| 88 | A | A | | |
| 89 | | A | | |
| 116 | | C | | |
| 120 | | B | | |
| 126 | | A | | |
| 127 | | A | | |
| 128 | | A | | |
| 129 | | A | | |
| 130 | | A | | |
| 134 | | A | | |
| 135 | | A | | |
| 136 | | A | | |
| 143 | | A | | |
| 148 | | C | | |
| 151 | | | | |
| 152 | | A | | |
| 160 | | A | | |
| 157 | | A | | |
| 158 | | A | | |
| 159 | | A | | |
| 168 | | D | | |
| 171 | | A | | |
| 174 | | A | | |
| 175 | | A | | |
| 176 | | A | | |
| 177 | | A | | |
| 179 | | A | | |
| 180 | | A | | |
| 181 | | A | | |
| 182 | | A | | |
| 186 | | B | | |
| 191 | | B | | |
| 195 | | A | | |
| 199 | | A | | |
| 205 | | A | | |
| 211 | | B | | |
| 213 | | A | | |
| 215 | | A | | |
| 216 | | A | | |
| 217 | | A | | |
| 219 | | A | | |
| 220 | | A | | |
| 253 | | E | | |
| 255 | | E | | |
| 256 | | E | | |
| 257 | | D | | |
| 258 | | B | | |
| 259 | | E | | |
| 260 | | E | | |
| 281 | | D | | |
| 284 | | A | | |
| 285 | | A | | |
| 286 | | A | | |
| 287 | | A | | |
| 288 | | A | | |
| 289 | | C | | |
| 290 | | E | | |
| 291 | | D | | |
| 293 | | A | | |
| 294 | | A | | |
| 292 | | A | | |
| 282 | | C | | |
| 295 | | B | | |
| 296 | | B | | |
| 299 | | D | | |
| 300 | | C | | |
| 301 | | D | | |
| 283 | | C | | |
| 302 | | A | | |
| 309 | | A | | |
| 310 | | A | | |
| 311 | | A | | |
| 314 | | A | | |
| 320 | | A | | |
| 322 | | C | | |
| 326 | | A | | |
| 329 | | B | | |
| 332 | | A | | |
| 333 | | A | | |
| 334 | | B | | |
| 335 | | A | | |
| 336 | | A | | |
| 337 | | A | | |
| 338 | | A | | |
| 339 | | B | | |
| 340 | | A | | |
| 341 | | A | | |
| 342 | | A | | |
| 343 | | A | | |
| 344 | | A | | |
| 345 | | A | | |
| 347 | | A | | |
| 348 | | A | | |
| 349 | | A | | |
| 350 | | A | | |
| 351 | | A | | |
| 352 | | B | | |
| 353 | | A | | |
| 354 | | A | | |
| 355 | | A | | |
| 356 | | A | | |
| 358 | | A | | |
| 359 | | A | | |
| 360 | | A | | |
| 361 | | A | | |
| 362 | | A | | |
| 363 | | A | | |
| 364 | | A | | |
| 371 | | A | | |
| 372 | | A | | |
| 373 | A | A | | |
| 503 | | B | | |
| 505 | | B | | |
| 509 | | B | | |
| 510 | | A | | |
| 511 | | A | | |
| 512 | | A | | |
| 513 | | A | | |
| 549 | | A | | |
| 550 | | | | |
| 551 | | A | | |
| 552 | | A | | |
| 553 | | A | | |
| 554 | | B | | |
| 555 | | A | | |
| 556 | | A | | |
| 557 | | A | | |
| 558 | | A | | |
| 559 | | A | | |
| 560 | | A | | |
| 561 | | A | | |
| 562 | | A | | |
| 563 | | A | | |
| 564 | | A | | |
| 565 | | B | | |
| 566 | | B | | |
| 567 | | A | | |
| 568 | | A | | |
| 571 | | A | | |

### Example B-4: CTG Assays

A viability assay was conducted for 48 hours to evaluate the inhibition of cancer cell growth by proteasome inhibitors. Briefly, the candidate cell line was plated with a 96-well plate following density of cells, respectively. 6×10³ Cells/well for A2780 (human ovarian cancer cell line) and MDA-MB-468 (breast carcinoma), 8×10³ Cells/well for Hs746T (human gastric carcinoma cell line), 1.1×10⁴ Cells/well for MM1 S and RPMI 8226 (multiple myeloma). After 24 hours, the cells were treated with various concentrations of the compound (ranging from 0.0015 uM to 10 uM). DMSO solvent without compound served as control and the final DMSO concentration was less than 0.1%. After 48 hours of incubation at 37° C., 5% CO₂ incubator, cells were analyzed for viability using the CellTiter-Glo Luminescent Cell Viability assay (Promega, Cat #G7570). All viability assays were performed in duplicate and Luminescence was read using an Envision (Perkin Elmer, USA). Data were analyzed using XLfit software.

Table 5 shows activity data in the 48 hr CTG assays of A2780 (human ovarian cancer cell line), MDA-MB-468 (breast carcinoma), Hs 746T (human gastric carcinoma cell line), MM1 S (B lymphoblast cell line), and RPMI 8226 (B lymphocyte cell line, multiple myeloma). Inhibition is indicated as IC₅₀ [nM] (“-”=not measured). Compounds having an activity designated as “A” provided an IC₅₀≤100 nM; compounds having an activity designated as “B” provided an 100 nM<IC₅₀≤500 nM; compounds having an activity designated as “C” provided an 500 nM<IC₅₀≤1000 nM; compounds having an activity designated as “D” provided an 1000 nM<IC₅₀≤5000 nM; compounds having an activity designated as “E” provided an IC₅₀>5000 nM.

TABLE 5

| | A2780 | MDA-MB-468 | Hs 746T | MM1S | RPMI-8226 |
|---|---|---|---|---|---|
| Cpd | IC₅₀ [nM] | IC₅₀ [nM] | IC₅₀ [nM] | IC₅₀ [nM] | IC₅₀ [nM] |
| 573 | | | | A | A |
| 574 | | A | A | A | A |
| 575 | | | A | A | A |
| 576 | | | A | A | A |
| 577 | | | A | A | A |
| 578 | | | A | A | A |
| 579 | | | A | A | A |
| 580 | | | A | A | A |
| 581 | | | A | A | A |
| 582 | | | A | A | A |
| 583 | | | A | A | A |
| 584 | | | A | A | A |
| 585 | | | A | A | A |
| 586 | | | A | A | A |
| 587 | | | A | A | A |
| 588 | | | A | A | A |
| 589 | | | A | A | A |
| 590 | | | A | A | A |
| 591 | | | A | A | A |
| 592 | | | A | A | A |
| 593 | | | A | A | A |
| 594 | | | A | A | A |
| 595 | | | A | A | A |
| 596 | | | A | A | A |
| 597 | | | A | A | A |
| 598 | | | A | A | A |
| 599 | | A | A | A | A |
| 600 | | | | A | A |
| 601 | | | A | A | A |
| 602 | | | A | A | A |
| 603 | | | A | A | A |
| 604 | | | A | A | A |
| 605 | | | B | A | A |
| 606 | | | A | A | A |
| 607 | | | A | A | A |
| 608 | | | A | A | A |
| 609 | | | A | A | A |
| 610 | | | A | A | A |
| 611 | | | A | A | A |
| 612 | | | A | A | A |
| 613 | | | A | A | A |
| 614 | | | A | A | A |
| 615 | | | A | A | A |
| 616 | | | A | A | A |
| 617 | | | A | A | A |
| 618 | | | A | A | A |
| 619 | | | A | A | A |
| 620 | | | A | A | A |
| 621 | | | A | A | A |
| 622 | | | A | A | A |
| 623 | | | A | A | A |
| 624 | | | A | A | A |
| 625 | | | A | A | A |
| 626 | | | A | A | A |
| 627 | | | A | A | A |
| 628 | | | A | A | A |
| 629 | | | A | A | A |
| 630 | | | A | A | A |
| 631 | | | A | A | A |
| 632 | | | A | A | A |
| 633 | | | A | A | A |
| 634 | A | A | A | A | A |
| 635 | | | A | A | A |
| 636 | | | A | A | A |
| 637 | | | A | A | A |
| 638 | | | A | A | A |
| 639 | | | A | A | A |
| 640 | | | A | A | A |
| 641 | | | A | A | A |
| 642 | | | A | A | A |
| 643 | | | A | A | A |
| 644 | | | | A | A |
| 645 | | | A | A | A |
| 646 | | | A | A | A |
| 647 | | | A | A | A |
| 648 | | | A | A | A |
| 649 | | | A | A | A |
| 650 | | | | A | A |
| 651 | | | A | A | A |
| 652 | | | A | A | A |
| 653 | | | A | A | A |
| 654 | | | A | A | A |
| 655 | | | A | A | A |
| 656 | | | A | A | A |
| 657 | | | A | A | A |
| 658 | | | A | A | A |
| 659 | | | A | A | A |
| 660 | | | A | A | A |
| 661 | | | A | A | A |
| 662 | | | A | A | A |
| 663 | | | A | A | A |
| 664 | | A | A | A | A |
| 665 | | | A | A | A |
| 666 | | | A | A | A |
| 667 | | | A | A | A |
| 668 | | | A | A | A |
| 669 | | | A | A | A |
| 670 | | | | A | A |
| 671 | | A | A | A | A |
| 672 | | | A | A | A |
| 673 | | | A | A | A |
| 674 | | | A | A | A |
| 675 | | | A | A | A |
| 676 | | | A | A | A |
| 677 | | | A | A | A |
| 678 | | | A | A | A |
| 679 | | | A | A | A |
| 680 | | | A | A | A |
| 681 | | | A | A | A |
| 682 | | | | A | A |
| 683 | | A | A | A | A |
| 684 | | | A | A | A |
| 685 | | | A | A | A |
| 686 | | | A | A | A |
| 687 | | | A | A | A |
| 688 | | | A | A | A |
| 689 | | | | A | A |
| 690 | | | A | A | A |
| 691 | | | A | A | A |
| 692 | | | A | A | A |
| 693 | | | A | A | A |
| 694 | | | A | A | A |
| 695 | | | A | A | A |
| 696 | | | A | A | A |
| 697 | | | A | A | A |
| 698 | | | A | A | A |
| 699 | | | A | A | A |
| 700 | | | A | A | A |
| 701 | | | A | A | A |
| 702 | | | A | A | A |
| 703 | | | A | A | A |
| 704 | | A | A | A | A |
| 705 | | | A | A | A |
| 706 | | | A | A | A |
| 707 | | | A | A | A |
| 708 | | | A | A | A |
| 709 | | | B | A | A |
| 710 | | | A | A | A |
| 711 | | | B | A | A |
| 712 | | | B | A | A |
| 713 | | | B | A | A |
| 714 | | | B | B | A |
| 715 | | | B | A | A |
| 716 | | | B | A | A |
| 717 | | | B | B | B |
| 718 | | | B | A | B |
| 719 | | | C | B | B |
| 720 | | | C | B | B |
| 721 | | | A | A | B |
| 722 | | | C | B | B |
| 723 | | | B | B | B |
| 724 | | | C | B | B |
| 725 | | | B | B | B |
| 726 | | | C | B | B |
| 727 | | | D | C | B |
| 728 | | | D | B | B |
| 729 | | | B | B | B |
| 730 | | D | C | B | C |
| 731 | | | D | B | C |
| 732 | | D | E | D | C |
| 733 | | | | B | D |
| 734 | | | E | D | D |
| 735 | | E | E | D | D |
| 736 | | | E | D | D |
| 737 | | | A | A | A |
| 738 | A | A | A | A | A |
| 739 | | | B | A | A |
| 740 | | | D | B | B |
| 741 | | | | | |

What is claimed is:

1. Compound of the general formula (I) 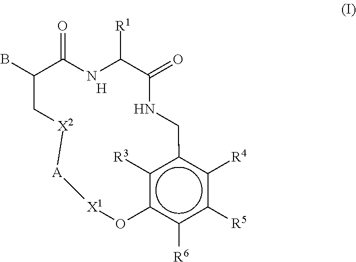
wherein
A represents —CO—N(R^{N6})—, 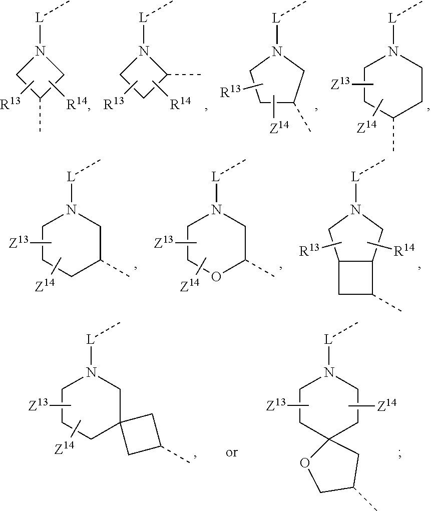
B represents —H, —NH(R²), —N(R²)(R^{N5}), 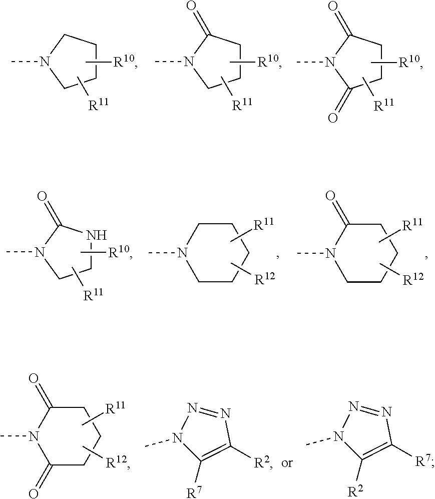
L represents —CO—, —CO—NH—, —CO—N(R^{N3})—, or —CO—O—;
R¹ represents —H, —(CH₂)ₚ—R⁷, —(CH₂)ₚ—NH—R⁷, —(CH₂)ₚ—R⁹, or —(CH₂)ₚ—NR^{N4}—R⁹;
R² represents —H, —R⁸, —R¹¹, -L¹-R¹¹, -L¹-(CH₂)ᵣ—R⁸, -L¹-R¹⁰, -L¹-(C₂H₄O)ₛ—R¹¹, -L¹-(CH₂)ₜ—O—R¹¹, -L¹-(CH₂)ₜ—NH—(CH₂)ᵣ—R⁸, -L¹-(CH₂)ₜ—O—(CH₂)ᵣ—R⁸, -L¹-(CH₂)ₜ—NHR⁸, -L¹-(CH₂)ₜ—NH—CO—R⁸, -L¹-(CH₂)ₜ—NH—SO₂—R⁸, -L¹-(CH₂)ₜ—NR^{N6}R¹⁰, -L¹-(CH₂)ₜ—O—(CH₂)ᵤ—NR^{N6}R¹⁰,
-L¹-(CH₂)ᵣ—R¹⁴, —CO—C(R¹²)(R¹³)—R¹⁰, —CO—C(R¹²)(R¹³)—R⁸, or —CO—C(R¹²)(R¹³)—(CH₂)ᵤ—R⁸;
L¹ represents a bond, —CO—, —CO₂—, —CONH—, or —SO₂—;
R³-R⁶ represent independently of each other —H, —CH₃, —OCH₃, —F, or —Cl;
or R⁵ and R⁶ form 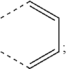
R⁸ and R⁹ represent independently of each other
C₆-C₁₄ aryl, C₁-C₁₀ heteroaryl, C₃-C₈ carbocyclyl, C₁—C heterocyclyl, C₄-C₁₁ bicyclic carbocyclyl, C₄-C₁₁ bridged carbocyclyl, C₁-C₁₀ bicyclic heterocyclyl, C₁-C₁₀ bridged heterocyclyl, C₇-C₁₆-spiroalkyl, C₅-C₁₄-spiroheterocyclyl,
wherein all afore-mentioned ring systems can be substituted with 1 to 5 substituents selected from Z¹, Z², Z³, Z⁴, Z⁵, Z⁶, Z⁷, Z⁸, Z⁹, Z¹⁰, Z¹¹, Z¹², R^{N1} and R^{N2}; and
C₁-C₁₀ heteroaryl, C₁—C heterocyclyl, C₁-C₁₀ bicyclic heterocyclyl, C₁-C₁₀ bridged heterocyclyl, C₅-C₁₄-spiroheterocyclyl ring systems contain at least one of heteroatoms N, O, and S;
said C₃-C₈ carbocyclyl, C₁—C heterocyclyl, C₁-C₁₀ bicyclic heterocyclyl, C₄-C₁₁ bridged carbocyclyl, C₁-C₁₀ bicyclic heterocyclyl, C₁-C₁₀ bridged heterocyclyl, C₇-C₁₆-spiroalkyl, C₅-C₁₄-spiroheterocyclyl ring systems can be partly saturated or unsaturated;
R⁷ and R¹⁰ represent independently of each other —H, —CH₃, —C₂H₅, —C₃H₇, —CH(CH₃)₂, —CH₂F, —CHF₂, —CF₃, —CH₂CN, —C(CH₃)₂—CN, —CH₂—C(CH₃)₂—CN, —CH₂—CF₃, —CH₂—C(CH₃)₂—NH₂, 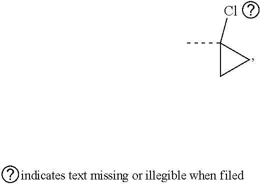
cyclo-C₃H₅, cyclo-C₄H₇, cyclo-C₅H₉, cyclo-C₆H₁₁, cyclo-C₇H₁₃, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, —C₇H₁₅, —C₈H₁₇, -Ph, —CH₂-Ph, —CH₂—CH₂-Ph, —C₂H₄—CH═CH₂, —CH₂—CH═CH—CH₃, —CH₂—C(CH₃)=CH₂, —CH(CH₃)—CH═CH₂, —C(CH₃)=CH—CH₃, —CH₂—CH═C(CH₃)₂, —CO—CH═C(CH₃)₂, —CH₂—C≡CH, —C₂H₄—C≡CH, —CH₂—C≡C—CH₃, —CH₂—OCF₃, —C₂H₄—OCF₃, —C₃H₆—OCF₃, —CH₂—OCHF₂, —C₂H₄—OCHF₂, —C₃H₆—OCHF₂, —CH₂—OCH₃, —C₂H₄—OCH₃, —C₃H₆—OCH₃, —CH₂—OC₂H₅, —C₂H₄—OC₂H₅, —C₃H₆—OC₂H₅, —CH₂—OH, —C₂H₄—OH, —C₃H₆—OH, —CH₂—COOH, —C₂H₄—COOH, —C₃H₆—COOH, —C(CH₃)₂—CN, —C(CH₃)₂—OH, —C(CH₃)₂—CH₂—OH, —C(C₂H₅)₂—CH₂—OH, —C(CH₂—OH)₂—CH₃, —C(CH₂—OH)₂—C₂H₅, —C(CH₃)₂—CH₂—SH, —C(C₂H₅)₂—CH₂—SH, —C(CH₂—SH)₂—CH₃, —CO—O—C(CH₃)₃, or —C(CH₂—SH)₂—C₂H₅;
R¹¹ represents —H, —CH₃, —C₂H₅, —C₃H₇, —CH(CH₃)₂, —CH₂F, —CHF₂, —CF₃, —CH₂CN, —C(CH₃)₂—CN, —CH₂—C(CH₃)₂—CN, —CH₂—CF₃, —CH₂—C(CH₃)₂—NH₂, 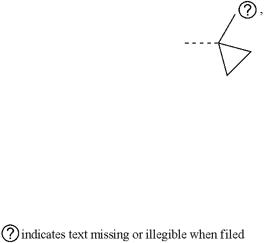
cyclo-C₃H₅, cyclo-C₄H₇, cyclo-C₅H₉, cyclo-C₆H₁₁, cyclo-C₇H₁₃, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, —C₇H₁₅, —C₈H₁₇, -Ph, —CH₂-Ph, —CH₂—CH₂-Ph, —CH═CH₂, —CH₂—CH═CH₂, —C(CH₃)=CH₂, —CH═CH—CH₃, —C(CH₃)=CH—CH₃, —CH═C(CH₃)₂, —C(CH₃)═C(CH₃)₂, —C₂H₄—CH═CH₂, —CH₂—CH═CH—CH₃, —CH₂—C(CH₃)=CH₂, —CH(CH₃)—CH═CH₂, —C(CH₃)=CH—CH₃, —CH₂—CH═C(CH₃)₂, —C≡CH, —CH₂—C≡CH, —C₂H₄—C≡CH, —CH₂—C≡C—CH₃, —C≡C—CH₃, —C≡C—C₂H₅, —CH₂—OCF₃, —C₂H₄—OCF₃, —C₃H₆—OCF₃, —CH₂—OCHF₂, —C₂H₄—OCHF₂, —C₃H₆—OCHF₂, —CH₂—OCH₃, —C₂H₄—OCH₃, —C₃H₆—OCH₃, —CH₂—OC₂H₅, —C₂H₄—OC₂H₅, —C₃H₆—OC₂H₅, —CH₂—OH, —C₂H₄—OH, —C₃H₆—OH, —C(CH₃)₂—CH₂—OH, —C(C₂H₅)₂—CH₂—OH, —C(CH₂—OH)₂—CH₃, —C(CH₂—OH)₂—C₂H₅, —C(CH₃)₂—CH₂—SH, —C(C₂H₅)₂—CH₂—SH, —C(CH₂—SH)₂—CH₃, or —C(CH₂—SH)₂—C₂H₅;
R¹² and R¹³ represent independently of each other —H, —CH₃, —C₂H₅, —C₃H₇, —CH(CH₃)₂, -Ph, —CH₂-Ph, —COOH, —NH₂, —NHCO₂(CCH₃)₃, —CH₂—NH₂, —CHF₂, —CF₃, —F, —OCF₃, —OCHF₂, —OH, —OCH₃, —OC₂H₅, —OC₃H₇, or —OCH(CH₃)₂; or
R¹² and R¹³ form together 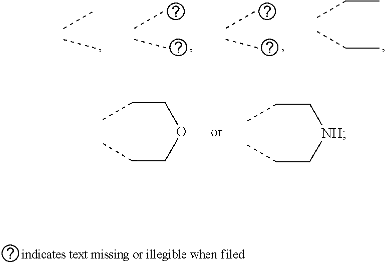
R¹⁴ represents 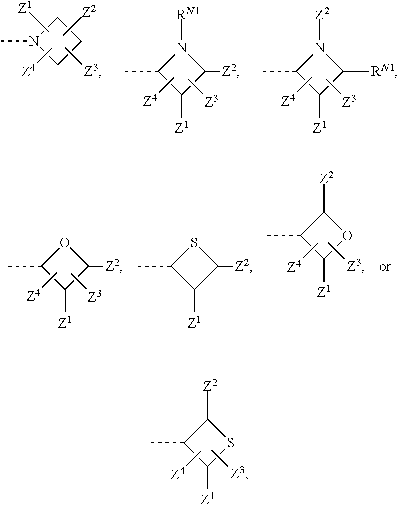
R¹⁵ and R¹⁶ represent independently of each other —X³-L²-R¹⁷, or —(OCH₂CH₂)_{w}—R¹⁷;
L² represents —(CH₂)ᵥ—, —(CH₂CH₂—O)_{w}—CH₂—, or —(CH₂CH₂—O)_{w}—CH₂CH₂—;
R¹⁷ represents —OH, —SH, —SO₃H, —NH₂, or —CO₂H;
R^{N1}, R^{N2}, R^{N3}, and R^{N4} represent independently of each other —R¹⁵, —H, —CH₃, —C₂H₅, —C₃H₇, —CH(CH₃)₂, —CHF₂, —CF₃, 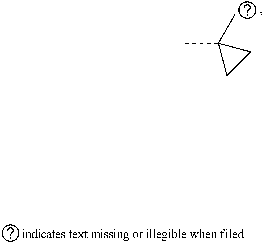
cyclo-C₃₁—H₅, cyclo-C₄H₇, cyclo-C₅H₉, cyclo-C₆H₁₁,
cyclo-C₇H₁₃, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, —C₇H₁₅, —C₈H₁₇, -Ph, —CH₂-Ph, —CH₂—CH₂-Ph, —C₂H₄—CH═CH₂, —CH₂—CH═CH—CH₃, —CH₂—C(CH₃)=CH₂, —CH(CH₃)—CH═CH₂, —CH₂—CH═C(CH₃)₂, —CH₂—C≡CH, —C₂H₄—C≡CH, —CH₂—C≡C—CH₃, —COCH₃, —COC₂H₅, —COC₃H₇, —CO-cyclo-C₃H₅, —COCH(CH₃)₂, —COC(CH₃)₃, —COOH, —COOCH₃, —COOC₂H₅, —COOC₃H₇, —COO-cyclo-C₃H₅, —COOCH(CH₃)₂, —COOC(CH₃)₃, —COOCH₂Ph, —CONH₂, —CONHCH₃, —CONHC₂H₅, —CONHC₃H₇, —CONH-cyclo-C₃H₅, —CONH[CH(CH₃)₂], —CONH[C(CH₃)₃], —CON(CH₃)₂, —CON(C₂H₅)₂, —CON(C₃H₇)₂, —CON(cyclo-C₃H₅)₂, —CON[CH(CH₃)₂]₂, —CON[C(CH₃)₃]₂, or —SO₃H;
R^{N5} and R^{N6} represent independently of each other —H, —CH₃, —C₂H₅, —C₃H₇, —CH(CH₃)₂, cyclo-C₃H₅, —COOC(CH₃)₃, or —COOCH₂Ph;
X¹ represents —(CH₂)ₘ—;
X² represents —(CH₂)ₙ—;
X³ represents a bond, —O—, —NH—, or —S—;
Z¹-Z¹⁴ represent independently of each other 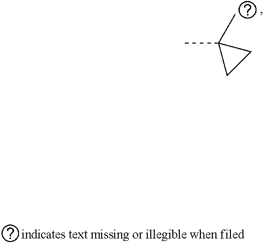
cyclo-C₃H₅, cyclo-C₄H₇, cyclo-C₅H₉, cyclo-C₆H₁₁, cyclo-C₇H₁₃,
—R¹⁶, —H, —OH, —OCH₃, —OC₂H₅, —OC₃H₇, —O-cyclo-C₃H₅, —OCH(CH₃)₂, —OC(CH₃)₃, —OC₄H₉, —O-cyclo-C₄H₇, —O-cyclo-C₅H₉, —O-cyclo-C₆H₁₁, —OCH₂CH(CH₃)₂, —OCH₂-cyclo-C₃H₅, —OCH₂-cyclo-C₄H₇, —OCH₂-cyclo-C₅H₉, —OCH₂-cyclo-C₆H₁₁, —OPh, —OCH₂-Ph, —OCPh₃, —CH₂—OCH₃, —C₂H₄—OCH₃, —C₃H₆—OCH₃, —CH₂—OC₂H₅, —C₂H₄—OC₂H₅, —C₃H₆—OC₂H₅, —CH₂—OC₃H₇, —C₂H₄—OC₃H₇, —C₃H₆—OC₃H₇, —CH₂—O-cyclo-C₃H₅, —C₂H₄—O-cyclo-C₃H₅, —C₃H₆—O-cyclo-C₃H₅, —CH₂—OCH(CH₃)₂, —C₂H₄—OCH(CH₃)₂, —C₃H₆—OCH(CH₃)₂, —CH₂—OC(CH₃)₃, —C₂H₄—OC(CH₃)₃, —C₃H₆—OC(CH₃)₃, —CH₂—OC₄H₉, —C₂H₄—OC₄H₉, —C₃H₆—OC₄H₉, —CH₂—OPh, —C₂H₄—OPh, —C₃H₆—OPh, —CH₂—OCH₂-Ph, —C₂H₄—OCH₂-Ph, —C₃H₆—OCH₂-Ph, —SH, —SCH₃, —SC₂H₅, —SC₃H₇, —S-cyclo-C₃H₅, —SCH(CH₃)₂, —SC(CH₃)₃, —F, —Cl, —Br, —I, —CN, —COCH₃, —COC₂H₅, —COC₃H₇, —CO-cyclo-C₃H₅, —COCH(CH₃)₂, —COC(CH₃)₃, —COOH, —COOCH₃, —COOC₂H₅, —COOC₃H₇, —COO-cyclo-C₃H₅, —COOCH(CH₃)₂, —COOC(CH₃)₃, —OOC—CH₃, —OOC—C₂H₅, —OOC—C₃H₇, —OOC-cyclo-C₃H₅, —OOC—CH(CH₃)₂, —OOC—C(CH₃)₃, —CONH₂, —CONHCH₃, —CONHC₂H₅, —CONHC₃H₇, —CONH-cyclo-C₃H₅, —CONH[CH(CH₃)₂], —CONH[C(CH₃)₃], —CON(CH₃)₂, —CON(C₂H₅)₂, —CON(C₃H₇)₂, —CON(cyclo-C₃H₅)₂, —CON[CH(CH₃)₂]₂, —CON[C(CH₃)₃]₂, —NHCOCH₃, —NHCOC₂H₅, —NHCOC₃H₇, —NHCO-cyclo-C₃H₅, —NHCO—CH(CH₃)₂, —NHCO—C(CH₃)₃, —NHCO—CH(NH₂)CH₂—COOH, —NHCO—CH(NH₂)CH₂CH₂—COOH, —NHCO—OCH₃, —NHCO—OC₂H₅, —NHCO—OC₃H₇, —NHCO—O-cyclo-C₃H₅, —NHCO—OCH(CH₃)₂, —NHCO—OC(CH₃)₃, —NH₂, —NHCH₃, —NHC₂H₅, —NHC₃H₇, —NH-cyclo-C₃H₅, —NHCH(CH₃)₂, —NHC(CH₃)₃, —N(CH₃)₂, —N(C₂H₅)₂, —N(C₃H₇)₂, —N(cyclo-C₃H₅)₂, —N[CH(CH₃)₂]₂, —N[C(CH₃)₃]₂, —SOCH₃, —SOC₂H₅, —SOC₃H₇, —SO-cyclo-C₃H₅, —SOCH(CH₃)₂, —SOC(CH₃)₃, —SO₂CH₃, —SO₂C₂H₅, —SO₂C₃H₇, —SO₂-cyclo-C₃H₅, —SO₂CH(CH₃)₂, —SO₂C(CH₃)₃, —SO₃H, —SO₃CH₃, —SO₃C₂H₅, —SO₃C₃H₇, —SO₃-cyclo-C₃H₅, —SO₃CH(CH₃)₂, —SO₃C(CH₃)₃, —SO₂NH₂, —SO₂NHCH₃, —SO₂NHC₂H₅, —SO₂NHC₃H₇, —SO₂NH-cyclo-C₃H₅, —SO₂NHCH(CH₃)₂, —SO₂NHC(CH₃)₃, —SO₂N(CH₃)₂, —SO₂N(C₂H₅)₂, —SO₂N(C₃H₇)₂, —SO₂N(cyclo-C₃H₅)₂, —SO₂N[CH(CH₃)₂]₂, —SO₂N[C(CH₃)₃]₂, —O—S(═O)CH₃, —O—S(═O)C₂H₅, —O—S(═O)C₃H₇, —O—S(═O)-cyclo-C₃H₅, —O—S(═O)CH(CH₃)₂, —O—S(═O)C(CH₃)₃, —S(═O)(═NH)CH₃, —S(═O)(═NH)C₂H₅, —S(═O)(═NH)C₃H₇, —S(═O)(═NH)-cyclo-C₃H₅, —S(═O)(═NH)CH(CH₃)₂, —S(═O)(═NH)C(CH₃)₃, —NH—SO₂—CH₃, —NH—SO₂—C₂H₅, —NH—SO₂—C₃H₇, —NH—SO₂-cyclo-C₃H₅, —NH—SO₂—CH(CH₃)₂, —NH—SO₂—C(CH₃)₃, —O—SO₂—CH₃, —O—SO₂—C₂H₅, —O—SO₂—C₃H₇, —O—SO₂-cyclo-C₃H₅, —O—SO₂—CH(CH₃)₂, —O—SO₂—C(CH₃)₃, —OCH₂F, —OCHF₂, —OCF₃, —CH₂—OCF₃, —C₂H₄—OCF₃, —C₃H₆—OCF₃, —CH₂—OCHF₂, —C₂H₄—OCHF₂, —C₃H₆—OCHF₂, —OC₂F₅, —CH₂—OC₂F₅, —C₂H₄—OC₂F₅, —C₃H₆—OC₂F₅, —O—COOCH₃, —O—COOC₂H₅, —O—COOC₃H₇, —O—COO-cyclo-C₃H₅, —O—COOCH(CH₃)₂, —O—COOC(CH₃)₃, —NH—CO—NH₂, —NH—CO—NHCH₃, —NH—CO—NHC₂H₅, —NH—CO—NHC₃H₇, —NH—C(═NH)—NH₂, —NH—CO—N(C₃H₇)₂, —NH—CO—NH[CH(CH₃)₂], —NH—CO—NH[C(CH₃)₃], —NH—CO—N(CH₃)₂, —NH—CO—N(C₂H₅)₂, —NH—CO—NH-cyclo-C₃H₅, —NH—CO—N(cyclo-C₃H₅)₂, —NH—CO—N[CH(CH₃)₂]₂, —NH—C(═NH)—NHCH₃, —NH—C(═NH)—NHC₂H₅, —NH—C(═NH)—NHC₃H₇, —O—CO—NH-cyclo-C₃H₅, —NH—C(═NH)—NH-cyclo-C₃H₅, —NH—C(═NH)—NH[CH(CH₃)₂], —O—CO—NH[CH(CH₃)₂], —NH—C(═NH)—NH[C(CH₃)₃], —NH—C(═NH)—N(CH₃)₂, —NH—C(═NH)—N(C₂H₅)₂, —NH—C(═NH)—N(C₃H₇)₂, —NH—C(═NH)—N(cyclo-C₃H₅)₂, —O—CO—NHC₃H₇, —NH—C(═NH)—N[CH(CH₃)₂]₂, —NH—C(═NH)—N[C(CH₃)₃]₂, —O—CO—NH₂, —O—CO—NHCH₃, —O—CO—NHC₂H₅, —O—CO—NH[C(CH₃)₃], —O—CO—N(CH₃)₂, —O—CO—N(C₂H₅)₂, —O—CO—N(C₃H₇)₂, —O—CO—N(cyclo-C₃H₅)₂, —O—CO—N[CH(CH₃)₂]₂, —O—CO—N[C(CH₃)₃]₂, —O—CO—OCH₃, —O—CO—OC₂H₅, —O—CO—OC₃H₇, —O—CO—O-cyclo-C₃H₅, —O—CO—OCH(CH₃)₂, —O—CO—OC(CH₃)₃, —CH₂F, —CHF₂, —CF₃, —CH₂—CH₂F, —CH₂—CHF₂, —CH₂—CF₃, cyclo-C₈H₁₅, -Ph, —CH₂-Ph, —CH₂—CH₂-Ph, —CH═CH-Ph, —CPh₃, —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, —C₇H₁₅, —C₈H₁₇, —C₃H₆—CH(CH₃)₂, —C₂H₄—CH(CH₃)—C₂H₅, —CH(CH₃)—C₄H₉, —CH₂—CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, —CH(CH₃)—C(CH₃)ₛ, —CH═CH₂, —CH₂—CH═CH₂, —C(CH₃)=CH₂, —CH═CH—CH₃, —C₂H₄—CH═CH₂, —CH₂—CH═CH—CH₃, —CH═CH—C₂H₅, —CH₂—C(CH₃)=CH₂, —CH(CH₃)—CH═CH, —CH═C(CH₃)₂, —C(CH₃)=CH—CH₃, —CH═CH—CH═CH₂, —C₃H₆—CH═CH₂, —C₂H₄—CH═CH—CH₃, —CH₂—CH═CH—C₂H₅, —CH═CH—C₃H₇, —CH═CH—CH═CH—CH₃, —C₂H₄—C(CH₃)=CH₂, —CH₂—CH(CH₃)—CH═CH₂, —CH(CH₃)—CH₂—CH═CH₂, —CH₂—CH═C(CH₃)₂, —CH₂—C(CH₃)=CH—CH₃, —CH(CH₃)—CH═CH—CH₃, —CH═CH—CH(CH₃)₂, —CH═C(CH₃)—C₂H₅, —C(CH₃)=CH—C₂H₅, —C(CH₃)═C(CH₃)₂, —C(CH₃)₂—CH═CH₂, —CH(CH₃)—C(CH₃)=CH₂, —C₄H₈—CH═CH₂, —C₃H₆—CH═CH—CH₃, —C₂H₄—CH═CH—C₂H₅, —CH₂—CH═CH—C₃H₇, —CH═CH—C₄H₉, —C₃H₆—C(CH₃)=CH₂, —C₂H₄—CH(CH₃)—CH═CH₂, —CH₂—CH(CH₃)—CH₂—CH═CH₂, —C₂H₄—CH═C(CH₃)₂, —CH(CH₃)—C₂H₄—CH═CH₂, —C₂H₄—C(CH₃)=CH—CH₃, —CH₂—CH(CH₃)—CH═CH—CH₃, —CH(CH₃)—CH₂—CH═CH—CH₃, —CH₂—CH═CH—CH(CH₃)₂, —CH₂—CH═C(CH₃)—C₂H₅, —CH₂—C(CH₃)=CH—C₂H₅, —CH(CH₃)—CH═CH—C₂H₅, —CH═CH—CH₂—CH(CH₃)₂, —CH═CH—CH(CH₃)—C₂H₅, —CH═C(CH₃)—C₃H₇, —C(CH₃)=CH—C₃H₇, —CH₂—CH(CH₃)—C(CH₃)=CH₂, —C[C(CH₃)₃]=CH₂, —CH(CH₃)—CH₂—C(CH₃)=CH₂, —CH(CH₃)—CH(CH₃)—CH═CH₂, —CH═CH—C₂H₄—CH═CH₂, —C(CH₃)₂—CH₂—CH═CH₂, —CH₂—C(CH₃)═C(CH₃)₂, —CH(CH₃)—CH═C(CH₃)₂, —C(CH₃)₂—CH═CH—CH₃, —CH═CH—CH₂—CH═CH—CH₃, —CH(CH₃)—C(CH₃)=CH—CH₃, —CH═C(CH₃)—CH(CH₃)₂, —C(CH₃)=CH—CH(CH₃)₂, —C(CH₃)═C(CH₃)—C₂H₅, —CH═CH—C(CH₃)ₛ, —C(CH₃)₂—C(CH₃)=CH₂, —CH(C₂H₅)—C(CH₃)=CH₂, —C(CH₃)(C₂H₅)—CH═CH₂, —CH(CH₃)—C(C₂H₅)=CH₂, —CH₂—C(C₃H₇)=CH₂, —CH₂—C(C₂H₅)=CH—CH₃, —CH(C₂H₅)—CH═CH—CH₃, —C(C₄H₉)=CH₂, —C(C₃H₇)=CH—CH₃, —C(C₂H₅)=CH—C₂H₅, —C(C₂H₅)═C(CH₃)₂, —C[CH(CH₃)(C₂H₅)]=CH₂, —C[CH₂—CH(CH₃)₂]=CH₂, —C₂H₄—CH═CH—CH═CH₂, —CH₂—CH═CH—CH₂—CH═CH₂, —C₃H₆—C≡C—CH₃, —CH₂—CH═CH—CH═CH—CH₃, —CH═CH—CH═CH—C₂H₅, —CH(CH₃)—CH₂—C≡CH, —CH(CH₃)—C≡C—CH₃, —C₂H₄—CH(CH₃)—C≡CH, —CH═CH—CH═C(CH₃)₂, —CH₂—CH(CH₃)—CH₂—C≡CH, —CH═CH—C(CH₃)=CH—CH₃, —CH═C(CH₃)—CH═CH—CH₃, —CH₂—CH(CH₃)—C≡CH, —C(CH₃)=CH—CH═CH—CH₃, —C≡CH, —C≡C—CH₃, —CH₂—C≡CH, —C₂H₄—C≡CH, —CH₂—C≡C—CH₃, —C≡C—C₂H₅, —C₃H₆—C≡CH, —C₂H₄—C≡C—CH₃, —CH₂—C≡C—C₂H₅, —C≡C—C₃H₇, —CH(CH₃)—C≡CH, —C₄H₈—C≡CH, —C₂H₄—C≡C—C₂H₅, —CH₂—C≡C—C₃H₇, —C≡C—C₄H₉, —C≡C—CH₂—CH(CH₃)₂, —CH(CH₃)—C₂H₄—C≡CH, —CH₂—CH(CH₃)—C≡C—CH₃, —C(CH₃)(C₂H₅)—C≡CH, —CH(CH₃)—CH₂—C≡C—CH₃, —CH(CH₃)—C≡C—C₂H₅, —CH₂—C≡C—CH(CH₃)₂, —C≡C—CH(CH₃)—C₂H₅, —CH₂—C≡C—C≡C—CH₃, —CH(C₂H₅)—C≡C—CH₃, —C(CH₃)₂—C≡C—CH₃, —CH(C₂H₅)—CH₂—C≡CH, —CH₂—CH(C₂H₅)—C≡CH, —C(CH₃)₂—CH₂—C≡CH, —CH₂—C(CH₃)₂—C≡CH, —CH(CH₃)—CH(CH₃)—C≡CH, —CH(C₃H₇)—C≡CH, —CH₂—CH(C≡CH)₂, —C≡C—C≡CH, —CH₂—C≡C—C≡CH, —C≡C—C≡C—CH₃, —CH(C≡CH)₂, —C₂H₄—C≡C—C≡CH, —CH₂—C≡C—CH₂—C≡CH, —C≡C—C₂H₄—C≡CH, —C≡C—C(CH₃)₃, —C≡C—CH₂—C≡C—CH₃, —C≡C—C≡C—C₂H₅, 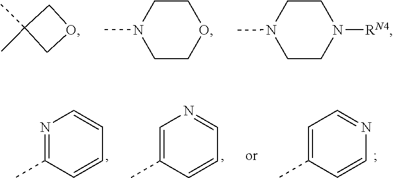
Z³ and Z⁴ may form together 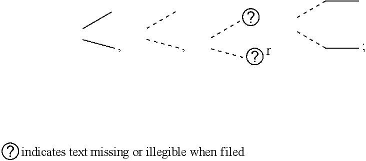
Z¹³ and Z¹⁴ may form together 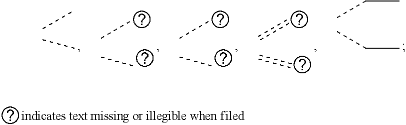
m is an integer selected from 0, 1, 2, 3, 4, 5, or 6;
n is an integer selected from 0, 1, 2, 3, 4, 5, or 6;
p is an integer selected from 0, 1, 2, 3, 4, 5, or 6;
r is an integer selected from 0, 1, 2, 3, or 4;
s is an integer selected from 1, 2, 3, 4, 5, 6, 7, 8, 9, or 10;
t is an integer selected from 1, 2, 3, or 4;
u is an integer selected from 1, 2, 3, or 4;
v is an integer selected from 0, 1, 2, 3, 4, 5, or 6;
w is an integer selected from 0, 1, 2, 3, 4, 5 or 6;
or an enantiomer, a stereoisomeric form, a mixture of enantiomers, a diastereomer, a mixture of diastereomers, a hydrate, a solvate, an acid salt form, a tautomer, a racemate of the above mentioned compounds, or a pharmaceutically acceptable salt thereof.

2. The compound according to claim 1, wherein R⁸ and R⁹ represent independently of each other: 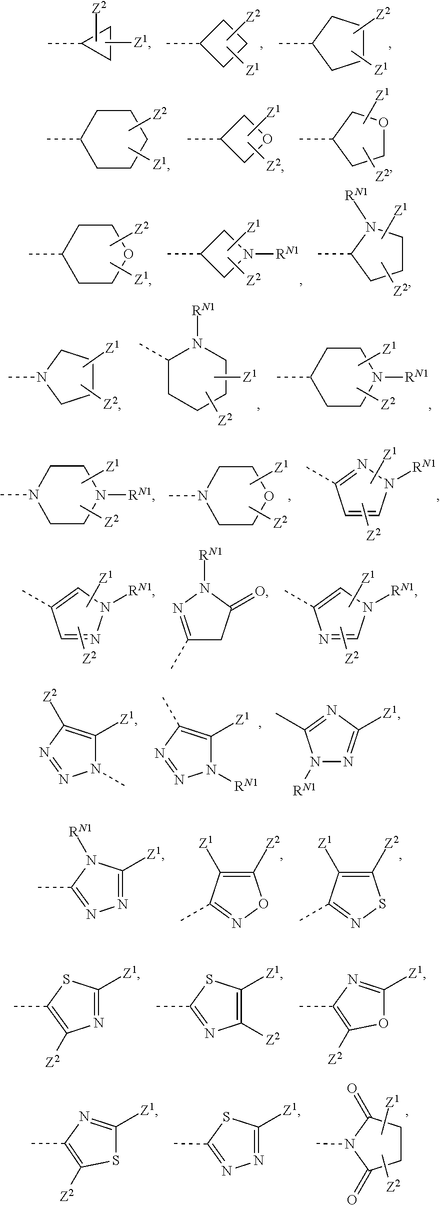 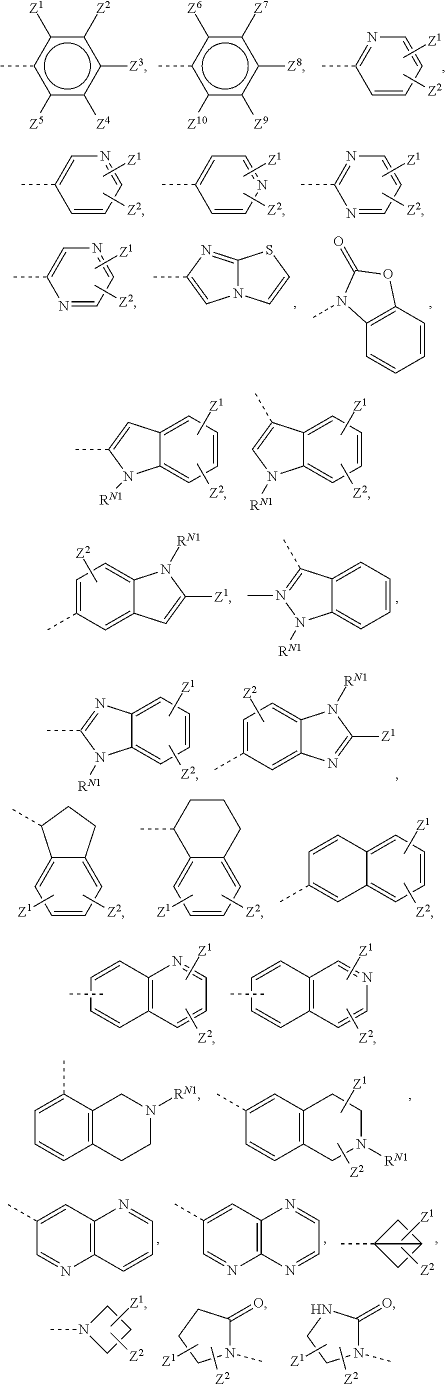 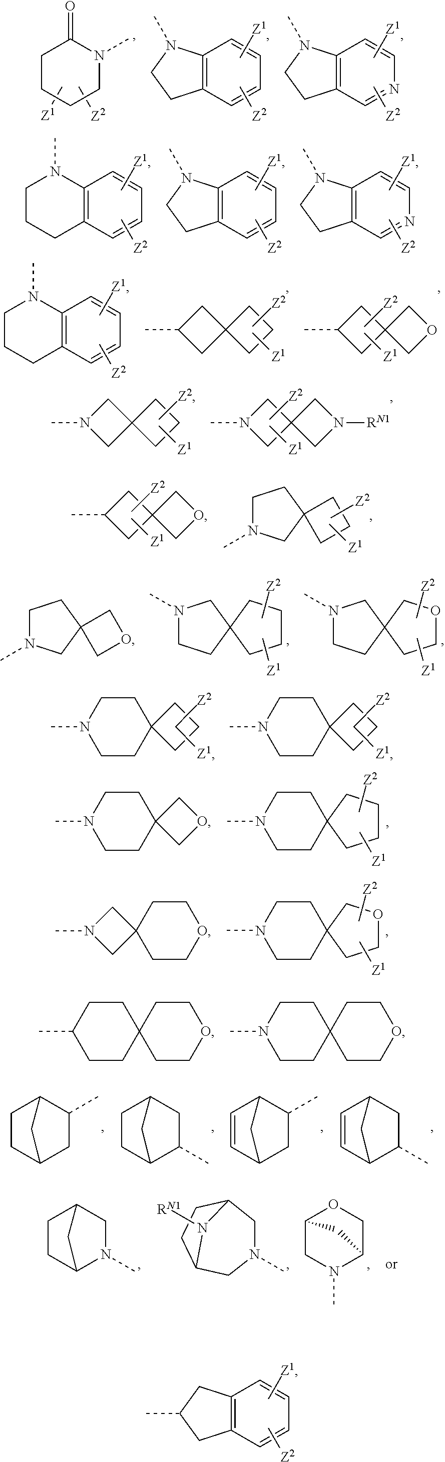
wherein R^{N1}, Z¹, Z², Z³, Z⁴, Z⁵, Z⁶, Z⁷, Z⁸, Z⁹, and Z¹⁰ have the same meanings as defined in claim 1.

3. The compound according to claim 1, wherein R⁸ represents 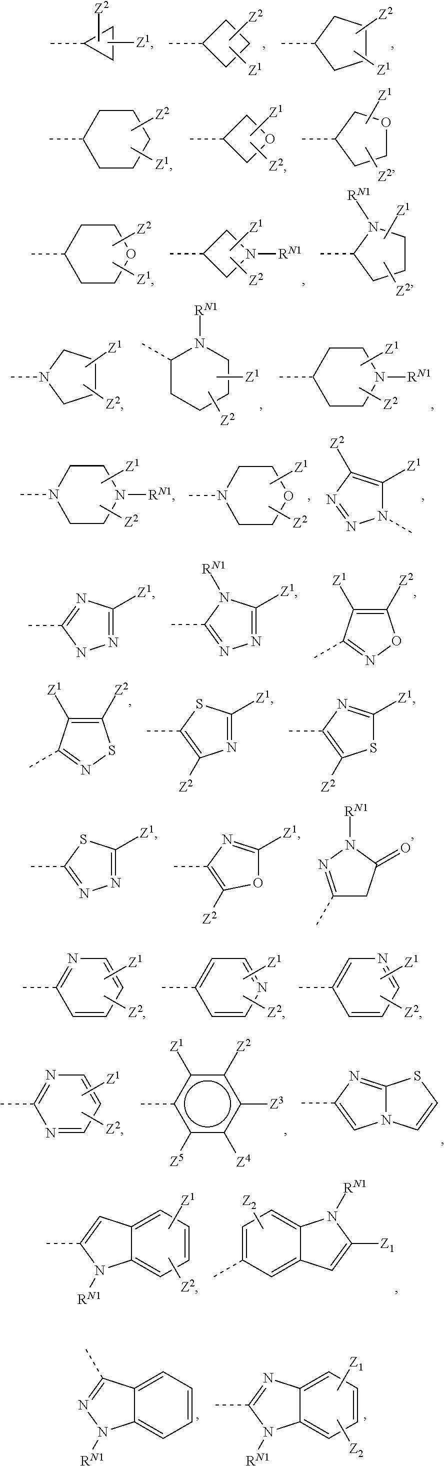 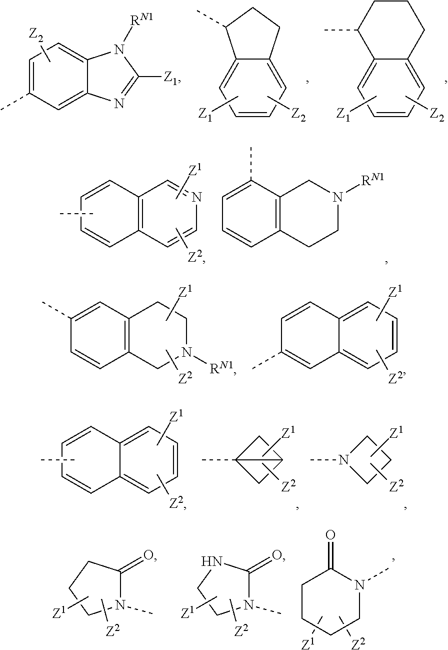 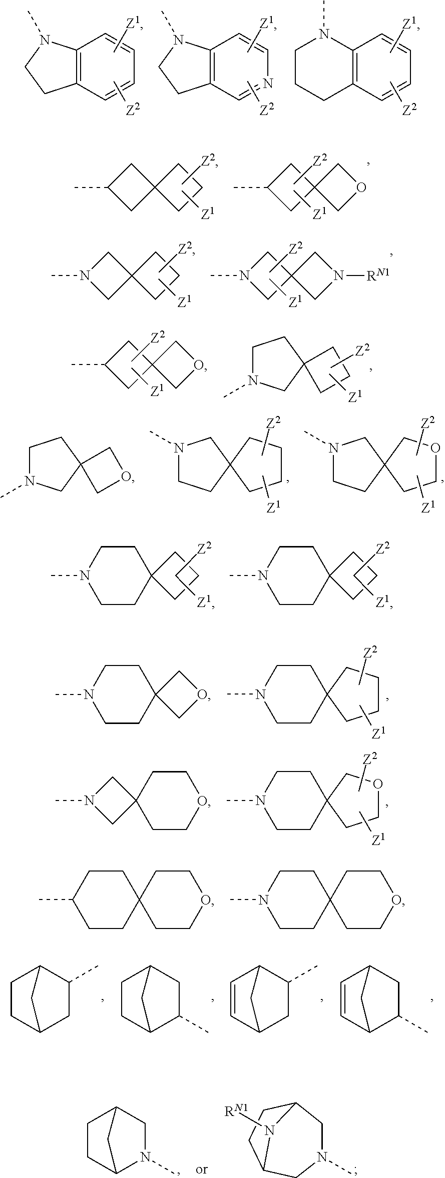
wherein R^{N1}, Z¹, Z², Z⁶, Z⁷, Z⁸, Z⁹, and Z¹⁰ have the same meanings as defined in claim 1.

4. The compound according to claim 1, wherein R⁹ represents 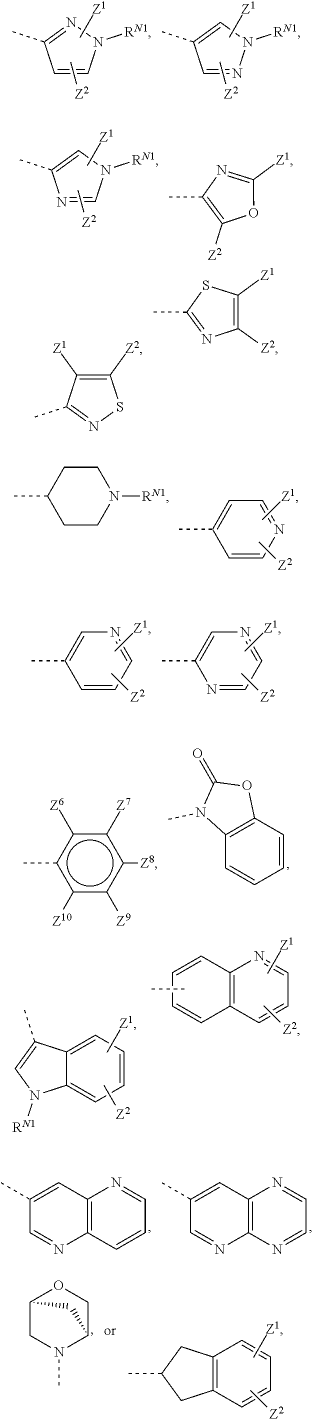
wherein R^{N1}, Z¹, Z², Z⁶, Z⁷, Z⁸, Z⁹, and Z¹⁰ have the same meanings as defined in claim 1.

5. The compound according to claim 1, wherein A represents
—CO—NH—, —CO—N(CH₃)—,

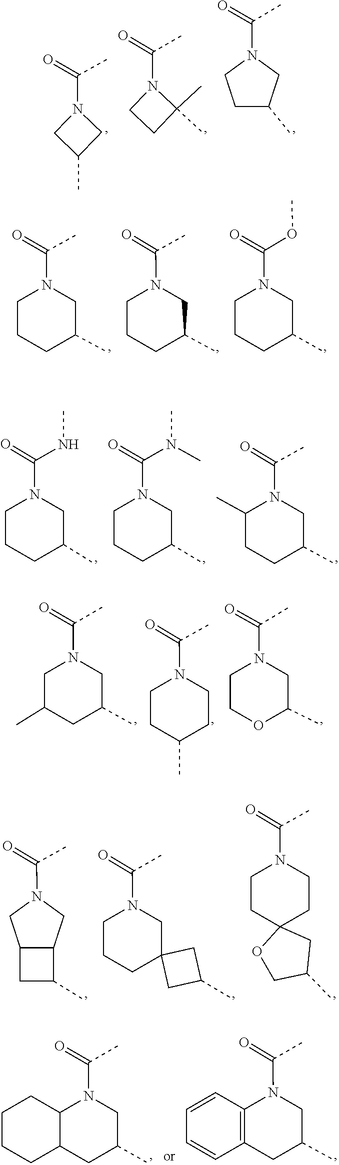

6. The compound according to claim 1, wherein
—X₂-A-X₁— represents

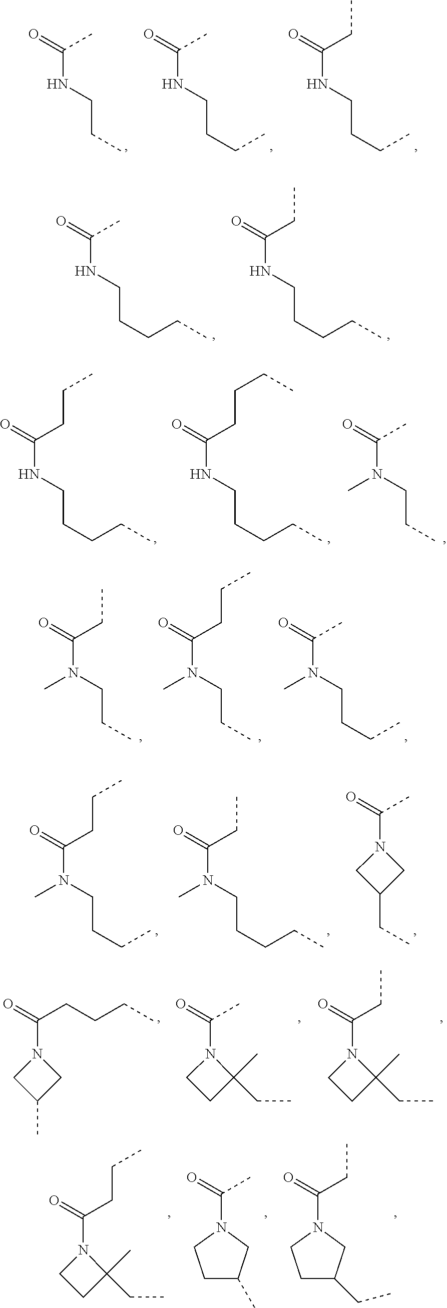

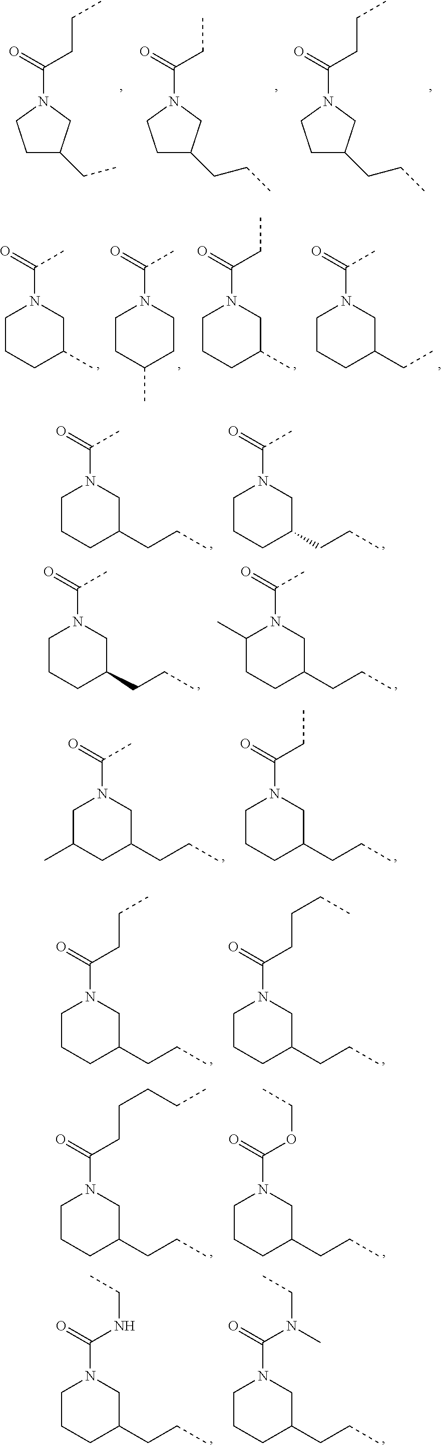

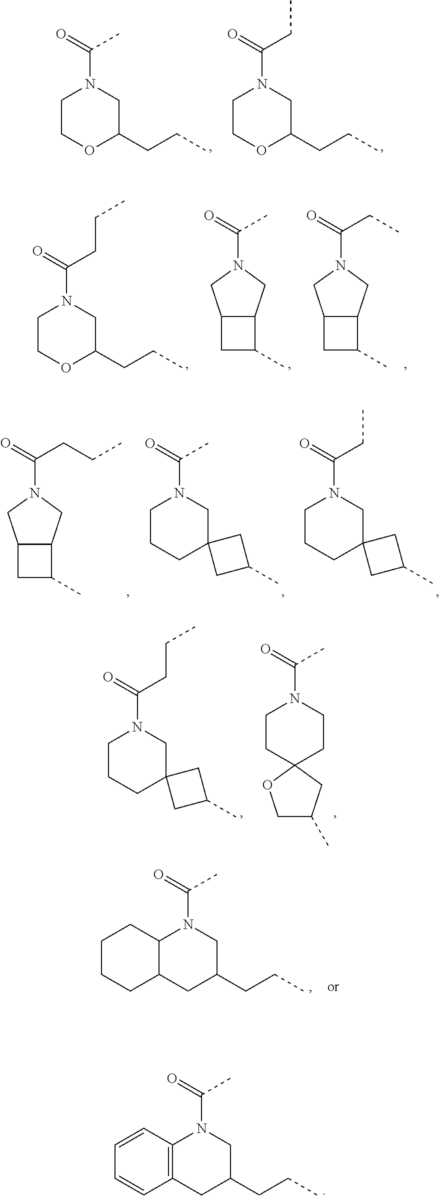

7. The compound according to claim 1, wherein
R¹ represents —CH₃, —CH₂CH₂—CH(CH₃)₂, —CH₂CH₂—C≡CH,

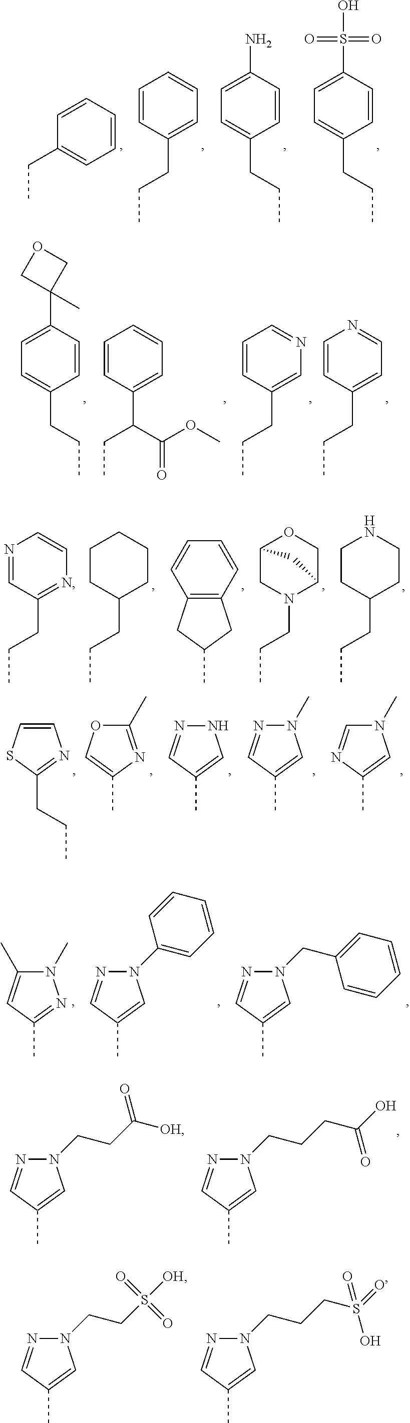

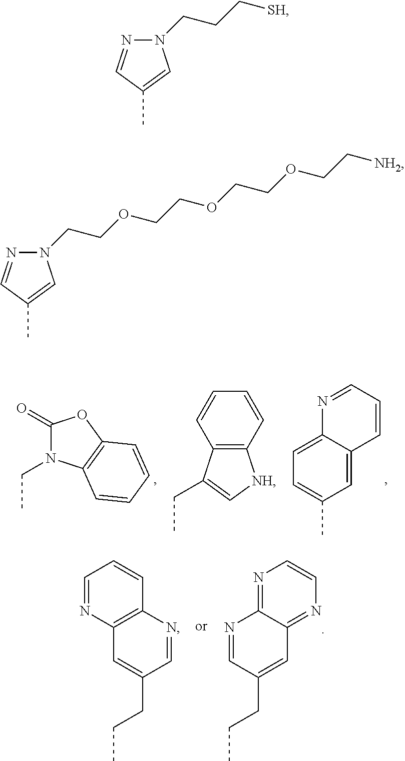

8. The compound according to claim 1, wherein
B represents —H, —NH₂, —NHCOCH₃, —NHCOC(CH₃)₃, —NHCOC(CN)(CH₃)₂, —NHCOCH═CH₂, —NHCOCH₂C(CH₃)₃, —NHCOPh, —NHCOCH₂Ph, —NHCOCH₂—NH(CH₃), —NHCOCH₂—N(CH₃)CO₂tBu, —NHCOC(CH₃)₂CH₂OH, —NHCOC(CH₃)₂CH₂SH, —NHCOC(CH₃)₂CH₂NH₂, —NHCOC(CH₃)₂CH₂F, —NHCOC(CH₃)₂CF₃, —NHCOCF₂CH₂OH, —NHCOCF₂CH₂NH₂, —NHCOC(CH₂CH₃)₂CH₂OH, —NHCOC(CH₃)(CH₂OH)₂, —NHCOCH₂OCH₂CH₂—NH(CH₃), —NHCOCH₂OCH₂CH₂—N(CH₃)CO₂tBu, —NHCO(CH₂CH₂O)₂CH₃, —NHCO₂CH₂CH₃, —NHCO₂(CH₂CH₂O)₃CH₃, —NHCO₂(CH₂CH₂O)₅CH₃, —NHCO₂(CH₂CH₂O)₇CH₃, —NHCO₂CH₂Ph —NHCONHCH₂CH₃, —NHSO₂CH₃, —NHSO₂CH═CH₂, —NHSO₂CH₂Ph, —NHSO₂CH₂CH₂Ph, —NHSO₂CH₂CH₂CH₂Ph, —NHSO₂CH₂CF₃, —NHCH₂CF₃, —NHCH₂(CH₃)₂NH₂, —NHCH₂(CH₃)₂CH₂OH,

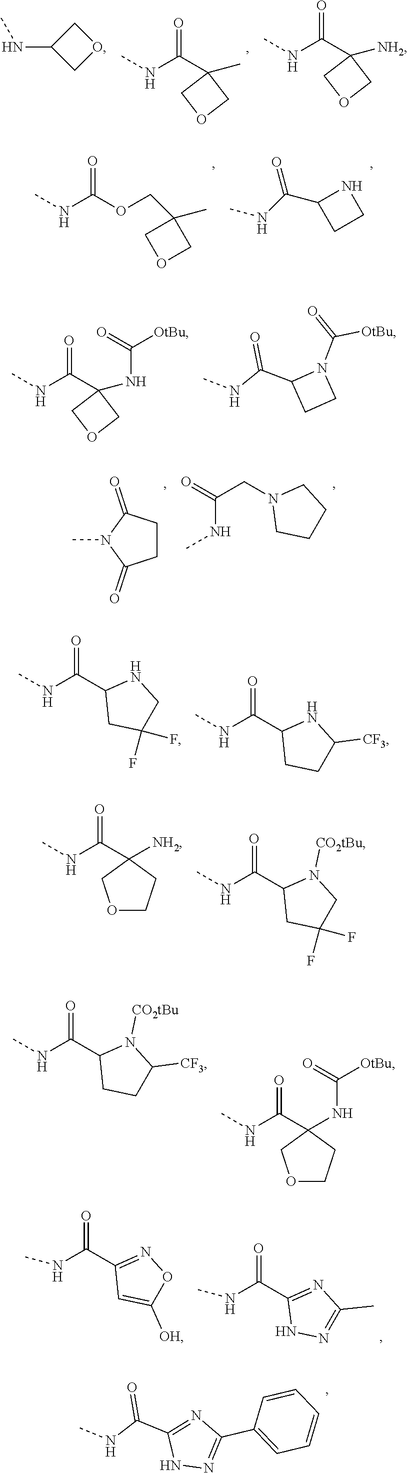

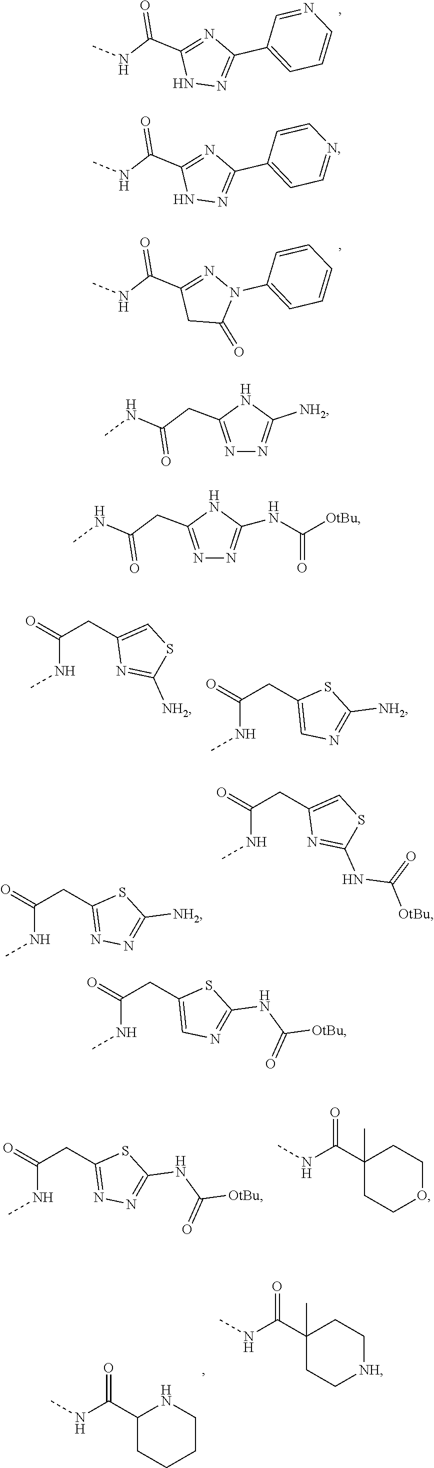

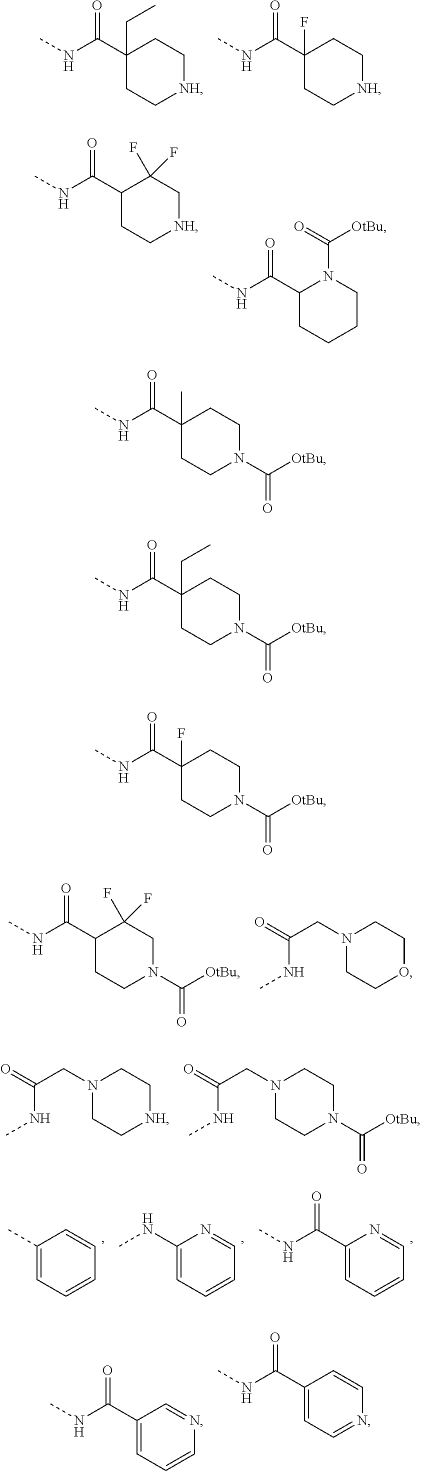

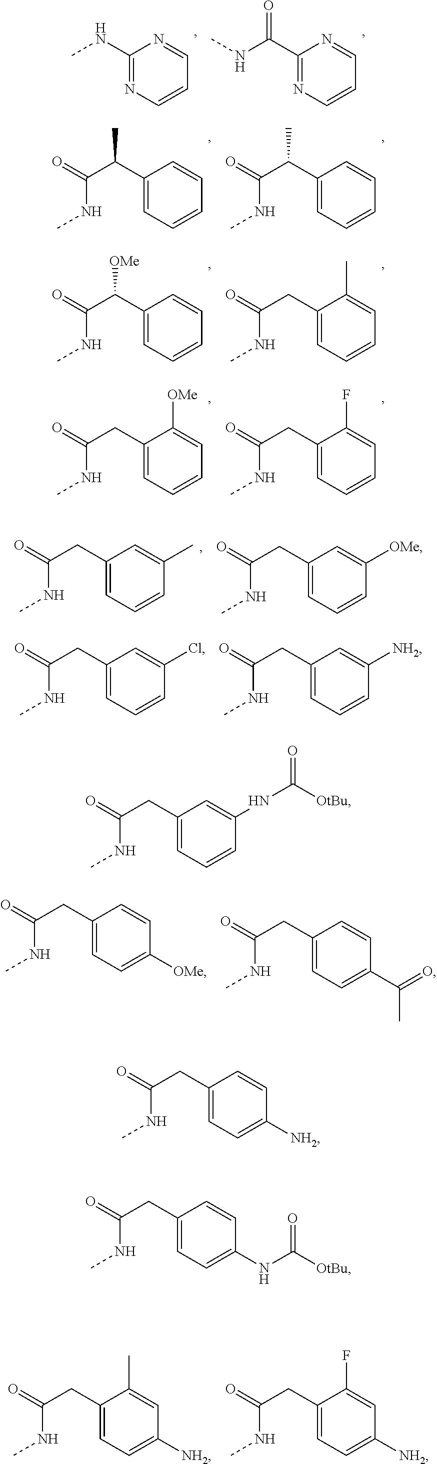

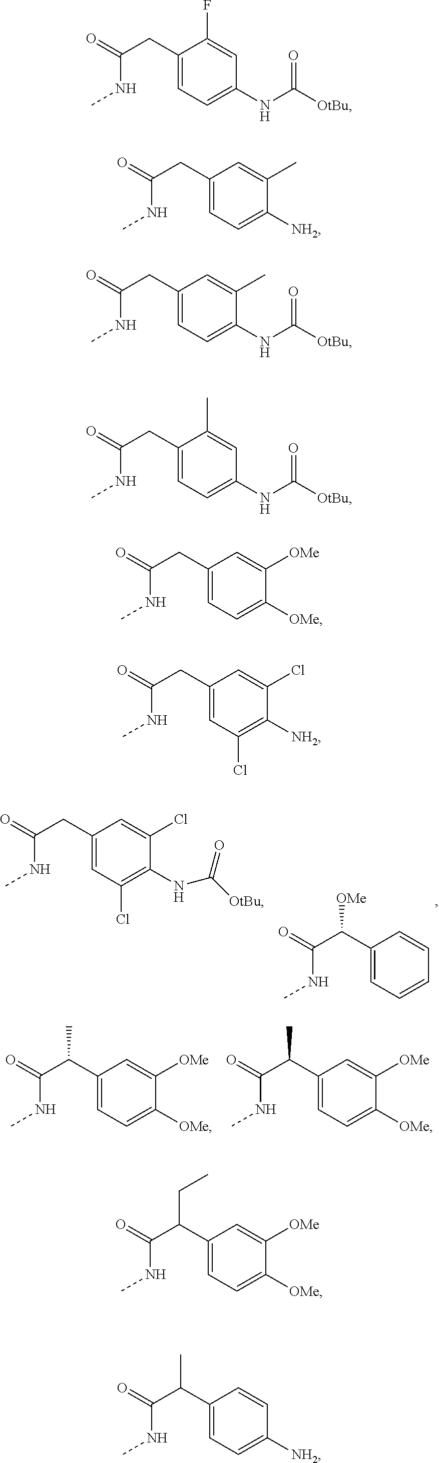

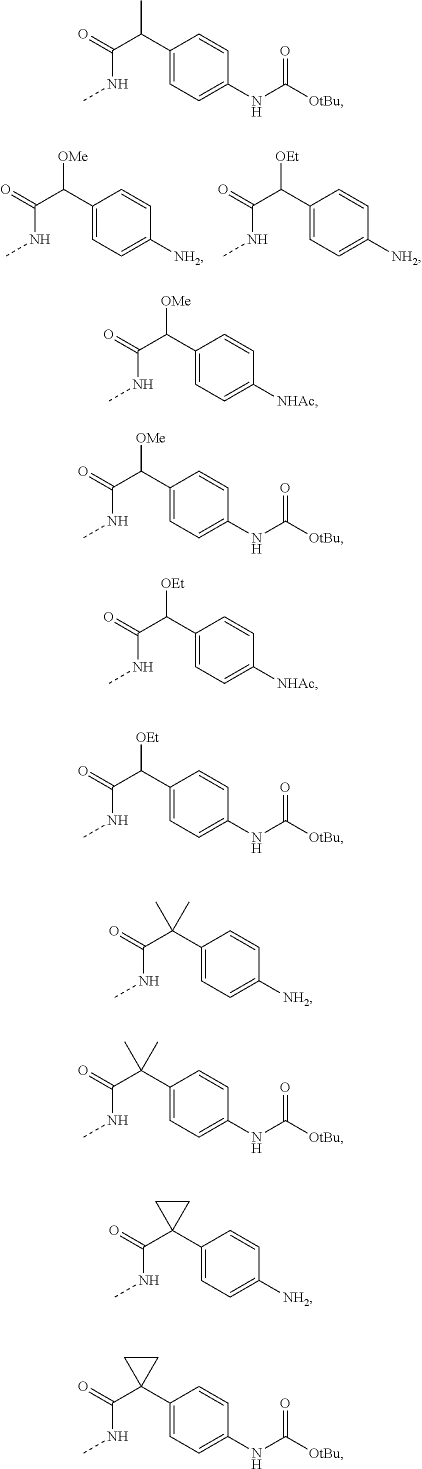

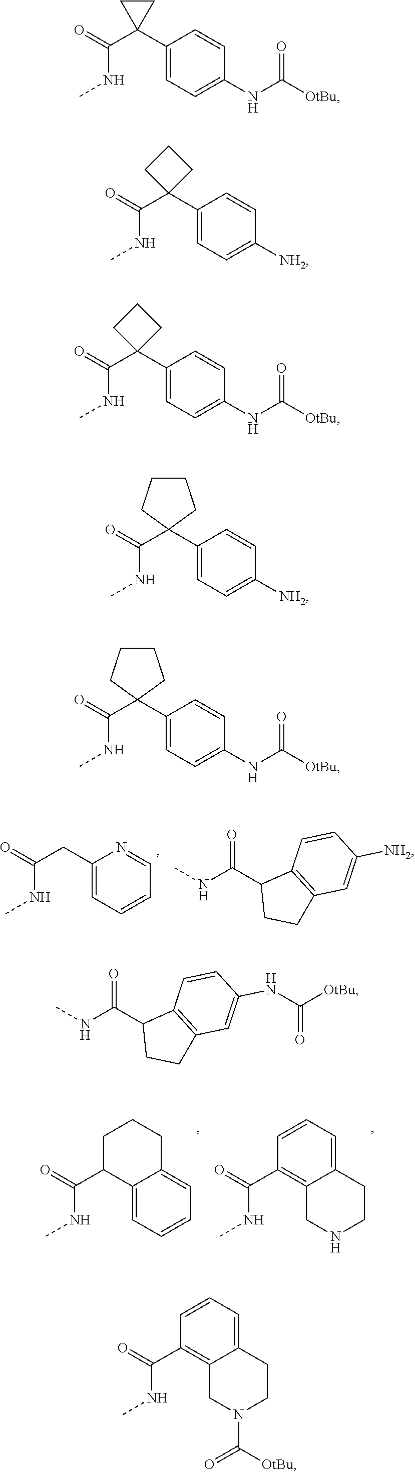

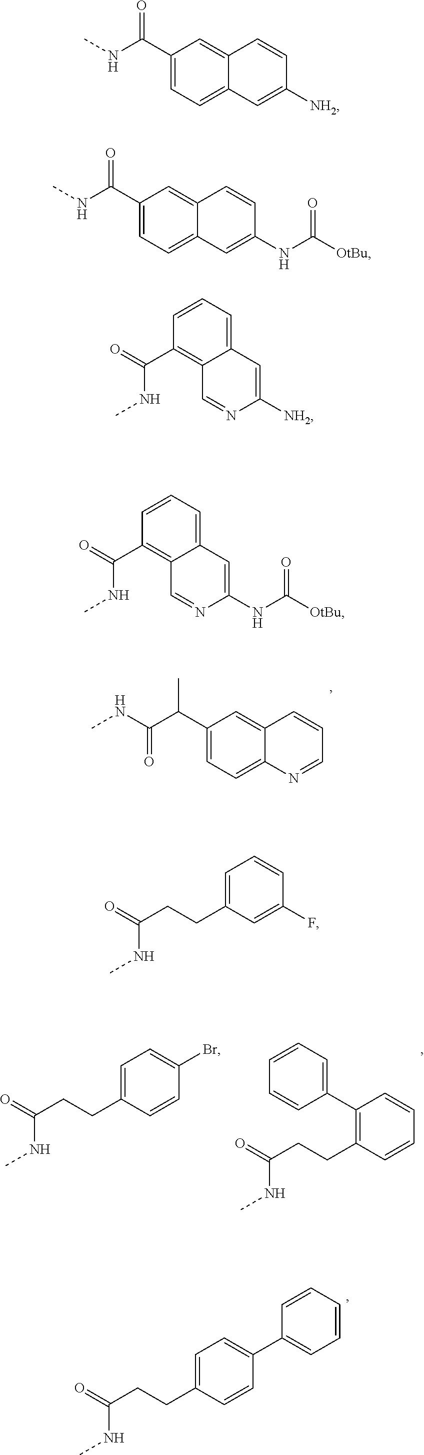

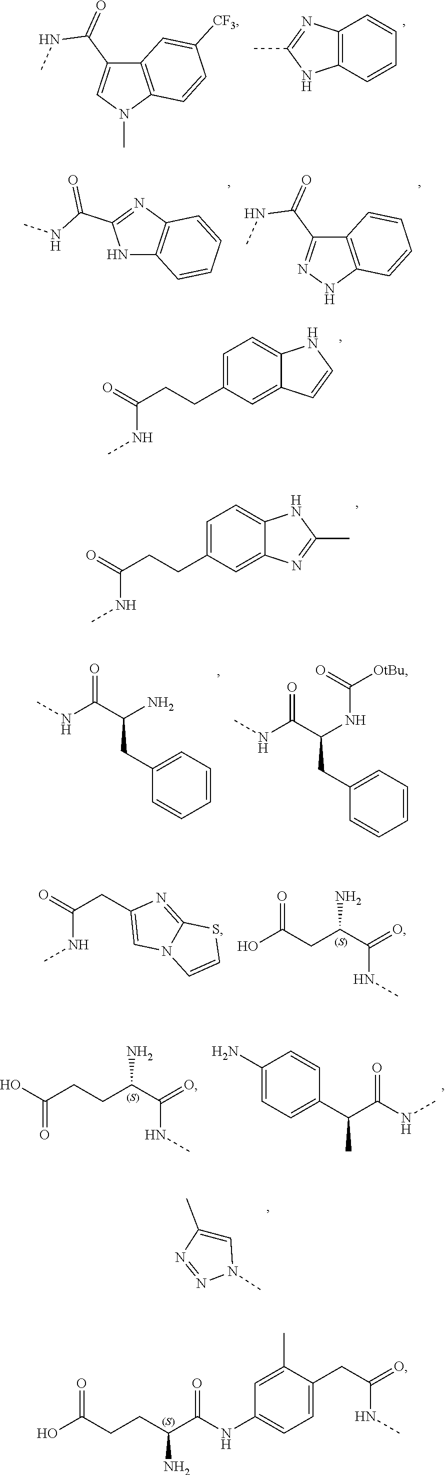

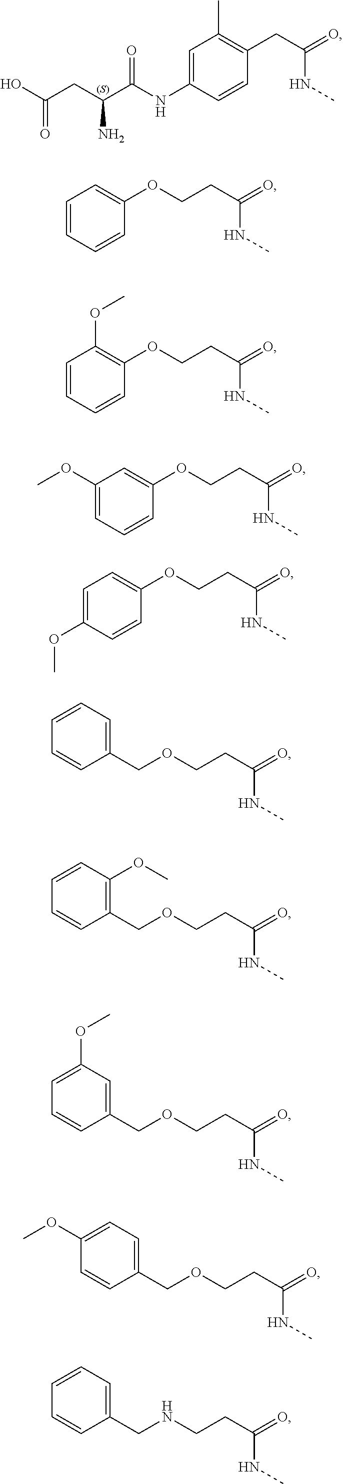

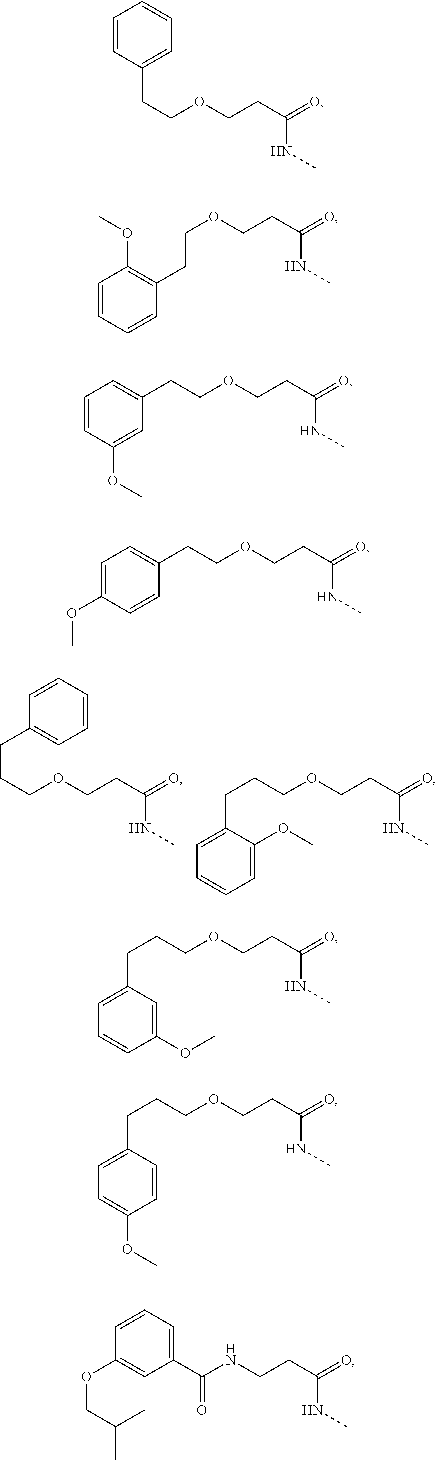

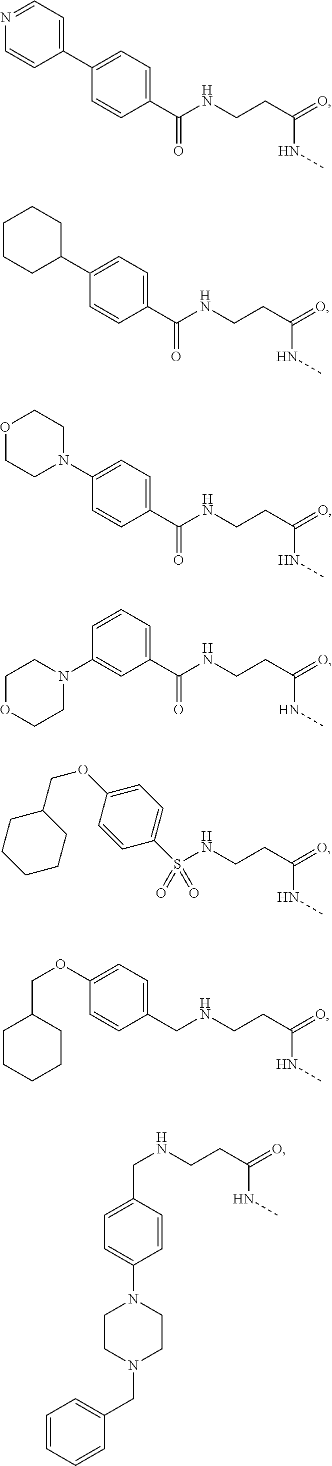

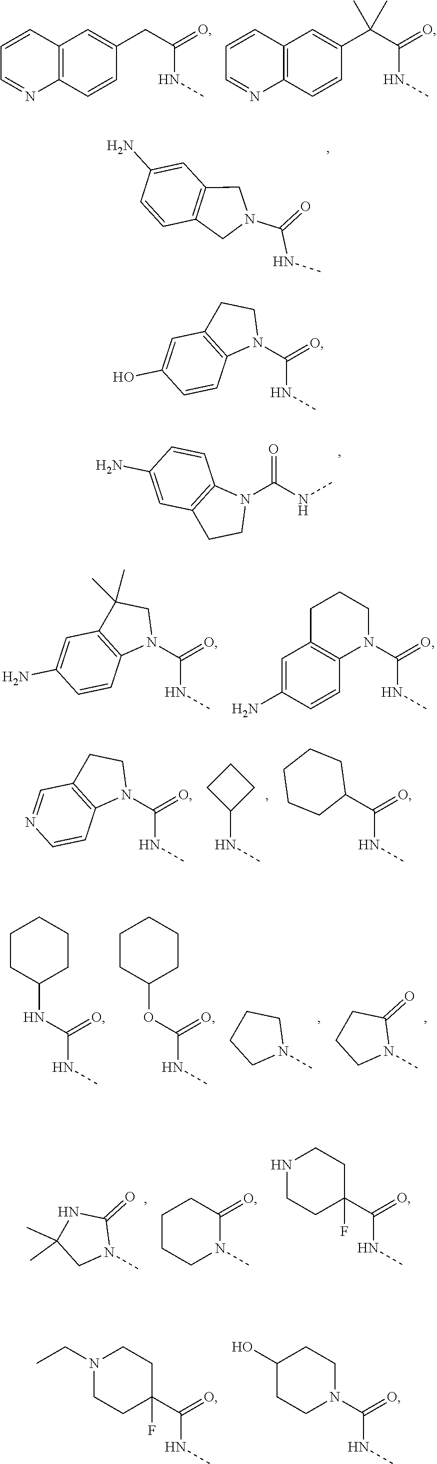

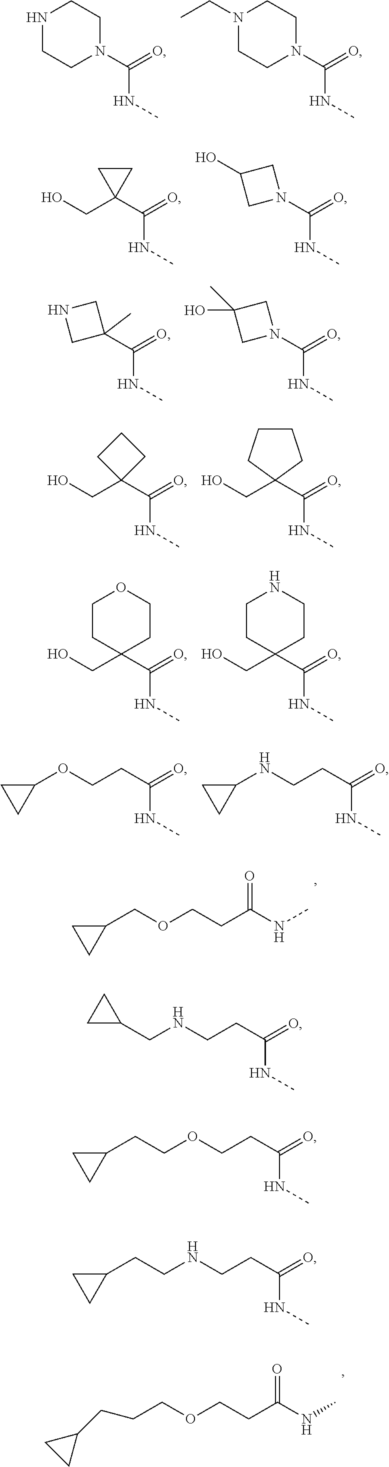

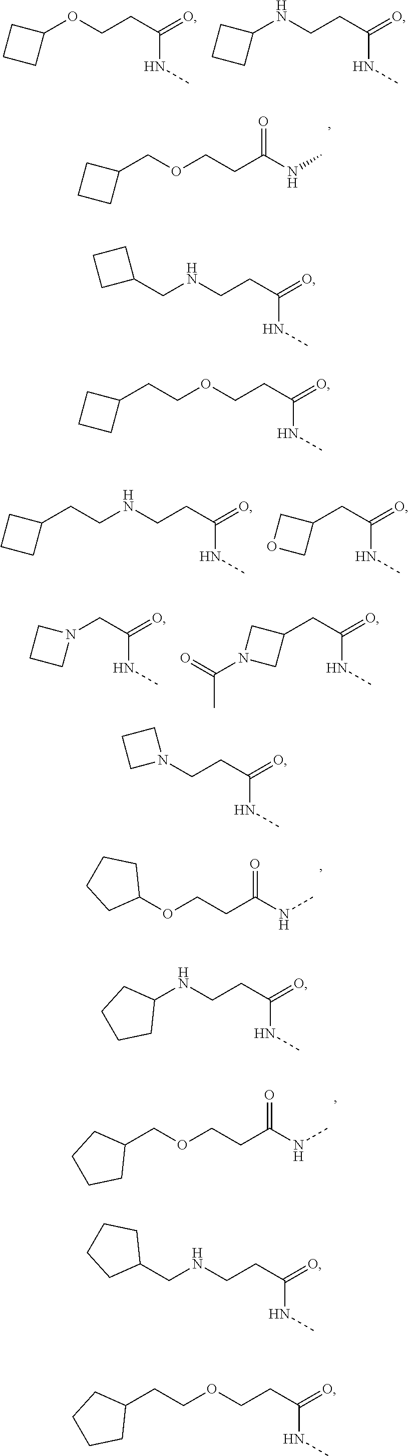

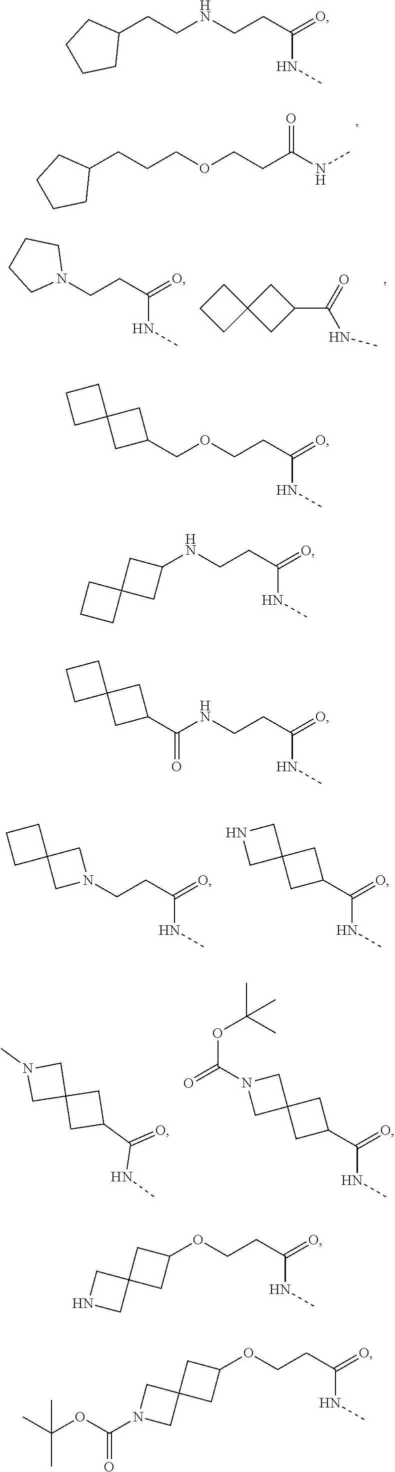

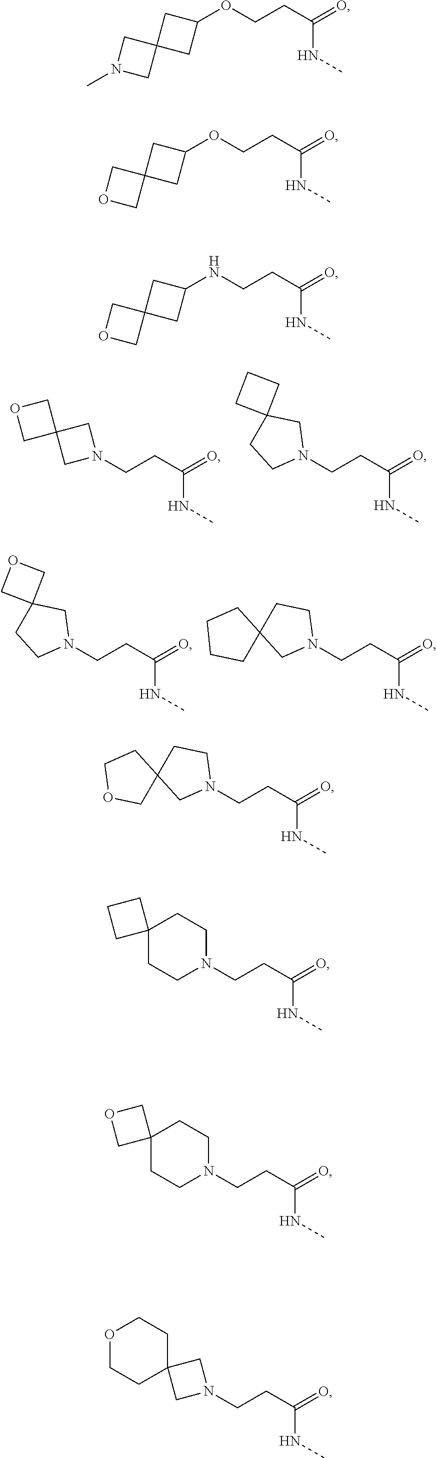

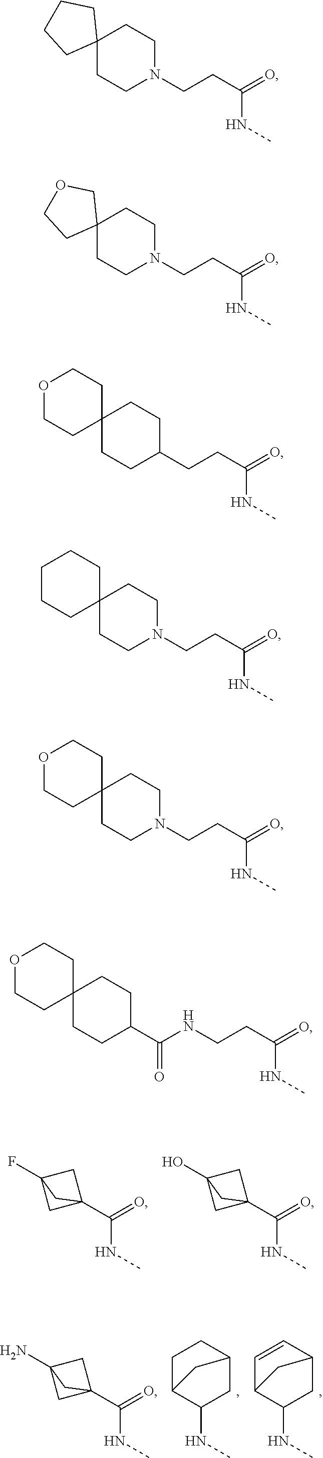

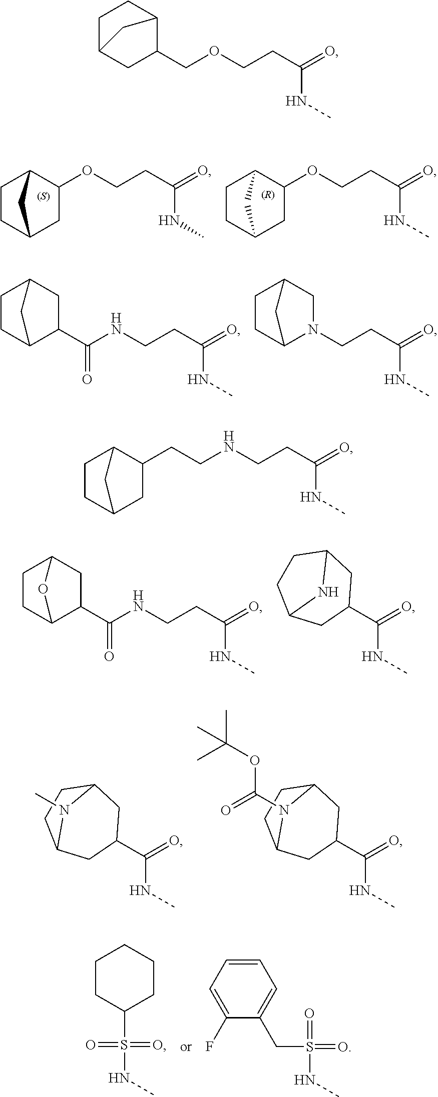

9. The compound according to claim 1, wherein the compound has any one of the formulae (II-1)-(II-16), (III-1)-(III-10), (IV-1)-(IV-10), and (V-1)-(V-9) 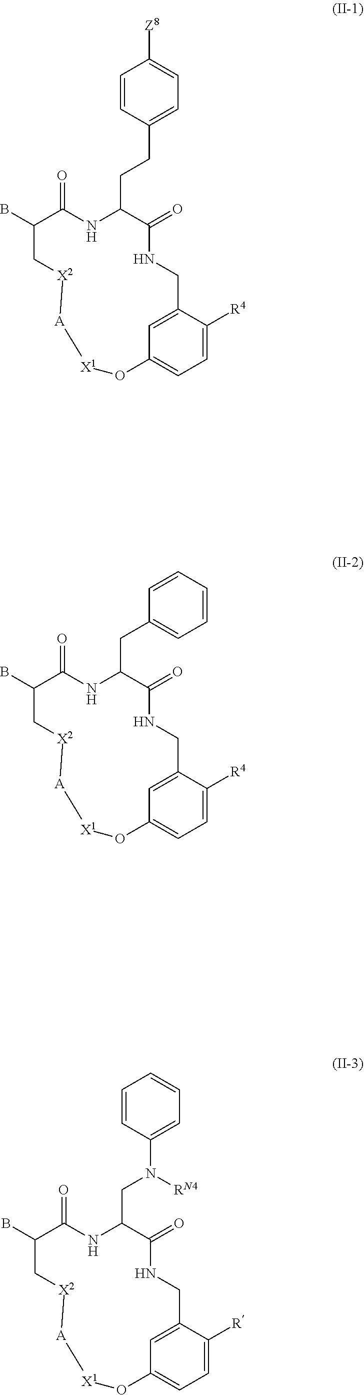 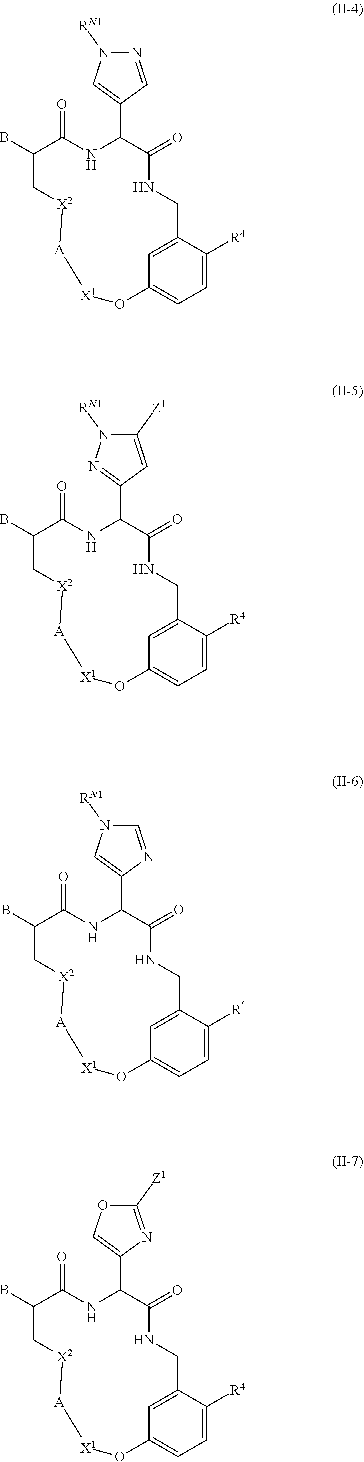 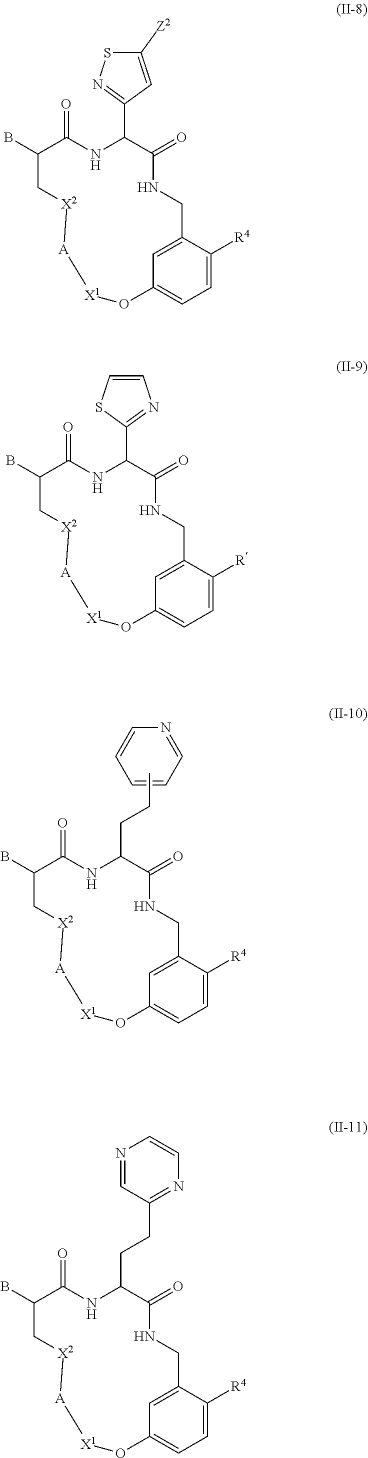 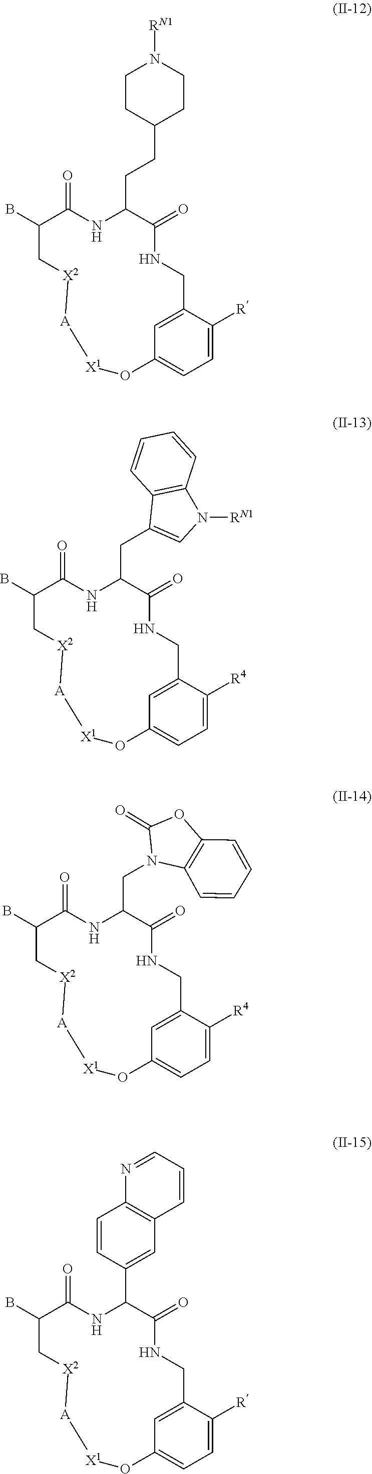 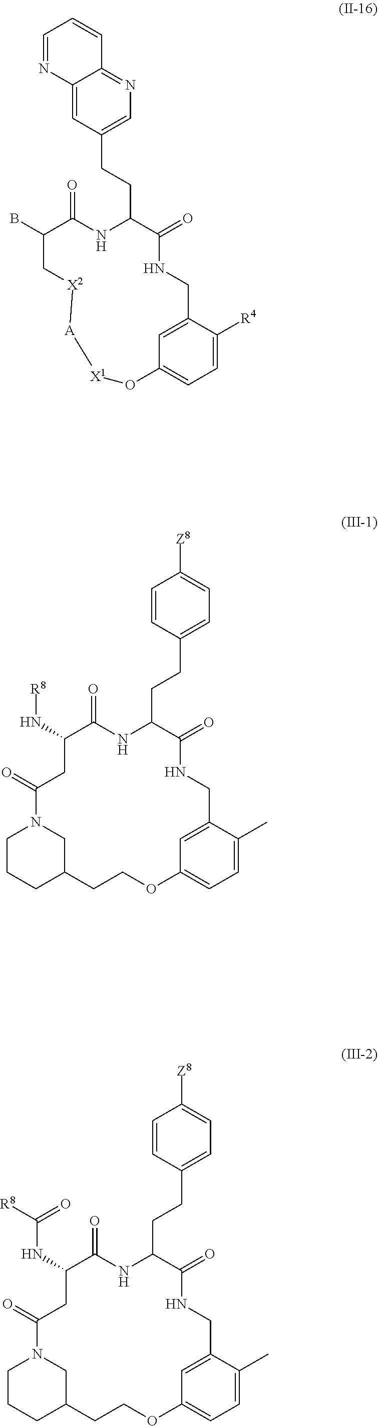 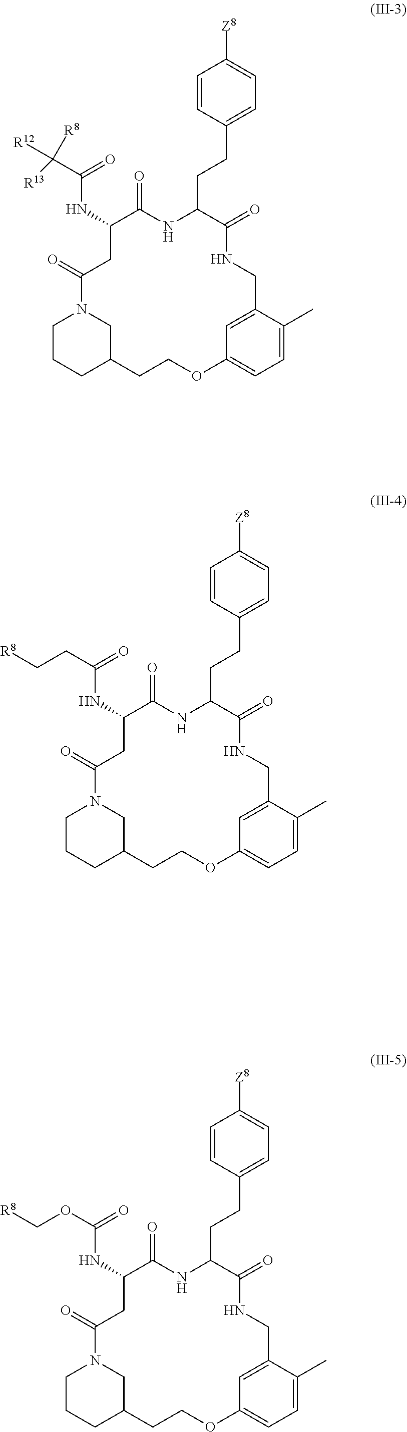 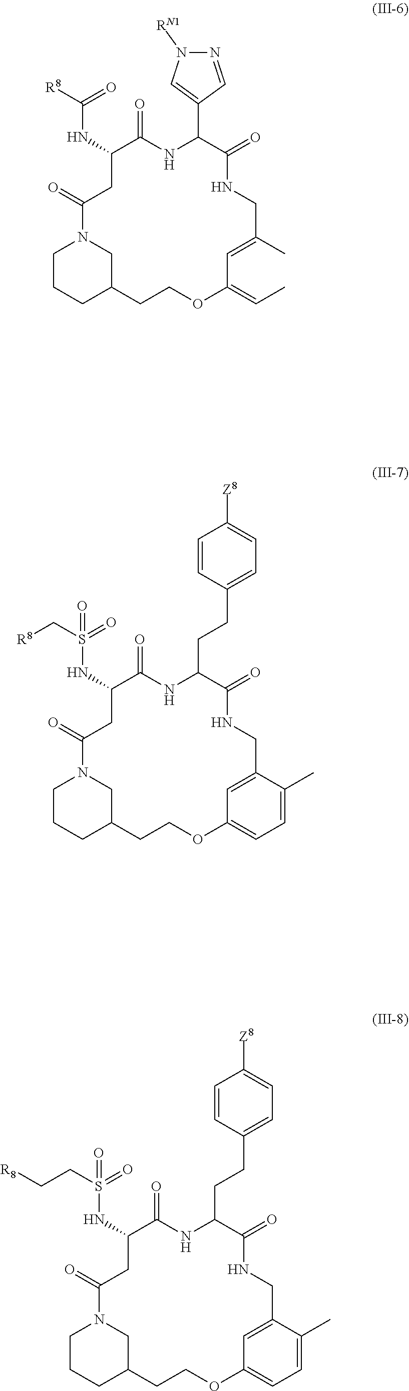 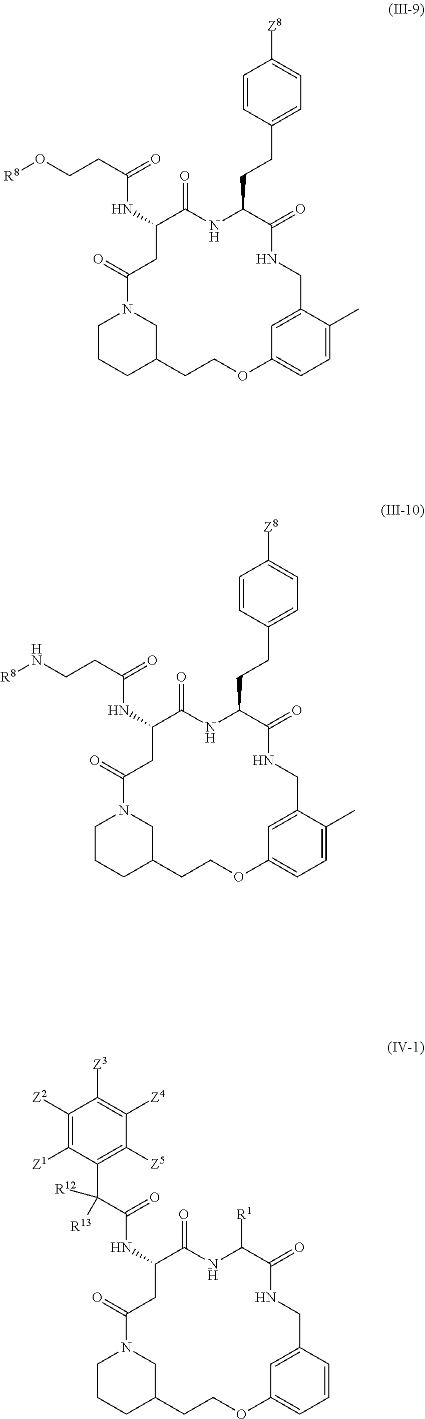 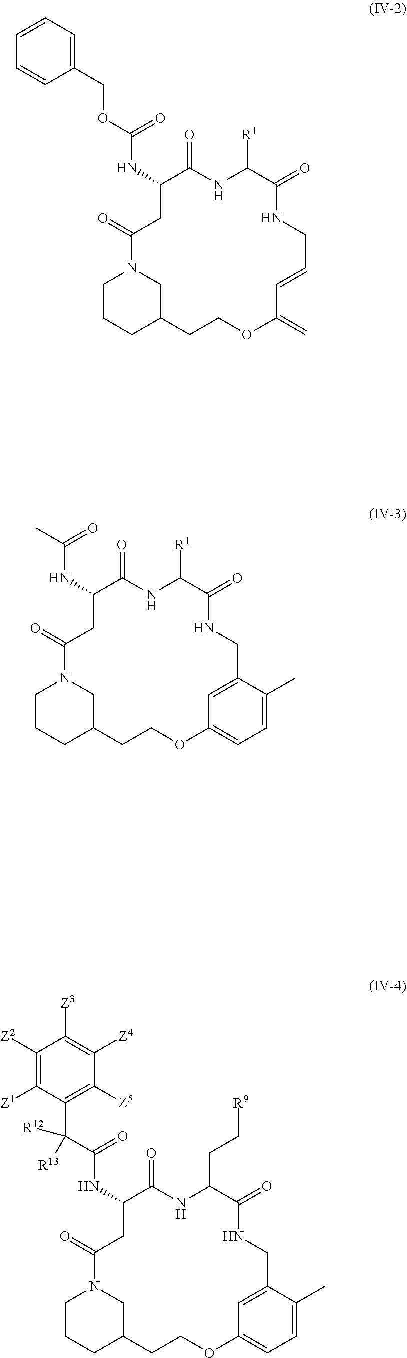 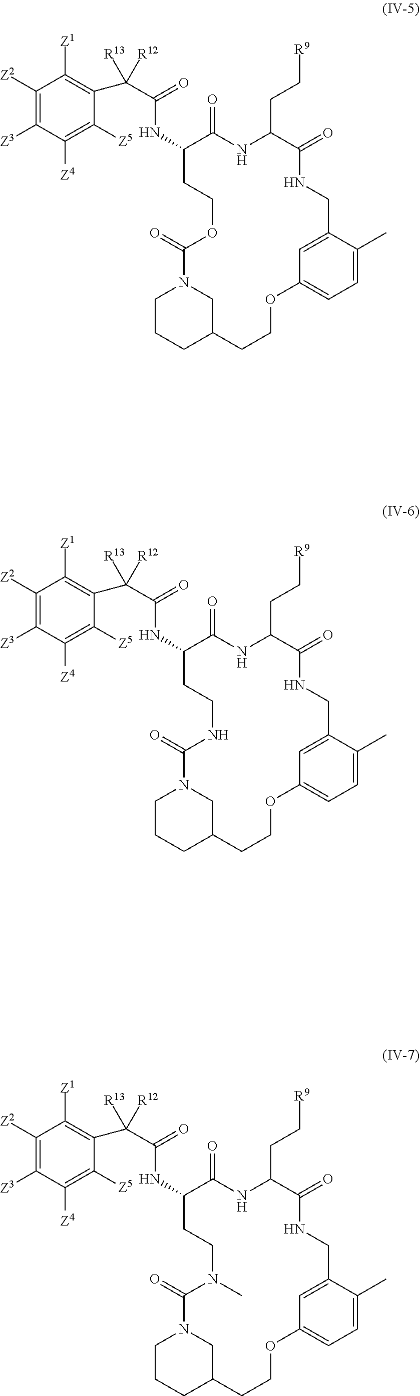 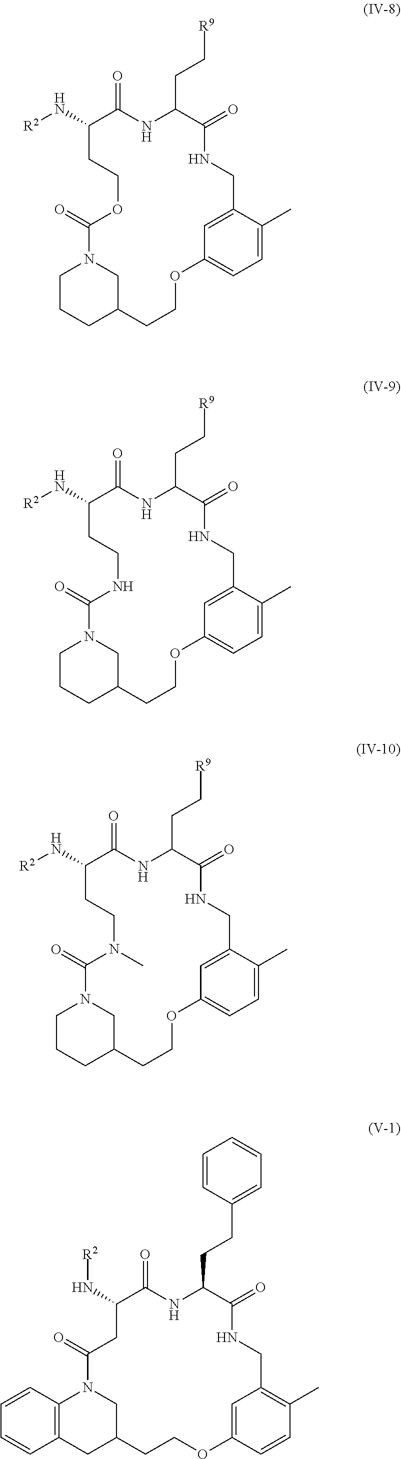 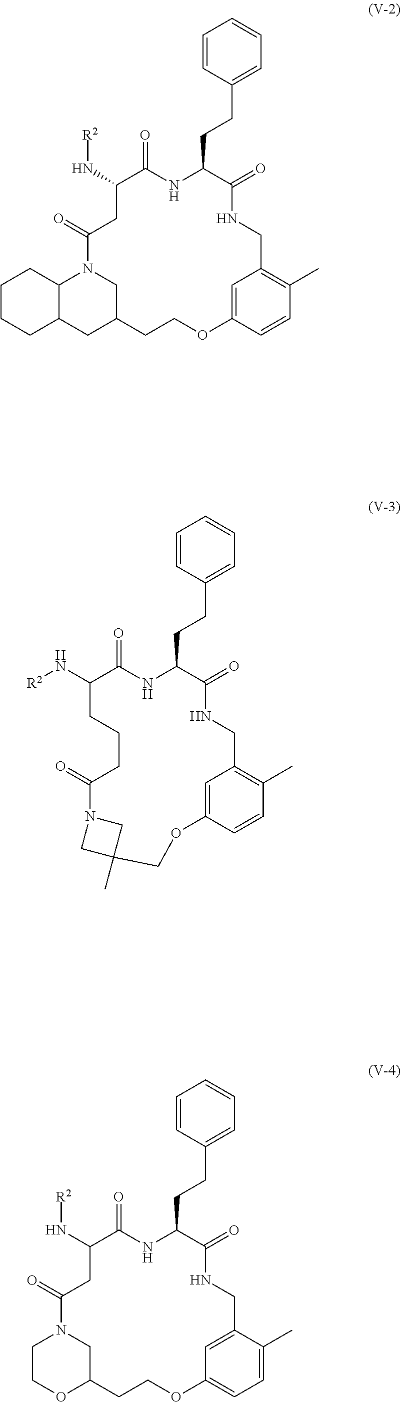 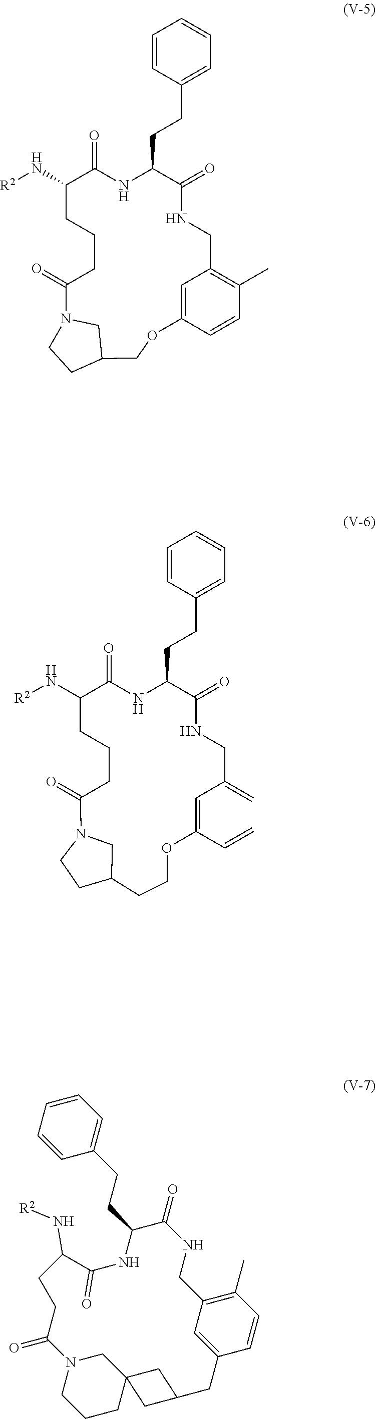 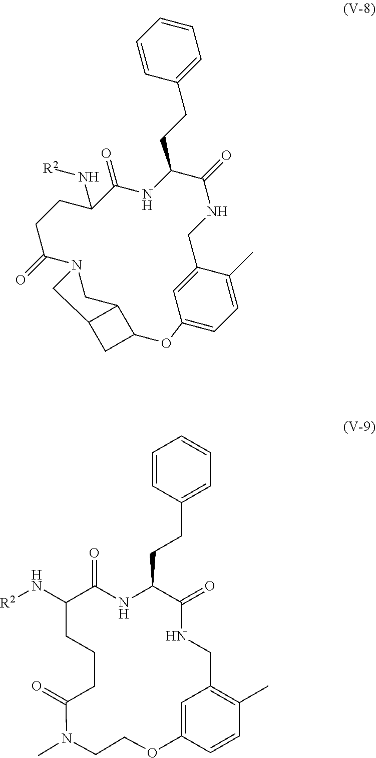
wherein A, B, R¹, R², R⁴, R⁸, R⁹, R¹², R¹³, R^{N1}, R^{N4}, X¹, X², Z¹, Z², Z³, Z⁴, Z⁵ and Z⁸ have the same meanings as defined in claim 1.

10. The compound according to claim 1 selected from the group consisting of:

| Compound | IUPAC Name |
|---|---|
| 7 | benzyl ((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)carbamate |
| 8 | (9S,12S)-12-amino-5⁴-methyl-9-phenethyl-4-oxa-7,10-diaza-1(3,1)- |
| | piperidina-5(1,3)-benzenacyclotetradecaphane-8,11,14-trione |
| 13 | (9S)-5⁴-methyl-9-phenethyl-4-oxa-7,10-diaza-1(3,1)-piperidina-5(1,3)- |
| | benzenacyclotetradecaphane-8,11,14-trione |
| 16 | (9S)-5⁴-methyl-9-phenethyl-4-oxa-7,10-diaza-1(3,1)-piperidina-5(1,3)- |
| | benzenacyclohexadecaphane-8,11,16-trione |
| 19 | (9S)-5⁴-methyl-9-phenethyl-4-oxa-7,10-diaza-1(3,1)-piperidina-5(1,3)- |
| | benzenacyclopentadecaphane-8,11,15-trione |
| 22 | (9S)-5⁴-methyl-9-phenethyl-4-oxa-7,10-diaza-1(3,1)-piperidina-5(1,3)- |
| | benzenacycloheptadecaphane-8,11,17-trione |
| 25 | 3-(3-fluorophenyl)-N-((9S,12S)-5⁴-methyl-8,11,14-trioxo-9-phenethyl-4- |
| | oxa-7,10-diaza-1(3,1)-piperidina-5(1,3)-benzenacyclotetradecaphane-12- |
| | yl)propanamide |
| 26 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)benzamide |
| 27 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)pivalamide |
| 28 | 3,3-dimethyl-N-((9S,12S)-5⁴-methyl-8,11,14-trioxo-9-phenethyl-4-oxa- |
| | 7,10-diaza-1(3,1)-piperidina-5(1,3)-benzenacyclotetradecaphane-12- |
| | yl)butanamide |
| 29 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-2- |
| | phenylacetamide |
| 30 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-2- |
| | phenylacetamide |
| 31 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-1H- |
| | benzo[d]imidazole-2-carboxamide |
| 32 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)pyrimidine-2- |
| | carboxamide |
| 33 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)acetamide |
| 34 | (9S,12S)-5⁴-methyl-9-phenethyl-12-(pyrimidin-2-ylamino)-4-oxa-7,10- |
| | diaza-1(3,1)-piperidina-5(1,3)-benzenacyclotetradecaphane-8,11,14-trione |
| 35 | (9S,12S)-5⁴-methyl-12-(oxetan-3-ylamino)-9-phenethyl-4-oxa-7,10-diaza- |
| | 1(3,1)-piperidina-5(1,3)-benzenacyclotetradecaphane-8,11,14-trione |
| 36 | (9S,12S)-5⁴-methyl-9-phenethyl-12-(pyridin-2-ylamino)-4-oxa-7,10-diaza- |
| | 1(3,1)-piperidina-5(1,3)-benzenacyclotetradecaphane-8,11,14-trione |
| 37 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12- |
| | yl)methanesulfonamide |
| 38 | 1-ethyl-3-((9S,12S)-5⁴-methyl-8,11,14-trioxo-9-phenethyl-4-oxa-7,10- |
| | diaza-1(3,1)-piperidina-5(1,3)-benzenacyclotetradecaphane-12-yl)urea |
| 39 | ethyl ((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)carbamate |
| 40 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)picolinamide |
| 41 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)nicotinamide |
| 42 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12- |
| | yl)isonicotinamide |
| 43 | (9S,12S)-12-(2,5-dioxopyrrolidin-1-yl)-5⁴-methyl-9-phenethyl-4-oxa-7,10- |
| | diaza-1(3,1)-piperidina-5(1,3)-benzenacyclotetradecaphane-8,11,14-trione |
| 44 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-3- |
| | phenylpropane-1-sulfonamide |
| 45 | (9S,12S)-12-((1H-benzo[d]imidazol-2-yl)amino)-5⁴-methyl-9-phenethyl-4- |
| | oxa-7,10-diaza-1(3,1)-piperidina-5(1,3)-benzenacyclotetradecaphane- |
| | 8,11,14-trione |
| 51 | 5⁴-methyl-9-(2-(pyridin-3-yl)ethyl)-4-oxa-7,10-diaza-1(3,1)-piperidina- |
| | 5(1,3)-benzenacyclotetradecaphane-8,11,14-trione |
| 59 | (9R)-⁵⁴,5⁶-dimethyl-9-phenethyl-4-oxa-7,10-diaza-1(3,1)-piperidina-5(1,3)- |
| | benzenacyclotetradecaphane-8,11,14-trione |
| 60 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-1H-indazole- |
| | 3-carboxamide |
| 61 | 2-(3-chlorophenyl)-N-((9S,12S)-5⁴-methyl-8,11,14-trioxo-9-phenethyl-4- |
| | oxa-7,10-diaza-1(3,1)-piperidina-5(1,3)-benzenacyclotetradecaphane-12- |
| | yl)acetamide |
| 62 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-2-(m- |
| | tolyl)acetamide |
| 63 | 2-(3,4-dimethoxyphenyl)-N-((9S,12S)-5⁴-methyl-8,11,14-trioxo-9- |
| | phenethyl-4-oxa-7,10-diaza-1(3,1)-piperidina-5(1,3)- |
| | benzenacyclotetradecaphane-12-yl)acetamide |
| 64 | 2-(3,4-dimethoxyphenyl)-N-((9S,12S)-5⁴-methyl-8,11,14-trioxo-9- |
| | phenethyl-4-oxa-7,10-diaza-1(3,1)-piperidina-5(1,3)- |
| | benzenacyclotetradecaphane-12-yl)butanamide |
| 77 | (9S)-9-((1H-indol-3-yl)methyl)-5⁴-methyl-4-oxa-7,10-diaza-1(3,1)- |
| | piperidina-5(1,3)-benzenacyclotetradecaphane-8,11,14-trione |
| 78 | (9S)-5⁴,9-dimethyl-4-oxa-7,10-diaza-1(3,1)-piperidina-5(1,3)- |
| | benzenacyclotetradecaphane-8,11,14-trione |
| 79 | (9S)-9-benzyl-5⁴-methyl-4-oxa-7,10-diaza-1(3,1)-piperidina-5(1,3)- |
| | benzenacyclotetradecaphane-8,11,14-trione |
| 80 | (2R)-N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-2- |
| | phenylpropanamide |
| 81 | (2S)-N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-2- |
| | phenylpropanamide |
| 82 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-1,2,3,4- |
| | tetrahydronaphthalene-1-carboxamide |
| 83 | 2-(4-methoxyphenyl)-N-((9S,12S)-5⁴-methyl-8,11,14-trioxo-9-phenethyl-4- |
| | oxa-7,10-diaza-1(3,1)-piperidina-5(1,3)-benzenacyclotetradecaphane-12- |
| | yl)acetamide |
| 84 | 2-(2-methoxyphenyl)-N-((9S,12S)-5⁴-methyl-8,11,14-trioxo-9-phenethyl-4- |
| | oxa-7,10-diaza-1(3,1)-piperidina-5(1,3)-benzenacyclotetradecaphane-12- |
| | yl)acetamide |
| 85 | 2-(3-methoxyphenyl)-N-((9S,12S)-5⁴-methyl-8,11,14-trioxo-9-phenethyl-4- |
| | oxa-7,10-diaza-1(3,1)-piperidina-5(1,3)-benzenacyclotetradecaphane-12- |
| | yl)acetamide |
| 86 | (2R)-2-methoxy-N-((9S,12S)-5⁴-methyl-8,11,14-trioxo-9-phenethyl-4-oxa- |
| | 7,10-diaza-1(3,1)-piperidina-5(1,3)-benzenacyclotetradecaphane-12-yl)-2- |
| | phenylacetamide |
| 87 | 1-methyl-N-((9S,12S)-5⁴-methyl-8,11,14-trioxo-9-phenethyl-4-oxa-7,10- |
| | diaza-1(3,1)-piperidina-5(1,3)-benzenacyclotetradecaphane-12-yl)-5- |
| | (trifluoromethyl)-1H-indole-2-carboxamide |
| 88 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-2- |
| | morpholinoacetamide |
| 89 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-2-(pyrrolidin- |
| | 1-yl)acetamide |
| 95 | (9S)-5⁴,5⁵-dimethyl-9-phenethyl-4-oxa-7,10-diaza-1(3,1)-piperidina-5(1,3)- |
| | benzenacyclotetradecaphane-8,11,14-trione (95) |
| 105 | benzyl ((12S)-5⁴-methyl-8,11,14-trioxo-9-(2-(pyrazin-2-yl)ethyl)-4-oxa- |
| | 7,10-diaza-1(3,1)-piperidina-5(1,3)-benzenacyclotetradecaphane-12- |
| | yl)carbamate |
| 106 | (12S)-amino-5⁴-methyl-8,11,14-trioxo-9-(2-(pyrazin-2-yl)ethyl)-4-oxa-7,10- |
| | diaza-1(3,1)-piperidina-5(1,3)-benzenacyclotetradecaphane |
| 107 | (2S)-N-((12S)-5⁴-methyl-8,11,14-trioxo-9-(2-(pyrazin-2-yl)ethyl)-4-oxa- |
| | 7,10-diaza-1(3,1)-piperidina-5(1,3)-benzenacyclotetradecaphane-12-yl)-2- |
| | phenylpropanamide |
| 116 | 5⁴-methyl-9-(1-methyl-1H-pyrazol-4-yl)-4-oxa-7,10-diaza-1(3,1)-piperidina- |
| | 5(1,3)-benzenacyclotetradecaphane-8,11,14-trione |
| 120 | (9S)-9-((1H-indol-3-yl)methyl)-⁵⁴,⁵⁵-dimethyl-4-oxa-7,10-diaza-1(3,1)- |
| | piperidina-5(1,3)-benzenacyclotetradecaphane-8,11,14-trione |
| 121 | tert-butyl 4-(2-(((9S,12S)-5⁴-methyl-8,11,14-trioxo-9-phenethyl-4-oxa-7,10- |
| | diaza-1(3,1)-piperidina-5(1,3)-benzenacyclotetradecaphane-12-yl)amino)- |
| | 2-oxoethyl)piperazine-1-carboxylate |
| 122 | tert-butyl (3-(2-(((9S,12S)-5⁴-methyl-8,11,14-trioxo-9-phenethyl-4-oxa- |
| | 7,10-diaza-1(3,1)-piperidina-5(1,3)-benzenacyclotetradecaphane-12- |
| | yl)amino)-2-oxoethyl)phenyl)carbamate |
| 123 | tert-butyl (4-(2-(((9S,12S)-5⁴-methyl-8,11,14-trioxo-9-phenethyl-4-oxa- |
| | 7,10-diaza-1(3,1)-piperidina-5(1,3)-benzenacyclotetradecaphane-12- |
| | yl)amino)-2-oxoethyl)phenyl)carbamate |
| 124 | tert-butyl methyl(2-(((9S,12S)-5⁴-methyl-8,11,14-trioxo-9-phenethyl-4-oxa- |
| | 7,10-diaza-1(3,1)-piperidina-5(1,3)-benzenacyclotetradecaphane-12- |
| | yl)amino)-2-oxoethyl)carbamate |
| 125 | tert-butyl methyl(2-(2-(((9S,12S)-5⁴-methyl-8,11,14-trioxo-9-phenethyl-4- |
| | oxa-7,10-diaza-1(3,1)-piperidina-5(1,3)-benzenacyclotetradecaphane-12- |
| | yl)amino)-2-oxoethoxy)ethyl)carbamate |
| 126 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-2-(piperazin- |
| | 1-yl)acetamide |
| 127 | 2-(3-aminophenyl)-N-((9S,12S)-5⁴-methyl-8,11,14-trioxo-9-phenethyl-4- |
| | oxa-7,10-diaza-1(3,1)-piperidina-5(1,3)-benzenacyclotetradecaphane-12- |
| | yl)acetamide |
| 128 | 2-(4-aminophenyl)-N-((9S,12S)-5⁴-methyl-8,11,14-trioxo-9-phenethyl-4- |
| | oxa-7,10-diaza-1(3,1)-piperidina-5(1,3)-benzenacyclotetradecaphane-12- |
| | yl)acetamide |
| 129 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-2- |
| | (methylamino)acetamide |
| 130 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-2-(2- |
| | (methylamino)ethoxy)acetamide |
| 131 | tert-butyl 4-fluoro-4-(((9S,12S)-5⁴-methyl-8,11,14-trioxo-9-phenethyl-4- |
| | oxa-7,10-diaza-1(3,1)-piperidina-5(1,3)-benzenacyclotetradecaphane-12- |
| | yl)carbamoyl)piperidine-1-carboxylate |
| 132 | tert-butyl 4,4-difluoro-2-(((9S,12S)-5⁴-methyl-8,11,14-trioxo-9-phenethyl-4- |
| | oxa-7,10-diaza-1(3,1)-piperidina-5(1,3)-benzenacyclotetradecaphane-12- |
| | yl)carbamoyl)pyrrolidine-1-carboxylate |
| 133 | tert-butyl 4-methyl-4-(((9S,12S)-5⁴-methyl-8,11,14-trioxo-9-phenethyl-4- |
| | oxa-7,10-diaza-1(3,1)-piperidina-5(1,3)-benzenacyclotetradecaphane-12- |
| | yl)carbamoyl)piperidine-1-carboxylate |
| 134 | 4-fluoro-N-((9S,12S)-5⁴-methyl-8,11,14-trioxo-9-phenethyl-4-oxa-7,10- |
| | diaza-1(3,1)-piperidina-5(1,3)-benzenacyclotetradecaphane-12- |
| | yl)piperidine-4-carboxamide |
| 135 | (2S)-4,4-difluoro-N-((9S,12S)-5⁴-methyl-8,11,14-trioxo-9-phenethyl-4-oxa- |
| | 7,10-diaza-1(3,1)-piperidina-5(1,3)-benzenacyclotetradecaphane-12- |
| | yl)pyrrolidine-2-carboxamide |
| 136 | 4-methyl-N-((9S,12S)-5⁴-methyl-8,11,14-trioxo-9-phenethyl-4-oxa-7,10- |
| | diaza-1(3,1)-piperidina-5(1,3)-benzenacyclotetradecaphane-12- |
| | yl)piperidine-4-carboxamide |
| 143 | N-((9S,12S)-5⁴-methyl-8,11,15-trioxo-9-phenethyl-4-oxa-7,10-diaza- |
| | 1(2,4)-morpholina-5(1,3)-benzenacyclopentadecaphane-12-yl)acetamide |
| 148 | N-((7S,10S)-3⁴-methyl-6,9,13-trioxo-7-phenethyl-2-oxa-5,8-diaza-1(3,1)- |
| | piperidina-3(1,3)-benzenacyclotridecaphane-10-yl)acetamide |
| 149 | tert-butyl 4-ethyl-4-(((9S,12S)-5⁴-methyl-8,11,14-trioxo-9-phenethyl-4-oxa- |
| | 7,10-diaza-1(3,1)-piperidina-5(1,3)-benzenacyclotetradecaphane-12- |
| | yl)carbamoyl)piperidine-1-carboxylate |
| 150 | tert-butyl 3,3-difluoro-4-(((9S,12S)-5⁴-methyl-8,11,14-trioxo-9-phenethyl-4- |
| | oxa-7,10-diaza-1(3,1)-piperidina-5(1,3)-benzenacyclotetradecaphane-12- |
| | yl)carbamoyl)piperidine-1-carboxylate |
| 151 | 4-ethyl-N-((9S,12S)-5⁴-methyl-8,11,14-trioxo-9-phenethyl-4-oxa-7,10- |
| | diaza-1(3,1)-piperidina-5(1,3)-benzenacyclotetradecaphane-12- |
| | yl)piperidine-4-carboxamide |
| 152 | 3,3-difluoro-N-((9S,12S)-5⁴-methyl-8,11,14-trioxo-9-phenethyl-4-oxa-7,10- |
| | diaza-1(3,1)-piperidina-5(1,3)-benzenacyclotetradecaphane-12- |
| | yl)piperidine-4-carboxamide |
| 153 | tert-butyl 2-(((9S,12S)-5⁴-methyl-8,11,14-trioxo-9-phenethyl-4-oxa-7,10- |
| | diaza-1(3,1)-piperidina-5(1,3)-benzenacyclotetradecaphane-12- |
| | yl)carbamoyl)-5-(trifluoromethyl)pyrrolidine-1-carboxylate |
| 154 | tert-butyl 2-(((9S,12S)-5⁴-methyl-8,11,14-trioxo-9-phenethyl-4-oxa-7,10- |
| | diaza-1(3,1)-piperidina-5(1,3)-benzenacyclotetradecaphane-12- |
| | yl)carbamoyl)piperidine-1-carboxylate |
| 155 | tert-butyl (3-(((9S,12S)-5⁴-methyl-8,11,14-trioxo-9-phenethyl-4-oxa-7,10- |
| | diaza-1(3,1)-piperidina-5(1,3)-benzenacyclotetradecaphane-12- |
| | yl)carbamoyl)oxetan-3-yl)carbamate |
| 156 | tert-butyl (3-(((9S,12S)-5⁴-methyl-8,11,14-trioxo-9-phenethyl-4-oxa-7,10- |
| | diaza-1(3,1)-piperidina-5(1,3)-benzenacyclotetradecaphane-12- |
| | yl)carbamoyl)tetrahydrofuran-3-yl)carbamate |
| 157 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-5- |
| | (trifluoromethyl)pyrrolidine-2-carboxamide |
| 158 | 3-amino-N-((9S,12S)-5⁴-methyl-8,11,14-trioxo-9-phenethyl-4-oxa-7,10- |
| | diaza-1(3,1)-piperidina-5(1,3)-benzenacyclotetradecaphane-12- |
| | yl)oxetane-3-carboxamide |
| 159 | 3-amino-N-((9S,12S)-5⁴-methyl-8,11,14-trioxo-9-phenethyl-4-oxa-7,10- |
| | diaza-1(3,1)-piperidina-5(1,3)-benzenacyclotetradecaphane-12- |
| | yl)tetrahydrofuran-3-carboxamide |
| 160 | (2R)-N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)azetidine-2- |
| | carboxamide |
| 161 | tert-butyl 2-(((9S,12S)-5⁴-methyl-8,11,14-trioxo-9-phenethyl-4-oxa-7,10- |
| | diaza-1(3,1)-piperidina-5(1,3)-benzenacyclotetradecaphane-12- |
| | yl)carbamoyl)azetidine-1-carboxylate |
| 168 | N-((10S,13S)-1⁴-methyl-8,11,14-trioxo-13-phenethyl-2-oxa-7,12,15-triaza- |
| | 1(1,3)-benzenacyclohexadecaphane-10-yl)acetamide |
| 171 | N-((11S,14S)-1⁴-methyl-8,12,15-trioxo-14-phenethyl-2-oxa-7,13,16-triaza- |
| | 1(1,3)-benzenacycloheptadecaphane-11-yl)acetamide |
| 172 | 2-(4-aminophenyl)-N-((13R,9S,12S)-5⁴-methyl-8,11,14-trioxo-9-phenethyl- |
| | 4-oxa-7,10-diaza-1(3,1)-piperidina-5(1,3)-benzenacyclotetradecaphane- |
| | 12-yl)acetamide |
| 173 | 2-(4-aminophenyl)-N-((13S,9S,12S)-5⁴-methyl-8,11,14-trioxo-9-phenethyl- |
| | 4-oxa-7,10-diaza-1(3,1)-piperidina-5(1,3)-benzenacyclotetradecaphane- |
| | 12-yl)acetamide |
| 174 | 3-(2-methyl-1H-benzo[d]imidazol-6-yl)-N-((9S,12S)-5⁴-methyl-8,11,14- |
| | trioxo-9-phenethyl-4-oxa-7,10-diaza-1(3,1)-piperidina-5(1,3)- |
| | benzenacyclotetradecaphane-12-yl)propanamide |
| 175 | 3-(4-bromophenyl)-N-((9S,12S)-5⁴-methyl-8,11,14-trioxo-9-phenethyl-4- |
| | oxa-7,10-diaza-1(3,1)-piperidina-5(1,3)-benzenacyclotetradecaphane-12- |
| | yl)propanamide |
| 176 | 3-([1,1′-biphenyl]-4-yl)-N-((9S,12S)-5⁴-methyl-8,11,14-trioxo-9-phenethyl- |
| | 4-oxa-7,10-diaza-1(3,1)-piperidina-5(1,3)-benzenacyclotetradecaphane- |
| | 12-yl)propanamide |
| 177 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-3-(2- |
| | phenoxyphenyl)propanamide |
| 178 | tert-butyl ((2S)-1-(((9S,12S)-5⁴-methyl-8,11,14-trioxo-9-phenethyl-4-oxa- |
| | 7,10-diaza-1(3,1)-piperidina-5(1,3)-benzenacyclotetradecaphane-12- |
| | yl)amino)-1-oxo-3-phenylpropan-2-yl)carbamate |
| 179 | (2R)-2-amino-N-((9S,12S)-5⁴-methyl-8,11,14-trioxo-9-phenethyl-4-oxa- |
| | 7,10-diaza-1(3,1)-piperidina-5(1,3)-benzenacyclotetradecaphane-12-yl)-3- |
| | phenylpropanamide |
| 180 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12- |
| | yl)ethenesulfonamide |
| 181 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)acrylamide |
| 182 | 3-(1H-indol-5-yl)-N-((9S,12S)-5⁴-methyl-8,11,14-trioxo-9-phenethyl-4-oxa- |
| | 7,10-diaza-1(3,1)-piperidina-5(1,3)-benzenacyclotetradecaphane-12- |
| | yl)propanamide |
| 186 | N-((9S,12S)-5⁴-methyl-8,11,16-trioxo-9-phenethyl-4-oxa-7,10-diaza- |
| | 1(2,4)-morpholina-5(1,3)-benzenacyclohexadecaphane-12-yl)acetamide |
| 191 | N-((11R,15S,7S,10S)-34-methyl-6,9,14-trioxo-7-phenethyl-2-oxa-13,5,8- |
| | triaza-1(7,3)-bicyclo[3.2.0]heptana-3(1,3)-benzenacyclotetradecaphane- |
| | 10-yl)acetamide |
| 195 | benzyl ((9S,12S)-5⁴-methyl-8,11,16-trioxo-9-phenethyl-4-oxa-7,10-diaza- |
| | 1(3,1)-piperidina-5(1,3)-benzenacyclohexadecaphane-12-yl)carbamate |
| 199 | benzyl ((9S,12S)-5⁴-methyl-8,11,15-trioxo-9-phenethyl-4-oxa-7,10-diaza- |
| | 1(3,1)-piperidina-5(1,3)-benzenacyclopentadecaphane-12-yl)carbamate |
| 205 | N-((9S,12S)-5⁴-fluoro-8,11,14-trioxo-9-phenethyl-4-oxa-7,10-diaza-1(3,1)- |
| | piperidina-5(1,3)-benzenacyclotetradecaphane-12-yl)acetamide |
| 211 | N-((9S,12S)-5⁴-methoxy-8,11,14-trioxo-9-phenethyl-4-oxa-7,10-diaza- |
| | 1(3,1)-piperidina-5(1,3)-benzenacyclotetradecaphane-12-yl)acetamide |
| 212 | (9S,12S)-5⁴-methyl-12-amino-8,11,16-trioxo-9-phenethyl-4-oxa-7,10- |
| | diaza-1(3,1)-piperidina-5(1,3)-benzenacyclohexadecaphane |
| 213 | N-((9S,12S)-5⁴-methyl-8,11,16-trioxo-9-phenethyl-4-oxa-7,10-diaza- |
| | 1(3,1)-piperidina-5(1,3)-benzenacyclohexadecaphane-12-yl)acetamide |
| 214 | (9S,12S)-12-amino-5⁴-methyl-8,11,15-trioxo-9-phenethyl-4-oxa-7,10- |
| | diaza-1(3,1)-piperidina-5(1,3)-benzenacyclopentadecaphane) |
| 215 | N-((9S,12S)-5⁴-methyl-8,11,15-trioxo-9-phenethyl-4-oxa-7,10-diaza- |
| | 1(3,1)-piperidina-5(1,3)-benzenacyclopentadecaphane-12-yl)acetamide |
| 216 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-1- |
| | phenylmethanesulfonamide |
| 217 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-2- |
| | phenylethane-1-sulfonamide |
| 218 | 2,5,8,11,14,17,20-heptaoxadocosan-22-yl ((9R,12R)-5⁴-methyl-8,11,14- |
| | trioxo-9-phenethyl-4-oxa-7,10-diaza-1(3,1)-piperidina-5(1,3)- |
| | benzenacyclotetradecaphane-12-yl)carbamate |
| 219 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-2,5,8,11,14- |
| | pentaoxaheptadecan-17-amide |
| 220 | ((9R,12R)-5⁴-methyl-8,11,14-trioxo-9-phenethyl-4-oxa-7,10-diaza-1(3,1)- |
| | piperidina-5(1,3)-benzenacyclotetradecaphane-12-yl)carbamate |
| 252 | N-((8S,11S)-1⁴-methyl-6,9,12-trioxo-11-phenethyl-2-oxa-5,10,13-triaza- |
| | 1(1,3)-benzenacyclotetradecaphane-8-yl)acetamide |
| 253 | N-((9S,12S)-1⁴-methyl-7,10,13-trioxo-12-phenethyl-2-oxa-6,11,14-triaza- |
| | 1(1,3)-benzenacyclopentadecaphane-9-yl)acetamide |
| 254 | N-((7S,10S)-3⁴-methyl-6,9,12-trioxo-7-phenethyl-2-oxa-5,8-diaza-1(4,1)- |
| | piperidina-3(1,3)-benzenacyclododecaphane-10-yl)acetamide |
| 255 | N-((7S,10S)-3⁴-methyl-6,9,12-trioxo-7-phenethyl-2-oxa-5,8-diaza-1(3,1)- |
| | piperidina-3(1,3)-benzenacyclododecaphane-10-yl)acetamide |
| 256 | N-((9S,12S)-14,6-dimethyl-7,10,13-trioxo-12-phenethyl-2-oxa-6,11,14- |
| | triaza-1(1,3)-benzenacyclopentadecaphane-9-yl)acetamide |
| 257 | N-((8S,11S)-4⁴-methyl-7,10,13-trioxo-8-phenethyl-3-oxa-6,9-diaza-1(3,1)- |
| | piperidina-4(1,3)-benzenacyclotridecaphane-11-yl)acetamide |
| 258 | N-((9S,12S)-5⁴-methyl-8,11,14-trioxo-9-phenethyl-4-oxa-7,10-diaza- |
| | 1(2,4)-morpholina-5(1,3)-benzenacyclotetradecaphane-12-yl)acetamide |
| 259 | N-((8S,11S)-4⁴-methyl-7,10,13-trioxo-8-phenethyl-3-oxa-6,9-diaza-1(3,1)- |
| | azetidina-4(1,3)-benzenacyclotridecaphane-11-yl)acetamide |
| 260 | N-((8S,11S)-1²,4⁴-dimethyl-7,10,13-trioxo-8-phenethyl-3-oxa-6,9-diaza- |
| | 1(2,1)-azetidina-4(1,3)-benzenacyclotridecaphane-11-yl)acetamide |
| 261 | N-((7S,10S)-3⁴-methyl-6,9,12-trioxo-7-phenethyl-2-oxa-5,8-diaza-1(3,1)- |
| | pyrrolidina-3(1,3)-benzenacyclododecaphane-10-yl)acetamide |
| 265 | N-((8S,11S)-1²,4⁴-dimethyl-7,10,14-trioxo-8-phenethyl-3-oxa-6,9-diaza- |
| | 1(2,1)-azetidina-4(1,3)-benzenacyclotetradecaphane-11-yl)acetamide |
| 269 | N-((8S,11S)-1²,4⁴-dimethyl-7,10,14-trioxo-8-phenethyl-3-oxa-6,9-diaza- |
| | 1(2,1)-azetidina-4(1,3)-benzenacyclotetradecaphane-11-yl)acetamide |
| 280 | N-((11S,14S)-1⁴,6-dimethyl-7,12,15-trioxo-14-phenethyl-2-oxa-6,13,16- |
| | triaza-1(1,3)-benzenacycloheptadecaphane-11-yl)acetamide |
| 281 | N-((8S,11S)-1²,⁴⁴-dimethyl-7,10,15-trioxo-8-phenethyl-3-oxa-6,9-diaza- |
| | 1(2,1)-azetidina-4(1,3)-benzenacyclopentadecaphane-11-yl)acetamide |
| 282 | N-((11S,14S)-1⁴,7-dimethyl-8,12,15-trioxo-14-phenethyl-2-oxa-7,13,16- |
| | triaza-1(1,3)-benzenacycloheptadecaphane-11-yl)acetamide |
| 283 | benzyl ((7S,10S)-3⁴-methyl-6,9,14-trioxo-7-phenethyl-2-oxa-5,8-diaza- |
| | 15(3,1)-piperidina-3(1,3)-benzena-1(1,3)-cyclobutanapentadecaphane-10- |
| | yl)carbamate |
| 284 | 3-hydroxy-2,2-dimethyl-N-((9S,12S)-5⁴-methyl-8,11,14-trioxo-9-phenethyl- |
| | 4-oxa-7,10-diaza-1(3,1)-piperidina-5(1,3)-benzenacyclotetradecaphane- |
| | 12-yl)propanamide |
| 285 | 2-ethyl-2-(hydroxymethyl)-N-((9S,12S)-5⁴-methyl-8,11,14-trioxo-9- |
| | phenethyl-4-oxa-7,10-diaza-1(3,1)-piperidina-5(1,3)- |
| | benzenacyclotetradecaphane-12-yl)butanamide |
| 286 | 3-hydroxy-2-(hydroxymethyl)-2-methyl-N-((9S,12S)-5⁴-methyl-8,11,14- |
| | trioxo-9-phenethyl-4-oxa-7,10-diaza-1(3,1)-piperidina-5(1,3)- |
| | benzenacyclotetradecaphane-12-yl)propanamide |
| 287 | 3-methyl-N-((9S,12S)-5⁴-methyl-8,11,14-trioxo-9-phenethyl-4-oxa-7,10- |
| | diaza-1(3,1)-piperidina-5(1,3)-benzenacyclotetradecaphane-12- |
| | yl)oxetane-3-carboxamide |
| 288 | 4-methyl-N-((9S,12S)-5⁴-methyl-8,11,14-trioxo-9-phenethyl-4-oxa-7,10- |
| | diaza-1(3,1)-piperidina-5(1,3)-benzenacyclotetradecaphane-12- |
| | yl)tetrahydro-2H-pyran-4-carboxamide |
| 289 | benzyl ((13R,9R,12S)-5⁴-methyl-8,11,14-trioxo-9-phenethyl-4-oxa-7,10- |
| | diaza-1(3,1)-piperidina-5(1,3)-benzenacyclotetradecaphane-12- |
| | yl)carbamate |
| 290 | benzyl ((13R,9R,12R)-5⁴-methyl-8,11,14-trioxo-9-phenethyl-4-oxa-7,10- |
| | diaza-1(3,1)-piperidina-5(1,3)-benzenacyclotetradecaphane-12- |
| | yl)carbamate |
| 291 | benzyl ((13R,9S,12R)-5⁴-methyl-8,11,14-trioxo-9-phenethyl-4-oxa-7,10- |
| | diaza-1(3,1)-piperidina-5(1,3)-benzenacyclotetradecaphane-12- |
| | yl)carbamate |
| 292 | benzyl ((13R,9S,12S)-5⁴-methyl-8,11,14-trioxo-9-phenethyl-4-oxa-7,10- |
| | diaza-1(3,1)-piperidina-5(1,3)-benzenacyclotetradecaphane-12- |
| | yl)carbamate |
| 293 | (3-methyloxetan-3-yl)methyl ((9S,12S)-5⁴-methyl-8,11,14-trioxo-9- |
| | phenethyl-4-oxa-7,10-diaza-1(3,1)-piperidina-5(1,3)- |
| | benzenacyclotetradecaphane-12-yl)carbamate |
| 294 | 2-cyano-2-methyl-N-((9S,12S)-5⁴-methyl-8,11,14-trioxo-9-phenethyl-4- |
| | oxa-7,10-diaza-1(3,1)-piperidina-5(1,3)-benzenacyclotetradecaphane-12- |
| | yl)propanamide |
| 295 | N-(1⁴,7-dimethyl-8,13,16-trioxo-15-phenethyl-2-oxa-7,14,17-triaza-1(1,3)- |
| | benzenacyclooctadecaphane-12-yl)acetamide |
| 296 | N-(1⁴,7-dimethyl-8,11,14-trioxo-13-phenethyl-2-oxa-7,12,15-triaza-1(1,3)- |
| | benzenacyclohexadecaphane-10-yl)acetamide |
| 298 | N-(³⁴-methyl-6,9,12-trioxo-7-phenethyl-2-oxa-13,5,8-triaza-1(6,3)- |
| | bicyclo[3.2.0]heptana-3(1,3)-benzenacyclododecaphane-10-yl)acetamide |
| 299 | N-(3⁴-methyl-6,9,13-trioxo-7-phenethyl-2-oxa-13,5,8-triaza-1(6,3)- |
| | bicyclo[3.2.0]heptana-3(1,3)-benzenacyclotridecaphane-10-yl)acetamide |
| 300 | benzyl ((7S,10S)-34-methyl-6,9,12-trioxo-7-phenethyl-2-oxa-5,8-diaza- |
| | 13(3,1)-piperidina-3(1,3)-benzena-1(1,3)-cyclobutanatridecaphane-10- |
| | yl)carbamate |
| 301 | benzyl ((7S,10S)-3⁴-methyl-6,9,13-trioxo-7-phenethyl-2-oxa-5,8-diaza- |
| | 14(3,1)-piperidina-3(1,3)-benzena-1(1,3)-cyclobutanatetradecaphane-10- |
| | yl)carbamate |
| 302 | 2-(2-aminothiazol-4-yl)-N-((9S,12S)-5⁴-methyl-8,11,14-trioxo-9-phenethyl- |
| | 4-oxa-7,10-diaza-1(3,1)-piperidina-5(1,3)-benzenacyclotetradecaphane- |
| | 12-yl)acetamide |
| 303 | 2-(4-amino-3-methylphenyl)-N-((9S,12S)-5⁴-methyl-8,11,14-trioxo-9- |
| | phenethyl-4-oxa-7,10-diaza-1(3,1)-piperidina-5(1,3)- |
| | benzenacyclotetradecaphane-12-yl)acetamide |
| 304 | 2-(4-amino-2-methylphenyl)-N-((9S,12S)-5⁴-methyl-8,11,14-trioxo-9- |
| | phenethyl-4-oxa-7,10-diaza-1(3,1)-piperidina-5(1,3)- |
| | benzenacyclotetradecaphane-12-yl)acetamide |
| 305 | 1-(4-aminophenyl)-N-((9S,12S)-5⁴-methyl-8,11,14-trioxo-9-phenethyl-4- |
| | oxa-7,10-diaza-1(3,1)-piperidina-5(1,3)-benzenacyclotetradecaphane-12- |
| | yl)cyclopentane-1-carboxamide |
| 306 | 1-(4-aminophenyl)-N-((9S,12S)-5⁴-methyl-8,11,14-trioxo-9-phenethyl-4- |
| | oxa-7,10-diaza-1(3,1)-piperidina-5(1,3)-benzenacyclotetradecaphane-12- |
| | yl)cyclobutane-1-carboxamide |
| 307 | 1-(4-aminophenyl)-N-((9S,12S)-5⁴-methyl-8,11,14-trioxo-9-phenethyl-4- |
| | oxa-7,10-diaza-1(3,1)-piperidina-5(1,3)-benzenacyclotetradecaphane-12- |
| | yl)cyclopropane-1-carboxamide |
| 308 | 2-(4-aminophenyl)-2-methyl-N-((9S,12S)-5⁴-methyl-8,11,14-trioxo-9- |
| | phenethyl-4-oxa-7,10-diaza-1(3,1)-piperidina-5(1,3)- |
| | benzenacyclotetradecaphane-12-yl)propanamide |
| 309 | 2-(4-aminophenyl)-N-((9S,12S)-5⁴-methyl-8,11,14-trioxo-9-phenethyl-4- |
| | oxa-7,10-diaza-1(3,1)-piperidina-5(1,3)-benzenacyclotetradecaphane-12- |
| | yl)propanamide |
| 310 | 2-(5-amino-1,3,4-thiadiazol-2-yl)-N-((9S,12S)-5⁴-methyl-8,11,14-trioxo-9- |
| | phenethyl-4-oxa-7,10-diaza-1(3,1)-piperidina-5(1,3)- |
| | benzenacyclotetradecaphane-12-yl)acetamide |
| 311 | 2-(2-aminothiazol-5-yl)-N-((9S,12S)-5⁴-methyl-8,11,14-trioxo-9-phenethyl- |
| | 4-oxa-7,10-diaza-1(3,1)-piperidina-5(1,3)-benzenacyclotetradecaphane- |
| | 12-yl)acetamide |
| 312 | benzyl ((8S,11S)-1⁴,5-dimethyl-6,9,12-trioxo-11-phenethyl-2-oxa-5,10,13- |
| | triaza-1(1,3)-benzenacyclotetradecaphane-8-yl)carbamate |
| 313 | benzyl ((9S,12S)-5⁴-methyl-8,11,14-trioxo-9-phenethyl-11,12,13,14- |
| | tetrahydro-4-oxa-7,10-diaza-1(3,1)-quinolina-5(1,3)- |
| | benzenacyclotetradecaphane-12-yl)carbamate |
| 314 | benzyl ((9S,12S)-5⁴-methyl-8,11,14-trioxo-9-phenethyl- |
| | 11,12,13,14,14a,15,16,17,18,18a-decahydro-4-oxa-7,10-diaza-1(3,1)- |
| | quinolina-5(1,3)-benzenacyclotetradecaphane-12-yl)carbamate |
| 315 | benzyl ((9S,12S)-1⁶,5⁴-dimethyl-8,11,14-trioxo-9-phenethyl-4-oxa-7,10- |
| | diaza-1(3,1)-piperidina-5(1,3)-benzenacyclotetradecaphane-12- |
| | yl)carbamate |
| 316 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-1,2,3,4- |
| | tetrahydroisoquinoline-5-carboxamide |
| 317 | 5-amino-N-((9S,12S)-5⁴-methyl-8,11,14-trioxo-9-phenethyl-4-oxa-7,10- |
| | diaza-1(3,1)-piperidina-5(1,3)-benzenacyclotetradecaphane-12-yl)-2,3- |
| | dihydro-1H-indene-1-carboxamide |
| 318 | 2-(4-amino-2-fluorophenyl)-N-((9S,12S)-5⁴-methyl-8,11,14-trioxo-9- |
| | phenethyl-4-oxa-7,10-diaza-1(3,1)-piperidina-5(1,3)- |
| | benzenacyclotetradecaphane-12-yl)acetamide |
| 319 | 3-amino-N-((9S,12S)-5⁴-methyl-8,11,14-trioxo-9-phenethyl-4-oxa-7,10- |
| | diaza-1(3,1)-piperidina-5(1,3)-benzenacyclotetradecaphane-12- |
| | yl)isoquinoline-8-carboxamide |
| 320 | 2-(4-amino-3,5-dichlorophenyl)-N-((9S,12S)-54-methyl-8,11,14-trioxo-9- |
| | phenethyl-4-oxa-7,10-diaza-1(3,1)-piperidina-5(1,3)- |
| | benzenacyclotetradecaphane-12-yl)acetamide |
| 321 | benzyl ((9S,12S)-5⁴-methyl-8,11,14-trioxo-9-phenethyl-4-oxa-7,10-diaza- |
| | 1(3,1)-piperidina-5(1,3)-naphthalenacyclotetradecaphane-12-yl)carbamate |
| 322 | benzyl ((12S,15S)-1⁴-methyl-8,13,16-trioxo-15-phenethyl-2-oxa-7,14,17- |
| | triaza-1(1,3)-benzenacyclooctadecaphane-12-yl)carbamate |
| 323 | 2-(4-acetamidophenyl)-2-methoxy-N-((9S,12S)-5⁴-methyl-8,11,14-trioxo-9- |
| | phenethyl-4-oxa-7,10-diaza-1(3,1)-piperidina-5(1,3)- |
| | benzenacyclotetradecaphane-12-yl)acetamide |
| 324 | 2-(4-acetamidophenyl)-2-ethoxy-N-((9S,12S)-5⁴-methyl-8,11,14-trioxo-9- |
| | phenethyl-4-oxa-7,10-diaza-1(3,1)-piperidina-5(1,3)- |
| | benzenacyclotetradecaphane-12-yl)acetamide |
| 325 | benzyl ((9S,12S)-54-methyl-8,11,14-trioxo-9-phenethyl-4-oxa-7,10-diaza- |
| | 1(3,1)-pyrrolidina-5(1,3)-benzenacyclotetradecaphane-12-yl)carbamate |
| 326 | 2-(4-aminophenyl)-2-methoxy-N-((9S,12S)-5⁴-methyl-8,11,14-trioxo-9- |
| | phenethyl-4-oxa-7,10-diaza-1(3,1)-piperidina-5(1,3)- |
| | benzenacyclotetradecaphane-12-yl)acetamide |
| 327 | N-((9S,12R)-5⁴-methyl-8,11,14-trioxo-9-phenethyl-4-oxa-7,10-diaza- |
| | 1(3,1)-piperidina-5(1,3)-benzenacyclotetradecaphane-12-yl)-2-(3-(pyridin- |
| | 3-yl)-1H-1,2,4-triazol-5-yl)acetamide |
| 328 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-2-(3-phenyl- |
| | 1H-1,2,4-triazol-5-yl)acetamide |
| 329 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-2-(3-(pyridin- |
| | 4-yl)-1H-1,2,4-triazol-5-yl)acetamide |
| 330 | 2-(imidazo[2,1-b]thiazol-6-yl)-N-((9S,12S)-5⁴-methyl-8,11,14-trioxo-9- |
| | phenethyl-4-oxa-7,10-diaza-1(3,1)-piperidina-5(1,3)- |
| | benzenacyclotetradecaphane-12-yl)acetamide |
| 331 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-2-(pyridin-2- |
| | yl)acetamide |
| 332 | 2-(3-methyl-1H-1,2,4-triazol-5-yl)-N-((9S,12S)-5⁴-methyl-8,11,14-trioxo-9- |
| | phenethyl-4-oxa-7,10-diaza-1(3,1)-piperidina-5(1,3)- |
| | benzenacyclotetradecaphane-12-yl)acetamide |
| 333 | 2-(2-fluorophenyl)-N-((9S,12S)-5⁴-methyl-8,11,14-trioxo-9-phenethyl-4- |
| | oxa-7,10-diaza-1(3,1)-piperidina-5(1,3)-benzenacyclotetradecaphane-12- |
| | yl)acetamide |
| 334 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-2-(5-oxo-1- |
| | phenyl-4,5-dihydro-1H-pyrazol-3-yl)acetamide |
| 335 | 2-(5-hydroxyisoxazol-3-yl)-N-((9S,12S)-5⁴-methyl-8,11,14-trioxo-9- |
| | phenethyl-4-oxa-7,10-diaza-1(3,1)-piperidina-5(1,3)- |
| | benzenacyclotetradecaphane-12-yl)acetamide |
| 336 | benzyl ((12S)-5⁴-methyl-8,11,14-trioxo-9-(2-(pyridin-4-yl)ethyl)-4-oxa-7,10- |
| | diaza-1(3,1)-piperidina-5(1,3)-benzenacyclotetradecaphane-12- |
| | yl)carbamate |
| 337 | benzyl ((9S,12S)-1⁵,5⁴-dimethyl-8,11,14-trioxo-9-phenethyl-4-oxa-7,10- |
| | diaza-1(3,1)-piperidina-5(1,3)-benzenacyclotetradecaphane-12- |
| | yl)carbamate |
| 338 | N-((12S)-9-(2-(1-acetylpiperidin-4-yl)ethyl)-5⁴-methyl-8,11,14-trioxo-4-oxa- |
| | 7,10-diaza-1(3,1)-piperidina-5(1,3)-benzenacyclotetradecaphane-12- |
| | yl)acetamide |
| 339 | 6-amino-N-((9S,12S)-5⁴-methyl-8,11,14-trioxo-9-phenethyl-4-oxa-7,10- |
| | diaza-1(3,1)-piperidina-5(1,3)-benzenacyclotetradecaphane-12-yl)-2- |
| | naphthamide |
| 340 | 2-(5-amino-1H-1,2,4-triazol-3-yl)-N-((9S,12S)-5⁴-methyl-8,11,14-trioxo-9- |
| | phenethyl-4-oxa-7,10-diaza-1(3,1)-piperidina-5(1,3)- |
| | benzenacyclotetradecaphane-12-yl)acetamide |
| 341 | 2-(4-acetylphenyl)-N-((9S,12S)-5⁴-methyl-8,11,14-trioxo-9-phenethyl-4- |
| | oxa-7,10-diaza-1(3,1)-piperidina-5(1,3)-benzenacyclotetradecaphane-12- |
| | yl)acetamide |
| 342 | 2-(5-amino-1,3,4-thiadiazol-2-yl)-N-((12S)-5⁴-methyl-8,11,14-trioxo-9-(2- |
| | (pyridin-4-yl)ethyl)-4-oxa-7,10-diaza-1(3,1)-piperidina-5(1,3)- |
| | benzenacyclotetradecaphane-12-yl)acetamide |
| 343 | 1-(4-aminophenyl)-N-((12S)-5⁴-methyl-8,11,14-trioxo-9-(2-(pyridin-4- |
| | yl)ethyl)-4-oxa-7,10-diaza-1(3,1)-piperidina-5(1,3)- |
| | benzenacyclotetradecaphane-12-yl)cyclopropane-1-carboxamide |
| 344 | 2-(4-amino-2-fluorophenyl)-N-((12S)-5⁴-methyl-8,11,14-trioxo-9-(2- |
| | (pyridin-4-yl)ethyl)-4-oxa-7,10-diaza-1(3,1)-piperidina-5(1,3)- |
| | benzenacyclotetradecaphane-12-yl)acetamide |
| 345 | 2-(4-aminophenyl)-2-ethoxy-N-((9S,12S)-5⁴-methyl-8,11,14-trioxo-9- |
| | phenethyl-4-oxa-7,10-diaza-1(3,1)-piperidina-5(1,3)- |
| | benzenacyclotetradecaphane-12-yl)acetamide |
| 347 | benzyl ((13S,16S)-1⁴-methyl-8,14,17-trioxo-16-phenethyl-2-oxa-7,15,18- |
| | triaza-1(1,3)-benzenacyclononadecaphane-13-yl)carbamate |
| 348 | 3-mercapto-2,2-dimethyl-N-((9S,12S)-5⁴-methyl-8,11,14-trioxo-9- |
| | phenethyl-4-oxa-7,10-diaza-1(3,1)-piperidina-5(1,3)- |
| | benzenacyclotetradecaphane-12-yl)propanamide |
| 349 | benzyl ((8S,11S)-4⁴-methyl-7,10,14-trioxo-8-phenethyl-3-oxa-6,9-diaza- |
| | 1(3,1)-pyrrolidina-4(1,3)-benzenacyclotetradecaphane-11-yl)carbamate |
| 350 | benzyl ((9S,12S)-5⁴-methyl-8,11,15-trioxo-9-phenethyl-4-oxa-7,10-diaza- |
| | 1(3,1)-pyrrolidina-5(1,3)-benzenacyclopentadecaphane-12-yl)carbamate |
| 351 | benzyl ((8S,11S)-4⁴-methyl-7,10,15-trioxo-8-phenethyl-3-oxa-6,9-diaza- |
| | 1(3,1)-pyrrolidina-4(1,3)-benzenacyclopentadecaphane-11-yl)carbamate |
| 352 | benzyl ((9S,12S)-5⁴-methyl-8,11,16-trioxo-9-phenethyl-4-oxa-7,10-diaza- |
| | 1(3,1)-pyrrolidina-5(1,3)-benzenacyclohexadecaphane-12-yl)carbamate |
| 353 | benzyl ((9S,12S)-5⁴-methyl-8,11,17-trioxo-9-phenethyl-4-oxa-7,10-diaza- |
| | 1(3,1)-azetidina-5(1,3)-benzenacycloheptadecaphane-12-yl)carbamate |
| 354 | benzyl ((12S,15S)-1⁴,1⁵-dimethyl-8,13,16-trioxo-15-phenethyl-2-oxa- |
| | 7,14,17-triaza-1(1,3)-benzenacyclooctadecaphane-12-yl)carbamate |
| 355 | 5⁴,5⁵-dimethyl-9-(1-methyl-1H-pyrazol-4-yl)-4-oxa-7,10-diaza-1(3,1)- |
| | piperidina-5(1,3)-benzenacyclotetradecaphane-8,11,14-trione |
| 356 | 5⁴-methyl-9-(1-methyl-1H-pyrazol-4-yl)-4-oxa-7,10-diaza-1(3,1)-piperidina- |
| | 5(1,3)-benzenacyclohexadecaphane-8,11,16-trione |
| 358 | N-((12S)-5⁴-methyl-9-(1-methyl-1H-pyrazol-4-yl)-8,11,14-trioxo-4-oxa- |
| | 7,10-diaza-1(3,1)-piperidina-5(1,3)-benzenacyclotetradecaphane-12-yl)-2- |
| | (pyrrolidin-1-yl)acetamide |
| 359 | (2S)-N-((12S)-5⁴-methyl-9-(1-methyl-1H-pyrazol-4-yl)-8,11,14-trioxo-4- |
| | oxa-7,10-diaza-1(3,1)-piperidina-5(1,3)-benzenacyclotetradecaphane-12- |
| | yl)-2-phenylpropanamide |
| 360 | N-((12S)-5⁴-methyl-9-(1-methyl-1H-pyrazol-4-yl)-8,11,14-trioxo-4-oxa- |
| | 7,10-diaza-1(3,1)-piperidina-5(1,3)-benzenacyclotetradecaphane-12-yl)- |
| | 1,2,3,4-tetrahydronaphthalene-1-carboxamide |
| 361 | (2R)-2-methoxy-N-((12S)-5⁴-methyl-9-(1-methyl-1H-pyrazol-4-yl)-8,11,14- |
| | trioxo-4-oxa-7,10-diaza-1(3,1)-piperidina-5(1,3)- |
| | benzenacyclotetradecaphane-12-yl)-2-phenylacetamide |
| 362 | 2-(3-methoxyphenyl)-N-((12S)-5⁴-methyl-9-(1-methyl-1H-pyrazol-4-yl)- |
| | 8,11,14-trioxo-4-oxa-7,10-diaza-1(3,1)-piperidina-5(1,3)- |
| | benzenacyclotetradecaphane-12-yl)acetamide |
| 363 | Ethyl ((12S)-5⁴-methyl-9-(1-methyl-1H-pyrazol-4-yl)-8,11,14-trioxo-4-oxa- |
| | 7,10-diaza-1(3,1)-piperidina-5(1,3)-benzenacyclotetradecaphane-12- |
| | yl)carbamate |
| 364 | N-((12S,15S)-1⁴-methyl-8,13,16-trioxo-15-phenethyl-2-oxa-7,14,17-triaza- |
| | 1(1,3)-benzenacyclooctadecaphane-12-yl)acetamide |
| 369 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-2-(quinolin-6- |
| | yl)propanamide |
| 371 | 1-(4-aminophenyl)-N-((9S,12S)-5⁴-methyl-8,11,14-trioxo-9-(2-(pyrazin-2- |
| | yl)ethyl)-4-oxa-7,10-diaza-1(3,1)-piperidina-5(1,3)- |
| | benzenacyclotetradecaphane-12-yl)cyclopropane-1-carboxamide |
| 372 | 2-(4-amino-2-fluorophenyl)-N-((9S,12S)-5⁴-methyl-8,11,14-trioxo-9-(2- |
| | (pyrazin-2-yl)ethyl)-4-oxa-7,10-diaza-1(3,1)-piperidina-5(1,3)- |
| | benzenacyclotetradecaphane-12-yl)acetamide |
| 373 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-2-(o- |
| | tolyl)acetamide |
| 387 | tert-butyl (4-(2-(((9S,12S)-5⁴-methyl-8,11,14-trioxo-9-phenethyl-4-oxa- |
| | 7,10-diaza-1(3,1)-piperidina-5(1,3)-benzenacyclotetradecaphane-12- |
| | yl)amino)-2-oxoethyl)thiazol-2-yl)carbamate |
| 388 | tert-butyl (2-methyl-4-(2-(((9S,12S)-5⁴-methyl-8,11,14-trioxo-9-phenethyl- |
| | 4-oxa-7,10-diaza-1(3,1)-piperidina-5(1,3)-benzenacyclotetradecaphane- |
| | 12-yl)amino)-2-oxoethyl)phenyl)carbamate |
| 389 | tert-butyl (3-methyl-4-(2-(((9S,12S)-5⁴-methyl-8,11,14-trioxo-9-phenethyl- |
| | 4-oxa-7,10-diaza-1(3,1)-piperidina-5(1,3)-benzenacyclotetradecaphane- |
| | 12-yl)amino)-2-oxoethyl)phenyl)carbamate |
| 390 | tert-butyl (4-(1-(((9S,12S)-5⁴-methyl-8,11,14-trioxo-9-phenethyl-4-oxa- |
| | 7,10-diaza-1(3,1)-piperidina-5(1,3)-benzenacyclotetradecaphane-12- |
| | yl)carbamoyl)cyclopentyl)phenyl)carbamate |
| 391 | tert-butyl (4-(1-(((9S,12S)-5⁴-methyl-8,11,14-trioxo-9-phenethyl-4-oxa- |
| | 7,10-diaza-1(3,1)-piperidina-5(1,3)-benzenacyclotetradecaphane-12- |
| | yl)carbamoyl)cyclobutyl)phenyl)carbamate |
| 392 | tert-butyl (4-(1-(((9S,12S)-5⁴-methyl-8,11,14-trioxo-9-phenethyl-4-oxa- |
| | 7,10-diaza-1(3,1)-piperidina-5(1,3)-benzenacyclotetradecaphane-12- |
| | yl)carbamoyl)cyclopropyl)phenyl)carbamate |
| 393 | tert-butyl (4-(2-methyl-1-(((9S,12S)-5⁴-methyl-8,11,14-trioxo-9-phenethyl- |
| | 4-oxa-7,10-diaza-1(3,1)-piperidina-5(1,3)-benzenacyclotetradecaphane- |
| | 12-yl)amino)-1-oxopropan-2-yl)phenyl)carbamate |
| 394 | 2-(4-aminophenyl)-N-((9S,12S)-5⁴-methyl-8,11,14-trioxo-9-phenethyl-4- |
| | oxa-7,10-diaza-1(3,1)-piperidina-5(1,3)-benzenacyclotetradecaphane-12- |
| | yl)propanamide |
| 395 | tert-butyl (5-(2-(((9S,12S)-5⁴-methyl-8,11,14-trioxo-9-phenethyl-4-oxa- |
| | 7,10-diaza-1(3,1)-piperidina-5(1,3)-benzenacyclotetradecaphane-12- |
| | yl)amino)-2-oxoethyl)-1,3,4-thiadiazol-2-yl)carbamate |
| 396 | tert-butyl (5-(2-(((9S,12S)-5⁴-methyl-8,11,14-trioxo-9-phenethyl-4-oxa- |
| | 7,10-diaza-1(3,1)-piperidina-5(1,3)-benzenacyclotetradecaphane-12- |
| | yl)amino)-2-oxoethyl)thiazol-2-yl)carbamate |
| 402 | benzyl ((9S,12S)-1⁴,5-dimethyl-6,10,13-trioxo-12-phenethyl-2-oxa- |
| | 5,11,14-triaza-1(1,3)-benzenacyclopentadecaphane-9-yl)carbamate |
| 405 | benzyl ((10S,13S)-1⁴,5-dimethyl-6,11,14-trioxo-13-phenethyl-2-oxa- |
| | 5,12,15-triaza-1(1,3)-benzenacyclohexadecaphane-10-yl)carbamate |
| 419 | tert-butyl 8-(((9S,12S)-5⁴-methyl-8,11,14-trioxo-9-phenethyl-4-oxa-7,10- |
| | diaza-1(3,1)-piperidina-5(1,3)-benzenacyclotetradecaphane-12- |
| | yl)carbamoyl)-3,4-dihydroisoquinoline-2(1H)-carboxylate |
| 420 | tert-butyl (1-(((9S,12S)-5⁴-methyl-8,11,14-trioxo-9-phenethyl-4-oxa-7,10- |
| | diaza-1(3,1)-piperidina-5(1,3)-benzenacyclotetradecaphane-12- |
| | yl)carbamoyl)-2,3-dihydro-1H-inden-5-yl)carbamate |
| 421 | tert-butyl (3-fluoro-4-(2-(((9S,12S)-5⁴-methyl-8,11,14-trioxo-9-phenethyl-4- |
| | oxa-7,10-diaza-1(3,1)-piperidina-5(1,3)-benzenacyclotetradecaphane-12- |
| | yl)amino)-2-oxoethyl)phenyl)carbamate |
| 422 | tert-butyl (8-(((9S,12S)-5⁴-methyl-8,11,14-trioxo-9-phenethyl-4-oxa-7,10- |
| | diaza-1(3,1)-piperidina-5(1,3)-benzenacyclotetradecaphane-12- |
| | yl)carbamoyl)isoquinolin-3-yl)carbamate |
| 423 | tert-butyl (2,6-dichloro-4-(2-(((9S,12S)-5⁴-methyl-8,11,14-trioxo-9- |
| | phenethyl-4-oxa-7,10-diaza-1(3,1)-piperidina-5(1,3)- |
| | benzenacyclotetradecaphane-12-yl)amino)-2-oxoethyl)phenyl)carbamate |
| 429 | tert-butyl (4-(1-methoxy-2-(((9S,12S)-5⁴-methyl-8,11,14-trioxo-9- |
| | phenethyl-4-oxa-7,10-diaza-1(3,1)-piperidina-5(1,3)- |
| | benzenacyclotetradecaphane-12-yl)amino)-2-oxoethyl)phenyl)carbamate |
| 437 | ((12S)-12-amino-5⁴-methyl-8,11,14-trioxo-9-(2-(pyridin-4-yl)ethyl)-4-oxa- |
| | 7,10-diaza-1(3,1)-piperidina-5(1,3)-benzenacyclotetradecaphane |
| 438 | N-((12S)-12-amino-9-(2-(1-acetylpiperidin-4-yl)ethyl)-5⁴-methyl-8,11,14- |
| | trioxo-4-oxa-7,10-diaza-1(3,1)-piperidina-5(1,3)- |
| | benzenacyclotetradecaphane |
| 439 | tert-butyl (5-(2-(((12S)-5⁴-methyl-8,11,14-trioxo-9-(2-(pyridin-4-yl)ethyl)-4- |
| | oxa-7,10-diaza-1(3,1)-piperidina-5(1,3)-benzenacyclotetradecaphane-12- |
| | yl)amino)-2-oxoethyl)-1,3,4-thiadiazol-2-yl)carbamate |
| 440 | tert-butyl (6-(((9S,12S)-5⁴-methyl-8,11,14-trioxo-9-phenethyl-4-oxa-7,10- |
| | diaza-1(3,1)-piperidina-5(1,3)-benzenacyclotetradecaphane-12- |
| | yl)carbamoyl)naphthalen-2-yl)carbamate |
| 441 | tert-butyl (5-(2-(((9S,12S)-5⁴-methyl-8,11,14-trioxo-9-phenethyl-4-oxa- |
| | 7,10-diaza-1(3,1)-piperidina-5(1,3)-benzenacyclotetradecaphane-12- |
| | yl)amino)-2-oxoethyl)-4H-1,2,4-triazol-3-yl)carbamate |
| 442 | tert-butyl (4-(1-ethoxy-2-(((9S,12S)-5⁴-methyl-8,11,14-trioxo-9-phenethyl- |
| | 4-oxa-7,10-diaza-1(3,1)-piperidina-5(1,3)-benzenacyclotetradecaphane- |
| | 12-yl)amino)-2-oxoethyl)phenyl)carbamate |
| 443 | tert-butyl (4-(1-(((12S)-5⁴-methyl-8,11,14-trioxo-9-(2-(pyridin-4-yl)ethyl)-4- |
| | oxa-7,10-diaza-1(3,1)-piperidina-5(1,3)-benzenacyclotetradecaphane-12- |
| | yl)carbamoyl)cyclopropyl)phenyl)carbamate |
| 444 | tert-butyl (3-fluoro-4-(2-(((12S)-5⁴-methyl-8,11,14-trioxo-9-(2-(pyridin-4- |
| | yl)ethyl)-4-oxa-7,10-diaza-1(3,1)-piperidina-5(1,3)- |
| | benzenacyclotetradecaphane-12-yl)amino)-2-oxoethyl)phenyl)carbamate |
| 479 | benzyl ((12S)-5⁴-methyl-9-(1-methyl-1H-pyrazol-4-yl)-8,11,14-trioxo-4- |
| | oxa-7,10-diaza-1(3,1)-piperidina-5(1,3)-benzenacyclotetradecaphane-12- |
| | yl)carbamate |
| 480 | (12S)-12-amino-5⁴-methyl-9-(1-methyl-1H-pyrazol-4-yl)-4-oxa-7,10-diaza- |
| | 1(3,1)-piperidina-5(1,3)-benzenacyclotetradecaphane-8,11,14-trione |
| 495 | 54-methyl-9-phenethyl-4-oxa-7,10-diaza-1(3,1)-piperidina-5(1,3)- |
| | benzenacyclotetradecaphane-8,11,14-trione |
| 511 | benzyl ((12S)-9-(2-(1,5-naphthyridin-3-yl)ethyl)-5⁴-methyl-8,11,14-trioxo-4- |
| | oxa-7,10-diaza-1(3,1)-piperidina-5(1,3)-benzenacyclotetradecaphane-12- |
| | yl)carbamate |
| 503 | benzyl ((12S)-9-phenethyl-5⁴-methyl-8,11,14-trioxo-4-oxa-7,10-diaza- |
| | 1(3,1)-piperidina-5(1,3)-benzenacyclotetradecaphane-12-yl)carbamate |
| 505 | benzyl ((12S)-5⁴-methyl-8,11,14-trioxo-9-(2-(pyrido[2,3-b]pyrazin-7- |
| | yl)ethyl)-4-oxa-7,10-diaza-1(3,1)-piperidina-5(1,3)- |
| | benzenacyclotetradecaphane-12-yl)carbamate |
| 509 | 4-(2-((12S)-12-(((benzyloxy)carbonyl)amino)-5⁴-methyl-8,11,14-trioxo-4- |
| | oxa-7,10-diaza-1(3,1)-piperidina-5(1,3)-benzenacyclotetradecaphane-9- |
| | yl)ethyl)benzenesulfonic acid |
| 510 | benzyl ((12S)-5⁴-methyl-9-(4-(3-methyloxetan-3-yl)phenethyl)-8,11,14- |
| | trioxo-4-oxa-7,10-diaza-1(3,1)-piperidina-5(1,3)- |
| | benzenacyclotetradecaphane-12-yl)carbamate |
| 512 | benzyl ((12S)-5⁴-methyl-9-(2-methyloxazol-4-yl)-8,11,14-trioxo-4-oxa- |
| | 7,10-diaza-1(3,1)-piperidina-5(1,3)-benzenacyclotetradecaphane-12- |
| | yl)carbamate |
| 513 | methyl (((12R)-12-(((benzyloxy)carbonyl)amino)-5⁴-methyl-8,11,14-trioxo- |
| | 4-oxa-7,10-diaza-1(3,1)-piperidina-5(1,3)-benzenacyclotetradecaphane-9- |
| | yl)methyl)(phenyl)carbamate |
| 549 | N-((12S)-5⁴-methyl-8,11,14-trioxo-9-(2-(pyridin-3-yl)ethyl)-4-oxa-7,10- |
| | diaza-1(3,1)-piperidina-5(1,3)-benzenacyclotetradecaphane-12- |
| | yl)acetamide |
| 550 | N-((12S)-5⁴-methyl-8,11,14-trioxo-9-phenethyl-4-oxa-7,10-diaza-1(3,1)- |
| | piperidina-5(1,3)-benzenacyclotetradecaphane-12-yl)acetamide |
| 551 | N-((12S)-5⁴-methyl-8,11,14-trioxo-9-(2-(pyridin-4-yl)ethyl)-4-oxa-7,10- |
| | diaza-1(3,1)-piperidina-5(1,3)-benzenacyclotetradecaphane-12- |
| | yl)acetamide |
| 552 | N-((12S)-5⁴-methyl-9-(1-methyl-1H-pyrazol-4-yl)-8,11,14-trioxo-4-oxa- |
| | 7,10-diaza-1(3,1)-piperidina-5(1,3)-benzenacyclotetradecaphane-12- |
| | yl)acetamide |
| 553 | N-((12S)-5⁴-methyl-9-(2-methyloxazol-4-yl)-8,11,14-trioxo-4-oxa-7,10- |
| | diaza-1(3,1)-piperidina-5(1,3)-benzenacyclotetradecaphane-12- |
| | yl)acetamide |
| 554 | N-((12S)-5⁴-methyl-8,11,14-trioxo-9-(quinolin-6-yl)-4-oxa-7,10-diaza- |
| | 1(3,1)-piperidina-5(1,3)-benzenacyclotetradecaphane-12-yl)acetamide |
| 555 | N-((12S)-5⁴-methyl-8,11,14-trioxo-9-((2-oxobenzo[d]oxazol-3(2H)- |
| | yl)methyl)-4-oxa-7,10-diaza-1(3,1)-piperidina-5(1,3)- |
| | benzenacyclotetradecaphane-12-yl)acetamide |
| 556 | N-((12S)-5⁴-methyl-9-(5-methylisothiazol-3-yl)-8,11,14-trioxo-4-oxa-7,10- |
| | diaza-1(3,1)-piperidina-5(1,3)-benzenacyclotetradecaphane-12- |
| | yl)acetamide |
| 557 | N-((12S)-5⁴-methyl-9-(1-methyl-1H-imidazol-4-yl)-8,11,14-trioxo-4-oxa- |
| | 7,10-diaza-1(3,1)-piperidina-5(1,3)-benzenacyclotetradecaphane-12- |
| | yl)acetamide |
| 558 | N-((12S)-9-benzyl-5⁴-methyl-8,11,14-trioxo-4-oxa-7,10-diaza-1(3,1)- |
| | piperidina-5(1,3)-benzenacyclotetradecaphane-12-yl)acetamide |
| 559 | N-((12S)-5⁴-methyl-8,11,14-trioxo-9-(1-phenyl-1H-pyrazol-4-yl)-4-oxa- |
| | 7,10-diaza-1(3,1)-piperidina-5(1,3)-benzenacyclotetradecaphane-12- |
| | yl)acetamide |
| 560 | methyl (((12S)-12-acetamido-5⁴-methyl-8,11,14-trioxo-4-oxa-7,10-diaza- |
| | 1(3,1)-piperidina-5(1,3)-benzenacyclotetradecaphane-9- |
| | yl)methyl)(phenyl)carbamate |
| 561 | N-((12S)-9-(1,5-dimethyl-1H-pyrazol-3-yl)-5⁴-methyl-8,11,14-trioxo-4-oxa- |
| | 7,10-diaza-1(3,1)-piperidina-5(1,3)-benzenacyclotetradecaphane-12- |
| | yl)acetamide |
| 562 | N-((12S)-5⁴-methyl-8,11,14-trioxo-9-(1H-pyrazol-4-yl)-4-oxa-7,10-diaza- |
| | 1(3,1)-piperidina-5(1,3)-benzenacyclotetradecaphane-12-yl)acetamide |
| 563 | N-((12S)-5⁴-methyl-8,11,14-trioxo-9-(2-(thiazol-2-yl)ethyl)-4-oxa-7,10- |
| | diaza-1(3,1)-piperidina-5(1,3)-benzenacyclotetradecaphane-12- |
| | yl)acetamide |
| 564 | N-((12S)-5⁴-methyl-8,11,14-trioxo-9-(2-(pyrazin-2-yl)ethyl)-4-oxa-7,10- |
| | diaza-1(3,1)-piperidina-5(1,3)-benzenacyclotetradecaphane-12- |
| | yl)acetamide |
| 565 | N-((12S)-9-(1-benzyl-1H-pyrazol-4-yl)-5⁴-methyl-8,11,14-trioxo-4-oxa- |
| | 7,10-diaza-1(3,1)-piperidina-5(1,3)-benzenacyclotetradecaphane-12- |
| | yl)acetamide |
| 566 | N-((12S)-9-(1-(2-(2-(2-(2-aminoethoxy)ethoxy)ethoxy)ethyl)-1H-pyrazol-4- |
| | yl)-5⁴-methyl-8,11,14-trioxo-4-oxa-7,10-diaza-1(3,1)-piperidina-5(1,3)- |
| | benzenacyclotetradecaphane-12-yl)acetamide |
| 567 | 3-(4-((12S)-12-acetamido-5⁴-methyl-8,11,14-trioxo-4-oxa-7,10-diaza- |
| | 1(3,1)-piperidina-5(1,3)-benzenacyclotetradecaphane-9-yl)-1H-pyrazol-1- |
| | yl)propanoic acid |
| 568 | 4-(4-((12S)-12-acetamido-5⁴-methyl-8,11,14-trioxo-4-oxa-7,10-diaza- |
| | 1(3,1)-piperidina-5(1,3)-benzenacyclotetradecaphane-9-yl)-1H-pyrazol-1- |
| | yl)butanoic acid |
| 570 | N-((12S)-9-(1-(3-mercaptopropyl)-1H-pyrazol-4-yl)-5⁴-methyl-8,11,14- |
| | trioxo-4-oxa-7,10-diaza-1(3,1)-piperidina-5(1,3)- |
| | benzenacyclotetradecaphane-12-yl)acetamide |
| 571 | 3-(4-((12S)-12-acetamido-5⁴-methyl-8,11,14-trioxo-4-oxa-7,10-diaza- |
| | 1(3,1)-piperidina-5(1,3)-benzenacyclotetradecaphane-9-yl)-1H-pyrazol-1- |
| | yl)propane-1-sulfonic acid |
| 572 | N-(4-((12S)-12-acetamido-5⁴-methyl-8,11,14-trioxo-4-oxa-7,10-diaza- |
| | 1(3,1)-piperidina-5(1,3)-benzenacyclotetradecaphane-9-yl)-1H-pyrazol-1- |
| | yl)ethane-1-sulfonic acid |
| 573 | 3-hydroxy-2,2-dimethyl-N-((13R,9S,12S)-5⁴-methyl-8,11,14-trioxo-9- |
| | phenethyl-4-oxa-7,10-diaza-1(3,1)-piperidina-5(1,3)- |
| | benzenacyclotetradecaphane-12-yl)propanamide |
| 574 | (4S)-4-amino-5-((3-methyl-4-(2-(((9S,12S)-5⁴-methyl-8,11,14-trioxo-9- |
| | phenethyl-4-oxa-7,10-diaza-1(3,1)-piperidina-5(1,3)- |
| | benzenacyclotetradecaphane-12-yl)amino)-2-oxoethyl)phenyl)amino)-5- |
| | oxopentanoic acid |
| 575 | N-((9S,12S)-9-(2-cyclohexylethyl)-5⁴-methyl-8,11,14-trioxo-4-oxa-7,10- |
| | diaza-1(3,1)-piperidina-5(1,3)-benzenacyclotetradecaphane-12-yl)-2- |
| | (imidazo[2,1-b]thiazol-6-yl)acetamide |
| 576 | N-(3-(((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)amino)-3- |
| | oxopropyl)-3-oxaspiro[5.5]undecane-9-carboxamide |
| 577 | tert-butyl 6-(3-(((9S,12S)-5⁴-methyl-8,11,14-trioxo-9-phenethyl-4-oxa-7,10- |
| | diaza-1(3,1)-piperidina-5(1,3)-benzenacyclotetradecaphane-12-yl)amino)- |
| | 3-oxopropoxy)-2-azaspiro[3.3]heptane-2-carboxylate |
| 578 | (3S)-3-amino-4-((3-methyl-4-(2-(((9S,12S)-5⁴-methyl-8,11,14-trioxo-9- |
| | phenethyl-4-oxa-7,10-diaza-1(3,1)-piperidina-5(1,3)- |
| | benzenacyclotetradecaphane-12-yl)amino)-2-oxoethyl)phenyl)amino)-4- |
| | oxobutanoic acid |
| 579 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12- |
| | yl)cyclohexanesulfonamide |
| 580 | tert-butyl 6-(((9S,12S)-5⁴-methyl-8,11,14-trioxo-9-phenethyl-4-oxa-7,10- |
| | diaza-1(3,1)-piperidina-5(1,3)-benzenacyclotetradecaphane-12- |
| | yl)carbamoyl)-2-azaspiro[3.3]heptane-2-carboxylate |
| 581 | 3-(3-methoxyphenethoxy)-N-((9S,12S)-5⁴-methyl-8,11,14-trioxo-9- |
| | phenethyl-4-oxa-7,10-diaza-1(3,1)-piperidina-5(1,3)- |
| | benzenacyclotetradecaphane-12-yl)propanamide |
| 582 | 3-(2-methoxyphenethoxy)-N-((9S,12S)-5⁴-methyl-8,11,14-trioxo-9- |
| | phenethyl-4-oxa-7,10-diaza-1(3,1)-piperidina-5(1,3)- |
| | benzenacyclotetradecaphane-12-yl)propanamide |
| 583 | 2-(imidazo[2,1-b]thiazol-6-yl)-N-((9S,12S)-9-isopentyl-5⁴-methyl-8,11,14- |
| | trioxo-4-oxa-7,10-diaza-1(3,1)-piperidina-5(1,3)- |
| | benzenacyclotetradecaphane-12-yl)acetamide |
| 584 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-3- |
| | oxaspiro[5.5]undecane-9-carboxamide |
| 585 | 3-((3-methoxybenzyl)oxy)-N-((9S,12S)-5⁴-methyl-8,11,14-trioxo-9- |
| | phenethyl-4-oxa-7,10-diaza-1(3,1)-piperidina-5(1,3)- |
| | benzenacyclotetradecaphane-12-yl)propanamide |
| 586 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-2-(quinolin-6- |
| | yl)acetamide |
| 587 | 3-(3-methoxyphenoxy)-N-((9S,12S)-5⁴-methyl-8,11,14-trioxo-9-phenethyl- |
| | 4-oxa-7,10-diaza-1(3,1)-piperidina-5(1,3)-benzenacyclotetradecaphane- |
| | 12-yl)propanamid |
| 588 | 3-(2-methoxyphenoxy)-N-((9S,12S)-5⁴-methyl-8,11,14-trioxo-9-phenethyl- |
| | 4-oxa-7,10-diaza-1(3,1)-piperidina-5(1,3)-benzenacyclotetradecaphane- |
| | 12-yl)propanamide |
| 589 | 3-(benzyloxy)-N-((9S,12S)-5⁴-methyl-8,11,14-trioxo-9-phenethyl-4-oxa- |
| | 7,10-diaza-1(3,1)-piperidina-5(1,3)-benzenacyclotetradecaphane-12- |
| | yl)propanamide |
| 590 | 3-((2-oxaspiro[3.3]heptan-6-yl)oxy)-N-((9S,12S)-5⁴-methyl-8,11,14-trioxo- |
| | 9-phenethyl-4-oxa-7,10-diaza-1(3,1)-piperidina-5(1,3)- |
| | benzenacyclotetradecaphane-12-yl)propanamide |
| 591 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-3- |
| | phenoxypropanamide |
| 592 | 1-ethyl-4-fluoro-N-((9S,12S)-5⁴-methyl-8,11,14-trioxo-9-phenethyl-4-oxa- |
| | 7,10-diaza-1(3,1)-piperidina-5(1,3)-benzenacyclotetradecaphane-12- |
| | yl)piperidine-4-carboxamide |
| 593 | 1-cyclohexyl-3-((9S,12S)-5⁴-methyl-8,11,14-trioxo-9-phenethyl-4-oxa- |
| | 7,10-diaza-1(3,1)-piperidina-5(1,3)-benzenacyclotetradecaphane-12- |
| | yl)urea |
| 594 | 3-(cyclopropylmethoxy)-N-((9S,12S)-5⁴-methyl-8,11,14-trioxo-9-phenethyl- |
| | 4-oxa-7,10-diaza-1(3,1)-piperidina-5(1,3)-benzenacyclotetradecaphane- |
| | 12-yl)propanamide |
| 595 | 3-cyclobutoxy-N-((9S,12S)-5⁴-methyl-8,11,14-trioxo-9-phenethyl-4-oxa- |
| | 7,10-diaza-1(3,1)-piperidina-5(1,3)-benzenacyclotetradecaphane-12- |
| | yl)propanamide |
| 596 | 3-(2-methoxyethoxy)-N-((9S,12S)-5⁴-methyl-8,11,14-trioxo-9-phenethyl-4- |
| | oxa-7,10-diaza-1(3,1)-piperidina-5(1,3)-benzenacyclotetradecaphane-12- |
| | yl)propanamide |
| 597 | 3-cyclopropoxy-N-((9S,12S)-5⁴-methyl-8,11,14-trioxo-9-phenethyl-4-oxa- |
| | 7,10-diaza-1(3,1)-piperidina-5(1,3)-benzenacyclotetradecaphane-12- |
| | yl)propanamide |
| 598 | 3-amino-2,2-difluoro-N-((9S,12S)-5⁴-methyl-8,11,14-trioxo-9-phenethyl-4- |
| | oxa-7,10-diaza-1(3,1)-piperidina-5(1,3)-benzenacyclotetradecaphane-12- |
| | yl)propanamide |
| 599 | 2,2-difluoro-3-hydroxy-N-((9S,12S)-5⁴-methyl-8,11,14-trioxo-9-phenethyl- |
| | 4-oxa-7,10-diaza-1(3,1)-piperidina-5(1,3)-benzenacyclotetradecaphane- |
| | 12-yl)propanamide |
| 600 | 2-(2-fluorophenyl)-N-((9S,12S)-5⁴-methyl-8,11,14-trioxo-9-(2-(pyridin-3- |
| | yl)ethyl)-4-oxa-7,10-diaza-1(3,1)-piperidina-5(1,3)- |
| | benzenacyclotetradecaphane-12-yl)acetamide |
| 601 | 2,2,2-trifluoro-N-((9S,12S)-5⁴-methyl-8,11,14-trioxo-9-phenethyl-4-oxa- |
| | 7,10-diaza-1(3,1)-piperidina-5(1,3)-benzenacyclotetradecaphane-12- |
| | yl)ethane-1-sulfonamide |
| 602 | N-((9S,12S)-9-(2,3-dihydro-1H-inden-2-yl)-5⁴-methyl-8,11,14-trioxo-4-oxa- |
| | 7,10-diaza-1(3,1)-piperidina-5(1,3)-benzenacyclotetradecaphane-12-yl)-2- |
| | (imidazo[2,1-b]thiazol-6-yl)acetamide |
| 603 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-3-((4-(pyridin- |
| | 4-yl)benzyl)amino)propanamide |
| 604 | N-(3-(((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)amino)-3- |
| | oxopropyl)-4-morpholinobenzamide |
| 605 | tert-butyl 3-(((9S,12S)-5⁴-methyl-8,11,14-trioxo-9-phenethyl-4-oxa-7,10- |
| | diaza-1(3,1)-piperidina-5(1,3)-benzenacyclotetradecaphane-12- |
| | yl)carbamoyl)-8-azabicyclo[3.2.1]octane-8-carboxylate |
| 606 | 3-(3-(4-methoxyphenyl)propoxy)-N-((9S,12S)-5⁴-methyl-8,11,14-trioxo-9- |
| | phenethyl-4-oxa-7,10-diaza-1(3,1)-piperidina-5(1,3)- |
| | benzenacyclotetradecaphane-12-yl)propanamide |
| 607 | 3-(3-(3-methoxyphenyl)propoxy)-N-((9S,12S)-5⁴-methyl-8,11,14-trioxo-9- |
| | phenethyl-4-oxa-7,10-diaza-1(3,1)-piperidina-5(1,3)- |
| | benzenacyclotetradecaphane-12-yl)propanamide |
| 608 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-3-(3- |
| | azaspiro[5.5]undecan-3-yl)propanamide |
| 609 | 2-methyl-N-((9S,12S)-5⁴-methyl-8,11,14-trioxo-9-phenethyl-4-oxa-7,10- |
| | diaza-1(3,1)-piperidina-5(1,3)-benzenacyclotetradecaphane-12-yl)-2- |
| | (quinolin-6-yl)propanamide |
| 610 | 3-(4-methoxyphenethoxy)-N-((9S,12S)-5⁴-methyl-8,11,14-trioxo-9- |
| | phenethyl-4-oxa-7,10-diaza-1(3,1)-piperidina-5(1,3)- |
| | benzenacyclotetradecaphane-12-yl)propanamide |
| 611 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-3-(3-oxa-9- |
| | azaspiro[5.5]undecan-9-yl)propanamide |
| 612 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-3-(3- |
| | phenylpropoxy)propanamide |
| 613 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-3-(8- |
| | azaspiro[4.5]decan-8-yl)propanamide |
| 614 | 3-(bicyclo[2.2.1]heptan-2-ylmethoxy)-N-((9S,12S)-5⁴-methyl-8,11,14- |
| | trioxo-9-phenethyl-4-oxa-7,10-diaza-1(3,1)-piperidina-5(1,3)- |
| | benzenacyclotetradecaphane-12-yl)propanamide |
| 615 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-3- |
| | (spiro[3.3]heptan-2-ylmethoxy)propanamide |
| 616 | N-(3-(((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)amino)-3- |
| | oxopropyl)spiro[3.3]heptane-2-carboxamide |
| 617 | N-(3-(((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)amino)-3- |
| | oxopropyl)bicyclo[2.2.1]heptane-2-carboxamide |
| 618 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-3- |
| | phenethoxypropanamide |
| 619 | 3-((2-methoxybenzyl)oxy)-N-((9S,12S)-5⁴-methyl-8,11,14-trioxo-9- |
| | phenethyl-4-oxa-7,10-diaza-1(3,1)-piperidina-5(1,3)- |
| | benzenacyclotetradecaphane-12-yl)propanamide |
| 620 | 3-(3-cyclopentylpropoxy)-N-((9S,12S)-5⁴-methyl-8,11,14-trioxo-9- |
| | phenethyl-4-oxa-7,10-diaza-1(3,1)-piperidina-5(1,3)- |
| | benzenacyclotetradecaphane-12-yl)propanamide |
| 621 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-3-(2-oxa-8- |
| | azaspiro[4.5]decan-8-yl)propanamide |
| 622 | 3-(4-methoxyphenoxy)-N-((9S,12S)-5⁴-methyl-8,11,14-trioxo-9-phenethyl- |
| | 4-oxa-7,10-diaza-1(3,1)-piperidina-5(1,3)-benzenacyclotetradecaphane- |
| | 12-yl)propanamide |
| 623 | 3-(2-cyclopentylethoxy)-N-((9S,12S)-5⁴-methyl-8,11,14-trioxo-9-phenethyl- |
| | 4-oxa-7,10-diaza-1(3,1)-piperidina-5(1,3)-benzenacyclotetradecaphane- |
| | 12-yl)propanamide |
| 624 | N-((9S,12S)-5⁴-methyl-8,11,16-trioxo-9-phenethyl-4,15-dioxa-7,10-diaza- |
| | 1(3,1)-piperidina-5(1,3)-benzenacyclohexadecaphane-12-yl)-2- |
| | phenylacetamide |
| 625 | 3-(cyclopentylmethoxy)-N-((9S,12S)-5⁴-methyl-8,11,14-trioxo-9-phenethyl- |
| | 4-oxa-7,10-diaza-1(3,1)-piperidina-5(1,3)-benzenacyclotetradecaphane- |
| | 12-yl)propanamide |
| 626 | 3-(3-cyclopropylpropoxy)-N-((9S,12S)-5⁴-methyl-8,11,14-trioxo-9- |
| | phenethyl-4-oxa-7,10-diaza-1(3,1)-piperidina-5(1,3)- |
| | benzenacyclotetradecaphane-12-yl)propanamide |
| 627 | 3-(2-cyclobutylethoxy)-N-((9S,12S)-5⁴-methyl-8,11,14-trioxo-9-phenethyl- |
| | 4-oxa-7,10-diaza-1(3,1)-piperidina-5(1,3)-benzenacyclotetradecaphane- |
| | 12-yl)propanamide |
| 628 | 3-(cyclobutylmethoxy)-N-((9S,12S)-5⁴-methyl-8,11,14-trioxo-9-phenethyl- |
| | 4-oxa-7,10-diaza-1(3,1)-piperidina-5(1,3)-benzenacyclotetradecaphane- |
| | 12-yl)propanamide |
| 629 | 3-(cyclopentyloxy)-N-((9S,12S)-5⁴-methyl-8,11,14-trioxo-9-phenethyl-4- |
| | oxa-7,10-diaza-1(3,1)-piperidina-5(1,3)-benzenacyclotetradecaphane-12- |
| | yl)propanamide |
| 630 | 3-(2-cyclopropylethoxy)-N-((9S,12S)-5⁴-methyl-8,11,14-trioxo-9- |
| | phenethyl-4-oxa-7,10-diaza-1(3,1)-piperidina-5(1,3)- |
| | benzenacyclotetradecaphane-12-yl)propanamide |
| 631 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12- |
| | yl)spiro[3.3]heptane-2-carboxamide |
| 632 | cyclohexyl ((9S,12S)-5⁴-methyl-8,11,14-trioxo-9-phenethyl-4-oxa-7,10- |
| | diaza-1(3,1)-piperidina-5(1,3)-benzenacyclotetradecaphane-12- |
| | yl)carbamate |
| 633 | 3-fluoro-N-((9S,12S)-5⁴-methyl-8,11,14-trioxo-9-phenethyl-4-oxa-7,10- |
| | diaza-1(3,1)-piperidina-5(1,3)-benzenacyclotetradecaphane-12- |
| | yl)bicyclo[1.1.1]pentane-1-carboxamide |
| 634 | (2S)-2-(4-aminophenyl)-N-((9S,12S)-5⁴-methyl-8,11,14-trioxo-9-phenethyl- |
| | 4-oxa-7,10-diaza-1(3,1)-piperidina-5(1,3)-benzenacyclotetradecaphane- |
| | 12-yl)propanamide |
| 635 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-2-(oxetan-3- |
| | yl)acetamide |
| 636 | 1-(2-fluorophenyl)-N-((9S,12S)-5⁴-methyl-8,11,14-trioxo-9-phenethyl-4- |
| | oxa-7,10-diaza-1(3,1)-piperidina-5(1,3)-benzenacyclotetradecaphane-12- |
| | yl)methanesulfonamide |
| 637 | 3-(3-(2-methoxyphenyl)propoxy)-N-((9S,12S)-5⁴-methyl-8,11,14-trioxo-9- |
| | phenethyl-4-oxa-7,10-diaza-1(3,1)-piperidina-5(1,3)- |
| | benzenacyclotetradecaphane-12-yl)propanamide |
| 638 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-3-(7- |
| | azaspiro[3.5]nonan-7-yl)propanamide |
| 639 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-3-(6- |
| | azaspiro[3.4]octan-6-yl)propanamide |
| 640 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-3-(2-oxa-7- |
| | azaspiro[4.4]nonan-7-yl)propanamide |
| 641 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-3-(2-oxa-7- |
| | azaspiro[3.5]nonan-7-yl)propanamide |
| 642 | 3-(((1S,2S,4R)-bicyclo[2.2.1]heptan-2-yl)oxy)-N-((9S,12S)-5⁴-methyl- |
| | 8,11,14-trioxo-9-phenethyl-4-oxa-7,10-diaza-1(3,1)-piperidina-5(1,3)- |
| | benzenacyclotetradecaphane-12-yl)propanamide |
| 643 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12- |
| | yl)cyclohexanecarboxamide |
| 644 | 3-hydroxy-2,2-dimethyl-N-((13S,9S,12S)-5⁴-methyl-8,11,14-trioxo-9- |
| | phenethyl-4-oxa-7,10-diaza-1(3,1)-piperidina-5(1,3)- |
| | benzenacyclotetradecaphane-12-yl)propanamide |
| 645 | 2-(4-amino-2-methylphenyl)-N-((9S,12S)-5⁴-methyl-8,11,14-trioxo-9-(2- |
| | (pyridin-3-yl)ethyl)-4-oxa-7,10-diaza-1(3,1)-piperidina-5(1,3)- |
| | benzenacyclotetradecaphane-12-yl)acetamide |
| 646 | 3,3,3-trifluoro-2,2-dimethyl-N-((9S,12S)-5⁴-methyl-8,11,14-trioxo-9- |
| | phenethyl-4-oxa-7,10-diaza-1(3,1)-piperidina-5(1,3)- |
| | benzenacyclotetradecaphane-12-yl)propanamide |
| 647 | 3-(benzylamino)-N-((9S,12S)-5⁴-methyl-8,11,14-trioxo-9-phenethyl-4-oxa- |
| | 7,10-diaza-1(3,1)-piperidina-5(1,3)-benzenacyclotetradecaphane-12- |
| | yl)propanamide |
| 648 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-3-(2-oxa-6- |
| | azaspiro[3.4]octan-6-yl)propanamide |
| 649 | 3-(cyclopropylamino)-N-((9S,12S)-5⁴-methyl-8,11,14-trioxo-9-phenethyl-4- |
| | oxa-7,10-diaza-1(3,1)-piperidina-5(1,3)-benzenacyclotetradecaphane-12- |
| | yl)propanamide |
| 650 | 3-fluoro-2,2-dimethyl-N-((9S,12S)-5⁴-methyl-8,11,14-trioxo-9-phenethyl-4- |
| | oxa-7,10-diaza-1(3,1)-piperidina-5(1,3)-benzenacyclotetradecaphane-12- |
| | yl)propanamide |
| 651 | 3-isobutoxy-N-(3-(((9S,12S)-5⁴-methyl-8,11,14-trioxo-9-phenethyl-4-oxa- |
| | 7,10-diaza-1(3,1)-piperidina-5(1,3)-benzenacyclotetradecaphane-12- |
| | yl)amino)-3-oxopropyl)benzamide |
| 652 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-3-(2- |
| | azaspiro[4.4]nonan-2-yl)propanamide |
| 653 | N-((9S,12S)-5⁴-methyl-8,11,16-trioxo-9-phenethyl-4-oxa-7,10,15-triaza- |
| | 1(3,1)-piperidina-5(1,3)-benzenacyclohexadecaphane-12-yl)-2- |
| | phenylacetamide |
| 654 | 2-(4-amino-2-methylphenyl)-N-((9S,12S)-5⁴-methyl-8,11,14-trioxo-9-(2- |
| | (pyridin-4-yl)ethyl)-4-oxa-7,10-diaza-1(3,1)-piperidina-5(1,3)- |
| | benzenacyclotetradecaphane-12-yl)acetamide |
| 655 | 3-hydroxy-N-((9S,12S)-5⁴-methyl-8,11,14-trioxo-9-phenethyl-4-oxa-7,10- |
| | diaza-1(3,1)-piperidina-5(1,3)-benzenacyclotetradecaphane-12- |
| | yl)bicyclo[1.1.1]pentane-1-carboxamide |
| 656 | 4-cyclohexyl-N-(3-(((9S,12S)-5⁴-methyl-8,11,14-trioxo-9-phenethyl-4-oxa- |
| | 7,10-diaza-1(3,1)-piperidina-5(1,3)-benzenacyclotetradecaphane-12- |
| | yl)amino)-3-oxopropyl)benzamide |
| 657 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-3- |
| | (spiro[3.3]heptan-2-ylamino)propanamide |
| 658 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-3-(7-oxa-2- |
| | azaspiro[3.5]nonan-2-yl)propanamide |
| 659 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-3-(2- |
| | azaspiro[3.3]heptan-2-yl)propanamide |
| 660 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-3-(2-oxa-6- |
| | azaspiro[3.3]heptan-6-yl)propanamide |
| 661 | N-((9S,12S)-5⁴-fluoro-8,11,14-trioxo-9-phenethyl-4-oxa-7,10-diaza-1(3,1)- |
| | piperidina-5(1,3)-benzenacyclotetradecaphane-12-yl)-2-phenylacetamide |
| 662 | 3-amino-N-((9S,12S)-5⁴-methyl-8,11,14-trioxo-9-phenethyl-4-oxa-7,10- |
| | diaza-1(3,1)-piperidina-5(1,3)-benzenacyclotetradecaphane-12- |
| | yl)bicyclo[1.1.1]pentane-1-carboxamide |
| 663 | 2-(azetidin-1-yl)-N-((9S,12S)-5⁴-methyl-8,11,14-trioxo-9-phenethyl-4-oxa- |
| | 7,10-diaza-1(3,1)-piperidina-5(1,3)-benzenacyclotetradecaphane-12- |
| | yl)acetamide |
| 664 | 1-(hydroxymethyl)-N-((9S,12S)-5⁴-methyl-8,11,14-trioxo-9-phenethyl-4- |
| | oxa-7,10-diaza-1(3,1)-piperidina-5(1,3)-benzenacyclotetradecaphane-12- |
| | yl)cyclopropane-1-carboxamide |
| 665 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-3-((3- |
| | morpholinobenzyl)amino)propanamide |
| 666 | 5-amino-3,3-dimethyl-N-((9S,12S)-5⁴-methyl-8,11,14-trioxo-9-phenethyl-4- |
| | oxa-7,10-diaza-1(3,1)-piperidina-5(1,3)-benzenacyclotetradecaphane-12- |
| | yl)indoline-1-carboxamide |
| 667 | 6-amino-N-((9S,12S)-5⁴-methyl-8,11,14-trioxo-9-phenethyl-4-oxa-7,10- |
| | diaza-1(3,1)-piperidina-5(1,3)-benzenacyclotetradecaphane-12-yl)-3,4- |
| | dihydroquinoline-1(2H)-carboxamide |
| 668 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-3-(pyrrolidin- |
| | 1-yl)propanamide |
| 669 | 3-hydroxy-3-methyl-N-((9S,12S)-5⁴-methyl-8,11,14-trioxo-9-phenethyl-4- |
| | oxa-7,10-diaza-1(3,1)-piperidina-5(1,3)-benzenacyclotetradecaphane-12- |
| | yl)azetidine-1-carboxamide |
| 670 | 1-(hydroxymethyl)-N-((9S,12S)-5⁴-methyl-8,11,14-trioxo-9-phenethyl-4- |
| | oxa-7,10-diaza-1(3,1)-piperidina-5(1,3)-benzenacyclotetradecaphane-12- |
| | yl)cyclobutane-1-carboxamide |
| 671 | 4-(hydroxymethyl)-N-((9S,12S)-5⁴-methyl-8,11,14-trioxo-9-phenethyl-4- |
| | oxa-7,10-diaza-1(3,1)-piperidina-5(1,3)-benzenacyclotetradecaphane-12- |
| | yl)tetrahydro-2H-pyran-4-carboxamide |
| 672 | 3-((4-(cyclohexylmethoxy)phenyl)sulfonamido)-N-((9S,12S)-5⁴-methyl- |
| | 8,11,14-trioxo-9-phenethyl-4-oxa-7,10-diaza-1(3,1)-piperidina-5(1,3)- |
| | benzenacyclotetradecaphane-12-yl)propanamide |
| 673 | 3-(2-azabicyclo[2.2.1]heptan-2-yl)-N-((9S,12S)-5⁴-methyl-8,11,14-trioxo-9- |
| | phenethyl-4-oxa-7,10-diaza-1(3,1)-piperidina-5(1,3)- |
| | benzenacyclotetradecaphane-12-yl)propanamide |
| 674 | 3-((cyclobutylmethyl)amino)-N-((9S,12S)-5⁴-methyl-8,11,14-trioxo-9- |
| | phenethyl-4-oxa-7,10-diaza-1(3,1)-piperidina-5(1,3)- |
| | benzenacyclotetradecaphane-12-yl)propanamide |
| 675 | 3-(cyclopentylamino)-N-((9S,12S)-5⁴-methyl-8,11,14-trioxo-9-phenethyl-4- |
| | oxa-7,10-diaza-1(3,1)-piperidina-5(1,3)-benzenacyclotetradecaphane-12- |
| | yl)propanamide |
| 676 | 4-hydroxy-4-methyl-N-((9S,12S)-5⁴-methyl-8,11,14-trioxo-9-phenethyl-4- |
| | oxa-7,10-diaza-1(3,1)-piperidina-5(1,3)-benzenacyclotetradecaphane-12- |
| | yl)piperidine-1-carboxamide |
| 677 | 3-((cyclopentylmethyl)amino)-N-((9S,12S)-5⁴-methyl-8,11,14-trioxo-9- |
| | phenethyl-4-oxa-7,10-diaza-1(3,1)-piperidina-5(1,3)- |
| | benzenacyclotetradecaphane-12-yl)propanamide |
| 678 | 2-(4-amino-2-methylphenyl)-N-((12S)-5⁴-methyl-8,11,14-trioxo-9-(2- |
| | (pyridin-4-yl)ethyl)-4-oxa-7,10-diaza-1(3,1)-piperidina-5(1,3)- |
| | benzenacyclotetradecaphane-12-yl)acetamide |
| 679 | 3-((2-cyclopropylethyl)amino)-N-((9S,12S)-5⁴-methyl-8,11,14-trioxo-9- |
| | phenethyl-4-oxa-7,10-diaza-1(3,1)-piperidina-5(1,3)- |
| | benzenacyclotetradecaphane-12-yl)propanamide |
| 680 | 3-(cyclobutylamino)-N-((9S,12S)-5⁴-methyl-8,11,14-trioxo-9-phenethyl-4- |
| | oxa-7,10-diaza-1(3,1)-piperidina-5(1,3)-benzenacyclotetradecaphane-12- |
| | yl)propanamide |
| 681 | 4-hydroxy-N-((9S,12S)-5⁴-methyl-8,11,14-trioxo-9-phenethyl-4-oxa-7,10- |
| | diaza-1(3,1)-piperidina-5(1,3)-benzenacyclotetradecaphane-12- |
| | yl)piperidine-1-carboxamide |
| 682 | 3-amino-2,2-dimethyl-N-((9S,12S)-5⁴-methyl-8,11,14-trioxo-9-phenethyl-4- |
| | oxa-7,10-diaza-1(3,1)-piperidina-5(1,3)-benzenacyclotetradecaphane-12- |
| | yl)propanamide |
| 683 | 3-hydroxy-2,2-dimethyl-N-((9S,12S)-5⁴-methyl-8,11,14-trioxo-9-(2-(pyridin- |
| | 3-yl)ethyl)-4-oxa-7,10-diaza-1(3,1)-piperidina-5(1,3)- |
| | benzenacyclotetradecaphane-12-yl)propanamide |
| 684 | N-((9S,12S)-5⁴-fluoro-8,11,14-trioxo-9-phenethyl-4-oxa-7,10-diaza-1(3,1)- |
| | piperidina-5(1,3)-benzenacyclotetradecaphane-12-yl)-3-hydroxy-2,2- |
| | dimethylpropanamide |
| 685 | N-((9S,12S)-9-(2,3-dihydro-1H-inden-2-yl)-5⁴-methyl-8,11,14-trioxo-4-oxa- |
| | 7,10-diaza-1(3,1)-piperidina-5(1,3)-benzenacyclotetradecaphane-12- |
| | yl)acetamide |
| 686 | N-((9S,12S)-9-(but-3-yn-1-yl)-5⁴-methyl-8,11,14-trioxo-4-oxa-7,10-diaza- |
| | 1(3,1)-piperidina-5(1,3)-benzenacyclotetradecaphane-12-yl)-2- |
| | (imidazo[2,1-b]thiazol-6-yl)acetamide |
| 687 | 5-amino-N-((9S,12S)-5⁴-methyl-8,11,14-trioxo-9-phenethyl-4-oxa-7,10- |
| | diaza-1(3,1)-piperidina-5(1,3)-benzenacyclotetradecaphane-12- |
| | yl)isoindoline-2-carboxamide |
| 688 | 4-ethyl-N-((9S,12S)-5⁴-methyl-8,11,14-trioxo-9-phenethyl-4-oxa-7,10- |
| | diaza-1(3,1)-piperidina-5(1,3)-benzenacyclotetradecaphane-12- |
| | yl)piperazine-1-carboxamide |
| 689 | 1-(hydroxymethyl)-N-((9S,12S)-5⁴-methyl-8,11,14-trioxo-9-phenethyl-4- |
| | oxa-7,10-diaza-1(3,1)-piperidina-5(1,3)-benzenacyclotetradecaphane-12- |
| | yl)cyclopentane-1-carboxamide |
| 690 | (9S,12S)-12-((3-hydroxy-2,2-dimethylpropyl)amino)-5⁴-methyl-9- |
| | phenethyl-4-oxa-7,10-diaza-1(3,1)-piperidina-5(1,3)- |
| | benzenacyclotetradecaphane-8,11,14-trione |
| 691 | 3-hydroxy-N-((9S,12S)-5⁴-methyl-8,11,14-trioxo-9-phenethyl-4-oxa-7,10- |
| | diaza-1(3,1)-piperidina-5(1,3)-benzenacyclotetradecaphane-12- |
| | yl)azetidine-1-carboxamide |
| 692 | 3-(3-cyclopentylpropoxy)-N-((9S,12S)-5⁴-methyl-8,11,14-trioxo-9- |
| | phenethyl-4-oxa-7,10-diaza-1(3,1)-piperidina-5(1,3)- |
| | benzenacyclotetradecaphane-12-yl)propanamide |
| 693 | 3-((2-cyclobutylethyl)amino)-N-((9S,12S)-5⁴-methyl-8,11,14-trioxo-9- |
| | phenethyl-4-oxa-7,10-diaza-1(3,1)-piperidina-5(1,3)- |
| | benzenacyclotetradecaphane-12-yl)propanamide |
| 694 | 3-((cyclopropylmethyl)amino)-N-((9S,12S)-5⁴-methyl-8,11,14-trioxo-9- |
| | phenethyl-4-oxa-7,10-diaza-1(3,1)-piperidina-5(1,3)- |
| | benzenacyclotetradecaphane-12-yl)propanamide |
| 695 | 3-(((1R,2R,4S)-bicyclo[2.2.1]heptan-2-yl)amino)-N-((9S,12S)-5⁴-methyl- |
| | 8,11,14-trioxo-9-phenethyl-4-oxa-7,10-diaza-1(3,1)-piperidina-5(1,3)- |
| | benzenacyclotetradecaphane-12-yl)propanamide |
| 696 | N-((9S,12S)-54,15-dimethyl-8,11,16-trioxo-9-phenethyl-4-oxa-7,10,15- |
| | triaza-1(3,1)-piperidina-5(1,3)-benzenacyclohexadecaphane-12-yl)-2- |
| | phenylacetamide |
| 697 | 2-(1-acetylazetidin-3-yl)-N-((9S,12S)-5⁴-methyl-8,11,14-trioxo-9- |
| | phenethyl-4-oxa-7,10-diaza-1(3,1)-piperidina-5(1,3)- |
| | benzenacyclotetradecaphane-12-yl)acetamide |
| 698 | 3-((2-(bicyclo[2.2.1]heptan-2-yl)ethyl)amino)-N-((9S,12S)-5⁴-methyl- |
| | 8,11,14-trioxo-9-phenethyl-4-oxa-7,10-diaza-1(3,1)-piperidina-5(1,3)- |
| | benzenacyclotetradecaphane-12-yl)propanamide |
| 699 | 3-((4-(4-benzylpiperazin-1-yl)benzyl)amino)-N-((9S,12S)-5⁴-methyl- |
| | 8,11,14-trioxo-9-phenethyl-4-oxa-7,10-diaza-1(3,1)-piperidina-5(1,3)- |
| | benzenacyclotetradecaphane-12-yl)propanamide |
| 700 | 3-((2-cyclopentylethyl)amino)-N-((9S,12S)-5⁴-methyl-8,11,14-trioxo-9- |
| | phenethyl-4-oxa-7,10-diaza-1(3,1)-piperidina-5(1,3)- |
| | benzenacyclotetradecaphane-12-yl)propanamide |
| 701 | N-(3-(((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)amino)-3- |
| | oxopropyl)-7-oxabicyclo[2.2.1]heptane-2-carboxamide |
| 702 | 3-(azetidin-1-yl)-N-((9S,12S)-5⁴-methyl-8,11,14-trioxo-9-phenethyl-4-oxa- |
| | 7,10-diaza-1(3,1)-piperidina-5(1,3)-benzenacyclotetradecaphane-12- |
| | yl)propanamide |
| 703 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-2,3-dihydro- |
| | 1H-pyrrolo[3,2-c]pyridine-1-carboxamide |
| 704 | 5-amino-N-((9S,12S)-5⁴-methyl-8,11,14-trioxo-9-phenethyl-4-oxa-7,10- |
| | diaza-1(3,1)-piperidina-5(1,3)-benzenacyclotetradecaphane-12- |
| | yl)indoline-1-carboxamide |
| 705 | 5-hydroxy-N-((9S,12S)-5⁴-methyl-8,11,14-trioxo-9-phenethyl-4-oxa-7,10- |
| | diaza-1(3,1)-piperidina-5(1,3)-benzenacyclotetradecaphane-12- |
| | yl)indoline-1-carboxamide |
| 706 | 3-((4-(cyclohexylmethoxy)benzyl)amino)-N-((9S,12S)-5⁴-methyl-8,11,14- |
| | trioxo-9-phenethyl-4-oxa-7,10-diaza-1(3,1)-piperidina-5(1,3)- |
| | benzenacyclotetradecaphane-12-yl)propanamide |
| 707 | 2-(4-acetylphenyl)-N-((11S,14S)-14-methyl-8,12,15-trioxo-1⁴-phenethyl-2- |
| | oxa-7,13,16-triaza-1(1,3)-benzenacycloheptadecaphane-11-yl)acetamide |
| 708 | N-((9S,12S)-9-(4-aminophenethyl)-5⁴-methyl-8,11,14-trioxo-4-oxa-7,10- |
| | diaza-1(3,1)-piperidina-5(1,3)-benzenacyclotetradecaphane-12-yl)-2- |
| | (imidazo[2,1-b]thiazol-6-yl)acetamide |
| 709 | (9S,12S)-5⁴-methyl-9-phenethyl-12-((2,2,2-trifluoroethyl)amino)-4-oxa- |
| | 7,10-diaza-1(3,1)-piperidina-5(1,3)-benzenacyclotetradecaphane-8,11,14- |
| | trione |
| 710 | 3-((2-methyl-2-azaspiro[3.3]heptan-6-yl)oxy)-N-((9S,12S)-5⁴-methyl- |
| | 8,11,14-trioxo-9-phenethyl-4-oxa-7,10-diaza-1(3,1)-piperidina-5(1,3)- |
| | benzenacyclotetradecaphane-12-yl)propanamide |
| 711 | (9S,12S)-12-(bicyclo[2.2.1]hept-5-en-2-ylamino)-5⁴-methyl-9-phenethyl-4- |
| | oxa-7,10-diaza-1(3,1)-piperidina-5(1,3)-benzenacyclotetradecaphane- |
| | 8,11,14-trione |
| 712 | (9S,12S)-12-(bicyclo[2.2.1]heptan-2-ylamino)-5⁴-methyl-9-phenethyl-4- |
| | oxa-7,10-diaza-1(3,1)-piperidina-5(1,3)-benzenacyclotetradecaphane- |
| | 8,11,14-trione |
| 713 | 8-methyl-N-((9S,12S)-5⁴-methyl-8,11,14-trioxo-9-phenethyl-4-oxa-7,10- |
| | diaza-1(3,1)-piperidina-5(1,3)-benzenacyclotetradecaphane-12-yl)-8- |
| | azabicyclo[3.2.1]octane-3-carboxamide |
| 714 | 2-methyl-N-((9S,12S)-5⁴-methyl-8,11,14-trioxo-9-phenethyl-4-oxa-7,10- |
| | diaza-1(3,1)-piperidina-5(1,3)-benzenacyclotetradecaphane-12-yl)-2- |
| | azaspiro[3.3]heptane-6-carboxamide |
| 715 | 3-methyl-N-((9S,12S)-5⁴-methyl-8,11,14-trioxo-9-phenethyl-4-oxa-7,10- |
| | diaza-1(3,1)-piperidina-5(1,3)-benzenacyclotetradecaphane-12- |
| | yl)azetidine-3-carboxamide |
| 716 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)piperazine-1- |
| | carboxamide |
| 717 | (9S,12S)-12-(cyclobutylamino)-5⁴-methyl-9-phenethyl-4-oxa-7,10-diaza- |
| | 1(3,1)-piperidina-5(1,3)-benzenacyclotetradecaphane-8,11,14-trione |
| 718 | 2-(4-amino-2-methylphenyl)-N-((12S,15S)-1⁴-methyl-8,13,16-trioxo-15- |
| | phenethyl-2-oxa-7,14,17-triaza-1(1,3)-benzenacyclooctadecaphane-12- |
| | yl)acetamide |
| 719 | (3S)-3-amino-4-(((9S,12S)-5⁴-methyl-8,11,14-trioxo-9-phenethyl-4-oxa- |
| | 7,10-diaza-1(3,1)-piperidina-5(1,3)-benzenacyclotetradecaphane-12- |
| | yl)amino)-4-oxobutanoic acid |
| 720 | 3-((2-oxaspiro[3.3]heptan-6-yl)amino)-N-((9S,12S)-5⁴-methyl-8,11,14- |
| | trioxo-9-phenethyl-4-oxa-7,10-diaza-1(3,1)-piperidina-5(1,3)- |
| | benzenacyclotetradecaphane-12-yl)propanamide |
| 721 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-2- |
| | azaspiro[3.3]heptane-6-carboxamide |
| 722 | 3-hydroxy-2,2-dimethyl-N-((12S,15S)-1⁴-methyl-8,13,16-trioxo-15- |
| | phenethyl-2-oxa-7,14,17-triaza-1(1,3)-benzenacyclooctadecaphane-12- |
| | yl)propanamide |
| 723 | N-((9S,12S)-5⁴-methyl-8,11,14-trioxo-9-phenethyl-4-oxa-7,10-diaza- |
| | 1(3,1)-piperidina-5(1,3)-benzenacyclotetradecaphane-12-yl)-8- |
| | azabicyclo[3.2.1]octane-3-carboxamide |
| 724 | (4S)-4-amino-5-(((9S,12S)-5⁴-methyl-8,11,14-trioxo-9-phenethyl-4-oxa- |
| | 7,10-diaza-1(3,1)-piperidina-5(1,3)-benzenacyclotetradecaphane-12- |
| | yl)amino)-5-oxopentanoic acid |
| 725 | 3-((2-azaspiro[3.3]heptan-6-yl)oxy)-N-((9S,12S)-5⁴-methyl-8,11,14-trioxo- |
| | 9-phenethyl-4-oxa-7,10-diaza-1(3,1)-piperidina-5(1,3)- |
| | benzenacyclotetradecaphane-12-yl)propanamide |
| 726 | 3-hydroxy-2,2-dimethyl-N-((11S,14S)-1⁴-methyl-8,12,15-trioxo-14- |
| | phenethyl-2-oxa-7,13,16-triaza-1(1,3)-benzenacycloheptadecaphane-11- |
| | yl)propanamide |
| 727 | (9S,12S)-12-((2-amino-2-methylpropyl)amino)-5⁴-methyl-9-phenethyl-4- |
| | oxa-7,10-diaza-1(3,1)-piperidina-5(1,3)-benzenacyclotetradecaphane- |
| | 8,11,14-trione |
| 728 | (9S,12S)-5⁴-methyl-12-(2-oxopyrrolidin-1-yl)-9-phenethyl-4-oxa-7,10- |
| | diaza-1(3,1)-piperidina-5(1,3)-benzenacyclotetradecaphane-8,11,14-trione |
| 729 | 4-fluoro-N-((12S)-5⁴-methyl-8,11,14-trioxo-9-(2-(pyridin-4-yl)ethyl)-4-oxa- |
| | 7,10-diaza-1(3,1)-piperidina-5(1,3)-benzenacyclotetradecaphane-12- |
| | yl)piperidine-4-carboxamide |
| 730 | 4-(hydroxymethyl)-N-((9S,12S)-5⁴-methyl-8,11,14-trioxo-9-phenethyl-4- |
| | oxa-7,10-diaza-1(3,1)-piperidina-5(1,3)-benzenacyclotetradecaphane-12- |
| | yl)piperidine-4-carboxamide |
| 731 | (9S,12S)-5⁴-methyl-9-phenethyl-12-(pyrrolidin-1-yl)-4-oxa-7,10-diaza- |
| | 1(3,1)-piperidina-5(1,3)-benzenacyclotetradecaphane-8,11,14-trione |
| 732 | 3-hydroxy-2,2-dimethyl-N-((10S,13S)-1⁴-methyl-7,11,14-trioxo-13- |
| | phenethyl-2-oxa-6,12,15-triaza-1(1,3)-benzenacyclohexadecaphane-10- |
| | yl)propanamide |
| 733 | (9S,12S)-12-(4,4-dimethyl-2-oxoimidazolidin-1-yl)-5⁴-methyl-9-phenethyl- |
| | 4-oxa-7,10-diaza-1(3,1)-piperidina-5(1,3)-benzenacyclotetradecaphane- |
| | 8,11,14-trione |
| 734 | (9S,12S)-5⁴-methyl-12-(2-oxopiperidin-1-yl)-9-phenethyl-4-oxa-7,10-diaza- |
| | 1(3,1)-piperidina-5(1,3)-benzenacyclotetradecaphane-8,11,14-trione |
| 735 | 3-hydroxy-2,2-dimethyl-N-((10S,13S)-1⁴-methyl-8,11,14-trioxo-13- |
| | phenethyl-2-oxa-7,12,15-triaza-1(1,3)-benzenacyclohexadecaphane-10- |
| | yl)propanamide |
| 736 | 3-hydroxy-2,2-dimethyl-N-((9S,12S)-1⁴-methyl-7,10,13-trioxo-12- |
| | phenethyl-2-oxa-6,11,14-triaza-1(1,3)-benzenacyclopentadecaphane-9- |
| | yl)propanamide |
| 737 | 2-(2-fluorophenyl)-N-((9S,12S)-5⁴-methyl-8,11,14-trioxo-9-(2-(pyridin-4- |
| | yl)ethyl)-4-oxa-7,10-diaza-1(3,1)-piperidina-5(1,3)- |
| | benzenacyclotetradecaphane-12-yl)acetamide |
| 738 | (2R)-2-(4-aminophenyl)-N-((9S,12S)-5⁴-methyl-8,11,14-trioxo-9- |
| | phenethyl-4-oxa-7,10-diaza-1(3,1)-piperidina-5(1,3)- |
| | benzenacyclotetradecaphane-12-yl)propanamide |
| 739 | N-((9S,12S)-9-(2-((1S,4S)-2-oxa-5-azabicyclo[2.2.1]heptan-5-yl)ethyl)-5⁴- |
| | methyl-8,11,14-trioxo-4-oxa-7,10-diaza-1(3,1)-piperidina-5(1,3)- |
| | benzenacyclotetradecaphane-12-yl)-2-(imidazo[2,1-b]thiazol-6- |
| | yl)acetamide |
| 740 | (9S,12S)-5⁴-methyl-12-(4-methyl-1H-1,2,3-triazol-1-yl)-9-phenethyl-4-oxa- |
| | 7,10-diaza-1(3,1)-piperidina-5(1,3)-benzenacyclotetradecaphane-8,11,14- |
| | trione |
| 741 | 3-((4-methoxybenzyl)oxy)-N-((9S,12S)-5⁴-methyl-8,11,14-trioxo-9- |
| | phenethyl-4-oxa-7,10-diaza-1(3,1)-piperidina-5(1,3)- |
| | benzenacyclotetradecaphane-12-yl)propanamide |

or an enantiomer, a stereoisomeric form, a mixture of enantiomers, a diastereomer, a mixture of diastereomers, a hydrate, a solvate, an acid salt form, a tautomer, a racemate of the above mentioned compounds, or a pharmaceutically acceptable salt thereof.

11. A pharmaceutical composition comprising at least one compound according to claim 1 as an active ingredient, together with at least one pharmaceutically acceptable carrier, excipient and/or diluent.

12. (canceled)

13. A method for prophylaxis or treatment of a disease associated with or caused by proteasome or immunoproteasome, selected from a cancer, an infectious disease, an inflammatory disease, autoimmune disease, and transplant rejection in a mammal, comprising administering to the mammal at least one compound according to claim 1 or a pharmaceutically acceptable salt thereof, effective to prevent or treat the disease associated with or caused by proteasome or immunoproteasome, selected from a cancer, an infectious disease, an inflammatory disease, autoimmune disease, and transplant rejection.

14. The method according to claim 13, wherein the cancer is selected from the group consisting of: adenocarcinoma, choroidal melanoma, acute leukemia, acoustic neurinoma, ampullary carcinoma, anal carcinoma, astrocytoma, basal cell carcinoma, pancreatic cancer, desmoid tumour, bladder cancer, bronchial carcinoma, non-small cell lung cancer (NSCLC), breast cancer, Burkitt's lymphoma, corpus cancer, CUP-syndrome (carcinoma of unknown primary), colorectal cancer, small intestine cancer, small intestinal tumours, ovarian cancer, endometrial carcinoma, ependymoma, epithelial cancer types, Ewing's tumours, gastrointestinal tumours, gastric cancer, gallbladder cancer, gall bladder carcinomas, uterine cancer, cervical cancer, glioblastomas, gynecologic tumours, ear, nose and throat tumours, hematologic neoplasias, hairy cell leukemia, urethral cancer, skin cancer, skin testis cancer, brain tumours (gliomas), brain metastases, testicle cancer, hypophysis tumour, carcinoids, Kaposi's sarcoma, laryngeal cancer, germ cell tumour, bone cancer, colorectal carcinoma, head and neck tumours (tumours of the ear, nose and throat area), colon carcinoma, craniopharyngiomas, oral cancer (cancer in the mouth area and on lips), cancer of the central nervous system, liver cancer, liver metastases, leukemia, eyelid tumor, lung cancer, lymph node cancer (Hodgkin's/Non-Hodgkin's), lymphomas, stomach cancer, malignant melanoma, malignant neoplasia, malignant tumours gastrointestinal tract, breast carcinoma, rectal cancer, medulloblastomas, melanoma, meningiomas, Hodgkin's disease, mycosis fungoides, nasal cancer, neurinoma, neuroblastoma, kidney cancer, renal cell carcinomas, non-Hodgkin's lymphomas, oligodendroglioma, esophageal carcinoma, osteolytic carcinomas and osteoplastic carcinomas, osteosarcomas, ovarial carcinoma, pancreatic carcinoma, penile cancer, plasmocytoma, squamous cell carcinoma of the head and neck (SCCHN), prostate cancer, pharyngeal cancer, rectal carcinoma, retinoblastoma, vaginal cancer, thyroid carcinoma, Schneeberger disease, esophageal cancer, spinalioms, T-cell lymphoma (mycosis fungoides), thymoma, tube carcinoma, eye tumours, urethral cancer, urologic tumours, urothelial carcinoma, vulva cancer, wart appearance, soft tissue tumours, soft tissue sarcoma, Wilm's tumour, cervical carcinoma, tongue cancer, astrocytomas, bronchial cancer, laryngeal cancer, malignant melanoma, oesophageal cancer, cholangiocarcinoma, and renal cell cancer.

15. The method according to claim 13, wherein the cancer is leukemia, multiple myeloma, mantle-cell lymphoma (MCL), breast cancer, colorectal cancer, non-small cell lung cancer, or ovarian cancer.

16. The method according to claim 14, wherein the cancer is multiple myeloma.

17. The method according to claim 16, in combination with a thalidomide or a derivative thereof.

18. The method according to claim 17, wherein the derivative of thalidomide is selected from lenalidomide, pomalidomide, avadomide, iberdomide, and CC-885.

19. The method according to claim 13, wherein the infectious disease is selected from the group consisting of: HIV, Echinococcosis, Amebiasis (Entamoeba histolytica Infection), Angiostrongylus Infection, Anisakiasis, Anthrax, Babesiosis (Babesia Infection), Balantidium Infection (Balantidiasis), Baylisascaris Infection (Raccoon Roundworm), Bilharzia (Schistosomiasis), Blastocystis hominis Infection (Blastomycosis), Borreliosis, Botulism, Brainerd Diarrhea, Brucellosis, BSE (Bovine Spongiform Encephalopathy), Candidiasis, Capillariasis (Capillaria Infection), CFS (Chronic Fatigue Syndrome), Chagas Disease (American Trypanosomiasis), Chickenpox (Varicella-Zoster virus), Chlamydia pneumoniae Infection, Cholera, CJD (Creutzfeldt-Jakob Disease), Clonorchiasis (Clonorchis Infection), CLM (Cutaneous Larva Migrans, Hookworm Infection), Coccidioidomycosis, Conjunctivitis, Coxsackievirus A16 (Hand, Foot and Mouth Disease), Cryptococcosis, Cryptosporidium Infection (Cryptosporidiosis), Culex mosquito (Vector of West Nile Virus), Cyclosporiasis (Cyclospora Infection), Cysticercosis (Neurocysticercosis), Cytomegalovirus Infection, Dengue/Dengue Fever, Dipylidium Infection (Dog and Cat Flea Tapeworm), Ebola Virus Hemorrhagic Fever, Echinococcosis (Alveolar Hydatid Disease), Encephalitis, Entamoeba coli Infection, Entamoeba dispar Infection, Entamoeba hartmanni Infection, Entamoeba histolytica Infection (Amebiasis), Entamoeba polecki Infection, Enterobiasis (Pinworm Infection), Enterovirus Infection (non-polio), Epstein-Barr Virus Infection, Escherichia coli Infection, Foodborne Infection, Foot and mouth Disease, Fungal Dermatitis, Gastroenteritis, Group A streptococcal Disease, Group B streptococcal Disease, Hansen's Disease (Leprosy), Hantavirus Pulmonary Syndrome, Head Lice Infestation (Pediculosis), Helicobacter pylori Infection, Hematologic Disease, Hendra Virus Infection, Hepatitis (HCV, HBV), Herpes Zoster (Shingles), Human Ehrlichiosis, Human Parainfluenza Virus Infection, Influenza, Isosporiasis (Isospora Infection), Lassa Fever, Leishmaniasis, Kala-azar (Kala-azar, Leishmania Infection), Leprosy, Lice (Body lice, Head lice, Pubic lice), Lyme Disease, Malaria, Marburg Hemorrhagic Fever, Measles, Meningitis, Mosquito-borne Diseases, Mycobacterium avium Complex (MAC) Infection, Naegleria Infection, Nosocomial Infections, Nonpathogenic Intestinal Amebae Infection, Onchocerciasis (River Blindness), Opisthorciasis (Opisthorcis Infection), Parvovirus Infection, Plague, PCP (Pneumocystis carinii Pneumonia), Polio, Q Fever, Rabies, Respiratory Syncytial Virus (RSV) Infection, Rheumatic Fever, Rift Valley Fever, Rotavirus Infection, Roundworms Infection, Salmonellosis, Salmonella Enteritidis, Scabies, Shigellosis, Shingles, Sleeping Sickness, Smallpox, Streptococcal Infection, Tapeworm Infection (Taenia Infection), Tetanus, Toxic Shock Syndrome, Tuberculosis, Ulcers (Peptic Ulcer Disease), Valley Fever, Vibrio parahaemolyticus Infection, Vibrio vulnificus Infection, Viral Hemorrhagic Fever, Warts, Waterborne infectious Diseases, West Nile Virus Infection (West Nile Encephalitis), Whooping Cough, and Yellow Fever.

20. The method according to claim 13, wherein the autoimmune disease is selected from the group consisting of:
Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Bald disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS), Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressier's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myelin Oligodendrocyte Glycoprotein Antibody Disorder, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatic, Polymyositis, Postmyocardial infarction syndrome, Postpericardiotomy syndrome, Primary Biliary Cholangitis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Thyroid eye disease (TED), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, and Vogt-Koyanagi-Harada Disease.

21. The method according to claim 20, wherein the autoimmune disease is selected from Lupus nephritis, lupus, systemic lupus erythematosus, myasthenia gravis, multiple sclerosis, polyarthritis, rheumatoid arthritis, irritant sensitivity, psoriasis, asthma, and colitis.

22. The method according to claim 21, wherein the autoimmune disease is myasthenia gravis.

23. A method for producing a compound of the formula (I) comprising:
Step 1A: providing an intermediate compound (I-1*): 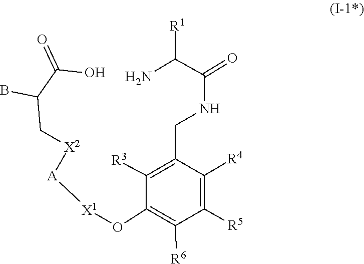
wherein A, B, R¹, R³, R⁴, R⁵, R⁶, X¹, and X² have the same meanings as defined in the formula (I) according to claim 1;
Step 2A: perform an intramolecular amide coupling reaction between a carboxylic acid group and an amine group of the intermediate compound (I-1*) to obtain the compound of the formula (I) 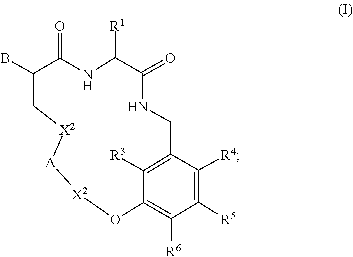
or
a method for producing the compound of the formula (I) comprising:
Step 1B: providing an intermediate compound (I-2*): 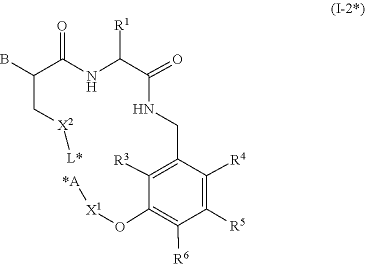
wherein
A* represents —NH(R^{N6})—, 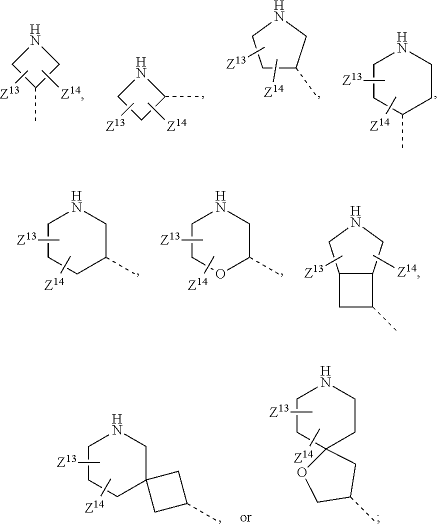
L* represents —CO₂H,
and B, R¹, R³, R⁴, R⁵, R⁶, R^{N6}, X¹, X², Z¹³, and Z¹⁴ have the same meanings as defined in the formula (I) according to claim 1;
Step 2B: perform an intramolecular amide coupling reaction between the L* and an amino group of A* moiety of the intermediate compound (I-2*) to obtain the compound of the formula (I);
or
a method for producing the compound of the formula (I) comprising:
Step 1C: providing an intermediate compound (I-3*): 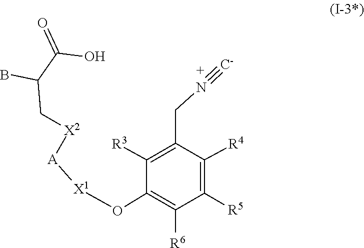
wherein
A, B, R¹, R³, R⁴, R⁵, R⁶, X¹, and X² have the same meanings as defined in the formula (I) according to claim 1;
Step 2C: perform an intramolecular Ugi reaction of the intermediate compound (I-3*) with R¹—CHO and aqueous ammonia (NH₃) to obtain the compound of the formula (I).

24. An intermediate compound selected from the compounds 7*, 8*, 11*, 12*, 13*, 14*,
I-1*, I-2*, and I-3*: 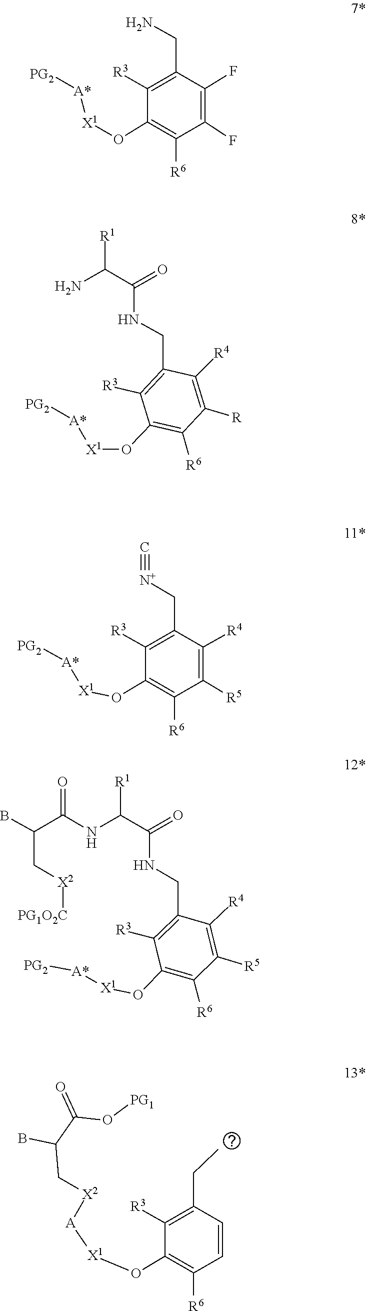 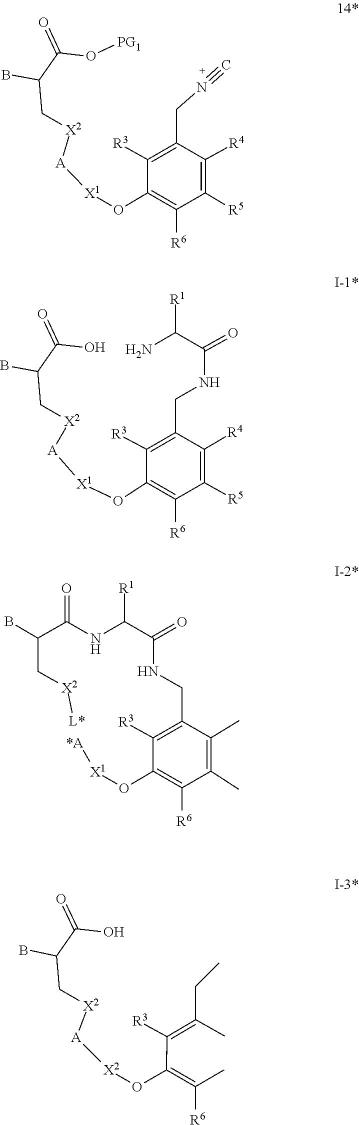
wherein
A* represents —NH(R^{N6})—, 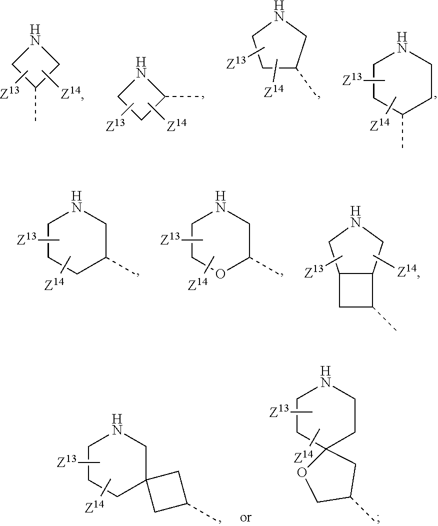
L* represents —CO₂H;
PG₁ represents a carboxyl protecting group;
PG₂ represents an amine protecting group;
and A, B, R¹, R³, R⁴, R⁵, R⁶, R^{N6}, X¹, X², Z¹³, and Z¹⁴ have the same meanings as defined in the formula (I) according to claim 1.
